(19) United States
(12) Patent Application Publication
(10) Pub. No.: US 2010/0168084 A1
(43) Pub. Date: Jul. 1, 2010

(54) Therapeutic compounds and related methods of use

(76) Inventors: L. Julie Huber, Newton, MA (US); Thomas E. McDonagh, Acton, MA (US)

Correspondence Address: LANDO & ANASTASI, LLP, ONE MAIN STREET, SUITE 1100, CAMBRIDGE, MA, 02142, US

(21) Appl. No.: 12/387,960

(22) Filed: May 8, 2009

Related U.S. Application Data
(60) Provisional application No. 61/051,643, filed on May 8, 2008.
(60) Provisional application No. 61/051,645, filed on May 8, 2008.
(60) Provisional application No. 61/051,653, filed on May 8, 2008.
(60) Provisional application No. 61/051,659, filed on May 8, 2008.
(60) Provisional application No. 61/051,656, filed on May 8, 2008.
(60) Provisional application No. 61/051,660, filed on May 8, 2008.
(60) Provisional application No. 61/051,662, filed on May 8, 2008.
(60) Provisional application No. 61/051,666, filed on May 8, 2008.
(60) Provisional application No. 61/051,647, filed on May 8, 2008.
(60) Provisional application No. 61/051,651, filed on May 8, 2008.
(60) Provisional application No. 61/051,667, filed on May 8, 2008.
(60) Provisional application No. 61/051,654, filed on May 8, 2008.
(60) Provisional application No. 61/051,669, filed on May 8, 2008.
(60) Provisional application No. 61/051,650, filed on May 8, 2008.
(60) Provisional application No. 61/051,670, filed on May 8, 2008.
(60) Provisional application No. 61/080,372, filed on Jul. 14, 2008.

(51) Int. Cl. A61K 31/551; A61K 31/195; G01N 33/573; A61K 31/165

(52) U.S. Cl. 514/218; 514/563; 435/7.4; 514/617

(57) ABSTRACT

Methods of using compounds that are HAT inhibitors are described herein.

### CLAIM OF PRIORITY

This application claims priority to U.S. Provisional 61/051,643, filed May 8, 2008; U.S. Provisional 61/051,645, filed May 8, 2008; U.S. Provisional 61/051,653, filed May 8, 2008; U.S. Provisional 61/051,659, filed May 8, 2008; U.S. Provisional 61/051,656, filed May 8, 2008; U.S. Provisional 61/051,660, filed May 8, 2008; U.S. Provisional 61/051,662, filed May 8, 2008; U.S. Provisional 61/051,666, filed May 8, 2008; U.S. Provisional 61/051,647, filed May 8, 2008; U.S. Provisional 61/051,651, filed May 8, 2008; U.S. Provisional 61/051,667, filed May 8, 2008; U.S. Provisional 61/051,654, filed May 8, 2008; U.S. Provisional 61/051,669, filed May 8, 2008; U.S. Provisional 61/051,650, filed May 8, 2008; U.S. Provisional 61/051,670, filed May 8, 2008; U.S. Provisional 61/080,372, filed Jul. 14, 2008; the contents of each of which is incorporated by reference in its entirety.

### BACKGROUND OF INVENTION

Histone acetyl transferases, also known as HATsn are enzymes that acetylate histone proteins by transferring an acetyl group from acetyl CoA. Histone acetylation is generally linked to transcriptional activation. Acetylation and other posttranslational modifications of histones generate binding sites for specific protein-protein interaction domains, such as the acetyl-lysine binding bromodomain. Inhibition of HAT can be useful in the treatment of various diseases or disorders. Examplary HAT inhibitors include curcumin, garcinol, and anacardic acid.

### SUMMARY OF INVENTION

The inventors have discovered that certain compounds are modulators of histone acetyl transferase (HAT) (e.g., inhibitors of HAT). Modulators of HAT (e.g., inhibitors of HAT) can be used to treat various disorders. Exemplary HATs include the following: CBP (CREB-binding protein), p300, pCAF, hGCN5, and ATF2 (activating transcription factor 2).

The inventors have discovered that, surprisingly, compounds described herein are modulators, generally inhibitors, of a HAT. This discovery allows optimization of [symbol] a variety of methods related to these compounds. Of particular importance the discovery allows implementation and/or optimization of methods related to the clinical use of the compounds. E.g., the discovery allows more informed patient selection, instruction, treatment, selection of treatment regimes and monitoring and evaluation of patients. The discovery also allows optimization of making, testing, evaluating, and developing the compounds as well as optimized compositions, preparations and reactions, assay mixtures and reference standards which include these compounds.

The invention allows the selection of patients for treatment with a compound described herein, based on an appreciated need, of the patient, of inhibition of a HAT.

Accordingly, in one aspect, the invention features, a method of selecting or treating a patient for a condition, e.g., cancer, diabetes, or obesity. The method includes:

selecting a patient on the basis of that patient being in need of the inhibition of a HAT for the treatment of the condition; and

selecting, and preferably administering to the subject, a compound of described herein.

The selection step involves selecting the patient on the basis of the appreciation of the patient's need of inhibition of a HAT, as opposed to merely selecting a patient in need of inhibition of a HAT or even selecting a HAT inhibitor based on some basis other than being in need of inhibition of a HAT.

Conditions treatable with methods described herein include chronic and acute conditions and disorders. In embodiments the can be conditions characterized by unwanted HAT activity or unwanted levels of acetylation of a HAT substrate.

The invention also allows optimized evaluation of compounds described herein based on the HAT inhibitory activity of the compounds.

Accordingly, in another aspect, the invention includes a method of evaluating a compound described herein. The method includes:

providing a compound described herein;

providing a value related to the ability of the compound to inhibit a HAT; and

optionally, comparing the value with a reference value, thereby evaluating the compound.

In an embodiment the method further includes selecting the compound, or a batch from which the compound is taken, e.g., for release as an approved drug.

The invention allows optimal characterization and transmittal of information about the characteristics of the compounds described herein.

Accordingly, in another aspect, the invention features a method of identifying characterizing, describing, or providing information on a compound described herein.

The method includes:

identifying, characterizing, describing, or providing information that the drug is a HAT inhibitor, and

identifying the drug as a compound described herein.

In an embodiment of this or any other method herein, the compound is an approved drug.

In another aspect, the invention features, a method of instructing an entity (e.g., a care provider), on the use of a drug of a compound described herein, or of selecting a patient. The method includes:

instructing the entity to select a patient on the basis of that patient being in need of the inhibition of a HAT for the treatment of the condition; and

instructing the entity to provide, e.g., administer a compound described herein, to the patient.

Methods of the invention allow administration of a compound described herein to a patient for treatment of a condition described herein while taking steps to minimize unwanted effects or contraindicated situations.

In another aspect, the invention provides, a method of providing an entity other than the patient, e.g., caregiver, instructions on treating a condition described herein, e.g., cancer, diabetes, or obesity with a compound described herein. The method includes:

instructing the entity to provide or administer a compound described herein; and

instructing the entity to monitor for a side effect that is associated with inhibition of a HAT.

In some embodiments, the side effect is not associated with an HDAC (e.g., a SIRT such as SIRT1).

In another aspect, the invention provides, a method of providing a patient instructions on treating a condition described herein, e.g., cancer, diabetes, or obesity with a compound described herein. The method includes:

instructing the patient to accept or self administer a compound described herein; and

instructing the patient to monitor for a side effect that is associated with inhibition of a HAT, and optionally report any side effect to a caregiver.

In some embodiments, the side effect is not associated with an HDAC (e.g., a SIRT such as SIRT1).

In another aspect, the invention provides, a method of providing an entity other than the patient, e.g., a caregiver, instructions on treating a condition described herein with a compound described herein. The method includes:

instructing the entity to provide or administer a compound described herein; and

instructing the patient or a caregiver to avoid compounds known to interact adversely with an inhibitor of a HAT.

In another aspect, the invention features, a method of treating a patient with a compound described herein for a condition described herein. The method includes:

administering to the patient a compound described herein for the treatment of a condition described herein; and

instructing the patient or a caregiver to avoid compounds known to interact adversely with an inhibitor of a HAT.

A method of treating a patient, the method comprising administering a compound described herein and an activator of an HDAC (e.g., an activator of SIRT, such as SIRT1).

In another aspect, the invention includes a method of approving transfer of payment. The method includes: communicating to the reimburser that the subject is in need of a compound that inhibits a HAT, and instructing or requesting the reimburser to approve or transfer of payment for the treatment of the subject with compound described herein for a disorder described herein.

In another aspect, the invention features, a method of analyzing a preparation of a compound described herein, the method comprising providing the compound described herein and determining whether the compound described herein inhibits HAT, thereby analyzing the drug.

In another aspect, the invention features, a preparation or pharmaceutical preparation of a drug disclosed herein, the preparation comprising at least one carrier, preservative, or excipient, which is suitable for or has been selected for being suitable for an inhibitor of HAT. In an embodiment the preparation excludes CoA co-enzyme, e.g., acetyl-CoA. In an embodiment the preparation excludes a component which would antagonize, interfere with, neutralize, or substantially reduce the effectiveness of a HAT inhibitor but optimally would not antagonize, interfere with, neutralize, or substantially reduce the effectiveness of a sirtuin activator.

In another aspect, the invention features, a reaction mixture for evaluating ability of a composition described herein for inhibiting a HAT. In an embodiment it includes a compound described herein and HAT, or a purified HAT. In an embodiment, the mixture is substantially free of an activator of a sirtuin.

A cell culture comprising a compound described herein and a recombinantly produced HAT.

A method of instructing or facilitating a first party, e.g., a caregiver, a patient, a retailer, or wholesaler, on a use of a drug, the method comprising, instructing the first party that the drug is a HAT inhibitor and instructing the first party that the drug is a compound described herein.

In one aspect, the invention features a method for monitoring the progress of therapeutic treatment with a compound described herein. The method includes determining the level of acetylation of one or more HAT substrates in a biological sample from a subject being greated with a compound described herein, wherein a change in the level of acetylation of the HAT substrate relative to a control is indicative of therapeutic modulation of HAT in the subject. Exemplary HAT substrates include p53 (368-386), which is a cubstrate of CBP and p300 or histone H3 peptide, which is a substrate for pCAF and hGCN5.

In one aspect, the invention features a method for detecting modulation of a HAT in a mammal. The method includes: obtaining a biological sample from a subject that has received a compound described herein, and determining the level of acetylation of one or more HAT substrates in the sample, wherein a change in the level of acetylation of the one or more HAT substrates as compared to a control is indicative of HAT modulation in the mammal. Exemplary HAT substrates include p53 (368-386), which is a cubstrate of CBP and p300 or histone H3 peptide, which is a substrate for pCAF and hGCN5.

In one aspect, the invention features a method of identifying a subject in need of treatment with a HAT inhibiting compound. The method includes determining the level of acetylation of one or more HAT substrates in a biological sample from the subject, wherein a higher level of acetylation in a HAT substrate as compared to a control is indicative of a subject in need of treatment with a HAT inhibiting compound. Exemplary HAT substrates include p53 (368-386), which is a cubstrate of CBP and p300 or histone H3 peptide, which is a substrate for pCAF and hGCN5.

[symbol]

Provided herein are novel modulating compounds and methods of use thereof.

In one aspect, the invention provides [symbol] modulating compounds of Formula (I):

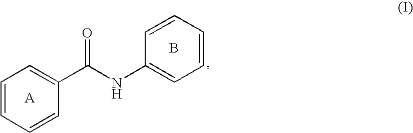

or a salt thereof; where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In another aspect, the invention provides sirtuin-modulating compounds of Formula (II):

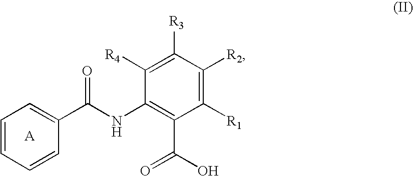

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)R₅, —S(O)ₙOR₅, —S(O)ₙNR₅R₆, —NR₅R₄, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another aspect, the invention provides [symbol] modulating compounds of Formula (III):

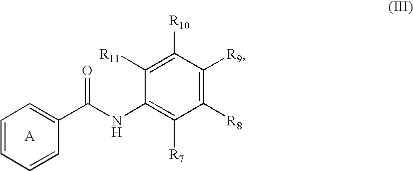

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In another aspect, the invention provides [symbol] modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂; NR₂′C(O)NR₂′R₂; NR₂′C(O)C(O)R₂′; NR₂′C(O)R₂′; NR₂′(COOR₂′); NR₂′C(O)R₅′; NR₂S(O)₂NR₂′R₂′; NR₂′S(O)₂R₂′, NR₂′S(O)₂R₅′; NR₂C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₉′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur, oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₈′; NR₈′R₈′; COOR₇; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀; NR₁₀′OR₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl; each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉, SR₉′, NR₉′R₉, COOR₉′, NO₂, CN, C(O)R₉′, C(O)NR₉′R₉′, NHC(O)R₉′; NH(COOR₉′), S(O)₂NR₉′R₉′, OC(O)R₉′; or R₉′.

In a further aspect, the invention provide [symbol] -modulating compounds of Formula (IVa):

Het-L-Q-Ar′ (IVa)

or a salt thereof, where:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(—NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—CR₁′R₁′—C(O)—NR₁′, —NR₁′—C(O)—CR₁′—CR₁′—CR₁′R₁′—, —NR₁′—C(═N—CN)—NR₁′—, —NR₁′—C(O)—CR₁′R₁′—,

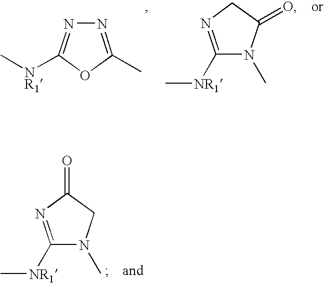

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In still yet another aspect, the invention provides [symbol] modulating compounds of Formula (V):

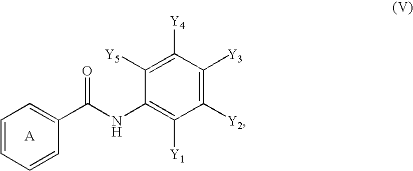

or a salt thereof wherein:
Ring A is optionally substituted with at least one R₁′ group;
Y₁, Y₂, Y₃, Y₄, and Y₅ are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂; OC(O)R₂′; S(O)₂R₂′; S(O)₂NR₂′R₂; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′ C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇, OR₇′, OC(O)R₇′, NR₇′R₇, NR₇′R₅′, NR₈′R₉′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur, oxygen; CF₃; haloalkyl; SR₂′ OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₉′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one aspect, the invention provides [symbol] modulating compounds of Structural Formula (VI):

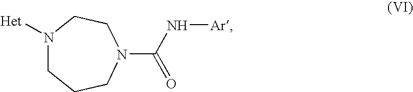

or a salt thereof, wherein:

Het is an optionally substituted heterocyclic aryl group; and

Ar′ is an optionally substituted carbocyclic or heterocyclic aryl group.

The invention also includes prodrugs and metabolites of the compounds disclosed herein.

In another aspect, the invention provides [symbol] -modulating compounds of Structural Formula (VII):

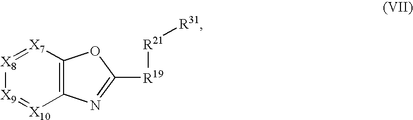

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

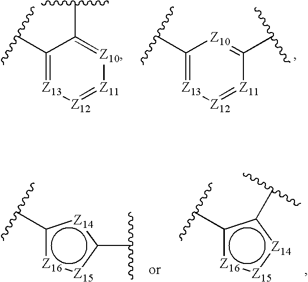

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′═CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

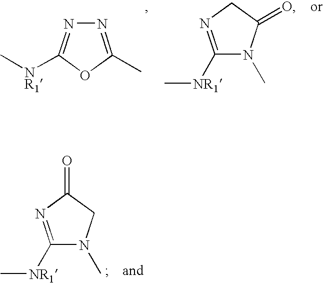

R³¹ is selected from an optionally substituted monocyclic or bicyclic aryl, or an optionally substituted monocyclic or bicyclic heteroaryl, with the provisos that said compound is not:

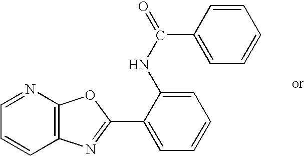

that when R¹⁹ is

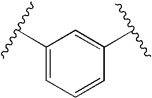

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR², or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

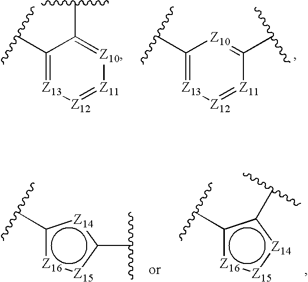

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

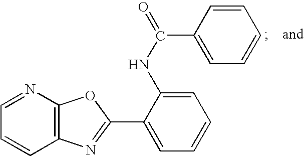

when X₉ and X₉ are each independently selected from CR²⁰ or CR₁′, R¹⁹ is

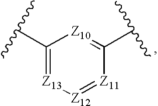

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In yet another embodiment, the invention provides [symbol] modulating compounds of Structural Formula (VIII):

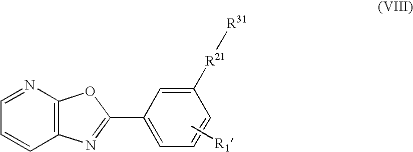

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

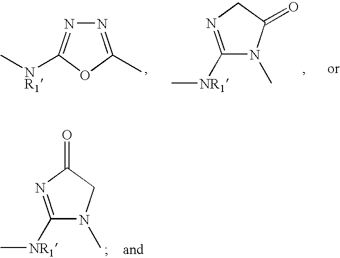

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

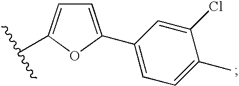

1-methoxynaphthyl; 2-methoxynaphthyl; or unsubstituted 2-thienyl;

when R₁′ is methyl and R²¹ is —NH—C(O)—CH═CH—, R³¹ is not

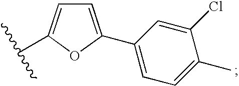

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In a further embodiment, the invention provides [symbol] -modulating compounds of Structural Formula (IX):

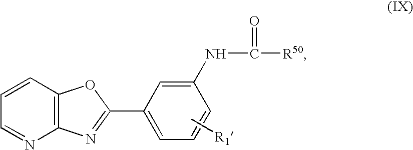

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] modulating compounds of Structural Formula (X):

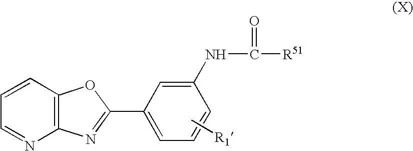

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo[b]thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In another aspect, the invention provide [symbol] modulating compounds of Structural Formula (XI):

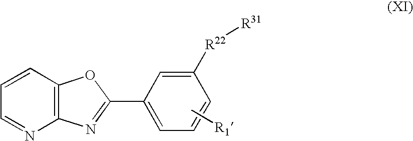

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₇′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In yet another aspect, the invention provides [symbol] modulating compounds of Structural Formula (XII):

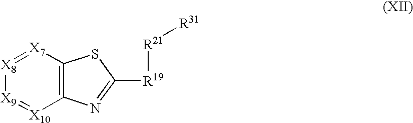

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

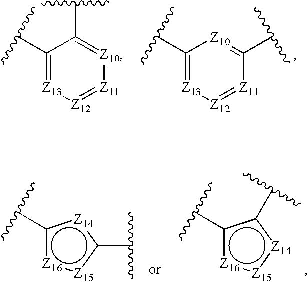

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—, —NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂O—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—R₁′—CR₁′R₁′—, —NR₁′—C(O)—O—,

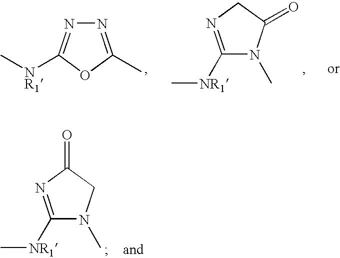

R³¹ is selected from an optionally substituted monocyclic or bicyclic aryl, or an optionally substituted monocyclic or bicyclic heteroaryl, with the proviso that when R¹⁹ is

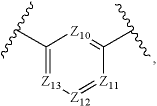

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

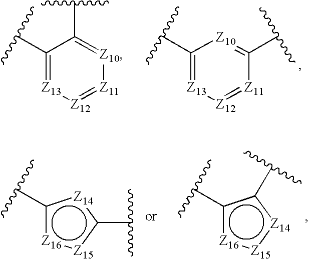

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

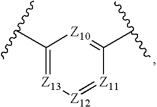

and each of Z₁₀, Z₁₁, Z₁₂ and Z₁₃ is independently selected from CR²⁰, or CR₁′, then:
a) at least one of X, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In a further aspect, the invention provides compounds of Structural Formula (XIII).

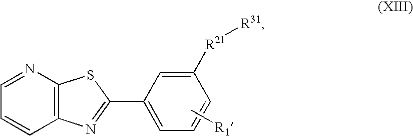

or a s thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—; —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

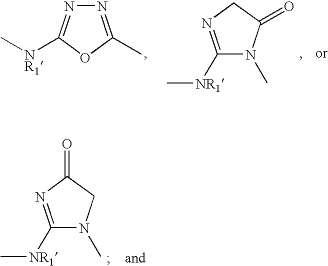

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one aspect, the invention provides [symbol] -modulating compounds of Structural Formula (XIV):

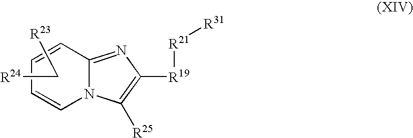

or a salt thereof; wherein:

each of R²³ and R²⁴ is independently selected from H, —CH₃ or a solubilizing group;

R²⁵ is selected from H, or a solubilizing group; and

R¹⁹ is selected from:

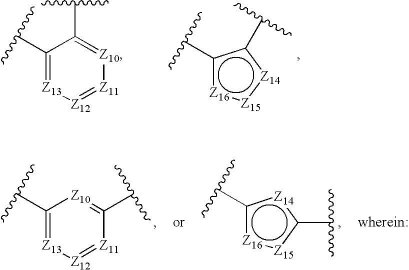

each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′, —CR₁′R₁′—, —NR₁′—C(S)—NR₁′—CR₁′R₁′—CR₁′R₁′—, —NR₁′—C(O)—O—,

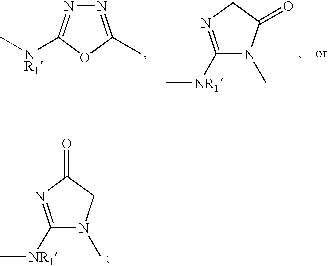

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic hetero r
wherein when R¹⁹ is

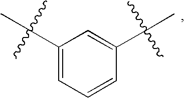

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides [symbol] modulating compounds of Structural Formula (XV):

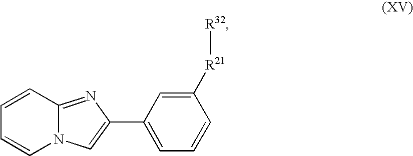

or a salt thereof; wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(═N—CN)—NR₁′, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₃′—CR₁′R₁′—, —NR₁′—C(O)—CR₁′R₁′—; —NR₁′—C(O)—CR₁′R₁′—CR₁′R₁′—, —NR₁′R₁′—C(S)—NR₁′—CR₁′R₁′—CR₁′R₁′—CR₁′R₁′—, —NR₁′—C(O)—O—,

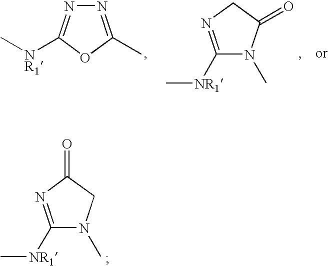

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

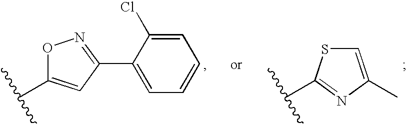

and

when R²¹ is —NR₁′—S(O)₂—, R³² is not unsubstituted 2-thienyl or unsubstituted naphthyl.

In yet another aspect, the invention provides [symbol] modulating compounds of Structural Formula (XVI):

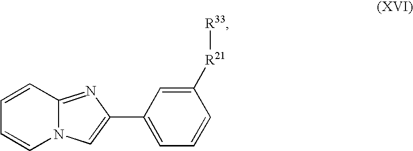

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

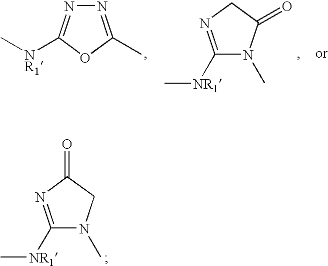

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further aspect, the invention provides [symbol] -modulating compounds of Structural Formula (XVII):

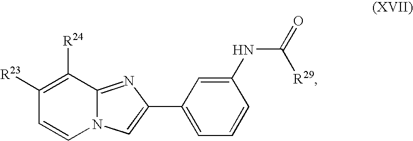

or a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position, wherein R²⁹ is optionally additionally substituted with a solubilizing group.

In one aspect, the invention provides [symbol] modulating compounds of Structural Formula (XVIII):

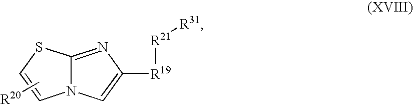

or a salt thereof, wherein

R¹⁹ is selected from:

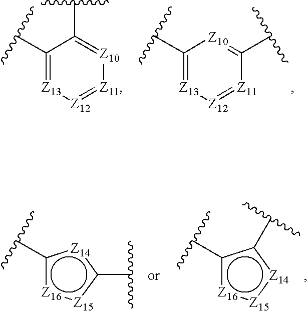

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₂′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁₁—, —NR₁′—C(S)—NR₁′—CR₁′R₁′—CR₁′R₁′—, —NR₁′—C(O)—O—,

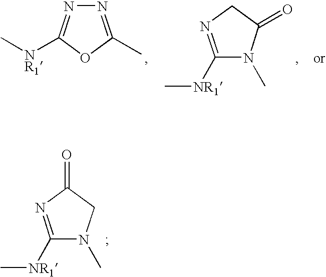

wherein each R₁′ is independently selected from H optionally substituted C₁-C₃ straight or branched alkyl; and
R³¹ is selected from an optionally substituted monocyclic or bicyclic aryl, or an optionally substituted monocyclic or bicyclic heteroaryl, with the proviso that when R¹⁹ is

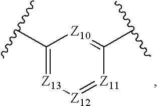

Z₁₀, Z₁₁, Z₁₁, Z₁₂ and Z₁₃ are each CH, R²⁰ is H, and R²¹ is —NHC(O)—, R³¹ is not an optionally substituted phenyl.

In another aspect, the invention provides [symbol] -modulating compounds of Structural Formula (XX):

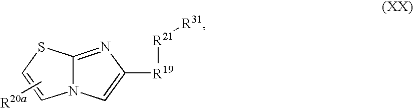

or a salt thereof, rein

R¹⁹ is selected from:

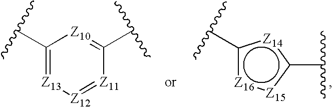

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂O—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁₁—CR₁′R₁₁—, —NR₁′—C(O)—O—,

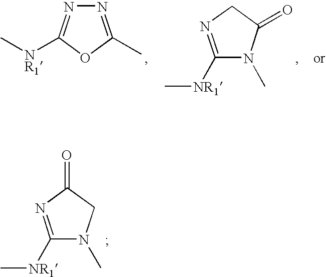

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

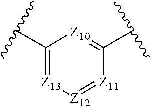

and Z₁₀, Z₁₁, Z₁₂ and Z₁₃ are each CH, R^{20a} is a solubilizing group.

In yet another aspect, the invention provides [symbol] modulating compounds of Structural Formula (XXI):

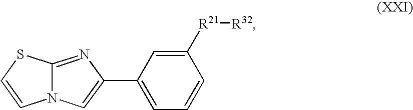

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₂′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—

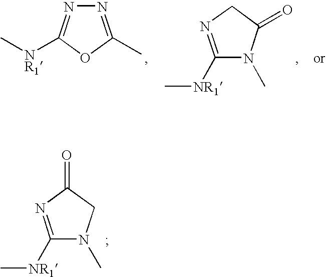

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In a further aspect, the invention provides [symbol] modulating compounds of Structural Formula (XXII):

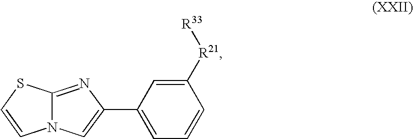

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—R₁′—CR₁′R₁′—CR₁′R₁′, —NR₁′—C(O)—O—,

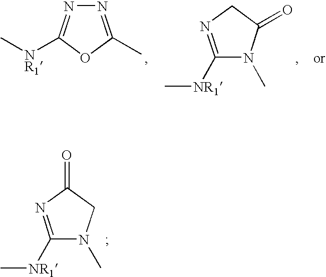

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one aspect, the invention provides [symbol] -modulating compounds of Structural Formula (XXII):

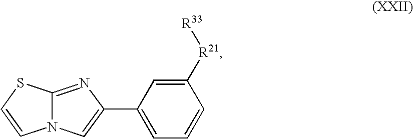

or a salt thereof wherein:

R²¹ is selected from —NH—C(O)—, or —NH—C(O)—CH₂—; and

R³³ is phenyl substituted with

a) one —N(CH₃)₂ group;

b) one CN group at the 3 position;

c) one —S(CH₃) group; or

d)

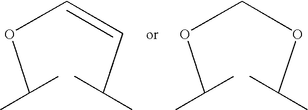

bridging the 3 and 4 positions.

In another aspect, the invention provides [symbol] modulating compounds of Structural Formula (XXIII):

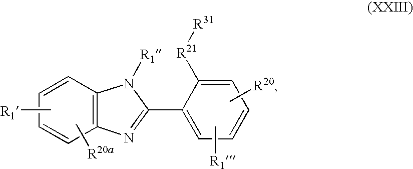

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)—, —NR₁′—CR₁′R₁′—C(O)—NR₁′—, —NR₁′—C(O)—CR₁′═CR₁′═CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl

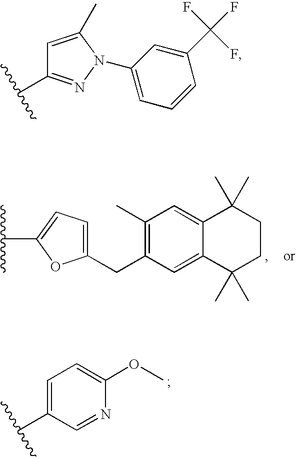

when R²¹ is —NH—C(O)— and each of R²⁰, R^{20a}, R₁′, R₁″ and R₁′″ is hydrogen, R³¹ is not

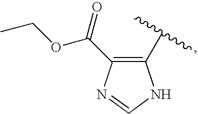

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁′″ is hydrogen, R³¹ is not 2-methylaminophenyl,

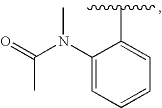

or

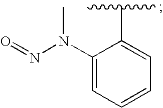

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R¹′, R₁″ and R₁′″ is hydrogen, R³¹ is not

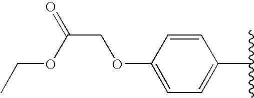

or

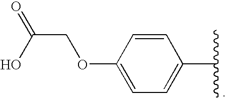

In a particular aspect, the invention provides [symbol] modulating compounds of Structural Formula (XXIII):

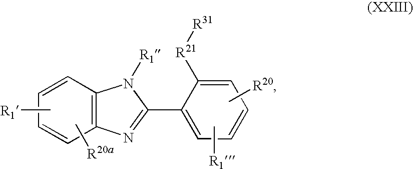

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—NR₁′—C(O)—CR₁′R₁′—NR₁′—, —NR₁′—C(═NR₁′)—NR₁′—, —NR₁′—C(O)—CR₁′═CR₁′—, —NR₁′—S(O)_{z}—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In yet another aspect, the invention provides [symbol] modulating compounds of Structural Formula (XXIV):

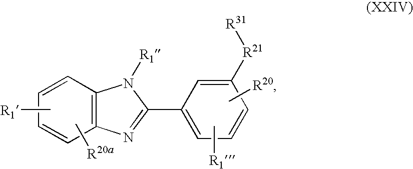

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′═CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

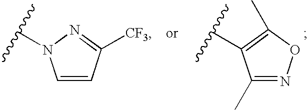

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] -modulating compounds of Structural Formula (XXIV):

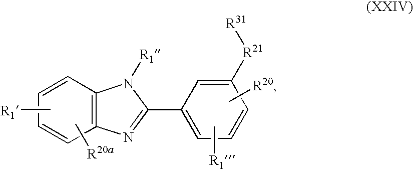

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²¹—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In a further aspect, the invention [symbol] sirtuin-modulating compounds of Structural Formula (XXV):

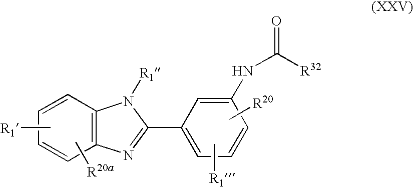

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one aspect, the invention provides [symbol] -modulating compounds of Structural Formula (XXVI):

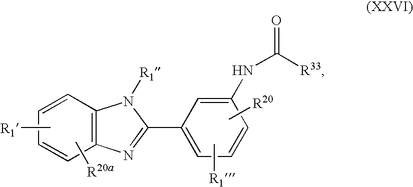

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

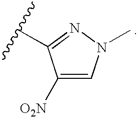

In a particular aspect, the invention provides [symbol] modulating compounds of Structural Formula (XXVI):

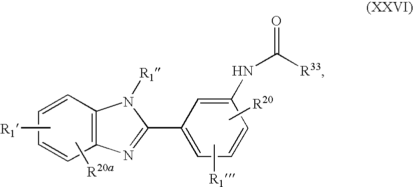

or a salt thereof, wherein:

each R²⁰ and R^{20a} is independently selected from H or a solubilizing group wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] modulating compounds of Structural Formula (XXVII):

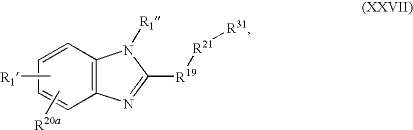

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

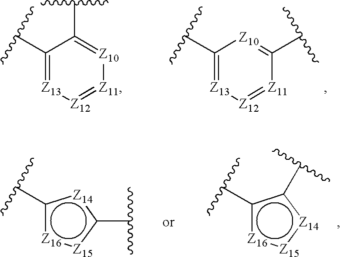

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R₃₁ is selected from an optionally substituted monocyclic or bicyclic aryl, or an optionally substituted monocyclic or bicyclic heteroaryl,
provided that when R²¹ is —NH—C(O)— and R¹⁹ is

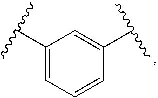

R³¹ is not unsubstituted pyridyl, 2,6-dimethoxyphenyl, 3,4,5-trimethoxyphenyl or unsubstituted furyl.

In a particular aspect, the invention provides [symbol] modulating compounds of Structural Formula (XXVII):

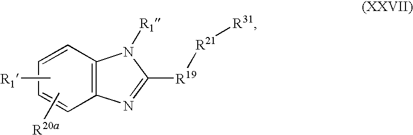

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is s

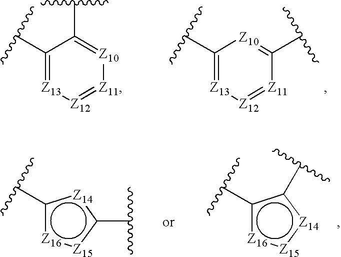

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁ R₁′—, —NR₁′—S(O)₂—CR₁′R₁′—CR₁′R₁′—, or —NR₁′—C(O)—CR₁′R₁′—; and

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

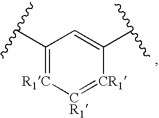

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

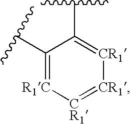

R³¹ is not 3,5-dinitrophenyl, 4-butoxyphenyl,

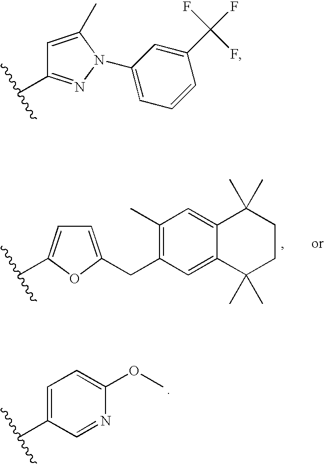

In a more particular embodiment, the invention provides [symbol] -modulating compounds of Structural Formula (XXVII):

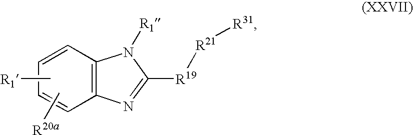

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

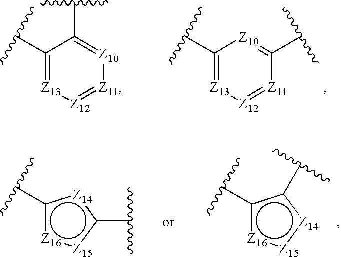

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazolyl or pyrimidinyl, R³¹ is not unsubstituted phenyl.

In yet another aspect, the invention provides compounds of Structural Formula (XXVIII):

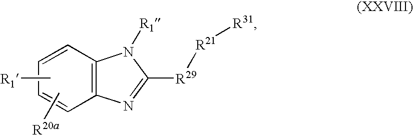

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

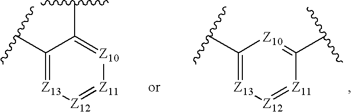

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In one aspect, the invention provides [symbol] -modulating compounds of Formula (I):

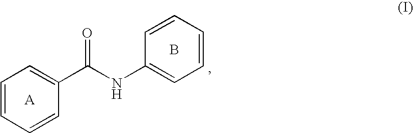

or a salt thereof, where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In another aspect, the invention provides [symbol] -modulating compounds of Formula (II):

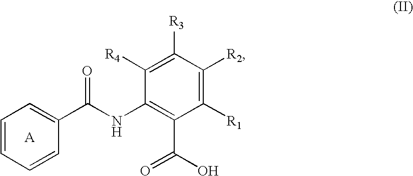

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)OR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another aspect, the invention provides [symbol] -modulating compounds of Formula (III):

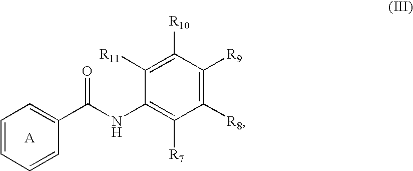

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In another aspect, the invention provides [symbol] -modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl-substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₄′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₉′; NR₉′C(O)C(O)NR₉′R₉′; C1-C10 alkyl substituted with 1-3 independent R₆′, halo, CF₃, OR₉′, SR₉′, NR₉′R₁′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a further aspect, the invention provides [symbol] -modulating compounds of Formula (IVa):

Het-L-Q-Ar′ (IVa)

or a salt thereof, where:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁₁—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—,

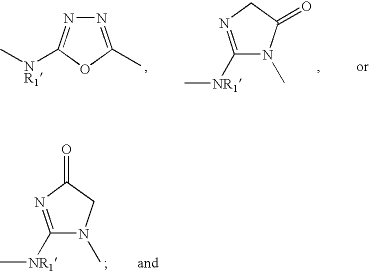

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In still yet another aspect, the invention provides [symbol] -modulating compounds of Formula (V):

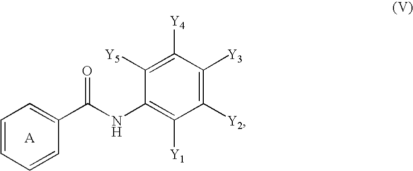

or a salt thereof, wherein:

Ring A is optionally substituted with at least one R₁′ group;

Y₁, Y₂, Y₃, Y₄, and Y₅ are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂); NR₂′C(O)R₅′; NR₂′S(O)₂NR₂′R₂′; NR₂′S(O)₂R₂; NR₂′S(O)₂R₅′; NR₂′C(O)C(O)NR₂′R₂′; NR₂′C(O)C(O)NR₂′ R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂₁; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀ , or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉; C1-C 0 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉, OC(O)R₉′; C2-C10 alkenyl substituted with 1-3 independent R₆′, halo, CF₃, OR₉′, SR₉′, NR₉R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one aspect, the invention provides [symbol] modulating compounds of Structural Formula (VI):

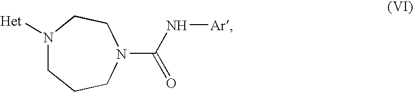

or a salt thereof, wherein:

Het is an optionally substituted heterocyclic aryl group; and

Ar′ is an optionally substituted carbocyclic or heterocyclic aryl group.

The invention also includes prodrugs and metabolites of the compounds disclosed herein.

In another aspect, the invention provides [symbol] -modulating compounds of Structural Formula (VII):

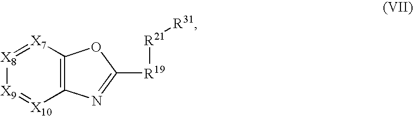

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

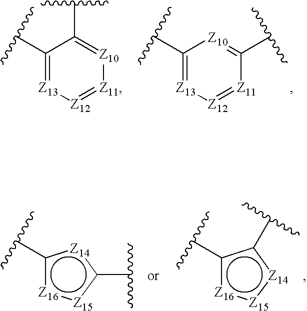

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C1-C3 straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)—CR₁′R₁′—CR₁′R₁′—, —NR₁′—C(O)—CR₁′R₁′—; —NR₁′—C(O)—CR₁′R₁′—CR₁′R₁₁—, —NR₁′—C(S)—NR₁′—CR₁′R₁′—CR₁′R₁′—, —NR₁′—C(O)—O—,

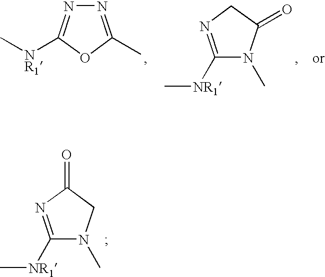

and

R³¹ is selected from an optionally substituted monocyclic or bicyclic aryl, or an optionally substituted monocyclic or bicyclic heteroaryl, with the provisos that said compound is not

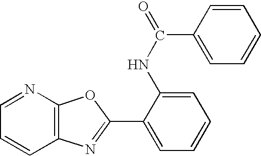

or
that when R¹⁹ is

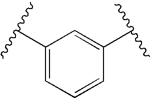

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

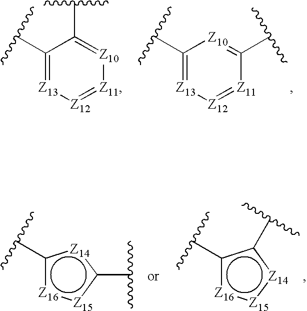

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

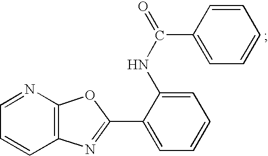

and

when Xs and X₉ are each independently selected from CR²⁰ or CR₁′, R¹⁹ is

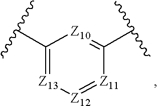

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In yet another embodiment, the invention provides [symbol] -modulating compounds of Structural Formula (VIII):

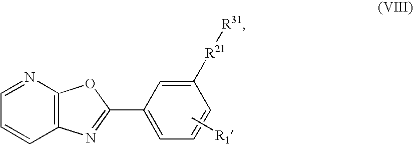

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

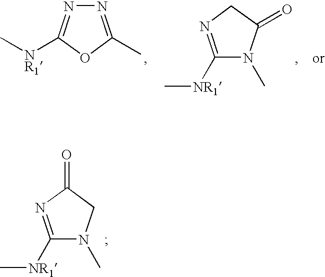

and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

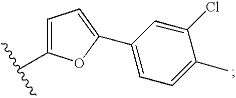

1-methoxynaphthyl; 2-methoxynaphthyl; or unsubstituted 2-thienyl;

when R₁′ is methyl, and R²¹ is —NH—C(O)—CH═CH—, R³¹ is not

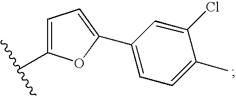

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In a further embodiment, the invention provides [symbol] -modulating compounds of Structural Formula (IX):

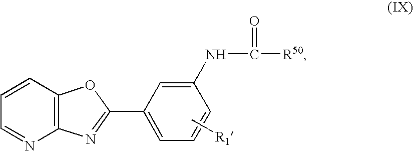

or a salt thereof; wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] modulating compounds of Structural Formula (X):

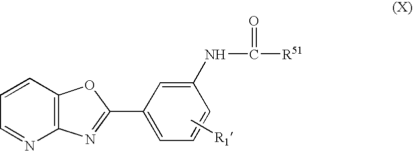

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo[b]thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl; unsubstituted naphthyl, methoxy-, methyl-, or halo-naphthyl, unsubstituted thienyl, unsubstituted pyridinyl, or chloropyridinyl.

In another aspect, the invention provides [symbol] modulating compounds of Structural Formula (XI):

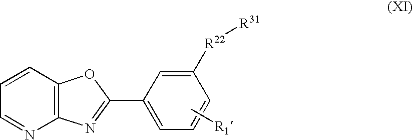

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—C(S)—NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In yet another aspect, the invention provides [symbol] -modulating compounds of Structural Formula (XII):

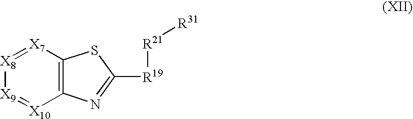

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

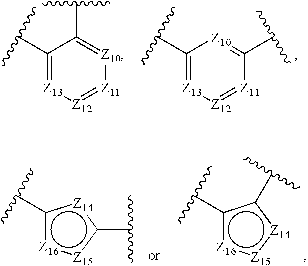

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁—; —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

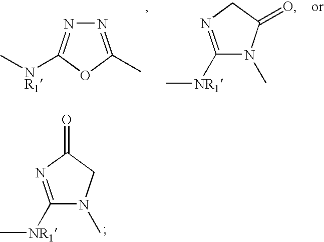

and

R³¹ is selected from an optionally substituted monocyclic or bicyclic aryl, or an optionally substituted monocyclic or bicyclic heteroaryl, with the proviso that when R¹⁹ is

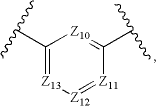

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

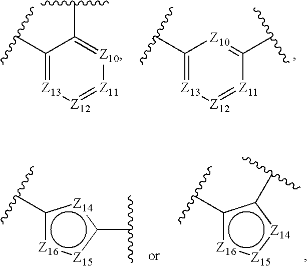

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

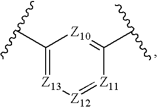

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In a further aspect, the invention provides compounds of Structural Formula (XIII):

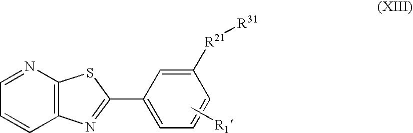

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

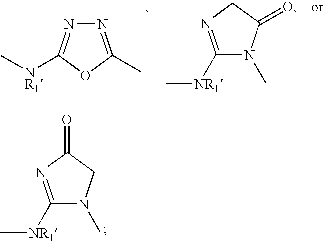

and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-bimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one aspect, the invention provides [symbol] modulating compounds of Structural Formula (XIV):

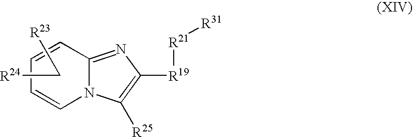

or a salt thereof, wherein:

each of R²³ and R²⁴ is independently selected from H, —CH₃ or a solubilizing group;

R²⁵ is selected from H, or a solubilizing group; and

R¹⁹ is selected from:

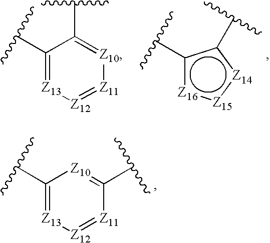

or

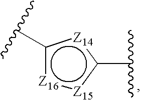

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR¹′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

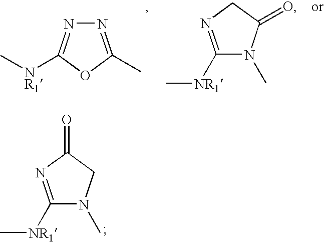

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

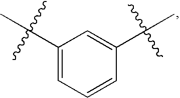

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides [symbol] modulating compounds of Structural Formula (XV):

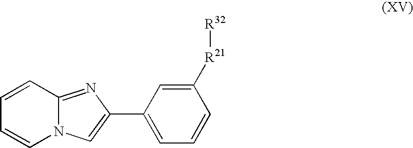

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

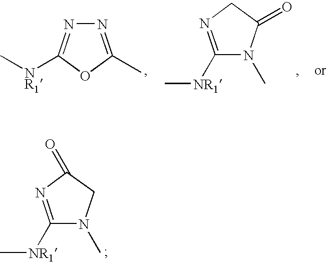

and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

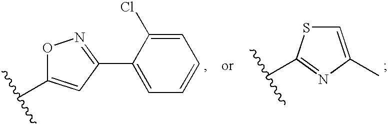

and

when R²¹ is —NR₁′—S(O)₂—, R₃₂ is not unsubstituted 2-thienyl or unsubstituted naphthyl.

In yet another aspect, the invention provides [symbol] modulating compounds of Structural Formula (XVI):

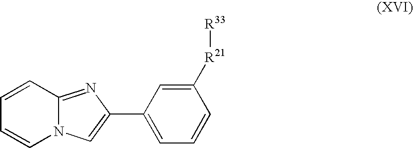

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

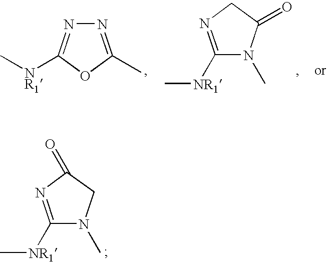

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further aspect, the invention provides [symbol] modulating compounds of Structural Formula (XVII):

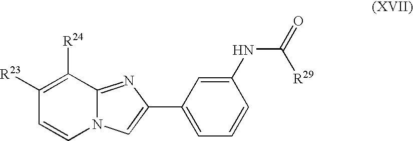

or a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position, wherein R²⁹ is optionally additionally substituted with a solubilizing group.

In one aspect, the invention provides [symbol] modulating compounds of Structural Formula (XVIII):

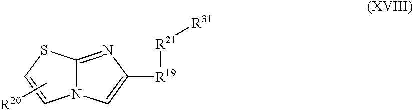

or a salt thereof, wherein

R¹⁹ is selected from:

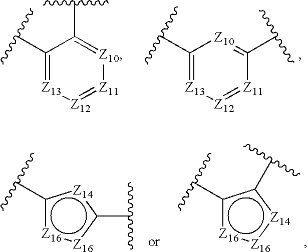

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

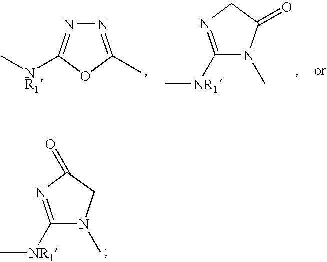

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

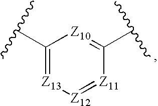

Z₁₀, Z₁₁, Z₁₂ and Z₁₃ are each CH, R²⁰ is H, and R²¹ is —NHC(O)—, R³¹ is not an optionally substituted phenyl.

In another aspect, the invention provides [symbol] -modulating compounds of Structural Formula (XX):

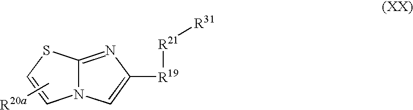

or a salt thereof, wherein

R¹⁹ is selected from:

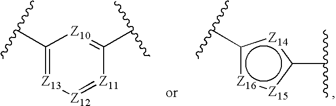

wherein:
each Z₁₀, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁₁—, —NR₁′—C(S)—NR₁′—CR₁′R₁′—CR₁′R₁′—, —NR₁′—C(O)—O—,

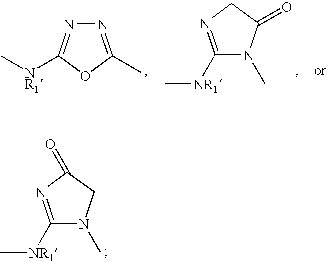

wherein
each R₁′ is independently selected from H or optionally substituted C₁-C₃ straight or branched alkyl; and

R¹³ is selected from an optionally substituted monocyclic or bicyclic aryl, or an optionally substituted monocyclic or bicyclic heteroaryl, wherein when R¹⁹ is

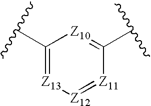

and Z₁₀, Z₁₁, Z₁₂ and Z₁₃ are each CH, R^{20a} is a solubilizing group.

In yet another aspect, the invention provides [symbol] modulating compounds of Structural Formula (XXI):

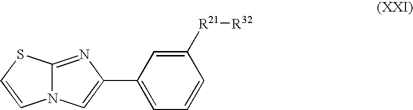

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

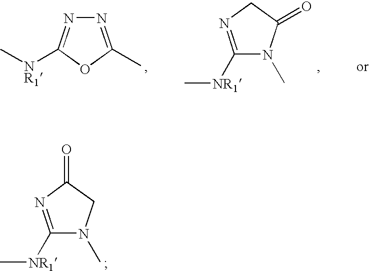

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In a further aspect, the invention provides [symbol] -modulating compounds of Structural Formula (XXII):

or a salt thereof, wherein:

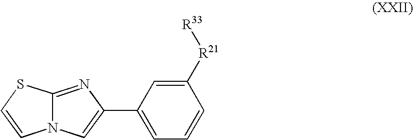

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

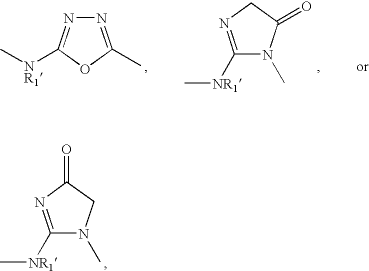

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one aspect, the invention provides [symbol] modulating compounds of Structural Formula (XXII):

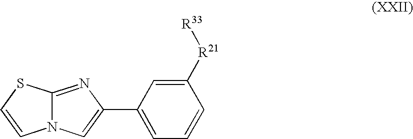

or a salt thereof wherein:

R²¹ is selected from —NH—C(O)—, or —NH—C(O)—CH₂—; and

R³³ is phenyl substituted with

a) one —N(CH₃)₂ group;

b) one CN group at the 3 position;

c) one —S(CH₃) group; or

d)

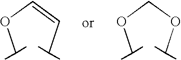

bridging the 3 and 4 positions.

In another aspect, the invention provides [symbol] modulating compounds of Structural Formula (XXIII):

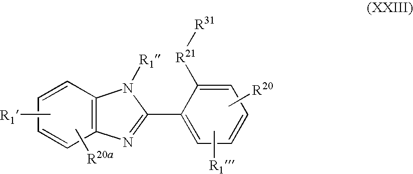

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

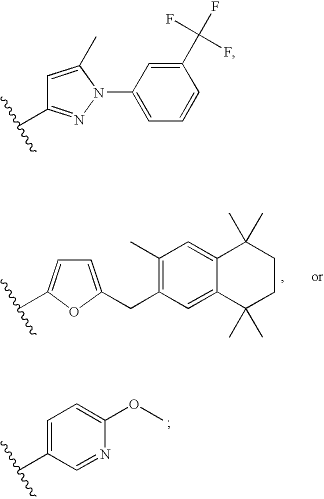

or

when R² is —NH—C(O)— and each of R²⁰, R^{20a}, R₁′, R₁″ and R₁′″ is hydrogen, R³¹ is not

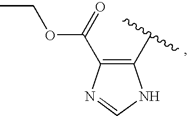

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁′″ is hydrogen, R³¹ is not 2-methylaminophenyl,

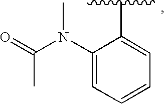

or

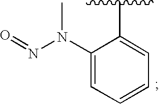

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

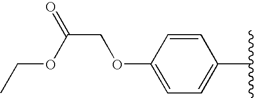

or

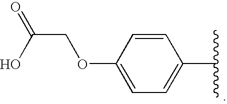

In a particular aspect, the invention provides [symbol] modulating compounds of Structural Formula (XXIII):

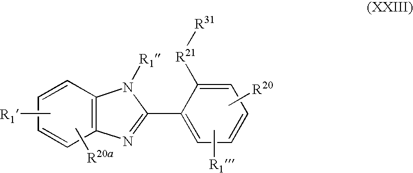

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In yet another aspect, the invention provides [symbol] -modulating compounds of Structural Formula (XXIV):

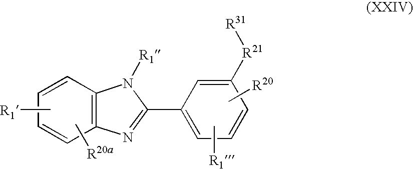

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(—NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

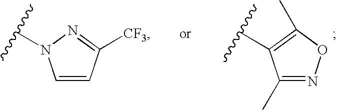

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] modulating compounds of Structural Formula (XXIV):

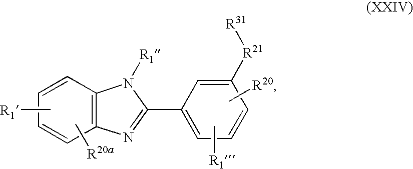

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In a further aspect, the invention provides [symbol] modulating compounds of Structural Formula (XXV):

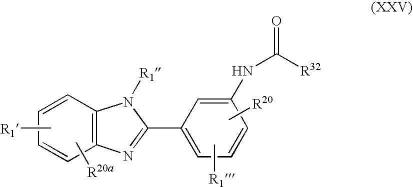

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one aspect, the invention provides [symbol] modulating compounds of Structural Formula (XXVI):

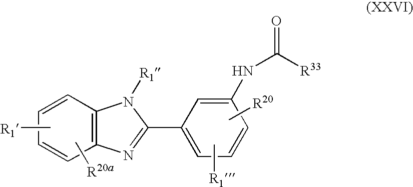

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

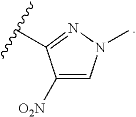

In a particular aspect, the invention provides [symbol] -modulating compounds of Structural Formula (XXVI):

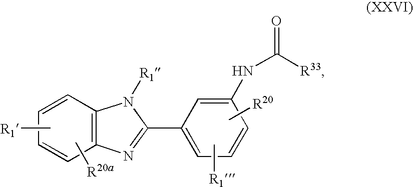

or a salt thereof, wherein:

each R²⁰ and R^{20a} is independently selected from H or a solubilizing group, wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] modulating compounds of Structural Formula (XXVII):

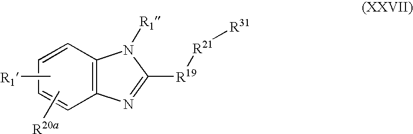

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

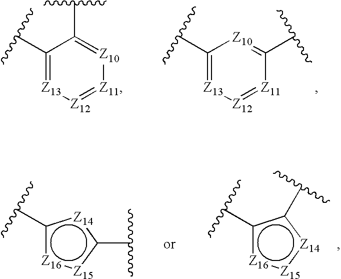

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

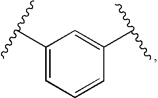

R³¹ is not unsubstituted pyridyl, 2,6-dimethoxyphenyl, 3,4,5-trimethoxyphenyl or unsubstituted furyl.

In a particular aspect, the invention provides [symbol] -modulating compounds of Structural Formula (XXVII):

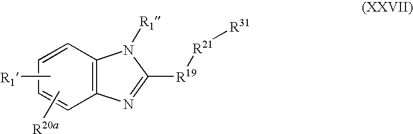

or a salt thereof; wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

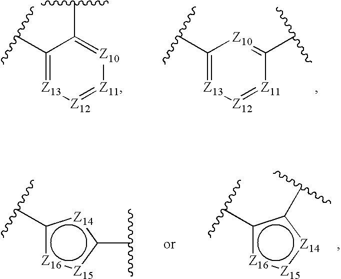

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

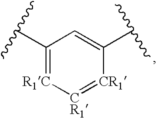

R³¹ is not 2-methylphenyl or 3,4-dimethoxyphenyl;

when R²¹ is —NH—C(O)—CH═CH—, and R¹⁹ is

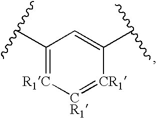

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

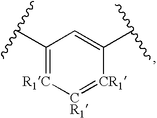

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

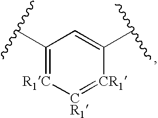

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

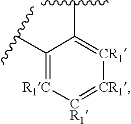

R³¹ is not 3,5-dinitrophenyl, 4-butoxyphenyl,

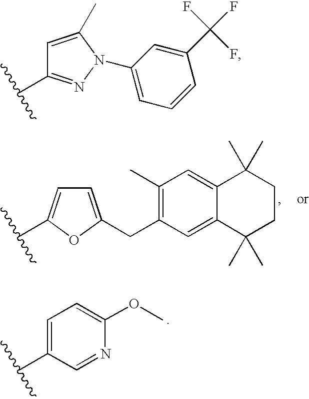

In a more particular embodiment, the invention provides [symbol] modulating compounds of Structural Formula (XXVII):

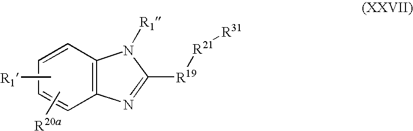

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

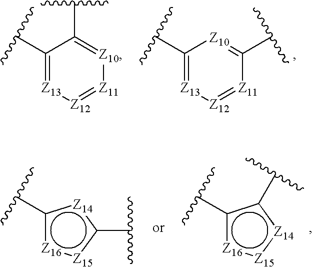

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl; R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ⁱ methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazoyl or pyrimidinyl, R³¹ is not unsubstituted phenyl.

In yet another aspect, the invention provides compounds of Structural Formula (XXVIII):

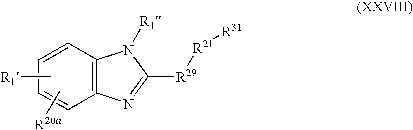

or a salt thereof, wherein:

each R²⁰ and R^{20a} is independently selected from H or a solubilizing group;

each R₁′ and R₁″ independently selected from H or optionally substituted C₁-C₃ straight or branched, alkyl;

R²⁹ is selected from:

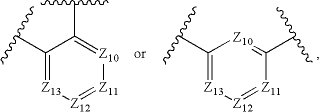

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR¹′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In one aspect, the invention provides [symbol] modulating compounds of Formula (I):

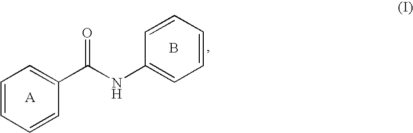

or a salt thereof, where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In another aspect, the invention provides [symbol] -modulating compounds of Formula (II):

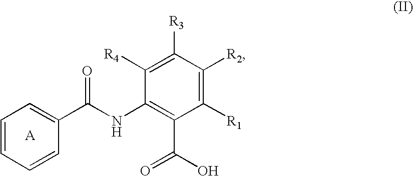

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another aspect, the invention provides [symbol] modulating compounds of Formula (II):

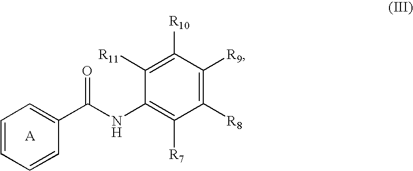

or a salt thereof where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₅, —NR₅R₆, —NR₅C(O)OR₆, —NR₅C(O)R₆ and —NO₂;

R₈ is a polycyclic aryl group; and

n is or 2.

In another aspect, the invention provides [symbol] -modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′ R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl; each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a further aspect, the invention provides [symbol] -modulating compounds of Formula (IVa):

Het-L-Q-Ar′ (IVa)

or a salt thereof; where:
Het is an optionally substituted heterocyclic aryl group;
L is an optionally substituted carbocyclic or heterocyclic arylene group;
Ar′ is an optionally substituted carbocyclic or heterocyclic aryl group; and
Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—,

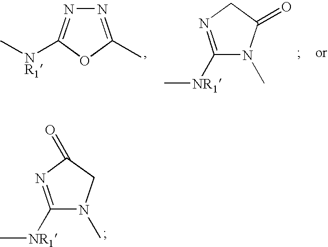

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In still yet another aspect, the invention provides [symbol] -modulating compounds of Formula (V):

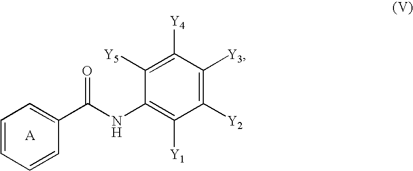

or a salt thereof, wherein:

Ring A is optionally substituted with at least one R₁′ group;

Y₁, Y₂, Y₃, Y₄, and Y₅ are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂); NR₂′C(O)R₅′; NR₂S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₁′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′ or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′, OR₇′; NR₇′R₇′; NR₇′R₇′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₉′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′, S(O)₂NR₉′R₉′, OC(O)R₉′; C2-C10 alkenyl substituted with 1-3 independent R₆, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one aspect, the invention provides [symbol] -modulating compounds of Structural Formula (VI):

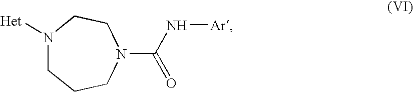

or a salt thereof, wherein.

Het is an optionally substituted heterocyclic aryl group; and

Ar′ is an optionally substituted carbocyclic or heterocyclic aryl group.

The invention also includes prodrugs and metabolites of the compounds disclosed herein.

In another aspect, the invention provides [symbol] modulating compounds of Structural Formula (VII):

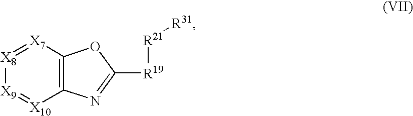

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

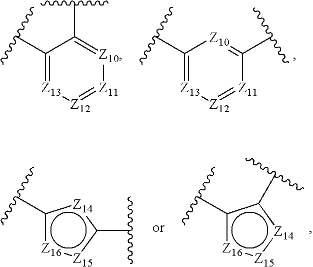

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R₁′ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′R₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′, —CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

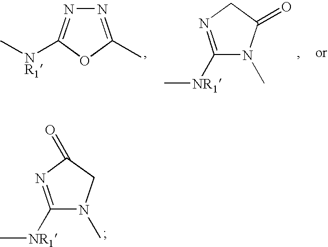

and

R³¹ is selected from an optionally substituted monocyclic or bicyclic aryl, or an optionally substituted monocyclic or bicyclic heteroaryl, with the provisos that said compound is not:

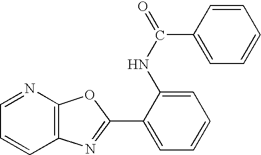

or that when R¹⁹ is

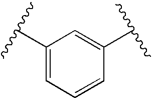

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

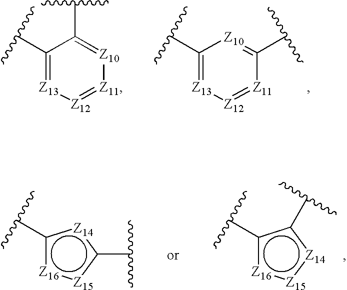

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ s selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁—, —NR₁′—S(O)₂—NR₁′—, —NR₁′—C(O)—NR₁′—S(O)₂—, —NR₁′—CR₁′R₁′—C(O)—NR₁′—, —CR₁′R₁′, —C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

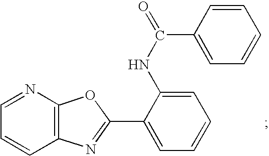

and

when X₈ and X₉ are each independently selected from CR²⁰ or CR₁′, R¹⁹ is

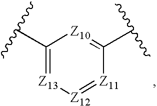

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In yet another embodiment, the invention provides [symbol] modulating compounds of Structural Formula (VIII):

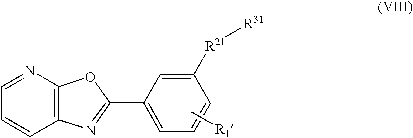

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′R₁′—, —NR₁′—S(O)₂—NR₁′—, —NR₁′—C(O)—NR₁′—S(O)_{z}—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

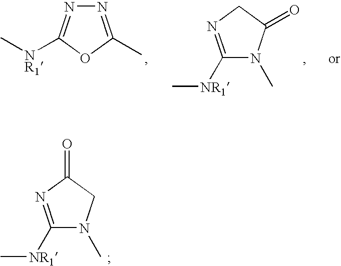

and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

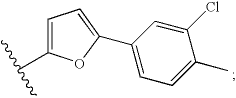

1-methoxynaphthyl; 2-methoxynaphthyl; or unsubstituted 2-thienyl;

when R₁′ is methyl, and R²¹ is —NH—C(O)—CH═CH—, R³¹ is not

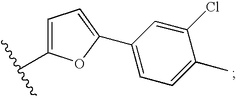

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In a further embodiment, the invention provides [symbol] modulating compounds of Structural Formula (IX):

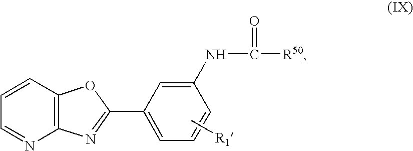

or a salt thereof wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

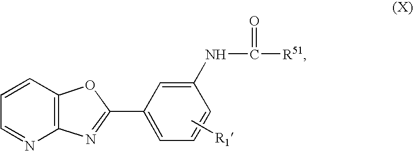

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo[b]thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In another aspect, the invention provides [symbol] modulating compounds of Structural Formula (XI):

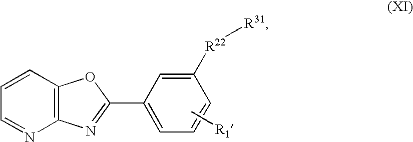

or a salt thereof, wherein:

R₁′ is selected from H or optionally substituted C₁-C₃ straight or branched alkyl;

R²¹ is selected from —NR²³—C(O—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—N₁′R₁′—S(O)₂—, —NR₁—CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 44 chlorophenyl, or unsubstituted phenyl; and

when R²² is —NH—S(O)₂—, R³¹ is not unsubstituted phenyl.

In yet another aspect, the invention provides [symbol] -modulating compounds of Structural Formula (XII):

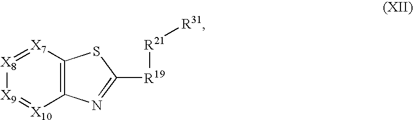

or a salt thereof, wherein:
each of X₇, X₈, X₉ and X₁₀ is independently selected from N, CR², or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR² or CR₁′; and
zero to one R²⁰ is a solubilizing group;

R¹⁹ is selected from:

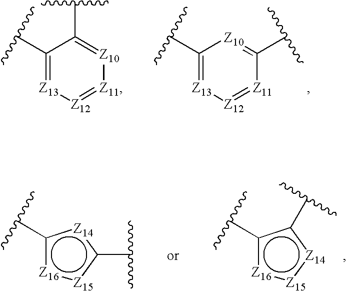

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁—C(S)—NR₁′—CR₁′R₁′—CR₁′R₁′, —NR₁′—C(O)—O—,

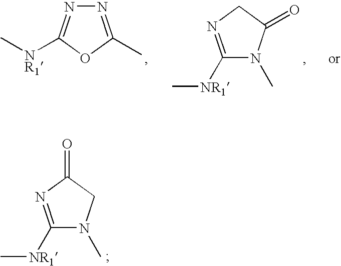

and

R³¹ is selected from an optionally substituted monocyclic or bicyclic aryl, or an optionally substituted monocyclic or bicyclic heteroaryl, with the proviso that when R¹⁹ is

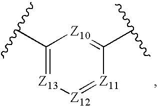

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

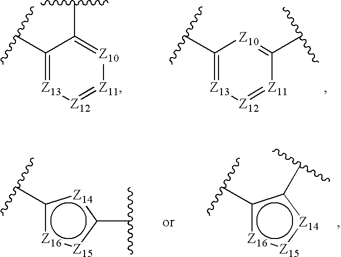

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁—, —NR₁′—C(O)—CR₁′R₁′, —CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

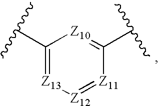

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In a further aspect, the invention provides compounds of Structural Formula (XIII):

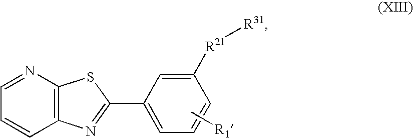

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

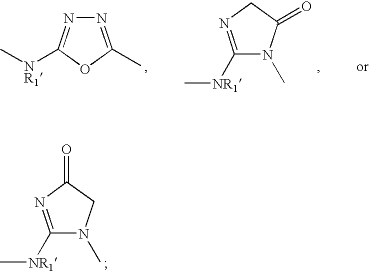

and

R³¹ is selected from an optionally substituted monocyclic or bicyclic aryl, or an optionally substituted monocyclic or bicyclic heteroaryl, with the provisos that

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one aspect, the invention provides [symbol] modulating compounds of Structural Formula (XIV):

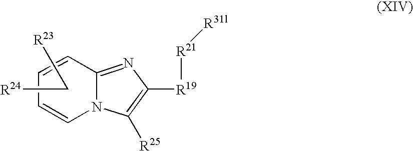

or a salt thereof wherein:

each of R²² and R²⁴ is independently selected from H, —CH₃ or a solubilizing group;

R²⁵ is selected from H, or a solubilizing group; and

R¹⁹ is selected from:

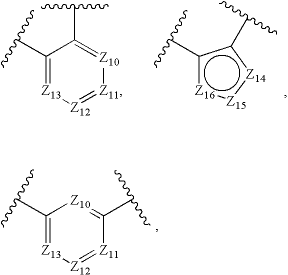

or

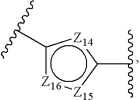

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)—, —NR₁′—C(O)—NR₁′—, —NR₁′—C(S)—NR₁′—, —NR₁′—C(S)—NR₁′—CR₁′R₁′—, —NR₁′—C(O)—CR¹′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—NR₁′—S(O)₂—NR₁′—, —NR₁′—C(O)—NR₁′—S(O)—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—, —CR₁′R₁′—, —NR₁′—C(S)—NR₁—CR₁′R₁′—CR₁′R₁′—, —NR₁′—C(O)—O—,

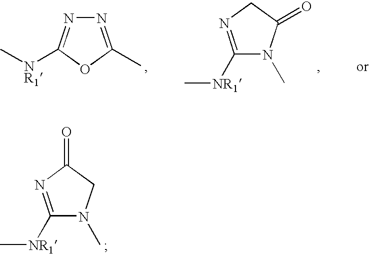

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

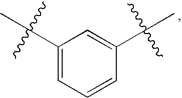

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides [symbol] modulating compounds of Structural Formula (XV):

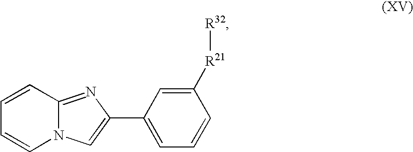

or a salt thereof wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

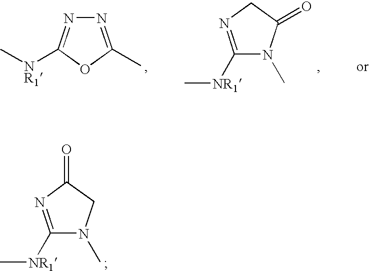

and

each R₁′ is independently selected from H or optionally substituted C₁-C₃ straight or branched alkyl; and

R³² is selected from an Optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

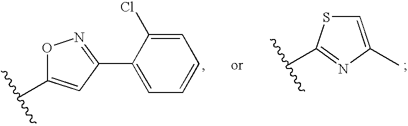

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

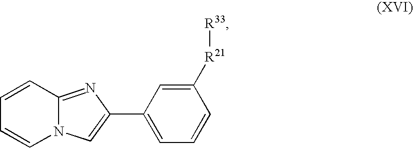

or a salt thereof wherein:

R²¹ is selected from —NR₁′—C(O)—, —N₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′, —C(O)—NR₁′—S(O)₂—, —NR₁′, —CR₁′R₁′, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, NR₁′—C(O)—CR₁′═CR₁′—CR₁′R₁′—, —NR₁′—C(—N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

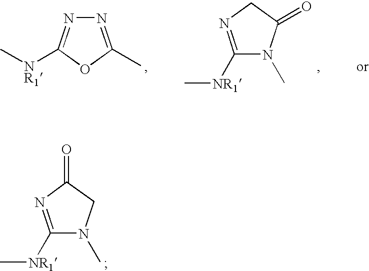

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further aspect, the invention provides [symbol] modulating compounds of Structural Formula (XVII):

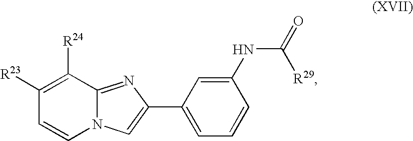

a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and

d) when R²³ is CH₃, one —O—CH₃ group at the 2 or 3 position, wherein R²⁹ is optionally additionally substituted with a solubilizing group.

In one aspect, the invention provides [symbol] modulating compounds of Structural Formula (XVIII):

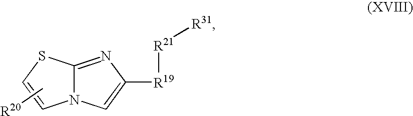

or a salt thereof, wherein

R¹⁹ is selected from:

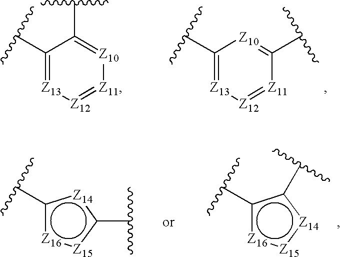

wherein:
each Z₁₀, Z₁₁, Z₁₂ and Z₁₃ is independently selected from N, CR²⁰, or CR₁′; and
each Z₁₄, Z₁₁ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

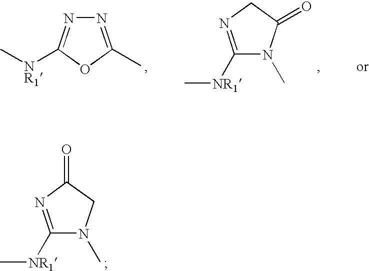

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

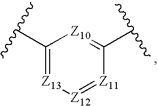

Z₁₀, Z₁₁, Z₁₂ and Z₁₃ are each CH, R²⁰ is H, and R²¹ is —NHC(O)—, R³¹ is not an optionally substituted phenyl.

In another aspect, the invention provides [symbol] modulating compounds of Structural Formula (XX):

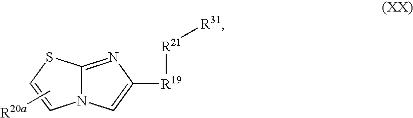

or a salt thereof, wherein

R¹⁹ is selected from:

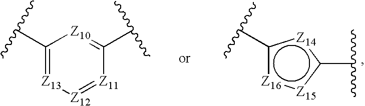

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′,
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O—CR₁′R₁′—CR₁′R₁′—, —NR₁′—C(S)—NR₁′—CR₁′R₁′—CR₁′R₁′—, —NR₁′—C(O)—O—,

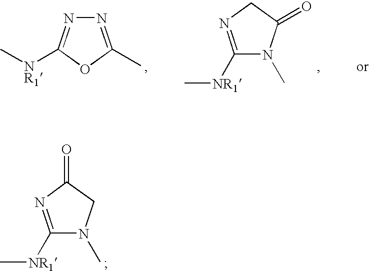

wherein
each R₁′ is independently selected from H or optionally substituted C₁-C₃ straight or branched allyl; and

R³¹ is selected from an optionally substituted monocyclic or bicyclic aryl, or an optionally substituted monocyclic or bicyclic heteroaryl, wherein when R¹⁹ is

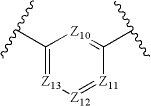

and Z₁₀, Z₁₁, Z₁₂ and Z₁₃ are each CH, R^{20a} is a solubilizing group.

In yet another aspect, the invention provides [symbol] modulating compounds of Structural Formula (XXI):

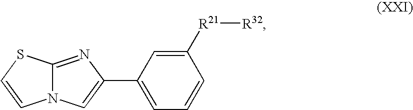

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, NR₁′—C(O)—O—,

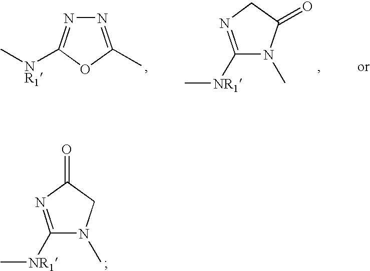

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In a further aspect, the invention provides [symbol] modulating compounds of Structural Formula (XXII):

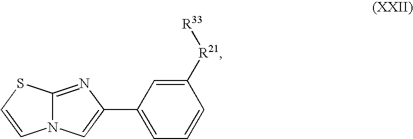

or a salt thereof; wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′C(O)—,

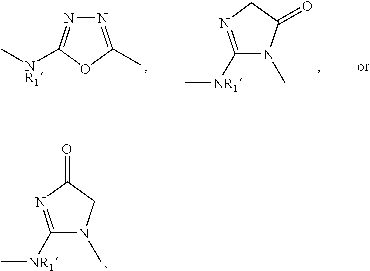

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one aspect, the invention provides [symbol] -modulating compounds of Structural Formula (XXII):

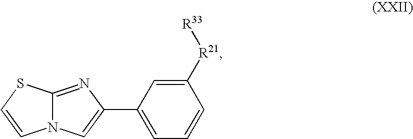

or a salt thereof wherein:

R²¹ is selected from —NH—C(O)—; or —NH—C(O)—CH₂—; and

R³³ is phenyl substituted with

a) one-N(CH₃)₂ group;

b) one CN group at the 3 position;

c) one —S(CH₃) group; or

d)

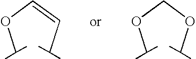

bridging the 3 and 4 positions.

In another aspect, the invention provides [symbol] modulating compounds of Structural Formula (XXIII):

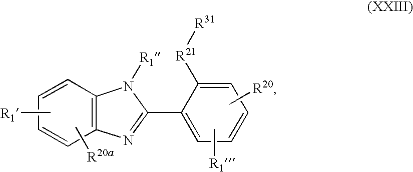

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

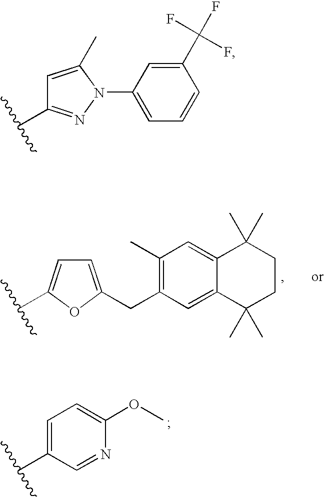

when R²¹ is —NH—C(O)— and each of R²⁰, R^{20a}, R₁′, R₁″ and R₁′″ is hydrogen, R³¹ is not

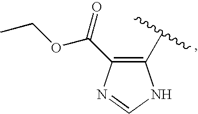

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁″ is hydrogen, R³¹ is not 2-methylaminophenyl,

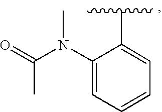

or

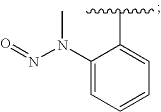

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

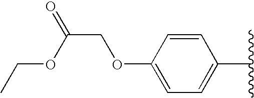

or

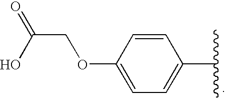

In a particular aspect, the invention provides [symbol] modulating compounds of Structural Formula (XXIII):

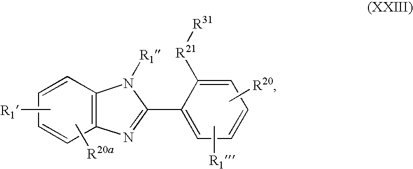

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—; —NR₁′—CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In yet another aspect, the invention provides [symbol] -modulating compounds of Structural Formula (XXIV):

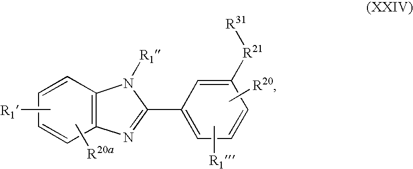

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

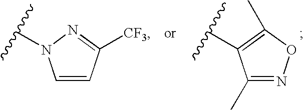

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] -modulating compounds of Structural Formula (XXIV):

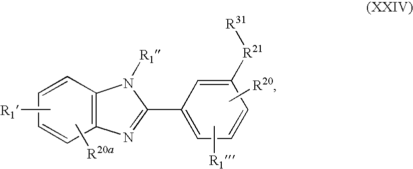

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—, —NR₁′—C(O)—CR₁′—CR₁′═—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In a further aspect, the invention provides [symbol] modulating compounds of Structural Formula (XXV):

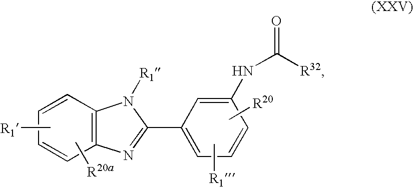

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one aspect, the invention provides [symbol] modulating compounds of Structural Formula (XXVI):

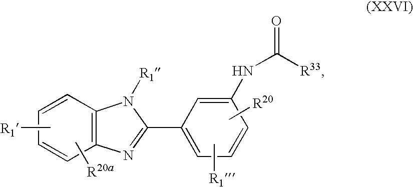

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

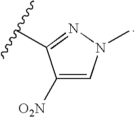

In a particular aspect, the invention provides [symbol] modulating compounds of Structural Formula (XXVI):

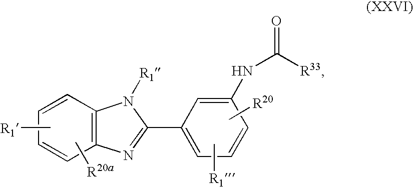

or a salt thereof, wherein:

each R²⁰ and R^{20a} is independently selected from H or a solubilizing group, wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] modulating compounds of Structural Formula (XXVII):

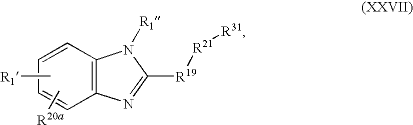

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

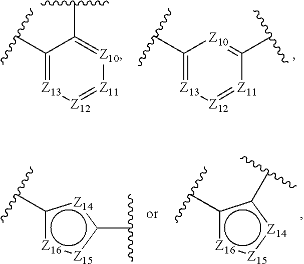

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR—CR₁′R₁′—, —NR₁′—(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(—N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

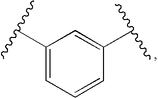

R³¹ is not unsubstituted pyridyl, 2,6-dimethoxyphenyl, 3,4,5-trimethoxyphenyl or unsubstituted furyl.

In a particular aspect, the invention provides [symbol] -modulating compounds of Structural Formula (XXVII):

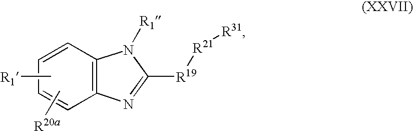

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

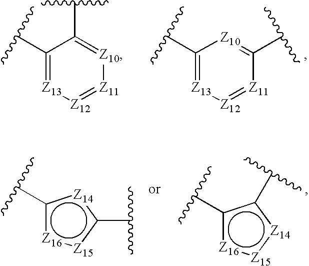

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′, —CR₁′R₁′, —C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

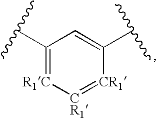

R³¹ is not 2-methylphenyl or 3,4-dimethoxyphenyl;

when R²¹ is —NH—C(O)—CH═CH—, and R¹⁹ is

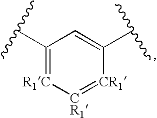

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

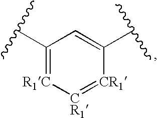

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

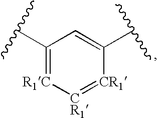

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

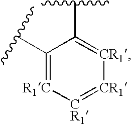

R³¹ is not 3,5-dinitrophenyl, 4-butoxyphenyl,

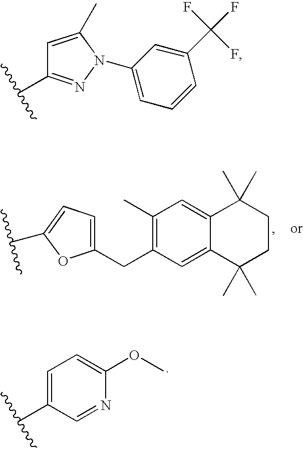

In a more particular embodiment, the invention provides [symbol] modulating compounds of Structural Formula (XXVII):

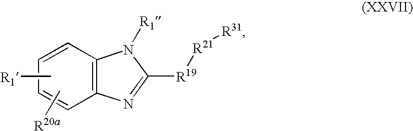

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

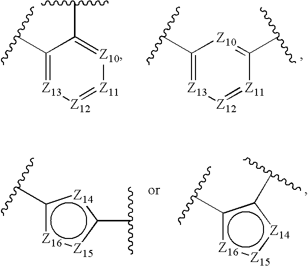

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazolyl or pyrimidinyl, R³¹ is not unsubstituted phenyl.

In yet another aspect, the invention provides compounds of Structural Formula (XXVIII):

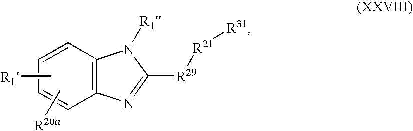

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

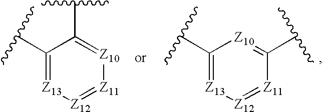

### Wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁₁—, —NR₁′—S(O)₂—CR₁′R₁—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In one aspect, the invention provides [symbol] modulating compounds of Formula (I):

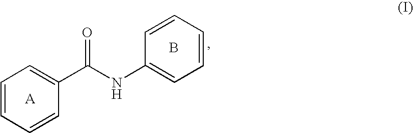

or a salt thereof where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In another aspect, the invention provides [symbol] modulating compounds of Formula (II):

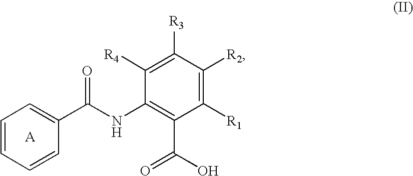

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another aspect, the invention provides [symbol] -modulating compounds of Formula (III):

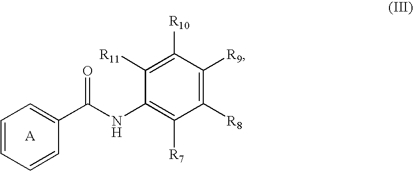

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₄, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In another aspect, the invention provides [symbol] -modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃; NR₃′R₃′; COOR₂; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₉′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a further aspect, the invention provides [symbol] -modulating compounds of Formula (IVa):

Het-L-Q-Ar′ (IVa)

or a salt thereof, where:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—,

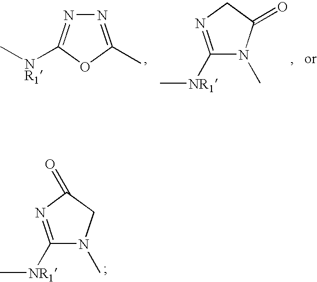

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In still yet another aspect, the invention provides [symbol] -modulating compounds of Formula (V):

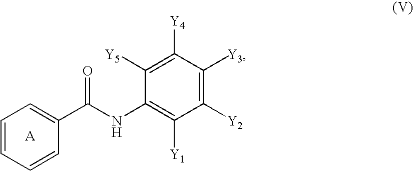

or a salt thereof, wherein:

Ring A is optionally substituted with at least one R₁′ group;

Y₁, Y₂, Y₃, Y₄, and Ys are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂R₂′; NR₂′R₃; COOR₂′; NO₂; CN; C(O)R₂′; C(O)C(O)R₂′; C(O)NR₂R₂′; OC(O)R₂′; S(O)₂R₂′; S(O)₂NR₂′R₂, NR₂′C(O)NR₂R₂′; NR₂′C(O)C(O)R₂′; NR₂′C(O)R₂; NR₂′(COOR₂); NR₂′C(O)R₅′; NR₂′S(O)₂NR₂′R₂′; NR₂′S(O)₂R₂′; NR₂S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇, COOR₇′, or S(O)₂R₇;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆; halo; haloalkyl; CF₃; OR₉′; SR₉′; NR₉′R₉; COOR₉′; NO₂; CN; C(O)R₉′; C(O)C(O)R₉′; C(O)NR₉′R₉′; S(O)₂R₉′; N(R₉)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one aspect, the invention provides [symbol] -modulating compounds of Structural Formula (VI):

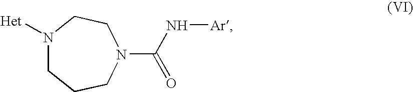

or a salt thereof, wherein:

Het is an optionally substituted heterocyclic aryl group; and

Ar′ is an optionally substituted carbocyclic or heterocyclic aryl group.

The invention also includes prodrugs and metabolites of the compounds disclosed herein.

In another aspect, the invention provides [symbol] -modulating compounds of Structural Formula (VII):

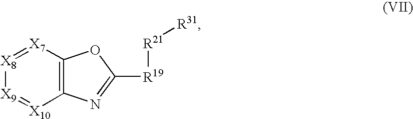

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

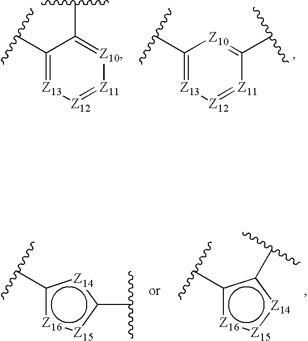

wherein:
each Z₁₀, Z₁₁, Z₁₂ and Z₁₃ is independently selected from N, CR²⁰ or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁₁—, —NR₁′—C(O)—O—,

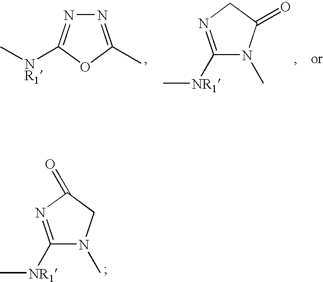

and

R³¹ is selected from an optionally substituted monocyclic or bicyclic aryl, or an optionally substituted monocyclic or bicyclic heteroaryl, with the provisos that said compound is not:

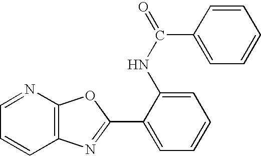

or that when R¹⁹ is

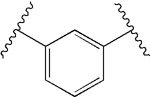

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

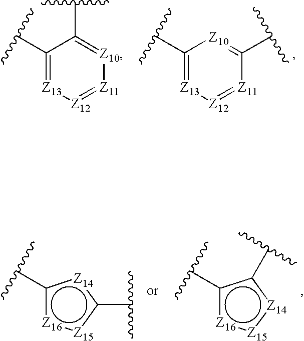

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

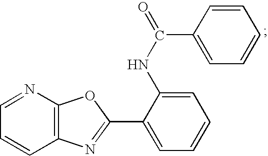

and

when X₈ and X₉ are each independently selected from CR²⁰ or CR₁′, R¹⁹ is

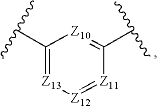

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In yet another embodiment, the invention provides [symbol] modulating compounds of Structural Formula (VIII):

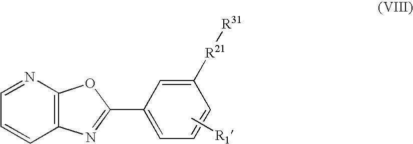

a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

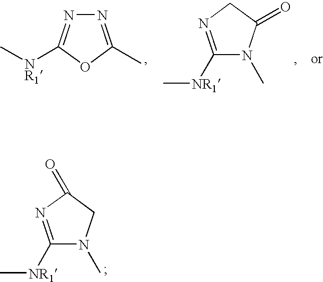

and

R³¹ is selected from an optionally substituted monocyclic or bicyclic aryl, or an optionally substituted monocyclic or bicyclic heteroaryl, with the provisos that:

when R₁′ is methyl, and R₂′ is —NH—C(O)—, R³¹ is not

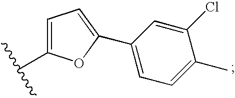

1-methoxynaphthyl; 2-methoxynaphthyl; or unsubstituted 2-thienyl;

when R₁′ is methyl, and R²¹ is —NH—C(O)—CH═CH—, R³¹ is not

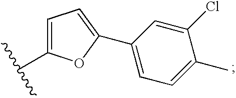

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In a further embodiment, the invention provides [symbol] -modulating compounds of Structural Formula (X):

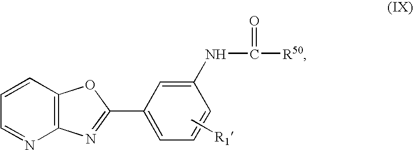

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

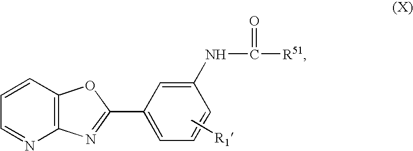

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo[b]thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In another aspect, the invention provides [symbol] -modulating compounds of Structural Formula (XI):

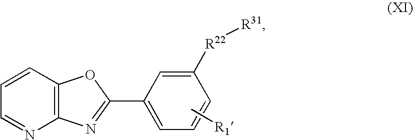

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁—═CR₁′—CR₁′R₁′—, —NR₁′—C(═N—CN)—NR₁′—, —NR₁′—C(O)—CR₁′R₁′—O—, —NR₁₁—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In yet another aspect, the invention provides [symbol] -modulating compounds of Structural Formula (XII):

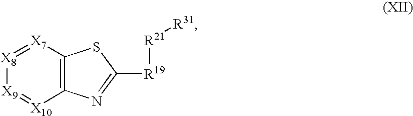

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

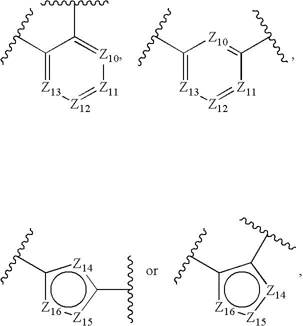

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR₂, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

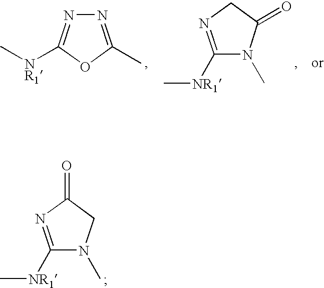

and

R³¹ is selected from an optionally substituted monocyclic or bicyclic aryl, or an optionally substituted monocyclic or bicyclic heteroaryl, with the proviso that when R¹⁹ is

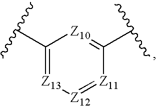

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

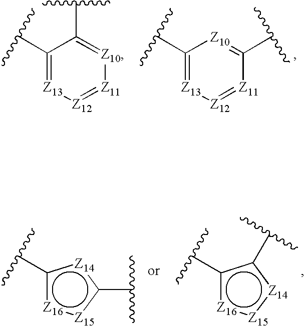

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₆ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that

when X₇ is N, R¹⁹ is

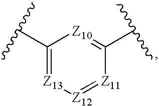

and each of Z₁₀, Z₁₁, Z₁₂ and Z₁₃ is independently selected from CR²⁰, or CR₁′, then:
a) at least one of Xs, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In a further aspect, the invention provides compounds of Structural Formula (XIII):

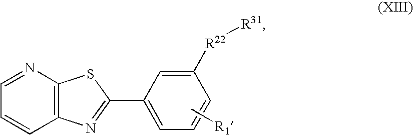

or a salt thereof; wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂, —NR₁′—, —CR₁′R₁′—, —NR₁′—C(O)—CR₁′═CR₁′—, —NR₁′—S(O)₂—NR—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁₁—CR₁′R₁′—, —NR₁′—(S)—NR₁′—CR₁′R₁′—CR₁′R₁′—, —NR₁′—C(O)—O—,

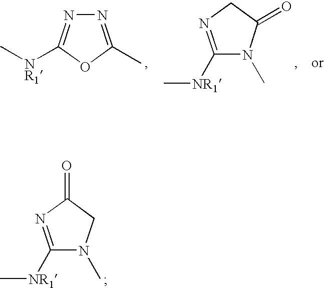

and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl; 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one aspect, the invention provides [symbol] modulating compounds of Structural Formula (XIV):

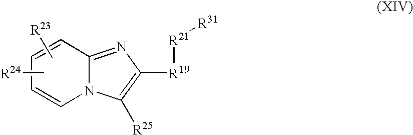

or a salt thereof, wherein:

each of R²³ and R²⁴ is independently selected from H, —CH₃ or a solubilizing group;

R²⁵ is selected from H, or a solubilizing group; and

R¹⁹ is selected from:

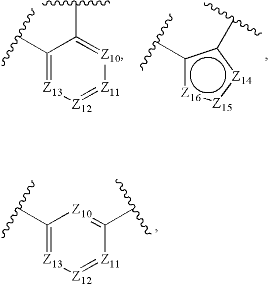

or

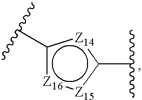

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR¹═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR¹′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

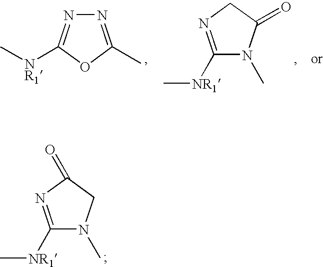

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

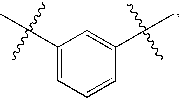

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides [symbol] -modulating compounds of Structural Formula (XV):

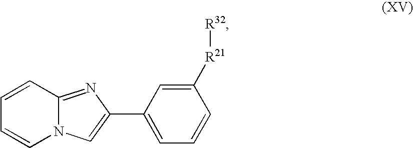

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(═N—CN)—NR₁′—, —NR₁′—C(O)—CR₁′R₁′—O—, —NR₁′—C(O)—CR₁′R₁—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′, —NR₁′—C(S)—NR₁′—CR₁′R₁′—CR₁′R₁′—, —NR₁′—C(O)—O—,

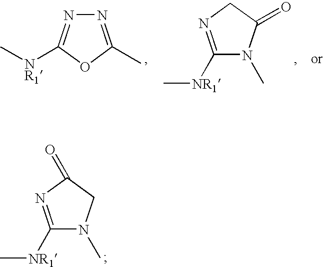

and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

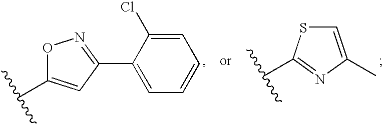

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

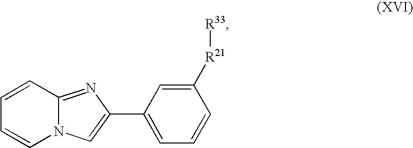

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O—NR₁′—S(O)₂—, —NR₁′—CR₁′R₁′—C(O)—NR₁′—, —CR₁′R₁′—C(O)—NR₁′—, —NR₁′—C(O)—CR₁—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C—NR—C(S)—NR₁′—CR₁′R₁′—CR₁′R₁′—, —NR₁′—C(O)—O—,

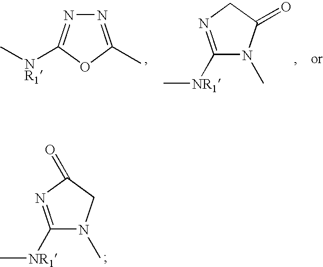

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl; 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further aspect, the invention provides [symbol] -modulating compounds of Structural Formula (XVII):

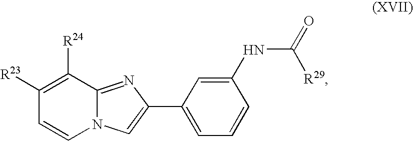

or a salt thereof; wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position, wherein R²⁹ is optionally additionally substituted with a solubilizing group.

In one aspect, the invention provides [symbol] modulating compounds of Structural Formula (XVIII):

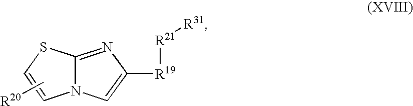

or a salt thereof, wherein

R¹⁹ is selected from:

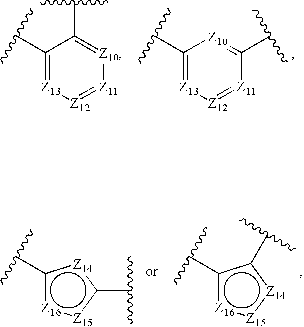

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

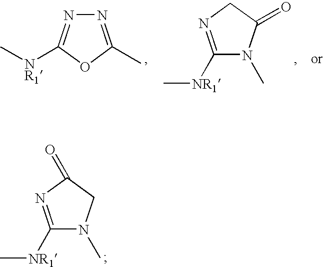

wherein each R₁′ is independently selected from H or optionally substituted C₁-C₃ straight or branched alkyl; and
R³¹ is selected from an optionally substituted monocyclic or bicyclic aryl, or an optionally substituted monocyclic or bicyclic heteroaryl, with the proviso that when R¹⁹ is

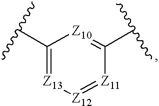

Z₁₀, Z₁₁, Z₁₂ and Z₁₃ are each CH, R²⁰ is H, and R²¹ is —NHC(O)—, R³¹ is not an optionally substituted phenyl.

In another aspect, the invention provides [symbol] -modulating compounds of Structural Formula (XX):

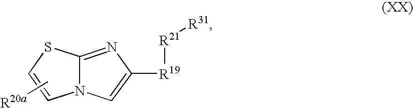

or a salt thereof wherein

R¹⁹ is selected from:

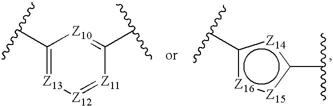

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

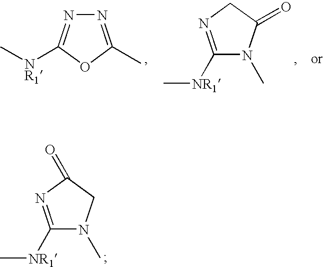

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

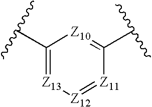

and Z₁₀, Z₁₁, Z₁₂ and Z₁₃ are each CH, R^{20a} is a solubilizing group.

In yet another aspect, the invention provides [symbol] -modulating compounds of Structural Formula (XXI):

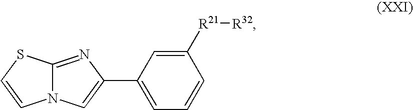

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′, —CR₁′R₁′—, —NR₁′—C(O)—O—,

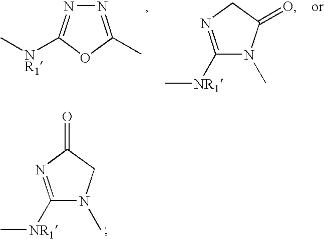

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In a further aspect, the invention provides [symbol] modulating compounds of Structural Formula (XXII):

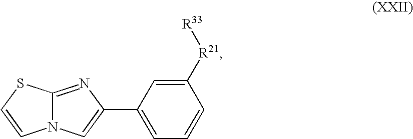

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—(S)—NR₁′—CR₁′R₁′—CR₁′R₁′—, —NR₁′—C(O)—O—,

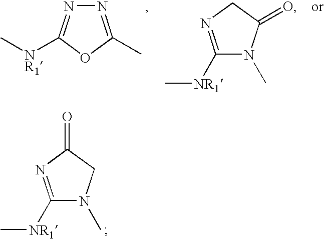

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one aspect, the invention provides [symbol] modulating compounds of Structural Formula (XXII):

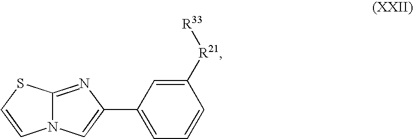

or a salt thereof wherein:

R²¹ is selected from —NH—C(O)—, or —NH—C(O)—CH₂—; and

R³³ is phenyl substituted with

a) one —N(CH₃)₂ group;

b) one CN group at the 3 position;

c) one —S(CH₃) group; or

d)

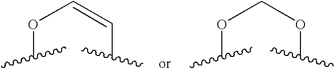

bridging the 3 and 4 positions.

In another aspect, the invention provides [symbol] -modulating compounds of Structural Formula (XXIII):

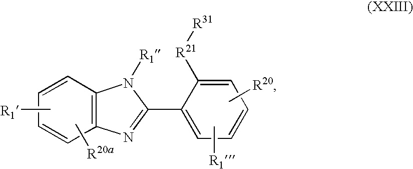

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not 3,5-nitrophenyl, 4-butoxyphenyl,

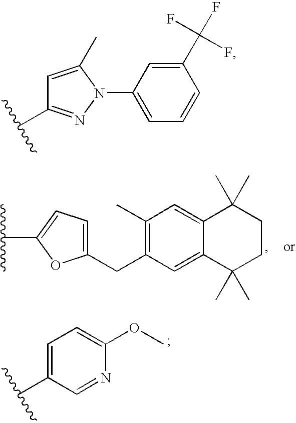

when R²¹ is —NH—C(O)— and each of R²⁰, R^{20a}, R₁′, R₁″ and R₁′″ is hydrogen, R³¹ is not

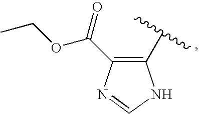

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each R²⁰, R^{20a}, R₁′ and R₁′″ is hydrogen, R³¹ is not 2-methylaminophenyl,

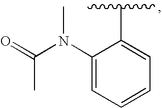

or

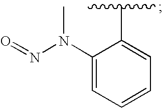

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R²⁰ is hydrogen or —CH₂—N(CH₂CH₃)₂, and each of R²⁰, R₁′, R₁″ and R₁′″ is hydro en, R³¹ is not

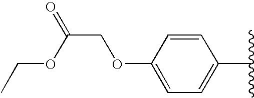

or

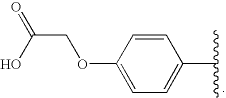

In a particular aspect, the invention provides: [symbol] -modulating compounds of Structural Formula (XXIII):

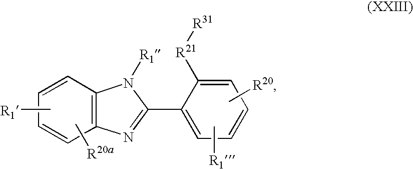

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′, —NR₁′—C(O)—CR₁′R₁′—O—, —NR₁′—C(O)—CR₁′R₁′—CR₁′R₁′—O—, —NR₁′—S(O)—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In yet another aspect, the invention provides [symbol] modulating compounds of Structural Formula (XXIV):

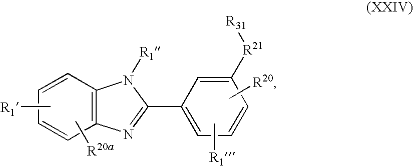

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

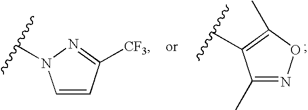

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] -modulating compounds of Structural Formula (XXIV):

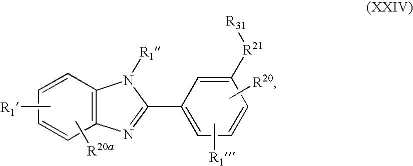

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In a further aspect, the invention provides [symbol] -modulating compounds of Structural Formula (XXV):

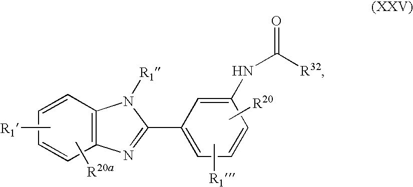

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one aspect, the invention provides [symbol] modulating compounds of Structural Formula (XXVI):

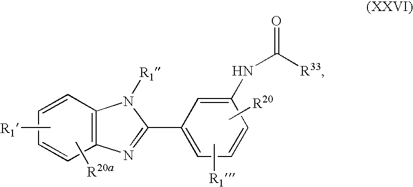

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

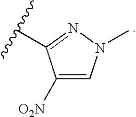

In a particular aspect, the invention provides: [symbol] -modulating compounds of Structural Formula (XXVI):

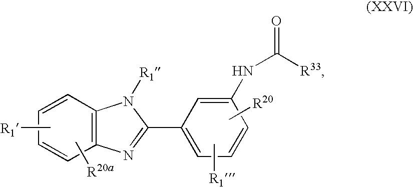

or a salt thereof, wherein:

each R²⁰ and R^{20a} is independently selected from H or a solubilizing group, wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] modulating compounds of Structural Formula (XXVII):

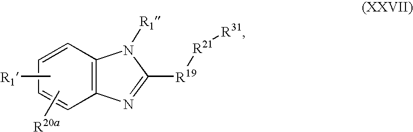

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

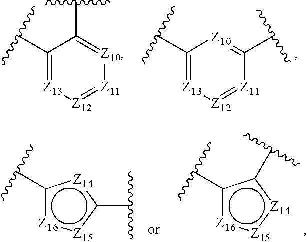

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

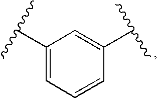

R³¹ is not unsubstituted pyridyl, 2,6-dimethoxyphenyl, 3,4,5-trimethoxyphenyl or unsubstituted furyl.

In a particular aspect, the invention provides [symbol] -modulating compounds of Structural Formula (XXVII):

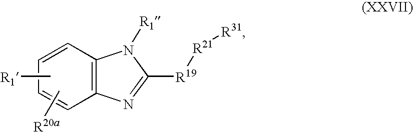

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

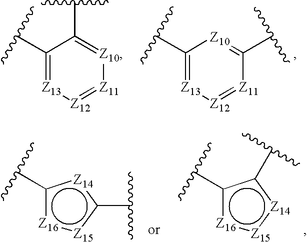

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)_{z}—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

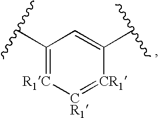

R³¹ is not 2-methylphenyl or 3,4-dimethoxyphenyl;

when R²¹ is —NH—C(O)—CH═CH—, and R¹⁹ is

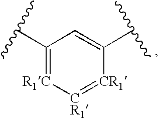

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

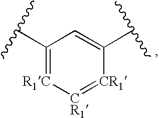

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

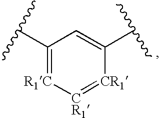

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

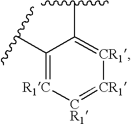

R³¹ is not 3,5-dinitrophenyl, 4-butoxyphenyl,

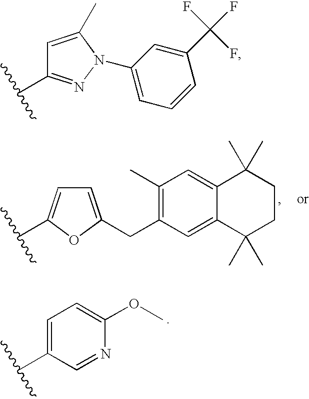

In a more particular embodiment, the invention provides [symbol] -modulating compounds of Structural Formula (XXVII):

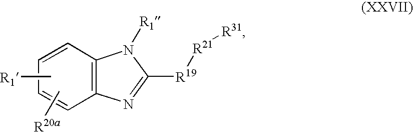

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

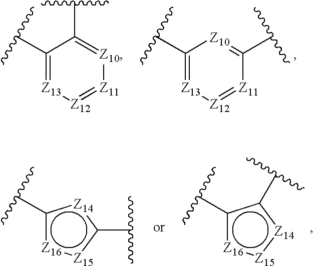

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R²⁰ is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazolyl or pyrimidinyl, R³¹ is not unsubstituted phenyl.

In yet another aspect, the invention provides compounds of Structural Formula (XXVII):

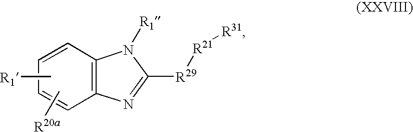

or a salt thereof wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

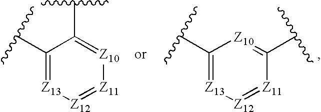

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In one aspect, the invention provides [symbol] modulating compounds of Formula (I):

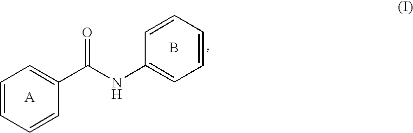

or a salt thereof, where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In another aspect, the invention provides [symbol] -modulating compounds of Formula (II):

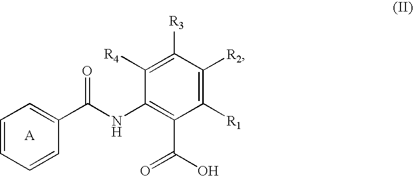

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another aspect, the invention provides: [symbol] -modulating compounds of Formula (III):

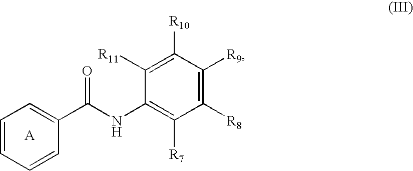

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl-group; and

n is 1 or 2.

In another aspect, the invention provides [symbol] -modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂; S(O)₂R₂′; S(O)₂NR₂′R₂; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur, oxygen; CF₃; haloalkyl; SR₂′, OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′ or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl; each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a further aspect, the invention provides [symbol] -modulating compounds of Formula (IVa):

Het-L-Q-Ar′ (IVa)

or a salt thereof, where:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—,

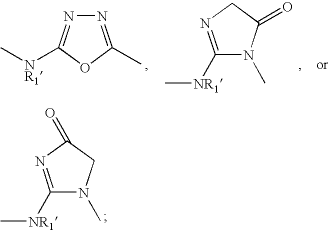

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation; and Ar′ is optionally substituted phenyl; then Q is not —NH—C(O)—.

In still yet another aspect, the invention provides [symbol] modulating compounds of Formula (V):

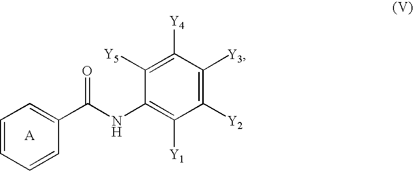

or a salt thereof, wherein:

Ring A is optionally substituted with at least one R₁′ group;

Y₁, Y₂, Y₃, Y₄, and Y₅ are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂; NR₂′C(O)NR₂′R₂′; —NR₂′C(O)C(O)C(O)R₂′; NR₂′C(O)R₂′; NR₂′(COOR₂′); NR₂′C(O)R₅′; NR₂′S(O)₂NR₂′R₂′; NR₂S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₁′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and each aryl is independently a 5- to 7=membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one aspect, the invention provides [symbol] -modulating compounds of Structural Formula (VI):

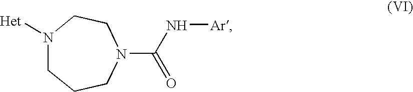

or a salt thereof, wherein:

Het is an optionally substituted heterocyclic aryl group; and

Ar′ is an optionally substituted carbocyclic or heterocyclic aryl group.

The invention also includes prodrugs and metabolites of the compounds disclosed herein.

In another aspect, the invention provides [symbol] -modulating compounds of Structural Formula (VII):

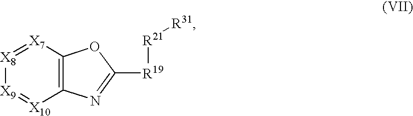

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

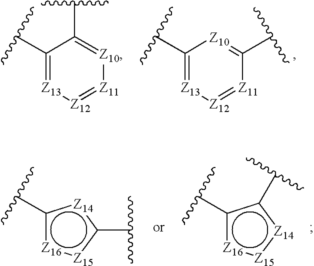

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—, —CR₁′R₁′—, —NR₁′—C(O)—O—,

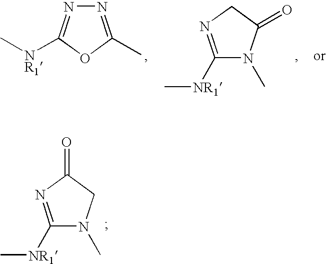

and

R³¹ is selected from an optionally substituted monocyclic or bicyclic aryl, or an optionally substituted monocyclic or bicyclic heteroaryl, with the provisos that said compound is not.

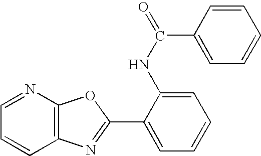

that when R¹⁹ is

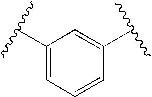

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

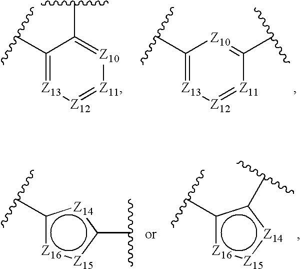

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂, or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁—)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—; —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

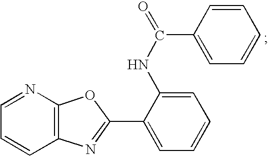

and

when X₈ and X₉ are each independently selected from CR²⁰ or CR₁′, R¹⁹ is

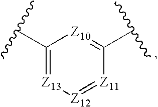

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In yet another embodiment, the invention provides [symbol] modulating compounds of Structural Formula (VIII):

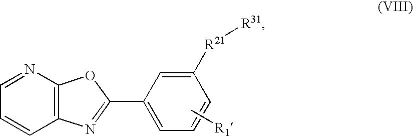

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

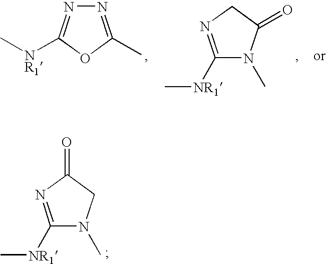

and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

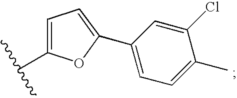

1-methoxynaphthyl; 2-methoxynaphthyl; or unsubstituted 2-thienyl;

when R₁′ is methyl, and R²¹ is —NH—C(O)—CH═CH—, R³¹ is not

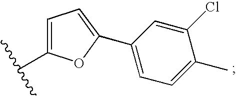

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In a further embodiment, the invention provides [symbol] -modulating compounds of Structural Formula (IX):

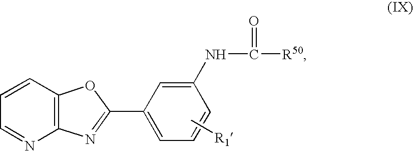

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

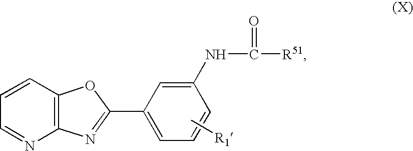

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo[b]thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In another aspect, the invention provides [symbol] modulating compounds of Structural Formula (XI):

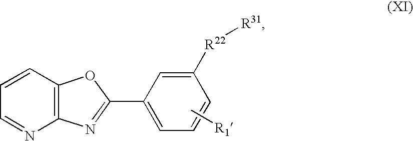

or a salt thereof, wherein:

R₁′ is selected from H or optionally substituted C₁-C₃ straight or branched alkyl;

R₂₂ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—; —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In yet another aspect, the invention provides [symbol] -modulating compounds of Structural Formula (XII):

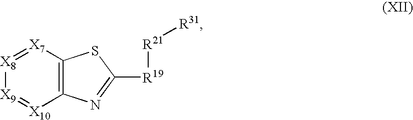

or a salt thereof, herein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

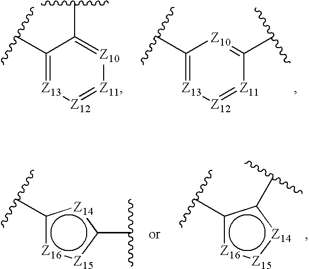

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁—CR₁′R₁′—, —NR₁′—C(O)—O—,

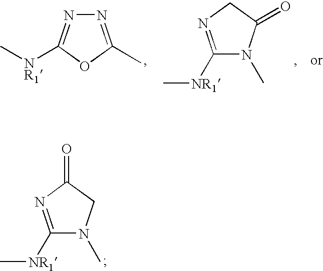

and

R³¹ is selected from an optionally substituted monocyclic or bicyclic aryl, or an optionally substituted monocyclic or bicyclic heteroaryl, with the proviso that when R¹⁹ is

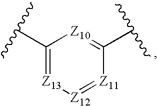

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

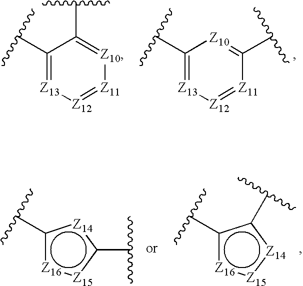

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

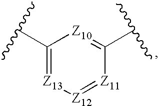

and each of Z₁₀, Z₁₁, Z₁₂ and Z₁₃ is independently selected from CR²⁰, or CR₁′, then:
a) at least one of X₈, X₉ or X_{f} o is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In a further aspect, the invention provides compounds of Structural Formula (XIII):

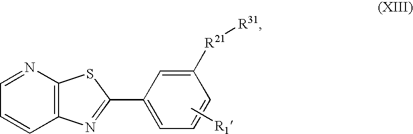

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

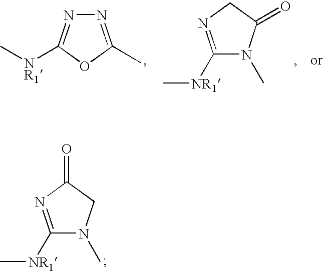

and

R³¹ is selected form an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one aspect, the invention provides [symbol] -modulating compounds of Structural Formula (XIV):

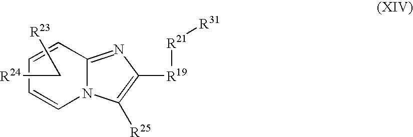

or a salt thereof, wherein:

each of R²³ and R²⁴ is independently selected from H, —CH₃ or a solubilizing group;

R²⁵ is selected from H, or a solubilizing group; and

R¹⁹ is selected from:

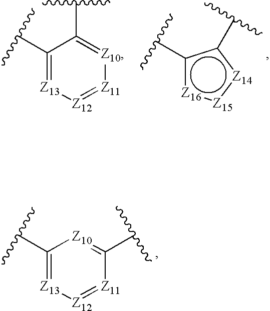

or

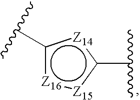

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, ZS and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

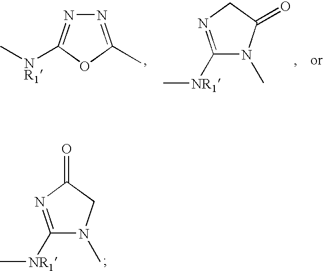

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

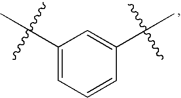

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides [symbol] -modulating compounds of Structural Formula (XV):

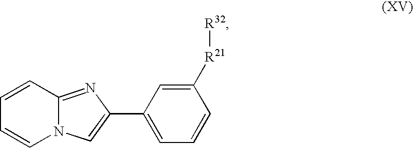

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′R₁′—, —NR₁′—C(O)—O—,

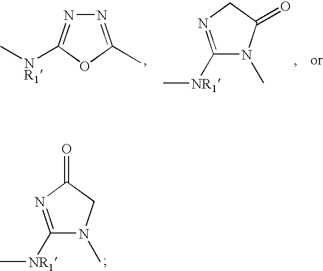

and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

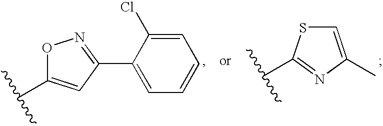

when R²¹ is —NR₁′—S(O)₂—, R³² is not unsubstituted 2-thienyl or unsubstituted naphthyl.

In yet another aspect, the invention provides [symbol] modulating compounds of Structural Formula (XVI):

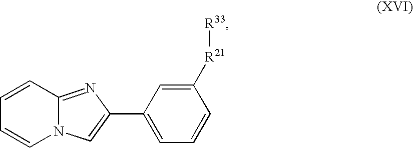

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

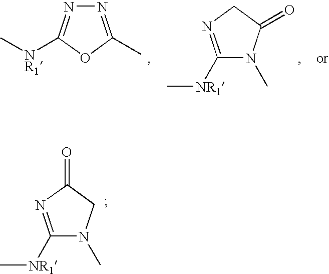

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further aspect, the invention provides [symbol] -modulating compounds of Structural Formula (XVII):

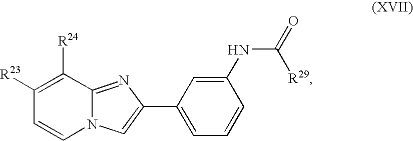

or a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position, wherein R²⁹ is optionally additionally substituted with a solubilizing group.

In one aspect, the invention provides [symbol] -modulating compounds of Structural Formula (XVIII):

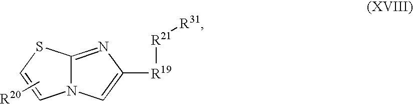

or a salt thereof, wherein

R¹⁹ is selected from:

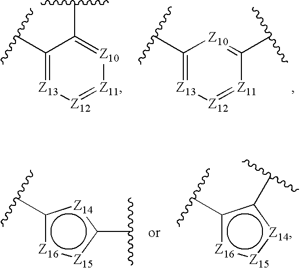

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁₁—, —NR₁′—C(S)—NR₁′—CR₁′R₁′—CR₁′R₁′—, —NR₁′—C(O)—O—,

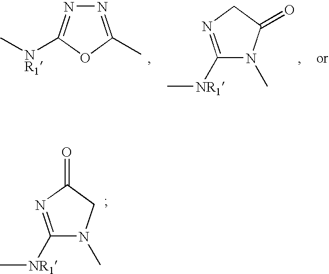

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

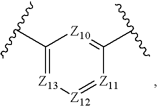

Z₁₀, Z₁₁, Z₁₂ and Z₁₃ are each CH, R²⁰ is H, and R²¹ is —NHC(O)—, R³¹ is not an optionally substituted phenyl.

In another aspect, the invention provides [symbol] -modulating compounds of Structural Formula (XX):

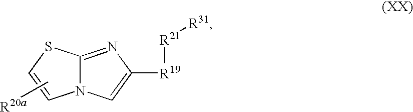

or a salt thereof, wherein

R¹⁹ is selected from:

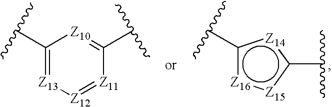

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C1-C3 straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′-C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁—CR₁′R₁′—, —NR₁′—C(O)—CR₁′R₁′—; —NR₁′—C(O)—CR₁′R₁—CR₁′R₁—, —NR₁′—C(S)—NR₁′—CR₁′R₁′—CR₁′R₁′—, —NR₁′—C(O)—O—,

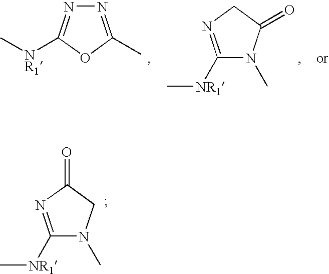

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

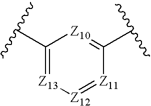

and Z₁₀, Z₁₁, Z₁₂ and Z₁₃ are each CH, R^{20a} is a solubilizing group.

In yet another aspect, the invention provides [symbol] modulating compounds of Structural Formula (XXI):

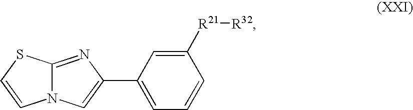

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

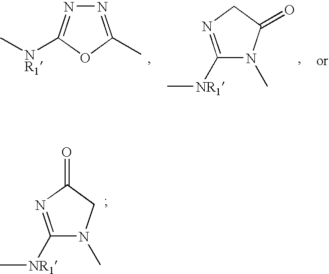

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In a further aspect, the invention provides [symbol] -modulating compounds of Structural Formula (XXII):

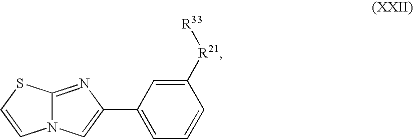

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

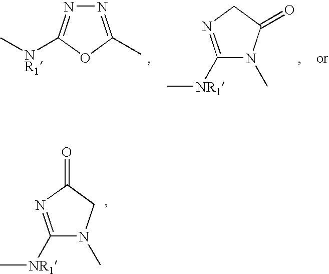

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one aspect, the invention provides [symbol] modulating compounds of Structural Formula (XXII):

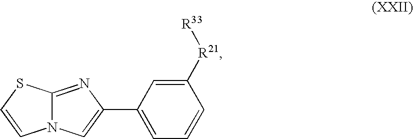

or a salt thereof wherein:

R²¹ is selected from —NH—C(O)—, or —NH—C(O)—CH₂—; and

R³³ is phenyl substituted with

a) one —N(CH₃)₂ group;

b) one CN group at the 3 position;

c) one —S(CH₃) group; or

d)

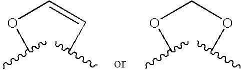

bridging the 3 and 4 positions.

In another aspect, the invention provides— [symbol] modulating compounds of Structural Formula (XXIII):

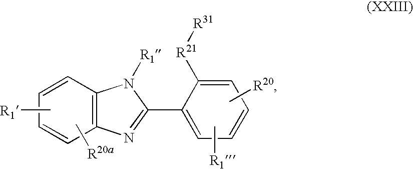

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl

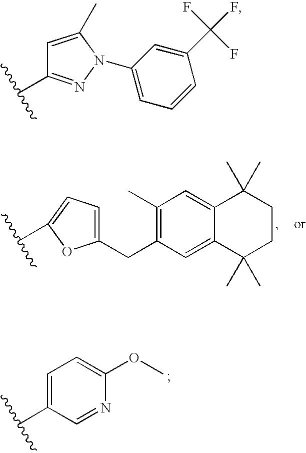

when R²¹ is —NH—C(O)— and each of R²⁰, R^{20a}, R₁′, R₁″ and R₁′″ is hydrogen, R³¹ is not

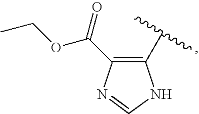

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁′″ is hydrogen, R³¹ is not 2-methylaminophenyl,

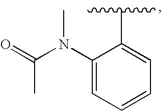

or

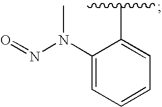

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

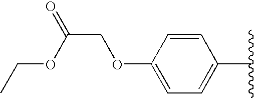

or

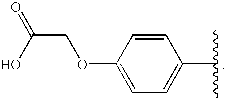

In a particular aspect, the invention provides [symbol] -modulating compounds of Structural Formula (XXIII):

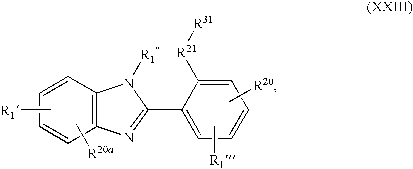

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In yet another aspect, the invention provides [symbol] modulating compounds of Structural Formula (XXIV):

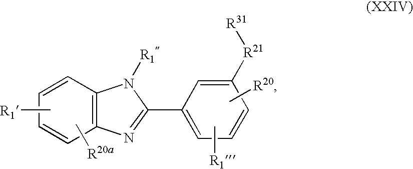

or a salt thereof, wherein:

each R²⁰ and R^{20a} is independently selected from H or a solubilizing group;

each R₁′, R¹″ and R¹′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen; and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

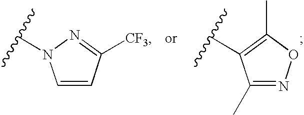

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] -modulating compounds of Structural Formula (XXIV):

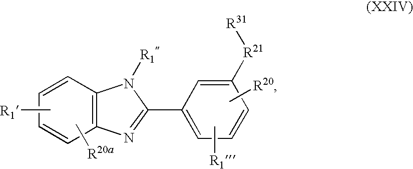

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In a further aspect, the invention provides [symbol] modulating compounds of Structural Formula (XXV):

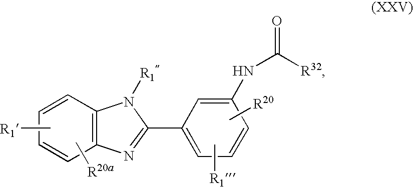

or a salt hereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one aspect, the invention provides [symbol] modulating compounds of Structural Formula (XXVI):

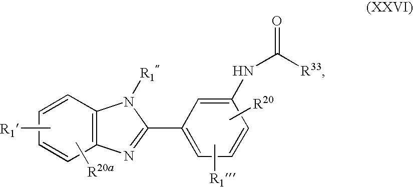

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

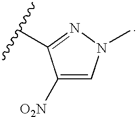

In a particular aspect, the invention provides [symbol] modulating compounds of Structural Formula (XXVI):

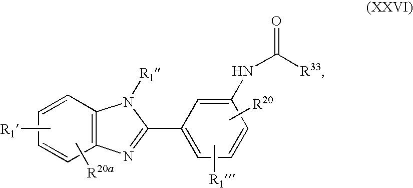

or a salt thereof, wherein:

each R²⁰ and R^{20a} is independently selected from H or a solubilizing group, wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] modulating compounds of Structural Formula (XXVII):

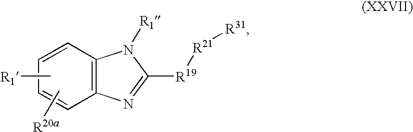

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

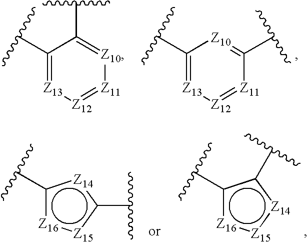

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR¹′R₁′—NR₁′—, —NR₁′—C(═NR₁′—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

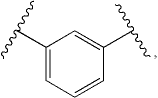

R³¹ is not unsubstituted pyridyl, 2,6-dimethoxyphenyl, 3,4,5-trimethoxyphenyl or unsubstituted furyl.

In a particular aspect, the invention provides [symbol] -modulating compounds of Structural Formula (XXVII):

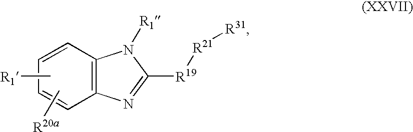

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

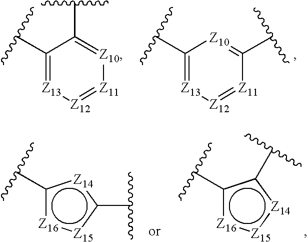

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

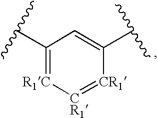

R³¹ is not 2-methylphenyl or 3,4-dimethoxyphenyl;

when R²¹ is —NH—C(O)—CH═CH—, and R¹⁹ is

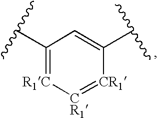

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

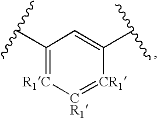

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

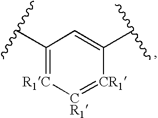

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

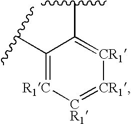

R³¹ is not 3,5-dinitrophenyl, 4-butoxyphenyl,

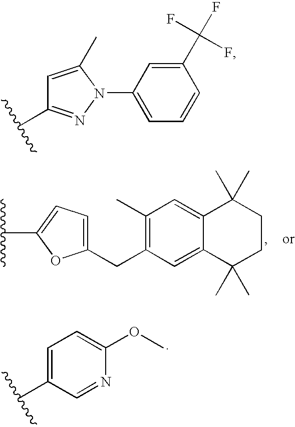

In a more particular embodiment, the invention provides: [symbol] -modulating compounds of Structural Formula (XXVII):

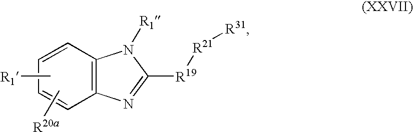

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

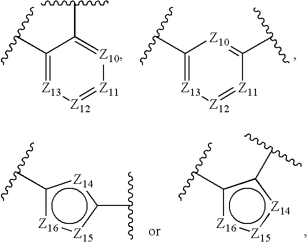

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and

when R²¹ is —NH—C(O)— and R¹⁹ is thiazolyl of pyrimidinyl, R³¹ is not unsubstituted phenyl.

In yet another aspect, the invention provides compounds of Structural Formula (XXVIII):

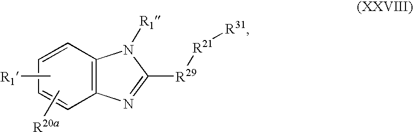

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

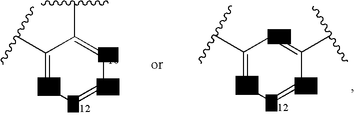

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′—CR₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In one aspect, the invention provides novel [symbol] -modulating compounds of Formula (I):

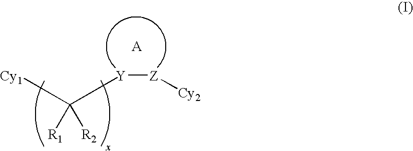

or a salt thereof, where:

Ring A is an optionally substituted, aliphatic, carbocyclic or heterocyclic ring;

R₁ and R₂ are independently halogen, —OR₄, CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂, or R₁ and R₂ taken together are ═O, ═S or ═N;

R₄ and R₅ are independently —H, a substituted or unsubstituted alkyl group, a substituted or unsubstituted aryl group or a substituted or unsubstituted non-aromatic heterocyclic group;

Cy₁ and Cy₂ are independently cyclic groups optionally fused to Ring A;

Y and Z are independently CH, N, O or S, provided that O and S are not adjacent to another O or S;

n is 1 or 2; and

x is 0 or 1.

In another aspect, the invention provides novel [symbol] -modulating compounds of Formula (III):

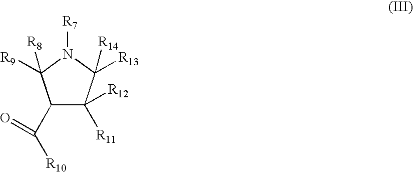

or a salt thereof, here:

R₄ and R₅ are independently —H, a substituted or unsubstituted alkyl group, a substituted or unsubstituted aryl group or a substituted or unsubstituted non-aromatic heterocyclic group;

R₇ is —H, a substituted or unsubstituted alkyl group, a substituted or unsubstituted aryl group or a substituted or unsubstituted non-aromatic heterocyclic group;

R₈ is —H;

R₉ is a substituted or unsubstituted aryl group;

R₁₀ is a substituted or unsubstituted aryl group;

R₁₁ is —H;

R₁₂ is a substituted or unsubstituted aryl group;

R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)R₄, —S(O)OR₄, —S(O)NR₄R₅, —NR₄R₅, —NR₄C(O)OR₅, —NR₄C(O)R₅ and —NO₂; and

n is 1 or 2.

The invention also includes salts, prodrugs and metabolites of the compounds disclosed herein.

Also provided are pharmaceutical compositions comprising one or more compounds of Formulas (I)-(IV) or a salt, prodrug or metabolite thereof.

In another aspect, the invention provides methods for using [symbol] -modulating compounds, or compostions comprising [symbol] modulating compounds.

In one aspect, the invention provides novel [symbol] -modulating compounds of Formula (I):

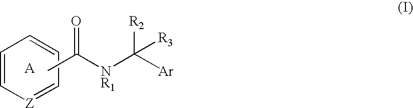

or a salt thereof, where:

Ring A is optionally substituted with one or more substituents in addition to the substituent indicated in Structural Formula (I)

Ar is a substituted or unsubstituted aryl group;

R₁ is —H, a substituted or unsubstituted alkyl group, a substituted or unsubstituted alkenyl group, a substituted or unsubstituted aryl group or a substituted or unsubstituted non-aromatic heterocyclic group, typically —H, a substituted or unsubstituted alkyl group, a substituted or unsubstituted aryl group or a substituted or unsubstituted non-aromatic heterocyclic group;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR⁴, —SR₄, —OSO₃H, —S(O)ₙR₄, —S(O)ₙOR₄, —S(O)NR₄R₅, —NR₄R₅, —NR₄C(O)OR₅, —NR₄C(O)R₅ and —NO₂;

R₄ and R₅ are independently —H, a substituted or unsubstituted alkyl group, a substituted or unsubstituted aryl group or a substituted or unsubstituted non-aromatic heterocyclic group;

Z is CH or N; and

n is 1 or 2.

In another aspect, the invention provides novel [symbol] modulating compounds of Formula (II):

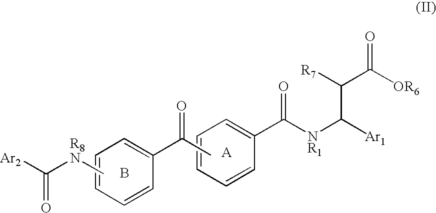

or a salt thereof, where:

Rings A and B are optionally substituted with one or more substituents in addition to the substituents indicated in Structural Formula (II)

Ar₁ and Ar₂ are independently a substituted or unsubstituted aryl group;

R₁, R₆ and R₈ are independently —H, a substituted or unsubstituted alkyl group, a substituted or unsubstituted aryl group or a substituted or unsubstituted non-aromatic heterocyclic group;

R₄ and R₅ are independently —H, a substituted or unsubstituted alkyl group, a substituted or unsubstituted aryl group or a substituted or unsubstituted non-aromatic heterocyclic group;

R₇ is selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and

n is 1 or 2.

In yet another aspect, the invention provides novel [symbol] -modulating compounds of Structural Formula (IVa):

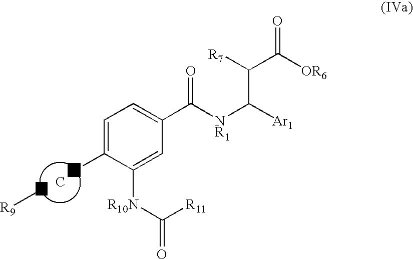

or a salt thereof, wherein:

Ring C is a substituted or unsubstituted 5-, 6- or 7-membered ring;

Ar₁ is a substituted or unsubstituted aryl group;

R₁, R₆ and R₁₀ are independently —H, a substituted or unsubstituted alkyl group, a substituted or unsubstituted aryl group or a substituted or unsubstituted non-aromatic heterocyclic group;

R₄ and R₅ are independently —H, a substituted or unsubstituted alkyl group, a substituted or unsubstituted aryl group or a substituted or unsubstituted non-aromatic heterocyclic group;

R₇ is selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄; —OCOR₄, —OCO₂₄, —C(O)NR₄R₅, —OC(O)NR₄R₅, —C(O)R₄, —COR₄, —SR₄, —OSO₃H, —S(O)ₙR₄, —S(O)ₙOR₄, —S(O)NR₄R₅, —NR₄R₅, —NR₄C(O)OR₅, —NR₄C(O)R₅ and —NO₂;

R₉ is —H, a substituted or unsubstituted acyl group, a substituted or unsubstituted aminocarbonyl group, a substituted or unsubstituted alkyl group, a substituted or unsubstituted aryl group or a substituted or unsubstituted non-aromatic heterocyclic group;

R₁₁ is —H, a substituted or unsubstituted alkyl group, a substituted or unsubstituted aryl group or a substituted or unsubstituted non-aromatic heterocyclic group;

X and Y are independently CH or N; and

n is 1 or 2.

A particular group of compounds represented by Structural Formula (IVa) are represented by Structural Formula (IVb)

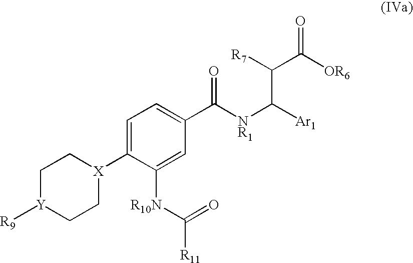

or a salt thereof, where:

Ar₁ is a substituted or unsubstituted aryl group;

R₁, R₆ and R₁₀ are independently —H, a substituted or unsubstituted alkyl group, a substituted or unsubstituted aryl group or a substituted or unsubstituted non-aromatic heterocyclic group;

R₄ and R₅ are independently —H, a substituted or unsubstituted alkyl group, a substituted or unsubstituted aryl group or a substituted or unsubstituted non-aromatic heterocyclic group;

R₇ is selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙN₄R₅, —NR₄R₅, —NR₄C(O)OR₅, —NR₄C(O)R₅ and —NO₂;

R₉ is —H, a substituted or unsubstituted acyl group, a substituted or unsubstituted aminocarbonyl group, a substituted or unsubstituted alkyl group, a substituted or unsubstituted aryl group or a substituted or unsubstituted non-aromatic heterocyclic group;

R₁₁ is —H, a substituted or unsubstituted alkyl group, a substituted or unsubstituted aryl group or a substituted or unsubstituted non-aromatic heterocyclic group;

X and Y are independently CH or N; and

n is 1 or 2.

The invention also includes salts, prodrugs and metabolites of the compounds disclosed herein.

In one aspect, the invention provides novel [symbol] -modulating compounds of Formula (I):

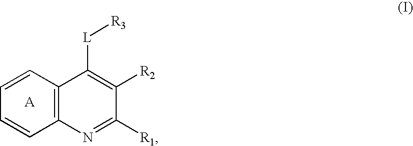

or a salt thereof, where, as valence permits:

Ring A is optionally substituted;

R₁ and R₂ are independently selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₂H, —S(O)ₙR₄, —S(O)ₙOR₄, —S(O)ₙNR₄R₅, —NR₄R₅, —NR₄C(O)ORS, —NR₄C(O)R₅ and —NO₂, or R₁ and R₂ taken together with the atoms to which they are attached form an optionally substituted ring;

L is selected from —CH═CH—C(O)—, —CH₂—N(R₄)—C(O)—, —C(O)—CH₂—, —C(O)NR₄—, —C(O)—N(R₄)—C(O)—, —C(O)—N(R₄)—N(R₅)—, —C(O)—N(R₄)—N(R₅)—C(O)—, —CH₂—N(R₄)—N(R₅)—, —N(R₄)—S(O)₂—, S(O)₂—N(R₄)—, —N(R₅)—C(O)—, —N(R₄—N(R₅)—CH₂, —N(R₄)—N(R₅)— or

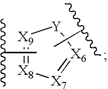

R₃, R₄ and R₅ are, independently for each occurrence, —H, a substituted or unsubstituted alkyl group, a substituted or unsubstituted aryl group or a substituted or unsubstituted non-aromatic heterocyclic group;

Y is selected from O, S, or NR₄;

each of X₆, X₇, X₈ and X₉ is independently selected from CR₇, C, or N, wherein at least two of X₆, X₇, X₈ or X₉ are not N;

each R₇ is independently selected from H or (C₁-C₃)-straight or branched alkyl; and

n is 1 or 2.

In another aspect, the invention provides novel [symbol] modulating compounds of Formula (II):

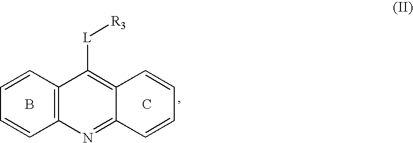

or a salt thereof, where:

Rings B and C are independently optionally substituted;

L is —NR₄R₅—, —C(O)O—, —C(O)NR₄—, —NR₄C(O)—, —NR₄—NR₅—C(O)—, —C(O)—NR₄—NR₅— or —CHR₄═CHR₅—; and

R₃, R₄ and R₅ are independently —H, a substituted or unsubstituted alkyl group, a substituted or unsubstituted aryl group or a substituted or unsubstituted non-aromatic heterocyclic group.

In yet another aspect, the invention provides novel: [symbol] modulating compounds of Formula (III):

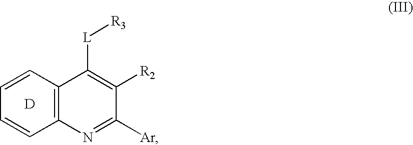

or a salt thereof, where, as valence permits:

Ring D is optionally substituted;

Ar is a substituted or unsubstituted aryl group;

R₂ is selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

L is —C(O)NR₄—, —NR₄C(O)—, —NR₄—NR₅—C(O)—, —C(O)—NR₄—NR₅— or —CHR₄═CHR₅—;

R₃, R₄ and R₅ are independently —H, a substituted or unsubstituted alkyl group, a substituted or unsubstituted aryl group or a substituted or unsubstituted non-aromatic heterocyclic group; and

n is 1 or 2.

Prodrugs and metabolites of the preceding compounds are also included in the invention.

In a further aspect, the invention provides sirtuin- [symbol] modulating compounds represented by Structural Formula (V):

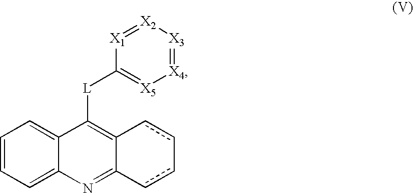

wherein, as valence permits:

each of X₁, X₂, X₃, X₄ and X₅ is independently selected from N or CR₆, wherein no more than two of X₁, X₂, X₃, X₄ or Xs are N;

each R₆ is independently selected from H, —OCH₃, —CH₃, or —CF₃;

L is selected from —CH═CH—C(O)—, —CH₂—N(R₄)—C(O)—, —C(O)—CH₂—, —C(O)—N(R₄)—, —C(O)—N(R₄)—CH₂—, —C(O)—N(R₄)—CH₂—CH₂—, —C(O)—N(R₄)—C(O)—, —C(O)—N(R₄)—N(R₅)—, —CH₂—N(R₄)—N(R₅)—, —N(R₄)—S(O)₂—, —S(O)₂—N(R₄)—, —N(R₄)—N(R₅)—C(O)—, —C(O)—N(R₄)—N(R₅)—C(O)—, —N(R₄)—N(R₅)—CH₂, —N(R₄)—N(R₅)—,

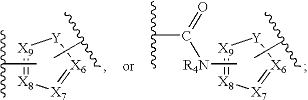

each of R₄ and R₅ is independently selected from H or CH₃;

Y is selected from O, S, or NR₄;

each of X₆, X₇, X₈ and X₉ is independently selected from CR₇, C, or N, wherein at least two of X₆, X₇, X₈ or X₉ are not N;

each R₇ is independently selected from H or (C₁-C₃)-straight or branched alkyl; and

the hashed bonds are either simultaneously present or simultaneously absent.

In one aspect, the invention provides novel [symbol] -modulating compounds of Formula (I):

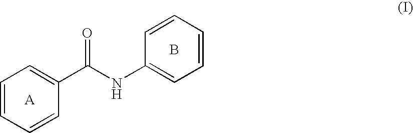

or a salt thereof, where:

Ring A is optionally substituted; and

Ring B is substituted with at least one carboxy or polycyclic aryl group.

In another aspect, the invention provides novel [symbol] -modulating compounds of Formula (II):

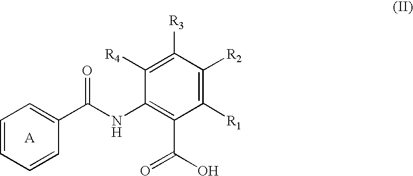

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)OR₅, —S(O)NR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another aspect, the invention provides novel [symbol] -modulating compounds of Formula (III):

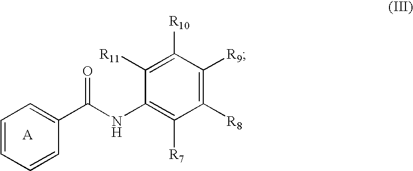

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅; —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)Rₛ, —S(O)ₙOR₅, —S(O)NR₅R₆, —NR₅R₆, —NR₅C(O)OR₆, —NR₅C(O)R₆ and —NO₂;

R₈ is a polycyclic aryl group; and

n is 1 or 2.

In one aspect, the invention provides novel [symbol] -modulating compounds of Formula (I):

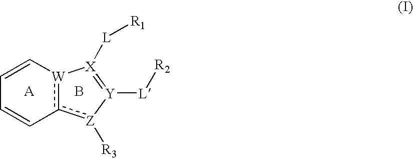

or a salt thereof, where, as valence permits:

Ring A is optionally substituted;

L is absent, substituted or unsubstituted phenylene, substituted or unsubstituted —O-phenylene, substituted or unsubstituted thienylene, substituted or unsubstituted pyrazolylene, substituted or unsubstituted benzothiazolylene, —NR₄—, —C(O)O—, —C(O)NR₄—, —NR₄C(O)—, —NR₄—C(O)—NR₅—, —S—, —CHR₆═CHR₇— or —CHR₆—C(O)—;

L′ is absent, substituted or unsubstituted phenylene, substituted or unsubstituted —O-phenylene, substituted or unsubstituted thienylene, substituted or unsubstituted pyrazolylene, substituted or unsubstituted benzothiazolylene, substituted or unsubstituted indenedionylene, —C(O)O—, —C(O)NR₄—, —NR₄C(O)—, —NR₄—C(O)—NR₅—, —S—, —CHR₆═CHR₇ or —CHR₈—C(O)—, provided that at least one of L and L′ is substituted or unsubstituted phenylene, substituted or unsubstituted —O-phenylene, substituted or unsubstituted thienylene, substituted or unsubstituted pyrazolylene, substituted or unsubstituted benzothiazolylene, substituted or unsubstituted indenedionylene, —NR₄—, —C(O)O—, —C(O)NR₄—, —NR₄C(O)—, —NR₄—C(O)—NR₅—, —S—, —CHR₆═CHR₇— or —CHR₈—C(O)—;

R₁ is absent, —H, —NR₄R₅, —N₄C(O)R₅, —OR₅, naphthyl or a heterocyclic group, provided that L and R₁ are not both absent unless X is N;

R₂ is —H, unsubstituted alkyl, —NR₄R₅, —NR₄C(O)R₅, —OR₅, substituted or unsubstituted phenyl, naphthyl or a heterocyclic group;

R₃ is —H, —NR₄R₅, —N₄C(O)R₅, —OR₅ or a substituted or unsubstituted heterocyclic group, or R₂ and R₃, taken together with the atoms to which they are attached, form an optionally substituted heterocyclic group, or R₃ is absent when Z is O or S;

R₄ and R₅ are independently —H, a substituted or unsubstituted alkyl group, a substituted or unsubstituted aryl group or a substituted or unsubstituted non-aromatic heterocyclic group;

R₆, R₇ and R₈ are independently selected from the group consisting of halogen, —R₄, —CN, —CO₂R₄, —OCOR₄, —C(O)NR₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)NR₄R₅, —NR₄R₅, —NR₄C(O)OR₅, —NR₄C(O)R₅ and —NO₂;

W is C or N;

X is C or N;

Y is C or N;

Z is C, N, O or S, provided that at least two of W, X, Y and Z are C; and

n is 1 or 2.

In a second aspect, the invention provides novel [symbol] -modulating compounds represented by Structural Formula (Ia):

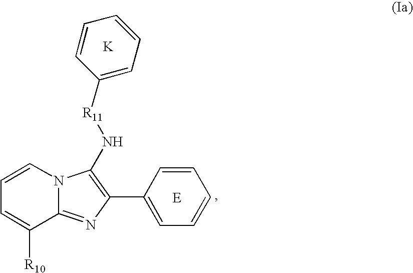

wherein:

R₁₀ is selected from —H, —C(O)—N(R₄₀)(R₅₀), —S(O)₂N(R₄₀)(R₅₀), or —CH—N(R₄₀)(R₅₀); wherein each of R₄₀ and R₅₀ is independently selected from —H, —C₁-C₃ straight or branched alkyl, —(C₁-C₃ straight or branched alkyl)-N(CH₃)₂, —(C₁-C₃ straight or branched alkyl)-heterocyclyl, —(C₁-C₃ straight or branched alkyl)-alkylheterocyclyl, or wherein R₄₀ and R₅₀ taken together with the N atom to which they are bound form a 5-6 membered heterocyclic ring that is optionally substituted with —(C₁-C₃ straight or branched alkyl), and wherein at least one of R₄₀ or R₅₀ is not H;

R₁₁ is selected from —C₁-C₃ straight or branched alkylene or —C(O)—; and

each of ring K and ring E is independently substituted with up to three substituents independently selected from halo, —CF₃, —O—(C₁-C₃ straight or branched alkyl), —S—(C₁-C₃ straight or branched alkyl), —N(R₄₀)(R₅₀), —S(O)—N(R₄₀)(R₅₀), heterocyclyl, (C₁-C₃ straight or branched alkyl)-heterocyclyl, —O—(C₁-C₃ straight or branched alkyl)-heterocyclyl, —S—(C₁-C₃ straight or branched alkyl)-heterocyclyl, or is optionally fused to a 5-6 membered heterocyclyl or heteroaryl, wherein any heterocyclcyl or heteroaryl is optionally substituted with —C₁-C₃ straight or branched alkyl.

In a further aspect, the invention provides novel [symbol] -modulating compounds of Formula (Ib):

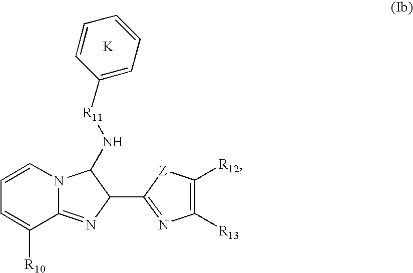

wherein:

Z is selected from O or S;

R₁₀ is selected from —H, C(O)—N(R₄₀)(R₅₀), —S(O)₂N(R₄₀)(R₅₀), or —CH₂—N(R₄₀)(R₅₀); wherein each of R₄₀ and R₅₀ is independently selected from —H, —C₁-C₃ straight or branched alkyl, —(C₁-C₃ straight or branched alkyl)-N(CH₃)₂, —(C₁-C₃ straight or branched alkyl)-heterocyclyl, —(C₁-C₃ straight or branched alkyl)-alkylheterocyclyl, or wherein R₄₀ and R₅₀ taken together with the N atom to which they are bound form a 5-6 membered heterocyclic ring that is optionally substituted with —(C₁-C₃ straight or branched alkyl), and wherein at least one of R₄₀ or R₅₀ is not H.

R₁₁ is selected from —C₁-C₃ straight or branched alkylene or —C(O)—;

each of R₁₂ and R₁₃ is independently selected from —H or —(C₁-C₃ straight or branched alkyl), or R₁₂ and R₁₃ are taken together to form a benzene ring that is substituted with up to two substituents independently selected from —(C₁-C₃ straight or branched alkyl), —CF₃ or halo; and

ring K is substituted with up to three substituents independently selected from halo, —CF₃, —O—(C₁-C₃ straight or branched alkyl), —S—(C₁-C₃ straight or branched alkyl), —N(R₄₀)(R₅₀), —S(O)_{z}—N(R₄₀)(R₅₀), heterocyclyl, (C₁-C₃ straight or branched alkyl)-heterocyclyl, —O—(C₁-C₃ straight or branched alkyl)-heterocyclyl, —S—(C₁-C₃ straight or branched alkyl)-heterocyclyl, or is optionally fused to a 5-6 membered heterocyclyl or heteroaryl, wherein any heterocyclcyl or heteroaryl is optionally substituted with —C₁-C₃ straight or branched alkyl.

In another aspect, the invention provides novel [symbol] -modulating compounds of Formula (II):

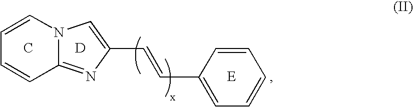

or a salt thereof; where:

Rings C, D and E are optionally substituted; and

x is 0 or 1.

In yet another aspect, the invention provides novel [symbol] -modulating compounds of Formula (VII):

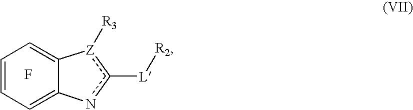

or a salt thereof, where:

Ring F is optionally substituted;

L′ is substituted or unsubstituted phenylene, substituted or unsubstituted thienylene, —C(O)O—, —S—, —CHR₆═CHR₇— or —CHR₈—C(O)—;

R₂ is —H, unsubstituted alkyl, —NR₄R₅, —NR₄C(O)R₅, —OR₅ or a heterocyclic group;

R₃ is —H, —NR₄R₅, —N₄C(O)R₅, —OR₅ or a heterocyclic group, or R₂ and R₃, taken together with the atoms to which they are attached, form an optionally substituted heterocyclic group, or R₃ is absent when Z is O or S;

R₄ and R₅ are independently —H, a substituted or unsubstituted alkyl group, a substituted or unsubstituted aryl group or a substituted or unsubstituted non-aromatic heterocyclic group;

R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)OR₄, —(O), NR₄R₅, —NR₄C(O)OR₅, —NR₄C(O)R₅ and —NO₂;

Z is C, N, O or S; and

n is 1 or 2

In one aspect, the invention provides novel [symbol] modulating compounds of Formula (I):

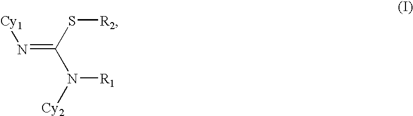

or a salt thereof, where:

Cy₁ and Cy₂ are independently a substituted or unsubstituted cyclic group; and

R₁ and R₂ are independently —H, a substituted or unsubstituted alkyl group, a substituted or unsubstituted aryl group or a substituted or unsubstituted non-aromatic heterocyclic group, or R₁ and R₂ taken together with the atoms to which they are attached from an optionally substituted heterocycle.

In another aspect, the invention provides novel [symbol] -modulating compounds of Formula (II):

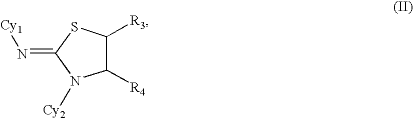

or a salt thereof where:

Cy₁ and Cy₂ are independently a substituted or unsubstituted cyclic group;

R₃ and R₄ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NRC(O)R′, —NO₂, ═O and ═S; and

R and R′ are independently —H, a substituted or unsubstituted alkyl group, a substituted or unsubstituted aryl group or a substituted or unsubstituted non-aromatic heterocyclic group.

The invention also includes salts, prodrugs and metabolites of the compounds disclosed herein.

Also provided are pharmaceutical compositions comprising one or more compounds of Formulas (I)-(II) or a salt, prodrug or metabolite thereof.

In another aspect, the invention provides methods for using [symbol] -modulating compounds, or compostions comprising [symbol] modulating compounds. In certain embodiments, [symbol] -modulating compounds that increase the level and/or activity of a

protein may be used for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, and/or flushing, etc. [symbol] -modulating compounds that increase the level and/or activity of a

protein may also be used for treating a disease or disorder in a subject that would benefit from increased mitochondrial activity, for enhancing muscle performance, for increasing muscle ATP levels, or for treating or preventing muscle tissue damage associated with hypoxia or ischemia. In other embodiments, [symbol] modulating compounds that decrease the level and/or activity of a protein may be used for a variety of therapeutic applications including, for example, increasing cellular sensitivity to stress, increasing apoptosis, treatment of cancer, stimulation of appetite, and/or stimulation of weight gain, etc. As described further below, the methods comprise administering to a subject in need thereof a pharmaceutically effective amount of a

[symbol] -modulating compound.

In certain aspects, the [symbol] -modulating compounds may be administered alone or in combination with other compounds, including other [symbol] -modulating compounds, or other therapeutic agents.

In one aspect, the invention provides novel: [symbol] modulating compounds of Formula (I):

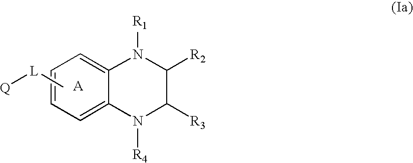

or a salt thereof, wherein:

Ring A optionally has one or more substituents in addition to -L-Q;

L is —C(O)NR—, —NRC(O)—, —NR—NR′—C(O)—, —C(O)—NR—NR′— or —CHR═CHR′—;

Q is a substituted or unsubstituted alkyl group or a substituted or unsubstituted cyclic group, or Q and R taken together with the atoms to which they are attached form a heterocyclic group;

R₁ is —H, a substituted or unsubstituted acyl group, a substituted or unsubstituted alkyl group, a substituted or unsubstituted aryl group or a substituted or unsubstituted non-aromatic heterocyclic group, or R₁ and R₂ taken together with the atoms to which they are attached form an optionally substituted fused ring;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)R, —S(O)ₙOR, —S(O)ₙNRR′, —NRR′, —NRC(O)OR′, —NRC(O)R, —NO₂, ═O and ═S;

R₄ is —H, a substituted or unsubstituted alkyl group, a substituted or unsubstituted alkenyl group, a substituted or unsubstituted aryl group or a substituted or unsubstituted non-aromatic heterocyclic group;

R and R′ are independently —H, a substituted or unsubstituted alkyl group, a substituted or unsubstituted aryl group or a substituted or unsubstituted non-aromatic heterocyclic group; and

n is 1 or 2.

In certain embodiments of the first aspect, novel compounds of the invention are represented by Structural Formula (I):

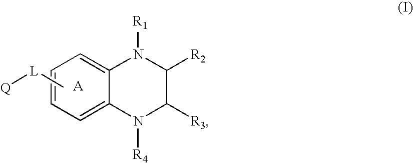

or a salt thereof, wherein:

Ring A optionally has one or more substituents in addition to -L-Cy₁;

L is —C(O)NR—, —NRC(O)—, —NR—NR′—C(O)—, —C(O)—NR—NR′— or —CHR═CHR′—;

Q is a substituted or unsubstituted alkyl group (e.g., an aminoalkyl group) or a substituted or unsubstituted cyclic group;

R₁ is —H, a substituted or unsubstituted acyl group, a substituted or unsubstituted alkyl group, a substituted or unsubstituted aryl group or a substituted or unsubstituted non-aromatic heterocyclic group, or R₁ and R₂ taken together with the atoms to which they are attached form an optionally substituted fused ring;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NRC(O)R′, —NO₂, ═O and ═S;

R₄ is —H, a substituted or unsubstituted alkyl group, a substituted or unsubstituted aryl group or a substituted or unsubstituted non-aromatic heterocyclic group;

R and R′ are independently —H, a substituted or unsubstituted alkyl group, a substituted or unsubstituted aryl group or a substituted or unsubstituted non-aromatic heterocyclic group; and

n is 1 or 2.

In another aspect, the invention provides novel [symbol] -modulating compounds of Formula (II):

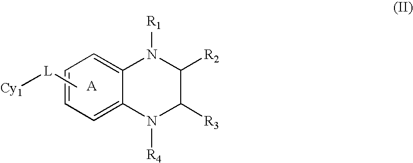

or a salt thereof, wherein:

Ring A optionally has one or more substituents in addition to -L-Cy₁;

Cy₁ is a substituted or unsubstituted cyclic group;

L is —C(O)NR—, —NRC(O)—, —NR—NR′—C(O)—, —C(O)—NR—NR′— or —CHR═CHR′—;

R₁ is —H, a substituted or unsubstituted acyl group, a substituted or unsubstituted alkyl group, a substituted or unsubstituted aryl group or a substituted or unsubstituted non-aromatic heterocyclic group, or R₁ and R₂ taken together with the atoms to which they are attached form an optionally substituted fused ring;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NRC(O)R′, —NO₂, ═O and ═S;

R₄ is —H, a substituted or unsubstituted alkyl group, a substituted or unsubstituted aryl group or a substituted or unsubstituted non-aromatic heterocyclic group;

R and R′ are independently —H, a substituted or unsubstituted alkyl group, a substituted or unsubstituted aryl group or a substituted or unsubstituted non-aromatic heterocyclic group; and

n is 1 or 2.

In yet another aspect, the invention provides novel [symbol] -modulating compounds of Formula (III):

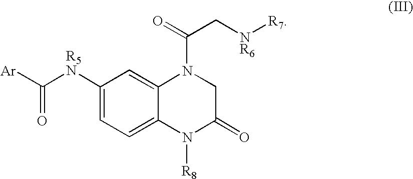

or a salt thereof, wherein:

Ar is a substituted or unsubstituted aryl group;

R₅, R₆, R₇ and R₈ are independently —H, a substituted or unsubstituted alkyl group, a substituted or unsubstituted aryl group or a substituted or unsubstituted non-aromatic heterocyclic group.

The invention also includes salts, prodrugs and metabolites of the compounds disclosed herein.

Also provided are pharmaceutical compositions comprising one or more compounds disclosed herein, including salts, prodrugs and metabolites thereof.

In one aspect, the invention provides novel [symbol] -modulating compounds of Formula (Ia):

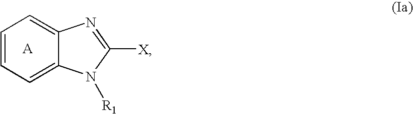

or a salt thereof, wherein:

Ring A is optionally substituted;

R₁ is —H, a substituted or unsubstituted alkyl group, a substituted or unsubstituted alkenyl group, a substituted or unsubstituted aryl group or a substituted or unsubstituted heterocyclic group;

X is —(C—R₂)ₘ—NHCO—R₃, —(CHR₂)—NHCONR₄R₅, —(CH₂)—NR₁SO₂—R₃, —SR₃ or -arylene-R₂;

each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₃ is a substituted or unsubstituted alkyl group, a substituted or unsubstituted aryl group or a substituted or unsubstituted heterocyclic group;

R₄ and R₅ are independently —H, a substituted or unsubstituted alkyl group, a substituted or unsubstituted aryl group or a substituted or unsubstituted non-aromatic heterocyclic group;

m is an integer from 2 to 12;

n is 1 or 2; and

r is an integer from 0 to 12.

In another aspect, the invention provides novel [symbol] modulating compounds of Formula (I):

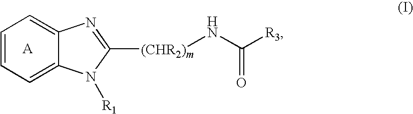

or a salt thereof, where:

Ring A is optionally substituted;

R₁ is —H, a substituted or unsubstituted alkyl group, a substituted or unsubstituted alkenyl group, a substituted or unsubstituted aryl group or a substituted or unsubstituted heterocyclic group;

each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₃ is a substituted or unsubstituted alkyl group, a substituted or unsubstituted aryl group or a substituted or unsubstituted heterocyclic group;

R₄ and R₅ are independently —H, a substituted or unsubstituted alkyl group, a substituted or unsubstituted aryl group or a substituted or unsubstituted heterocyclic group;

m is an integer from 2 to 12; and

n is 1 or 2.

In yet another aspect, the invention provides novel [symbol] -modulating compounds of Formula (II):

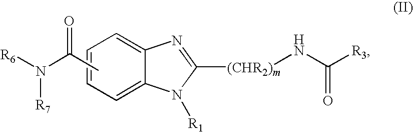

or a salt thereof, where:

R₁ is —H, a substituted or unsubstituted alkyl group, a substituted or unsubstituted aryl group or a substituted or unsubstituted heterocyclic group;

each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)OR₄, —S(O)ₙNR₄R₅, —NR₄R₅, —NR₄C(O)OR₅, —NR₄C(O)R₅ and —NO₂;

R₃ is a substituted or unsubstituted alkyl group, a substituted or unsubstituted aryl group or a substituted or unsubstituted heterocyclic group;

R₄ and R₅ are independently —H, a substituted or unsubstituted alkyl group, a substituted or unsubstituted aryl group or a substituted or unsubstituted heterocyclic group;

R₆ and R₇ are independently —I, a substituted or unsubstituted alkyl group, a substituted or unsubstituted aryl group or a substituted or unsubstituted heterocyclic group, or R₆ and R₇ taken together with the nitrogen atom to which they are attached form a heterocyclic ring;

m is an integer from 2 to 12; and

n is 1 or 2.

The invention also includes salts, prodrugs and metabolites of the compounds disclosed herein.

Also provided are pharmaceutical compositions comprising one or more compounds disclosed herein, or a salt, prodrug or metabolite thereof.

In one aspect, the invention provides novel activating compounds of Formula (Ia):

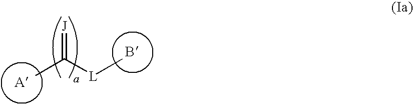

or a salt thereof, wherein:
Ring A′ is a 5- to 7-membered ring optionally fused to a second 5- to 7-membered ring, which is optionally substituted with one to three functional groups selected from the group consisting of halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR, —NRC(O)R, —NO₂, substituted or unsubstituted alkyl, substituted or unsubstituted alkenyl, substituted or unsubstituted non-aromatic heterocyclic and substituted or unsubstituted aryl;
Ring B′ is a 5- to 7-membered ring optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR, —NRC(O)R, —NO₂, substituted or unsubstituted alkyl, substituted or unsubstituted alkenyl, substituted or unsubstituted non-aromatic heterocyclic and substituted or unsubstituted aryl;
J is O or S;
L is —C═C— or —NH—(CH₂)ₖ—;
R and R′ are independently —H, a substituted or unsubstituted alkyl group, a substituted or unsubstituted alkenyl group, a substituted or unsubstituted non-aromatic heterocyclic group or a substituted or unsubstituted aryl group;
a is 0 or 1;
k is an integer from 1 to 4; and
n is 1 or 2,

provided that Ring A′ and Ring B′ are not both phenyl and at least one is substituted with at least one hydrogen bond donating group, and provided that the compound is not 4-((E)-2-(pyridin-4-yl)vinyl)phenol.

In another aspect, the invention provides novel activating compounds of Formula (I):

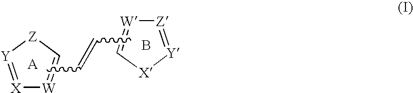

or a salt thereof, wherein:
W is CH or N;
X is CH or N;
Y is CH or N;
Z is S, O or NH;
W′ is CH or N;
X′ is CH or N;
Y′ is CH or N;
Z′ is S, O or NH;

R and R′ are independently —H, a substituted or unsubstituted alkyl group, a substituted or unsubstituted alkenyl group, a substituted or unsubstituted non-aromatic heterocyclic group or a substituted or unsubstituted aryl group;

n is 1 or 2;

Ring A is substituted with at least one hydrogen bond donating group and is optionally substituted with one to three functional groups selected from the group consisting of halogen, —OR, —CN, —CO₂R, —OCOR, —OCO₂R, —C(O)NRR′, —OC(O)NRR′, —C(O)R, —COR, —SR, —OSO₃H, —S(O)R, —S(O)ₙOR, —S(O)NRR′, —NRR′, —NRC(O)OR, —NRC(O)R, —NO₂, substituted or unsubstituted alkyl, substituted or unsubstituted alkenyl, substituted or unsubstituted non-aromatic heterocyclic and substituted or unsubstituted aryl; and

Ring B is optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)R, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.

In another aspect, the invention provides novel: compounds of Formulas (I)-(XVIII) (including (IA)), including salts, prodrugs and metabolites thereof. Also provided are pharmaceutical compositions comprising a compounds of Formulas (I)-(XVIII) (including (IA)), or a salt prodrug or metabolites thereof.

In one embodiment, the invention is a method for promoting survival of a eukaryotic cell comprising contacting the cell with at least one compound of Structural Formula (I) or (II):

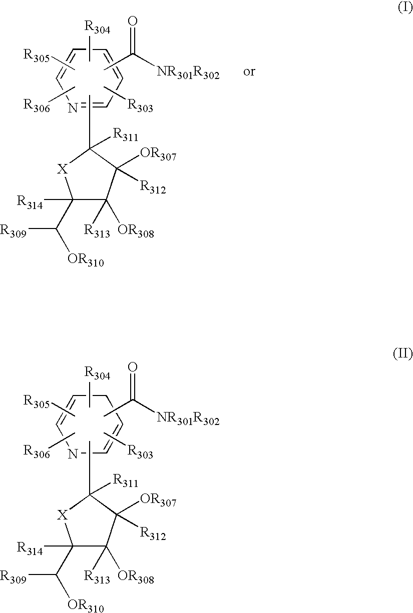

or pharmaceutically acceptable salt thereof where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)R, —S(O)OR, —S(O)ₙNRR′, —NRR′, —NRC(O)OR′ and —NRC(O)R;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)OR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, the invention is method for treating or preventing a disease or disorder associated with cell death, cell dysfunction or aging in a subject, comprising administering to a subject in need thereof a therapeutically effective amount of at least one compound of Structural Formula (I) or (II):

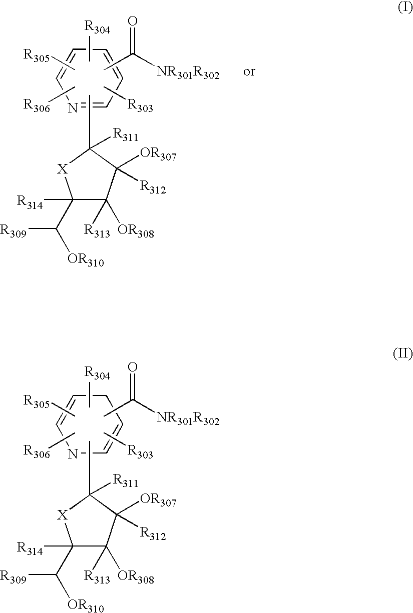

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In yet another embodiment, the invention is a method for treating or preventing insulin resistance, a metabolic syndrome, hypercholesterolemia, artherogenic dyslipidemia, diabetes, or complications thereof, or for increasing insulin sensitivity in a subject, comprising administering to a subject in need thereof a therapeutically effective amount of at least one compound of Structural Formula (I) or (II):

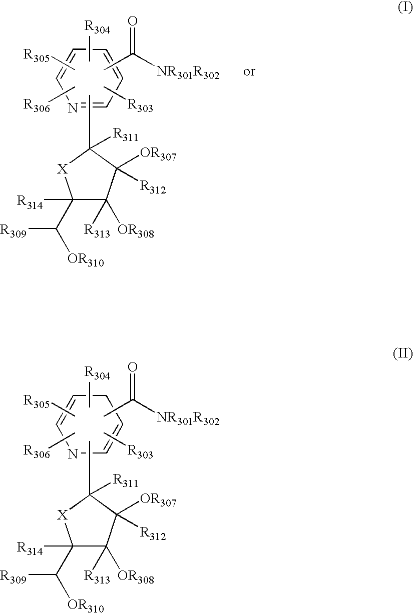

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)R, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2,

provided that when X is O and R₃₀₁-R₃₀₉ and R₃₁₁-R₃₁₄ are —H, R₃₁₀ is not —H.

In a further embodiment, the invention is method for reducing the weight of a subject, or preventing weight gain in a subject, comprising administering to a subject in need thereof a therapeutically effective amount of at least one compound of Structural Formula (I) or (II):

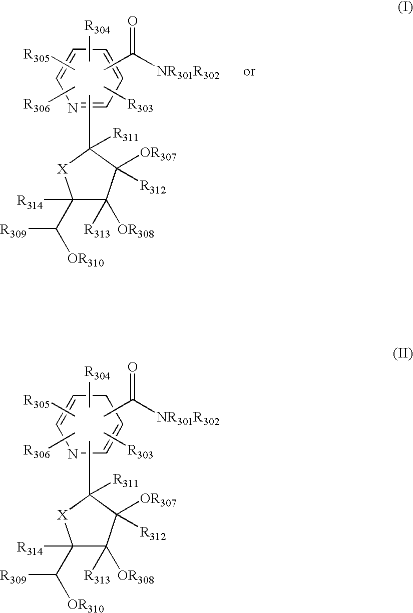

or a pharmaceutically acceptable salt thereof; wherein:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)OR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently-H, a substituted or unsubstituted alkyl group, a substituted or unsubstituted aryl group or a substituted or unsubstituted non-aromatic heterocyclic group;

X is O or S; and

n is 1 or 2.

In one embodiment, the invention is a method for preventing the differentiation of a pre-adipocyte, comprising contacting the pre-adipocyte with at least one compound of Structural Formula (I) or (II):

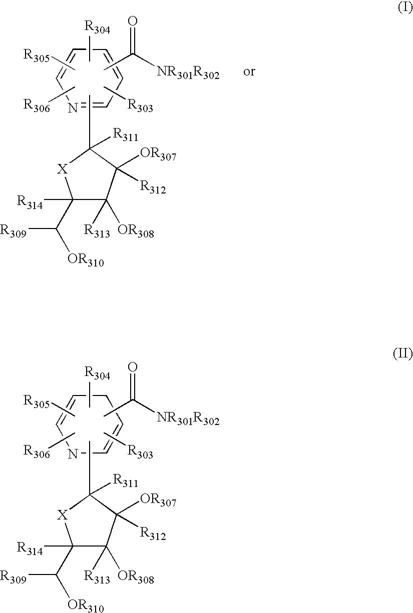

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with, the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)NRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR, —NO₂ and —NRC(O)R;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, the invention is a method for prolonging the lifespan of a subject comprising administering to a subject a therapeutically effective amount of at least one compound of Structural Formula (I) or (II):

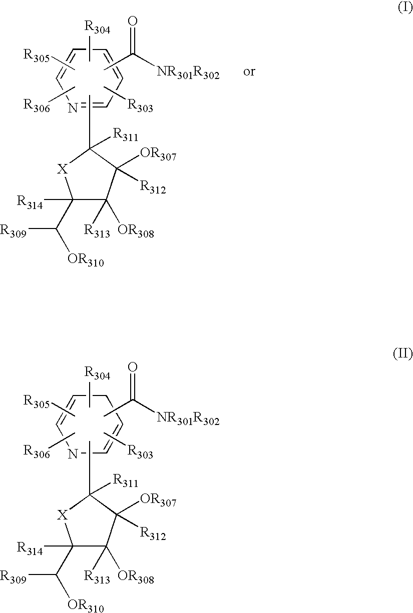

or a pharmaceutically acceptable salt thereof; where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)OR, —S(O)ₙNRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)R, —S(O)OR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In yet another embodiment, the invention is a method for treating or preventing a neurodegenerative disorder in a subject, comprising administering to a subject in need thereof a therapeutically effective amount of at least one compound of Structural Formula (I) or (II):

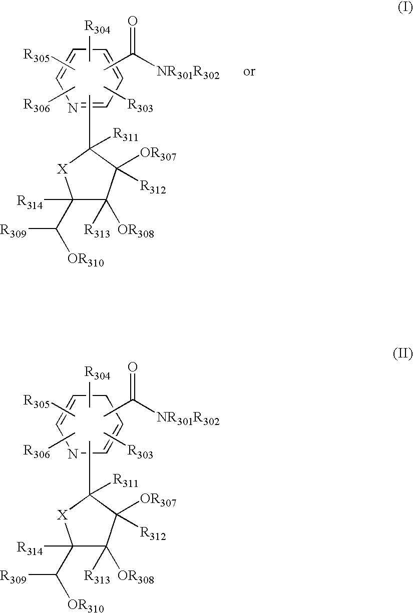

or a pharmaceutically acceptable salt thereof where:

R₃₀₁ and R₃₀₂ are independently-H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)NRR′, —NRR′, —NRC(O)OR, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(Oh)R, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In a further embodiment, the invention is a method of for treating or preventing a blood coagulation disorder in a subject, comprising administering to a subject in need thereof a therapeutically effective amount of at least one compound of Structural Formula (I) or (II):

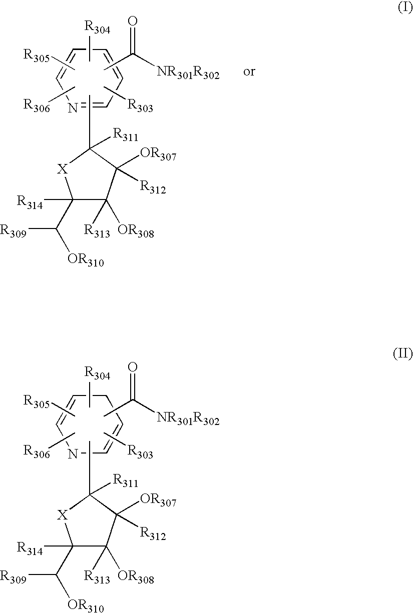

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)NRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)₁′OR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In the above methods, the compound represented by Structural Formula (I) or (II) is typically represented by Structural Formula (III) or (IV):

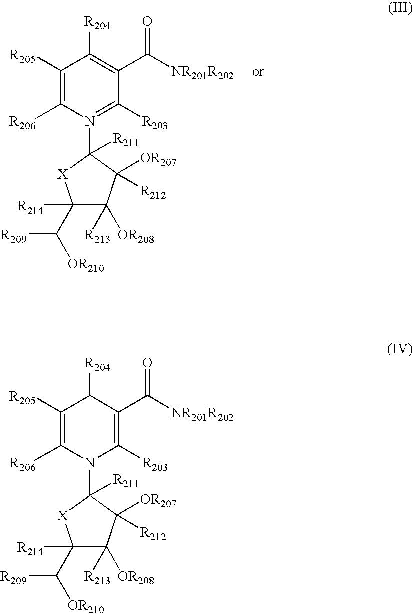

or a pharmaceutically acceptable salt thereof, where:

R₂₀₁ and R₂₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₂₀₁ and R₂₀₂ taken together with the atom to which they are attached form a substituted or unsubstituted non-aromatic heterocyclic group;

R₂₀₃, R₂₀₄, R₂₀₅ and R₂₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO_{2l R, —OCOR, —OCO}₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₂₀₇, R₂₀₈ and R₂₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₂₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₂₁₁, R₂₁₂, R₂₁₃ and R₂₁₄ are independently selected from the group consisting of —H, a substituted or unsubstituted alkyl group, a substituted or unsubstituted aryl group, a substituted, or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

Another aspect of the invention is a pharmaceutical composition comprising a pharmaceutically acceptable carrier or diluent and a compound represented by Structural Formula (V) or (VI):

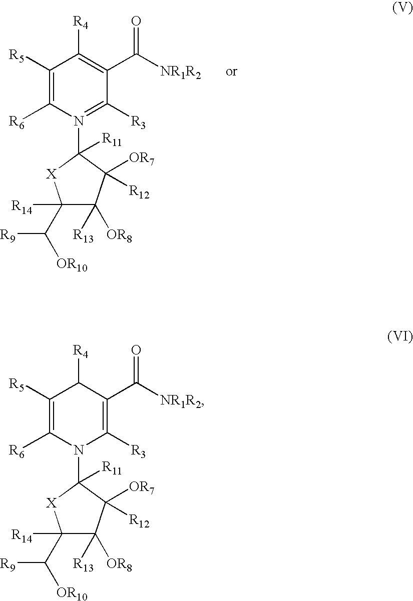

or a pharmaceutically acceptable salt thereof, where:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together with the atom to which they are attached form a substituted or unsubstituted non-aromatic heterocyclic group, provided that when one of R₁ and R₂ is —H, the other is not an alkyl group substituted by —C(O)OCH₂ CH₃;

R₃, R₄ and R₅ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₆ is selected from the group consisting of —H, a substituted or unsubstituted alkyl group; a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRC(O)OR′, —NO₂ and —NRC(O)R′;

R₇, R₈ and R₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)₈ OR, —S(O)ₙNRR′, —NRR′, —NRC(O)OR′ and —NRC(O)R′;

R₁₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2,

provided that R₁-R₁₄ are not each —H and that R₁-R₉ and R₁-R₁₄ are not each —H when R₁₀ is —C(O)C₆H₅.

In one embodiment, the invention is a pharmaceutical composition comprising a pharmaceutically acceptable carrier or diluent and a compound represented by Structural Formula (VII) or (VIII):

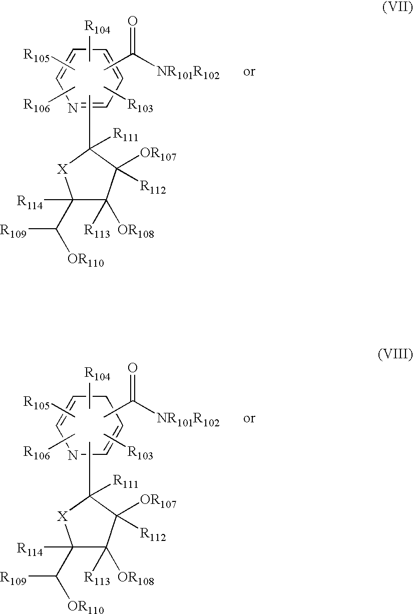

or a pharmaceutically acceptable salt thereof wherein:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₁₀ and R₁₀₂ taken together with the atom to which they are attached form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)OR, —S(O)NRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR,

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)R, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR; —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′.

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, the invention is a pharmaceutical composition comprising a pharmaceutically acceptable carrier or diluent and a compound represented by Structural Formula (IX) or (X):

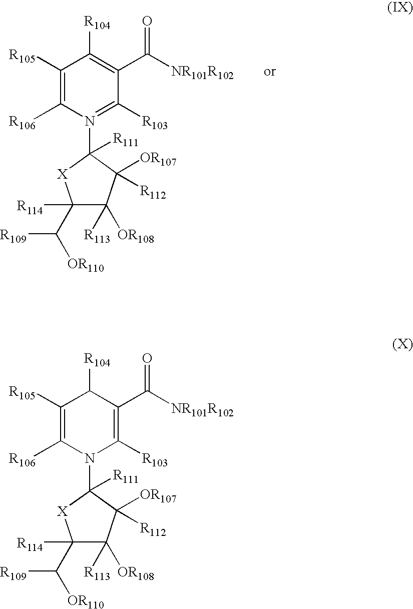

or unsubstituted aryl group, or R₁₁₀ and R₁₂ taken together with the atom to which they are attached form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)R, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NR, —C(S)R, —C(S)OR or —C(O)SR,

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)NRR′, —NRR′, —NRC(O)OR′, —NO₂ and —NRC(O)R′;

R and R′ are independently-H, a substituted or unsubstituted alkyl group, a substituted or unsubstituted aryl group or a substituted or unsubstituted non-aromatic heterocyclic group;

X is O or S; and

n is 1 or 2.

A further aspect of the invention is a compound represented by Structural Formula (XI) or (XII):

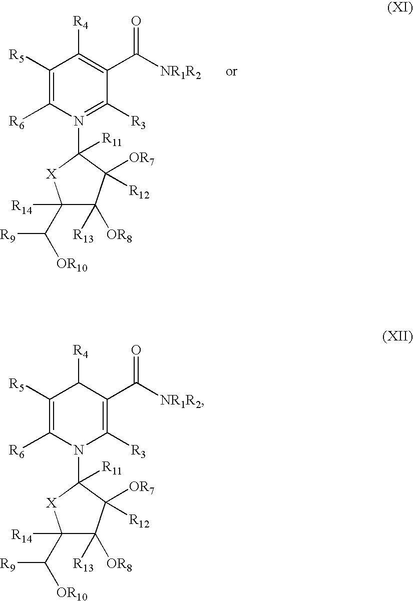

or a pharmaceutically acceptable salt thereof, where:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together with the atom to which they are attached form a substituted or unsubstituted non-aromatic heterocyclic group, provided that when one of R₁ and R₂ is —H, the other is not a 2,2,6,6-tetramethyl-1-oxypiperidin-4-yl group and is not an alkyl group substituted by —C(O)OCH₂CH₃;

R₃ and R₄ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)ₙOR′, —NO₂ and —NRC(O)R′;

R₅ is selected from the group consisting of —H, a substituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₆ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRC(O)OR′, —NO₂ and —NRC(O)R′;

R₇, R₈ and R₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁, R₄₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2,

provided that R₁-R₆ are not each —H.

Yet another aspect of the invention is a compound represented by Structural Formula (XI) or (XII):

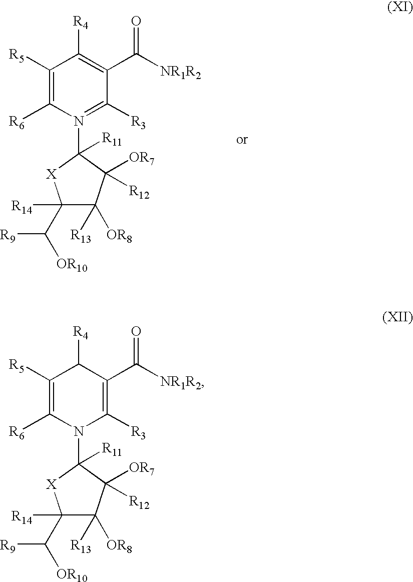

or a pharmaceutically acceptable salt thereof, wherein:

R₁ and R₂ are independently-H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together with the atom to which they are attached form a substituted or unsubstituted non-aromatic heterocyclic group;

R₃, R₄, R₅ and R₆ are independently selected from the group consisting of —H, a substituted or unsubstituted alkyl group, a substituted or unsubstituted aryl group, a substituted or unsubstituted non-aromatic heterocyclic group; halogen, —OR, —CN, —CO₂R, —OCOR; —OCO₂R, —C(O)NRR′, —OC(O)NRR′, —C(O)R, —COR, —SR, —OSO₃H, —S(O)ₙR, —S(O)ₙOR, —S(O)NRR′, —NRR′, —NRC(O)OR′, —NO₂ and —NRC(O)R′;

R₇, R₈ and R₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₇ and R₈ is a substituted or unsubstituted alkyl group, a substituted or unsubstituted aryl group, —C(O)R, —C(O)OR, —C(O)NHR, —C(S)R, —C(S)OR or —C(O)SR, provided that none of R₇ and R₈ are —C(O)C₆H₅ and that R₇ and R₈ are not both —C(O)CH₃ or —C(O)C₆H₄F;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)NRR′, —NRR′, —NRC(O)OR′ and —NRC(O)R′;

R₁₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

A further aspect of the invention is a compound represented by Structural Formula (XI) or (XII):

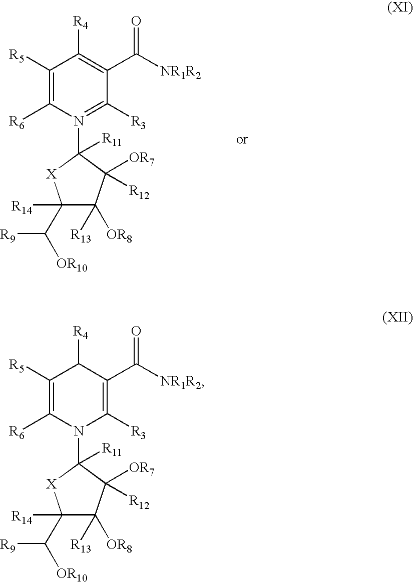

or a pharmaceutically acceptable salt thereof, where:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together with the atom to which they are attached form a substituted or unsubstituted non-aromatic heterocyclic group;

R₃, R₄, R₅ and R₆ are independently selected from the group consisting of H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₇ and R⁸ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₀ is selected from the group consisting of a substituted or unsubstituted alkyl group, a substituted or unsubstituted aryl group, —C(O)R, —C(O)OR, —C(O)NHR, —C(S)R, —C(S)OR and —C(O)SR, provided that R₁₀ is not —C(C₆H₅)₃, —C(O)C₆H₅, —C(O)CH₃, —C(O)C₆H₄F or —C(O)CH(OC(O)CH₃)CH(CH₃)CH₂ CH₃;

R₁₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In one embodiment, sirtuin-modulating compounds of the invention are represented by Structural Formula I:

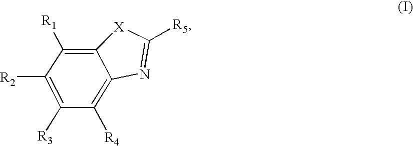

wherein:

one of R₁, R₂, R₃ or R₄ is selected from a 5- to 6-membered heteroaryl comprising 1 to 3 heteroatoms independently selected from N, O or S, wherein said heteroaryl is optionally benzofused or optionally fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

the others of R₁, R₂, R₃ and R₄ are independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, when X is NR₆, R₆ is hydrogen, and R₂ or R₃ is 1,2,4-oxadiazole-3-yl, pyridin-4-yl, or furan-2-yl, then R₅ is not phenyl substituted with an acylamino group.

In certain embodiments, when X is NR₆, R₆ is H or alkoxymethyl, and R₂ or R₃ is substituted or unsubstituted 1H-imidazo[4,5-b]pyridin-2-yl, substituted or unsubstituted 1H-benzimidazol-2-yl, or substituted or unsubstituted oxazolo[5,4-b]pyridin-2-yl, then R₅ is not phenyl monosubstituted with hydroxy or methoxy.

In certain embodiments, when X is NR₆ s R₆ is H or alkoxymethyl, and R₂ or R₃ is benzo[b]thienyl, then R₅ is not 2H-indazol-3-yl.

In certain embodiments, when X is NR₆, R₆ is H or alkoxymethyl and R₂ or R₃ is 1H-imidazo[2,1-b]thiazol-3-yl, then R₅ is not unsubstituted pyridin-4-yl.

In certain embodiments, when X is NR₆ and R₂ or R₃ is 1,2,4-oxadiazol-3-yl, then R₅ is not 2-chloro-5-nitrophenyl.

In certain embodiments, when X is NR₆ and R₂ or R₃ is thien-3-yl or furan-2-yl, then R₅ is not unsubstituted 2H-indazol-3-yl.

In certain embodiments, when X is NR₆ and R₂ or R₃ is imidazol-4-yl or thiazolyl, then R₅ is not unsubstituted thiazol-4-yl.

In certain embodiments, when X is NR₆ and R₂ or R₃ is pyridazin-3-yl, then R₅ is not 4-methylphenyl or 4-methoxyphenyl.

In certain embodiments, when X is NR₆ and R₂ or R₃ is pyridinyl, then R₅ is not unsubstituted phenyl, 4-fluorophenyl, 4-methylphenyl, 4-(1,1-dimethylethyl)phenyl, or 4-trifluoromethylphenyl.

In certain embodiments, when X is S and R₂ or R₃ is substituted or unsubstituted benzothiazol-2-yl, then R₅ is not unsubstituted phenyl, 2-methyl substituted phenyl, 4-nitrophenyl, 4-amino substituted phenyl, 3- or 4-methylaminophenyl, 4-thiophenyl, 4-hydroxyphenyl, 4-(sulfonatomethyl)aminophenyl, 2-methyl substituted benzthiazol-6-yl, 2,3-dimethylbenzthiazol-6-yl, 4-amino-3-sulfonatophenyl, 4-amino-3-iodophenyl, 4-(4-acetamidophenylsulfamoyl)phenyl, or 4-(4-dimethylaminophenyl)iminophenyl.

In certain embodiments, when X is S and R₂ is substituted or unsubstituted pyrimidinyl, then R₅ is not phenyl substituted with an octyloxy-, propoxypropoxy-, glycine- or hexanoic acid-containing substituent and optionally one or more additional substituents.

In certain embodiments, when X is O and R₄ is substituted or unsubstituted benzoxazol-2-yl, then R₅ is not phenyl, 2-hydroxyphenyl, 2-methoxyphenyl, 2-phenylmethoxyphenyl, 2-hydroxy-3-phenylmethylphenyl, or 2-hydroxy-6-methylphenyl.

In certain embodiments, when X is O and R₄ is substituted or unsubstituted 1H-benzimidazol-2-yl, then R₅ is not 2-hydroxyphenyl.

In certain embodiments, when X is O and one of R₂ or R₃ is substituted or unsubstituted 1H-benzimidazol-2-yl, substituted or unsubstituted oxazolo[5,4-b]pyridin-2-yl or substituted or unsubstituted 1H-imidazo[4,5-b]pyridin-2-yl, then R₅ is not 4-hydroxyphenyl, 4-methoxyphenyl, 4-N,N-bis-(2-chloroethyl)aminophenyl, or naphthalen-4-yl.

In certain embodiments, when X is O and R₃ is 1-methyl-1H-pyrrol-2-yl, then R₅ is not 3-chloro-4-(5-methoxyfuran-2-yl)ethenylphenyl or 3-chloro-4-(5-methoxythien-2-yl)ethenylphenyl.

In certain embodiments, when X is O and R₂ is benzo[b]thien-2-yl, then R₅ is not 2-methoxy-5-aminophenyl, 3-amino-4-methoxyphenyl, 2-propylamino-5-aminophenyl, 2-methoxy-5-nitrophenyl, 4-methoxy-3-nitrophenyl, or 5-nitro-2-propylaminophenyl.

In certain embodiments, when X is O and one of R₂ or R₃ is benzofuran-2-y, then R₅ is not 2-methoxy-5-aminophenyl, 2-propylamino-5-aminophenyl, 2-fluoro-5-nitrophenyl, 2-methoxy-5-nitrophenyl, 4-methoxy-3-nitrophenyl, or 5-nitro-2-propylaminophenyl.

In certain embodiments, when X is O and one of R₂ or R₃ is substituted or unsubstituted benzoxazol-2-yl, then R₅ is not 4-hydroxyphenyl, 4-styrylphenyl, 4-tert-butylphenyl, or 5-phenylthien-2-yl.

In certain embodiments, when X is O and R₁ is substituted or unsubstituted benzoxazol-2-yl, then R₅ is not 2-methoxyphenyl.

In certain embodiments, when X is O and one of R₂, R₃ or R₄ is furan-2-yl, then R₅ is not substituted or unsubstituted phenyl, benzo[b]thien-2-yl, benzoxazol-2-yl, naphthalen-2-yl, benzofuran-2-yl, quinolin-6-yl, or 5-methylthien-2-yl.

In certain embodiments, when X is O and R₃ is pyridin-3-yl, then R₅ is not 3-methoxyphenyl.

In certain embodiments, when X is O and one of R₂ or R₃ is substituted or unsubstituted pyrimidin-5-yl, then R₅ is not phenyl.

In certain embodiments, when X is O and R₃ is thiazol-2-yl, then R₅ is not thiazol-4-yl.

In certain embodiments, when X is O and one of R₂ or R₃ is substituted or unsubstituted thien-2-yl, then R₅ is not 3-amino-4-methoxyphenyl, 2-hydroxyphenyl, 3-[5-carboxylic acid-2,3-dihydro-1,3-dioxo-1H-isoindol-1-yl]-4-methoxyphenyl or 3-nitro-4-methoxyphenyl.

In another embodiment, sirtuin-modulating compounds of the invention are represented by Structural Formula II:

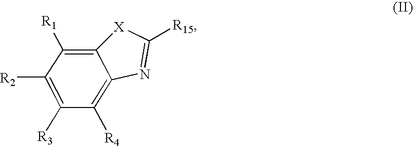

wherein:

one of R₂ or R₃ is selected from a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₁, R₄, and the other of R₂ or R₃ are each independently selected from hydrogen, a solubilizing group, or a C₁-C₄ straight or branched alkyl, wherein at least two of R₁, R₂, R₃ and R₄ are hydrogen;

X is selected from O, S or NR₆, wherein R₆ is selected from hydrogen, a solubilizing group, or a C₁-C₄ straight or branched alkyl; and

R₁₅ is an optionally substituted heteroaryl or an optionally substituted naphthalenyl.

In certain embodiments, when X is NR₆, R₆ is hydrogen, and R₂ or R₃ is 1,2,4-oxadiazole-3-yl, pyridin-4-yl, or furan-2-yl, then R₅ is not phenyl substituted with an acylamino group.

In certain embodiments, when X is NR₆ and R₂ or R₃ is benzo[b]thienyl, then R₅ is not 2H-indazol-3-yl.

In certain embodiments, when X is NR₆ and R₂ or R₃ is 1H-imidazo[2,1-b]thiazol-3-yl, then R₅ is not unsubstituted pyridin-4-yl.

In certain embodiments, when X is NR₆ and R₂ or R₃ is thien-3-yl or furan-2-yl, then R₅ is not unsubstituted 2H-indazol-3-yl.

In certain embodiments, when X is NR₆ and R₂ or R₃ is imidazol-4-yl or thiazolyl, then R₅ is not unsubstituted thiazol-4-yl.

In certain embodiments, when X is S and R₂ is benzothiazol-2-yl, then R₅ is not 2-methyl substituted benzthiazol-6-yl.

In certain embodiments, when X is O and one of R₂ or R₃ is benzoxazol-2-yl, then R₅ is not 5-phenylthien-2-yl.

In certain embodiments, when X is O and R₃ is thiazol-2-yl, then R₅ is not thiazol-4-yl.

In certain embodiments, when X is O and one of R₂ or R₃ is furan-2-yl, then R₅ is not unsubstituted benzo[b]thien-2-yl, unsubstituted benzoxazol-2-yl, or unsubstituted naphthalen-2-yl.

In another embodiment, sirtuin-modulating compounds of the invention are represented by Structural Formula III:

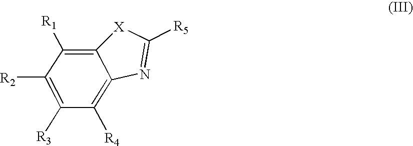

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, when X is O and R₄ is benzoxazol-2-yl, then R₅ is not unsubstituted phenyl or, 2-hydroxy, 2-methoxy or 2-phenylmethoxy substituted phenyl.

In certain embodiments, when X is O and R₄ is benzimidazole-2-yl, then R₅ is not 2-hydroxyphenyl.

In certain embodiments, when X is O and R₁ is benzoxazol-2-yl, then R₅ is not 2-methoxyphenyl.

In certain embodiments, one of R₁-R₄ is selected from:

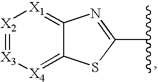

or

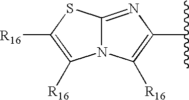

wherein:

X₁, X₂, X₃, and X₄ are each independently selected from CR₁₆ or N; and

two or three occurrences of R₁₆ are H and the other(s) is a substituent selected from a solubilizing group, or a C₁-C₄ straight or branched alkyl.

In certain embodiments, R₅ is selected from phenyl, 1H-pyrrolyl, pyridinyl, quinolinyl, quinoxalinyl, thienyl, or furanyl, wherein R₅ is optionally substituted.

In certain embodiments, R₅ is selected from 3,4-dimethoxyphenyl, 4-dimethylaminophenyl, 4-methoxyphenyl, 4-morpholinophenyl, or 3,4-dioxymethylenephenyl.

In another embodiment, sirtuin-modulating compounds of the invention are represented by Structural Formula IIIa:

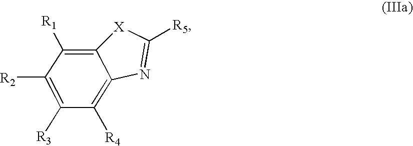

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, one of R₁-R₄ is selected from:

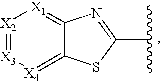

or

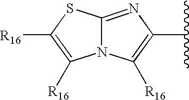

wherein:

X₁, X₂, X₃, and X₄ are each independently selected from CR₁₆ or N; and

two or three occurrences of R₁₆ are H and the other(s) is a substituent selected from a solubilizing group, or a C₁-C₄ straight or branched alkyl.

In certain embodiments, R₅ is selected from phenyl, 1H-pyrrolyl, pyridinyl, quinolinyl, quinoxalinyl, thienyl, or furanyl, wherein R₅ is optionally substituted.

In certain embodiments, R₅ is selected from 3,4-dimethoxyphenyl, 4-dimethylaminophenyl, 4-methoxyphenyl, 4-morpholinophenyl, or 3,4-dioxymethylenephenyl.

In another embodiment, sirtuin-modulating compounds of the invention are represented by Structural Formula IIIb:

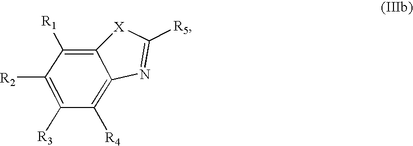

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein when said heteroaryl is benzofused, said heteroaryl comprises a S atom, and wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, one of R₁-R₄ is selected from:

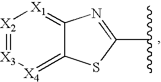

or

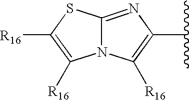

wherein:

X₁, X₂, X₃, and X₄ are each independently selected from CR₁₆ or N; and

two or three occurrences of R₁₆ are H and the other(s) is a substituent selected from a solubilizing group, or a C₁-C₄ straight or branched alkyl.

In certain embodiments, R₅ is selected from phenyl, 1H-pyrrolyl, pyridinyl, quinolinyl, quinoxalinyl, thienyl, or furanyl, wherein R₅ is optionally substituted.

In certain embodiments, R₅ is selected from 3,4-dimethoxyphenyl, 4-dimethylaminophenyl, 4-methoxyphenyl, 4-morpholinophenyl, or 3,4-dioxymethylenephenyl.

In another embodiment, sirtuin-modulating compounds of the invention are represented by Structural Formula IV:

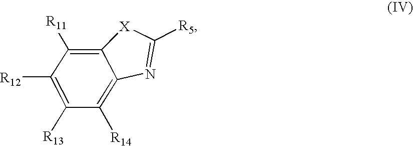

wherein:

one of R₁₁-R₁₄ is an 8- to 10-membered bicyclic heteroaryl comprising at least one heteroatom selected from N, O or S in each ring, wherein at least one of said heteroatoms in the bicyclic heteroaryl is an S atom, wherein said heteroaryl is bound to the rest of the compound via a carbon ring atom, and wherein said heteroaryl is optionally substituted on a single carbon ring atom with a substituent selected from a solubilizing group, or a C₁-C₄ straight or branched alkyl;

and the remainder of R₁₁-R₁₄ are each independently selected from hydrogen, a solubilizing group, or a C₁-C₄ straight or branched alkyl, wherein at least two of R₁₁, R₁₂, R₁₃ or R₁₄ are hydrogen;

X is selected from O, S or NR₆, wherein R₆ is selected from hydrogen, a solubilizing group, or a C₁-C₄ straight or branched alkyl; and

R₅ is an optionally substituted aryl or heteroaryl.

In certain embodiments, when R₅ is 4-pyridyl, R₅ is a substituted 4-pyridyl. In certain such embodiments, the 4-pyridyl may be substituted with halogen, hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl. In certain embodiments, the 4-pyridyl may be substituted with hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl.

In certain embodiments, X is NR₆.

In another embodiment, sirtuin-modulating compounds of the invention are represented by Structural Formula IVa:

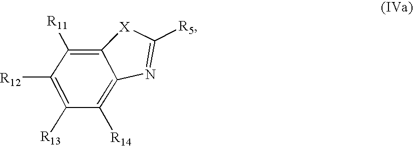

wherein:

one of R₁₂ or R₁₃ is an 8- to 10-membered bicyclic heteroaryl comprising at least one heteroatom selected from N, O or S in each ring, wherein at least one of said heteroatoms in the bicyclic heteroaryl is an S atom, wherein said heteroaryl is bound to the rest of the compound via a carbon ring atom, and wherein said heteroaryl is optionally substituted on a single carbon ring atom with a substituent selected from a solubilizing group, or a C₁-C₄ straight or branched alkyl;

R₁₁, R₁₄ and the other of R₁₂ or R₁₃ are each independently selected from hydrogen, a solubilizing group, or a C₁-C₄ straight or branched alkyl, wherein at least two of R₁₁, R₁₂, R₁₃ or R₁₄ are hydrogen;

X is selected from O, S or NR₆, wherein R₆ is selected from hydrogen, a solubilizing group, or a C₁-C₄ straight or branched alkyl; and

R₅ is an optionally substituted phenyl.

In certain embodiments, X is NR₆.

In another embodiment, sirtuin-modulating compounds of the invention are represented by Structural Formula V:

wherein:

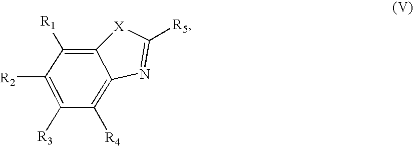

one of R₂, or R₃ or R₄ is selected from

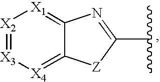

or

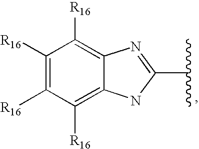

wherein:
one of X₁, X₂, X₃ or X₄ is N and the others are each CR₁₆;
Z is selected from NH, or O; and
each R₁₆ is independently selected from hydrogen, a solubilizing group, or a C₁-C₄ straight or branched alkyl, wherein no more than one R₁₆ is selected from a solubilizing group, or a C₁-C₄ straight or branched alkyl;

R₁, R₄ and the other of R₂ and R₃ are each independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted phenyl.

In certain embodiments, when X is NR₆ and R₂ or R₃ is 1H-imidazo[4,5-b]pyridin-2-yl, 1H-benzimidazol-2-yl, or oxazolo[5,4-b]pyridin-2-yl, and R₅ is phenyl substituted with hydroxy or methoxy, then there is at least one additional substituent. In certain such embodiments, the at least one additional substituent is selected from halogen, hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl. In certain embodiments, the at least one additional substituent is selected from hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl.

In certain embodiments, when X is O and R₄ is 1H-benzimidazole-2-yl, then R₅ is not 2-hydroxyphenyl.

In certain embodiments, when X is O and one of R₂ or R₃ is 1H-benzimidazol-2-yl, oxazolo[5,4-b]pyridin-2-yl or 1H-imidazo[4,5-b]pyridin-2-yl, then R₅ is not 4-hydroxyphenyl, 4-methoxyphenyl, or 4-N,N-bis(2-chloroethyl)aminophenyl.

In another embodiment, sirtuin-modulating compounds of the invention are represented by Structural Formula VI:

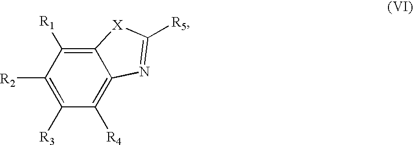

wherein:

one of R₂ or R₃ is selected from

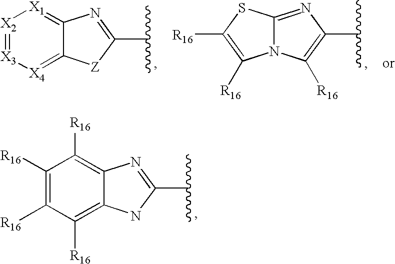

wherein:
one of X₁, X₂, X₃ or X₄ and the others are each CR₁₆;
Z is selected from NH, S or O; and
each R₁₆ is independently selected from hydrogen, a solubilizing group, or a C₁-C₄ straight or branched alkyl, wherein no more than one R₁₆ is selected from a solubilizing group, or a C₁-C₄ straight or branched alkyl;

R₁, R₄, and the other of R₂ and R₃ are each independently selected from hydrogen, a solubilizing group, or a C₁-C₄ straight or branched alkyl, wherein at least two of R₁, R₂, R₃ or R₄ are hydrogen;

X is S; and

R₅ is an optionally substituted aryl or an optionally substituted heteroaryl.

In certain embodiments, X is NR₆.

In certain embodiments, R₅ is selected from phenyl, 1H-pyrrolyl, pyridinyl, quinolinyl, quinoxalinyl, thienyl, or furanyl; and wherein R₅ is optionally substituted.

In certain embodiments, R₅ is selected from 5-methylfuran-2-yl, unsubstituted pyridin-4-yl, or unsubstituted thien-2-yl.

In certain embodiments, R₅ is selected from 1-methyl-1H-pyrrol-2-yl, 3,4-dimethoxyphenyl, 3,5-dimethoxyphenyl, 4-methoxyphenyl, 2,4,6-trimethylphenyl, 3-hydroxyphenyl, 2-fluoro-5-methylphenyl, unsubstituted phenyl, 4-dimethylaminophenyl, 3,4-dioxymethylenephenyl, 4-carboxyphenyl, unsubstituted pyridin-3-yl, unsubstituted pyridin-4-yl, unsubstituted quinolinyl, unsubstituted quinoxalinyl, or unsubstituted thien-2-yl.

In another embodiment, sirtuin-modulating compounds of the invention are represented by Structural Formula VIa:

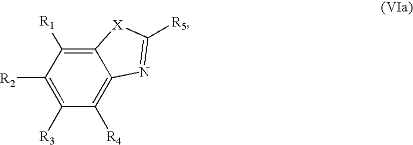

wherein:

one of R₁-R₄ is selected from

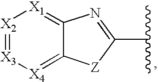

or

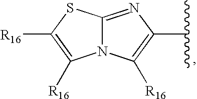

wherein:
one of X₁, X₂, X₃ or X₄ is N and the others are each CR₁₆;
Z is S; and
each R₁₆ is independently selected from hydrogen, a solubilizing group, or a C₁-C₄ straight or branched alkyl, wherein no more than one R₁₆ is selected from a solubilizing group, or a C₁-C₄ straight or branched alkyl;

the remainder of R₁-R₄ are each independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or heteroaryl.

In certain embodiments, X is NR₆.

In certain embodiments, R₅ is selected from phenyl, 1H-pyrrolyl, pyridinyl, quinolinyl, quinoxalinyl, thienyl, or furanyl; and wherein R₅ is optionally substituted.

In certain embodiments, R₅ is selected from 5-methylfuran-2-yl, unsubstituted pyridin-4-yl, or unsubstituted thien-2-yl.

In certain embodiments, R₅ is selected from 1-methyl-1H-pyrrol-2-yl, 3,4-dimethoxyphenyl, 3,5-dimethoxyphenyl, 4-methoxyphenyl, 2,4,6-trimethylphenyl, 3-hydroxyphenyl, 2-fluoro-5-methylphenyl, unsubstituted phenyl, 4-dimethylaminophenyl, 3,4-dioxymethylenephenyl, 4-carboxyphenyl, unsubstituted pyridin-3-yl, unsubstituted pyridin-4-yl, unsubstituted quinolinyl, unsubstituted quinoxalinyl, or unsubstituted thien-2-yl.

In another embodiment, sirtuin-modulating compounds of the invention are represented by Structural Formula VII:

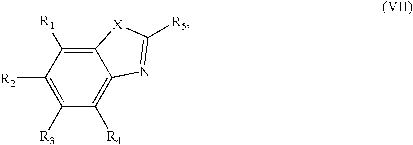

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, X is NR₆.

In certain embodiments, one of R₁ or R₄ is selected from:

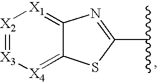

or

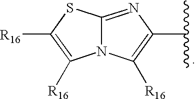

In certain embodiments, R₅ is selected from phenyl, 1H-pyrrolyl, pyridinyl, quinolinyl, quinoxalinyl, thienyl, or furanyl; and wherein R₅ is optionally substituted.

In certain embodiments of formulae I-VII above, the solubilizing group is selected from

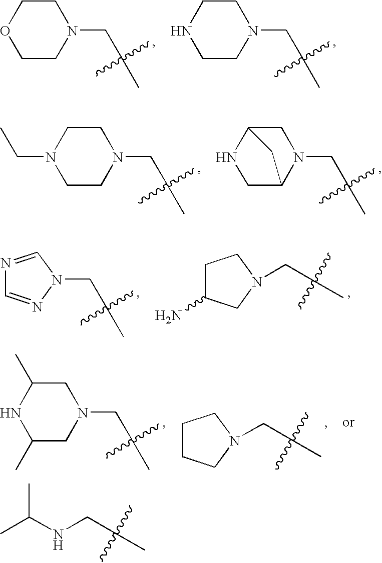

In certain embodiments of formulae I-VII above, R₅ or R₁₅ is selected from

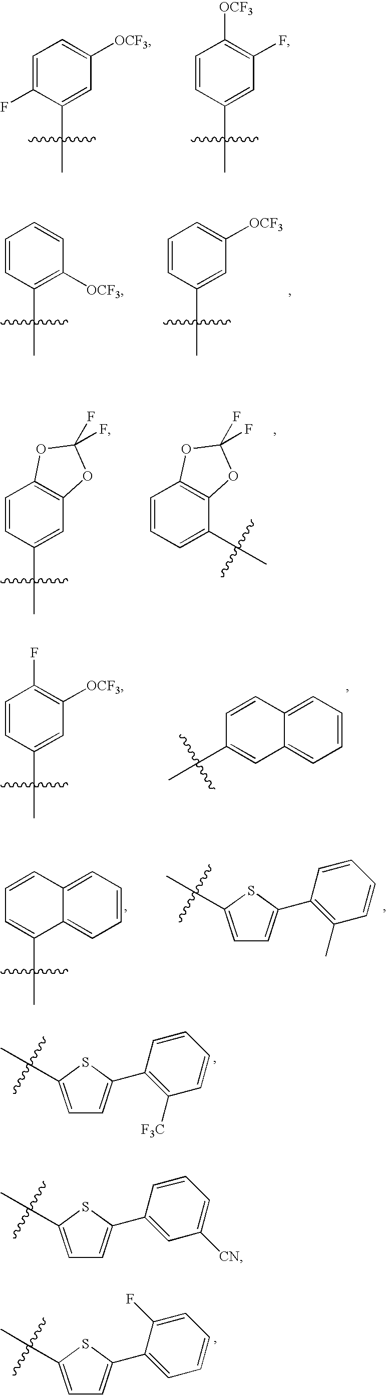

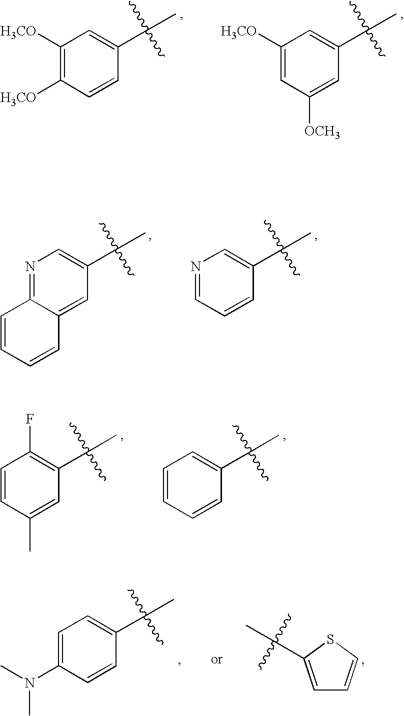

wherein the selection is consistent with the corresponding definition of R₅ or R₁₅ as recited above.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

Also provided are pharmaceutical compositions comprising one or more compounds of the compounds described herein or a salt, prodrug or metabolite thereof. In another aspect, the invention provides methods for using a compound described herein. In certain embodiments, a compound described herein may be used for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, and/or flushing, etc. A compound described herein also be used for treating a disease or disorder in a subject that would benefit from increased mitochondrial activity, for enhancing muscle performance, for increasing muscle ATP levels, or for treating or preventing muscle tissue damage associated with hypoxia or ischemia. In other embodiments, a compound described herein may be used for a variety of therapeutic applications including, for example, increasing cellular sensitivity to stress, increasing apoptosis, treatment of cancer, stimulation of appetite, and/or stimulation of weight gain, etc. As described further below, the methods comprise administering to a subject in need thereof a pharmaceutically effective amount of a compound described herein.

In embodiments of any of the methods, preparations, compounds, compositions, reaction mixtures, the compound or compounds are selected from those described WO 2007/019346, WO 2007/019345, WO 2007/019344, WO 2007/019417, WO 2007/019416, WO 2006/094248, WO 2006/094246, WO 2006/094237, WO 2006/094236, WO 2006/094235, WO 2006/094233, WO 2006/094210, WO 2006/094209, WO 2006/078941, WO 2006/105440, and WO 2008/073451, each of which is hereby incorporated by reference. In other embodiments the compounds are selected from those described as activators of a sirtuin in WO 2007/019346, WO 2007/019345, WO 2007/019344, WO 2007/019417, WO 2007/019416, WO 2006/094248, WO 2006/094246, WO 2006/094237, WO 2006/094236, WO 2006/094235, WO 2006/094233, WO 2006/094210, WO 2006/094209, WO 2006/078941, WO 2006/105440, and WO 2008/073451.

In certain aspects, a compound described herein may be administered alone or in combination with other compounds or other therapeutic agents.

### DETAILED DESCRIPTION

### Definitions

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The term “companion animals” refers to cats and dogs. As used herein, the term “dog(s)” denotes any member of the species Canis familiaris, of which there are a large number of different breeds. The term “cat(s)” refers to a feline animal including domestic cats and other members of the family Felidae, genus Felis. The terms “comprise” and “comprising” are used in the inclusive, open sense, meaning that additional elements may be included.

“Diabetes” refers to high blood sugar or ketoacidosis, as well as chronic, 10 general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

A “direct activator” of a HAT is a molecule that activates a HAT by binding to it. A “direct inhibitor” of a HAT is a molecule inhibits a HAT by binding to it.

The term “ED₅₀” is art-recognized. In certain embodiments, ED₅₀ means the 20 dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “hyperinsulinemia” refers to a state in an individual in which the level of insulin in the blood is higher than normal.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

“Obese” individuals or individuals suffering from obesity are generally individuals having a body mass index (BMI) of at least 25 or greater. Obesity may or may not be associated with insulin resistance.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject”, “individual” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2<nd> ed., Wiley: New York, 1991).

“Replicative lifespan” of a cell refers to the number of daughter cells produced by an individual “mother cell.” “Chronological aging” or “chronological lifespan,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress (for example, heatshock, osmotic stress, high energy radiation, chemically-induced stress, DNA damage, inadequate salt level, inadequate nitrogen level, or inadequate nutrient level). Lifespan can be increased by at least about 20%, 30%, 40%, 50%, 60% or between 20% and 70%, 30% and 60%, 40% and 60% or more using methods described herein.

“HAT-activating compound” refers to a compound that increases the level of a HAT and/or increases at least one activity of a HAT. In an exemplary embodiment, a HAT-activating compound may increase at least one biological activity of a HAT by at least about 10%, 25%, 50%, 75%, 100%, or more. Exemplary biological activities of HAT include acetylation, e.g., of histones and p53; extending lifespan; increasing genomic stability; silencing transcription; and controlling the segregation of oxidized proteins between mother and daughter cells.

“HAT-inhibiting compound” refers to a compound that decreases the level of a HAT and/or decreases at least one activity of a HAT. In an exemplary embodiment, a HAT-inhibiting compound may decrease at least one biological activity of a HAT by at least about 10%, 25%, 50%, 75%, 100%, or more. Exemplary biological activities of HATs include acetylation, e.g., of histones and p53; extending lifespan; increasing genomic stability; silencing; transcription; and controlling the segregation of oxidized proteins between mother and daughter cells.

“HAT-modulating compound” refers to a compound described herein. In exemplary embodiments, a HAT-modulating compound may either up regulate (e.g., activate or stimulate), down regulate (e.g., inhibit or suppress) or otherwise change a functional property or biological activity of a HAT. HAT-modulating compounds may act to modulate a HAT either directly or indirectly. In certain embodiments, a HAT-modulating compound may be a HAT-activating compound or a HAT-inhibiting compound.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

In one embodiment [symbol] -modulating compounds of the invention are represented by Structural Formula (I):

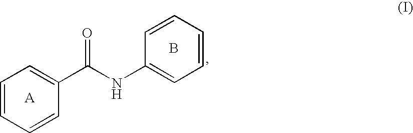

or a salt thereof, where:

Ring A is optionally substituted; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In certain embodiments, Ring B is substituted with at least a carboxy group.

In certain embodiments, Ring B is substituted with at least a substituted or unsubstituted arylcarboxamine, a substituted or unsubstituted aralkylcarboxamine or a polycyclic aryl group.

In certain embodiments, Ring B is substituted with at least a substituted or unsubstituted heteroaryl group or a substituted or unsubstituted heterocyclylcarbonylethenyl group.

In another embodiment, [symbol] modulating compounds of the invention are represented by Structural Formula (II):

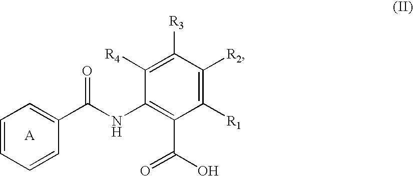

or a salt thereof; where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₄ and —NO₂;

R₅ and R₆ are independently —H, a substituted or unsubstituted alkyl group, a substituted or unsubstituted aryl group or a substituted or unsubstituted heterocyclic group; and

n is 1 or 2.

In a further embodiment, [symbol] -modulating compounds of the invention are represented by Structural Formula (Ia):

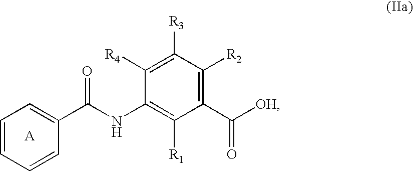

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another embodiment, [symbol] -modulating compounds of the invention are represented by Structural Formula (II):

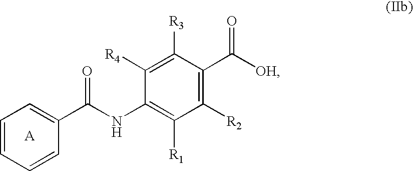

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SRₛ, —OSO₃H, —S(O)R₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ in Structural Formulas (II)-(IIb) are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention provides novel: [symbol] -modulating compounds of Formula (III):

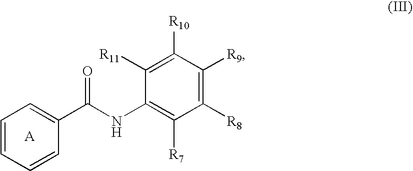

or a salt thereof; where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)OR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy); aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In certain such embodiments, R₈ is selected from thiazolopyridyl, imidazothiazolyl, benzoxazinonyl, or imidazopyridyl.

Particular examples of R₈, where [symbol] indicates attachment to the remainder of Structural Formula (III) include:

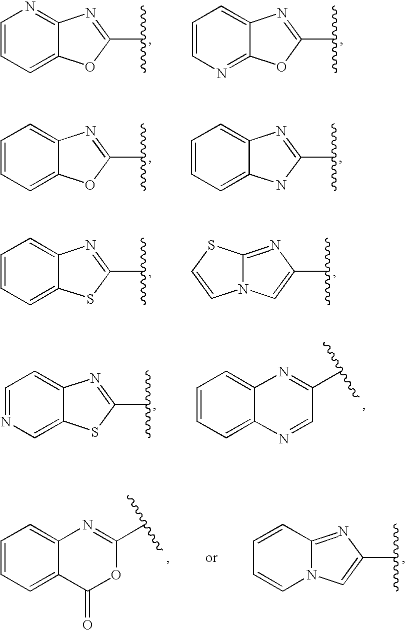

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

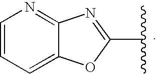

In certain embodiments, R₈ is

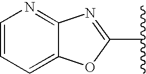

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

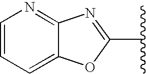

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

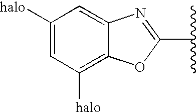

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

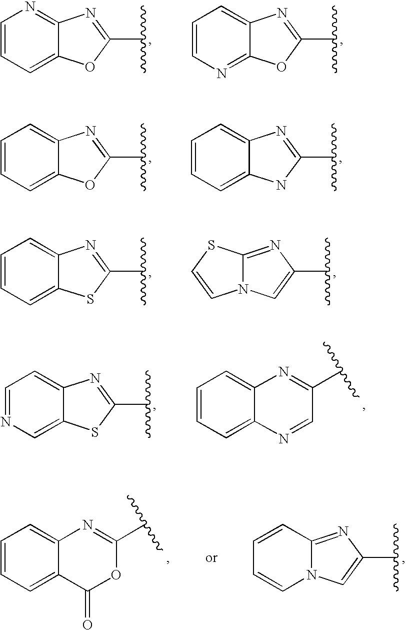

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

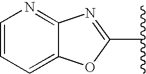

and Ring A is 3-dimethylaminophenyl, none of R₇, R₉, R₁₀ and R₁₁ is —CH₂—N(CH₃)₂ or —C(O)—NH—(CH₂N(CH₃)₂, and/or when R₈ is

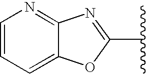

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In yet another aspect, the invention provides novel [symbol] -modulating compounds of Formula (IV):

Ar-L-J-M-K-Ar′ (IV)

or a salt thereof, wherein: each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₁′R₂′; NR₂′C(O)C(O)R₂′; NR₂′C(O)R₂′; NR₂′ (COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or 8, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂; OC(O)R₂; NR₂′R₂′; NR₂′R₃; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′ or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₉′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the invention, each Ar, L, and Ar′ is independently an optionally substituted 5- to 7-membered monocyclic ring system or an optionally substituted 9- to 12-membered bicyclic ring system.

According to another preferred embodiment,
Ar is

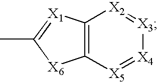

X₁, X₂, X₃, X₄, and X₅ are independently selected from CR₁′ and N; and
X₆ is selected from NR₁′, O, and S;

According to yet another preferred embodiment, X₁ and X₂ are N; X₃, X₄, and X₅ are CR₁′; and X₆ is O.

According to still yet another preferred embodiment, X₁ and X₃ are N; X₂, X₄, and X₅ are CR₁′; and X₆ is O.

According to still yet another preferred embodiment, X₁ and X₄ are N; X₂, X₃, and X₅ are CR₁′; and X₆ is O.

According to still yet another preferred embodiment, X₁ and X₅ are N; X₂, X₃, and X₄ are CR₁′; and X₆ is O.

In another embodiment, the compounds of the formula above are those wherein J is NR₁′, K is absent, and M is C(O).

In yet another embodiment, the compounds of the formula above are those wherein J is absent, K is NR₁′, and M is C(O).

In a further embodiment, compounds of formula (IV) are those where when J is absent and K is NR₁′, M is not C(O) and when J is NR₁′ and K is absent, M is not C(O).

In a preferred embodiment, the compounds above are those wherein L is an optionally substituted 5- to 7-membered carbocyclic or heterocyclic aryl group.

In yet another preferred embodiment, the compounds are those wherein L is an optionally substituted phenylene, pyridinylene, imidazolylene, oxazolylene, or thiazolylene.

In a particularly preferred embodiment, L is an optionally substituted phenylene.

In another particularly preferred embodiment, L is an optionally substituted pyridinylene.

In an even more preferred embodiment, L is phenylene.

In another even more preferred embodiment, L is pyridinylene.

In either of these embodiments, Ar and J may be attached to L at the ortho-, meta-, or para-positions. Particularly preferred are those embodiments where attachment is at the meta-position.

In certain embodiments, L is not phenylene when Ar′ is phenyl. Examples of such embodiments include embodiments where L is an optionally substituted heterocyclic aryl group and Ar′ is an optionally substituted carbocyclic or heterocyclic aryl group, or wherein L is an optionally substituted carbocyclic or heterocyclic aryl group and Ar′ is an optionally substituted heterocyclic aryl group.

In yet another aspect, the invention provides novel [symbol] -modulating compounds of Formula (I) or a salt thereof, wherein

Ring A is substituted with at least one R₁′ group;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group;

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉ ), S(O)₂NR₉′R₉′C(O)R₉′; C2-C10 alkenyl substituted with 1-3 independent R₆′, halo, CF₃, OR₉′, SR₉′, NR₉′R₉′—, COOR₉′, NO₂, CN, C(O)R₉′, C(O)NR₉′R₉′, NHC(O)R₉′, NH(COOR₉′), S(O)NR₉′R₉′, OC(O)R₉′; or R₉′; and

Ring B is substituted with at least one

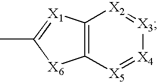

wherein

X₁, X₂, X₃, X₄, and X₅ are independently selected from CR₁′ and N; and

X₆ is selected from NR₁′, O, and S.

In a preferred embodiment, Ring B is phenyl or pyridinyl.

In a further aspect, the invention provides novel [symbol] -modulating compounds of Formula (IVa):

Het-L-Q-Ar′ (IVa)

or a salt thereof, wherein:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—CO)—CR₁′═CR₁′—CR₁′R₁′—, —NR₁′—C(N—CN)—NR₁′—, —NR₁′—C(O)—CR₁′R₁′—,

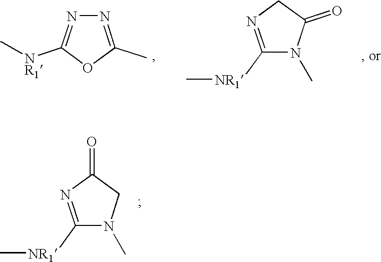

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In certain embodiments, when Het is a polycyclic heteroaryl, L is optionally substituted phenylene, and Ar′ is optionally substituted phenyl; then Q is not —NH—C(O)—.

In certain embodiments, Het and Q are attached to L in a 1-, 2- or 1-,3-configuration (e.g., when L is phenylene, Het and Q are attached in an ortho or a meta orientation). In certain embodiments where Het and Q are attached to L in a 1-,3-configuration, if Het is benzoxazolyl, L is pyridylene and Q is —NH—C(O)—NH, then Ar′ is not 3,4 dioxymethylene phenyl; if Het is methyl thiazolyl, L is phenylene and Q is —NH—C(O)—, then Ar′ is not 3-dimethylamino phenyl; if Het is oxazolopyridyl, L is pyridylene and Q is —NH—C(O)—NH, then Ar′ is not 4-dimethylamino phenyl; if Het is oxazolopyridyl or benzoxazolyl and L is

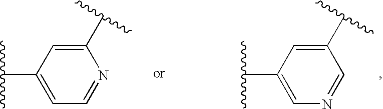

then Q is not —NH—(SO)₂—; and if Het is oxazolopyridyl, L is

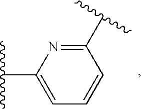

and Q is —NH—C(O)—, then Ar′ is not 3,4 dimethoxyphenyl or pyridyl.

When Het is substituted, it is typically substituted at up to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

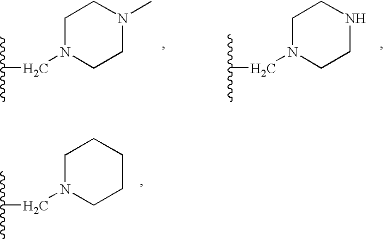

or

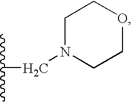

where each R₁₂ is independently selected from optionally substituted C₁-C₃ straight or branched alkyl.

In certain embodiments, Het is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In other embodiments, Het comprises one ring N heteroatom and 1 to 2 additional ring heteroatoms independently selected from N, O or S, such as thiazolyl, triazolyl, oxadiazolyl, thiazolopyridyl, imidazothiazolyl, benzoxazinonyl, or imidazopyridyl.

Particular examples of Het include:

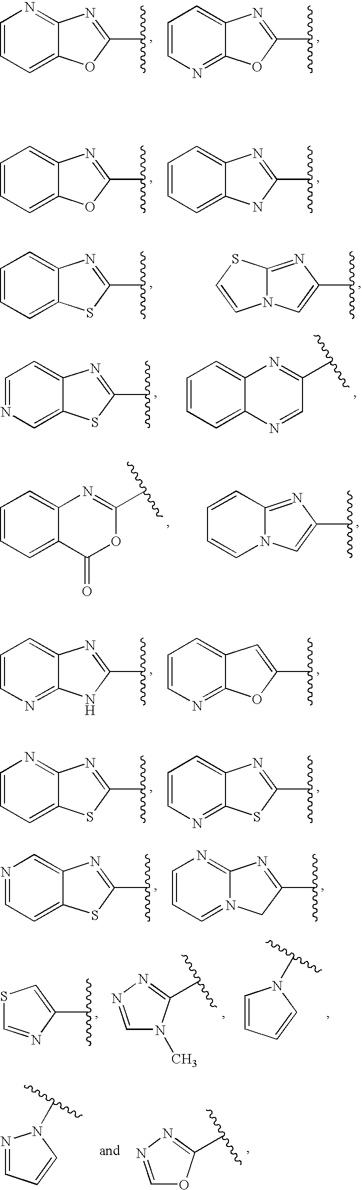

where up to 2 ring carbons not immediately adjacent to the indicated attachment point are independently substituted with optionally substituted C₁-C₃ straight or branched alkyl, phenyl, halo, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

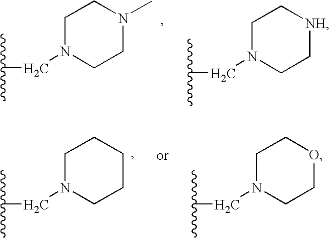

wherein each R₁₂ is independently selected form optionally substituted C₁-C₃ straight or branched alkyl.

In certain embodiments, L is selected from

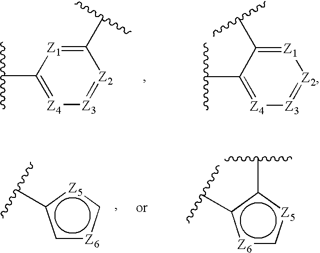

wherein:

each of Z₁, Z₂, Z₃ and Z₄ is independently selected from CH or N, wherein not more than three of said Z₁, Z₂, Z₃ or Z₄ is N;

each of Z₅ and Z₆ is independently selected from C, N, O or S, provided that at least one of Z₅ and Z₆ is N; and

L is optionally substituted at 1 to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

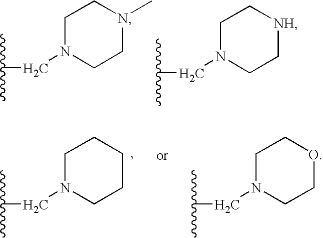

In preferred embodiments, L is selected from phenylene or pyridylene, such as unsubstituted phenylene or phenylene substituted with a single substituent selected from C(O)OCH₃, C(O)OH, CH₂OH, N(CH)₂, or CH₂N(CH₃)₂, or unsubstituted pyridylene.

In certain embodiments, Q is selected from —NH—C(O)—, —NH—S(O)₂—, —NH—C(O)—NH—, —C(O)—NH—, —CH₂—, —N(CH₃)—C(O)—NH—, —NH—C(O)—N(CH₃)—, or —NH—S(O)₂—NH—, particularly —NH—C(O)—, —C(O)—NH—, —NH—, —NH—C(O)—NH, or —NH—S(O)₂—.

In certain embodiments, Ar′ is selected from optionally substituted phenyl, benzothiazolyl, or benzoxazolyl. When Ar′ is phenyl, typical optional substituents are 1 to 3 substituents independently selected from halo, (optionally substituted C₁-C₃ straight or branched alkyl), O-(optionally substituted C₁-C₃ straight or branched alkyl), S-(optionally substituted C₁-C₃ straight or branched alkyl), N(CH₃)₂ or optionally substituted heterocyclyl, or wherein two substituents on adjacent ring atoms are taken together to form a dioxymethylene.

In certain embodiments, Het is selected from

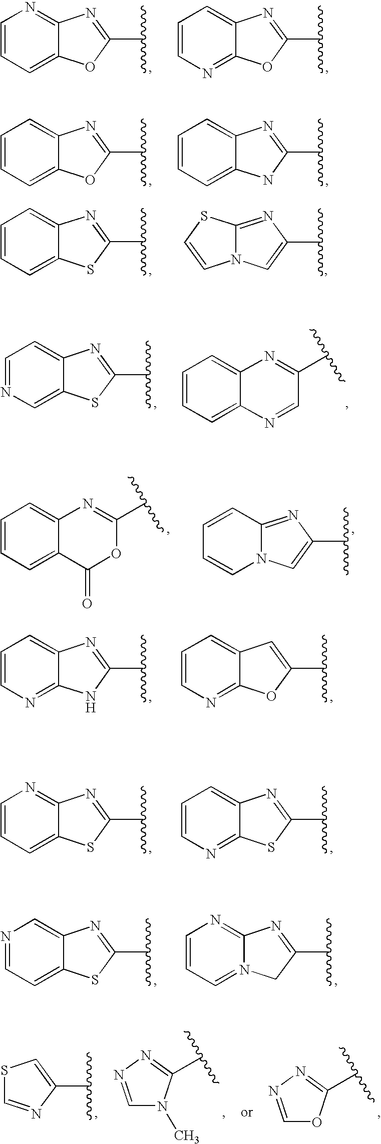

and wherein up to 2 ring carbons not immediately adjacent to the idcated attachment point are independently substituted with optionally substituted C₁-C₃ straight or branched alkyl, phenyl or halo;

L is selected from unsubstituted phenylene, phenylene substituted with a single substituent selected from C(O)OCH₃, C(O)OH, CH₂OH, N(CH₃)₂, or CH₂N(CH₃)₂, or unsubstituted pyridylene;

Q is selected from—NH—C(O)—, —C(O)—NH—, —NH—, —NH—C(O)—NH, or —NH—S(O)₂—; and

Ar′ is selected from optionally substituted phenyl, benzothiazolyl, or benzoxazolyl, wherein said phenyl is optionally substituted with 1 to 3 substituents independently selected from chloro, methyl, O-methyl, S-methyl, N(CH₃)₂, morpholino, or 3,4 dioxymethylene.

In certain embodiments, Q is selected from —NH—C(O)—, —C(O)—NH—, —NH— or —NH—C(O)—NH.

In certain embodiments, the substituents on Ar′ are selected from chloro, methyl, O-methyl, S-methyl or N(CH₃)₂. In certain embodiments, the only substituent on Ar′ is an O-methyl group, particularly an O-methyl group ortho or meta to Q. In certain embodiments, when there are two or more O-methyl groups or Ar′, at least one is ortho or meta to Q.

In certain embodiments, L is pyridyl and Het and Q are at the 1,3- or 2,4-position with respect to the pyridyl nitrogen atom. In certain such embodiments, Q is —NH—S(O)₂—.

In certain embodiments where L is further substituted, the substituent is typically meta to both Het and Q.

In certain embodiments, Q is —NH— and Het is thiazolyl or oxazolopyridyl.

In certain embodiments, Q is —NH— and Ar is benzothiazolyl or benzoxazolyl.

In certain embodim such as when the sirtuin modulator is a sirtuin activator, L is

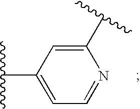

and Q is —NH—(SO)₂—. In certain such embodiments, Het is oxazolopyndyl. When L, Q and optionally Het have these values, Ar′ is advantageously naphthyl or phenyl, where Ar′ is optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments, L is

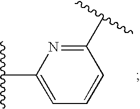

and Q is —NH—C(O)—. In certain such embodiments, Het is oxazolopyridyl. When L, Q and optionally Het have these values, Ar′ is advantageously pyridyl or phenyl optionally substituted with 1 to 3 substituents independently selected from CN, halo, (C₁-C₃ straight or branched alkyl), O—(C₁-C₃ straight or branched alkyl), N(C₁-C₃ straight or branched alkyl)2, or a 5 to 6-membered heterocycle.

In certain embodiments,

, Het comprises one N heteroatom and 1 to 2 additional heteroatoms independently selected from N, O or S;

L is

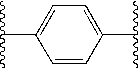

and is optionally substituted;

Q is —NH—C(O)—; and

Ar′ is phenyl substituted with 1 to 3 substituents independently selected from CN, halo, C₁-C₃ straight or branched alkyl, O—(C₁-C₃ straight or branched alkyl), N(C₁-C₃ straight or branched alkyl)₂, or a 5 to 6-membered heterocycle, wherein when R₈ is unsubstituted

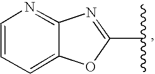

then ring A is:
a) not simultaneously substituted at the 2- and 6-positions with O—(C₁-C₃ straight or branched alkyl);
b) not simultaneously substituted at the 2-position with C₁-C₃ straight or branched alkyl or O—(C₁-C₃ straight or branched alkyl) and at the 3-position with O—(C₁-C₃ straight or branched alkyl);
c) not substituted at the 4-position with O—(C₁-C₃ straight or branched alkyl) unless simultaneously substituted at the 3-position with halo or O—(C₁-C₃ straight or branched alkyl) and unsubstituted at all other positions;
   not substituted at the 4-position with N(C₁-C₃ straight or branched alkyl)₂, or said 5 to 6-membered heterocycle. In certain such embodiments, L is unsubstituted and/or Het is oxazolopyridyl.

In yet another aspect, the invention provides novel [symbol] -modulating compounds of Formula (V):

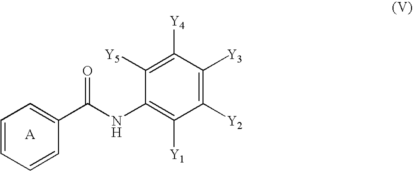

or a salt thereof, wherein:

Ring A is optionally substituted with at least one R₁′ group;

Y₁, Y₂, Y₃, Y₄, and Y₅ are independently R₁′;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′C(O)R₉′; N(R₉′)(COOR₉′); N(R₉′S(O)₂R₉′; S(O)₂NR₉′R₉′; OC(O)R₉′; NR₉′C(O)NR₉′R_{R}′; NR₉′C(O)C(O)R₉′; NR₉′C(O)R₆′; NR₉′S(O)₂NR₉′R₉′; NR₉′S(O)₂R₉′; NR₉′C(O)C(O)NR₉′R₉′; C1-C10 alkyl substituted with 1-3 independent R₆′, halo, CF₃, OR₉′, SR₉′, NR₉′ R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′; COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the above compound, either Y₂ or Y₃ is

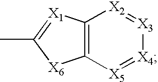

X₁, X₂, X₃, X₄, and X₅ are independently selected from CR₁′ and N; and

X₆ is selected from NR₁′, O, and S.

According to an even more preferred embodiment, X₁ and X₂ are N; X₃, X₄, and X₅ are CR₁′; and X₆ is O.

According to another even more preferred embodiment, X₁ and X₃ are N; X₂, X₄, and X₅ are CR₁′; and X₆ is O.

According to another even more preferred embodiment, X₁ and X₄ are N; X₂, X₃, and X₅ are CR₁′; and X₆ is O.

According to another even more preferred embodiment, X₁ and X₅ are N; X₂, X₃, and X₄ are CR₁′; and Xs is O.

In another aspect, the invention provides [symbol] -modulating compounds of Structural Formula (VII):

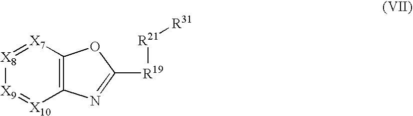

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

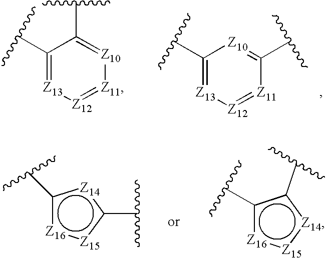

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Zo₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

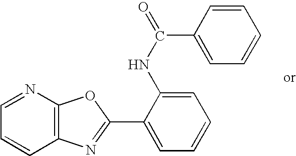

that when R¹⁹ is

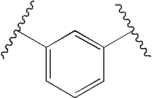

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

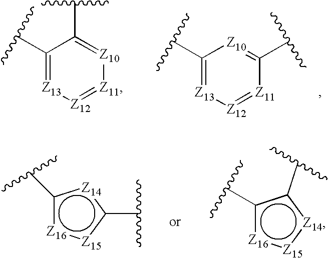

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ sand Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

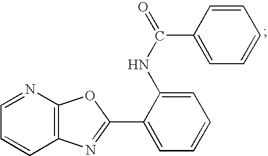

and

when X₈ and X₉ are each independently selected from CR²⁰ or CR₁′, R¹⁹ is

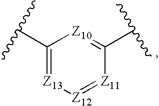

and each of Z₁₀, Z₁₁, Z₁₂ and Z₁₃ is independently selected from CR²⁰, or CR₁′, then:

a) at least one of X₈ and X₉ is not CH; or

b) at least one of Z₁₀, Z₁₁, Z₁₂ and Z₁₃ is CR²⁰, wherein R²⁰ is a solubilizing group.

In certain embodiments, when Z₁₂ is CR²⁰ and R²⁰ is a solubilizing group, the solubilizing group is not —C(O)OCH₂CH₃, —COOH,

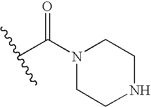

or

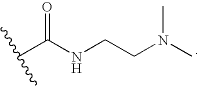

In certain embodiments, when X₈ and X₉ are each independently selected from CR²⁰ or CR₁′, R¹⁹ is

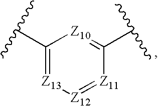

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁ and Z₁₃ is CR²⁰, wherein R²⁰ is a solubilizing group.

In certain embodiments, when R¹⁹ is

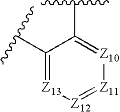

and each of Z₁₀, Z₁₁, Z₁₂ and Z₁₃ is CR²⁰, or CR₁′; X₈ and X₉ are CR²⁰ or CR₁′; R²¹ is —NHC(O)—; and R³¹ is optionally substituted phenyl, then R³¹ is a substituted phenyl, at least one R₁′ in a CR₁′ moiety is optionally substituted C₁-C₃ straight or branched alkyl, or at least one R²⁰ in a CR²⁰ is a solubilizing group, or a combination thereof.

In certain embodiments, R¹⁹ is selected from phenyl, pyridyl, thienyl or furyl.

In certain embodiments, R¹⁹ is

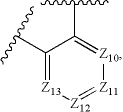

wherein each of Z₁₀, Z₁₁, Z₁₂ and Z₁₃ is independently selected from CR²⁰ or CR₁′; and

R²¹ is —NH—C(O)—; and

R³¹ is a substituted phenyl.

In certain such embodiments, when X₉ is N, R³¹ is not 2,4 dimethoxyphenyl and/or when X₁₀ is N, R³¹ is not halo substituted phenyl; 3,4-dioxoethylenephenyl; or 3,5-dimethoxyphenyl.

In preferred embodiments, R³¹ is optionally substituted with 1 to 3 substituents independently selected from —OCH₃, —CH₃, —N(CH₃)₂, pyrazinoxy or a solubilizing group. Suitable examples of R³¹ include 3-methoxy-4-((4-methylpiperazin-1-yl)methyl)phenyl, 3-methoxy-4-morpholinomethylphenyl, 3-methoxy-4-diaminomethylphenyl, 3-methoxy-4-((pyrrolidin-1-yl)methyl)phenyl, 3,4-dimethoxyphenyl, 3,5-dimethoxyphenyl, 2,3,4-trimethoxyphenyl, 3,4,5-trimethoxyphenyl, 2-dimethylaminophenyl, 3-dimethylaminophenyl, 4-dimethylaminophenyl, or 3,5-dimethylphenyl.

In certain embodiments, R¹⁹ is selected from

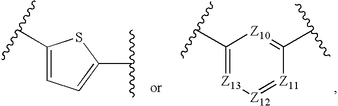

wherein one of Z₁₀, Z₁₁, Z₁₂, and Z₁₃ is N and the others are independently selected from CR²⁰ or CR₁′;

R²¹ is selected from —NH—, —NH—C(O)—, —NH—C(O)—NH, —NH—C(S)—NH— or —NH—S(O)₂—; and

R³¹ is selected from an optionally substituted phenyl, an optionally substituted naphthyl, or an optionally substituted heteroaryl.

In certain such embodiments,
a) when R²¹ is —NH—S(O)₂—, either:
   i) Z₁₀ is N; or
   ii) Z₁₁ is N and R³¹ is halophenyl or 2-methoxy-5-methylphenyl;
b) when R¹⁹ is

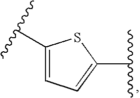

R³¹ is not 4-dimethylaminophenyl, 2,3,4-trimethoxyphenyl, or 3,5 dimethoxyphenyl; and/or
c) when R²¹ is —NH—C(O)—NH— and Z₁₀ is N, R³¹ is not 4-dimethylaminophenyl.

In certain such embodiments, R³¹ is selected from optionally substituted phenyl, benzothiazolyl, or benzoxazolyl.

In yet another embodiment, the invention provides: [symbol] -modulating compounds of Structural Formula (VIII):

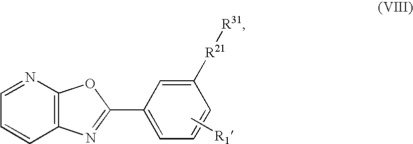

or a salt thereof wherein:

R₁′ is selected from H or optionally substituted C₁-C₃ straight or branched alkyl;

R²¹ is selected from —NR₁′—C(O)—, —NR₁′-S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁—S(O)₂—, —NR₁′—, —CR₁′R₁′—, —NR₁′—C(O)—CR₁—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

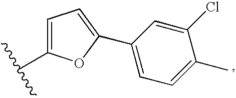

1-methoxynaphthyl, 2-methoxynaphthyl, or unsubstituted 2-thienyl;

when R₁′ is methyl, and R²¹ is —NH—C(O)—CH═CH—, R³¹ is not

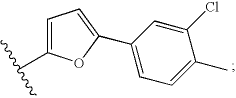

when R₁′ is methyl, and R²¹ is —NH—C(O)—CH—O—, R³¹ is not unsubstituted naphthyl, 2-methoxy, 4-nitrophenyl, 4-chloro-2-methylphenyl, or 4-t-butylphenyl; and

when R²¹ is —NH—C(O)—, R³¹ is not optionally substituted phenyl.

In certain embodiments, R²¹ is —NH—C(O)—; and R³¹ is phenyl optionally substituted with 1 to 3 substituents independently selected from —OCH₃, —CH₃, —N(CH₃)₂, or a solubilizing group.

In certain such embodiments, R²¹ is —NH—C(O)— and R³¹ is selected from unsubstituted phenyl, 2-methoxyphenyl, 3-methoxyphenyl, 2,3,4-trimethoxyphenyl, 3,4,5-trimethoxyphenyl, 2,4-dimethoxyphenyl, 3,5-dimethoxyphenyl, 2-methyl-3-methoxyphenyl, 2-morpholinophenyl, 2-methoxy-4-methylphenyl, 2-dimethylaminophenyl, 4-dimethylaminophenyl, or

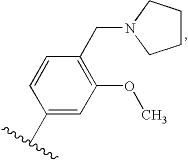

particularly phenyl; 2-methoxyphenyl; 3-methoxyphenyl; 2,3,4-trimethoxyphenyl; 3,4,5-trimethoxyphenyl; 2,4-dimethoxyphenyl; 3,5-dimethoxyphenyl; 2-methyl-3-methoxyphenyl; 2-morpholinophenyl; 2-methoxy-4-methylphenyl; 2-dimethylaminophenyl; or 4-dimethylaminophenyl.

In a further embodiment, the invention provides [symbol] -modulating compounds of Structural Formula (IX):

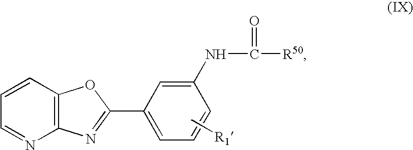

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

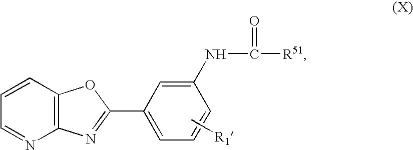

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo[b]thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In certain embodiments, R⁵¹ is selected from pyrazolyl, thiazolyl, oxazolyl, pyrimidinyl, furyl, thienyl, pyridyl, isoxazolyl, indolyl, benzopyrazolyl, benzothiazolyl, benzoxazolyl, quinoxalinyl, benzofuranyl, benzothienyl, quinolinyl, benzoisoxazolyl, benzotriazinyl, triazinyl, naphthyl, or

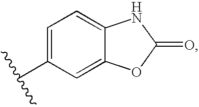

and wherein R⁵¹ is optionally substituted. In certain such embodiments, R⁵¹ is selected from pyrazolyl, thiazolyl, oxazolyl, pyrimidinyl, indolyl, pyrazinyl, triazinyl, or

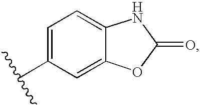

and R⁵¹ is optionally substituted.

In another aspect, the invention provides [symbol] modulating compounds of Structural Formula (XI):

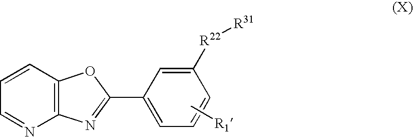

or a salt thereof, wherein:

R₁′ is selected from H or optionally substituted C₁-C₃ straight or branched alkyl;

R²² is selected from —NR₂₃—C(O)—, —NR₁′—S(O)₂—, —NR₁′—C(O)—NR₁—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′S(O)₂—NR₁′—, —NR₁′C(O)—NR₁′S(O)₂—, —NR₁′—CR₁′R₁′—C(O)—NR₁′—, —CR₁′R₁′—C(O)—NR₁′, —NR₁′—C(O)—CR₁′═CR₁′—CR₁′R₁′—, —NR₁′—C(═N—CN)—NR₁′—, —NR₁′—C(O)—CR₁′R₁—O—, —NR₁′—C(O)—CR₁′R₁—CR₁′R₁′—O—, —NR₁′—S(O)₂—CR₁′R₁′—, —NR₁′—S(O)₂—CR₁′R₁—CR₁′R₁′—, or —NR₁′—C(O)—CR₁′R₁′—, wherein R₂₃ is an optionally substituted C₁-C₃ straight or branched alkyl; and

R³¹ is selected from an Optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R₂₂ is —NH—C(O)—CH₂—, R³¹ is not 3,4-dimethoxyphenyl, 4-chlorophenyl, or unsubstituted phenyl;

when R²² is —NH—(O)—CH₂—O—R³¹ is not 2,4-dimethyl-6-nitrophenyl, 2- or 4-nitrophenyl, 4-cyclohexylphenyl, 4-methoxyphenyl unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂, R³¹ is not unsubstituted phenyl.

In certain embodiments, R²² is selected from —C(O)—NH—, —NH—, or —CC(O)—NH—CH₃.

In certain embodiments, such as when R²² is selected from —C(O)—NH—, —NH—, or —C(O)—NH—CH₃, R³¹ is selected from optionally substituted phenyl, benzothiazolyl, quinoxalinyl, or benzoxazolyl.

In yet another aspect, the invention provides [symbol] -modulating compounds of Structural Formula (XII):

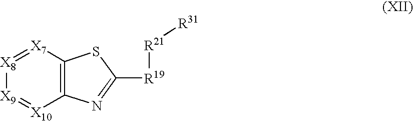

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

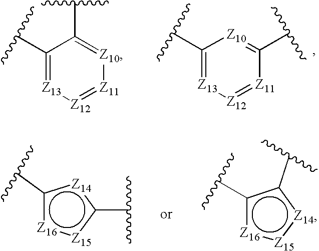

wherein:
each Z₁₀, Z₁ₗ, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that when R₁₉ is

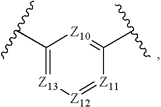

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

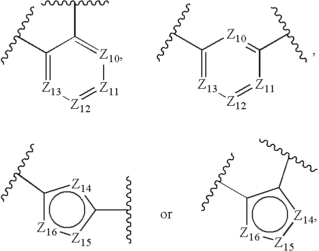

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or heteroaryl, with the proviso that:

when X₇ is N, R¹⁹ is

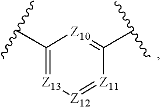

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In certain embodiments, R²¹ is —NH—C(O)— and R¹⁹ is selected from:

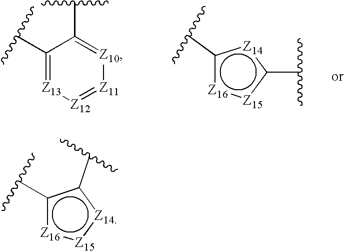

In certain embodiments, R¹⁹ is selected from optionally substituted phenyl, optionally substituted pyridyl, optionally substituted thienyl or optionally substituted furyl.

In certain embodiments, R¹⁹ is

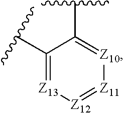

wherein each of Z₁₀, Z₁₁, Z₁₂ and Z₁₃ is independently selected from CR²⁰ or CR₁′; and

R²¹ is selected from —NH—C(O)—, —NH—C(O)—CH(CH₃)—O—, —NH—C(O)—CH₂—O—, or —NH—S(O)₂—CH₂—CH₂—; and

R³¹ is selected from an optionally substituted aryl, or an optionally substituted heteroaryl.

In certain such embodiments, R³¹ is optionally substituted with 1 to 3 substituents independently selected from —OCH₃, —CH₃, —N(CH₃)₂, phenyl, phenoxy, 3,4-dioxymethylene, fluoro, or another solubilizing group. Suitable examples of R³¹ include unsubstituted quinolinyl, 2,4-dimethoxyphenyl, 3,4-dimethoxyphenyl, 3,5-dimethoxyphenyl, 3,4,5-trimethoxyphenyl, 2,3,4-trimethoxyphenyl, 2-dimethylaminophenyl, 3-dimethylaminophenyl, 4-dimethylaminophenyl, 3,5-dimethylphenyl, 3,5-difluorophenyl, 3-trifluoromethoxyphenyl, unsubstituted quinoxalinyl, unsubstituted benzopyrimidinyl, quinoxalinyl, unsubstituted benzopyrimidinyl,

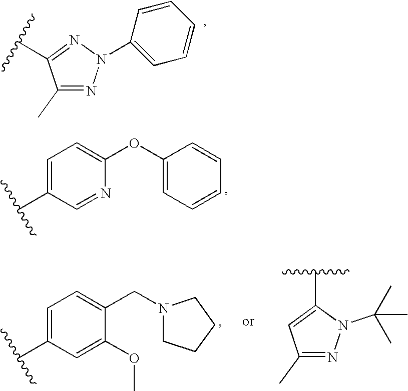

In certain such embodiments, R³¹ is not phenyl-substituted furyl.

In certain embodiments, R¹⁹ is selected from

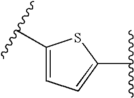

or

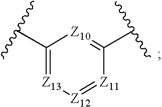

each of Z₁₀, Z₁₁, Z₁₂ and Z₁₃ is independently selected from CR²⁰, or CR₁′,

R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

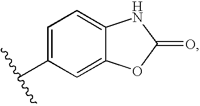

and R³¹ is optionally substituted (e.g., optionally substituted with up to three substituents independently selected from —OCH₃, —CH₃, —N(CH₃)₂, —O-phenyl, or another solubilizing group). Suitable examples of R³¹ include unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

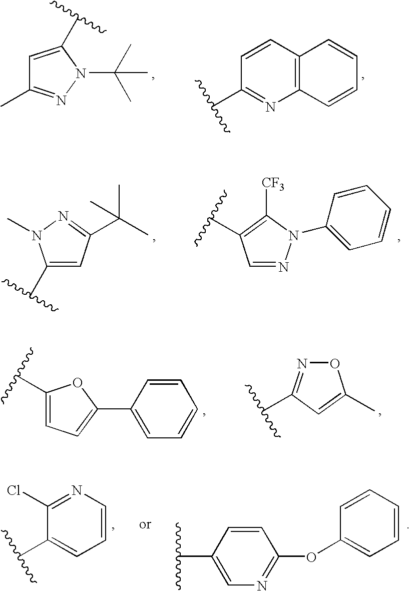

In certain embodiments, one or more of the following conditions applies:

when Xs is N, R²¹ is —NH—C(S)—NH—, and R¹⁹ is phenyl, R³¹ is not 2-methoxy-5-nitrophenyl, 2-S-methylphenyl or 2-acetylphenyl;

when X₈ is N, R²¹ is —NH—S(O)₂—, and R¹⁹ is phenyl, R³¹ is not thiadiazole-substituted thienyl or 4-methylsulfonylphenyl;

when X₈ is N, R²¹ is —NH—CO—, and R¹⁹ is phenyl, R³¹ is not 2,4-difluorophenyl, pyridyl-substituted thienyl, 3,4-dichlorophenyl, 4-t-butylphenyl, or 3-benzyloxyphenyl;

when X₉ is N, R²¹ is —NH—C(O)— and R¹⁹ is

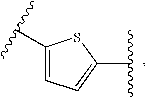

R³¹ is not 2,3,4-trimethoxyphenyl or 3,5-dimethoxyphenyl; and

when X₉ is N, R²¹ is —NH—C(O)— and R¹⁹ is phenyl, R³¹ is not 3,5-dimethoxyphenyl.

In a further embodiment, the invention provides compounds of Structural Formula (XIII):

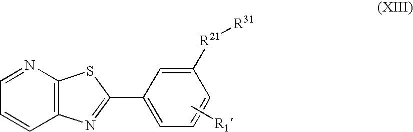

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′↑CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In certain embodiments, R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from a monocyclic or bicyclic aryl or a monocyclic or bicyclic heteroaryl, and comprises a solubilizing group substituent.

In certain embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

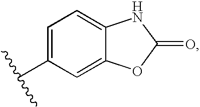

and R³¹ is optionally substituted.

In certain embodiments, R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, particularly when R²¹ is —NH—C(O)—, R³¹ is selected from R³¹ is selected from unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

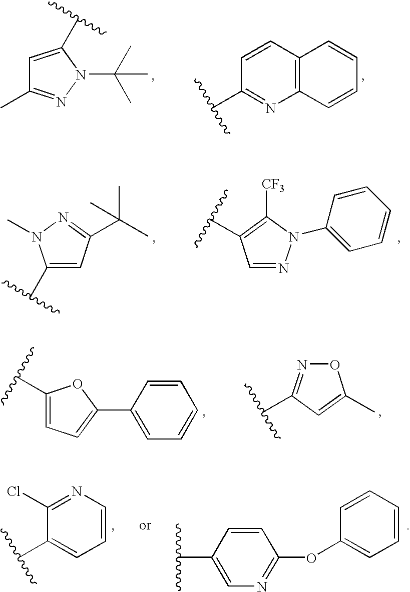

In one aspect, the invention provides [symbol] -modulating compounds of Structural Formula (XIV):

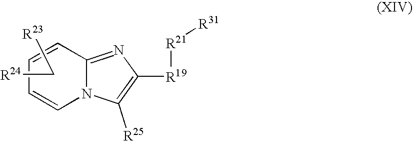

or a salt thereof, wherein:

each of R²³ and R²⁴ is independently selected from H, —CH₃ or a solubilizing group;

R²⁵ is selected from H or a solubilizing group; and

R¹⁹ is selected from:

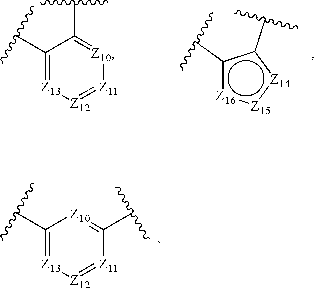

or

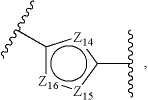

wherein:
each Z₁₀, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁₁—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

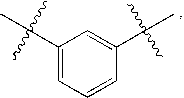

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In certain embodiments, each of R²³ and R²⁴ is independently selected from H, —CH₃ or a solubilizing group;

R²⁵ is selected from H, or a solubilizing group; and

R¹⁹ is selected from:

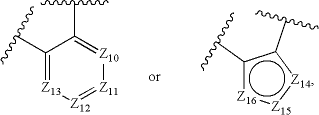

wherein:
each Z₁₀, Z₁₁, Z₁₂ and Z₁₃ is independently selected from N, CR²⁰; or CR₁′; and
each Z₁₄, Z₁₅ and Z₁₆ is independently selected from N, NR₁′, S, O, CR₂, or CR₁′,

wherein:

zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁—CR₁′—CR₁′R₁′—, —NR₁′—C(═N—CN)—NR₁′, —NR₁′—C(O)—CR₁′R₁′—O—, —NR₁′—C(O)—CR₁′R₁—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, R³¹ is not 2,4-dimethoxyphenyl.

Typically, R²⁵ is selected from H, —CH₂—N(CH₃)₂, or

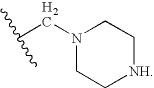

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In certain embodiments, each of R²³ and R²⁴ is independently selected from H, —CH₃ or a solubilizing group;

R²⁵ is selected from H, or a solubilizing group; and

R¹⁹ is selected from:

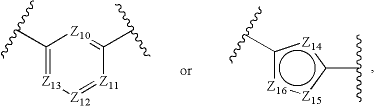

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ is N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is phenyl, at least one of R²³, R²⁴, or R²⁵ is a solubilizing group and wherein said compound is not 2-chloro-N-[3-[3-(cyclohexylamino)imidazo[1,2-a]pyridin-2-yl]phenyl]-4-nitrobenzamide.

Typically, R²⁵ is selected from H, —CH₂—N(CH₃)₂, or

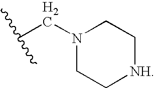

Typically, R²³ and R²⁴ are H.

Typically, R¹⁹ is selected from phenyl, pyridyl, thienyl or furyl, particularly optionally substituted phenyl. Preferably, a phenyl is optionally substituted with:

b) up to three —O—CH₃ groups; or

b) one —N(CH₃)₂ group.

In another aspect, the invention provides [symbol] -modulating compounds of Structural Formula (XV):

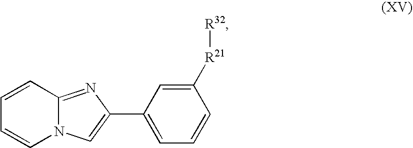

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′, —C(O)—NR₁′—, —CR₁′R₁′—C(O)—NR₁′—, —NR₁′—C(O)—CR₁′—CR₁′R₁′—CR₁′R₁′—, —NR₁′—C(═N—CN)—NR₁′—NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

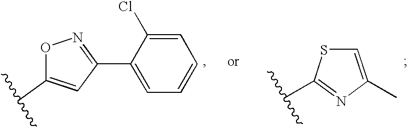

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

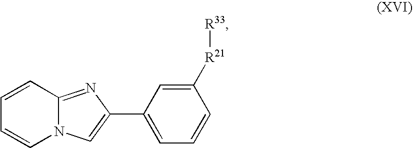

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, —NR₁′—C(═N—CN)—NR₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In certain embodiments, R²¹ is selected from —NR²²—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, —NR₁′—C(═N—CN)—NR₁′—, or —NR₁′—C(O)—CR₁′R₁′—; and

each R₁′ is independently selected from H or optionally substituted C₁-C₃ straight or branched alkyl;

R²² is an optionally substituted C₁-C₃ straight or branched alkyl; and

R³³ is phenyl comprising a solubilizing group substituent, wherein: when R²¹ is —NH—S(O)₂ said phenyl comprises an additional substituent.

In certain embodiments, R²¹ is selected from —NR²²—C(O)—, —NR₁′—C(S)—NR₁′—, —NR₁′—C(S)—NR₁′—CR₁′R₁′—, —NR₁′—C(O)—CR₁′R₁′—NR₁′—NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′—CR₁′—CR₁′R₁′—, or —NR₁′—C(═N—CN)—NR₁′—,

each R₁′ is independently selected from H or optionally substituted C₁-C₃ straight or branched alkyl; and

R²² is an optionally substituted C₁-C₃ straight or branched alkyl.

In certain embodiments, R³³ is optionally substituted on up to three carbon atoms with a substituent independently selected from —O—CH₃, —CH₃, —N(CH₃)₂, —S(CH₃), or CN; or substituted on adjacent carbon atoms with

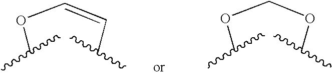

bridging said adjacent carbon atoms.

In a further embodiment, the invention provides [symbol] -modulating compounds of Structural Formula (XVII):

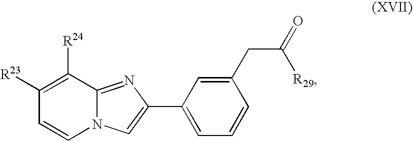

or a salt thereof wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R² is CH₃, one —O—CH₃ group at the 2 or 3 position,

wherein R²⁹ is optionally additionally substituted with a solubilizing group.

In certain embodiments, R²⁹ is phenyl substituted with:

a) three —O—CH₃ groups located at the 2, 3 and 4 positions; or

b) one —N(CH₃)₂ group.

In one aspect, the invention provides [symbol] -modulating compounds of Structural Formula (XVIII):

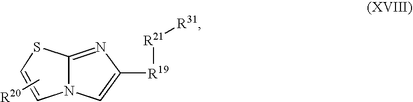

or a salt thereof, wherein

R¹⁹ is selected from:

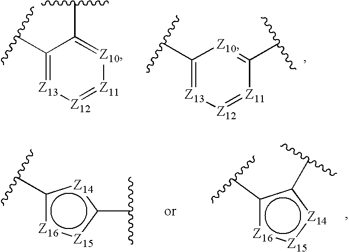

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄ Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—S(O)₂—CR₁′R₁′—, —NR₁′—S(O)₂—CR₁′R₁—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

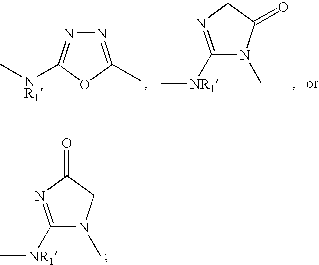

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

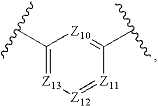

Z₁₀, Z₁₁, Z₁₂ and Z₁₃ are each CH, R²⁰ is H, and R²¹ is —NHC(O)—, R³¹ is not an optionally substituted phenyl.

In certain embodiments, R¹⁹ is selected from:

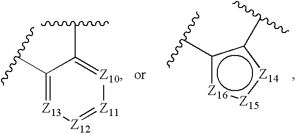

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′≡CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

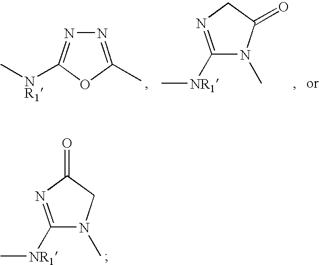

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, compounds of Structural Formula (XVIII) have the formula:

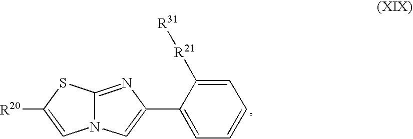

or a salt thereof, wherein

R²⁰ is selected from H or a solubilizing group;

R²¹ is selected from —NH—C(O)—; or —NH—C(O)—CH₂—; and

R³¹ is selected from an optionally substituted monocyclic or bicyclic aryl, or an optionally substituted monocyclic or bicyclic heteroaryl.

Typically, R¹⁹ in compounds of Structural Formula (XVIII) is selected from phenyl, pyridyl, thienyl or furyl, particularly optionally substituted phenyl.

Typically, R²⁰ is selected from H, —CH₂—N(CH₃)₂,

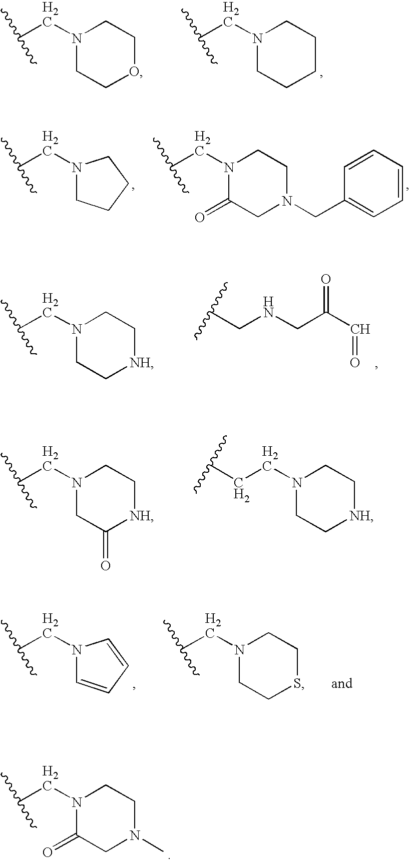

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In certain such embodiments, when R²¹ is —NR₁′—C(O)—, R³¹ is not 4-cyanophenyl or

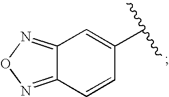

and/or when R²¹ is —NR₁′—S(O)₂—, R³¹ is not 4-methoxyphenyl or 4-t-butylphenyl.

In certain such embodiments, when R¹⁹ is

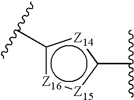

or

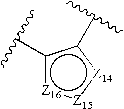

and R²¹ is —NR₁′—C(O)—, R³¹ is not 4-cyanophenyl or

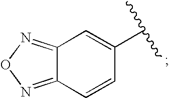

and/or when R¹⁹ is

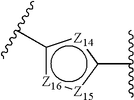

or

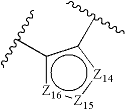

and R²¹ is —NR₁′—S(O)₂—, R³¹ is not 4-methoxyphenyl or 4-t-butylphenyl.

In another aspect, the invention provides [symbol] modulating compounds of Structural Formula (XX):

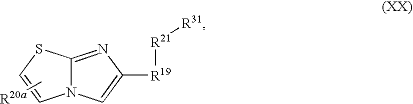

or a salt thereof, wherein

R¹⁹ is selected from:

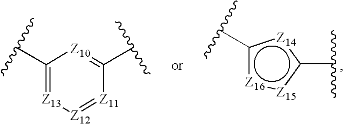

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R²⁰ is independently selected from H or a solubilizing group;

R^{20a} is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; ═NR₁′—C(O)—CR₁′R₁′—CR₁′R₁′—NR₁′—C(O)—R₁′—CR₁′R₁′—CR₁′R₁′—, —NR₁′—C(O)—O—,

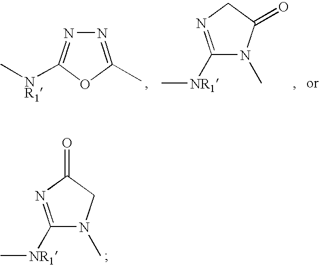

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

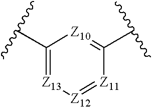

and Z₁₀, Z₁₁, Z₁₂ and Z₁₃ are each CH, R^{20a} is a solubilizing group.

Typically, R¹⁹ in compounds of Structural Formula (XX) is selected from phenyl, pyridyl, thienyl or furyl, particularly optionally substituted phenyl.

Typically, R^{20a} is selected from H, —CH₂—N(CH₃)₂,

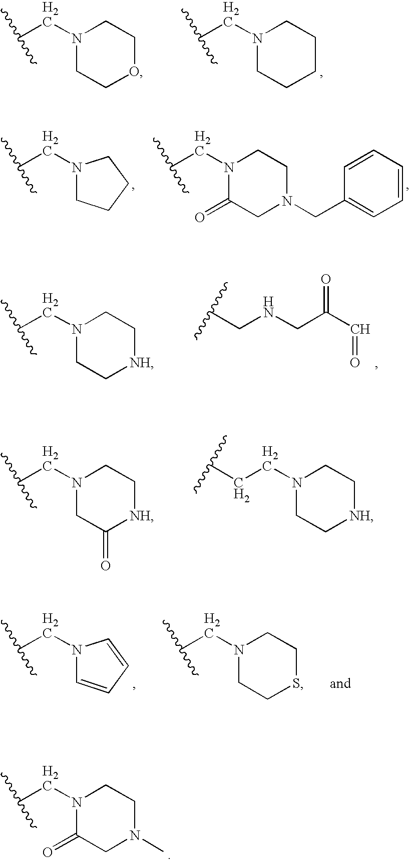

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In yet another aspect, the invention provides [symbol] -modulating compounds of Structural Formula (XXI):

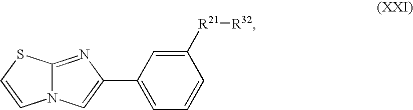

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

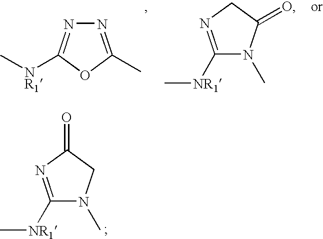

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In certain embodiments, R³² is selected from pyrrolyl, pyrazolyl, pyrazinyl, furyl, pyridyl, pyrimidinyl, or thienyl, and R³² is optionally substituted and is optionally benzofused.

In certain embodiments, R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═—CR₁′—, —NR₁′—S(O)₂—NR₁′—, —NR₁′—C(O)—NR₁′—S(O)₂—, —NR₁′—CR₁′R₁′-C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, CR₁′R₁′—CR₁′R₁′—, —NR₁′—C(O)—O—,

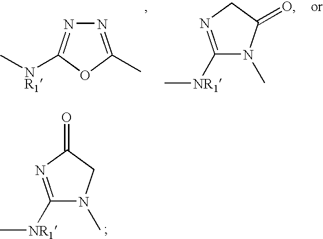

wherein
each R₁′ is independently selected from H or optionally substituted C₁-C₃ straight or branched alkyl; and

R³² is selected from benzofuryl, methylfuryl, benzothienyl, pyridyl, pyrazinyl, pyrimidinyl, pyrazolyl, wherein said methyfuryl, pyridyl, pyrazinyl, pyrimidinyl or pyrazolyl is optionally benzofused and wherein R³² is optionally substituted or further substituted.

In a further aspect, the invention provides [symbol] -modulating compounds of Structural Formula (XXII):

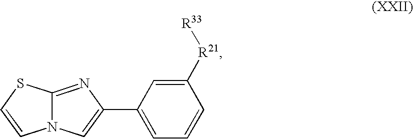

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—C(O)—CR₁′R₁′—O—, —NR₁′—S(O)₂—CR₁′R₁′—, —NR₁′—S(O)₂—CR₁′R₁′—CR₁′R₁′—,

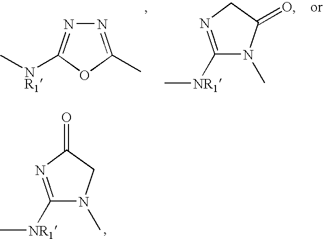

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

Preferably, R²¹ is selected from —NH—C(O)— or —NH—C(O)—CH₂—.

In one aspect, the invention provides [symbol] -modulating compounds of Structural Formula (XXII):

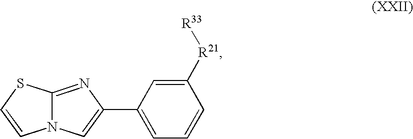

or a salt thereof, wherein:

R²¹ is selected from —NH—C(O)—, or —NH—C(O)—CH₂—; and

R³³ is phenyl substituted with

e) one —N(CH₃)₂ group;

f) one CN group at the 3 position;

g) one —S(CH₃) group; or

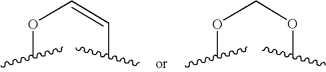

bridging the 3 and 4 positions.

In another aspect, the invention provides [symbol] -modulating compounds of Structural Formula (XXIII):

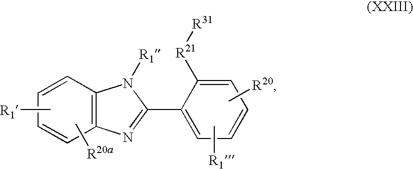

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

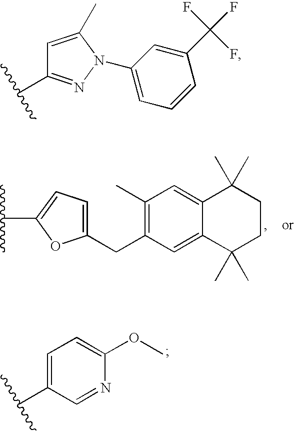

when R²¹ is —NH—C(O)— and each of R²⁰, R^{20a}, R₁′, R₁″ and R₁′″ is hydrogen, R³¹ is not

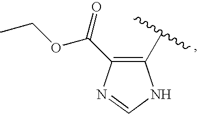

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;
when R²¹ is —NH—C(O)—, R₁″ is methyl; and each of R²⁰, R^{20a}, R₁′ and R₁′″ is hydrogen, R³¹ is not 2-methylaminophenyl,

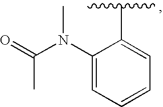

or

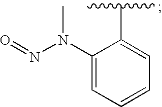

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;
when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

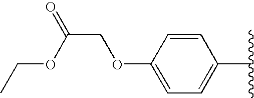

or

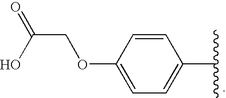

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In a particular aspect, the invention provides [symbol] -modulating compounds of Structural Formula (XXIII):

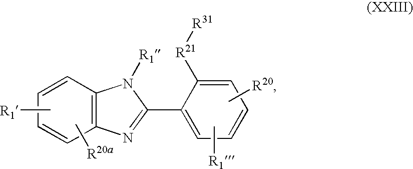

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In yet another aspect, the invention provides [symbol] -modulating compounds of Structural Formula (XXIV):

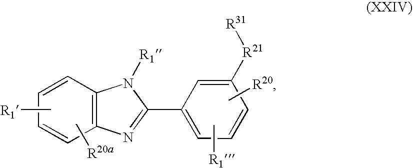

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen,

R³¹ is not unsubstituted phenyl, 4-chlorophenyl, 4-methylphenyl, or 4-acetoamidophenyl;

when R²¹ is —NH—S(O)_{z}—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;

when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

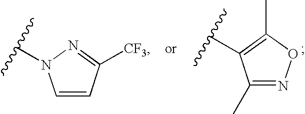

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular, aspect, the invention provides [symbol] -modulating compounds of Structural Formula (XXIV):

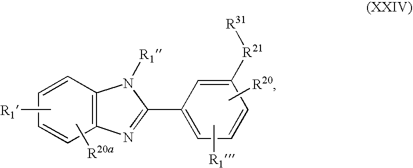

or a salt thereof, wherein:

each R²⁰ and R^{20a} is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In certain embodiments, when R²¹ is —NH—C(O)—CH₂—, R³¹ is not 2-methylphenyl; or 3,4-dimethoxyphenyl; when R²¹ is —NH—C(O)—CH═CH—, R³¹ is not 2-chlorophenyl; and/or when R²¹ is —NH—C(O)—NH—, R³¹ is not unsubstituted benzimidazolyl.

In a further aspect, the invention provides [symbol] -modulating compounds of Structural Formula (XXV):

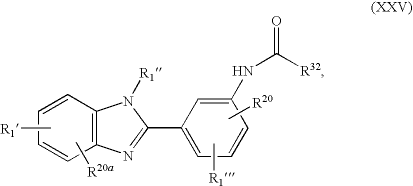

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In certain embodiments, R³² is selected from 3,4-dimethoxyphenyl, 2,6-dimethoxyphenyl, or 2,4-dimethoxyphenyl; wherein R³ is further optionally substituted with a solubilizing group.

In certain embodiments, R³² is not unsubstituted thienyl; unsubstituted phenyl; 2-methylphenyl; 4-fluorophenyl; 4-methoxyphenyl; 4-methylphenyl; 3,4-dioxyethylenephenyl; 3-acetylamino-4-methylphenyl; 3-[(6-amino-1-oxohexyl)amino]-4-methylphenyl; 3-amino-4-methylphenyl; 3,5-dimethoxyphenyl; 3-halo-4-methoxyphenyl; 3-nitro-4-methylphenyl; or 4-propoxyphenyl.

In one aspect, the invention provides [symbol] -modulating compounds of Structural Form (XXIV):

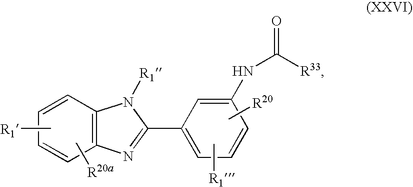

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or,

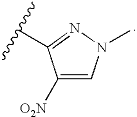

In a particular aspect, the invention provides [symbol] -modulating compounds of Structural Formula (XXVI):

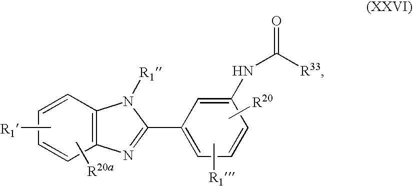

or a salt thereof, wherein:
each R²⁰ and R^{20a} is independently selected from H or a solubilizing group, wherein at least one of R²⁰ or R^{20a} is a solubilizing group;
each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] -modulating compounds of Structural Formula (XXVII):

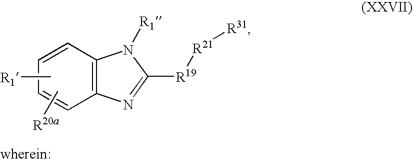

each R²⁰ and R^{20a} is independently selected from H or a solubilizing group;
each R₁′ and R₁″ is independently selected from H or optionally substituted C₁-C₃ straight or branched alkyl;

R¹⁹ is selected from:

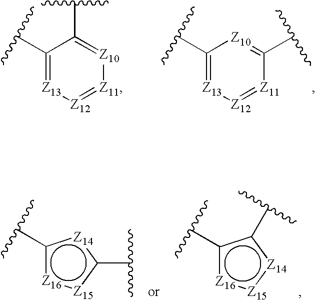

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

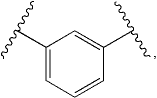

R³¹ is not unsubstituted pyridyl, 2,6-ethoxyphenyl, 3,4,5-trimethoxyphenyl or unsubstituted furyl.

In a particular aspect, the invention provides: [symbol] -modulating compounds of Structural Formula (XXVII):

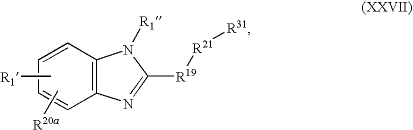

or a salt thereof; wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

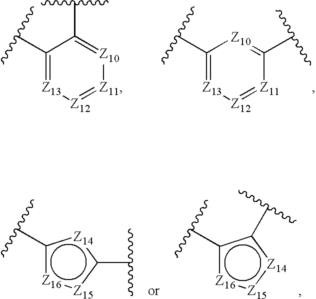

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, R₁′C(═NR₁′)—NR₁′, —C(O)—NR₁′, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

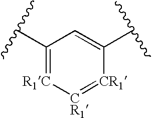

R³¹ is not 2-methylphenyl or 3,4-dimethoxyphenyl;

when R²¹ is —NH—C(O)—CH═CH—, and R¹⁹ is

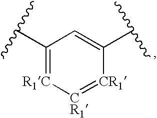

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

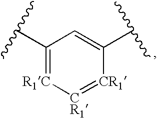

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

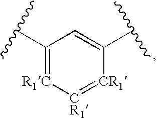

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

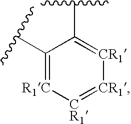

R³¹ is not 3,5-dinitrophenyl, 4-butoxyphenyl,

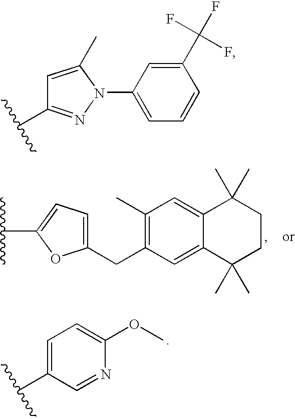

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In certain embodiments, when R²¹ is —NH—C(O)—, R¹⁹ is not

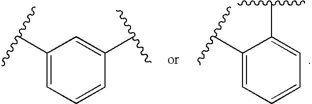

In certain embodiments, when R²¹ is —NH—C(O)—, R¹⁹ is not optionally substituted pyrazolyl, thiazolyl, thienyl, pyrrolyl or pyrimidinyl; when R²¹ is —NH—C(O)—CH2— or —NH—C(O)—NH—, R¹⁹ is not pyrazolyl; and/or when R²¹ is —NH—, R¹⁹ is not optionally substituted pyridyl, thiazolyl, pyrazolyl, thiadiazolyl, or oxadiazolyl.

In a more particular aspect, the invention provides [symbol] -modulating compounds of Structural Formula (XXVII):

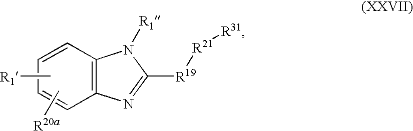

or a salt thereof, wherein:
each R²⁰ and R^{20a} is independently selected from H or a solubilizing group;

each R₁′ and R₁″ is independently selected from H or optionally substituted C₁-C₃ straight or branched alkyl;

R¹⁹ is selected from:

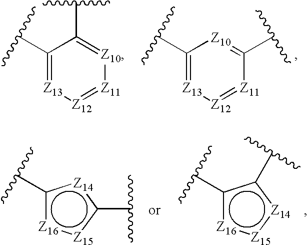

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
one to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—C(O)—CH₂—, and R¹⁹ is pyrazolyl, R³¹ is not unsubstituted indolyl or unsubstituted phenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;
when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;
when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and
when R²¹ is —NH—C(O)— and R¹⁹ is thiazolyl or pyrimidinyl, R³¹ is not unsubstituted phenyl.

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.
In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In yet another aspect, the invention provides compounds of Structural Formula (XXVII):

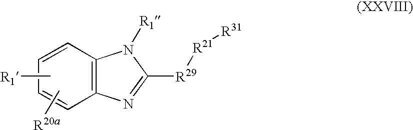

or a salt thereof, wherein:
each R²⁰ and R^{20a} is independently selected from H or a solubilizing group;
each R₁′ and R₁″ is independently selected from H or optionally substituted C₁-C₃ straight or branched alkyl;
R²⁹ is selected from:

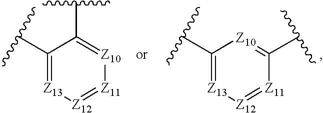

wherein:
Z₁₀, Z₁₁, Z₁₂ and Z₁₃ is independently selected from N, CR²⁰, or CR₁′, wherein one of Z₁₀, Z₁₁, Z₁₂ or Z₁₃ is N; and
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl.

In certain embodiments, R³¹ is optionally substituted phenyl, such as 3-methoxyphenyl, 3,4-dimethoxyphenyl, 3,4,5-trimethoxyphenyl, or 4-dimethylaminophenyl.

In certain embodiments, R²¹ is —NH—C(O)—.

In preferred embodiments, R²¹ is —NH—C(O)— and R³¹ is an optionally substituted phenyl, such as 3-methoxyphenyl, 3,4-dimethoxyphenyl, 3,4,5-trimethoxyphenyl, or 4-dimethylaminophenyl.

In a further aspect, the invention provides novel: [symbol] -modulating compounds of Formula (VI):

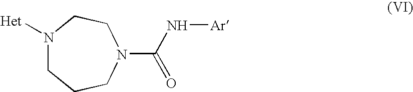

or a salt thereof, wherein:

Het is an optionally substituted heterocyclic aryl group; and

Ar′ is an optionally substituted carbocyclic or heterocyclic aryl group.

In certain embodiments, Het comprises one N heteroatom and 1 to 2 additional heteroatoms independently selected from N, O or S, such as oxazolopyridyl.

In certain embodiments, Ar′ is selected from optionally substituted phenyl, benzothiazolyl, or benzoxazolyl. When Ar′ is substituted phenyl, typically it is substituted with 1 to 3 substituents independently selected from halo, methyl, O-methyl, S-methyl or N(CH₃)₂, morpholino, or 3,4 dioxymethylene.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

In the compounds described above, bivalent groups disclosed as possible values for variables can have either orientation, provided that such orientation results in a stable molecule. Preferably, however, the left hand side of a bivalent group (e.g., —NR₁′—C(O)—) is attached to a bivalent arylene or heteroarylene group (e.g., R¹⁹) and the right hand side of a bivalent group is attached to a monovalent aryl group (e.g., R³¹).
[symbol] -modulating compounds of the invention having hydroxyl substituents, unless otherwise indicated, also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

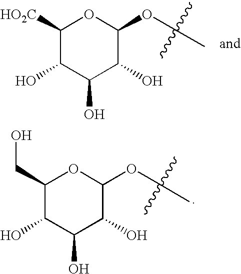

TABLE 4

| COM- | | | ED₅₀ | ED₅₀ | FOLD |
|---|---|---|---|---|---|
| POUND | | | FP | MS | ACT. |
| NO | [M + H]+ | STRUCTURE | ASSAY | ASSAY | MS |
| 1 | | 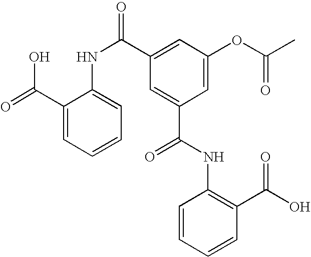 | A | NT | |
| 2 | | 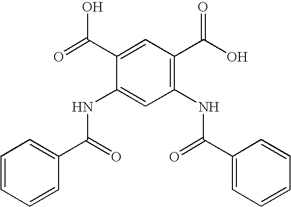 | D | NT | |
| 3 | | 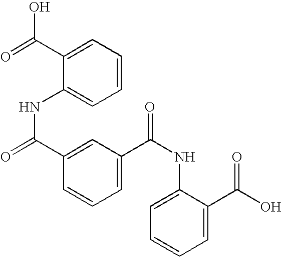 | B | NT | |
| 4 | | 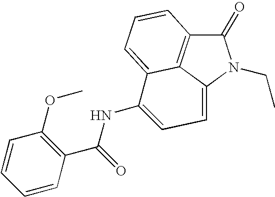 | N/A | NT | |
| 5 | | 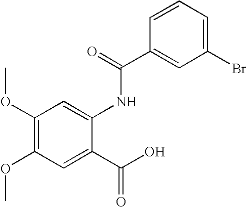 | B | NT | |
| 6 | | 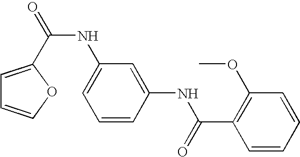 | D | NT | |
| 7 | 346 | 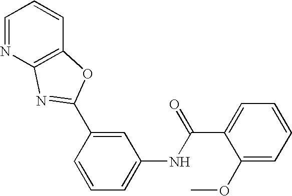 | A | NT | |
| 8 | | 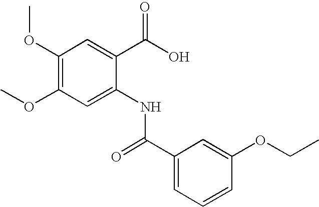 | B | NT | |
| 9 | | 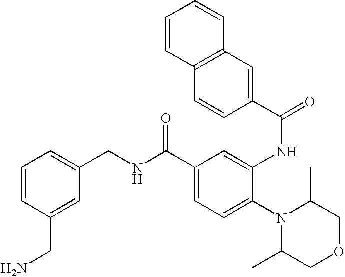 | C | NT | |
| 10 | | 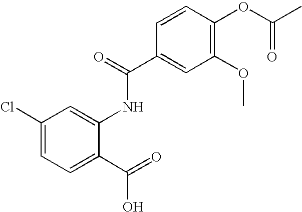 | D | NT | |
| 19 | 409.6 | 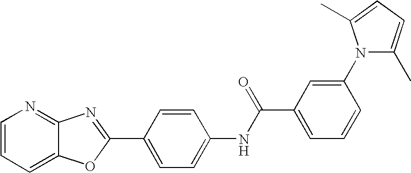 | D | D | C |
| 20 | 401.3 | 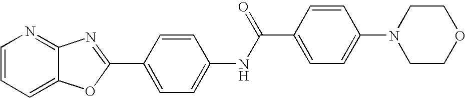 | D | D | C |
| 21 | 399.1 | 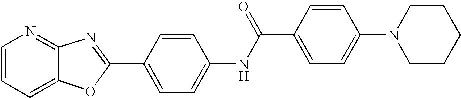 | D | D | C |
| 22 | 414.0 | 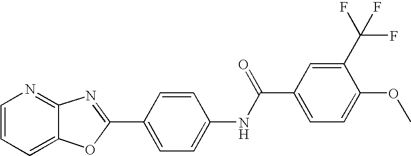 | D | D | C |
| 24 | 359.4 | 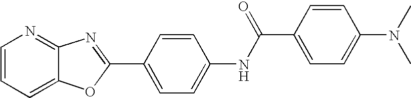 | D | D | C |
| 27 | 376.1 | 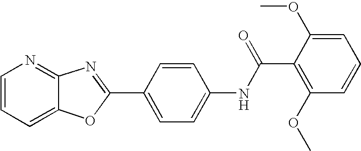 | D | D | C |
| 29 | 385.1 | 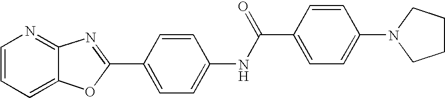 | D | D | C |
| 31 | 360.1 | 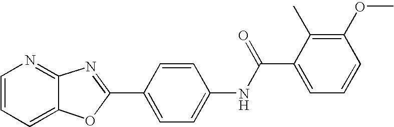 | D | D | C |
| 32 | 400.0 | 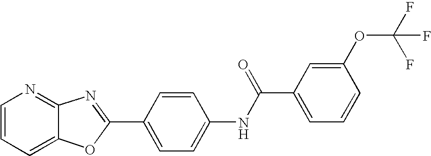 | D | D | C |
| 33 | 376.1 | 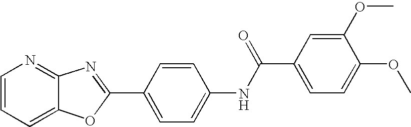 | D | D | C |
| 34 | 406.3 | 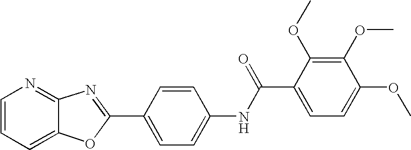 | D | D | C |
| 35 | 346.5 | 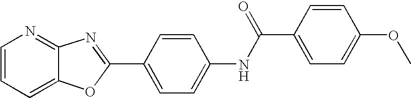 | D | D | C |
| 36 | 376.7 | 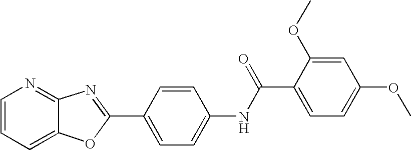 | D | D | C |
| 37 | 316.4 | 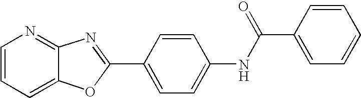 | D | D | C |
| 38 | 401.0 | 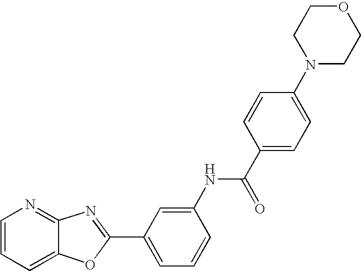 | D | D | C |
| 39 | 399.1 | 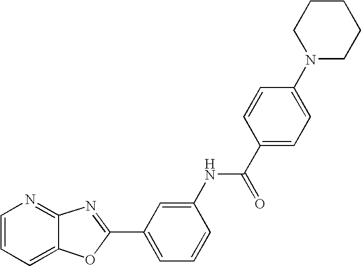 | D | D | C |
| 40 | 414.2 | 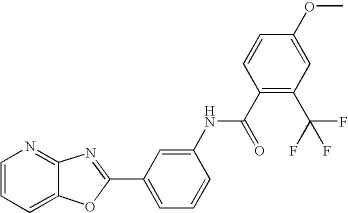 | D | D | C |
| 41 | 414.2 | 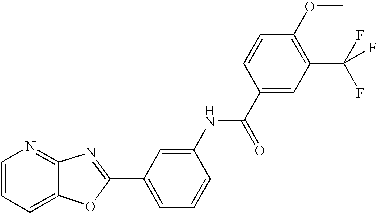 | D | D | C |
| 42 | 359.1 | 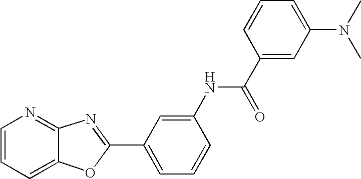 | A | A | B |
| 43 | 359.1 | 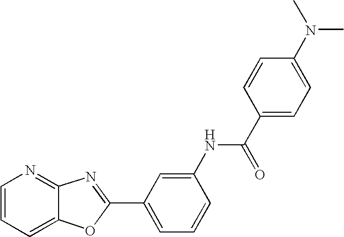 | A | A | B |
| 45 | 341.0 | 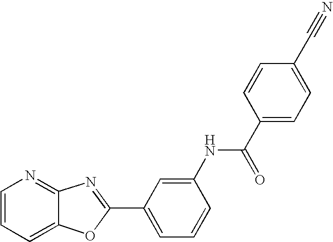 | D | D | C |
| 46 | 376.1 | 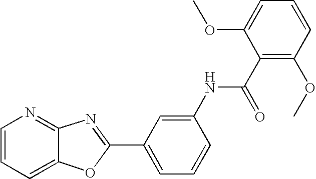 | D | D | C |
| 48 | 406.3 | 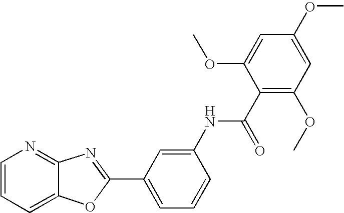 | D | D | C |
| 49 | 360.1 | 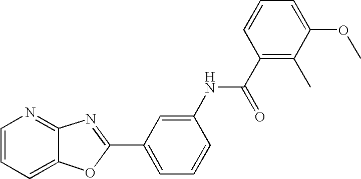 | D | A | B |
| 50 | 400.0 | 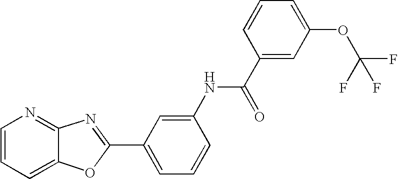 | NT | D | |
| 51 | 360.1 | 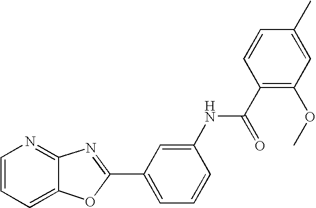 | A | A | B |
| 52 | 376.1 | 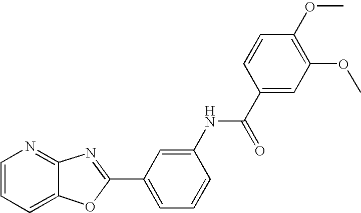 | A | A | B |
| 53 | 406.1 | 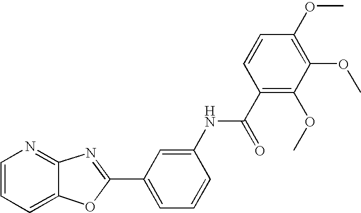 | D | D | C |
| 54 | 346.4 | 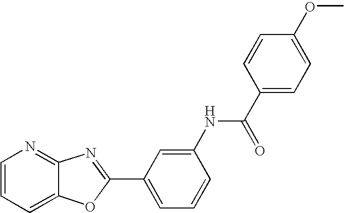 | NT | D | |
| 55 | 376.1 | 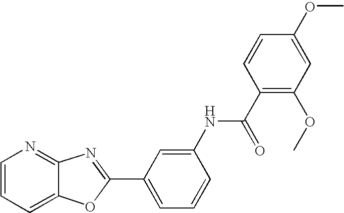 | A | A | B |
| 56 | 316.0 | 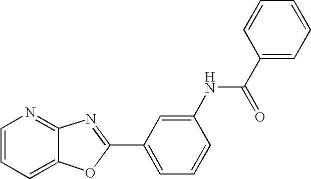 | D | A | B |
| 57 | 407.1 | 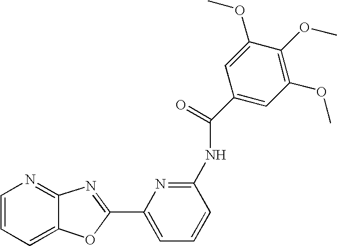 | | A | |
| 58 | 377.1 | 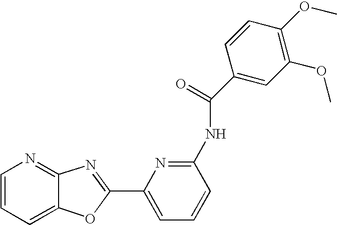 | | NA | |
| 59 | 318.1 | 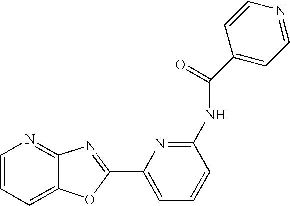 | | NA | |
| 60 | 413.1 | 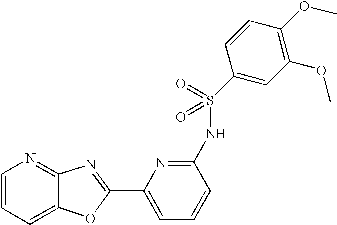 | | A | |
| 61 | 413.1 | 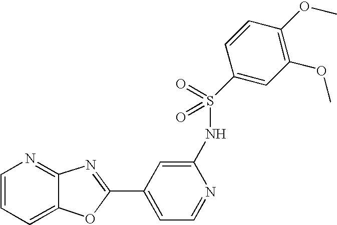 | | NA | |
| 62 | 349.0 | 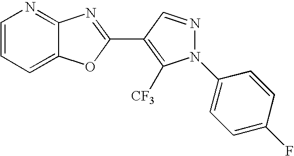 | | NA | |
| 63 | 377.1 | 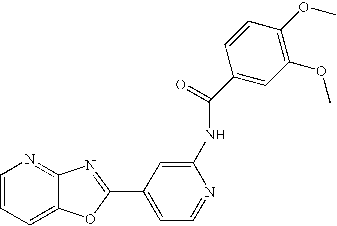 | | A | |
| 64 | 360.1 | 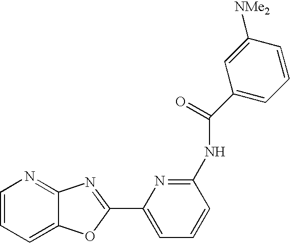 | | A | |
| 65 | 383.0 | 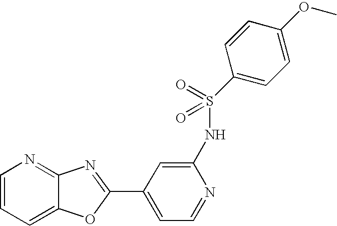 | | NA | |
| 66 | 407 | 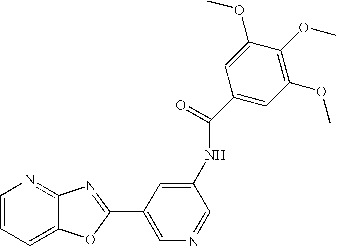 | | A | |
| 67 | 377 | 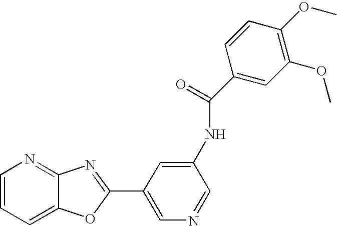 | | A | |
| 68 | 360 | 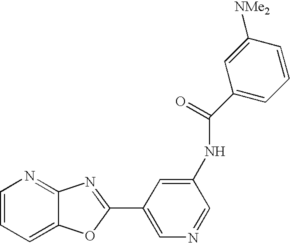 | | A | |
| 69 | 377.1 | 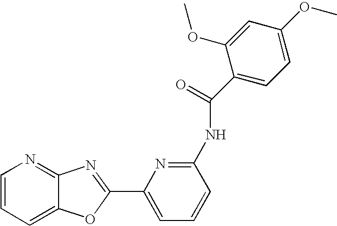 | | A | B |
| 70 | 360.1 | 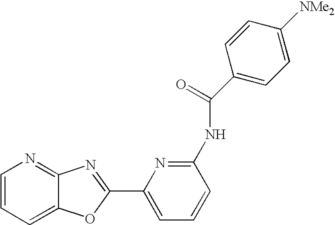 | | A | B |
| 71 | 376.1 | 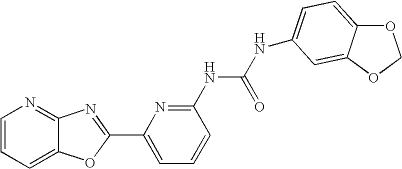 | | A | B |
| 72 | 422.1 | 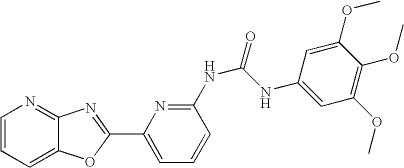 | | NT | |
| 73 | 377 | 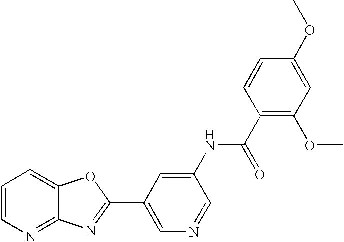 | | NT | |
| 74 | 412 | 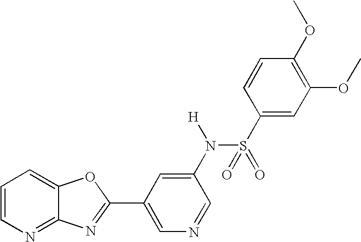 | | D | C |
| 75 | 407.1 | 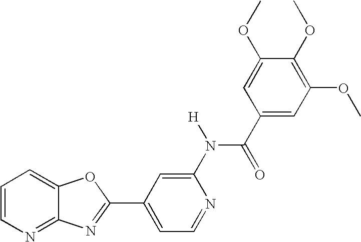 | | A | B |
| 76 | 360.1 | 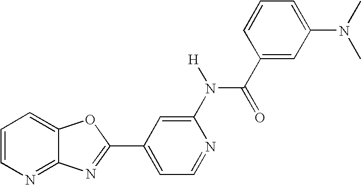 | | A | B |
| 77 | 376.1 | 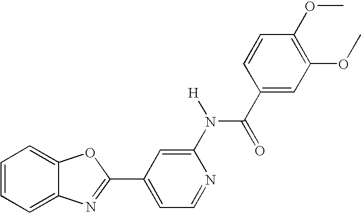 | | A | B |
| 78 | 445.1 | 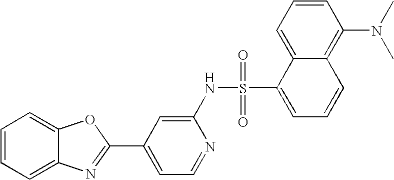 | | | |
| 79 | 338 | 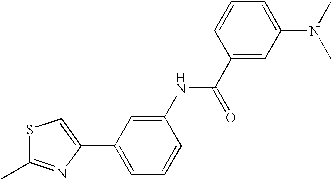 | | D | C |
| 80 | 355 | 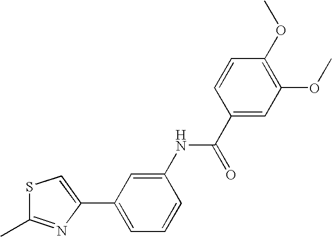 | | A | B |
| 81 | 354.5 | 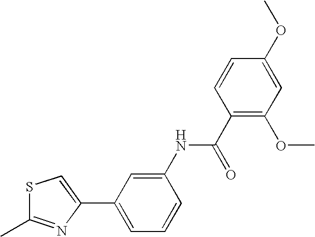 | | A | B |
| 82 | 402 | 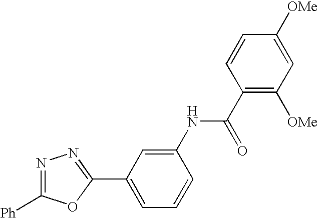 | | C | |
| 83 | 355 | 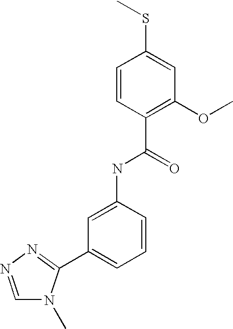 | | A | B |
| 84 | 417 | 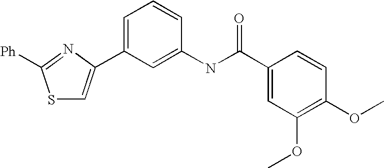 | | A | B |
| 85 | 335.1 | 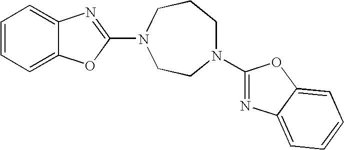 | | D | C |
| 86 | 375.1 | 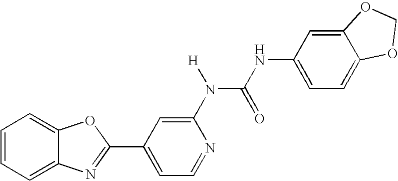 | | D | C |
| 87 | 375.1 | 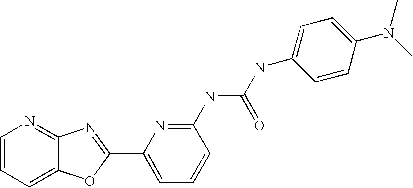 | | D | C |
| 88 | 382.1 | 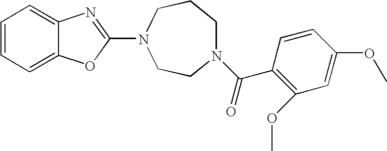 | | D | C |
| 89 | 365.1 | 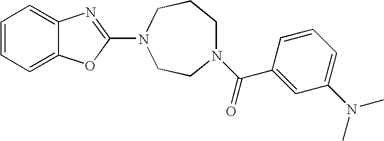 | | D | C |
| 90 | 381.1 | 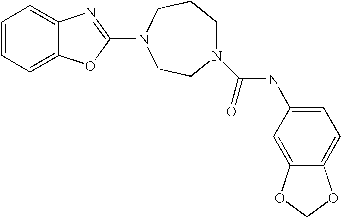 | | | |
| 91 | 412.1 | 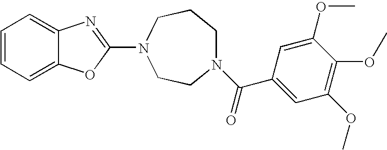 | | D | C |
| 92 | 308.1 | 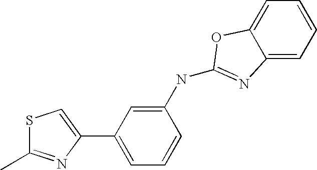 | | A | B |
| 93 | 329.1 | 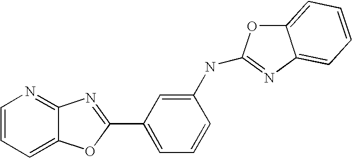 | | A | B |
| 94 | 434.1 | 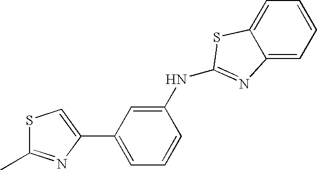 | | A | B |
| 95 | 345.1 | 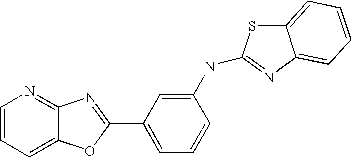 | | A′ | B |
| 96 | 376.1 | 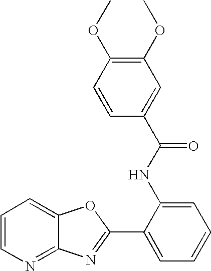 | | A′ | B |
| 97 | 359.1 | 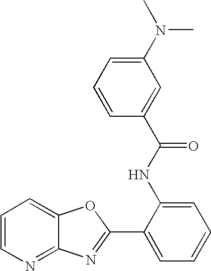 | | A′ | B |
| 98 | 408.1 | 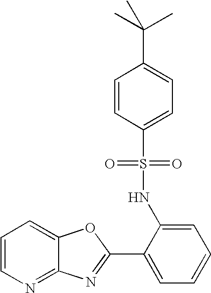 | | D | C |
| 99 | 391 | 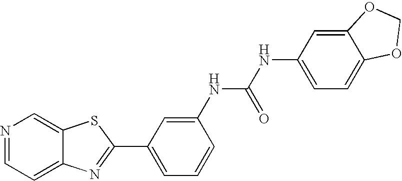 | | A′ | A |
| 100 | 376 | 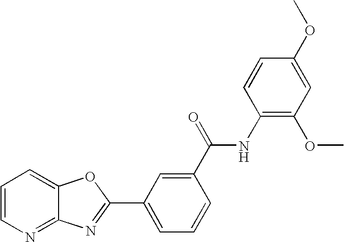 | | A | B |
| 101 | 376 | 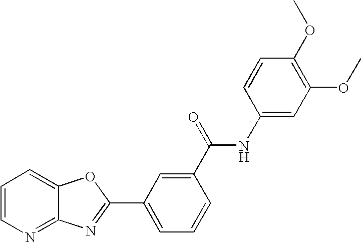 | | A | A |
| 102 | 406 | 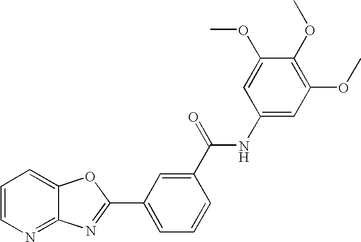 | | A | A |
| 103 | 374 | 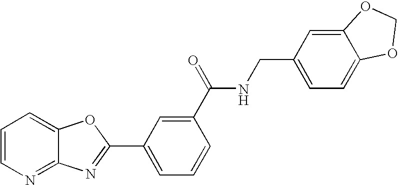 | | D | C |
| 104 | 346.1 | 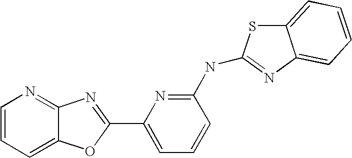 | | A′ | B |
| 105 | 330.1 | 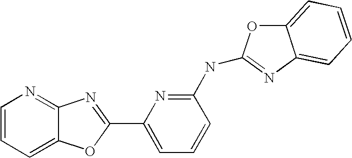 | | A′ | B |
| 106 | 406.1 | 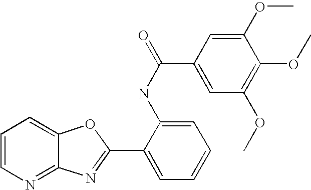 | | A′ | B |
| 107 | 488 | 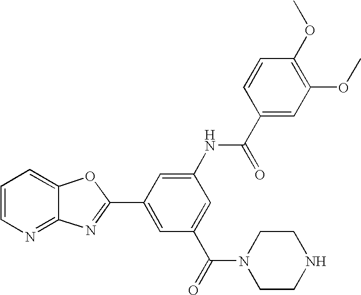 | | A | B |
| 108 | 324 | 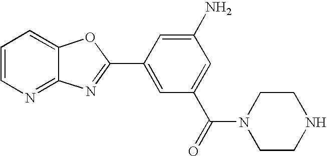 | | NA | |
| 109 | 401 | 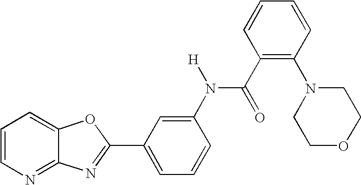 | | A | B |
| 110 | 381 | 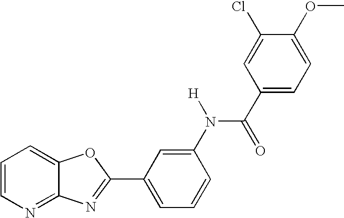 | | C | |
| 111 | 359 | 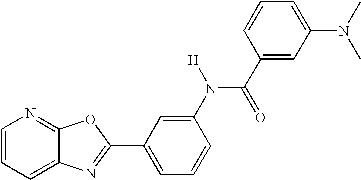 | | A | B |
| 112 | 376 | 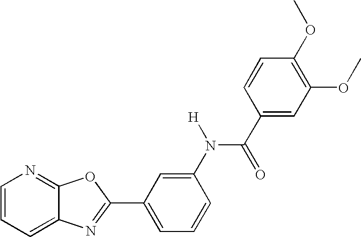 | | A | B |
| 113 | 375 | 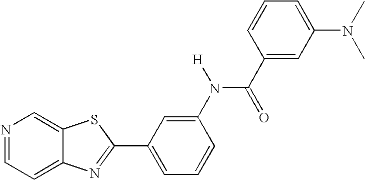 | | A | B |
| 114 | 392 | 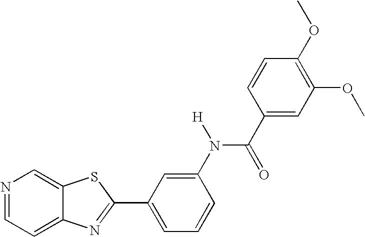 | | A′ | A |
| 115 | 422 | 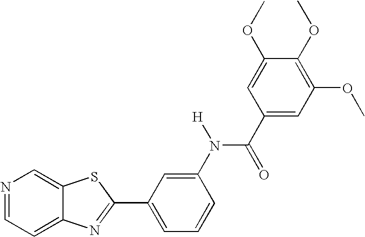 | | A | A |
| 116 | 386 | 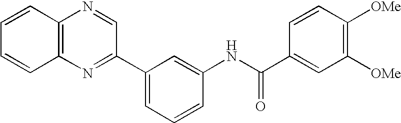 | | C | |
| 117 | 388 | 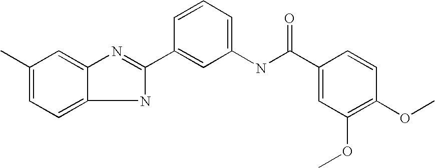 | | A | A |
| 118 | 410 | 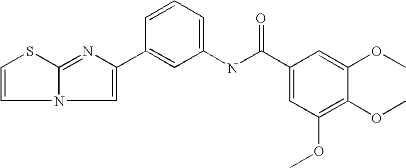 | | A | A |
| 119 | 375 | 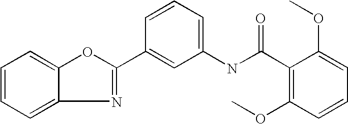 | | A | B |
| 120 | 391 | 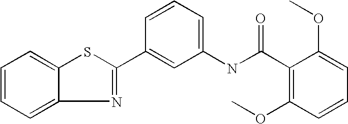 | | NT | |
| 121 | 414 | 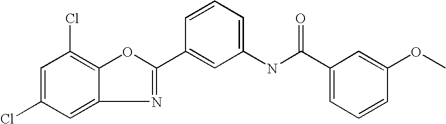 | | D | C |
| 122 | 417 | 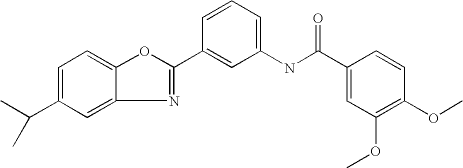 | | C | |
| 123 | 474 | 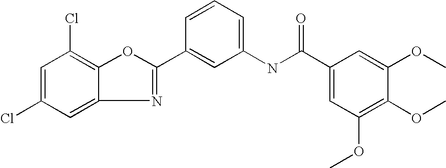 | | NT | |
| 124 | 391 | 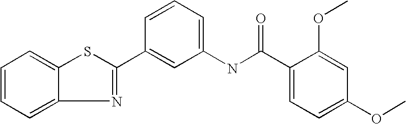 | | A | B |
| 125 | 433 | 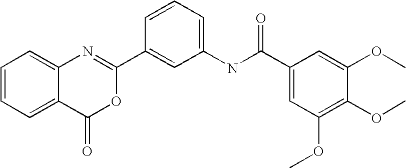 | | B | B |
| 126 | 374 | 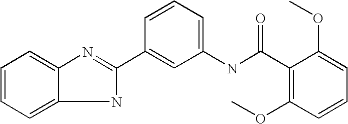 | | A | B |
| 127 | 355 | 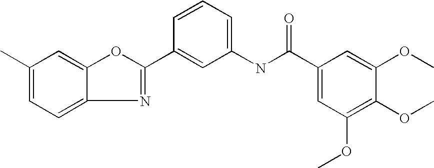 | | A | A |
| 128 | 388 | 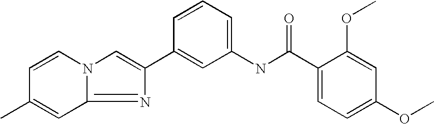 | | A | B |
| 129 | 418 | 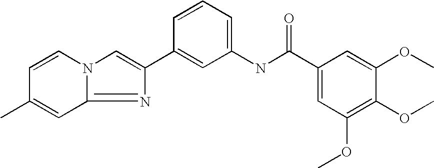 | | A′ | A |
| 130 | 358 | 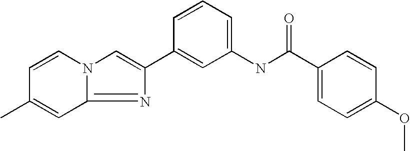 | | A | B |
| 131 | 418 | 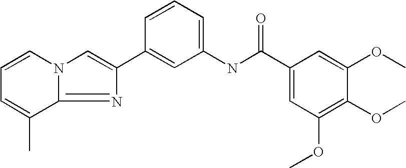 | | A | A |
| 132 | 350 | 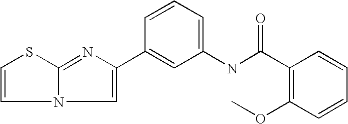 | | A | A |
| 133 | 480 | 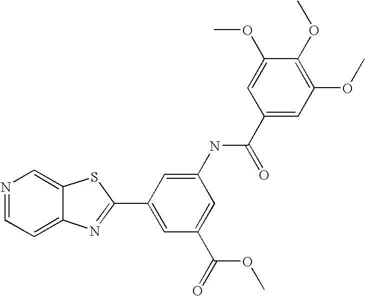 | | A′ | B |
| 134 | 466 | 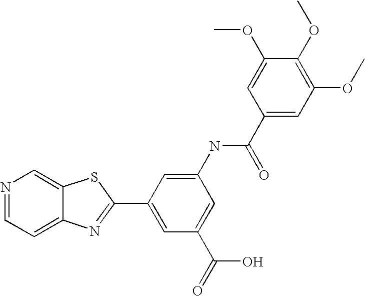 | | A′ | B |
| 135 | 452 | 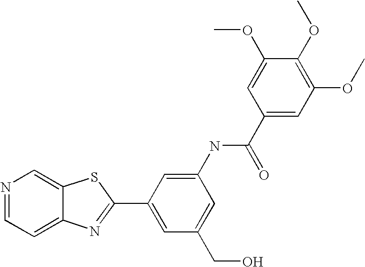 | | A′ | B |
| 136 | 434 | 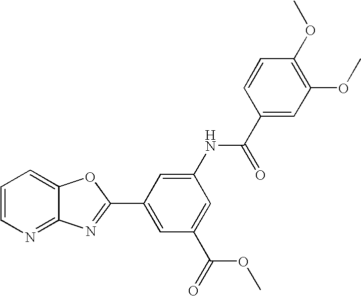 | | D | C |
| 137 | 420 | 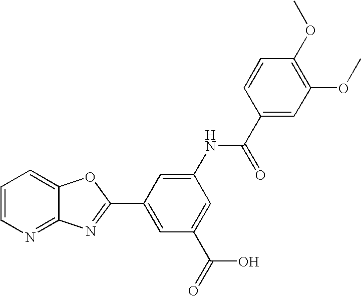 | | D | |
| 138 | 471 | 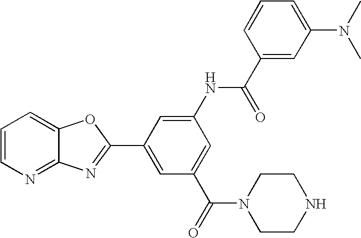 | | A | B |
| 139 | 488 | 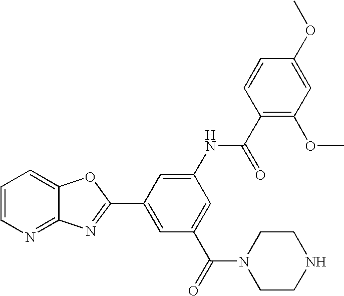 | | A | B |
| 141 | 374.1 | 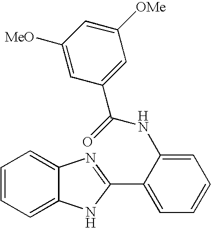 | | A′ | B |
| 142 | 374.1 | 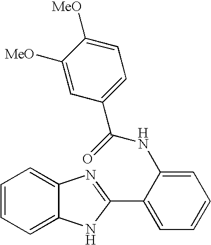 | | A′ | A |
| 143 | 410.1 | 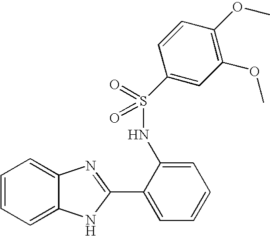 | | D | |
| 144 | 427.1 | 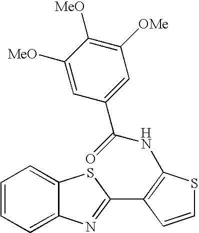 | | D | C |
| 145 | 397.1 | 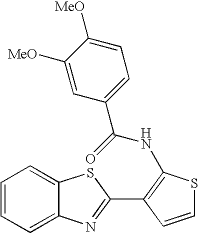 | | A′ | B |
| 146 | 397.1 | 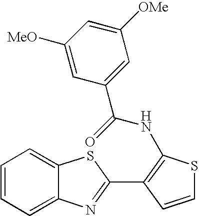 | | A′ | B |
| 147 | 392.1 | 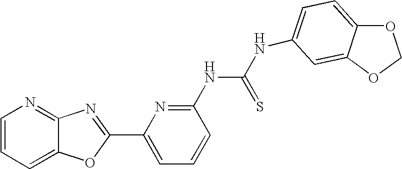 | | D | |
| 148 | 405.2 | 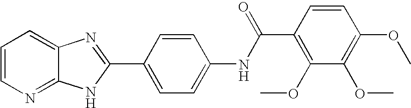 | | D | |
| 149 | 359.0 | 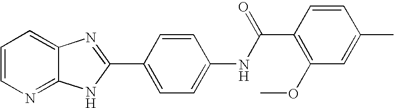 | | A′ | B |
| 150 | 375.0 | 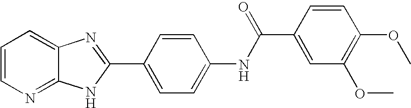 | | A′ | B |
| 151 | 375.0 | 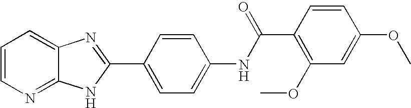 | | D | C |
| 152 | 392.1 | 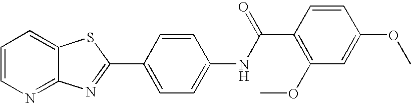 | | D | C |
| 153 | 490 | 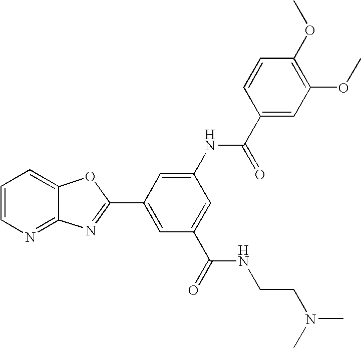 | | A | B |
| 154 | 473 | 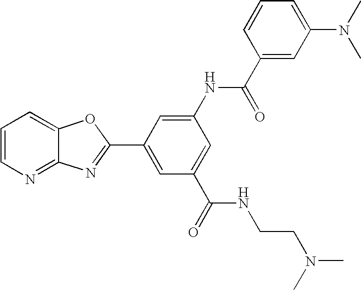 | | C | A |
| 155 | 490 | 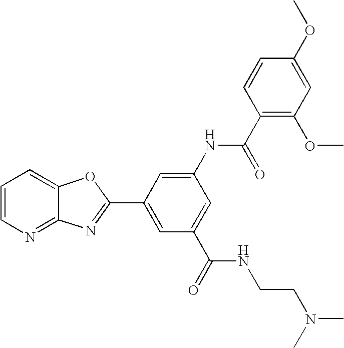 | | A | B |
| 156 | 433 | 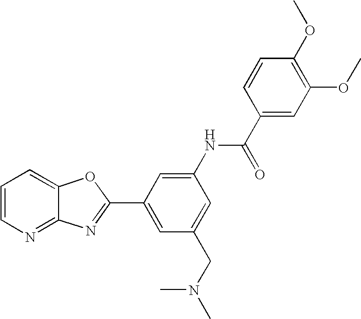 | | A | B |
| 157 | 416 | 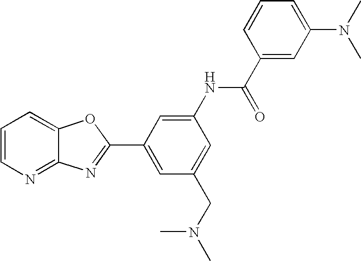 | | D | B |
| 158 | 433 | 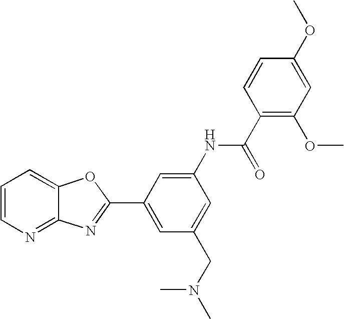 | | A | B |
| 159 | 474 | 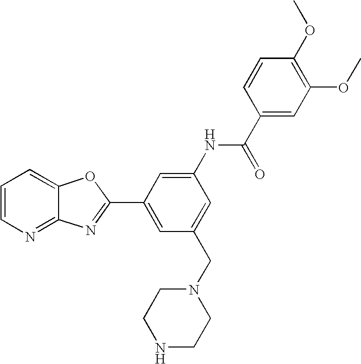 | | A | B |
| 160 | 457 | 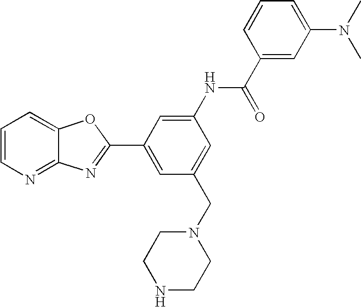 | | B | A |
| 161 | 474 | 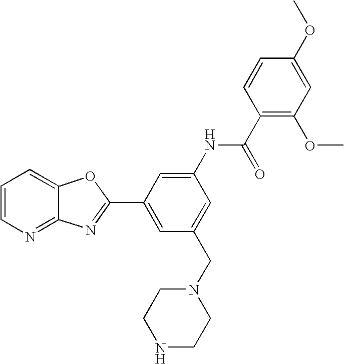 | | A | A |
| 162 | 392.1 | 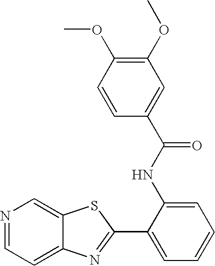 | | A′ | B |
| 163 | 422.1 | 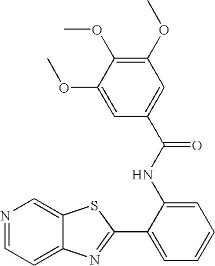 | | A′ | B |
| 164 | 488 | 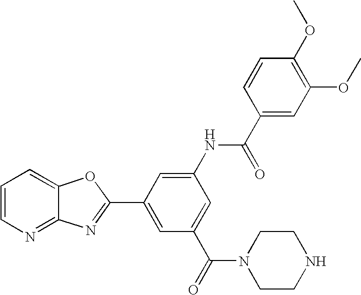 | | A | B |
| 165 | 416 | 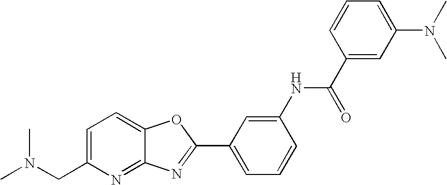 | | D | A |
| 166 | 457 | 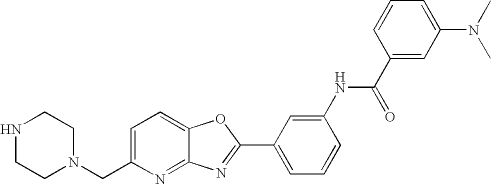 | | A | B |
| 167 | 373 | 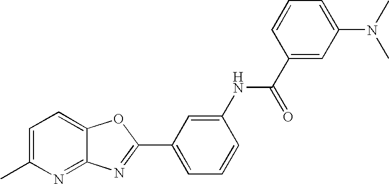 | | A | B |
| 168 | 388.1 | 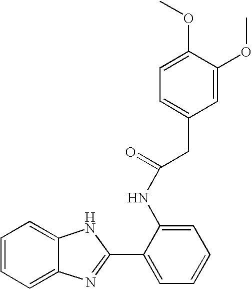 | | NA | C |
| 169 | 343.1 | 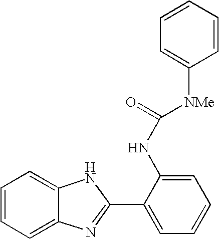 | | A′ | B |
| 174 | 479 | 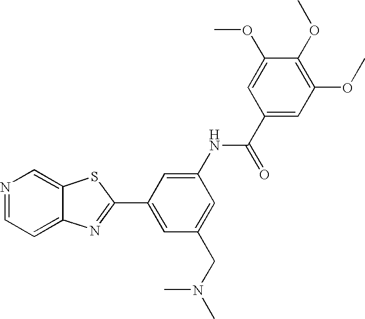 | | B | A |
| 175 | 323.1 | 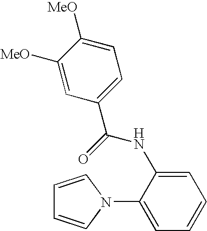 | | B | B |
| 176 | 354.1 | 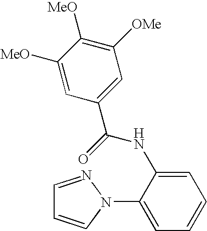 | | B | B |
| 177 | 324.1 | 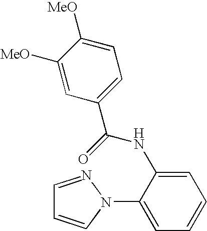 | | D | B |
| 178 | 437 | 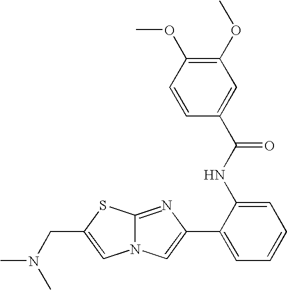 | | A | A |
| 179 | 467 | 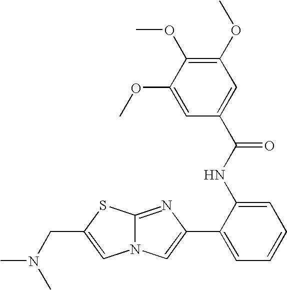 | | A′ | A |
| 180 | 358.0 | 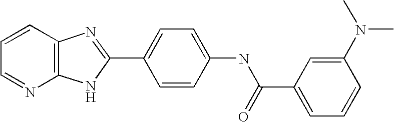 | | A | B |
| 181 | 405.0 | 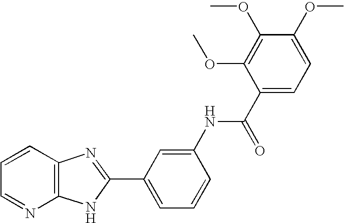 | | A′ | A |
| 182 | 359.0 | 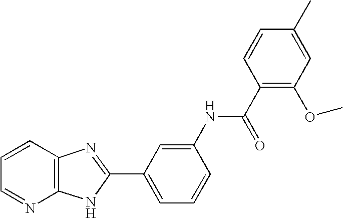 | | A′ | A |
| 183 | 358.0 | 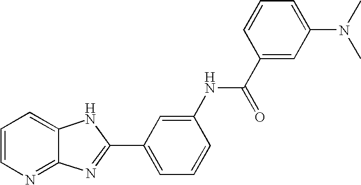 | | A | A |
| 184 | 375.0 | 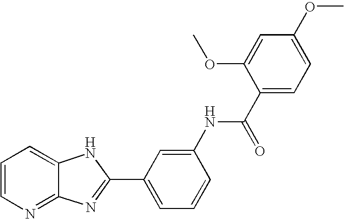 | | A′ | B |
| 185 | 374.9 | 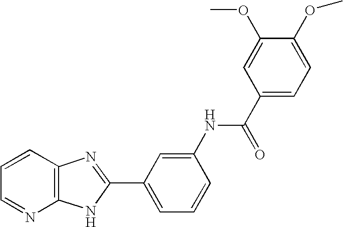 | | A′ | A |
| 186 | 405.3 | 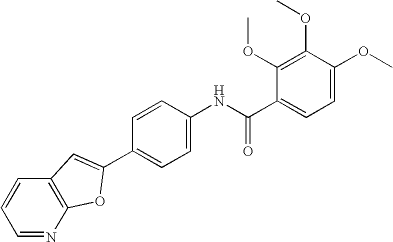 | | NA | C |
| 187 | 358.9 | 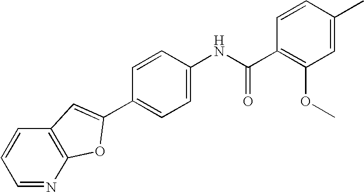 | | A | B |
| 188 | 358.9 | 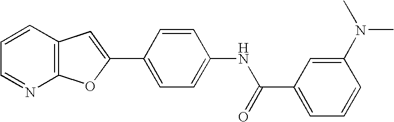 | | NA | C |
| 189 | 375.2 | 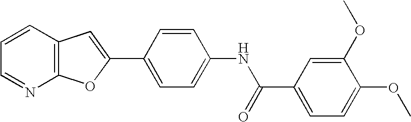 | | NA | C |
| 190 | 375.3 | 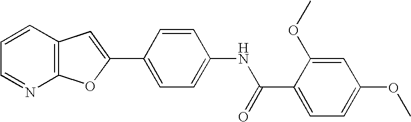 | | A′ | B |
| 191 | 375.0 | 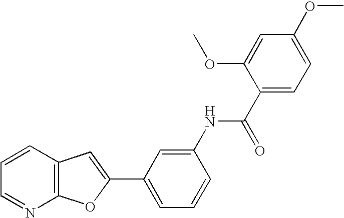 | | A′ | B |
| 192 | 375.0 | 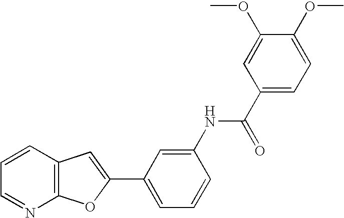 | | A | B |
| 193 | 358.0 | 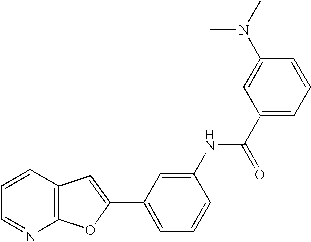 | | NA | C |
| 194 | 422.3 | 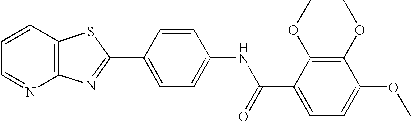 | | NA | C |
| 195 | 375.9 | 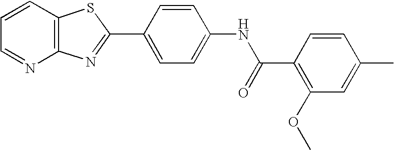 | | NA | C |
| 196 | 374.9 | 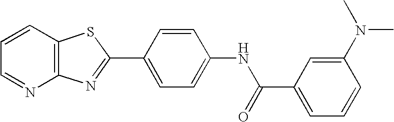 | | A | B |
| 197 | 391.1 | 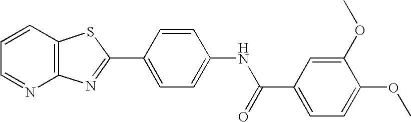 | | A′ | B |
| 198 | 422.4 | 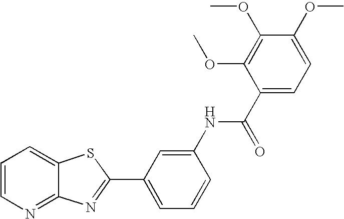 | | A | B |
| 199 | 375.9 | 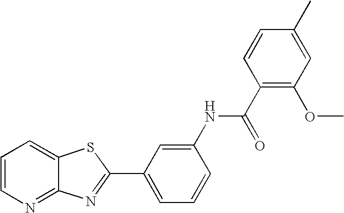 | | A′ | B |
| 200 | 375.4 | 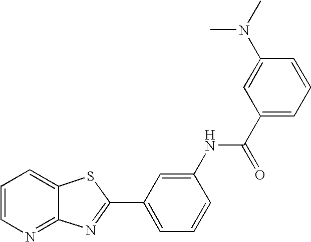 | | A′ | B |
| 201 | 391.9 | 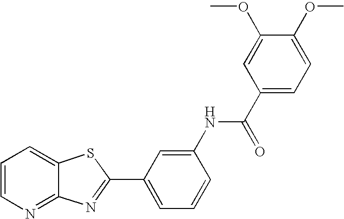 | | A | B |
| 202 | 392.4 | 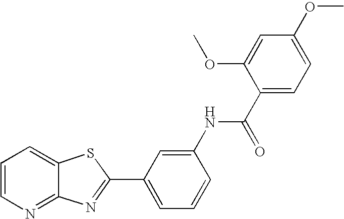 | | A′ | B |
| 203 | 380 | 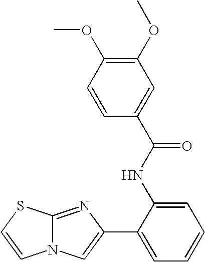 | | A′ | B |
| 204 | 410 | 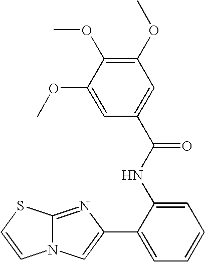 | | A′ | A |
| 205 | 437 | 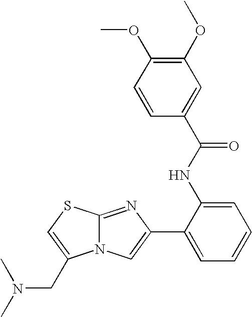 | | A | A |
| 206 | 467 | 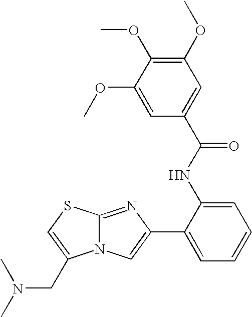 | | A | A |
| 207 | 478 | 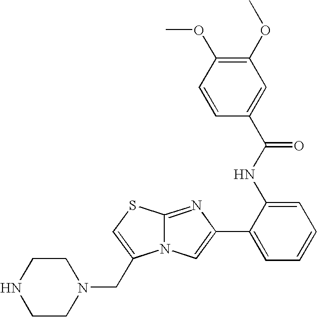 | | A | A |
| 208 | 508 | 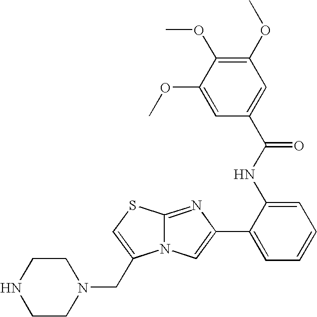 | | A | A |
| 209 | 479 | 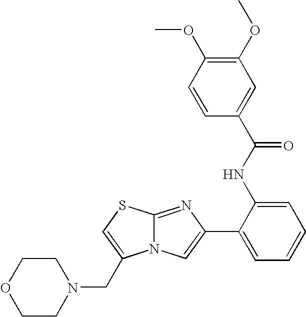 | | A | A |
| 210 | 509 | 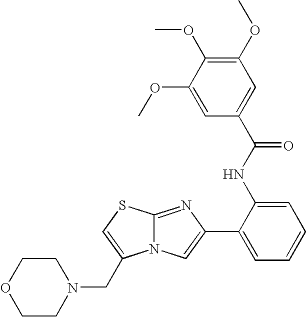 | | A | B |
| 211 | | 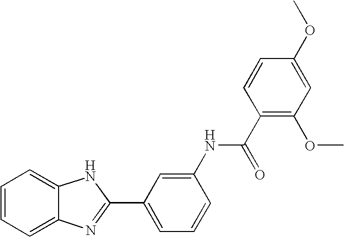 | | A′ | B |
| 212 | 362 | 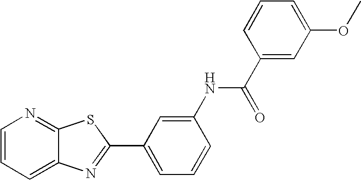 | | A | B |
| 213 | 392 | 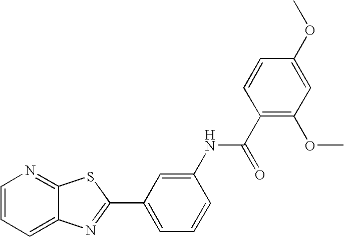 | | A′ | B |
| 214 | 392 | 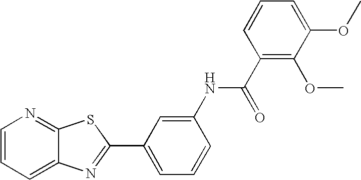 | | A | B |
| 215 | 392 | 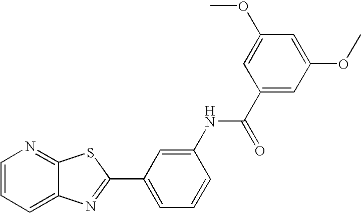 | | A′ | B |
| 216 | 362 | 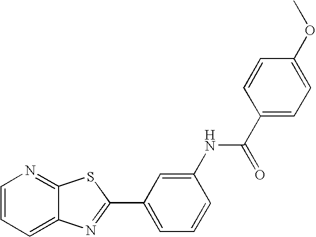 | | A′ | B |
| 217 | 422 | 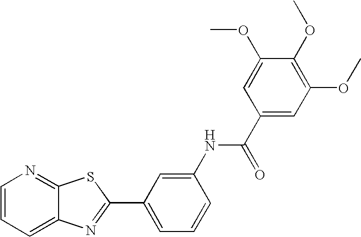 | | A′ | A |
| 218 | 405 | 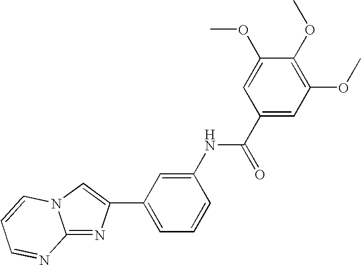 | | A′ | A |
| 219 | 419 | 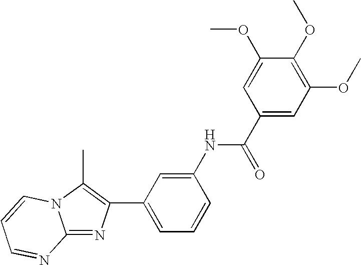 | | A | A |
| 220 | 423 | 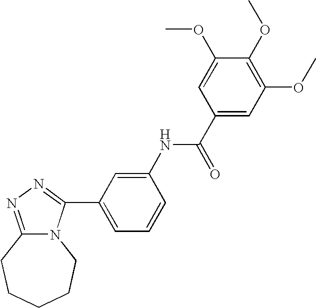 | | NA | C |
| 221 | 321.4 | 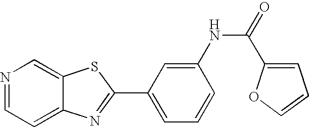 | | A | B |
| 222 | 366.8 | 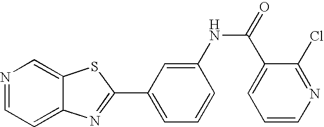 | | A | B |
| 223 | 337.4 | 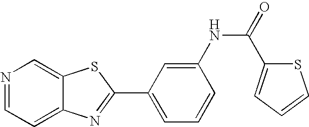 | | A′ | B |
| 225 | 332.4 | 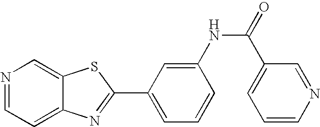 | | NA | C |
| 226 | 412.5 | 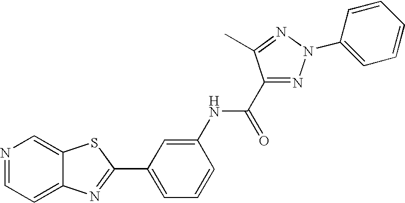 | | | |
| 227 | 333.4 | 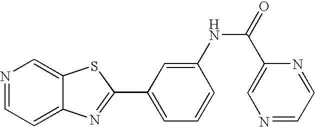 | | NA | C |
| 228 | 391.5 | 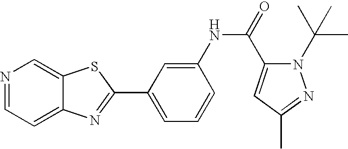 | | A | A |
| 229 | 418.5 | 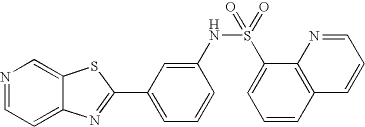 | | NA | C |
| 230 | 459.5 | 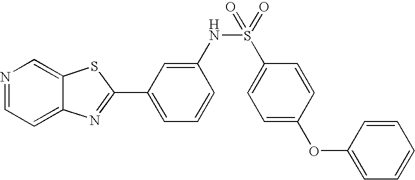 | | NA | C |
| 231 | 425.5 | 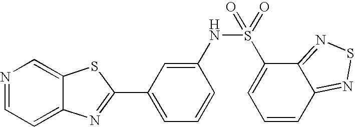 | | NA | C |
| 232 | 423.5 | 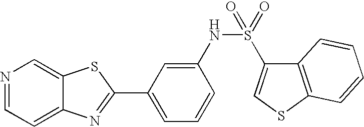 | | NA | C |
| 234 | 449.5 | 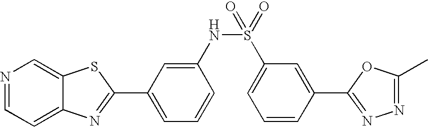 | | NA | C |
| 235 | 436.3 | 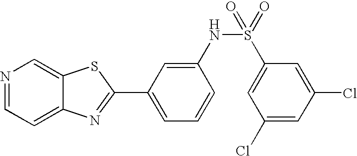 | | NA | C |
| 236 | 423.5 | 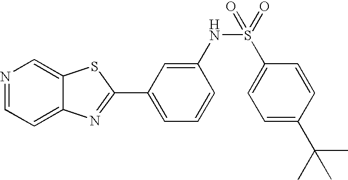 | | NA | C |
| 237 | 450.5 | 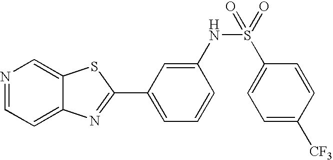 | | NA | C |
| 238 | 480.5 | 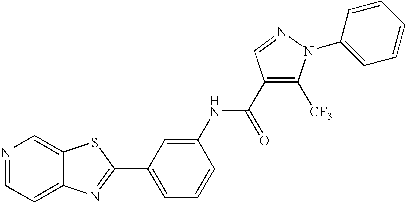 | | A′ | B |
| 239 | 466.5 | 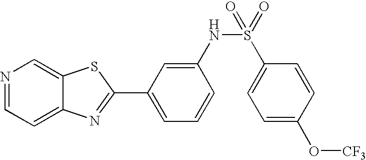 | | A | B |
| 240 | 392.4 | 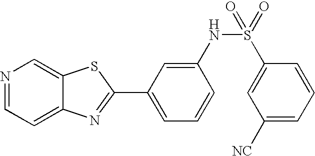 | | NA | C |
| 241 | 397.2 | 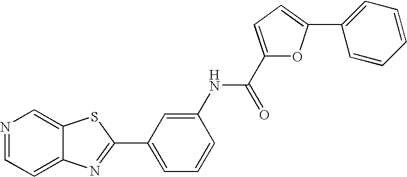 | | A′ | B |
| 244 | 332.4 | 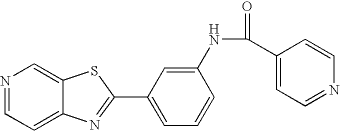 | | NA | C |
| 245 | 423.5 | 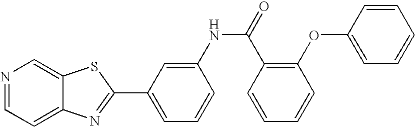 | | A′ | B |
| 246 | 391.5 | 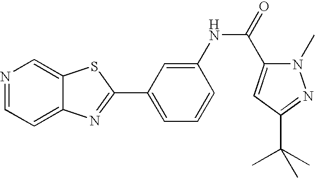 | | A′ | B |
| 247 | 413.5 | 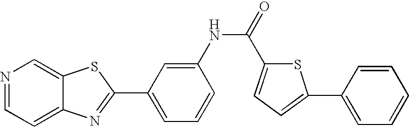 | | NA | C |
| 248 | 467.4 | 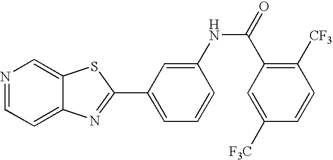 | | A | B |
| 249 | 395.9 | 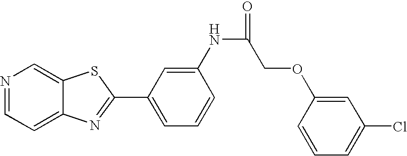 | | NA | C |
| 250 | 385.5 | 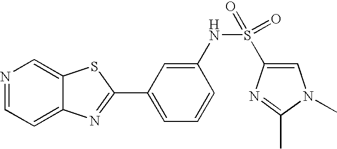 | | NA | C |
| 251 | 425.5 | 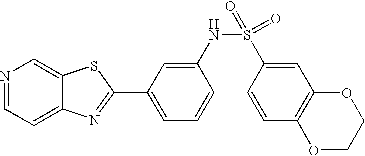 | | NA | C |
| 252 | 453.5 | 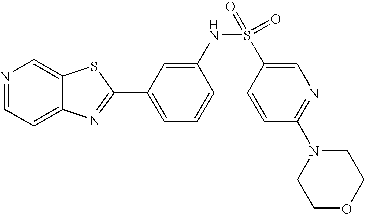 | | NA | C |
| 253 | 373.1 | 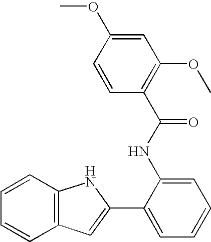 | | NA | C |
| 254 | 373 | 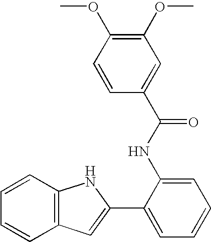 | | A′ | B |
| 255 | 403 | 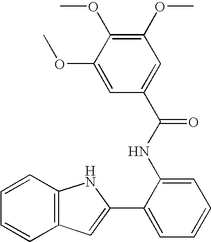 | | A′ | B |
| 256 | 356 | 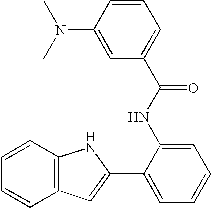 | | NA | C |
| 257 | 413.1 | 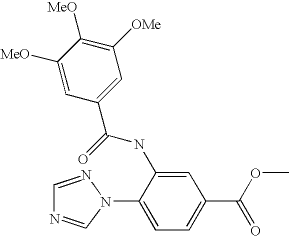 | | NA | C |
| 258 | 383.1 | 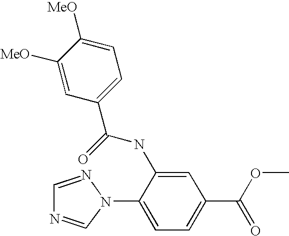 | | A | B |
| 259 | 405.1 | 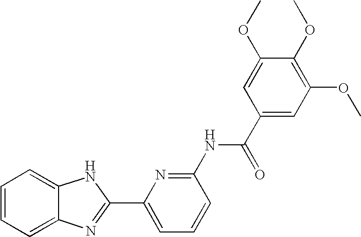 | | A′ | A |
| 260 | 375.1 | 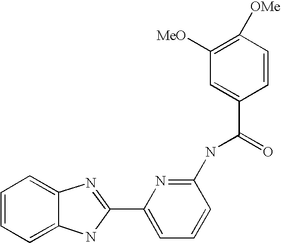 | | A′ | A |
| 261 | 375.1 | 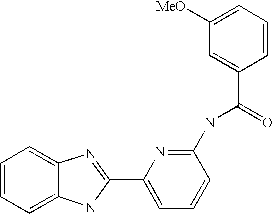 | | A′ | A |
| 262 | 374.1 | 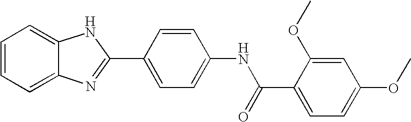 | | A′ | B |
| 263 | 404 | 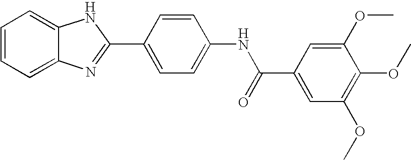 | | A | B |
| 264 | 357 | 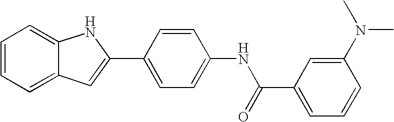 | | A′ | B |
| 265 | 374.1 | 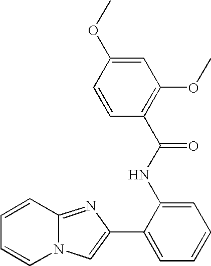 | | A | B |
| 266 | 374 | 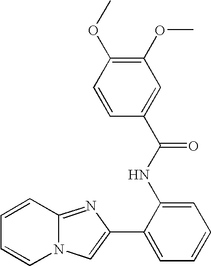 | | A′ | A |
| 267 | 404 | 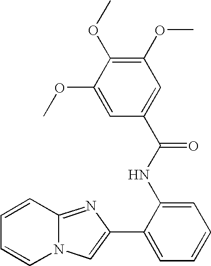 | | A′ | B |
| 268 | 357 | 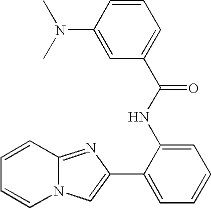 | | A′ | B |
| 270 | 478 | 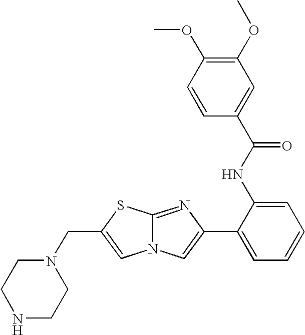 | | A | A |
| 271 | 508 | 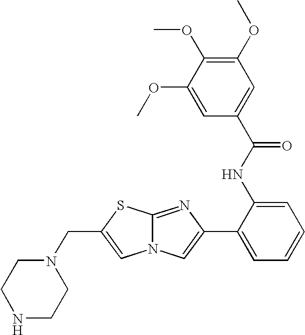 | | A′ | A |
| 272 | 392 | 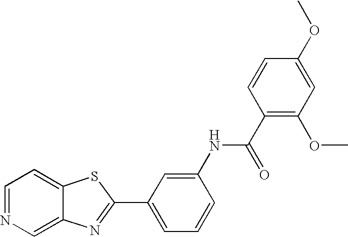 | | A′ | B |
| 273 | 422 | 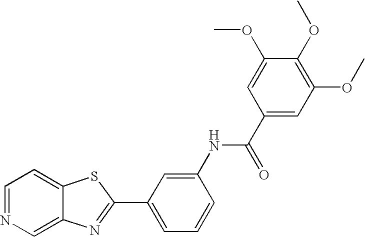 | | A | A |
| 276 | 375.1 | 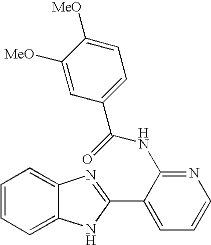 | | A′ | B |
| 280 | 381.5 | 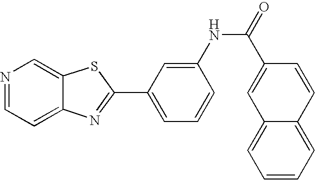 | | | |
| 282 | 386.5 | 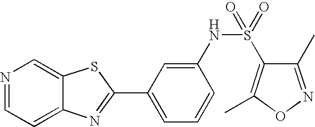 | | NA | C |
| 283 | 451.6 | 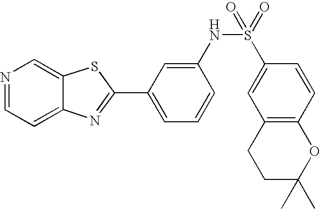 | | NA | C |
| 284 | 439.5 | 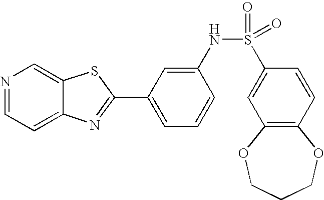 | | NA | C |
| 285 | 440.5 | 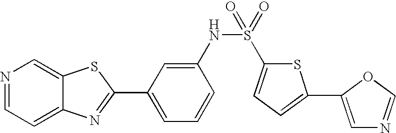 | | NA | C |
| 286 | 390.1 | 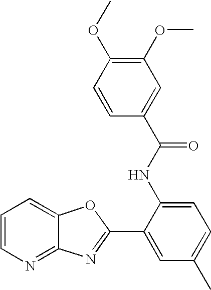 | | NA | C |
| 287 | 457.6 | 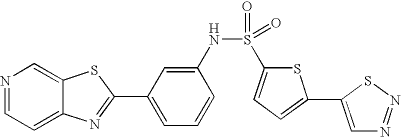 | | | |
| 288 | 424.5 | 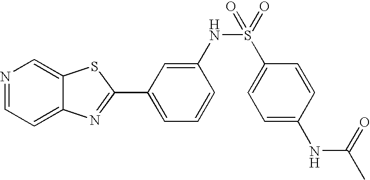 | | A | B |
| 289 | 427.5 | 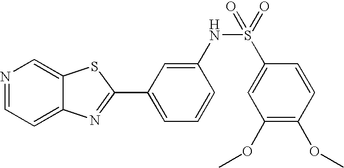 | | A | B |
| 290 | 445.5 | 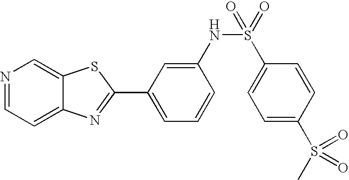 | | NA | C |
| 292 | 420.1 | 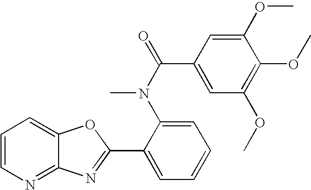 | | D | |
| 293 | 404.1 | 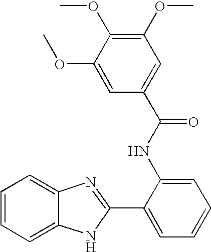 | | A′ | A |
| 294 | 374 | 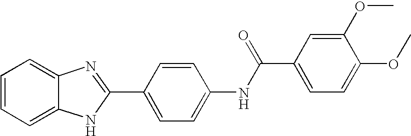 | | A′ | B |
| 295 | 252.1 | 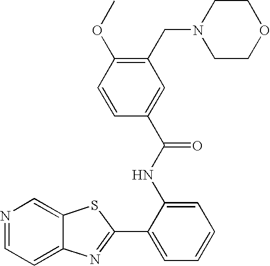 | | A′ | B |
| 296 | 376 | 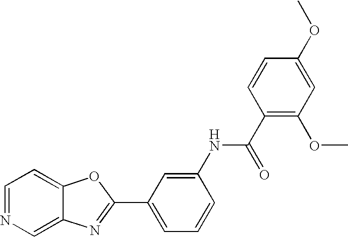 | | A | B |
| 297 | 376 | 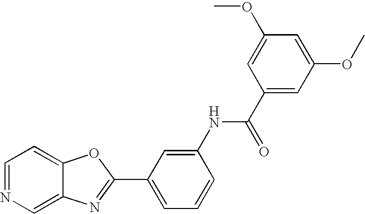 | | A | B |
| 298 | 406 | 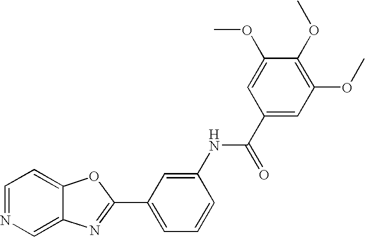 | | A | B |
| 299 | 359 | 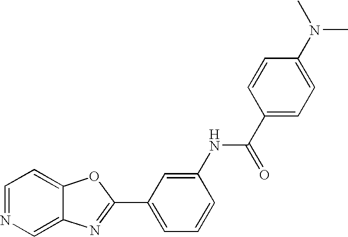 | | A | B |
| 303 | 437.5 | 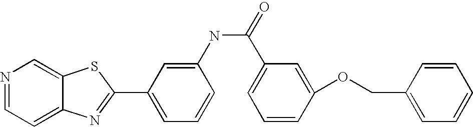 | | NA | |
| 304 | 336.4 | 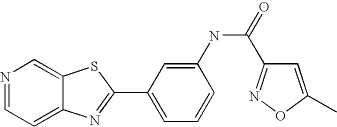 | | A′ | B |
| 305 | 414.5 | 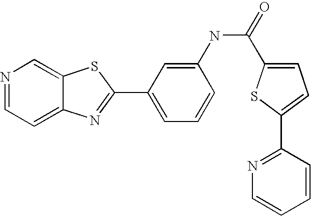 | | NA | |
| 306 | 424.5 | 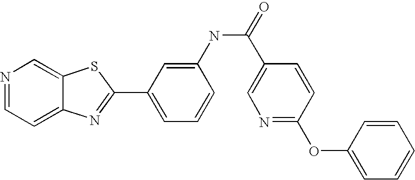 | | A | B |
| 307 | 382.4 | 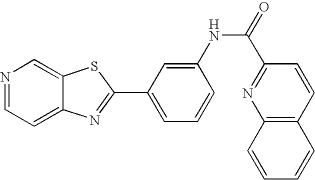 | | A′ | A |
| 308 | 400.3 | 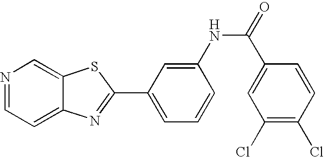 | | NA | |
| 309 | 367.4 | 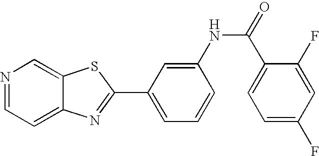 | | NA | |
| 310 | 387.5 | 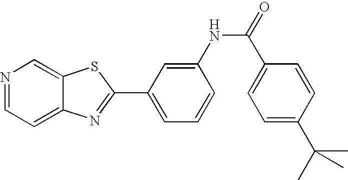 | | NA | |
| 311 | 359 | 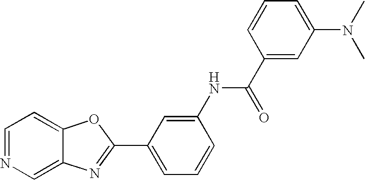 | | A | B |
| 313 | 418.1 | 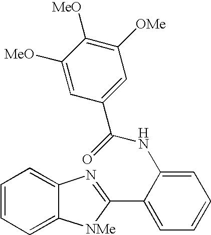 | | A | B |
| 314 | 388.1 | 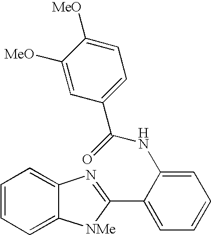 | | A | B |
| 315 | 388.1 | 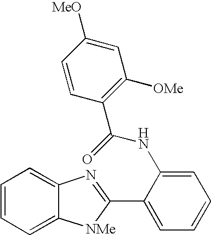 | | NA | |
| 317 | 392 | 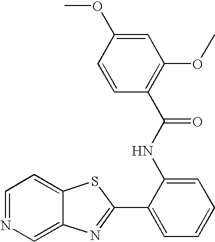 | | A′ | B |
| 318 | 392 | 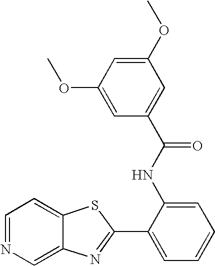 | | A | B |
| 319 | 422 | 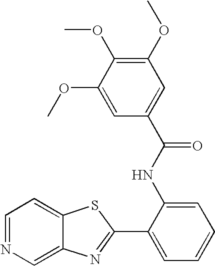 | | A′ | B |
| 320 | 375 | 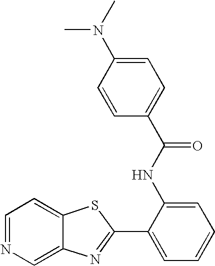 | | A′ | B |
| 321 | 375 | 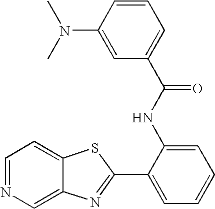 | | A′ | B |
| 322 | 392 | 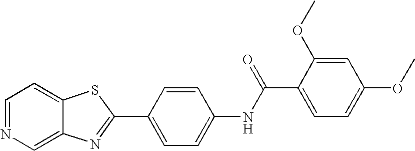 | | NA | |
| 323 | 392 | 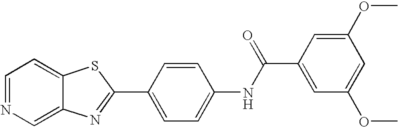 | | A | B |
| 324 | 422 | 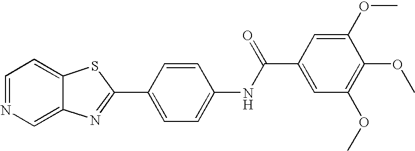 | | NA | |
| 325 | 375 | 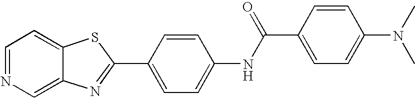 | | NA | |
| 326 | 478 | 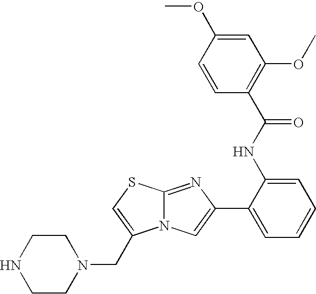 | | A | B |
| 327 | 461 | 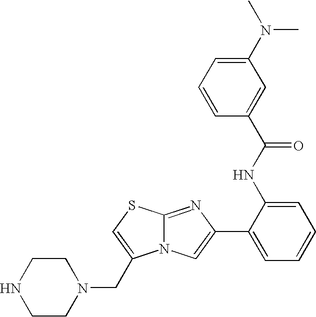 | | A | A |
| 328 | 418 | 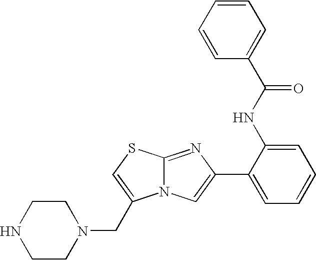 | | B | A |
| 329 | 462 | 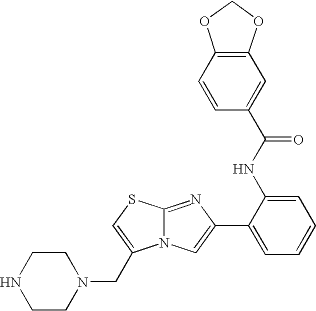 | | A | A |
| 330 | 443 | 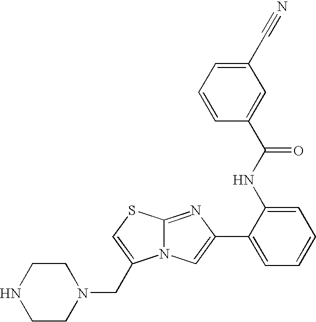 | | A | A |
| 331 | 335 | 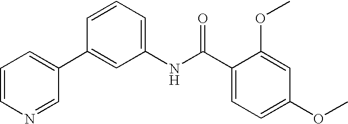 | | A | B |
| 332 | 335.1 | 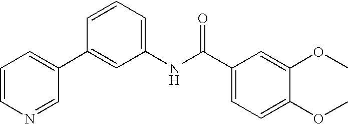 | | A | B |
| 333 | 365 | 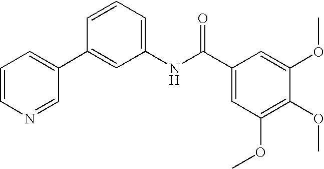 | | A | B |
| 334 | 340.1 | 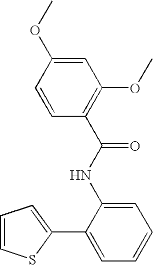 | | A | B |
| 335 | 340 | 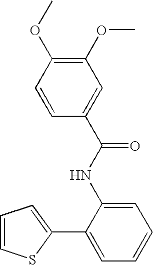 | | A | B |
| 336 10 | 370 | 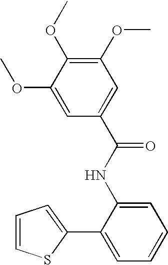 | | A | B |
| 337 | 443 | 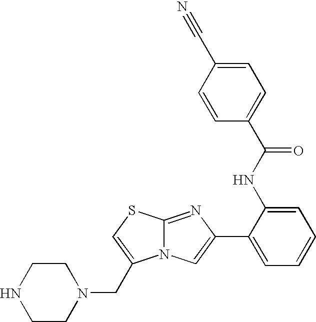 | | NA | |
| 338 | 458 | 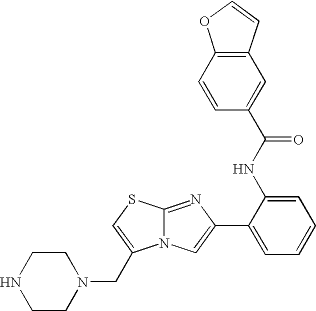 | | A | A |
| 339 | 376 | 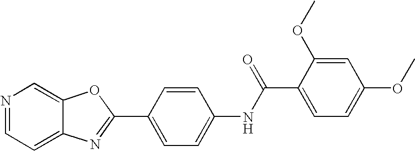 | | NA | |
| 340 | 376 | 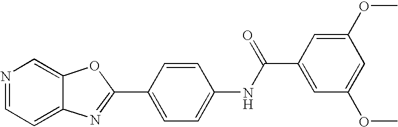 | | NA | |
| 341 | 406 | 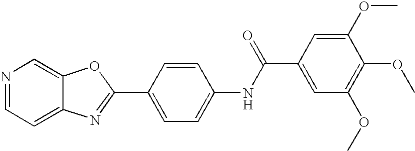 | | A′ | B |
| 342 | 359 | 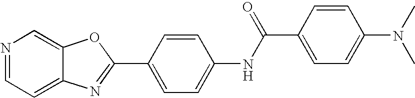 | | NA | |
| 343 | 406 | 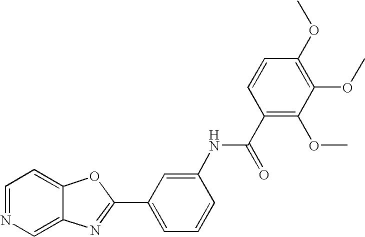 | | A | B |
| 344 | 375 | 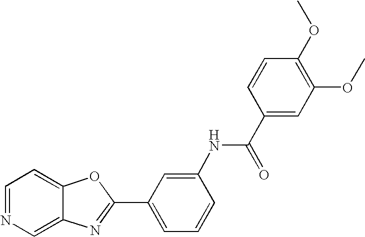 | | A′ | B |
| 345 | 376 | 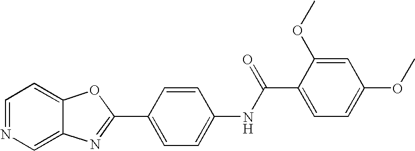 | | NA | |
| 346 | 376 | 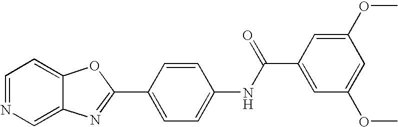 | | NA | |
| 347 | 406 | 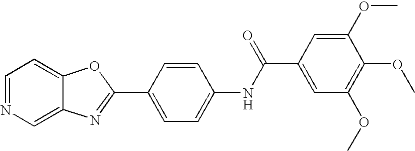 | | A′ | B |
| 348 | 359 | 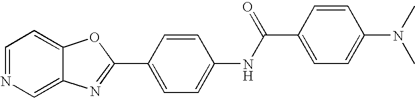 | | NA | |
| 349 | 375 | 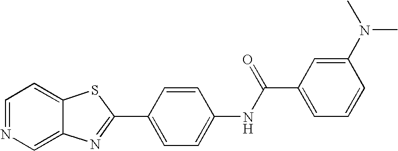 | | NA | |
| 350 | 431.1 | 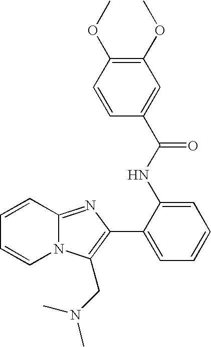 | | B | B |
| 351 | 461 | 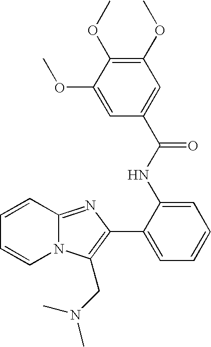 | | A | B |
| 359 | 472.1 | 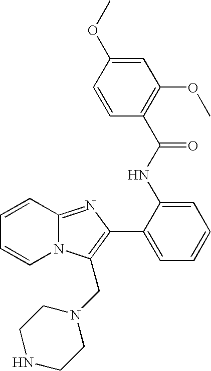 | | NA | |
| 362 | 502 | 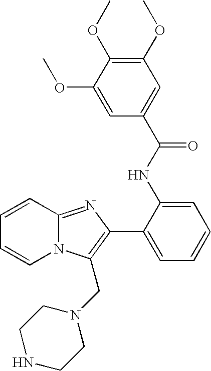 | | B | B |
| 364 | 472 | 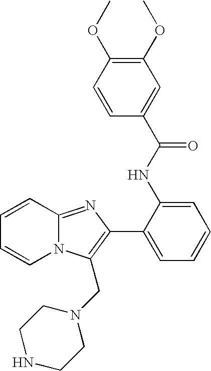 | | D | A |
| 367 | 359.1 | 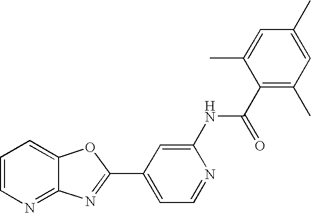 | | NA | |
| 369 | 350.8 | 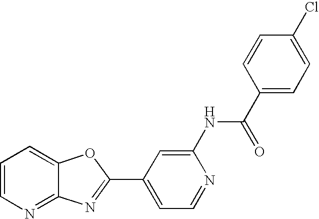 | | NA | |
| 370 | 437.0 | 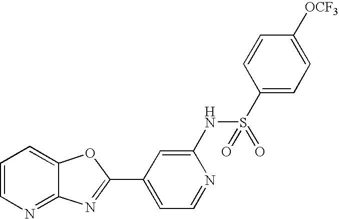 | | NA | |
| 371 | 381.1 | 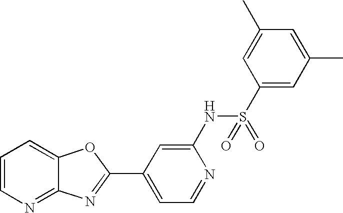 | | NA | |
| 372 | 431.1 | 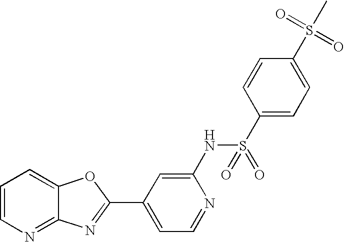 | | NA | |
| 373 | 445.0 | 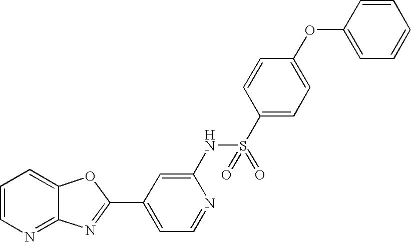 | | NA | |
| 374 | 421.1 | 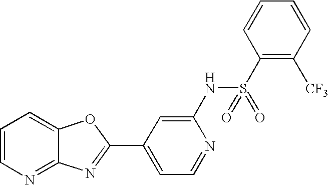 | | NA | |
| 375 | 358.2 | 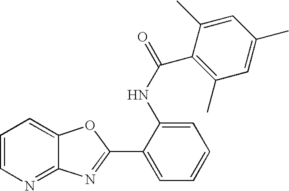 | | NA | |
| 376 | 350.0 | 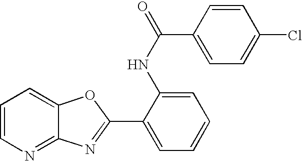 | | NA | |
| 377 | | 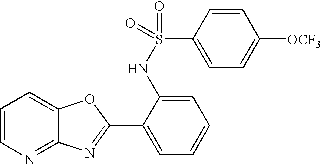 | | NA | |
| 378 | 412.1 | 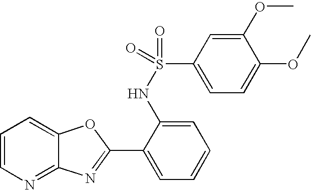 | | NA | |
| 379 | 380.0 | 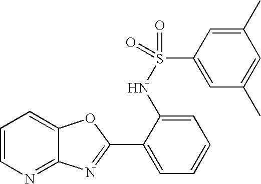 | | NA | |
| 380 | 429.9 | 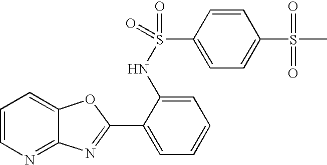 | | NA | |
| 381 | 444.1 | 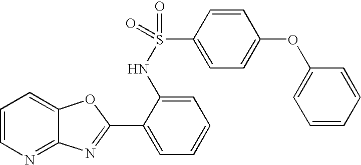 | | NA | |
| 382 | 420.0 | 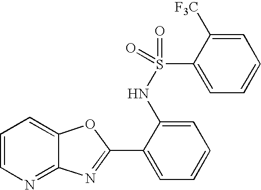 | | NA | |
| 383 | 515.7 | 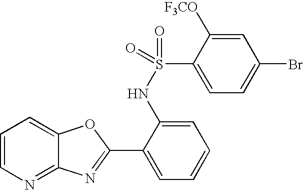 | | NA | |
| 384 | 487.8 | 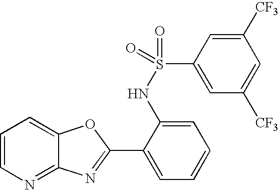 | | NA | |
| 385 | 397.2 | 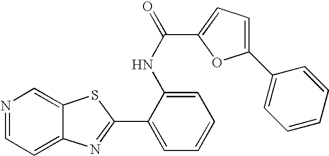 | | NA | |
| 387 | 366.8 | 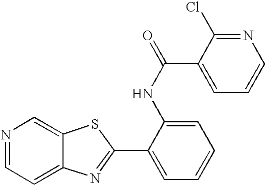 | | NA | |
| 390 | 412.5 | 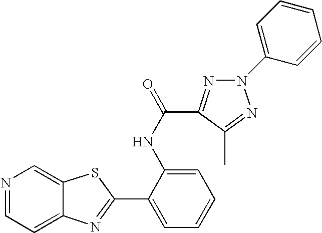 | | A | B |
| 391 | 333.4 | 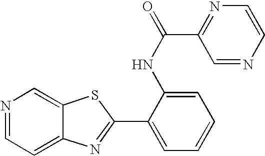 | | A | B |
| 392 | 424.5 | 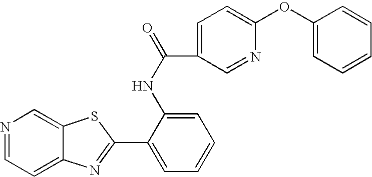 | | A | B |
| 393 | 400.3 | 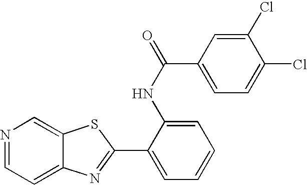 | | NA | |
| 394 | 457.6 | 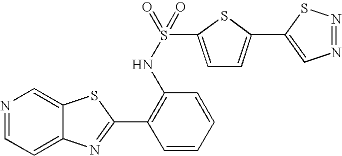 | | NA | |
| 396 | 389.5 | 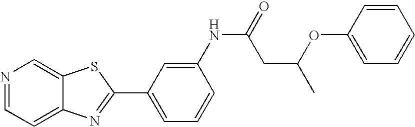 | | A | B |
| 398 | 460.1 | 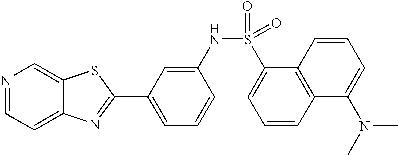 | | A | B |
| 399 | 424.5 | 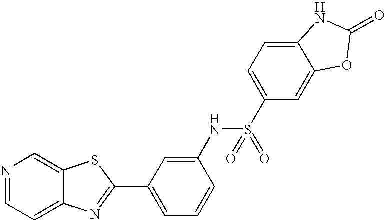 | | A | B |
| 400 | 392 | 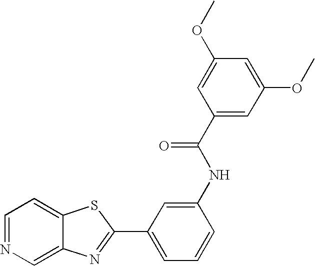 | | NA | |
| 401 | 422 | 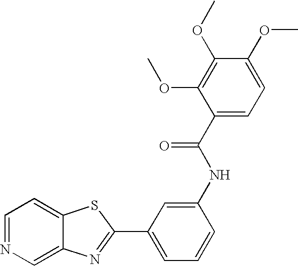 | | A | B |
| 402 | 375 | 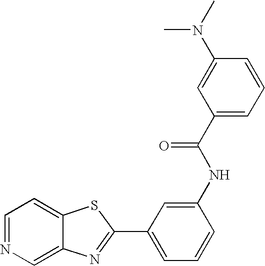 | | A′ | B |
| 403 | 375 | 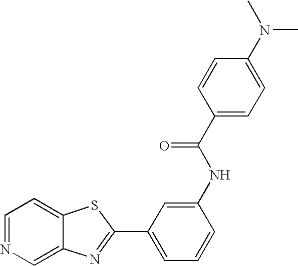 | | A′ | B |
| 404 | 376 | 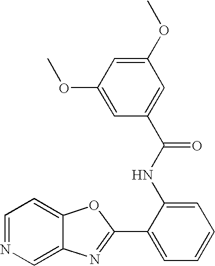 | | A′ | B |
| 405 | 406 | 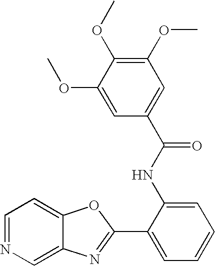 | | A′ | B |
| 406 | 359 | 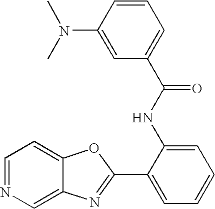 | | A′ | B |
| 407 | 359 | 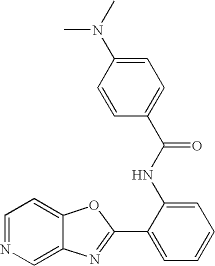 | | A′ | B |
| 408 | 359 | 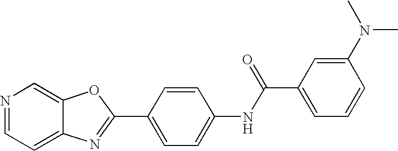 | | B | B |
| 409 | 422 | 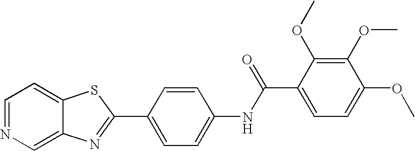 | | NA | |
| 410 | 359 | 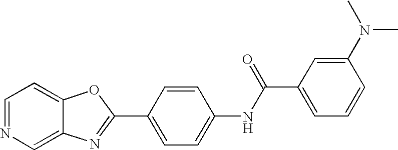 | | NA | |
| 411 | 422 | 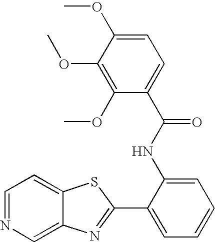 | | A | B |
| 412 | 406 | 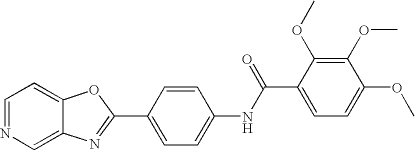 | | NA | |
| 413 | 385.1 | 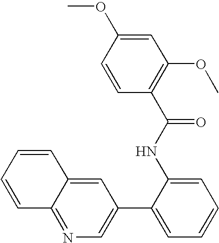 | | B | B |
| 414 | 385 | 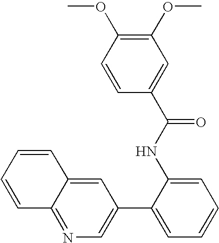 | | A | B |
| 415 | 415 | 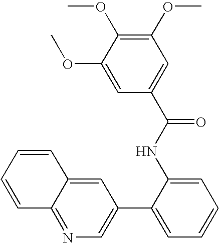 | | B | B |
| 416 | 368 | 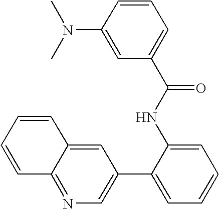 | | NA | |
| 419 | 406 | 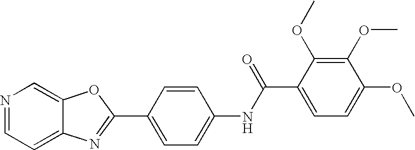 | | NA | |
| 420 | 376 | 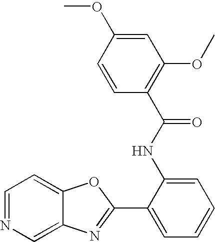 | | NA | |
| 421 | 406 | 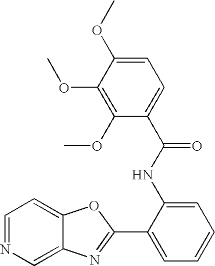 | | A | B |
| 422 | 382 | 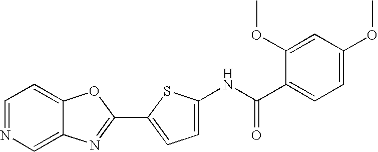 | | A | B |
| 423 | 382 | 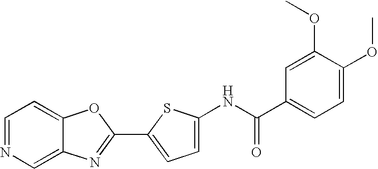 | | A′ | B |
| 424 | 382 | 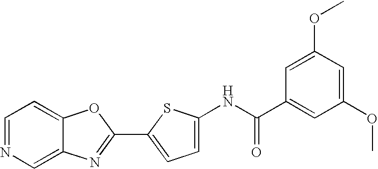 | | NA | |
| 425 | 412 | 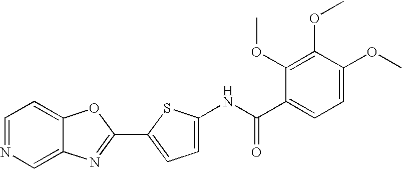 | | NA | |
| 426 | 412 | 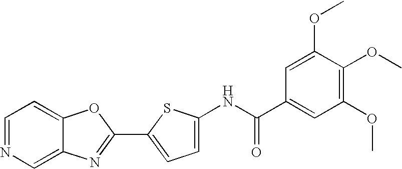 | | A′ | B |
| 427 | 365 | 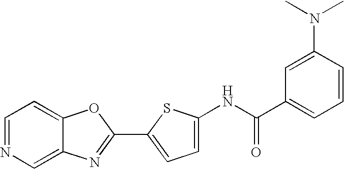 | | A′ | B |
| 428 | 365 | 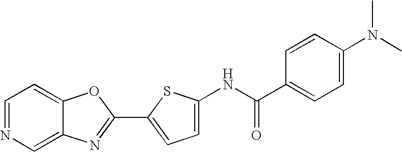 | | NA | |
| 429 | 376 | 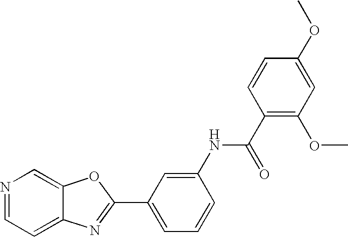 | | A′ | B |
| 430 | 406 | 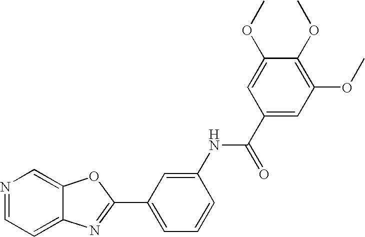 | | A | A |
| 431 | 359 | 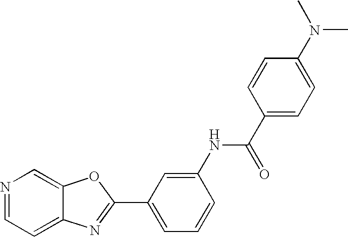 | | A | B |
| 436 | 445.1 | 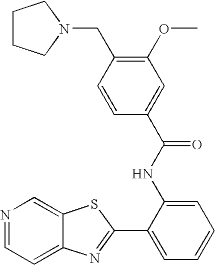 | | A | A |
| 437 | 375.1 | 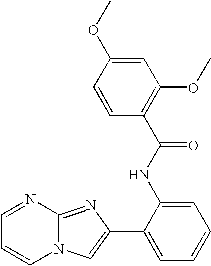 | | A | B |
| 438 | 375 | 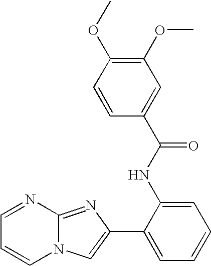 | | A′ | A |
| 439 | 405 | 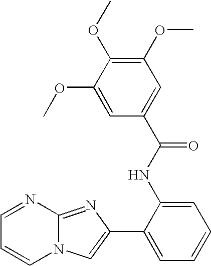 | | A′ | B |
| 440 | 468 | 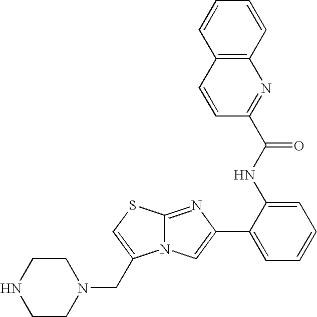 | | A′ | A |
| 441 | 470 | 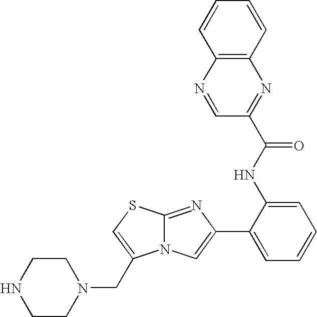 | | A′ | A |
| 442 | 472 | 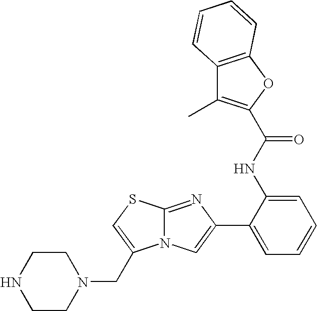 | | A′ | A |
| 443 | 436 | 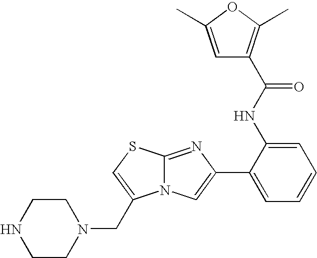 | | A | A |
| 444 | 464 | 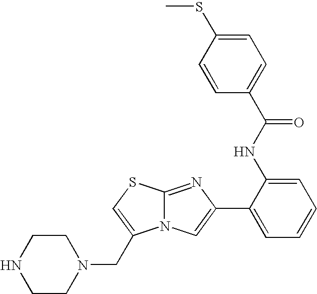 | | A | A |
| 445 | 432 | 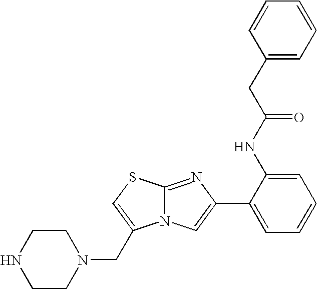 | | A | B |
| 446 | 424 | 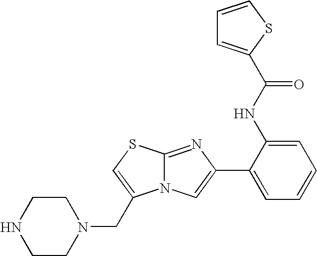 | | A | B |
| 447 | 484 | 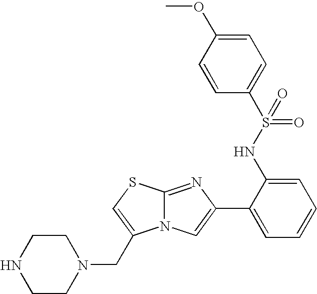 | | NA | |
| 448 | 510 | 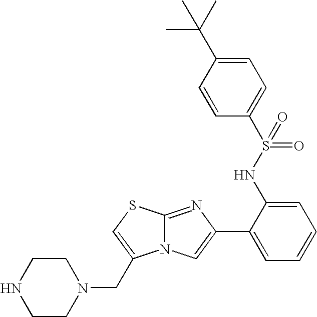 | | NA | |
| 449 | 376 | 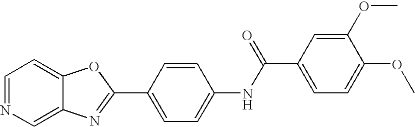 | | NA | |
| 450 | 392 | 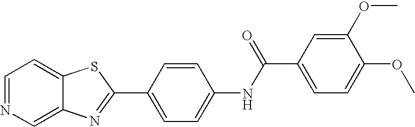 | | NA | |
| 451 | 376 | 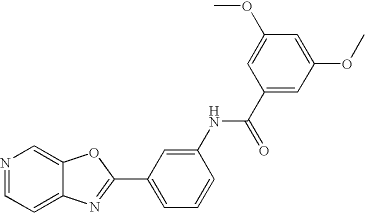 | | A | B |
| 452 | 406 | 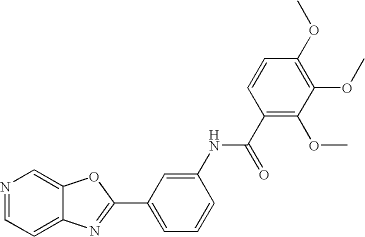 | | A | B |
| 453 | 359 | 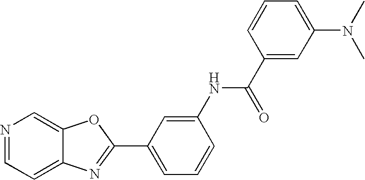 | | A | B |
| 454 | 376 | 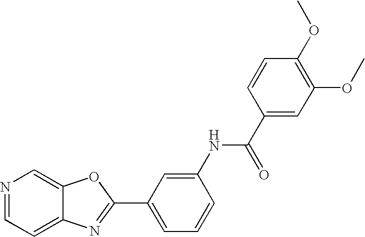 | | A | B |
| 455 | 376 | 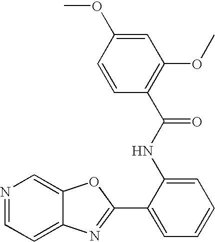 | | A′ | B |
| 456 | 376 | 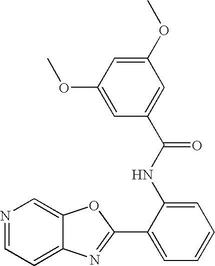 | | A′ | B |
| 457 | 406 | 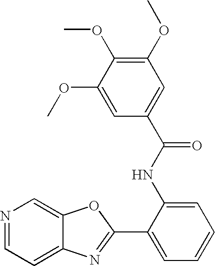 | | A | B |
| 458 | 359 | 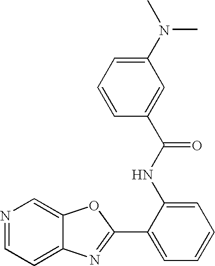 | | A′ | B |
| 459 | 359 | 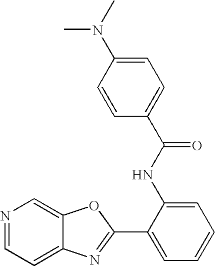 | | A′ | B |
| 460 | 359.4 | 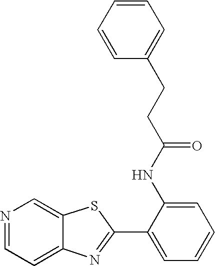 | | A | B |
| 461 | 367.4 | 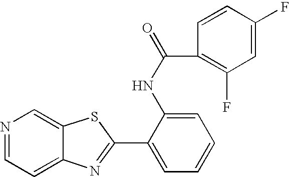 | | NA | |
| 462 | 391.5 | 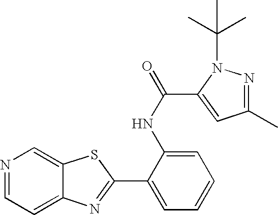 | | A′ | B |
| 463 | 375.4 | 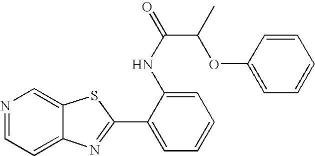 | | A | B |
| 465 | 395.9 | 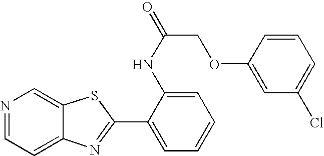 | | NA | |
| 466 | 445.5 | 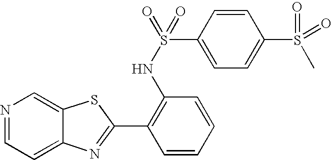 | | NA | |
| 467 | 427.2 | 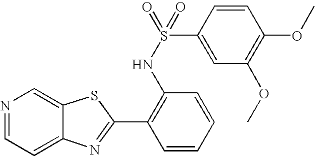 | | NA | |
| 468 | 345.0 | 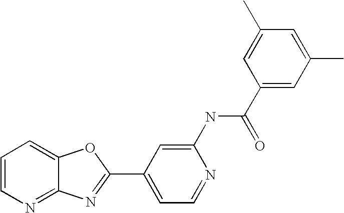 | | A | B |
| 469 | 435.4 | 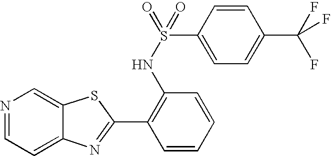 | | NA | |
| 470 | 365.0 | 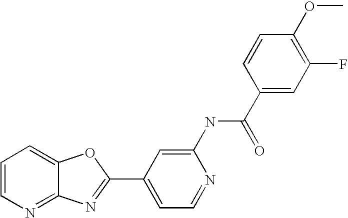 | | NA | |
| 471 | 488.9 | 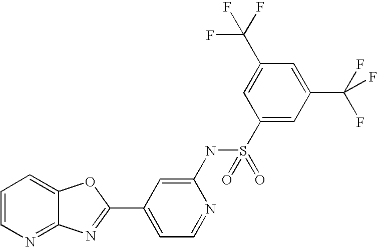 | | NA | |
| 472 | 437.5 | 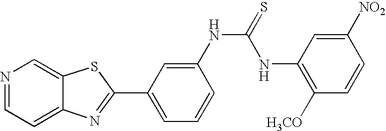 | | NA | |
| 473 | 420.5 | 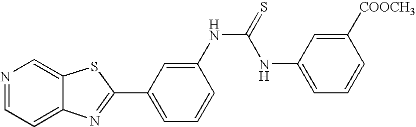 | | A′ | B |
| 474 | | 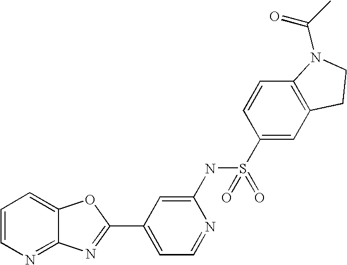 | | NA | |
| 475 | 408.6 | 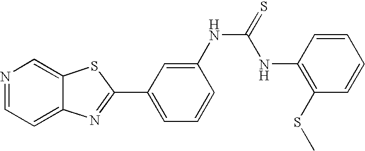 | | NA | |
| 476 | 410.9 | 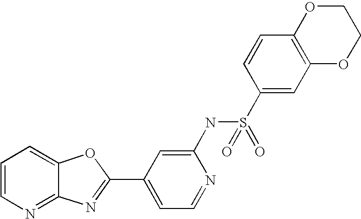 | | NA | |
| 477 | 435.9 | 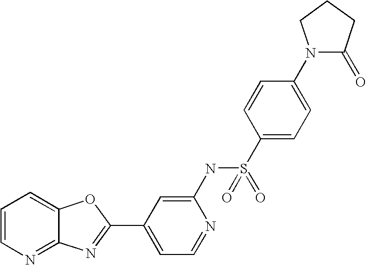 | | NA | |
| 478 | 404.8 | 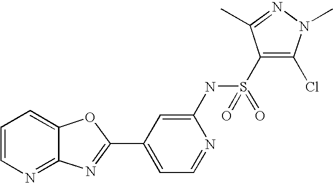 | | NA | |
| 479 | 420.9 | 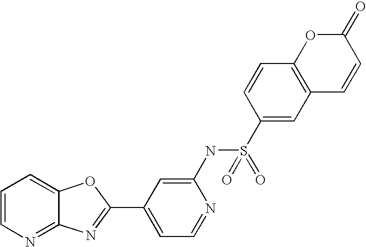 | | NA | |
| 481 | 428.5 | 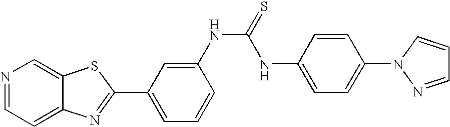 | | A | B |
| 482 | 344.1 | 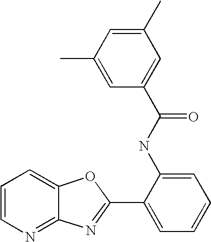 | | A′ | B |
| 483 | 364.1 | 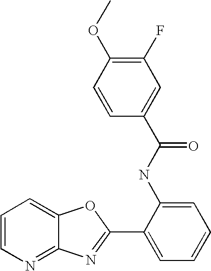 | | NA | |
| 484 | 448.6 | 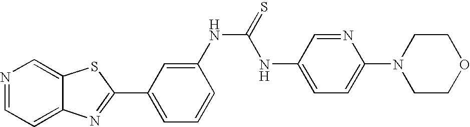 | | A | B |
| 485 | 363.5 | 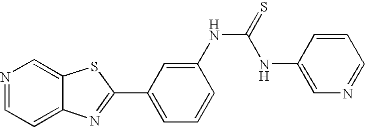 | | A | A |
| 486 | 385.9 | 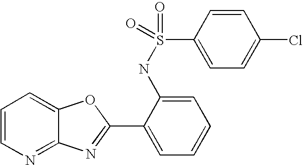 | | NA | |
| 487 | 396.1 | 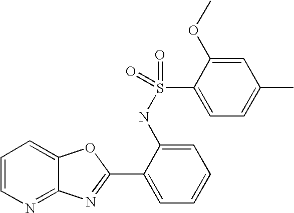 | | NA | |
| 488 | 435.1 | 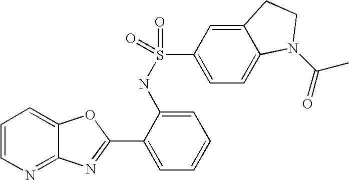 | | NA | |
| 489 | 410.1 | 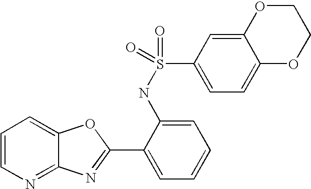 | | NA | |
| 490 | 434.9 | 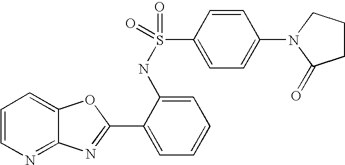 | | NA | |
| 491 | 420.5 | 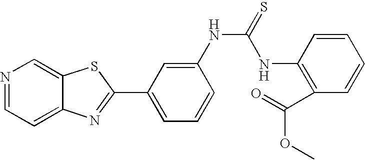 | | NA | |
| 492 | 404.0 | 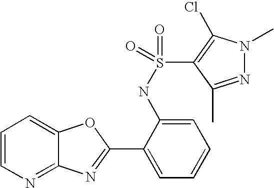 | | NA | |
| 493 | | 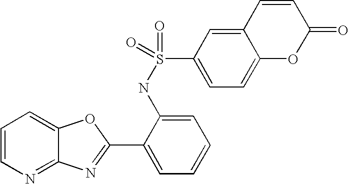 | | NA | |
| 494 | 422.9 | 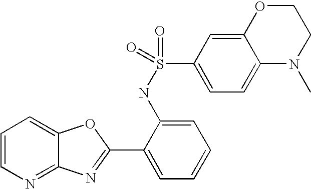 | | NA | |
| 495 | 366.0 | 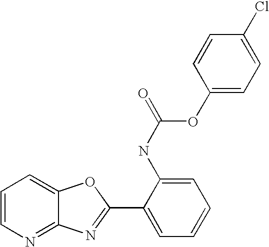 | | NA | |
| 496 | 349.9 | 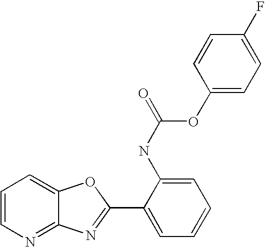 | | NA | |
| 497 | 346.0 | 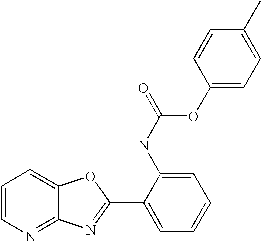 | | NA | |
| 498 | 406.5 | 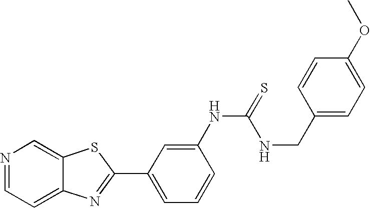 | | A | B |
| 499 | 362.1 | 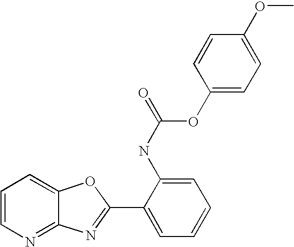 | | NA | |
| 500 | | 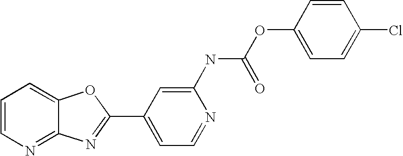 | | NA | |
| 501 | 347.1 | 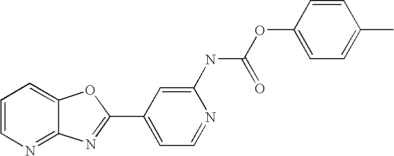 | | NA | |
| 502 | 363.1 | 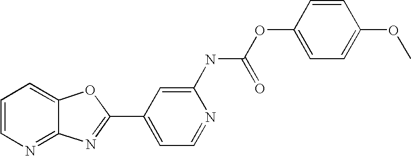 | | NA | |
| 503 | 443.1 | 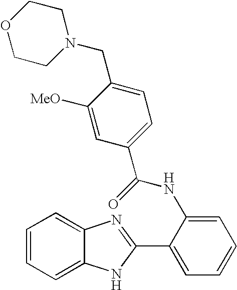 | | A′ | A |
| 504 | 358 | 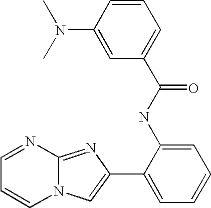 | | A | B |
| 505 | 359 | 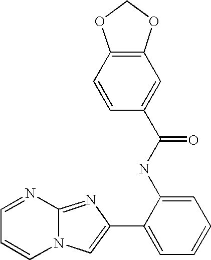 | | NA | |
| 506 | 429.1 | 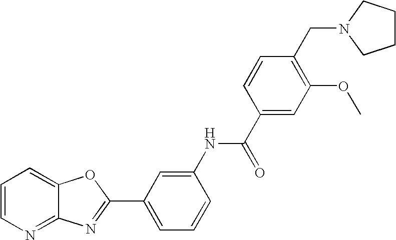 | | A | A |
| 507 | 388.1 | 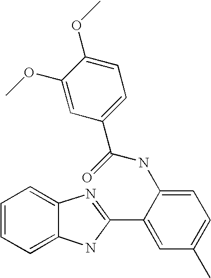 | | A′ | A |
| 508 | 366.1 | 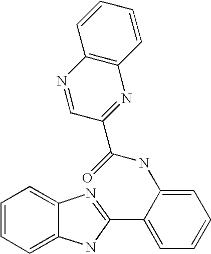 | | A′ | A |
| 510 | 457 | 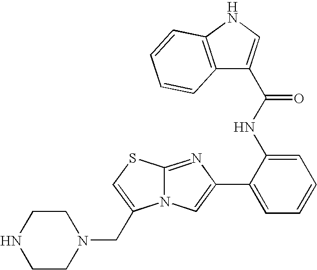 | | A′ | A |
| 511 | 460 | 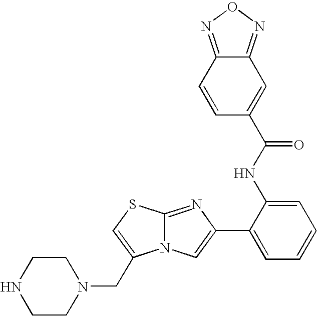 | | NA | |
| 512 | 484 | 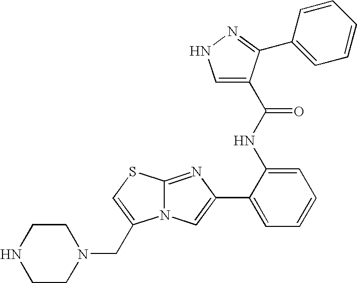 | | A′ | A |
| 513 | 470 | 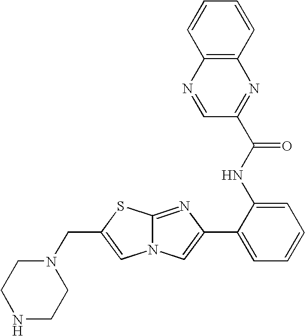 | | A′ | A |
| 514 | 466 | 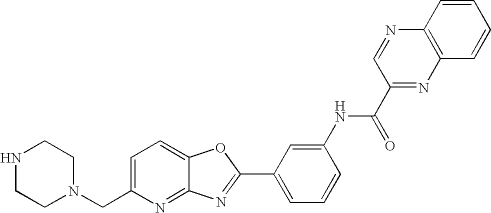 | | A | B |
| 515 | 398 | 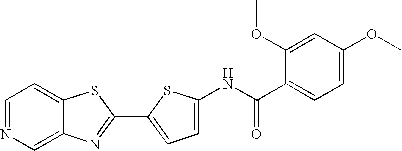 | | A′ | B |
| 516 | 398 | 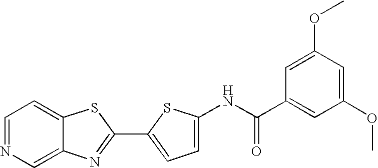 | | NA | |
| 517 | 428 | 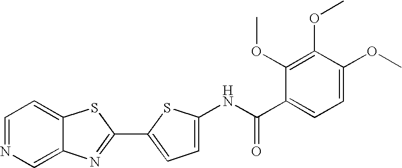 | | NA | |
| 518 | 381 | 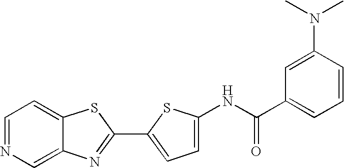 | | A′ | B |
| 519 | 381 | 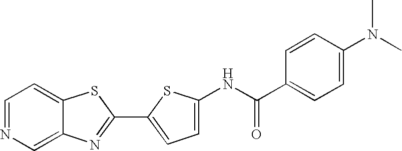 | | A′ | B |
| 520 | 428 | 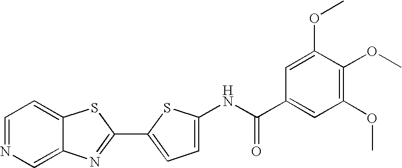 | | A′ | B |
| 521 | 375.0 | 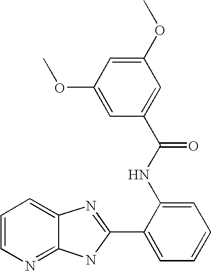 | | A′ | B |
| 522 | 405.0 | 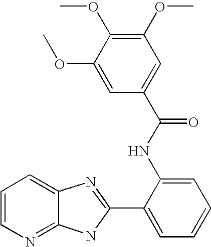 | | A | A |
| 523 | 359.0 | 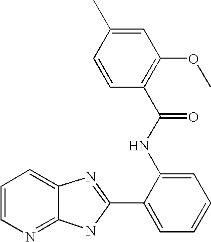 | | A | B |
| 524 | 358.0 | 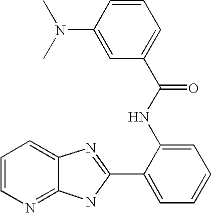 | | A′ | B |
| 525 | 375.0 | 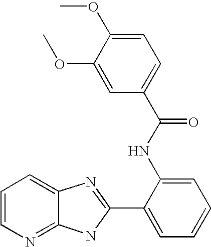 | | A′ | B |
| 526 | 400.0 | 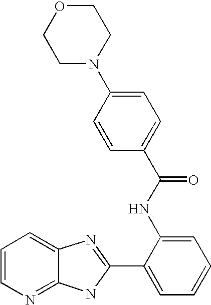 | | A′ | A |
| 527 | 398.0 | 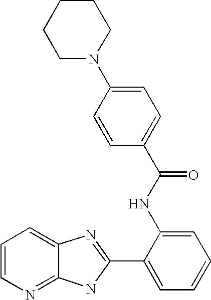 | | A′ | B |
| 528 | 412.9 | 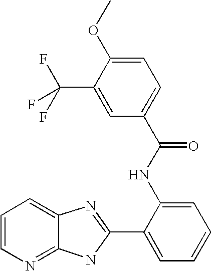 | | A′ | B |
| 529 | 399.1 | 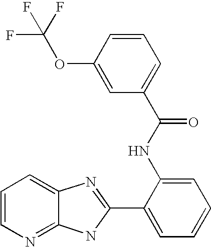 | | A′ | B |
| 530 | 359.0 | 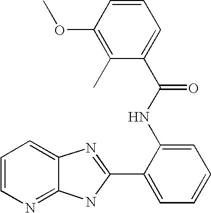 | | A′ | B |
| 531 | 345.0 | 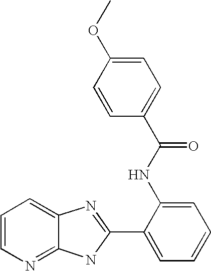 | | A′ | B |
| 532 | 345.0 | 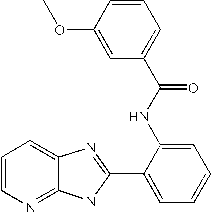 | | A′ | B |
| 533 | 315.0 | 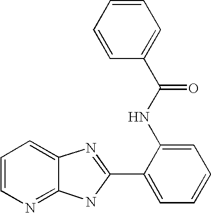 | | A′ | B |
| 534 | 358.0 | 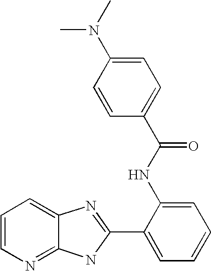 | | A′ | B |
| 535 | 428.1 | 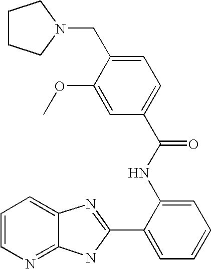 | | A | A |
| 536 | 442.1 | 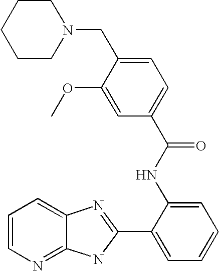 | | A | A |
| 537 | 444.0 | 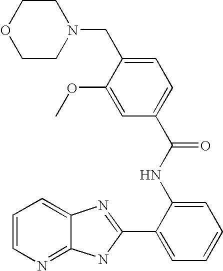 | | A′ | A |
| 538 | 443.1 | 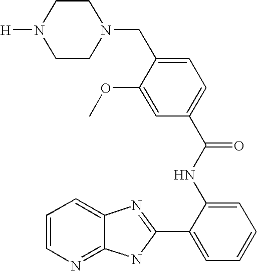 | | A | A |
| 539 | 457.1 | 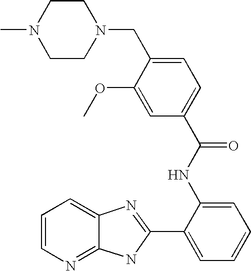 | | A′ | A |
| 540 | 402.1 | 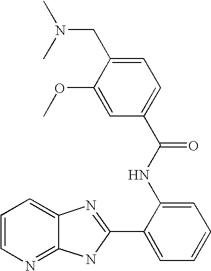 | | A | A |
| 541 | 443.1 | 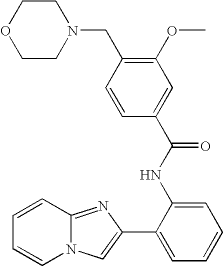 | | A | B |
| 542 | 444 | 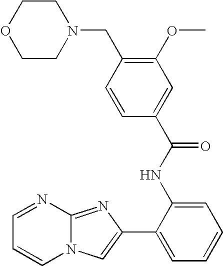 | | A | A |
| 543 | 420 | 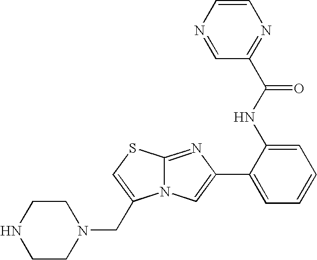 | | A′ | A |
| 544 | 474 | 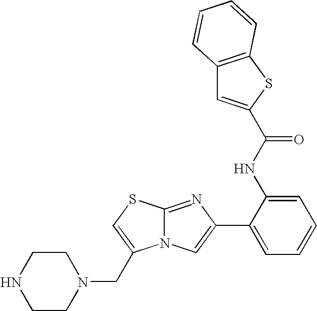 | | A | A |
| 545 | 378.1 | 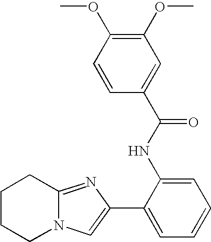 | | A | B |
| 546 | 408 | 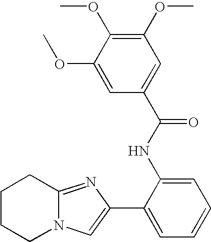 | | A | B |
| 547 | 369 | 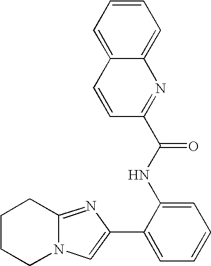 | | A′ | B |
| 548 | 370 | 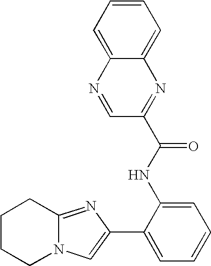 | | A′ | B |
| 556 | 365.1 | 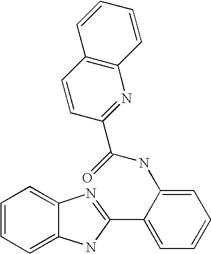 | | A′ | A |
| 557 | 542.1 | 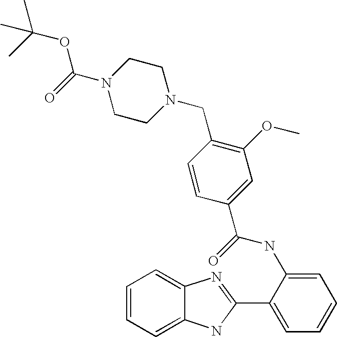 | | NA | |
| 558 | 442.1 | 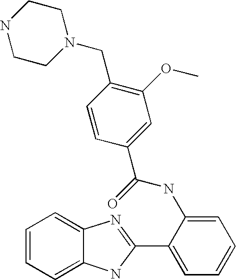 | | A′ | A |
| 559 | 508 | 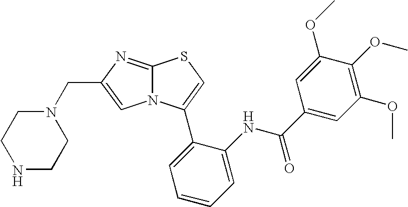 | | NA | |
| 560 | 470 | 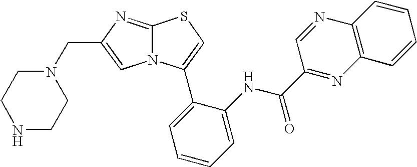 | | NA | |
| 561 | 382 | 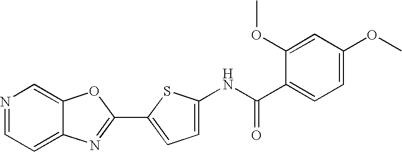 | | A′ | B |
| 562 | 382 | 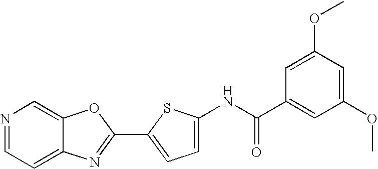 | | A′ | B |
| 563 | 412 | 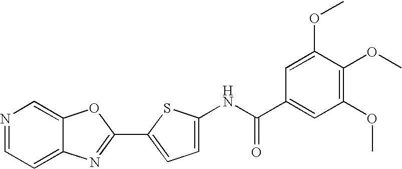 | | A | B |
| 565 | 400.8 | 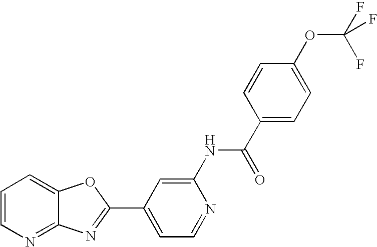 | | NA | |
| 566 | 409.9 | 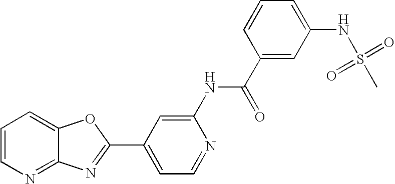 | | A | B |
| 567 | 374.9 | 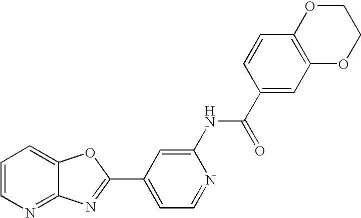 | | NA | |
| 568 | 367.0 | 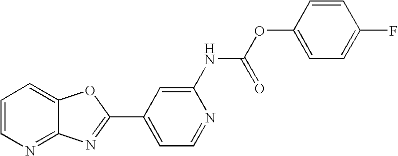 | | NA | |
| 569 | 374.9 | 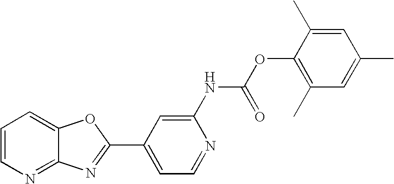 | | NA | |
| 570 | 361.0 | 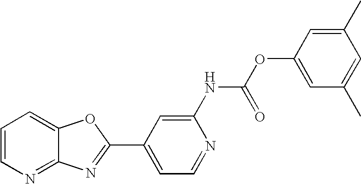 | | NA | |
| 571 | 444.0 | 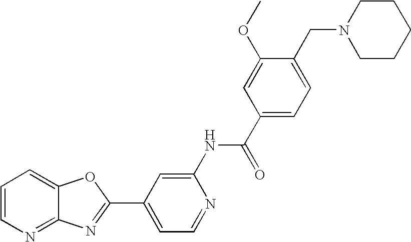 | | A | B |
| 572 | 429.9 | 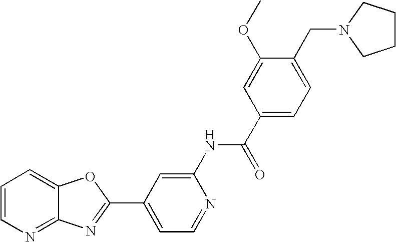 | | B | B |
| 573 | 431.8 | 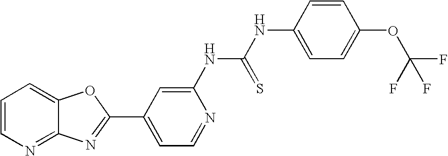 | | NA | |
| 574 | 376.2 | 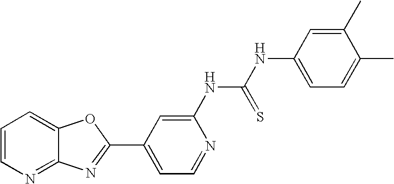 | | NA | |
| 575 | 439.9 | 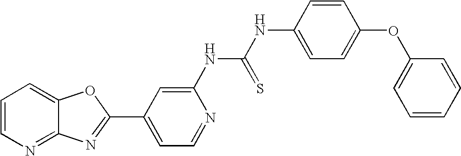 | | NA | |
| 576 | 376.1 | 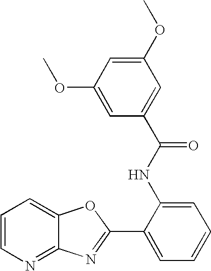 | | NA | |
| 577 | 400.1 | 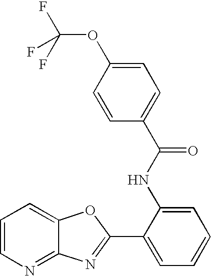 | | NA | |
| 578 | 368.1 | 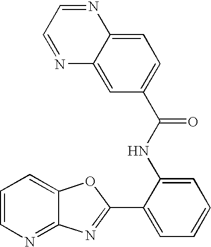 | | NA | |
| 579 | 401.1 | 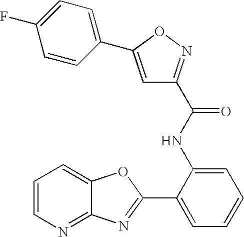 | | NA | |
| 580 | 374.0 | 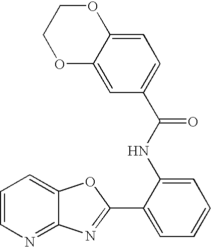 | | NA | |
| 581 | 334.0 | 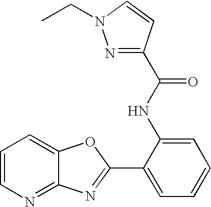 | | A | B |
| 582 | 400.0 | 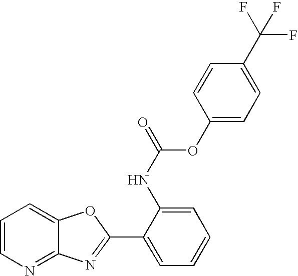 | | NA | |
| 583 | 374.1 | 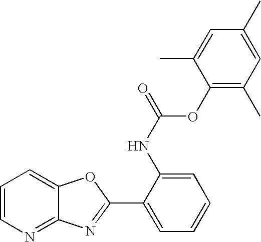 | | NA | |
| 584 | 360.0 | 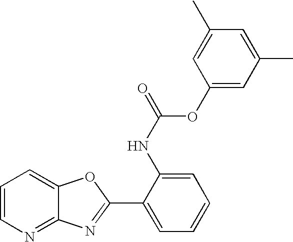 | | NA | |
| 585 | 443.1 | 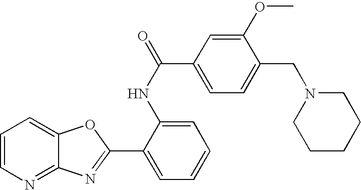 | | A | B |
| 587 | 427.1 | 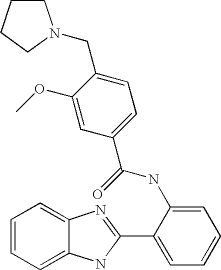 | | A′ | B |
| 588 | 520 | 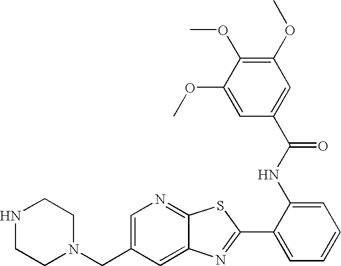 | | A | A |
| 589 | 490 | 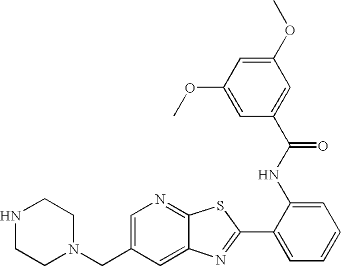 | | A′ | A |
| 590 | 474 | 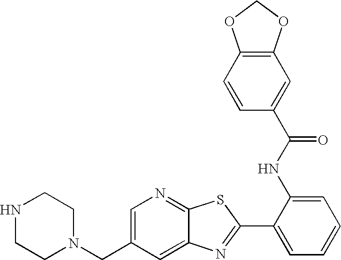 | | A | B |
| 591 | 458 | 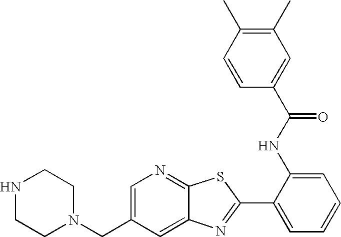 | | A′ | A |
| 592 | 455 | 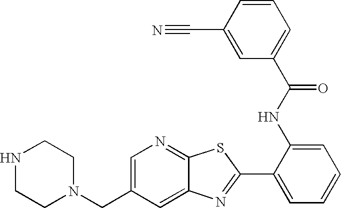 | | A′ | B |
| 593 | 473 | 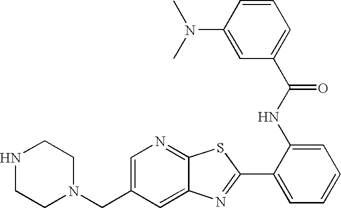 | | A′ | A |
| 594 | 466 | 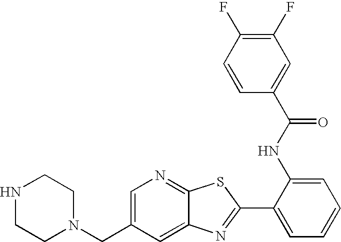 | | A | B |
| 596 | 467.4 | 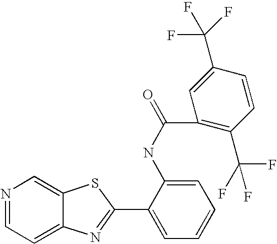 | | A′ | B |
| 597 | 377.2 | 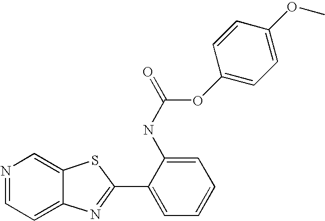 | | A′ | B |
| 599 | 422.3 | 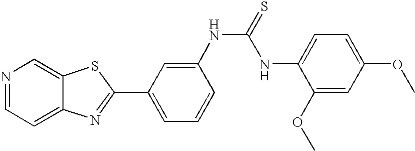 | | A′ | B |
| 600 | 422.5 | 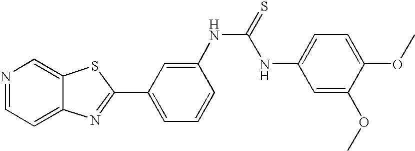 | | D | B |
| 601 | 405.5 | 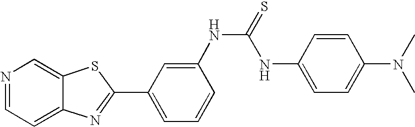 | | NA | |
| 604 | 360.4 | 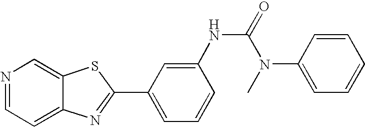 | | NA | |
| 605 | 377.4 | 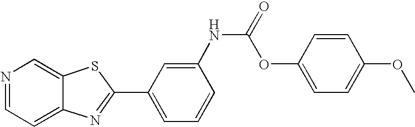 | | NA | |
| 607 | 415.4 | 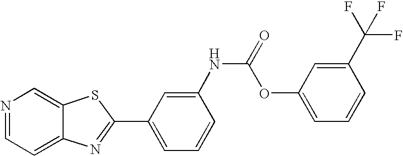 | | NA | |
| 608 | 332.4 | 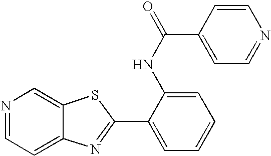 | | NA | |
| 609 | 439.3 | 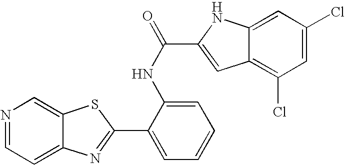 | | NA | |
| 610 | 418.6 | 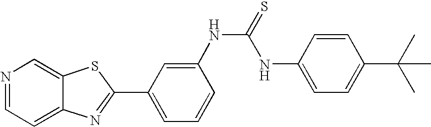 | | NA | |
| 611 | 465.6 | 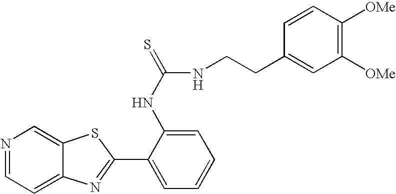 | | A′ | B |
| 612 | 402.5 | 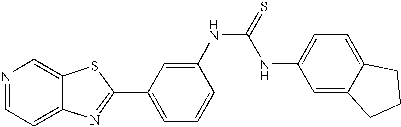 | | NA | |
| 614 | 428.5 | 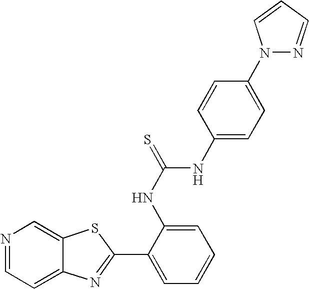 | | NA | |
| 615 | 408.6 | 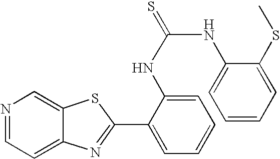 | | NA | |
| 616 | 437.5 | 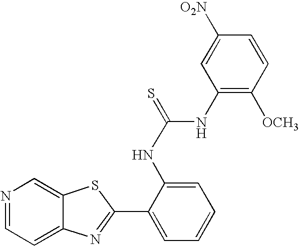 | | | |
| 617 | 471.1 | 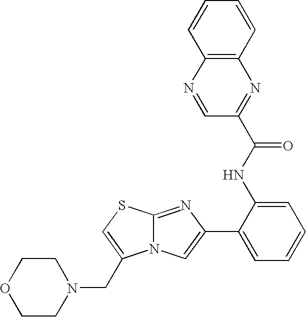 | | A′ | A |
| 618 | 469.1 | 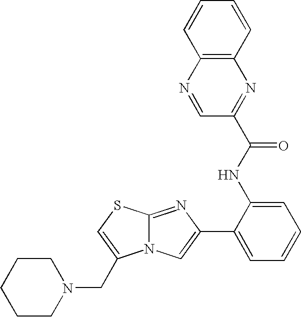 | | A′ | B |
| 619 | 455 | 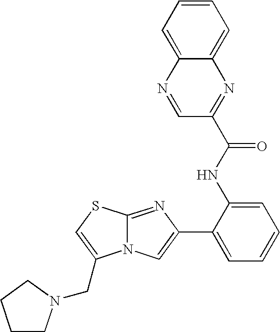 | | A′ | B |
| 620 | 493.1 | 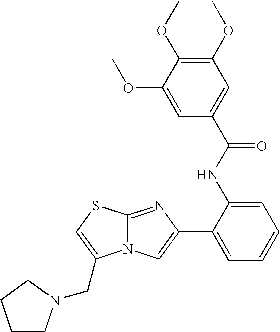 | | A′ | B |
| 621 | 472 | 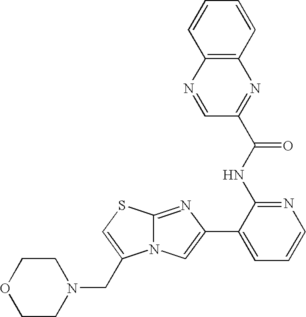 | | A′ | A |
| 622 | 482 | 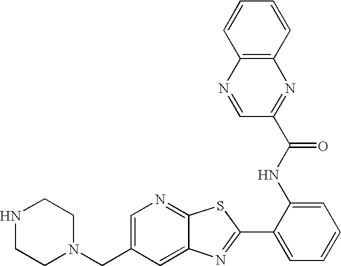 | | A′ | A |
| 623 | 437 | 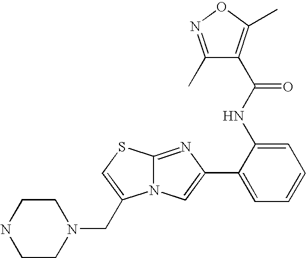 | | NA | C |
| 624 | 458 | 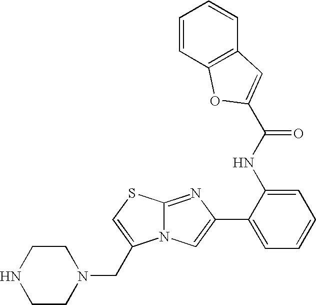 | | A′ | A |
| 625 | 496 | 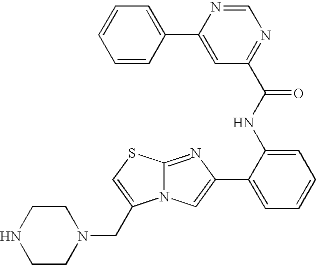 | | A′ | A |
| 628 | 574.1 | 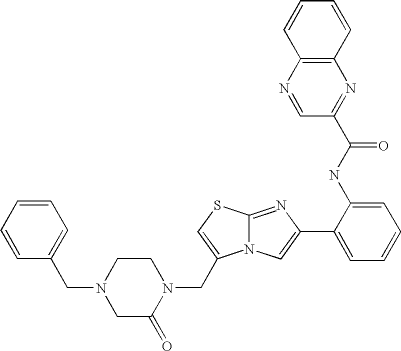 | | A′ | B |
| 629 | 429.0 | 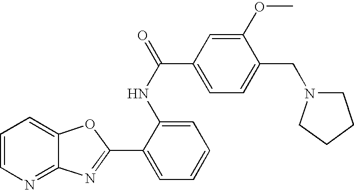 | | A | A |
| 630 | 403.1 | 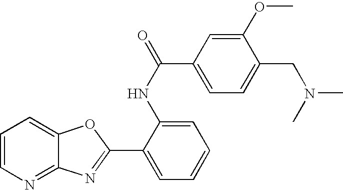 | | A | A |
| 631 | 445.0 | 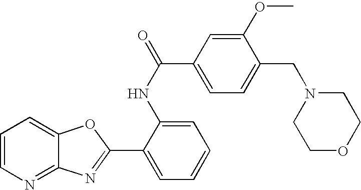 | | A′ | B |
| 632 | 458.1 | 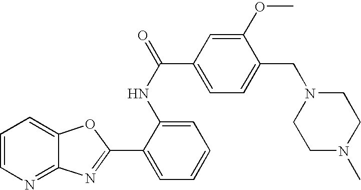 | | A | A |
| 633 | 423.3 | 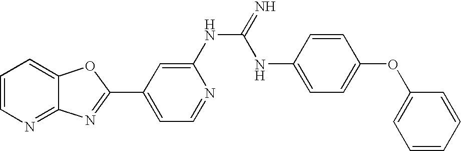 | | NA | C |
| 634 | 364.9 | 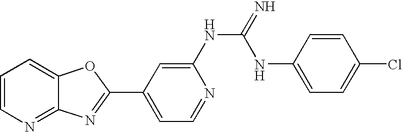 | | D | B |
| 635 | 421.1 | 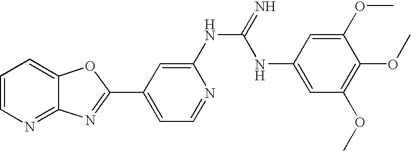 | | D | A |
| 636 | 359.9 | 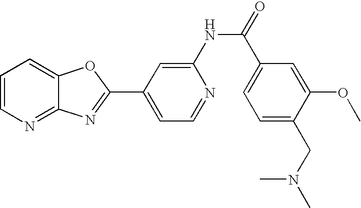 | | B | B |
| 637 | 459.3 | 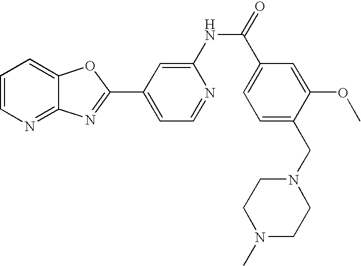 | | D | B |
| 638 | 445.2 | 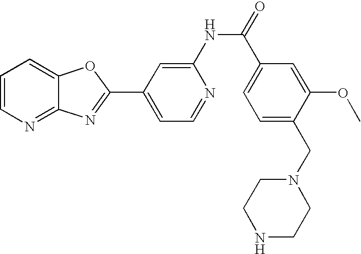 | | D | B |
| 639 | 378.9 | 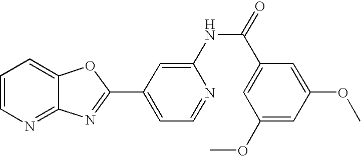 | | A | B |
| 640 | 368.9 | 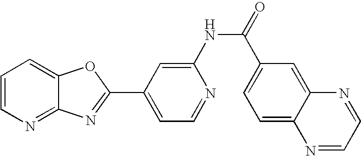 | | NA | C |
| 641 | 334.9 | 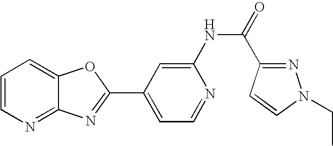 | | D | B |
| 642 | 446.2 | 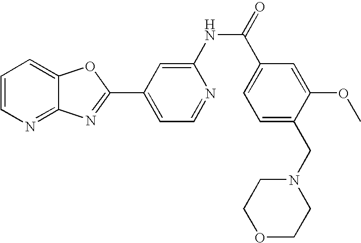 | | D | B |
| 643 | 535.1 | 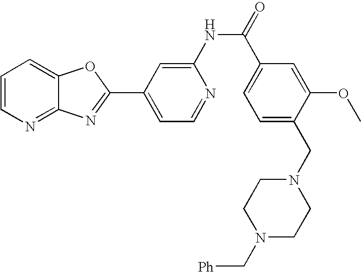 | | A | B |
| 644 | 434 | 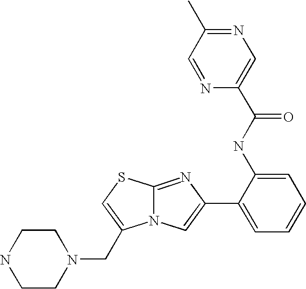 | | A | A |
| 645 | 469 | 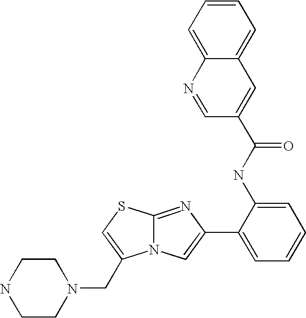 | | A′ | A |
| 646 | 481 | 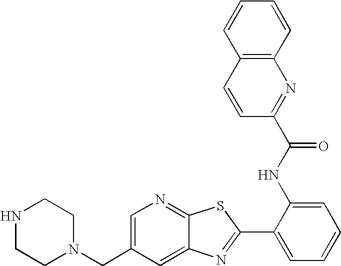 | | A | A |
| 647 | 473.1 | 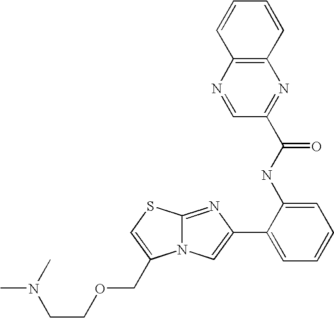 | | A′ | A |
| 648 | 402.1 | 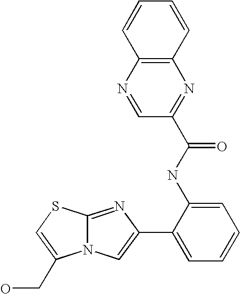 | | A′ | B |
| 649 | 512.2 | 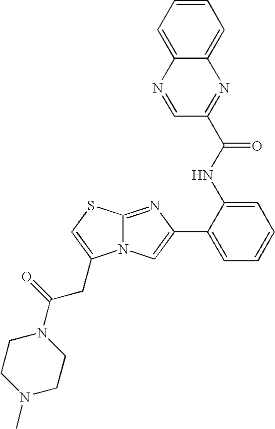 | | A′ | A |
| 650 | 570.2 | 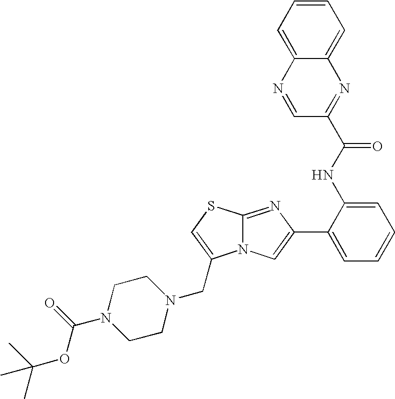 | | NA | |
| 651 | 512.2 | 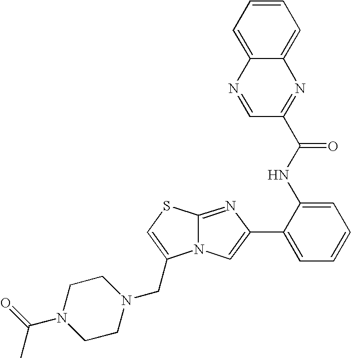 | | A′ | A |
| 655 | 393.0 | 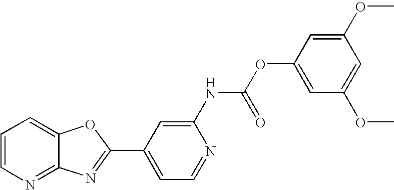 | | NA | C |
| 656 | 393.2 | 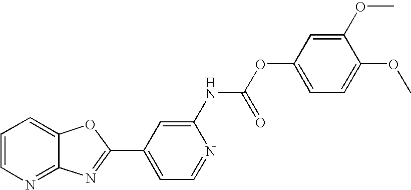 | | NA | C |
| 657 | 423.1 | 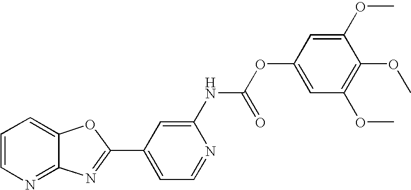 | | NA | C |
| 658 | 382.1 | 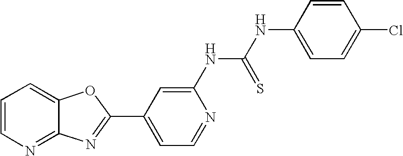 | | NA | C |
| 659 | 509.2 | 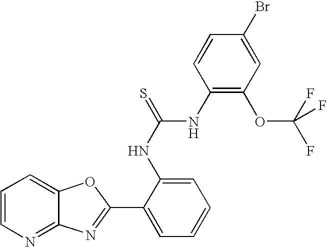 | | NA | C |
| 660 | 408.1 | 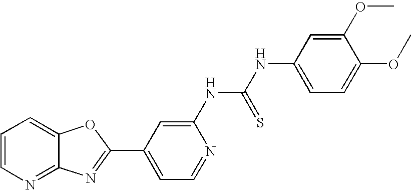 | | A | B |
| 661 | 408.2 | 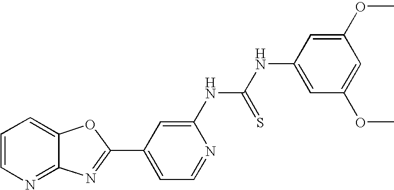 | | A′ | B |
| 662 | 378.2 | 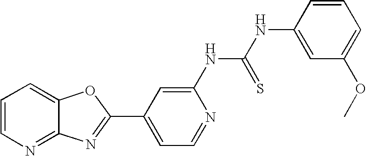 | | NA | C |
| 663 | 438.0 | 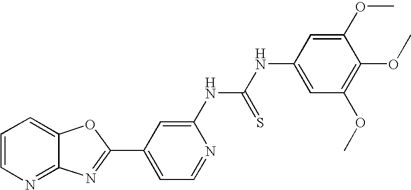 | | A | B |
| 664 | 477.8 | 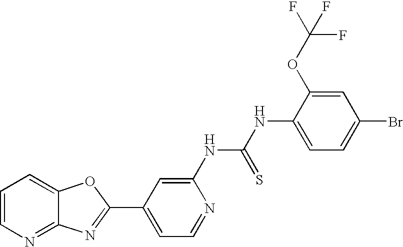 | | NA | C |
| 665 | 406.1 | 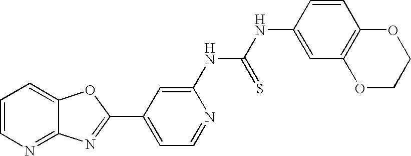 | | NA | C |
| 666 | 391.0 | 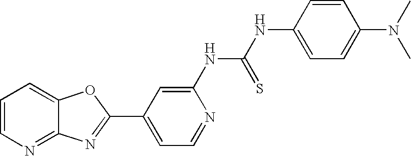 | | NA | C |
| 667 | 448.0 | 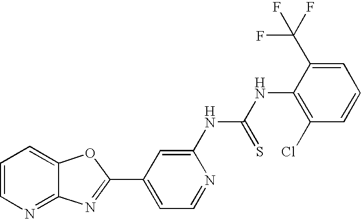 | | A | A |
| 668 | 410.0 | 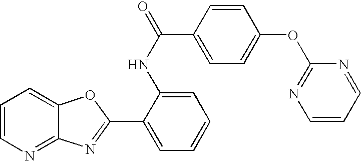 | | A | B |
| 669 | 392.0 | 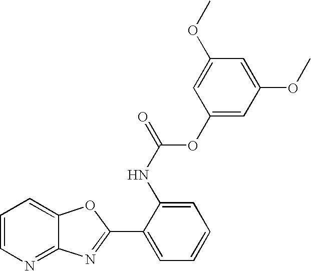 | | NA | C |
| 670 | 392.0 | 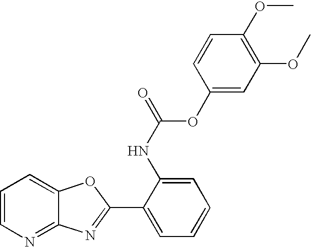 | | A | B |
| 671 | 422.0 | 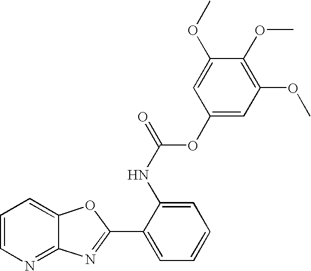 | | NA | C |
| 672 | 415.1 | 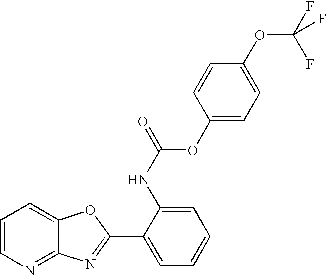 | | NA | C |
| 673 | | 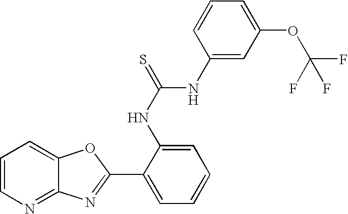 | | NA | C |
| 674 | | 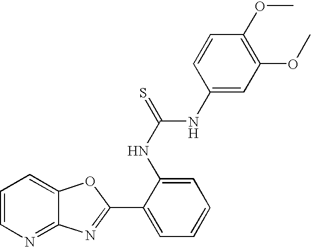 | | NA | C |
| 675 | 407.1 | 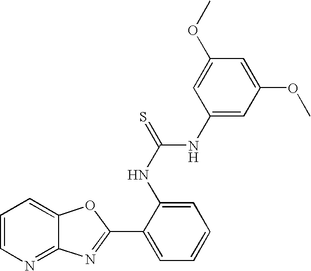 | | NA | C |
| 676 | | 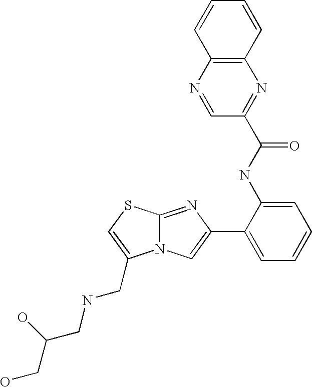 | | A′ | A |
| 677 | | 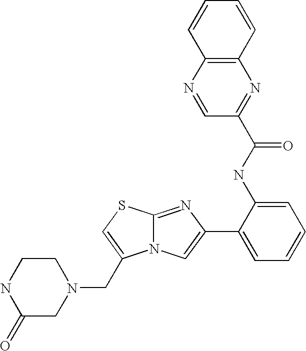 | | A′ | A |
| 678 | | 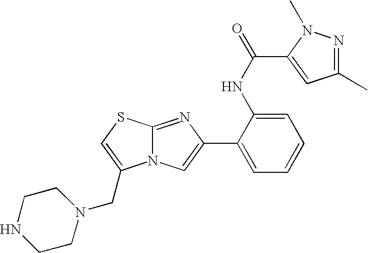 | | A | B |
| 679 | | 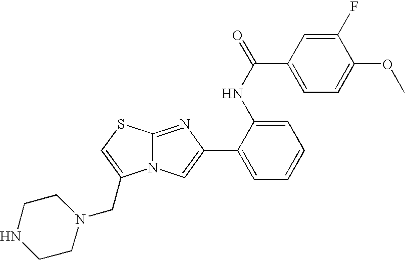 | | A | B |
| 680 | 484.2 | 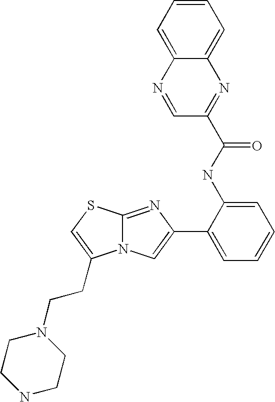 | | A′ | A |
| 681 | 522 | 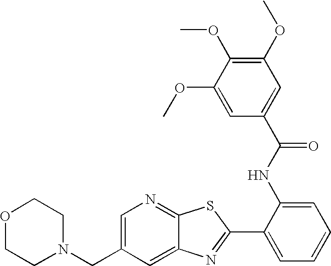 | | A | A |
| 682 | 492 | 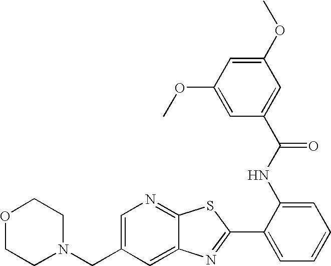 | | A′ | A |
| 683 | 475 | 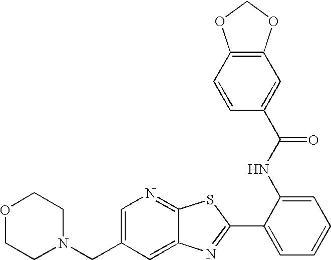 | | A | B |
| 684 | 460 | 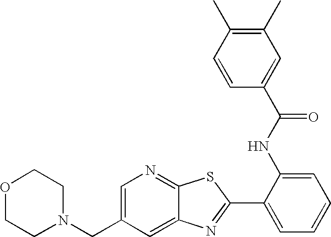 | | A | B |
| 685 | 456 | 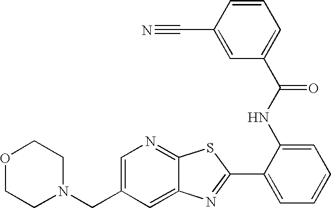 | | A′ | B |
| 686 | 475 | 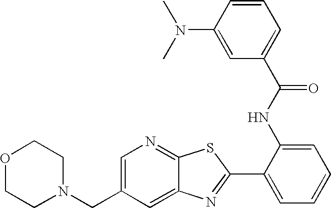 | | A′ | A |
| 687 | 467 | 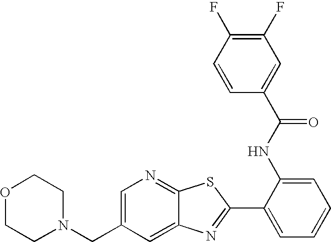 | | NA | C |
| 688 | 483 | 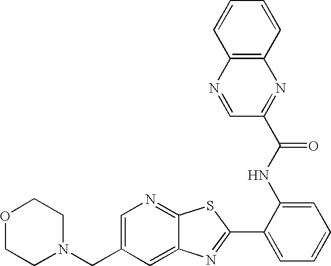 | | NA | C |
| 689 | 479 | 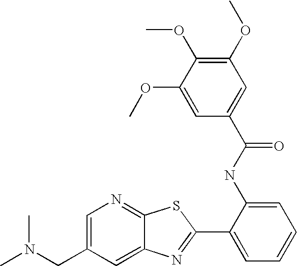 | | A | A |
| 690 | 483 | 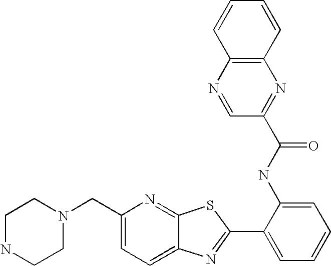 | | A′ | A |
| 692 | 469 | 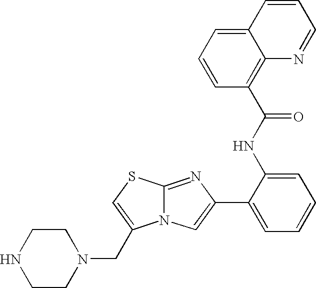 | | C | B |
| 695 | 454 | 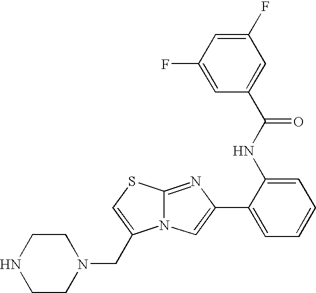 | | A′ | A |
| 697 | 596 | 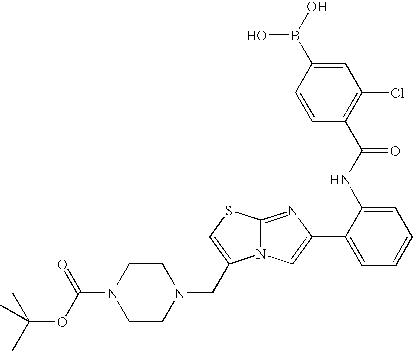 | | NA | C |
| 698 | 502 | 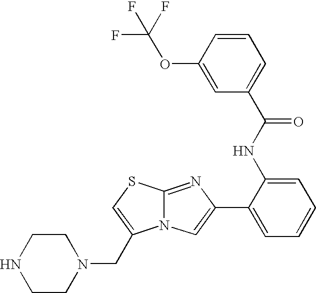 | | A′ | A |
| 699 | | 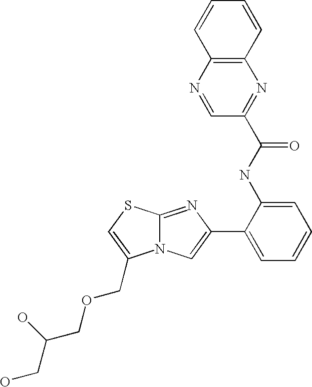 | | A | A |
| 700 | 512.2 | 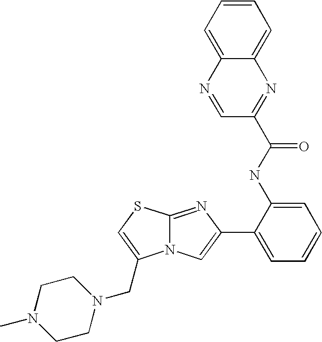 | | A′ | A |
| 701 | 477 | 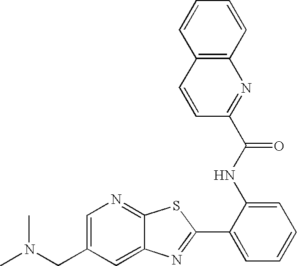 | | A′ | A |
| 702 | 534 | 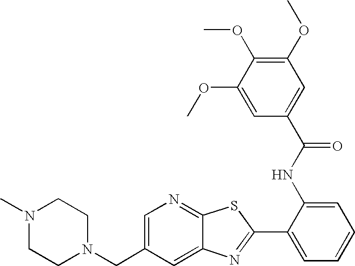 | | A | A |
| 703 | 468 | 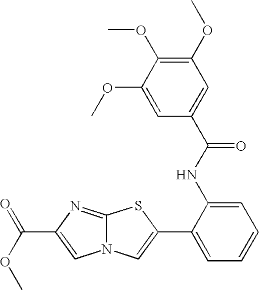 | | NA | C |
| 704 | 454 | 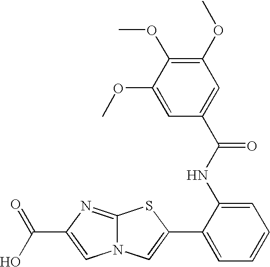 | | NA | C |
| 705 | 387 | 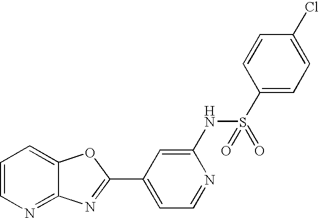 | | | |
| 706 | 445.1 | 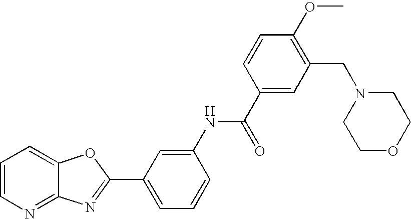 | | | |
| 707 | 386.1 | 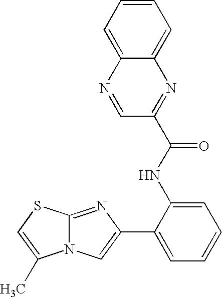 | | A′ | A |
| 708 | 494.2 | 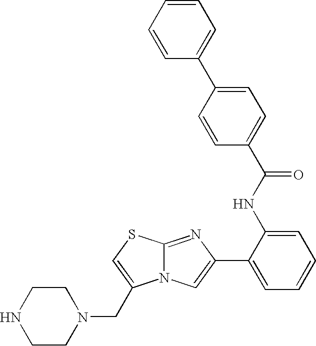 | | A′ | A |
| 709 | 494.1 | 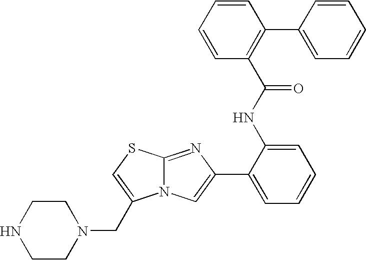 | | NA | C |
| 710 | 494.1 | 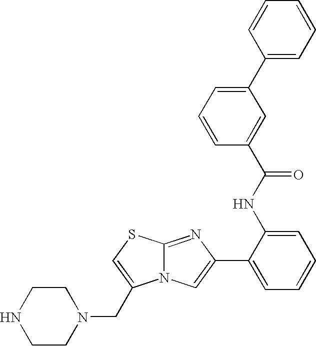 | | A′ | A |
| 711 | | 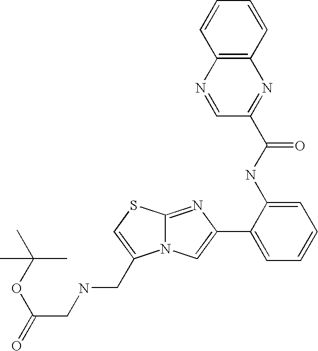 | | A | B |
| 714 | | 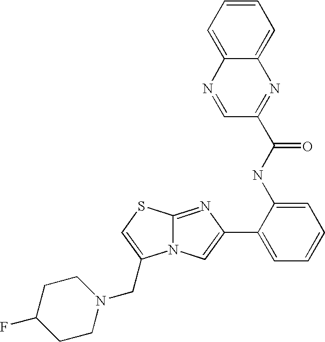 | | A | B |
| 715 | | 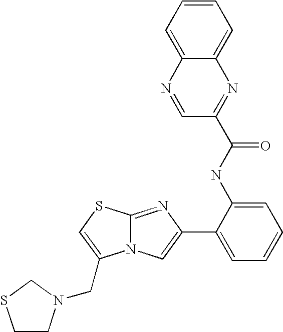 | | A′ | B |
| 716 | | 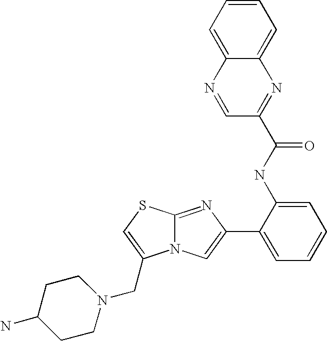 | | A′ | A |
| 717 | | 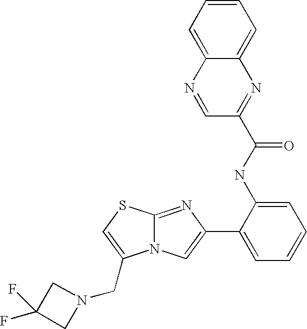 | | A | B |
| 718 | 444.1 | 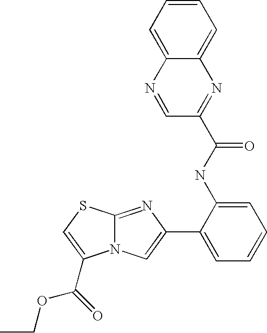 | | NA | C |
| 719 | 416 | 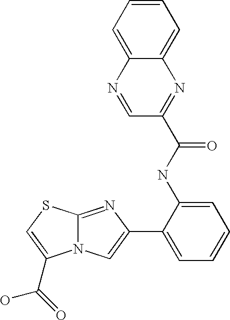 | | A | A |
| 720 | | 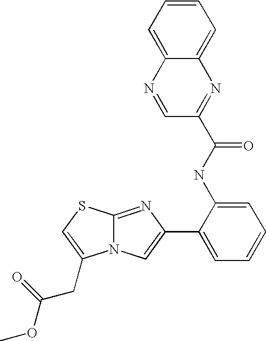 | | NA | C |
| 721 | | 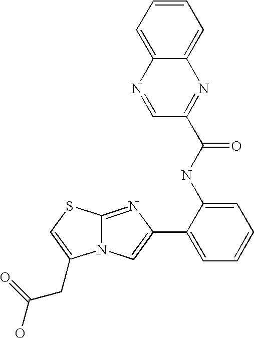 | | A | A |
| 722 | 449 | 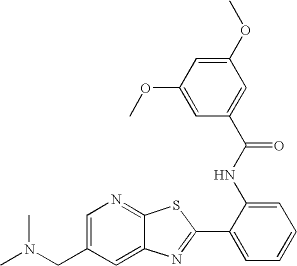 | | A′ | A |
| 723 | 490 | 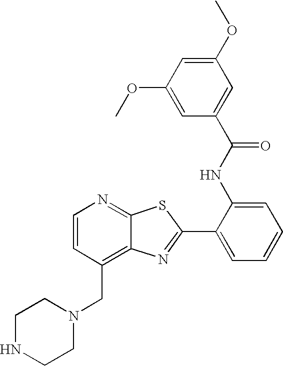 | | A | A |
| 724 | 482 | 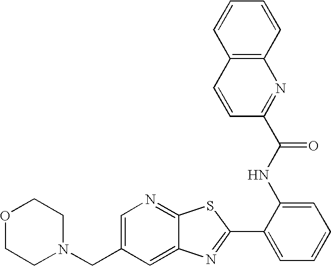 | | A′ | A |
| 725 | 505 | 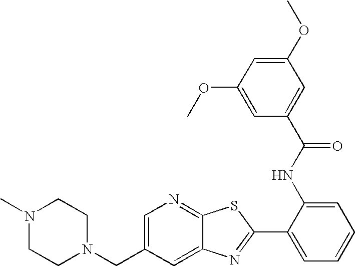 | | A′ | A |
| 726 | 491 | 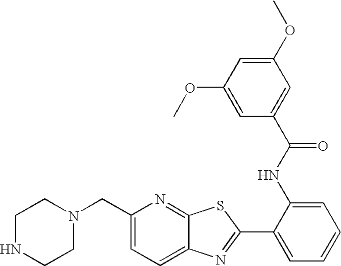 | | A′ | A |
| 727 | 458 | 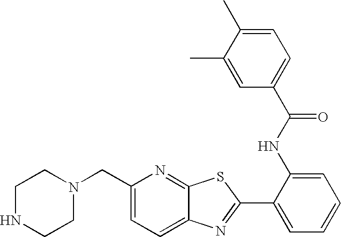 | | A′ | A |
| 728 | 466 | 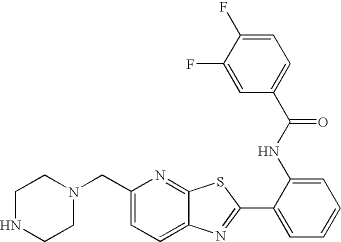 | | A | A |
| 729 | 481 | 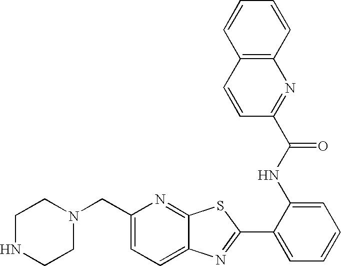 | | A′ | A |
| 730 | 488 | 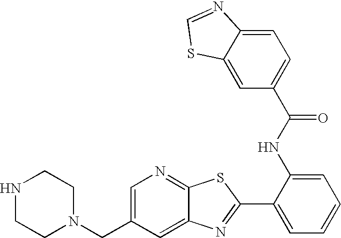 | | A | A |
| 731 | 498 | 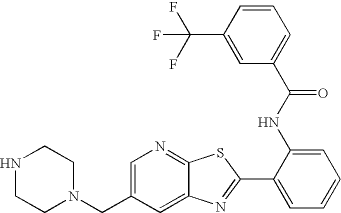 | | A | A |
| 732 | 497 | 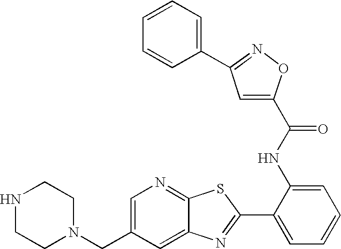 | | A′ | B |
| 733 | 484.2 | 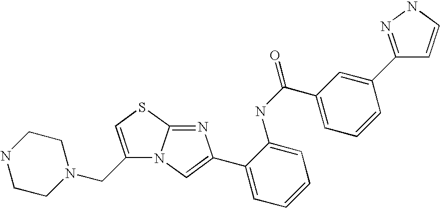 | | A′ | A |
| 735 | 514.2 | 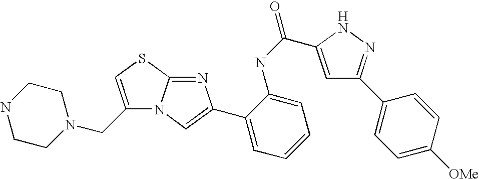 | | A′ | A |
| 736 | 514.2 | 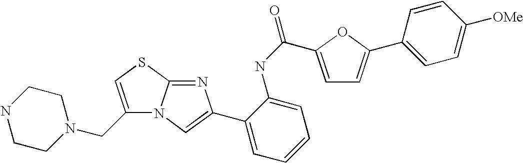 | | A′ | A |
| 737 | 500.1 | 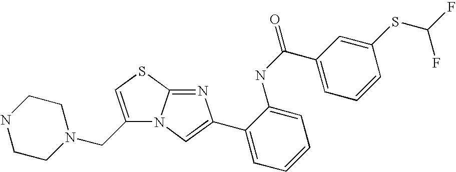 | | A′ | A |
| 738 | 448.1 | 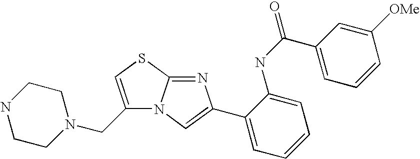 | | A | A |
| 739 | 424.1 | 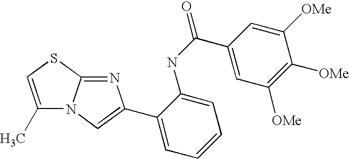 | | A′ | A |
| 740 | 377.1 | 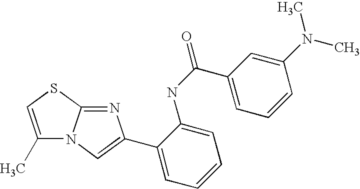 | | A′ | B |
| 741 | 498.1 | 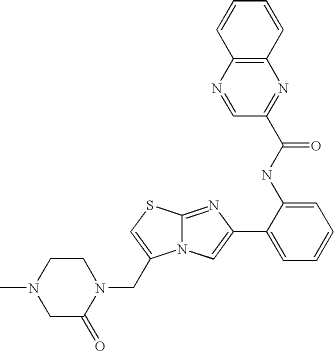 | | A′ | A |
| 742 | 487.1 | 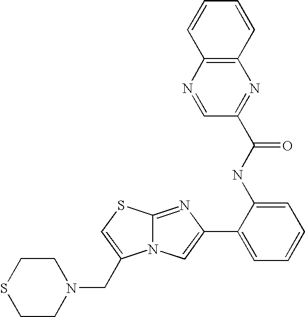 | | A′ | B |
| 743 | 466.1 | 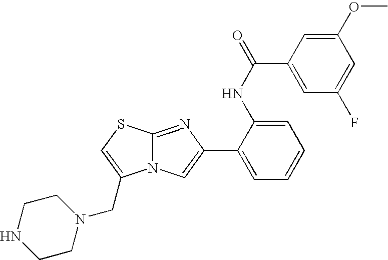 | | A | A |
| 744 | 437.1 | 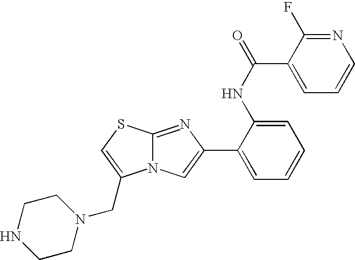 | | B | A |
| 745 | 452.1 | 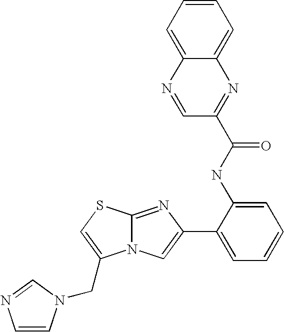 | | A′ | A |

TABLE 5

| COMPOUND | | | IC₅₀ FP | IC₅₀ MS |
|---|---|---|---|---|
| No | [M + H]+ | STRUCTURE | ASSAY | ASSAY |
| 13 | | 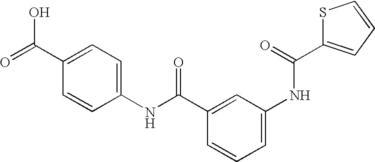 | A | |
| 14 | | 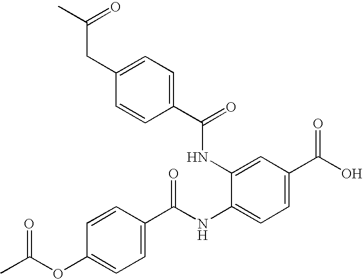 | B | |
| 15 | | 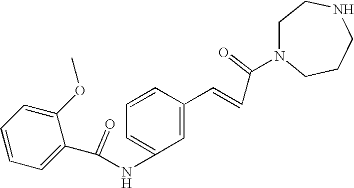 | C | |
| 23 | 359.4 | 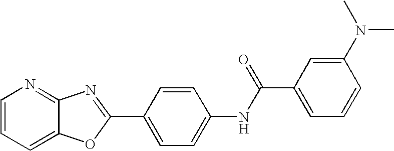 | D | |
| 25 | 341.3 | 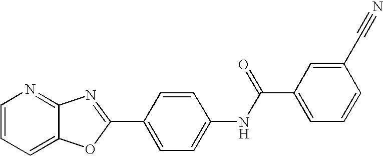 | B | |
| 26 | 341.4 | 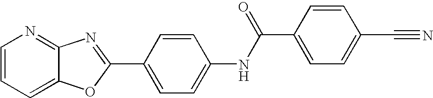 | B | |
| 28 | 401.1 | 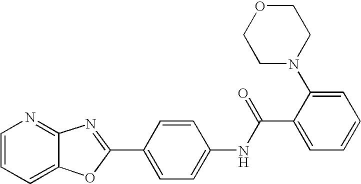 | D | |
| 30 | 380.0 | 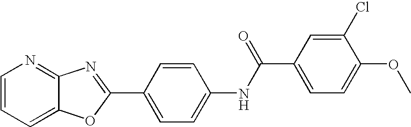 | D | |
| 44 | 341.0 | 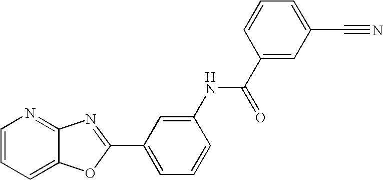 | D | |
| 47 | 385.0 | 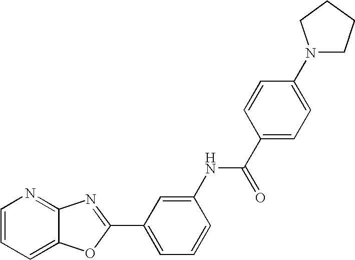 | B | |
| 291 | | 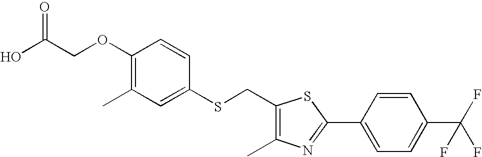 | B | |
| 652 | | 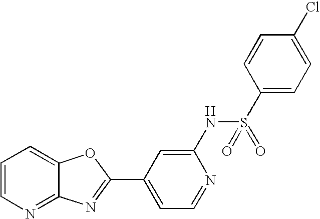 | | B |
| 653 | | 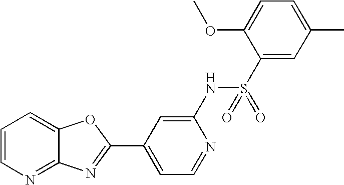 | | C |
| 654 | | 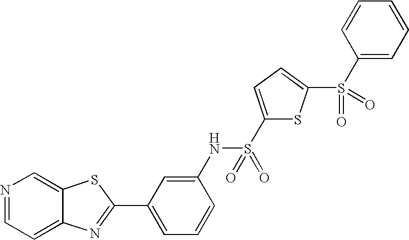 | | D |

TABLE 6

| COMPOUND No | STRUCTURE | ED₅₀ |
|---|---|---|
| 11 | 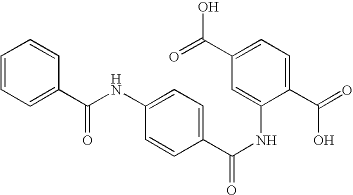 | N/A |
| 12 | 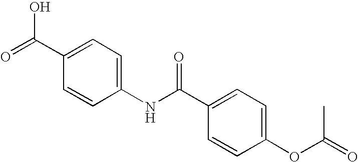 | N/A |

TABLE 7

| COMPOUND No | STRUCTURE | IC₅₀ |
|---|---|---|
| 1 | 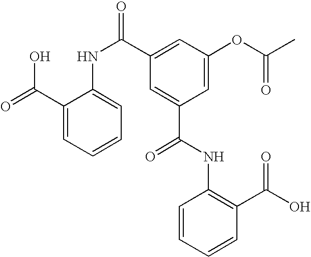 | N/A |
| 2 | 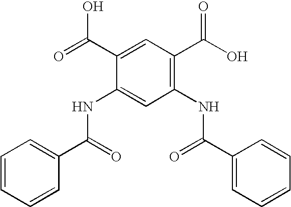 | N/A |
| 15 | 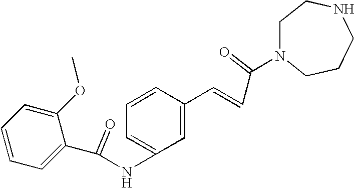 | B |
| 16 | 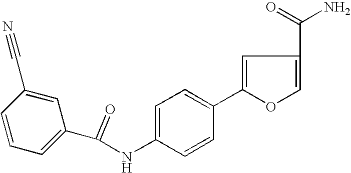 | C |
| 17 | 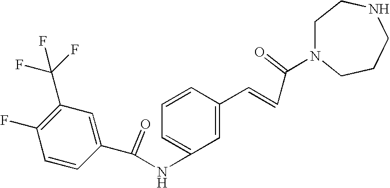 | B |
| 18 | 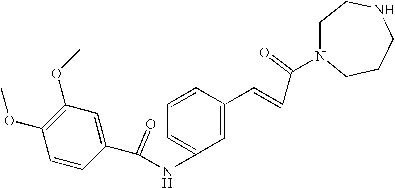 | B |

TABLE 8

| COMPOUND | | ED₅₀ ATP | IC₅₀ ATP |
|---|---|---|---|
| No | STRUCTURE | ASSAY | ASSAY |
| 52 | 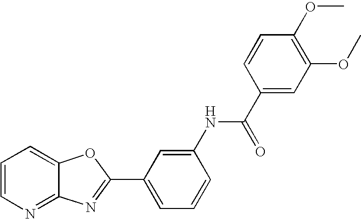 | A | |
| 42 | 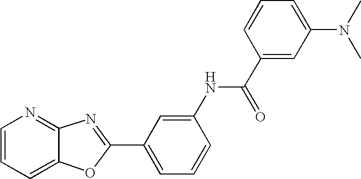 | A | |
| 49 | 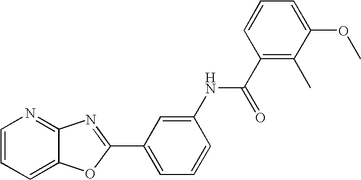 | A | |
| 115 | 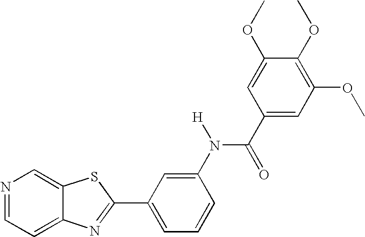 | D | |
| 79 | 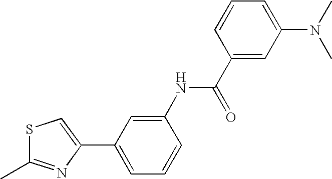 | A | |
| 117 | 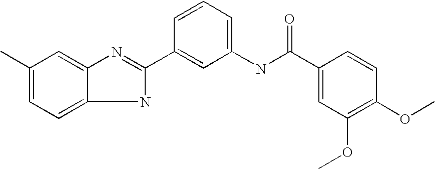 | | B |
| 120 | 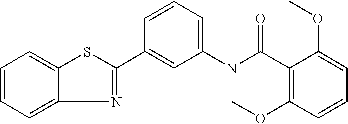 | A | |
| 121 | 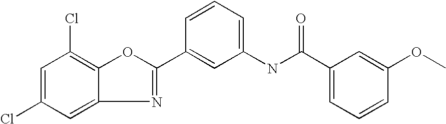 | NA | |
| 123 | 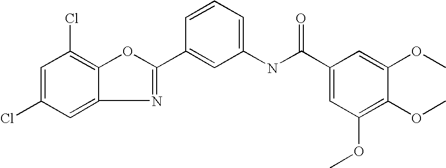 | D | |
| 85 | 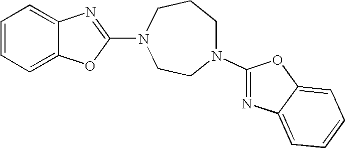 | NA | |
| 86 | 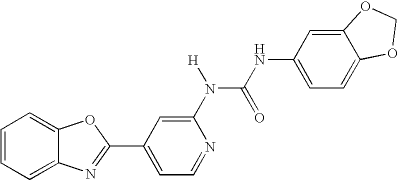 | A | |
| 87 | 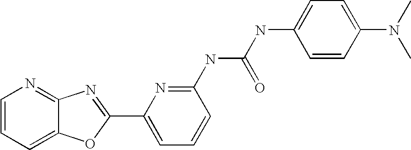 | NA | |
| 88 | 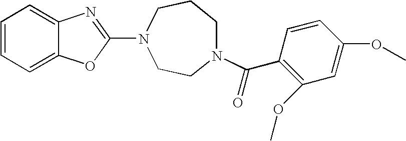 | NA | |
| 89 | 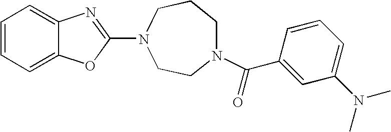 | | A |
| 90 | 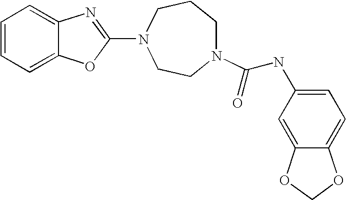 | D | |
| 91 | 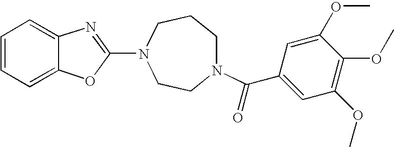 | | A |
| 92 | 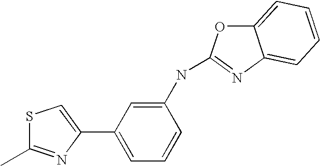 | | B |
| 93 | 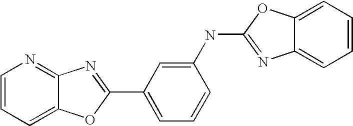 | D | |
| 94 | 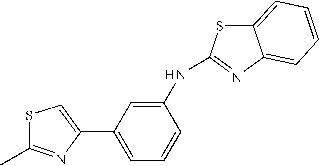 | D | |
| 95 | 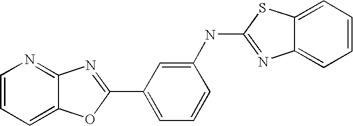 | NA | |
| 97 | 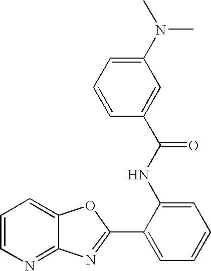 | A | |
| 98 | 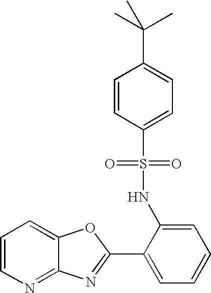 | NA | |
| 99 | 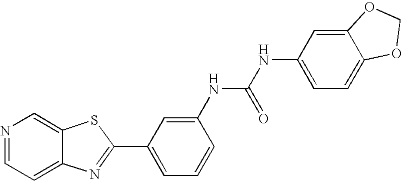 | A | |
| 100 | 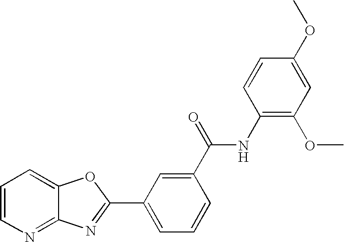 | A | |
| 101 | 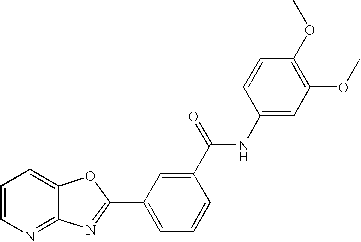 | C | |
| 102 | 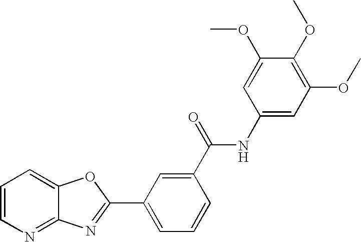 | A | |
| 103 | 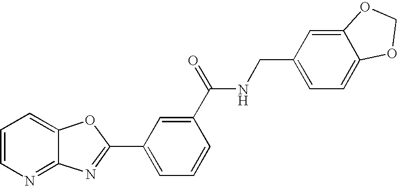 | NA | |
| 104 | 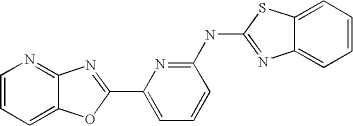 | A | |
| 105 | 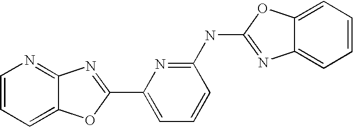 | A | |
| 133 | 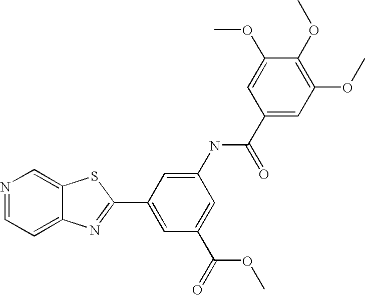 | NA | |
| 134 | 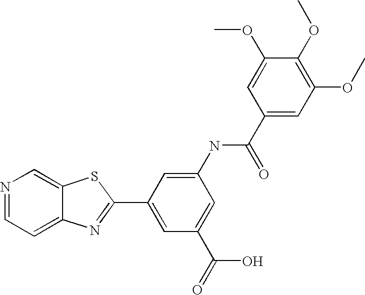 | NA | |
| 135 | 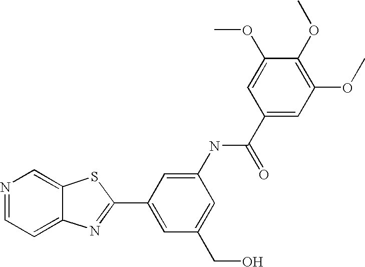 | A | |
| 106 | 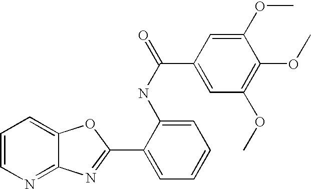 | A | |

In one embodiment, [symbol] -modulating compounds of the invention are represented by Structural Formula (I):

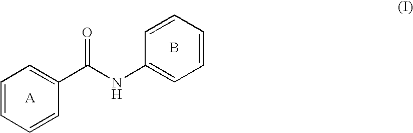

or a salt thereof, where:

Ring A is optionally substituted; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In certain embodiments, Ring B is substituted with at least a carboxy group.

In certain embodiments, Ring B is substituted with at least a substituted or unsubstituted arylcarboxamine, a substituted or unsubstituted aralkylcarboxamine or a polycyclic aryl group.

In certain embodiments, Ring B is substituted with at least a substituted or unsubstituted heteroaryl group or a substituted or unsubstituted heterocyclylcarbonylethenyl group;

In another embodiment, [symbol] modulating compounds of the invention are represented by Structural Formula (II):

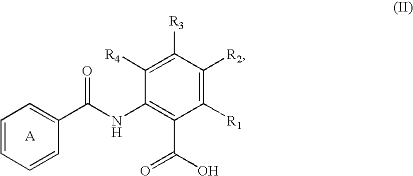

or a salt thereof, where:

Ring A is optionally substituted;

R₁, R₂, R₃ and R₄ are independently selected from the group consisting of —H, halogen, —OR₅, —CN, —CO₂R₅, —OCO₂R₅, —OCO₂R₅, —C(O)NR₅R₆, —OC(O)NR₅R₆, —C(O)R₅, —COR₅, —SR₅, —OSO₃H, —S(O)ₙR₅, —S(O)OR₅, —S(O)NR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In a further embodiment, [symbol] -modulating compounds of the invention are represented by Structural Formula (IIa):

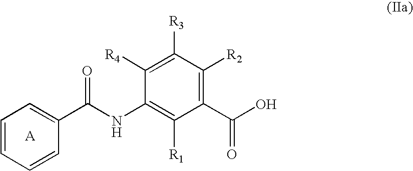

or a salt thereof, where:

Ring A is optionally substituted;

R₁, R₂, R₃ and R₄ are independently selected from the group consisting of —H, halogen, —OR₅, —CN, —CO₂R₅, —OCO₂R₅, —OCO₂R₅, —C(O)NR₅R₆, —OC(O)NR₅R₆, —C(O)R₅, —COR₅, —SR₅, —OSO₃H, —S(O)ₙR₅, —S(O)OR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another embodiment, [symbol] -modulating compounds of the invention are represented by Structural Formula (II):

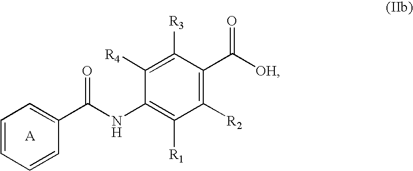

or a salt thereof, where:

Ring A is optionally substituted;

R₁, R₂, R₃ and R₄ are independently selected from the group consisting of —H, halogen, —OR₅, —CN, —CO₂R₅, —OCO₂R₅, —OCO₂R₅, —C(O)NR₅R₆, —OC(O)NR₅R₆, —C(O)R₅, —COR₅, —SR₅, —OSO₃H, —S(O)R₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ in Structural Formulas (II)-(IIb) are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention provides novel [symbol] modulating compounds of Formula (III):

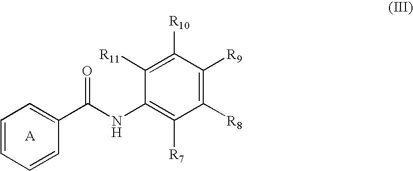

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCO₂R₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In certain such embodiments, R₈ is selected from thiazolopyridyl, imidazothiazolyl, benzoxazinonyl, or imidazopyridyl.

Particular examples of R₈, where

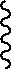

indicates attachment to the remainder of Structural Formula (III), include:

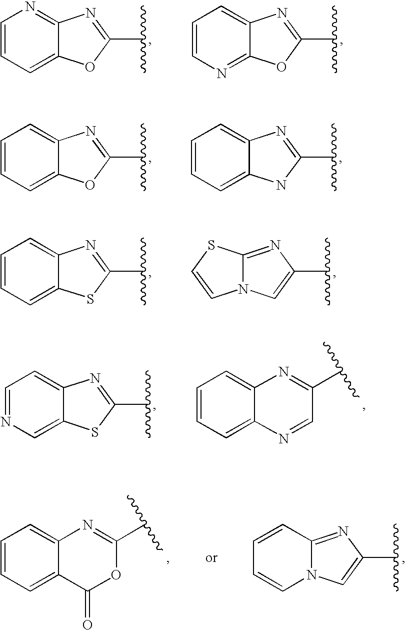

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

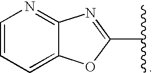

In certain embodiments, R₈ is

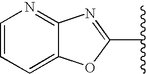

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

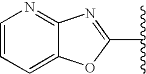

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

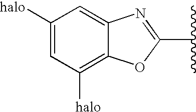

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

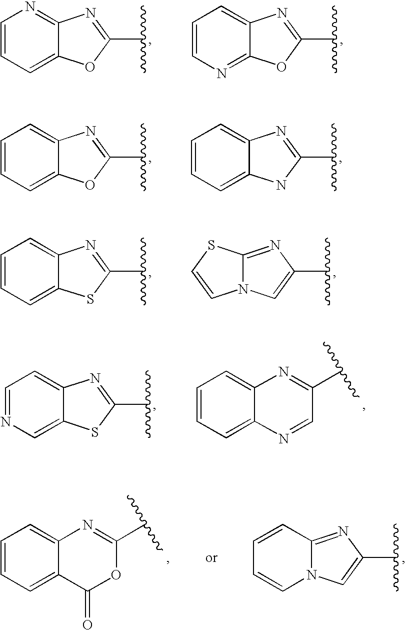

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

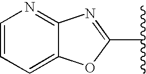

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

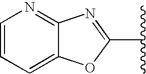

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In yet another aspect, the invention provides novel [symbol] -modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′ NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6-heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉, NHC(O)R₉, NH(COOR₉′), S(O)₂NR₉′R₉′, OC(O)R₉′; or R₉′.

In a preferred embodiment of the invention, each Ar, L, and Ar′ is independently an optionally substituted 5- to 7-membered monocyclic ring system or an optionally substituted 9- to 12-membered bicyclic ring system.

According to another preferred embodiment,

Ar is

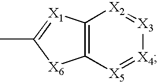

X₁, X₂, X₃, X₄, and X₅ are independently selected from CR₁′ and N; and

X₆ is selected from NR₁′, O, and S;

According to yet another preferred embodiment, X₁ and X₂ are N; X₃, X₄, and X₅ are CR₁′; and X₆ is O.

According to still yet another preferred embodiment, X₁ and X₃ are N; X₂, X₄, and Xs are CR₁′; and X₆ is O.

According to still yet another preferred embodiment, X₁ and X₄ are N; X₂, X₃, and X₅ are CR₁′; and X₆ is O.

According to still yet another preferred embodiment, X₁ and X₅ are N; X₂, X₃, and X₄ are CR₁′; and X₆ is O.

In another embodiment, the compounds of the formula above are those wherein J is NR₁′, K is absent, and M is C(O).

In yet another embodiment, the compounds of the formula above are those wherein J is absent, K is NR₁′, and M is C(O).

In a further embodiment, compounds of formula (IV) are those where when J is absent and K is NR₁′, M is not C(O) and when J is NR₁′ and K is absent, M is not C(O).

In a preferred embodiment, the compounds above are those wherein L is an optionally substituted 5- to 7-membered carbocyclic or heterocyclic aryl group.

In yet another preferred embodiment, the compounds are those wherein L is an optionally substituted phenylene, pyridinylene, imidazolylene, oxazolylene, or thiazolylene.

In a particularly preferred embodiment, L is an optionally substituted phenylene.

In another particularly preferred embodiment, L is an optionally substituted pyridinylene.

In an even more preferred embodiment, L is phenylene.

In another even more preferred embodiment, L is pyridinylene.

In either of these embodiments, Ar and J may be attached to L at the ortho-, meta-, or para-positions. Particularly preferred are those embodiments where attachment is at the meta-position.

In certain embodiments, L is not phenylene when Ar′ is phenyl. Examples of such embodiments include embodiments where L is an optionally substituted heterocyclic aryl group and Ar′ is an optionally substituted carbocyclic or heterocyclic aryl group, or wherein L is an optionally substituted carbocyclic or heterocyclic aryl group and Ar′ is an optionally substituted heterocyclic aryl group.

In yet another aspect, the invention provides novel [symbol] -modulating compounds of Formula (I) or a salt thereof, wherein

Ring A is substituted with at least one R₁′ group;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group;

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′; and

Ring B is substituted with at least one

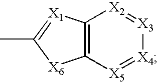

wherein

X₁, X₂, X₃, X₄, and X₅ are independently selected from CR₁′ and N; and

X₆ is selected from NR₁′, O, and S.

In a preferred embodiment, Ring B is phenyl or pyridinyl.

In a further aspect, the invention provides novel [symbol] -modulating compounds of Formula (IVa):

Het-L-Q-Ar′ (IVa)

or a salt thereof, wherein:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—,

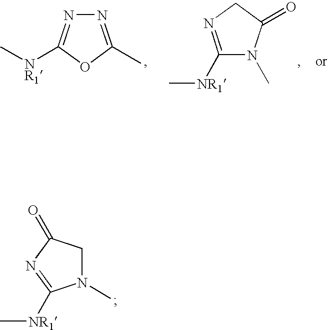

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In certain embodiments, when Het is a polycyclic heteroaryl, L is optionally substituted phenylene, and Ar′ is optionally substituted phenyl; then Q is not —NH—C(O)—.

In certain embodiments, Het and Q are attached to L in a 1-, 2- or 1-,3-configuration (e.g., when L is phenylene, Het and Q are attached in an ortho or a meta orientation). In certain embodiments where Het and Q are attached to L in a 1-,3-configuration, if Het is benzoxazolyl, L is pyridylene and Q is —NH—C(O)—NH, then Ar′ is not 3,4 dioxymethlyene phenyl; if Het is methyl thiazolyl, L is phenylene and Q is —NH—C(O)—, then Ar′ is not 3-dimethylamino phenyl; if Het is oxazolopyridyl, L is pyridylene and Q is —NH—C(O)—NH, then Ar′ is not 4-dimethylamino phenyl; if Het is oxazolopyridyl or benzoxazolyl and L is

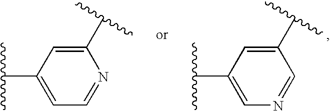

then Q is not —NH—(SO)₂—; and if Het is oxazolopyridyl, L is

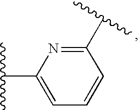

and Q is —NH—C(O)—, then Ar′ is not 3,4 dimethoxyphenyl or pyridyl.

When Het is substituted, it is typically substituted at up to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

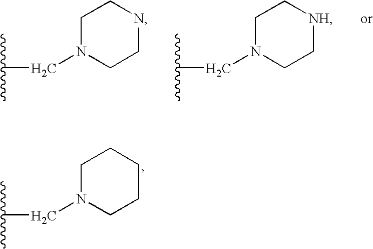

where each R₁₂ is independently selected from optionally substituted C₁-C₃ straight or branched alkyl.

In certain embodiments, Het is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In other embodiments, Het comprises one ring N heteroatom and 1 to 2 additional ring heteroatoms independently selected from N, O or S, such as thiazolyl, triazolyl, oxadiazolyl, thiazolopyridyl, imidazothiazolyl, benzoxazinonyl, or imidazopyridyl.

Particular examples of Het include:

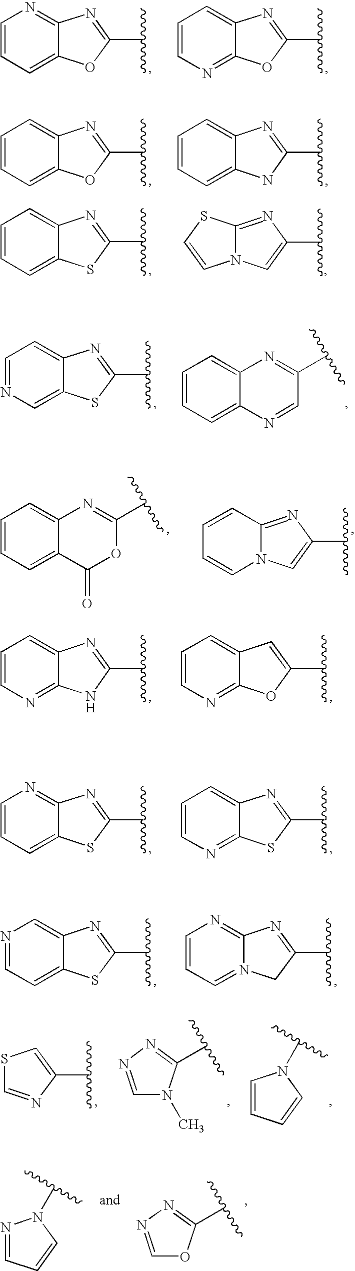

where up to 2 ring carbons not immediately adjacent to the indicated attachment point are independently substituted with optionally substituted C₁-C₃ straight or branched alkyl, phenyl, halo, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

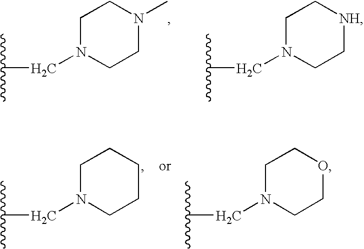

wherein each R₁₂ is independently selected from optionally substituted C₁-C₁ straight or branched alkyl.

In certain embodiments, L is selected from

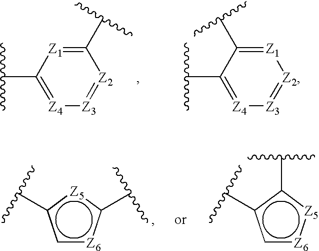

wherein: each of Z₁, Z₂, Z₃ and Z₄ is independently selected from CH or N, wherein not more than three of said Z₁, Z₂, Z₃ or Z₄ is N;

each of Z₅ and Z₆ is independently selected from C, N, O or S, provided that at least one of Z₅ and Z₆ is N; and

L is optionally substituted at 1 to 2 carbon atoms with a substituent independently selected from R₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

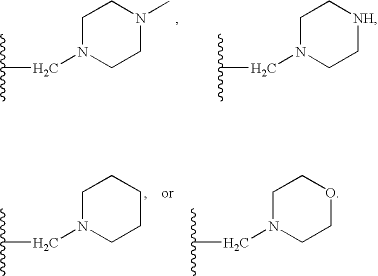

In preferred embodiments, L is selected from phenylene or pyridylene, such as unsubstituted phenylene or phenylene substituted with a single substituent selected from C(O)OCH₃, C(O)OH, CH₂OH, N(CH₃)₂, or CH₂N(CH₃)₂, or unsubstituted pyridylene.

In certain embodiments, Q is selected from —NH—C(O)—, —NH—S(O)₂—, —NH—C(O)—NH—, —C(O)—NH—, —CH₂—, —N(CH₃)—C(O)—NH—, —NH—C(O)—N(CH₃)—, or —NH—S(O)₂—NH—, particularly —NH—C(O)—, —C(O)—NH—, —NH—, —NH—C(O)—NH, or —NH—S(O)₂—.

In certain embodiments, Ar′ is selected from optionally substituted phenyl, benzothiazolyl, or benzoxazolyl. When Ar′ is phenyl, typical optional substituents are 1 to 3 substituents independently selected from halo, (optionally substituted C₁-C₃ straight or branched alkyl), O-(optionally substituted C₁-C₃ straight or branched alkyl), S-(optionally substituted C₁-C₃ straight or branched alkyl), N(CH₃)₂ or optionally substituted heterocyclyl, or wherein two substituents on adjacent ring atoms are taken together to form a dioxymethylene.

In certain embodiments, Het is selected from

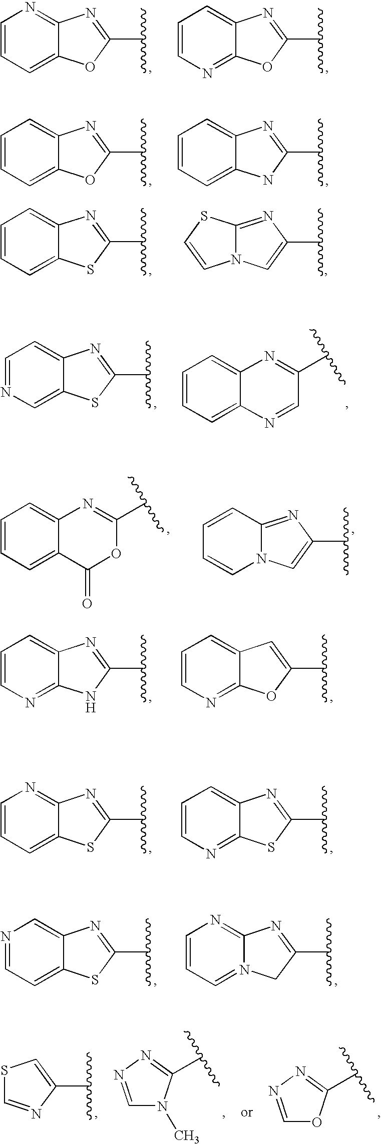

and wherein up to 2 ring carbons not immediately adjacent to the indicated attachment point are independently substituted with optionally substituted C₁-C₃ straight or branched alkyl, phenyl or halo;

L is selected from unsubstituted phenylene, phenylene substituted with a single substituent selected from C(O)OCH₃, C(O)OH, CH₂OH, N(CH₃)₂, or CH₂N(CH₃)₂, or unsubstituted pyridylene;

Q is selected from —NH—C(O)—, —C(O)—NH—, —NH—, —NH—C(O)—NH, or —NH—S(O)₂—; and

Ar′ is selected from optionally substituted phenyl, benzothiazolyl, or benzoxazolyl, wherein said phenyl is optionally substituted with 1 to 3 substituents independently selected from chloro, methyl, O-methyl, S-methyl, N(CH₃)₂, morpholino, or 3,4 dioxymethylene.

In certain embodiments, Q is selected from —NH—C(O)—, —C(O)—NH—, —NH— or —NH—C(O)—NH.

In certain embodiments, the substituents on Ar′ are selected from chloro, methyl, O-methyl, S-methyl or N(CH₃)₂. In certain embodiments, the only substituent on Ar′ is an O-methyl group, particularly an O-methyl group ortho or meta to Q. In certain embodiments, when there are two or more O-methyl groups or Ar′, at least one is ortho or meta to Q.

In certain embodiments, L is pyridyl and Het and Q are at the 1,3- or 2,4-position with respect to the pyridyl nitrogen atom. In certain such embodiments, Q is —NH—S(O)₂—.

In certain embodiments where L is further substituted, the substituent is typically meta to both Het and Q.

In certain embodiments, Q is —NH— and Het is thiazolyl or oxazolopyridyl.

In certain embodiments, Q is —NH— and Ar is benzothiazolyl or benzoxazolyl.

In certain embodiments, L is

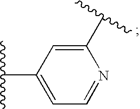

and Q is —NH—(SO)₂—. In certain such embodiments, Het is oxazolopyridyl. When L, Q and optionally Het have these values, Ar′ is advantageously naphthyl or phenyl, where Ar′ is optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments, L is

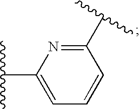

and Q is —NH—C(O)—. In certain such embodiments, Het is oxazolopyridyl. When L, (and optionally Het have these values, Ar′ is advantageously pyridyl or phenyl optionally substituted with 1 to 3 substituents independently selected from CN, halo, (C1-C3 straight or branched alkyl), O—(C1-C3 straight or branched alkyl), N(C1-C3 straight or branched alkyl)₂, or a 5 to 6-membered heterocycle.

In certain embodiments,

, Het comprises one N heteroatom and 1 to 2 additional heteroatoms independently selected from N, O or S;

L is

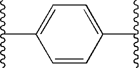

and is optionally substituted;

Q is —NH—C(O)—; and

Ar′ is phenyl substituted with 1 to 3 substituents independently selected from CN, halo, C₁-C₃ straight or branched alkyl, O—(C₁-C₃ straight or branched alkyl), N(C₁-C₃ straight or branched alkyl)₂, or a 5 to 6-membered heterocycle, wherein when R₈ is unsubstituted

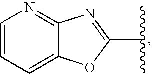

then ring A is:
a) not simultaneously substituted at the 2- and 6-positions with O—(C₁-C₃ straight or branched alkyl);
b) not simultaneously substituted at the 2-position with C₁-C₃ straight or branched alkyl or O—(C₁-C₃ straight or branched alkyl) and at the 3-position with O—(C₁-C₃ straight or branched alkyl);
c) not substituted at the 4-position with O—(C₁-C₃ straight or branched alkyl) unless simultaneously substituted at the 3-position with halo or O—(C₁-C₃ straight or branched alkyl) and unsubstituted at all other positions;
   not substituted at the 4-position with N(C₁-C₃ straight or branched alkyl)₂, or said 5 to 6-membered heterocycle. In certain such embodiments, L is unsubstituted and/or Het is oxazolopyridyl.

In yet another aspect, the invention provides novel [symbol] modulating compounds of Formula (V):

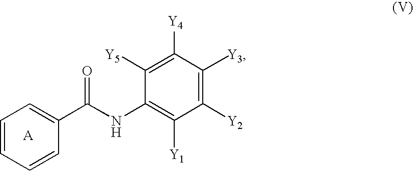

or a salt thereof, wherein:

Ring A is optionally substituted with at least one R₁′ group;

Y₁, Y₂, Y₃, Y₄, and Y₅ are independently R₁′;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆₁, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉, OC(O)R₉′; or R₉′.

In a preferred embodiment of the above compound,

either Y₂ or Y₃ is

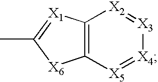

X₁, X₂, X₃, X₄, and X₅ are independently selected from CR₁′ and N; and

X₆ is selected from NR₁′, O, and S.

According to an even more preferred embodiment, X₁ and X₂ are N; X₃, X₄, and X₅ are CR₁′; and X₆ is O.

According to another even more preferred embodiment, X₁ and X₃ are N; X₂, X₄, and X₅ are CR₁′; and X₆ is O.

According to another even more preferred embodiment, X₁ and X₄ are N; X₂, X₃, and X₅ are CR₁′; and X₆ is O.

According to another even more preferred embodiment, X₁ and X₅ are N; X₂, X₃, and X₄ are CR₁′; and X₆ is O.

In another aspect, the invention provides [symbol] -modulating compounds of Structural Formula (VII):

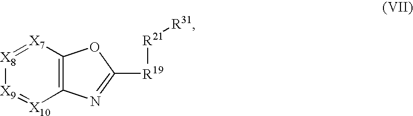

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

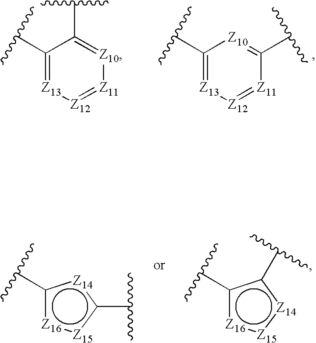

wherein:
each Z₁₀, Z₁₁, Z₁₂ and Z₁₃ is independently selected from N CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

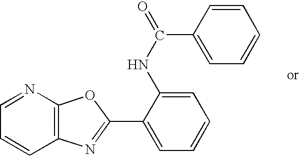

that when R¹⁹ is

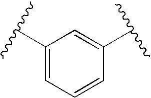

and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

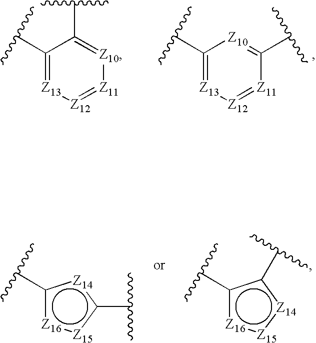

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR₂, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

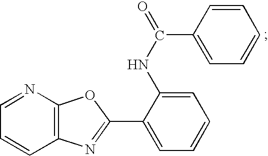

and

when X₈ and X₉ are each independently selected from CR²⁰ or CR₁′, R¹⁹ is

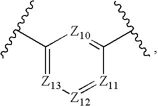

and each of Z₁₀, Z₁₁, Z₁₂ and Z₁₃ is independently selected from CR²⁰, or CR₁′, then:

a) at least one of X₈ and X₉ is not CH; or

b) at least one of Z₁₀, Z₁₁, Z₁₂ and Z₁₃ is CR²⁰, wherein R²⁰ is a solubilizing group.

In certain embodiments, when Z₁₂ is CR²⁰ and R²⁰ is a solubilizing group, the solubilizing group is not —C(O)OCH₂ CH₃, —COOH,

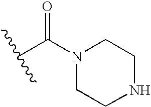

or

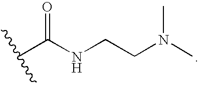

In certain embodiments, when X₈ and X₉ are each independently selected from CR²⁰ or CR₁′, R¹⁹ is

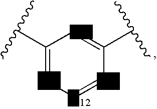

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁ and Z₁₃ is CR²⁰, wherein R²⁰ is a solubilizing group.

In certain embodiments, when R¹⁹ is

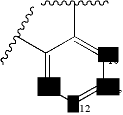

and each of Z₁₀, Z₁₁, Z₁₂ and Z₁₃ is CR²⁰, or CR₁′; X₈ and X₉ are CR²⁰ or CR₁′; R²¹ is —NHC(O)—; and R³¹ is optionally substituted phenyl, then R³¹ is a substituted phenyl, at least one R₁′ in a CR₁′ moiety is optionally substituted C₁-C₃ straight or branched alkyl, or at least one R²⁰ in a CR²⁰ is a solubilizing group, or a combination thereof.

In certain embodiments, R¹⁹ is selected from phenyl, pyridyl, thienyl or furyl.

In certain embodiments, R¹⁹ is

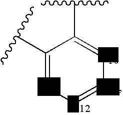

wherein each of Z₁₀, Z₁₁, Z₁₂ and Z₁₃ is s independently selected from CR²⁰ or CR₁′; and

R²¹ is —NH—C(O)—; and

R³¹ is a substituted phenyl.

In certain such embodiments, when X₉ is N, R³¹ is not 2,4 dimethoxyphenyl and/or when X₁₀ is N, R³¹ is not halo substituted phenyl; 3,4-dioxoethylenephenyl; or 3,5-dimethoxyphenyl.

In preferred embodiments, R³¹ is optionally substituted with 1 to 3 substituents independently selected from —OCH₃, —CH₃, —N(CH₃)₂, pyrazinoxy or a solubilizing group. Suitable examples of R³¹ include 3-methoxy-4-((4-methylpiperazin-1-yl)methyl)phenyl, 3-methoxy-4-morpholinomethylphenyl, 3-methoxy-4-diaminomethylphenyl, 3-methoxy-4-((pyrrolidin-1-yl)methyl)phenyl, 3,4-dimethoxyphenyl, 3,5-dimethoxyphenyl, 2,3,4-trimethoxyphenyl, 3,4,5-trimethoxyphenyl, 2-dimethylaminophenyl, 3-dimethylaminophenyl, 4-dimethylaminophenyl, or 3,5-dimethylphenyl.

In certain embodiments, R¹⁹ is selected from

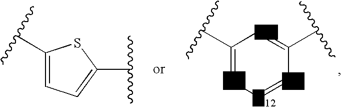

wherein one of Z₁₀, Z₁₁, Z₁₂, and Z₁₃ is N and the others are independently selected from CR²⁰ or CR₁′;

R²¹ is selected from —NH—, —NH—C(O)—, —NH—C(O)—NH, —NH—C(S)—NH— or —NH—S(O)₂—; and

R³¹ is selected from an optionally substituted phenyl, an optionally substituted naphthyl, or an optionally substituted heteroaryl.

In certain such embodiments,
a) when R²¹ is —NH—S(O)₂—, either:
   i) Z₁₀ is N; or
   ii) Z₁₁ is N and R³¹ is halophenyl or 2-methoxy-5-methylphenyl;
b) when R¹⁹ is

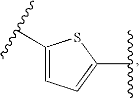

R³¹ is not 4-dimethylaminophenyl, 2,3,4-trimethoxyphenyl; or 3,5 dimethoxyphenyl; and/or
c) when R²¹ is —NH—C(O)—NH— and Z₁₀ is N, R³¹ is not 4-dimethylaminophenyl.

In certain such embodiments, R³¹ is selected from optionally substituted phenyl, benzothiazolyl, or benzoxazolyl.

In yet another embodiment, the invention provides [symbol] -modulating compounds of Structural Formula (VIII):

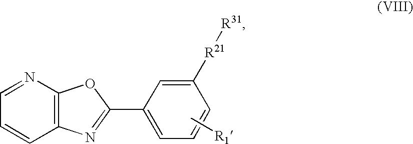

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′-S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

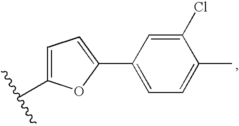

1-methoxynaphthyl, 2-methoxynaphthyl, or unsubstituted 2-thienyl;

when R₁′ is methyl, and R²¹ is —NH—C(O)—CH═CH—, R³¹ is not

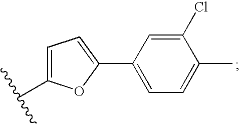

when R₁′ is methyl, and R²¹ is —NH—C(O)—CH—O—, R³¹ is not unsubstituted naphthyl, 2-methoxy, 4-nitrophenyl, 4-chloro-2-methylphenyl, or 4-t-butylphenyl; and

when R²¹ is —NH—C(O)—, R³¹ is not optionally substituted phenyl.

In certain embodiments, R²¹ is —NH—C(O)—; and R³¹ is phenyl optionally substituted with 1 to 3 substituents independently selected from —OCH₃, —CH₃, —N(CH₃)₂, or a solubilizing group.

In certain such embodiments, R²¹ is —NH—C(O)— and R³¹ is selected from unsubstituted phenyl, 2-methoxyphenyl, 3-methoxyphenyl, 2,3,4-trimethoxyphenyl, 3,4,5-trimethoxyphenyl, 2,4-dimethoxyphenyl, 3,5-dimethoxyphenyl, 2-methyl-3-methoxyphenyl, 2-morpholinophenyl, 2-methoxy-4-methylphenyl, 2-dimethylaminophenyl, 4-dimethylaminophenyl, or

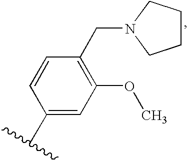

particularly phenyl; 2-methoxyphenyl; 3-methoxyphenyl; 2,3,4-trimethoxyphenyl; 3,4,5-trimethoxyphenyl; 2,4-dimethoxyphenyl; 3,5-dimethoxyphenyl; 2-methyl-3-methoxyphenyl; 2-morpholinophenyl; 2-methoxy-4-methylphenyl; 2-dimethylaminophenyl; or 4-dimethylaminophenyl.

In a further embodiment, the invention provides [symbol] -modulating compounds of Structural Formula (IX):

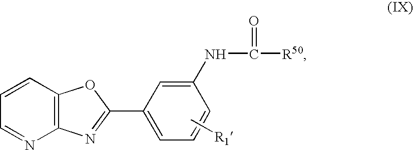

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

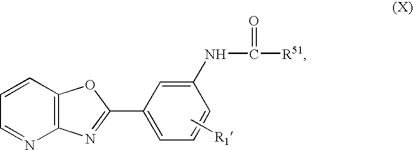

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In certain embodiments, R⁵¹ is selected from pyrazolyl, thiazolyl, oxazolyl, pyrimidinyl, furyl, thienyl, pyridyl, isoxazolyl, indolyl, benzopyrazolyl, benzothiazolyl, benzoxazolyl, quinoxalinyl, benzofuranyl, benzothienyl, uinolinyl, benzoisoxazolyl, benzotriazinyl, triazinyl, naphthyl, or

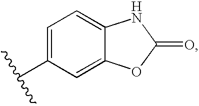

and wherein R⁵¹ is optionally substituted. In certain such embodiments, R⁵¹ is selected from pyrazolyl, thiazolyl, oxazolyl, pyrimidinyl, indolyl, pyrazinyl, triazinyl, or

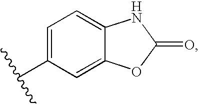

and R⁵¹ is optionally substituted.

In another aspect, the invention provides [symbol] -modulating compounds of Structural Formula (XI):

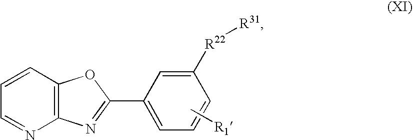

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In certain embodiments, R²² is selected from —C(O)—NH—, —NH—, or —C(O)—NH—CH₃.

In certain embodiments, such as when R²² is selected from —C(O)—NH—, —NH—, or —C(O)—NH—CH₃, R³¹ is selected from optionally substituted phenyl, benzothiazolyl, quinoxalinyl, or benzoxazolyl.

In yet another aspect, the invention provides [symbol] modulating compounds of Structural Formula (XII):

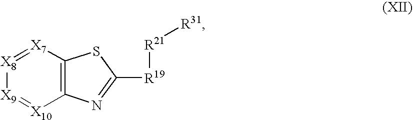

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

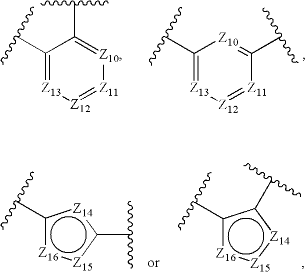

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄; Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(—NR₁′—NR₁′—, —C(O)—NR₁′—, —C(O)—NR₁′—S(O)₂—, —NR₁′—, —CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—-C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that when R¹⁹ is

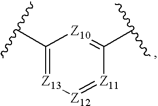

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

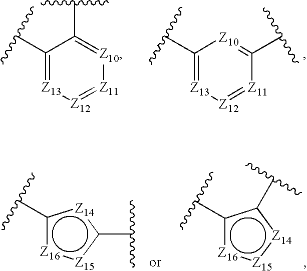

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

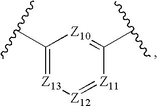

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In certain embodiments, R²¹ is —NH—C(O)— and R¹⁹ selected from:

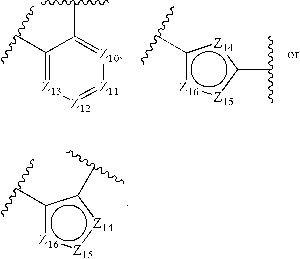

In certain embodiments, R¹⁹ is selected from optionally substituted phenyl, optionally substituted pyridyl, optionally substituted thienyl or optionally substituted furyl.

In certain embodiments, R¹⁹ is

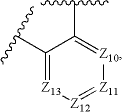

wherein each of Z₁₀, Z₁₁, Z₁₂ and Z₁₃ is independently selected from CR²⁰ or CR₁′; and

R²¹ is selected from —NH—C(O)—, —NH—C(O)—CH(CH₃)—O—, —NH—C(O)—CH₂—O—, or —NH—S(O)₂—CH₂—CH₂—; and

R³¹ is selected from an optionally substituted aryl, or an optionally substituted heteroaryl.

In certain such embodiments, R³¹ is optionally substituted with 1 to 3 substituents independently selected from —OCH₃, —CH₃, —N(CH₃)₂, phenyl, phenoxy, 3,4-dioxymethylene, fluoro, or another solubilizing group. Suitable examples of R³¹ include unsubstituted quinolinyl, 2,4-dimethoxyphenyl, 3,4-dimethoxyphenyl, 3,5-dimethoxyphenyl, 3,4,5-trimethoxyphenyl, 2,3,4-trimethoxyphenyl, 2-dimethylaminophenyl, 3-dimethylaminophenyl, 4-dimethylaminophenyl, 3,5-dimethylphenyl, 3,5-difluorophenyl, 3-trifluoromethoxyphenyl, unsubstituted quinoxalinyl, unsubstituted benzopyrimidinyl,

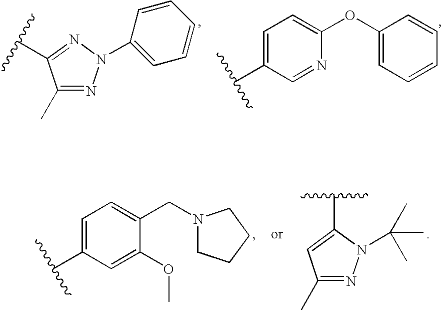

In certain such embodiments, R³¹ is not phenyl-substituted furyl.

In certain embodiments, R¹⁹ is selected from

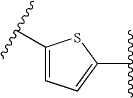

or

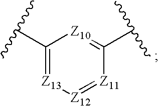

each of Z₁₀, Z₁₁, Z₁₂ and Z₁₃ is independently selected from CR²⁰, or CR₁′;

R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

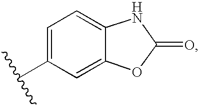

and R³¹ is optionally substituted (e.g., optionally substituted with up to three substituents independently selected from —OCH₃, —CH₃, —N(CH₃)₂, —O-phenyl, or another solubilizing group). Suitable examples of R³¹ include unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

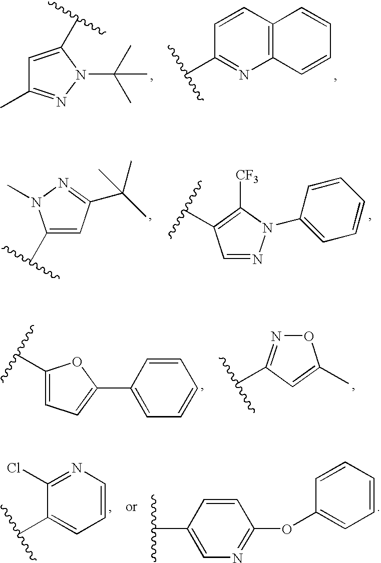

In certain embodiments, one or more of the following conditions applies:

when X₈ is N, R²¹ is —NH—C(S)—NH—, and R¹⁹ is phenyl, R³¹ is not 2-methoxy-5-nitrophenyl, 2-S-methylphenyl or 2-acetylphenyl;

when X₈ is N, R²¹ is —NH—S(O)₂—, and R¹⁹ is phenyl, R³¹ is not thiadiazole-substituted thienyl or 4-methylsulfonylphenyl;

when X₈ is N, R²¹ is —NH—CO—, and R¹⁹ is phenyl, R³¹ is not 2,4-difluorophenyl, pyridyl-substituted thienyl, 3,4-dichlorophenyl, 4-t-butylphenyl, or 3-benzyloxyphenyl;

when X₉ is N, R²¹ is —NH—C(O)— and R¹⁹ is

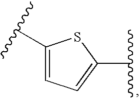

R³¹ is not 2,3,4-trimethoxyphenyl or 3,5-dimethoxyphenyl; and

when X₉ is N, R²¹ is —NH—C(O)— and R¹⁹ is phenyl, R³¹ is not 3,5-dimethoxyphenyl.

In a further embodiment, the invention provides compounds of Structural Formula (XIII):

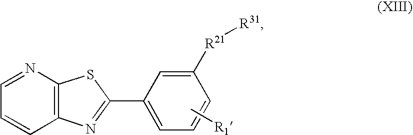

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl; with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In certain embodiments, R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from a monocyclic or bicyclic aryl or a monocyclic or bicyclic heteroaryl, and comprises a solubilizing group substituent.

In certain embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

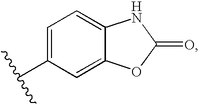

and R³¹ is optionally substituted.

In certain embodiments, R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, particularly when R²¹ is —NH—C(O)—, R³¹ is selected from R³¹ is selected from unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

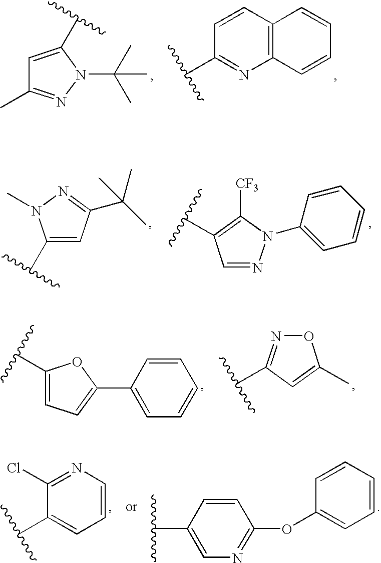

In one aspect, the invention provides [symbol] -modulating compounds of Structural Formula (XIV):

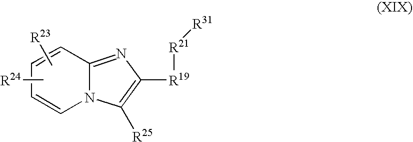

or a salt thereof, wherein:

each of R² and R²⁴ is independently selected from H, —CH₃ or a solubilizing group;

R²⁵ is selected from H or a solubilizing group; and

R¹⁹ is selected from:

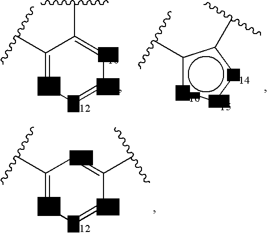

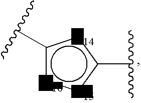

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

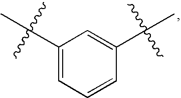

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In certain embodiments, each of R²³ and R²⁴ is independently selected from H, —CH₃ or a solubilizing group;

R²⁵ is selected from H, or a solubilizing group; and

R¹⁹ is selected from:

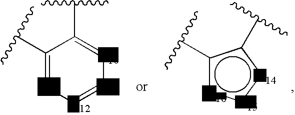

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—; —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, R³¹ is not 2,4-dimethoxyphenyl.

Typically, R²⁵ is selected from H, —CH₂—N(CH₃)₂, or

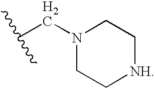

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In certain embodiments, each of R²³ and R²⁴ is independently selected from H, —CH₃ or a solubilizing group;

R²⁵ is selected from H, or a solubilizing group; and

R¹⁹ is selected from:

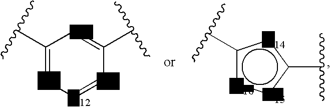

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ is N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′, —C(O)—NR₁′—, —C(O)—NR₁′—S(O)₂, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is phenyl, at least one of R²³, R²⁴, or R²⁵ is a solubilizing group and wherein said compound is not 2-chloro-N-[3-[3-(cyclohexylamino)imidazo[1,2-a]pyridin-2-yl]phenyl]-4-nitrobenzamide.

Typically, R²⁵ is selected from H, —CH₂—N(CH₃)₂, or

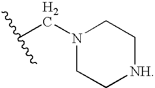

Typically, R²³ and R²⁴ are H.

Typically, R¹⁹ is selected from phenyl, pyridyl, thienyl or furyl, particularly optionally substituted phenyl. Preferably, a phenyl is optionally substituted with:

b) up to three —O—CH₃ groups; or

b) one —N(CH₃)₂ group.

In another aspect, the invention provides [symbol] -modulating compounds of Structural Formula (XV):

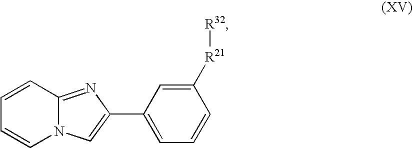

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂O—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

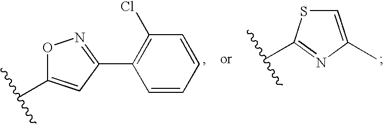

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

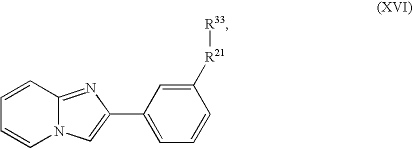

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, —NR₁′—C(═N—CN)—NR₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In certain embodiments, R²¹ is selected from —NR²²—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁—, —NR₁′—C(═N—CN)—NR₁′—, or —NR₁′—C(O)—CR₁′R₁′—; and

each R₁′ is independently selected from H or optionally substituted C₁-C₃ straight or branched alkyl;

R²² is an optionally substituted C₁-C₃ straight or branched alkyl; and

R³³ is phenyl comprising a solubilizing group substituent, wherein: when R²¹ is —NH—S(O)₂ said phenyl comprises an additional substituent.

In certain embodiments, R²¹ is selected from —NR²²—C(O)—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, or —NR₁′—C(═N—CN)—NR₁′—,

each R₁′ is independently selected from H or optionally substituted C₁-C₃ straight or branched alkyl; and

R²² is an optionally substituted C₁-C₃ straight or branched alkyl.

In certain embodiments, R³³ is optionally substituted on up to three carbon atoms with a substituent independently selected from —O—CH₃—CH₃, —N(CH₂, —S(CH₃), or CN; or substituted on adjacent carbon atoms with

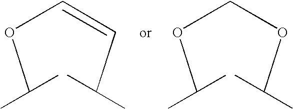

bridging said adjacent carbon atoms.

In a further embodiment, the invention provides: [symbol] -modulating compounds of Structural Formula (XVII):

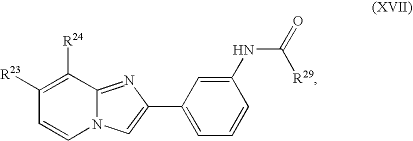

or a salt thereot wherein:

each of R²³ and R²⁴ is independently selected from H or —CH₃, wherein at least one of R²⁷ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position,

wherein R²⁹ is optionally additionally substituted with a solubilizing group.

In certain embodiments, R²⁹ is phenyl substituted with:

a) three —O—CH₃ groups located at the 2, 3 and 4 positions; or

b) one —N(CH₃)₂ group.

In one aspect, the invention provides [symbol] -modulating compounds of Structural Formula (XVIII):

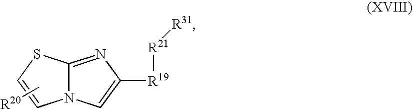

or a salt thereof, wherein

R¹⁹ is selected from:

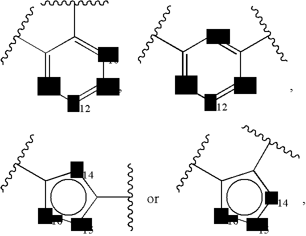

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

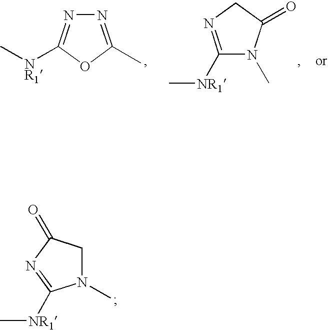

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl with the proviso that when R¹⁹ is

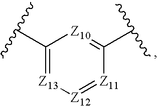

Z₁₀, Z₁₁, Z₁₂ and Z₁₃ are each CH, R²⁰ is H, and R²¹ is —NHC(O)—, R³¹ is not an optionally substituted phenyl.

In certain embodiments, R¹⁹ is selected from:

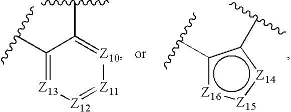

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

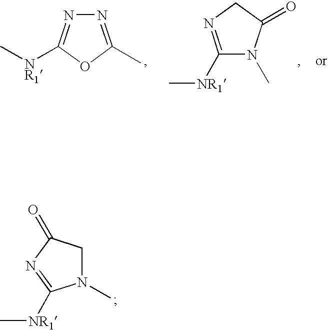

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, compounds of Structural Formula (XVIII) have the formula:

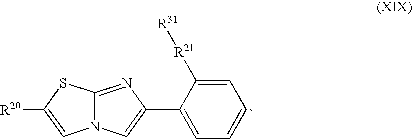

or a salt thereof, wherein

R²⁰ is selected from H or a solubilizing group;

R²¹ is selected from —NH—C(O)—, or —NH—C(O)—CH₂—; and

R³¹ is selected from an optionally substituted monocyclic or bicyclic aryl, or an optionally substituted monocyclic or bicyclic heteroaryl.

Typically, R¹⁹ in compounds of Structural Formula (XVIII) is selected from phenyl, pyridyl, thienyl or furyl, particularly optionally substituted phenyl.

Typically, R²⁰ is selected from H, —CH₂—N(CH₃)₂,

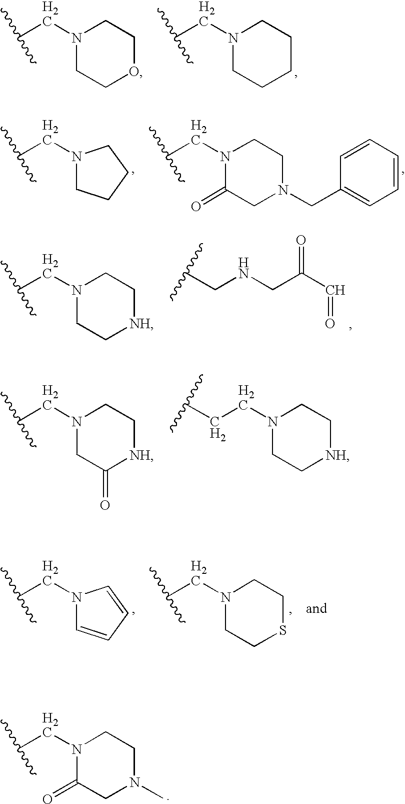

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In certain such embodiments, when R²¹ is —NR₁′—C(O)—, R³¹ is not 4-cyanophenyl or

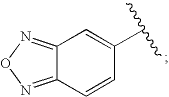

and/or when R²¹ is —NR₁′—S(O)₂—, R³¹ is not 4-methoxyphenyl or 4-t-butylphenyl.

In certain such embodiments, when R¹⁹ is

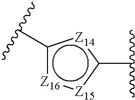

or

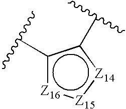

and R²¹ is —NR₁′—C(O)—, R³¹ is not 4-cyanophenyl or

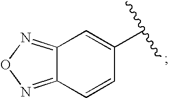

and/or when R¹⁹ is

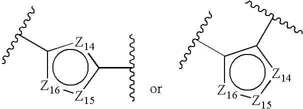

and R²¹ is —NR₁′—S(O)₂—, R³¹ is not 4-methoxyphenyl or 4-t-butylphenyl.

In another aspect, the invention provides [symbol] -modulating compounds of Structural Formula (XX):

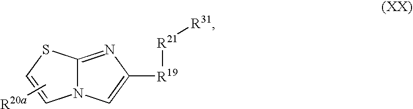

or a salt thereof, wherein

R¹⁹ is selected from:

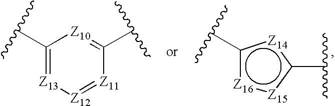

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

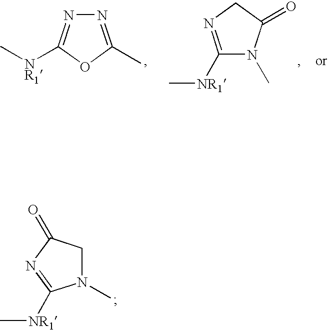

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

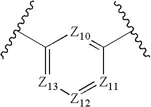

and Z₁₀, Z₁₁, Z₁₂ and Z₁₃ are each CH, R^{20a} is a solubilizing group.

Typically, R¹⁹ in compounds of Structural Formula (XX) is selected from phenyl, pyridyl, thienyl or furyl, particularly optionally substituted phenyl.

Typically, R^{20a} is selected from H, —CH₂—N(CH₃)₂,

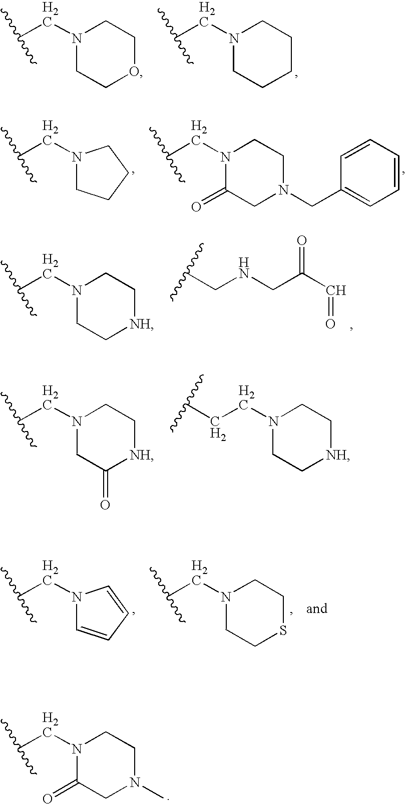

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In yet another aspect, the invention provides [symbol] -modulating compounds of Structural Formula (XXI):

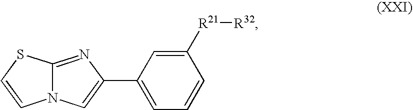

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

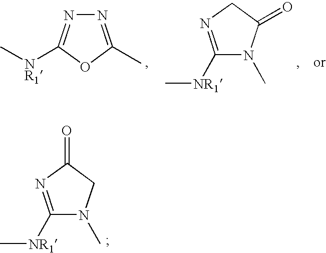

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In certain embodiments, R³² is selected from pyrrolyl, pyrazolyl, pyrazinyl, furyl, pyridyl, pyrimidinyl, or thienyl, and R³² is optionally substituted and is optionally benzofused.

In certain embodiments, R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

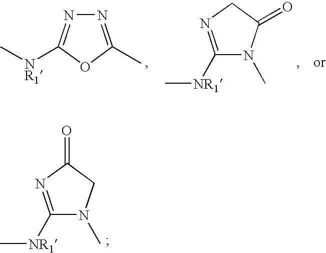

wherein
each R₁′ is independently selected from H or optionally substituted C₁-C₃ straight or branched alkyl; and

R³² is selected from benzofuryl, methylfuryl, benzothienyl, pyridyl, pyrazinyl, pyrimidinyl, pyrazolyl, wherein said methylfuryl, pyridyl, pyrazinyl, pyrimidinyl or pyrazolyl is optionally benzofused and wherein R³² is optionally substituted or further substituted.

In a further aspect, the invention provides [symbol] -modulating compounds of Structural Formula (XXII):

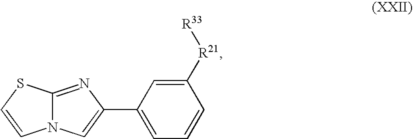

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—C(O)—CR₁′R₁′—O—, —NR₁′—S(O)₂—CR₁′R₁′—, —NR₁′—S(O)₂—CR₁′R₁′—CR₁′R₁′—,

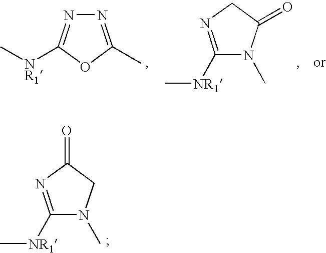

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

Preferably, R²¹ is selected from —NH—C(O)— or —NH—C(O)—CH₂—.

In one aspect, the invention provides [symbol] modulating compounds of Structural Formula (XXII):

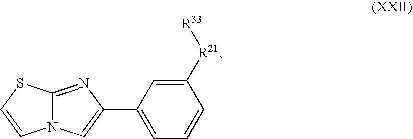

or a salt thereof where

R²¹ is selected from —NH—C(O)—, or —NH—C(O)—CH₂—; and

R³³ is phenyl substituted with

e) one —N(CH₃)₂ group;

f) one CN group at the 3 position;

g) one —S(CH₃) group; or

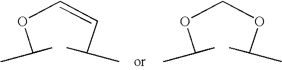

bridging the 3 and 4 positions.

In another aspect, the invention provides [symbol] -modulating compounds of Structural Formula (XXIII):

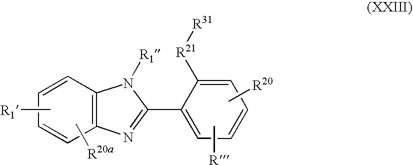

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl; with the provisos that:
when R²¹ is —NH—C(O)—, R³¹ is not is not 3,5-dinitrophenyl, 4-butoxyphenyl

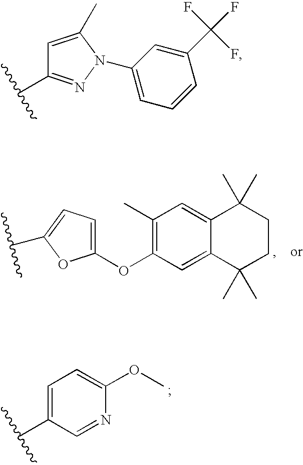

when R²¹ is —NH—C(O)— and each of R²⁰, R^{20a}, R₁′, R₁″ and R₁′″ is hydrogen, R¹³ is not

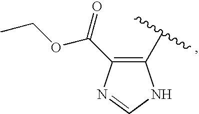

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;
when R²¹ is —NH—C(O)—, R₁″ is methyl; and each of R²⁰, R^{20a}, R₁′ and R₁′″ is hydrogen, R³¹ is not 2-methylaminophenyl,

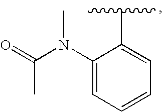

or

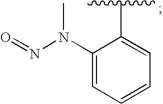

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;
when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

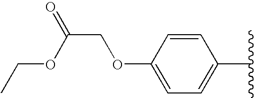

or

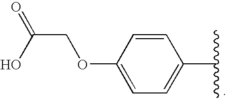

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In a particular aspect, the invention provides [symbol] -modulating compounds of Structural Formula (XXIII):

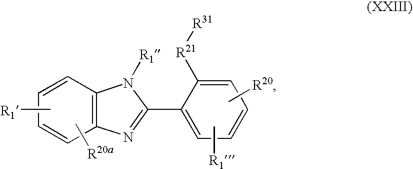

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In certain embodiments. R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In yet another aspect, the invention provides [symbol] -modulating compounds of Structural Formula (XXIV):

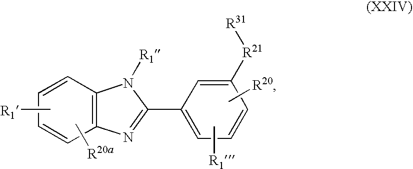

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—CH₂—, R³¹ is not 2-methylphenyl, or 3,4-dimethoxyphenyl;
when R²¹ is —NH—C(O)—CH═CH—, R³¹ is not 2-chlorophenyl;
when R²¹ is —NH—C(O)—NH—, R³¹ is not unsubstituted benzimidazolyl;
when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;
when R²¹ is —NH—S(O)₂—, each of R₁′ and R₁′″ is methyl or hydrogen, and each of R²⁰, R^{20a}, and R₁″ is hydrogen, R₃₁ is not 4-nitrophenyl;
when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;
when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

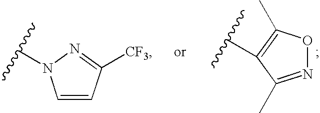

when R²¹ is —NH—C(O)—CH₂—, R₁′ is methyl, and each of R²⁰, R^{20a}, R₁″, and R₁′″ is hydrogen, R³¹ is not unsubstituted phenyl;
when R²¹ is —NH—C(O)—CH═CH, R₁″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;
when R²¹ is —NH—C(O)—CH(CH₂CH₃)—, and each of R²⁰, R^{20a}, R₁′, R₁″, and R₁′″ is hydrogen, R³¹ is not unsubstituted phenyl;
when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides: [symbol] -modulating compounds of Structural Formula (XXIV):

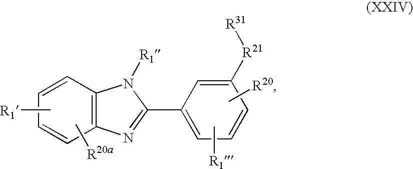

or a salt thereof, wherein:
each R²⁰ and R^{20a} is independently selected from H or a solubilizing group and at least one of R²⁰ and R^{20a} is a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and
R³¹ is selected from an optionally substituted monocyclic or bicyclic aryl, or an optionally substituted monocyclic or bicyclic heteroaryl.

In certain embodiments; when R²¹ is —NH—C(O)—CH₂—, R³¹ is not 2-methylphenyl; or 3,4-dimethoxyphenyl; when R²¹ is —NH—C(O)—CH═CH—, R³¹ is not 2-chlorophenyl; and/or when R²¹ is —NH—C(O)—NH—, R³¹ is not unsubstituted benzimidazolyl.

In a further aspect, the invention provides [symbol] -modulating compounds of Structural Formula (XXV):

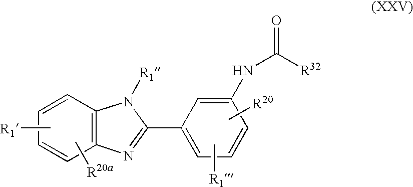

or a salt thereof, wherein:
each R²⁰ and R^{20a} is independently selected from H or a solubilizing group, wherein at least one of R²⁰ and R^{20a} is a solubilizing group;
each R₁′, R₁″ and R₁′″ is independently selected from H or optionally substituted C₁-C₃ straight or branched alkyl; and
R³² is an optionally substituted phenyl.

In certain embodiments, R³² is selected from 3,4-dimethoxyphenyl, 2,6-dimethoxyphenyl, or 2,4-dimethoxyphenyl; wherein R³² is further optionally substituted with a solubilizing group.

In certain embodiments, R³² is not unsubstituted thienyl; unsubstituted phenyl; 2-methylphenyl; 4-fluorophenyl; 4-methoxyphenyl; 4-methylphenyl; 3,4-dioxyethylenephenyl; 3-acetylamino-4-methylphenyl; 3-[(6-amino-1-oxohexyl)amino]-4-methylphenyl; 3-amino-4-methylphenyl; 3,5-dimethoxyphenyl; 3-halo-4-methoxyphenyl; 3-nitro-4-methylphenyl; or 4-propoxyphenyl.

In one aspect, the invention provides [symbol] -modulating compounds of Structural Formula (XXVI):

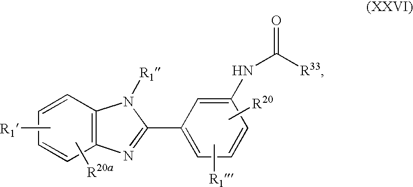

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl; and
R³³ is selected from an optionally substituted heteroaryl or an optionally substituted bicyclic aryl, with the provisos that:
when each of R₁′ and R₁′″ is hydrogen or methyl and each of R₁,″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl,

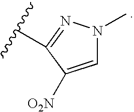

or

In a particular aspect, the invention provides [symbol] -modulating compounds of Structural Formula (XXVI):

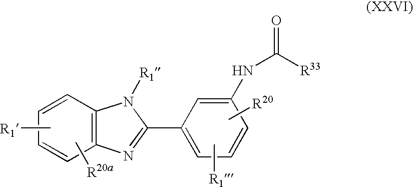

or a salt thereot, wheren:
each R²⁰ and R^{20a} is independently selected from H or a solubilizing group, wherein at least one of R²⁰ or R^{20a} is a solubilizing group;
each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] -modulating compounds of Structural Formula (XXVII):

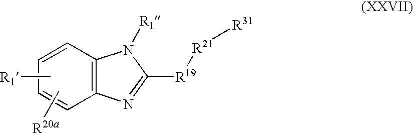

wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

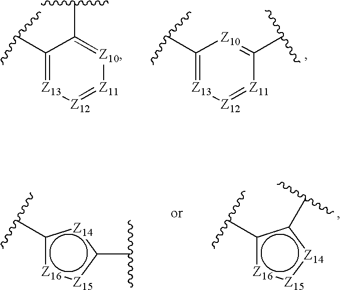

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁ₗ, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

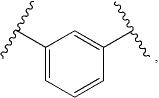

R³¹ is not unsubstituted pyridyl, 2,6-dimethoxyphenyl, 3,4,5-trimethoxyphenyl or unsubstituted furyl.

In a particular aspect, the invention provides [symbol] -modulating compounds of Structural Formula (XXVII):

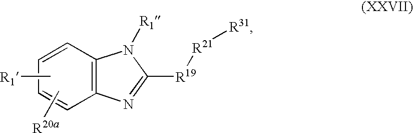

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

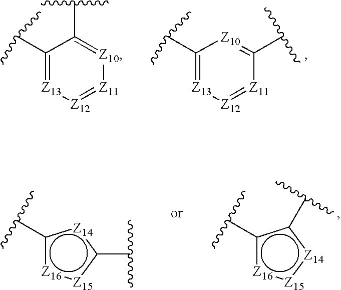

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—, and R¹⁹ is thiazolyl, R³¹ is not optionally substituted phenyl or optionally substituted pyridyl;
when R²¹ is —NH—C(O)—CH₂—, and R¹⁹ is pyrazolyl, R³¹ is not unsubstituted indolyl or unsubstituted phenyl;
when R²¹ is —NH—C(O)—CH₂—, and R¹⁹ is

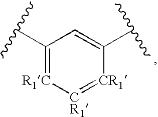

R³¹, is not 2-methylphenyl or 3,4-dimethoxyphenyl;
when R²¹ is —NH—C(O)—CH═CH—, and R¹⁹ is

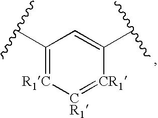

R³¹ is not 2-chlorophenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;
when R²¹ is —NH—C(O)—NH—, and R¹⁹ is

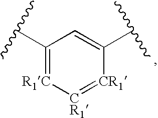

R³¹ is not unsubstituted benzimidazolyl;
when R²¹ is —NH—, and R¹⁹ is pyrazolyl, R³¹ is not unsubstituted pyridyl;
when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;
when R²¹ is —NH— and R¹⁹ is pyridyl, oxadiazolyl or thiadiazolyl, R³¹ is not unsubstituted phenyl, 3-methoxyphenyl or 4-methoxyphenyl;
when R²¹ is —NH—C(O)— and R¹⁹ is thiazolyl or pyrimidinyl, R³¹ is not unsubstituted phenyl;
when R²¹ is —NH—C(O)— and R¹⁹ is

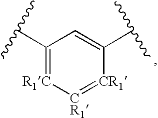

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

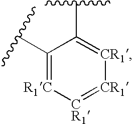

R³¹ is not 3,5-dinitrophenyl, 4-butoxyphenyl,

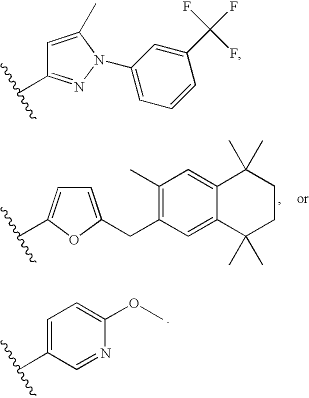

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In certain embodiments, when R²¹ is —NH—C(O)—, R¹⁹ is not

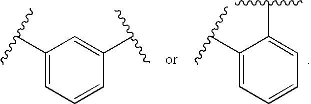

In certain embodiments, when R²¹ is —NH—C(O)—, R¹⁹ is not optionally substituted pyrazolyl, thiazolyl, thienyl, pyrrolyl or pyrimidinyl; when R²¹ is —NH—C(O)—CH₂— or —NH—C(O)—NH—, R¹⁹ is not pyrazolyl; and/or when R²¹ is —NH—, R¹⁹ is not optionally substituted pyridyl, thiazolyl, pyrazolyl, thiadiazolyl, or oxadiazolyl.

In a more particular aspect, the invention provides [symbol] -modulating compounds of Structural Formula (XXVII):

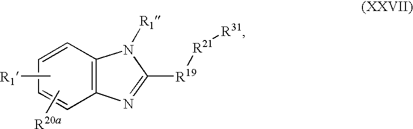

or a salt thereof, wherein:
each R²⁰ and R^{20a} is independently selected from H or a solubilizing group;

each R₁′ and R₁″ is independently selected from H or optionally substituted C₁-C₃ straight or branched alkyl;

R¹⁹ is selected from:

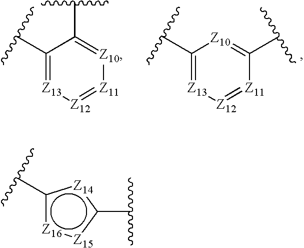

or

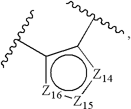

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
one to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁)—NR₁′—, —NR₁′—C(O)—CR₁′═CR₁′—, —NR₁′—S(O)—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—C(O)—CH₂—, and R¹⁹ is pyrazolyl, R³¹ is not unsubstituted indolyl or unsubstituted phenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;
when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;
when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and
when R²¹ is —NH—C(O)— and R¹⁹ is thiazolyl or pyrimidinyl, R³¹ is not unsubstituted phenyl.

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl.

Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In yet another aspect, the invention provides compounds of Structural Formula (XXVIII):

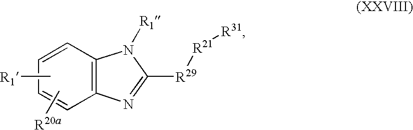

or a salt thereof, wherein:
each R²⁰ and R^{20a} is independently selected from H or a solubilizing group;
each R₁′ and R₁″ is independently selected from H or optionally substituted C₁-C₃ straight or branched alkyl;
R²⁹ is selected from:

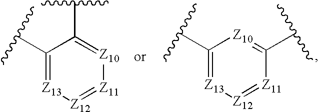

wherein:
each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl.

In certain embodiments, R³¹ is optionally substituted phenyl, such as 3-methoxyphenyl, 3,4-dimethoxyphenyl, 3,4,5-trimethoxyphenyl, or 4-dimethylaminophenyl.

In certain embodiments, R²¹ is —NH—C(O)—.

In preferred embodiments, R²¹ is —NH—C(O)— and R³¹ is an optionally substituted phenyl, such as 3-methoxyphenyl, 3,4-dimethoxyphenyl, 3,4,5-trimethoxyphenyl, or 4-dimethylaminophenyl.

In a further aspect, the invention provides novel [symbol] -modulating compounds of Formula (VI):

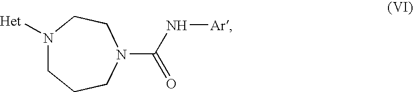

or a salt thereof, wherein:

Het is an optionally substituted heterocyclic aryl group; and

Ar′ is an optionally substituted carbocyclic or heterocyclic aryl group.

In certain embodiments, Het comprises one N heteroatom and 1 to 2 additional heteroatoms independently selected from N, O or S, such as oxazolopyridyl.

In certain embodiments, Ar′ is selected from optionally substituted phenyl, benzothiazolyl, or benzoxazolyl. When Ar′ is substituted phenyl, typically it is substituted with 1 to 3 substituents independently selected from halo, methyl, O-methyl, S-methyl or N(CH₃)₂, morpholino, or 3,4 dioxymethylene.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

In the compounds described above, bivalent groups disclosed as possible values for variables can have either orientation, provided that such orientation results in a stable molecule. Preferably, however, the left hand side of a bivalent group (e.g., —NR₁′—C(O)—) is attached to a bivalent arylene or heteroarylene group (e.g., R³¹) and the right hand side of a bivalent group is attached to a monovalent aryl group (e.g., R³¹).
[symbol] modulating compounds of the invention having hydroxyl substituents, unless otherwise indicated, also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

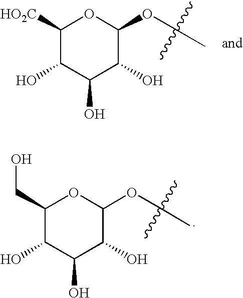

These groups are generally cleavable to —OH by hydrolysis or by metabolic (e.g., enzymatic) cleavage.

In certain embodiments, the compounds of the invention exclude one or more of the species disclosed in Tables 4-6. In certain such embodiments, the compounds of the invention exclude compound 7.
[symbol] modulating compounds of the invention advantageously [symbol] modulate the level and/or activity of a protein.

Separately or in addition to the above properties, certain [symbol] -modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A ring (e.g., 5- to 7-membered ring) or cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuryl, tetrahydrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}; —NRCOR^{a}, —NHCONH₂, —NHCONR^{a}H, —NHCON(R^{a}R^{b}), —NRCCONH₂, —NRCCONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR₀, —SO₂NR^{a}R^{b}, —CH═CHR^{a}, —CH═CR^{a}R^{b}, CR^{c}═CR^{a}R^{b}, CR═CHR^{a}, —CR^{c}═R^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

A hydrogen-bond donating group is a functional group having a partially positively-charged hydrogen atom (e.g., —OH, —NH₂, —SH) or a group (e.g., an ester) that metabolizes into a group capable of donating a hydrogen bond.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R^{a}—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C6-C14) aryl, phenyl, naphthyl, (C7-C20) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)C(O)—R^{b}, —(CH₂)—O—(CH₂)—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or R^{b} moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula: —(CH₂)—R¹⁰⁰—N(R¹⁰¹)(R¹⁰¹), wherein:

n is selected from 0, 1 or 2;
R¹⁰⁰ is selected from a bond, —C(O)—, or —O(CH₂)ₙ; and
each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;
c.

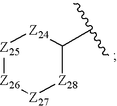

d.

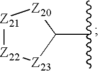

e.)

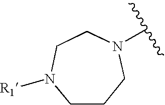

f. both R¹⁰¹ moieties are taken together with the nitrogen atom to which they are bound to form a ring of the structure

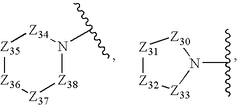

or

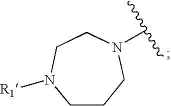

or
g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′; wherein:

each Z is independently selected from —O—, —S—, —NR₁′—, or —C(R⁵⁰)(R⁵⁰)—, wherein:
at least three of Z₂₀, Z₂₁, Z₂₂, and Z₂₃ are —C(R⁵⁰)(R₅₀)—;
at least three of Z₂₄, Z₂₅, Z₂₆, Z₂₇, and Z₂₈ are —C(R⁵⁰)(R⁵⁰)—;
at least four of Z₃₀, Z₃₁, Z₃₂, and Z₃₃ are —C(R⁵⁰)(R⁵⁰)—; and
at least four of Z₃₄, Z₃₅, Z₃₆, Z₃₇, and Z₃₈ are —C(R⁵⁰)(R⁵⁰)—;

each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;

each R⁵⁰ is independently selected from R₁′, halo, CN, OH, O—(C₁-C₄ straight or branched alkyl), N(R₁′)(R₁′), ═CR₁′, SR₁′, ═NR₁′, ═NOR₁′, or ═O;

any two suitable non-cyclic R⁵⁰ are optionally bound to one another directly or via a C₁ to C₂ alkylene, alkenylene or alkanediylidene bridge to produce a bicyclic fused or spiro ring; and any

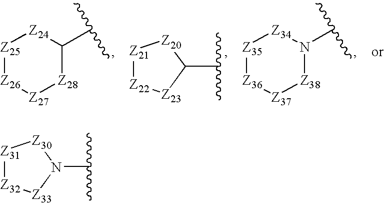

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another preferred embodiment, the solubilizing group is a moiety of the formula: —(CH₂)ₙ—O—R¹⁰¹, wherein n and R¹⁰¹ are as defined above.

In another preferred embodiment, the solubilizing group is a moiety of the formula: —(CH₂)ₙ—C(O)—R₁′, wherein n and R₁′ are as defined above.

In a more preferred embodiment, a solubilizing group is selected from —(CH₂)ₙ—R¹⁰², wherein n is 0, 1 or 2; and R¹⁰² is selected from

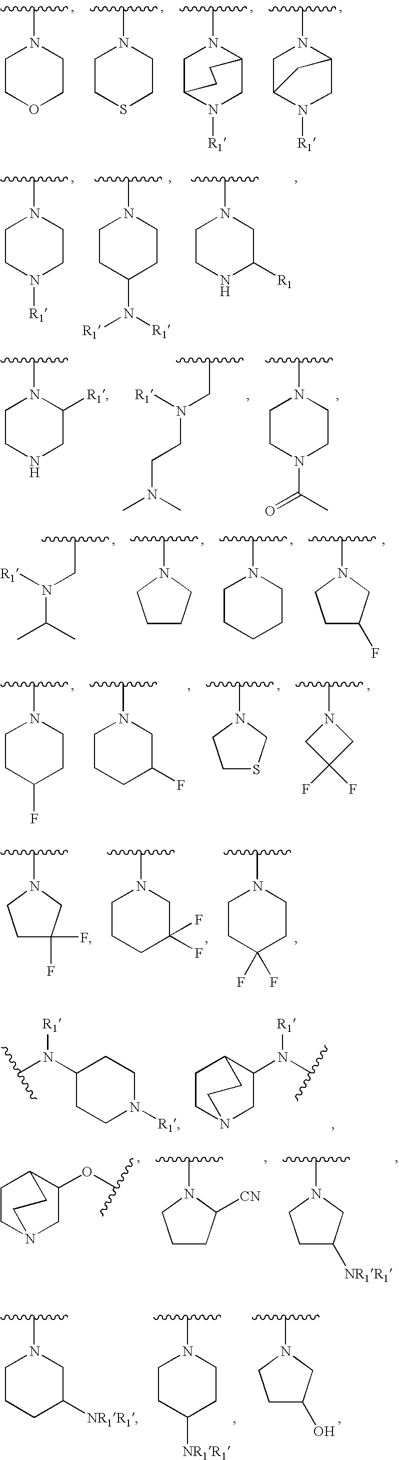

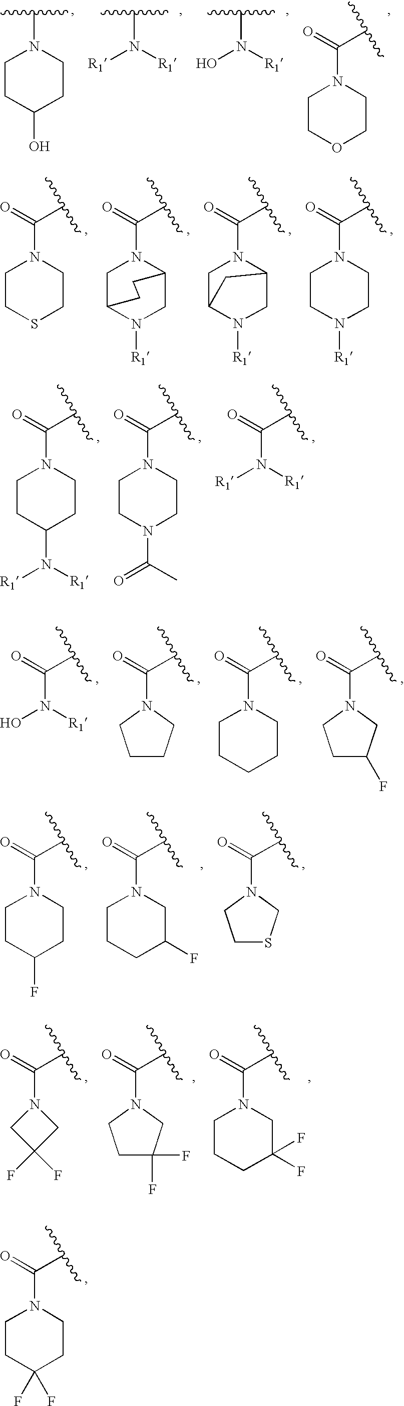

TABLE 4

| | | | ED₅₀ | ED₅₀ | FOLD |
|---|---|---|---|---|---|
| COMPOUND | | | FP | MS | ACT. |
| No | [M + H]+ | STRUCTURE | ASSAY | ASSAY | MS |
| 1 | | 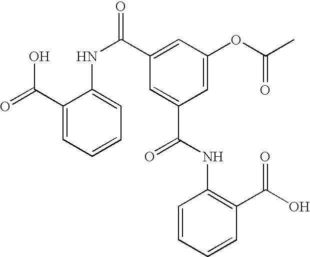 | A | NT | |
| 2 | | 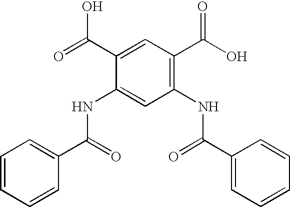 | D | NT | |
| 3 | | 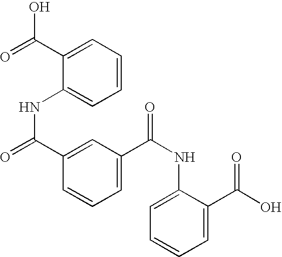 | B | NT | |
| 4 | | 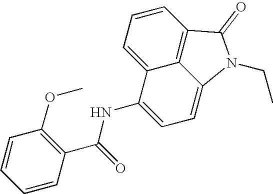 | N/A | NT | |
| 5 | | 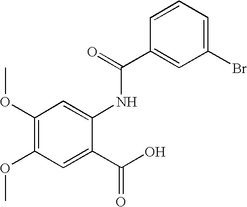 | B | NT | |
| 6 | | 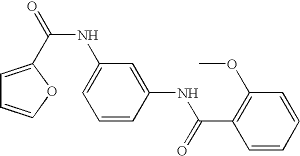 | D | NT | |
| 7 | 346 | 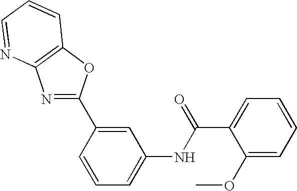 | A | NT | |
| 8 | | 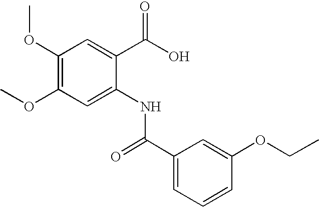 | B | NT | |
| 9 | | 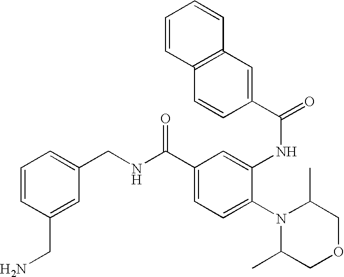 | C | NT | |
| 10 | | 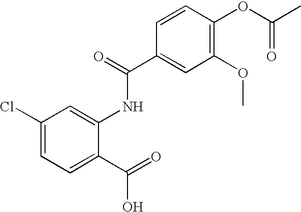 | D | NT | |
| 19 | 409.6 | 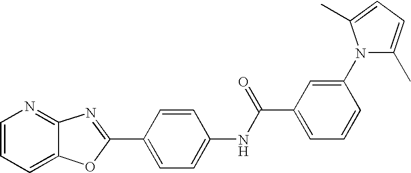 | D | D | C |
| 20 | 401.3 | 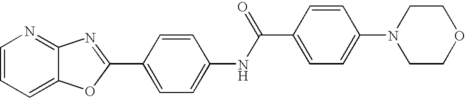 | D | D | C |
| 21 | 399.1 | 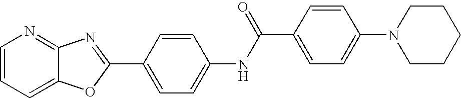 | D | D | C |
| 22 | 414.0 | 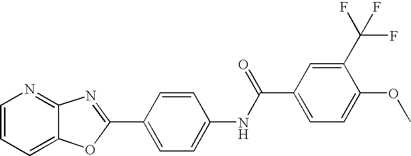 | D | D | C |
| 24 | 359.4 | 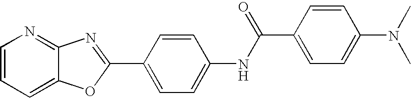 | D | D | C |
| 27 | 376.1 | 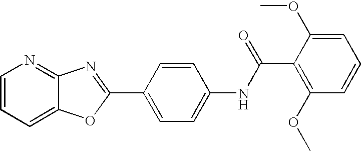 | D | D | C |
| 29 | 385.1 | 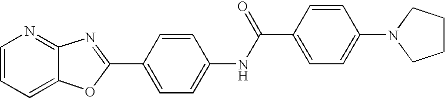 | D | D | C |
| 31 | 360.1 | 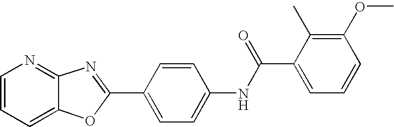 | D | D | C |
| 32 | 400.0 | 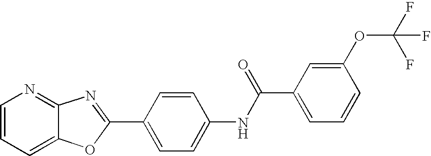 | D | D | C |
| 33 | 376.1 | 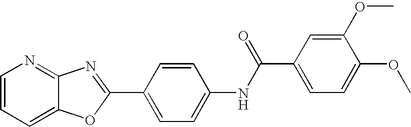 | D | D | C |
| 34 | 406.3 | 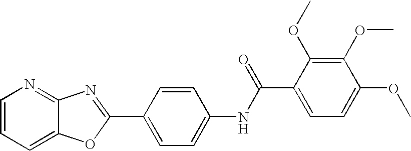 | D | D | C |
| 35 | 346.5 | 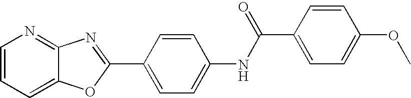 | D | D | C |
| 36 | 376.7 | 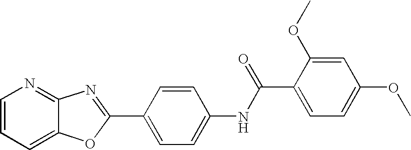 | D | D | C |
| 37 | 316.4 | 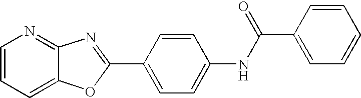 | D | D | C |
| 38 | 401.0 | 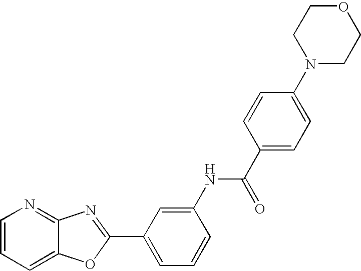 | D | D | C |
| 39 | 399.1 | 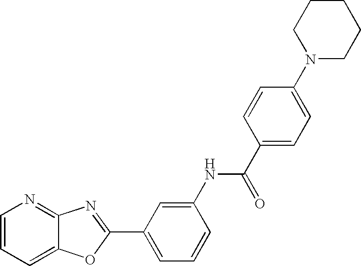 | D | D | C |
| 40 | 414.2 | 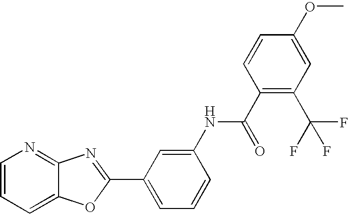 | D | D | C |
| 41 | 414.2 | 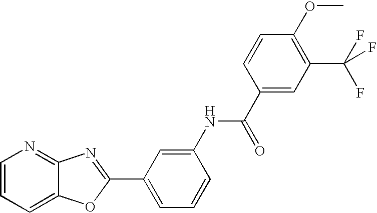 | D | D | C |
| 42 | 359.1 | 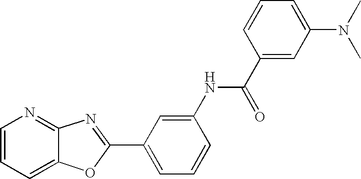 | A | A | B |
| 43 | 359.1 | 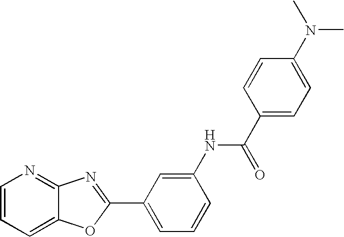 | A | A | B |
| 45 | 341.0 | 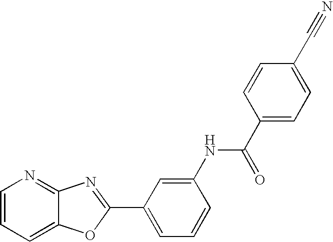 | D | D | C |
| 46 | 376.1 | 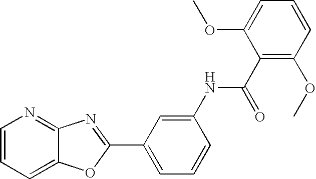 | D | D | C |
| 48 | 406.3 | 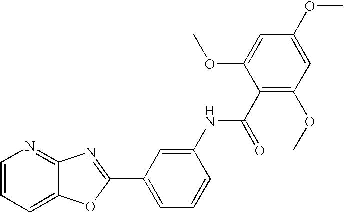 | D | D | C |
| 49 | 360.1 | 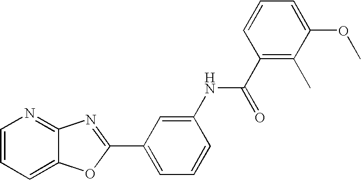 | D | A | B |
| 50 | 400.0 | 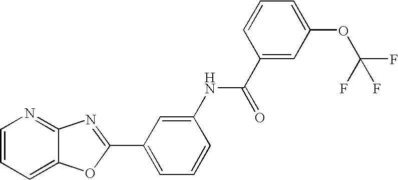 | NT | D | |
| 51 | 360.1 | 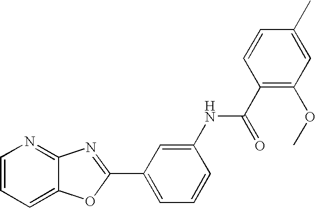 | A | A | B |
| 52 | 376.1 | 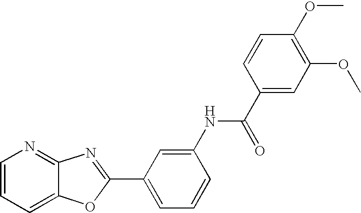 | A | A | B |
| 53 | 406.1 | 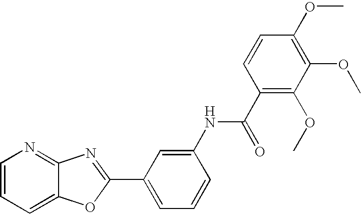 | D | D | C |
| 54 | 346.4 | 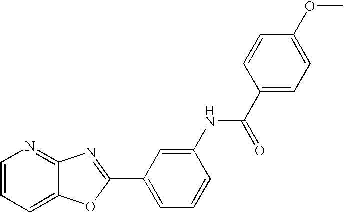 | NT | D | |
| 55 | 376.1 | 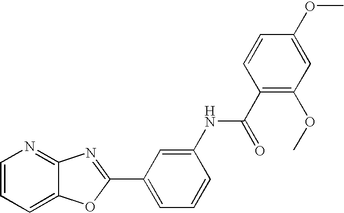 | A | A | B |
| 56 | 316.0 | 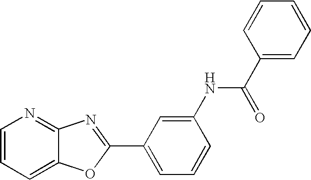 | D | A | B |
| 57 | 407.1 | 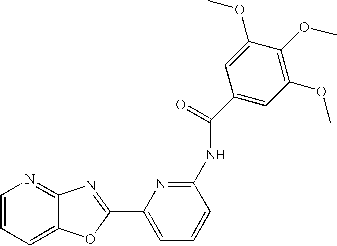 | | A | |
| 58 | 377.1 | 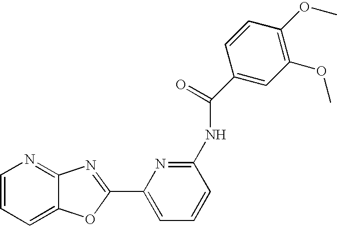 | | NA | |
| 59 | 318.1 | 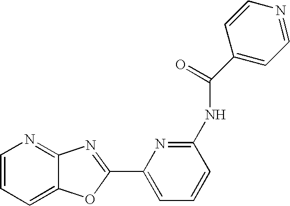 | | NA | |
| 60 | 413.1 | 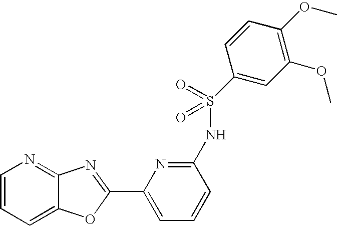 | | A | |
| 61 | 413.1 | 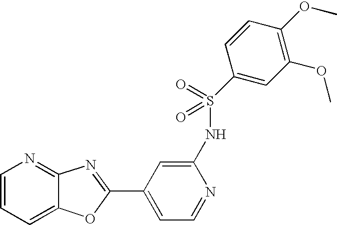 | | NA | |
| 62 | 349.0 | 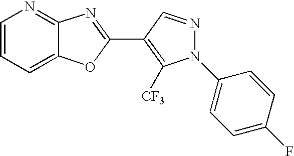 | | NA | |
| 63 | 377.1 | 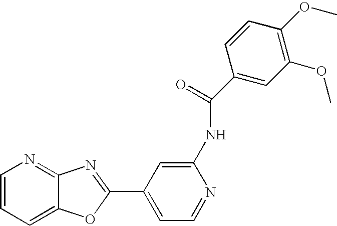 | | A | |
| 64 | 360.1 | 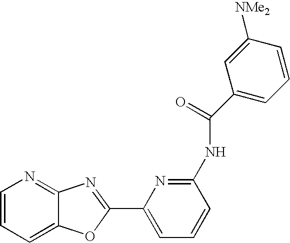 | | A | |
| 65 | 383.0 | 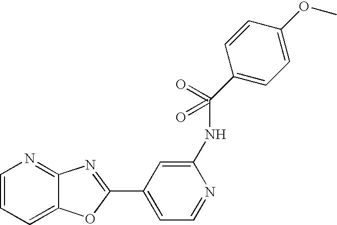 | | NA | |
| 66 | 407 | 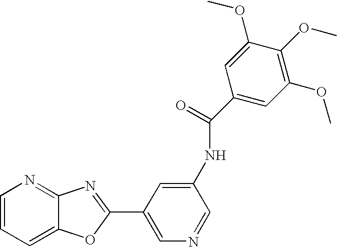 | | A | |
| 67 | 377 | 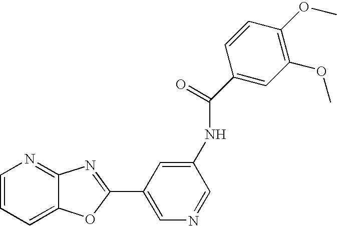 | | A | |
| 68 | 360 | 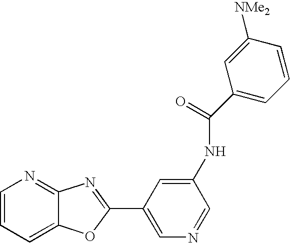 | | A | |
| 69 | 377.1 | 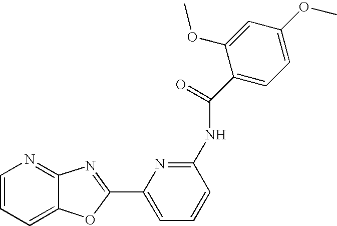 | | A | B |
| 70 | 360.1 | 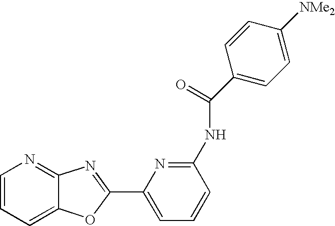 | | A | B |
| 71 | 376.1 | 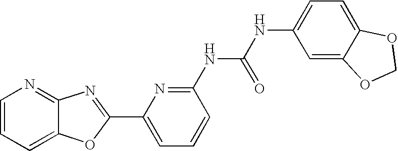 | | A | B |
| 72 | 422.1 | 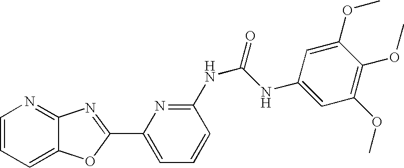 | | NT | |
| 73 | 377 | 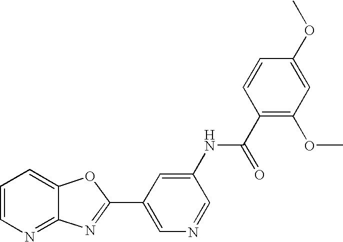 | | NT | |
| 74 | 412 | 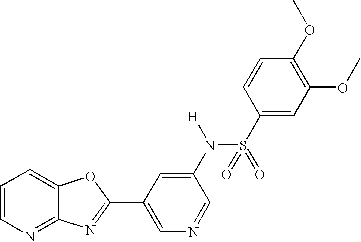 | | D | C |
| 75 | 407.1 | 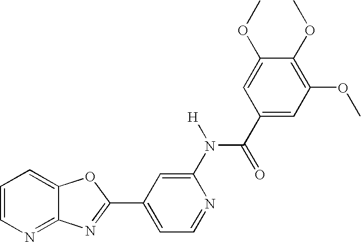 | | A | B |
| 76 | 360.1 | 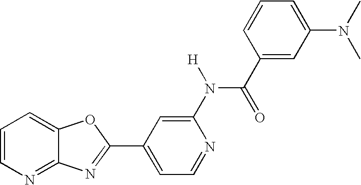 | | A | B |
| 77 | 376.1 | 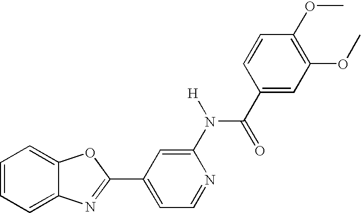 | | A | B |
| 78 | 445.1 | 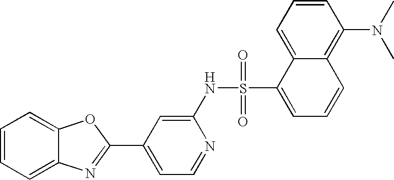 | | | |
| 79 | 338 | 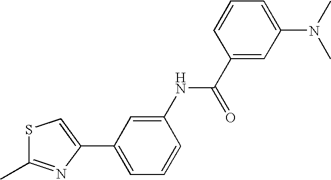 | | D | C |
| 80 | 355 | 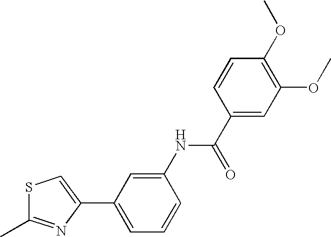 | | A | B |
| 81 | 354.5 | 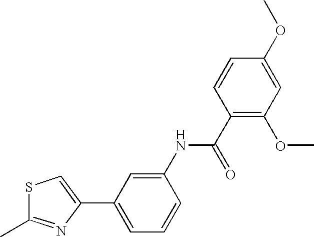 | | A | B |
| 82 | 402 | 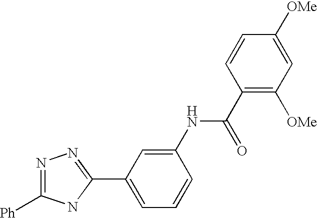 | | C | |
| 83 | 355 | 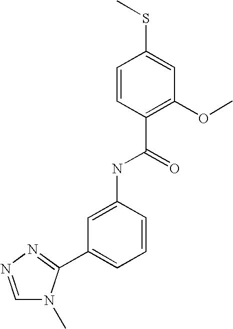 | | A | B |
| 84 | 417 | 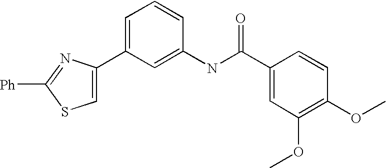 | | A | B |
| 85 | 335.1 | 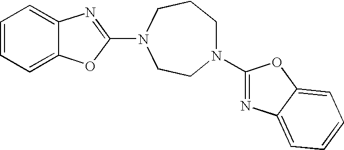 | | D | C |
| 86 | 375.1 | 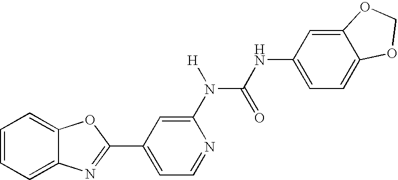 | | D | C |
| 87 | 375.1 | 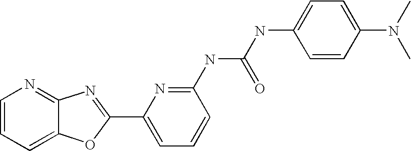 | | D | C |
| 88 | 382.1 | 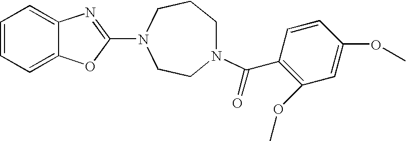 | | D | C |
| 89 | 365.1 | 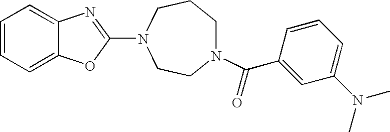 | | D | C |
| 90 | 381.1 | 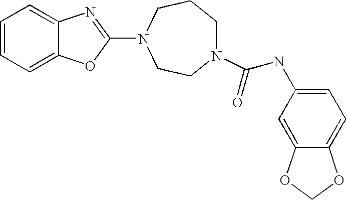 | | | |
| 91 | 412.1 | 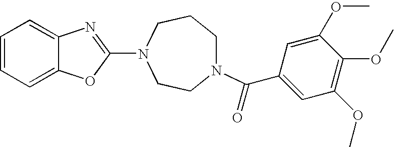 | | D | C |
| 92 | 308.1 | 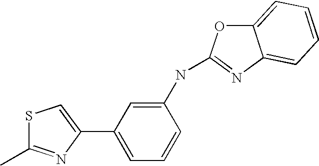 | | A | B |
| 93 | 329.1 | 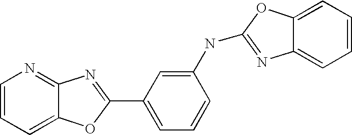 | | A | B |
| 94 | 434.1 | 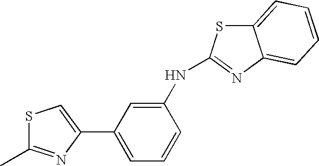 | | A | B |
| 95 | 345.1 | 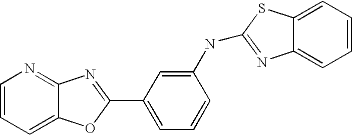 | | A′ | B |
| 96 | 376.1 | 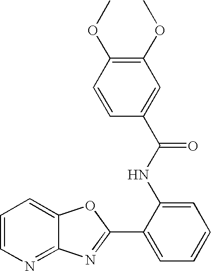 | | A′ | B |
| 97 | 359.1 | 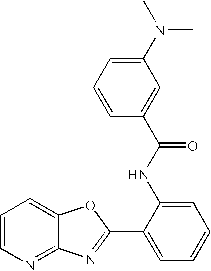 | | A′ | B |
| 98 | 408.1 | 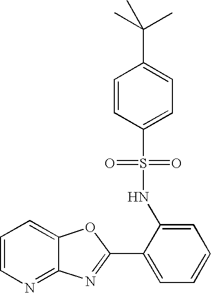 | | D | C |
| 99 | 391 | 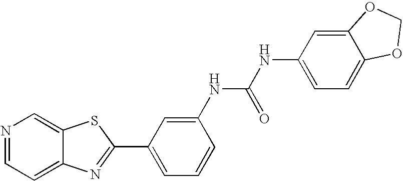 | | A′ | A |
| 100 | 376 | 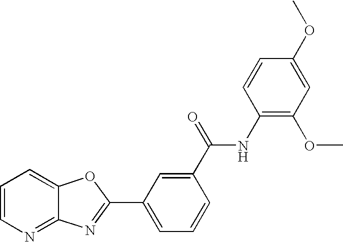 | | A | B |
| 101 | 376 | 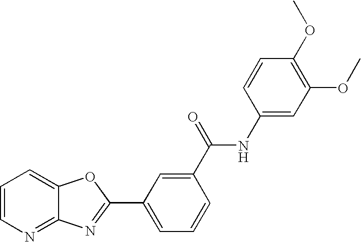 | | A | A |
| 102 | 406 | 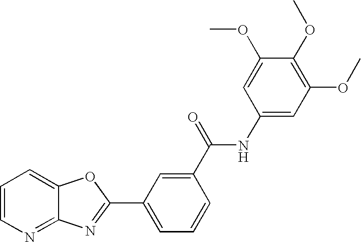 | | A | A |
| 103 | 374 | 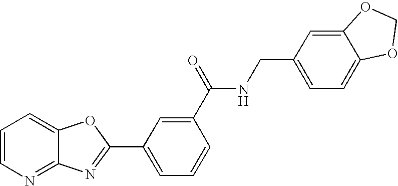 | | D | C |
| 104 | 346.1 | 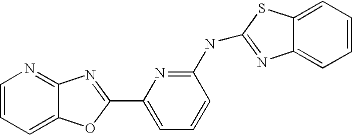 | | A′ | B |
| 105 | 330.1 | 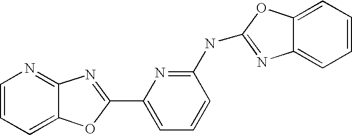 | | A′ | B |
| 106 | 406.1 | 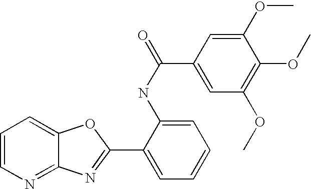 | | A′ | B |
| 107 | 488 | 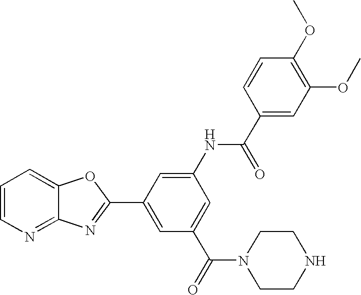 | | A | B |
| 108 | 324 | 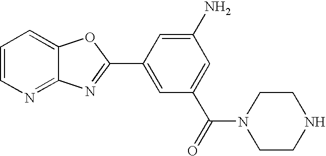 | | NA | |
| 109 | 401 | 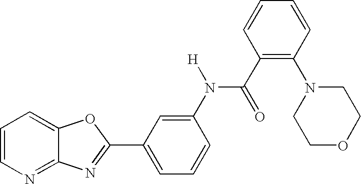 | | A | B |
| 110 | 381 | 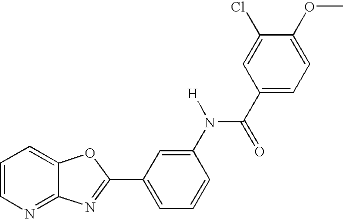 | | C | |
| 111 | 359 | 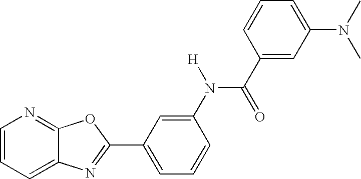 | | A | B |
| 112 | 376 | 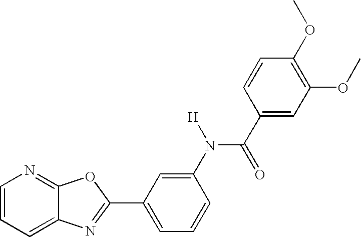 | | A | B |
| 113 | 375 | 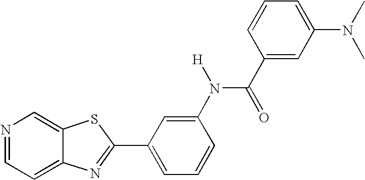 | | A | B |
| 114 | 392 | 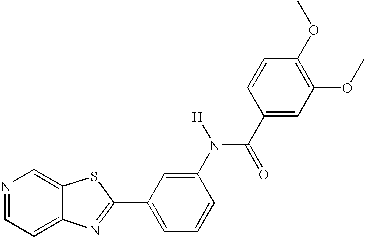 | | A′ | A |
| 115 | 422 | 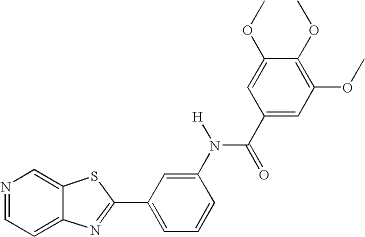 | | A | A |
| 116 | 386 | 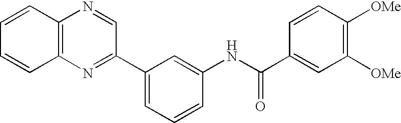 | | C | |
| 117 | 388 | 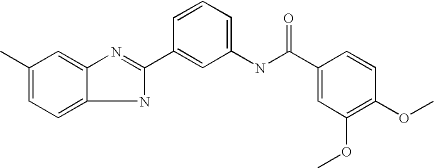 | | A | A |
| 118 | 410 | 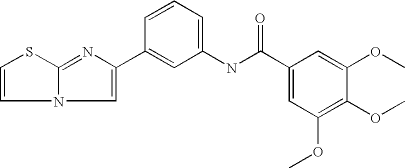 | | A | A |
| 119 | 375 | 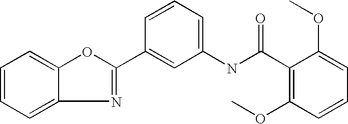 | | A | B |
| 120 | 391 | 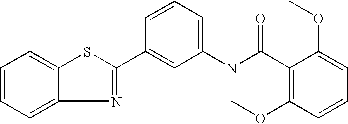 | | NT | |
| 121 | 414 | 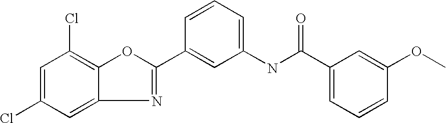 | | D | C |
| 122 | 417 | 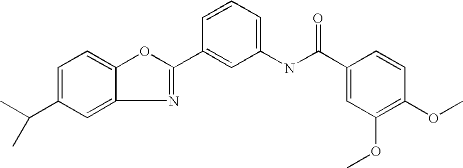 | | C | |
| 123 | 474 | 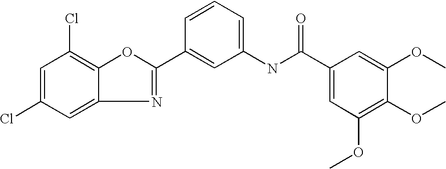 | | NT | |
| 124 | 391 | 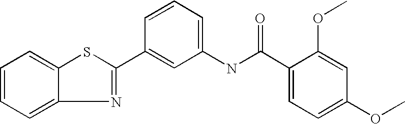 | | A | B |
| 125 | 433 | 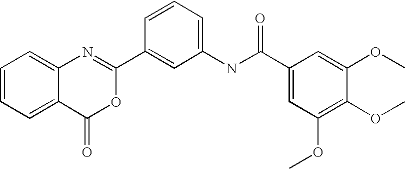 | | B | B |
| 126 | 374 | 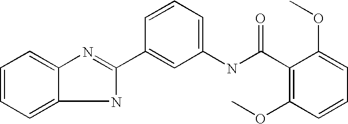 | | A | B |
| 127 | 355 | 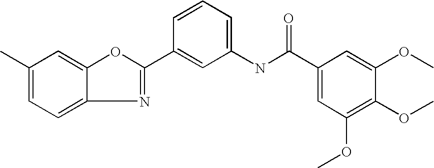 | | A | A |
| 128 | 388 | 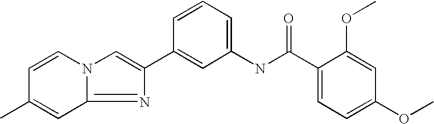 | | A | B |
| 129 | 418 | 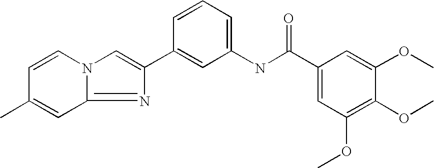 | | A′ | A |
| 130 | 358 | 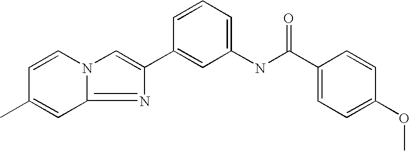 | | A | B |
| 131 | 418 | 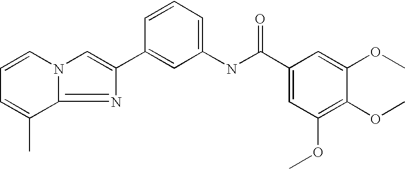 | | A | A |
| 132 | 350 | 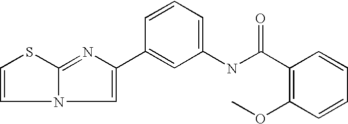 | | A | A |
| 133 | 480 | 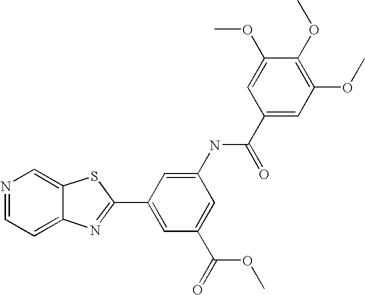 | | A′ | B |
| 134 | 466 | 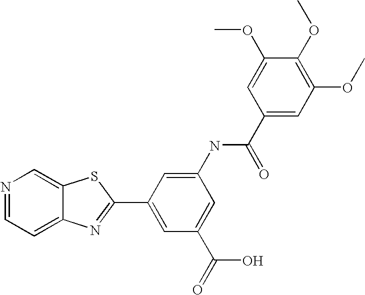 | | A′ | B |
| 135 | 452 | 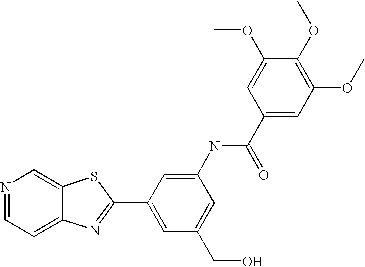 | | A′ | B |
| 136 | 434 | 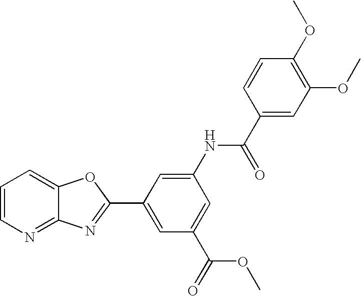 | | D | C |
| 137 | 420 | 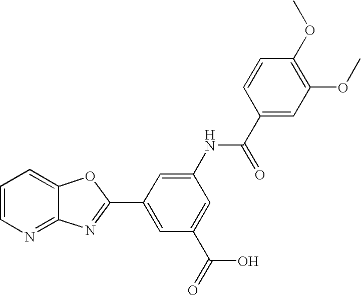 | | D | |
| 138 | 471 | 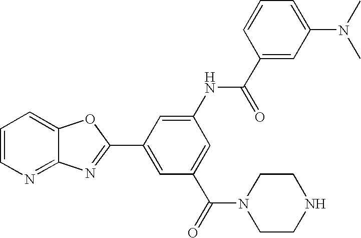 | | A | B |
| 139 | 488 | 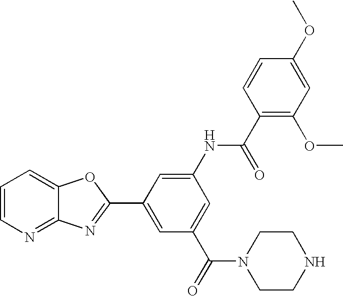 | | A | B |
| 141 | 374.1 | 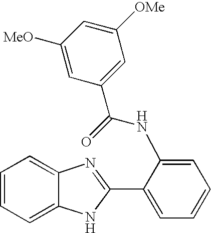 | | A′ | B |
| 142 | 374.1 | 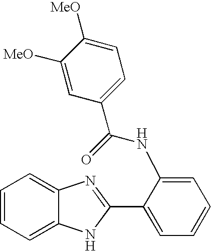 | | A′ | A |
| 143 | 410.1 | 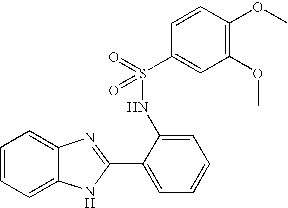 | | D | |
| 144 | 427.1 | 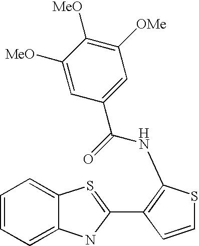 | | D | C |
| 145 | 397.1 | 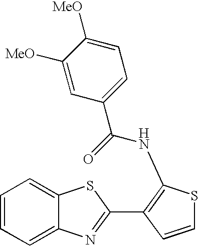 | | A′ | B |
| 146 | 397.1 | 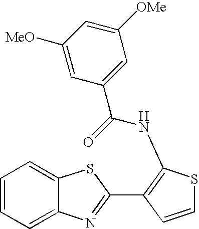 | | A′ | B |
| 147 | 392.1 | 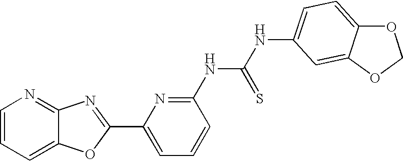 | | D | |
| 148 | 405.2 | 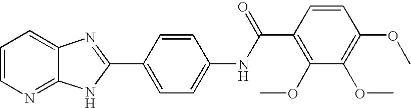 | | D | |
| 149 | 359.0 | 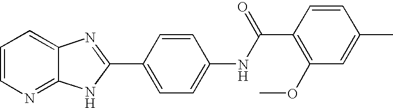 | | A′ | B |
| 150 | 375.0 | 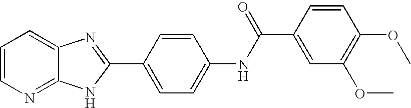 | | A′ | B |
| 151 | 375.0 | 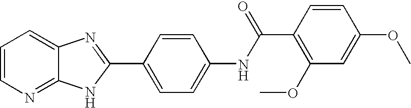 | | D | C |
| 152 | 392.1 | 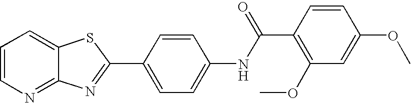 | | D | C |
| 153 | 490 | 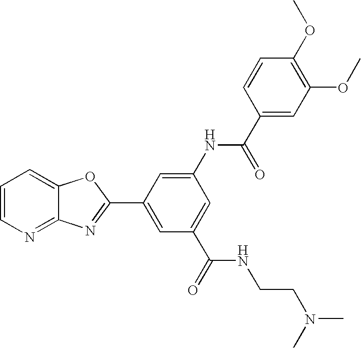 | | A | B |
| 154 | 473 | 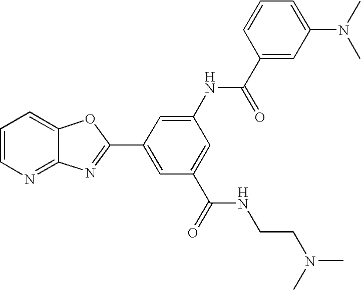 | | C | A |
| 155 | 490 | 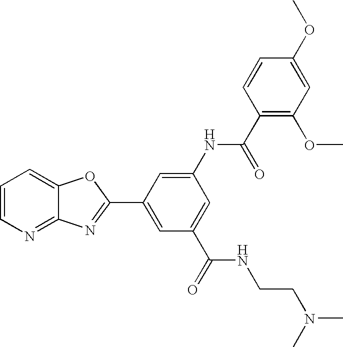 | | A | B |
| 156 | 433 | 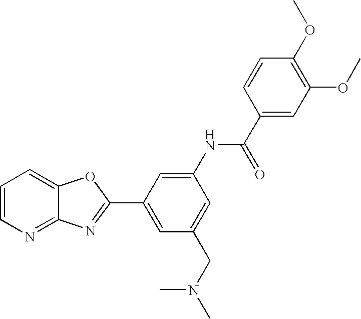 | | A | B |
| 157 | 416 | 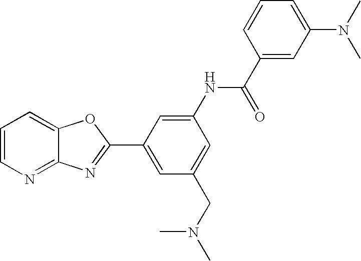 | | D | B |
| 158 | 433 | 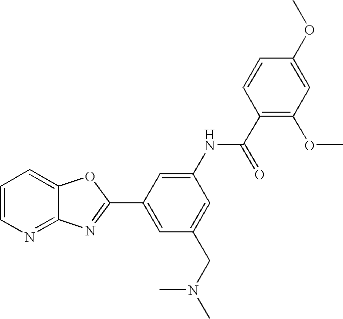 | | A | B |
| 159 | 474 | 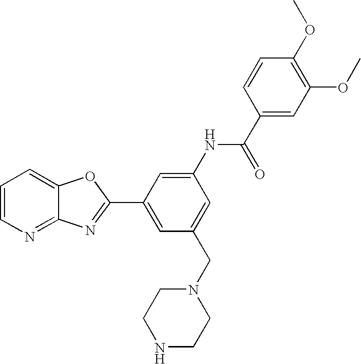 | | A | B |
| 160 | 457 | 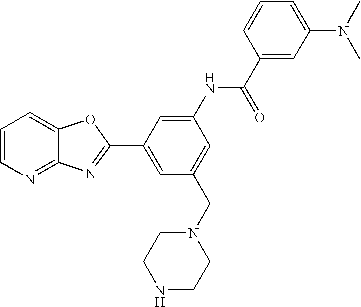 | | B | A |
| 161 | 474 | 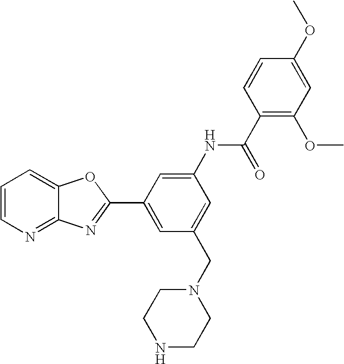 | | A | A |
| 162 | 392.1 | 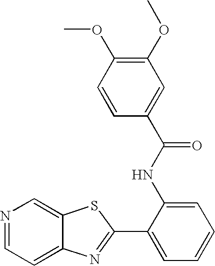 | | A′ | B |
| 163 | 422.1 | 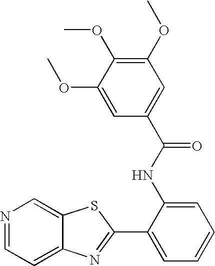 | | A′ | B |
| 164 | 488 | 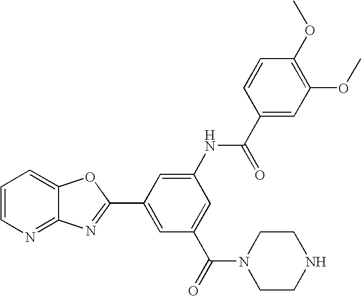 | | A | B |
| 165 | 416 | 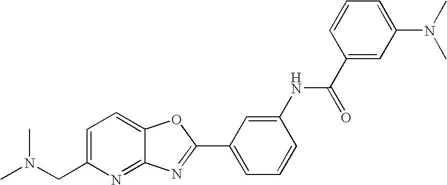 | | D | A |
| 166 | 457 | 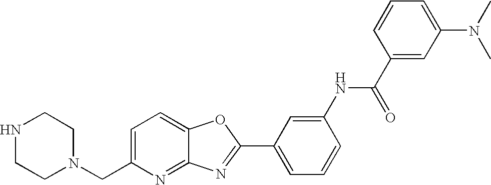 | | A | B |
| 167 | 373 | 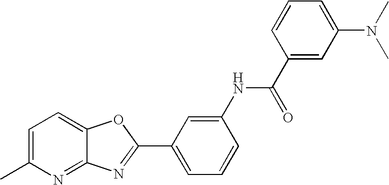 | | A | B |
| 168 | 388.1 | 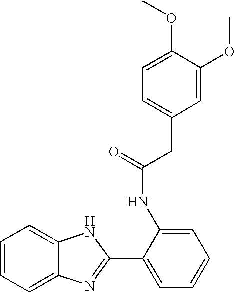 | | NA | C |
| 169 | 343.1 | 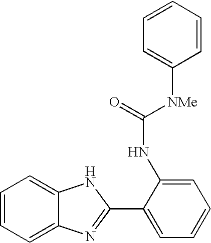 | | A′ | B |
| 174 | 479 | 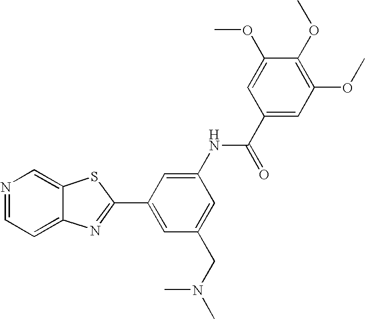 | | B | A |
| 175 | 323.1 | 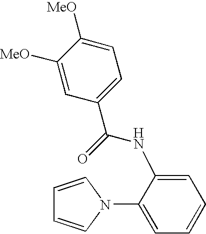 | | B | B |
| 176 | 354.1 | 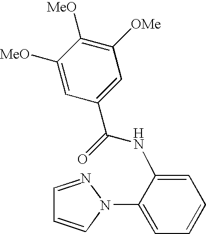 | | B | B |
| 177 | 324.1 | 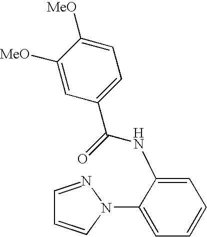 | | D | B |
| 178 | 437 | 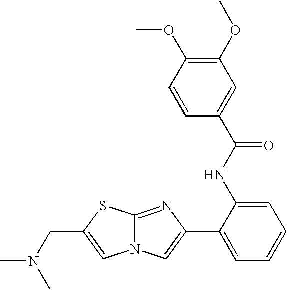 | | A | A |
| 179 | 467 | 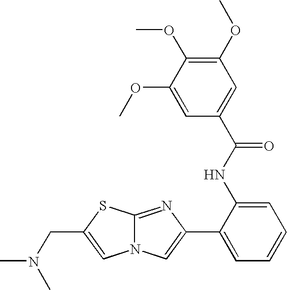 | | A′ | A |
| 180 | 358.0 | 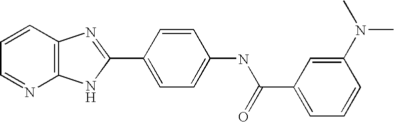 | | A | B |
| 181 | 405.0 | 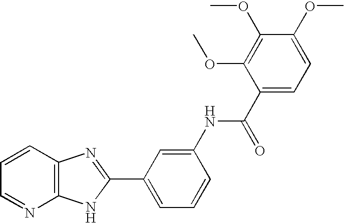 | | A′ | A |
| 182 | 359.0 | 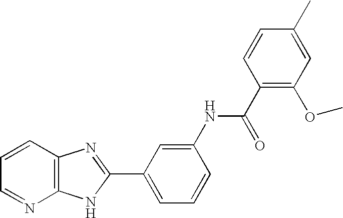 | | A′ | A |
| 183 | 358.0 | 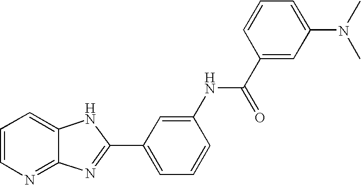 | | A | A |
| 184 | 375.0 | 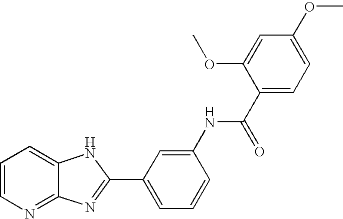 | | A′ | B |
| 185 | 374.9 | 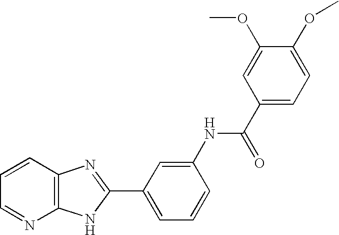 | | A′ | A |
| 186 | 405.3 | 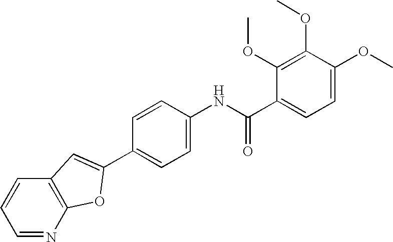 | | NA | C |
| 187 | 358.9 | 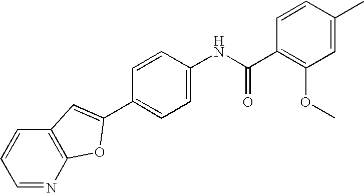 | | A | B |
| 188 | 358.9 | 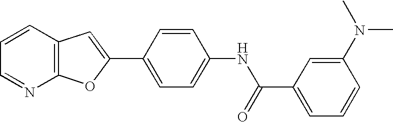 | | NA | C |
| 189 | 375.2 | 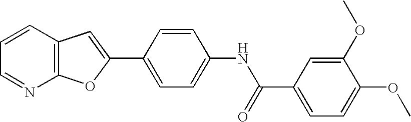 | | NA | C |
| 190 | 375.3 | 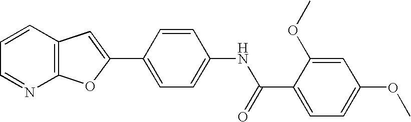 | | A′ | B |
| 191 | 375.0 | 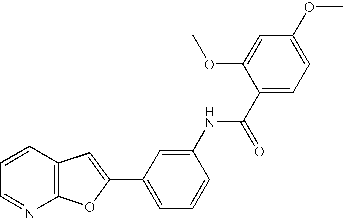 | | A′ | B |
| 192 | 375.0 | 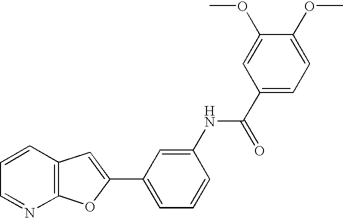 | | A | B |
| 193 | 358.0 | 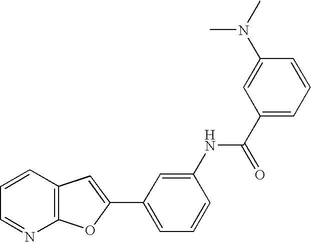 | | NA | C |
| 194 | 422.3 | 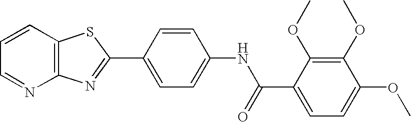 | | NA | C |
| 195 | 375.9 | 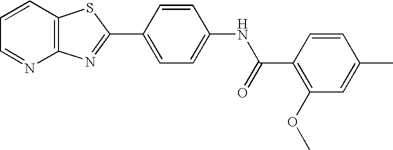 | | NA | C |
| 196 | 374.9 | 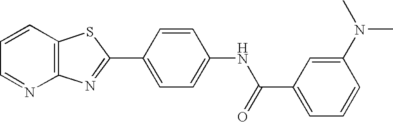 | | A | B |
| 197 | 391.1 | 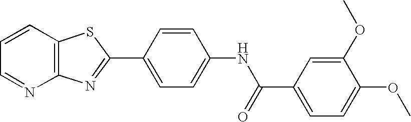 | | A′ | B |
| 198 | 422.4 | 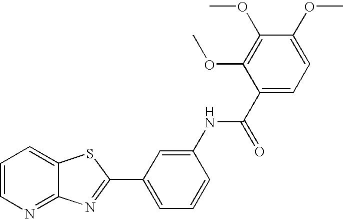 | | A | B |
| 199 | 375.9 | 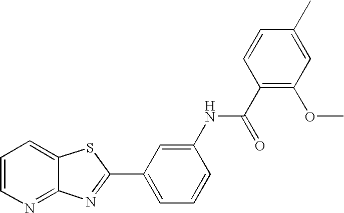 | | A′ | B |
| 200 | 375.4 | 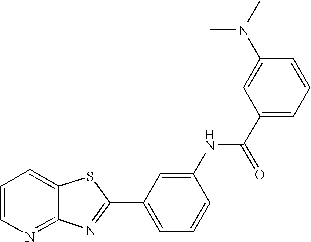 | | A′ | B |
| 201 | 391.9 | 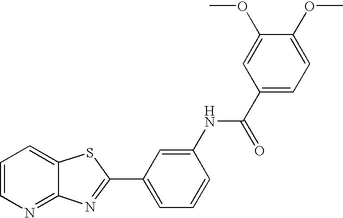 | | A | B |
| 202 | 392.4 | 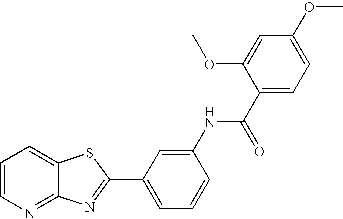 | | A′ | B |
| 203 | 380 | 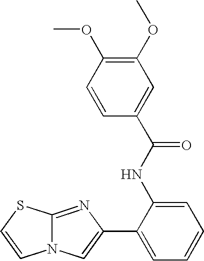 | | A′ | B |
| 204 | 410 | 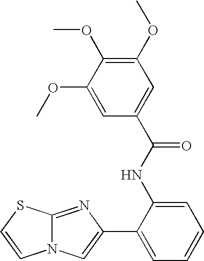 | | A′ | A |
| 205 | 437 | 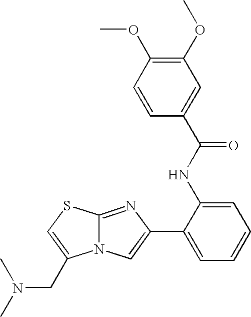 | | A | A |
| 206 | 467 | 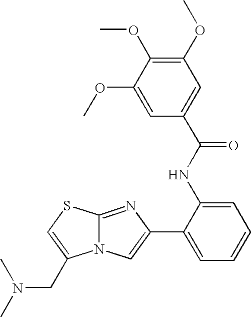 | | A | A |
| 207 | 478 | 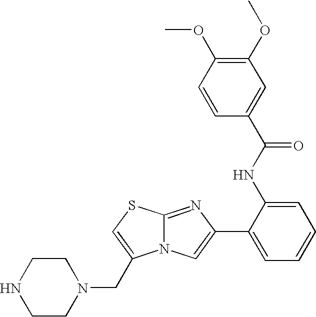 | | A | A |
| 208 | 508 | 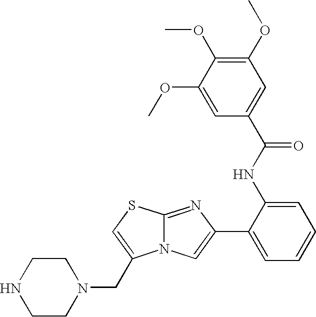 | | A | A |
| 209 | 479 | 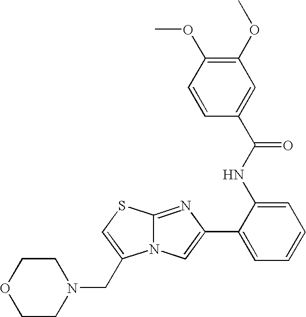 | | A | A |
| 210 | 509 | 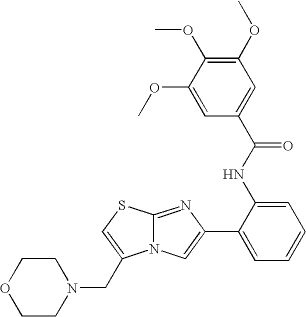 | | A | B |
| 211 | | 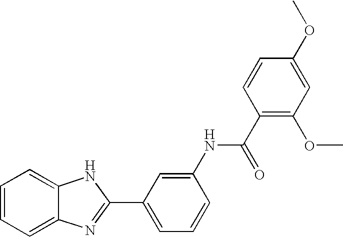 | | A′ | B |
| 212 | 362 | 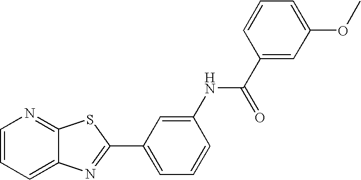 | | A | B |
| [symbol] | | | | | |
| 218 | 405 | 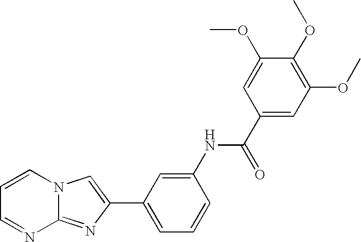 | | A′ | A |
| 219 | 419 | 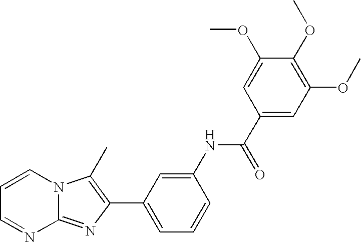 | | A | A |
| 220 | 423 | 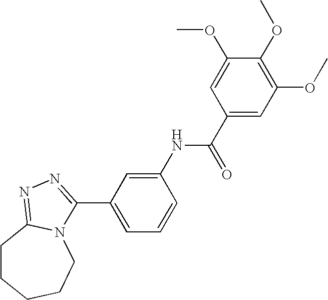 | | NA | C |
| 221 | 321.4 | 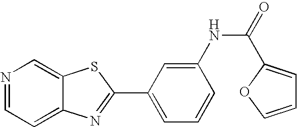 | | A | B |
| 222 | 366.8 | 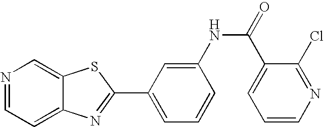 | | A | B |
| 223 | 337.4 | 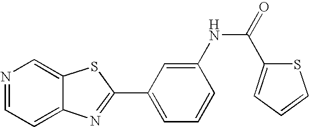 | | A′ | B |
| 225 | 332.4 | 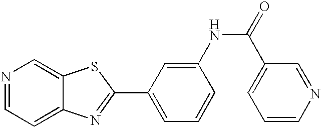 | | NA | C |
| 226 | 412.5 | 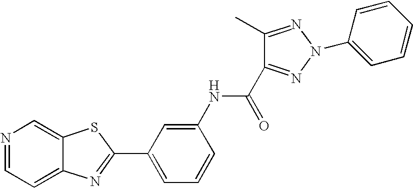 | | | |
| 227 | 333.4 | 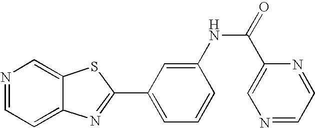 | | NA | C |
| 228 | 391.5 | 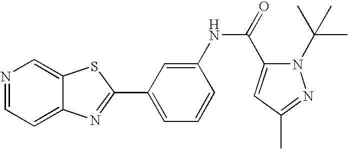 | | A | A |
| 229 | 418.5 | 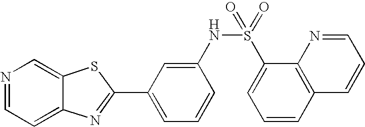 | | NA | C |
| 230 | 459.5 | 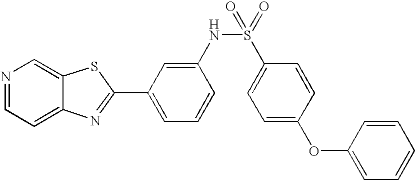 | | NA | C |
| 231 | 425.5 | 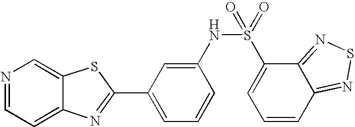 | | NA | C |
| 232 | 423.5 | 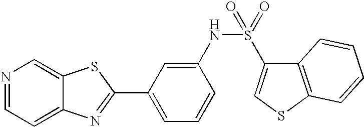 | | NA | C |
| 234 | 449.5 | 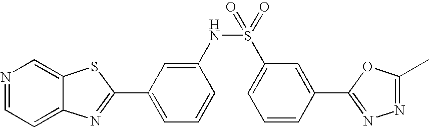 | | NA | C |
| 235 | 436.3 | 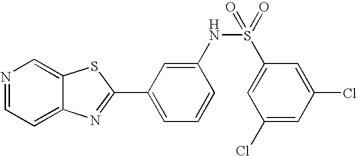 | | NA | C |
| 236 | 423.5 | 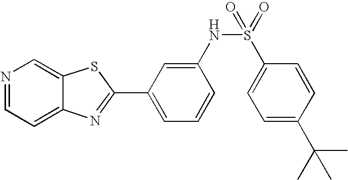 | | NA | C |
| 237 | 450.5 | 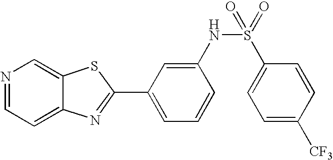 | | NA | C |
| 238 | 480.5 | 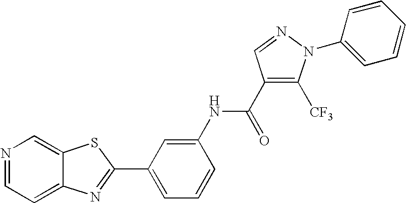 | | A′ | B |
| 239 | 466.5 | 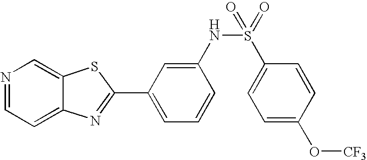 | | A | B |
| 240 | 392.4 | 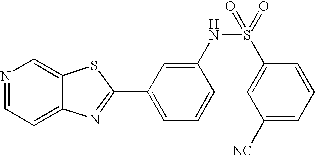 | | NA | C |
| 241 | 397.2 | 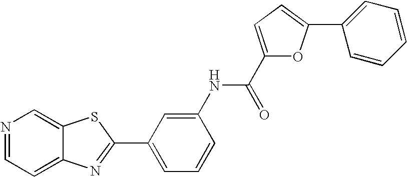 | | A′ | B |
| 244 | 332.4 | 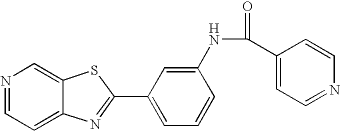 | | NA | C |
| 245 | 423.5 | 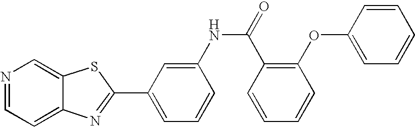 | | A′ | B |
| 246 | 391.5 | 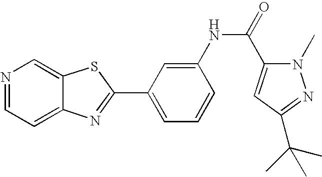 | | A′ | B |
| 247 | 413.5 | 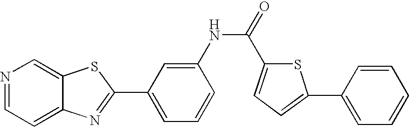 | | NA | C |
| 248 | 467.4 | 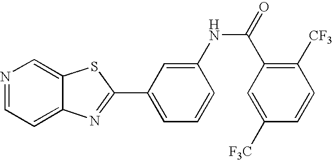 | | A | B |
| 249 | 395.9 | 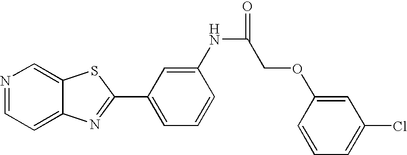 | | NA | C |
| 250 | 385.5 | 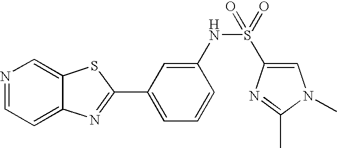 | | NA | C |
| 251 | 425.5 | 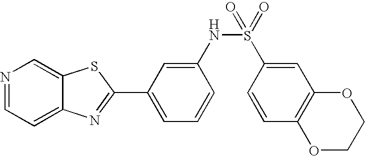 | | NA | C |
| 252 | 453.5 | 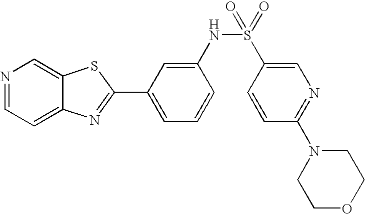 | | NA | C |
| 253 | 373.1 | 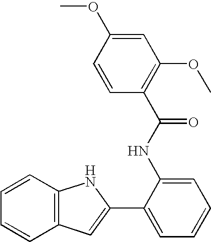 | | NA | C |
| 254 | 373 | 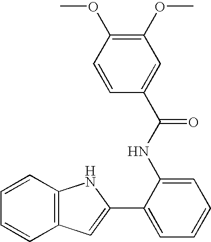 | | A′ | B |
| 255 | 403 | 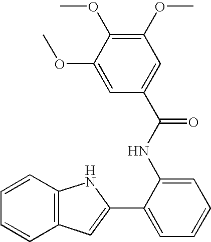 | | A′ | B |
| 256 | 356 | 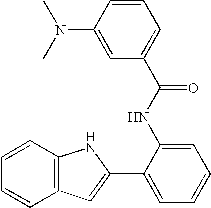 | | NA | C |
| 257 | 413.1 | 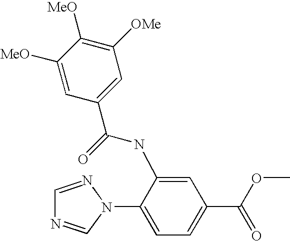 | | NA | C |
| 258 | 383.1 | 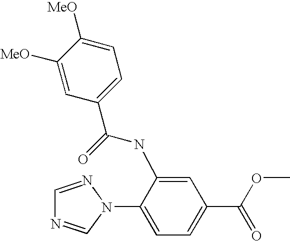 | | A | B |
| 259 | 405.1 | 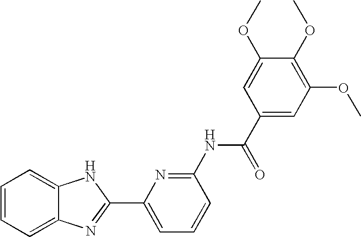 | | A′ | A |
| 260 | 375.1 | 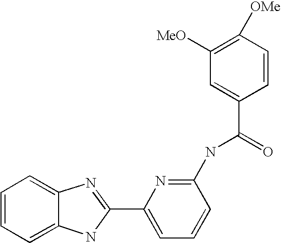 | | A′ | A |
| 261 | 375.1 | 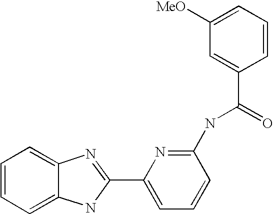 | | A′ | A |
| 262 | 374.1 | 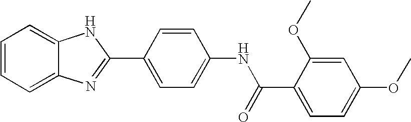 | | A′ | B |
| 263 | 404 | 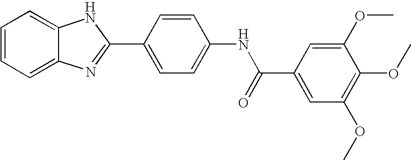 | | A | B |
| 264 | 357 | 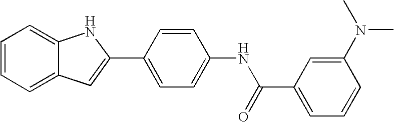 | | A′ | B |
| 265 | 374.1 | 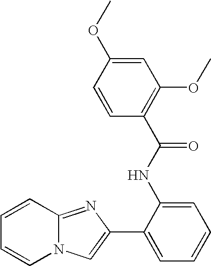 | | A | B |
| 266 | 374 | 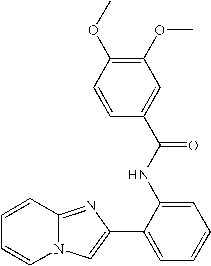 | | A′ | A |
| 267 | 404 | 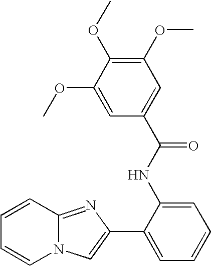 | | A′ | B |
| 268 | 357 | 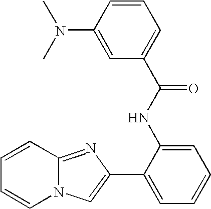 | | A′ | B |
| 270 | 478 | 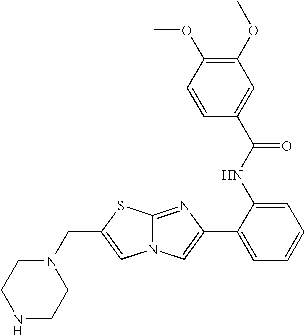 | | A | A |
| 271 | 508 | 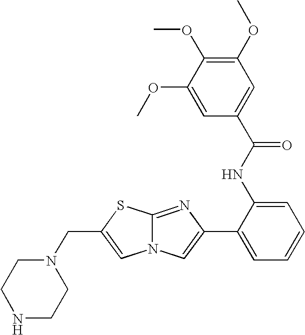 | | A′ | A |
| 272 | 392 | 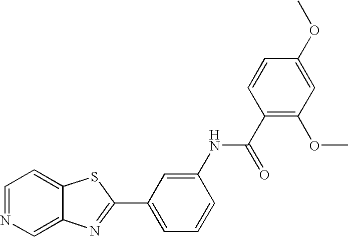 | | A′ | B |
| 273 | 422 | 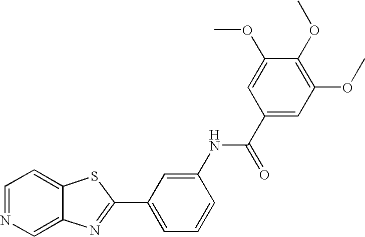 | | A | A |
| 276 | 375.1 | 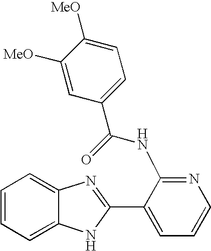 | | A′ | B |
| 280 | 381.5 | 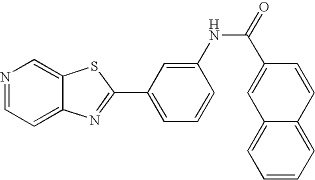 | | | |
| 282 | 386.5 | 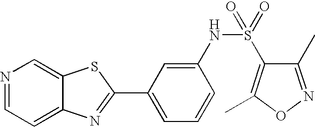 | | NA | C |
| 283 | 451.6 | 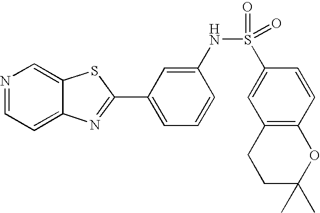 | | NA | C |
| 284 | 439.5 | 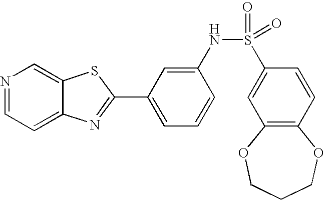 | | NA | C |
| 285 | 440.5 | 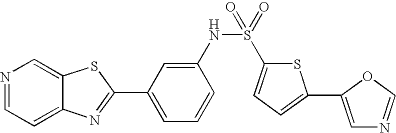 | | NA | C |
| 286 | 390.1 | 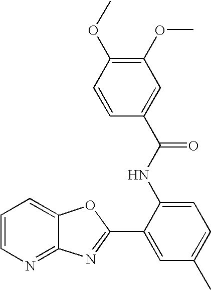 | | NA | C |
| 287 | 457.6 | 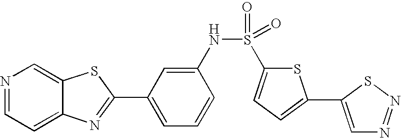 | | | |
| 288 | 424.5 | 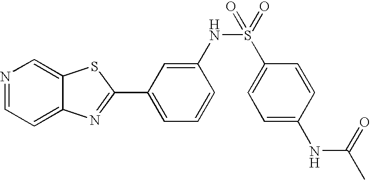 | | A | B |
| 289 | 427.5 | 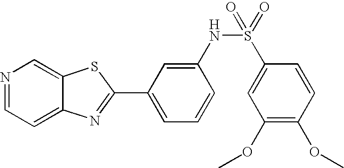 | | A | B |
| 290 | 445.5 | 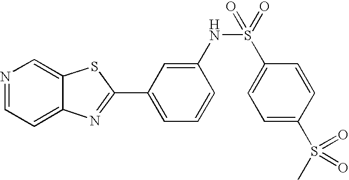 | | NA | C |
| 292 | 420.1 | 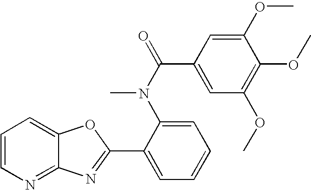 | | D | |
| 293 | 404.1 | 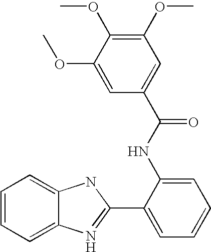 | | A′ | A |
| 294 | 374 | 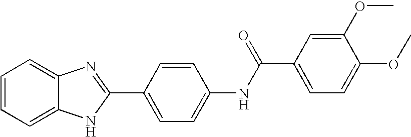 | | A′ | B |
| 295 | 252.1 | 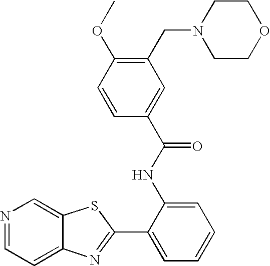 | | A′ | B |
| 296 | 376 | 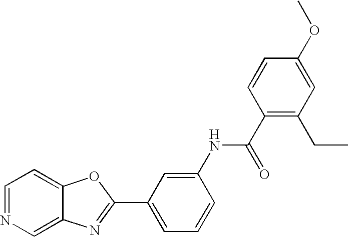 | | A | B |
| 297 | 376 | 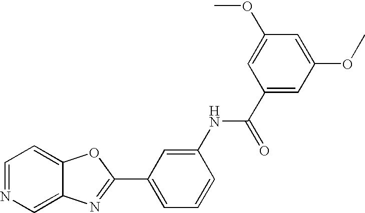 | | A | B |
| 298 | 406 | 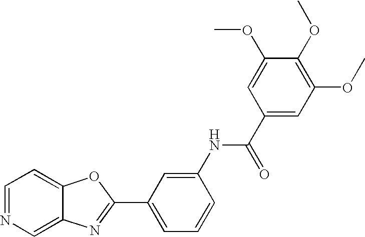 | | A | B |
| 299 | 359 | 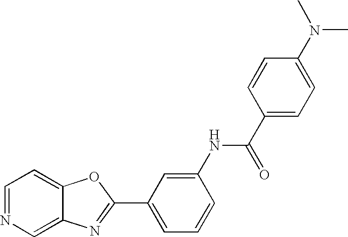 | | A | B |
| 303 | 437.5 | 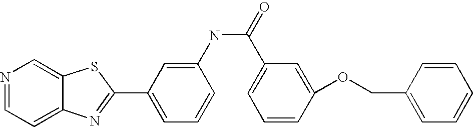 | | NA | |
| 304 | 336.4 | 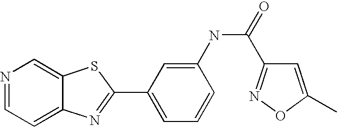 | | A′ | B |
| 305 | 414.5 | 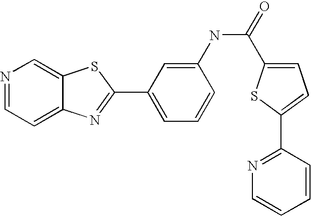 | | NA | |
| 306 | 424.5 | 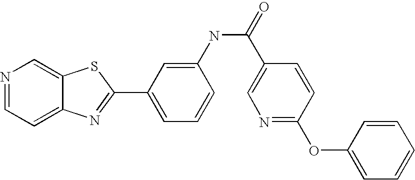 | | A | B |
| 307 | 382.4 | 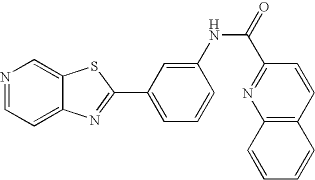 | | A′ | A |
| 308 | 400.3 | 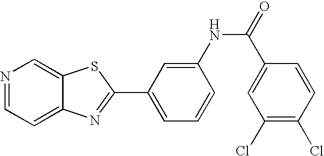 | | NA | |
| 309 | 367.4 | 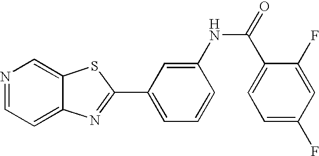 | | NA | |
| 310 | 387.5 | 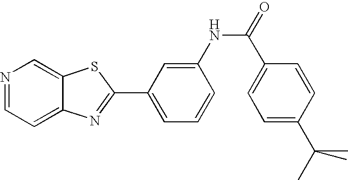 | | NA | |
| 311 | 359 | 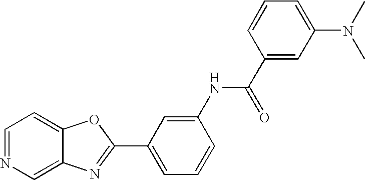 | | A | B |
| 313 | 418.1 | 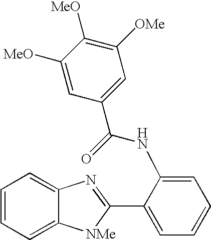 | | A | B |
| 314 | 388.1 | 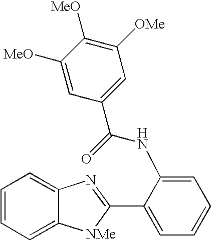 | | A | B |
| 315 | 388.1 | 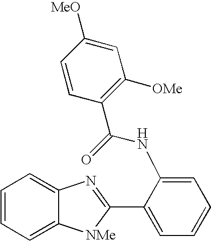 | | NA | |
| 317 | 392 | 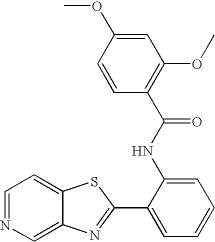 | | A′ | B |
| 318 | 392 | 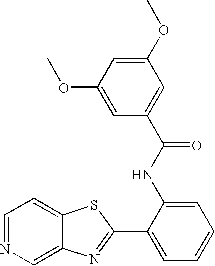 | | A | B |
| 319 | 422 | 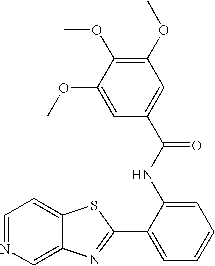 | | A′ | B |
| 320 | 375 | 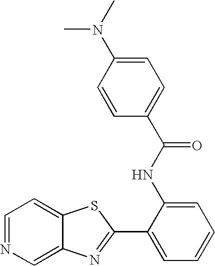 | | A′ | B |
| 321 | 375 | 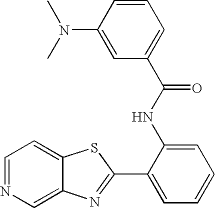 | | A′ | B |
| 322 | 392 | 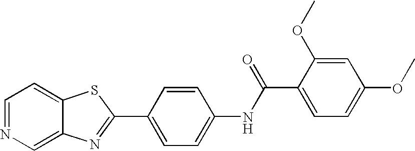 | | NA | |
| 323 | 392 | 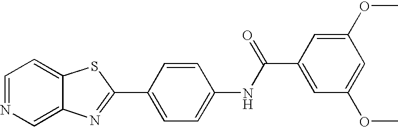 | | A | B |
| 324 | 422 | 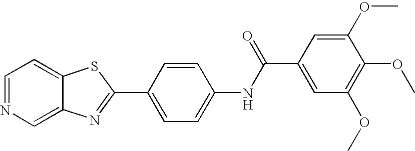 | | NA | |
| 325 | 375 | 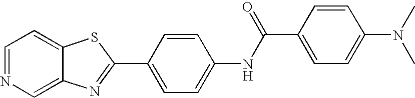 | | NA | |
| 326 | 478 | 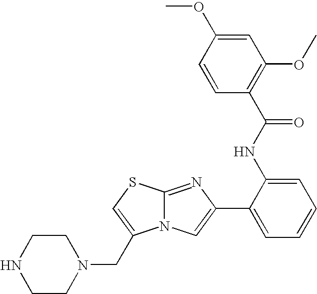 | | A | B |
| 327 | 461 | 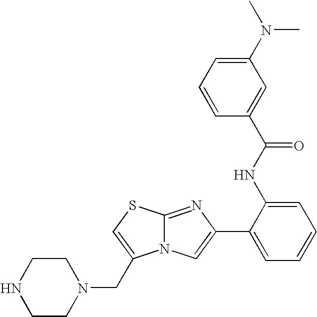 | | A | A |
| 328 | 418 | 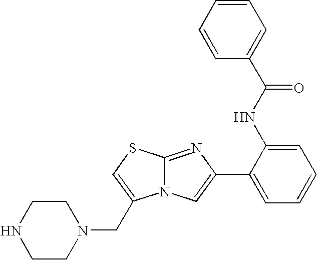 | | B | A |
| 329 | 462 | 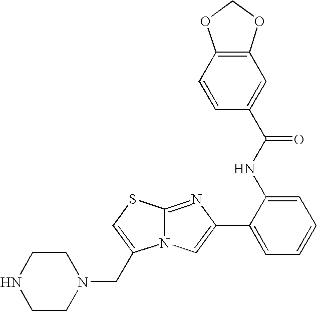 | | A | A |
| 330 | 443 | 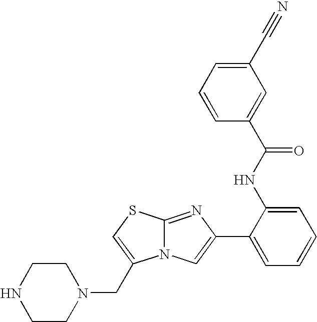 | | A | A |
| 331 | 335 | 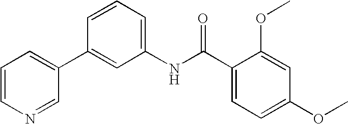 | | A | B |
| 332 | 335.1 | 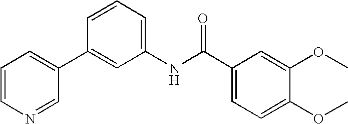 | | A | B |
| 333 | 365 | 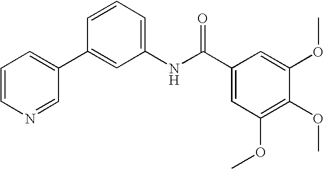 | | A | B |
| 334 | 340.1 | 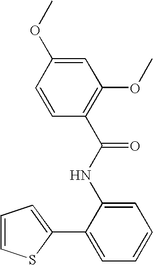 | | A | B |
| 335 | 340 | 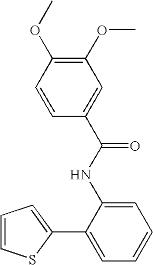 | | A | B |
| 336 10 | 370 | 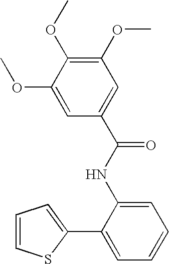 | | A | B |
| 337 | 443 | 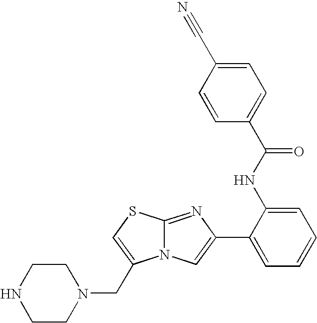 | | NA | |
| 338 | 458 | 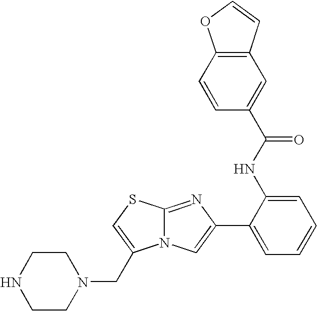 | | A | A |
| 339 | 376 | 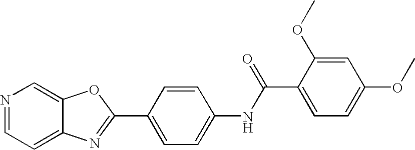 | | NA | |
| 340 | 376 | 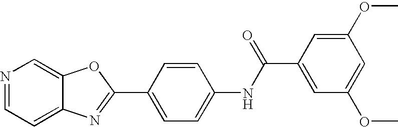 | | NA | |
| 341 | 406 | 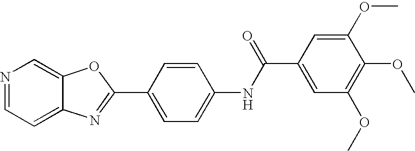 | | A′ | B |
| 342 | 359 | 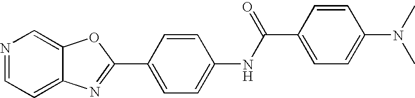 | | NA | |
| 343 | 406 | 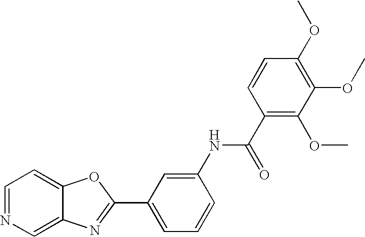 | | A | B |
| 344 | 375 | 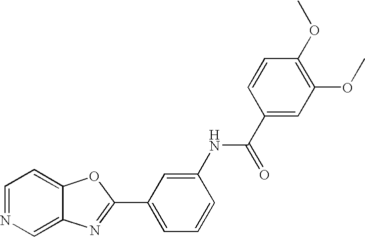 | | A′ | B |
| 345 | 376 | 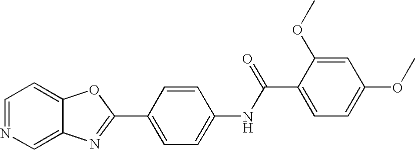 | | NA | |
| 346 | 376 | 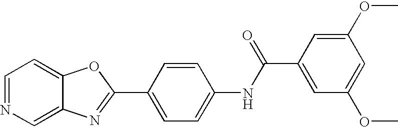 | | NA | |
| 347 | 406 | 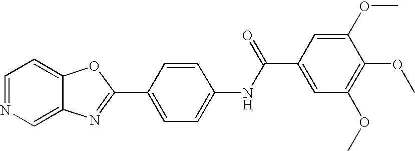 | | A′ | B |
| 348 | 359 | 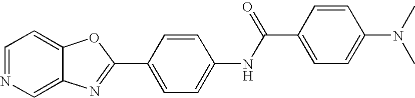 | | NA | |
| 349 | 375 | 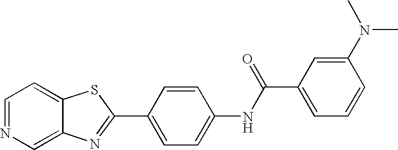 | | NA | |
| 350 | 431.1 | 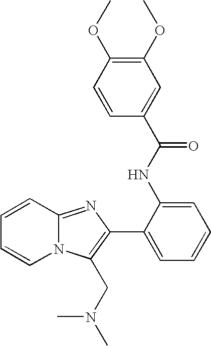 | | B | B |
| 351 | 461 | 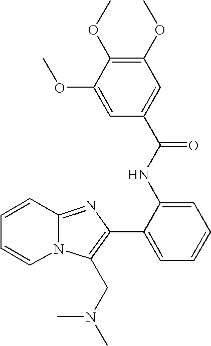 | | A | B |
| 359 | 472.1 | 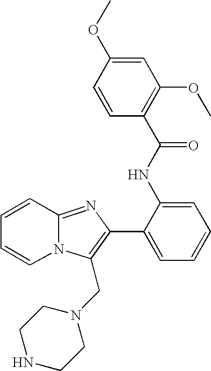 | | NA | |
| 362 | 502 | 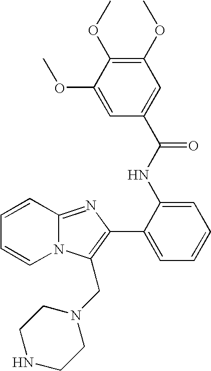 | | B | B |
| 364 | 472 | 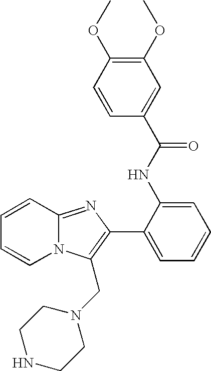 | | D | A |
| 367 | 359.1 | 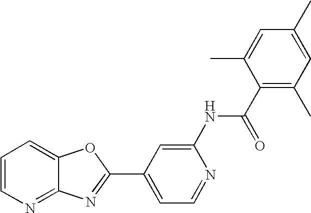 | | NA | |
| 369 | 350.8 | 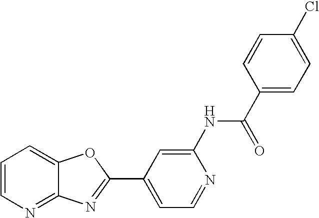 | | NA | |
| 370 | 437.0 | 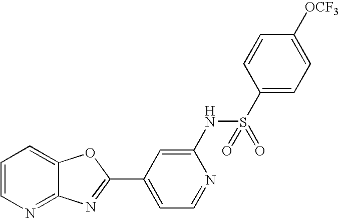 | | NA | |
| 371 | 381.1 | 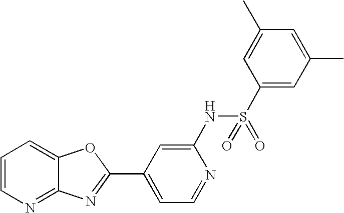 | | NA | |
| 372 | 431.1 | 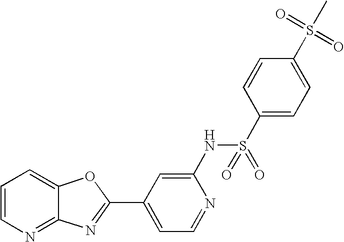 | | NA | |
| 373 | 445.0 | 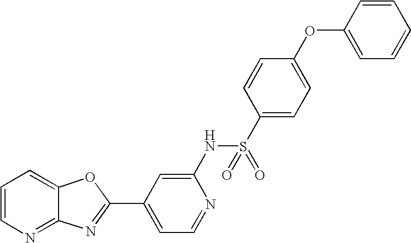 | | NA | |
| 374 | 421.1 | 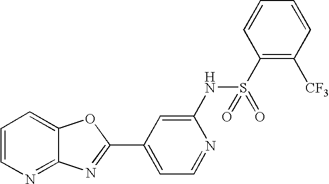 | | NA | |
| 375 | 358.2 | 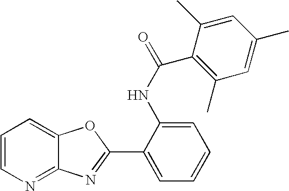 | | NA | |
| 376 | 350.0 | 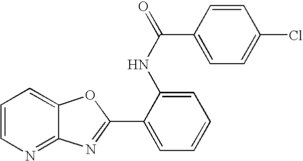 | | NA | |
| 377 | | 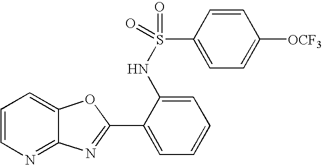 | | NA | |
| 378 | 412.1 | 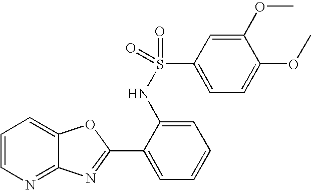 | | NA | |
| 379 | 380.0 | 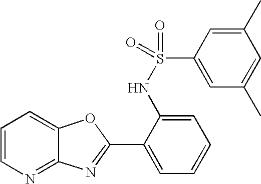 | | NA | |
| 380 | 429.9 | 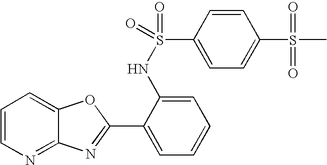 | | NA | |
| 381 | 444.1 | 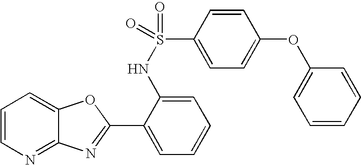 | | NA | |
| 382 | 420.0 | 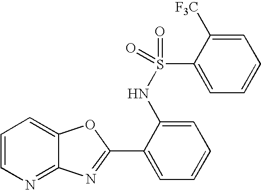 | | NA | |
| 383 | 515.7 | 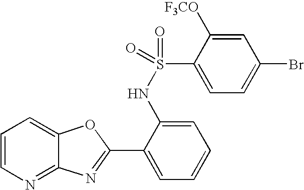 | | NA | |
| 384 | 487.8 | 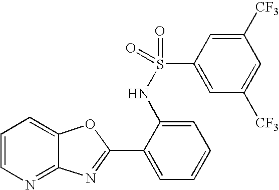 | | NA | |
| 385 | 397.2 | 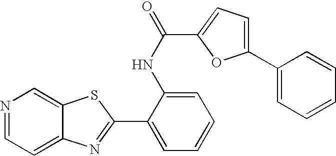 | | NA | |
| 387 | 366.8 | 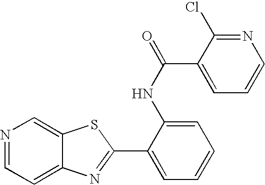 | | NA | |
| 390 | 412.5 | 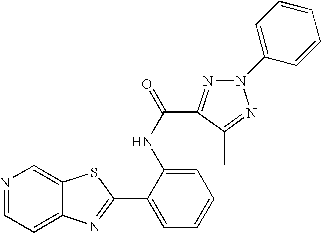 | | A | B |
| 391 | 333.4 | 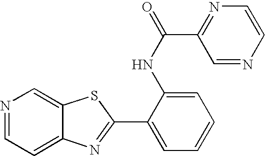 | | A | B |
| 392 | 424.5 | 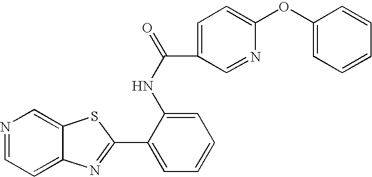 | | A | B |
| 393 | 400.3 | 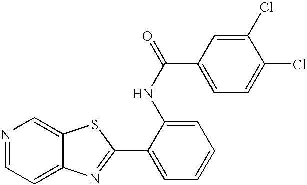 | | NA | |
| 394 | 457.6 | 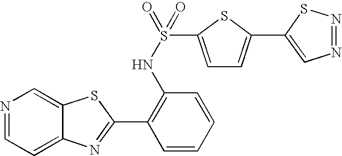 | | NA | |
| 396 | 389.5 | 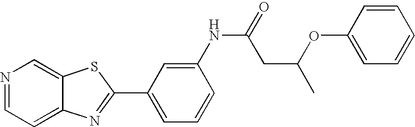 | | A | B |
| 398 | 460.1 | 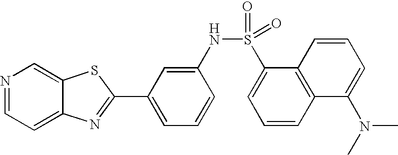 | | A | B |
| 399 | 424.5 | 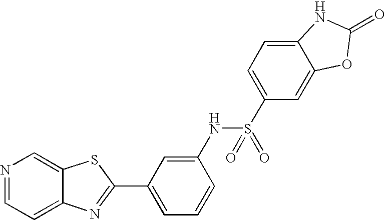 | | A | B |
| 400 | 392 | 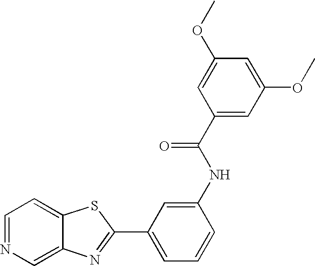 | | NA | |
| 401 | 422 | 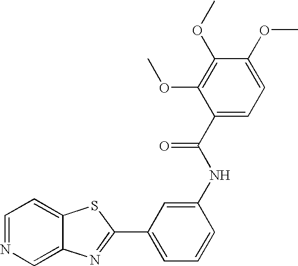 | | A | B |
| 402 | 375 | 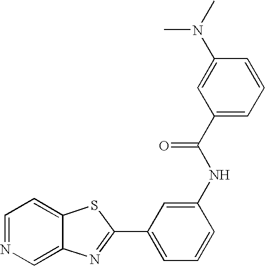 | | A′ | B |
| 403 | 375 | 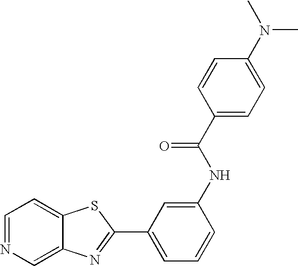 | | A′ | B |
| 404 | 376 | 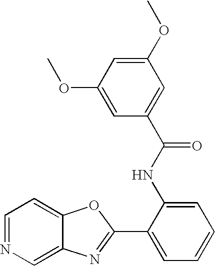 | | A′ | B |
| 405 | 406 | 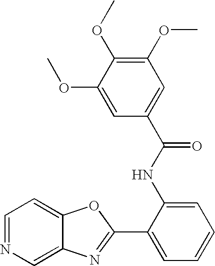 | | A′ | B |
| 406 | 359 | 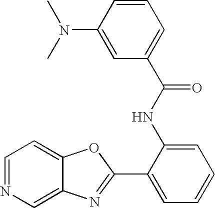 | | A′ | B |
| 407 | 359 | 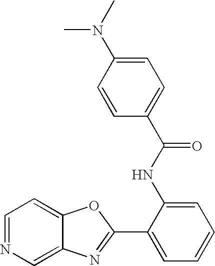 | | A′ | B |
| 408 | 359 | 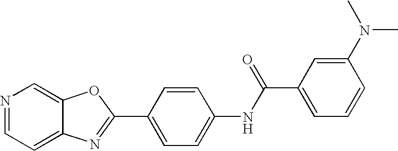 | | B | B |
| 409 | 422 | 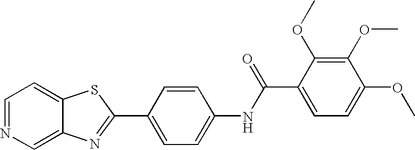 | | NA | |
| 410 | 359 | 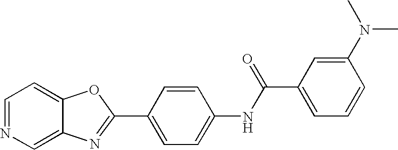 | | NA | |
| 411 | 422 | 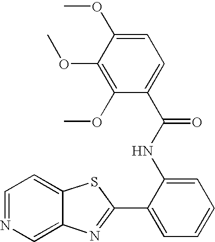 | | A | B |
| 412 | 406 | 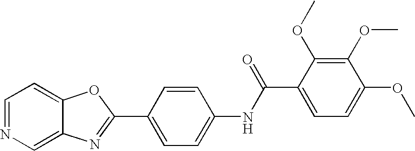 | | NA | |
| 413 | 385.1 | 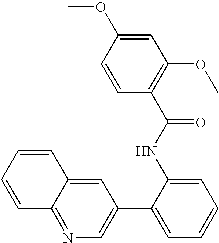 | | B | B |
| 414 | 385 | 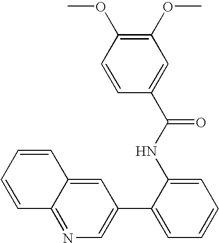 | | A | B |
| 415 | 415 | 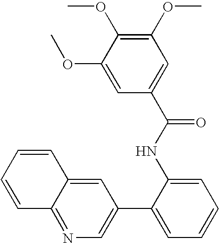 | | B | B |
| 416 | 368 | 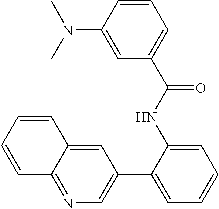 | | NA | |
| 419 | 406 | 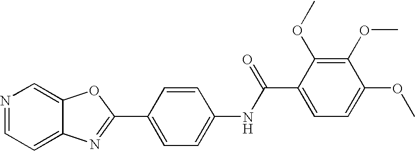 | | NA | |
| 420 | 376 | 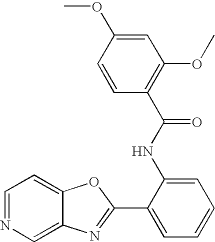 | | NA | |
| 421 | 406 | 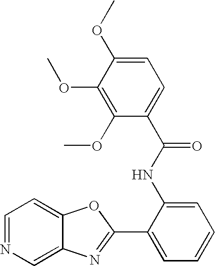 | | A | B |
| 422 | 382 | 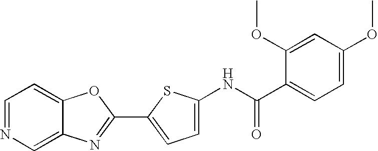 | | A | B |
| 423 | 382 | 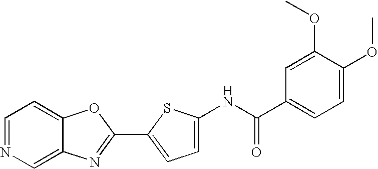 | | A′ | B |
| 424 | 382 | 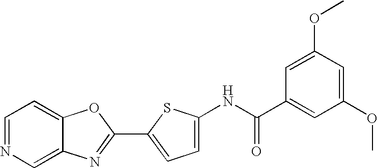 | | NA | |
| 425 | 412 | 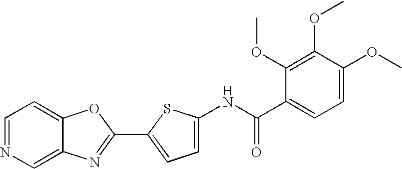 | | NA | |
| 426 | 412 | 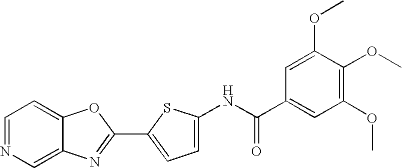 | | A′ | B |
| 427 | 365 | 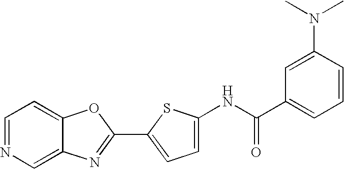 | | A′ | B |
| 428 | 365 | 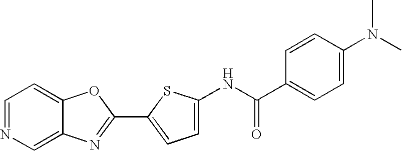 | | NA | |
| 429 | 376 | 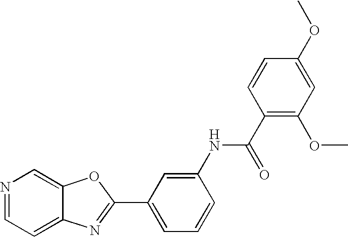 | | A′ | B |
| 430 | 406 | 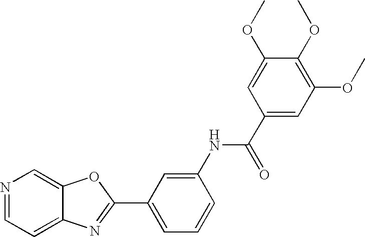 | | A | A |
| 431 | 359 | 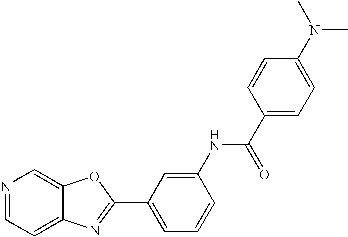 | | A | B |
| 436 | 445.1 | 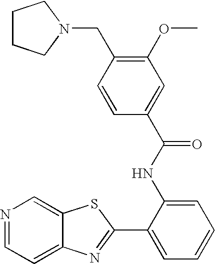 | | A | A |
| 437 | 375.1 | 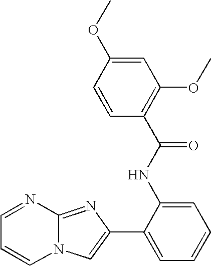 | | A | B |
| 438 | 375 | 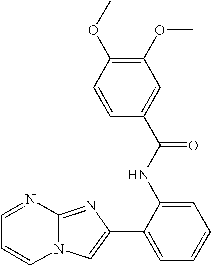 | | A′ | A |
| 439 | 405 | 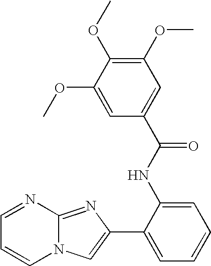 | | A′ | B |
| 440 | 468 | 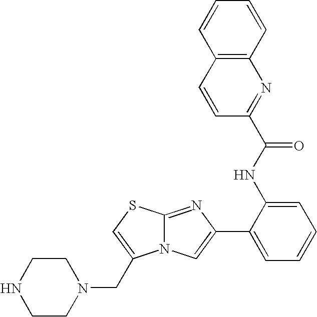 | | A′ | A |
| 441 | 470 | 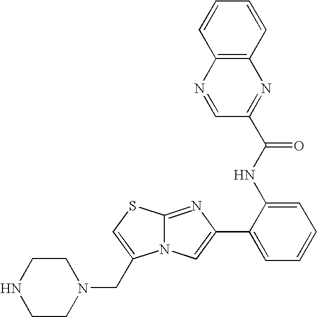 | | A′ | A |
| 442 | 472 | 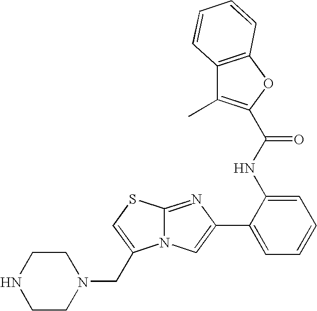 | | A′ | A |
| 443 | 436 | 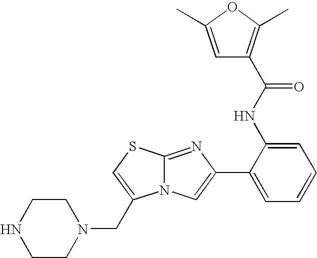 | | A | A |
| 444 | 464 | 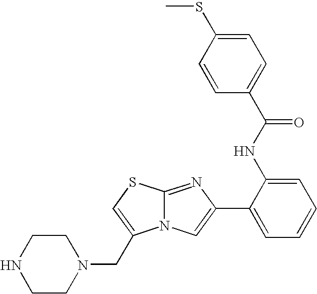 | | A | A |
| 445 | 432 | 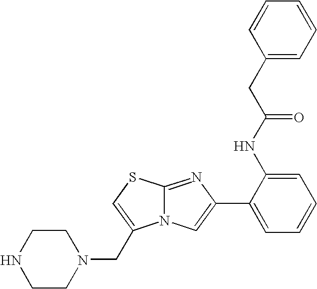 | | A | B |
| 446 | 424 | 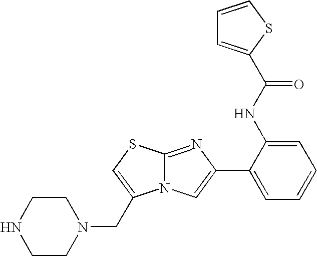 | | A | B |
| 447 | 484 | 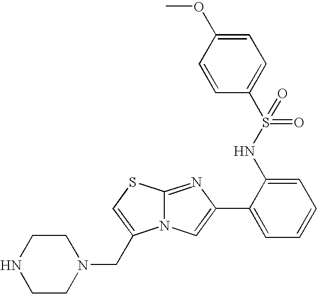 | | NA | |
| 448 | 510 | 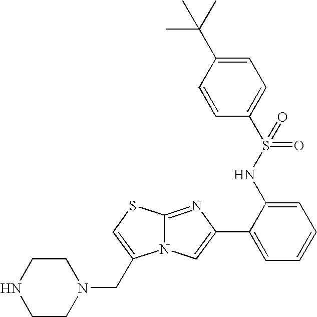 | | NA | |
| 449 | 376 | 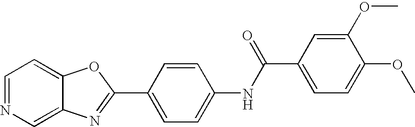 | | NA | |
| 450 | 392 | 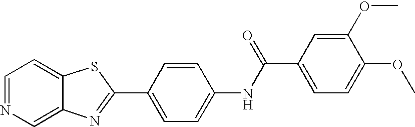 | | NA | |
| 451 | 376 | 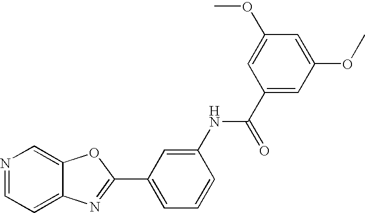 | | A | B |
| 452 | 406 | 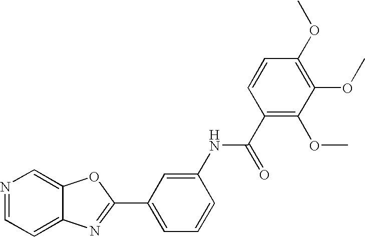 | | A | B |
| 453 | 359 | 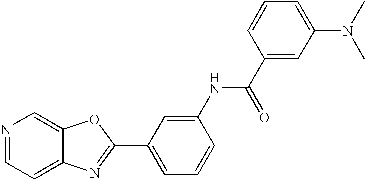 | | A | B |
| 454 | 376 | 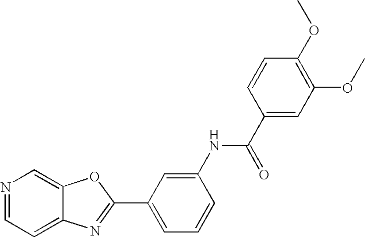 | | A | B |
| 455 | 376 | 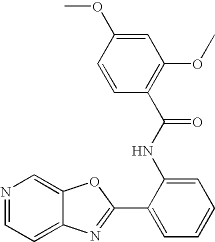 | | A′ | B |
| 456 | 376 | 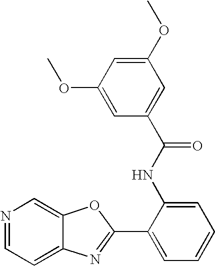 | | A′ | B |
| 457 | 406 | 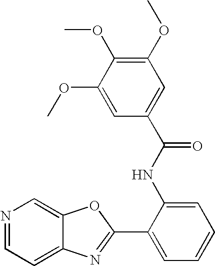 | | A | B |
| 458 | 359 | 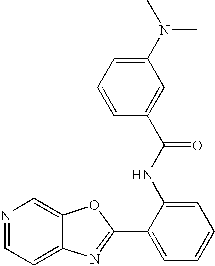 | | A′ | B |
| 459 | 359 | 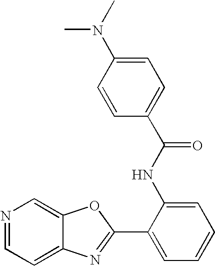 | | A′ | B |
| 460 | 359.4 | 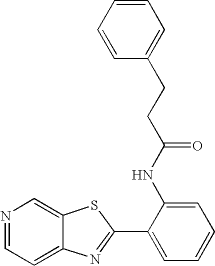 | | A | B |
| 461 | 367.4 | 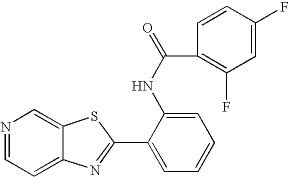 | | NA | |
| 462 | 391.5 | 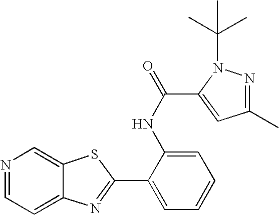 | | A′ | B |
| 463 | 375.4 | 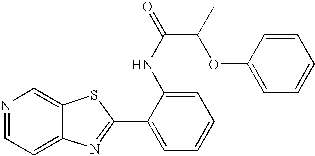 | | A | B |
| 465 | 395.9 | 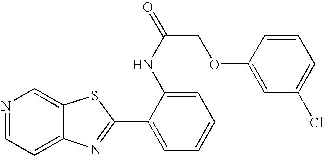 | | NA | |
| 466 | 445.5 | 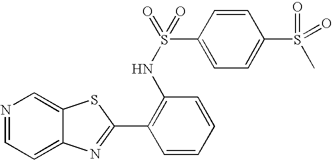 | | NA | |
| 467 | 427.2 | 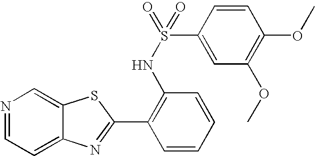 | | NA | |
| 468 | 345.0 | 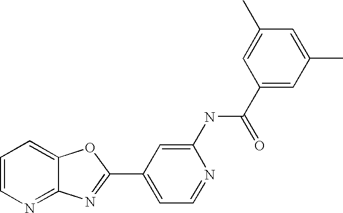 | | A | B |
| 469 | 435.4 | 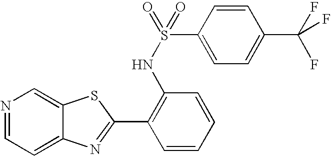 | | NA | |
| 470 | 365.0 | 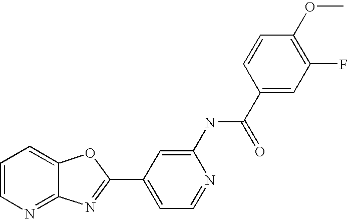 | | NA | |
| 471 | 488.9 | 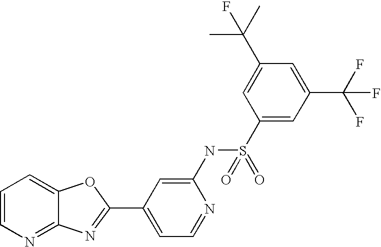 | | NA | |
| 472 | 437.5 | 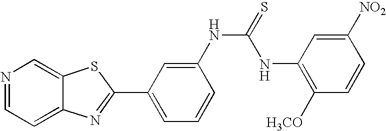 | | NA | |
| 473 | 420.5 | 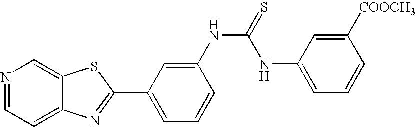 | | A′ | B |
| 474 | | 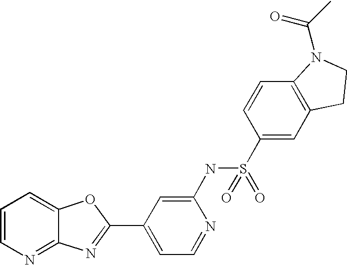 | | NA | |
| 475 | 408.6 | 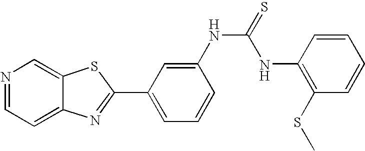 | | NA | |
| 476 | 410.9 | 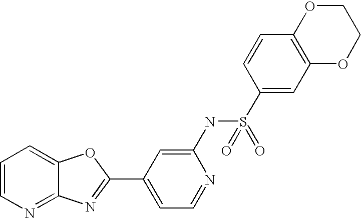 | | NA | |
| 477 | 435.9 | 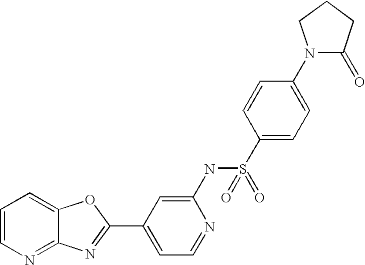 | | NA | |
| 478 | 404.8 | 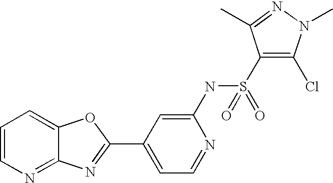 | | NA | |
| 479 | 420.9 | 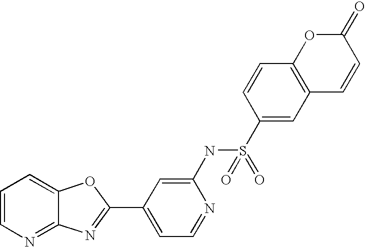 | | NA | |
| 481 | 428.5 | 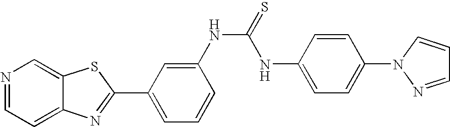 | | A | B |
| 482 | 344.1 | 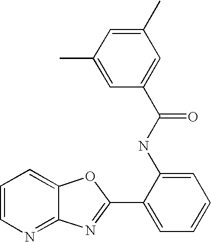 | | A′ | B |
| 483 | 364.1 | 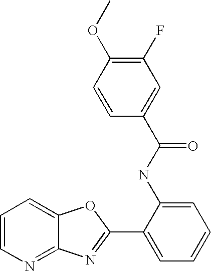 | | NA | |
| 484 | 448.6 | 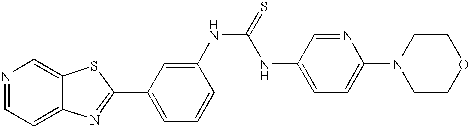 | | A | B |
| 485 | 363.5 | 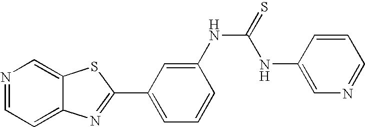 | | A | A |
| 486 | 385.9 | 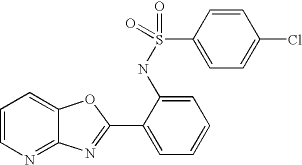 | | NA | |
| 487 | 396.1 | 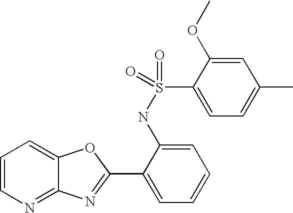 | | NA | |
| 488 | 435.1 | 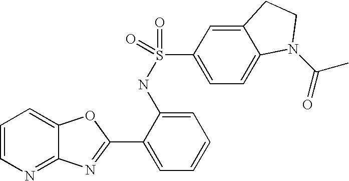 | | NA | |
| 489 | 410.1 | 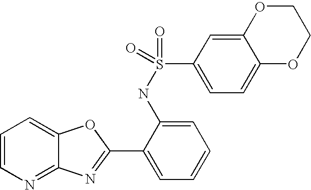 | | NA | |
| 490 | 434.9 | 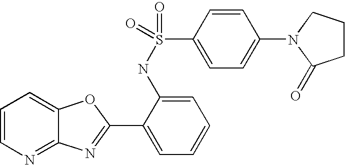 | | NA | |
| 491 | 420.5 | 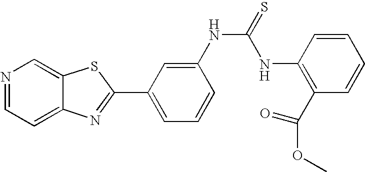 | | NA | |
| 492 | 404.0 | 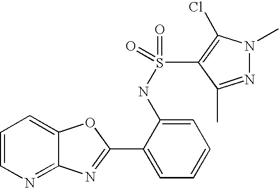 | | NA | |
| 493 | | 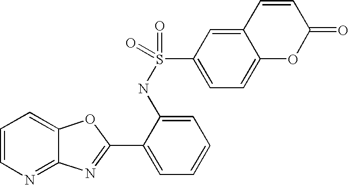 | | NA | |
| 494 | 422.9 | 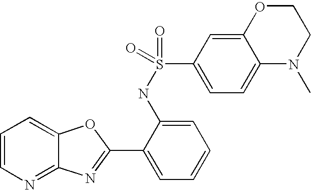 | | NA | |
| 495 | 366.0 | 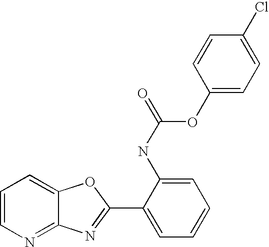 | | NA | |
| 496 | 349.9 | 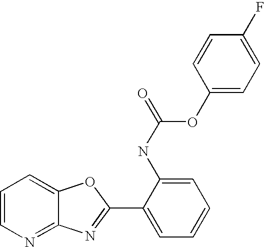 | | NA | |
| 497 | 346.0 | 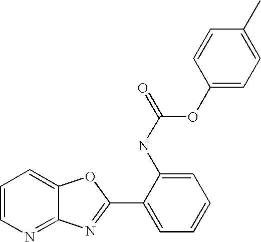 | | NA | |
| 498 | 406.5 | 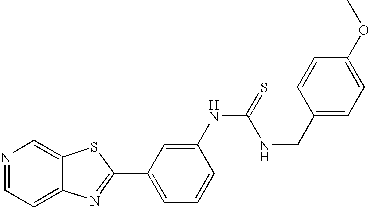 | | A | B |
| 499 | 362.1 | 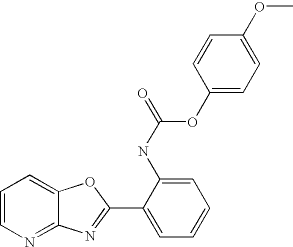 | | NA | |
| 500 | | 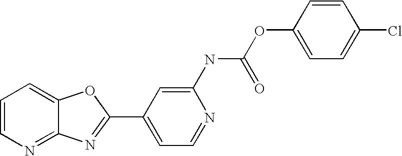 | | NA | |
| 501 | 347.1 | 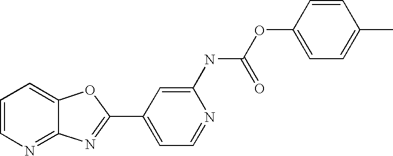 | | NA | |
| 502 | 363.1 | 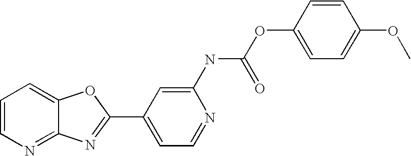 | | NA | |
| 503 | 443.1 | 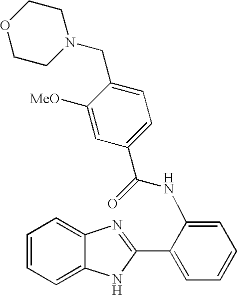 | | A′ | A |
| 504 | 358 | 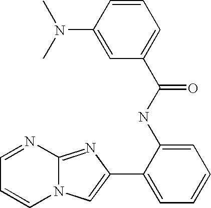 | | A | B |
| 505 | 359 | 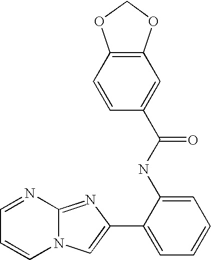 | | NA | |
| 506 | 429.1 | 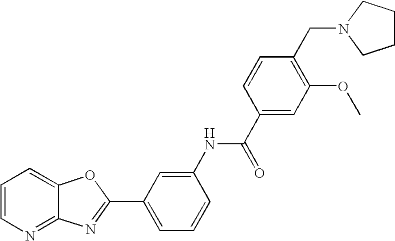 | | A | A |
| 507 | 388.1 | 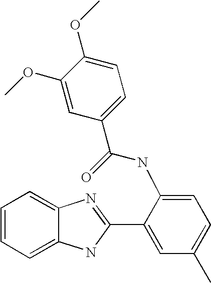 | | A′ | A |
| 508 | 366.1 | 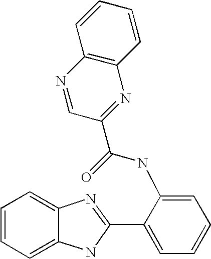 | | A′ | A |
| 510 | 457 | 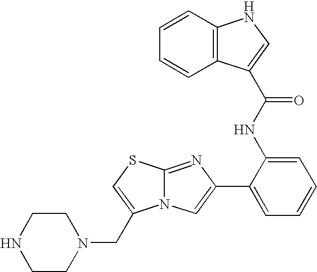 | | A′ | A |
| 511 | 460 | 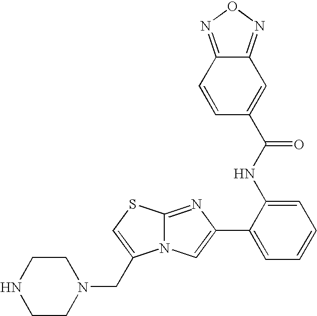 | | NA | |
| 512 | 484 | 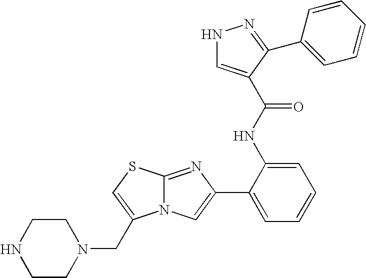 | | A′ | A |
| 513 | 470 | 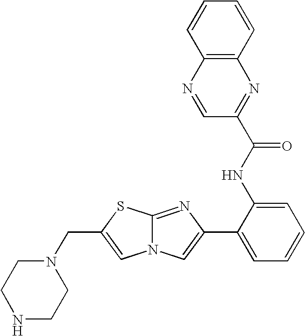 | | A′ | A |
| 514 | 466 | 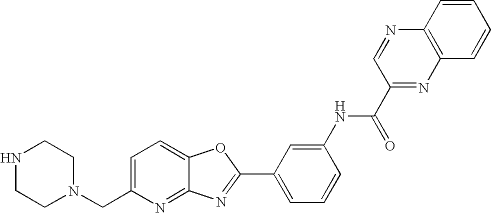 | | A | B |
| 515 | 398 | 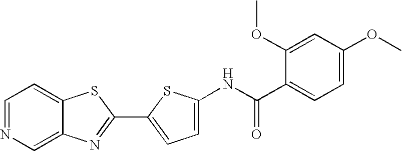 | | A′ | B |
| 516 | 398 | 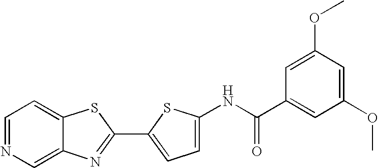 | | NA | |
| 517 | 428 | 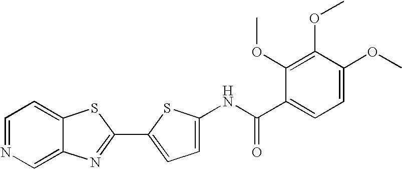 | | NA | |
| 518 | 381 | 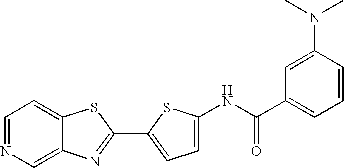 | | A′ | B |
| 519 | 381 | 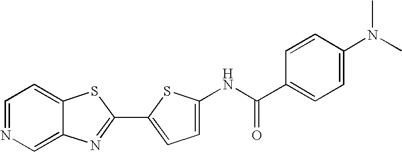 | | A′B | |
| 520 | 428 | 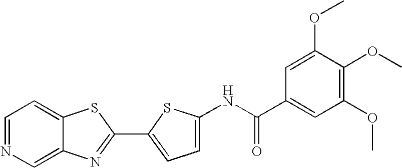 | | A′ | B |
| 521 | 375.0 | 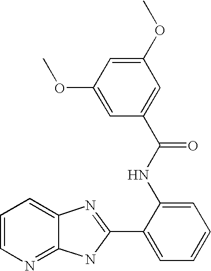 | | A′ | B |
| 522 | 405.0 | 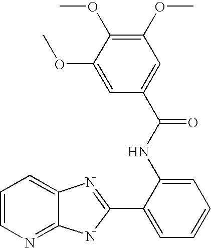 | | A | A |
| 523 | 359.0 | 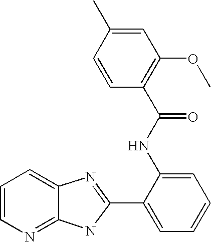 | | A | B |
| 524 | 358.0 | 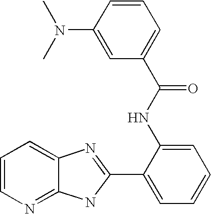 | | A′ | B |
| 525 | 375.0 | 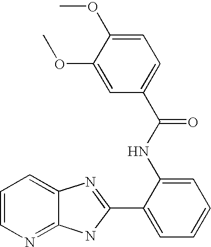 | | A′ | B |
| 526 | 400.0 | 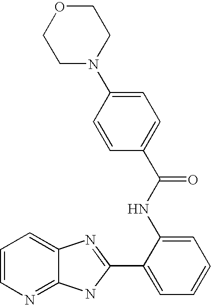 | | A′ | A |
| 527 | 398.0 | 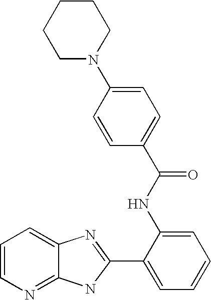 | | A′ | B |
| 528 | 412.9 | 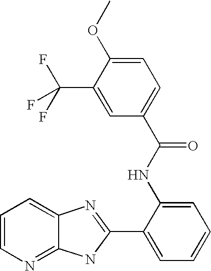 | | A′ | B |
| 529 | 399.1 | 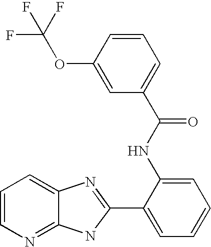 | | A′ | B |
| 530 | 359.0 | 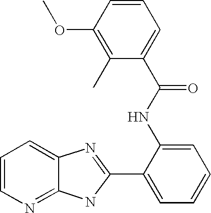 | | A′ | B |
| 531 | 345.0 | 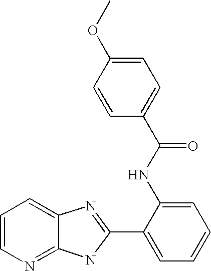 | | A′ | B |
| 532 | 345.0 | 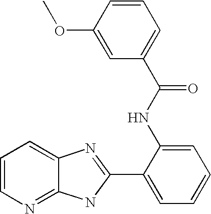 | | A′ | B |
| 533 | 315.0 | 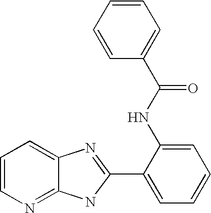 | | A′ | B |
| 534 | 358.0 | 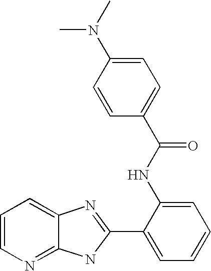 | | A′ | B |
| 535 | 428.1 | 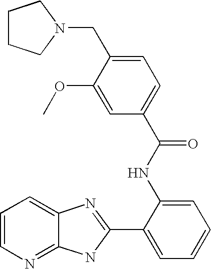 | | A | A |
| 536 | 442.1 | 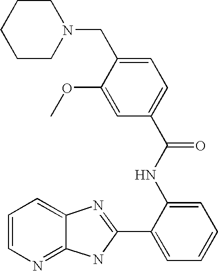 | | A | A |
| 537 | 444.0 | 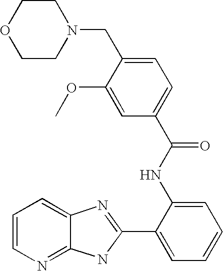 | | A′ | A |
| 538 | 443.1 | 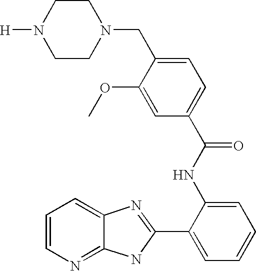 | | A | A |
| 539 | 457.1 | 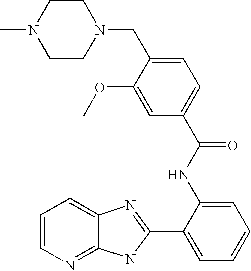 | | A′ | A |
| 540 | 402.1 | 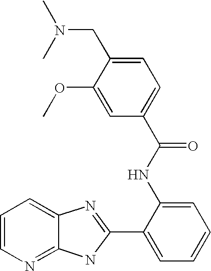 | | A | A |
| 541 | 443.1 | 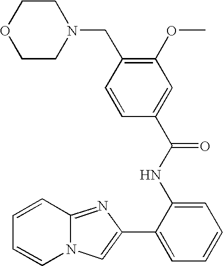 | | A | B |
| 542 | 444 | 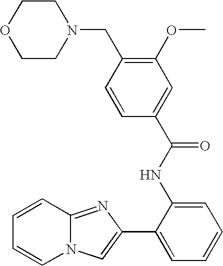 | | A | A |
| 543 | 420 | 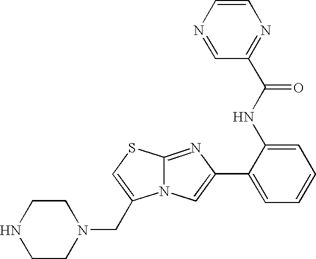 | | A′ | A |
| 544 | 474 | 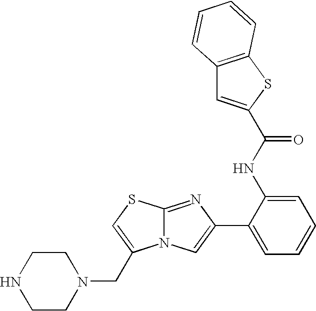 | | A | A |
| 545 | 378.1 | 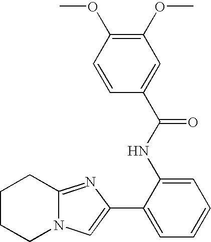 | | A | B |
| 546 | 408 | 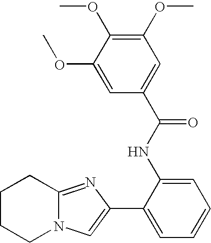 | | A | B |
| 547 | 369 | 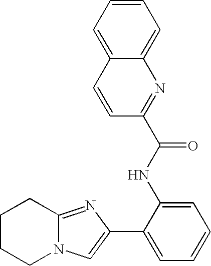 | | A′ | B |
| 548 | 370 | 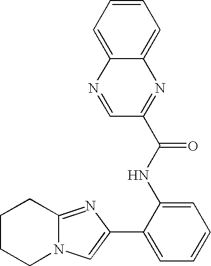 | | A′ | B |
| 556 | 365.1 | 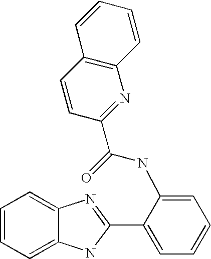 | | A′ | A |
| 557 | 542.1 | 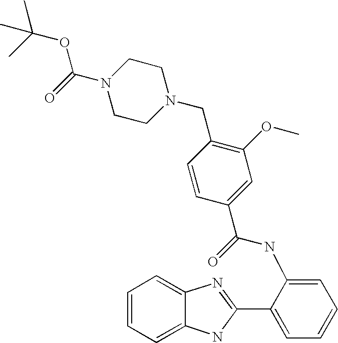 | | NA | |
| 558 | 442.1 | 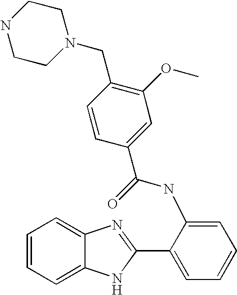 | | A′ | A |
| 559 | 508 | 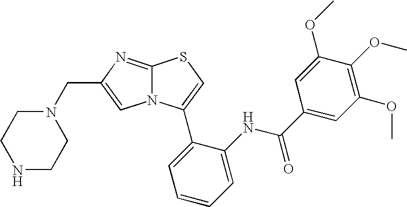 | | NA | |
| 560 | 470 | 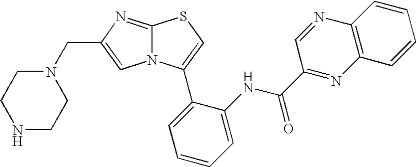 | | NA | |
| 561 | 382 | 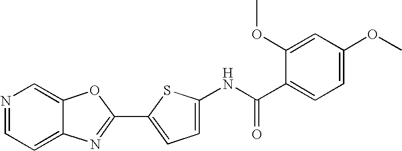 | | A′ | B |
| 562 | 382 | 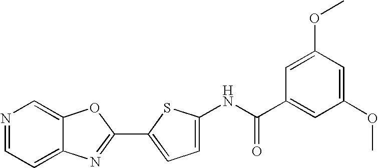 | | A′ | B |
| 563 | 412 | 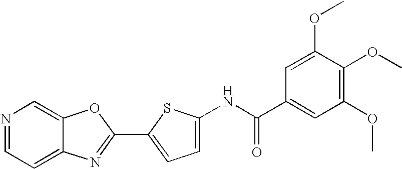 | | A | B |
| 565 | 400.8 | 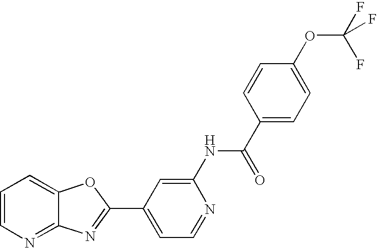 | | NA | |
| 566 | 409.9 | 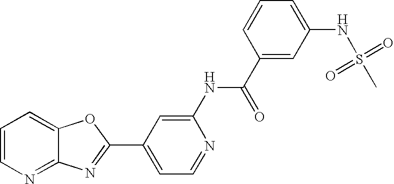 | | A | B |
| 567 | 374.9 | 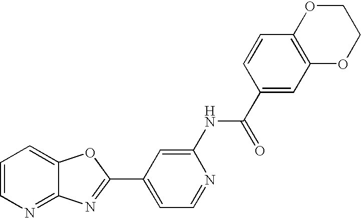 | | NA | |
| 568 | 367.0 | 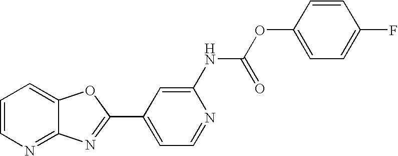 | | NA | |
| 569 | 374.9 | 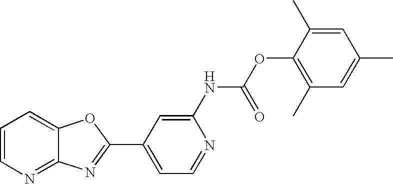 | | NA | |
| 570 | 361.0 | 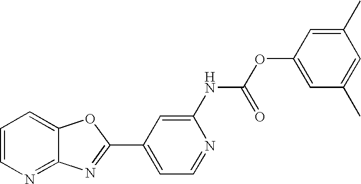 | | NA | |
| 571 | 444.0 | 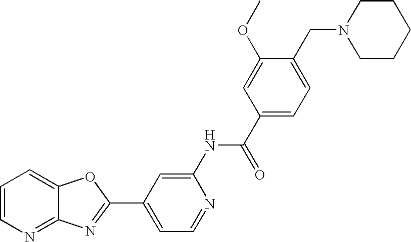 | | A | B |
| 572 | 429.9 | 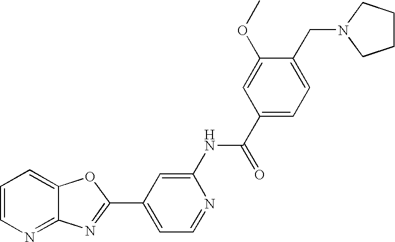 | | B | B |
| 573 | 431.8 | 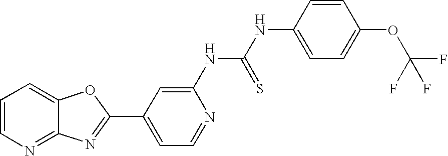 | | NA | |
| 574 | 376.2 | 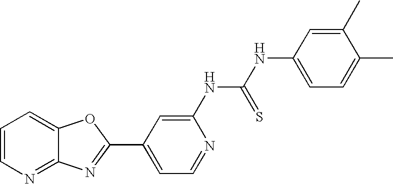 | | NA | |
| 575 | 439.9 | 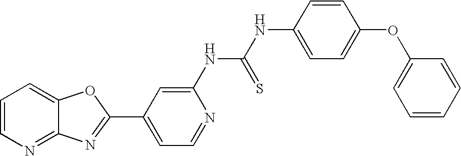 | | NA | |
| 576 | 376.1 | 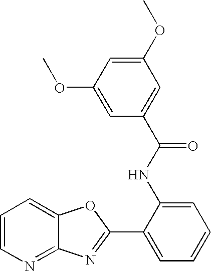 | | NA | |
| 577 | 400.1 | 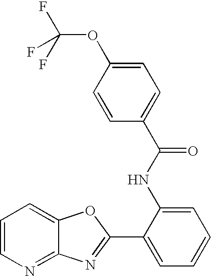 | | NA | |
| 578 | 368.1 | 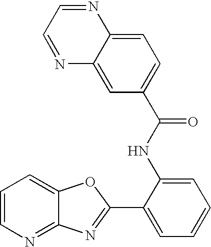 | | NA | |
| 579 | 401.1 | 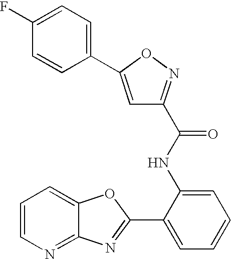 | | NA | |
| 580 | 374.0 | 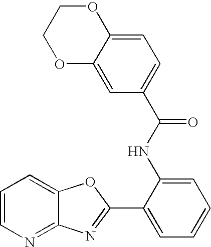 | | NA | |
| 581 | 334.0 | 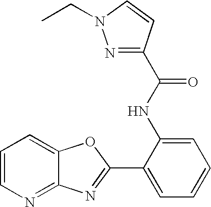 | | A | B |
| 582 | 400.0 | 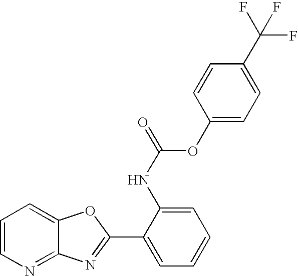 | | NA | |
| 583 | 374.1 | 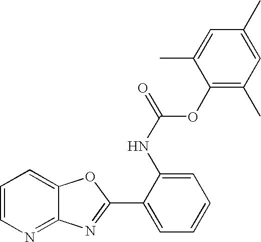 | | NA | |
| 584 | 360.0 | 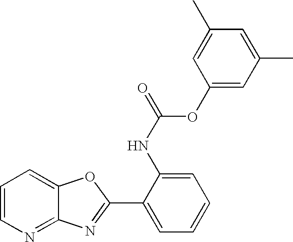 | | NA | |
| 585 | 443.1 | 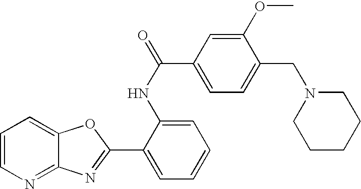 | | A | B |
| 587 | 427.1 | 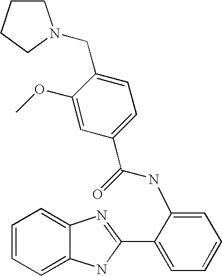 | | A′ | B |
| 588 | 520 | 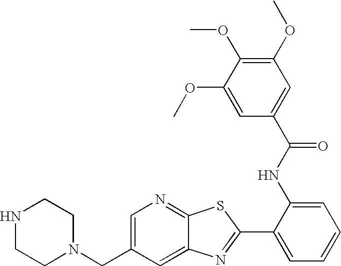 | | A | A |
| 589 | 490 | 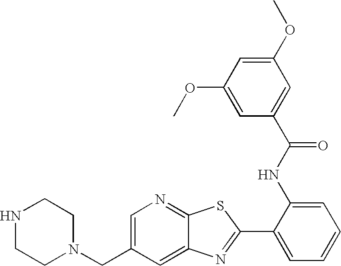 | | A′ | A |
| 590 | 474 | 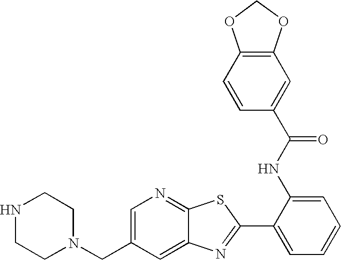 | | A | B |
| 591 | 458 | 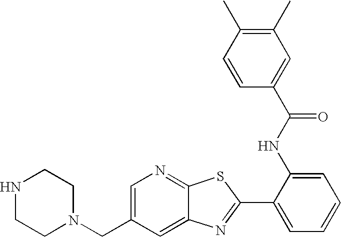 | | A′ | A |
| 592 | 455 | 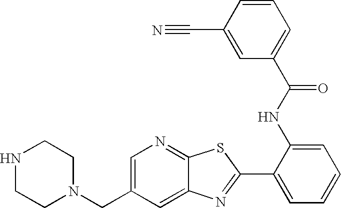 | | A′ | B |
| 593 | 473 | 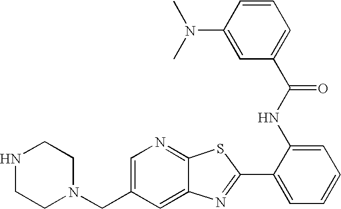 | | A′ | A |
| 594 | 466 | 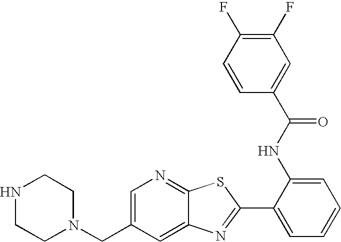 | | A | B |
| 596 | 467.4 | 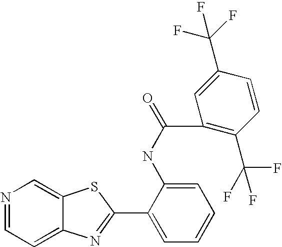 | | A′ | B |
| 597 | 377.2 | 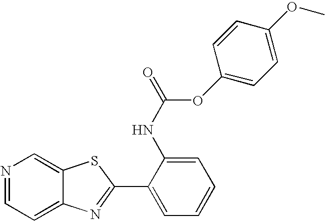 | | A′ | B |
| 599 | 422.3 | 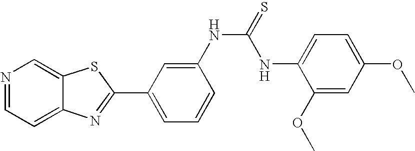 | | A′ | B |
| 600 | 422.5 | 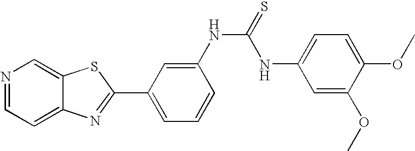 | | D | B |
| 601 | 405.5 | 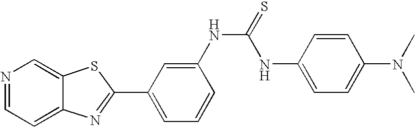 | | NA | |
| 604 | 360.4 | 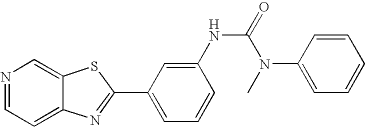 | | NA | |
| 605 | 377.4 | 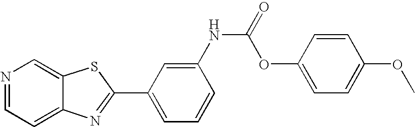 | | NA | |
| 607 | 415.4 | 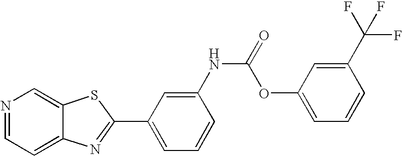 | | NA | |
| 608 | 332.4 | 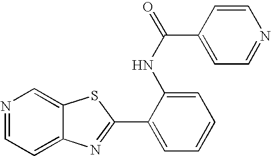 | | NA | |
| 609 | 439.3 | 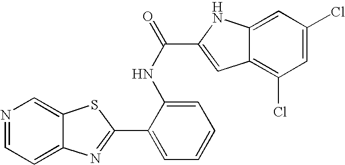 | | NA | |
| 610 | 418.6 | 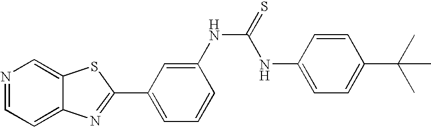 | | NA | |
| 611 | 465.6 | 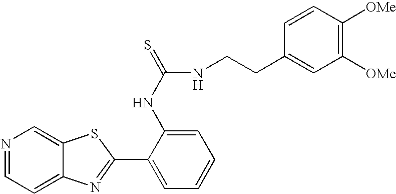 | | A′ | B |
| 612 | 402.5 | 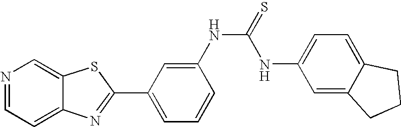 | | NA | |
| 614 | 428.5 | 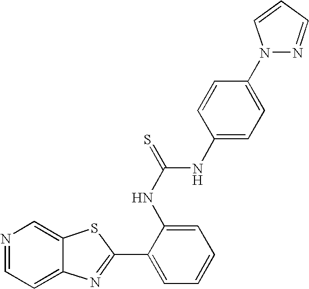 | | NA | |
| 615 | 408.6 | 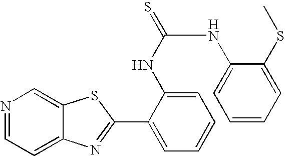 | | NA | |
| 616 | 437.5 | 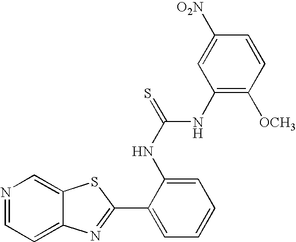 | | | |
| 617 | 471.1 | 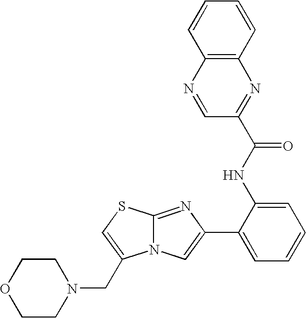 | | A′ | A |
| 618 | 469.1 | 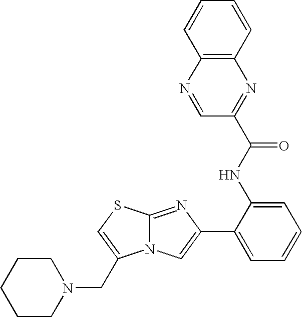 | | A′ | B |
| 619 | 455 | 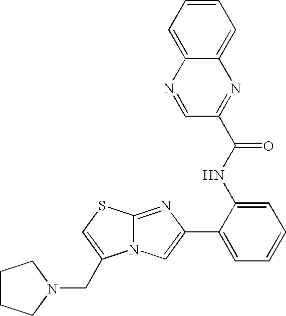 | | A′ | B |
| 620 | 493.1 | 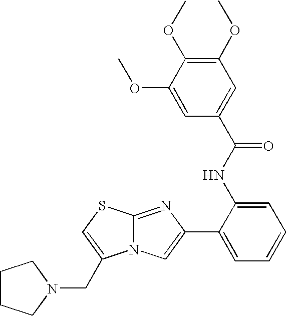 | | A′ | B |
| 621 | 472 | 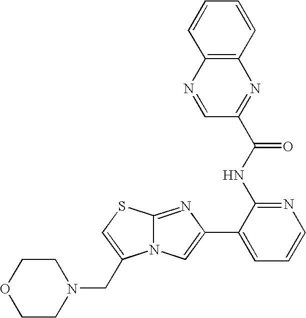 | | A′ | A |
| 622 | 482 | 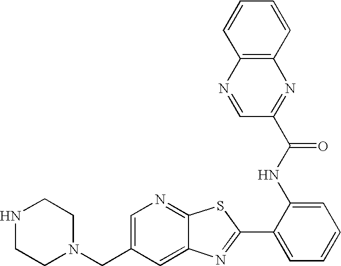 | | A′ | A |
| 623 | 437 | 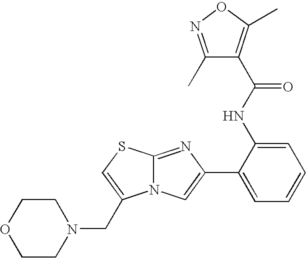 | | NA | C |
| 624 | 458 | 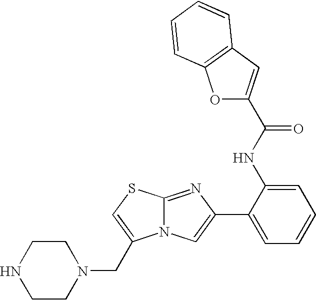 | | A′ | A |
| 625 | 496 | 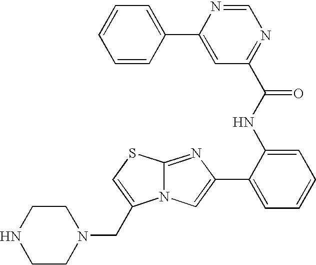 | | A′ | A |
| 628 | 574.1 | 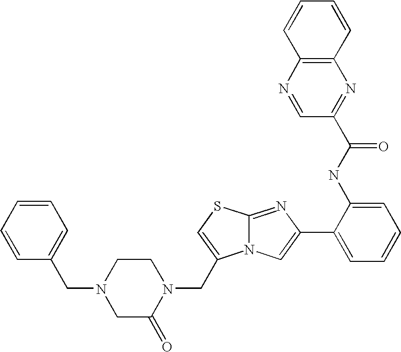 | | A′ | B |
| 629 | 429.0 | 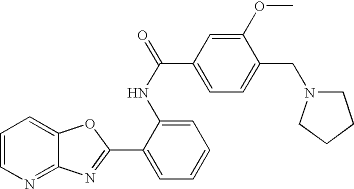 | | A | A |
| 630 | 403.1 | 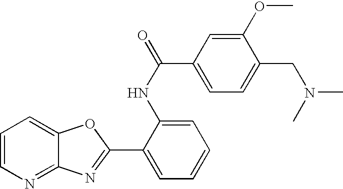 | | A | A |
| 631 | 445.0 | 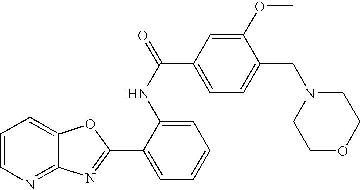 | | A′ | B |
| 632 | 458.1 | 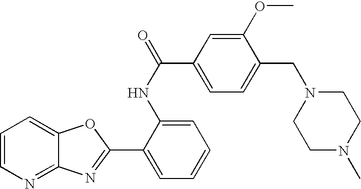 | | A | A |
| 633 | 423.3 | 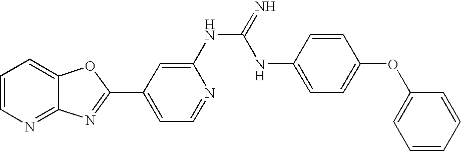 | | NA | C |
| 634 | 364.9 | 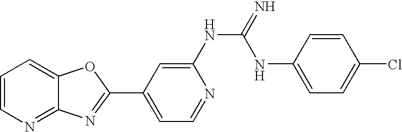 | | D | B |
| 635 | 421.1 | 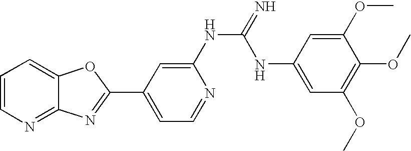 | | D | A |
| 636 | 359.9 | 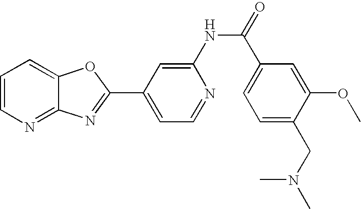 | | B | B |
| 637 | 459.3 | 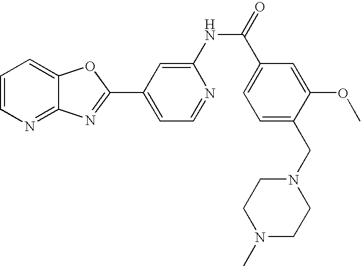 | | D | B |
| 638 | 445.2 | 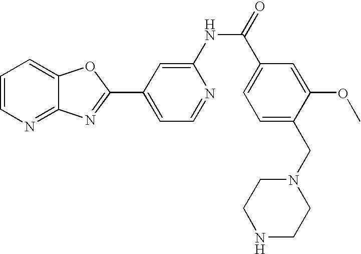 | | D | B |
| 639 | 378.9 | 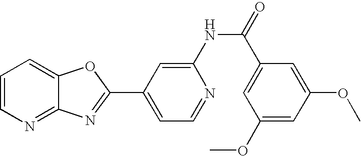 | | A | B |
| 640 | 368.9 | 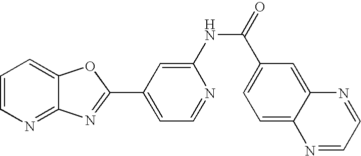 | | NA | C |
| 641 | 334.9 | 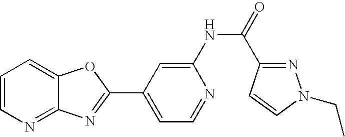 | | D | B |
| 642 | 446.2 | 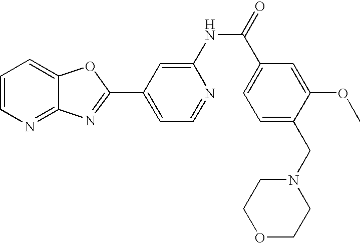 | | D | B |
| 643 | 535.1 | 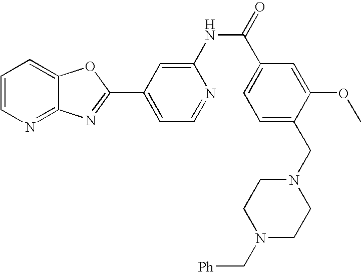 | | A | B |
| 644 | 434 | 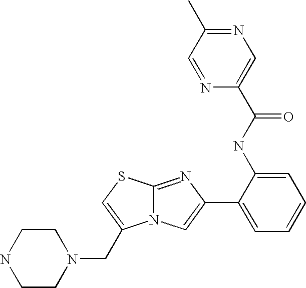 | | A | A |
| 645 | 469 | 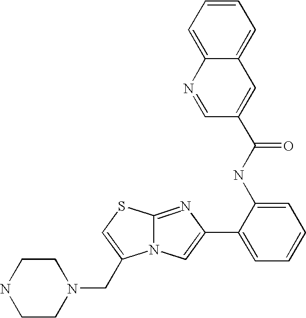 | | A′ | A |
| 646 | 481 | 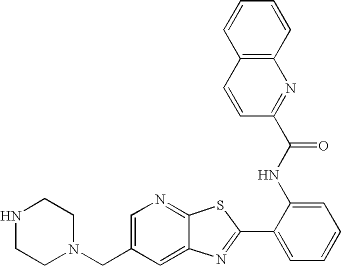 | | A | A |
| 647 | 473.1 | 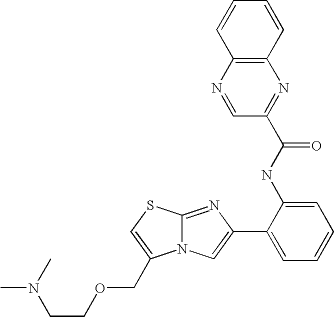 | | A′ | A |
| 648 | 402.1 | 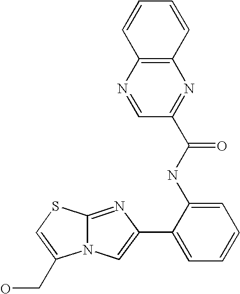 | | A′ | B |
| 649 | 512.2 | 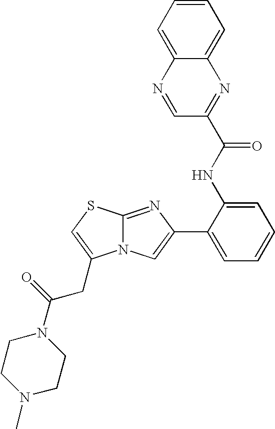 | | A′ | A |
| 650 | 570.2 | 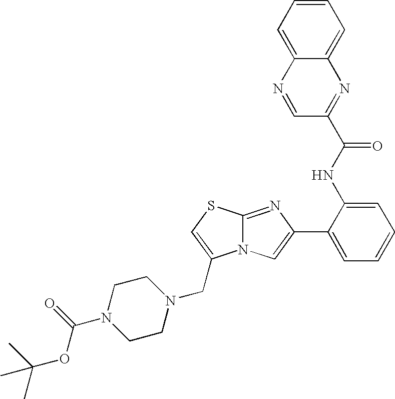 | | NA | |
| 651 | 512.2 | 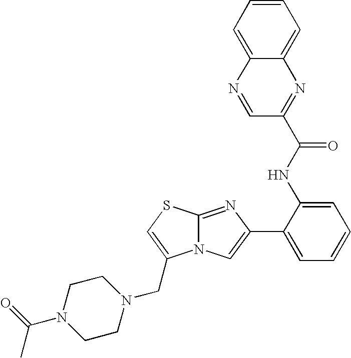 | | A′ | A |
| 655 | 393.0 | 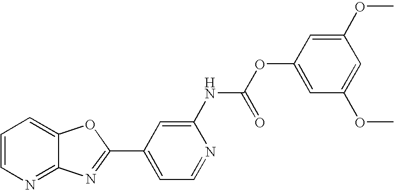 | | NA | C |
| 656 | 393.2 | 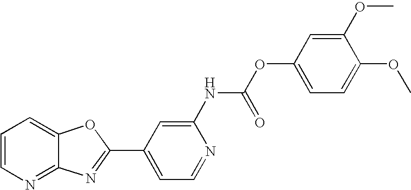 | | NA | C |
| 657 | 423.1 | 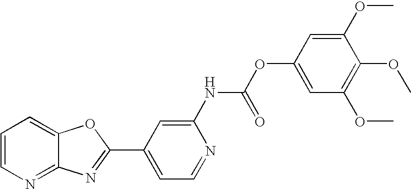 | | NA | C |
| 658 | 382.1 | 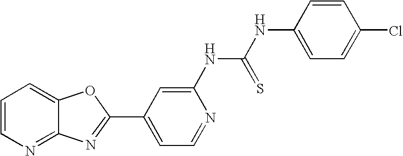 | | NA | C |
| 659 | 509.2 | 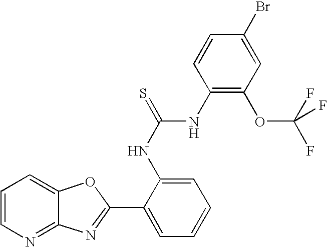 | | NA | C |
| 660 | 408.1 | 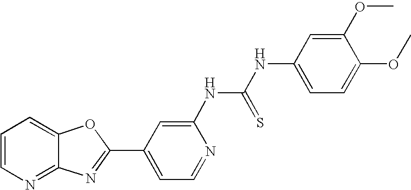 | | A | B |
| 661 | 408.2 | 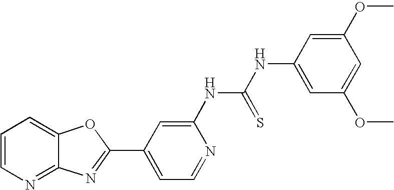 | | A′ | B |
| 662 | 378.2 | 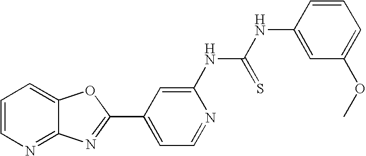 | | NA | C |
| 663 | 438.0 | 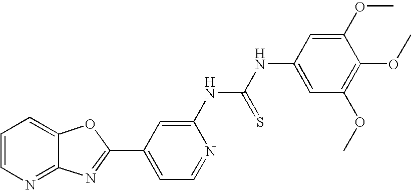 | | A | B |
| 664 | 477.8 | 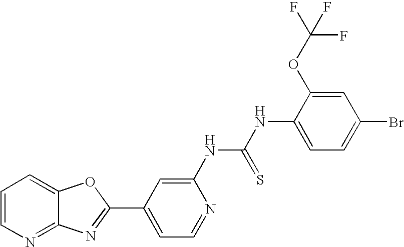 | | NA | C |
| 665 | 406.1 | 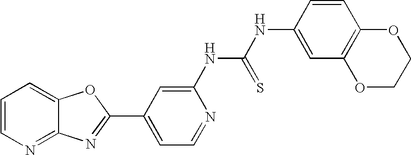 | | NA | C |
| 666 | 391.0 | 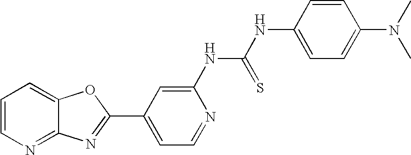 | | NA | C |
| 667 | 448.0 | 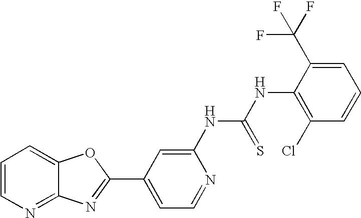 | | A | A |
| 668 | 410.0 | 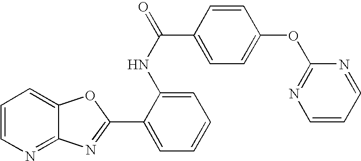 | | A | B |
| 669 | 392.0 | 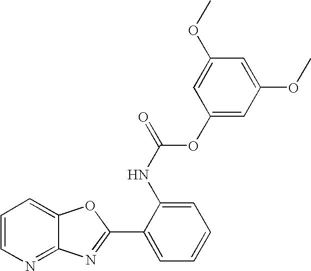 | | NA | C |
| 670 | 392.0 | 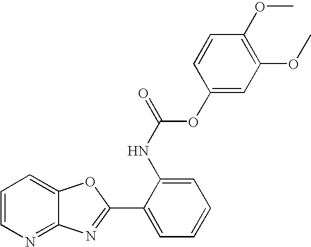 | | A | B |
| 671 | 422.0 | 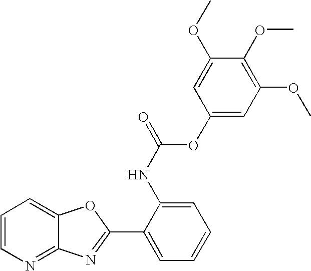 | | NA | C |
| 672 | 415.1 | 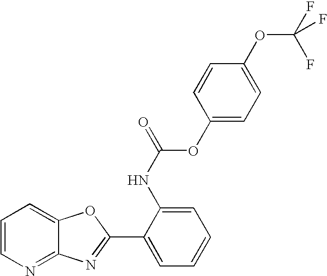 | | NA | C |
| 673 | | 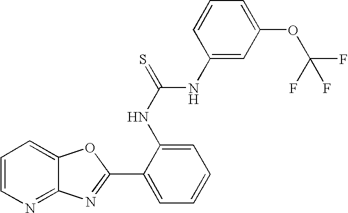 | | NA | C |
| 674 | | 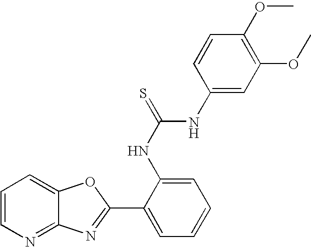 | | NA | C |
| 675 | 407.1 | 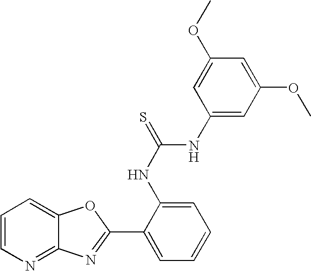 | | NA | C |
| 676 | | 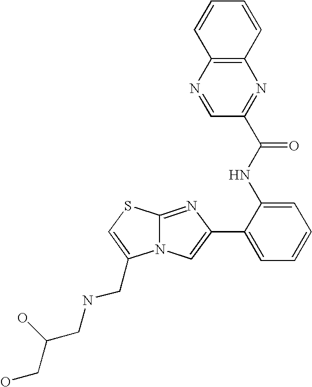 | | A′ | A |
| 677 | | 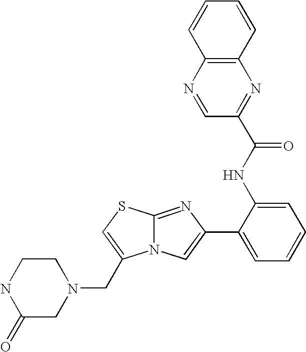 | A′ | A | |
| 678 | | 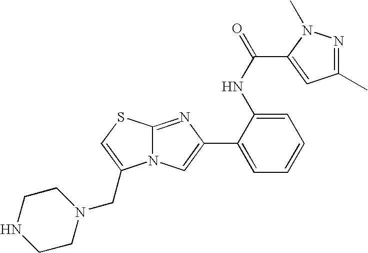 | | A | B |
| 679 | | 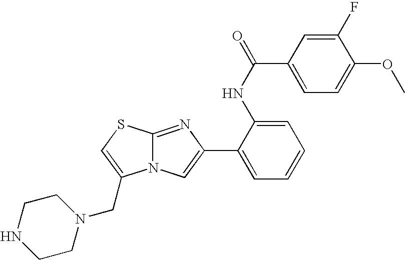 | | A | B |
| 680 | 484.2 | 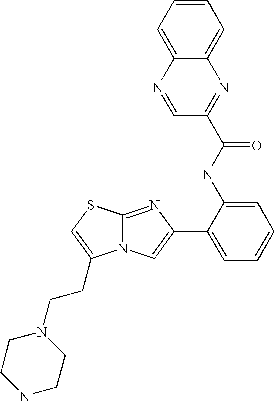 | | A′ | A |
| 681 | 522 | 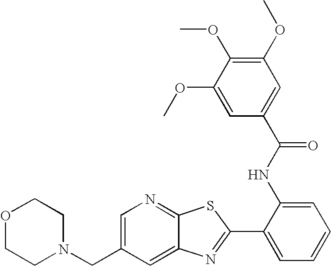 | | A | A |
| 682 | 492 | 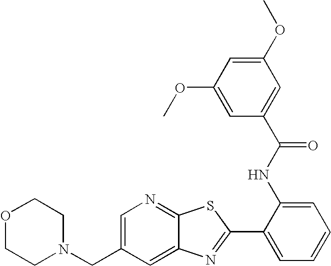 | | A′ | A |
| 683 | 475 | 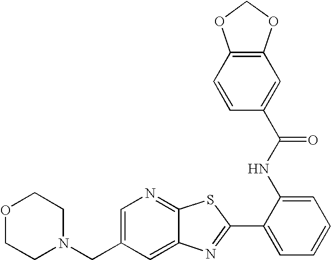 | | A | B |
| 684 | 460 | 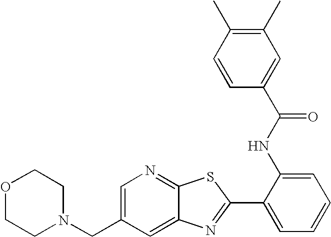 | | A | B |
| 685 | 456 | 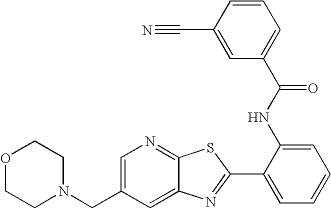 | | A′ | B |
| 686 | 475 | 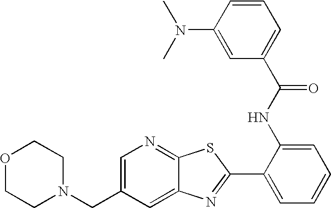 | | A′ | A |
| 687 | 467 | 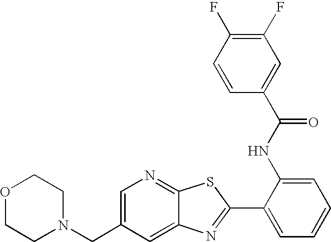 | | NA | C |
| 688 | 483 | 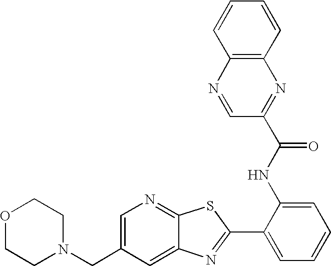 | | NA | C |
| 689 | 479 | 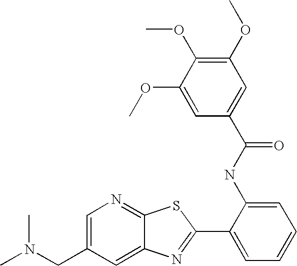 | | A | A |
| 690 | 483 | 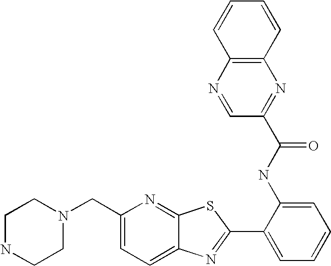 | | A′ | A |
| 692 | 469 | 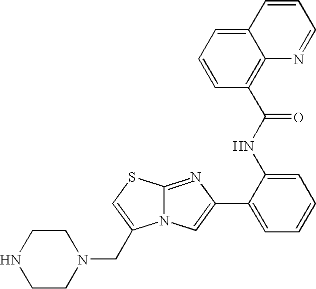 | | C | B |
| 695 | 454 | 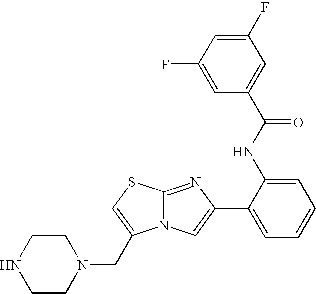 | | A′ | A |
| 697 | 596 | 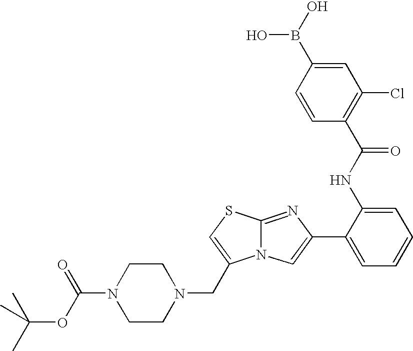 | | NA | C |
| 698 | 502 | 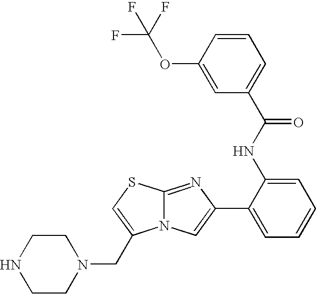 | | A′ | A |
| 699 | | 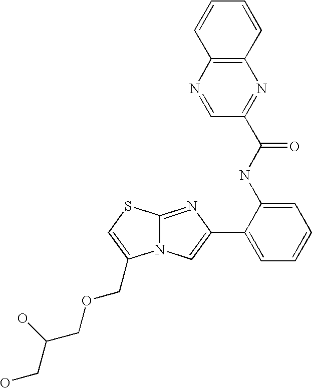 | | A | A |
| 700 | 512.2 | 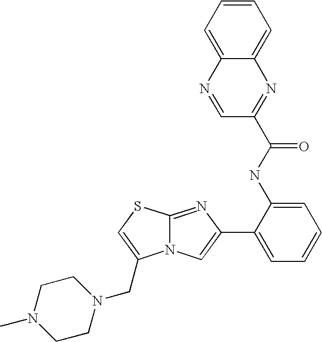 | | A′ | A |
| 701 | 477 | 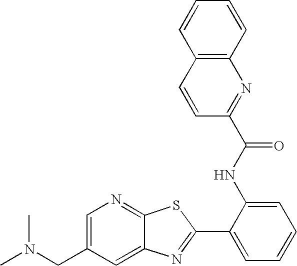 | | A′ | A |
| 702 | 534 | 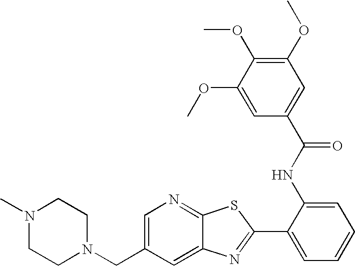 | | A | A |
| 703 | 468 | 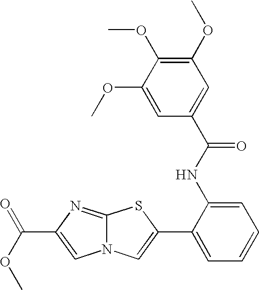 | | NA | C |
| 704 | 454 | 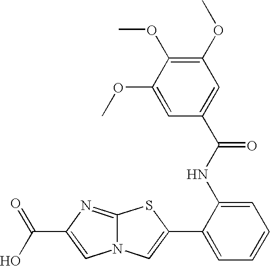 | | NA | C |
| 705 | 387 | 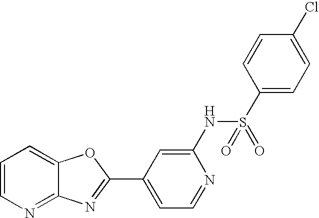 | | | |
| 706 | 445.1 | 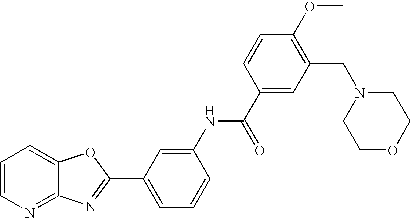 | | | |
| 707 | 386.1 | 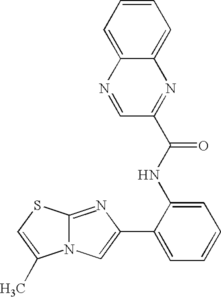 | | A′ | A |
| 708 | 494.2 | 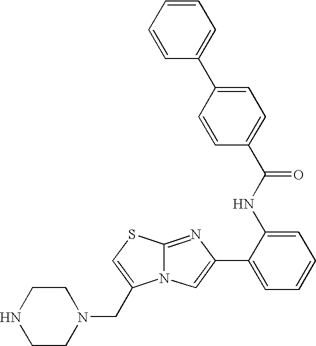 | | A′ | A |
| 709 | 494.1 | 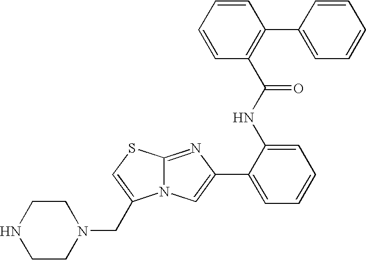 | | NA | C |
| 710 | 494.1 | 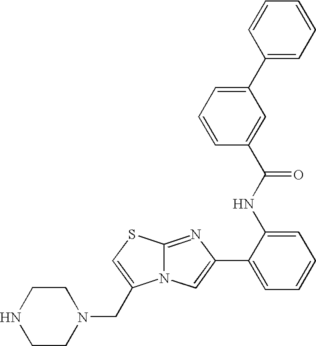 | | A′ | A |
| 711 | | 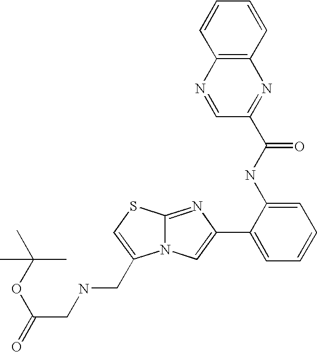 | | A | B |
| 714 | | 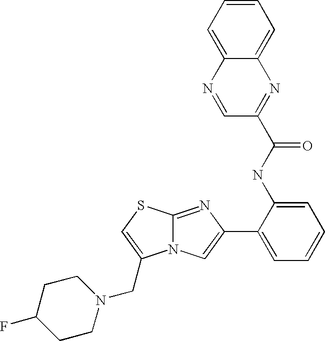 | | A | B |
| 715 | | 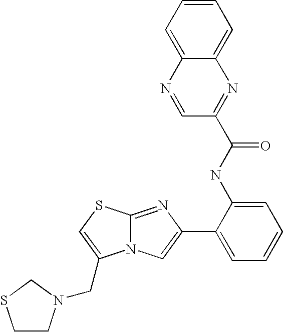 | | A′ | B |
| 716 | | 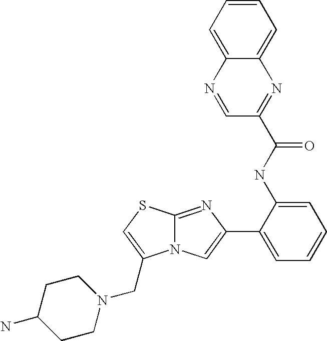 | | A′ | A |
| 717 | | 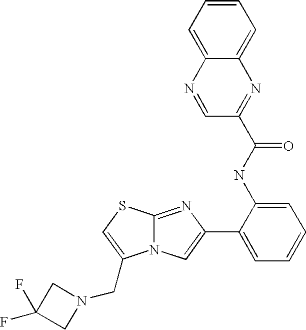 | | A | B |
| 718 | 444.1 | 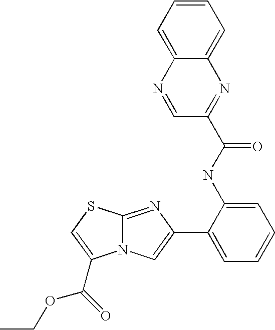 | | NA | C |
| 719 | 416 | 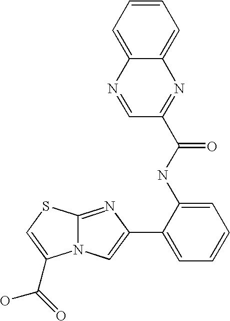 | | A | A |
| 720 | | 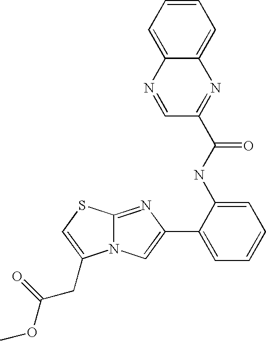 | | NA | C |
| 721 | | 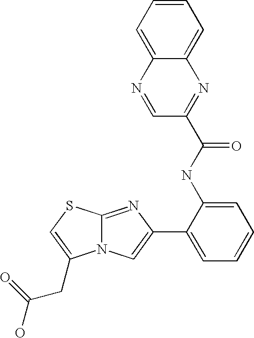 | | A | A |
| 722 | 449 | 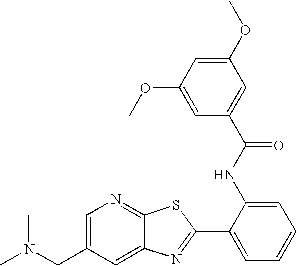 | | A′ | A |
| 723 | 490 | 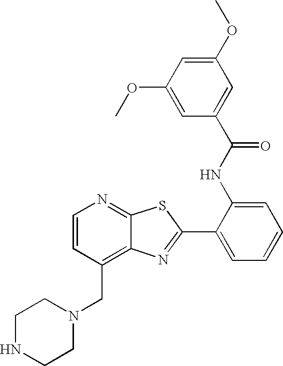 | | A | A |
| 724 | 482 | 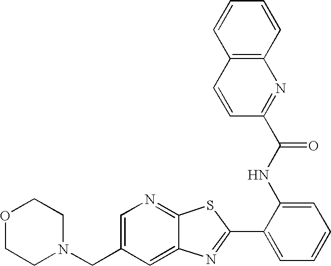 | | A′ | A |
| 725 | 505 | 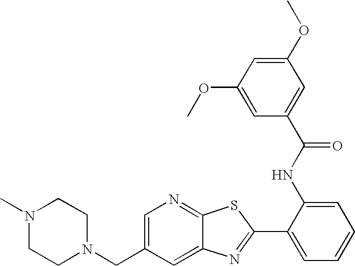 | | A′ | A |
| 726 | 491 | 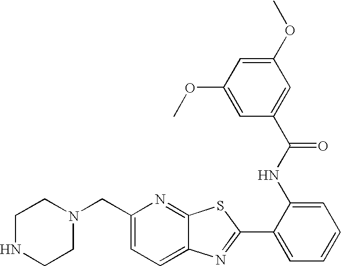 | | A′ | A |
| 727 | 458 | 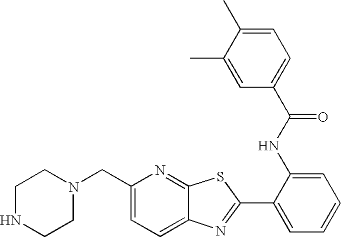 | | A′ | A |
| 728 | 466 | 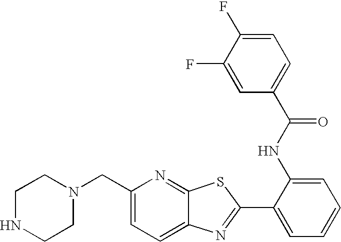 | | A | A |
| 729 | 481 | 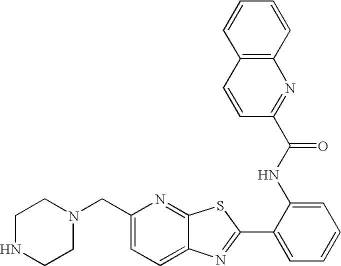 | | A′ | A |
| 730 | 488 | 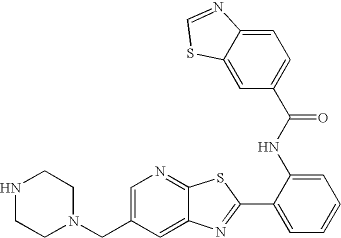 | | A | A |
| 731 | 498 | 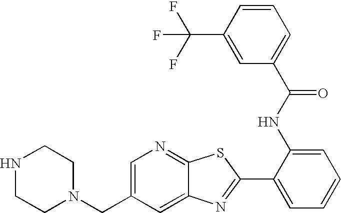 | | A | A |
| 732 | 497 | 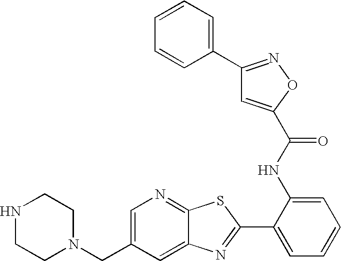 | | A′ | B |
| 733 | 484.2 | 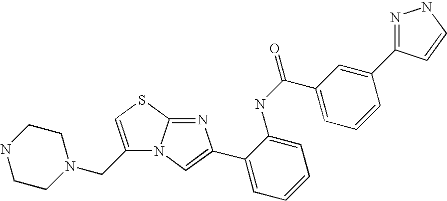 | | A′ | A |
| 735 | 514.2 | 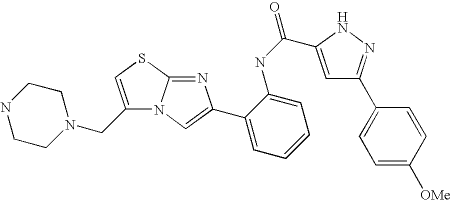 | | A′ | A |
| 736 | 514.2 | 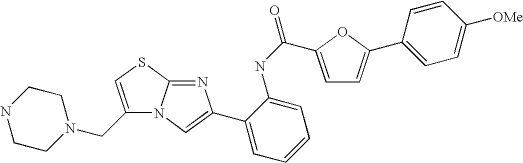 | | A′ | A |
| 737 | 500.1 | 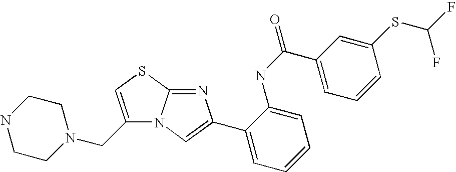 | | A′ | A |
| 738 | 448.1 | 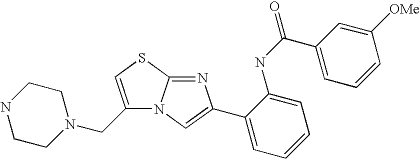 | | A | A |
| 739 | 424.1 | 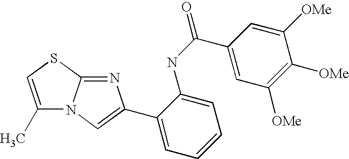 | | A′ | A |
| 740 | 377.1 | 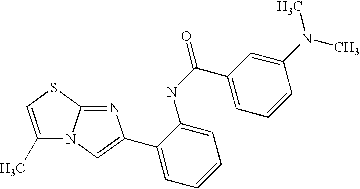 | | A′ | B |
| 741 | 498.1 | 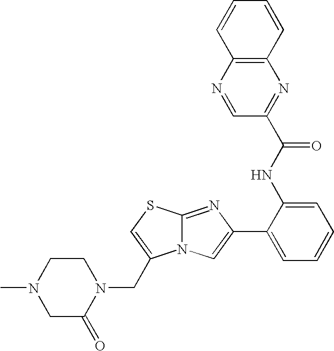 | | A′ | A |
| 742 | 487.1 | 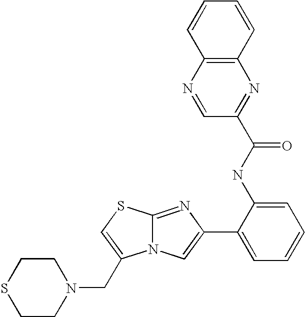 | | A′ | B |
| 743 | 466.1 | 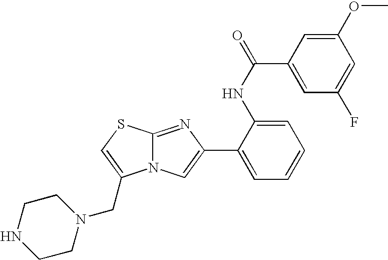 | | A | A |
| 744 | 437.1 | 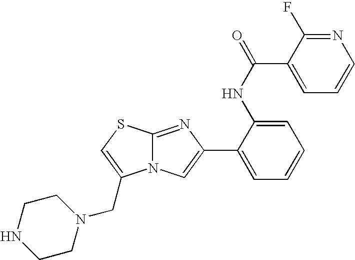 | | B | A |
| 745 | 452.1 | 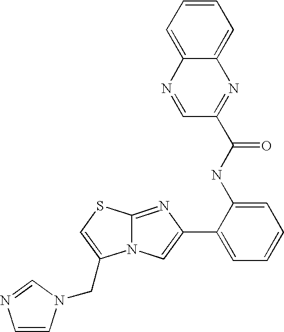 | | A′ | A |

[symbol]

indicates data missing or illegible when filed

TABLE 5

| COM- | | | | |
|---|---|---|---|---|
| POUND | | | IC₅₀ FP | IC₅₀ MS |
| NO | [M + H]+ | STRUCTURE | ASSAY | ASSAY |
| 13 | | 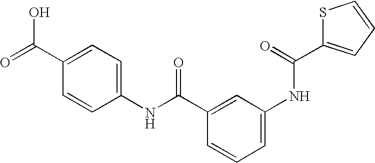 | A | |
| 14 | | 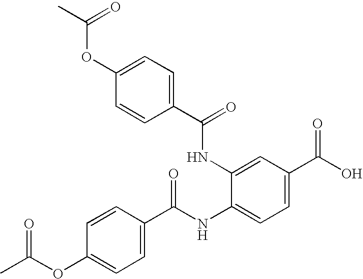 | B | |
| 15 | | 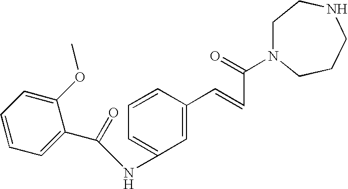 | C | |
| 23 | 359.4 | 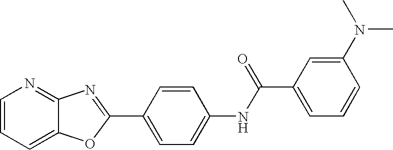 | D | |
| 25 | 341.3 | 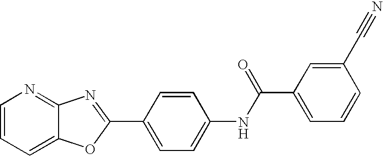 | B | |
| 26 | 341.4 | 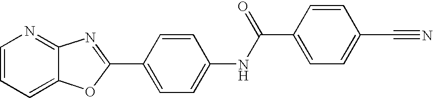 | B | |
| 28 | 401.1 | 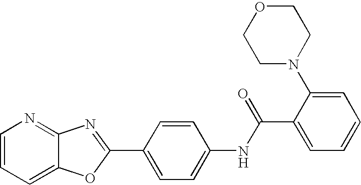 | D | |
| 30 | 380.0 | 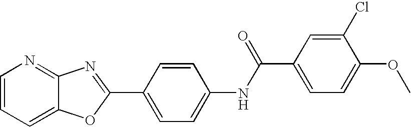 | D | |
| 44 | 341.0 | 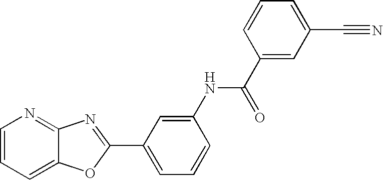 | D | |

TABLE 6

| COMPOUND NO | STRUCTURE | ED₅₀ |
|---|---|---|
| 11 | 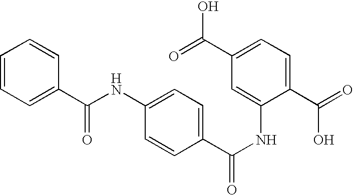 | N/A |
| 12 | 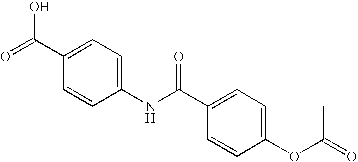 | N/A |

TABLE 8

| COMPOUND | | ED₅₀ ATP | IC₅₀ ATP |
|---|---|---|---|
| NO | STRUCTURE | ASSAY | ASSAY |
| 52 | 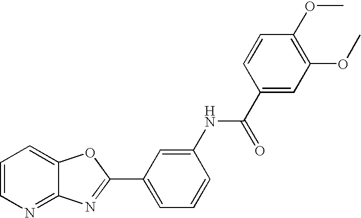 | A | |
| 42 | 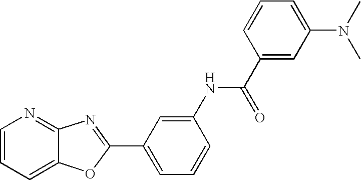 | A | |
| 49 | 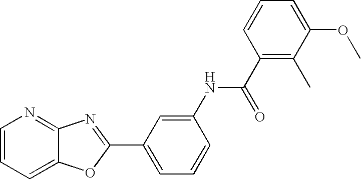 | A | |
| 115 | 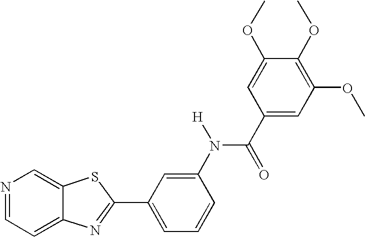 | D | |
| 79 | 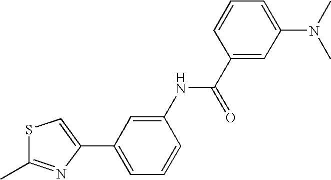 | A | |
| 117 | 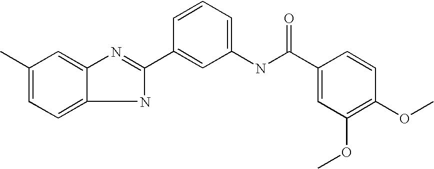 | | B |
| 120 | 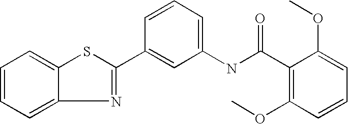 | A | |
| 121 | 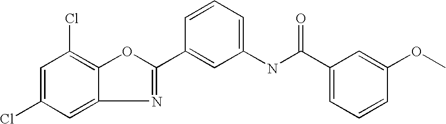 | NA | |
| 123 | 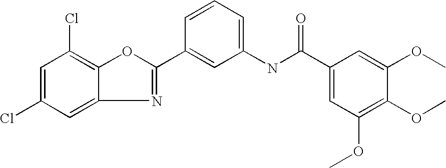 | D | |
| 85 | 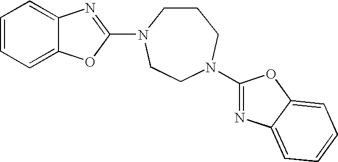 | NA | |
| 86 | 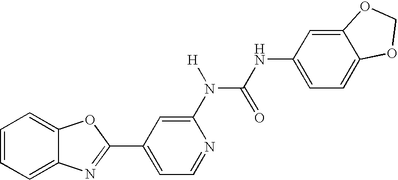 | A | |
| 87 | 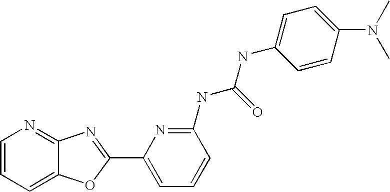 | NA | |
| 88 | 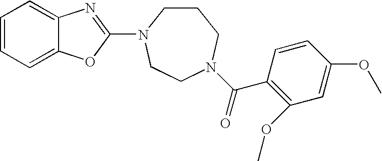 | NA | |
| 89 | 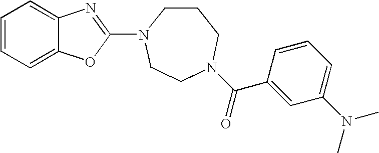 | | A |
| 90 | 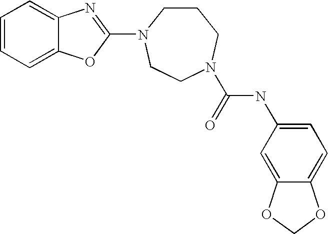 | D | |
| 91 | 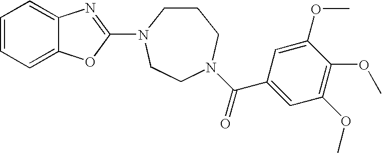 | | A |
| 92 | 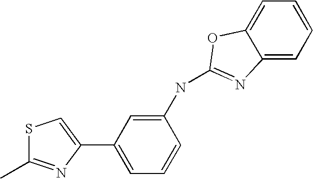 | | B |
| 93 | 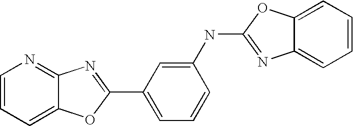 | D | |
| 94 | 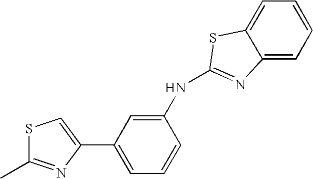 | D | |
| 95 | 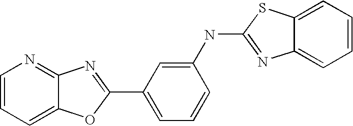 | NA | |
| 97 | 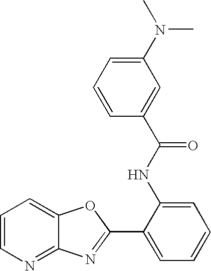 | A | |
| 98 | 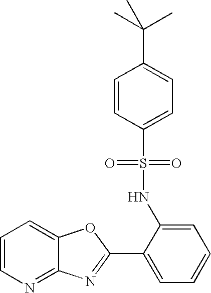 | NA | |
| 99 | 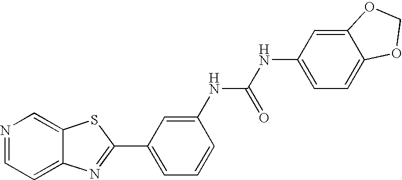 | A | |
| 100 | 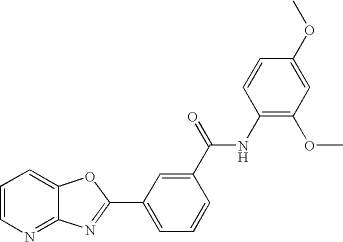 | A | |
| 101 | 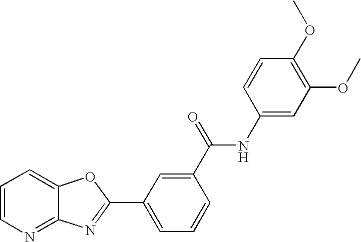 | C | |
| 102 | 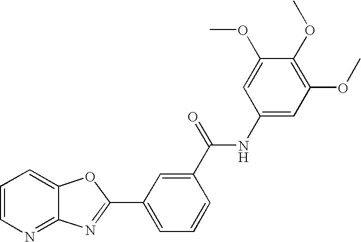 | A | |
| 103 | 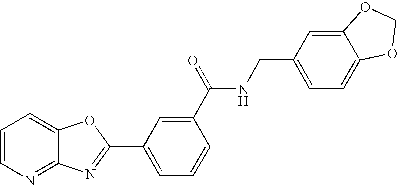 | NA | |
| 104 | 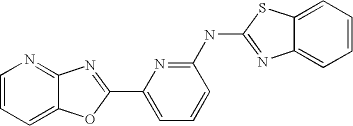 | A | |
| 105 | 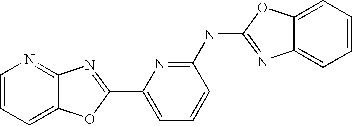 | A | |
| 133 | 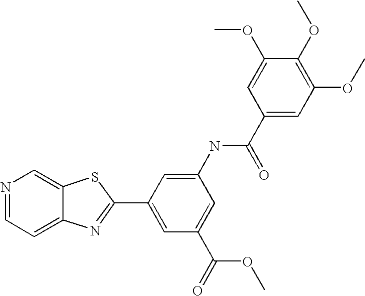 | NA | |
| 134 | 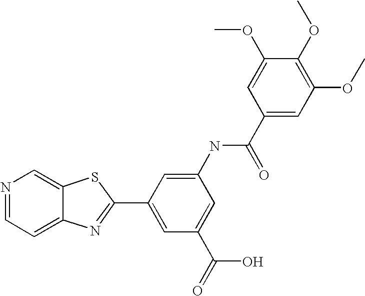 | NA | |

In one embodiment [symbol] -modulating compounds of the invention are represented by Structural Formula (I):

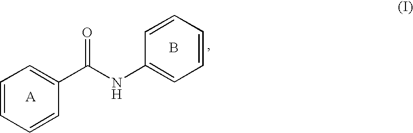

or a salt thereof where:

Ring A is optionally substituted; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In certain embodiments, Ring B is substituted with at least a carboxy group.

In certain embodiments, Ring B is substituted with at least a substituted or unsubstituted arylcarboxamine, a substituted or unsubstituted aralkylcarboxamine or a polycyclic aryl group.

In certain embodiments, Ring B is substituted with at least a substituted or unsubstituted heteroaryl group or a substituted or unsubstituted heterocyclylcarbonylethenyl group.

In another embodiment, [symbol] -modulating compounds of the invention are represented by Structural Formula (II):

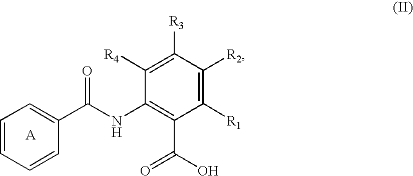

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆—NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In a further embodiment, [symbol] -modulating compounds of the invention are represented by Structural Formula (IIa):

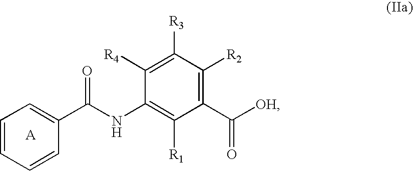

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another embodiment, [symbol] -modulating compounds of the invention are represented by Structural Formula (II):

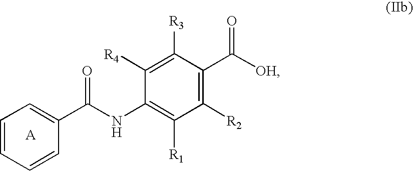

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ in Structural Formulas (II)-(IIb) are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention provides novel [symbol] -modulating compounds of Formula (III):

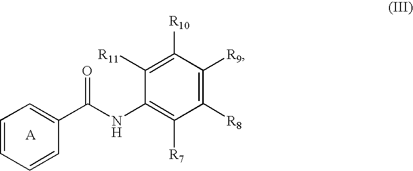

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆—OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In certain such embodiments, R₈ is selected from thiazolopyridyl, imidazothiazolyl, benzoxazinonyl, or imidazopyridyl.

Particular examples of R₈, where

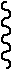

indicates attachment to the remainder of Structural Formula (III), include:

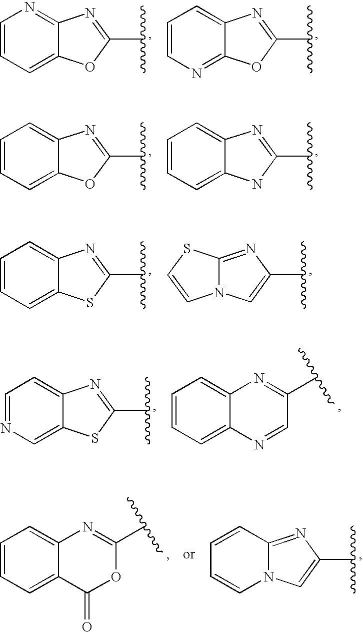

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

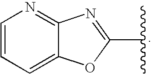

In certain embodiments, R₈ is

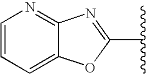

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

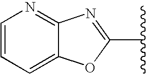

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

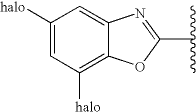

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

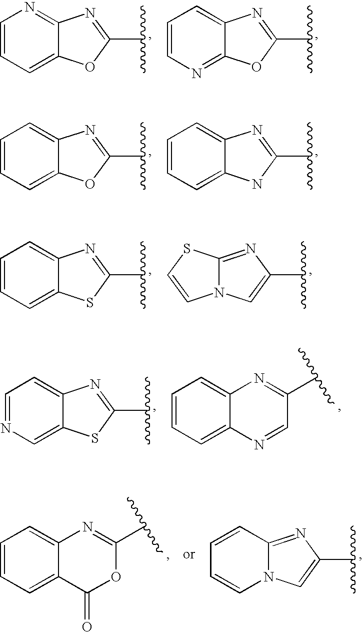

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)—N(CH₃)₂, or —C(O)-piperazinyl. In certain such embodiments, when R₈ is

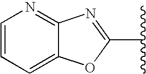

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

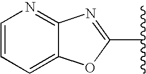

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁, is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In yet another aspect, the invention provides novel [symbol] -modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′R₂′; OC(O)R₂′; S(O)₂R₂′; S(O)₂NR₂′R₂′; NR₂′C(O)NR₂′R₂′; NR₂′C(O)C(O)C(O)R₂′; NR₂′ C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇, NR₇′R₇′, NR₇′R₈′, NR₈′R₈′, COOR₁′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₇′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)Rod, C(O)NR₁₀′R₁₀, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R; C1-C10 alkyl substituted with 1-3 independent R₉′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₁′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the invention, each Ar, L, and Ar′ is independently an optionally substituted 5- to 7-membered monocyclic ring system or an optionally substituted 9- to 12-membered bicyclic ring system.

According to another preferred embodiment,
Ar is

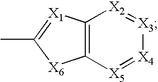

X₁, X₂, X₃, X₄, and Xs are independently selected from CR₁′ and N; and
X₆ is selected from NR₁′, O, and S;

According to yet another preferred embodiment, X₁ and X₂ are N; X₃, X₄, and Xs are CR₁′; and X₆ is O.

According to still yet another preferred embodiment, X₁ and X₃ are N; X₂, X₄, and X₅ are CR₁′; and X₆ is O.

According to still yet another preferred embodiment, X₁ and X₄ are N; X₂, X₃, and X₅ are CR₁′; and X₆ is O.

According to still yet another preferred embodiment, X₁ and X₅ are N; X₂, X₃, and X₄ are CR₁′; and X₆ is O.

In another embodiment, the compounds of the formula above are those wherein J is NR₁′, K is absent, and M is C(O).

In yet another embodiment, the compounds of the formula above are those wherein J is absent, K is NR₁′, and M is C(O).

In a further embodiment, compounds of formula (IV) are those where when J is absent and K is NR₁′, M is not C(O) and when J is NR₁′ and K is absent, M is not C(O).

In a preferred embodiment, the compounds above are those wherein L is an optionally substituted 5- to 7-membered carbocyclic or heterocyclic aryl group.

In yet another preferred embodiment, the compounds are those wherein L is an optionally substituted phenylene, pyridinylene, imidazolylene, oxazolylene, or thiazolylene.

In a particularly preferred embodiment, L is an optionally substituted phenylene.

In another particularly preferred embodiment, L is an optionally substituted pyridinylene.

In an even more preferred embodiment, L is phenylene.

In another even more preferred embodiment, L is pyridinylene.

In either of these embodiments, Ar and J may be attached to L at the ortho-, meta-, or para-positions. Particularly preferred are those embodiments where attachment is at the meta-position.

In certain embodiments, L is not phenylene when Ar′ is phenyl. Examples of such embodiments include embodiments where L is an optionally substituted heterocyclic aryl group and Ar′ is an optionally substituted carbocyclic or heterocyclic aryl group, or wherein L is an optionally substituted carbocyclic or heterocyclic aryl group and Ar′ is an optionally substituted heterocyclic aryl group.

In yet another aspect, the invention provides novel [symbol] -modulating compounds of Formula (I) or a salt thereof, wherein

Ring A is substituted with at least one R₁′ group;

R₁′, R₂′, R₃′, R₄′, R₅′, R₆′, R₇′, R₈′, R₉′, R₁₀′, and R₁₁′ are as defined above; each haloalkyl is independently a C1-C10 alkyl substituted with one or more halogen atoms, selected from F, Cl, Br, or I, wherein the number of halogen atoms may not exceed that number that results in a perhaloalkyl group;

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′; and

Ring B is substituted with at least one

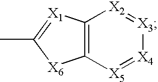

wherein

X₁, X₂, X₃, X₄, and X₅ are independently selected from CR₁′ and N; and

X₆ is selected from NR₁′, O, and S.

In a preferred embodiment, Ring B is phenyl or pyridinyl.

In a further aspect, the invention provides novel [symbol] modulating compounds of Formula (IVa):

Het-L-Q-Ar′ (IVa)

or a salt thereof, wherein:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′, —NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′—,

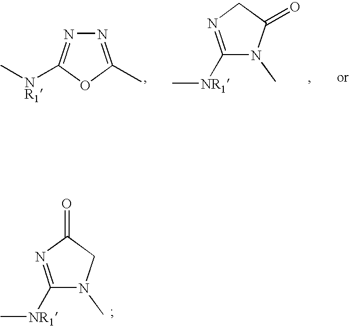

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In certain embodiments, when Het is a polycyclic heteroaryl, L is optionally substituted phenylene, and Ar′ is optionally substituted phenyl; then Q is not —NH—C(O)—.

In certain embodiments, Het and Q are attached to L in a 1-, 2- or 1-,3-configuration (e.g., when L is phenylene, Het and Q are attached in an ortho or a meta orientation). In certain embodiments where Het and Q are attached to L in a 1-,3-configuration, if Het is benzoxazolyl, L is pyridylene and Q is —NH—C(O)—NH, then Ar′ is not 3,4 dioxymethlyene phenyl; if Het is methyl thiazolyl, L is phenylene and Q is —NH—C(O)—, then Ar′ is not 3-dimethylamino phenyl; if Het is oxazolopyridyl, L is pyridylene and Q is —NH—C(O)—NH, then Ar′ is not 4-dimethylamino phenyl; if Het is oxazolopyridyl or benzoxazolyl and L is

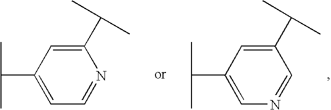

then Q is not —NH—(SO)₂—; and if Het is oxazolopyridyl, L is

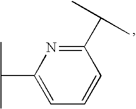

and Q is —NH—C(O)—, then Ar′ is not 3,4 dimethoxyphenyl or pyridyl.

When Het is substituted, it is typically substituted at up to 2 carbon atoms with a substituent independently selected from R₁₂, N(R₁₂)₂, NH(R₁₂), OR₁₂; C(O)—NH—R₁₂, C(O)—N(R₁₂)₂, N(R₁₂)—OR₁₂, CH—N(R₁₂)₂, C(O)OR₁₂, C(O)OH,

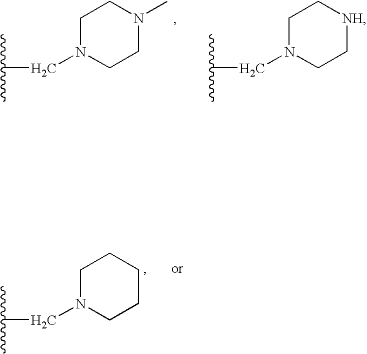

or

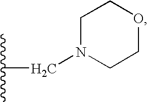

where each R₁₂ is independently selected from optionally substituted C₁-C₃ straight or branched alkyl.

In certain embodiments, Het is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In other embodiments, Het comprises one ring N heteroatom and 1 to 2 additional ring heteroatoms independently selected from N, O or S, such as thiazolyl, triazolyl, oxadiazolyl, thiazolopyridyl, imidazothiazolyl, benzoxazinonyl, or imidazopyridyl.

Particular examples of Het include:

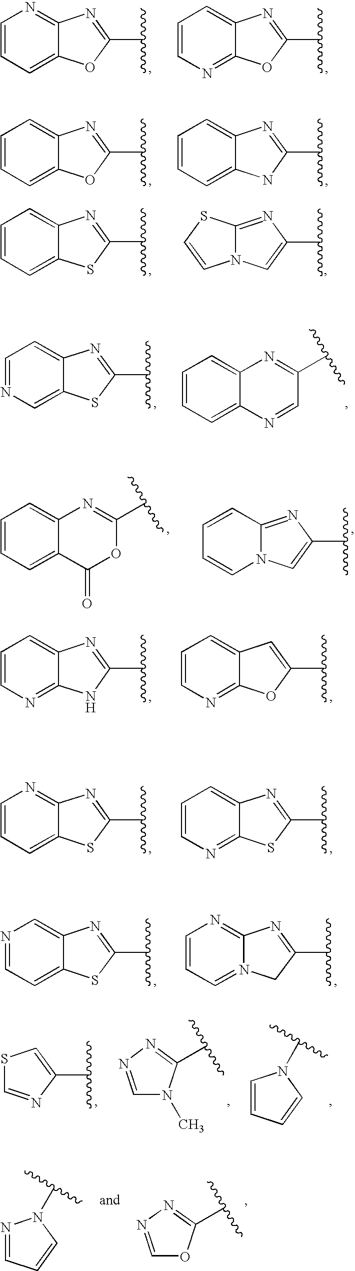

where up to 2 ring carbons not immediately adjacent to the indicated attachment point are independently substituted with optionally substituted C₁-C₃ straight or branched alkyl, phenyl, halo, N(R₁₂)₂, NH(R₁₂), OR₁₂, C(O)—NH—R₁₂, C(O)—N(R₁₂)₂, N(R₁₂)—OR₁₂, CH₁₂—N(R₁₂)₂, C(O)OR₁₂, C(O)OH,

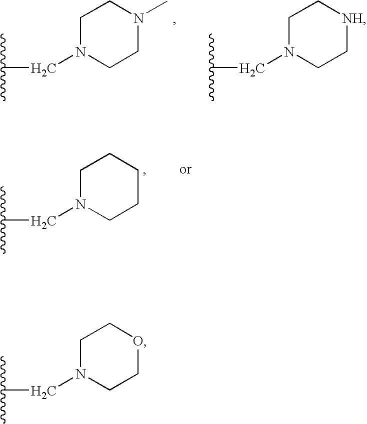

wherein each R₁₂ is independently selected from optionally substituted C₁-C₃ straight or branched alkyl.

In certain embodiments, L is selected from

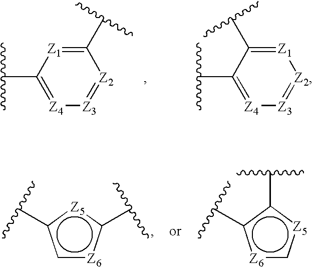

wherein:

each of Z₁, Z₂, Z₃ and is independently selected from CH or N, wherein not more than three of said Z₁, Z₂, Z₃ or Z₄ is N;

each of Z₅ and Z₆ is independently selected from C, N, O or S, provided that at least one of Z and Z₆ is N; and

L is optionally substituted at 1 to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

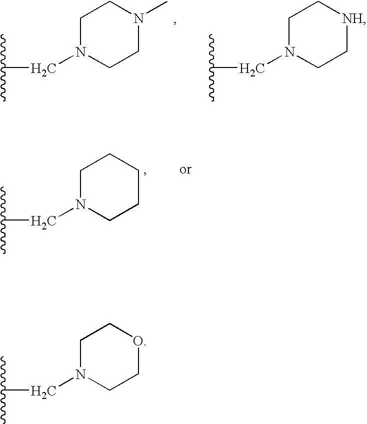

In preferred embodiments, L is selected from phenylene or pyridylene, such as unsubstituted phenylene or phenylene substituted with a single substituent selected from C(O)OCH₃, C(O)OH, CH₂OH, N(CH₃)₂, or CH₂N(CH₃)₂, or unsubstituted pyridylene.

In certain embodiments, Q is selected from —NH—C(O)—, —NH—S(O)₂—, —NH—C(O)—NH—, —C(O)—NH—, —CH₂—, —N(CH₃)—C(O)—NH—, —NH—C(O)—N(CH₃)—, or —NH—S(O)₂—NH—, particularly —NH—C(O)—, —C(O)—NH—, —NH—, —NH—C(O)—NH, or —NH—S(O)₂—.

In certain embodiments, Ar′ is selected from optionally substituted phenyl, benzothiazolyl, or benzoxazolyl. When Ar′ is phenyl, typical optional substituents are 1 to 3 substituents independently selected from halo, (optionally substituted C₁-C₃ straight or branched alkyl), O-(optionally substituted C₁-C₃ straight or branched alkyl), S-(optionally substituted C₁-C₃ straight or branched alkyl), N(CH₃)₂ or optionally substituted heterocyclyl, or wherein two substituents on adjacent ring atoms are taken together to form a dioxymethylene.

In certain embodiments, Het is selected from

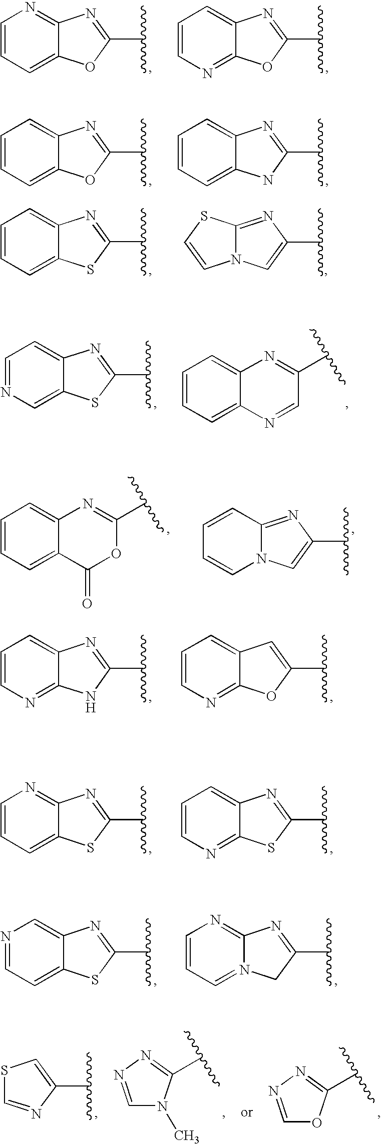

and wherein up to 2 ring carbons not immediately adjacent to the indicated attachment point are independently substituted with optionally substituted C₁-C₃ straight or branched alkyl, phenyl or halo;

L is selected from unsubstituted phenylene, phenylene substituted with a single substituent selected from C(O)OCH₃, C(O)OH, CH₂OH, N(CH₃)₂, or CH₂N(CH₃)₂, or unsubstituted pyridylene;

Q is selected from —NH—C(O)—, —C(O)—NH—, —NH—, —NH—C(O)—NH, or —NH—S(O)₂—; and

Ar′ is selected from optionally substituted phenyl, benzothiazolyl, or benzoxazolyl, wherein said phenyl is optionally substituted with 1 to 3 substituents independently selected from chloro, methyl, O-methyl, S-methyl, N(CH₃)₂, morpholino, or 3,4 dioxymethylene.

In certain embodiments, Q is selected from —NH—C(O)—, —C(O)—NH—, —NH— or —NH—C(O)—NH.

In certain embodiments, the substituents on Ar′ are selected from chloro, methyl, O-methyl, S-methyl or N(CH₃)₂. In certain embodiments, the only substituent on Ar′ is an O-methyl group, particularly an O-methyl group ortho or meta to Q. In certain embodiments, when there are two or more O-methyl-groups or Ar′, at least one is ortho or meta to Q.

In certain embodiments, L is pyridyl and Het and Q are at the 1,3- or 2,4-position with respect to the pyridyl nitrogen atom. In certain such embodiments, Q is —NH—S(O)₂—.

In certain embodiments where L is further substituted, the substituent is typically meta to both Het and Q.

In certain embodiments, Q is —NH— and Het is thiazolyl or oxazolopyridyl.

In certain embodiments, Q is —NH— and Ar is benzothiazolyl or benzoxazolyl.

In certain embodiments, such as when the sirtuin modulator is a sirtuin activator, L is

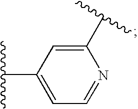

and Q is —NH—(SO)₂—. In certain such embodiments, Het is oxazolopyridyl. When L, Q and optionally Het have these values, Ar′ is advantageously naphthyl or phenyl, where Ar′ is optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments, L is

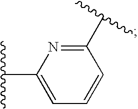

and Q is —NH—C(O)—. In certain such embodiments, Het is oxazolopyridyl. When L, Q and optionally Het have these values, Ar′ is advantageously pyridyl or phenyl optionally substituted with 1 to 3 substituents independently selected from CN, halo, (C1-C3 straight or branched alkyl), O—(C1-C3 straight or branched alkyl), N(C1-C3 straight or branched alkyl)₂, or a 5 to 6-membered heterocycle.

In certain embodiments,

Het comprises one N heteroatom and 1 to 2 additional heteroatoms independently selected from N, O or S;

L is

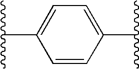

and is optionally substituted;

Q is —NH—C(O)—; and

Ar′ is phenyl substituted with 1 to 3 substituents independently selected from CN, halo, C₁-C₃ straight or branched alkyl, O—(C₁-C₃ straight or branched alkyl), N(C₁-C₃ straight or branched alkyl)₂, or a 5 to 6-membered heterocycle,

wherein when R₈ is unsubstituted

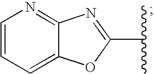

then ring A is:
a) not simultaneously substituted at the 2- and 6-positions with O—(C₁-C₃ straight or branched alkyl);
b) not simultaneously substituted at the 2-position with C₁-C₃ straight or branched alkyl or O—(C₁-C₃ straight or branched alkyl) and at the 3-position with O—(C₁-C₃ straight or branched alkyl);
c) not substituted at the 4-position with O—(C₁-C₃ straight or branched alkyl) unless simultaneously substituted at the 3-position with halo or O—(C₁-C₃ straight or branched alkyl) and unsubstituted at all other positions;
   not substituted at the 4-position with N(C₁-C₃ straight or branched alkyl)₂, or said 5 to 6-membered heterocycle. In certain such embodiments, L is unsubstituted and/or Het is oxazolopyridyl.

In yet another aspect, the invention provides novel [symbol] -modulating compounds of Formula (V):

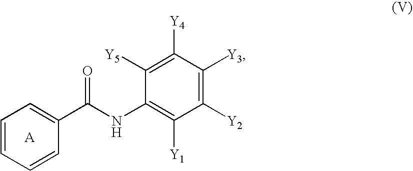

or a salt thereof, wherein:

Ring A is optionally substituted with at least one R₁′ group;

Y₁, Y₂, Y₃, Y₄, and Y₅ are independently R₁′;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R)S(O)₂R₉′; S(O)₂NR₉′R₉′; OC(O)R₉′; NR₉′C(O)NR₉′R₉′; NR₉′C(O)C(O)R₉′; N₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆, halo, CF₃, OR₉′, SR₉′, NR₉′R₉′, COOR₉′, NO₂, CN, C(O)R₉′, C(O)NR₉′R₉′, NH—IC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the above compound,

either Y₂ or Y₃ is

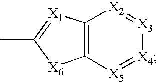

X₁, X₂, X₃, X₄, and X₅ are independently selected from CR₁′ and N; and

X₆ is selected from NR₁′, O, and S.

According to an even more preferred embodiment, X₁ and X₂ are N; X₃, X₄, and Xs are CR₁′; and X₆ is O.

According to another even more preferred embodiment, X₁ and X₃ are N; X₂, X₄, and X₅ are CR₁′; and X₆ is O.

According to another even more preferred embodiment, X₁ and X₄ are N; X₂, X₃, and X₅ are CR₁′; and X₆ is O.

According to another even more preferred embodiment, X₁ and X₅ are N; X₂, X₃, and X₄ are CR₁′; and X₆ is O.

In another aspect, the invention provides [symbol] -modulating compounds of Structural Formula (VII):

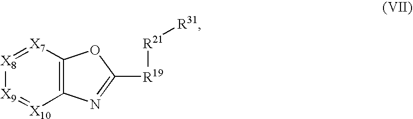

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

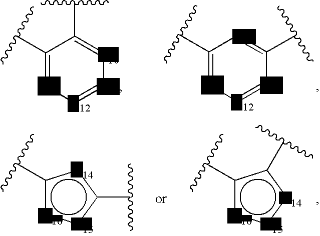

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁═CR₁′—, —NR₁′—S(O)—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(—═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

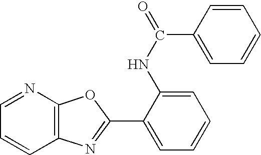

or that when R¹⁹ is

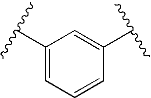

and R² is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

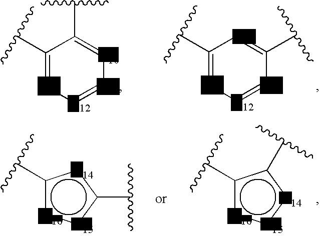

wherein
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, S—NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not

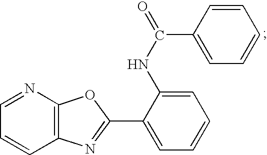

and

when X₈ and X₉ are each independently selected from CR²⁰ or CR₁′, R¹⁹ is

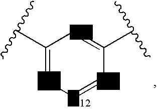

and each of Z₁₀, Z₁₁, Z₁₂ and Z₁₃ is independently selected from CR²⁰, or CR₁′, then:

a) at least one of X₈ and X₉ is not CH; or

b) at least one of Z₁₀, Z₁₁, Z₁₂ and Z₁₃ is CR²⁰, wherein R²⁰ is a solubilizing group.

In certain embodiments, when Z₁₂ is CR²⁰ and R²⁰ is a solubilizing group, the solubilizing group is not —C(O)OCH₂CH₃, —COOH,

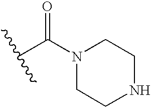

or

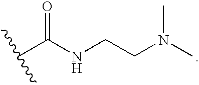

In certain embodiments, when X₈ and X₉ are each independently selected from CR²⁰ or CR₁′, R¹⁹ is

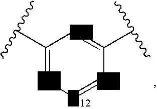

and each of Z₁₀, Z₁₁, Z₁₂ and Z₁₃ is independently selected from CR²⁰, or CR₁′, then:
a) at least one of Xs and X₉ is not CH; or
b) at least one of Z₁₀, Z₁₁ and Z₁₃ is CR²⁰, wherein R²⁰ is a solubilizing group.

In certain embodiments, when R¹⁹ is

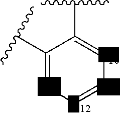

and each of Z₁₀, Z₁₁, Z₁₂ and Z₁₃ is CR²⁰, or CR₁′; X₈ and X₉ are CR²⁰ or CR₁′; R²¹ is —NHC(O)—; and R³¹ is optionally substituted phenyl, then R³¹ is a substituted phenyl, at least one R₁′ in a CR₁′ moiety is optionally substituted C₁-C₃ straight or branched alkyl, or at least one R²⁰ in a CR²⁰ is a solubilizing group, or a combination thereof.

In certain embodiments, R¹⁹ is selected from phenyl, pyridyl, thienyl or furyl.

In certain embodiments, R¹⁹ is

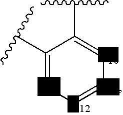

wherein each of Z₁₀, Z₁₁, Z₁₂ and Z₁₃ is independently selected from CR²⁰ or CR₁′; and

R²¹ is —NH—C(O)—; and

R³¹ is a substituted phenyl.

In certain such embodiments, when X₉ is N, R³¹ is not 2,4 dimethoxyphenyl and/or when X₁₀ is N, R³¹ is not halo substituted phenyl; 3,4-dioxoethylenephenyl; or 3,5-dimethoxyphenyl.

In preferred embodiments, R³¹ is optionally substituted with 1 to 3 substituents independently selected from —OCH₃, —CH₃, —N(CH₃)₂, pyrazinoxy or a solubilizing group. Suitable examples of R³¹ include 3-methoxy-4-((4-methylpiperazin-1-yl)methyl)phenyl, 3-methoxy-4-morpholinomethylphenyl, 3-methoxy-4-diaminomethylphenyl, 3-methoxy-4-((pyrrolidin-1-yl)methyl)phenyl, 3,4-dimethoxyphenyl, 3,5-dimethoxyphenyl, 2,3,4-trimethoxyphenyl, 3,4,5-trimethoxyphenyl, 2-dimethylaminophenyl, 3-dimethylaminophenyl, 4-dimethylaminophenyl, or 3,5-dimethylphenyl.

In certain embodiments, R¹⁹ is selected from

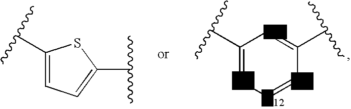

wherein one of Z₁₀, Z₁₁, Z₁₂, and Z₁₃ is N and the others are independently selected from CR²⁰ or CR₁′;

R²¹ is selected from —NH—, —NH—C(O)—, —NH—C(O)—NH, —NH—C(S)—NH— or —NH—S(O)₂—; and

R³¹ is selected from an optionally substituted phenyl, an optionally substituted naphthyl, or an optionally substituted heteroaryl.

In certain such embodiments,
a) when R²¹ is —NH—S(O)₂—, either:
   i) Z₁₀ is N; or
   ii) Z₁₁ is N and R³¹ is halophenyl or 2-methoxy-5-methylphenyl;
b) when R¹⁹ is

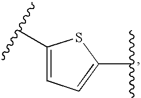

R³¹ is not 4-dimethylaminophenyl, 2,3,4-trimethoxyphenyl, or 3,5 dimethoxyphenyl; and/or
c) when R²¹ is —NH—C(O)—NH— and Z₁₀ is N, R³¹ is not 4-dimethylaminophenyl.

In certain such embodiments, R³¹ is selected from optionally substituted phenyl, benzothiazolyl, or benzoxazolyl.

In yet another embodiment, the invention provides [symbol] -modulating compounds of Structural Formula (VIII):

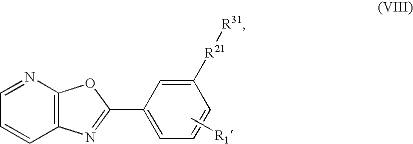

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that

when R₁′ is methyl, and R²¹ is —NH—C(O)—, R³¹ is not

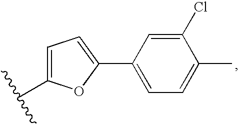

1-methoxynaphthyl, 2-methoxynaphthyl, or unsubstituted 2-thienyl;

when R₁′ is methyl, and R²¹ is —NH—C(O)—CH═CH—, R³¹ is not

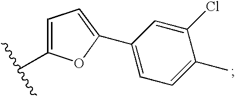

when R₁′ is methyl, and R²¹ is —NH—C(O)—CH—O—, R³¹ is not unsubstituted naphthyl, 2-methoxy, 4-nitrophenyl, 4-chloro-2-methylphenyl, or 4-t-butylphenyl; and

when R²¹ is —NH—C(O)—, R³¹ is not optionally substituted phenyl.

In certain embodiments, R²¹ is —NH—C(O)—; and R³¹ is phenyl optionally substituted with 1 to 3 substituents independently selected from —OCH₃, —CH₃, —N(CH₃)₂, or a solubilizing group.

In certain such embodiments, R²¹ is —NH—C(O)— and R³¹ is selected from unsubstituted phenyl, 2-methoxyphenyl, 3-methoxyphenyl, 2,3,4-trimethoxyphenyl, 3,4,5-trimethoxyphenyl, 2,4-dimethoxyphenyl, 3,5-dimethoxyphenyl, 2-methyl-3-methoxyphenyl, 2-morpholinophenyl, 2-methoxy-4-methylphenyl, 2-dimethylaminophenyl, 4-dimethylaminophenyl, or

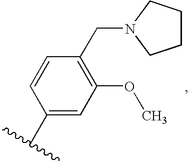

particularly phenyl; 2-methoxyphenyl; 3-methoxyphenyl; 2,3,4-trimethoxyphenyl; 3,4,5-trimethoxyphenyl; 2,4-dimethoxyphenyl; 3,5-dimethoxyphenyl; 2-methyl-3-methoxyphenyl; 2-morpholinophenyl; 2-methoxy-4-methylphenyl; 2-dimethylaminophenyl; or 4-dimethylaminophenyl.

In a further embodiment, the invention provides [symbol] -modulating compounds of Structural Formula (IX):

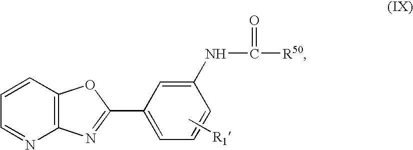

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

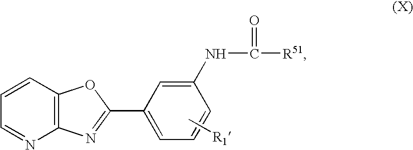

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In certain embodiments, R⁵¹ is selected from pyrazolyl, thiazolyl, oxazolyl, pyrimidinyl, furyl, thienyl, pyridyl, isoxazolyl, indolyl, benzopyrazolyl, benzothiazolyl, benzoxazolyl, quinoxalinyl, benzofuranyl, benzothienyl, quinolinyl, benzoisoxazolyl, benzotriazinyl, triazinyl, naphthyl, or

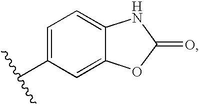

and wherein R⁵¹ is optionally substituted. In certain such embodiments, R⁵¹ is selected from pyrazolyl, thiazolyl, oxazolyl, pyrimidinyl, indolyl, pyrazinyl, triazinyl, or

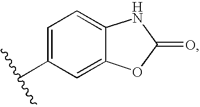

and R⁵¹ is optionally substituted.

In another aspect, the invention provides, [symbol] -modulating compounds of Structural Formula (XI):

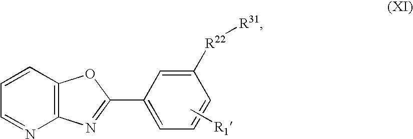

or a salt thereof, wherein:

R₁′ is selected from H or optionally substituted C₁-C₃ straight or branched alkyl;

R²² is selected from —NR²³—C(O)—, —NR₁′—S(O)₂—, —NR₁—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′CR₁′—CR₁′R—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In certain embodiments, R²² is selected from —C(O)—NH—, —NH—, or —C(O)—NH—CH₃.

In certain embodiments, such as when R²² is selected from —C(O)—NH—, —NH—, or —C(O)—NH—CH₃, R³¹ is selected from optionally substituted phenyl, benzothiazolyl, quinoxalinyl, or benzoxazolyl.

In yet another aspect, the invention provides [symbol] modulating compounds of Structural Formula (XII):

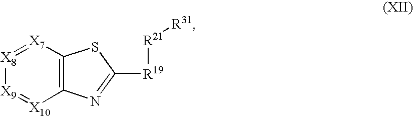

or a salt thereof wherein:
each of X₇, X₈, X₉ and X₁₀ is independently selected from N, CR²⁰, or CR₁′, wherein:
each R₁′ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

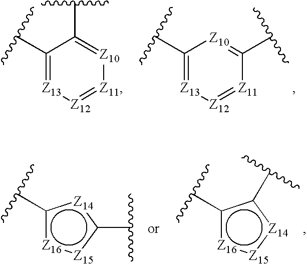

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that when R¹⁹ is

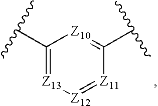

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

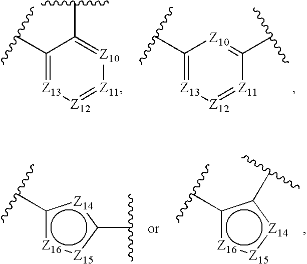

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

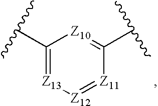

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In certain embodiments, R²¹ is —NH—C(O)— and R¹⁹ is selected from:

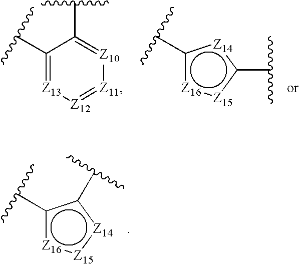

In certain embodiments, R¹⁹ is selected from optionally substituted phenyl, optionally substituted pyridyl, optionally substituted thienyl or optionally substituted furyl.

In certain embodiments, R¹⁹ is

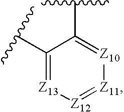

wherein each of Z₁₀, Z₁₁, Z₁₂ and Z₁₃ is independently selected from CR²⁰ or CR₁′; and

R²¹ is selected from —NH—C(O)—, —NH—C(O)—CH(CH₃)—O—, —NH—C(O)—CH₂—O—, or —NH—S(O)₂—CH₂—CH₂—; and

R³¹ is selected from an optionally substituted aryl, or an optionally substituted heteroaryl.

In certain such embodiments, R³¹ is optionally substituted with 1 to 3 substituents independently selected from —OCH₃, —CH₃, —N(CH₃)₂, phenyl, phenoxy, 3,4-dioxymethylene, fluoro, or another solubilizing group. Suitable examples of R³¹ include unsubstituted quinolinyl, 2,4-dimethoxyphenyl, 3,4-dimethoxyphenyl, 3,5-dimethoxyphenyl, 3,4,5-trimethoxyphenyl, 2,3,4-trimethoxyphenyl, 2-dimethylaminophenyl, 3-dimethylaminophenyl, 4-dimethylaminophenyl, 3,5-dimethylphenyl, 3,5-difluorophenyl, 3-trifluoromethoxyphenyl, unsubstituted quinoxalinyl, unsubstituted benzopyrimidinyl,

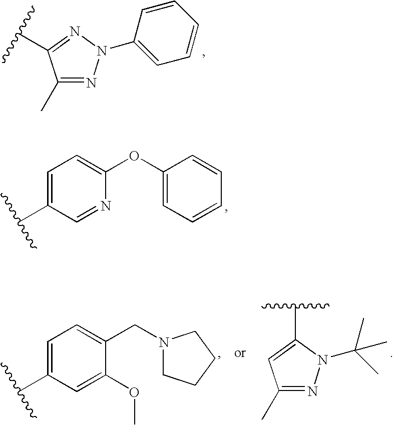

In certain such embodiments, R³¹ is not phenyl-substituted furyl.

In certain embodiments, R¹⁹ is selected from

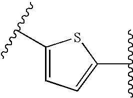

or

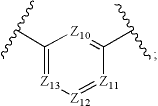

each of Z₁₀, Z₁₁, Z₁₂ and Z₁₃ is independently selected from CR²⁰, or CR₁′;

R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)—; and

R³¹ is selected from an optionally substituted phenyl, an optionally substituted naphthyl, or an optionally substituted heteroaryl.

In certain such embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

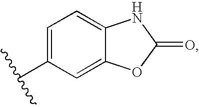

and R³¹ is optionally substituted (e.g., optionally substituted with up to three substituents independently selected from —OCH₃, —CH₃, —N(CH₃)₂, —O-phenyl, or another solubilizing group). Suitable examples of R³¹ include unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

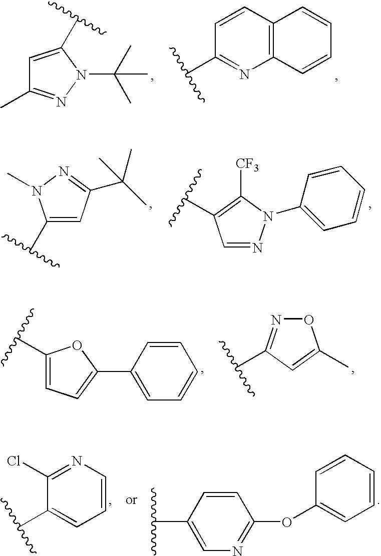

In certain embodiments, one or more of the following conditions applies:

when Xs is N, R²¹ is —NH—C(S)—NH—, and R¹⁹ is phenyl, R³¹ is not 2-methoxy-5-nitrophenyl, 2-S-methylphenyl or 2-acetylphenyl;

when Xs is N, R²¹ is —NH—S(O)—, and R¹⁹ is phenyl, R³¹ is not thiadiazole-substituted thienyl or 4-methylsulfonylphenyl;

when X₈ is N, R²¹ is —NH—CO—, and R¹⁹ is phenyl, R³¹ is not 2,4-difluorophenyl, pyridyl-substituted thienyl, 3,4-dichlorophenyl, 4-t-butylphenyl or 3-benzyloxyphenyl;

when X₉ is N, R²¹ is —NH—C(O)— and R¹⁹ is

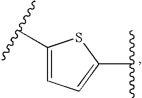

R³¹ is not 2,3,4-trimethoxyphenyl or 3,5-dimethoxyphenyl; and

when X₉ is N, R²¹ is —NH—C(O)— and R¹⁹ is phenyl, R³¹ is not 3,5-dimethoxyphenyl.

In a further embodiment, the invention provides compounds of Structural Formula (XIII):

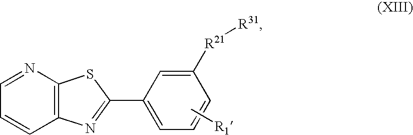

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′—CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In certain embodiments, R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from a monocyclic or bicyclic aryl or a monocyclic or bicyclic heteroaryl, and comprises a solubilizing group substituent.

In certain embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

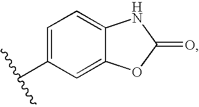

and R³¹ is optionally substituted.

In certain embodiments, R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, particularly when R²¹ is —NH—C(O)—, R³¹ is selected from R³¹ is selected from unsubstituted phenyl, 3-methoxyphenyl, 4-methoxyphenyl, 2,3 dimethoxyphenyl, 2,4-dimethoxyphenyl, 2,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

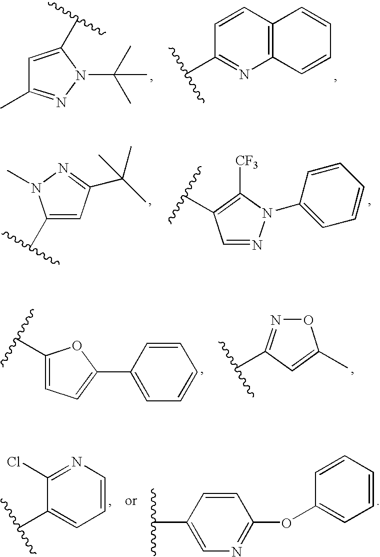

In one aspect, the invention provider [symbol] -modulating compounds of Structural Formula (XIV):

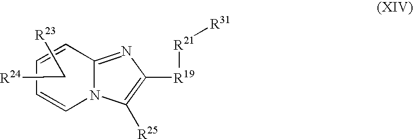

or a salt thereof, wherein:

each of R²³ and R²⁴ is independently selected from H, —CH₃ or a solubilizing group;

R²⁵ is selected from H or a solubilizing group; and

R¹⁹ is selected from:

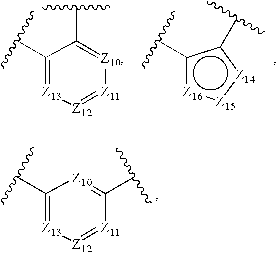

or

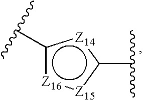

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

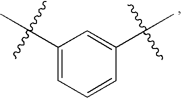

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In certain embodiments, each of R²³ and R²⁴ is independently selected from H, —CH₃ or a solubilizing group;

R²⁵ is selected from H, or a solubilizing group; and

R¹⁹ is selected from:

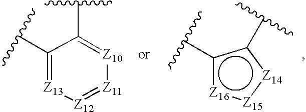

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, R³¹ is not 2,4-dimethoxyphenyl.

Typically, R²⁵ is selected from H, —CH₂—N(CH₃)₂, or

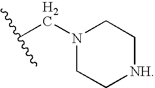

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In certain embodiments, each of R²³ and R²⁴ is independently selected from H, —CH₃ or a solubilizing group;

R²⁵ is selected from H, or a solubilizing group; and

R¹⁹ is selected from:

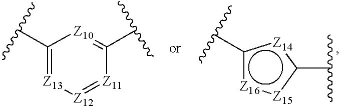

wherein:
each Z₁₀, Z₁₁, Z₁₂ and Z₁₃ is independently selected from N, CR²⁰, or CR₁′; and
each Z₁₄, Z₁₄ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ is N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is phenyl, at least one of R²³, R²⁴, or R²⁵ is a solubilizing group and wherein said compound is not 2-chloro-N-[3-[3-(cyclohexylamino)imidazo[1,2-a]pyridin-2-yl]phenyl]-4-nitrobenzamide.

Typically, R²⁵ is selected from H, —CH₂—N(CH₃)₂, or

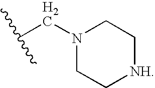

Typically, R²³ and R²⁴ are H.

Typically, R¹⁹ is selected from phenyl, pyridyl, thienyl or furyl, particularly optionally substituted phenyl. Preferably, a phenyl is optionally substituted with:

b) up to three —O—CH₃ groups; or

b) one —N(CH₃)₂ group.

In another aspect, the invention provides [symbol] modulating compounds of Structural Formula (XV):

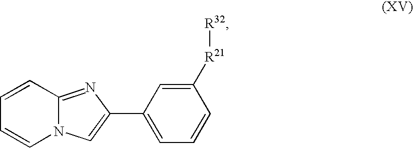

or a salt thereof wherein:

R²¹ is selected from —NR₁′—C(O)—, —NR₁′—S(O)—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

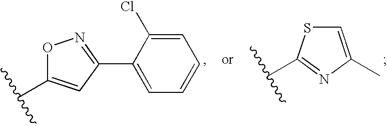

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

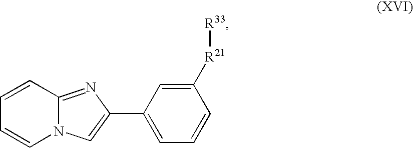

or a salt thereof; wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁═CR₁—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′—R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—NR₁′—C(═N—CN)—NR₁′—, —NR₁′—C(O)—CR₁′—R₁′—CR₁′R₁′—, —NR₁′—C(S)—NR₁′—CR₁′R₁′—CR₁′—R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In certain embodiments, R²¹ is selected from —NR²²—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′, —NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′R—CR₁′R₁′—, —NR₁′—C(O)—CR₁′═CR₁′—CR₁′R₁′—, —NR₁′—C(═N—CN)—NR₁′—, or —NR₁′—C(O)—CR₁′R₁′—; and

each R₁′ is independently selected from H or optionally substituted C₁-C₃ straight or branched alkyl;

R²² is an optionally substituted C₁-C₃ straight or branched alkyl; and

R³³ is phenyl comprising a solubilizing group substituent, wherein: when R²¹ is —NH—S(O)₂ said phenyl comprises an additional substituent.

In certain embodiments, R²¹ is selected from —NR²²—C(O)—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, or —NR₁′—C(═N—CN)—NR₁′—,

each R₁′ is independently selected from H or optionally substituted C₁-C₃ straight or branched alkyl; and

R²² is an optionally substituted C₁-C₃ straight or branched alkyl.

In certain embodiments, R³³ is optionally substituted on up to three carbon atoms with a substituent independently selected from —O—CH₃, —CH₃, —N(CH₃)₂, —S(CH₃), or CN; or substituted on adjacent carbon atoms with

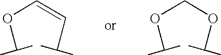

bridging said adjacent carbon atoms.

In a further embodiment, the invention provides [symbol] -modulating compounds of Structural Formula (XVII):

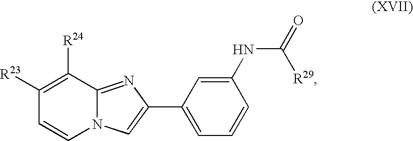

or a salt thereof wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position,

wherein R²⁹ is optionally additionally substituted with a solubilizing group.

In certain embodiments, R²⁹ is phenyl substituted with:

a) three —O—CH₃ groups located at the 2, 3 and 4 positions; or

b) one —N(CH₃)₂ group.

In one aspect, the invention provides [symbol] modulating compounds of Structural Formula (XVIII):

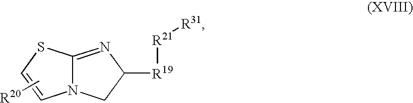

or a salt thereof, wherein:

R¹⁹ is selected from:

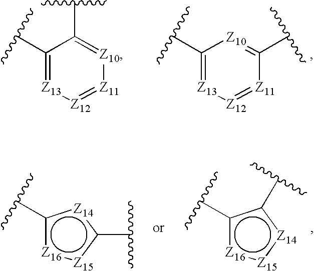

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

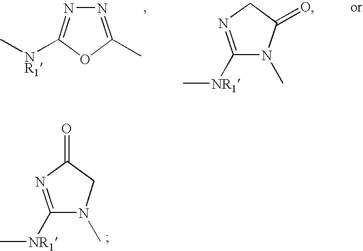

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

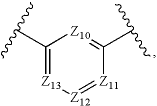

Z₁₀, Z₁₁, Z₁₂ and Z₁₃ are each CH, R²⁰ is H, and R²¹ is —NHC(O)—, R³¹ is not an optionally substituted phenyl.

In certain embodiments, R¹⁹ is selected from:

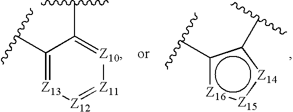

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

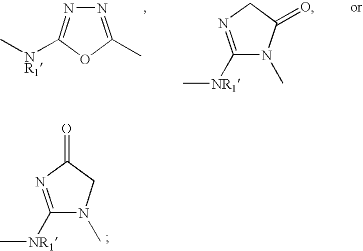

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, compounds of Structural Formula (XVIII) have the formula:

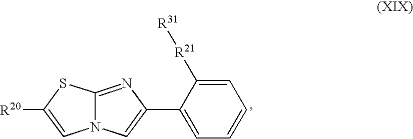

or a salt thereof, wherein

R²⁰ is selected from H or a solubilizing group;

R²¹ is selected from —NH—C(O)—, or —NH—C(O)—CH₂—; and

R³¹ is selected from an optionally substituted monocyclic or bicyclic aryl, or an optionally substituted monocyclic or bicyclic heteroaryl.

Typically, R¹⁹ in compounds of Structural Formula (XVIII) is selected from phenyl, pyridyl, thienyl or furyl, particularly optionally substituted phenyl.

Typically, R²⁰ is selected from H, —CH₂—N(CH₃)₂,

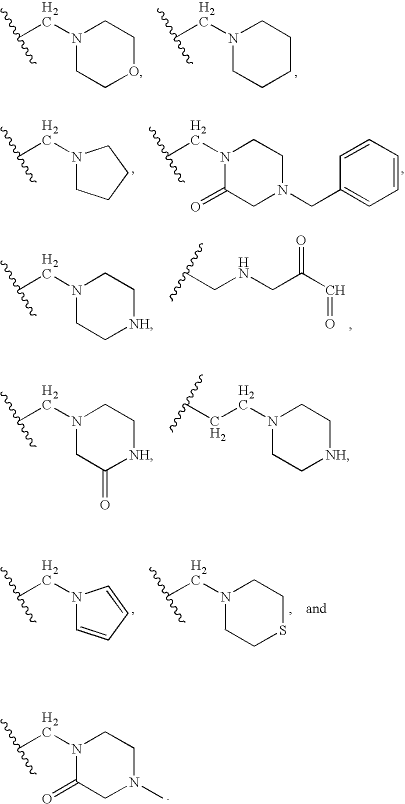

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In certain such embodiments, when R²¹ is —NR₁′—C(O)—, R³¹ is not 4-cyanophenyl or

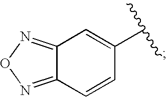

and/or when R²¹ is —NR₁′—S(O)₂—, R³¹ is not 4-methoxyphenyl or 4-t-butylphenyl.

In certain such embodiments, when R¹⁹ is

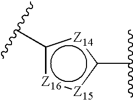

or

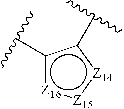

and R²¹ is —NR₁′—C(O)—, R³¹ is not 4-cyanophenyl or

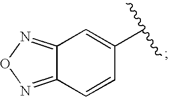

and/or when R¹⁹

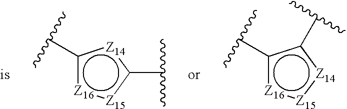

and R²¹ is —NR₁′—S(O)₂—, R³¹ is not 4-methoxyphenyl or 4-t-butylphenyl.

In another aspect, the invention provides [symbol] -modulating compounds of Structural Formula (XX):

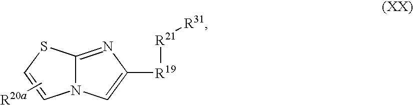

or a salt thereof, wherein

R¹⁹ is selected from:

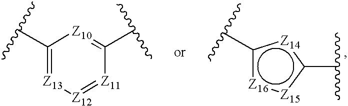

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R^{20a} is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′, —NR₁′—S(O)O—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

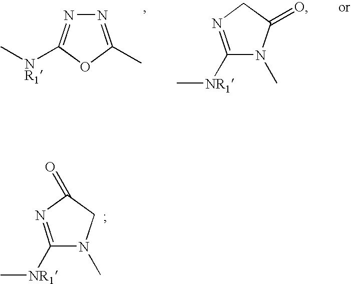

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

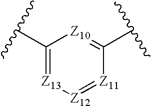

and Z₁₀, Z₁₁, Z₁₂ and Z₁₃ are each CH, R²⁰ is a solubilizing group.

Typically, R¹⁹ in compounds of Structural Formula (XX) is selected from phenyl, pyridyl, thienyl or furyl, particularly optionally substituted phenyl.

Typically, R^{20a} is selected from H, —CH₂—N(CH₃)₂,

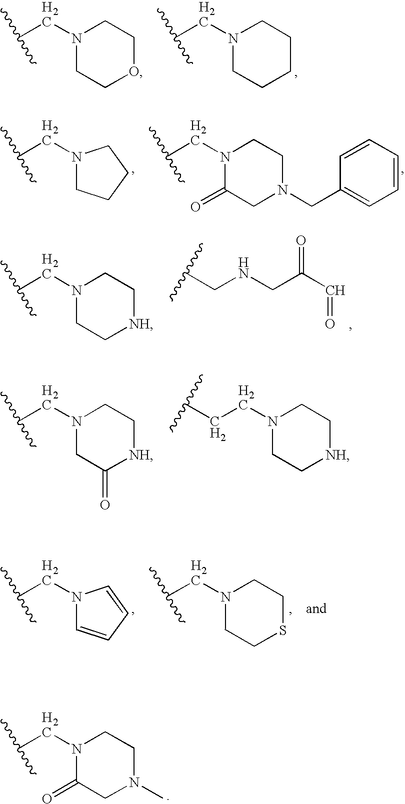

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In yet another aspect, the invention provides [symbol] -modulating compounds of Structural Formula (XXI):

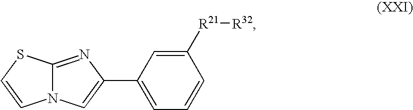

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(—N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

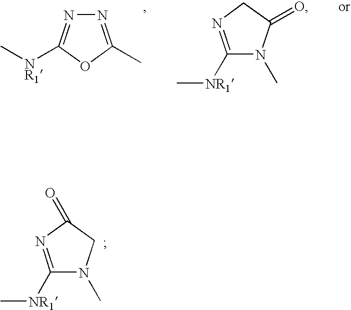

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³ is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In certain embodiments, R³² is selected from pyrrolyl, pyrazolyl, pyrazinyl, furyl, pyridyl, pyrimidinyl, or thienyl, and R³² is optionally substituted and is optionally benzofused.

In certain embodiments, R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

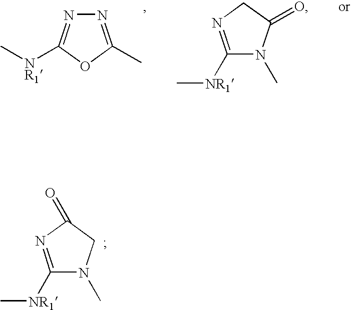

wherein
each R₁′ is independently selected from H or optionally substituted C₁-C₃ straight or branched alkyl; and

R²³ is selected from benzofuryl, methylfuryl, benzothienyl, pyridyl, pyrazinyl, pyrimidinyl, pyrazolyl, wherein said methyfuryl, pyridyl, pyrazinyl, pyrimidinyl or pyrazolyl is optionally benzofused and wherein R³² is optionally substituted or further substituted.

In a further aspect, the invention provides [symbol] -modulating compounds of Structural Formula (XXII):

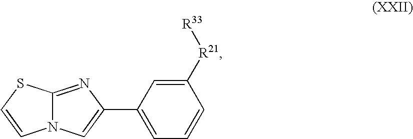

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—C(O)—CR₁′R₁′—O—, —NR₁′—S(O)₂—CR₁′R₁′—, —NR₁′—S(O)₂—CR₁′R₁′—CR₁′R₁′—,

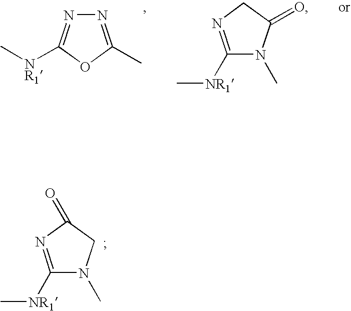

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

Preferably, R²¹ is selected from —NH—C(O)— or —NH—C(O)—CH₂—.

In one aspect, the invention provide [symbol] -modulating compounds of Structural Formula (XXII):

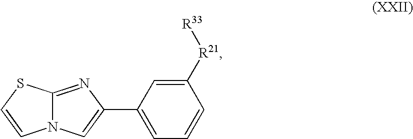

or a salt thereof wherein:

R²¹ is selected from —NH—C(O)—, or —NH—C(O)—CH₂—; and

R³³ is phenyl substituted with

e) one —N(CH₃)₂ group;

f) one CN group at the 3 position;

g) one —S(CH₃) group; or

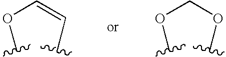

bridging the 3 and 4 positions.

In another aspect, the invention provides [symbol] -modulating compounds of Structural Formula (XXIII):

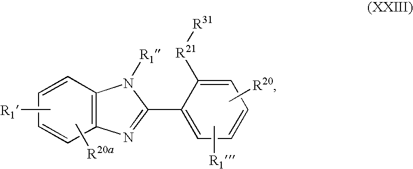

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)₂—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

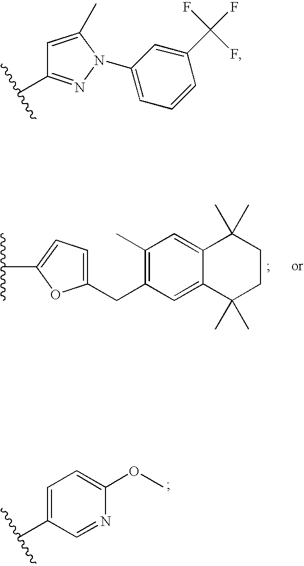

when R²¹ is —NH—C(O)— and each of R²⁰, R^{20a}, R₁′, R₁″ and R₁′″ is hydrogen, R³¹ is not

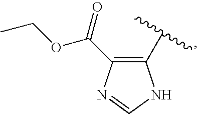

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;
when R²¹ is —NH—C(O)—, R₁″ is methyl; and each of R²⁰, R^{20a}, R₁′ and R₁′″ is hydrogen, R³¹ is not 2-methylaminophenyl,

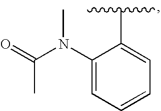

or

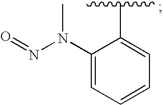

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;
when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R²¹ is hydrogen or —CH₂—N(CH₂CH₃)₂, and each of R²⁰, R₁′, R₁″ and R₁′″ is hydrogen, R³¹ is not

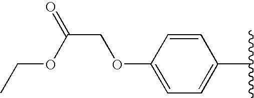

or

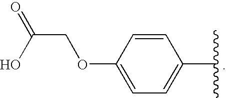

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3-substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In a particular aspect, the invention provides [symbol] -modulating compounds of Structural Formula (XXIII):

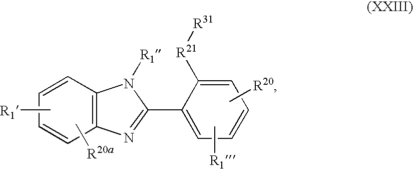

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl,
wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In yet another aspect, the invention provides [symbol] -modulating compounds of Structural Formula (XXIV):

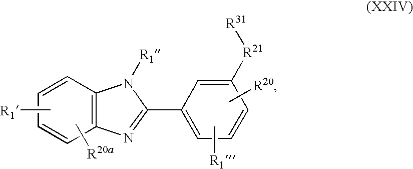

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂O—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that
when R²¹ is —NH—C(O)—CH₂—, R³¹ is not 2-methylphenyl, or 3,4-dimethoxyphenyl;
when R²¹ is —NH—C(O)—CH═CH—, R³¹ is not 2-chlorophenyl;
when R²¹ is —NH—C(O)—NH—, R³¹ is not unsubstituted benzimidazolyl;
when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;
when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;
when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;
when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

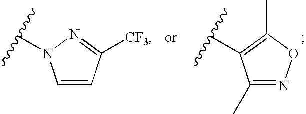

when R²¹ is —NH—C(O)—CH₂—, R₁′ is methyl, and each of R²⁰, R^{20a}, R₁″, and R₁″ is hydrogen, R³¹ is not unsubstituted phenyl;
when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;
when R²¹ is —NH—C(O)—CH(CH₂CH₃)—, and each of R²⁰, R^{20a}, R₁′, R₁″, and R₁′″ is hydrogen, R³¹ is not unsubstituted phenyl;
when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] modulating compounds of Structural Formula (XXIV):

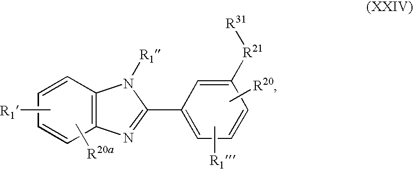

or a salt thereof; wherein:
each R²⁰ and R^{20a} is independently selected from H or a solubilizing group and at least one of R²⁰ and R^{20a} is a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R—, —NR₁′—C(═N—CN)—NR₁′—, —NR₁′—C(O)—CR₁′R₁′—O—, —NR₁′—C(O)—CR₁′R₁′—CR₁′R′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and
R³¹ is selected from an optionally substituted monocyclic or bicyclic aryl, or an optionally substituted monocyclic or bicyclic heteroaryl.

In certain embodiments, when R²¹ is —NH—C(O)—CH₂—, R³¹ is not 2-methylphenyl; or 3,4-dimethoxyphenyl; when R²¹ is —NH—C(O)—CH═CH—, R³¹ is not 2-chlorophenyl; and/or when R²¹ is —NH—C(O)—NH—, R³¹ is not unsubstituted benzimidazolyl.

In a further aspect, the invention provides [symbol] -modulating compounds of Structural Formula (XXV):

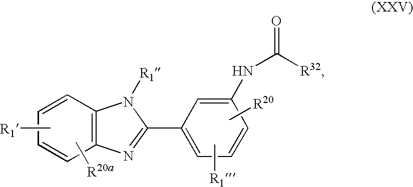

or a salt thereof, wherein:
each R²⁰ and R^{20a} is independently selected from H or a solubilizing group, wherein at least one of R²⁰ and R^{20a} is a solubilizing group;
each R₁′, R₁″ and R₁′″ is independently selected from H or optionally substituted C₁-C₃ straight or branched alkyl; and
R³² is an optionally substituted phenyl.

In certain embodiments, R³² is selected from 3,4-dimethoxyphenyl, 2,6-dimethoxyphenyl, or 2,4-dimethoxyphenyl; wherein R³² is further optionally substituted with a solubilizing group.

In certain embodiments, R³² is not unsubstituted thienyl; unsubstituted phenyl; 2-methylphenyl; 4-fluorophenyl; 4-methoxyphenyl; 4-methylphenyl; 3,4-dioxyethylenephenyl; 3-acetylamino-4-methylphenyl; 3-[(6-amino-1-oxohexyl)amino]-4-methylphenyl; 3-amino-4-methylphenyl; 3,5-dimethoxyphenyl; 3-halo-4-methoxyphenyl; 3-nitro-4-methylphenyl; or 4-propoxyphenyl.

In one aspect, the invention provides [symbol] -modulating compounds of Structural Formula (XXVI):

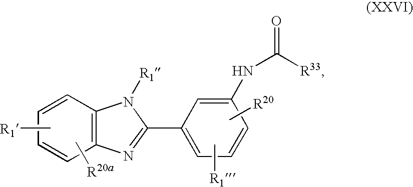

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl; and
R³³ is selected from an optionally substituted heteroaryl or an optionally substituted bicyclic aryl, with the provisos that
when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

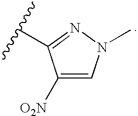

In a particular aspect, the invention provides [symbol] -modulating compounds of Structural Formula (XXVI):

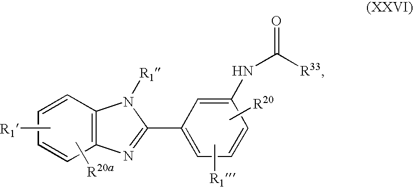

or a salt thereof, wherein:
each R²⁰ and R^{20a} is independently selected from H or a solubilizing group, wherein at least one of R²⁰ or R^{20a} is a solubilizing group;
each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] -modulating compounds of Structural Formula (XXVII):

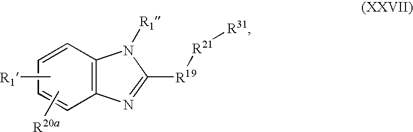

wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

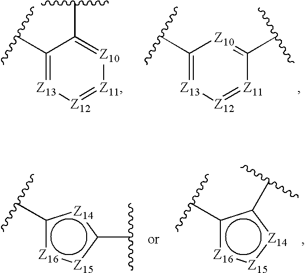

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

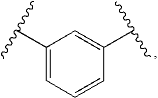

R³¹ is not unsubstituted pyridyl, 26-dimethoxyphenyl, 3,4,5-trimethoxyphenyl or unsubstituted furyl.

In a particular aspect, the invention provides [symbol] -modulating compounds of Structural Formula (XXVII):

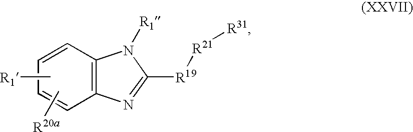

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

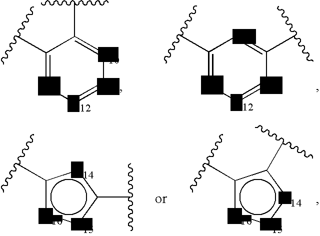

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)—, —NR₁—CR₁′R₁′—C(O)—N′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—, and R¹⁹ is thiazolyl, R³¹ is not optionally substituted phenyl or optionally substituted pyridyl;
when R²¹ is —NH—C(O)—CH₂—, and R¹⁹ is pyrazolyl, R³¹ is not unsubstituted indolyl or unsubstituted phenyl;
when R²¹ is —NH—C(O)—CH₂—, and R¹⁹ is

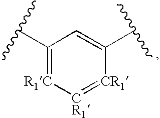

R³¹ is not 2-methylphenyl or 3,4-dimethoxyphenyl;
when R²¹ is —NH—C(O)—CH═CH—, and R¹⁹ is

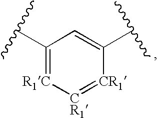

R³¹ is not 2-chlorophenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;
when R²¹ is —NH—C(O)—NH—, and R¹⁹ is

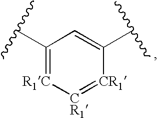

R³¹ is not unsubstituted benzimidazolyl;
when R²¹ is —NH—, and R¹⁹ is pyrazolyl, R³¹ is not unsubstituted pyridyl;
when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;
when R²¹ is —NH— and R¹⁹ is pyridyl, oxadiazolyl or thiadiazolyl, R³¹ is not unsubstituted phenyl, 3-methoxyphenyl or 4-methoxyphenyl;
when R²¹ is —NH—C(O)— and R¹⁹ is thiazolyl or pyrimidinyl, R³¹ is not unsubstituted phenyl;
when R²¹ is —NH—C(O)— and R¹⁹ is

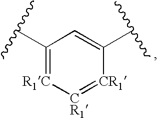

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;
when R²¹ is —NH—C(O)— and R¹⁹ is

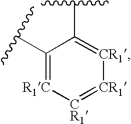

R³¹ is not 3,5-dinitrophenyl, 4-butoxyphenyl,

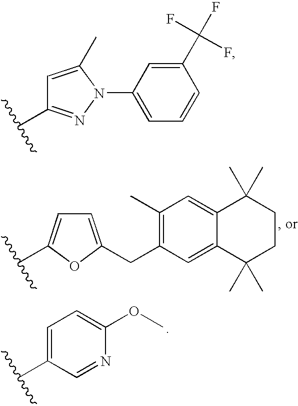

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In certain embodiments, when R²¹ is —NH—C(O)—, R¹⁹ is not

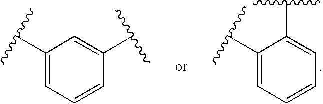

In certain embodiments, when R²¹ is —NH—C(O)—, R¹⁹ is not optionally substituted pyrazolyl, thiazolyl, thienyl, pyrrolyl or pyrimidinyl; when R²¹ is —NH—C(O)—CH2— or —NH—C(O)—NH—, R¹⁹ is not pyrazolyl; and/or when R²¹ is —NH—, R¹⁹ is not optionally substituted pyridyl, thiazolyl, pyrazolyl, thiadiazolyl, or oxadiazolyl.

In a more particular aspect, the invention provides [symbol] -modulating compounds of Structural Formula (XXVII):

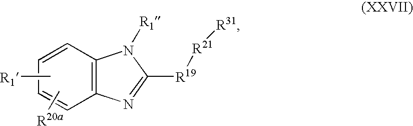

or a salt thereof, wherein:
each R²⁰ and R^{20a} is independently selected from H or a solubilizing group;

each R₁′ and R₁″ is independently selected from H or optionally substituted C₁-C₃ straight or branched alkyl;

R¹⁹ is selected from:

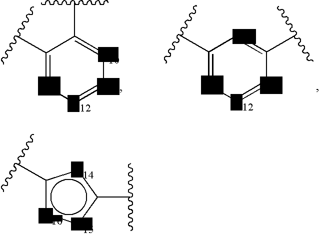

or

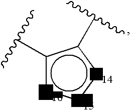

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
one to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—C(O)—CH₂—, and R¹⁹ is pyrazolyl, R³¹ is not unsubstituted indolyl or unsubstituted phenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;
when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;
when R^{20a} is a solubilizing group, R¹⁹ is thienyl and R²¹ is —NH—C(O)—, R^{3l} is not unsubstituted phenyl;
when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and
when R²¹ is —NH—C(O)— and R¹⁹ is thiazolyl or pyrimidinyl, R³¹ is not unsubstituted phenyl.

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In yet another aspect, the invention provides compounds of Structural Formula (XXVIII):

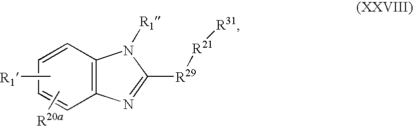

or a salt thereof; wherein:
each R²⁰ and R^{20a} is independently selected from H or a solubilizing group;
each R₁′ and R₁″ is independently selected from H or optionally substituted C₁-C₃ straight or branched alkyl;
R²⁹ is selected from:

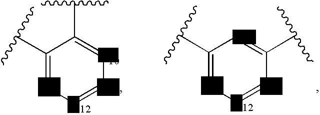

wherein:
each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁—; and

R³¹ is selected from an optionally substituted monocyclic or bicyclic aryl, or an optionally substituted monocyclic or bicyclic heteroaryl.

In certain embodiments, R³¹ is optionally substituted phenyl, such as 3-methoxyphenyl, 3,4-dimethoxyphenyl, 3,4,5-trimethoxyphenyl, or 4-dimethylaminophenyl.

In certain embodiments, R²¹ is —NH—C(O)—.

In preferred embodiments, R²¹ is —NH—C(O)— and R³¹ is an optionally substituted phenyl, such as 3-methoxyphenyl, 3,4-dimethoxyphenyl, 3,4,5-trimethoxyphenyl, or 4-dimethylaminophenyl.

In a further aspect, such as when the [symbol] modulator is a sirtuin inhibitor, the invention provides novel sirtuin-modulating compounds of Formula (VI):

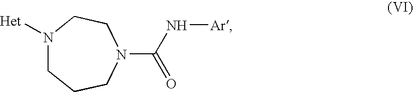

or a salt thereof, wherein:

Het is an optionally substituted heterocyclic aryl group; and

Ar′ is an optionally substituted carbocyclic or heterocyclic aryl group.

In certain embodiments, Het comprises one N heteroatom and 1 to 2 additional heteroatoms independently selected from N, O or S, such as oxazolopyridyl.

In certain embodiments, Ar′ is selected from optionally substituted phenyl, benzothiazolyl, or benzoxazolyl. When Ar′ is substituted phenyl, typically it is substituted with 1 to 3 substituents independently selected from halo, methyl, O-methyl, S-methyl or N(CH₃)₂, morpholino, or 3,4 dioxymethylene.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

In the compounds described above, bivalent groups disclosed as possible values for variables can have either orientation, provided that such orientation results in a stable molecule. Preferably, however, the left hand side of a bivalent group (e.g., —NR₁′—C(O)—) is attached to a bivalent arylene or heteroarylene group (e.g., R¹⁹) and the right hand side of a bivalent group is attached to a monovalent aryl group (e.g., R³¹).
compounds of the invention having hydroxyl substituents, unless otherwise indicated, also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

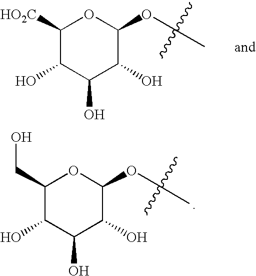

These groups are generally cleavable to —OH by hydrolysis or by metabolic (e.g., enzymatic) cleavage.

In certain embodiments, the compounds of the invention exclude one or more of the species disclosed in Tables 4-6. In certain such embodiments, the compounds of the invention exclude compound 7.
[symbol] -modulating compounds of the invention advantageously [symbol] modulate the level and/or activity of a protein.

Separately or in addition to the above properties, certain [symbol] -modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A ring (e.g., 5- to 7-membered ring) or cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuryl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OC₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}ONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}, —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR_{c}═CR^{a}R^{b}; CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}—R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

A hydrogen-bond donating group is a functional group having a partially positively-charged hydrogen atom (e.g., —OH, —NH₂, —SH) or a group (e.g., an ester) that metabolizes into a group capable of donating a hydrogen bond.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R^{a}—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C6-C14) aryl, phenyl, naphthyl, (C7-C20) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)ₙ—C(O)—R^{b}, —(CH₂)ₙ—O—(CH₂)ₙ—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or R^{b} moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula: —(CH₂)ₙ—R¹⁰⁰—N(R¹⁰¹)(R¹⁰¹), wherein:

n is selected from 0, 1 or 2;
R¹⁰⁰ is selected from a bond, —C(O)—, or —O(CH₂)ₙ; and
each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;
c.

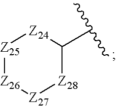

d.

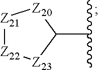

e.

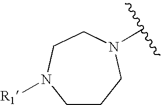

f. both R¹⁰¹ moieties are taken together with the nitrogen atom to which they are bound to form a ring of the structure

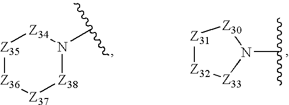

or

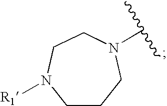

or
g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′;
   wherein:

each Z is independently selected from —O—, —S—, —NR₁′—, or —C(R⁵⁰)(R⁵⁰)—,

wherein:
at least three of Z₂₀, Z₂₁, Z₂₂, and Z₂₃ are —C(R⁵⁰)(R⁵⁰)—;
at least three of Z₂₄, Z₂₅, Z₂₆, Z₂₇, and Z₂₈ are —C(R⁵⁰)(R⁵⁰)—;
at least four of Z₃₀, Z₃₁, Z₃₂, and Z₃₃ are —C(R⁵⁰)(R⁵⁰)—; and
at least four of Z₃₄, Z₃₅, Z₃₆, Z₃₇, and Z₃₈ are —C(R⁵⁰)(R⁵⁰)—;

each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;

each R⁵⁰ is independently selected from R₁′, halo, CN, OH, O—(C₁-C₄ straight or branched alkyl), N(R₁′)(R₁′), ═CR₁′, SR₁′, ═NR₁′, ═NOR₁′, or ═O;

any two suitable non-cyclic R⁵⁰ are optionally bound to one another directly or via a C₁ to C₂ alkylene, alkenylene or alkanediylidene bridge to produce a bicyclic fused or spiro ring, and any

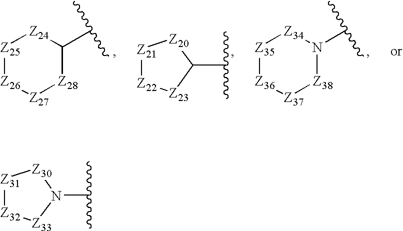

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another preferred embodiment, the solubilizing group is a moiety of the formula: —(CH₂)ₙ—O—R¹⁰¹, wherein n and R¹⁰¹ are as defined above.

In another preferred embodiment, the solubilizing group is a moiety of the formula: —(CH₂)ₙ—C(O)—R₁′, wherein n and R₁′ are as defined above.

In a more preferred embodiment, a solubilizing group is selected from —(CH₂)ₙ—R¹⁰², wherein n is 0, 1 or 2; and R¹⁰² is selected from

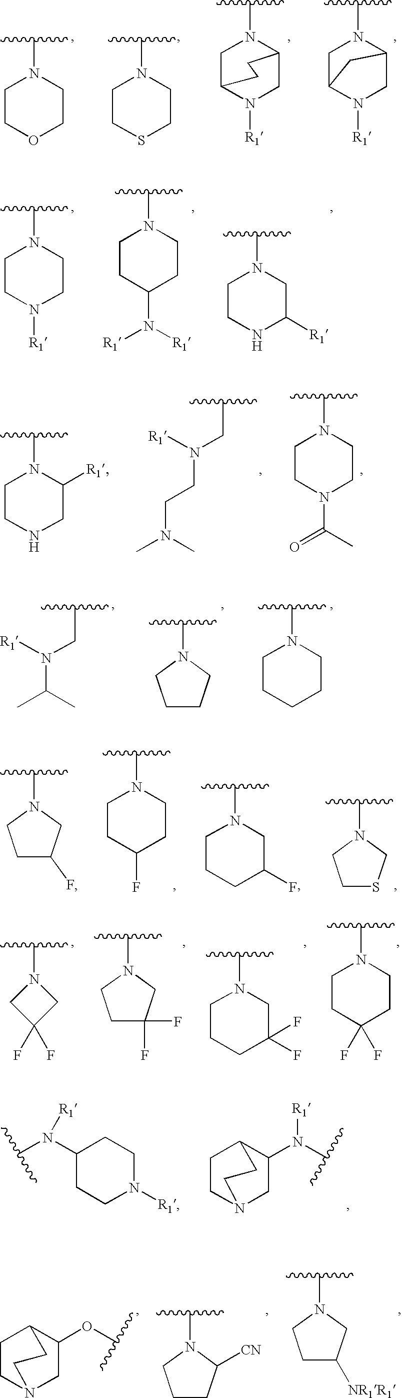

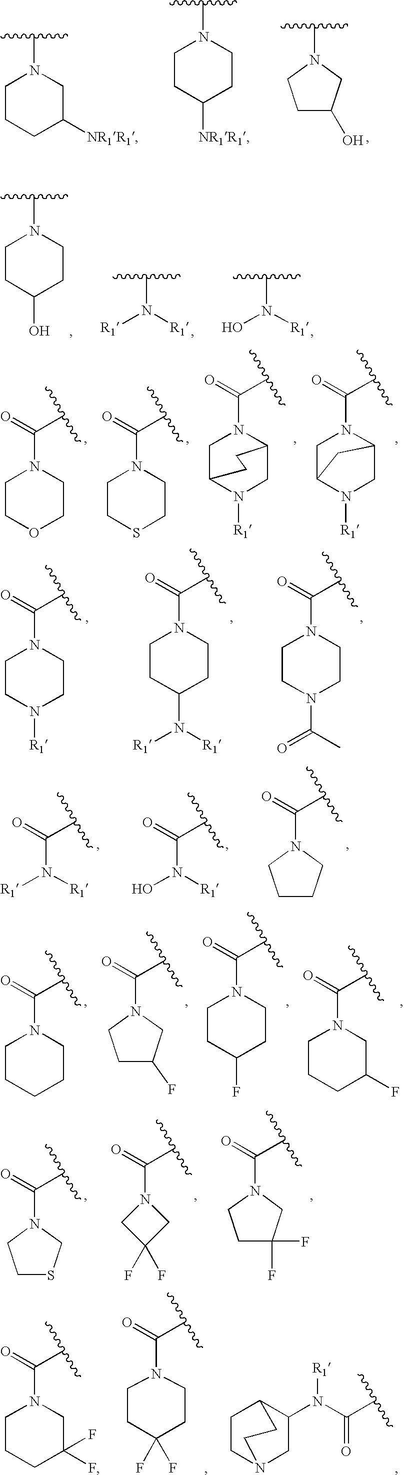

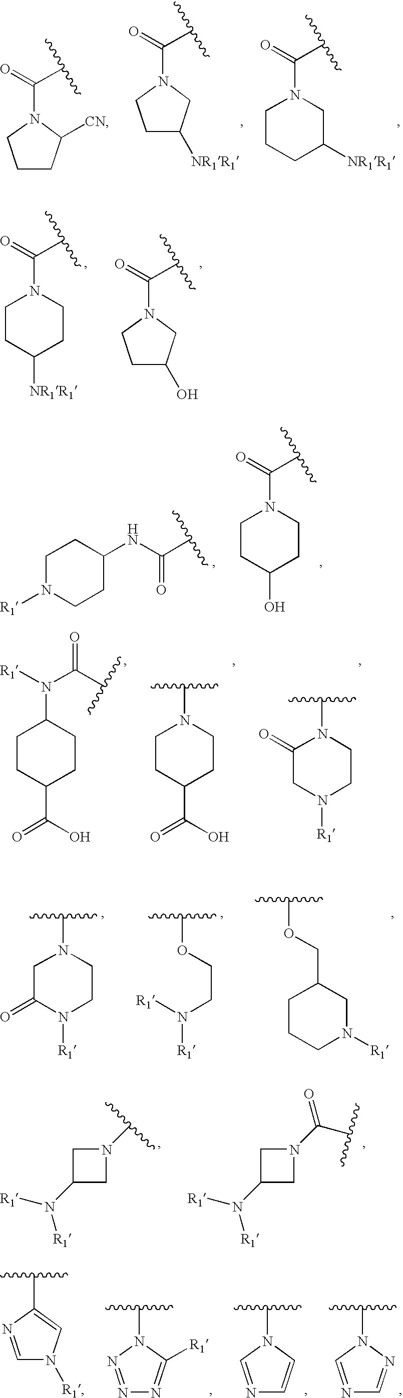

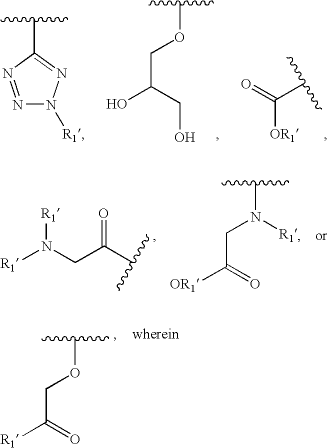

R₁′ are as defined above.

In an even more preferred embodiment, a solubilizing group is selected from 2-dimethylaminoethylcarbamoyl, piperazin-1-ylcarbonyl, piperazinylmethyl, dimethylaminomethyl, 4-methylpiperazin-1-ylmethyl, 4-aminopiperidin-1-yl-methyl, 4-fluoropiperidin-1-yl-methyl, morpholinomethyl, pyrrolidin-1-ylmethyl, 2-oxo-4-benzylpiperazin-1-ylmethyl, 4-benzylpiperazin-1-ylmethyl, 3-oxopiperazin-1-ylmethyl, piperidin-1-ylmethyl, piperazin-1-ylethyl, 2,3-dioxopropylaminomethyl, thiazolidin-3-ylmethyl, 4-acetylpiperazin-1-ylmethyl, 4-acetylpiperazin-1-yl, morpholino, 3,3-difluoroazetidin-1-ylmethyl, 2H-tetrazol-5-ylmethyl, thiomorpholin-4-ylmethyl, 1-oxothiomorpholin-4-ylmethyl, 1,1-dioxothiomorpholin-4-ylmethyl, 1H-imidazol-1-ylmethyl, 3,5-dimethylpiperazin-ylmethyl, 4-hydroxypiperidin-1-ylmethyl, N-methyl(1-acetylpiperidin-4-yl)-aminomethyl, N-methylquinuclidin-3-ylaminomethyl, 1H-1,2,4-triazol-1-ylmethyl, 1-methylpiperidin-3-yl-oxymethyl, or 4-fluoropiperidin-1-yl.

TABLE 4

| COM- | | | ED₅₀ | ED₅₀ | FOLD |
|---|---|---|---|---|---|
| POUND | | | FP | MS | ACT. |
| NO | [M + H]+ | STRUCTURE | ASSAY | ASSAY | MS |
| 1 | | 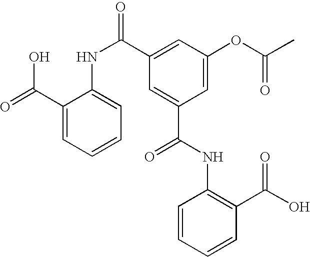 | A | NT | |
| 2 | | 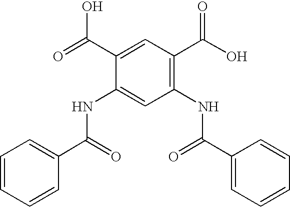 | D | NT | |
| 3 | | 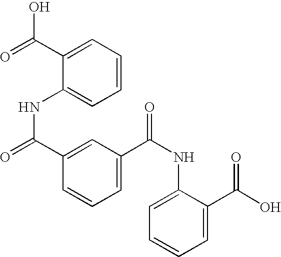 | B | NT | |
| 4 | | 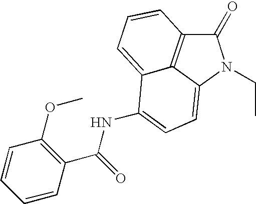 | N/A | NT | |
| 5 | | 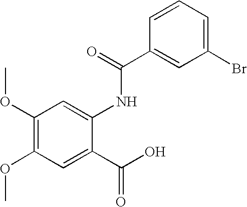 | B | NT | |
| 6 | | 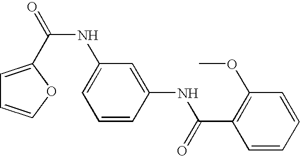 | D | NT | |
| 7 | 346 | 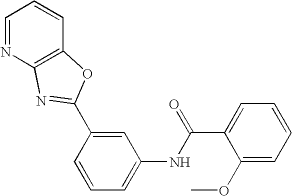 | A | NT | |
| 8 | | 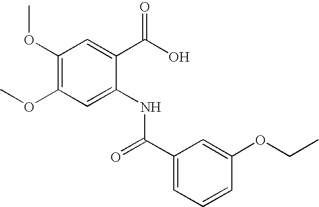 | B | NT | |
| 9 | | 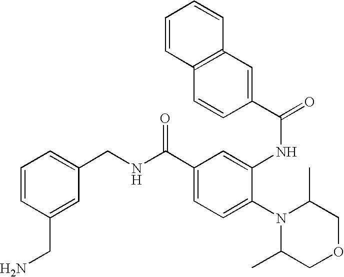 | C | NT | |
| 10 | | 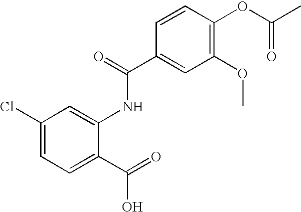 | D | NT | |
| 19 | 409.6 | 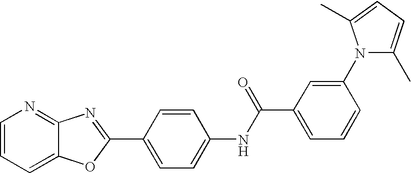 | D | D | C |
| 20 | 401.3 | 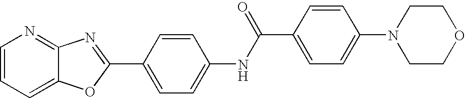 | D | D | C |
| 21 | 399.1 | 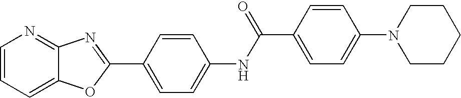 | D | D | C |
| 22 | 414.0 | 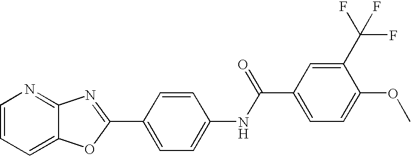 | D | D | C |
| 24 | 359.4 | 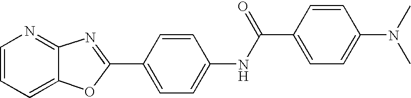 | D | D | C |
| 27 | 376.1 | 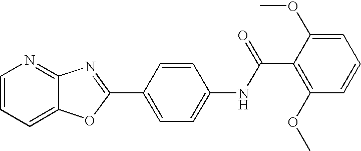 | D | D | C |
| 29 | 385.1 | 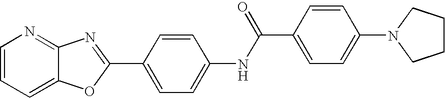 | D | D | C |
| 31 | 360.1 | 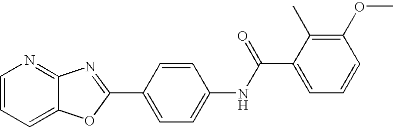 | D | D | C |
| 32 | 400.0 | 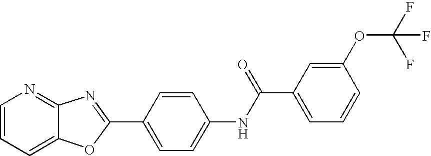 | D | D | C |
| 33 | 376.1 | 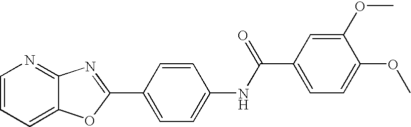 | D | D | C |
| 34 | 406.3 | 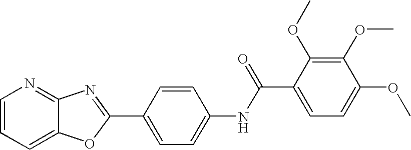 | D | D | C |
| 35 | 346.5 | 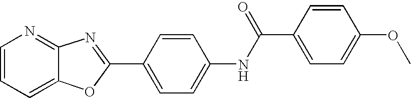 | D | D | C |
| 36 | 376.7 | 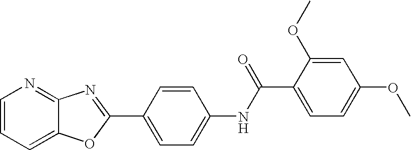 | D | D | C |
| 37 | 316.4 | 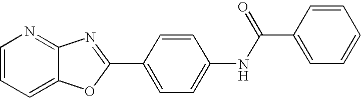 | D | D | C |
| 38 | 401.0 | 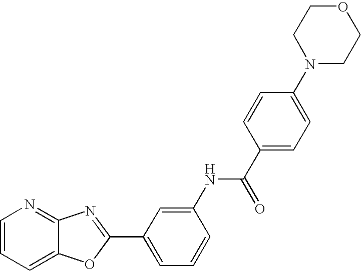 | D | D | C |
| 39 | 399.1 | 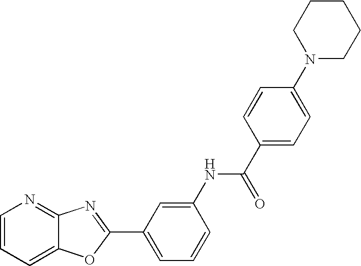 | D | D | C |
| 40 | 414.2 | 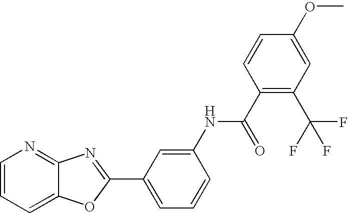 | D | D | C |
| 41 | 414.2 | 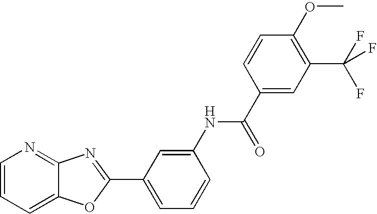 | D | D | C |
| 42 | 359.1 | 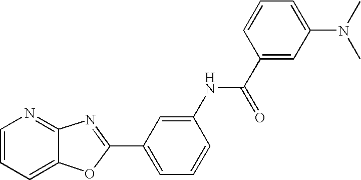 | A | A | B |
| 43 | 359.1 | 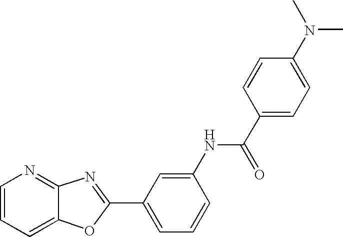 | A | A | B |
| 45 | 341.0 | 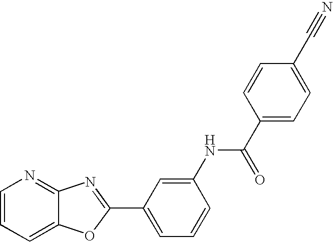 | D | D | C |
| 46 | 376.1 | 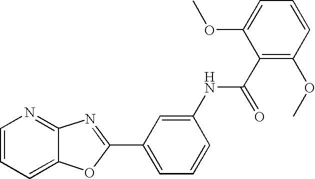 | D | D | C |
| 48 | 406.3 | 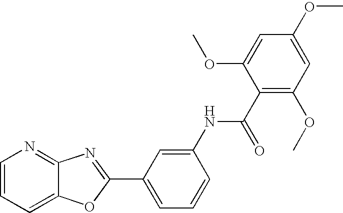 | D | D | C |
| 49 | 360.1 | 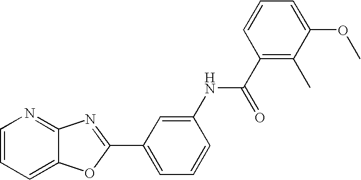 | D | A | B |
| 50 | 400.0 | 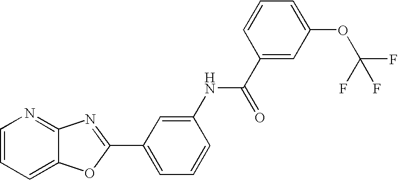 | NT | D | |
| 51 | 360.1 | 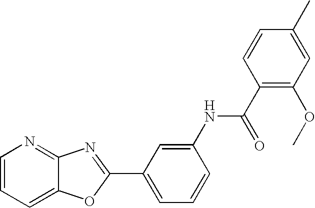 | A | A | B |
| 52 | 376.1 | 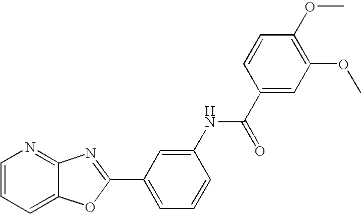 | A | A | B |
| 53 | 406.1 | 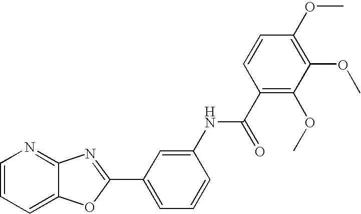 | D | D | C |
| 54 | 346.4 | 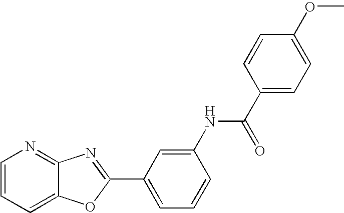 | NT | D | |
| 55 | 376.1 | 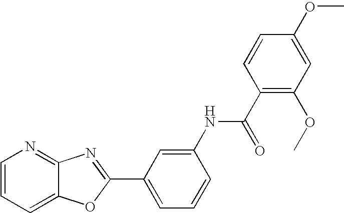 | A | A | B |
| 56 | 316.0 | 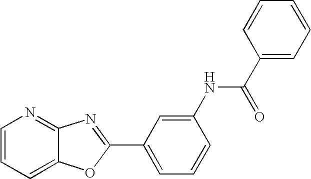 | D | A | B |
| 57 | 407.1 | 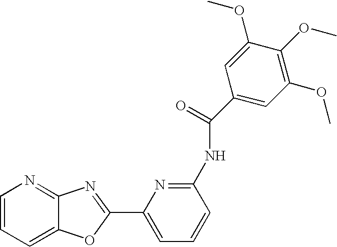 | | A | |
| 58 | 377.1 | 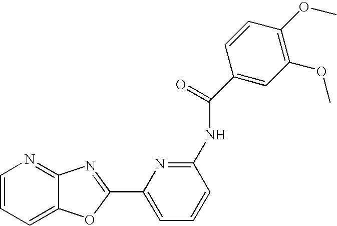 | | NA | |
| 59 | 318.1 | 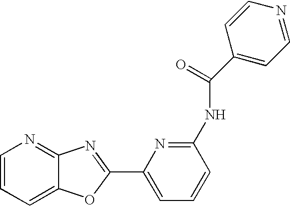 | | NA | |
| 60 | 413.1 | 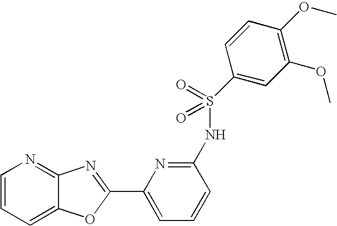 | | A | |
| 61 | 413.1 | 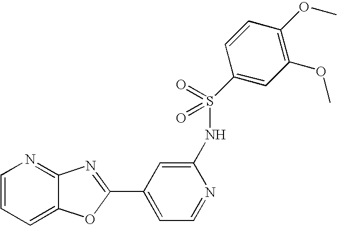 | | NA | |
| 62 | 349.0 | 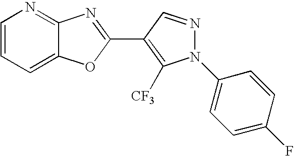 | | NA | |
| 63 | 377.1 | 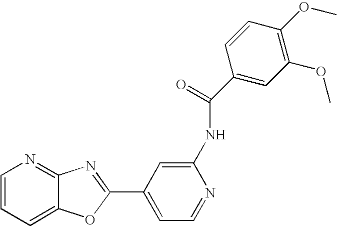 | | A | |
| 64 | 360.1 | 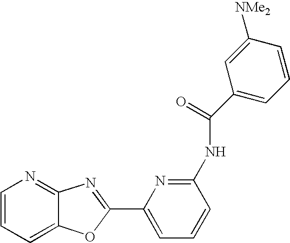 | | A | |
| 65 | 383.0 | 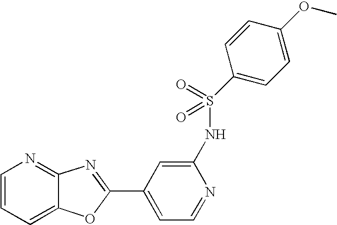 | | NA | |
| 66 | 407 | 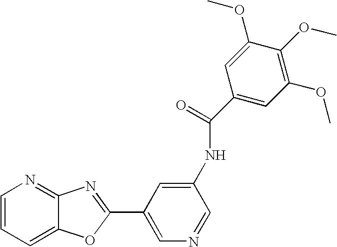 | | A | |
| 67 | 377 | 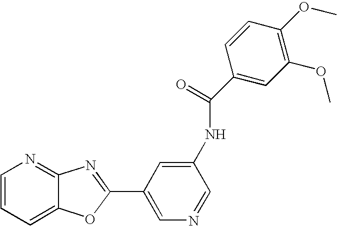 | | A | |
| 68 | 360 | 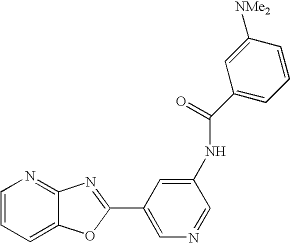 | | A | |
| 69 | 377.1 | 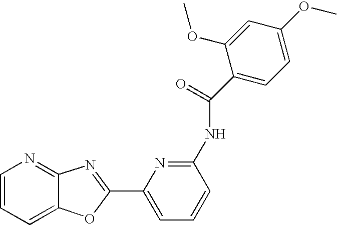 | | A | B |
| 70 | 360.1 | 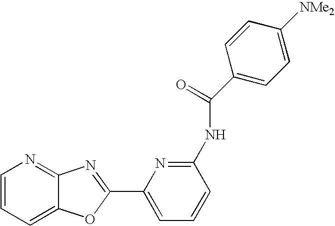 | | A | B |
| 71 | 376.1 | 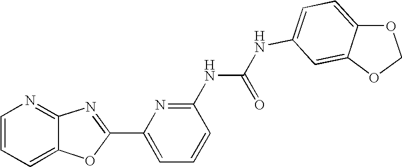 | | A | B |
| 72 | 422.1 | 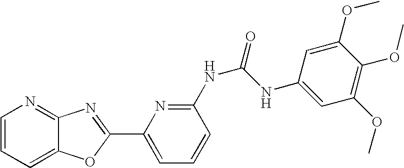 | | NT | |
| 73 | 377 | 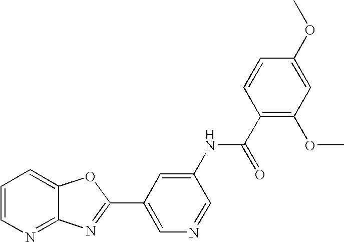 | | NT | |
| 74 | 412 | 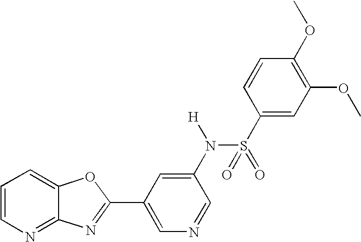 | | D | C |
| 75 | 407.1 | 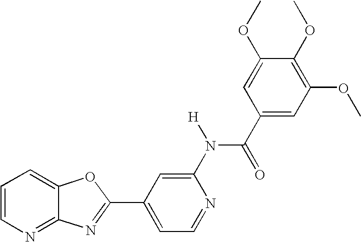 | | A | B |
| 76 | 360.1 | 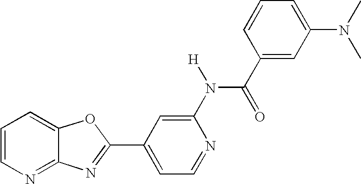 | | A | B |
| 77 | 376.1 | 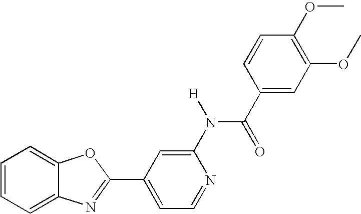 | | A | B |
| 78 | 445.1 | 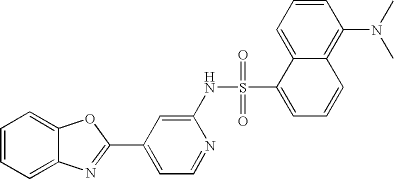 | | | |
| 79 | 338 | 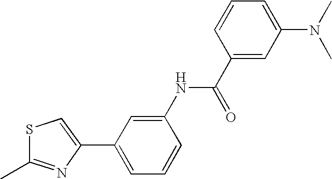 | | D | C |
| 80 | 355 | 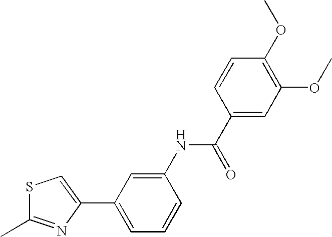 | | A | B |
| 81 | 354.5 | 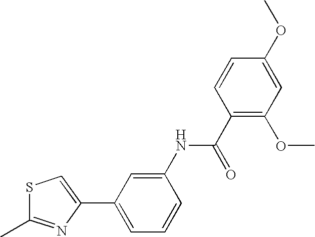 | | A | B |
| 82 | 402 | 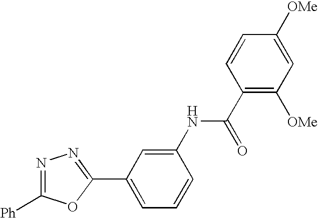 | | C | |
| 83 | 355 | 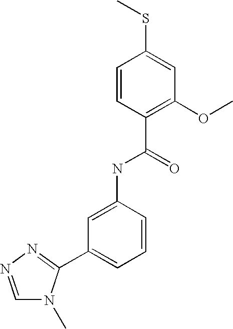 | | A | B |
| 84 | 417 | 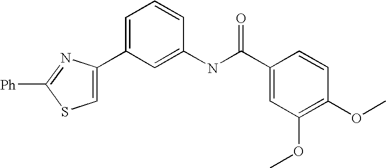 | | A | B |
| 85 | 335.1 | 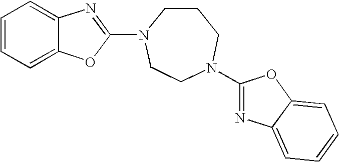 | | D | C |
| 86 | 375.1 | 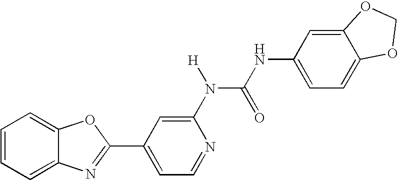 | | D | C |
| 87 | 375.1 | 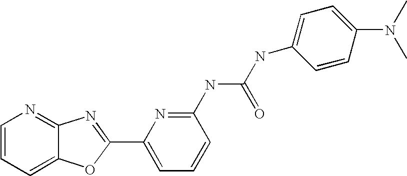 | | D | C |
| 88 | 382.1 | 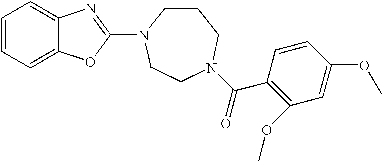 | | D | C |
| 89 | 365.1 | 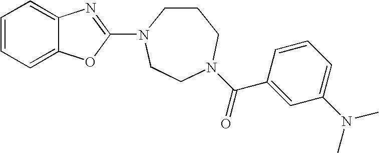 | | D | C |
| 90 | 381.1 | 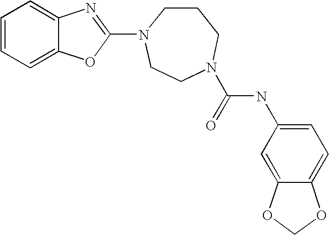 | | | |
| 91 | 412.1 | 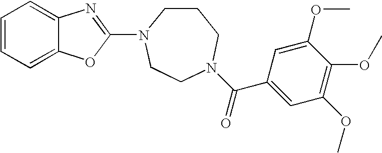 | | D | C |
| 92 | 308.1 | 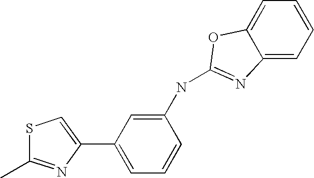 | | A | B |
| 93 | 329.1 | 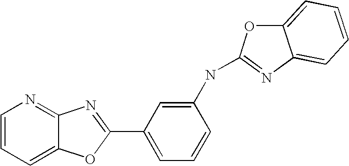 | | A | B |
| 94 | 434.1 | 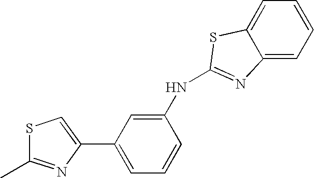 | | A | B |
| 95 | 345.1 | 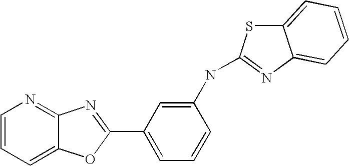 | | A′ | B |
| 96 | 376.1 | 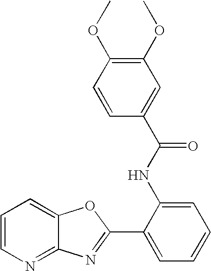 | | A′ | B |
| 97 | 359.1 | 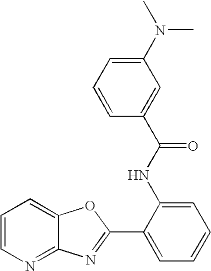 | | A′ | B |
| 98 | 408.1 | 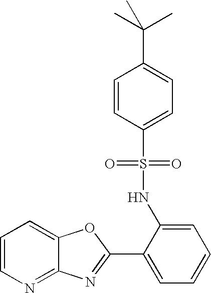 | | D | C |
| 99 | 391 | 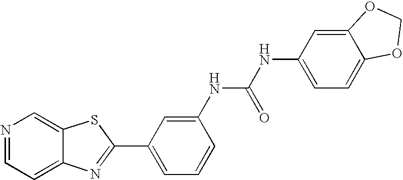 | | A′ | A |
| 100 | 376 | 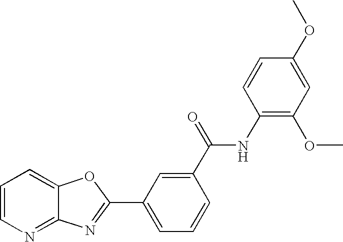 | | A | B |
| 101 | 376 | 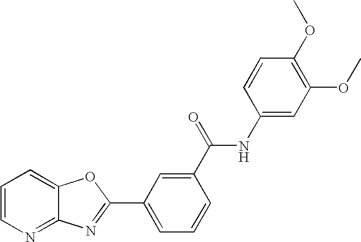 | | A | A |
| 102 | 406 | 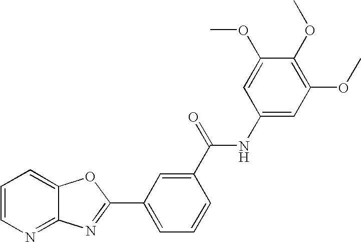 | | A | A |
| 103 | 374 | 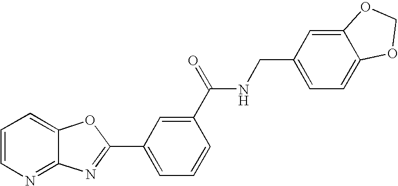 | | D | C |
| 104 | 346.1 | 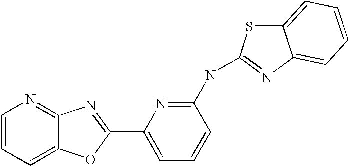 | | A′ | B |
| 105 | 330.1 | 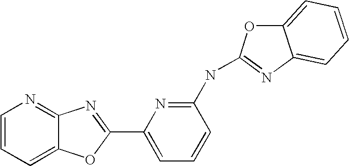 | | A′ | B |
| 106 | 406.1 | 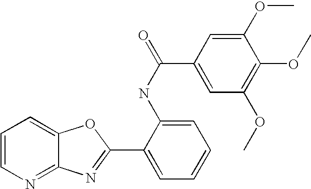 | | A′ | B |
| 107 | 488 | 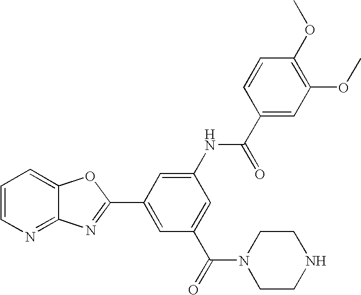 | | A | B |
| 108 | 324 | 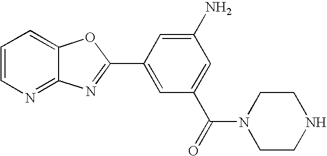 | | NA | |
| 109 | 401 | 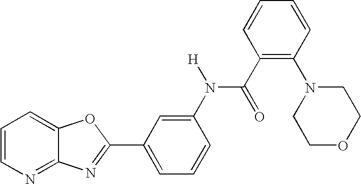 | | A | B |
| 110 | 381 | 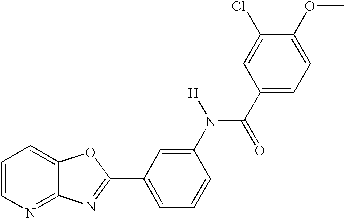 | | C | |
| 111 | 359 | 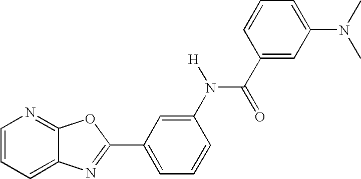 | | A | B |
| 112 | 376 | 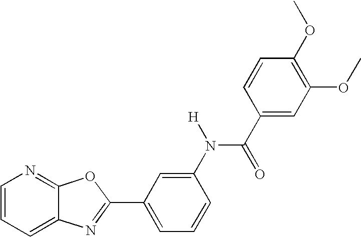 | | A | B |
| 113 | 375 | 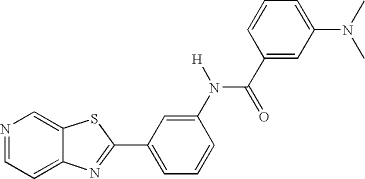 | | A | B |
| 114 | 392 | 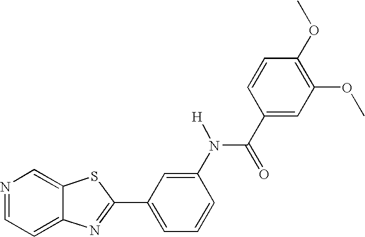 | | A′ | A |
| 115 | 422 | 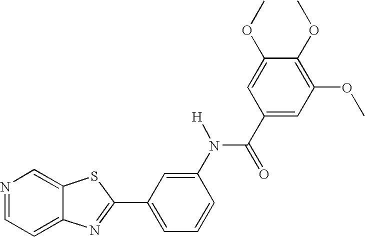 | | A | A |
| 116 | 386 | 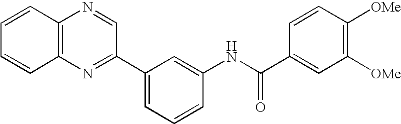 | | C | |
| 117 | 388 | 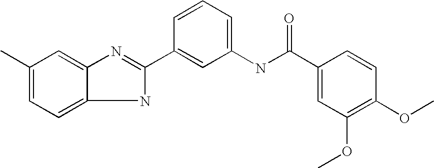 | | A | A |
| 118 | 410 | 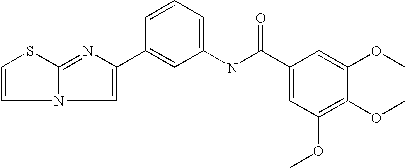 | | A | A |
| 119 | 375 | 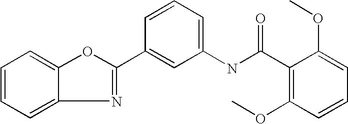 | | A | B |
| 120 | 391 | 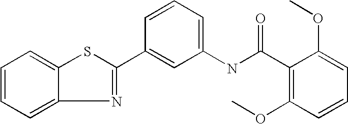 | | NT | |
| 121 | 414 | 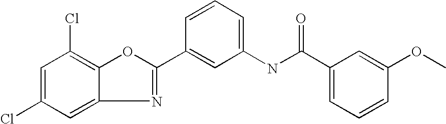 | | D | C |
| 122 | 417 | 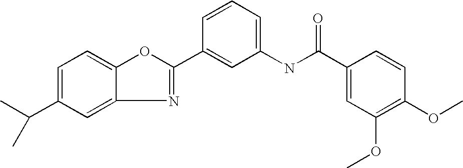 | | C | |
| 123 | 474 | 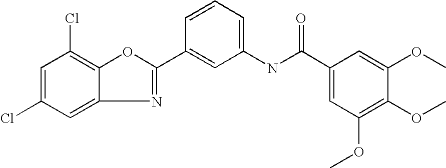 | | NT | |
| 124 | 391 | 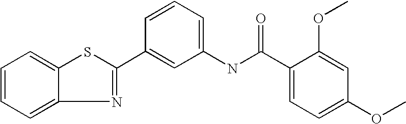 | | A | B |
| 125 | 433 | 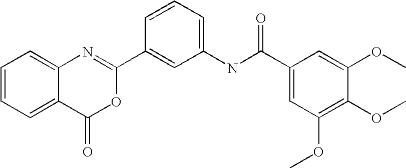 | | B | B |
| 126 | 374 | 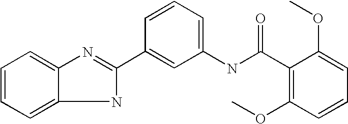 | | A | B |
| 127 | 355 | 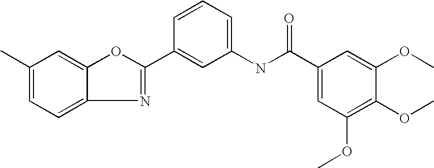 | | A | A |
| 128 | 388 | 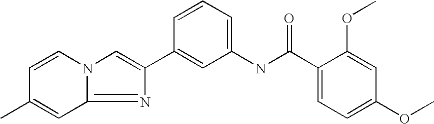 | | A | B |
| 129 | 418 | 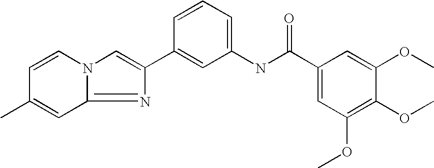 | | A′ | A |
| 130 | 358 | 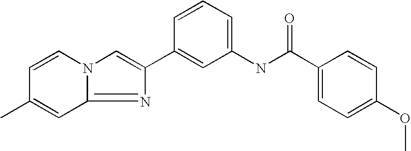 | | A | B |
| 131 | 418 | 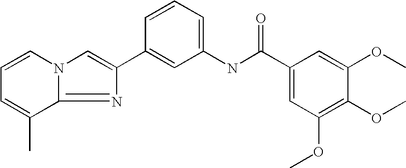 | | A | A |
| 132 | 350 | 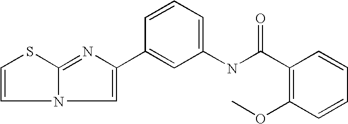 | | A | A |
| 133 | 480 | 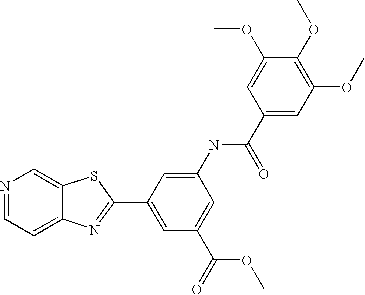 | | A′ | B |
| 134 | 466 | 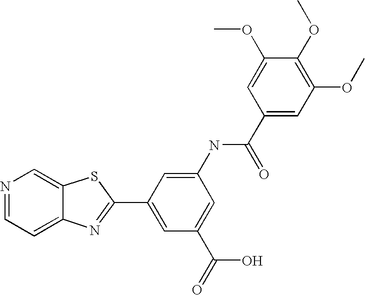 | | A′ | B |
| 135 | 452 | 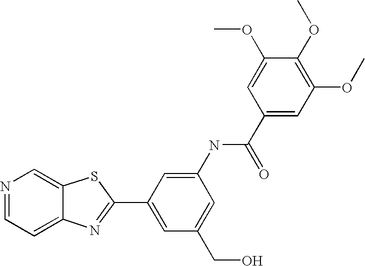 | | A′ | B |
| 136 | 434 | 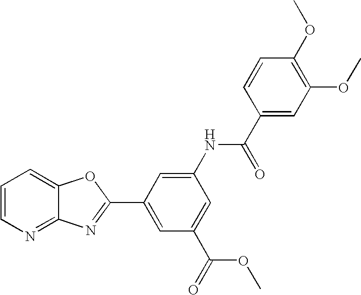 | | D | C |
| 137 | 420 | 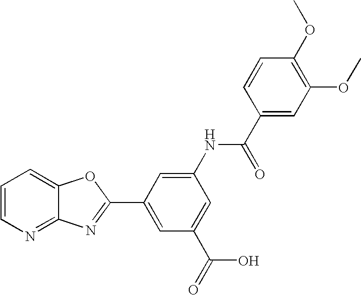 | | D | |
| 138 | 471 | 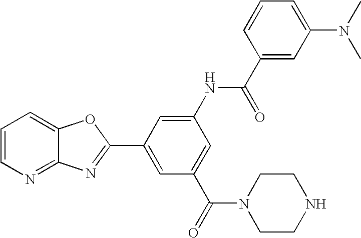 | | A | B |
| 139 | 488 | 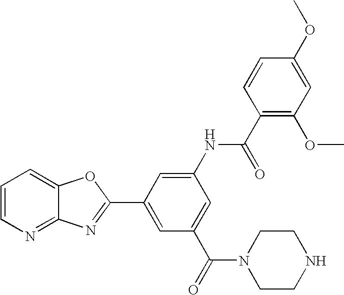 | | A | B |
| 141 | 374.1 | 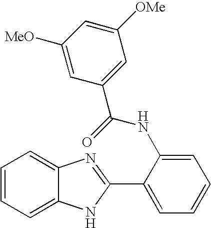 | | A′ | B |
| 142 | 374.1 | 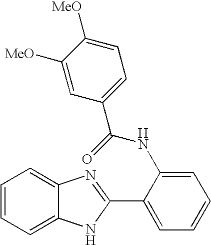 | | A′ | A |
| 143 | 410.1 | 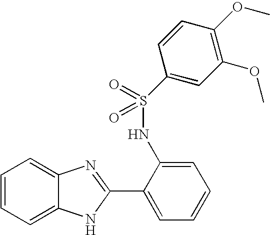 | | D | |
| 144 | 427.1 | 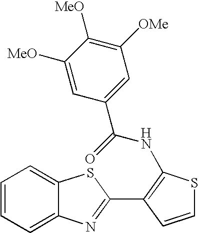 | | D | C |
| 145 | 397.1 | 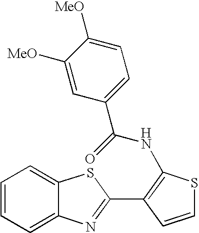 | | A′ | B |
| 146 | 397.1 | 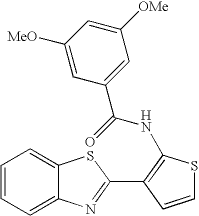 | | A′ | B |
| 147 | 392.1 | 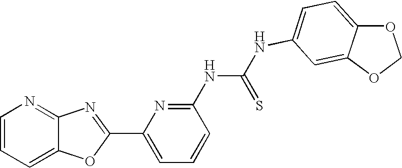 | | D | |
| 148 | 405.2 | 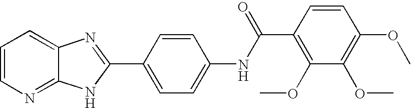 | | D | |
| 149 | 359.0 | 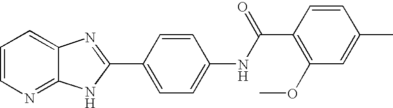 | | A′ | B |
| 150 | 375.0 | 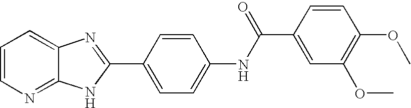 | | A′ | B |
| 151 | 375.0 | 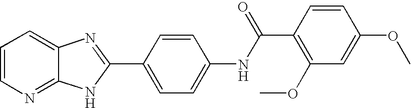 | | D | C |
| 152 | 392.1 | 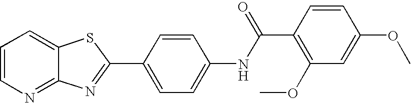 | | D | C |
| 153 | 490 | 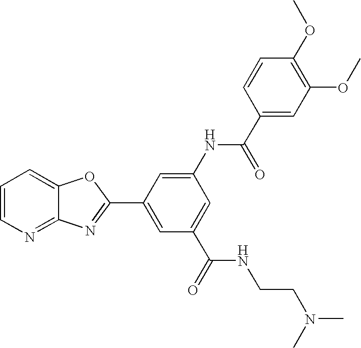 | | A | B |
| 154 | 473 | 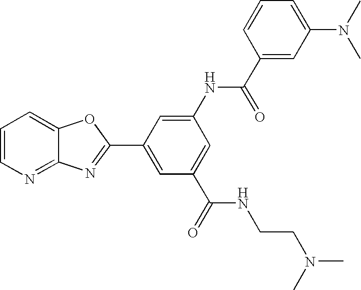 | | C | A |
| 155 | 490 | 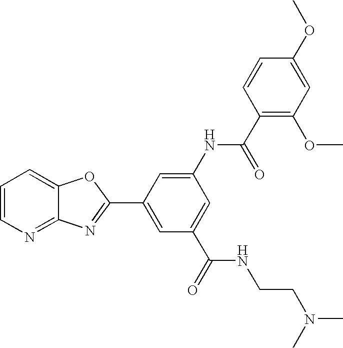 | | A | B |
| 156 | 433 | 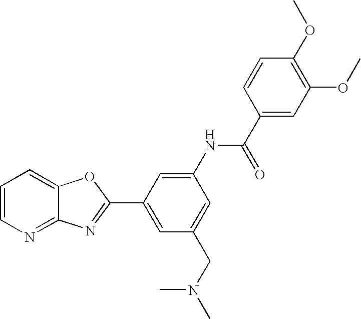 | | A | B |
| 157 | 416 | 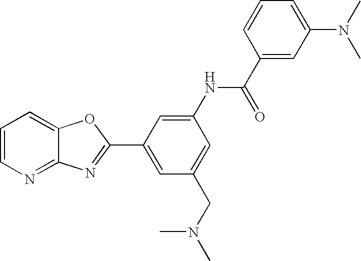 | | D | B |
| 158 | 433 | 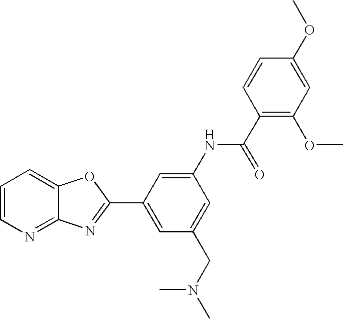 | | A | B |
| 159 | 474 | 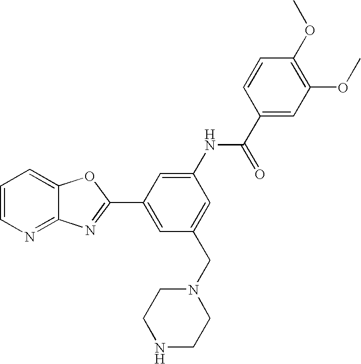 | | A | B |
| 160 | 457 | 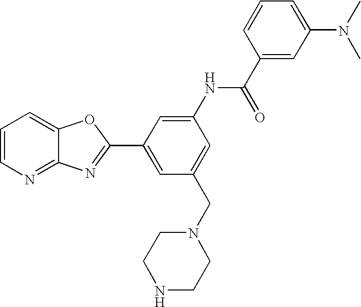 | | B | A |
| 161 | 474 | 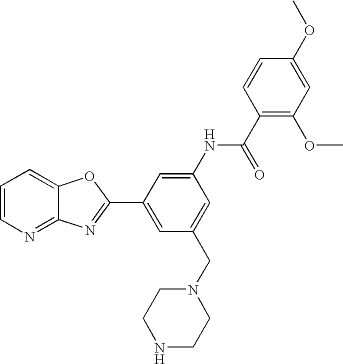 | | A | A |
| 162 | 392.1 | 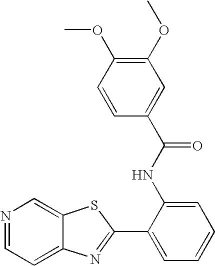 | | A′ | B |
| 163 | 422.1 | 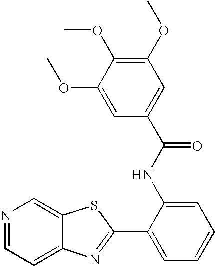 | | A′ | B |
| 164 | 488 | 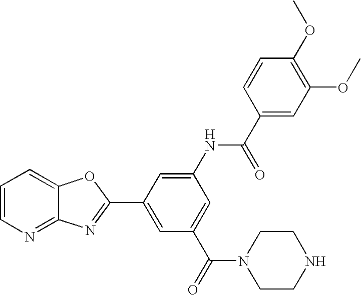 | | A | B |
| 165 | 416 | 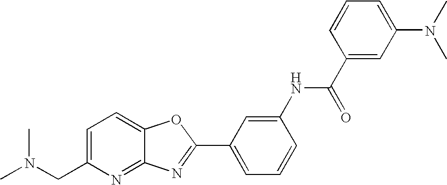 | | D | A |
| 166 | 457 | 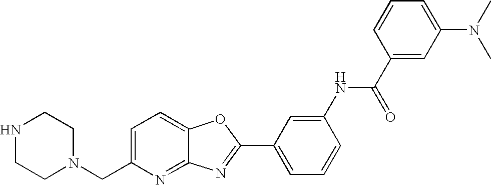 | | A | B |
| 167 | 373 | 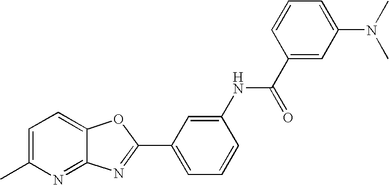 | | A | B |
| 168 | 388.1 | 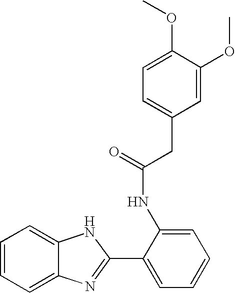 | | NA | C |
| 169 | 343.1 | 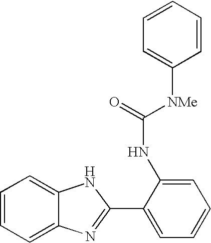 | | A′ | B |
| 174 | 479 | 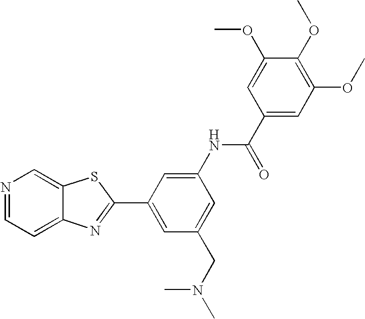 | | B | A |
| 175 | 323.1 | 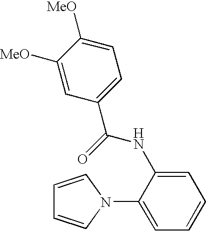 | | B | B |
| 176 | 354.1 | 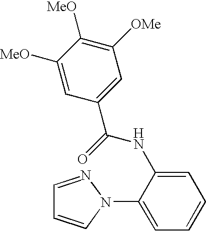 | | B | B |
| 177 | 324.1 | 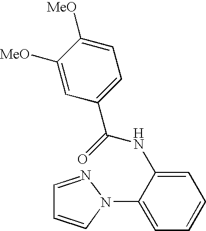 | | D | B |
| 178 | 437 | 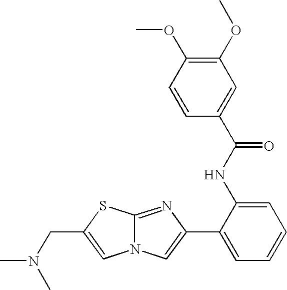 | | A | A |
| 179 | 467 | 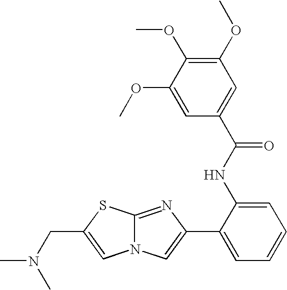 | | A′ | A |
| 180 | 358.0 | 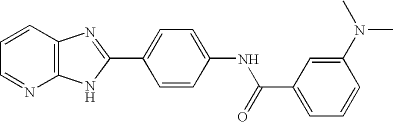 | | A | B |
| 181 | 405.0 | 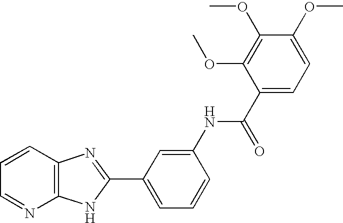 | | A′ | A |
| 182 | 359.0 | 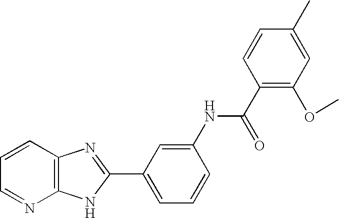 | | A′ | A |
| 183 | 358.0 | 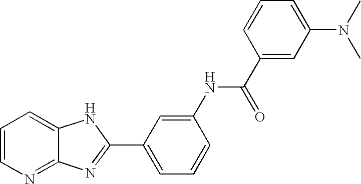 | | A | A |
| 184 | 375.0 | 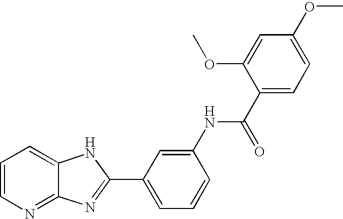 | | A′ | B |
| 185 | 374.9 | 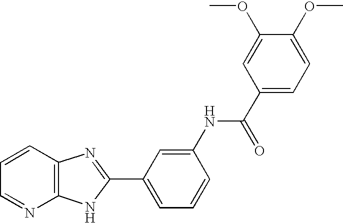 | | A′ | A |
| 186 | 405.3 | 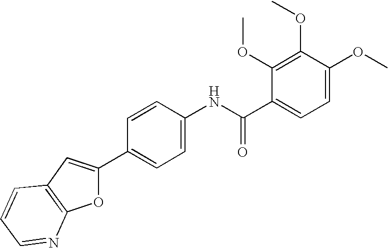 | | NA | C |
| 187 | 358.9 | 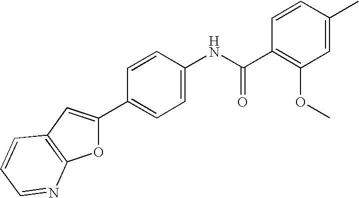 | | A | B |
| 188 | 358.9 | 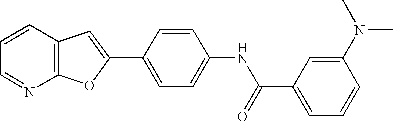 | | NA | C |
| 189 | 375.2 | 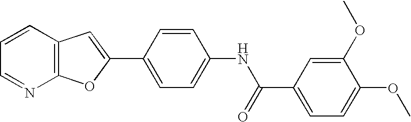 | | NA | C |
| 190 | 375.3 | 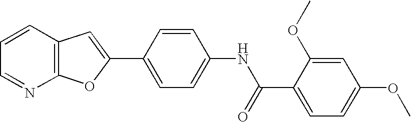 | | A′ | B |
| 191 | 375.0 | 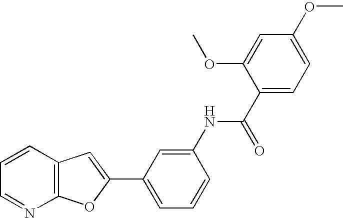 | | A′ | B |
| 192 | 375.0 | 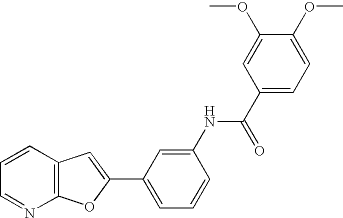 | | A | B |
| 193 | 358.0 | 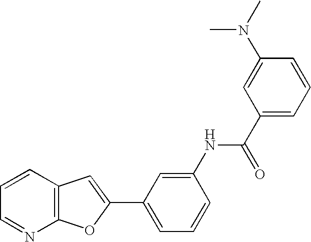 | | NA | C |
| 194 | 422.3 | 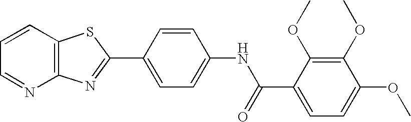 | | NA | C |
| 195 | 375.9 | 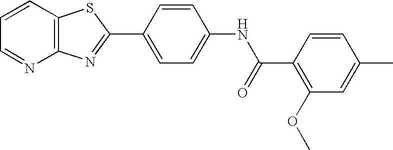 | | NA | C |
| 196 | 374.9 | 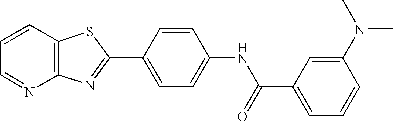 | | A | B |
| 197 | 391.1 | 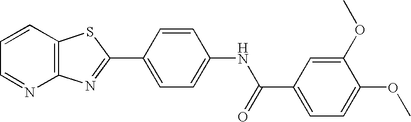 | | A′ | B |
| 198 | 422.4 | 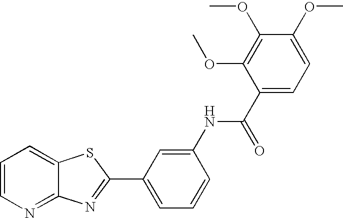 | | A | B |
| 199 | 375.9 | 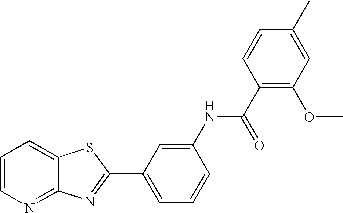 | | A′ | B |
| 200 | 375.4 | 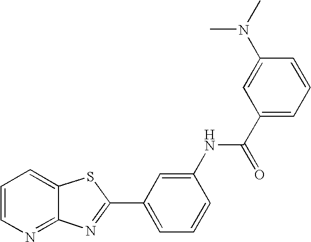 | | A′ | B |
| 201 | 391.9 | 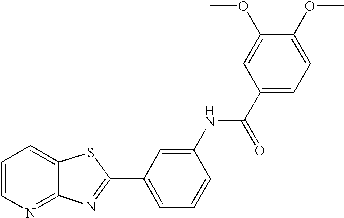 | | A | B |
| 202 | 392.4 | 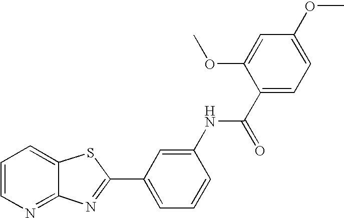 | | A′ | B |
| 203 | 380 | 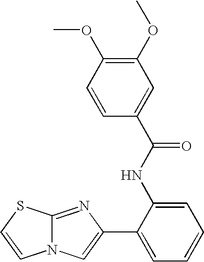 | | A′ | B |
| 204 | 410 | 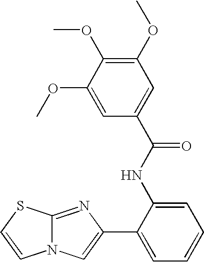 | | A′ | A |
| 205 | 437 | 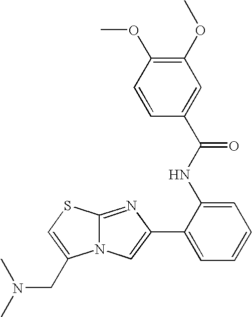 | | A | A |
| 206 | 467 | 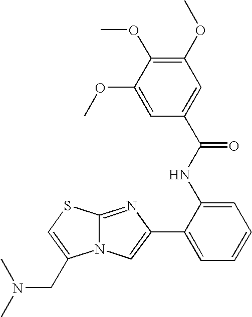 | | A | A |
| 207 | 478 | 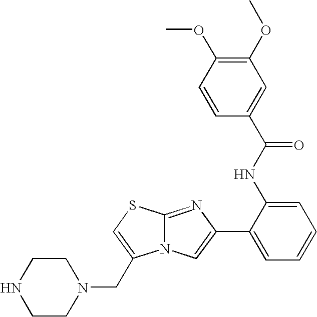 | | A | A |
| 208 | 508 | 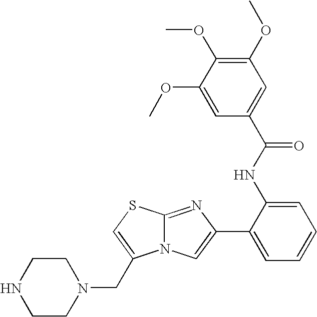 | | A | A |
| 209 | 479 | 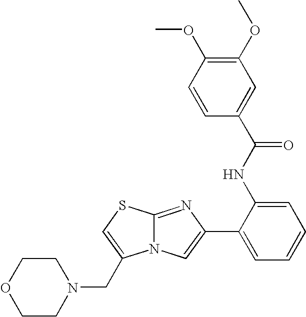 | | A | A |
| 210 | 509 | 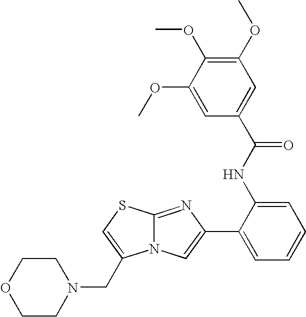 | | A | B |
| 211 | | 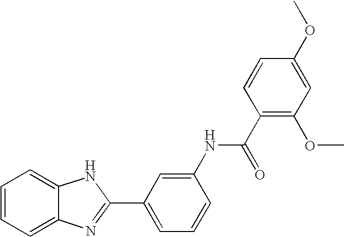 | | A′ | B |
| 212 | 362 | 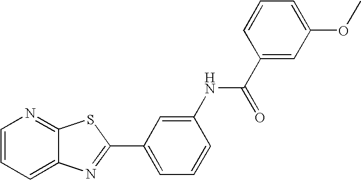 | | A | B |
| 213 | 392 | 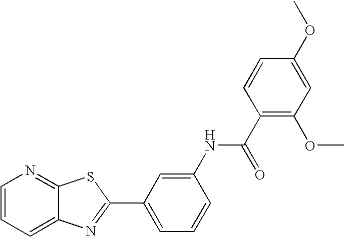 | | A′ | B |
| 214 | 392 | 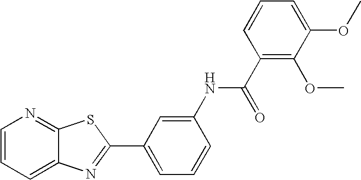 | | A | B |
| 215 | 392 | 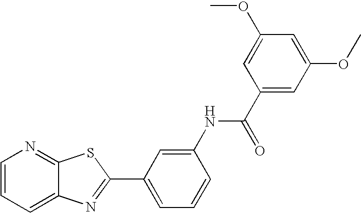 | | A′ | B |
| 216 | 362 | 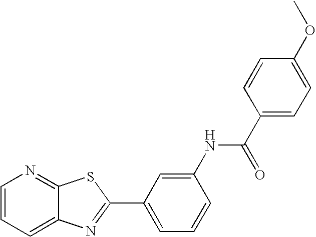 | | A′ | B |
| 217 | 422 | 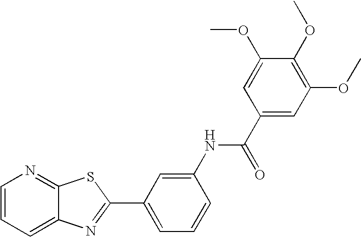 | | A′ | A |
| 218 | 405 | 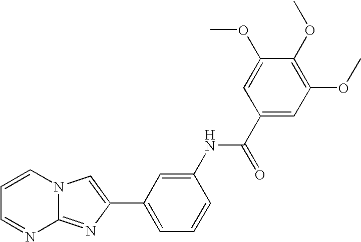 | | A′ | A |
| 219 | 419 | 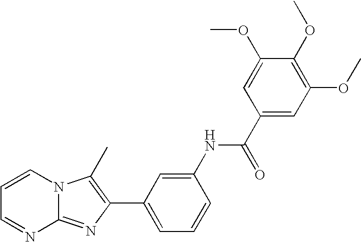 | | A | A |
| 220 | 423 | 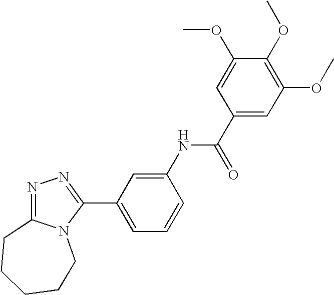 | | NA | C |
| 221 | 321.4 | 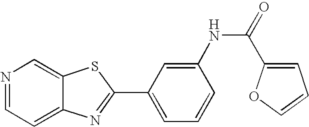 | | A | B |
| 222 | 366.8 | 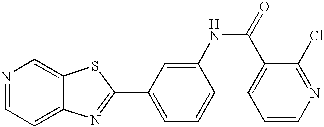 | | A | B |
| 223 | 337.4 | 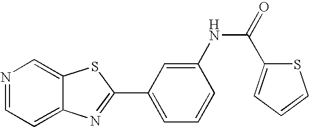 | | A′ | B |
| 225 | 332.4 | 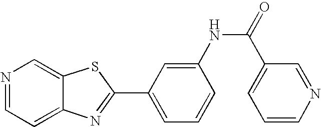 | | NA | C |
| 226 | 412.5 | 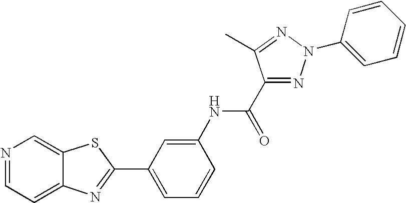 | | | |
| 227 | 333.4 | 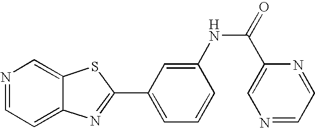 | | NA | C |
| 228 | 391.5 | 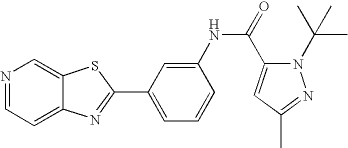 | | A | A |
| 229 | 418.5 | 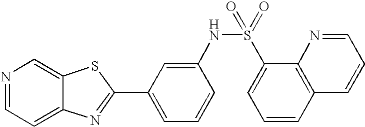 | | NA | C |
| 230 | 459.5 | 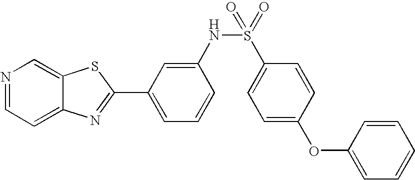 | | NA | C |
| 231 | 425.5 | 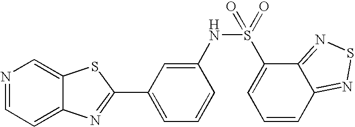 | | NA | C |
| 232 | 423.5 | 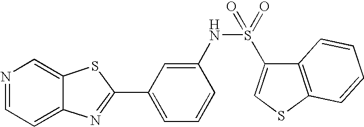 | | NA | C |
| 234 | 449.5 | 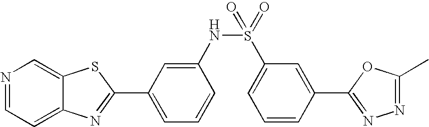 | | NA | C |
| 235 | 436.3 | 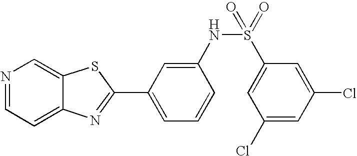 | | NA | C |
| 236 | 423.5 | 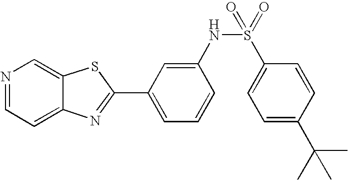 | | NA | C |
| 237 | 450.5 | 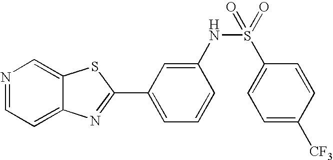 | | NA | C |
| 238 | 480.5 | 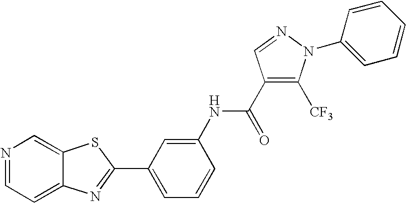 | | A′ | B |
| 239 | 466.5 | 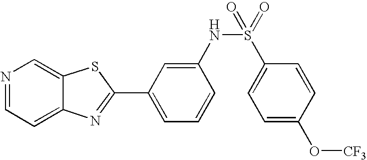 | | A | B |
| 240 | 392.4 | 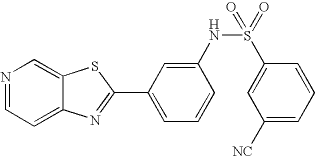 | | NA | C |
| 241 | 397.2 | 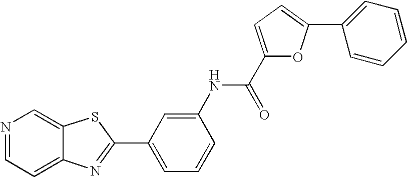 | | A′ | B |
| 244 | 332.4 | 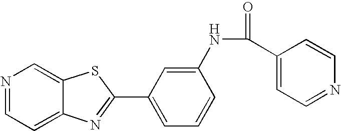 | | NA | C |
| 245 | 423.5 | 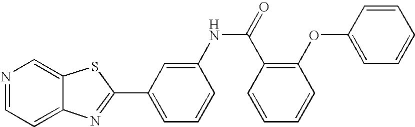 | | A′ | B |
| 246 | 391.5 | 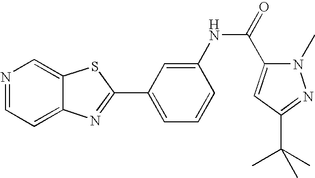 | | A′ | B |
| 247 | 413.5 | 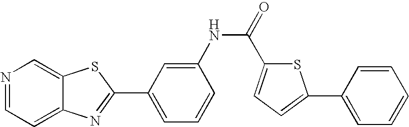 | | NA | C |
| 248 | 467.4 | 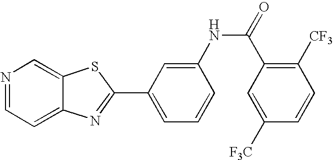 | | A | B |
| 249 | 395.9 | 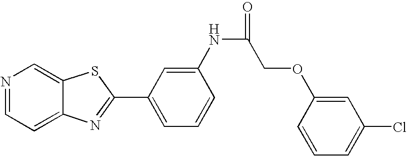 | | NA | C |
| 250 | 385.5 | 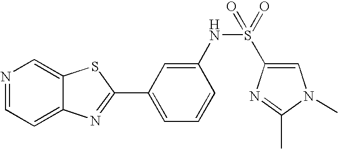 | | NA | C |
| 251 | 425.5 | 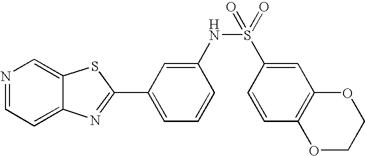 | | NA | C |
| 252 | 453.5 | 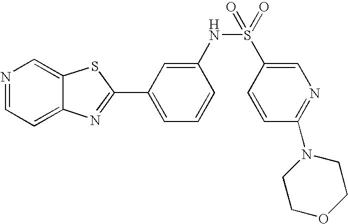 | | NA | C |
| 253 | 373.1 | 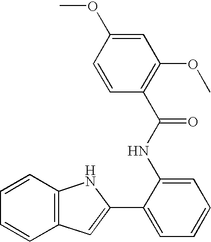 | | NA | C |
| 254 | 373 | 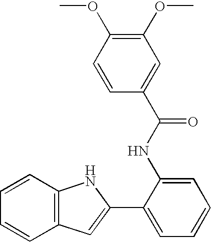 | | A′ | B |
| 255 | 403 | 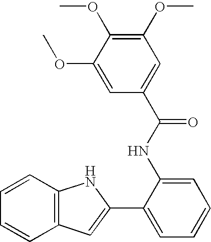 | | A′ | B |
| 256 | 356 | 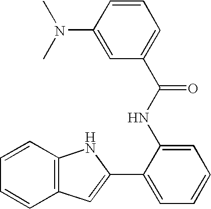 | | NA | C |
| 257 | 413.1 | 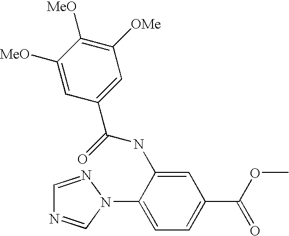 | | NA | C |
| 258 | 383.1 | 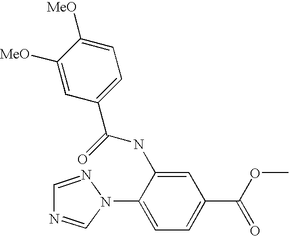 | | A | B |
| 259 | 405.1 | 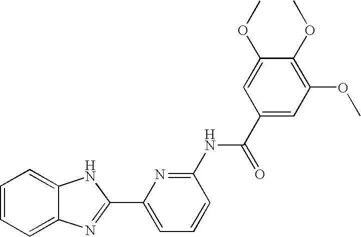 | | A′ | A |
| 260 | 375.1 | 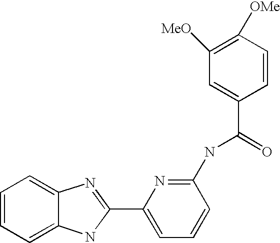 | | A′ | A |
| 261 | 375.1 | 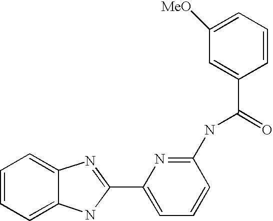 | | A′ | A |
| 262 | 374.1 | 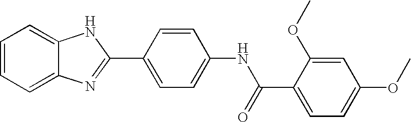 | | A′ | B |
| 263 | 404 | 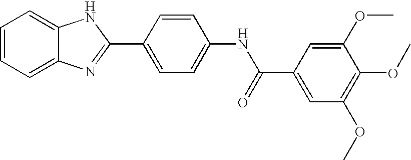 | | A | B |
| 264 | 357 | 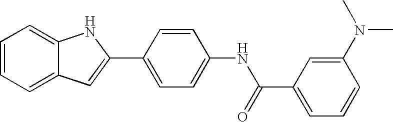 | | A′ | B |
| 265 | 374.1 | 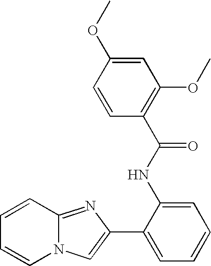 | | A | B |
| 266 | 374 | 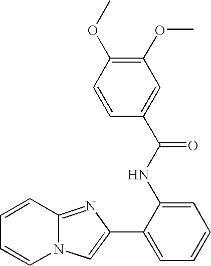 | | A′ | A |
| 267 | 404 | 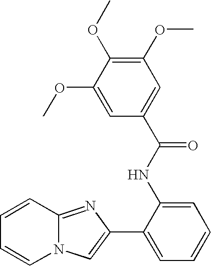 | | A′ | B |
| 268 | 357 | 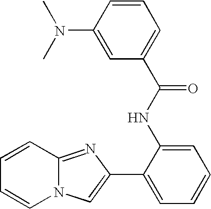 | | A′ | B |
| 270 | 478 | 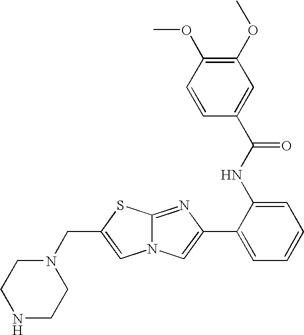 | | A | A |
| 271 | 508 | 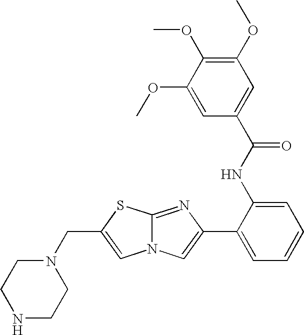 | | A′ | A |
| 272 | 392 | 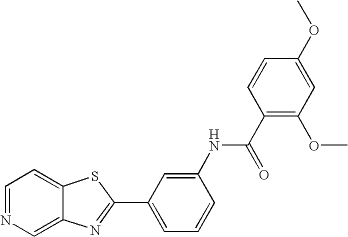 | | A′ | B |
| 273 | 422 | 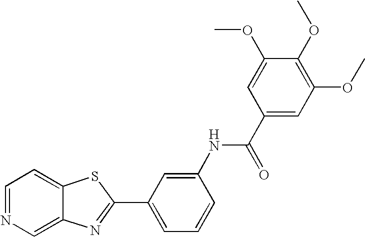 | | A | A |
| 276 | 375.1 | 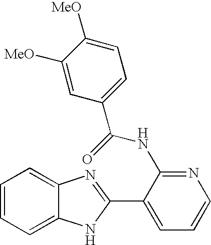 | | A′ | B |
| 280 | 381.5 | 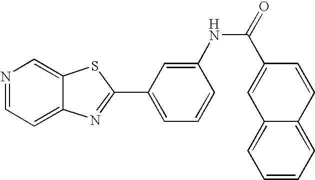 | | | |
| 282 | 386.5 | 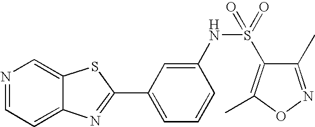 | | NA | C |
| 283 | 451.6 | 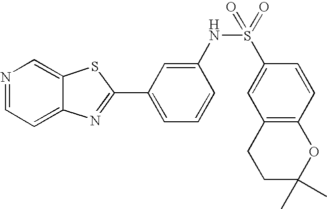 | | NA | C |
| 284 | 439.5 | 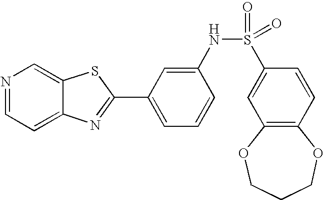 | | NA | C |
| 285 | 440.5 | 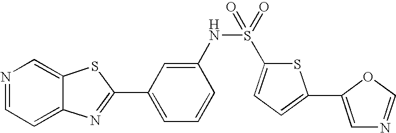 | | NA | C |
| 286 | 390.1 | 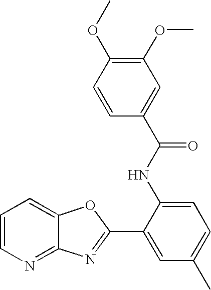 | | NA | C |
| 287 | 457.6 | 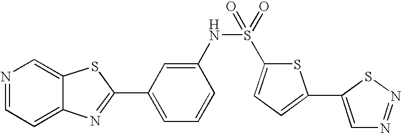 | | | |
| 288 | 424.5 | 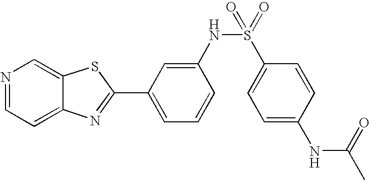 | | A | B |
| 289 | 427.5 | 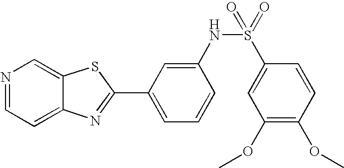 | | A | B |
| 290 | 445.5 | 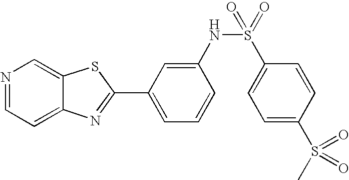 | | NA | C |
| 292 | 420.1 | 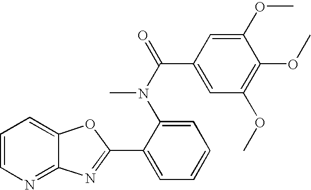 | | D | |
| 293 | 404.1 | 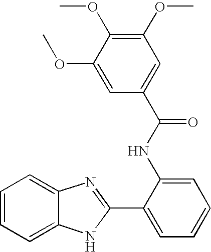 | | A′ | A |
| 294 | 374 | 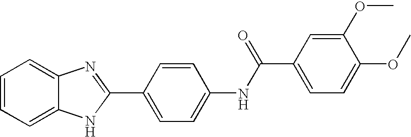 | | A′ | B |
| 295 | 252.1 | 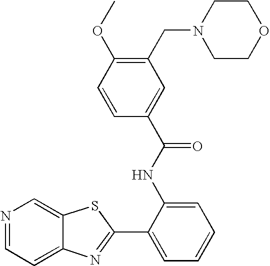 | | A′ | B |
| 296 | 376 | 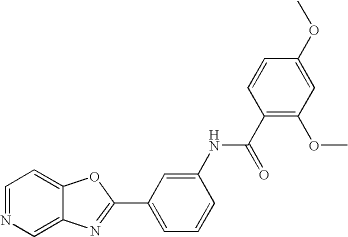 | | A | B |
| 297 | 376 | 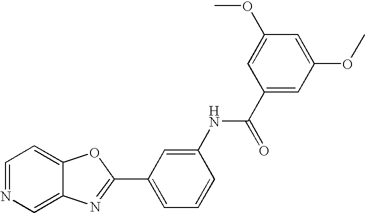 | | A | B |
| 298 | 406 | 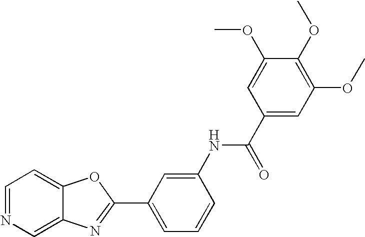 | | A | B |
| 299 | 359 | 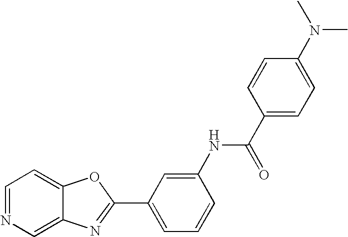 | | A | B |
| 303 | 437.5 | 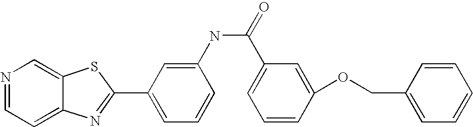 | | NA | |
| 304 | 336.4 | 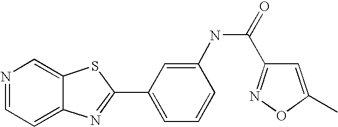 | | A′ | B |
| 305 | 414.5 | 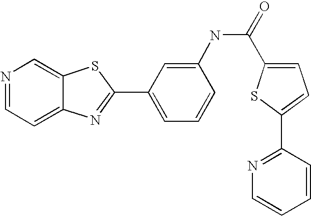 | | NA | |
| 306 | 424.5 | 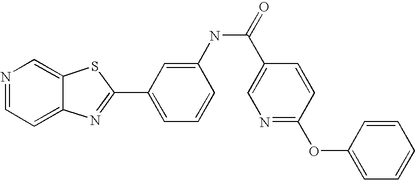 | | A | B |
| 307 | 382.4 | 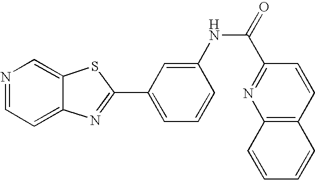 | | A′ | A |
| 308 | 400.3 | 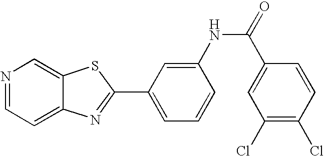 | | NA | |
| 309 | 367.4 | 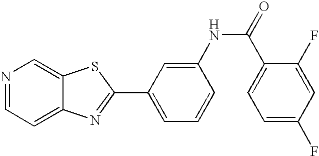 | | NA | |
| 310 | 387.5 | 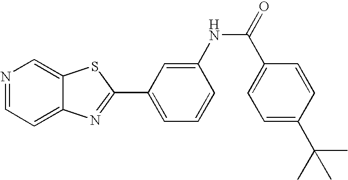 | | NA | |
| 311 | 359 | 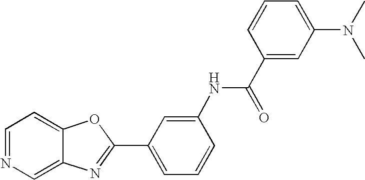 | | A | B |
| 313 | 418.1 | 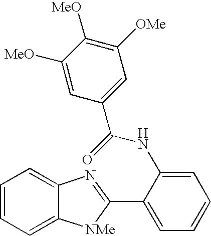 | | A | B |
| 314 | 388.1 | 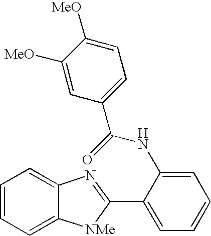 | | A | B |
| 315 | 388.1 | 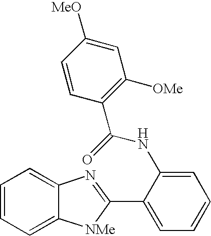 | | NA | |
| 317 | 392 | 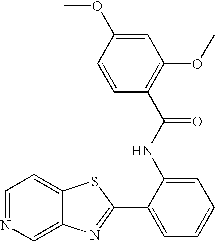 | | A′ | B |
| 318 | 392 | 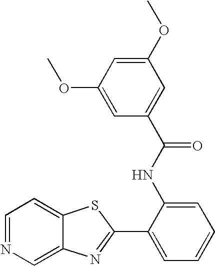 | | A | B |
| 319 | 422 | 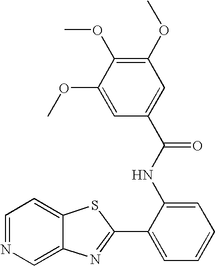 | | A′ | B |
| 320 | 375 | 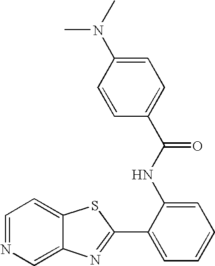 | | A′ | B |
| 321 | 375 | 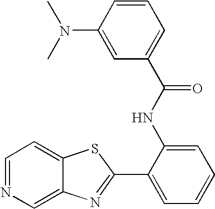 | | A′ | B |
| 322 | 392 | 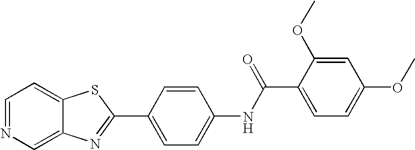 | | NA | |
| 323 | 392 | 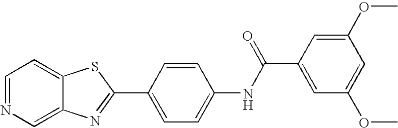 | | A | B |
| 324 | 422 | 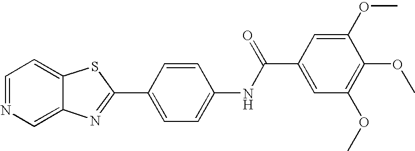 | | NA | |
| 325 | 375 | 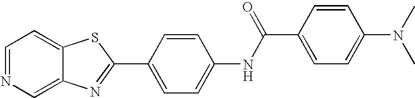 | | NA | |
| 326 | 478 | 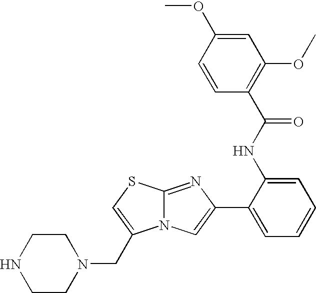 | | A | B |
| 327 | 461 | 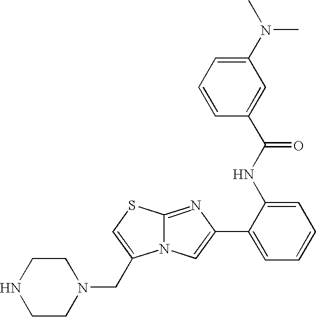 | | A | A |
| 328 | 418 | 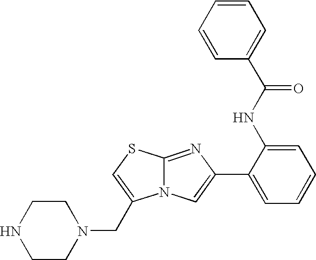 | | B | A |
| 329 | 462 | 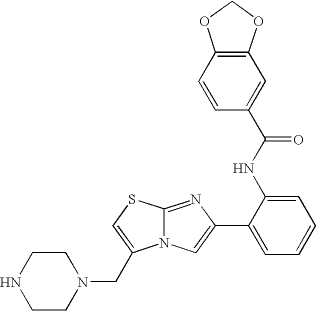 | | A | A |
| 330 | 443 | 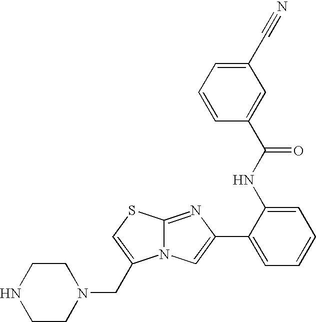 | | A | A |
| 331 | 335 | 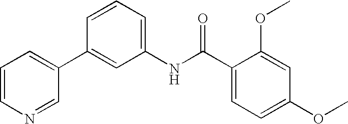 | | A | B |
| 332 | 335.1 | 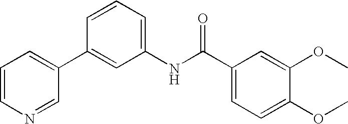 | | A | B |
| 333 | 365 | 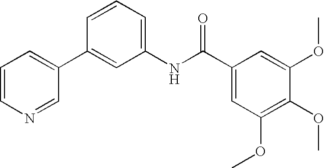 | | A | B |
| 334 | 340.1 | 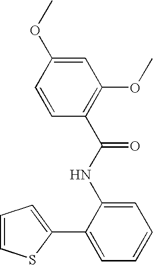 | | A | B |
| 335 | 340 | 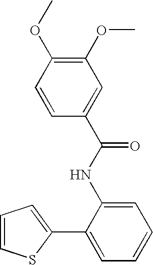 | | A | B |
| 336 10 | 370 | 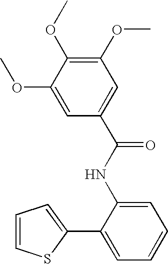 | | A | B |
| 337 | 443 | 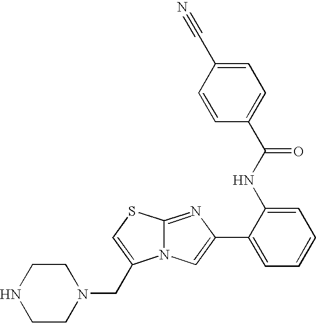 | | NA | |
| 338 | 458 | 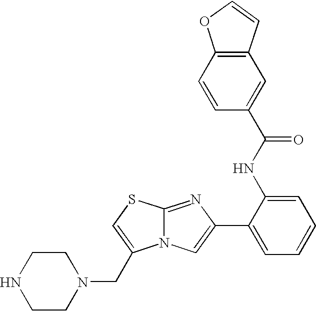 | | A | A |
| 339 | 376 | 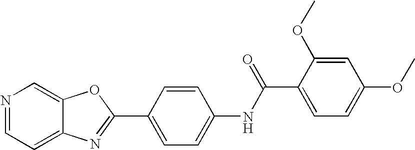 | | NA | |
| 340 | 376 | 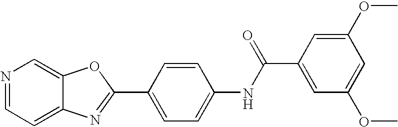 | | NA | |
| 341 | 406 | 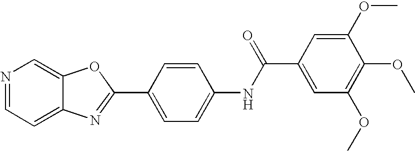 | | A′ | B |
| 342 | 359 | 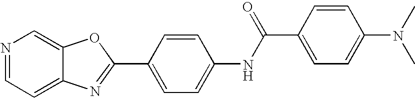 | | NA | |
| 343 | 406 | 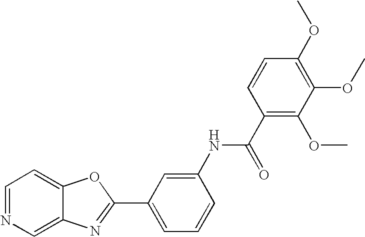 | | A | B |
| 344 | 375 | 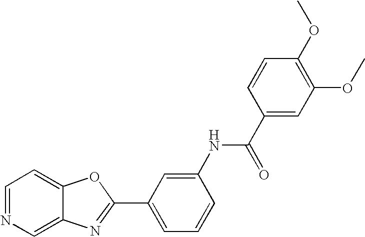 | | A′ | B |
| 345 | 376 | 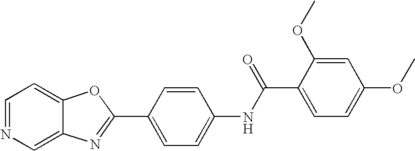 | | NA | |
| 346 | 376 | 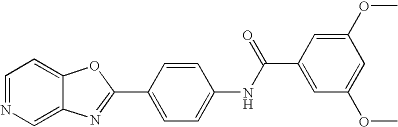 | | NA | |
| 347 | 406 | 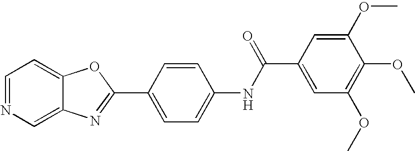 | | A′ | B |
| 348 | 359 | 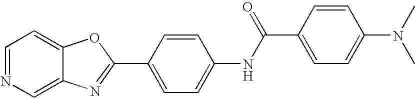 | | NA | |
| 349 | 375 | 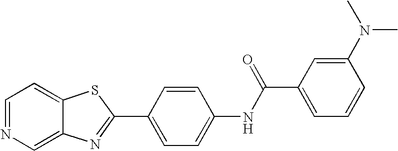 | | NA | |
| 350 | 431.1 | 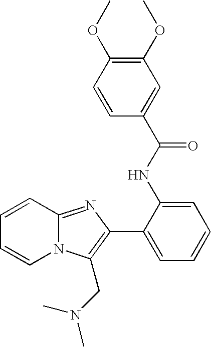 | | B | B |
| 351 | 461 | 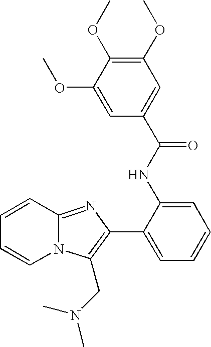 | | A | B |
| 359 | 472.1 | 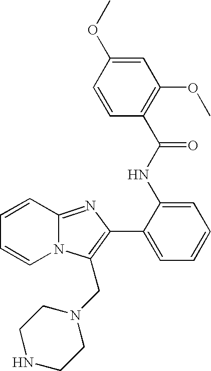 | | NA | |
| 362 | 502 | 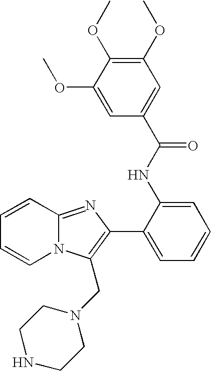 | | B | B |
| 364 | 472 | 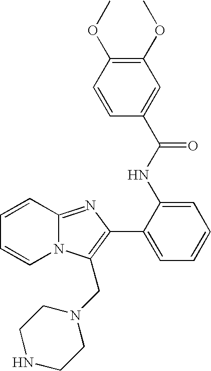 | | D | A |
| 367 | 359.1 | 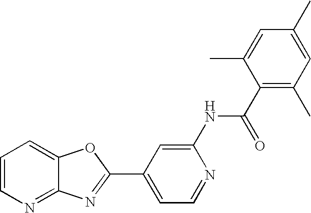 | | NA | |
| 369 | 350.8 | 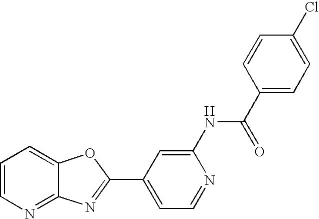 | | NA | |
| 370 | 437.0 | 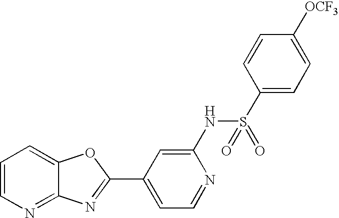 | | NA | |
| 371 | 381.1 | 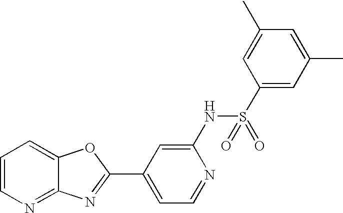 | | NA | |
| 372 | 431.1 | 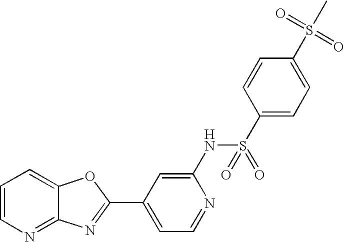 | | NA | |
| 373 | 445.0 | 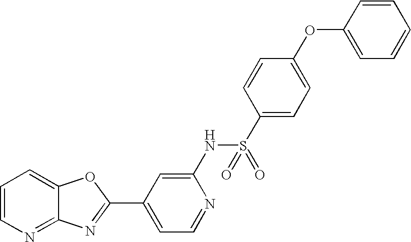 | | NA | |
| 374 | 421.1 | 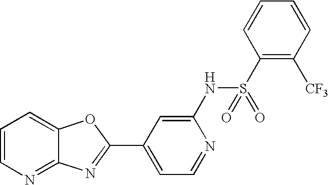 | | NA | |
| 375 | 358.2 | 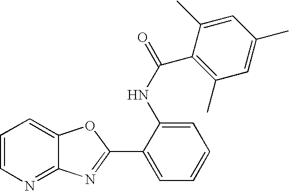 | | NA | |
| 376 | 350.0 | 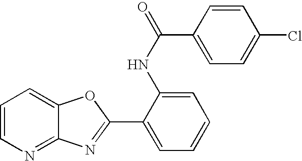 | | NA | |
| 377 | | 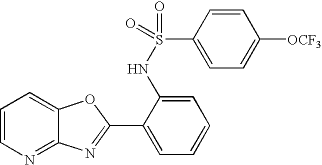 | | NA | |
| 378 | 412.1 | 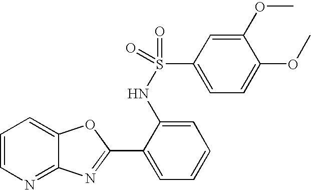 | | NA | |
| 379 | 380.0 | 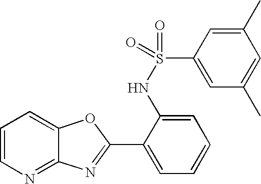 | | NA | |
| 380 | 429.9 | 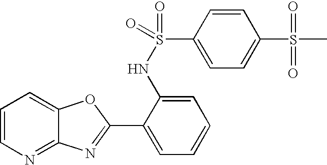 | | NA | |
| 381 | 444.1 | 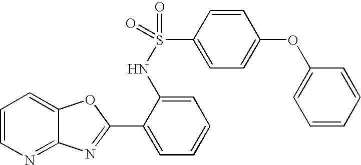 | | NA | |
| 382 | 420.0 | 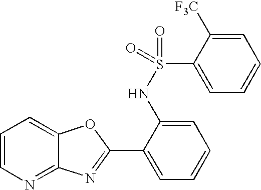 | | NA | |
| 383 | 515.7 | 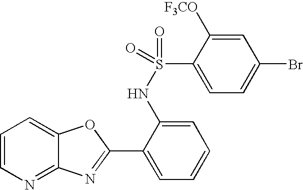 | | NA | |
| 384 | 487.8 | 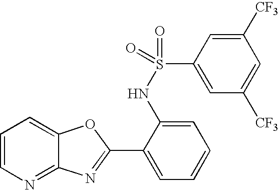 | | NA | |
| 385 | 397.2 | 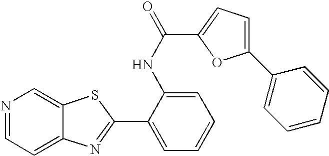 | | NA | |
| 387 | 366.8 | 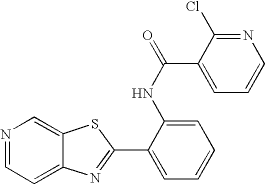 | | NA | |
| 390 | 412.5 | 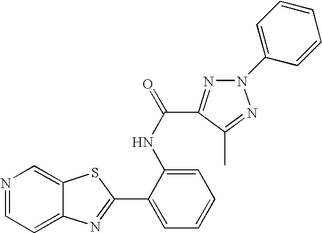 | | A | B |
| 391 | 333.4 | 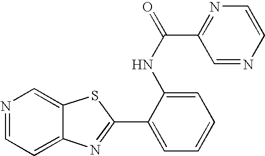 | | A | B |
| 392 | 424.5 | 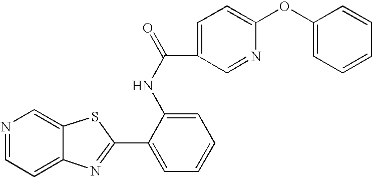 | | A | B |
| 393 | 400.3 | 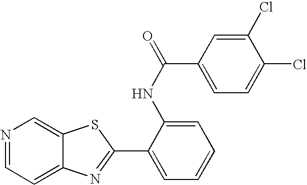 | | NA | |
| 394 | 457.6 | 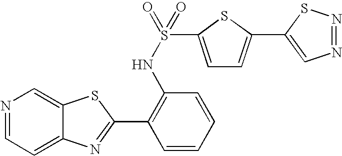 | | NA | |
| 396 | 389.5 | 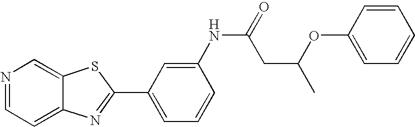 | | A | B |
| 398 | 460.1 | 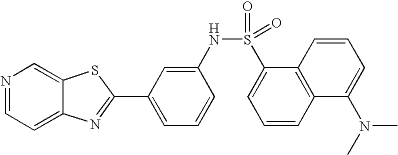 | | A | B |
| 399 | 424.5 | 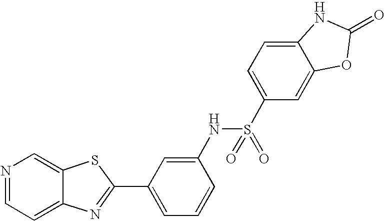 | | A | B |
| 400 | 392 | 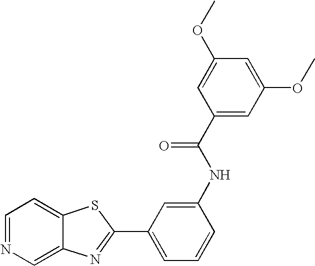 | | NA | |
| 401 | 422 | 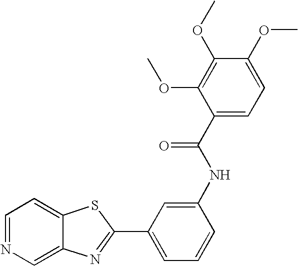 | | A | B |
| 402 | 375 | 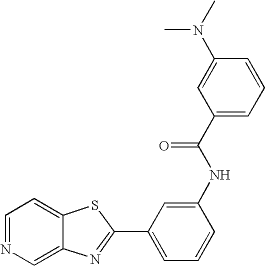 | | A′ | B |
| 403 | 375 | 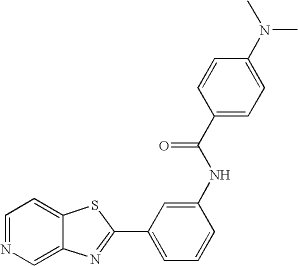 | | A′ | B |
| 404 | 376 | 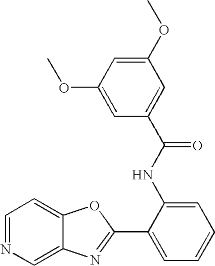 | | A′ | B |
| 405 | 406 | 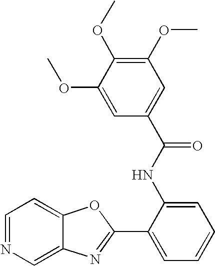 | | A′ | B |
| 406 | 359 | 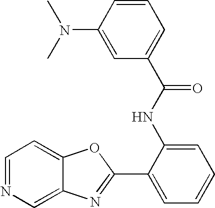 | | A′ | B |
| 407 | 359 | 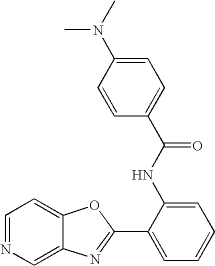 | | A′ | B |
| 408 | 359 | 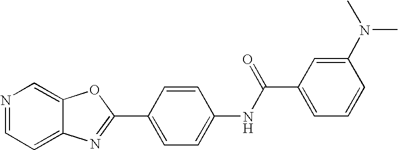 | | B | B |
| 409 | 422 | 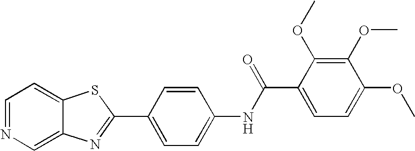 | | NA | |
| 410 | 359 | 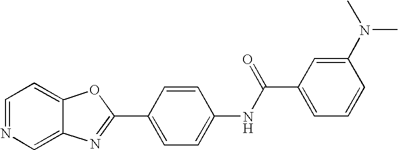 | | NA | |
| 411 | 422 | 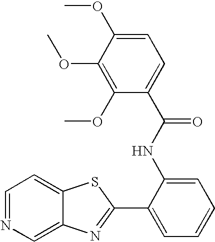 | | A | B |
| 412 | 406 | 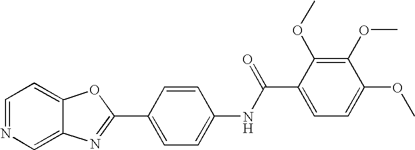 | | NA | |
| 413 | 385.1 | 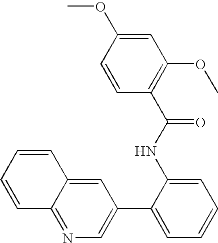 | | B | B |
| 414 | 385 | 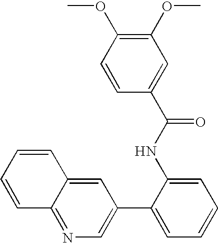 | | A | B |
| 415 | 415 | 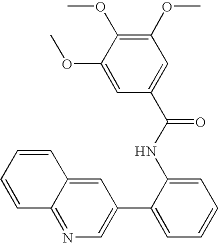 | | B | B |
| 416 | 368 | 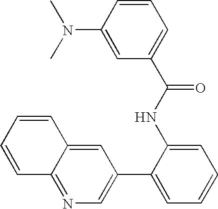 | | NA | |
| 419 | 406 | 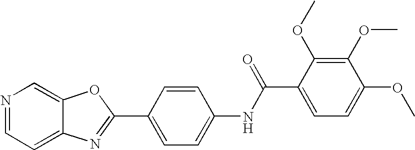 | | NA | |
| 420 | 376 | 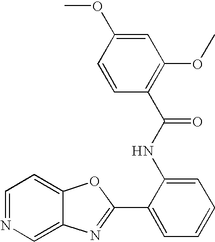 | | NA | |
| 421 | 406 | 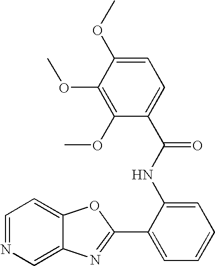 | | A | B |
| 422 | 382 | 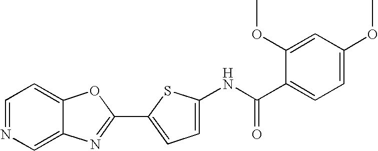 | | A | B |
| 423 | 382 | 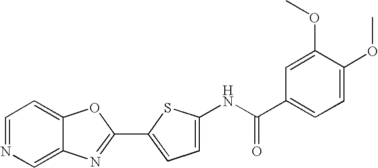 | | A′ | B |
| 424 | 382 | 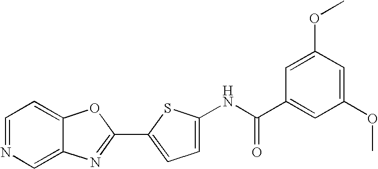 | | NA | |
| 425 | 412 | 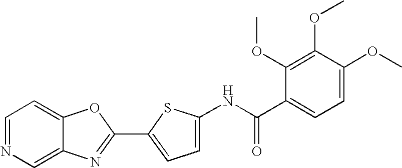 | | NA | |
| 426 | 412 | 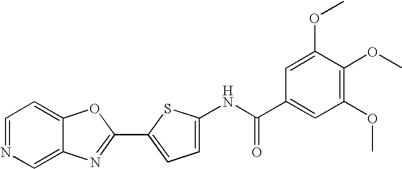 | | A′ | B |
| 427 | 365 | 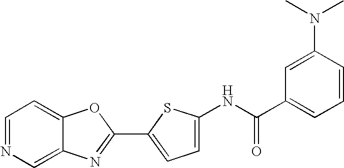 | | A′ | B |
| 428 | 365 | 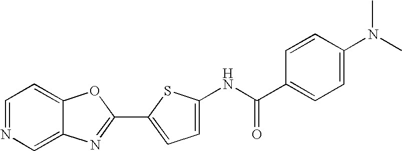 | | NA | |
| 429 | 376 | 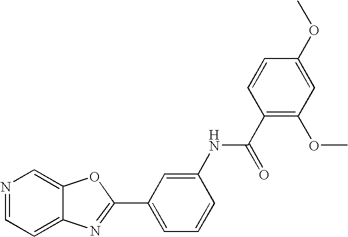 | | A′ | B |
| 430 | 406 | 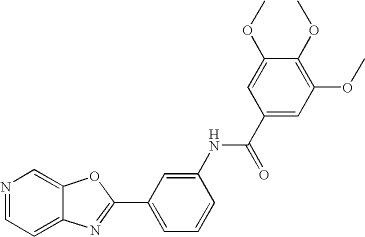 | | A | A |
| 431 | 359 | 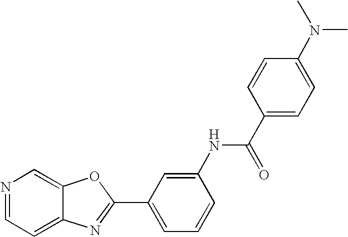 | | A | B |
| 436 | 445.1 | 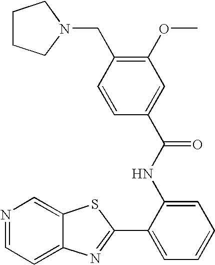 | | A | A |
| 437 | 375.1 | 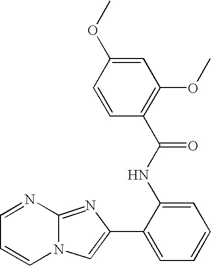 | | A | B |
| 438 | 375 | 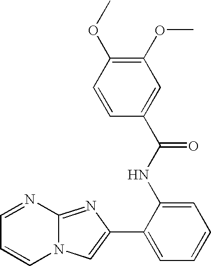 | | A′ | A |
| 439 | 405 | 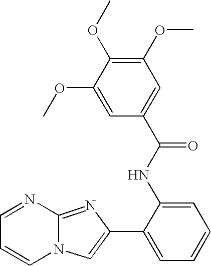 | | A′ | B |
| 440 | 468 | 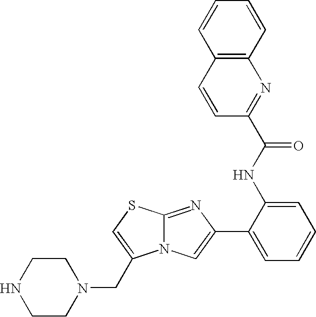 | | A′ | A |
| 441 | 470 | 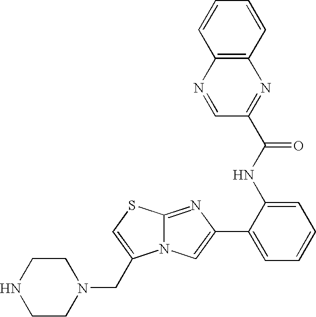 | | A′ | A |
| 442 | 472 | 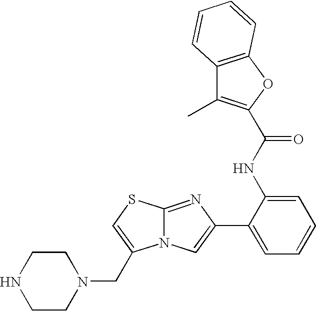 | | A′ | A |
| 443 | 436 | 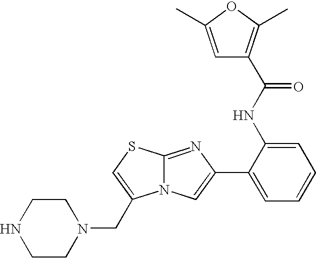 | | A | A |
| 444 | 464 | 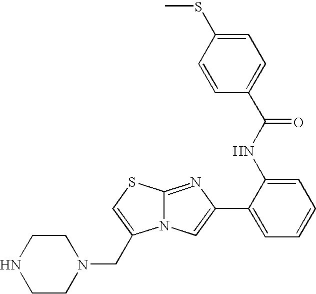 | | A | A |
| 445 | 432 | 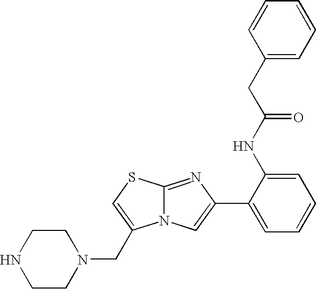 | | A | B |
| 446 | 424 | 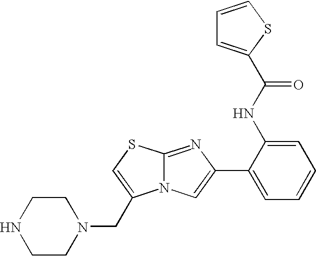 | | A | B |
| 447 | 484 | 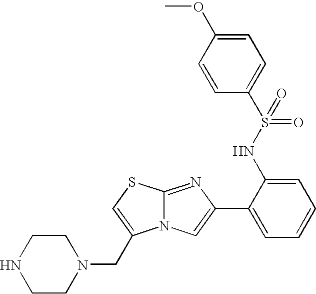 | | NA | |
| 448 | 510 | 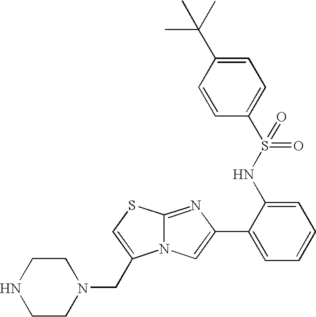 | | NA | |
| 449 | 376 | 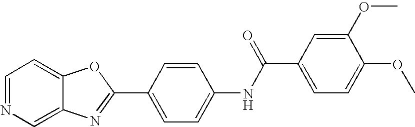 | | NA | |
| 450 | 392 | 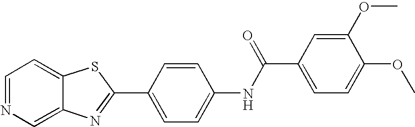 | | NA | |
| 451 | 376 | 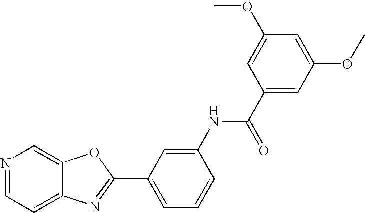 | | A | B |
| 452 | 406 | 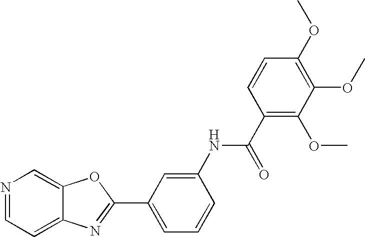 | | A | B |
| 453 | 359 | 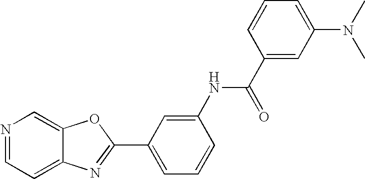 | | A | B |
| 454 | 376 | 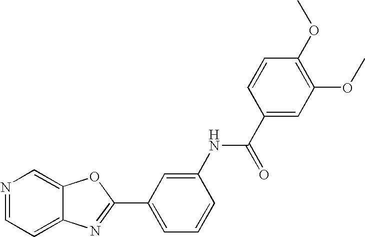 | | A | B |
| 455 | 376 | 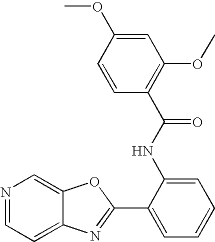 | | A′ | B |
| 456 | 376 | 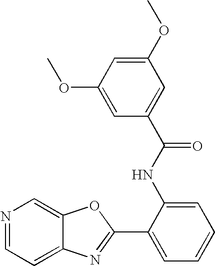 | | A′ | B |
| 457 | 406 | 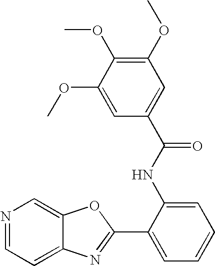 | | A | B |
| 458 | 359 | 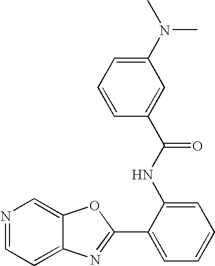 | | A′ | B |
| 459 | 359 | 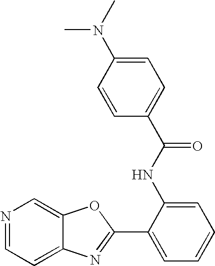 | | A′ | B |
| 460 | 359.4 | 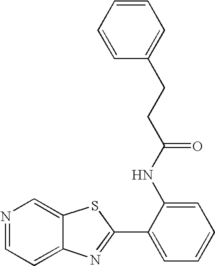 | | A | B |
| 461 | 367.4 | 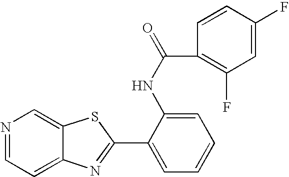 | | NA | |
| 462 | 391.5 | 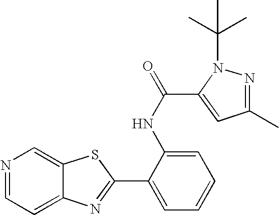 | | A′ | B |
| 463 | 375.4 | 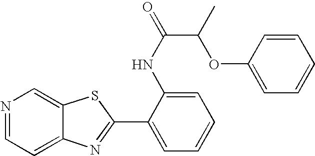 | | A | B |
| 465 | 395.9 | 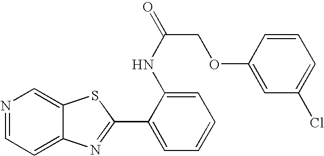 | | NA | |
| 466 | 445.5 | 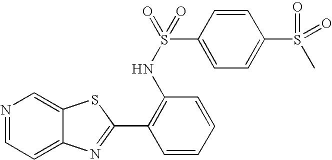 | | NA | |
| 467 | 427.2 | 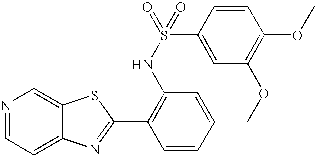 | | NA | |
| 468 | 345.0 | 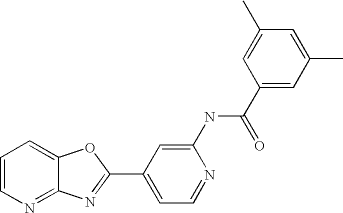 | | A | B |
| 469 | 435.4 | 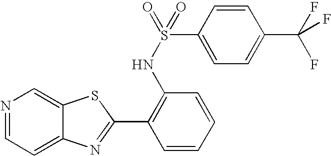 | | NA | |
| 470 | 365.0 | 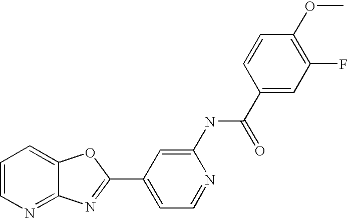 | | NA | |
| 471 | 488.9 | 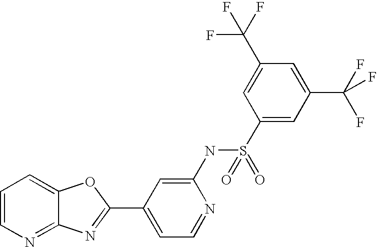 | | NA | |
| 472 | 437.5 | 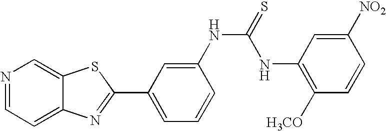 | | NA | |
| 473 | 420.5 | 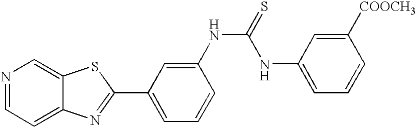 | | A′ | B |
| 474 | | 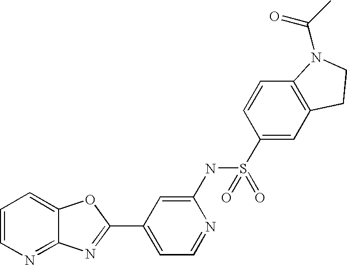 | | NA | |
| 475 | 408.6 | 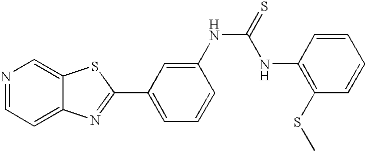 | | NA | |
| 476 | 410.9 | 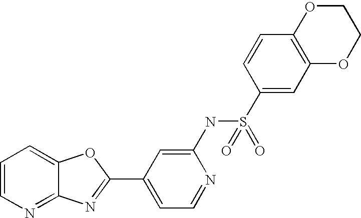 | | NA | |
| 477 | 435.9 | 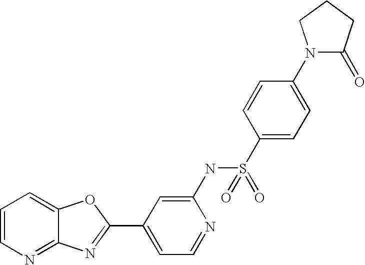 | | NA | |
| 478 | 404.8 | 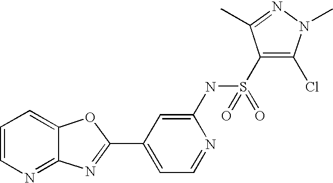 | | NA | |
| 479 | 420.9 | 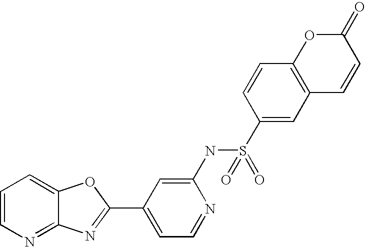 | | NA | |
| 481 | 428.5 | 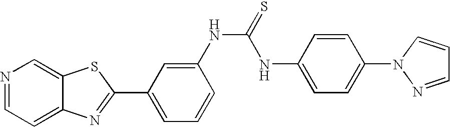 | | A | B |
| 482 | 344.1 | 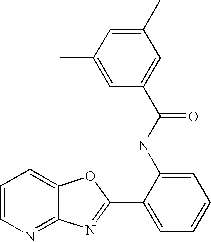 | | A′ | B |
| 483 | 364.1 | 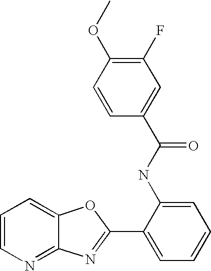 | | NA | |
| 484 | 448.6 | 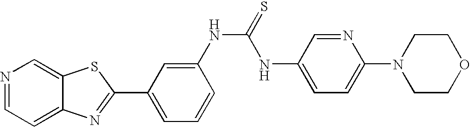 | | A | B |
| 485 | 363.5 | 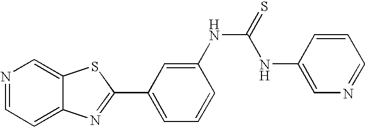 | | A | A |
| 486 | 385.9 | 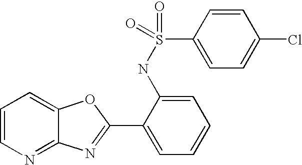 | | NA | |
| 487 | 396.1 | 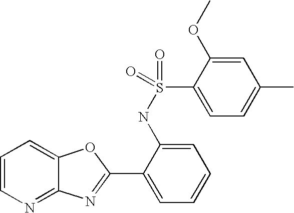 | | NA | |
| 488 | 435.1 | 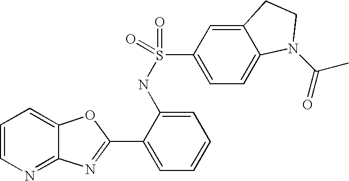 | | NA | |
| 489 | 410.1 | 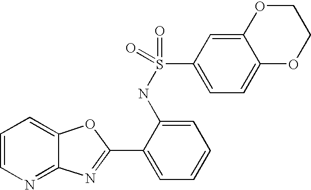 | | NA | |
| 490 | 434.9 | 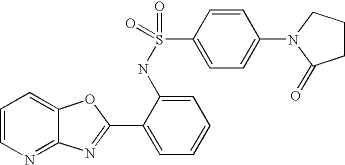 | | NA | |
| 491 | 420.5 | 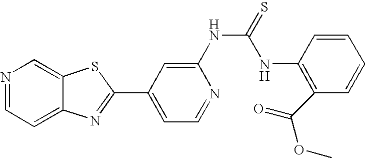 | | NA | |
| 492 | 404.0 | 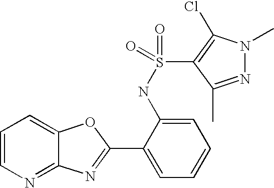 | | NA | |
| 493 | | 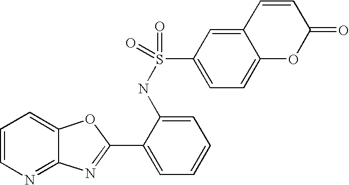 | | NA | |
| 494 | 422.9 | 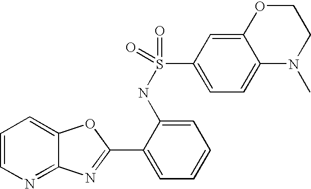 | | NA | |
| 495 | 366.0 | 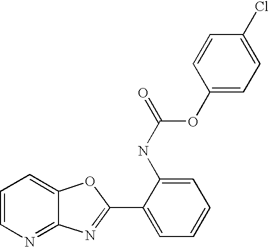 | | NA | |
| 496 | 349.9 | 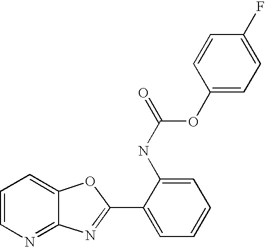 | | NA | |
| 497 | 346.0 | 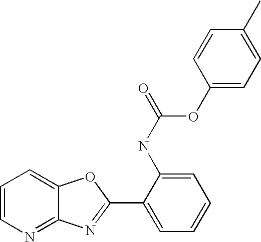 | | NA | |
| 498 | 406.5 | 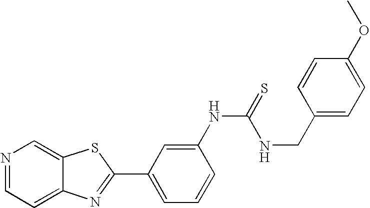 | | A | B |
| 499 | 362.1 | 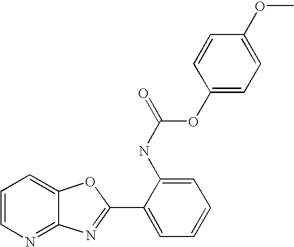 | | NA | |
| 500 | | 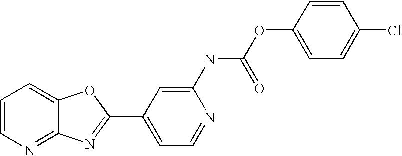 | | NA | |
| 501 | 347.1 | 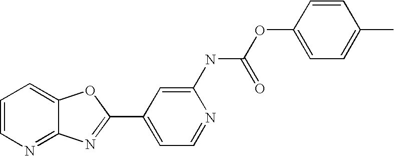 | | NA | |
| 502 | 363.1 | 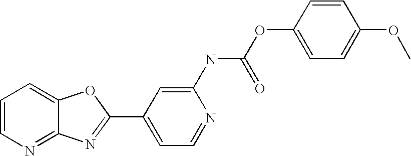 | | NA | |
| 503 | 443.1 | 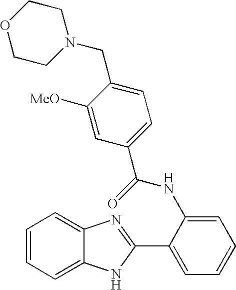 | | A′ | A |
| 504 | 358 | 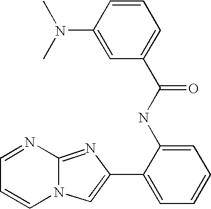 | | A | B |
| 505 | 359 | 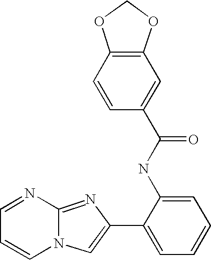 | | NA | |
| 506 | 429.1 | 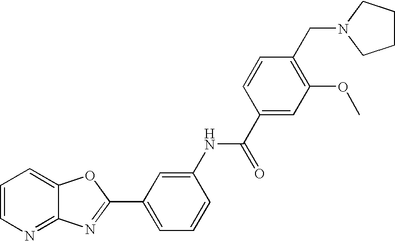 | | A | A |
| 507 | 388.1 | 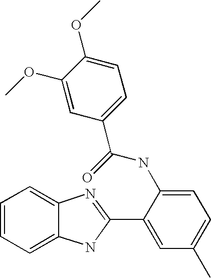 | | A′ | A |
| 508 | 366.1 | 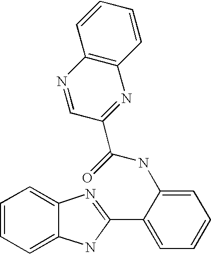 | | A′ | A |
| 510 | 457 | 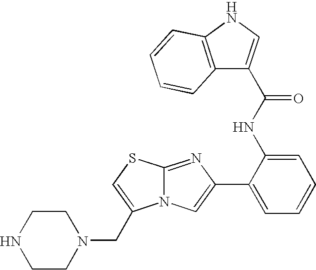 | | A′ | A |
| 511 | 460 | 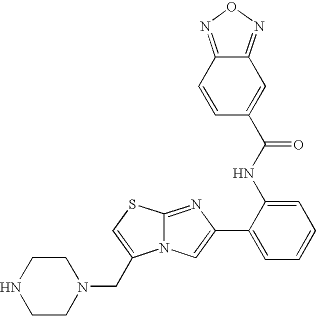 | | NA | |
| 512 | 484 | 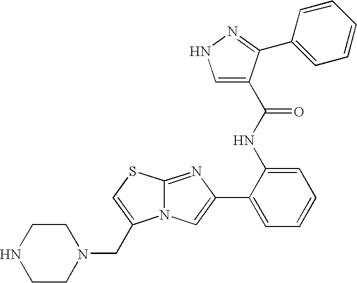 | | A′ | A |
| 513 | 470 | 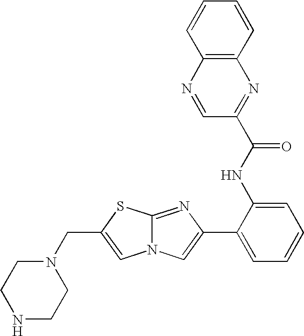 | | A′ | A |
| 514 | 466 | 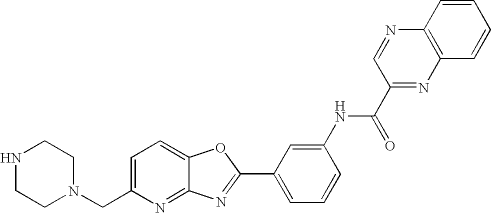 | | A | B |
| 515 | 398 | 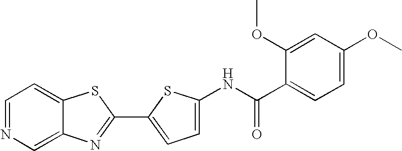 | | A′ | B |
| 516 | 398 | 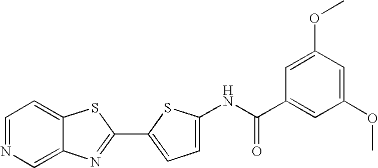 | | NA | |
| 517 | 428 | 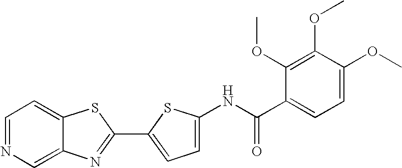 | | NA | |
| 518 | 381 | 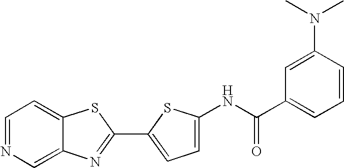 | | A′ | B |
| 519 | 381 | 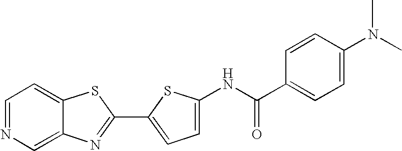 | | A′ | B |
| 520 | 428 | 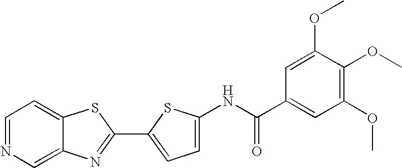 | | A′ | B |
| 521 | 375.0 | 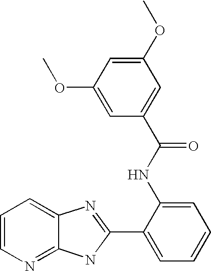 | | A′ | B |
| 522 | 405.0 | 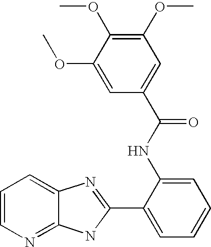 | | A | A |
| 523 | 359.0 | 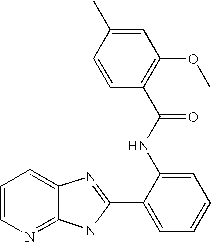 | | A | B |
| 524 | 358.0 | 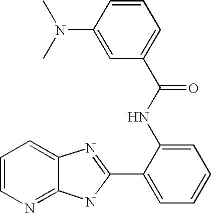 | | A′ | B |
| 525 | 375.0 | 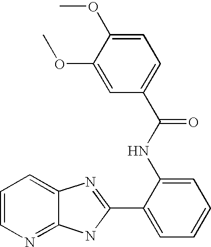 | | A′ | B |
| 526 | 400.0 | 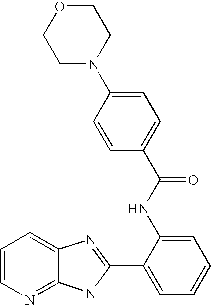 | | A′ | A |
| 527 | 398.0 | 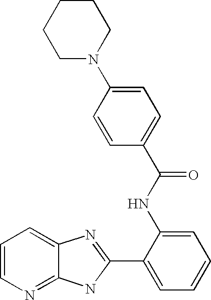 | | A′ | B |
| 528 | 412.9 | 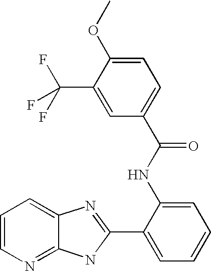 | | A′ | B |
| 529 | 399.1 | 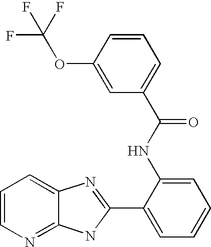 | | A′ | B |
| 530 | 359.0 | 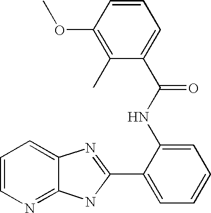 | | A′ | B |
| 531 | 345.0 | 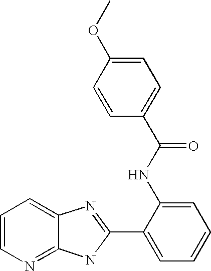 | | A′ | B |
| 532 | 345.0 | 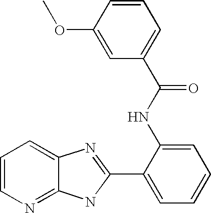 | | A′ | B |
| 533 | 315.0 | 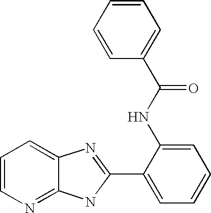 | | A′ | B |
| 534 | 358.0 | 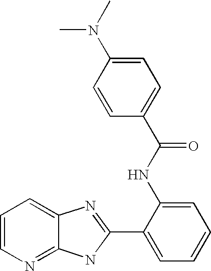 | | A′ | B |
| 535 | 428.1 | 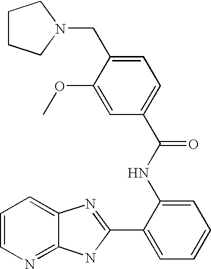 | | A | A |
| 536 | 442.1 | 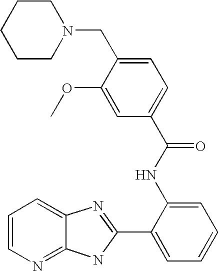 | | A | A |
| 537 | 444.0 | 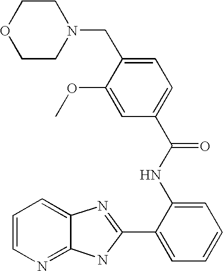 | | A′ | A |
| 538 | 443.1 | 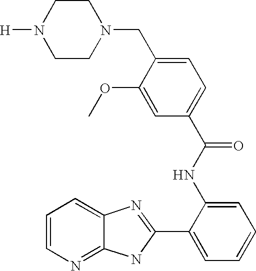 | | A | A |
| 539 | 457.1 | 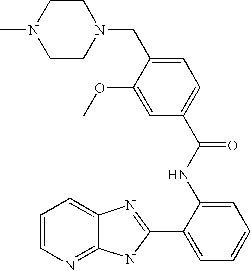 | | A′ | A |
| 540 | 402.1 | 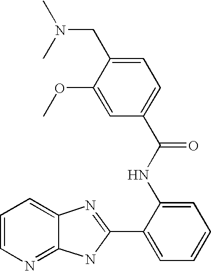 | | A | A |
| 541 | 443.1 | 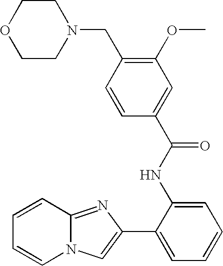 | | A | B |
| 542 | 444 | 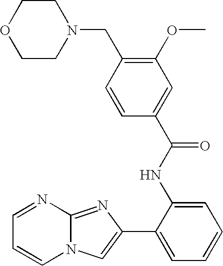 | | A | A |
| 543 | 420 | 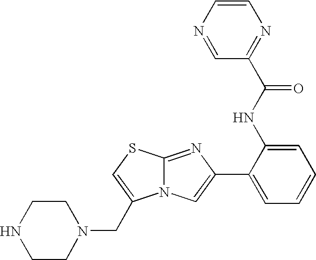 | | A′ | A |
| 544 | 474 | 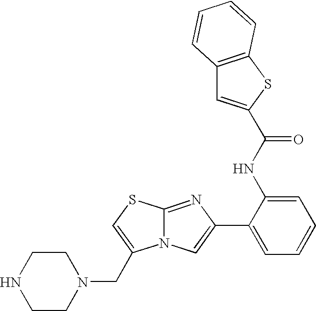 | | A | A |
| 545 | 378.1 | 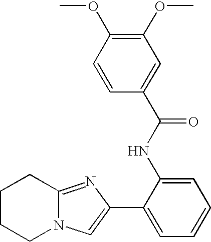 | | A | B |
| 546 | 408 | 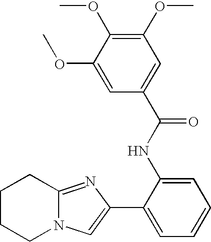 | | A | B |
| 547 | 369 | 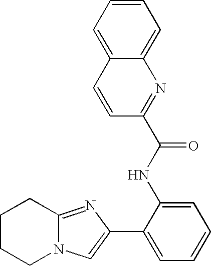 | | A′ | B |
| 548 | 370 | 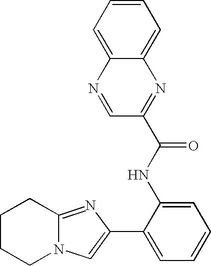 | | A′ | B |
| 556 | 365.1 | 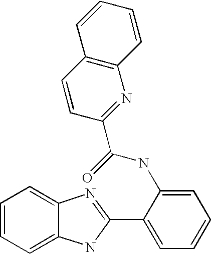 | | A′ | A |
| 557 | 542.1 | 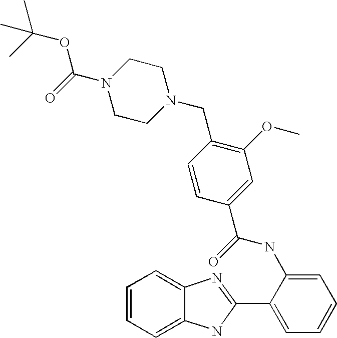 | | NA | |
| 558 | 442.1 | 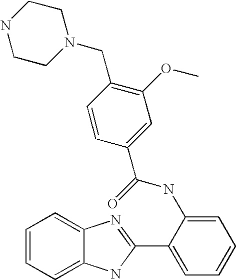 | | A′ | A |
| 559 | 508 | 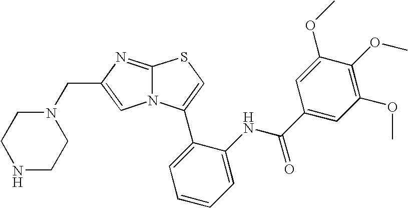 | | NA | |
| 560 | 470 | 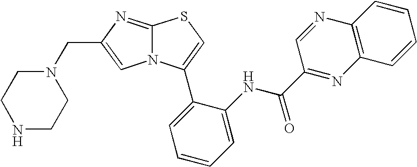 | | NA | |
| 561 | 382 | 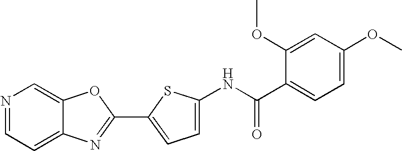 | | A′ | B |
| 562 | 382 | 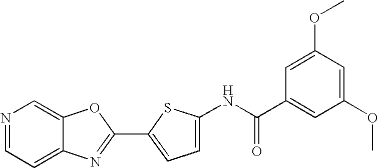 | | A′ | B |
| 563 | 412 | 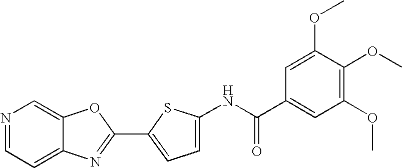 | | A | B |
| 565 | 400.8 | 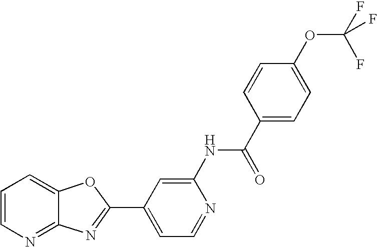 | | NA | |
| 566 | 409.9 | 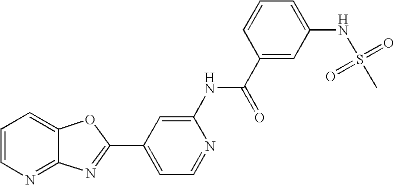 | | A | B |
| 567 | 374.9 | 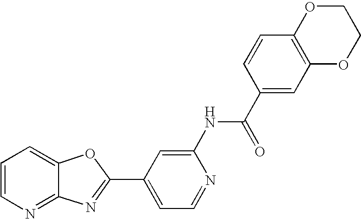 | | NA | |
| 568 | 367.0 | 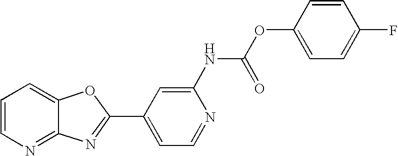 | | NA | |
| 569 | 374.9 | 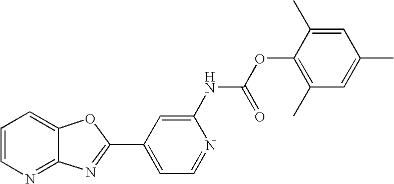 | | NA | |
| 570 | 361.0 | 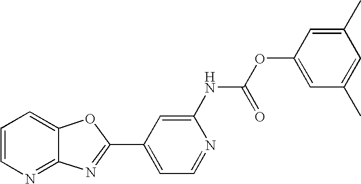 | | NA | |
| 571 | 444.0 | 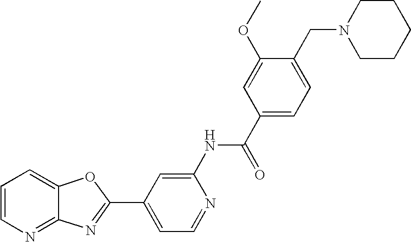 | | A | B |
| 572 | 429.9 | 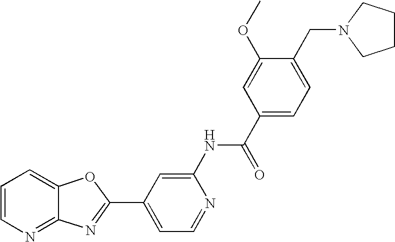 | | B | B |
| 573 | 431.8 | 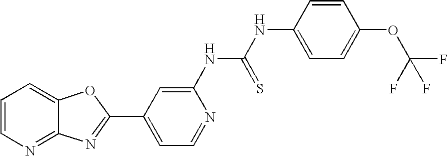 | | NA | |
| 574 | 376.2 | 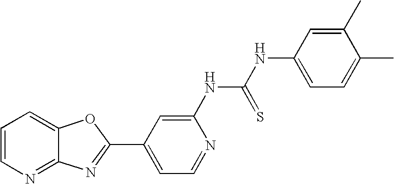 | | NA | |
| 575 | 439.9 | 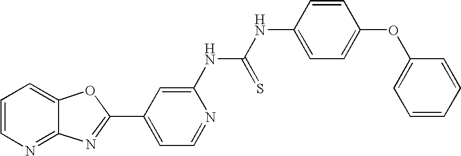 | | NA | |
| 576 | 376.1 | 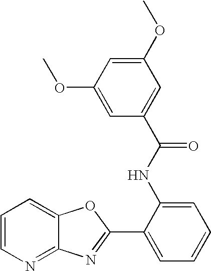 | | NA | |
| 577 | 400.1 | 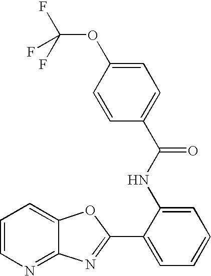 | | NA | |
| 578 | 368.1 | 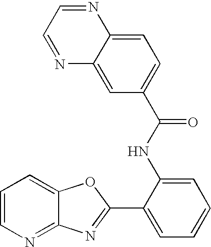 | | NA | |
| 579 | 401.1 | 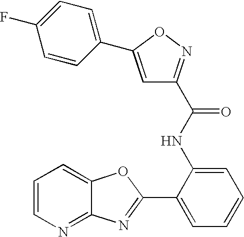 | | NA | |
| 580 | 374.0 | 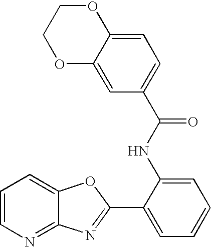 | | NA | |
| 581 | 334.0 | 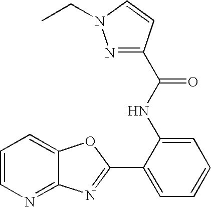 | | A | B |
| 582 | 400.0 | 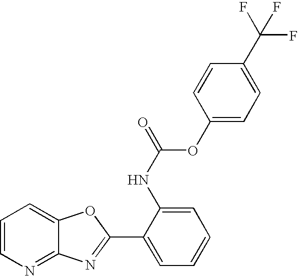 | | NA | |
| 583 | 374.1 | 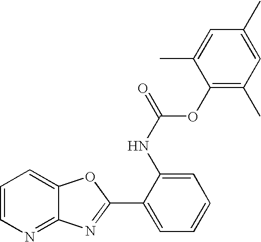 | | NA | |
| 584 | 360.0 | 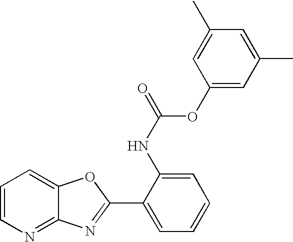 | | NA | |
| 585 | 443.1 | 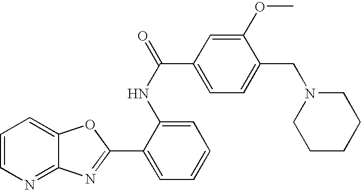 | | A | B |
| 587 | 427.1 | 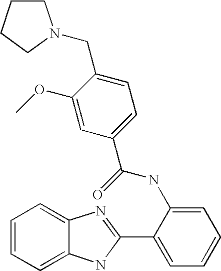 | | A′ | B |
| 588 | 520 | 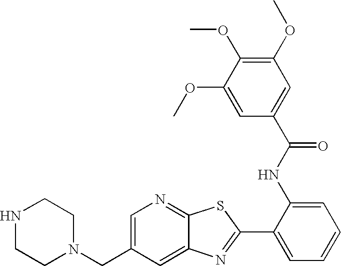 | | A | A |
| 589 | 490 | 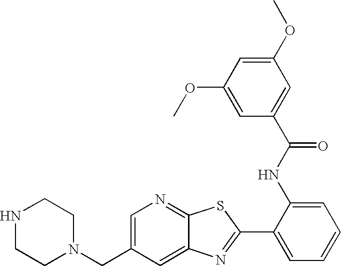 | | A′ | A |
| 590 | 474 | 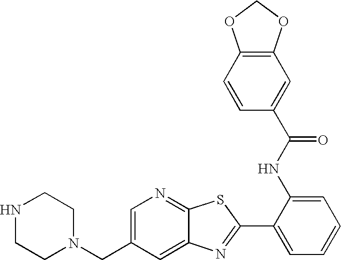 | | A | B |
| 591 | 458 | 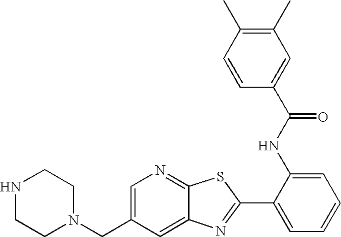 | | A′ | A |
| 592 | 455 | 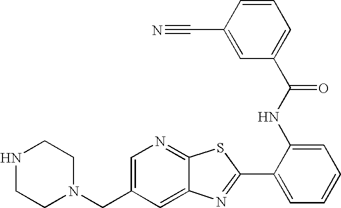 | | A′ | B |
| 593 | 473 | 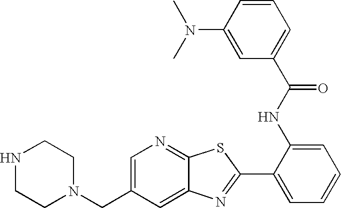 | | A′ | A |
| 594 | 466 | 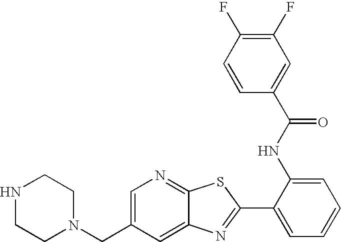 | | A | B |
| 596 | 467.4 | 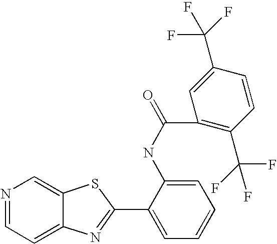 | | A′ | B |
| 597 | 377.2 | 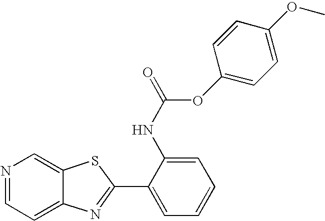 | | A′ | B |
| 599 | 422.3 | 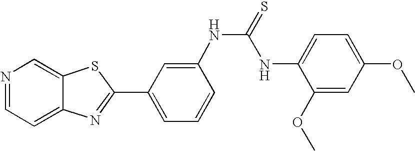 | | A′ | B |
| 600 | 422.5 | 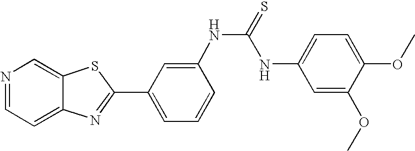 | | D | B |
| 601 | 405.5 | 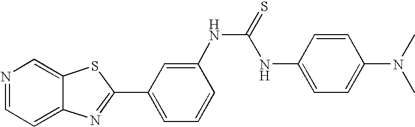 | | NA | |
| 604 | 360.4 | 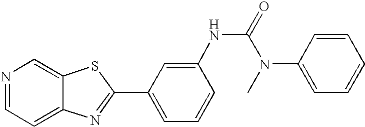 | | NA | |
| 605 | 377.4 | 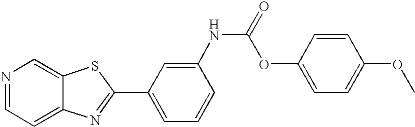 | | NA | |
| 607 | 415.4 | 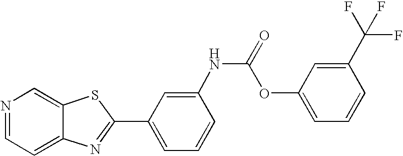 | | NA | |
| 608 | 332.4 | 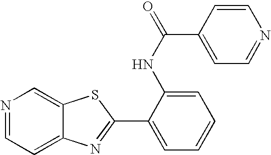 | | NA | |
| 609 | 439.3 | 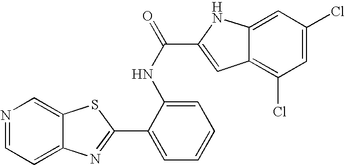 | | NA | |
| 610 | 418.6 | 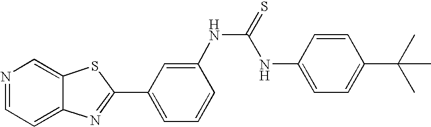 | | NA | |
| 611 | 465.6 | 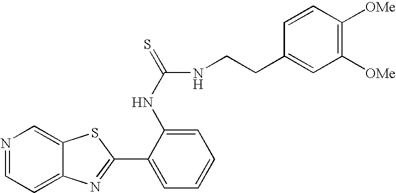 | | A′ | B |
| 612 | 402.5 | 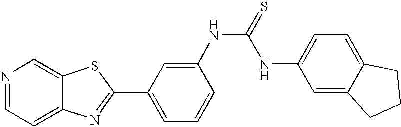 | | NA | |
| 614 | 428.5 | 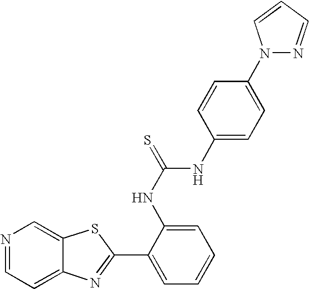 | | NA | |
| 615 | 408.6 | 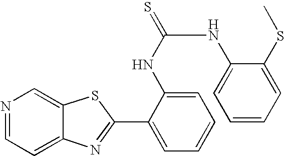 | | NA | |
| 616 | 437.5 | 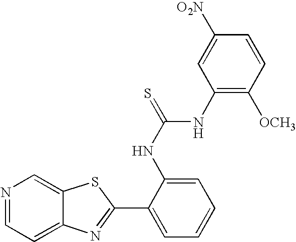 | | | |
| 617 | 471.1 | 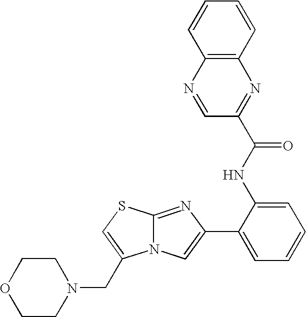 | | A′ | A |
| 618 | 469.1 | 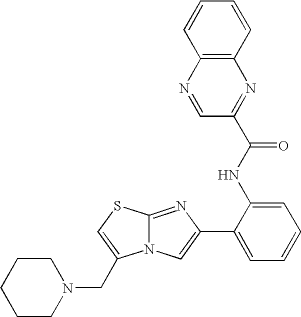 | | A′ | B |
| 619 | 455 | 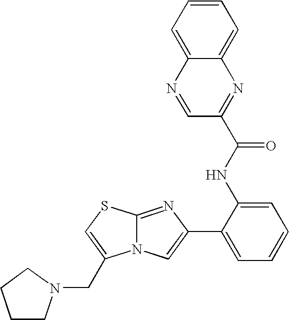 | | A′ | B |
| 620 | 493.1 | 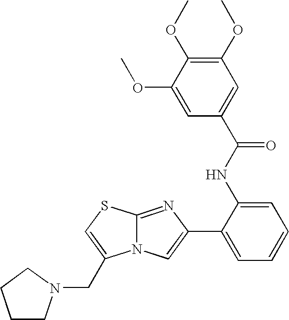 | | A′ | B |
| 621 | 472 | 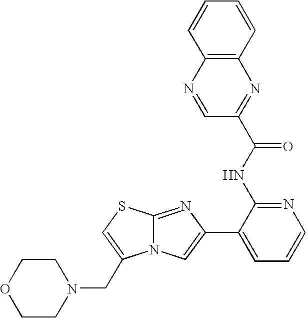 | | A′ | A |
| 622 | 482 | 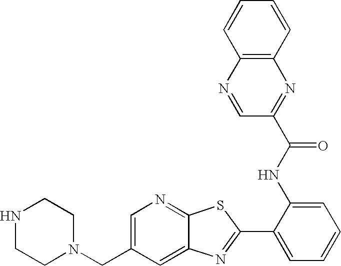 | | A′ | A |
| 623 | 437 | 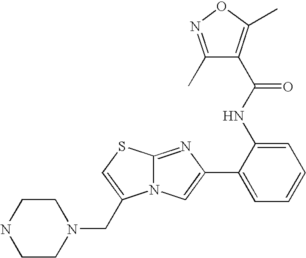 | | NA | C |
| 624 | 458 | 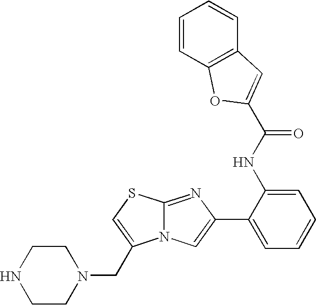 | | A′ | A |
| 625 | 496 | 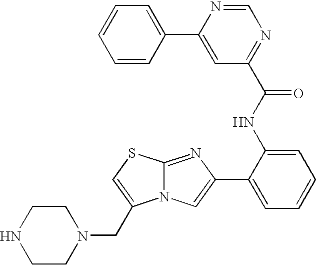 | | A′ | A |
| 628 | 574.1 | 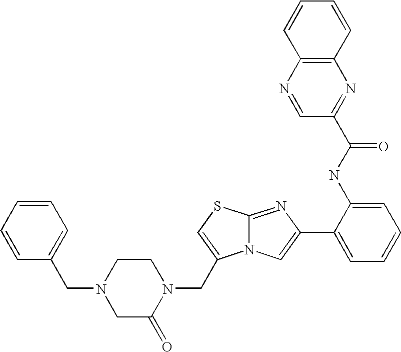 | | A′ | B |
| 629 | 429.0 | 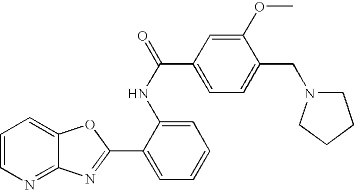 | | A | A |
| 630 | 403.1 | 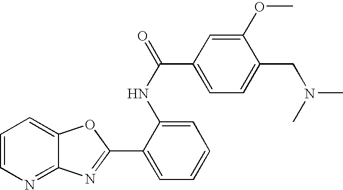 | | A | A |
| 631 | 445.0 | 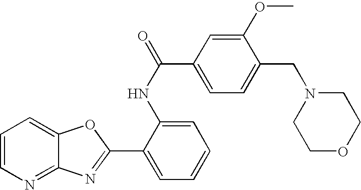 | | A′ | B |
| 632 | 458.1 | 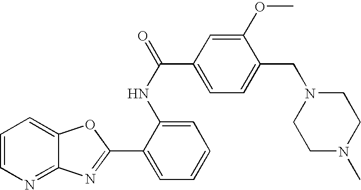 | | A | A |
| 633 | 423.3 | 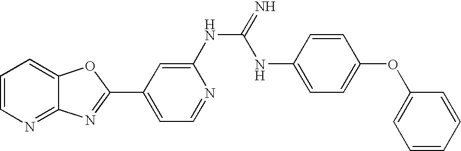 | | NA | C |
| 634 | 364.9 | 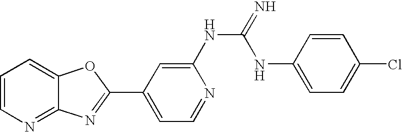 | | D | B |
| 635 | 421.1 | 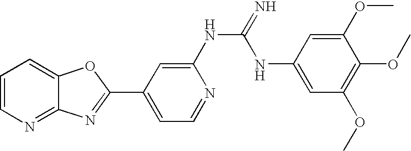 | | D | A |
| 636 | 359.9 | 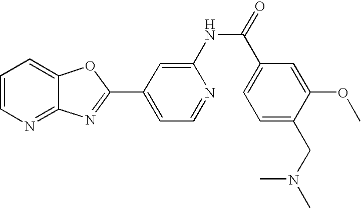 | | B | B |
| 637 | 459.3 | 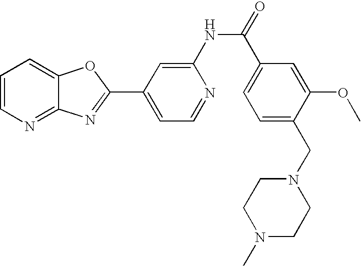 | | D | B |
| 638 | 445.2 | 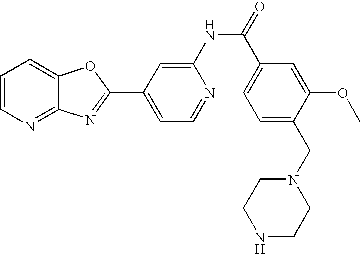 | | D | B |
| 639 | 378.9 | 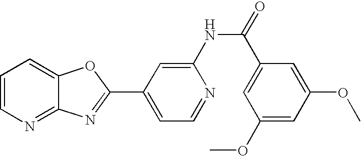 | | A | B |
| 640 | 368.9 | 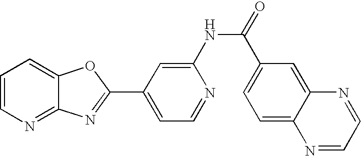 | | NA | C |
| 641 | 334.9 | 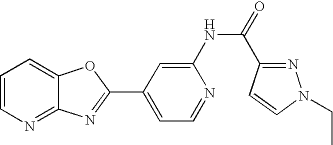 | | D | B |
| 642 | 446.2 | 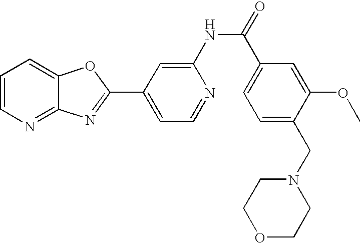 | | D | B |
| 643 | 535.1 | 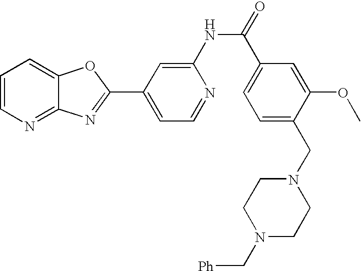 | | A | B |
| 644 | 434 | 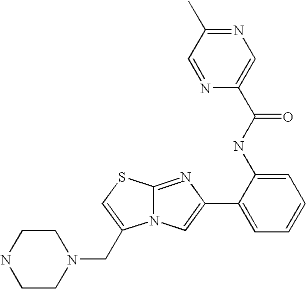 | | A | A |
| 645 | 469 | 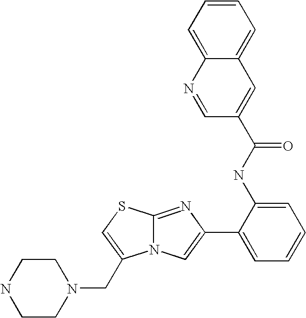 | | A′ | A |
| 646 | 481 | 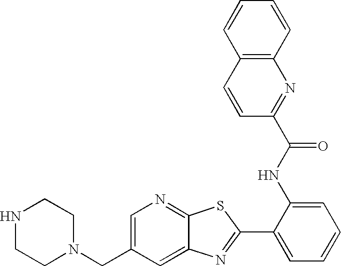 | | A | A |
| 647 | 473.1 | 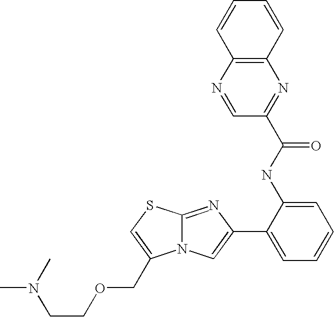 | | A′ | A |
| 648 | 402.1 | 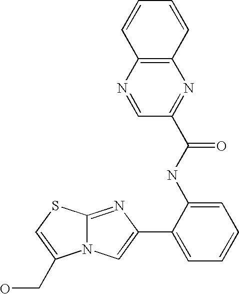 | | A′ | B |
| 649 | 512.2 | 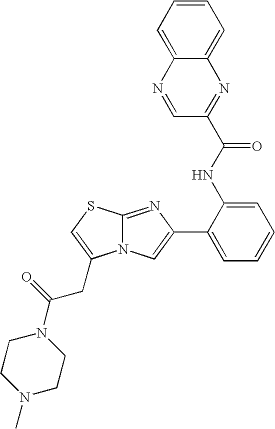 | | A′ | A |
| 650 | 570.2 | 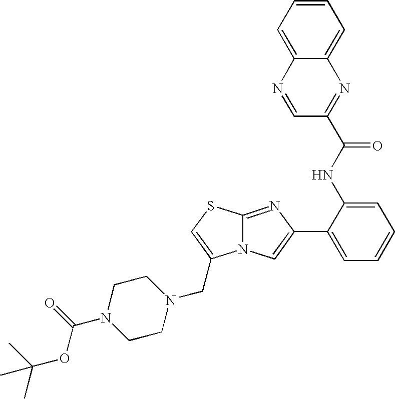 | | NA | |
| 651 | 512.2 | 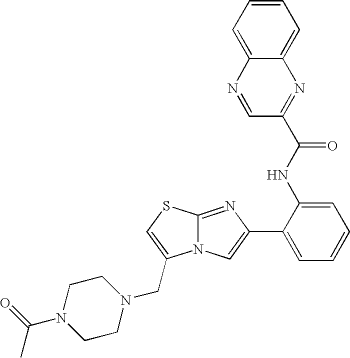 | | A′ | A |
| 655 | 393.0 | 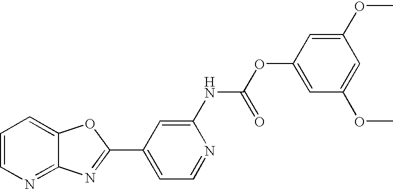 | | NA | C |
| 656 | 393.2 | 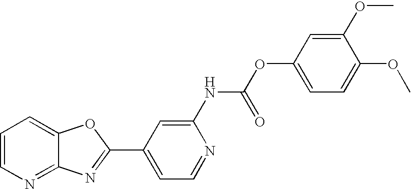 | | NA | C |
| 657 | 423.1 | 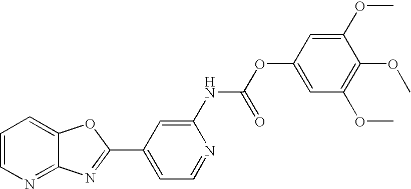 | | NA | C |
| 658 | 382.1 | 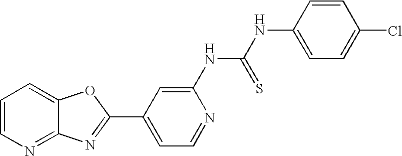 | | NA | C |
| 659 | 509.2 | 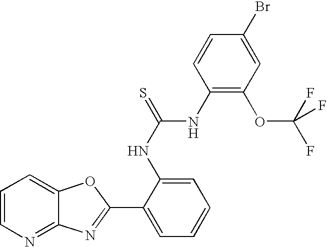 | | NA | C |
| 660 | 408.1 | 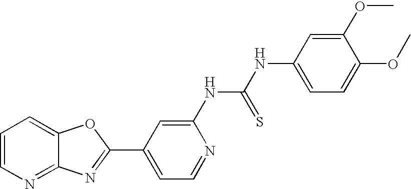 | | A | B |
| 661 | 408.2 | 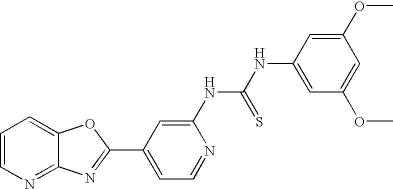 | | A′ | B |
| 662 | 378.2 | 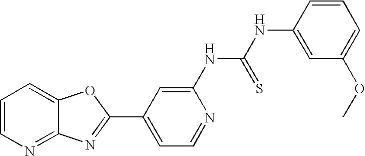 | | NA | C |
| 663 | 438.0 | 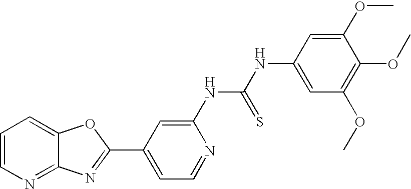 | | A | B |
| 664 | 477.8 | 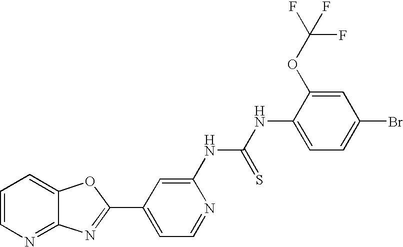 | | NA | C |
| 665 | 406.1 | 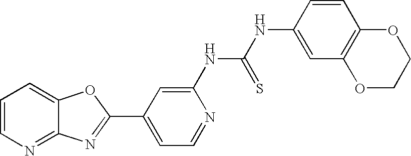 | | NA | C |
| 666 | 391.0 | 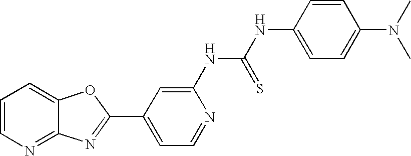 | | NA | C |
| 667 | 448.0 | 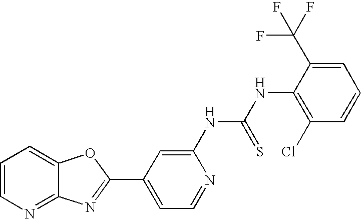 | | A | A |
| 668 | 410.0 | 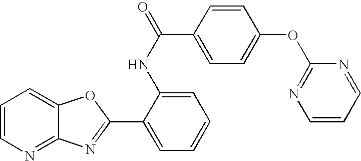 | | A | B |
| 669 | 392.0 | 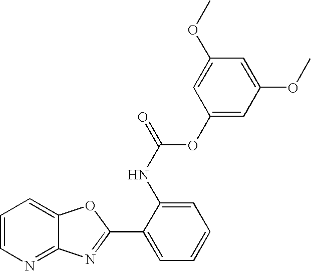 | | NA | C |
| 670 | 392.0 | 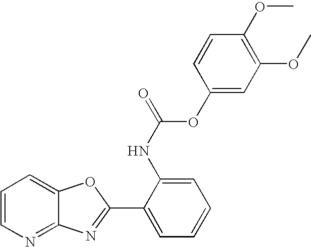 | | A | B |
| 671 | 422.0 | 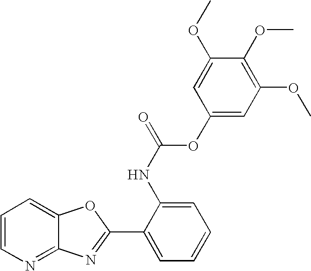 | | NA | C |
| 672 | 415.1 | 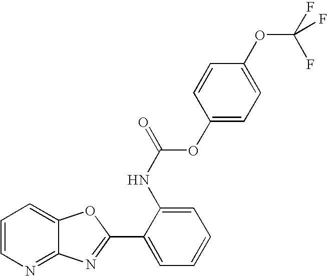 | | NA | C |
| 673 | | 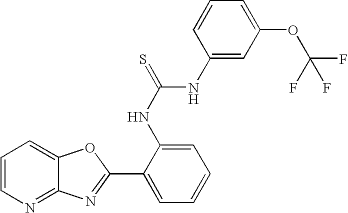 | | NA | C |
| 674 | | 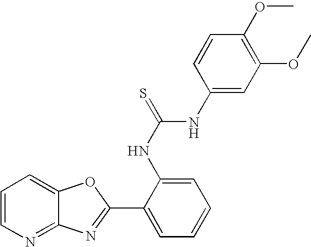 | | NA | C |
| 675 | 407.1 | 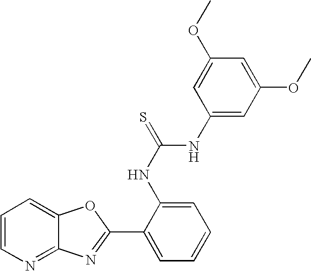 | NA | C | |
| 676 | | 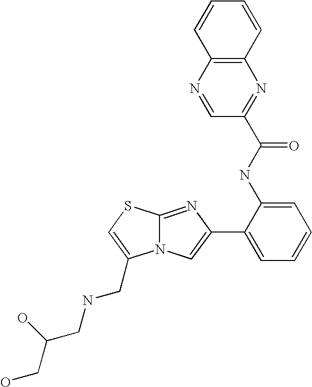 | | A′ | A |
| 677 | | 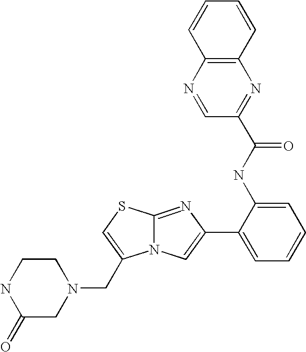 | | A′ | A |
| 678 | | 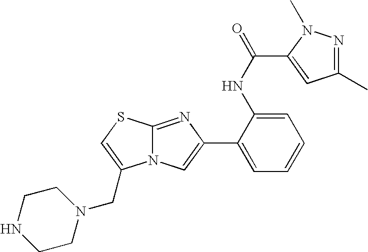 | | A | B |
| 679 | | 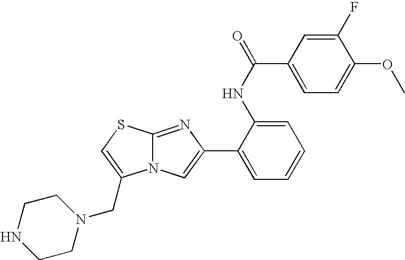 | | A | B |
| 680 | 484.2 | 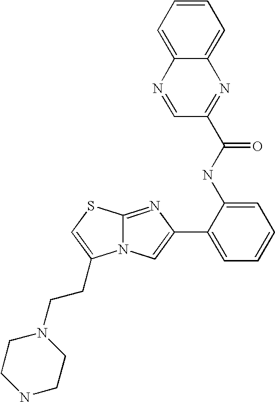 | | A′ | A |
| 681 | 522 | 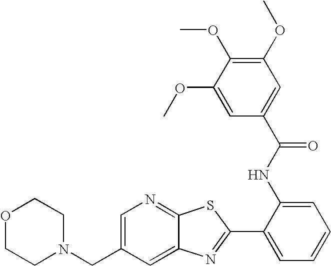 | | A | A |
| 682 | 492 | 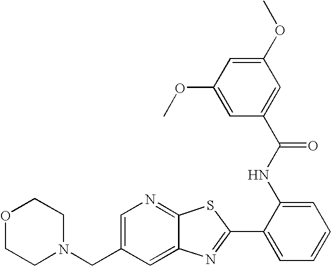 | | A′ | A |
| 683 | 475 | 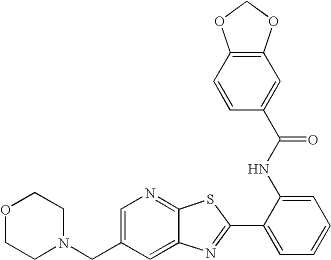 | | A | B |
| 684 | 460 | 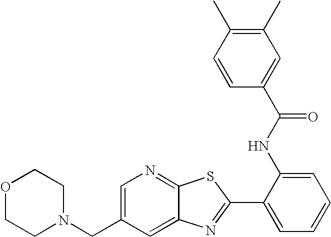 | | A | B |
| 685 | 456 | 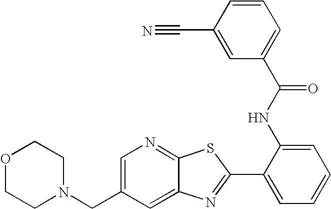 | | A′ | B |
| 686 | 475 | 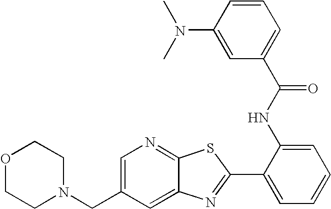 | | A′ | A |
| 687 | 467 | 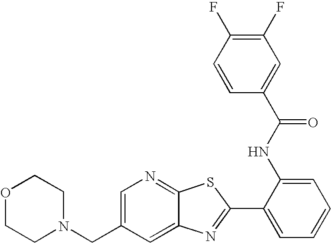 | | NA | C |
| 688 | 483 | 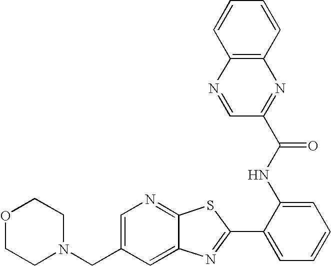 | | NA | C |
| 689 | 479 | 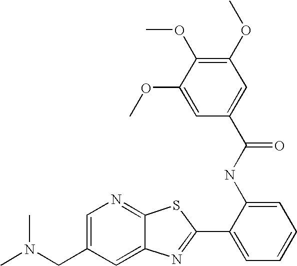 | | A | A |
| 690 | 483 | 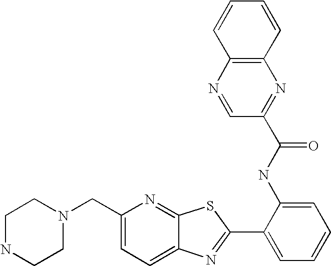 | | A′ | A |
| 692 | 469 | 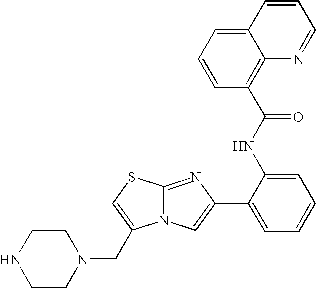 | | C | B |
| 695 | 454 | 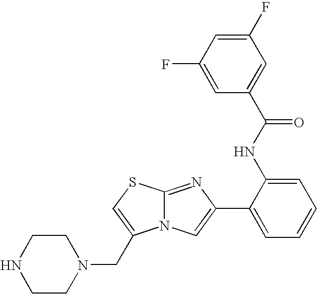 | | A′ | A |
| 697 | 596 | 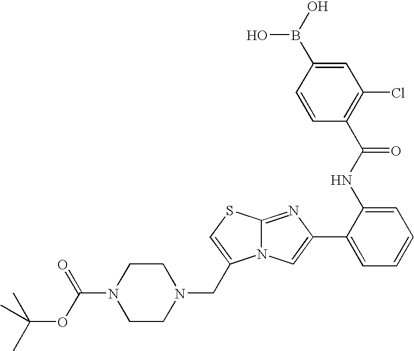 | | NA | C |
| 698 | 502 | 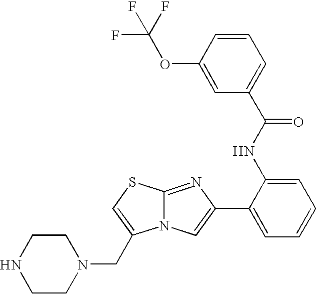 | | A′ | A |
| 699 | | 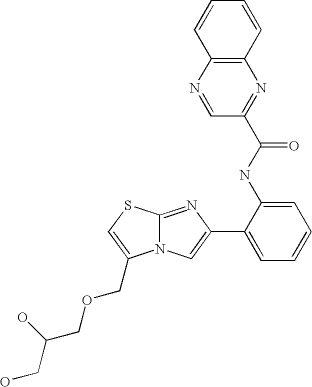 | | A | A |
| 700 | 512.2 | 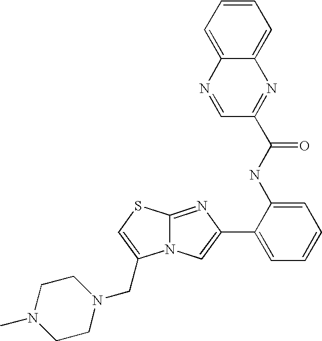 | | A′ | A |
| 701 | 477 | 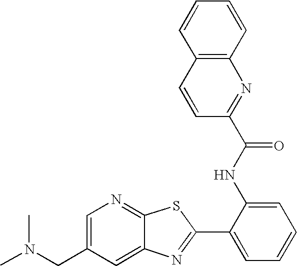 | | A′ | A |
| 702 | 534 | 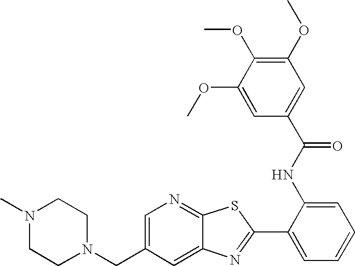 | | A | A |
| 703 | 468 | 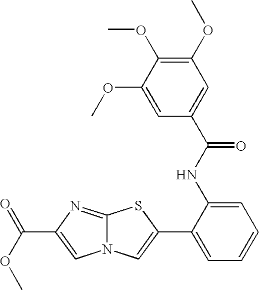 | | NA | C |
| 704 | 454 | 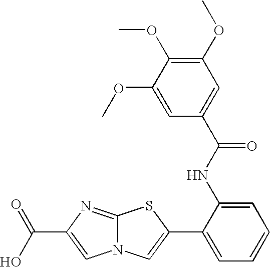 | | NA | C |
| 705 | 387 | 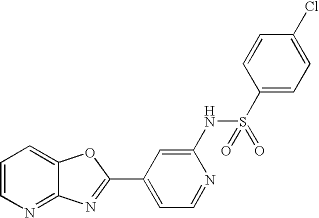 | | | |
| 706 | 445.1 | 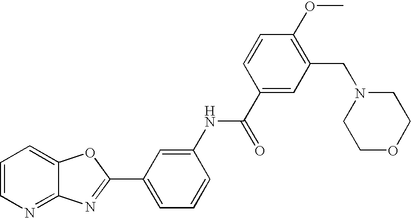 | | | |
| 707 | 386.1 | 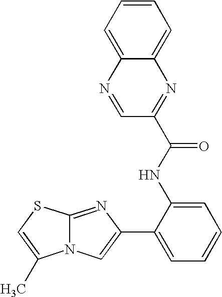 | | A′ | A |
| 708 | 494.2 | 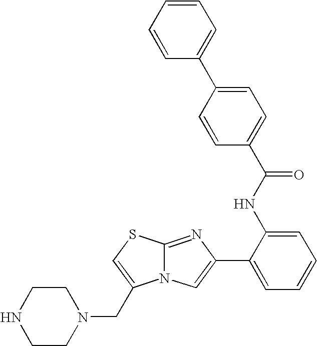 | | A′ | A |
| 709 | 494.1 | 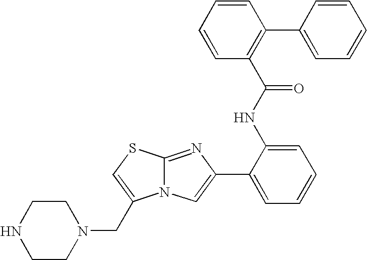 | | NA | C |
| 710 | 494.1 | 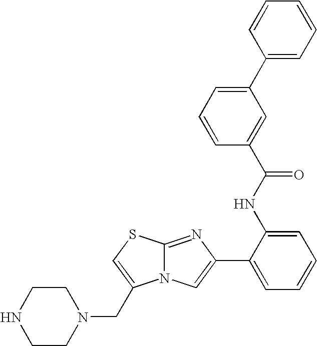 | | A′ | A |
| 711 | | 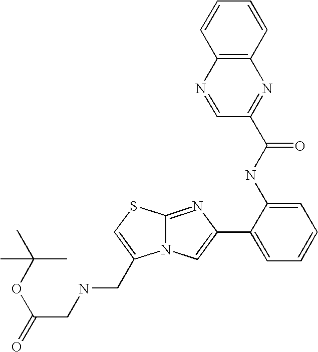 | | A | B |
| 714 | | 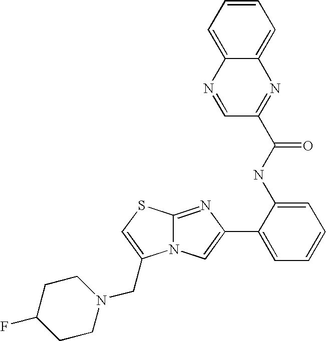 | | A | B |
| 715 | | 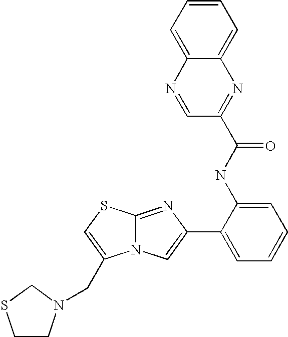 | | A′ | B |
| 716 | | 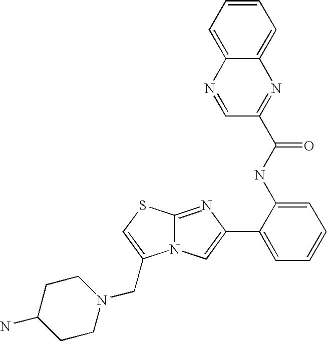 | | A′ | A |
| 717 | | 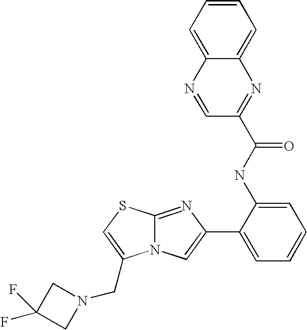 | | A | B |
| 718 | 444.1 | 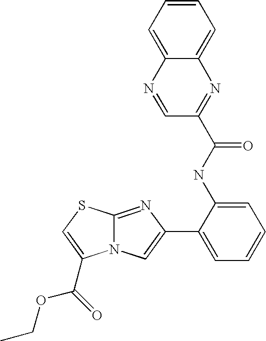 | | NA | C |
| 719 | 416 | 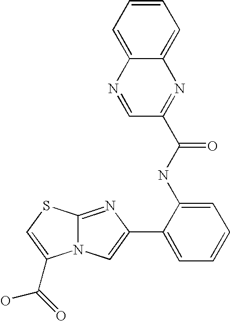 | | A | A |
| 720 | | 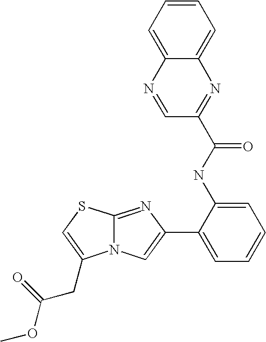 | | NA | C |
| 721 | | 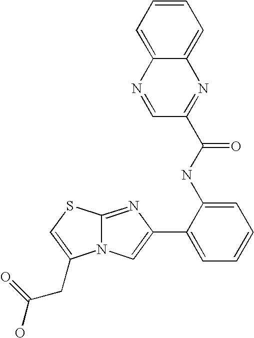 | | A | A |
| 722 | 449 | 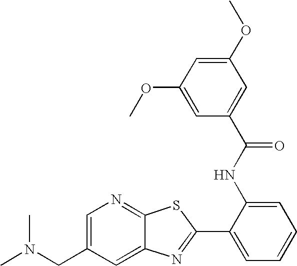 | | A′ | A |
| 723 | 490 | 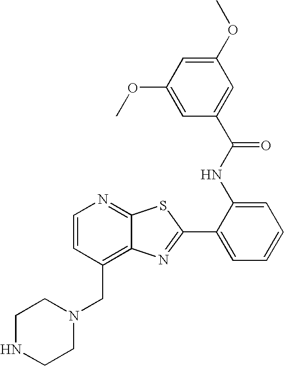 | | A | A |
| 724 | 482 | 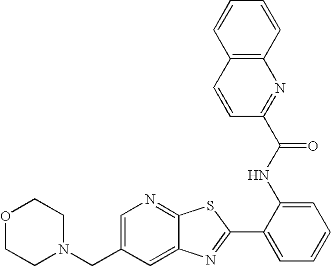 | | A′ | A |
| 725 | 505 | 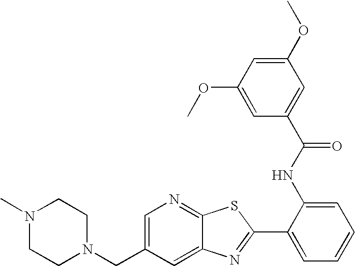 | | A′ | A |
| 726 | 491 | 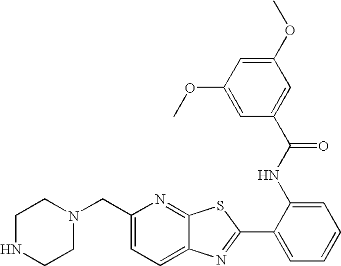 | | A′ | A |
| 727 | 458 | 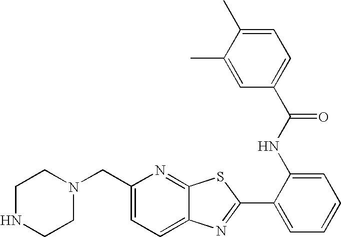 | | A′ | A |
| 728 | 466 | 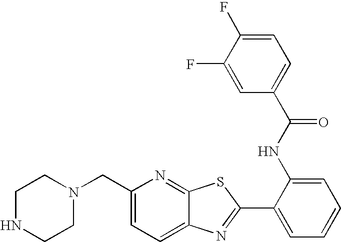 | | A | A |
| 729 | 481 | 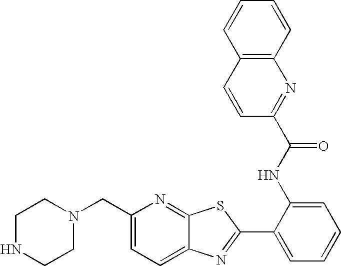 | | A′ | A |
| 730 | 488 | 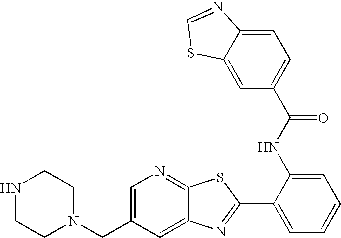 | | A | A |
| 731 | 498 | 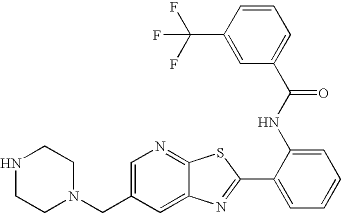 | | A | A |
| 732 | 497 | 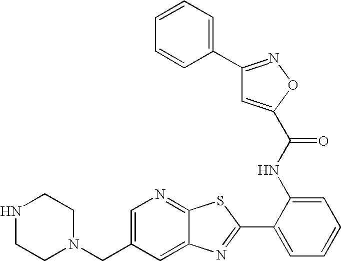 | | A′ | B |
| 733 | 484.2 | 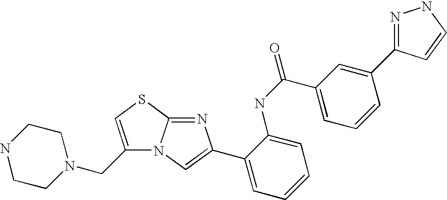 | | A′ | A |
| 735 | 514.2 | 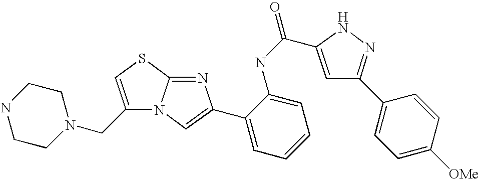 | | A′ | A |
| 736 | 514.2 | 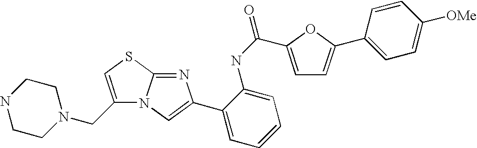 | | A′ | A |
| 737 | 500.1 | 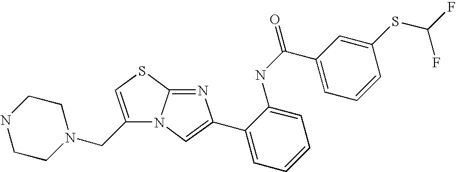 | | A′ | A |
| 738 | 448.1 | 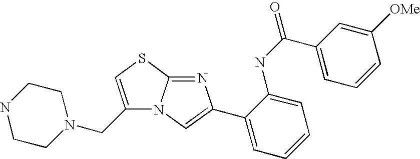 | | A | A |
| 739 | 424.1 | 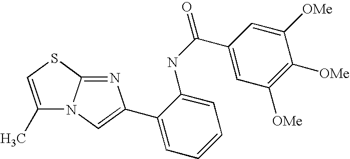 | | A′ | A |
| 740 | 377.1 | 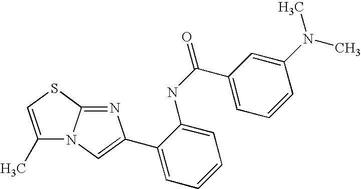 | | A′ | B |
| 741 | 498.1 | 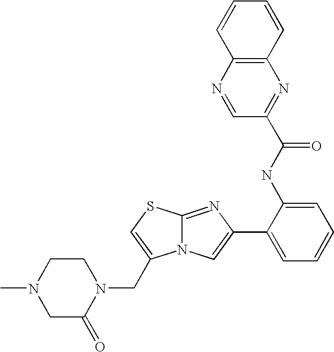 | | A′ | A |
| 742 | 487.1 | 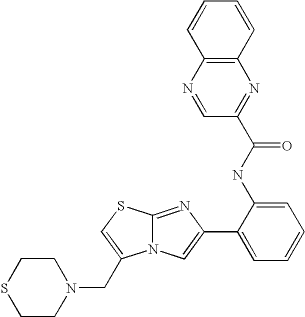 | | A′ | B |
| 743 | 466.1 | 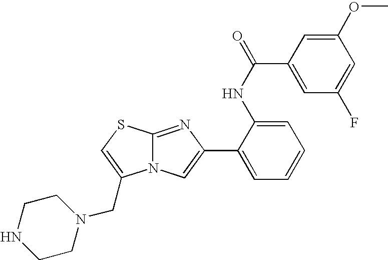 | | A | A |
| 744 | 437.1 | 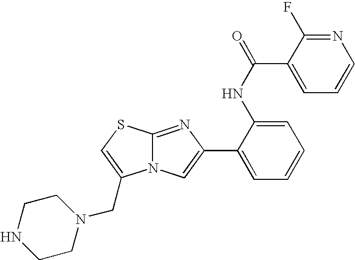 | | B | A |
| 745 | 452.1 | 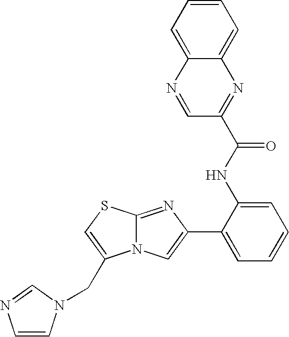 | | A′ | A |

TABLE 5

| COM- | | | | |
|---|---|---|---|---|
| POUND | | | IC₅₀ FP | IC₅₀ MS |
| NO | [M + H]+ | STRUCTURE | ASSAY | ASSAY |
| 13 | | 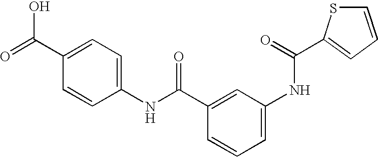 | A | |
| 14 | | 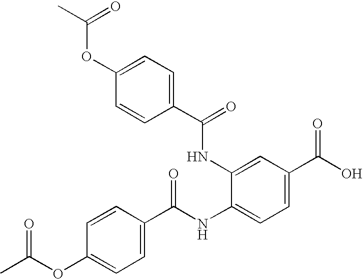 | B | |
| 15 | | 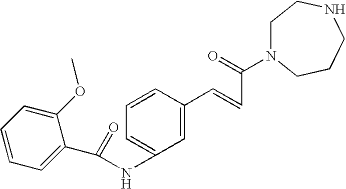 | C | |
| 23 | 359.4 | 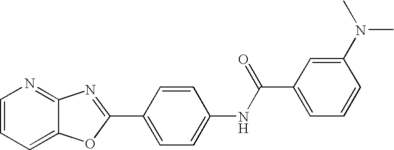 | D | |
| 25 | 341.3 | 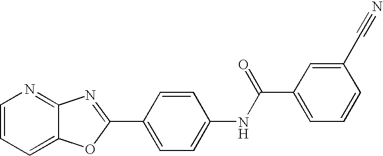 | B | |
| 26 | 341.4 | 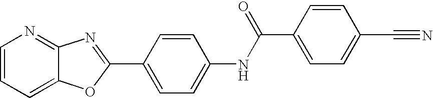 | B | |
| 28 | 401.1 | 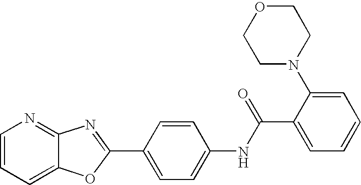 | D | |
| 30 | 380.0 | 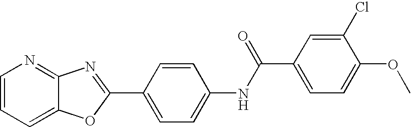 | D | |
| 44 | 341.0 | 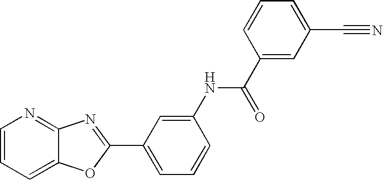 | D | |
| 47 | 385.0 | 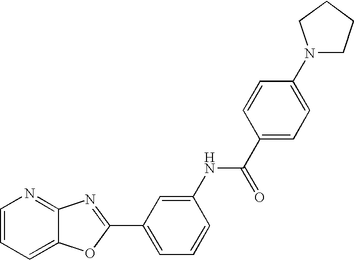 | B | |
| 291 | | 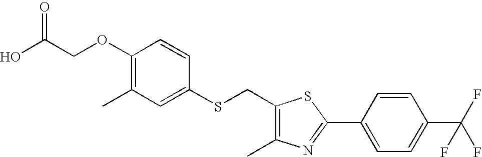 | B | |
| 652 | | 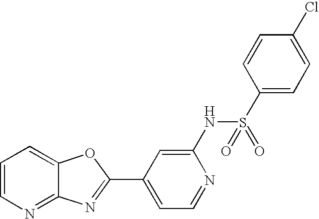 | | B |
| 653 | | 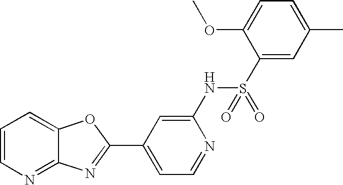 | | C |
| 654 | | 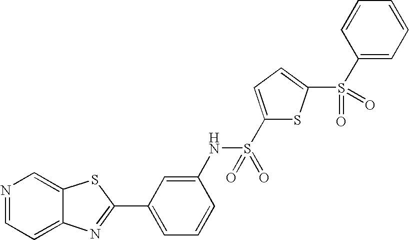 | | D |

TABLE 6

| COMPOUND NO | STRUCTURE | ED₅₀ |
|---|---|---|
| 11 | 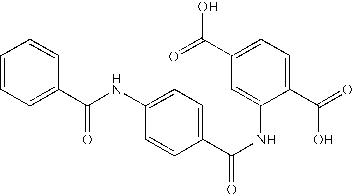 | N/A |
| 12 | 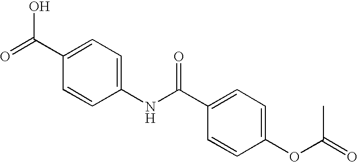 | N/A |

TABLE 7

| COMPOUND NO | STRUCTURE | IC₅₀ |
|---|---|---|
| 1 | 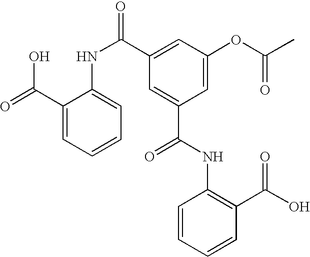 | N/A |
| 2 | 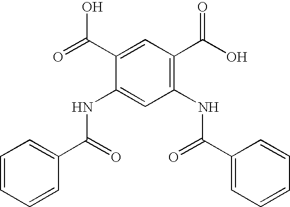 | N/A |
| 15 | 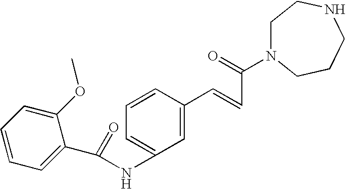 | B |
| 16 | 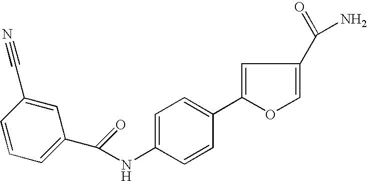 | C |
| 17 | 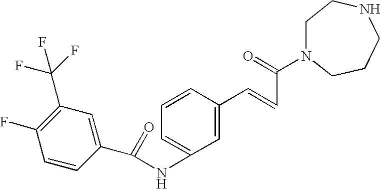 | B |
| 18 | 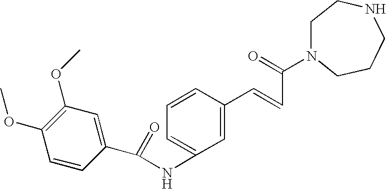 | B |

TABLE 8

| COMPOUND | | ED₅₀ ATP | IC₅₀ ATP |
|---|---|---|---|
| NO | STRUCTURE | ASSAY | ASSAY |
| 52 | 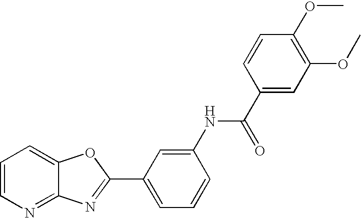 | A | |
| 42 | 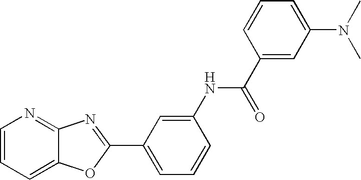 | A | |
| 49 | 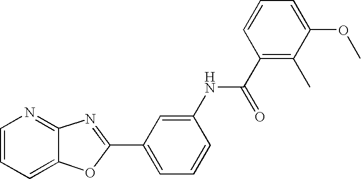 | A | |
| 115 | 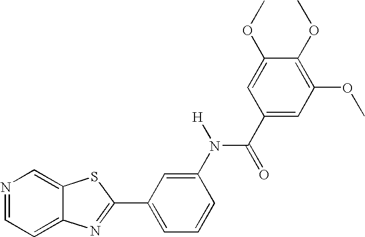 | D | |
| 79 | 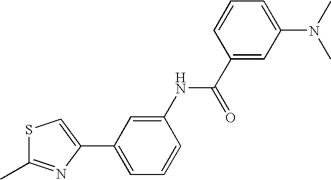 | A | |
| 117 | 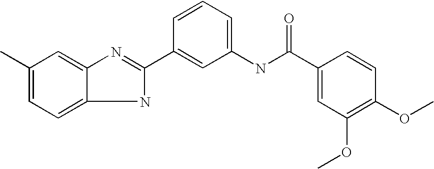 | | B |
| 120 | 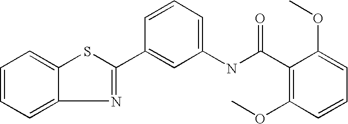 | A | |
| 121 | 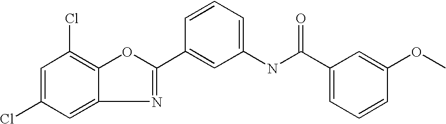 | NA | |
| 123 | 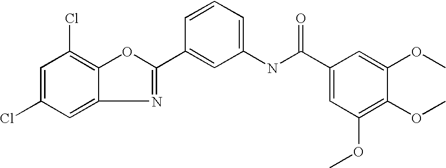 | D | |
| 85 | 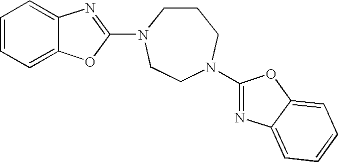 | NA | |
| 86 | 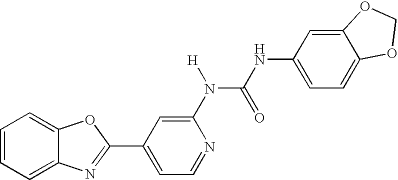 | A | |
| 87 | 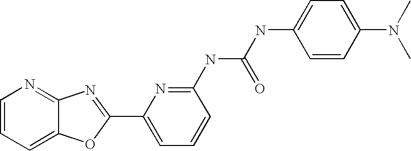 | NA | |
| 88 | 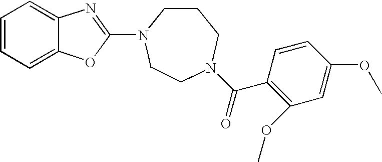 | NA | |
| 89 | 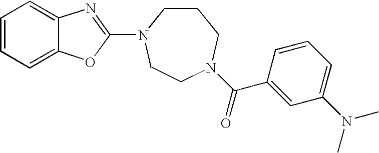 | | A |
| 90 | 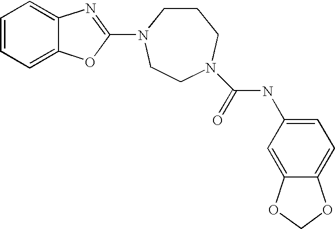 | D | |
| 91 | 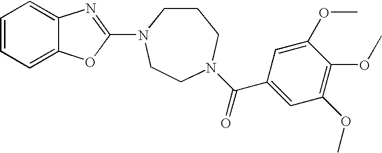 | | A |
| 92 | 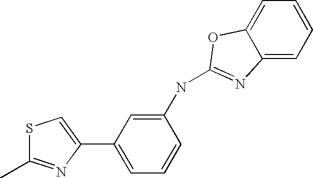 | | B |
| 93 | 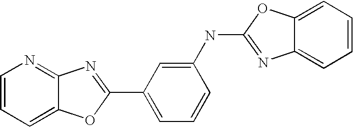 | D | |
| 94 | 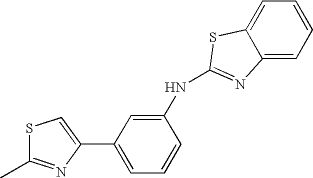 | D | |
| 95 | 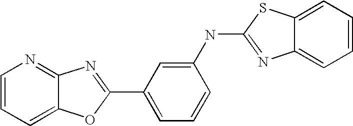 | NA | |
| 97 | 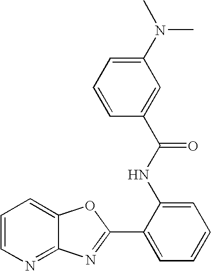 | A | |
| 98 | 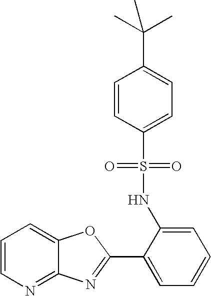 | NA | |
| 99 | 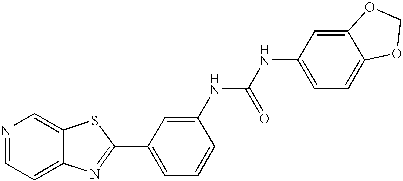 | A | |
| 100 | 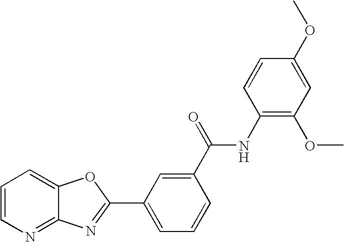 | A | |
| 101 | 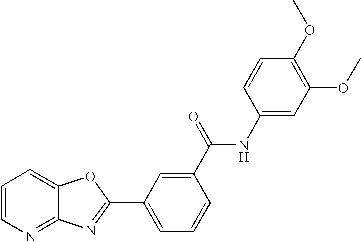 | C | |
| 102 | 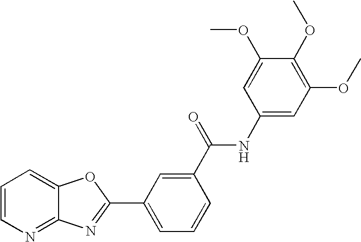 | A | |
| 103 | 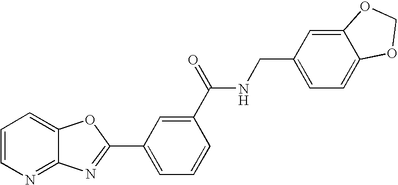 | NA | |
| 104 | 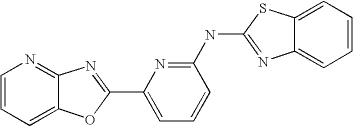 | A | |
| 105 | 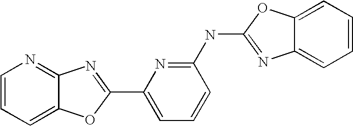 | A | |
| 133 | 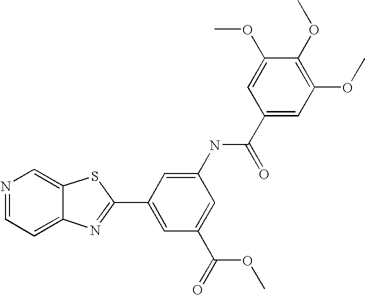 | NA | |
| 134 | 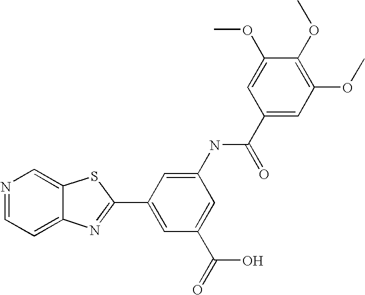 | NA | |
| 135 | 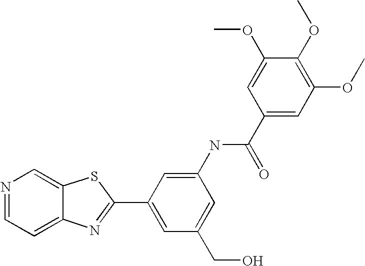 | A | |
| 106 | 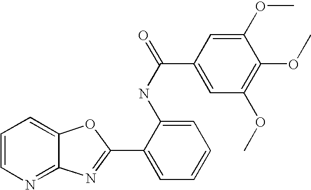 | A | |

In one embodiment, [symbol] modulating compounds of the invention are represented by Structural Formula (I):

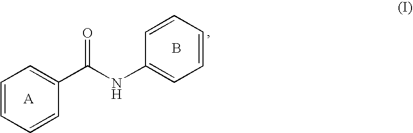

or a salt thereof, where:

Ring A is optionally substituted; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In certain embodiments, Ring B is substituted with at least a carboxy group.

In certain embodiments, Ring B is substituted with at least a substituted or unsubstituted arylcarboxamine, a substituted or unsubstituted aralkylcarboxamine or a polycyclic aryl group.

In certain embodiments, Ring B is substituted with at least a substituted or unsubstituted heteroaryl group or a substituted or unsubstituted heterocyclylcarbonylethenyl group.

In another embodiment, [symbol] -modulating compounds of the invention are represented by Structural Formula (II):

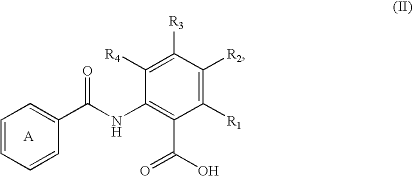

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙOR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In a further embodiment, [symbol] -modulating compounds of the invention are represented by Structural Formula (IIa):

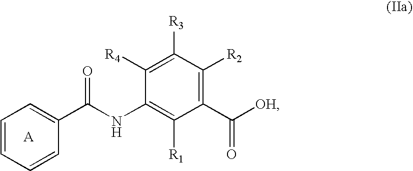

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)NR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another embodiment, [symbol] -modulating compounds of the invention are represented by Structural Formula (II):

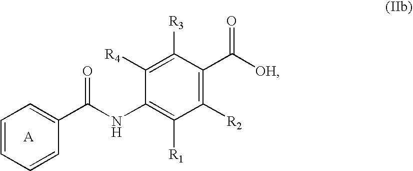

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H—, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ in Structural Formulas (II)-(IIb) are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention provides novel [symbol] modulating compounds of Formula (III):

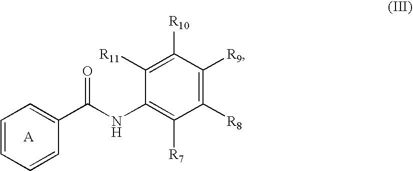

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)OR₅, —S(O)NR₅R₆, —NR₅R₆, —NR₅C(O)OR₆, —NR₅C(O)R₆ and —NO₂;

R₈ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In certain such embodiments, R₈ is selected from thiazolopyridyl, imidazothiazolyl, benzoxazinonyl, or imidazopyridyl.

Particular examples of R₈, where

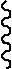

indicates attachment to the remainder of Structural Formula (III), include:

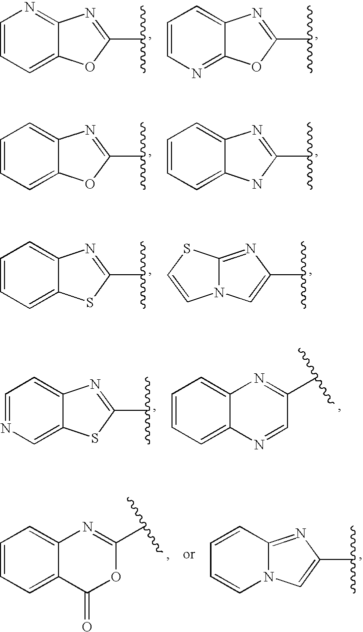

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

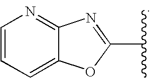

In certain embodiments, R₈ is

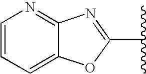

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

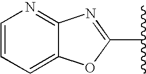

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

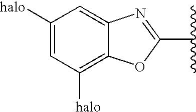

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

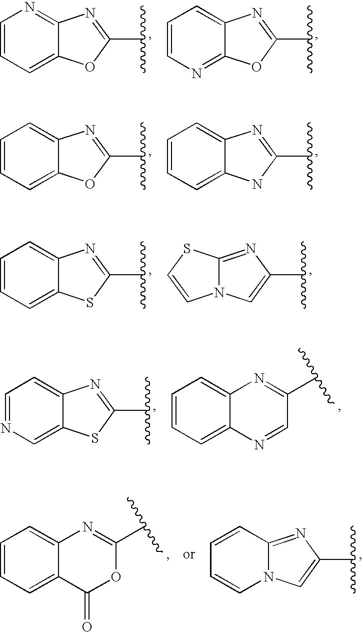

where up to 2 ring carbons not immediately adjacent to the indicated attachment point are independently substituted with C₁-C₃ straight or branched alkyl or halo; each of R₇, R₉, and R₁₀ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

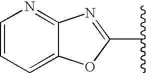

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

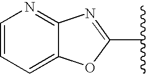

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂CH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂) methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In yet another aspect, the invention provides novel [symbol] modulating compounds of Formula (IV):

Ar-L-J-M-K-Ar′ (IV)

or a salt thereof; wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂R₂′; NR₂′C(O)NR₂′R₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or 8, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur, oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₉′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂′NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the invention, each Ar, L, and Ar′ is independently an optionally substituted 5- to 7-membered monocyclic ring system or an optionally substituted 9- to 12-membered bicyclic ring system.

According to another preferred embodiment,
Ar is

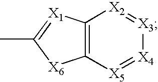

X₁, X₂, X₃, X₄, and X₅ are independently selected from CR₁′ and N; and
X₆ is selected from NR₁′, O, and S;

According to yet another preferred embodiment, X₁ and X₂ are N; X₃, X₄, and X₅ are CR₁′; and X₆ is O.

According to still yet another preferred embodiment, X₁ and X₃ are N; X₂, X₄, and X₅ are CR₁′; and X₆ is O.

According to still yet another preferred embodiment, X₁ and X₄ are N; X₂, X₃, and X₅ are CR₁′; and X₆ is O.

According to still yet another preferred embodiment, X₁ and X₅ are N; X₂, X₃, and X₄ are CR₁′; and X₆ is O.

In another embodiment, the compounds of the formula above are those wherein J is NR₁′, K is absent, and M is C(O).

In yet another embodiment, the compounds of the formula above are those wherein J is absent, K is NR₁′, and M is C(O).

In a further embodiment, compounds of formula (IV) are those where when J is absent and K is NR₁′, M is not C(O) and when J is NR₁′ and K is absent, M is not C(O).

In a preferred embodiment, the compounds above are those wherein L is an optionally substituted 5- to 7-membered carbocyclic or heterocyclic aryl group.

In yet another preferred embodiment, the compounds are those wherein L is an optionally substituted phenylene, pyridinylene, imidazolylene, oxazolylene, or thiazolylene.

In a particularly preferred embodiment, L is an optionally substituted phenylene.

In another particularly preferred embodiment, L is an optionally substituted pyridinylene.

In an even more preferred embodiment, L is phenylene.

In another even more preferred embodiment, L is pyridinylene.

In either of these embodiments, Ar and J may be attached to L at the ortho-, meta-, or para-positions. Particularly preferred are those embodiments where attachment is at the meta-position.

In certain embodiments, L is not phenylene when Ar′ is phenyl. Examples of such embodiments include embodiments where L is an optionally substituted heterocyclic aryl group and Ar′ is an optionally substituted carbocyclic or heterocyclic aryl group, or wherein L is an optionally substituted carbocyclic or heterocyclic aryl group and Ar′ is an optionally substituted heterocyclic aryl group.

In yet another aspect, the invention provides novel [symbol] -modulating compounds of Formula (I) or a salt thereof, wherein

Ring A is substituted with at least one R₁′ group;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group;

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF_{F}, OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′; and

Ring B is substituted with at least one

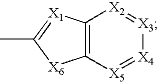

wherein

X₁, X₂, X₃, X₄, and X₅ are independently selected from CR₁′ and N; and

X₆ is selected from NR₁′, O, and S.

In a preferred embodiment, Ring B is phenyl or pyridinyl.

In a further aspect, the invention provides novel [symbol] -modulating compounds of Formula (IVa):

Het-L-Q-Ar′ (IVa)

or a salt thereof, wherein:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁═CR₁′—CR₁′R₁′—, —NR₁′—C(═N—CN)—NR₁′—, —NR₁′—C(O)—CR₁′R₁′—,

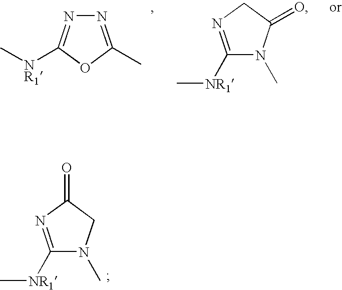

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In certain embodiments, when Het is a polycyclic heteroaryl, L is optionally substituted phenylene, and Ar′ is optionally substituted phenyl; then Q is not —NH—C(O)—.

In certain embodiments), Het and Q are attached to L in a 1-, 2- or 1-,3-configuration (e.g., when L is phenylene, Het and Q are attached in an ortho or a meta orientation). In certain embodiments where Het and Q are attached to L in a 1-,3-configuration, if Het is benzoxazolyl, L is pyridylene and Q is —NH—C(O)—NH, then Ar′ is not 3,4 dioxymethlyene phenyl; if Het is methyl thiazolyl, L is phenylene and Q is —NH—C(O)—, then Ar′ is not 3-dimethylamino phenyl; if Het is oxazolopyridyl, L is pyridylene and Q is —NH—C(O)—NH, then Ar′ is not 4-dimethylamino phenyl; if Het is oxazolopyridyl or benzoxazolyl and L is

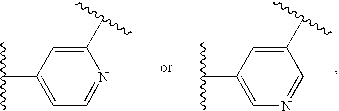

then Q is not —NH—(SO)₂—; and if Het is oxazolopyridyl, L is

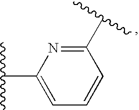

and Q is —NH—C(O)—, then Ar′ is not 3,4 dimethoxyphenyl or pyridyl.

When Het is substituted, it is typically substituted at up to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

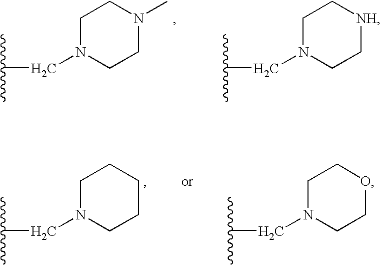

where each R₁₂ is independently selected from optionally substituted C₁-C₃ straight or branched alkyl.

In certain embodiments, Het is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In other embodiments, Het comprises one ring N heteroatom and 1 to 2 additional ring heteroatoms independently selected from N, O or S, such as thiazolyl, triazolyl, oxadiazolyl, thiazolopyridyl, imidazothiazolyl, benzoxazinonyl, or imidazopyridyl.

Particular examples of Het include:

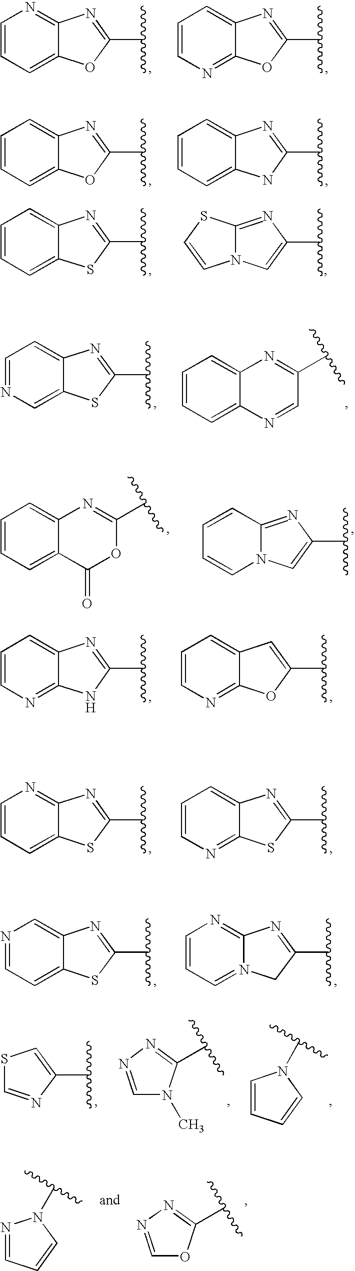

where up to 2 ring carbons not immediately adjacent to the indicated attachment point are independently substituted with optionally substituted C₁-C₃ straight or branched alkyl, phenyl, halo, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

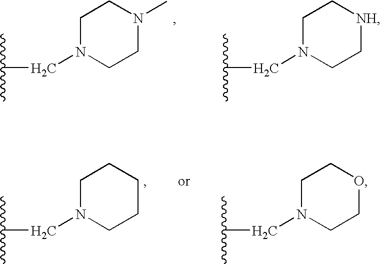

wherein each R₁₂ is independently selected from optionally substituted C₁-C₃ straight or branched alkyl.

In certain embodiments, L is selected from

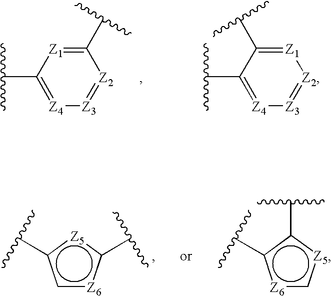

wherein:

each of Z₁, Z₂, Z₃ and Z₄ is independently selected from CH or N, wherein not more than three of said Z₁, Z₂, Z₃ or Z₄ is N;

each of Z₅ and Z₆ is independently selected from C, N, O or S, provided that at least one of Z₅ and Z₆ is N; and

L is optionally substituted at 1 to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

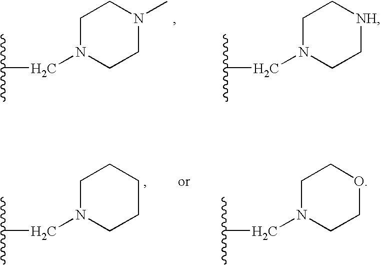

In preferred embodiments, L is selected from phenylene or pyridylene, such as unsubstituted phenylene or phenylene substituted with a single substituent selected from C(O)OCH₃, C(O)OH, CH₂OH, N(CH₃)₂, or CH₂N(CH₃)₂, or unsubstituted pyridylene.

In certain embodiments, Q is selected from —NH—C(O)—, —NH—S(O)—, —NH—C(O)—NH—, —C(O)—NH—, —CH₂—, —N(CH₃)—C(O)—NH—, —NH—C(O)—N(CH₃)—, or —NH—S(O)₂—NH—, particularly —NH—C(O)—, —C(O)—NH—, —NH—, —NH—C(O)—NH, or —NH—S(O)₂—.

In certain embodiments, Ar′ is selected from optionally substituted phenyl, benzothiazolyl, or benzoxazolyl. When Ar′ is phenyl, typical optional substituents are 1 to 3 substituents independently selected from halo, (optionally substituted C₁-C₃ straight or branched alkyl), O-(optionally substituted C₁-C₃ straight or branched alkyl), S-(optionally substituted C₁-C₃ straight or branched alkyl), N(CH₃)₂ or optionally substituted heterocyclyl, or wherein two substituents on adjacent ring atoms are taken together to form a dioxymethylene.

In certain embodiments, Het is selected from

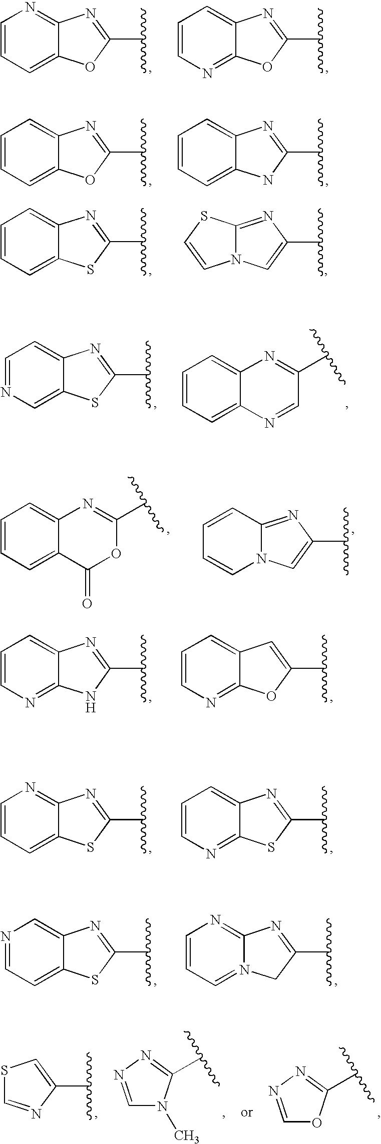

and wherein up to 2 ring carbons not immediately adjacent to the indicated attachment point are independently substituted with optionally substituted C₁-C₃ straight or branched alkyl, phenyl or halo;

L is selected from unsubstituted phenylene, phenylene substituted with a single substituent selected from C(O)OCH₃, C(O)OH, CH₂OH, N(CH₃)₂, or CH₂N(CH₃)₂, or unsubstituted pyridylene;

Q is selected from —NH—C(O)—, —C(O)—NH—, —NH—, —NH—C(O)—NH, or —NH—S(O)₂—; and

Ar′ is selected from optionally substituted phenyl, benzothiazolyl, or benzoxazolyl, wherein said phenyl is optionally substituted with 1 to 3 substituents independently selected from chloro, methyl, O-methyl, S-methyl, N(CH₃)₂, morpholino, or 3,4 dioxymethylene.

In certain embodiments, Q is selected from —NH—C(O)—, —C(O)—NH—, —NH— or NH—C(O)—NH.

In certain embodiments, the substituents on Ar′ are selected from chloro, methyl, O-methyl, S-methyl or N(CH₃)₂. In certain embodiments, the only substituent on Ar is an O-methyl group, particularly an O-methyl group ortho or meta to Q. In certain embodiments, when there are two or more O-methyl groups or Ar′, at least one is ortho or meta to Q.

In certain embodiments, L is pyridyl and Het and Q are at the 1,3- or 2,4-position with respect to the pyridyl nitrogen atom. In certain such embodiments, Q is —NH—S(O)—.

In certain embodiments where L is further substituted, the substituent is typically meta to both Het and Q.

In certain embodiments, Q is —NH— and Het is thiazolyl or oxazolopyridyl.

In certain embodiments, Q is —NH— and Ar is benzothiazolyl or benzoxazolyl.

In certain embodiments, such as when the [symbol] -modulator is,

[symbol] , L is

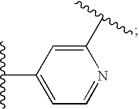

and Q is —NH—(SO)₂—. In certain such embodiments, Het is oxazolopyridyl. When L, Q and optionally Het have these values, Ar′ is advantageously naphthyl or phenyl, where Ar′ is optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments, such as when the [symbol] modulator is,

[symbol] , L is

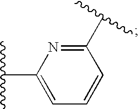

and Q is —NH—C(O)—. In certain such embodiments, Het is oxazolopyridyl. When L, Q and optionally Het have these values, Ar′ is advantageously pyridyl or phenyl optionally substituted with 1 to 3 substituents independently selected from CN, halo, (C1-C3 straight or branched alkyl), O—(C1-C3 straight or branched alkyl), N(C1-C3 straight or branched alkyl)₂, or a 5 to 6-membered heterocycle.

In certain embodiments,
Het comprises one N heteroatom and 1 to 2 additional heteroatoms independently selected from N, O or S;

L is

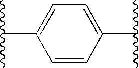

and is optionally substituted;

Q is —NH—C(O)—; and

Ar′ is phenyl substituted with 1 to 3 substituents independently selected from CN, halo, C₁-C₃ straight or branched alkyl, O—(C₁-C₃ straight or branched alkyl), N(C₁-C₃ straight or branched alkyl)₂, or a 5 to 6-membered heterocycle, wherein when R₈ is unsubstituted

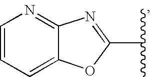

then ring A is:
a) not simultaneously substituted at the 2- and 6-positions with O—(C₁-C₃ straight or branched alkyl);
b) not simultaneously substituted at the 2-position with C₁-C₃ straight or branched alkyl or O—(C₁-C₃ straight or branched alkyl) and at the 3-position with O—(C₁-C₃ straight or branched alkyl);
c) not substituted at the 4-position with O—(C₁-C₃ straight or branched alkyl) unless simultaneously substituted at the 3-position with halo or O—(C₁-C₃ straight or branched alkyl) and unsubstituted at all other positions;
   not substituted at the 4-position with N(C₁-C₃ straight or branched alkyl)₂, or said 5 to 6-membered heterocycle. In certain such embodiments, L is unsubstituted and/or Het is oxazolopyridyl.

In yet another aspect, the invention provides novel: [symbol] modulating compounds of Formula (V):

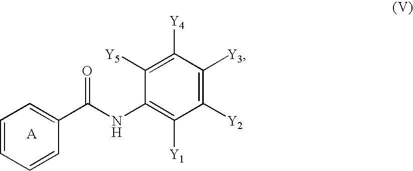

or a salt thereof, wherein:

Ring A is optionally substituted with at least one R₁′ group;

Y₁, Y₂, Y₃, Y₄, and Y₅ are independently R₁′;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′, C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₉′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the above compound,

either Y₂ or Y₃ is

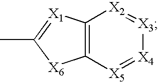

X₁, X₂, X₃, X₄, and X₅ are independently selected from CR₁′ and N; and

X₆ is selected from NR₁′, O, and S.

According to an even more preferred embodiment, X₁ and X₂ are N; X₃, X₄, and X₅ are CR₁′; and X₆ is O.

According to another even more preferred embodiment, X₁ and X₃ are N; X₂, X₄, and X₅ are CR₁′; and X₆ is O.

According to another even more preferred embodiment, X₁ and X₄ are N; X₂, X₃, and X₅ are CR₁′; and X₆ is O.

According to another even more preferred embodiment, X₁ and X₅ are N; X₂, X₃, and X₄ are CR₁′; and X₆ is O.

In another aspect, the invention provides [symbol] -modulating compounds of Structural Formula (VII):

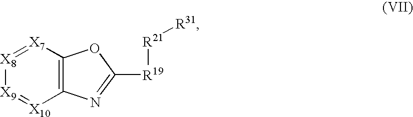

or a salt thereof, wherein:

each of X₇, X₈; X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

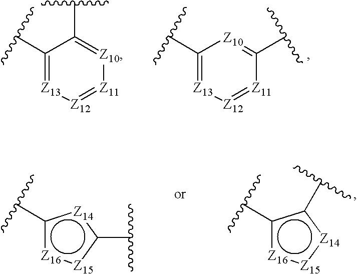

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

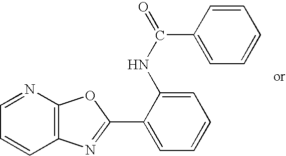

that when R¹⁹ is

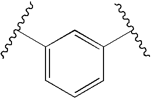

and R²¹ is —NHC(O)—, R³¹ is not an optionally substituted phenyl.

In certain embodiments, compounds of Structural Formula (VII) have the following values:

each of X, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²¹ or CR₁′; and

zero to one R²⁰ is a solubilizing group;

R¹⁹ is selected from:

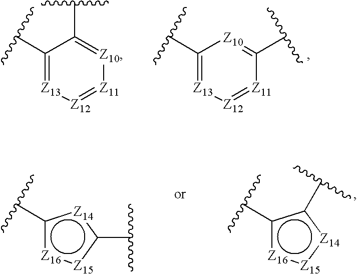

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

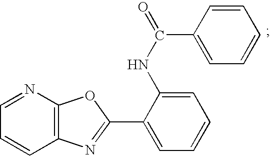

and

when X₈ and X₉ are each independently selected from CR²⁰ or CR₁′, R¹⁹ is

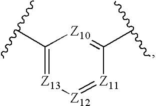

and each of Z₁₀, Z₁₁, Z₁₂ and Z₁₃ is independently selected from CR²⁰, or CR₁′, then:

a) at least one of X₈ and X₉ is not CH; or

b) at least one of Z₁₀, Z₁₁, Z₁₂ and Z₁₃ is CR²⁰, wherein R²⁰ is a solubilizing group.

In certain embodiments, when Z₁₂ is CR²⁰ and R²⁰ is a solubilizing group, the solubilizing group is not —C(O)OCH₂CH₃, —COOH,

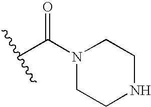

or

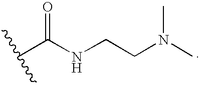

In certain embodiments, when X₈ and X₉ are each independently selected from CR²⁰ or CR₁′, R¹⁹ is

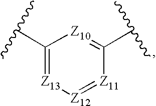

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁ and Z₁₃ is CR²⁰, wherein R²⁰ is a solubilizing group.

In certain embodiments, when R¹⁹ is

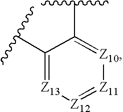

and each of Z₁₀, Z₁₁, Z₁₂ and Z₁₃ is CR²⁰, or CR₁′; X₈ and X₉ are CR²⁰ or CR₁′; R²¹ is —NHC(O)—; and R³¹ is optionally substituted phenyl, then R³¹ is a substituted phenyl, at least one R₁′ in a CR₁′ moiety is optionally substituted C₁-C₃ straight or branched alkyl, or at least one R²⁰ in a CR²⁰ is a solubilizing group, or a combination thereof.

In certain embodiments, R¹⁹ is selected from phenyl, pyridyl, thienyl or furyl.

In certain embodiments, R¹⁹ is

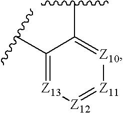

wherein each of Z₁₀, Z₁₁, Z₁₂ and Z₁₃ is independently selected from CR²⁰ or CR₁′; and

R²¹ is —NH—C(O)—; and

R³¹ is a substituted phenyl.

In certain such embodiments, when X₉ is N, R³¹ is not 2,4 dimethoxyphenyl and/or when X₁₀ is N, R³¹ is not halo substituted phenyl; 3,4-dioxoethylenephenyl; or 3,5-dimethoxyphenyl.

In preferred embodiments, R³¹ is optionally substituted with 1 to 3 substituents independently selected from —OCH₃, —CH₃, —N(CH₃)₂, pyrazinoxy or a solubilizing group. Suitable examples of R³¹ include 3-methoxy-4-((4-methylpiperazin-1-yl)methyl)phenyl, 3-methoxy-4-morpholinomethylphenyl, 3-methoxy-4-diaminomethylphenyl, 3-methoxy-4-((pyrrolidin-1-yl)methyl)phenyl, 3,4-dimethoxyphenyl, 3,5-dimethoxyphenyl, 2,3,4-trimethoxyphenyl, 3,4,5-trimethoxyphenyl, 2-dimethylaminophenyl, 3-dimethylaminophenyl, 4-dimethylaminophenyl, or 3,5-dimethylphenyl.

In certain embodiments, R¹⁹ is selected from

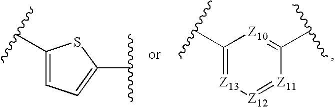

wherein one of Z₁₀, Z₁₁, Z₁₂, and Z₁₃ is N and the others are independently selected from CR²⁰ or CR₁′;

R²¹ is selected from —NH—, —NH—C(O)—, —NH—C(O)—NH, —NH—C(S)—NH— or —NH—S(O)₂—; and

R³¹ is selected from an optionally substituted phenyl, an optionally substituted naphthyl, or an optionally substituted heteroaryl.

In certain such embodiments,
a) when R²¹ is —NH—S(O)₂—, either.
   i) Z₁₀ is N; or
   ii) Z₁₁ is N and R³¹ is halophenyl or 2-methoxy-5-methylphenyl;
b) when R¹⁹ is

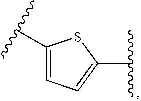

R³¹ is not 4-dimethylaminophenyl, 2,3,4-trimethoxyphenyl, or 3,5 dimethoxyphenyl; and/or
c) when R²¹ is —NH—C(O)—NH— and Z₁₀ is N, R³¹ is not 4-dimethylaminophenyl.

In certain such embodiments, R³¹ is selected from optionally substituted phenyl, benzothiazolyl, or benzoxazolyl.

In yet another embodiment, the invention provides: [symbol] modulating compounds of Structural Formula (VIII):

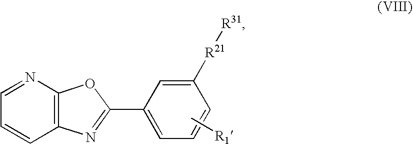

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

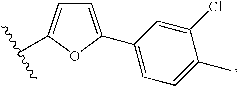

1-methoxynaphthyl, 2-methoxynaphthyl, or unsubstituted 2-thienyl;

when R₁′ is methyl, and R²¹ is —NH—C(O)—CH═CH—, R³¹ is not

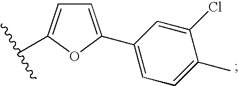

when R₁′ is methyl, and R²¹ is —NH—C(O)—CH—O—, R³¹ is not unsubstituted naphthyl, 2-methoxy, 4-nitrophenyl, 4-chloro-2-methylphenyl, or 4-t-butylphenyl; and

when R²¹ is —NH—C(O)—, R³¹ is not optionally substituted phenyl.

In certain embodiments, R²¹ is —NH—C(O)—; and R³¹ is phenyl optionally substituted with 1 to 3 substituents independently selected from —OCH₃, —CH₃, —N(CH₃)₂, or a solubilizing group.

In certain such embodiments, R²¹ is —NH—C(O)— and R³¹ is selected from unsubstituted phenyl, 2-methoxyphenyl, 3-methoxyphenyl, 2,3,4-trimethoxyphenyl, 3,4,5-trimethoxyphenyl; 2,4-dimethoxyphenyl, 3,5-dimethoxyphenyl, 2-methyl-3-methoxyphenyl, 2-morpholinophenyl, 2-methoxy-4-methylphenyl, 2-dimethylaminophenyl, 4-dimethylaminophenyl, or

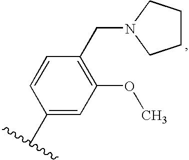

particularly phenyl; 2-methoxyphenyl; 3-methoxyphenyl; 2,3,4-trimethoxyphenyl; 3,4,5-trimethoxyphenyl; 2,4-dimethoxyphenyl; 3,5-dimethoxyphenyl; 2-methyl-3-methoxyphenyl; 2-morpholinophenyl; 2-methoxy-4-methylphenyl; 2-dimethylaminophenyl; or 4-dimethylaminophenyl.

In a further embodiment, the invention provides [symbol] -modulating compounds of Structural Formula (IX):

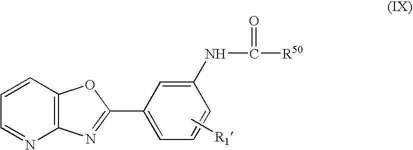

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] -modulating compounds of Structural Formula (X):

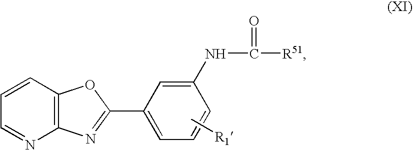

or a salt thereof, wherein:

R₁′ is selected from H or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In certain embodiments, R⁵¹ is selected from pyrazolyl, thiazolyl, oxazolyl, pyrimidinyl, furyl, thienyl, pyridyl, isoxazolyl, indolyl, benzopyrazolyl, benzothiazolyl, benzoxazolyl, quinoxalinyl, benzofuranyl, benzothienyl, quinolinyl, benzoisoxazolyl, benzotriazinyl, triazinyl, naphthyl, or

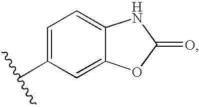

and wherein R⁵¹ is optionally substituted. In certain such embodiments, R⁵¹ is selected from pyrazolyl thiazolyl, oxazolyl, pyrimidinyl, indolyl, pyrazinyl, triazinyl, or

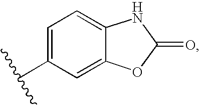

and R⁵¹ is optionally substituted.

In another aspect, the invention provides [symbol] -modulating compounds of Structural Formula (XI):

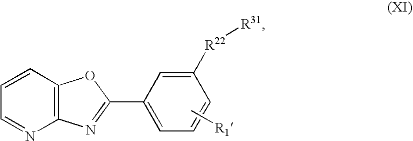

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′, —NR₁′—C(O)—CR₁′R₁′—NR₁′—, —NR₁′—C(═NR₁′)—NR₁′—, C(O)—NR₁′—, —C(O)—NR₁′—S(O)₂—, —NR₁′—, —CR₁′R₁′—, —NR₁′—C(O)—CR₁═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl; 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is ═NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In certain embodiments, R²² is selected from —C(O)—NH—, —NH—, or —C(O)—NH—CH₃.

In certain embodiments, such as when R²² is selected from —C(O)—NH—, —NH—, or —C(O)—NH—CH₃, R³¹ is selected from optionally substituted phenyl, benzothiazolyl, quinoxalinyl, or benzoxazolyl.

In yet another aspect, the invention provides [symbol] -modulating compounds of Structural Formula (XII):

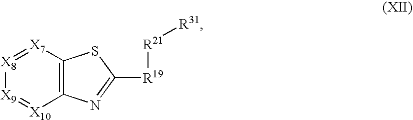

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

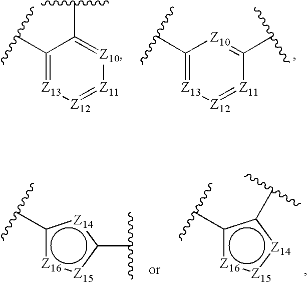

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O— —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that when R¹⁹ is

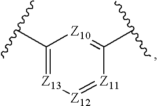

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

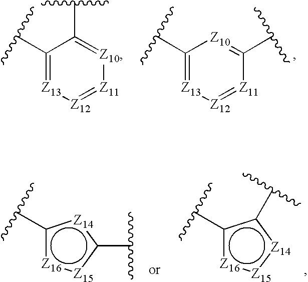

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₃′, wherein:
zero to two of Z₁₀, Z₁₁, Z₁₂ or Z₁₃ are N;
at least one of Z₃₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

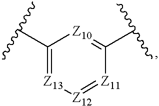

and each of Z₁₀, Z₁₁, Z₁₂ and Z₁₃ is independently selected from C²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In certain embodiments, R²¹ is —NH—C(O)— and R¹⁹ is selected from:

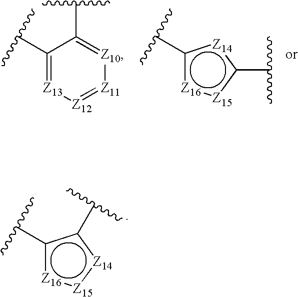

In certain embodiments, R¹⁹ is selected from optionally substituted phenyl, optionally substituted pyridyl, optionally substituted thienyl or optionally substituted furyl.

In certain embodiments, R¹⁹ is

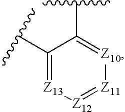

wherein each of Z₁₀, Z₁₁, Z₁₂ and Z₁₃ is independently selected from CR²⁰ or CR₁′; and

R²¹ is selected from —NH—C(O)—, —NH—C(O)—CH(CH₃)—O—, —NH—C(O)—CH₂—O—, or —NH—S(O)₂—CH₂—CH₂—; and

R³¹ is selected from an optionally substituted aryl, or an optionally substituted heteroaryl.

In certain such embodiments, R³¹ is optionally substituted with 1 to 3 substituents independently selected from —OCH₃, —CH₃, —N(CH₃)₂, phenyl, phenoxy, 3,4-dioxymethylene, fluoro, or another solubilizing group. Suitable examples of R³¹ include unsubstituted quinolinyl, 2,4-dimethoxyphenyl, 3,4-dimethoxyphenyl, 3,5-dimethoxyphenyl, 3,4,5-trimethoxyphenyl, 2,3,4-trimethoxyphenyl, 2-dimethylaminophenyl, 3-dimethylaminophenyl, 4-dimethylaminophenyl, 3,5-dimethylphenyl, 3,5-difluorophenyl, 3-trifluoromethoxyphenyl, unsubstituted quinoxalinyl, unsubstituted benzopyrimidinyl,

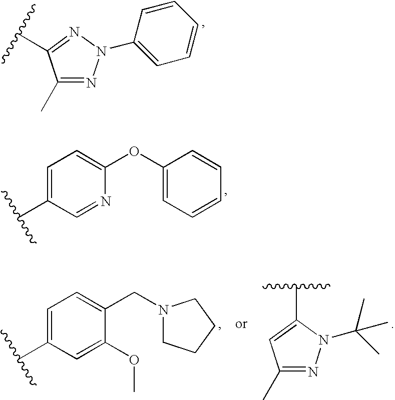

In certain such embodiments, R³¹ is not phenyl-substituted furyl.

In certain embodiments, R¹⁹ is selected from

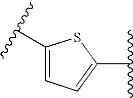

or

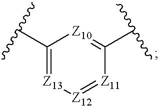

each of Z₁₀, Z₁₁, Z₁₂ and Z₁₃ is independently selected from CR²⁰, or CR₁′;

R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

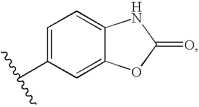

and R³¹ is optionally substituted (e.g., optionally substituted with up to three substituents independently selected from —OCH₃, —CH₃, —N(CH₃)₂, —O-phenyl, or another solubilizing group). Suitable examples of R³¹ include unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

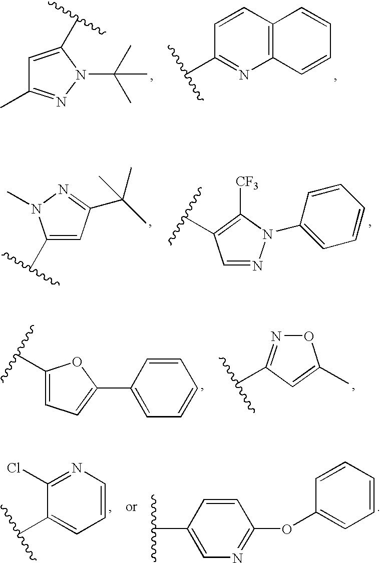

In certain embodiments, one or more of the following conditions applies:

when X₈ is N, R²¹ is —NH—C(S)—NH—, and R¹⁹ is phenyl, R³¹ is not 2-methoxy-5-nitrophenyl, 2-S-methylphenyl or 2-acetylphenyl;

when X₈ is N, R²¹ is —NH—S(O)₂—, and R¹⁹ is phenyl, R³¹ is not thiadiazole-substituted thienyl or 4-methylsulfonylphenyl;

when X₈ is N, R²¹ is —NH—CO—, and R¹⁹ is phenyl, R³¹ is not 2,4-difluorophenyl, pyridyl-substituted thienyl, 3,4-dichlorophenyl, 4-t-butylphenyl or 3-benzyloxyphenyl;

when X₉ is N, R²¹ is —NH—C(O)— and R¹⁹ is

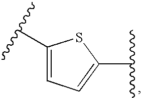

R³¹ is not 2,3,4-trimethoxyphenyl or 3,5-dimethoxyphenyl; and

when X₉ is N, R²¹ is —NH—C(O)— and R¹⁹ is phenyl, R³¹ is not 3,5-dimethoxyphenyl.

In a further embodiment, the invention provides compounds of Structural Formula (XIII):

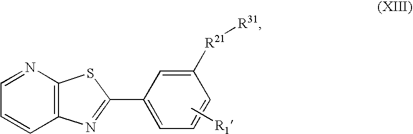

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹³ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In certain embodiments, R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from a monocyclic or bicyclic aryl or a monocyclic or bicyclic heteroaryl, and comprises a solubilizing group substituent.

In certain embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

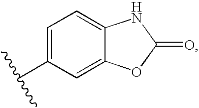

and R³¹ is optionally substituted.

In certain embodiments, R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, particularly when R²¹ is —NH—C(O)—, R³¹ is selected from R³¹ is selected from unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

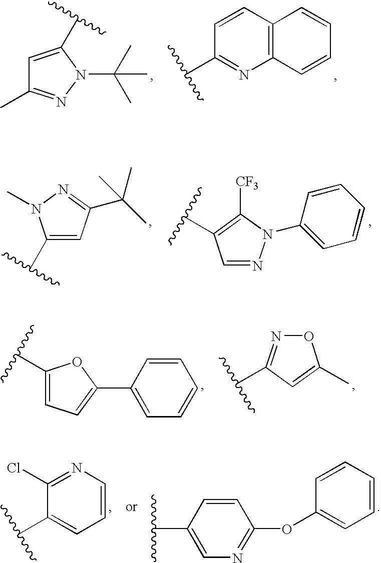

In one aspect, the invention provides [symbol] -modulating compounds of Structural Formula (XIV):

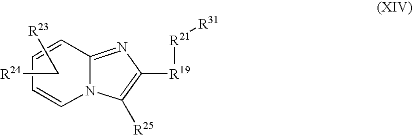

or a salt thereof, wherein:

each of R²³ and R²⁴ is independently selected from i, —CH₃ or a solubilizing group;

R²⁵ is selected from H or a solubilizing group; and

R¹⁹ is selected from:

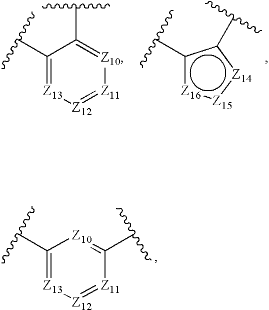

or

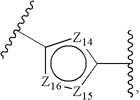

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′ O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

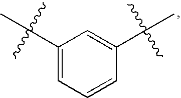

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In certain embodiments, each of R²³ and R²⁴ is independently selected from H, —CH₃ or a solubilizing group;

R²⁵ is selected from H, or a solubilizing group; and

R¹⁹ is selected from:

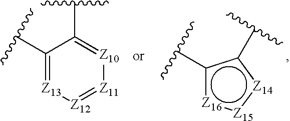

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₁ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, R³¹ is not 2,4-dimethoxyphenyl.

Typically, R²⁵ is selected from H, —CH₂—N(CH₃)₂, or

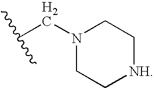

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In certain embodiments, each of R²³ and R²⁴ is independently selected from H, —CH₃ or a solubilizing group;

R²⁵ is selected from H, or a solubilizing group; and

R¹⁹ is selected from:

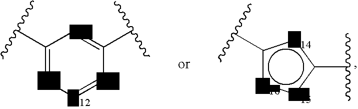

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ is N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′, —NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′, —C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is phenyl, at least one of R²³, R²⁴, or R²⁵ is a solubilizing group and wherein said compound is not 2-chloro-N-[3-[3-(cyclohexylamino)imidazo[1,2-a]pyridin-2-yl]phenyl]-4-nitrobenzamide.

Typically, R²⁵ is selected from H, —CH₂—N(CH₃)₂, or

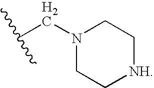

Typically, R²³ and R²⁴ are H.

Typically, R¹⁹ is selected from phenyl, pyridyl, thienyl or furyl, particularly optionally substituted phenyl. Preferably, a phenyl is optionally substituted with:

b) up to three —O—CH₃ groups; or

b) one —N(CH₃)₂ group.

In another aspect, the invention provides [symbol] -modulating compounds of Structural Formula (XV):

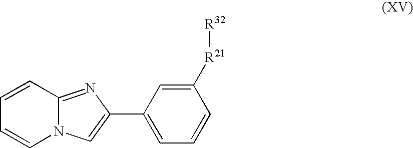

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′R₁′—, —C(O)—NR₁′—, —C(O)—NR₁′—S(O)₂—, —NR₁′—, —CR₁′R₁′—, —NR₁′—C(O)—CR₁′═CR₁′—; —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₂′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

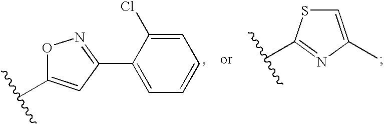

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

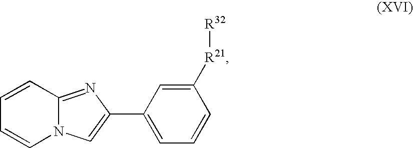

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, —NR₁′—C(═N—CN)—NR₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In certain embodiments, R²¹ is selected from —NR¹²—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═—R₁′—CR₁′R₁′—, —NR₁′—C(═N—CN)—NR₁′—, or —NR₁′—C(O)—CR₁′R₁′—; and

each R₁′ is independently selected from H or optionally substituted C₁-C₃ straight or branched alkyl;

R²² is an optionally substituted C₁-C₃ straight or branched alkyl; and

R³³ is phenyl comprising a solubilizing group substituent, wherein: when R²¹ is —NH—S(O)₂ said phenyl comprises an additional substituent.

In certain embodiments, R²¹ is selected from —NR²²—C(O)—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, or —NR₁′—C(═N—CN)—NR₁′—,

each R₁′ is independently selected from H or optionally substituted C₁-C₃ straight or branched alkyl; and

R²² is an optionally substituted C₁-C₃ straight or branched alkyl.

In certain embodiments, R³³ is optionally substituted on up to three carbon atoms with a substituent independently selected from —O—CH₃, —CH₃, —N(CH₃)₂, —S(CH₃), or CN; or substituted on adjacent carbon atoms with

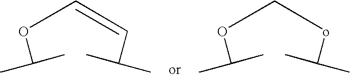

bridging said adjacent carbon atoms.

In a further embodiment, the invention provides [symbol] -modulating compounds of Structural Formula (XVII):

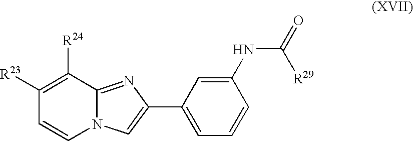

or a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position,

wherein R²⁹ is optionally additionally substituted with a solubilizing group.

In certain embodiments, R²⁹ is phenyl substituted with:

a) three —O—CH₃ groups located at the 2, 3 and 4 positions; or

b) one —N(CH₃)₂ group.

In one aspect, the invention provides [symbol] -modulating compounds of Structural Formula (XVIII):

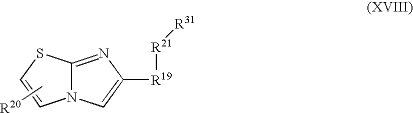

or a salt thereof, wherein

R¹⁹ is selected from:

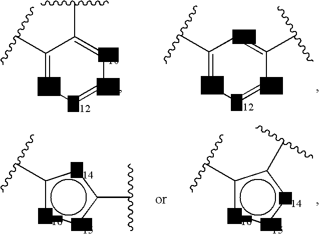

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

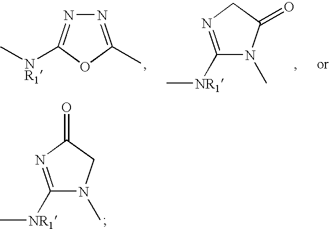

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

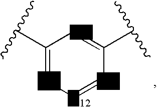

Z₁₀, Z₁₁, and Z₁₃ are each CH, R²⁰ is H, and R²¹ is —NHC(O)—, R³¹ is not an optionally substituted phenyl.

In certain embodiments, R¹⁹ is selected from:

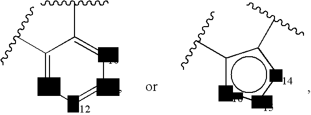

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′—R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′R₁′—, —NR₁′—S(O)₂—NR₁′—, —NR₁′—C(O)—NR₁′—S(O)₂—, NR₁′—CR₁′R₁′—C(O)—NR₁′—; —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—, —CR₁′R₁′—, —NR₁′—C(O)—O—,

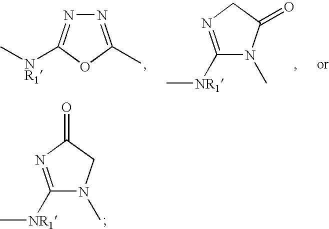

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, compounds of Structural Formula (XVIII) have the formula:

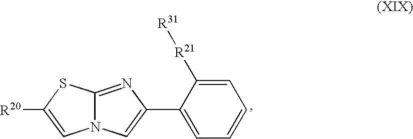

or a salt thereof; wherein

R²⁰ is selected from H or a solubilizing group;

R²¹ is selected from —NH—C(O)—, or —NH—C(O)—CH₂—; and

R³¹ is selected from an optionally substituted monocyclic or bicyclic aryl, or an optionally substituted monocyclic or bicyclic heteroaryl.

Typically, R¹⁹ in compounds of Structural Formula (XVIII) is selected from phenyl, pyridyl, thienyl or furyl, particularly optionally substituted phenyl.

Typically, R²⁰ is selected from H, —CH₂—N(CH₃)₂,

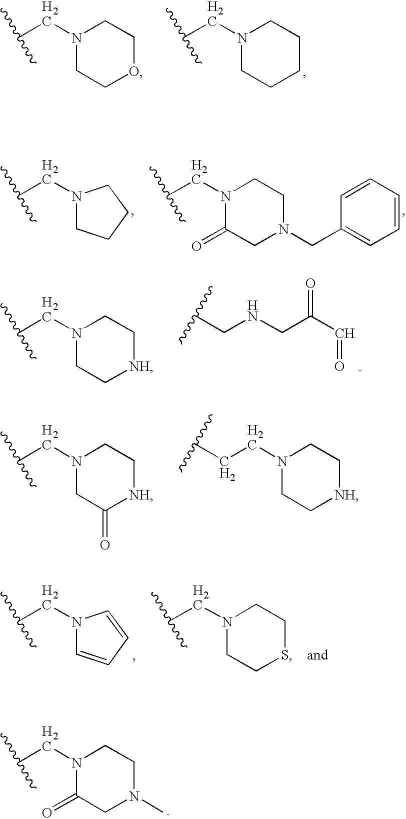

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In certain such embodiment when R²¹ is —NR₁′—C(O)—, R³¹ is not 4-cyanophenyl or

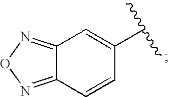

and/or when R²¹ is NR₁′—S(O)₂—, R³¹ is not 4-methoxyphenyl or t-butylphenyl.

In certain such embodiments, when R¹⁹ is

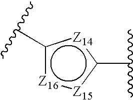

or

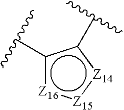

and R²¹ is NR₁′—C(O)—, R³¹ is not 4-cyanophenyl or

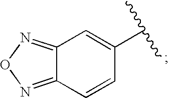

and/or when R¹⁹ is

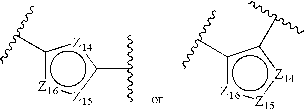

and R²¹ is —NR₁′—S(O)₂—, R³¹ is not 4-methoxyphenyl or 4-t-butylphenyl.

In another aspect, the invention provides [symbol] -modulating compounds of Structural Formula (XX):

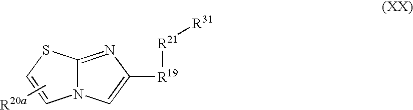

or a salt thereof, wherein

R¹⁹ is selected from:

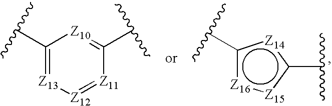

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

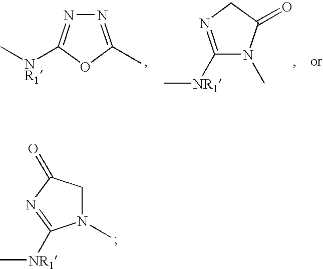

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

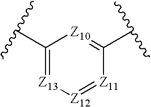

and Z₁₀, Z₁₁, Z₁₂ and Z₁₃ are each CH, R^{20a} is a solubilizing group.

Typically, R¹⁹ in compounds of Structural Formula (XX) is selected from phenyl, pyridyl, thienyl or furyl, particularly optionally substituted phenyl.

Typically, R^{20a} is selected from H, —CH₂—N(CH₃)₂,

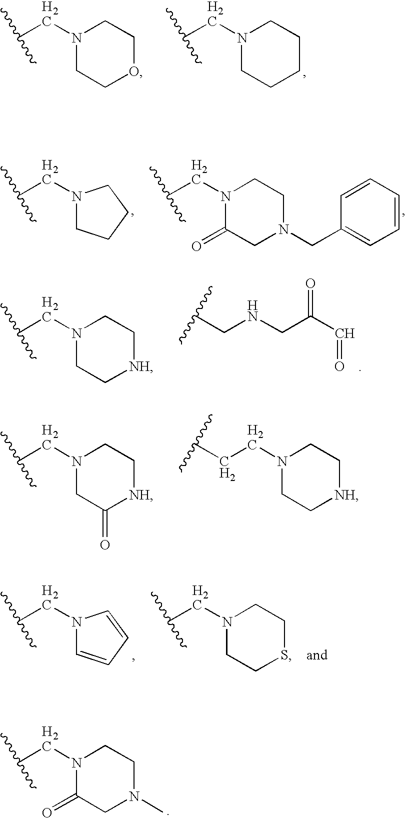

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In yet another aspect, the invention provides [symbol] -modulating compounds of Structural Formula (XXI):

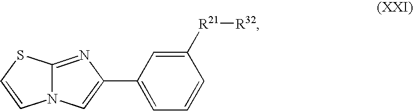

or a salt thereof wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′—R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

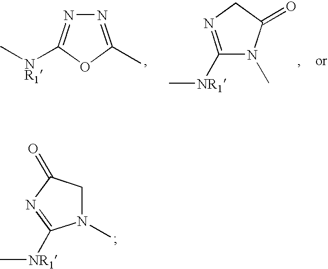

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In certain embodiments, R³² is selected from pyrrolyl, pyrazolyl, pyrazinyl, furyl, pyridyl, pyrimidinyl, or thienyl, and R³² is optionally substituted and is optionally benzofused.

In certain embodiments, R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

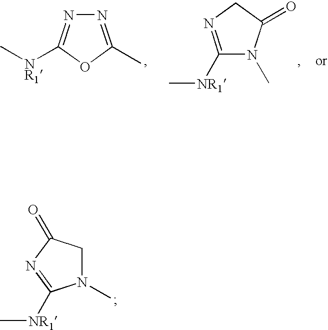

wherein
each R₁′ is independently selected from H or optionally substituted C₁-C₃ straight or branched alkyl; and

R³² is selected from benzofuryl, methylfuryl, benzothienyl, pyridyl, pyrazinyl, pyrimidinyl, pyrazolyl, wherein said methylfuryl, pyridyl, pyrazinyl, pyrimidinyl or pyrazolyl is optionally benzofused and wherein R³² is optionally substituted or further substituted.

In a further aspect, the invention provides [symbol] -modulating compounds of Structural Formula (XXII):

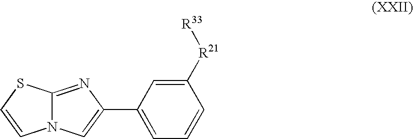

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—CO)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—C(O)—CR₁′R₁′—O—, —NR₁′—S(O)₂—CR₁′R₁′—, —NR₁′—S(O)₂—CR₁′R₁′—CR₁′R₁′—,

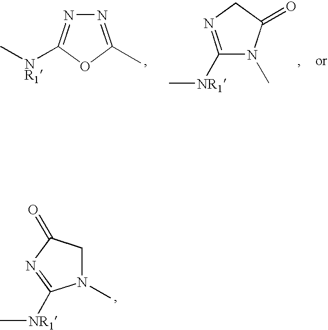

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

Preferably, R²¹ is selected from —NH—C(O)— or —NH—C(O)—CH₂—.

In one aspect, the invention provides [symbol] -modulating compounds of Structural Formula (XXII):

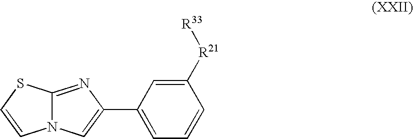

or a salt thereof, wherein:

R²¹ is selected from —NH—C(O)—, or —NH—C(O)—CH₂—; and

R³³ is phenyl substituted with

e) one —N(CH₃)₂ group;

f) one CN group at the 3 position;

g) one —S(CH₃) group; or

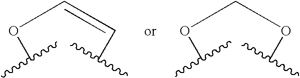

bridging the 3 and 4 positions.

In another aspect, the invention provides [symbol] -modulating compounds of Structural Formula (XXII):

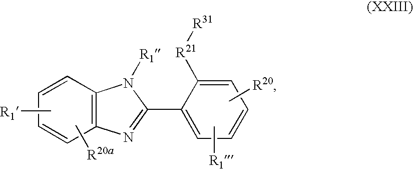

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁═CR₁′—, —NR₁′—S(O)₂—NR₁′—, —NR₁′—C(O)—NR₁′—S(O)₂—, —NR₁′R₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl,

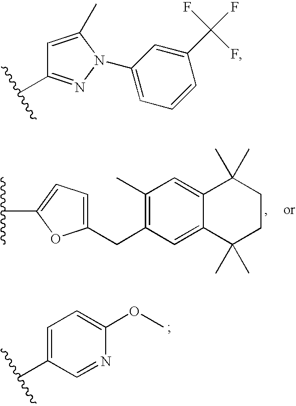

when R²¹ is —NH—C(O)— and each of R²⁰, R^{20a}, R₁′, R₁″, and R₁′″ is hydrogen, R³¹ is not

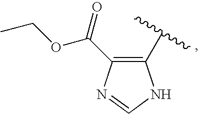

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;
when R²¹ is —NH—C(O)—, R₁″ is methyl; and each of R²⁰, R^{20a}, R₁′ and R₁′″ is hydrogen, R³¹ is not 2-methylaminophenyl,

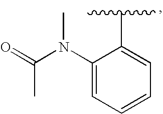

or

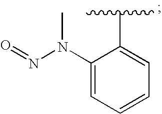

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;
when R²¹ is —NH—S(O)₂—, R₁″ is hydrogen or methyl, and each of R², R^{20a}, R₁″ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

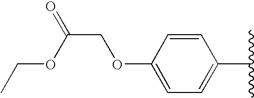

or

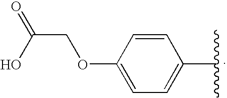

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3-substituents independently selected from —OCH₃, —N(CH₃)₂, or a solubilizing group. Suitable examples of R³ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In a particular aspect, the invention provides [symbol] -modulating compounds of Structural Formula (XXIII):

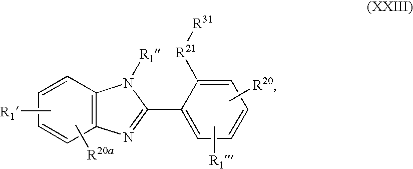

or a sat thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl,
wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In certain embodiments, R²¹ is selected from —NH—C(O)—, or —NH—C(O)—NR₁—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In yet another aspect, the invention provides [symbol] -modulating compounds of Structural Formula (XXIV):

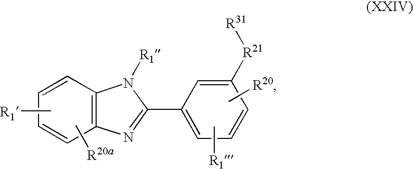

or a salt thereof wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and R³¹ is selected from an optionally substituted monocyclic or bicyclic aryl, or an optionally substituted monocyclic or bicyclic heteroaryl, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;
when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

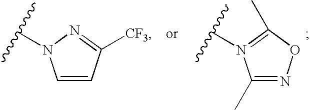

when R²¹ is —NH—C(O)—CH₂—, R₁′ is methyl, and each of R²⁰, R^{20a}, R₁″, and R₁′″ is hydrogen, R³¹ is not unsubstituted phenyl;
when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;
when R²¹ is —NH—C(O)—CH(CH₂CH₃)—, and each of R²⁰, R^{20a}, R₁′, R₁″, and R₁′″ is hydrogen, R³¹ is not unsubstituted phenyl;
when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] -modulating compounds of Structural Formula (XXIV):

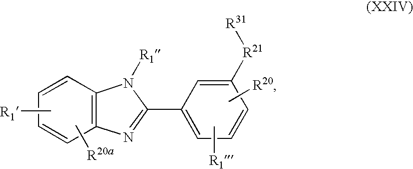

or a salt thereof, wherein:
each R²⁰ and R^{20a} is independently selected from H or a solubilizing group and at least one of R²⁰ and R^{20a} is a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)ᵣ—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and
R³¹ is selected from an optionally substituted monocyclic or bicyclic aryl, or an optionally substituted monocyclic or bicyclic heteroaryl.

In certain embodiments, when R²¹ is —NH—C(O)—CH₂—, R³¹ is not 2-methylphenyl; or 3,4-dimethoxyphenyl; when R²¹ is —NH—C(O)—CH═CH—, R³¹ is not 2-chlorophenyl; and/or when R²¹ is —NH—C(O)—NH—, R³¹ is not unsubstituted benzimidazolyl.

In a further aspect, the invention provides [symbol] -modulating compounds of Structural Formula (XXV):

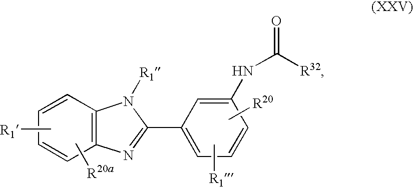

or a salt thereof, wherein:
each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In certain embodiments, R³² is selected from 3,4-dimethoxyphenyl, 2,6-dimethoxyphenyl, or 2,4-dimethoxyphenyl; wherein R³² is further optionally substituted with a solubilizing group.

In certain embodiments, R³² is not unsubstituted thienyl; unsubstituted phenyl; 2-methylphenyl; 4-fluorophenyl; 4-methoxyphenyl; 4-methylphenyl; 3,4-dioxyethylenephenyl; 3-acetylamino-4-methylphenyl; 3-[(6-amino-1-oxohexyl)amino]-4-methylphenyl; 3-amino-4-methylphenyl; 3,5-dimethoxyphenyl; 3-halo-4-methoxyphenyl; 3-nitro-4-methylphenyl; or 4-propoxyphenyl.

In one aspect, the invention provides [symbol] -modulating compounds of Structural Formula (XXIV):

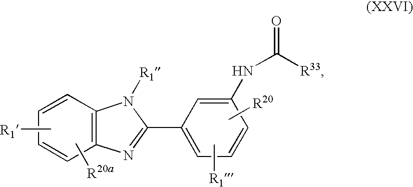

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

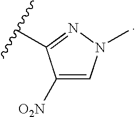

In a particular aspect, the invention provides [symbol] -modulating compounds of Structural Formula (XXVI):

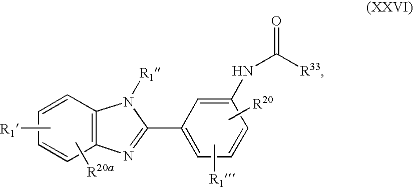

or a salt thereof, wherein:
each R²⁰ and R^{20a} is independently selected from H or a solubilizing group, wherein at least one of R²⁰ or R^{20a} is a solubilizing group;
each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] -modulating compounds of Structural Formula (XXVII):

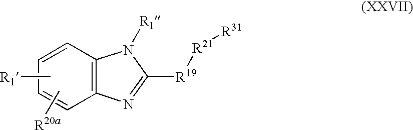

wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

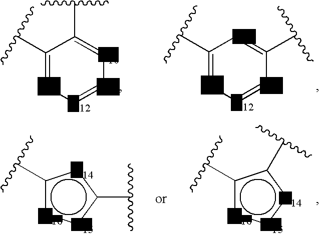

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

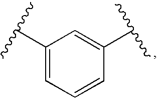

R³¹ is not unsubstituted pyridyl, 2,6-dimethoxyphenyl, 3,4,5-trimethoxyphenyl or unsubstituted furyl.

In a particular aspect, the invention provides [symbol] -modulating compounds of Structural Formula (XXVII):

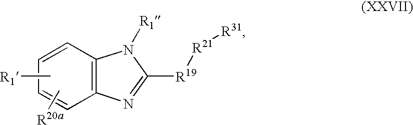

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

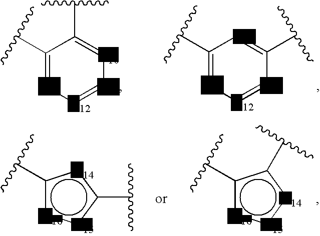

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O,
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or —O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O), —NR₁′—S(O)₂, —NR₁′—C(O)—NR₁′—, NR₁′—CS)—NR₁′—NR₁′—C(S)—NR₁′—CR₁′R₁′—, —NR₁′(O)—CR₁′R₁′—NR₁′—, —NR₁—C(═NR₁′)—NR₁′, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′—R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—, and R¹⁹ is thiazolyl, R³¹ is not optionally substituted phenyl or optionally substituted pyridyl;
when R²¹ is —NH—C(O)—CH₂—, and R¹⁹ is pyrazolyl, R³¹ is not unsubstituted indolyl or unsubstituted phenyl;
when R²¹ is —NH—C(O)—CH₂—, and R¹⁹ is

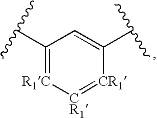

R³¹ is not 2-methylphenyl or 3,4-dimethoxyphenyl;
when R²¹ is —NH—C(O)—CH═CH—, and R¹⁹ is

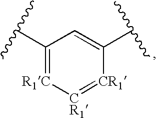

R³¹ is not 2-chlorophenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;
when R²¹ is —NH—C(O)—NH—, and R¹⁹ is

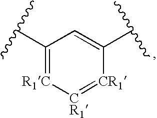

R³¹ is not unsubstituted benzimidazolyl;
when R²¹ is —NH—, and R¹⁹ is pyrazolyl, R³¹ is not unsubstituted pyridyl;
when R²⁰ is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;
when R²¹ is —NH— and R¹⁹ is pyridyl, oxadiazolyl or thiadiazolyl, R³¹ is not unsubstituted phenyl, 3-methoxyphenyl or 4-methoxyphenyl;
when R²¹ is —NH—C(O)— and R¹⁹ is thiazolyl or pyrimidinyl, R³ is not unsubstituted phenyl;
when R²¹ is —NH—C(O)— and R¹⁹ is

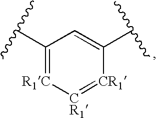

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;
when R²¹ is —NH—C(O)— and R¹⁹ is

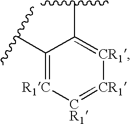

R³¹ is not 3,5-dinitrophenyl, 4-butoxyphenyl,

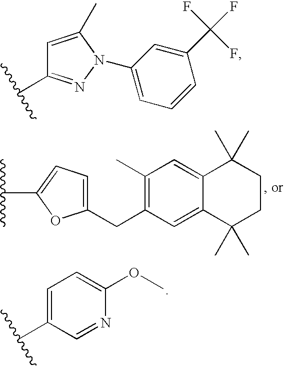

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In certain embodiments, when R²¹ is —NH—C(O)—, R¹⁹ is not

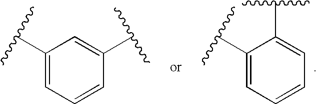

In certain embodiments, when R²¹ is —NH—C(O)—, R¹⁹ is not optionally substituted pyrazolyl, thiazolyl, thienyl, pyrrolyl or pyrimidinyl; when R²¹ is —NH—C(O)—CH2— or —NH—C(O)—NH—, R¹⁹ is not pyrazolyl; and/or when R²¹ is —NH—, R¹⁹ is not optionally substituted pyridyl, thiazolyl, pyrazolyl, thiadiazolyl, or oxadiazolyl.

In a more particular aspect, the invention provides [symbol] -modulating compounds of Structural Formula (XXVII):

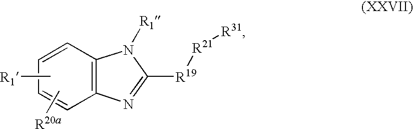

or a salt thereof, wherein:
each R²⁰ and R^{20a} is independently selected from H or a solubilizing group;

each R₁′ and R₁″ is independently selected from H or optionally substituted C₁-C₃ straight or branched alkyl;

R¹⁹ is selected from:

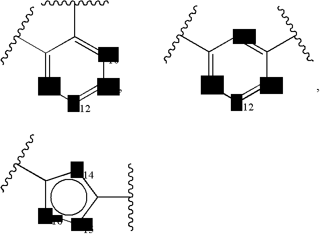

or

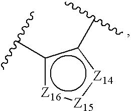

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
one to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—, —C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and
when R²¹ is —NH—C(O)— and R¹⁹ is thiazolyl or pyrimidinyl, R³¹ is not unsubstituted phenyl.

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In yet another aspect, the invention provides compounds of Structural Formula (XXVIII):

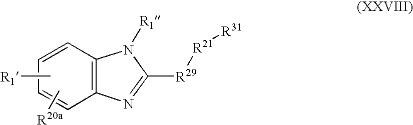

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R²⁹ is selected from:

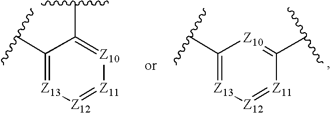

wherein:

each Z₁₀, Z₁₂ an Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′—CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

In certain embodiments, R³¹ is optionally substituted phenyl, such as 3-methoxyphenyl, 3,4-dimethoxyphenyl, 3,4,5-trimethoxyphenyl, or 4-dimethylaminophenyl.

In certain embodiments, R²¹ is —NH—C(O)—.

In preferred embodiments, R²¹ is —NH—C(O)— and R³¹ is an optionally substituted phenyl, such as 3-methoxyphenyl, 3,4-dimethoxyphenyl, 3,4,5-trimethoxyphenyl, or 4-dimethylaminophenyl.

In a further aspect, the invention provides novel [symbol] -modulating compound of Formula (VI):

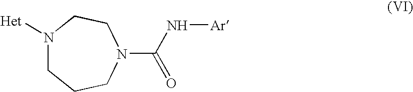

or a salt thereof, wherein:

Het is an optionally substituted heterocyclic aryl group; and

Ar′ is an optionally substituted carbocyclic or heterocyclic aryl group.

In certain embodiments, Het comprises one N heteroatom and 1 to 2 additional heteroatoms independently selected from N, O or S, such as oxazolopyridyl.

In certain embodiments, Ar′ is selected from optionally substituted phenyl, benzothiazolyl, or benzoxazolyl. When Ar′ is substituted phenyl, typically it is substituted with 1 to 3 substituents independently selected from halo, methyl, O-methyl, S-methyl or N(CH₃)₂, morpholino, or 3,4 dioxymethylene.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

In the compounds described above, bivalent groups disclosed as possible values for variables can have either orientation, provided that such orientation results in a stable molecule. Preferably, however, the left hand side of a bivalent group (e.g., —NR₁′—C(O)—) is attached to a bivalent arylene or heteroarylene group (e.g., R¹⁹) and the right hand side of a bivalent group is attached to a monovalent aryl group (e.g., R³¹).

[symbol] -modulating compounds of the invention having hydroxyl substituents, unless otherwise indicated, also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OS₃⁻ M⁺, where M⁺ is a suitable cation (preferably H⁺, NH₄⁺ or an alkali metal ion such as Na⁺ or K⁺) and sugars such as

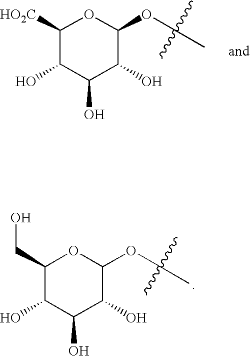

These groups are generally cleavable to —OH by hydrolysis or by metabolic (e.g., enzymatic) cleavage.

In certain embodiments, the compounds of the invention exclude one or more of the species disclosed in Tables 4-6. In certain such embodiments, the compounds of the invention exclude compound 7.

Separately or in addition to the above properties, certain [symbol] -modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity.

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A ring (e.g., 5- to 7-membered ring) or cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuryl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CRC═CR^{a}R^{b}, C═CHR^{a}—CR═CR^{a}R^{b}, —CCR^{a}, —SH, —SOₖR⁰ (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent A substituted aliphatic group can also have a non-aromatic heterocyclic ring, a substituted a non-aromatic heterocyclic ring, benzyl, substituted benzyl, aryl or substituted aryl group as a substituent A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

A hydrogen-bond donating group is a functional group having a partially positively-charged hydrogen atom (e.g., —OH, —NH₂, —SH) or a group (e.g., an ester) that metabolizes into a group capable of donating a hydrogen bond.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C6-C14) aryl, phenyl, naphthyl, (C7-C20) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)ₙ—C(O)—R^{b}, —(CH₂)—O—(CH₂)ₙ—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or R^{b} moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula: —(CH₂)ₙ—R¹⁰⁰—N(R¹⁰¹)(R¹⁰¹), wherein:

n is selected from 0, 1 or 2;
R¹⁰⁰ is selected from a bond, —C(O)—, or —O(CH₂)ₙ; and
each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;
c.

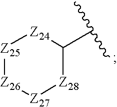

d.

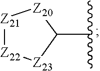

e.

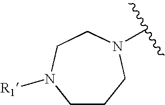

f. both R¹⁰¹ moieties are taken together with the nitrogen atom to which the are bound to form a ring of the structure

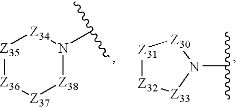

or

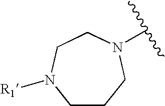

or
g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′; wherein:
each Z is independently selected from —O—, —S—, —NR₁′—, or —C(R⁵⁰)(R⁵⁰), wherein:
at least three of Z₂₀, Z₂₁, Z₂₂, and Z₂₃ are —C(R⁵⁰)(R⁵⁰)—;
at least three of Z₂₄, Z₂₅, Z₂₆, Z₂₇, and Z₂₈ are —C(R⁵⁰)(R⁵⁰)—;
at least four of Z₃₀, Z₃₁, Z₃₂, and Z₃₃ are —C(R⁵⁰)(R⁵⁰)—; and
at least four of Z₃₄, Z₃₅, Z₃₆, Z₃₇, and Z₃₈ are —C(R⁵⁰)(R⁵⁰)—;

each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;

each R⁵⁰ is independently selected from R₁′, halo, CN, OH, O—(C₁-C₄ straight or branched alkyl), N(R₁′)(R₁′), ═CR₁′, SR₁′, ═NR₁′, ═NOR₁′, or ═O;

any two suitable non-cyclic R⁵⁰ are optionally bound to one another directly or via a C₁ to C₂ alkylene, alkenylene or alkanediylidene bridge to produce a bicyclic fused or spiro ring; and

any

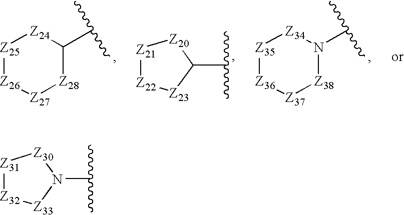

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another preferred embodiment, the solubilizing group is a moiety of the formula: —(CH₂)ₙ—O—R¹⁰¹, wherein n and R¹⁰¹ are as defined above.

In another preferred embodiment, the solubilizing group is a moiety of the formula: —(CH₂)ₙ—C(O)—R₁′, wherein n and R₁′ are as defined above.

In a more preferred embodiment, a solubilizing group is selected from —(CH₂)ₙ—R¹⁰², wherein n is 0, 1 or 2; and R¹⁰² is selected from

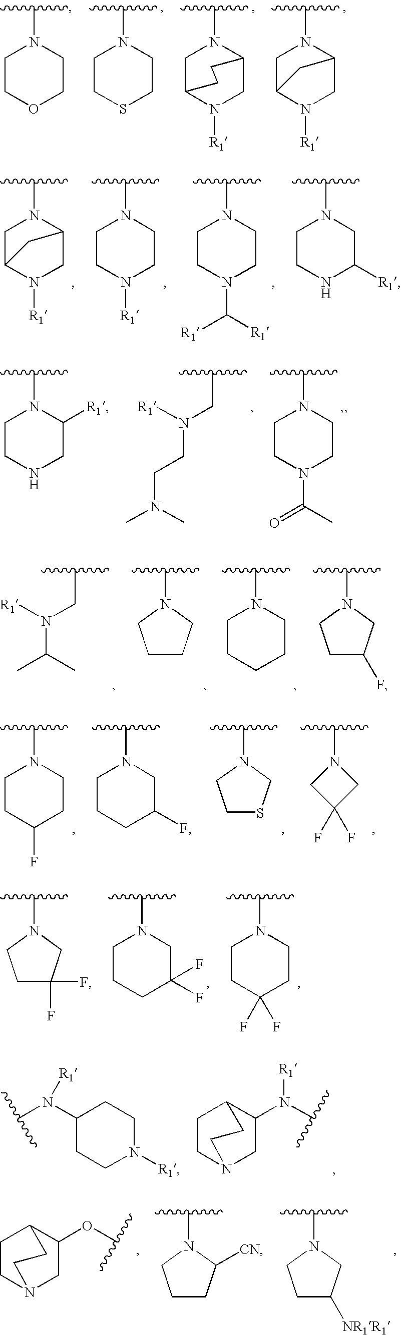

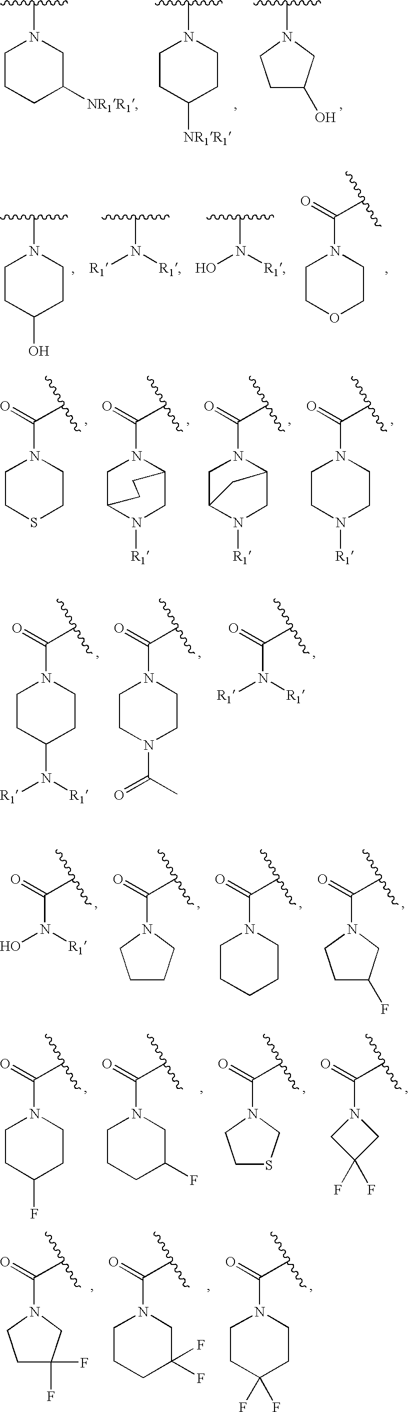

| | | | ED₅₀ | ED₅₀ | FOLD |
|---|---|---|---|---|---|
| COMPOUND | | | FP | MS | ACT. |
| NO | [M + H]+ | STRUCTURE | ASSAY | ASSAY | MS |
| 1 | | 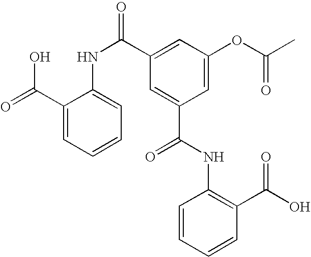 | A | NT | |
| 2 | | 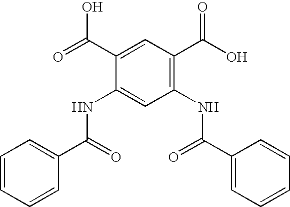 | D | NT | |
| 3 | | 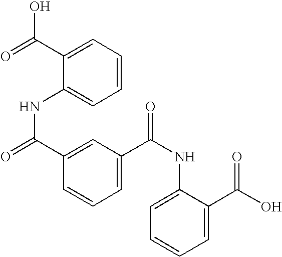 | B | NT | |
| 4 | | 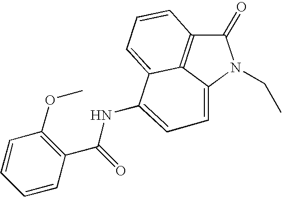 | N/A | NT | |
| 5 | | 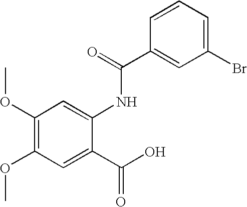 | B | NT | |
| 6 | | 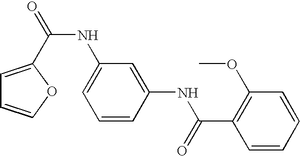 | D | NT | |
| 7 | 346 | 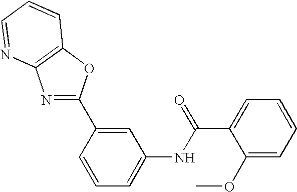 | A | NT | |
| 8 | | 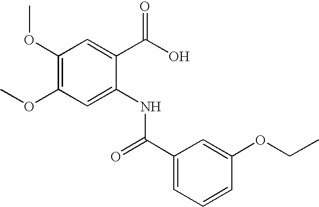 | B | NT | |
| 9 | | 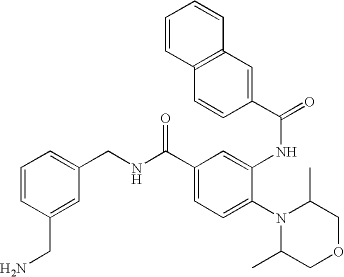 | C | NT | |
| 10 | | 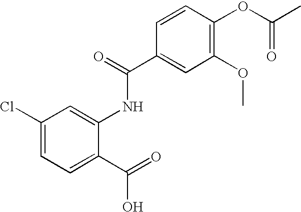 | D | NT | |
| 19 | 409.6 | 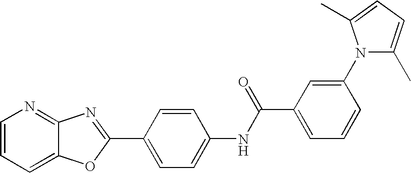 | D | D | C |
| 20 | 401.3 | 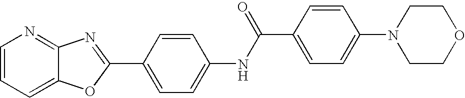 | D | D | C |
| 21 | 399.1 | 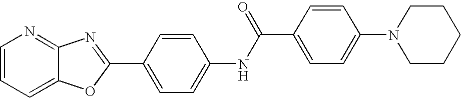 | D | D | C |
| 22 | 414.0 | 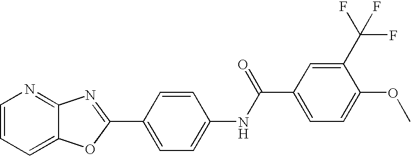 | D | D | C |
| 24 | 359.4 | 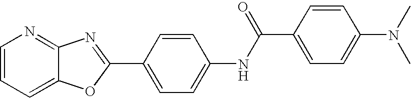 | D | D | C |
| 27 | 376.1 | 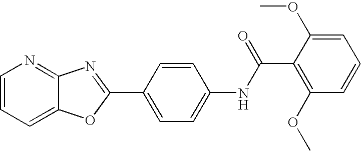 | D | D | C |
| 29 | 385.1 | 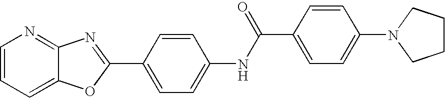 | D | D | C |
| 31 | 360.1 | 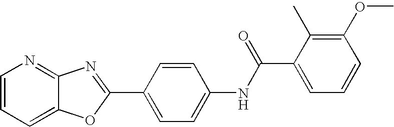 | D | D | C |
| 32 | 400.0 | 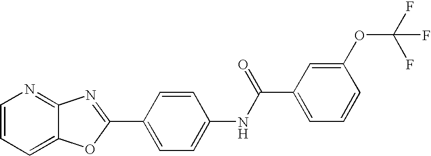 | D | D | C |
| 33 | 376.1 | 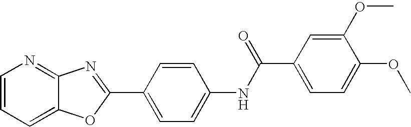 | D | D | C |
| 34 | 406.3 | 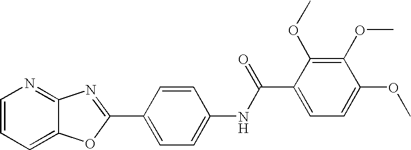 | D | D | C |
| 35 | 346.5 | 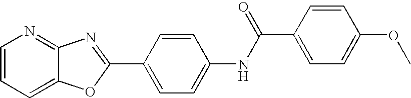 | D | D | C |
| 36 | 376.7 | 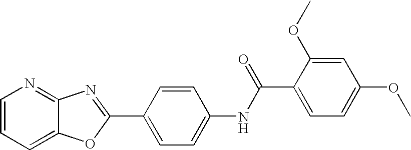 | D | D | C |
| 37 | 316.4 | 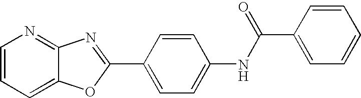 | D | D | C |
| 38 | 401.0 | 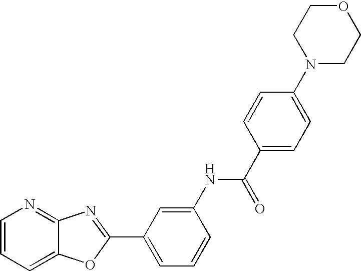 | D | D | C |
| 39 | 399.1 | 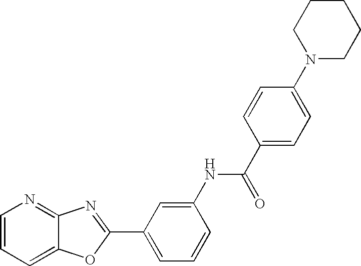 | D | D | C |
| 40 | 414.2 | 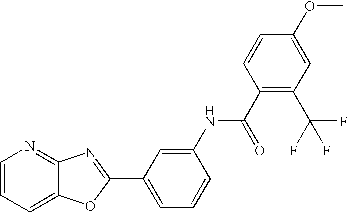 | D | D | C |
| 41 | 414.2 | 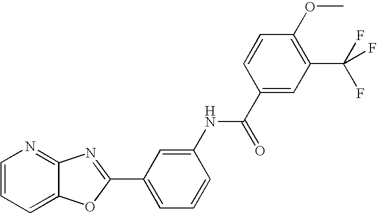 | D | D | C |
| 42 | 359.1 | 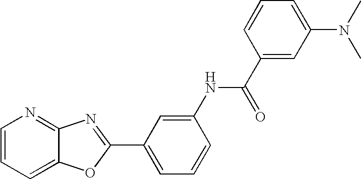 | A | A | B |
| 43 | 359.1 | 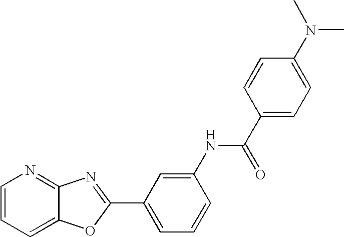 | A | A | B |
| 45 | 341.0 | 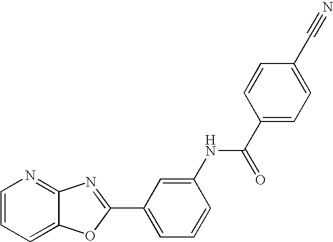 | D | D | C |
| 46 | 376.1 | 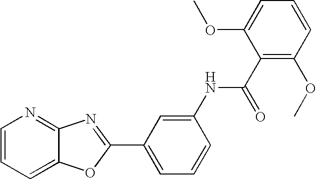 | D | D | C |
| 48 | 406.3 | 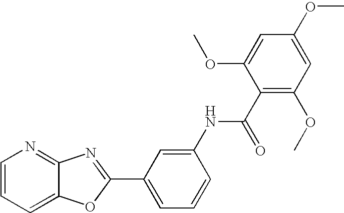 | D | D | C |
| 49 | 360.1 | 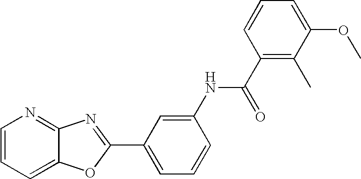 | D | A | B |
| 50 | 400.0 | 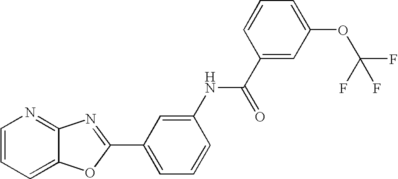 | NT | D | |
| 51 | 360.1 | 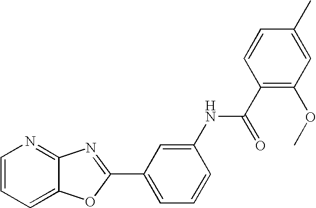 | A | A | B |
| 52 | 376.1 | 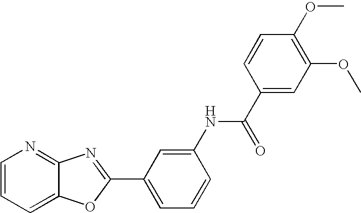 | A | A | B |
| 53 | 406.1 | 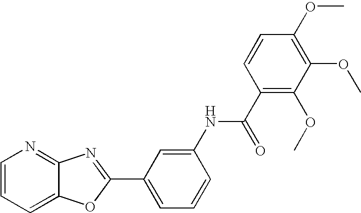 | D | D | C |
| 54 | 346.4 | 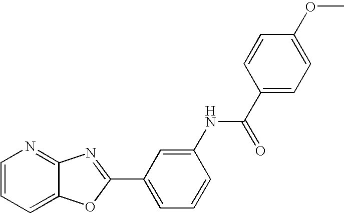 | NT | D | |
| 55 | 376.1 | 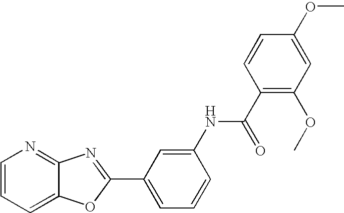 | A | A | B |
| 56 | 316.0 | 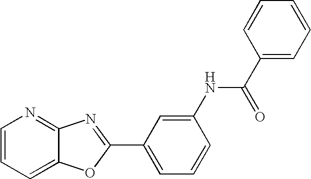 | D | A | B |
| 57 | 407.1 | 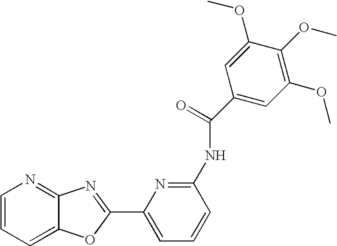 | | A | |
| 58 | 377.1 | 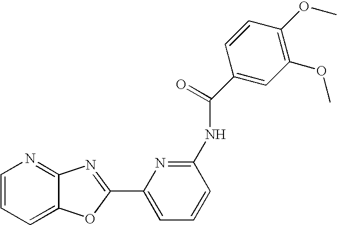 | | NA | |
| 59 | 318.1 | 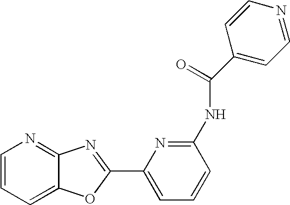 | | NA | |
| 60 | 413.1 | 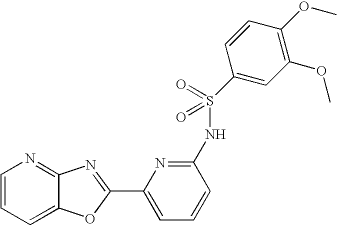 | | A | |
| 61 | 413.1 | 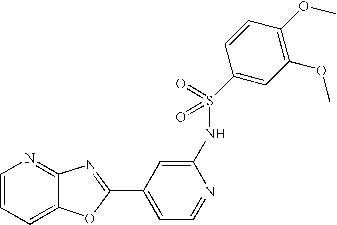 | | NA | |
| 62 | 349.0 | 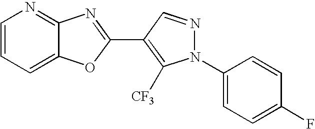 | | NA | |
| 63 | 377.1 | 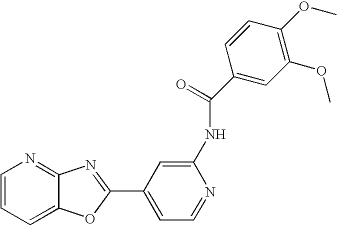 | | A | |
| 64 | 360.1 | 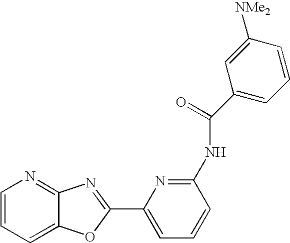 | | A | |
| 65 | 383.0 | 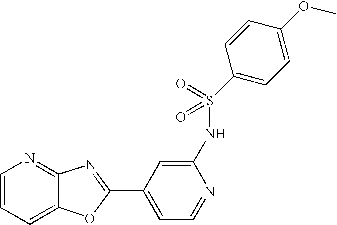 | | NA | |
| 66 | 407 | 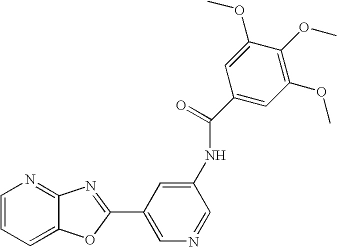 | | A | |
| 67 | 377 | 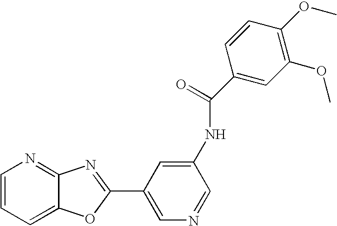 | | A | |
| 68 | 360 | 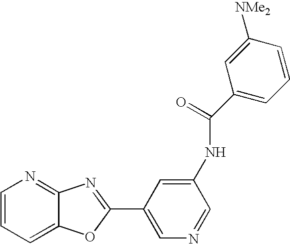 | | A | |
| 69 | 377.1 | 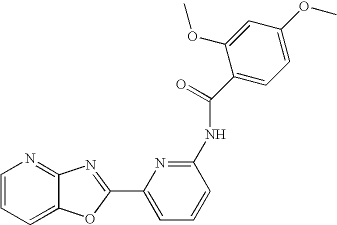 | | A | B |
| 70 | 360.1 | 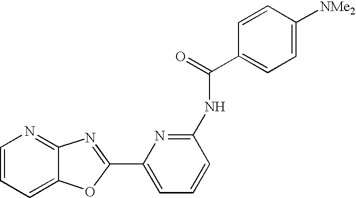 | | A | B |
| 71 | 376.1 | 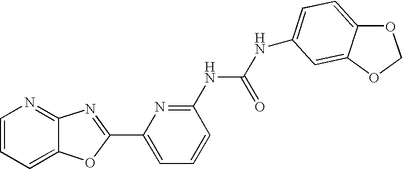 | | A | B |
| 72 | 422.1 | 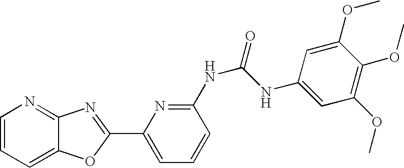 | | NT | |
| 73 | 377 | 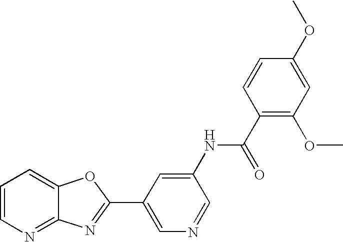 | | NT | |
| 74 | 412 | 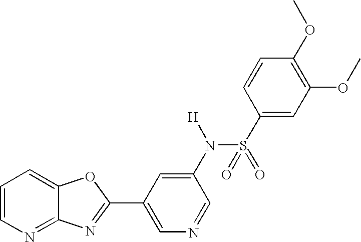 | | D | C |
| 75 | 407.1 | 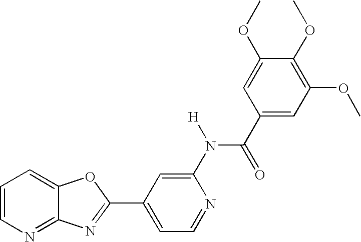 | | A | B |
| 76 | 360.1 | 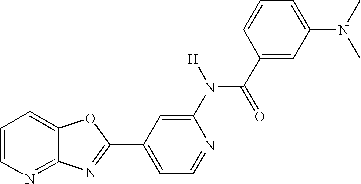 | | A | B |
| 77 | 376.1 | 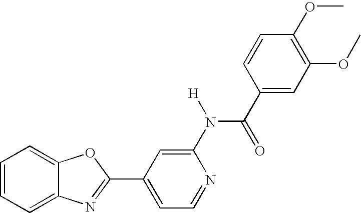 | | A | B |
| 78 | 445.1 | 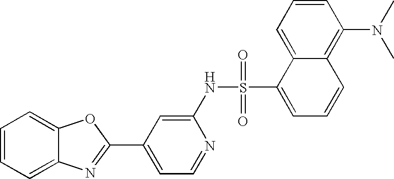 | | | |
| 79 | 338 | 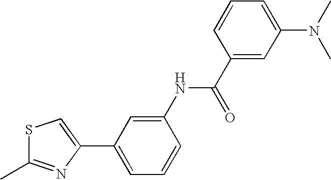 | | D | C |
| 80 | 355 | 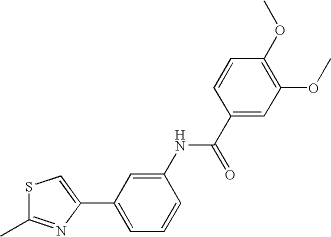 | | A | B |
| 81 | 354.5 | 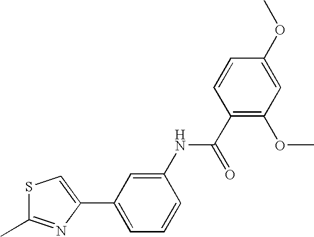 | | A | B |
| 82 | 402 | 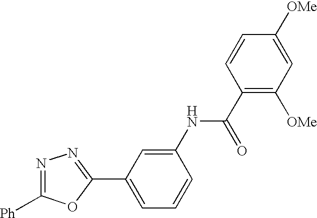 | | C | |
| 83 | 355 | 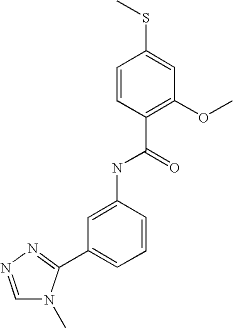 | | A | B |
| 84 | 417 | 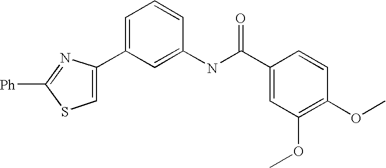 | | A | B |
| 85 | 335.1 | 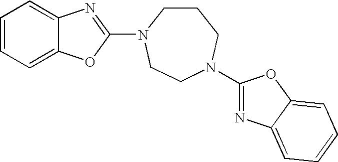 | | D | C |
| 86 | 375.1 | 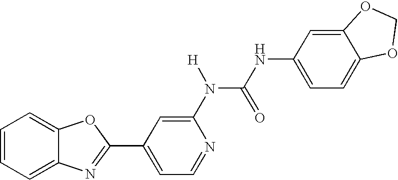 | | D | C |
| 87 | 375.1 | 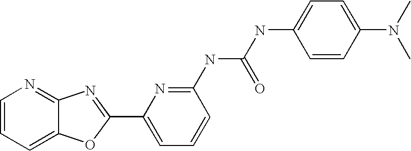 | | D | C |
| 88 | 382.1 | 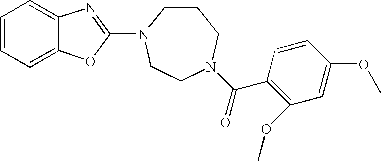 | | D | C |
| 89 | 365.1 | 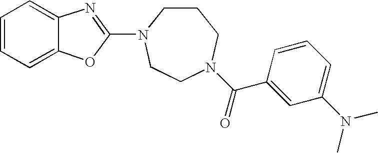 | | D | C |
| 90 | 381.1 | 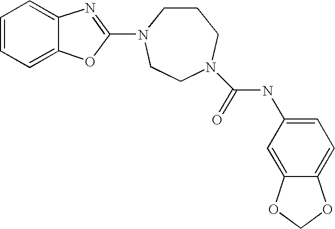 | | | |
| 91 | 412.1 | 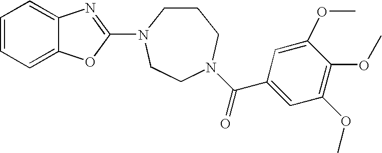 | | D | C |
| 92 | 308.1 | 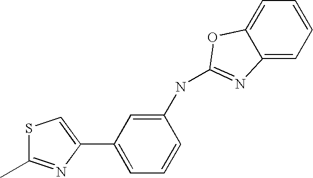 | | A | B |
| 93 | 329.1 | 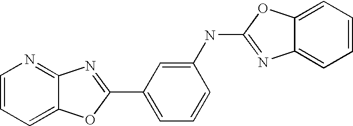 | | A | B |
| 94 | 434.1 | 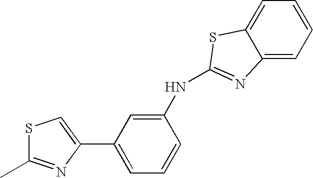 | | A | B |
| 95 | 345.1 | 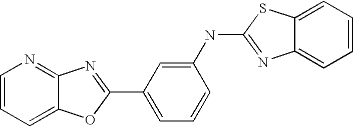 | | A′ | B |
| 96 | 376.1 | 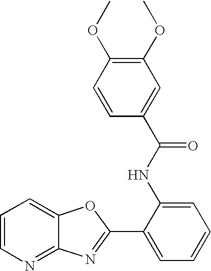 | | A′ | B |
| 97 | 359.1 | 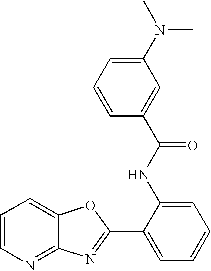 | | A′ | B |
| 98 | 408.1 | 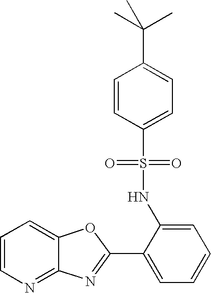 | | D | C |
| 99 | 391 | 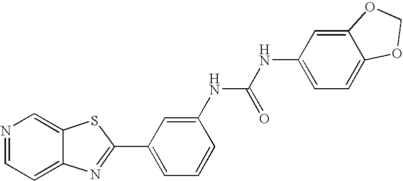 | | A′ | A |
| 100 | 376 | 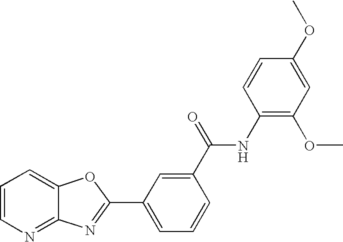 | | A | B |
| 101 | 376 | 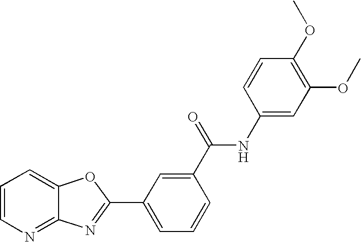 | | A | A |
| 102 | 406 | 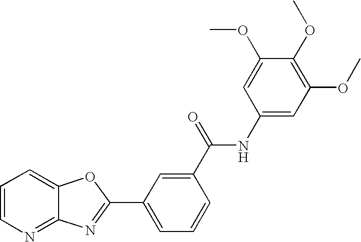 | | A | A |
| 103 | 374 | 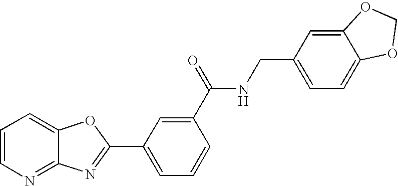 | | D | C |
| 104 | 346.1 | 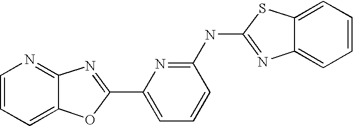 | | A′ | B |
| 105 | 330.1 | 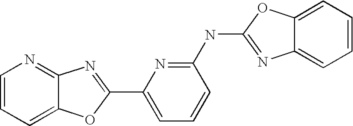 | | A′ | B |
| 106 | 406.1 | 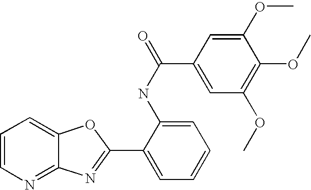 | | A′ | B |
| 107 | 488 | 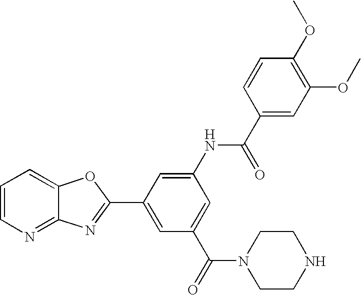 | | A | B |
| 108 | 324 | 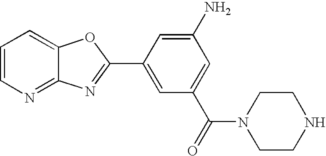 | | NA | |
| 109 | 401 | 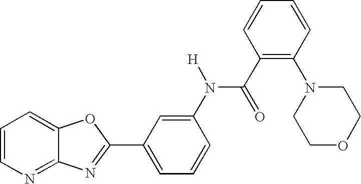 | | A | B |
| 110 | 381 | 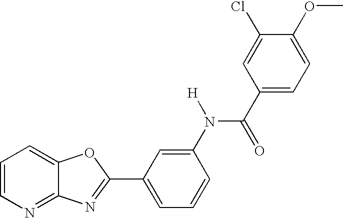 | | C | |
| 111 | 359 | 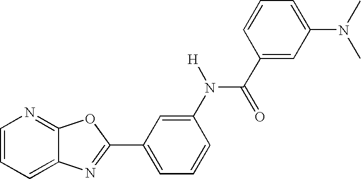 | | A | B |
| 112 | 376 | 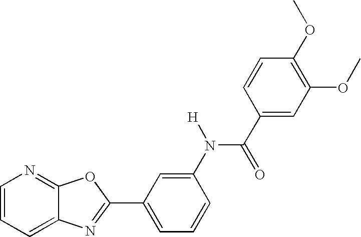 | | A | B |
| 113 | 375 | 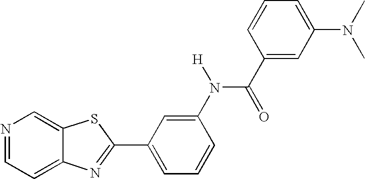 | | A | B |
| 114 | 392 | 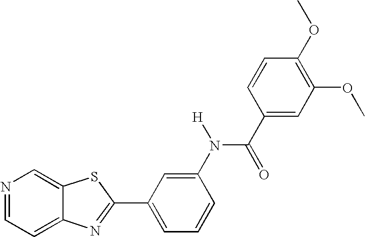 | | A′ | A |
| 115 | 422 | 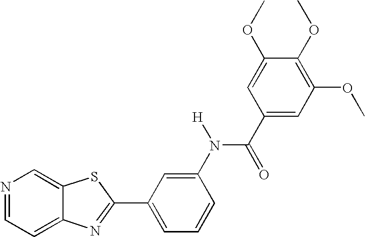 | | A | A |
| 116 | 386 | 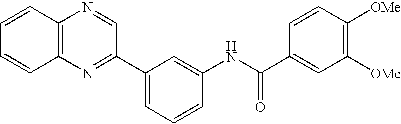 | | C | |
| 117 | 388 | 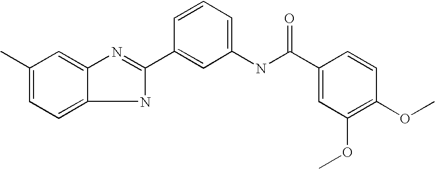 | | A | A |
| 118 | 410 | 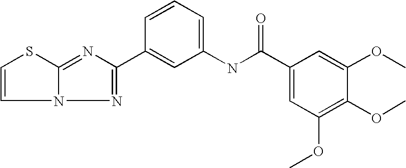 | | A | A |
| 119 | 375 | 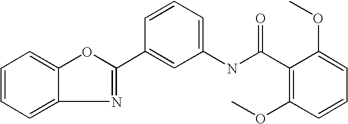 | | A | B |
| 120 | 391 | 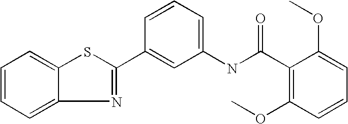 | | NT | |
| 121 | 414 | 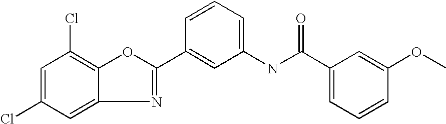 | | D | C |
| 122 | 417 | 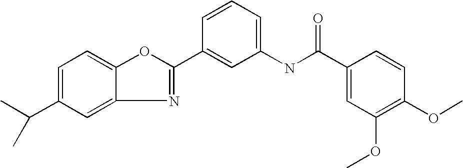 | | C | |
| 123 | 474 | 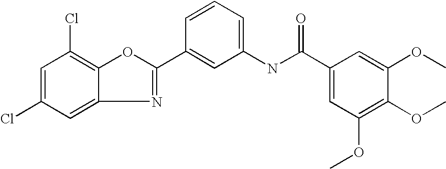 | | NT | |
| 124 | 391 | 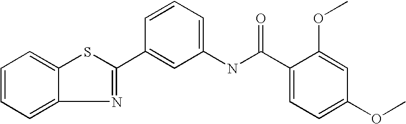 | | A | B |
| 125 | 433 | 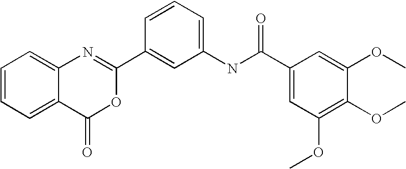 | | B | B |
| 126 | 374 | 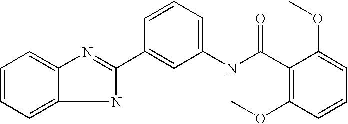 | | A | B |
| 127 | 355 | 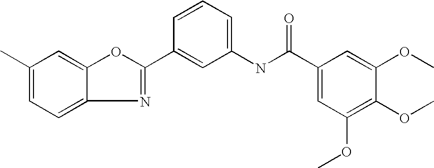 | | A | A |
| 128 | 388 | 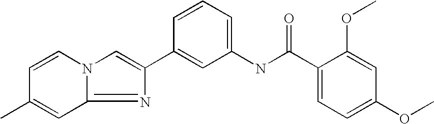 | | A | B |
| 129 | 418 | 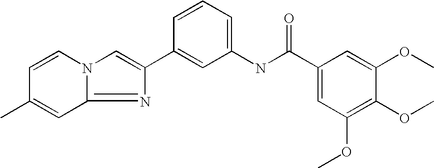 | | A′ | A |
| 130 | 358 | 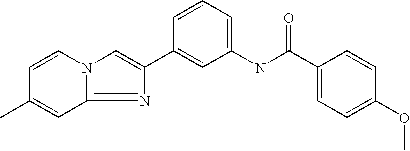 | | A | B |
| 131 | 418 | 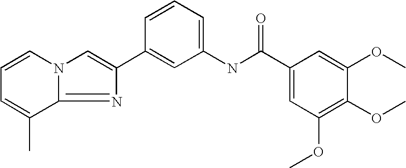 | | A | A |
| 132 | 350 | 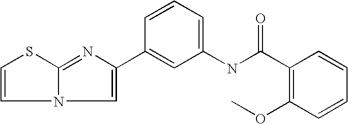 | | A | A |
| 133 | 480 | 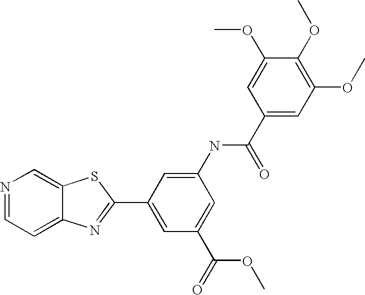 | | A′ | B |
| 134 | 466 | 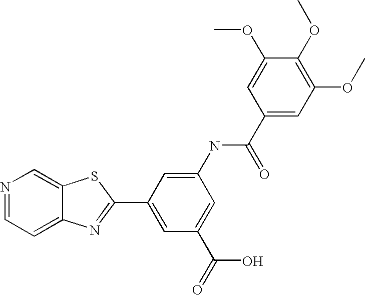 | | A′ | B |
| 135 | 452 | 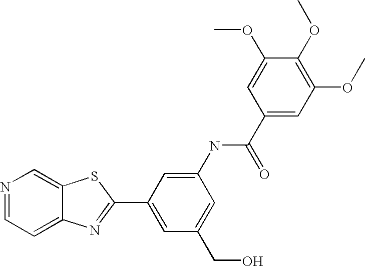 | | A′ | B |
| 136 | 434 | 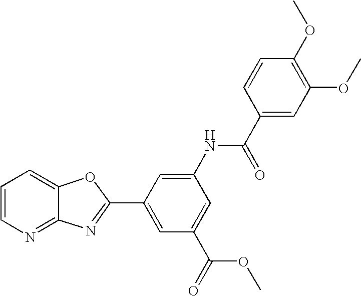 | | D | C |
| 137 | 420 | 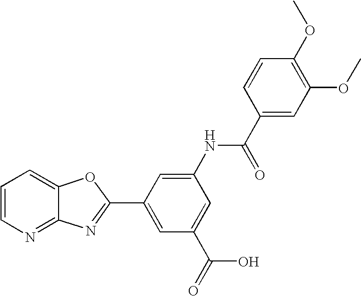 | | D | |
| 138 | 471 | 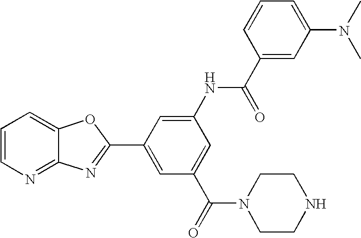 | | A | B |
| 139 | 488 | 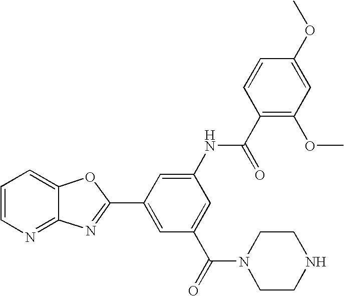 | | A | B |
| 141 | 374.1 | 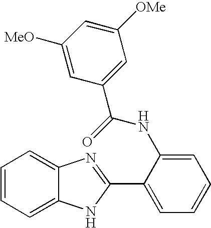 | | A′ | B |
| 142 | 374.1 | 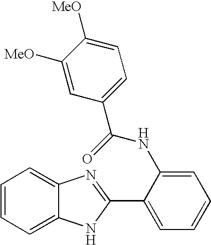 | | A′ | A |
| 143 | 410.1 | 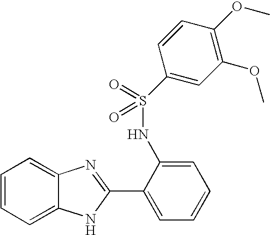 | | D | |
| 144 | 427.1 | 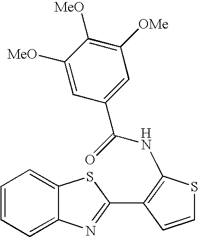 | | D | C |
| 145 | 397.1 | 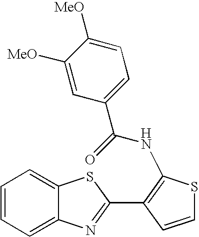 | | A′ | B |
| 146 | 397.1 | 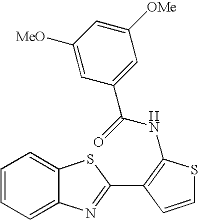 | | A′ | B |
| 147 | 392.1 | 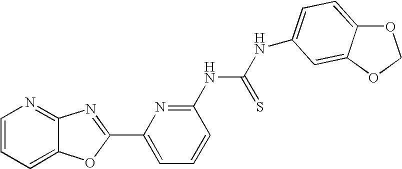 | | D | |
| 148 | 405.2 | 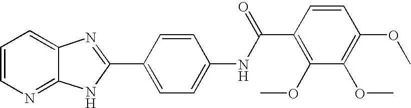 | | D | |
| 149 | 359.0 | 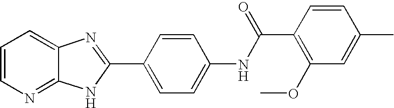 | | A′ | B |
| 150 | 375.0 | 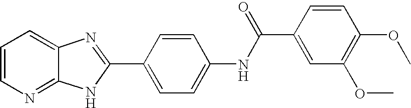 | | A′ | B |
| 151 | 375.0 | 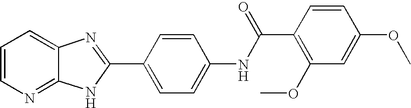 | | D | C |
| 152 | 392.1 | 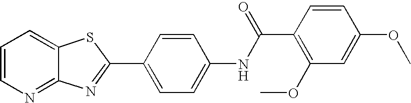 | | D | C |
| 153 | 490 | 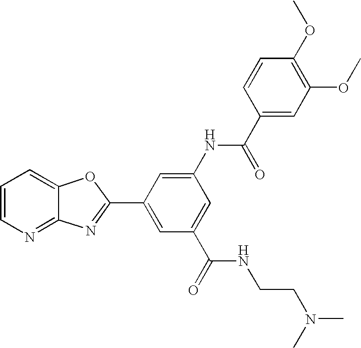 | | A | B |
| 154 | 473 | 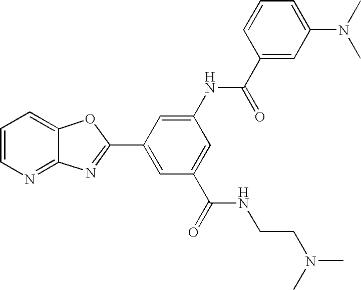 | | C | A |
| 155 | 490 | 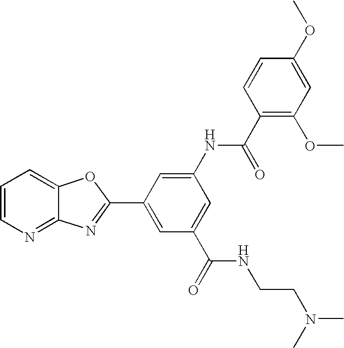 | | A | B |
| 156 | 433 | 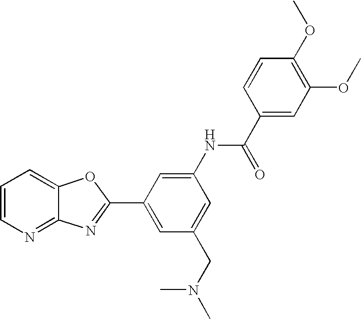 | | A | B |
| 157 | 416 | 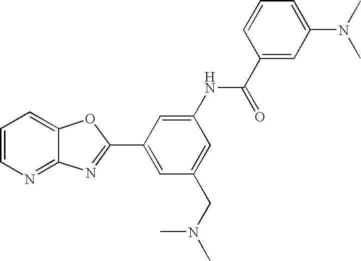 | | D | B |
| 158 | 433 | 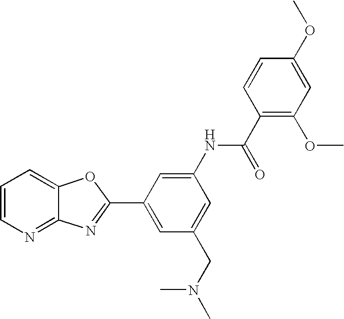 | | A | B |
| 159 | 474 | 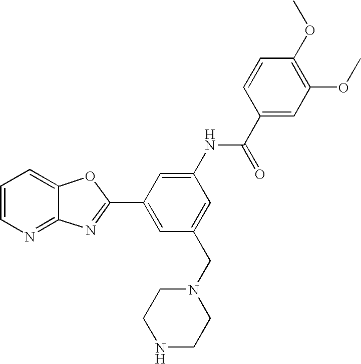 | | A | B |
| 160 | 457 | 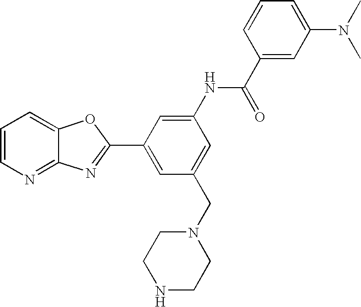 | | B | A |
| 161 | 474 | 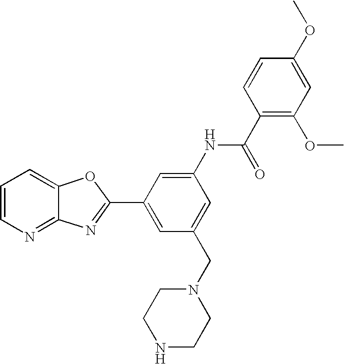 | | A | A |
| 162 | 392.1 | 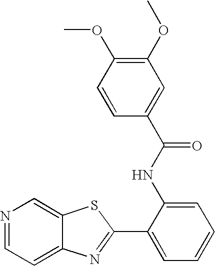 | | A′ | B |
| 163 | 422.1 | 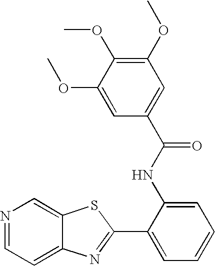 | | A′ | B |
| 164 | 488 | 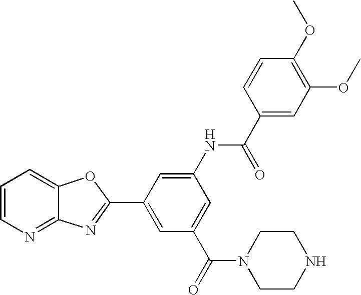 | | A | B |
| 165 | 416 | 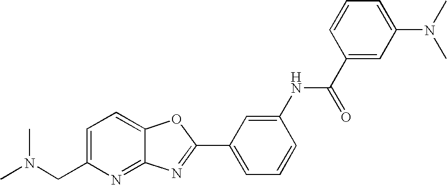 | | D | A |
| 166 | 457 | 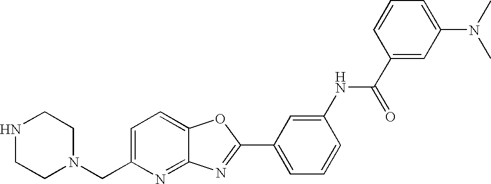 | | A | B |
| 167 | 373 | 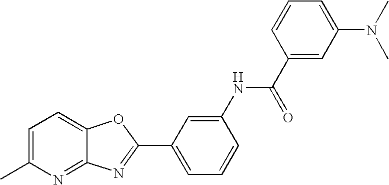 | | A | B |
| 168 | 388.1 | 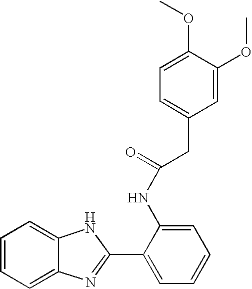 | | NA | C |
| 169 | 343.1 | 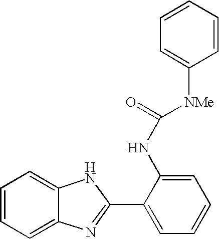 | | A′ | B |
| 174 | 479 | 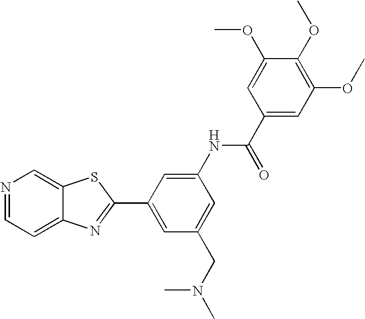 | | B | A |
| 175 | 323.1 | 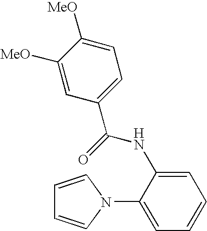 | | B | B |
| 176 | 354.1 | 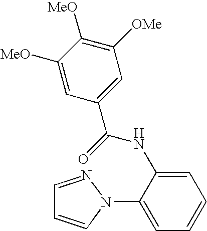 | | B | B |
| 177 | 324.1 | 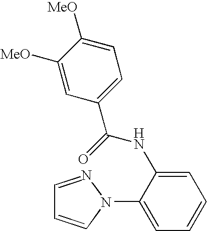 | | D | B |
| 178 | 437 | 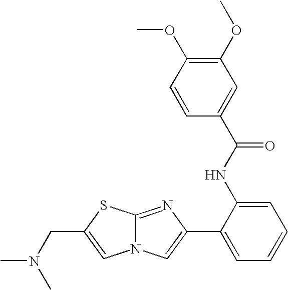 | | A | A |
| 179 | 467 | 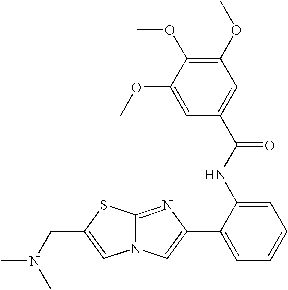 | | A′ | A |
| 180 | 358.0 | 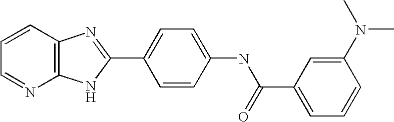 | | A | B |
| 181 | 405.0 | 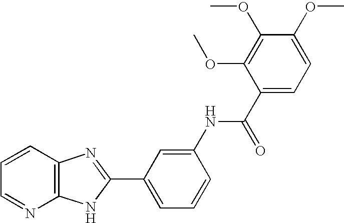 | | A′ | A |
| 182 | 359.0 | 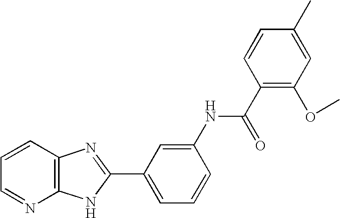 | | A′ | A |
| 183 | 358.0 | 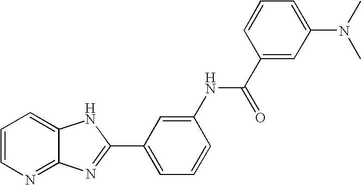 | | A | A |
| 184 | 375.0 | 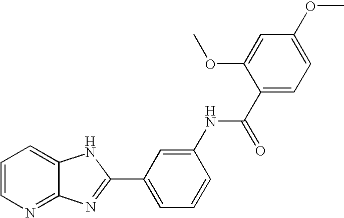 | | A′ | B |
| 185 | 374.9 | 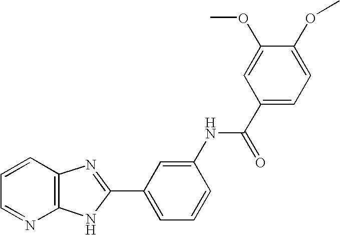 | | A′ | A |
| 186 | 405.3 | 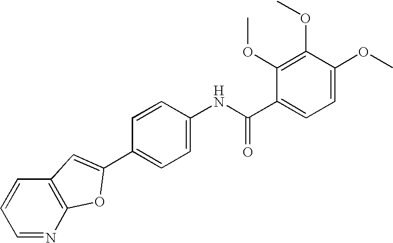 | | NA | C |
| 187 | 358.9 | 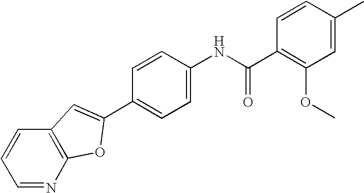 | | A | B |
| 188 | 358.9 | 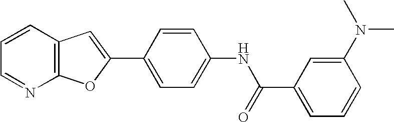 | | NA | C |
| 189 | 375.2 | 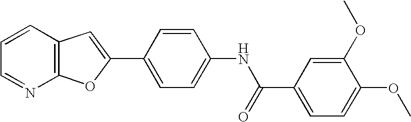 | | NA | C |
| 190 | 375.3 | 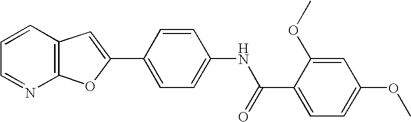 | | A′ | B |
| 191 | 375.0 | 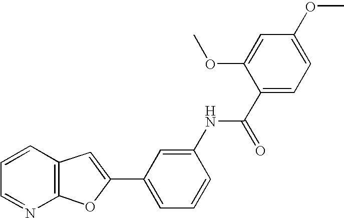 | | A′ | B |
| 192 | 375.0 | 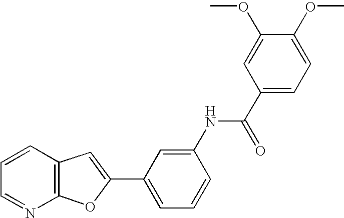 | | A | B |
| 193 | 358.0 | 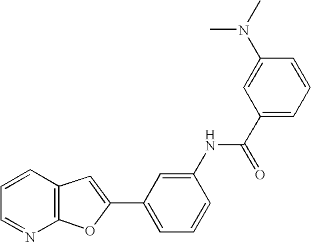 | | NA | C |
| 194 | 422.3 | 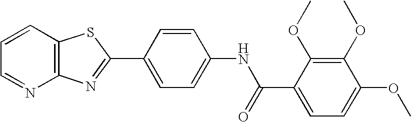 | | NA | C |
| 195 | 375.9 | 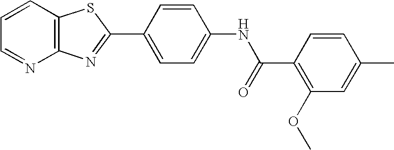 | | NA | C |
| 196 | 374.9 | 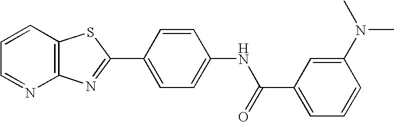 | | A | B |
| 197 | 391.1 | 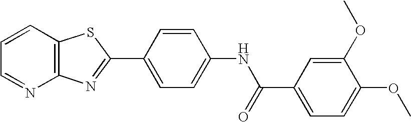 | | A′ | B |
| 198 | 422.4 | 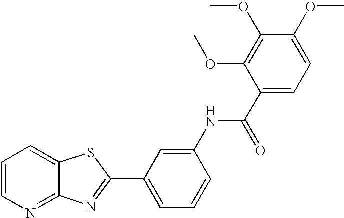 | | A | B |
| 199 | 375.9 | 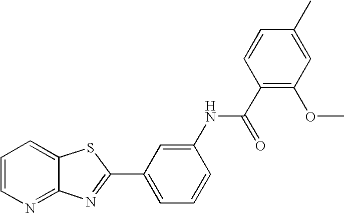 | | A′ | B |
| 200 | 375.4 | 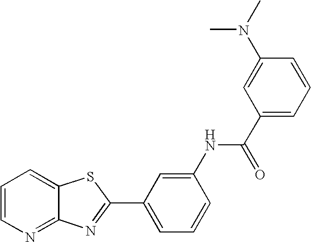 | | A′ | B |
| 201 | 391.9 | 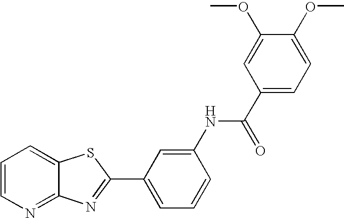 | | A | B |
| 202 | 392.4 | 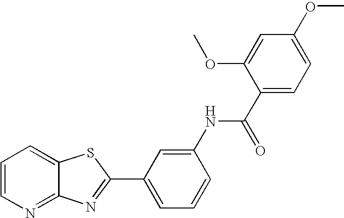 | | A′ | B |
| 203 | 380 | 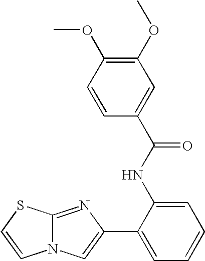 | | A′ | B |
| 204 | 410 | 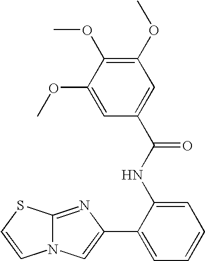 | | A′ | A |
| 205 | 437 | 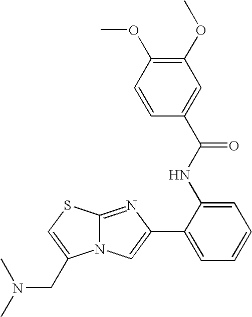 | | A | A |
| 206 | 467 | 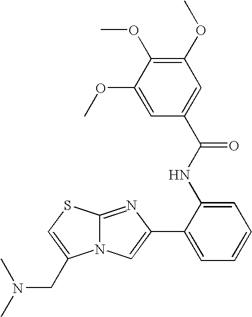 | | A | A |
| 207 | 478 | 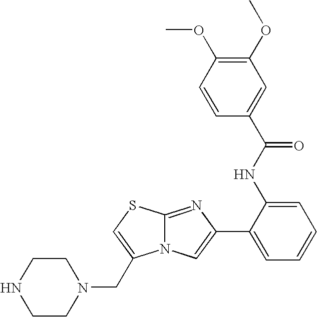 | | A | A |
| 208 | 508 | 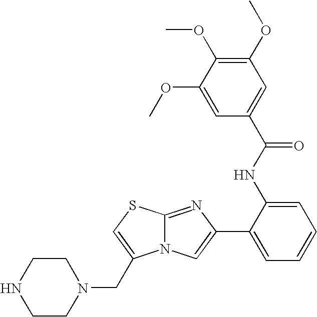 | | A | A |
| 209 | 479 | 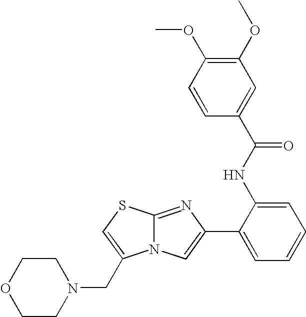 | | A | A |
| 210 | 509 | 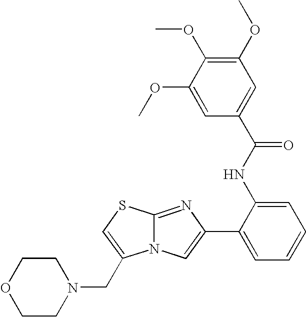 | | A | B |
| 211 | | 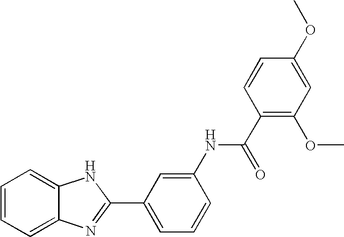 | | A′ | B |
| 212 | 362 | 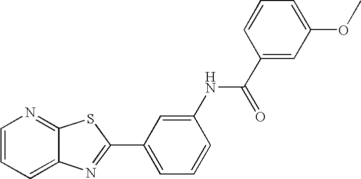 | | A | B |
| 213 | 392 | 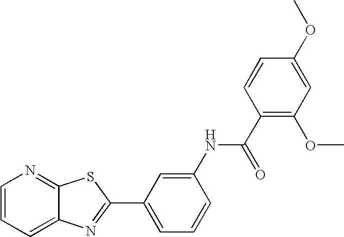 | | A′ | B |
| 214 | 392 | 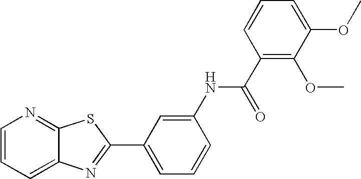 | | A | B |
| 215 | 392 | 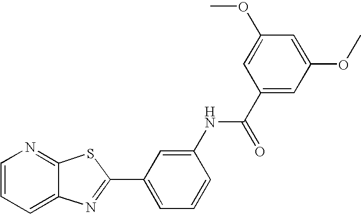 | | A′ | B |
| 216 | 362 | 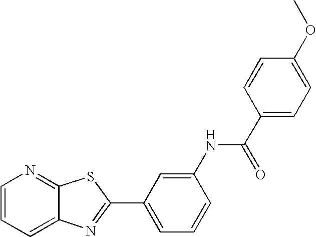 | | A′ | B |
| 217 | 422 | 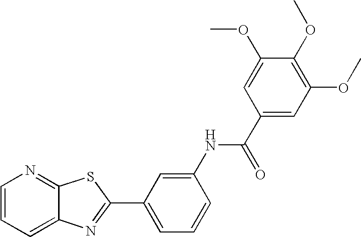 | | A′ | A |
| 218 | 405 | 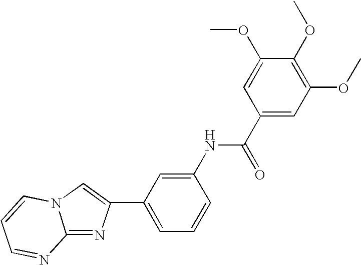 | | A′ | A |
| 219 | 419 | 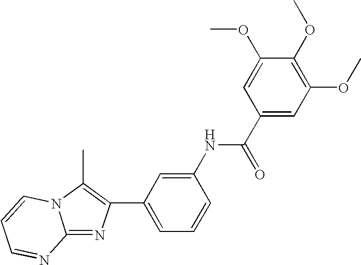 | | A | A |
| 220 | 423 | 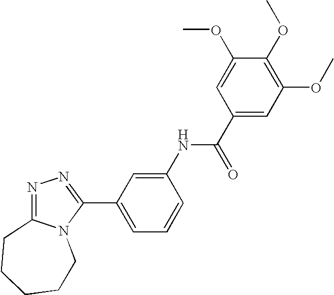 | | NA | C |
| 221 | 321.4 | 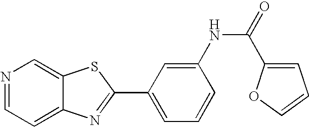 | | A | B |
| 222 | 366.8 | 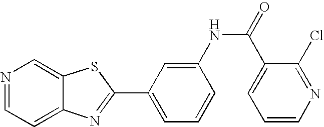 | | A | B |
| 223 | 337.4 | 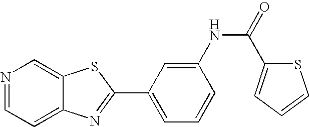 | | A′ | B |
| 225 | 332.4 | 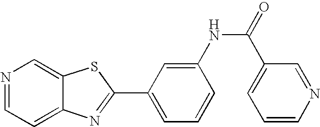 | | NA | C |
| 226 | 412.5 | 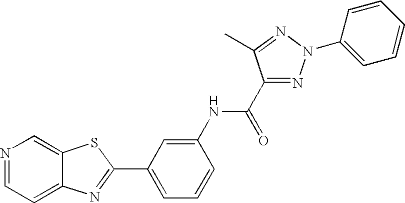 | | | |
| 227 | 333.4 | 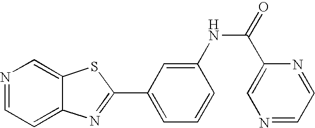 | | NA | C |
| 228 | 391.5 | 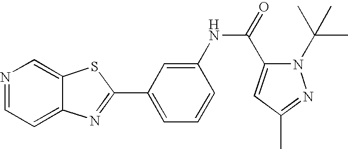 | | A | A |
| 229 | 418.5 | 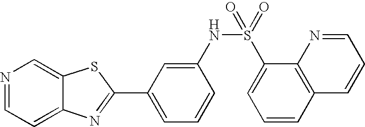 | | NA | C |
| 230 | 459.5 | 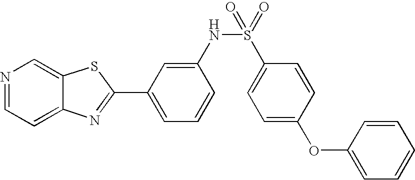 | | NA | C |
| 231 | 425.5 | 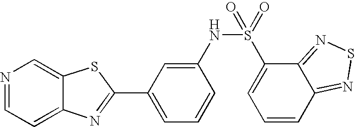 | | NA | C |
| 232 | 423.5 | 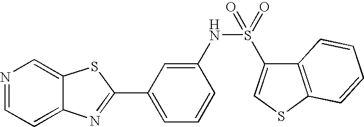 | | NA | C |
| 234 | 449.5 | 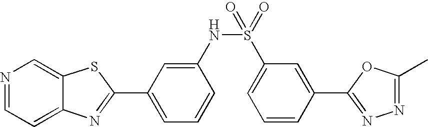 | | NA | C |
| 235 | 436.3 | 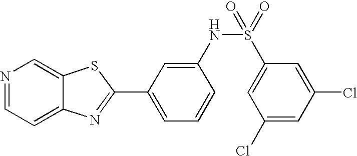 | | NA | C |
| 236 | 423.5 | 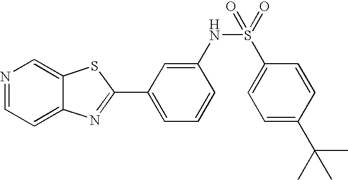 | | NA | C |
| 237 | 450.5 | 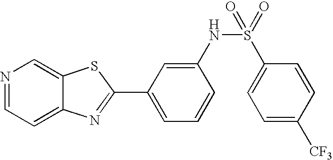 | | NA | C |
| 238 | 480.5 | 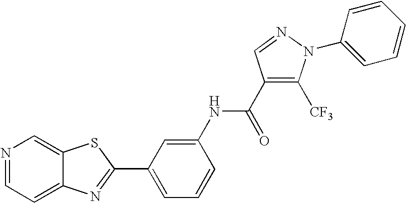 | | A′ | B |
| 239 | 466.5 | 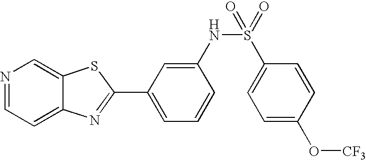 | | A | B |
| 240 | 392.4 | 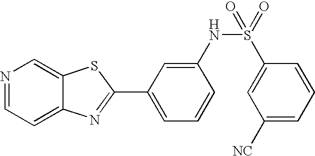 | | NA | C |
| 241 | 397.2 | 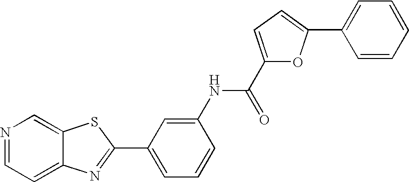 | | A′ | B |
| 244 | 332.4 | 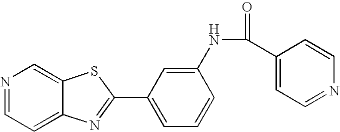 | | NA | C |
| 245 | 423.5 | 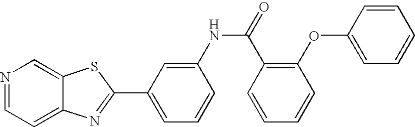 | | A′ | B |
| 246 | 391.5 | 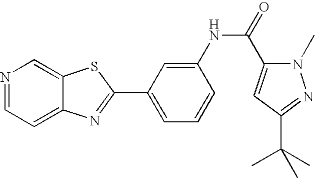 | | A′ | B |
| 247 | 413.5 | 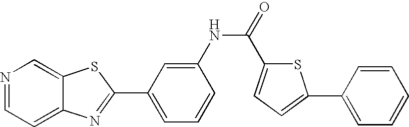 | | NA | C |
| 248 | 467.4 | 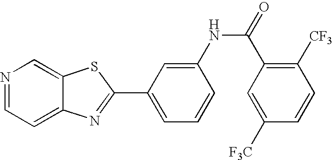 | | A | B |
| 249 | 395.9 | 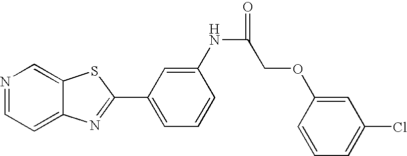 | | NA | C |
| 250 | 385.5 | 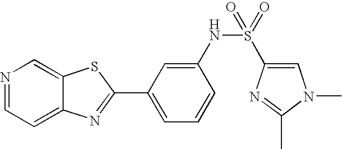 | | NA | C |
| 251 | 425.5 | 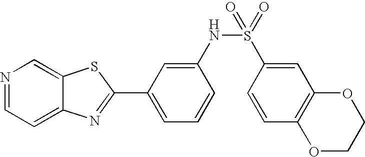 | | NA | C |
| 252 | 453.5 | 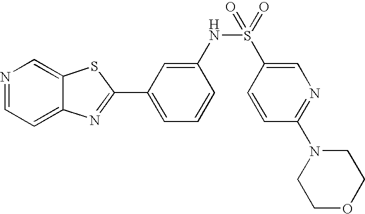 | | NA | C |
| 253 | 373.1 | 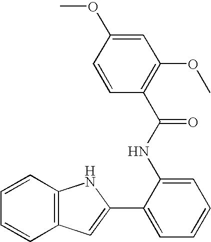 | | NA | C |
| 254 | 373 | 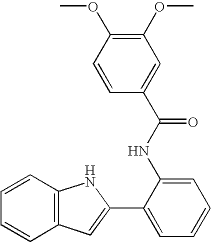 | | A′ | B |
| 255 | 403 | 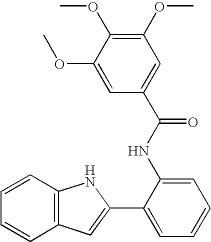 | | A′ | B |
| 256 | 356 | 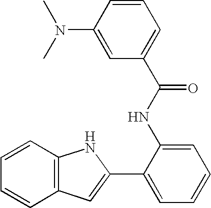 | | NA | C |
| 257 | 413.1 | 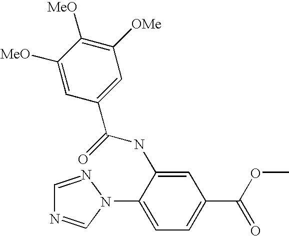 | | NA | C |
| 258 | 383.1 | 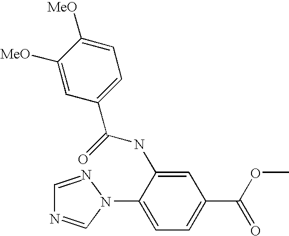 | | A | B |
| 259 | 405.1 | 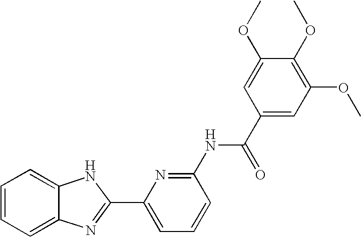 | | A′ | A |
| 260 | 375.1 | 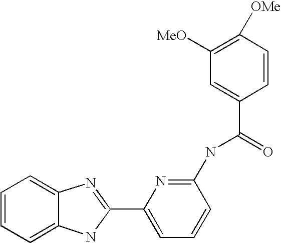 | | A′ | A |
| 261 | 375.1 | 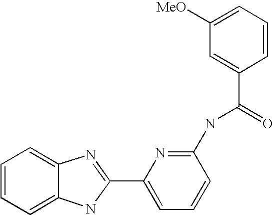 | | A′ | A |
| 262 | 374.1 | 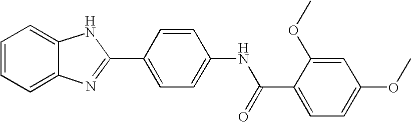 | | A′ | B |
| 263 | 404 | 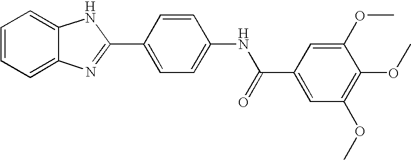 | | A | B |
| 264 | 357 | 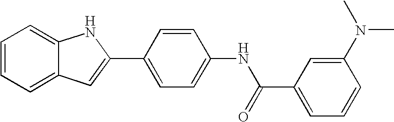 | | A′ | B |
| 265 | 374.1 | 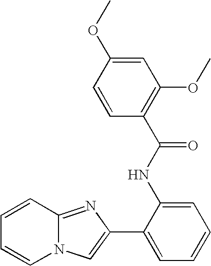 | | A | B |
| 266 | 374 | 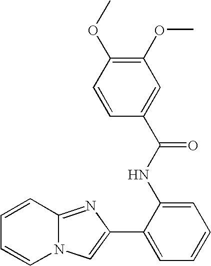 | | A′ | A |
| 267 | 404 | 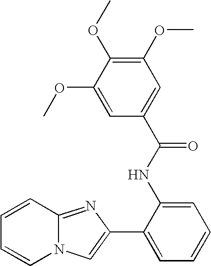 | | A′ | B |
| 268 | 357 | 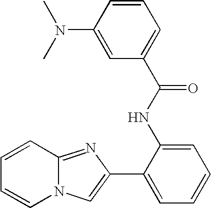 | | A′ | B |
| 270 | 478 | 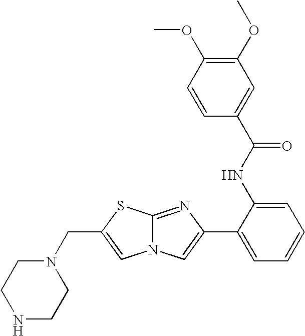 | | A | A |
| 271 | 508 | 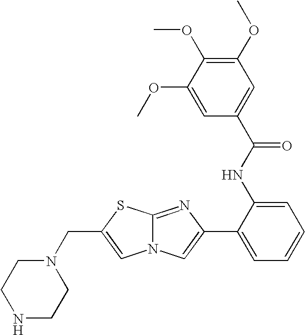 | | A′ | A |
| 272 | 392 | 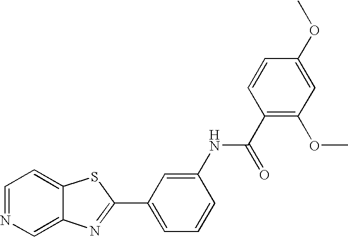 | | A′ | B |
| 273 | 422 | 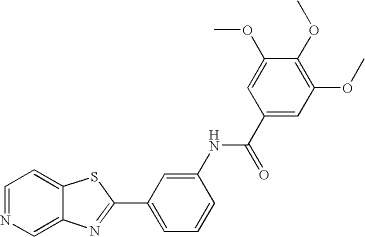 | | A | A |
| 276 | 375.1 | 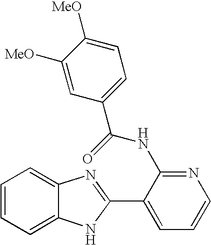 | | A′ | B |
| 280 | 381.5 | 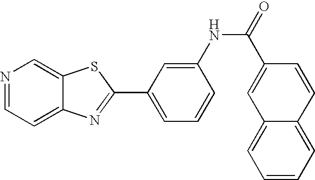 | | | |
| 282 | 386.5 | 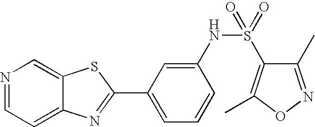 | | NA | |
| 283 | 451.6 | 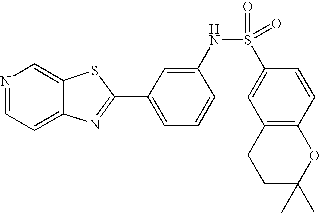 | | NA | C |
| 284 | 439.5 | 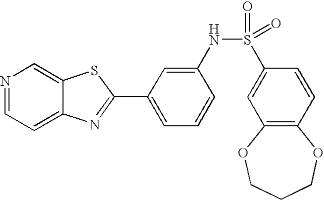 | | NA | C |
| 285 | 440.5 | 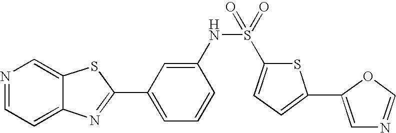 | | NA | C |
| 286 | 390.1 | 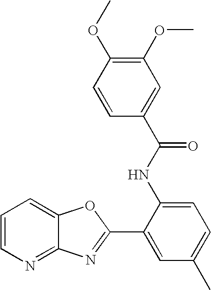 | | NA | C |
| 287 | 457.6 | 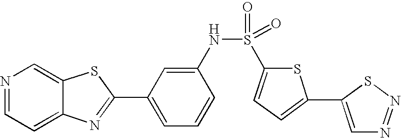 | | | |
| 288 | 424.5 | 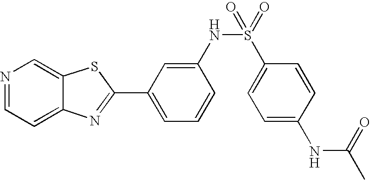 | | A | B |
| 289 | 427.5 | 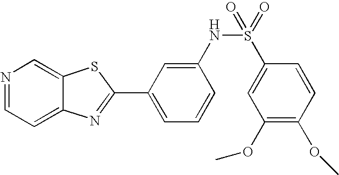 | | A | B |
| 290 | 445.5 | 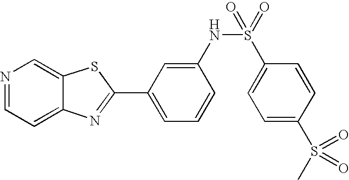 | | NA | C |
| 292 | 420.1 | 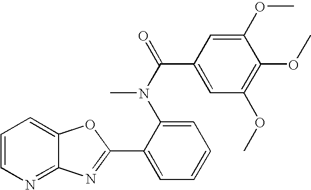 | | D | |
| 293 | 404.1 | 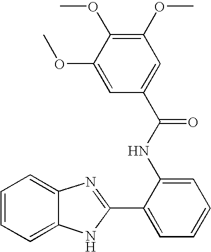 | | A′ | A |
| 294 | 374 | 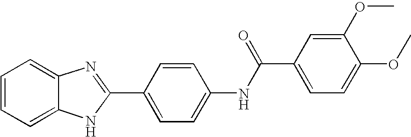 | | A′ | B |
| 295 | 252.1 | 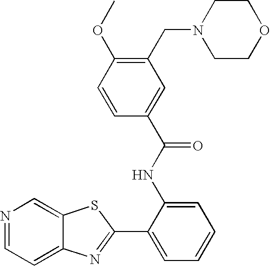 | | A′ | B |
| 296 | 376 | 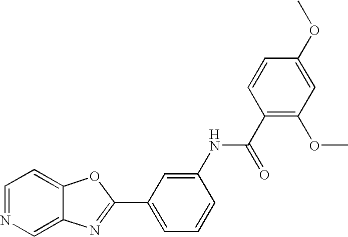 | | A | B |
| 297 | 376 | 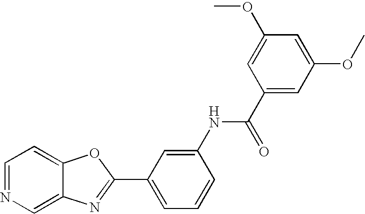 | | A | B |
| 298 | 406 | 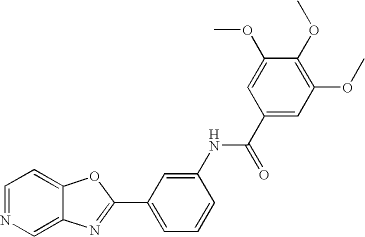 | | A | B |
| 299 | 359 | 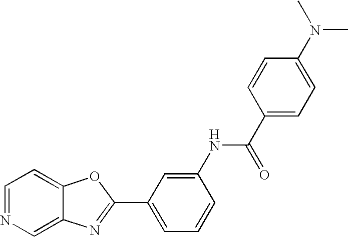 | | A | B |
| 303 | 437.5 | 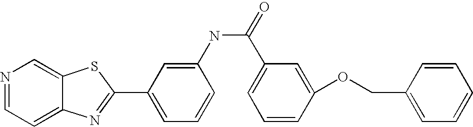 | | NA | |
| 304 | 336.4 | 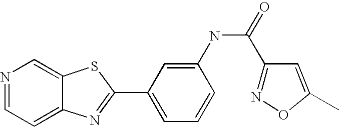 | | A′ | B |
| 305 | 414.5 | 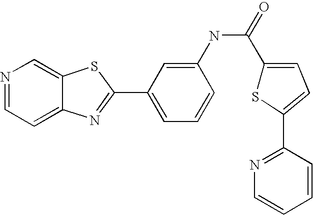 | | NA | |
| 306 | 424.5 | 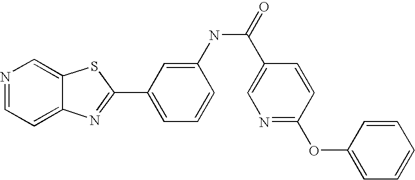 | | A | B |
| 307 | 382.4 | 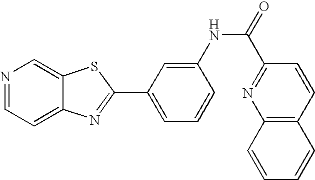 | | A′ | A |
| 308 | 400.3 | 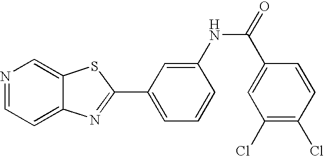 | | NA | |
| 309 | 367.4 | 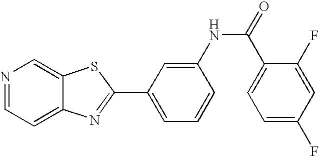 | | NA | |
| 310 | 387.5 | 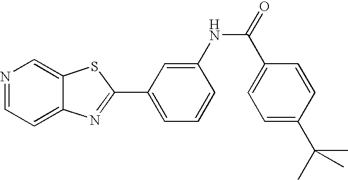 | | NA | |
| 311 | 359 | 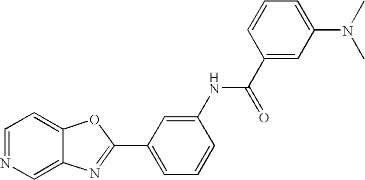 | | A | B |
| 313 | 418.1 | 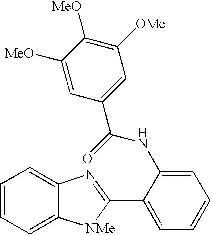 | | A | B |
| 314 | 388.1 | 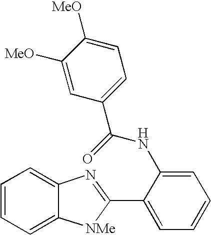 | | A | B |
| 315 | 388.1 | 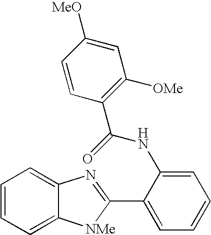 | | NA | |
| 317 | 392 | 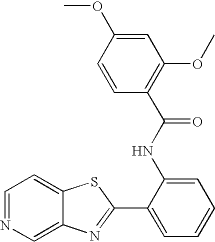 | | A′ | B |
| 318 | 392 | 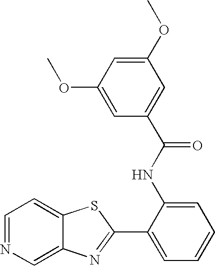 | | A | B |
| 319 | 422 | 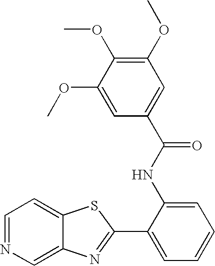 | | A′ | B |
| 320 | 375 | 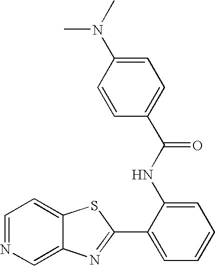 | | A′ | B |
| 321 | 375 | 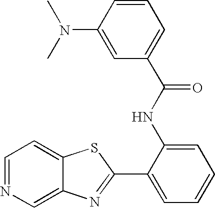 | | A′ | B |
| 322 | 392 | 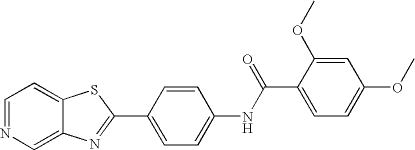 | | NA | |
| 323 | 392 | 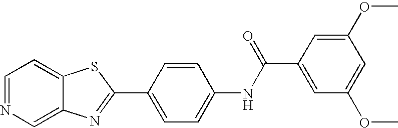 | | A | B |
| 324 | 422 | 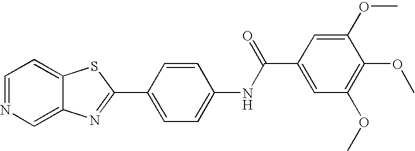 | | NA | |
| 325 | 375 | 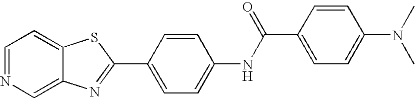 | | NA | |
| 326 | 478 | 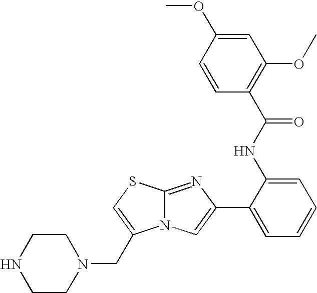 | | A | B |
| 327 | 461 | 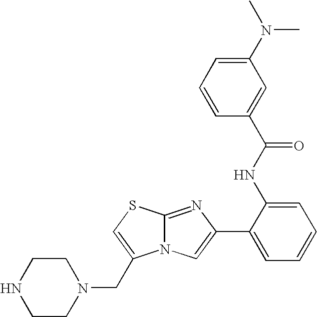 | | A | A |
| 328 | 418 | 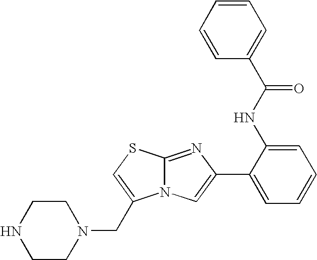 | | B | A |
| 329 | 462 | 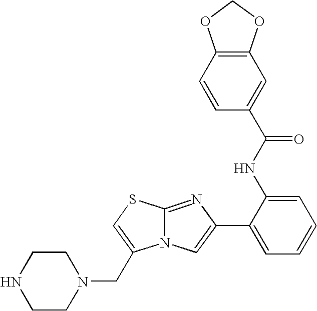 | | A | A |
| 330 | 443 | 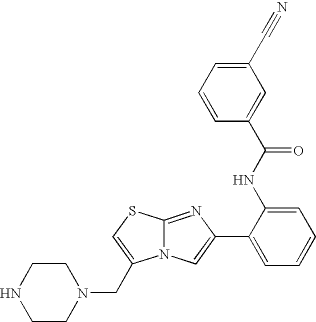 | | A | A |
| 331 | 335 | 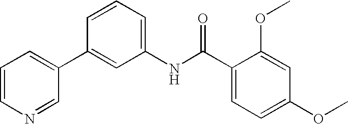 | | A | B |
| 332 | 335.1 | 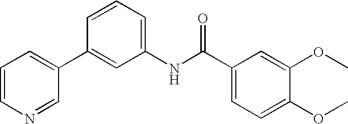 | | A | B |
| 333 | 365 | 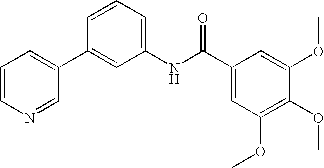 | | A | B |
| 334 | 340.1 | 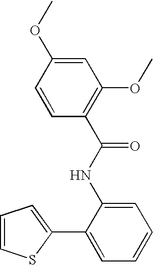 | | A | B |
| 335 | 340 | 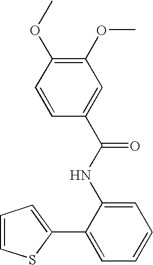 | | A | B |
| 336 10 | 370 | 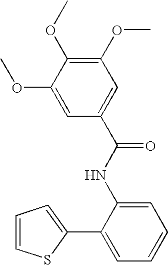 | | A | B |
| 337 | 443 | 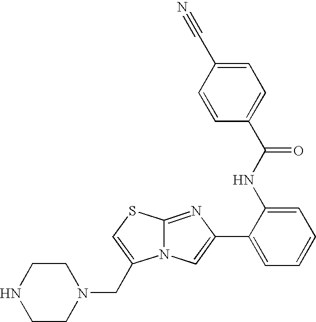 | | NA | |
| 338 | 458 | 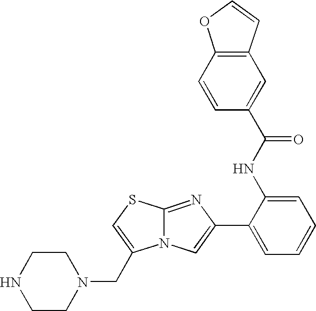 | | A | A |
| 339 | 376 | 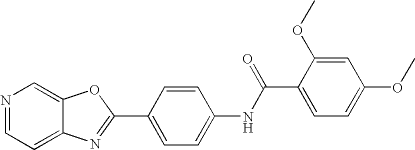 | | NA | |
| 340 | 376 | 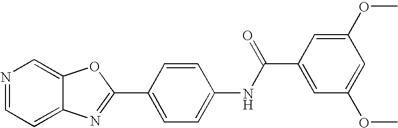 | | NA | |
| 341 | 406 | 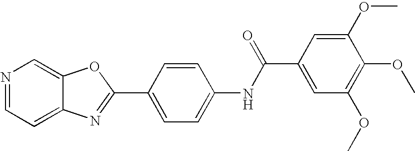 | | A′ | B |
| 342 | 359 | 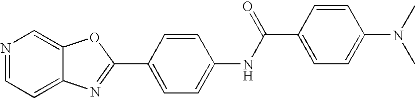 | | NA | |
| 343 | 406 | 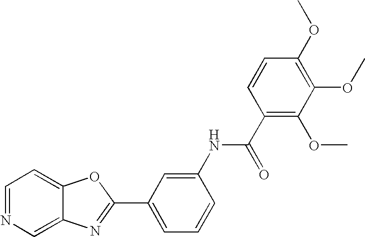 | | A | B |
| 344 | 375 | 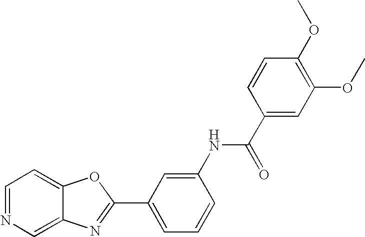 | | A′ | B |
| 345 | 376 | 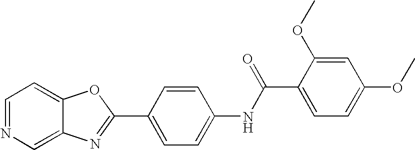 | | NA | |
| 346 | 376 | 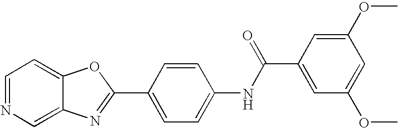 | | NA | |
| 347 | 406 | 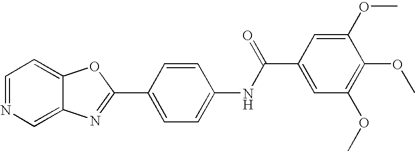 | | A′ | B |
| 348 | 359 | 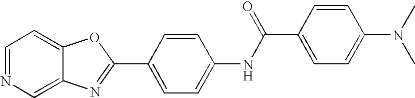 | | NA | |
| 349 | 375 | 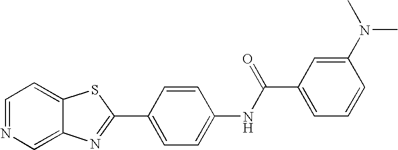 | | NA | |
| 350 | 431.1 | 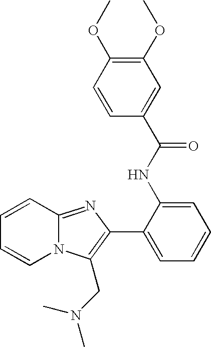 | | B | B |
| 351 | 461 | 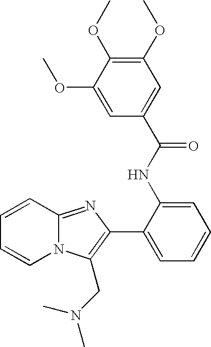 | | A | B |
| 359 | 472.1 | 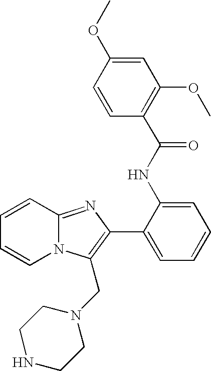 | | NA | |
| 362 | 502 | 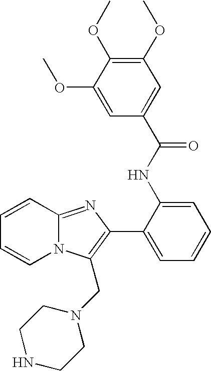 | | B | B |
| 364 | 472 | 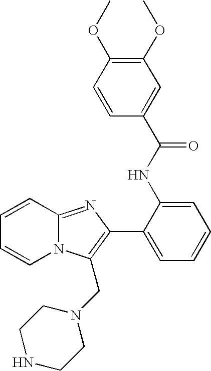 | | D | A |
| 367 | 359.1 | 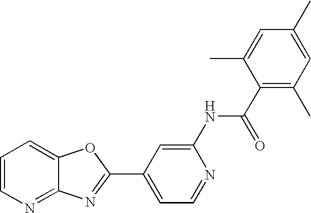 | | NA | |
| 369 | 350.8 | 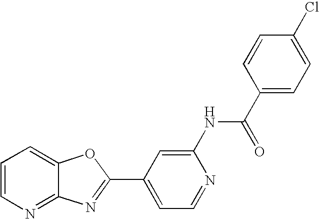 | | NA | |
| 370 | 437.0 | 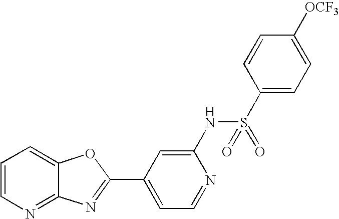 | | NA | |
| 371 | 381.1 | 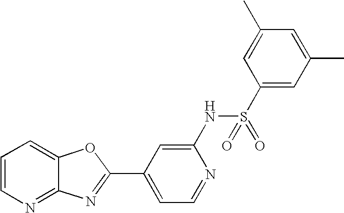 | | NA | |
| 372 | 431.1 | 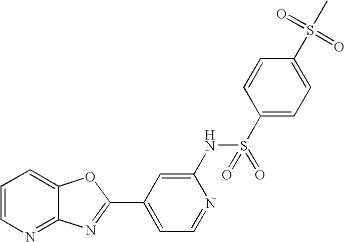 | | NA | |
| 373 | 445.0 | 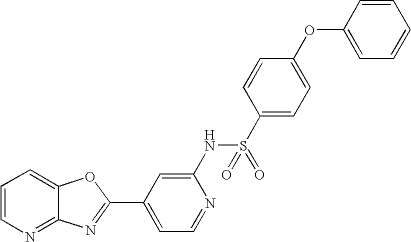 | | NA | |
| 374 | 421.1 | 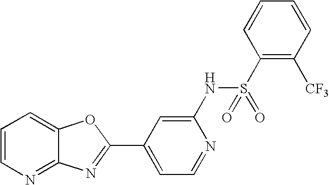 | | NA | |
| 375 | 358.2 | 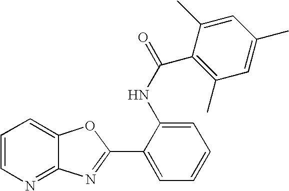 | | NA | |
| 376 | 350.0 | 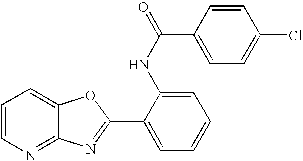 | | NA | |
| 377 | | 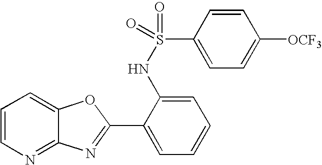 | | NA | |
| 378 | 412.1 | 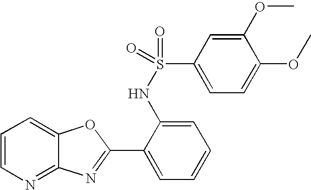 | | NA | |
| 379 | 380.0 | 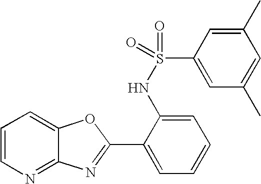 | | NA | |
| 380 | 429.9 | 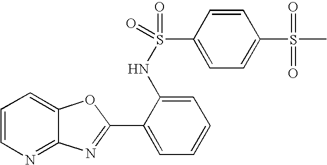 | | NA | |
| 381 | 444.1 | 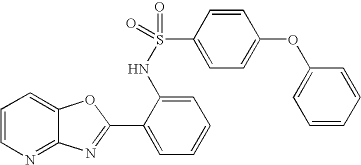 | | NA | |
| 382 | 420.0 | 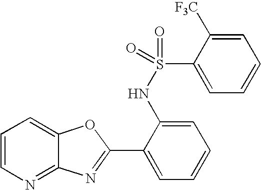 | | NA | |
| 383 | 515.7 | 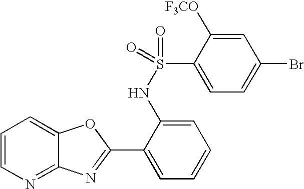 | | NA | |
| 384 | 487.8 | 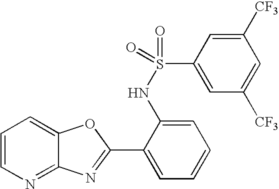 | | NA | |
| 385 | 397.2 | 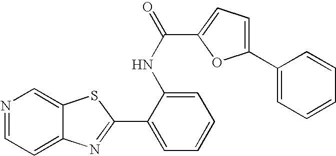 | | NA | |
| 387 | 366.8 | 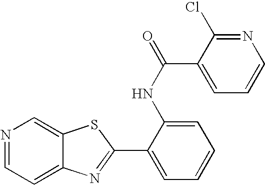 | | NA | |
| 390 | 412.5 | 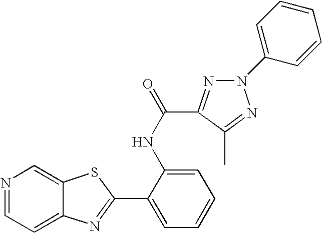 | | A | B |
| 391 | 333.4 | 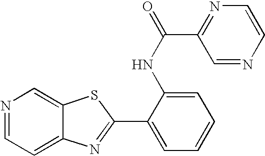 | | A | B |
| 392 | 424.5 | 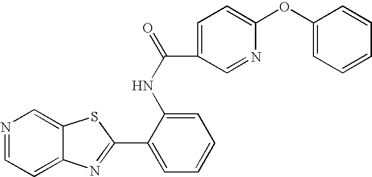 | | A | B |
| 393 | 400.3 | 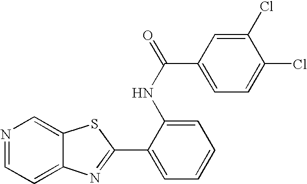 | | NA | |
| 394 | 457.6 | 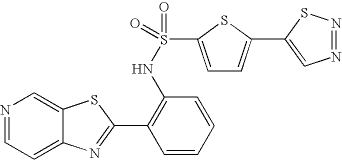 | | NA | |
| 396 | 389.5 | 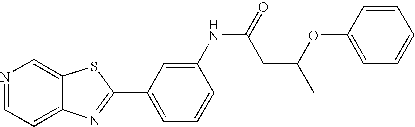 | | A | B |
| 398 | 460.1 | 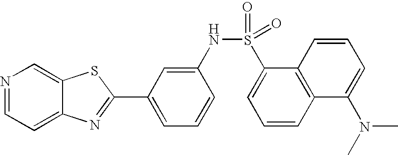 | | A | B |
| 399 | 424.5 | 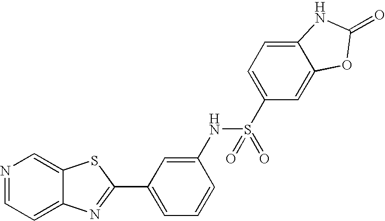 | | A | B |
| 400 | 392 | 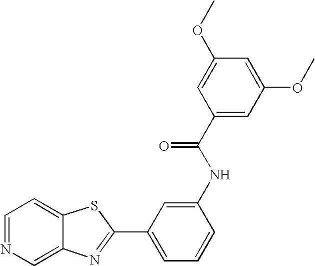 | | NA | |
| 401 | 422 | 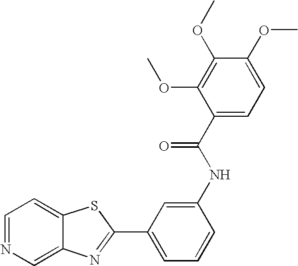 | | A | B |
| 402 | 375 | 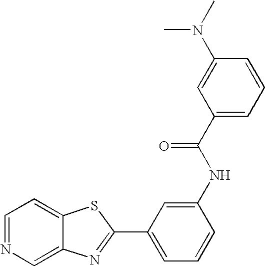 | | A′ | B |
| 403 | 375 | 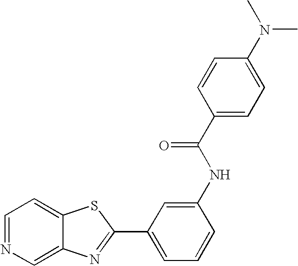 | | A′ | B |
| 404 | 376 | 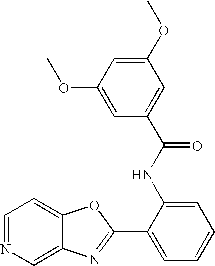 | | A′ | B |
| 405 | 406 | 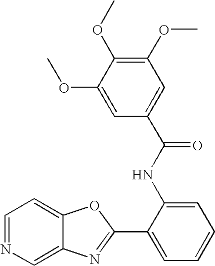 | | A′ | B |
| 406 | 359 | 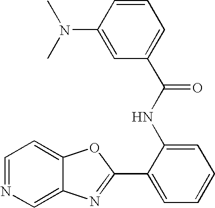 | | A′ | B |
| 407 | 359 | 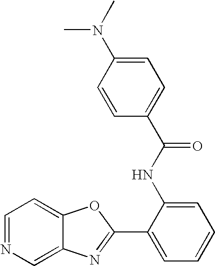 | | A′ | B |
| 408 | 359 | 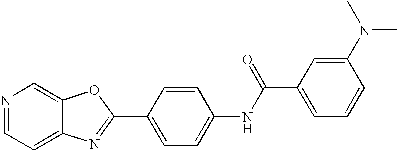 | | B | B |
| 409 | 422 | 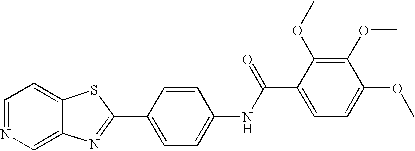 | | NA | |
| 410 | 359 | 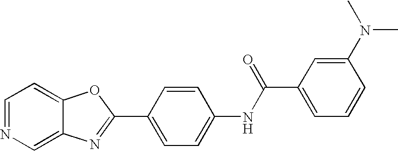 | | NA | |
| 411 | 422 | 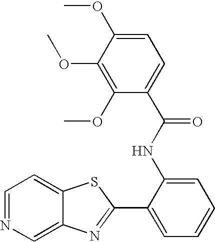 | | A | B |
| 412 | 406 | 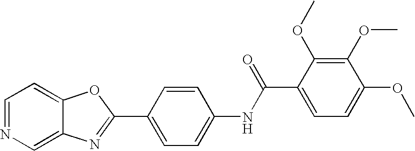 | | NA | |
| 413 | 385.1 | 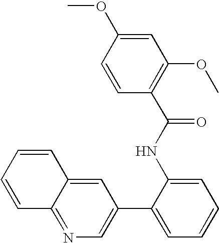 | | B | B |
| 414 | 385 | 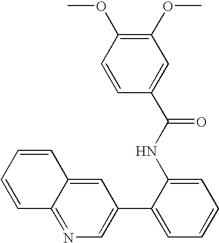 | | A | B |
| 415 | 415 | 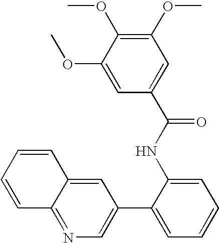 | | B | B |
| 416 | 368 | 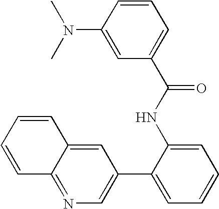 | | NA | |
| 419 | 406 | 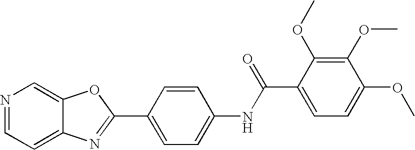 | | NA | |
| 420 | 376 | 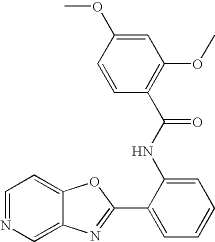 | | NA | |
| 421 | 406 | 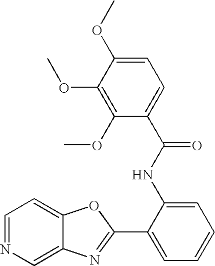 | | A | B |
| 422 | 382 | 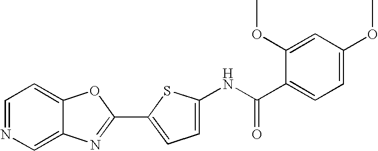 | | A | B |
| 423 | 382 | 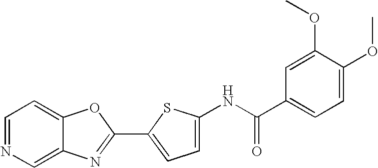 | | A′ | B |
| 424 | 382 | 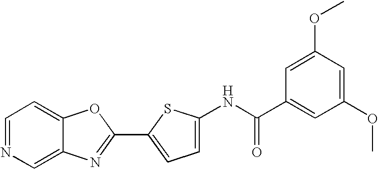 | | NA | |
| 425 | 412 | 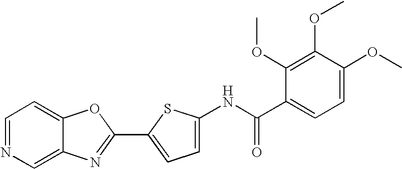 | | NA | |
| 426 | 412 | 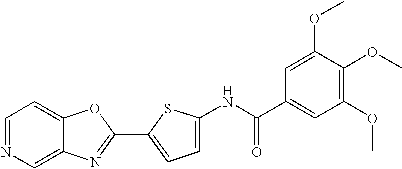 | | A′ | B |
| 427 | 365 | 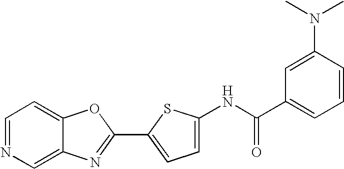 | | A′ | B |
| 428 | 365 | 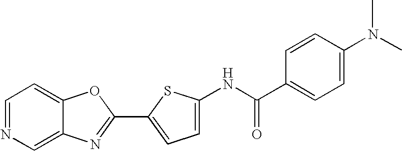 | | NA | |
| 429 | 376 | 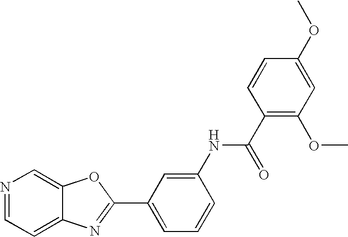 | | A′ | B |
| 430 | 406 | 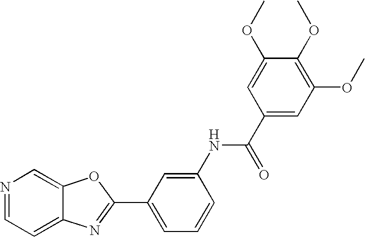 | | A | A |
| 431 | 359 | 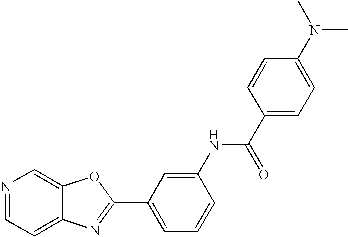 | | A | B |
| 436 | 445.1 | 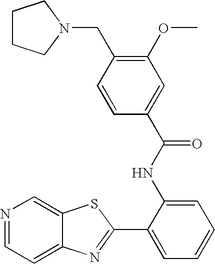 | | A | A |
| 437 | 375.1 | 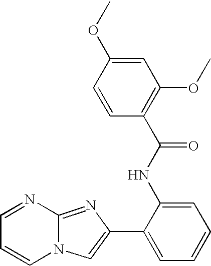 | | A | B |
| 438 | 375 | 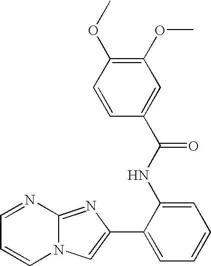 | | A′ | A |
| 439 | 405 | 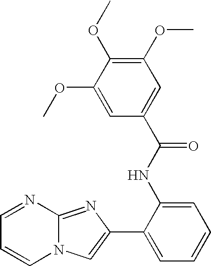 | | A′ | B |
| 440 | 468 | 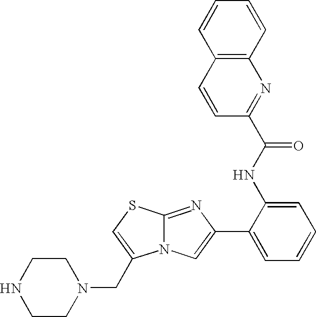 | | A′ | A |
| 441 | 470 | 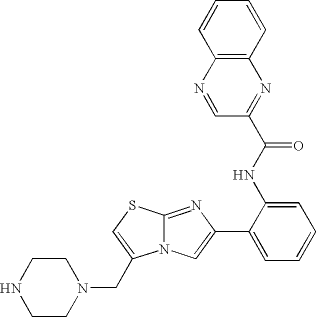 | | A′ | A |
| 442 | 472 | 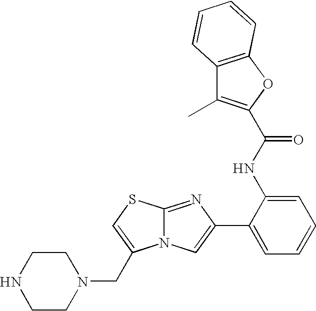 | | A′ | A |
| 443 | 436 | 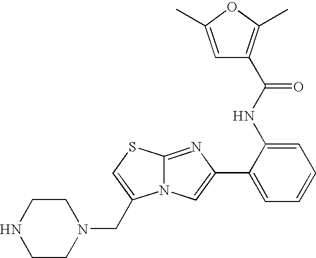 | | A | A |
| 444 | 464 | 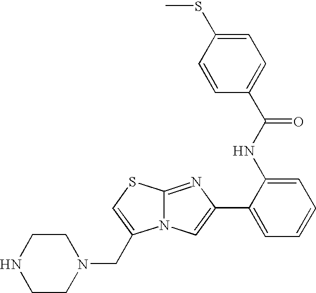 | | A | A |
| 445 | 432 | 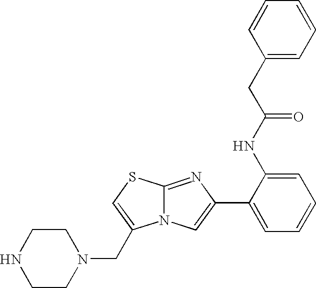 | | A | B |
| 446 | 424 | 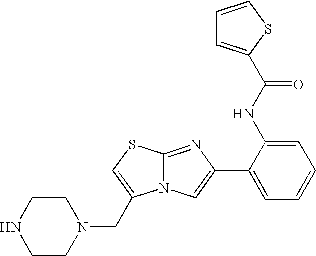 | | A | B |
| 447 | 484 | 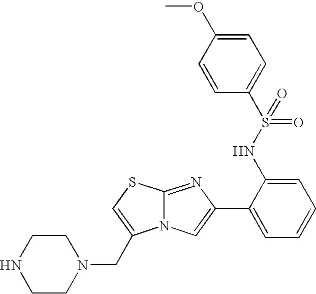 | | NA | |
| 448 | 510 | 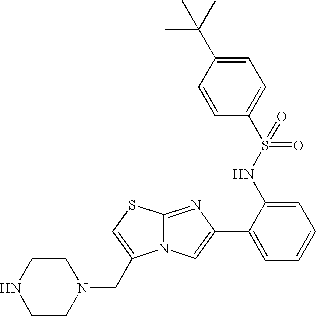 | | NA | |
| 449 | 376 | 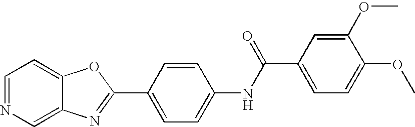 | | NA | |
| 450 | 392 | 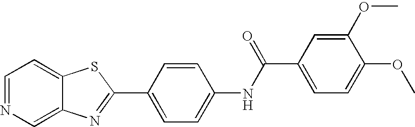 | | NA | |
| 451 | 376 | 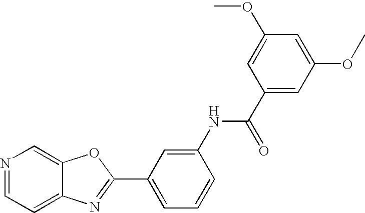 | | A | B |
| 452 | 406 | 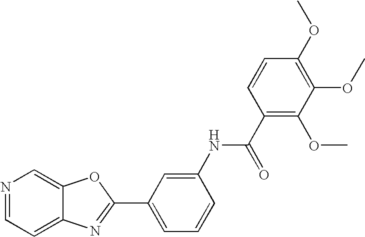 | | A | B |
| 453 | 359 | 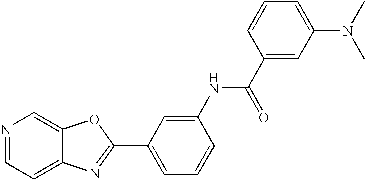 | | A | B |
| 454 | 376 | 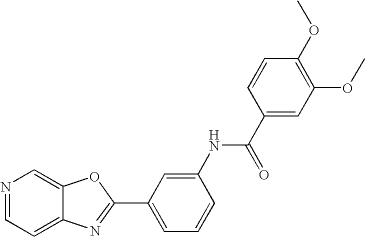 | | A | B |
| 455 | 376 | 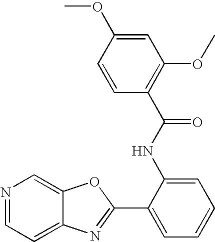 | | A′ | B |
| 456 | 376 | 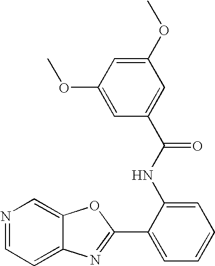 | | A′ | B |
| 457 | 406 | 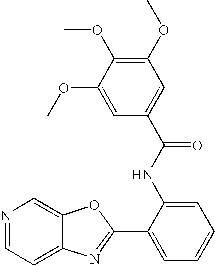 | | A | B |
| 458 | 359 | 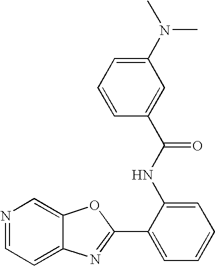 | | A′ | B |
| 459 | 359 | 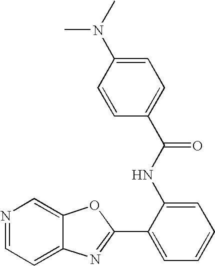 | | A′ | B |
| 460 | 359.4 | 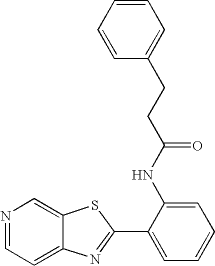 | | A | B |
| 461 | 367.4 | 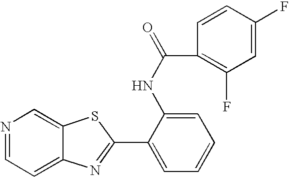 | | NA | |
| 462 | 391.5 | 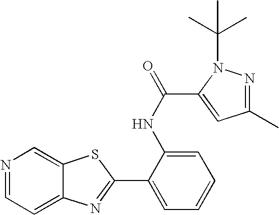 | | A′ | B |
| 463 | 375.4 | 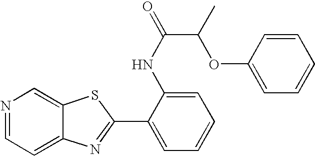 | | A | B |
| 465 | 395.9 | 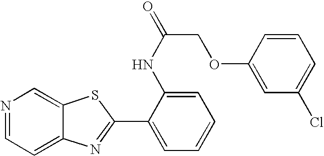 | | NA | |
| 466 | 445.5 | 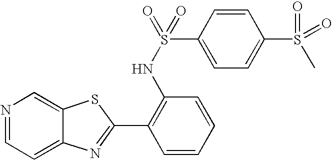 | | NA | |
| 467 | 427.2 | 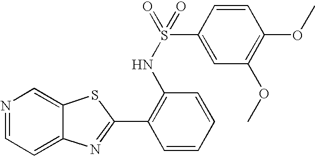 | | NA | |
| 468 | 345.0 | 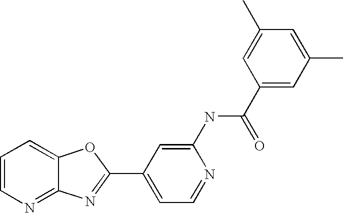 | | A | B |
| 469 | 435.4 | 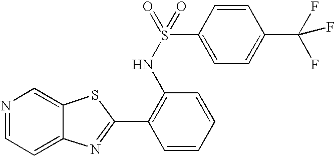 | | NA | |
| 470 | 365.0 | 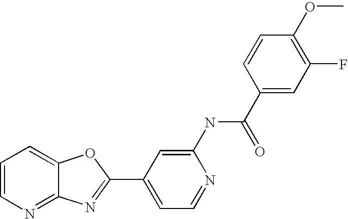 | | NA | |
| 471 | 488.9 | 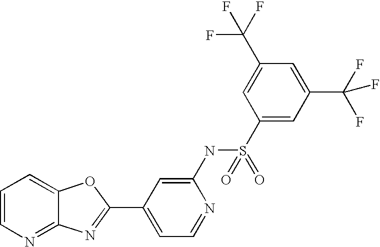 | | NA | |
| 472 | 437.5 | 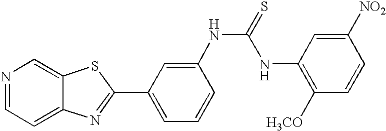 | | NA | |
| 473 | 420.5 | 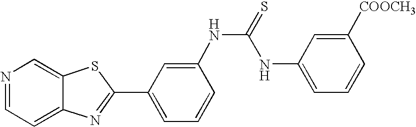 | | A′ | B |
| 474 | | 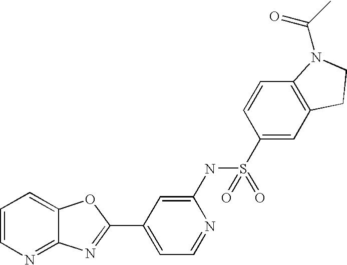 | | NA | |
| 475 | 408.6 | 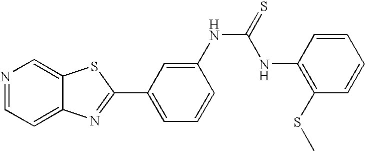 | | NA | |
| 476 | 410.9 | 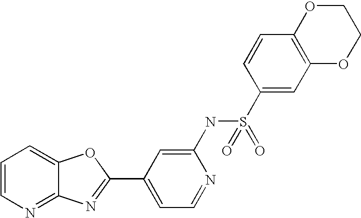 | | NA | |
| 477 | 435.9 | 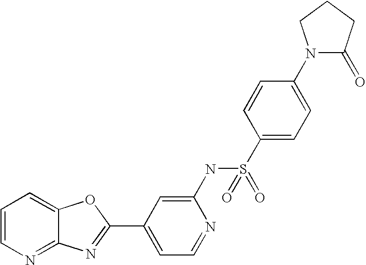 | | NA | |
| 478 | 404.8 | 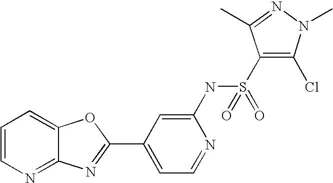 | | NA | |
| 479 | 420.9 | 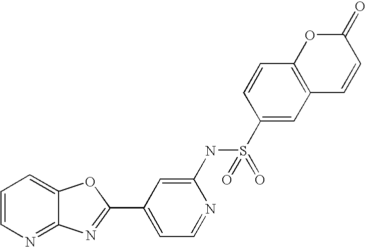 | | NA | |
| 481 | 428.5 | 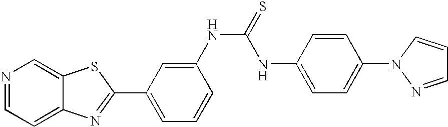 | | A | B |
| 482 | 344.1 | 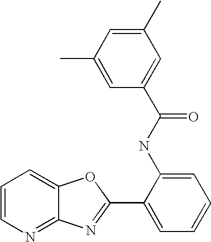 | | A′ | B |
| 483 | 364.1 | 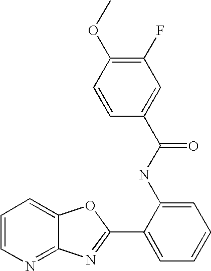 | | NA | |
| 484 | 448.6 | 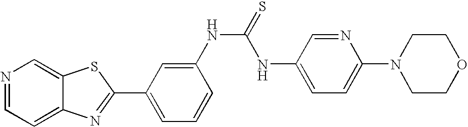 | | A | B |
| 485 | 363.5 | 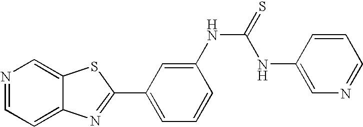 | | A | A |
| 486 | 385.9 | 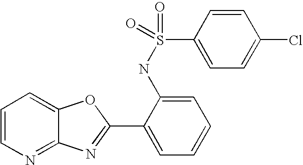 | | NA | |
| 487 | 396.1 | 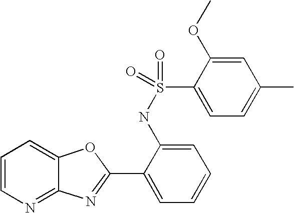 | | NA | |
| 488 | 435.1 | 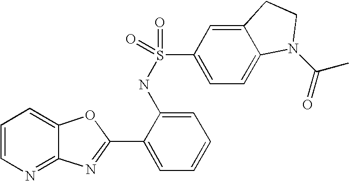 | | NA | |
| 489 | 410.1 | 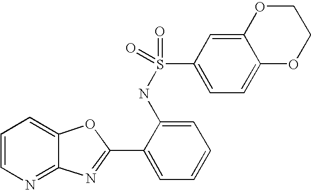 | | NA | |
| 490 | 434.9 | 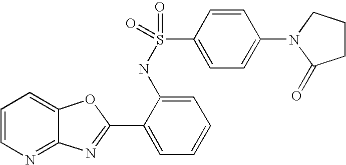 | | NA | |
| 491 | 420.5 | 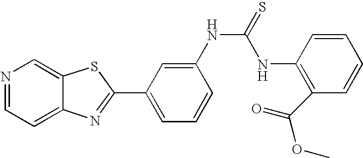 | | NA | |
| 492 | 404.0 | 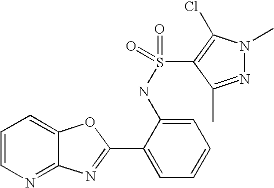 | | NA | |
| 493 | | 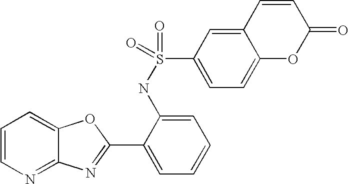 | | NA | |
| 494 | 422.9 | 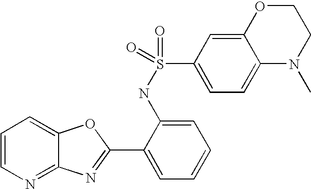 | | NA | |
| 495 | 366.0 | 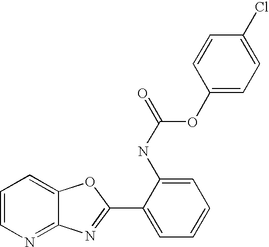 | | NA | |
| 496 | 349.9 | 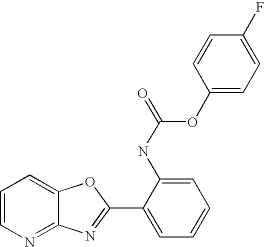 | | NA | |
| 497 | 346.0 | 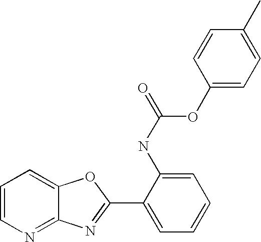 | | NA | |
| 498 | 406.5 | 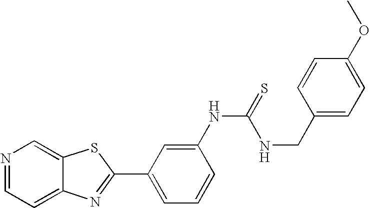 | | A | B |
| 499 | 362.1 | 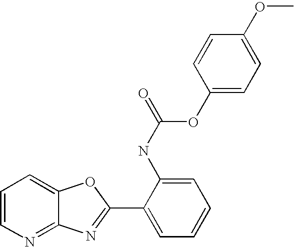 | | NA | |
| 500 | | 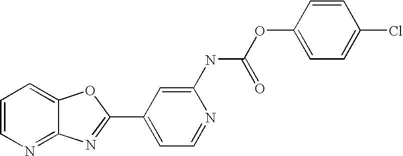 | | NA | |
| 501 | 347.1 | 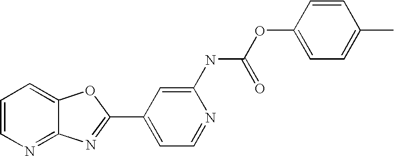 | | NA | |
| 502 | 363.1 | 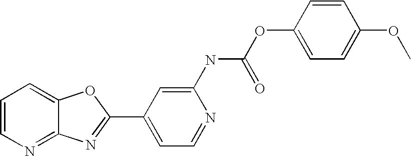 | | NA | |
| 503 | 443.1 | 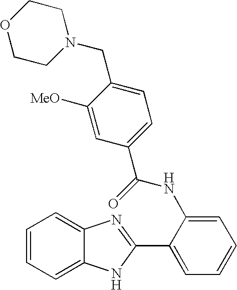 | | A′ | A |
| 504 | 358 | 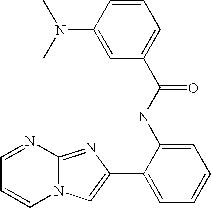 | | A | B |
| 505 | 359 | 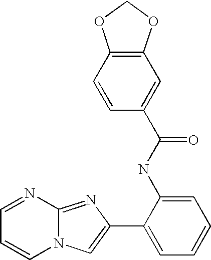 | | NA | |
| 506 | 429.1 | 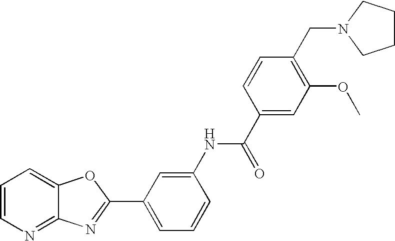 | | A | A |
| 507 | 388.1 | 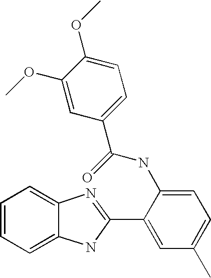 | | A′ | A |
| 508 | 366.1 | 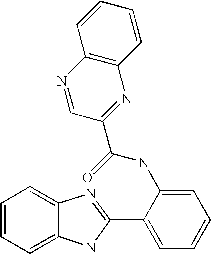 | | A′ | A |
| 510 | 457 | 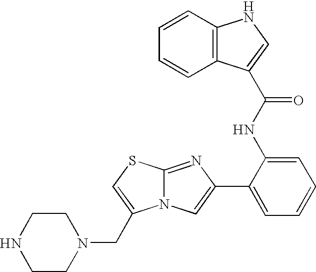 | | A′ | A |
| 511 | 460 | 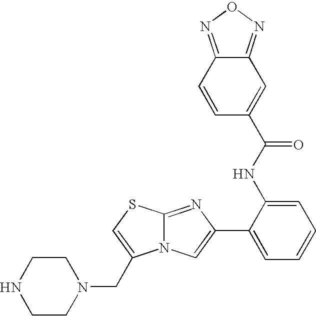 | | NA | |
| 512 | 484 | 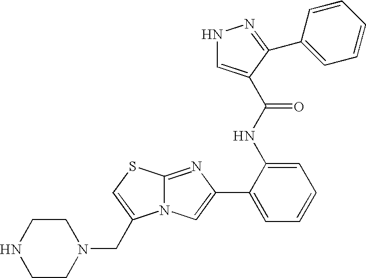 | | A′ | A |
| 513 | 470 | 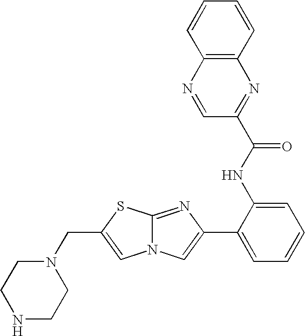 | | A′ | A |
| 514 | 466 | 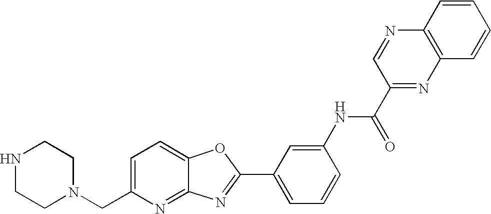 | | A | B |
| 515 | 398 | 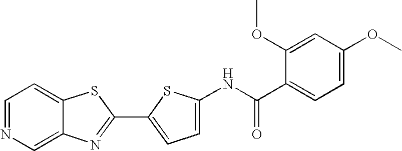 | | A′ | B |
| 516 | 398 | 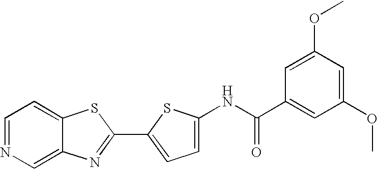 | | NA | |
| 517 | 428 | 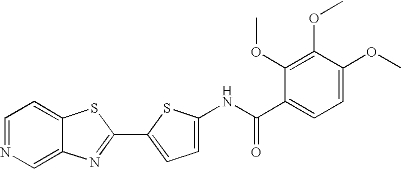 | | NA | |
| 518 | 381 | 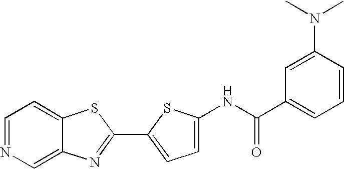 | | A′ | B |
| 519 | 381 | 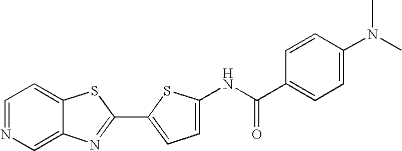 | | A′ | B |
| 520 | 428 | 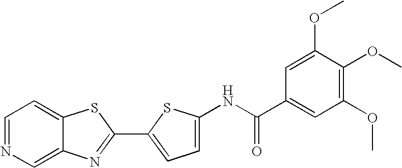 | | A′ | B |
| 521 | 375.0 | 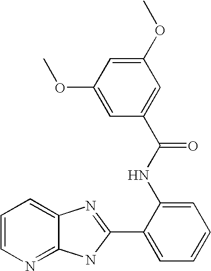 | | A′ | B |
| 522 | 405.0 | 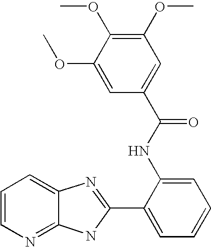 | | A | A |
| 523 | 359.0 | 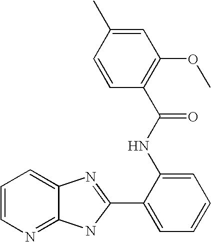 | | A | B |
| 524 | 358.0 | 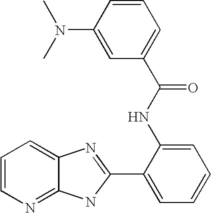 | | A′ | B |
| 525 | 375.0 | 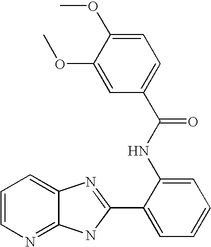 | | A′ | B |
| 526 | 400.0 | 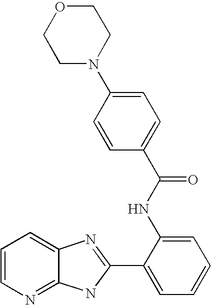 | | A′ | A |
| 527 | 398.0 | 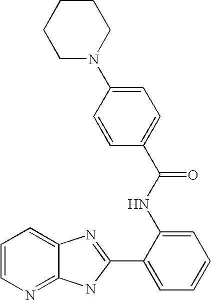 | | A′ | B |
| 528 | 412.9 | 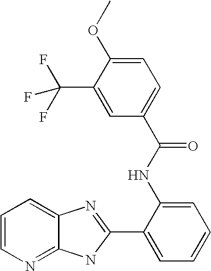 | | A′ | B |
| 529 | 399.1 | 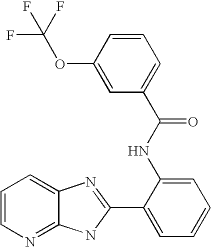 | | A′ | B |
| 530 | 359.0 | 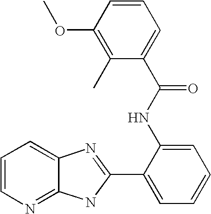 | | A′ | B |
| 531 | 345.0 | 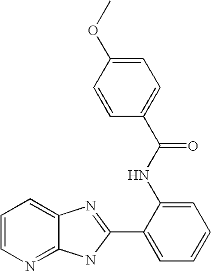 | | A′ | B |
| 532 | 345.0 | 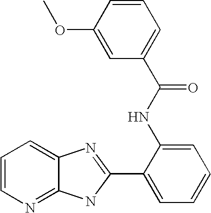 | | A′ | B |
| 533 | 315.0 | 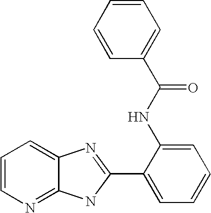 | | A′ | B |
| 534 | 358.0 | 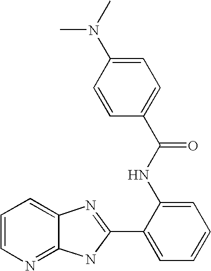 | | A′ | B |
| 535 | 428.1 | 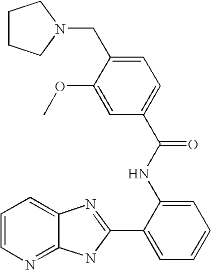 | | A | A |
| 536 | 442.1 | 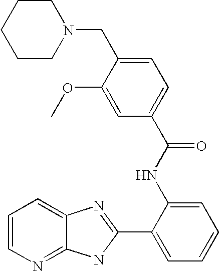 | | A | A |
| 537 | 444.0 | 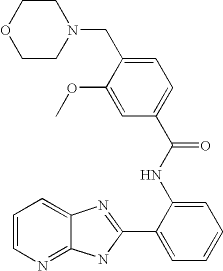 | | A′ | A |
| 538 | 443.1 | 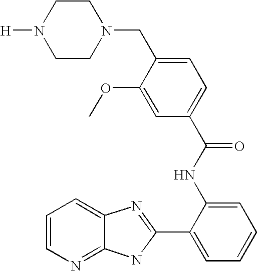 | | A | A |
| 539 | 457.1 | 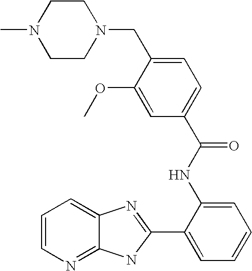 | | A′ | A |
| 540 | 402.1 | 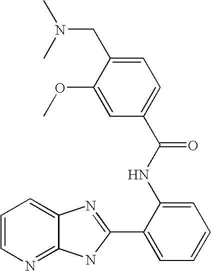 | | A | A |
| 541 | 443.1 | 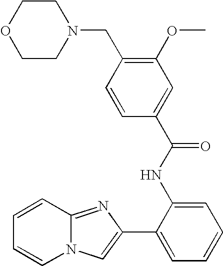 | | A | B |
| 542 | 444 | 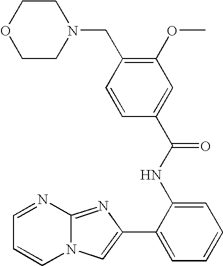 | | A | A |
| 543 | 420 | 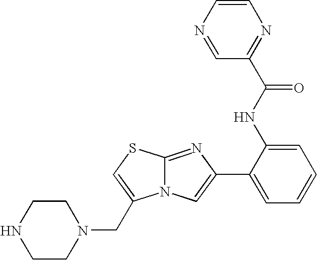 | | A′ | A |
| 544 | 474 | 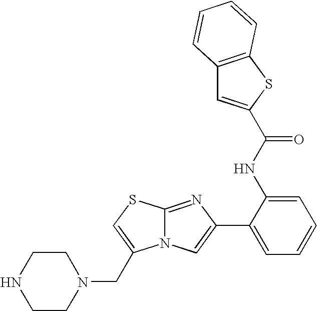 | | A | A |
| 545 | 378.1 | 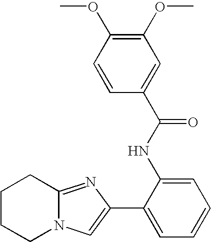 | | A | B |
| 546 | 408 | 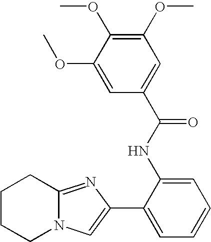 | | A | B |
| 547 | 369 | 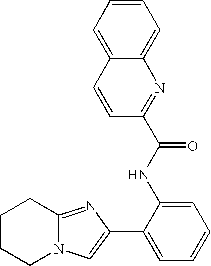 | | A′ | B |
| 548 | 370 | 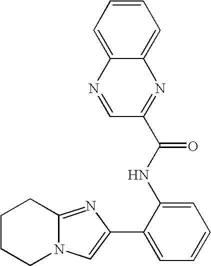 | | A′ | B |
| 556 | 365.1 | 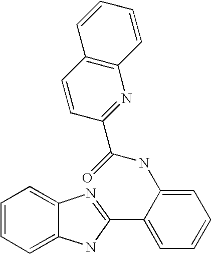 | | A′ | A |
| 557 | 542.1 | 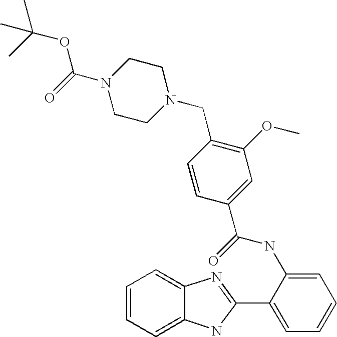 | | NA | |
| 558 | 442.1 | 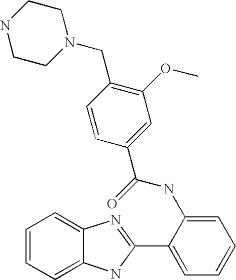 | | A′ | A |
| 559 | 508 | 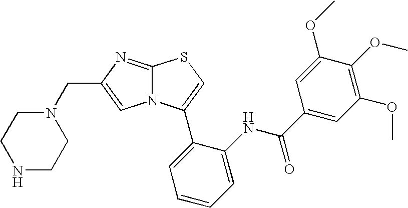 | | NA | |
| 560 | 470 | 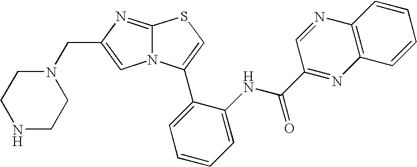 | | NA | |
| 561 | 382 | 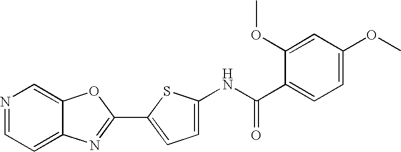 | | A′ | B |
| 562 | 382 | 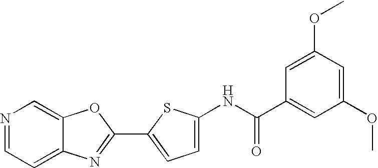 | | A′ | B |
| 563 | 412 | 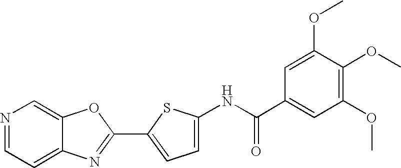 | | A | B |
| 565 | 400.8 | 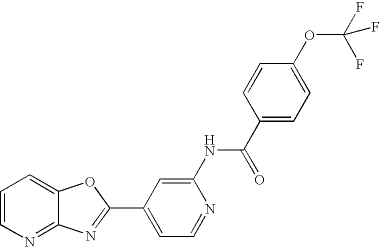 | | NA | |
| 566 | 409.9 | 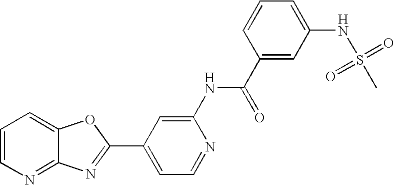 | | A | B |
| 567 | 374.9 | 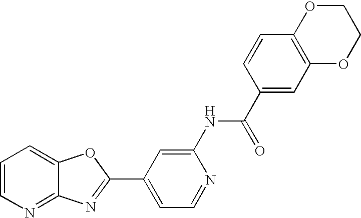 | | NA | |
| 568 | 367.0 | 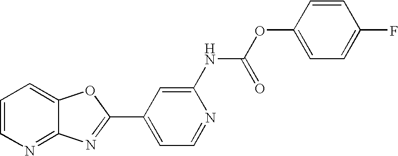 | | NA | |
| 569 | 374.9 | 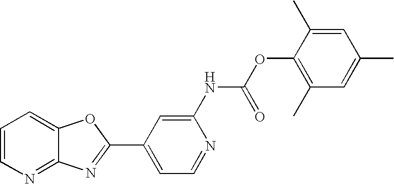 | | NA | |
| 570 | 361.0 | 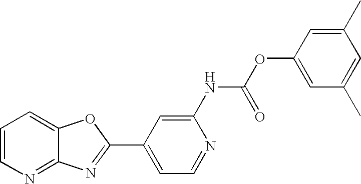 | | NA | |
| 571 | 444.0 | 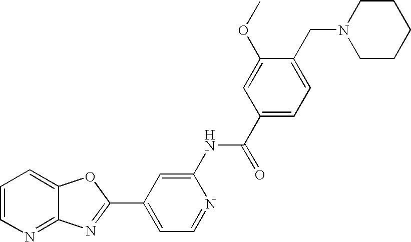 | | A | B |
| 572 | 429.9 | 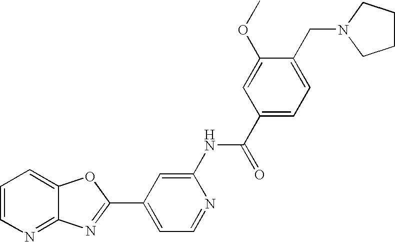 | | B | B |
| 573 | 431.8 | 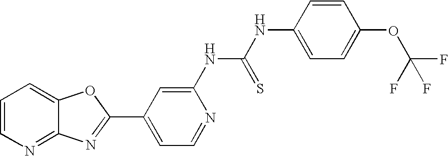 | | NA | |
| 574 | 376.2 | 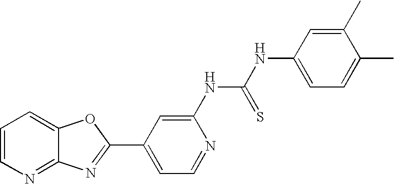 | | NA | |
| 575 | 439.9 | 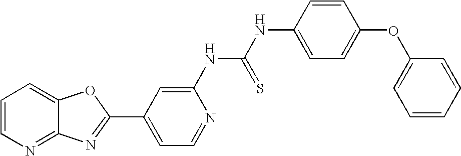 | | NA | |
| 576 | 376.1 | 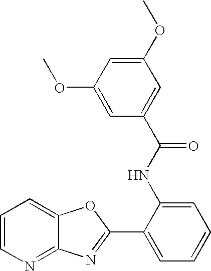 | | NA | |
| 577 | 400.1 | 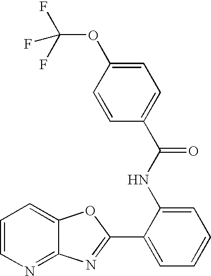 | | NA | |
| 578 | 368.1 | 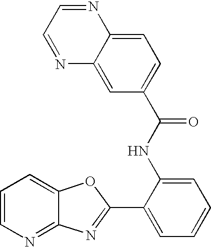 | | NA | |
| 579 | 401.1 | 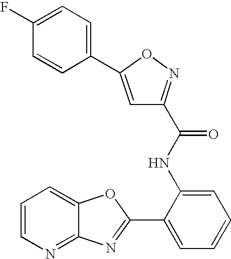 | | NA | |
| 580 | 374.0 | 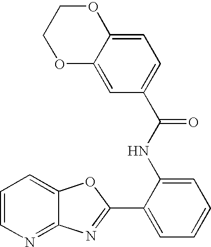 | | NA | |
| 581 | 334.0 | 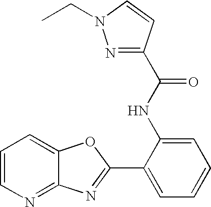 | | A | B |
| 582 | 400.0 | 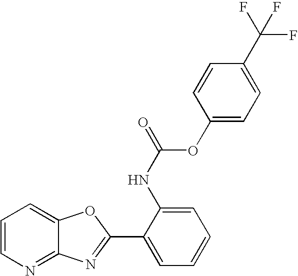 | | NA | |
| 583 | 374.1 | 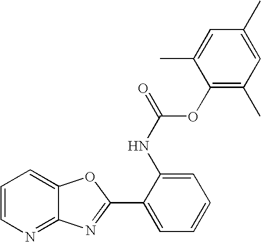 | | NA | |
| 584 | 360.0 | 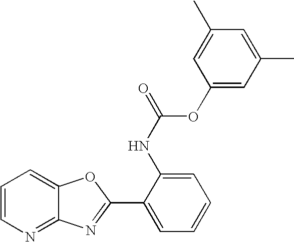 | | NA | |
| 585 | 443.1 | 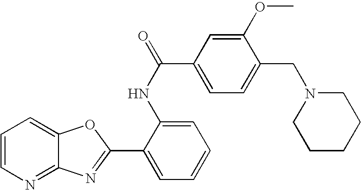 | | A | B |
| 587 | 427.1 | 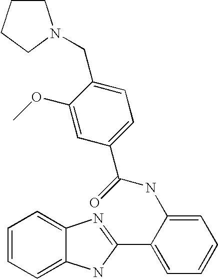 | | A′ | B |
| 588 | 520 | 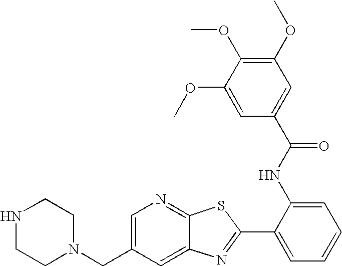 | | A | A |
| 589 | 490 | 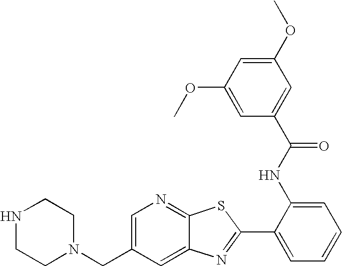 | | A′ | A |
| 590 | 474 | 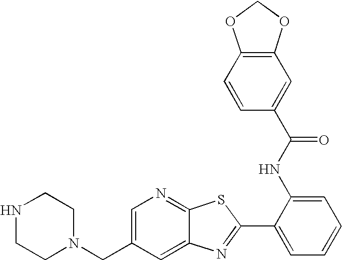 | | A | B |
| 591 | 458 | 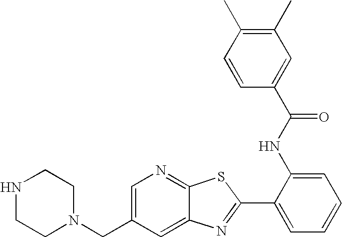 | | A′ | A |
| 592 | 455 | 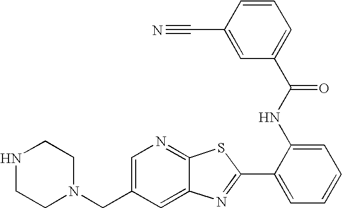 | | A′ | B |
| 593 | 473 | 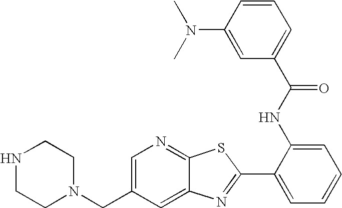 | | A′ | A |
| 594 | 466 | 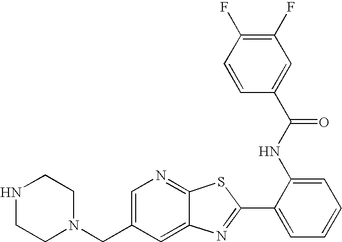 | | A | B |
| 596 | 467.4 | 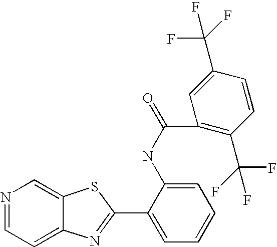 | | A′ | B |
| 597 | 377.2 | 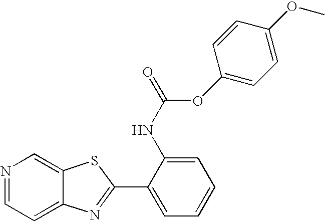 | | A′ | B |
| 599 | 422.3 | 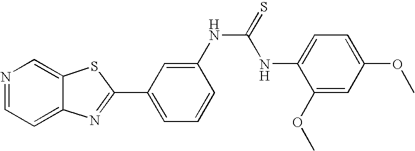 | | A′ | B |
| 600 | 422.5 | 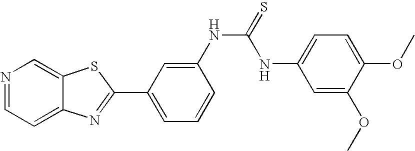 | | D | B |
| 601 | 405.5 | 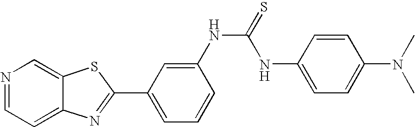 | | NA | |
| 604 | 360.4 | 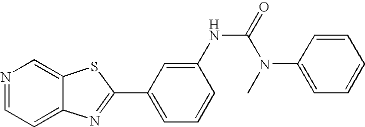 | | NA | |
| 605 | 377.4 | 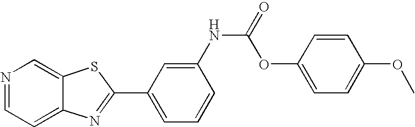 | | NA | |
| 607 | 415.4 | 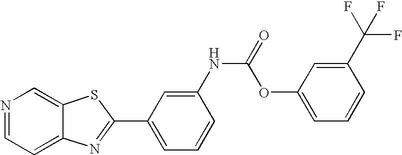 | | NA | |
| 608 | 332.4 | 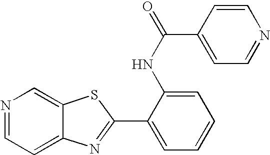 | | NA | |
| 609 | 439.3 | 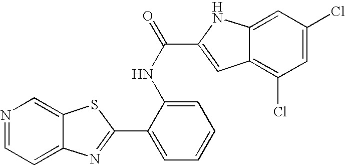 | | NA | |
| 610 | 418.6 | 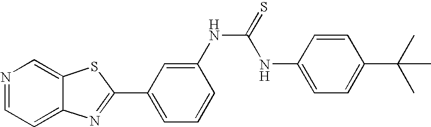 | | NA | |
| 611 | 465.6 | 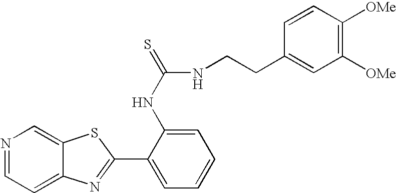 | | A′ | B |
| 612 | 402.5 | 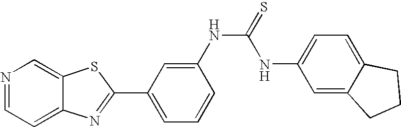 | | NA | |
| 614 | 428.5 | 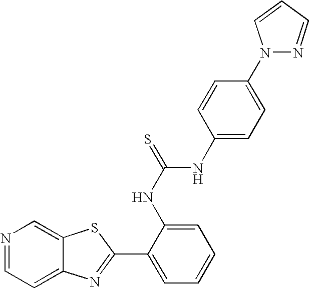 | | NA | |
| 615 | 408.6 | 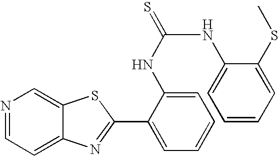 | | NA | |
| 616 | 437.5 | 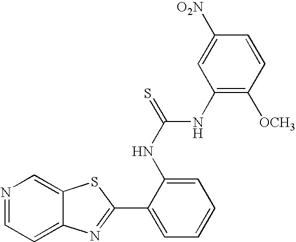 | | | |
| 617 | 471.1 | 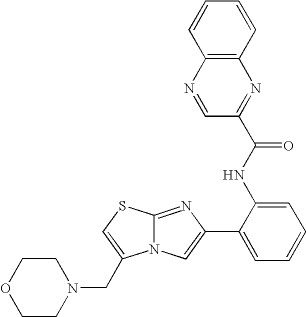 | | A′ | A |
| 618 | 469.1 | 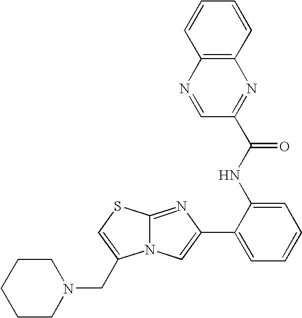 | | A′ | B |
| 619 | 455 | 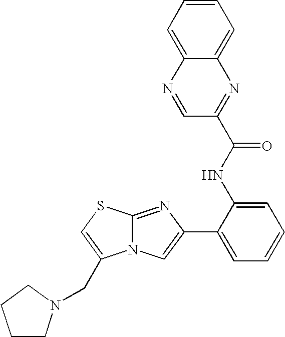 | | A′ | B |
| 620 | 493.1 | 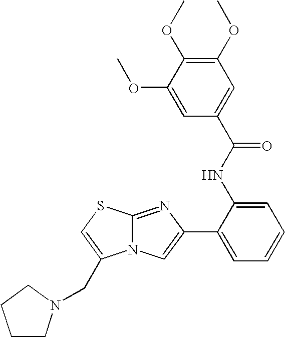 | | A′ | B |
| 621 | 472 | 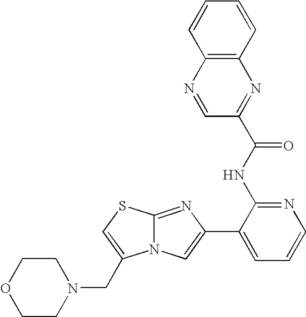 | | A′ | A |
| 622 | 482 | 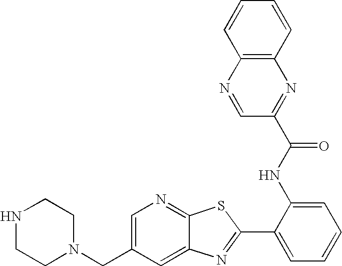 | | A′ | A |
| 623 | 437 | 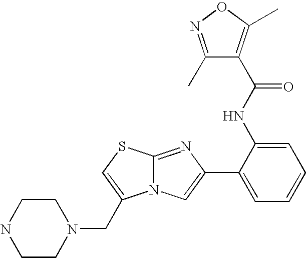 | | NA | C |
| 624 | 458 | 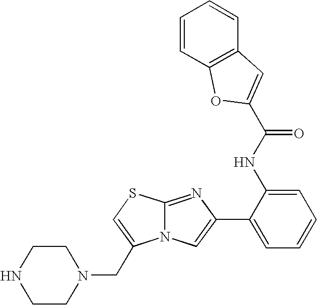 | | A′ | A |
| 625 | 496 | 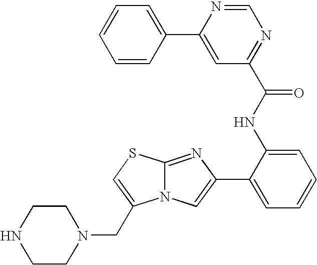 | | A′ | A |
| 628 | 574.1 | 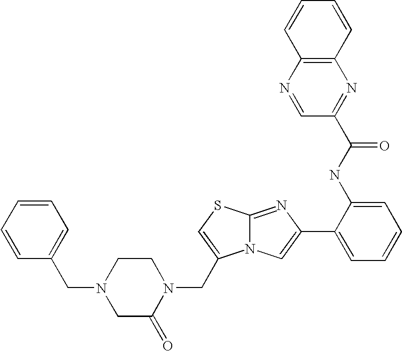 | | A′ | B |
| 629 | 429.0 | 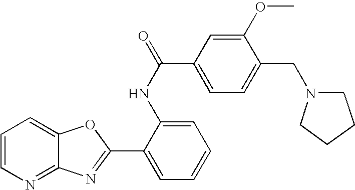 | | A | A |
| 630 | 403.1 | 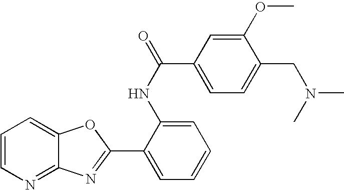 | | A | A |
| 631 | 445.0 | 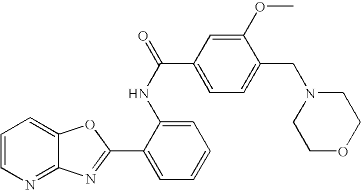 | | A′ | B |
| 632 | 458.1 | 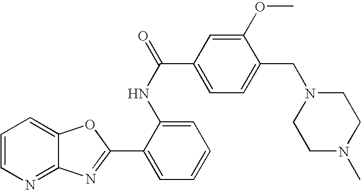 | | A | A |
| 633 | 423.3 | 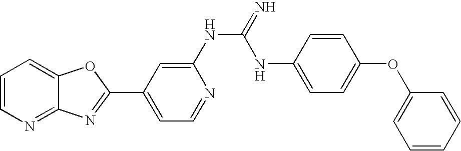 | | NA | C |
| 634 | 364.9 | 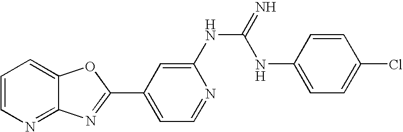 | | D | B |
| 635 | 421.1 | 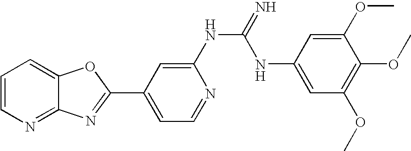 | | D | A |
| 636 | 359.9 | 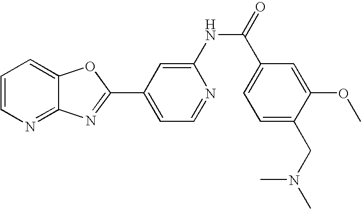 | | B | B |
| 637 | 459.3 | 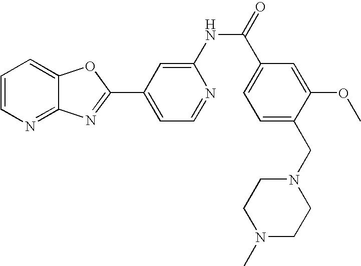 | | D | B |
| 638 | 445.2 | 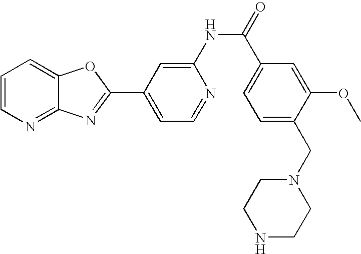 | | D | B |
| 639 | 378.9 | 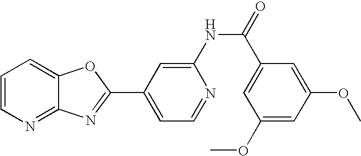 | | A | B |
| 640 | 368.9 | 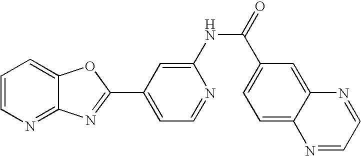 | | NA | C |
| 641 | 334.9 | 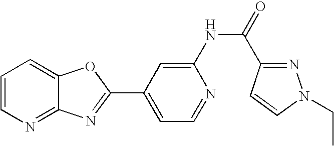 | | D | B |
| 642 | 446.2 | 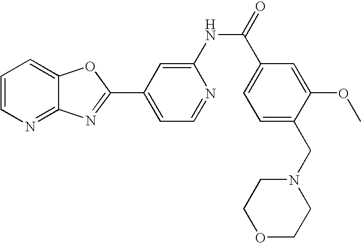 | | D | B |
| 643 | 535.1 | 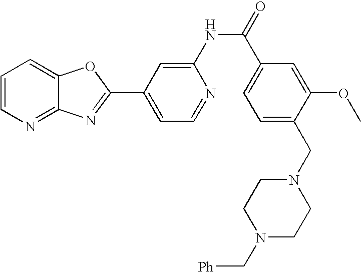 | | A | B |
| 644 | 434 | 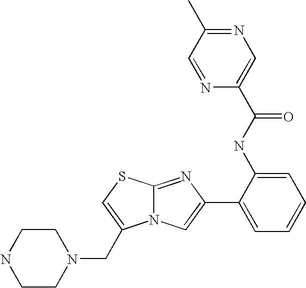 | | A | A |
| 645 | 469 | 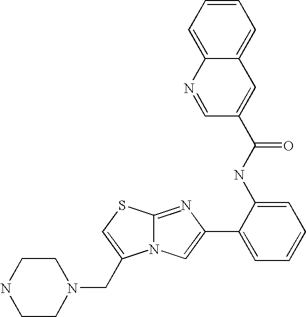 | | A′ | A |
| 646 | 481 | 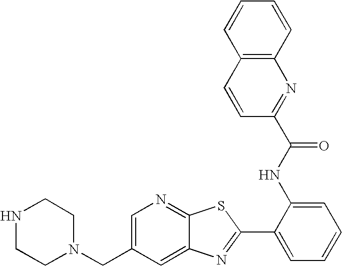 | | A | A |
| 647 | 473.1 | 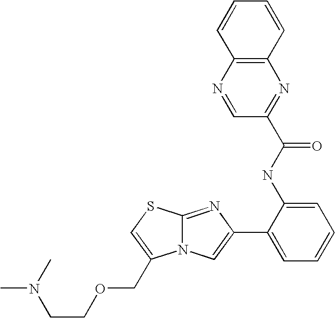 | | A′ | A |
| 648 | 402.1 | 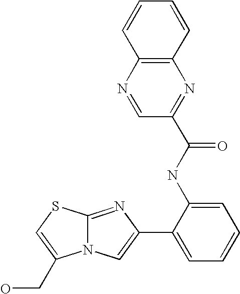 | | A′ | B |
| 649 | 512.2 | 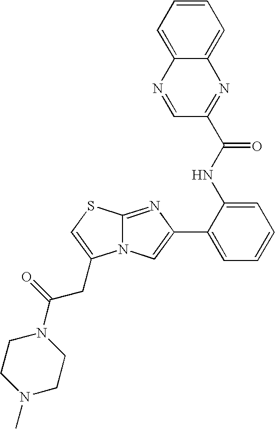 | | A′ | A |
| 650 | 570.2 | 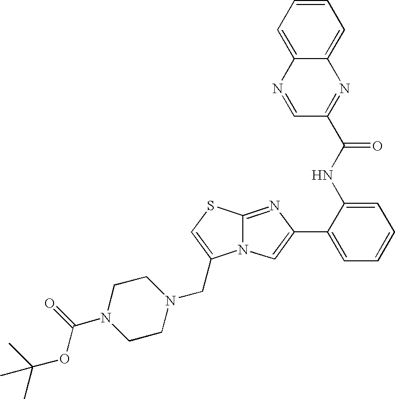 | | NA | |
| 651 | 512.2 | 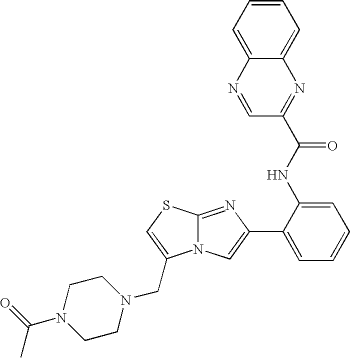 | | A′ | A |
| 655 | 393.0 | 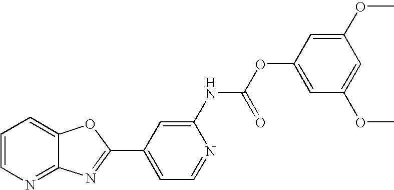 | | NA | C |
| 656 | 393.2 | 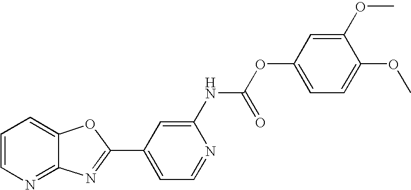 | | NA | C |
| 657 | 423.1 | 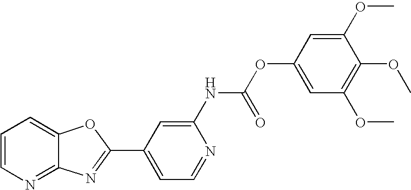 | | NA | C |
| 658 | 382.1 | 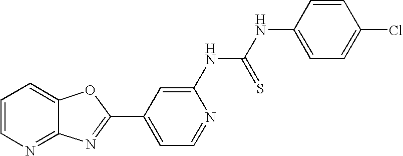 | | NA | C |
| 659 | 509.2 | 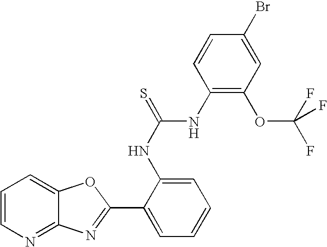 | | NA | C |
| 660 | 408.1 | 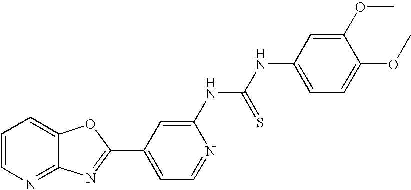 | | A | B |
| 661 | 408.2 | 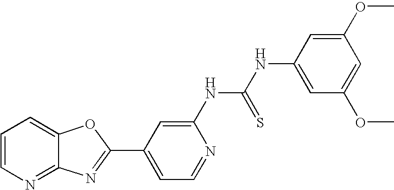 | | A′ | B |
| 662 | 378.2 | 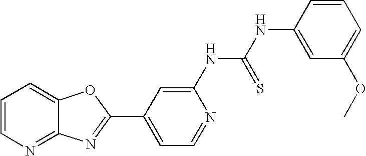 | | NA | C |
| 663 | 438.0 | 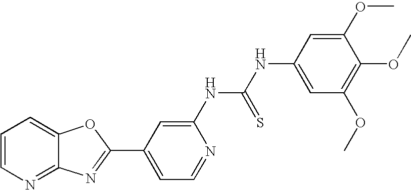 | | A | B |
| 664 | 477.8 | 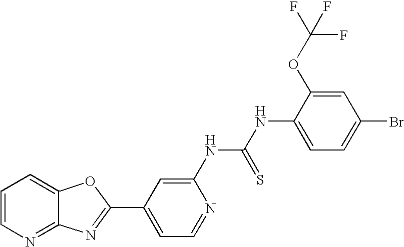 | | NA | C |
| 665 | 406.1 | 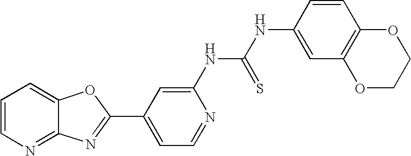 | | NA | C |
| 666 | 391.0 | 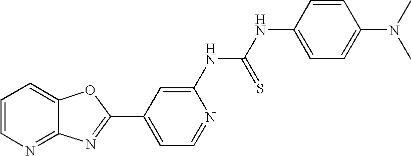 | | NA | C |
| 667 | 448.0 | 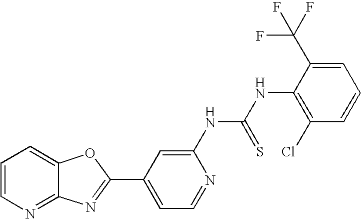 | | A | A |
| 668 | 410.0 | 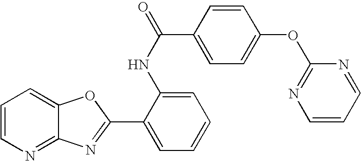 | | A | B |
| 669 | 392.0 | 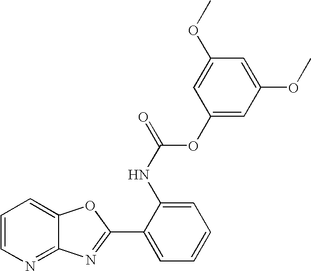 | | NA | C |
| 670 | 392.0 | 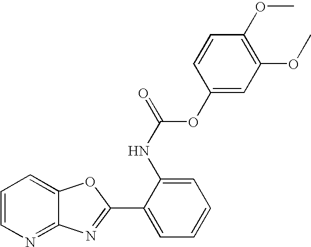 | | A | B |
| 671 | 422.0 | 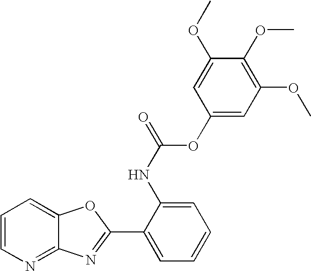 | | NA | C |
| 677 | | 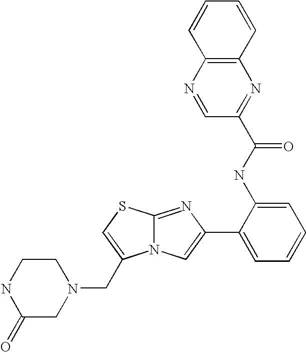 | | A′ | A |
| 678 | | 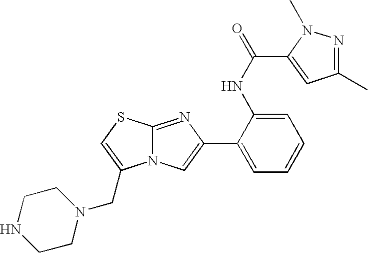 | | A | B |
| 679 | | 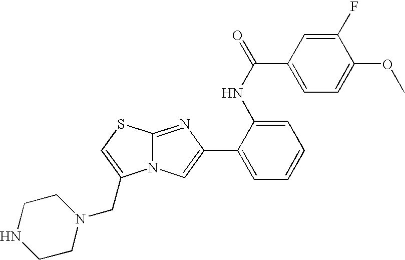 | | A | B |
| 680 | 484.2 | 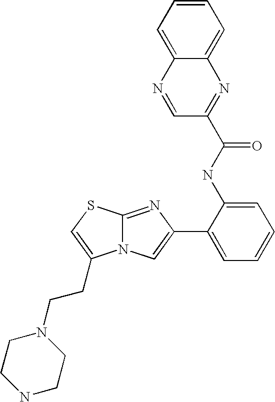 | | A′ | A |
| 681 | 522 | 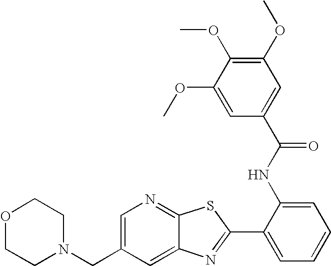 | | A | A |
| 672 | 415.1 | 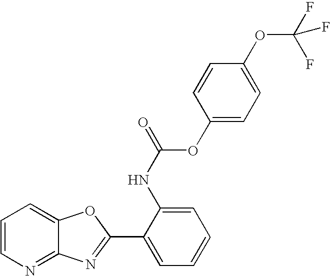 | | NA | C |
| 673 | | 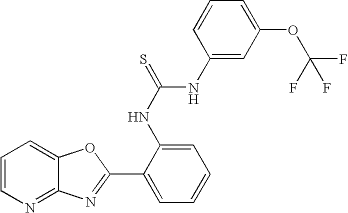 | | NA | C |
| 674 | | 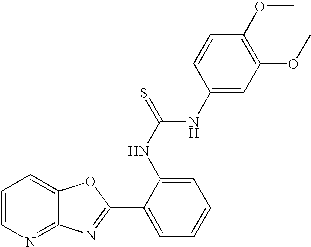 | | NA | C |
| 675 | 407.1 | 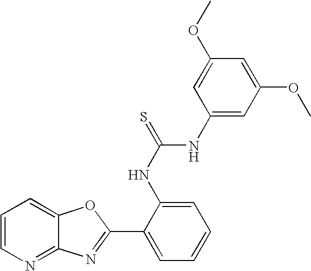 | | NA | C |
| 676 | | 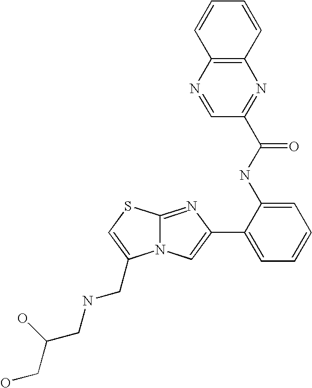 | | A′ | A |
| 682 | 492 | 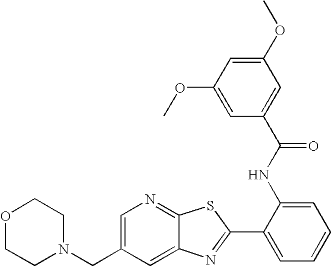 | | A′ | A |
| 683 | 475 | 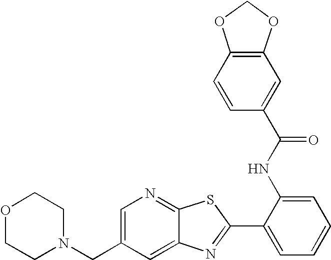 | | A | B |
| 684 | 460 | 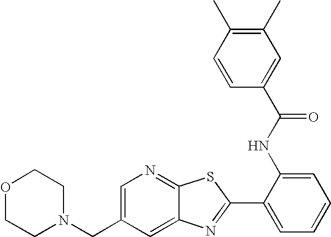 | | A | B |
| 685 | 456 | 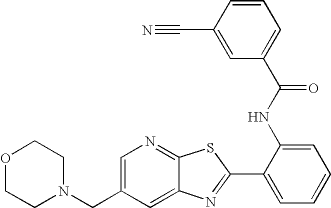 | | A′ | B |
| 686 | 475 | 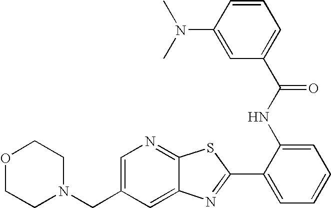 | | A′ | A |
| 687 | 467 | 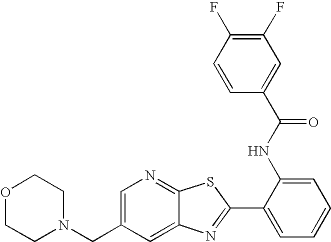 | | NA | C |
| 688 | 483 | 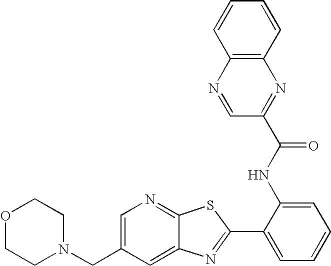 | | NA | C |
| 689 | 479 | 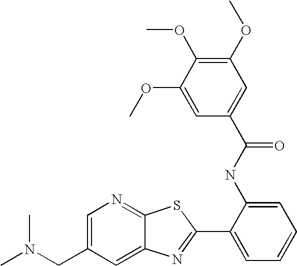 | | A | A |
| 690 | 483 | 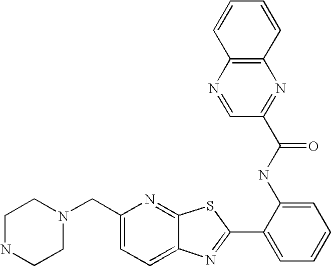 | | A′ | A |
| 692 | 469 | 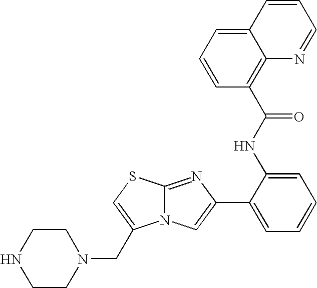 | | C | B |
| 695 | 454 | 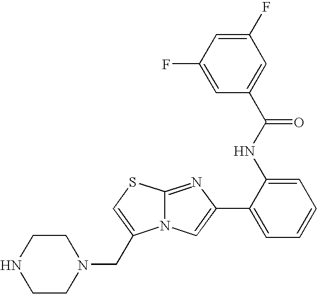 | | A′ | A |
| 697 | 596 | 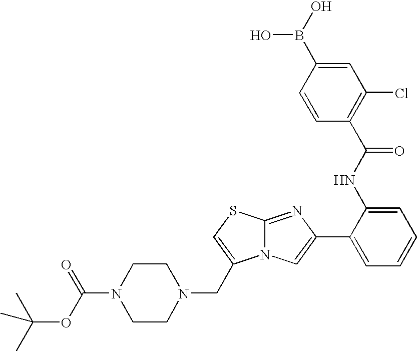 | | NA | C |
| 698 | 502 | 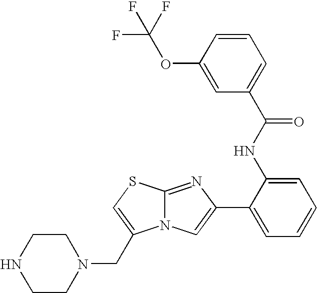 | | A′ | A |
| 699 | | 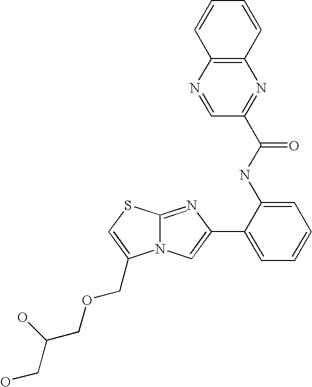 | | A | A |
| 700 | 512.2 | 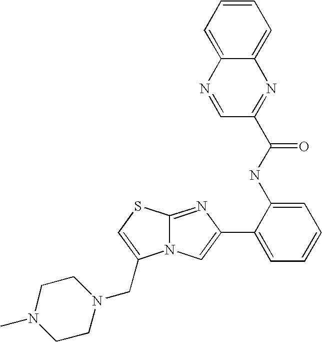 | | A′ | A |
| 701 | 477 | 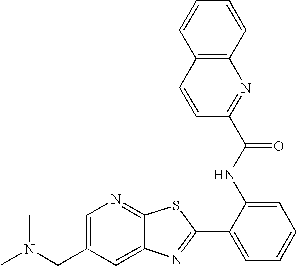 | | A′ | A |
| 702 | 534 | 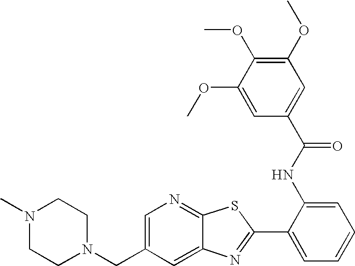 | | A | A |
| 703 | 468 | 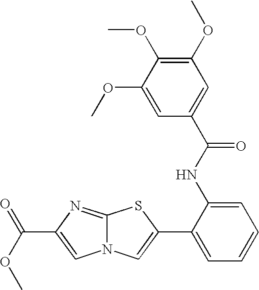 | | NA | C |
| 704 | 454 | 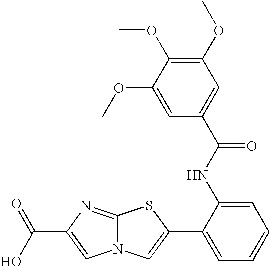 | | NA | C |
| 705 | 387 | 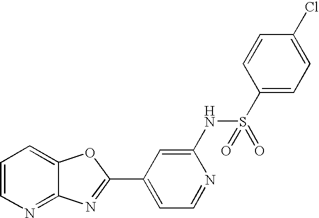 | | | |
| 706 | 445.1 | 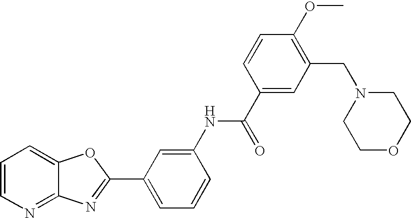 | | | |
| 707 | 386.1 | 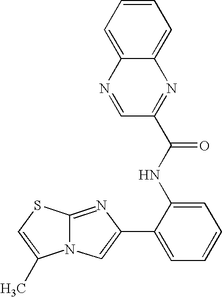 | | A′ | A |
| 708 | 494.2 | 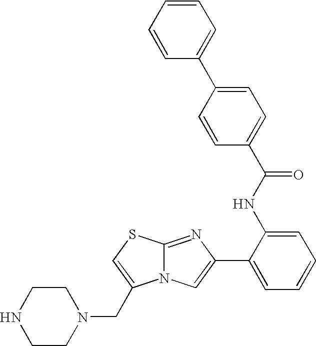 | | A′ | A |
| 709 | 494.1 | 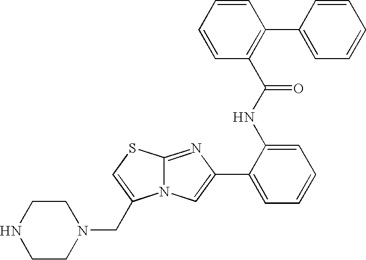 | | NA | C |
| 710 | 494.1 | 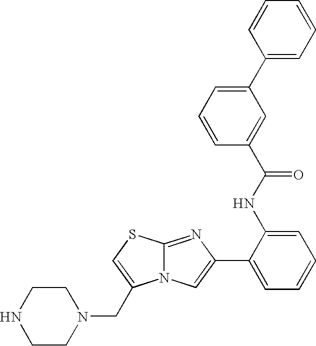 | | A′ | A |
| 711 | | 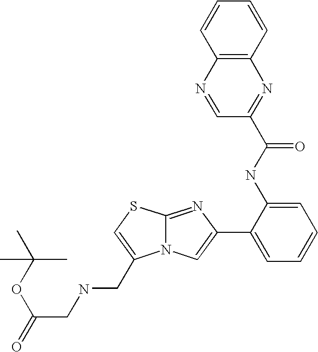 | | A | B |
| 714 | | 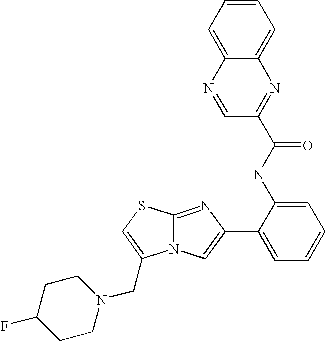 | | A | B |
| 715 | | 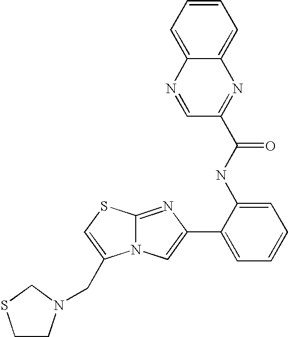 | | A′ | B |
| 716 | | 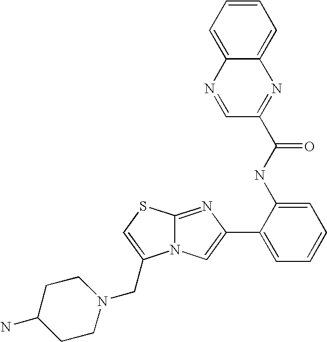 | | A′ | A |
| 717 | | 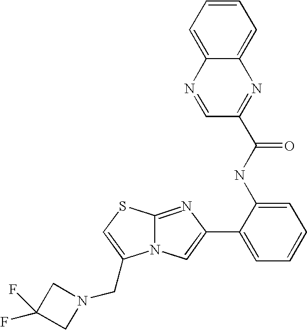 | | A | B |
| 718 | 444.1 | 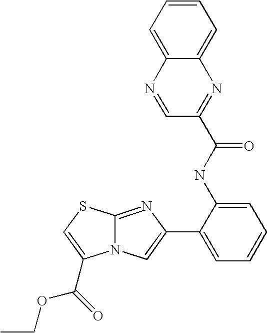 | | NA | C |
| 719 | 416 | 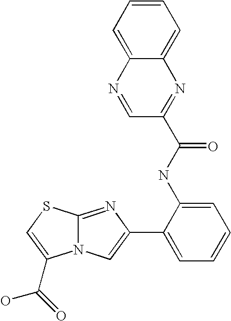 | | A | A |
| 720 | | 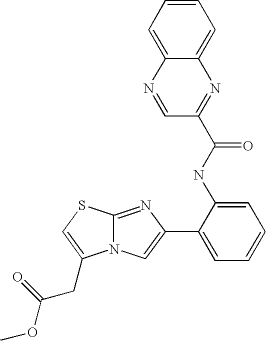 | | NA | C |
| 721 | | 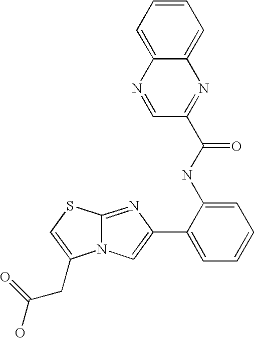 | | A | A |
| 722 | 449 | 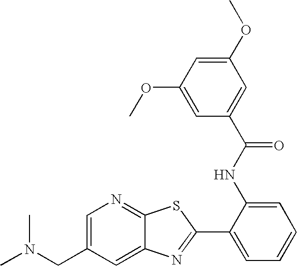 | | A′ | A |
| 723 | 490 | 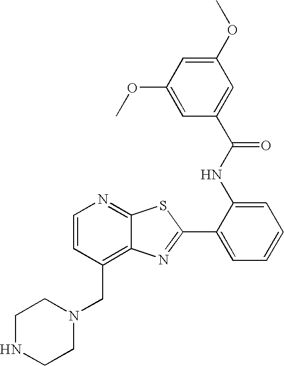 | | A | A |
| 724 | 482 | 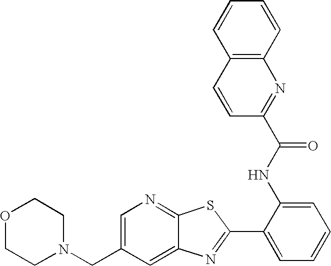 | | A′ | A |
| 725 | 505 | 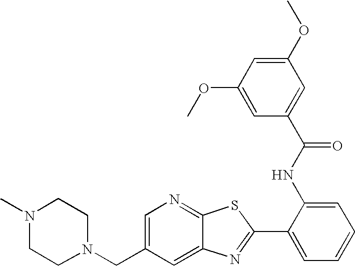 | | A′ | A |
| 726 | 491 | 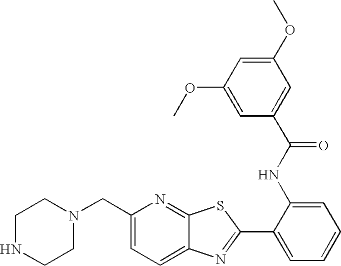 | | A′ | A |
| 727 | 458 | 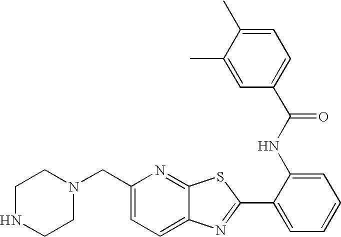 | | A′ | A |
| 728 | 466 | 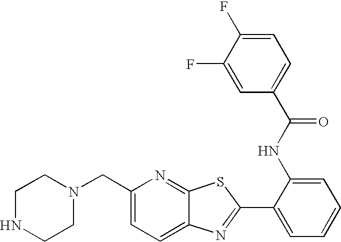 | | A | A |
| 729 | 481 | 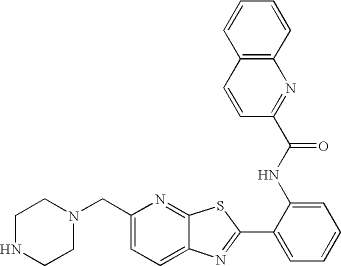 | | A′ | A |
| 730 | 488 | 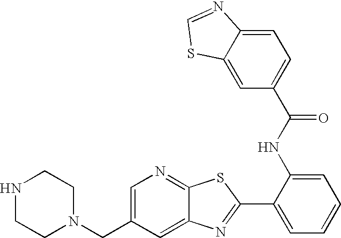 | | A | A |
| 731 | 498 | 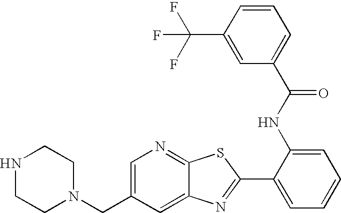 | | A | A |
| 732 | 497 | 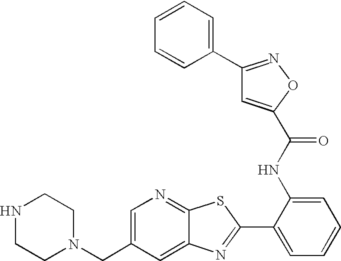 | | A′ | B |
| 733 | 484.2 | 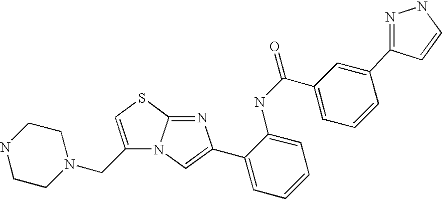 | | A′ | A |
| 735 | 514.2 | 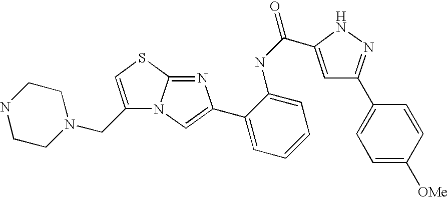 | | A′ | A |
| 736 | 514.2 | 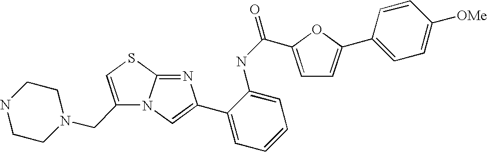 | | A′ | A |
| 737 | 500.1 | 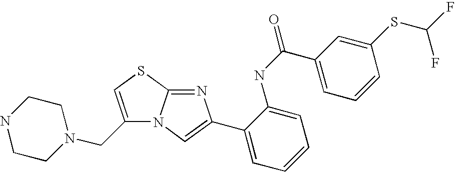 | | A′ | A |
| 738 | 448.1 | 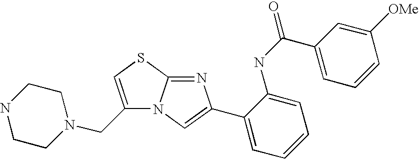 | | A | A |
| 739 | 424.1 | 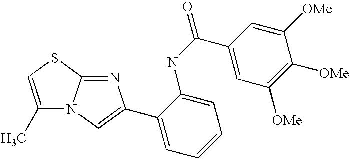 | | A′ | A |
| 740 | 377.1 | 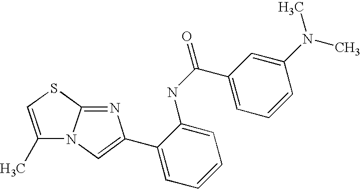 | | A′ | B |
| 741 | 498.1 | 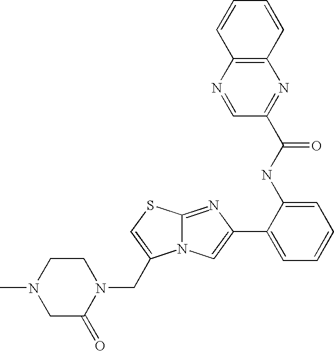 | | A′ | A |
| 742 | 487.1 | 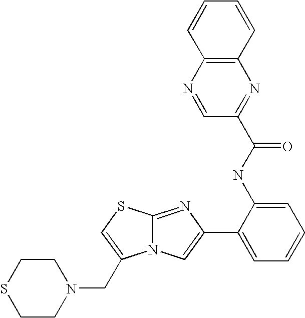 | | A′ | B |
| 743 | 466.1 | 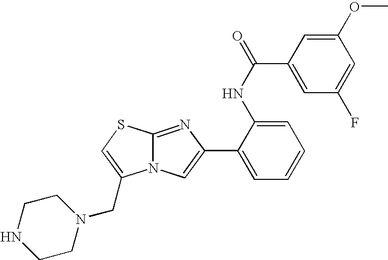 | | A | A |
| 744 | 437.1 | 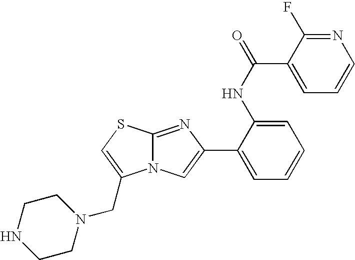 | | B | A |
| 745 | 452.1 | 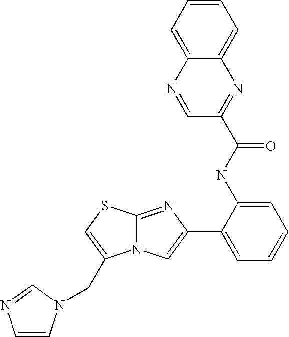 | | A′ | A |

| COM- | | | | |
|---|---|---|---|---|
| POUND | | | IC₅₀ FP | IC₅₀ MS |
| NO | [M + H]+ | STRUCTURE | ASSAY | ASSAY |
| 13 | | 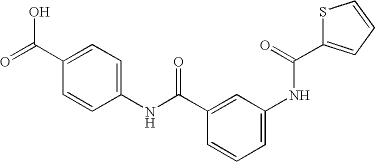 | A | |
| 14 | | 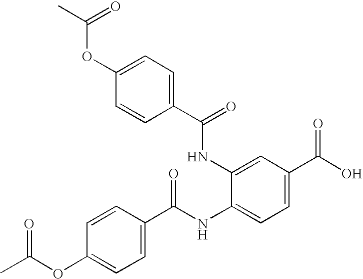 | B | |
| 15 | | 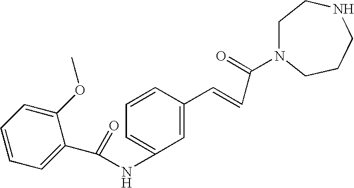 | C | |
| 23 | 359.4 | 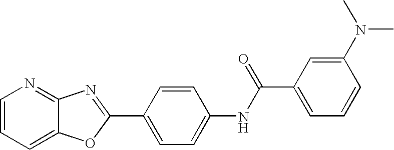 | D | |
| 25 | 341.3 | 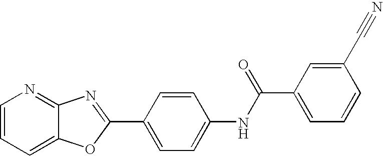 | B | |
| 26 | 341.4 | 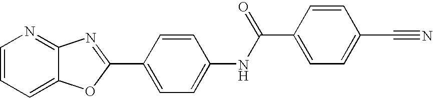 | B | |
| 28 | 401.1 | 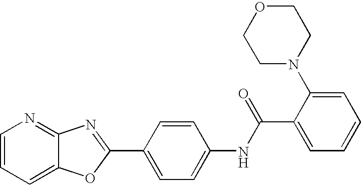 | D | |
| 30 | 380.0 | 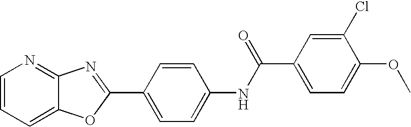 | D | |
| 44 | 341.0 | 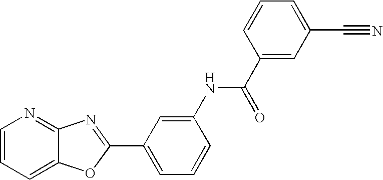 | D | |

TABLE 6

| COMPOUND NO | STRUCTURE | ED₅₀ |
|---|---|---|
| 11 | 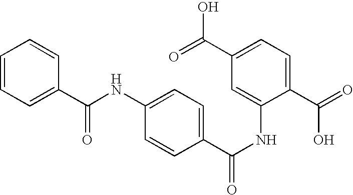 | N/A |
| 12 | 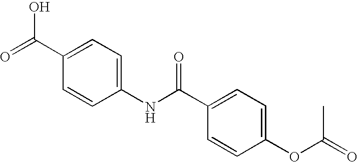 | N/A |

TABLE 8

| COMPOUND | | ED₅₀ ATP | IC₅₀ ATP |
|---|---|---|---|
| NO | STRUCTURE | ASSAY | ASSAY |
| 52 | 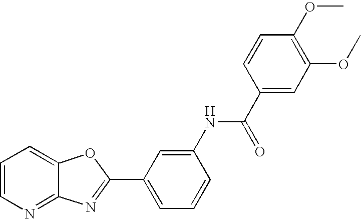 | A | |
| 42 | 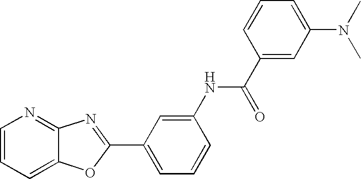 | A | |
| 49 | 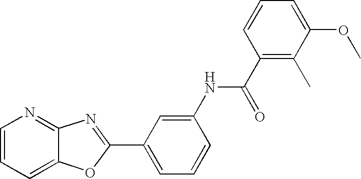 | A | |
| 115 | 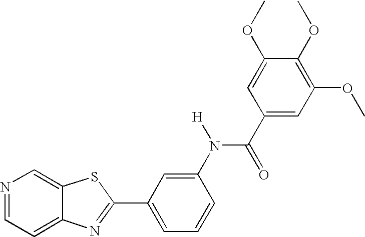 | D | |
| 79 | 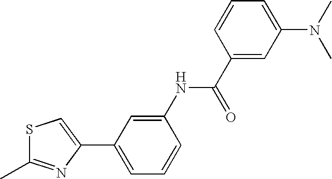 | A | |
| 117 | 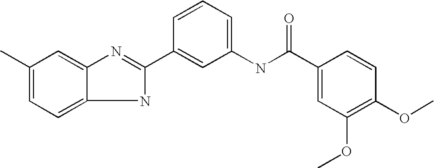 | | B |
| 120 | 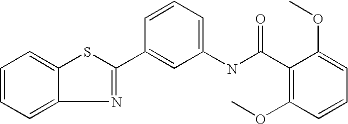 | A | |
| 121 | 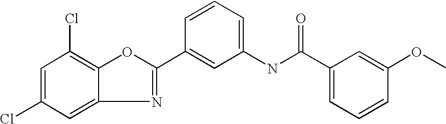 | NA | |
| 123 | 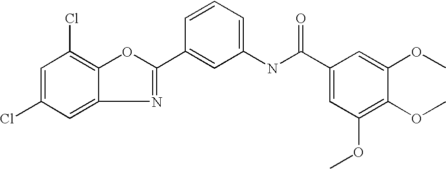 | D | |
| 85 | 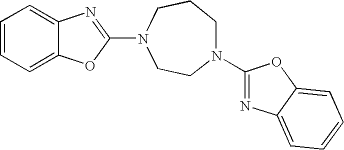 | NA | |
| 86 | 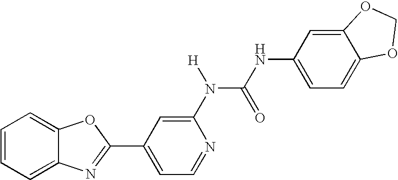 | A | |
| 87 | 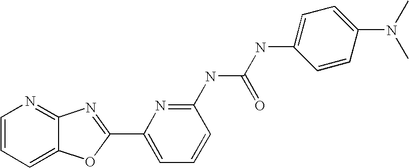 | NA | |
| 88 | 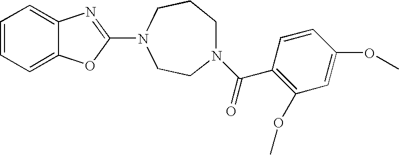 | NA | |
| 89 | 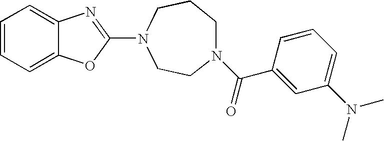 | | A |
| 90 | 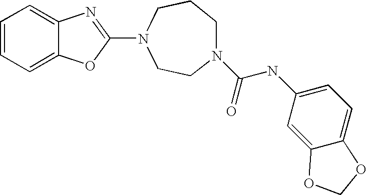 | D | |
| 91 | 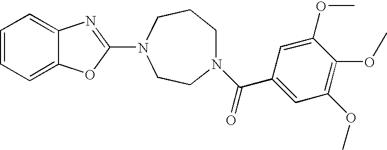 | | A |
| 92 | 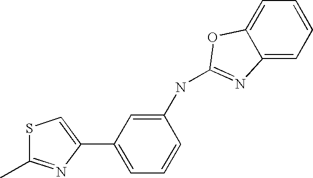 | | B |
| 93 | 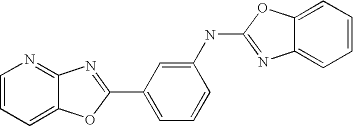 | D | |
| 94 | 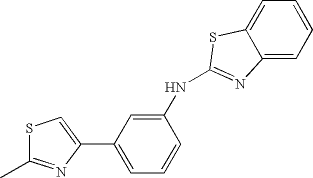 | D | |
| 95 | 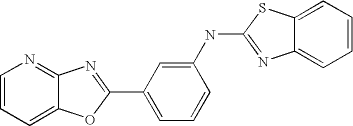 | NA | |
| 97 | 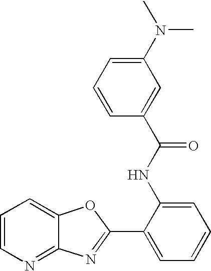 | A | |
| 98 | 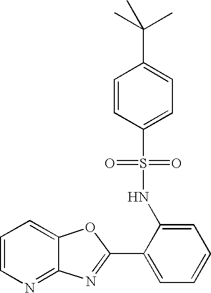 | NA | |
| 99 | 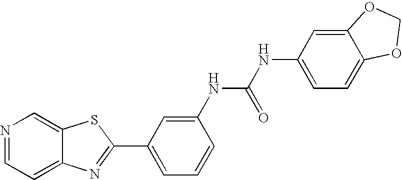 | A | |
| 100 | 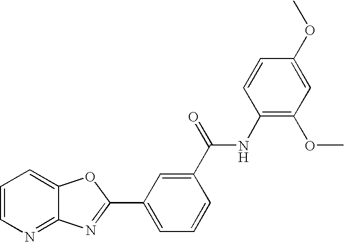 | A | |
| 101 | 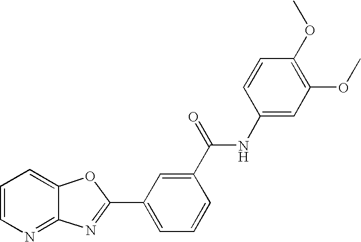 | C | |
| 102 | 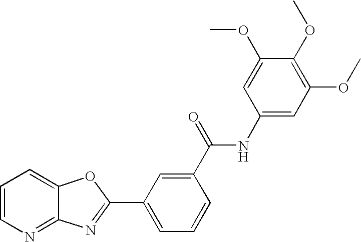 | A | |
| 103 | 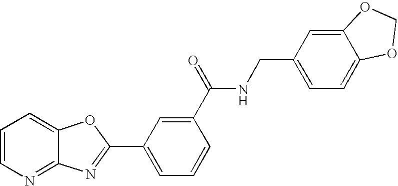 | NA | |
| 104 | 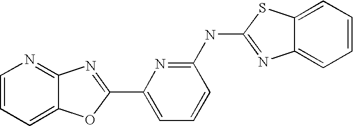 | A | |
| 105 | 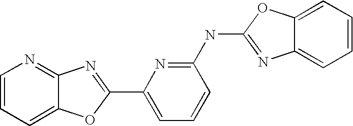 | A | |
| 133 | 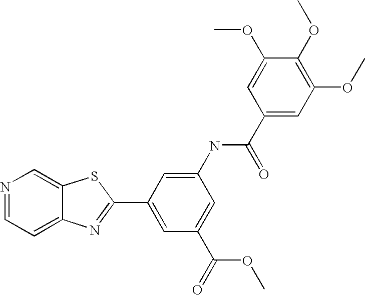 | NA | |
| 134 | 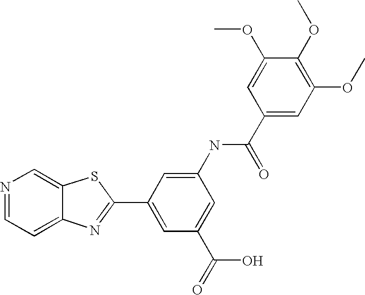 | NA | |

Modulators

In one embodiment, [symbol] modulating compounds of the invention are represented by Structural Formula (I):

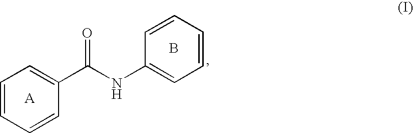

or a salt thereof, where:

Ring A is optionally substituted; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In certain embodiments, Ring B is substituted with at least a carboxy group.

In certain embodiments, Ring B is substituted with at least a substituted or unsubstituted arylcarboxamine, a substituted or unsubstituted aralkylcarboxamine or a polycyclic aryl group.

In certain embodiments, Ring B is substituted with at least a substituted or unsubstituted heteroaryl group or a substituted or unsubstituted heterocyclylcarbonylethenyl group.

In another embodiment, [symbol] modulating compounds of the invention are represented by Structural Formula (II):

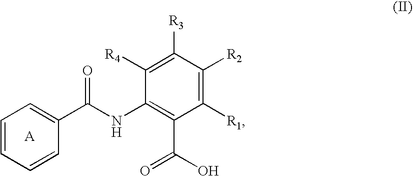

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In a further embodiment, [symbol] -modulating compounds of the invention are represented by Structural Formula (IIa):

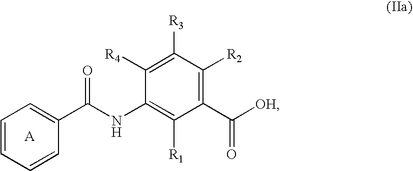

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₄, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In yet another embodiment, [symbol] -modulating compounds of the invention are represented by Structural Formula (II):

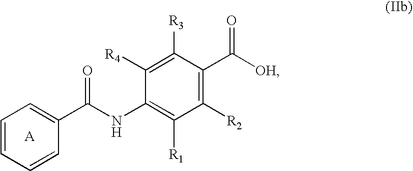

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)R₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ in Structural Formulas (II)-(IIb) are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention provides novel [symbol] -modulating compounds of Formula (III):

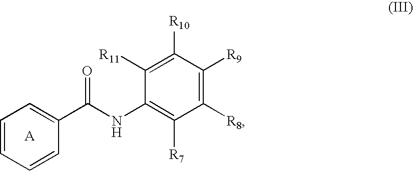

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group; particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In certain such embodiments, R₈ is selected from thiazolopyridyl, imidazothiazolyl, benzoxazinonyl, or imidazopyridyl.

Particular examples of R₈, where

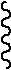

indicates attachment to the remainder of Structural Formula (III), include:

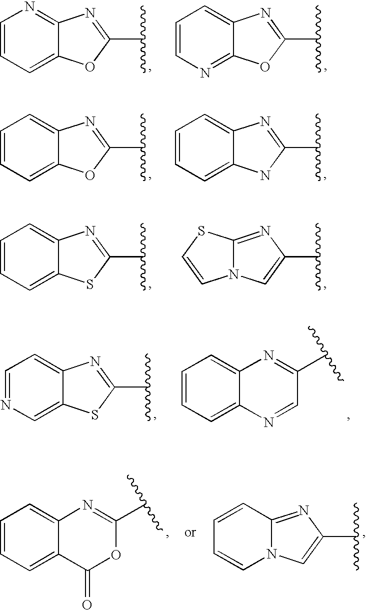

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

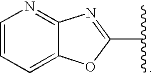

In certain embodiments, R₈ is

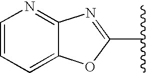

and Ring A is optionally substituted with up to 3 substituents independently selected from (C₁-C₃ straight or branched alkyl), O—(C₁-C₃ straight or branched alkyl), N(C₁-C₃ straight or branched alkyl), halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

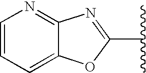

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

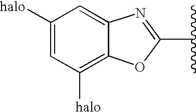

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments R₈ is selected from

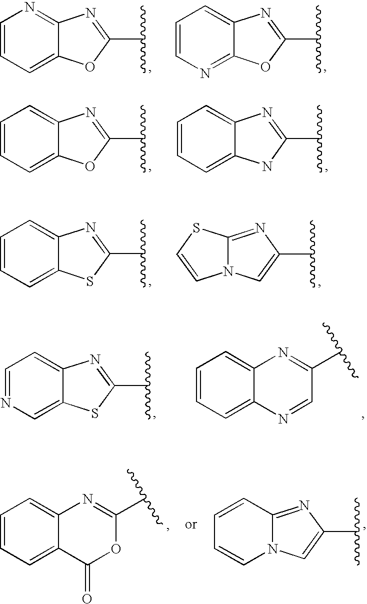

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

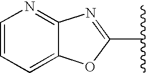

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

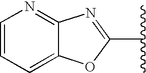

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In yet another aspect, the invention provides novel [symbol] modulating compounds of Formula (IV):

Ar-L-J-M-K-Ar′ (IV)

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂′C(O)NR₂′R₂′; NR₂′C(O)C(O)R₂′; NR₂′C(O)R₂′; NR₂′(COOR₂′); NR₂′C(O)R₅′; NR₂′S(O)₂NR₂′R₂′; NR₂′S(O)₂R₂′; NR₂′S(O)₂R₅′; NR₂′C(O)C(O)NR₂′R₂′; NR₂′C(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₇′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur, oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In a preferred embodiment of the invention, each Ar, L, and Ar′ is independently an optionally substituted 5- to 7-membered monocyclic ring system or an optionally substituted 9- to 12-membered bicyclic ring system.

According to another preferred embodiment,
Ar is

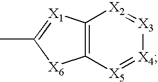

X₁, X₂, X₃, X₄, and Xs are independently selected from CR₁′ and N; and
X₆ is selected from NR₁′, O, and S;

According to yet another preferred embodiment, X₁ and X₂ are N; X₃, X₄, and X₅ are CR₁′; and X₆ is O.

According to still yet another preferred embodiment, X₁ and X₃ are N; X₂, X₄, and X₅ are CR₁′; and X₆ is O.

According to still yet another preferred embodiment, X₁ and X₄ are N; X₂, X₃, and X₅ are CR₁′; and X₆ is O.

According to still yet another preferred embodiment, X₁ and X₅ are N; X₂, X₃, and X₄ are CR₁′; and X₆ is O.

In another embodiment, the compounds of the formula above are those wherein J is NR₁′, K is absent, and M is C(O).

In yet another embodiment, the compounds of the formula above are those wherein J is absent, K is NR₁′, and M is C(O).

In a further embodiment, compounds of formula (IV) e those where when J is absent and K is NR₁′, M is not C(O) and when J is NR₁′ and K is absent, M is not C(O).

In a preferred embodiment, the compounds above are those wherein L is an optionally substituted 5- to 7-membered carbocyclic or heterocyclic aryl group.

In yet another preferred embodiment, the compounds are those wherein L is an optionally substituted phenylene, pyridinylene, imidazolylene, oxazolylene, or thiazolylene.

In a particularly preferred embodiment, L is an optionally substituted phenylene.

In another particularly preferred embodiment, L is an optionally substituted pyridinylene.

In an even more preferred embodiment, L is phenylene.

In another even more preferred embodiment, L is pyridinylene.

In either of these embodiments, Ar and J may be attached to L at the ortho-, meta-, or para-positions. Particularly preferred are those embodiments where attachment is at the meta-position.

In certain embodiments, L is not phenylene when Ar′ is phenyl. Examples of such embodiments include embodiments where L is an optionally substituted heterocyclic aryl group and Ar′ is an optionally substituted carbocyclic or heterocyclic aryl group, or wherein L is an optionally substituted carbocyclic or heterocyclic aryl group and Ar′ is an optionally substituted heterocyclic aryl group.

In yet another aspect, the invention provides novel sirtuin-modulating compounds of Formula (I) or a salt thereof, wherein

Ring A is substituted with at least one R₁′ group;

R₁′, R₂′, R₃′, R₄′, R₅′, R₆′, R₇′, R₈′, R₉′, R₁₀′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group;

each aryl is independently a 5- to 7-membered monocyclic ring system or a 9- to 12-membered bicyclic ring system optionally substituted with 1-3 independent C1-alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₉′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′; and

Ring B is substituted with at least one

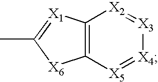

wherein

X₁, X₂, X₃, X₄, and X₅ are independently selected from CR₁′ and N; and

X₆ is selected from NR₁′, O, and S.

In a preferred embodiment, Ring B is phenyl or pyridinyl.

In a further aspect, the invention provides novel [symbol] -modulating compounds of Formula (IVa):

Het-L-Q-Ar′ (IVa)

or a salt thereof, wherein:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—,

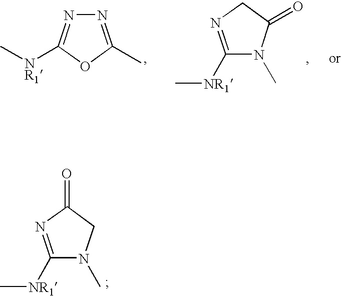

and

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In certain embodiments, when Het is a polycyclic heteroaryl, L is optionally substituted phenylene, and Ar′ is optionally substituted phenyl; then Q is not —NH—C(O)—.

In certain embodiments, Het and Q are attached to L in a 1-, 2- or 1-,3-configuration (e.g., when L is phenylene, Het and Q are attached in an ortho or a meta orientation). In certain embodiments where Het and Q are attached to L in a 1-,3-configuration, if Het is benzoxazolyl, L is pyridylene and Q is —NH—C(O)—NH, then Ar′ is not 3,4 dioxymethlyene phenyl; if Het is methyl thiazolyl, L is phenylene and Q is —NH—C(O)—, then Ar′ is not 3-dimethylamino phenyl; if Het is oxazolopyridyl, L is pyridylene and Q is —NH—C(O)—NH, then Ar′ is not 4-dimethylamino phenyl; if Het is oxazolopyridyl or benzoxazolyl and L is

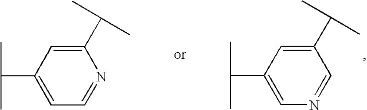

then Q is not —NH—(SO)₂—; and if Het is oxazolopyridyl, L is

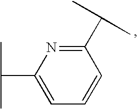

and Q is —NH—C(O)—, then Ar′ is not 3,4 dimethoxyphenyl or pyridyl.

When Het is substituted, it is typically substituted at up to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

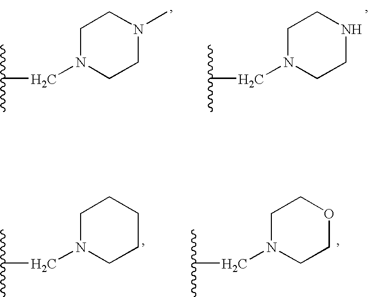

where each R₁₂ is independently selected from optionally substituted C₁-C₃ straight or branched alkyl.

In certain embodiments, Het is selected from oxazolopyridyl, benzothienyl, benzofuryl, indolyl, quinoxalinyl, benzothiazolyl, benzoxazolyl, benzimidazolyl, quinolinyl, isoquinolinyl or isoindolyl. In other embodiments, Het comprises one ring N heteroatom and 1 to 2 additional ring heteroatoms independently selected from N, O or S, such as thiazolyl, triazolyl, oxadiazolyl, thiazolopyridyl, imidazothiazolyl, benzoxazinonyl, or imidazopyridyl.

Particular examples of Het include:

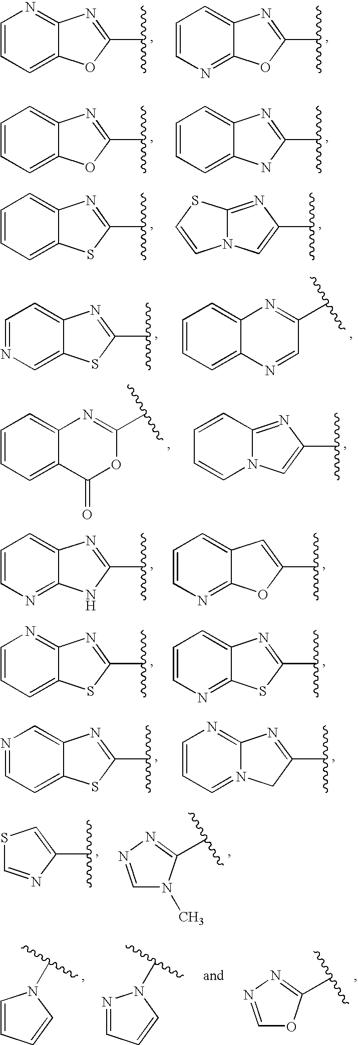

where up to 2 ring carbons not immediately adjacent to the indicated attachment point are independently substituted with optionally substituted C₁-C₃ straight or branched alkyl, phenyl, halo, N(R₁₂)₂, NH(R₁₂), OR₂, C(O)—NH—R₁₂, C(O)—N(R₁₂)₂, N(R₁₂)—OR₁₂, CH₂—N(R₁₂)₂, C(O)OR₁₂, C(O)OH,

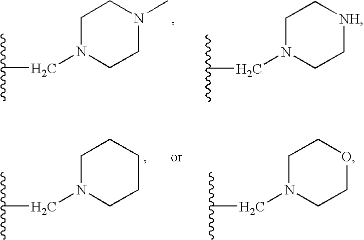

wherein each R₁₂ is independently selected from optionally substituted C₁-C₃ straight or branched alkyl.

In certain embodiments, L is selected from

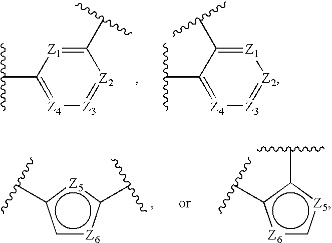

wherein:

each of Z₁, Z₂, Z₃ and Z₄ is independently selected from CH or N, wherein not more than three of said Z₁, Z₂, Z₃ or Z₄ is N;

each of Z₅ and Z₆ is independently selected from C, N, O or S, provided that at least one of Zs and Z₆ is N; and

L is optionally substituted at 1 to 2 carbon atoms with a substituent independently selected from R₁₂, N(R₁₂)₂, NH(R₁₂), OR₁₂, C(O)—NH—R₁₂, C(O)—N(R₁₂)₂, N(R₁₂)—OR₁₂, CH₂—N(R₁₂)₂, C(O)OR₁₂, C(O)OH,

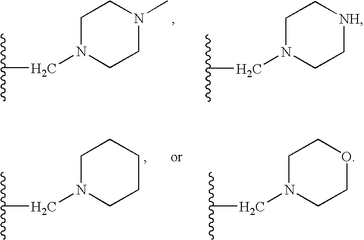

In preferred embodiments, L is selected from phenylene or pyridylene, such as unsubstituted phenylene or phenylene substituted with a single substituent selected from C(O)OCH₃, C(O)OH, CH₂OH, N(CH₃)₂, or CH₂N(CH₃)₂, or unsubstituted pyridylene.

In certain embodiments, Q is selected from —NH—C(O)—, —NH—S(O)ᵣ, —NH—C(O)—NH—, —C(O)—NH—, —CH₂—, —N(CH₃)—C(O)—NH—, —NH—C(O)—N(CH₃)—, or —NH—S(O)₂—NH—, particularly —NH—C(O)—, —C(O)—NH—, —NH—, —NH—C(O)—NH, or —NH—S(O)₂—.

In certain embodiments, Ar′ is selected from optionally substituted phenyl, benzothiazolyl, or benzoxazolyl. When Ar′ is phenyl, typical optional substituents are 1 to 3 substituents independently selected from halo, (optionally substituted C₁-C₃ straight or branched alkyl), O-(optionally substituted C₁-C₃ straight or branched alkyl), S-(optionally substituted C₁-C₃ straight or branched alkyl), N(CH₃)₂ or optionally substituted heterocyclyl, or wherein two substituents on adjacent ring atoms are taken together to form a dioxymethylene.

In certain embodiments, Het is selected from

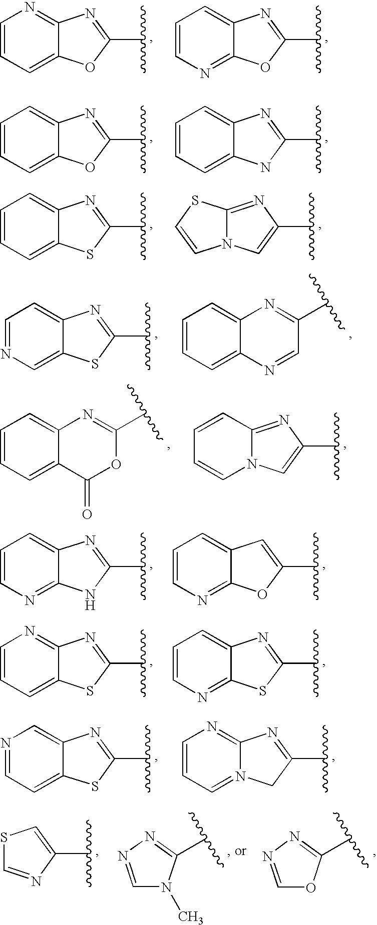

and wherein up to 2 ring carbons not immediately adjacent to the indicated attachment point are independently substituted with optionally substituted C₁-C₃ straight or branched alkyl, phenyl or halo;

L is selected from unsubstituted phenylene, phenylene substituted with a single substituent selected from C(O)OCH₃, C(O)OH, CH₂OH, N(CH₃)₂, or CH₂N(CH₃)₂, or unsubstituted pyridylene;

Q is selected from —NH—C(O)—, —C(O)—NH—, —NH—, —NH—C(O)—NH, or —NH—S(O)₂—; and

Ar′ is selected from optionally substituted phenyl, benzothiazolyl, or benzoxazolyl, wherein said phenyl is optionally substituted with 1 to 3 substituents independently selected from chloro, methyl, O-methyl, S-methyl, N(CH₃)₂, morpholino, or 3,4 dioxymethylene.

In certain embodiments, Q is selected from —NH—C(O)—, —C(O)—NH—, —NH— or —NH—C(O)—NH.

In certain embodiments, the substituents on Ar′ are selected from chloro, methyl, O-methyl, S-methyl or N(CH₃)₂. In certain embodiments, the only substituent on Ar′ is an O-methyl group, particularly an O-methyl group ortho or meta to Q. In certain embodiments, when there are two or more O-methyl groups or Ar′, at least one is ortho or meta to Q.

In certain embodiments, L is pyridyl and Het and Q are at the 1,3- or 2,4-position with respect to the pyridyl nitrogen atom. In certain such embodiments, Q is —NH—S(O)₂—.

In certain embodiments where L is further substituted, the substituent is typically meta to both Het and Q.

In certain embodiments, Q is —NH— and Het is thiazolyl or oxazolopyridyl.

In certain embodiments, Q is —NH— and Ar is benzothiazolyl or benzoxazolyl.

In certain embodiments,

L is

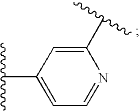

and Q is —NH—(SO)₂—. In certain such embodiments, Het is oxazolopyridyl. When L, Q and optionally Het have these values, Ar′ is advantageously naphthyl or phenyl, where Ar′ is optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments,

L is

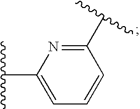

and Q is —NH—C(O)—. In certain such embodiments. Het is oxazolopyridyl. When L, Q and optionally Het have these values, Ar′ is advantageously pyridyl or phenyl optionally substituted with 1 to 3 substituents independently selected from CN, halo, (C₁-C₃ straight or branched alkyl), O—(C₁-C₃ straight or branched alkyl), N(C₁-C₃ straight or branched alkyl)₂, or a 5 to 6-membered heterocycle.

In certain embodiments,

[symbol] . Het comprises one N heteroatom and 1 to 2 additional heteroatoms independently selected from N, O or S;

L is

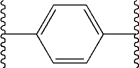

and is optionally substituted;

Q is —NH—C(O)—; and

Ar′ is phenyl substituted with 1 to 3 substituents independently selected from CN, halo, C₁-C₃ straight or branched alkyl, O—(C₁-C₃ straight or branched alkyl), N(C₁-C₃ straight or branched alkyl)₂, or a 5 to 6-membered heterocycle, wherein when R₈ is unsubstituted

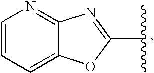

then ring A is:
a) not simultaneously substituted at the 2- and 6-positions with O—(C₁-C₃ straight or branched alkyl);
b) not simultaneously substituted at the 2-position with C₁-C₃ straight or branched alkyl or O—(C₁-C₃ straight or branched alkyl) and at the 3-position with O—(C₁-C₃ straight or branched alkyl);
c) not substituted at the 4-position with O—(C₁-C₃ straight or branched alkyl) unless simultaneously substituted at the 3-position with halo or O—(C₁-C₃ straight or branched alkyl) and unsubstituted at all other positions;
   not substituted at the 4-position with N(C₁-C₃ straight or branched alkyl)₂, or said 5 to 6-membered heterocycle. In certain such embodiments, L is unsubstituted and/or Het is oxazolopyridyl:

In yet another aspect, the invention provides novel [symbol] -modulating compounds of Formula (V):

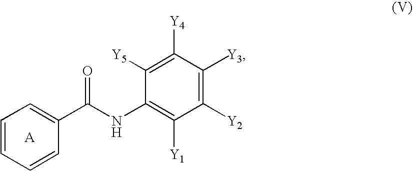

or a salt thereof, wherein:

Ring A is optionally substituted with at least one R₁′ group;

Y₁, Y₂, Y₃, Y₄, and Y₅ are independently R₁′;

R₁′, R₂′, R₃′, R₄′, R₅′, R₆′, R₇′, R₈′, R₉′, R₁₁′, and R₁₁′ are as defined above;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉′C(O)NR₉′R₉′; NR₉′C(O)C(O)R₉′; NR₉′C(O)R₆′; NR₉′S(O)₂NR₉′R₉′; NR₉′S(O)₂R₆′; NR₉′C(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉, COOR₉′, NO₂, CN, C(O)R₉′, C(O)NR₉′R₉′, NHC(O)R₉′, NH(COOR₉′), S(O)₂NR₉′R₉, OC(O)R₉′; or R₉′.

In a preferred embodiment of the above compound,

either Y₂ or Y₃ is

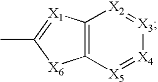

X₁, X₂, X₃, X₄, and X₅ are independently selected from CR₁′ and N; and

X₆ is selected from NR₁′, O, and S.

According to an even more preferred embodiment, X₁ and X₂ are N; X₃, X₄, and X₅ are CR₁′; and X₆ is O.

According to another even more preferred embodiment; X₁ and X₃ are N; X₂, X₄, and X₅ are CR₁′; and X₆ is Q.

According to another even more preferred embodiment, X₁ and X₄ are N; X₂, X₃, and X₅ are CR₁′; and X₆ is O.

According to another even more preferred embodiment, X₁ and Xs are N; X₂, X₃, and X₄ are CR₁′; and X₆ is O.

In another aspect, the invention provides [symbol] -modulating compounds of Structural Formula (VII):

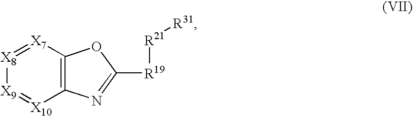

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²¹ or CR₁′; and
zero to one R²⁰ is a solubilizing group;

R¹⁹ is selected from:

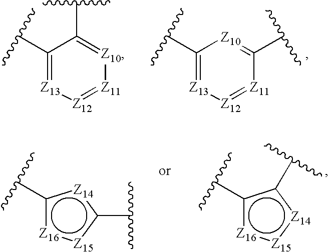

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that said compound is not:

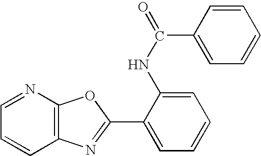

or
that when R¹⁹ is

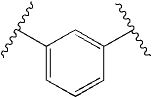

and R²⁷ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

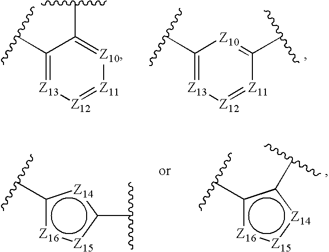

wherein:
each Z₁₀, Z₁₁, Z₁₂ and Z₁₃ is independently selected from N, CR₂₀, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that: said compound is not:

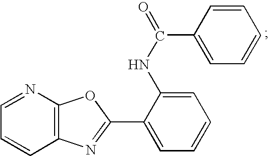

and

when X₈ and X₉ are each independently selected from CR²⁰ or CR₁′, R¹⁹ is

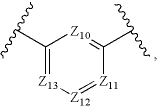

and each of Z₁₀, Z₁₁, Z₁₂ and Z₁₃ is independently selected from CR²⁰, or CR₁′, then:

a) at least one of Xs and X₉ is not CH; or

b) at least one of Z₁₀, Z₁₁, Z₁₂ and Z₁₃ is CR²⁰, wherein R²⁰ is a solubilizing group.

In certain embodiments, when Z₁₂ is CR²⁰ and R²⁰ is a solubilizing group, the solubilizing group is not —C(O)OCH₂CH₃, —COOH,

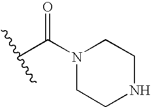

or

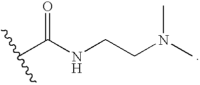

In certain embodiments, when X₈ and X₉ are each independently selected from CR²⁰ or CR₁′, R¹⁹ is

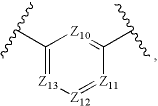

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁ and Z₁₃ is CR²⁰, wherein R²⁰ is a solubilizing group.

In certain embodiments, when R¹⁹ is

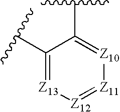

and each of Z₁₀, Z₁₁, Z₁₂ and Z₁₃ is CR²⁰, or CR₁′; X₈ and X₉ are CR²⁰ or CR₁′; R²¹ is —NHC(O)— and R³¹ is optionally substituted phenyl, then R³¹ is a substituted phenyl, at least one R₁′ in a CR₁′ moiety is optionally substituted C₁-C₃ straight or branched alkyl, or at least one R²⁰ in a CR²⁰ is a solubilizing group, or a combination thereof.

In certain embodiments, R¹⁹ is selected from phenyl, pyridyl, thienyl or furyl.

In certain embodiments, R¹⁹ is

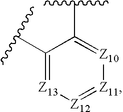

wherein each of Z₁₀, Z₁₁, Z₁₂ and Z₁₃ is independently selected from CR²⁰ or CR₁′; and

R²¹ is —NH—C(O)—; and

R³¹ is a substituted phenyl.

In certain such embodiments, when X₉ is N, R³¹ is not 2,4 dimethoxyphenyl and/or when X₁₀ is N, R³¹ is not halo substituted phenyl; 3,4-dioxoethylenephenyl; or 3,5-dimethoxyphenyl.

In preferred embodiments, R³¹ is optionally substituted with 1 to 3 substituents independently selected from —OCH₃, —CH₃, —N(CH₃)₂, pyrazinoxy or a solubilizing group. Suitable examples of R³¹ include 3-methoxy-4-((4-methylpiperazin-1-yl)methyl)phenyl, 3-methoxy-4-morpholinomethylphenyl, 3-methoxy-4-diaminomethylphenyl, 3-methoxy-4-((pyrrolidin-1-yl)methyl)phenyl, 3,4-dimethoxyphenyl, 3,5-dimethoxyphenyl, 2,3,4-trimethoxyphenyl, 3,4,5-trimethoxyphenyl, 2-dimethylaminophenyl, 3-dimethylaminophenyl, 4-dimethylaminophenyl, or 3,5-dimethylphenyl.

In certain embodiments, R¹⁹ is selected from

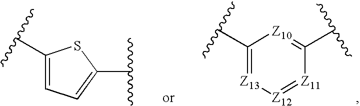

wherein one of Z₁₀, Z₁₁, Z₁₂, and Z₁₃ is N and the others are independently selected from CR²⁰ or CR₁′;

R²¹ is selected from —NH—, —NH—C(O)—, —NH—C(O)—NH, —NH—C(S)—NH— or —NH—S(O)₂—; and

R³¹ is selected from an optionally substituted phenyl, an optionally substituted naphthyl, or an optionally substituted heteroaryl.

In certain such embodiments,
a) when R²¹ is —NH—S(O)₂—, either
   i) Z₁₀ is N; or
   ii) Z₁₁ is N and R³¹ is halophenyl or 2-methoxy-5-methylphenyl;
b) when R¹⁹ is

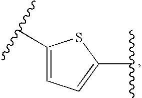

R³¹ is not 4-dimethylaminophenyl, 2,3,4-trimethoxyphenyl, or 3,5 dimethoxyphenyl; and/or
c) when R²¹ is —NH—C(O)—NH— and Z₁₀ is N, R³¹ is not 4-dimethylaminophenyl.

In certain such embodiments, R³¹ is selected from optionally substituted phenyl, benzothiazolyl, or benzoxazolyl.

In yet another embodiment; the invention provides [symbol] -modulating compounds of Structural Formula (VIII):

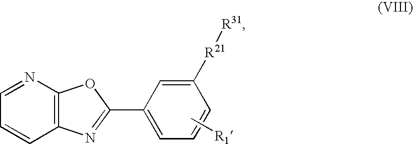

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

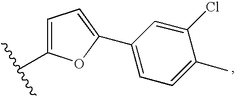

1-methoxynaphthyl, 2-methoxynaphthyl, or unsubstituted 2-thienyl;

when R₁′ is methyl, and R²¹ is —NH—C(O)—CH═CH—, R³¹ is not

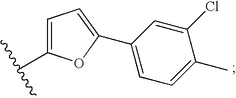

when R₁′ is methyl, and R²¹ is —NH—C(O)—CH—O—, R³¹ is not unsubstituted naphthyl, 2-methoxy, 4-nitrophenyl, 4-chloro-2-methylphenyl, or 4-t-butylphenyl; and

when R²¹ is —NH—C(O)—, R³¹ is not optionally substituted phenyl.

In certain embodiments, R²¹ is —NH—C(O)—; and R³¹ is phenyl optionally substituted with 1 to 3 substituents independently selected from —OCH₃, —CH₃, —N(CH₃)₂, or a solubilizing group.

In certain such embodiments, R²¹ is —NH—C(O)— and R³¹ is selected from unsubstituted phenyl, 2-methoxyphenyl, 3-methoxyphenyl, 2,3,4-trimethoxyphenyl, 3,4,5-trimethoxyphenyl, 2,4-dimethoxyphenyl, 3,5-dimethoxyphenyl, 2-methyl-3-methoxyphenyl, 2-morpholinophenyl, 2-methoxy-4-methylphenyl, 2-dimethylaminophenyl, 4-dimethylaminophenyl, or

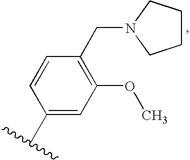

particularly phenyl; 2-methoxyphenyl; 3-methoxyphenyl; 2,3,4-trimethoxyphenyl; 3,4,5-trimethoxyphenyl; 2,4-dimethoxyphenyl; 3,5-dimethoxyphenyl; 2-methyl-3-methoxyphenyl; 2-morpholinophenyl; 2-methoxy-4-methylphenyl; 2-dimethylaminophenyl; or 4-dimethylaminophenyl.

In a further embodiment, the invention provides [symbol] -modulating compounds of Structural Formula (IX):

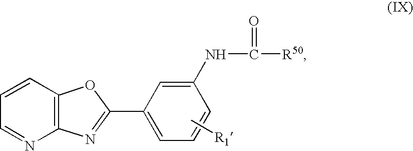

or a salt thereof, wherein:

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one aspect, the invention provides [symbol] modulating compounds of Structural Formula (X):

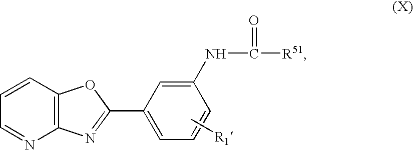

or a salt thereof, wherein:

R₁′ is selected from II or optionally substituted C₁-C₃ straight or branched alkyl; and

R⁵¹ is selected from an optionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl-benzofuranyl, unsubstituted furanyl, phenyl-, bromo-, or nitro-furyl, chlorophenyl-isoxazolyl, oxobenzopyranyl, unsubstituted naphthyl, methoxy-, methyl-, or halo-naphthyl, unsubstituted thienyl, unsubstituted pyridinyl, or chloropyridinyl.

In certain embodiments, R⁵¹ is selected from pyrazolyl, thiazolyl, oxazolyl, pyrimidinyl, furyl, thienyl, pyridyl, isoxazolyl, indolyl, benzopyrazolyl, benzothiazolyl, benzoxazolyl, quinoxalinyl, benzofuranyl, benzothien quinolinyl, benzoisoxazolyl, benzotriazinyl, triazinyl, naphthyl, or

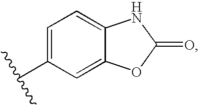

and wherein R⁵¹ is optionally substituted. In certain such embodiments, R⁵¹ is selected from pyrazolyl, thiazolyl, oxazolyl, pyrimidinyl, indolyl, pyrazinyl, triazinyl, or

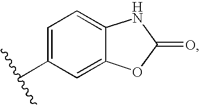

and R⁵¹ is optionally substituted.

In another aspect, the invention provides [symbol] modulating compounds of Structural Formula (XI):

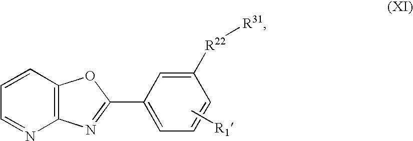

a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected froman optionally substituted monocyclic or bicyclic aryl, or an optionally substituted monocyclic or bicyclic heteroaryl, with the provisos that:

when R²² is —NH—C(O)—CH—CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or unsubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In certain embodiments, R²² is selected from —C(O)—NH—, —NH—, or —C(O)—NH—CH₃.

In certain embodiments, such as when R²² is selected from —C(O)—NH—, —NH—, or —C(O)—NH—CH₃, R³¹ is selected from optionally substituted phenyl, benzothiazolyl, quinoxalinyl, or benzoxazolyl.

In yet another aspect, the invention provides [symbol] -modulating compounds of Structural Formula (XII):

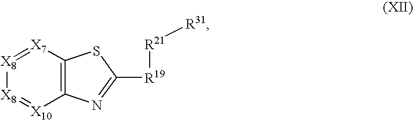

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

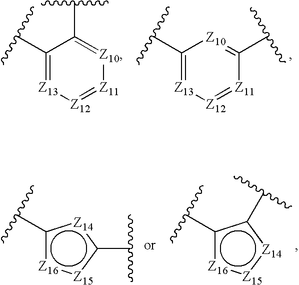

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that when R¹⁹ is

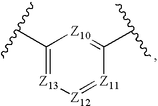

Z₁₀, Z₁₁, Z₁₂ and Z₁₃ are each CH, and R²¹ is —NHIC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

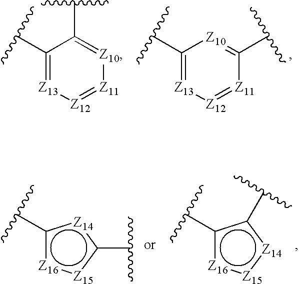

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is R¹⁹ is

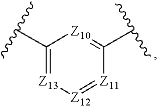

and each of Z₁₀, Z₁₁, Z₁₂ and Z₁₃ is independently selected from CR₂, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In certain embodiments, R²¹ is —NH—C(O)— and R¹⁹ is selected from:

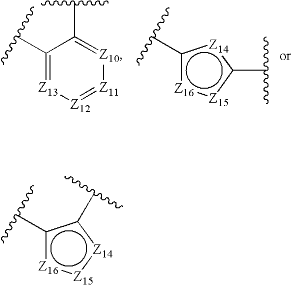

In certain embodiments, R¹⁹ is selected from optionally substituted phenyl, optionally substituted pyridyl, optionally substituted thienyl or optionally substituted furyl.

In certain embodiments, R¹⁹ is

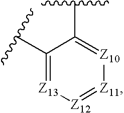

wherein each of Z₁₀, Z₁₁, Z₁₂ and Z₁₃ is independently selected from CR²⁰ or CR₁′; and

R²¹ is selected from —NH—C(O)—, —NH—C(O)—CH(CH₃)—O—, —NH—C(O)—CH₂—O—, or —NH—S(O)₂—CH₂—CH₂—; and

R³¹ is selected from an optionally substituted aryl, or an optionally substituted heteroaryl.

In certain such embodiments, R³¹ is optionally substituted with 1 to 3 substituents independently selected from —OCH₃, —CH₃, —N(CH₃)₂, phenyl, phenoxy, 3,4-dioxymethylene, fluoro, or another solubilizing group. Suitable examples of R³¹ include unsubstituted quinolinyl, 2,4-dimethoxyphenyl, 3,4-dimethoxyphenyl, 3,5-dimethoxyphenyl, 3,4,5-trimethoxyphenyl, 2,3,4-trimethoxyphenyl, 2-dimethylaminophenyl, 3-dimethylaminophenyl, 4-dimethylaminophenyl, 3,5-dimethylphenyl, 3,5-difluorophenyl, 3-trifluoromethoxyphenyl, unsubstituted quinoxalinyl, unsubstituted benzopyrimidinyl,

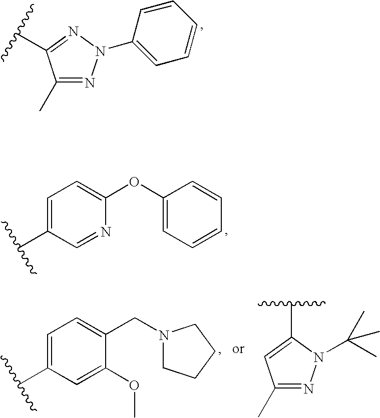

In certain such embodiments, R³¹ is not phenyl-substituted furyl.

In certain embodiments, R¹⁹ is selected from

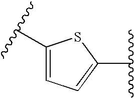

or

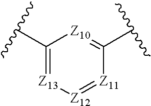

each of Z₁₀, Z₁₁, Z₁₂ and Z₁₃ is independently selected from CR²⁰, or CR₁′;

R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)₂—; and

R³¹ is selected from an optionally substituted phenyl, an optionally substituted naphthyl, or an optionally substituted heteroaryl.

In certain such embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

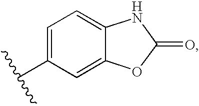

and R³¹ is optionally substituted (e.g., optionally substituted with up to three substituents independently selected from —OCH₃, —CH₃, —N(CH₃)₂, —O-phenyl, or another solubilizing group). Suitable examples of R³¹ include unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl.

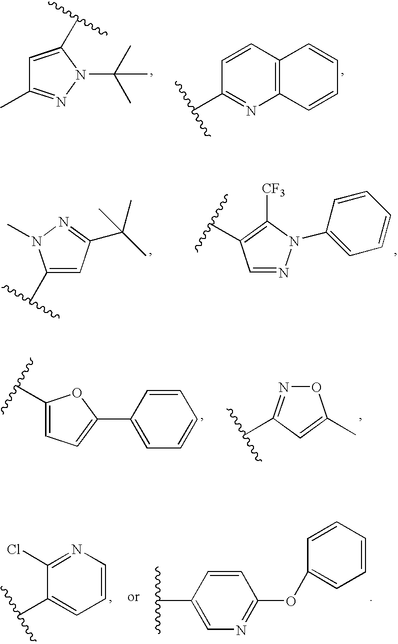

In certain embodiments, one or more of the following conditions applies:

when X₈ is N, R²¹ is —NH—C(S)—NH—, and R¹⁹ is phenyl, R³¹ is not 2-methoxy-5-nitrophenyl, 2-S-methylphenyl or 2-acetylphenyl;

when X₈ is N, R²¹ is —NH—S(O)₃—, and R¹⁹ is phenyl, R³¹ is not thiadiazole-substituted thienyl or 4-methylsulfonylphenyl;

when X₈ is N, R²¹ is —NH—CO—, and R¹⁹ is phenyl, R³¹ is not 2,4-difluorophenyl, pyridyl-substituted thienyl, 3,4-dichlorophenyl, 4-t-butylphenyl, or 3-benzyloxyphenyl;

when X₉ is N, R²¹ is —NH—C(O)— and R¹⁹ is

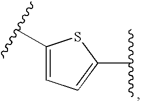

R³¹ is not 2,3,4-trimethoxyphenyl or 3,5-dimethoxyphenyl; and

when X₉ is N, R²¹ is —NH—C(O)— and R¹⁹ is phenyl, R³¹ is not 3,5-dimethoxyphenyl.

In a further embodiment, the invention provides compounds of Structural Formula (XIII):

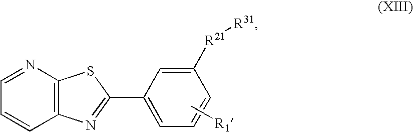

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′=CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethylphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂—CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In certain embodiments, R₁′ is selected from H or optionally substituted C₁-C₃ straight or branched alkyl;

R²¹ is selected from —NR₁′—C(O)—; —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from a monocyclic or bicyclic aryl or a monocyclic or bicyclic heteroaryl, and comprises a solubilizing group substituent.

In certain embodiments, R³¹ is selected from phenyl, naphthyl, pyrazolyl, furyl, thienyl, pyridyl, isoxazolyl, benzopyrazolyl, benzofuryl, benzothienyl, quinolinyl, benzoisoxazolyl, or

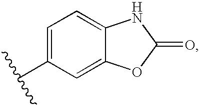

and R³¹ is optionally substituted.

In certain embodiments, R²¹ is selected from —NH—C(O)—, NH—C(O)—CH₂—CH(CH₃)—O, —NH—C(O)—NH—, —NH—C(S)—NH—, —NH—C(S)—NH—CH₂—, or —NH—S(O)—; and

R³¹ is selected from an optionally substituted phenyl, an optionally substituted naphthyl, or an optionally substituted heteroaryl.

In certain such embodiments, particularly when R²¹ is —NH—C(O)—, R³¹ is selected from R³¹ is selected from unsubstituted phenyl, 3-methoxyphenyl, 4-methoxyphenyl, 2,3 dimethoxyphenyl, 2,4-dimethoxyphenyl, 2,5-bis(trifluoromethyl)phenyl, 3,4-dimethoxyphenyl, 3,5-dimethoxyphenyl, 3,4,5-trimethoxyphenyl, 2,3,4-trimethoxyphenyl, 2-methoxy-4-methylphenyl, 2-phenoxyphenyl, 3-dimethylaminophenyl, 4-dimethylaminophenyl, unsubstituted 2-furanyl, unsubstituted 2-thienyl,

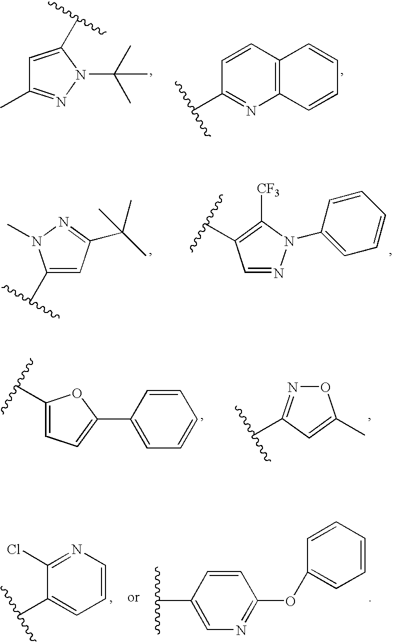

In one aspect, the invention provides [symbol] -modulating compounds of Structural Formula (XIV):

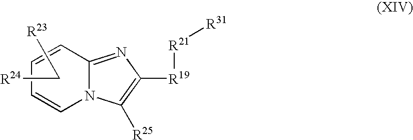

or a salt thereof, wherein:

each of R²³ and R²⁴ is independently selected from H, —CH₃ or a solubilizing group;

R²⁵ is selected from H or a solubilizing group; and

R¹⁹ is selected from:

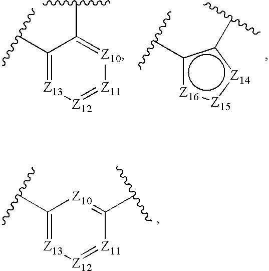

or

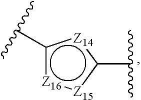

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′, —C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′—; and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

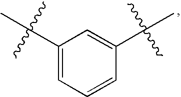

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In certain embodiments, each of R²³ and R²⁴ is independently selected from H, —CH₃ or a solubilizing group;

R²⁵ is selected from H, or a solubilizing group; and

R¹⁹ is selected from:

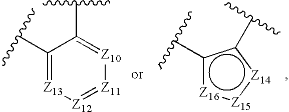

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′, —O—, —NR₁′—C(O)—CR₁′R₁′, —CR₁′R₁′—O—, —NR₁′—S(O)₂—CR₁′R₁′—, —NR₁′—S(O)₂—CR₁′R₁′—CR₁′R₁′—NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, R³¹ is not 2,4-dimethoxyphenyl.

Typically, R²⁵ is selected from H, —CH₂—N(CH₃)₂, or

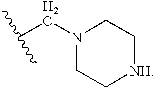

Typically, R²³ and R²⁴ are H.

Typically, R¹⁹ is selected from phenyl, pyridyl, thienyl or furyl, particularly optionally substituted phenyl. Preferably, a phenyl is optionally substituted with:

a) up to three —O—CH₃ groups; or

b) one —N(CH₃)₂ group.

In certain embodiments, each of R²³ and R²⁴ is independently selected from H, —CH₃ or a solubilizing group;

R²⁵ is selected from H, or a solubilizing group; and

R¹⁹ is selected from

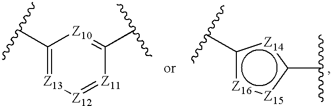

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ is N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is Selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′, —NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,

wherein when R¹⁹ is phenyl, at least one of R²³, R²⁴, or R²⁵ is a solubilizing group and wherein said compound is not 2-chloro-N-[3-[3-(cyclohexylamino)imidazo[1,2-a]pyridin-2-yl]phenyl]-4-nitrobenzamide.

Typically, R²⁵ is selected from —H, —CH₂—N(CH₃)₂, or

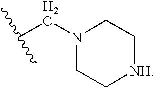

Typically, R²³¹ and R²⁴ are H.

Typically, R¹⁹ is selected from phenyl, pyridyl, thienyl or furyl, particularly optionally substituted phenyl. Preferably, a phenyl is optionally substituted with:

b) up to three —O—CH₃ groups; or

b) one —N(CH₃)₂ group.

In another aspect, the invention provides [symbol] -modulating compounds of Structural Formula (XV):

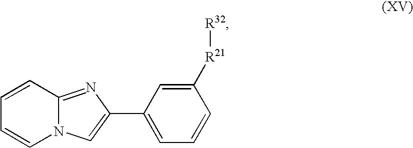

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, NR₁′—S(O)₂CR₁′R₁′—CR₁′R₁′—, —NR₁′—C(O)—CR₁′R₁′—CR₁′R₁′—, —NR₁′—C(S)—NR₁′—CR₁′R₁′CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

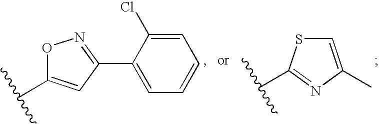

and

when R²¹ is —NR₁′—S(O)₂—, R³² is not unsubstituted 2-thienyl or unsubstituted naphthyl.

In yet another aspect, the invention provides [symbol] -modulating compounds of Structural Formula (XVI):

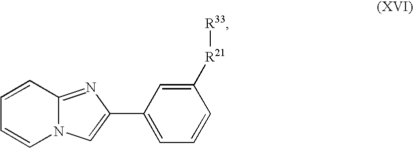

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′, —NR₁′—C(O)—CR₁′R₁′—NR₁′—, —NR₁′C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—, —C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, —NR₁′—C(═N—CN)—NR₁′—, —NR₁′—C(O)—CR₁′R₁′—CR₁′R₁′—, —NR₁′—C(S)—NR₁′—CR₁′R₁′—CR₁′R₁′—, —NR₁′—C(O)—O— or —NR₁′—C(O)—CR₁′R₁′— (particularly —NH—C(O)—); and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′— or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In certain embodiments, R²¹ is selected from —NR²²—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—CR₁′—, —NR₁′—S(O)₂—NR₁′—, —NR₁′—C(O)—NR₁′—S(O)₂—, —NR₁′—CR₁′R₁′—, —C(O)—NR₁′—, —CR₁′R₁′—C(O)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′═CR₁′—CR₁′R₁′—, —NR₁′—C(═N—CN)—NR₁′—, or —NR₁′—C(O)—CR₁′R₁′—; and

each R₁′ is independently selected from H or optionally substituted C₁-C₃ straight or branched alkyl;

R²² is an optionally substituted C₁-C₃ straight or branched alkyl; and

R³³ is phenyl comprising a solubilizing group substituent, wherein: when R²¹ is —NH—S(O)₂ said phenyl comprises an additional substituent.

In certain embodiments, R²¹ is selected from —NR²²—C(O)—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′R₁′—CR₁′R₁′—, —NR₁′—S(O)₂—CR₁′R₁′—, —NR₁′—S(O)₂—CR₁′R₁′—CR₁′R₁′—, —NR₁′—C(O)—CR₁′═CR₁—CR₁′R₁′—, or —NR₁′—C(═N—CN)—NR₁′—,

each R₁′ is independently selected from H or optionally substituted C₁-C₃ straight or branched alkyl; and

R²² is an optionally substituted C₁-C₃ straight or branched alkyl.

In certain embodiments, R³³ is optionally substituted on up to three carbon atoms with a substituent independently selected from —O—CH₃, —CH₃, —N(CH₃)₂, —S(CH₃), or CN; or substituted on adjacent carbon atoms with

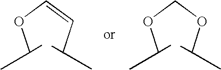

bridging said adjacent carbon atoms.

In a further embodiment, the invention provides [symbol] modulating compounds of Structural Formula (XVII):

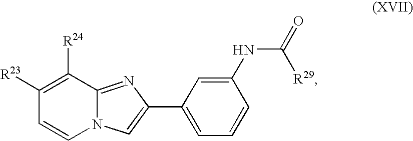

or a salt thereof, wherein:

each of R²³ and R²⁴ is independently selected from H or —CH₃, wherein at least one of R³ and R²⁴ is H; and

R²⁹ is phenyl substituted with:

a) two —O—CH₃ groups;

b) three —O—CH₃ groups located at the 2, 3 and 4 positions; or

c) one —N(CH₃)₂ group; and;

d) when R²³ is CH₃, one —O—CH₃ group at the 2 or 3 position,

wherein R²⁹ is optionally additionally substituted with a solubilizing group.

In certain embodiments, R²⁹ is phenyl substituted with:

a) three —O—CH₃ groups located at the 2, 3 and 4 positions; or

b) one —N(CH₃)₂ group.

In one aspect, the invention provides [symbol] -modulating compounds of Structural Formula (XVIII):

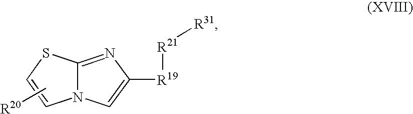

or a salt thereof, wherein

R¹⁹ is selected from:

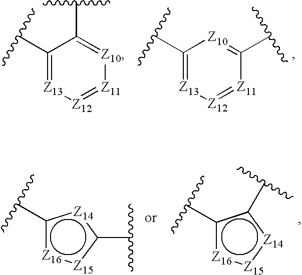

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

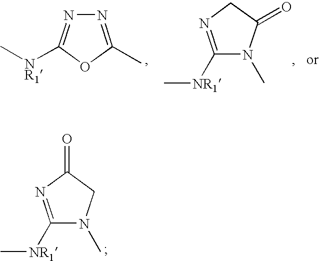

wherein each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, with the proviso that when R¹⁹ is

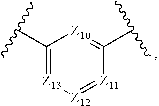

Z₁₀, Z₁₁, Z₁₂ and Z₁₃ are each CH, R²⁰ is H, and R²¹ is —NHC(O)—, R³¹ is not an optionally substituted phenyl.

In certain embodiments, R¹⁹ is selected from:

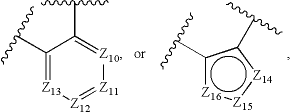

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, O or S;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

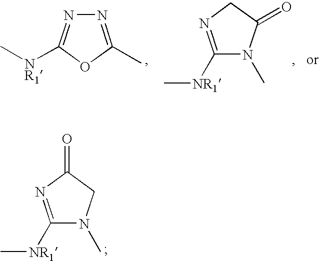

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl.

In certain such embodiments, compounds of Structural Formula (XVIII) have the formula:

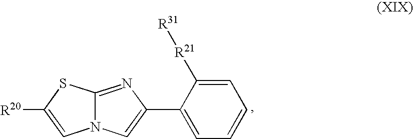

or a salt thereof, wherein

R²⁰ is selected from H or a solubilizing group;

R²¹ is selected from —NH—C(O)—, or —NH—C(O)—CH₂—; and

R³¹ is selected from an optionally substituted monocyclic or bicyclic aryl, or an optionally substituted monocyclic or bicyclic heteroaryl.

Typically, R¹⁹ in compounds of Structural Formula (XVIII) is selected from phenyl, pyridyl, thienyl or furyl, particularly optionally substituted phenyl.

Typically R²⁰ is selected from H, —CH₂—(CH₃)₂,

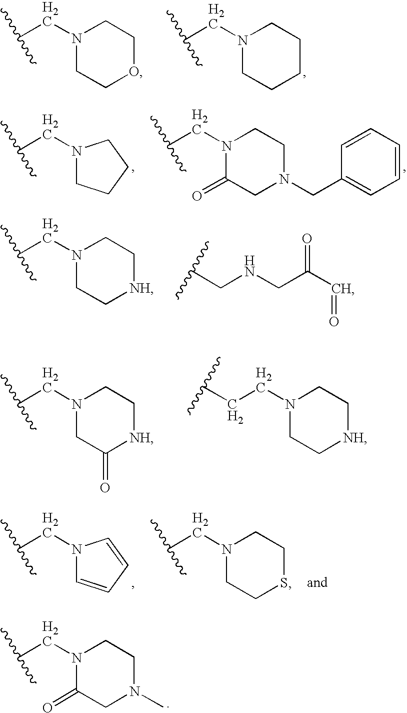

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In certain such embodiments when R²¹ is —NR₁′—C(O)—, R³¹ is not 4-cyanophenyl or

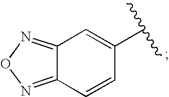

and/or when R²¹ is —NR₁′—S(O)₂—, R³¹ is not 4-methoxyphenyl or 4-t-butylphenyl.

In certain such embodiments, when R¹⁹ is

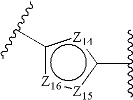

or

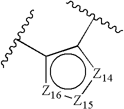

and R²¹ is —NR₁′—C(O)—, R³¹ is not 4-cyanophenyl or

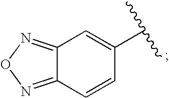

and/or when R¹⁹ is

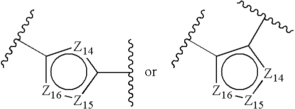

and R²¹ is —NR₁′—S(O)₂—, R³¹ is not 4-methoxyphenyl or 4-t-butylphenyl.

In another aspect, the invention provides [symbol] -modulating compounds of Structural Formula (XX):

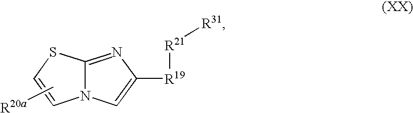

or a salt thereof, wherein

R¹⁹ is selected from:

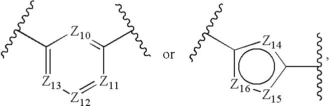

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from 1-1 or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

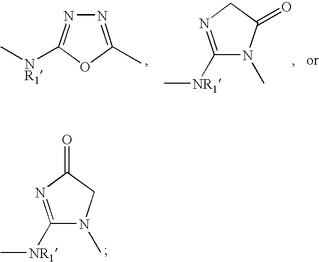

wherein
each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl, wherein when R¹⁹ is

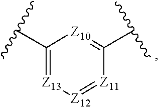

and Z₁₀, Z₁₁, Z₁₂ and Z₁₃ are each CH, R^{20a} is a solubilizing group.

Typically, R¹⁹ in compounds of Structural Formula (XX) is selected from phenyl, pyridyl, thienyl or furyl, particularly optionally substituted phenyl.

Typically, R^{20a} is selected from H, —CH₂—N(CH₃)₂,

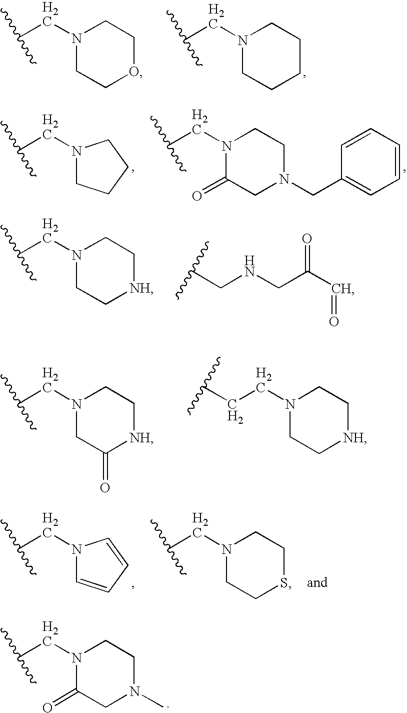

Typically, R³¹ is selected from phenyl, pyrazolyl, furyl, pyridyl, pyrimidinyl, thienyl, naphthyl, benzopyrazolyl, benzofuryl, quinolinyl, quinoxalinyl, or benzothienyl and wherein R³¹ is optionally substituted.

Typically, R²¹ is selected from —NH—C(O)— or —NH—C(O)—CH₂—.

In yet another aspect, the invention provides [symbol] modulating compounds of Structural Formula (XXI):

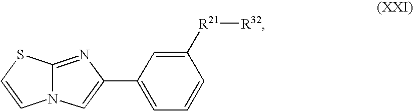

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

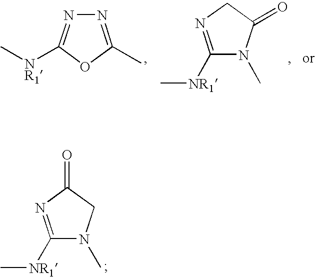

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R²¹ is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In certain embodiments, R³² is selected from pyrrolyl, pyrazolyl, pyrazinyl, furyl, pyridyl, pyrimidinyl, or thienyl, and R³² is optionally substituted and is optionally benzofused.

In certain embodiments, R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—; —NR₁′—C(O)—CR₁′R₁′—CR₁′R₁′—, —NR₁′—C(S)—NR₁′—CR₁′R₁′—CR₁′R₁′—, —NR₁′—C(O)—O—,

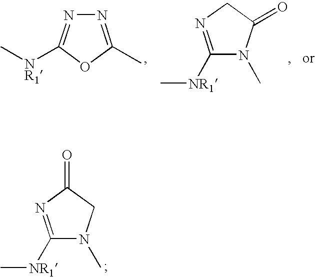

wherein
each R₁′ is independently selected from H or optionally substituted C₁-C₃ straight or branched alkyl; and

R³² is selected from benzofuryl, methylfuryl, benzothienyl, pyridyl, pyrazinyl, pyrimidinyl, pyrazolyl, wherein said methylfuryl, pyridyl, pyrazinyl, pyrimidinyl or pyrazolyl is optionally benzofused and wherein R³² is optionally substituted or further substituted.

In a further aspect, the invention provides [symbol] -modulating compounds of Structural Formula (XXII):

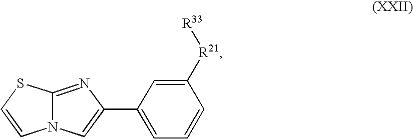

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁—, —CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—, —NR₁′—C(O)—CR₁′R₁′—CR₁′R₁′—O—, —NR₁′—C(O)—CR₁′R₁′—O—, —NR₁′—S(O)₂—CR₁′R₁′—, —NR₁′—S(O)₂—CR₁′R₁′—CR₁′R₁′—,

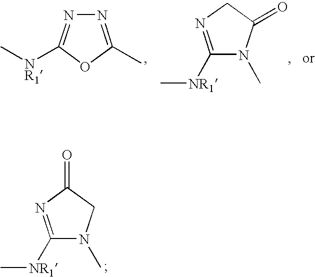

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

Preferably, R²¹ is selected from —NH—C(O)— or —NH—C(O)—CH₂—.

In one aspect, the invention provides [symbol] -modulating compounds of Structural Formula (XXII):

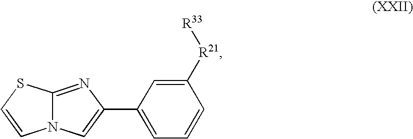

or a salt thereof wherein:

R²¹ is selected from —NH—C(O)—, or —NH—C(O)—CH₂—; and

R³³ is phenyl substituted with

e) one —N(CH₃)₂ group;

f) one CN group at the 3 position;

g) one —S(CH₃) group; or

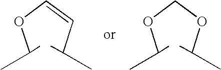

bridging the 3 and 4 positions.

In another aspect, the invention provides [symbol] -modulating compounds of Structural Formula (XXIII):

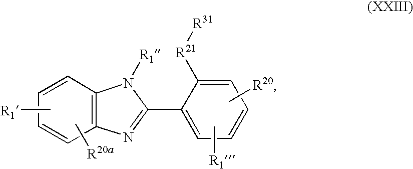

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—; —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-butoxyphenyl

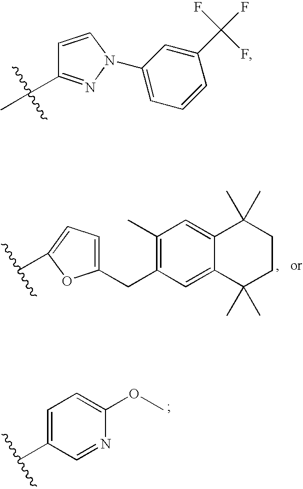

when R²¹ is —NH—C(O)— and each of R²⁰, R^{20a}, R₁′, R₁″ and R₁′″ is hydrogen, R₃₁ is not

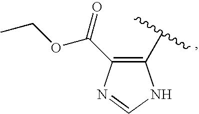

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;
when R²¹ is —NH—C(O)—, R₁″ is methyl; and each of R²⁰, R^{20a}, R₁′ and R₁′″ is hydrogen, R³¹ is not 2-methylaminophenyl,

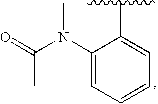

or

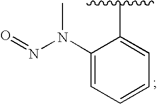

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;
when R²¹ is —NH—S(O)₂—, R₁″ is hydrogen or methyl, and each of R²⁰, R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

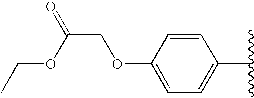

or

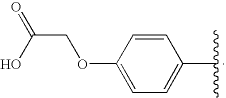

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In a particular aspect, the invention provides [symbol] -modulating compounds of Structural Formula (XXIII):

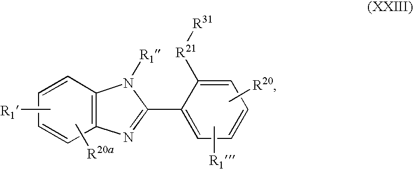

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—; —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl,
wherein:
i) at least one R²⁰ is a solubilizing group or at least one R₁′″ is an optionally substituted C₁-C₃ straight or branched alkyl or both; or
ii) R^{20a} is a solubilizing group other than CH₂—N(CH₂CH₃)₂.

In certain embodiments, R²¹ is selected from —NH—C(O)—, or —NH—C(O)—NR₁′—.

In certain embodiments, R³¹ is selected from optionally substituted phenyl, quinoxalinyl or quinolinyl. For example, R³¹ is optionally substituted with up to 3 substituents independently selected from —OCH₃, —N(CH₃)₂, or a solubilizing group. Suitable examples of R³¹ include 4-dimethylaminophenyl, 3,4-dimethoxyphenyl, 3,5-dimethoxyphenyl, 3,4,5-trimethoxyphenyl, 3-methoxy-4-((piperazin-1-yl)methyl)phenyl, 3-methoxy-4-((morpholino)methyl)phenyl, 3-methoxy-4-((pyrrolidin-1-yl)methyl)phenyl, unsubstituted phenyl, unsubstituted quinoxalinyl, and unsubstituted quinolinyl.

In yet another aspect, the invention provides [symbol] -modulating compounds of Structural Formula (XXIV):

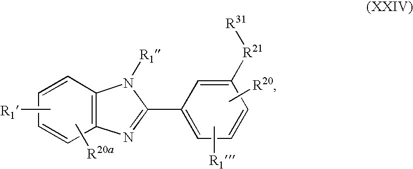

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—CH₂—, R³¹ is not 2-methylphenyl, or 3,4-dimethoxyphenyl;
when R²¹ is —NH—C(O)—CH═CH—, R³¹ is not 2-chlorophenyl;
when R²¹ is —NH—C(O)—NH—, R³¹ is not unsubstituted benzimidazolyl;
when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;
when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;
when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1-dimethylethyl)phenyl, or unsubstituted phenyl;
when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

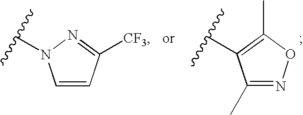

when R²¹ is —NH—C(O)—CH₂—, R₁′ is methyl, and each of R²⁰, R^{20a}, R₁″, and R₁′″ is hydrogen, R³¹ is not unsubstituted phenyl;
when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;
when R²¹ is —NH—C(O)—CH(CH₂CH₃)—, and each of R²⁰, R^{20a}, R₁′, R₁″, and R₁′″ is hydrogen, R³¹ is not unsubstituted phenyl;
when R²¹ is —NH—C(O)—CH(CH₃)—O—, R₁′″ is methyl or hydrogen, and each of R²⁰, R^{20a}, R₁′, and R₁″ is hydrogen, R³¹ is not 2,4-dichlorophenyl.

In a particular aspect, the invention provides [symbol] -modulating compounds of Structural Formula (XXIV):

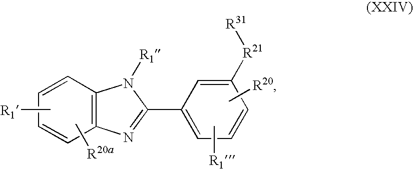

or a salt thereof, wherein:
each R²⁰ and R^{20a} is independently selected from H or a solubilizing group and at least one of R²⁰ and R^{20a} is a solubilizing group;
each R₁′, R₁″ and R₁′″ is independently selected from H or optionally substituted C₁-C₃ straight or branched alkyl;
R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and
R³¹ is selected from an optionally substituted monocyclic or bicyclic aryl, or an optionally substituted monocyclic or bicyclic heteroaryl.

In certain embodiments, when R²¹ is —NH—C(O)—CH₂—, R³¹ is not 2-methylphenyl; or 3,4-dimethoxyphenyl; when R²¹ is —NH—C(O)—CH═CH—, R³¹ is not 2-chlorophenyl; and/or when R²¹ is —NH—C(O)—NH—, R³¹ is not unsubstituted benzimidazolyl.

In a further aspect, the invention provides [symbol] -modulating compounds of Structural Formula (XXV):

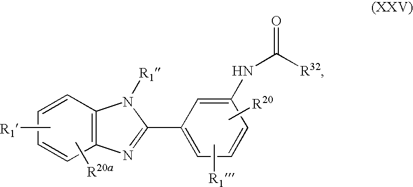

or a salt thereof, wherein:
each R²⁰ and R^{20a} is independently selected from H or a solubilizing group, wherein at least one of R²⁰ and R^{20a} is a solubilizing group;
each R₁′, R₁″ and R₁′″ is independently selected from H or optionally substituted C₁-C₃ straight or branched alkyl; and
R³² is an optionally substituted phenyl.

In certain embodiments, R³² is selected from 3,4-dimethoxyphenyl, 2,6-dimethoxyphenyl, or 2,4-dimethoxyphenyl; wherein R³² is further optionally substituted with a solubilizing group.

In certain embodiments, R³² is not unsubstituted thienyl; unsubstituted phenyl; 2-methylphenyl; 4-fluorophenyl; 4-methoxyphenyl; 4-methylphenyl; 3,4-dioxyethylenephenyl; 3-acetylamino-4-methylphenyl; 3-[(6-amino-1-oxohexyl)amino]-4-methylphenyl; 3-amino-4-methylphenyl; 3,5-dimethoxyphenyl; 3-halo-4-methoxyphenyl; 3-nitro-4-methylphenyl; or 4-propoxyphenyl.

In one aspect, the invention provides [symbol] -modulating compounds of Structural Formula (XXVI):

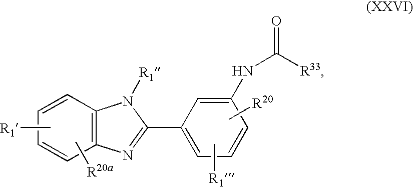

or a salt thereof, wherein:
each R²⁰ and R^{20a} is independently selected from H or a solubilizing group;
each R₁′, R₁″ and R₁′″ is independently selected from H or optionally substituted C₁-C₃ straight or branched alkyl; and
R³³ is selected from an optionally substituted heteroaryl or an optionally substituted bicyclic aryl, with the provisos that:
when each of R₁′ and R₁′″ is hydrogen or methyl and each of R₁″, R₂₀ and R₂₀ₐ is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

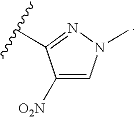

In a particular aspect, the invention provides [symbol] -modulating compounds of Structural Formula (XXVI):

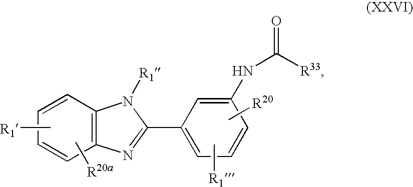

or a salt thereof, wherein:
each R²⁰ and R^{20a} is independently selected from H or a solubilizing group, wherein at least one of R²⁰ or R^{20a} is a solubilizing group;
each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In another aspect, the invention provides [symbol] -modulating compounds of Structural Formula (XXVII):

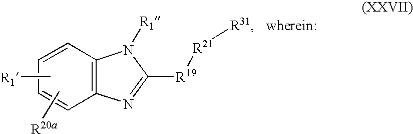

each R²⁰ and R^{20a} is independently selected from H or a solubilizing group;
each R₁′ and R₁″ is independently selected from H or optionally substituted C₁-C₃ straight or branched alkyl;

R¹⁹ is selected from:

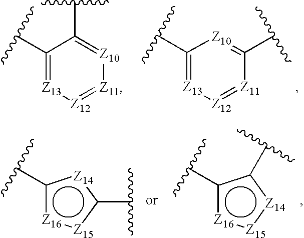

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR 1′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

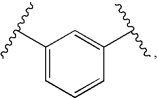

R³¹ is not unsubstituted pyridyl, 2,6-dimethoxyphenyl, 3,4,5-trimethoxyphenyl or unsubstituted furyl.

In a particular aspect, the invention provides [symbol] modulating compounds of Structural Formula (XXVII):

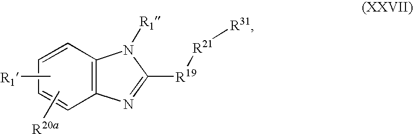

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

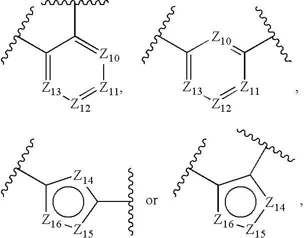

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—, and R¹⁹ is thiazolyl, R³¹ is not optionally substituted phenyl or optionally substituted pyridyl;
when R²¹ is —NH—C(O)—CH₂—, and R¹⁹ is pyrazolyl, R³¹ is not unsubstituted indolyl or unsubstituted phenyl;
when R²¹ is —NH—C(O)—CH₂—, and R¹⁹ is

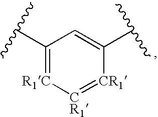

R³¹ is not 2-methylphenyl or 3,4-dimethoxyphenyl;
when R²¹ is —NH—C(O)—CH═CH—, and R¹⁹ is

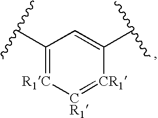

R³¹ is not 2-chlorophenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;
when R²¹ is —NH—C(O)—NH—, and R¹⁹ is

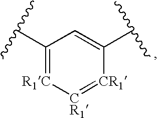

R³¹ is not unsubstituted benzimidazolyl;
when R² is —NH—, and R¹⁹ is pyrazolyl, R³¹ is not unsubstituted pyridyl;
when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;
when R²¹ is —NH— and R¹⁹ is pyridyl, oxadiazolyl or thiadiazolyl, R³¹ is not unsubstituted phenyl, 3-methoxyphenyl or 4-methoxyphenyl;
when R²¹ is —NH—C(O)— and R¹⁹ is thiazolyl or pyrimidin R³¹ is not unsubstituted phenyl;
when R²¹ is —NH—C(O)— and R¹⁹ is

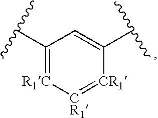

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;
when R²¹ is —NH—C(O)— and R¹⁹ is

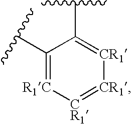

R³¹ is not 3,5-dinitrophenyl, 4-butoxyphenyl,

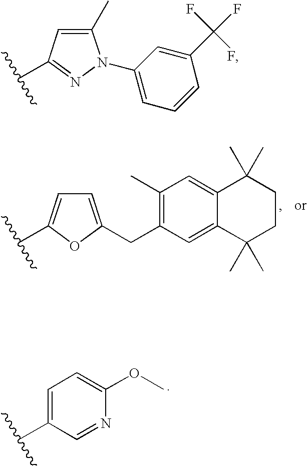

In certain embodiments, R²¹ is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In certain embodiments, when R²¹ is —NH—C(O)—, R¹⁹ is not

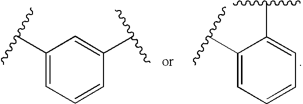

In certain embodiments, when R²¹ is —NH—C(O)—, R¹⁹ is not optionally substituted pyrazolyl, thiazolyl, thienyl, pyrrolyl or pyrimidinyl; when R²¹ is —NH—C(O)—CH₂— or —NH—C(O)—NH—, R¹⁹ is not pyrazolyl; and/or when R²¹ is —NH—, R¹⁹ is not optionally substituted pyridyl, thiazolyl, pyrazolyl, thiadiazolyl, or oxadiazolyl.

In a more particular aspect, the invention provides [symbol] -modulating compounds of Structural Formula (XXVII):

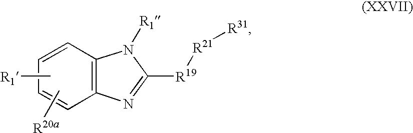

or a salt thereof, wherein:
each R²⁰ and R^{20a} is independently selected from H or a solubilizing group;
each R₁′ and R₁″ is independently selected from H or optionally substituted C₁-C₃ straight or branched alkyl;
R¹⁹ is selected from:

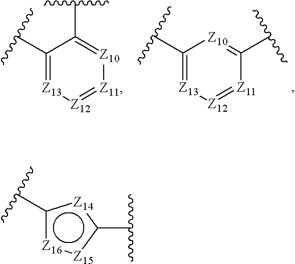

or

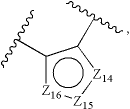

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR₂, or CR₁′, wherein:
one to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl, with the provisos that:
when R²¹ is —NH—C(O)—, R¹⁹ is not pyrazolyl;
when R²¹ is —NH—C(O)—CH₂—, and R¹⁹ is pyrazolyl, R³¹ is not unsubstituted indolyl or unsubstituted phenyl;
when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;
when R²⁰ is a solubilizing group, R¹⁹ is 1-methylpyrrolyl and R²¹ is —NH—C(O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;
when R^{20a} is a solubilizing group, R¹⁹ is thienyl and R²¹ is —NH—C(O)—, R³¹ is not unsubstituted phenyl;
when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group; and
when R²¹ is —NH—C(O)— and R¹⁹ is thiazolyl or pyrimidinyl, R³¹ is not unsubstituted phenyl.

In certain embodiments, R² is selected from —NH—C(O)— or —NH—C(O)—NR₁′—, preferably —NH—C(O)—.

In certain embodiments, R³¹ is selected from optionally substituted phenyl, quinoxalinyl or quinolinyl; preferably optionally substituted phenyl. For example, R³¹ is optionally substituted with up to 3 substituents independently selected from —OCH₃, —N(CH₃)₂, or a solubilizing group. Suitable examples of R³¹ include 4-dimethylaminophenyl; 3,4-dimethoxyphenyl; 3,5-dimethoxyphenyl; 3,4,5-trimethoxyphenyl; 3-methoxy-4-((piperazin-1-yl)methyl)phenyl; 3-methoxy-4-((morpholino)methyl)phenyl; 3-methoxy-4-((pyrrolidin-1-yl)methyl)phenyl; unsubstituted phenyl; unsubstituted quinoxalinyl; and unsubstituted quinolinyl. Preferred examples of R³¹ include 3,4-dimethoxyphenyl; 2,6-dimethoxyphenyl; or 2,4-dimethoxyphenyl; wherein R³¹ is further optionally substituted with a solubilizing group.

In preferred embodiments, R²¹ is —NH—C(O)— and R³¹ is selected from 3-methoxyphenyl; 3,4-dimethoxyphenyl; 3,4,5-trimethoxyphenyl; or 4-dimethylaminophenyl.

In yet another aspect, the invention provides compounds of Structural Formula (XXVIII):

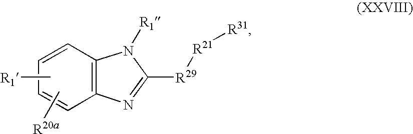

or a salt thereof, wherein:
each R²⁰ and R^{20a} is independently selected from H or a solubilizing group;
each R₁′ and R₁″ is independently selected from H or optionally substituted C₁-C₃ straight or branched alkyl;
R²⁹ is selected from:

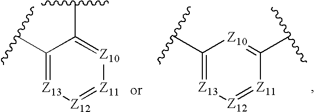

wherein:
each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and
zero to one R²⁰ is a solubilizing group;
zero to one R₁′″ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and
R³¹ is selected from an optionally substituted monocyclic or bicyclic aryl, or an optionally substituted monocyclic or bicyclic heteroaryl.

In certain embodiments, R³¹ is optionally substituted phenyl, such as 3-methoxyphenyl, 3,4-dimethoxyphenyl, 3,4,5-trimethoxyphenyl, or 4-dimethylaminophenyl.

In certain embodiments, R²¹ is —NH—C(O)—.

In preferred embodiments, R²¹ is —NH—C(O)— and R³¹ is an optionally substituted phenyl, such as 3-methoxyphenyl, 3,4-dimethoxyphenyl, 3,4,5-trimethoxyphenyl, or 4-dimethylaminophenyl.

In a further aspect, [symbol] , the invention provides novel [symbol] -modulating compounds of Formula (VI):

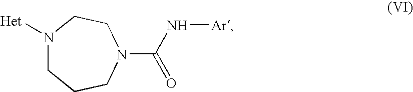

or a salt thereof, wherein:

Het is an optionally substituted heterocyclic aryl group; and

Ar′ is an optionally substituted carbocyclic or heterocyclic aryl group.

In certain embodiments, Het comprises one N heteroatom and 1 to 2 additional heteroatoms independently selected from N, O or S, such as oxazolopyridyl.

In certain embodiments, Ar′ is selected from optionally substituted phenyl, benzothiazolyl, or benzoxazolyl. When Ar′ is substituted phenyl, typically it is substituted with 1 to 3 substituents independently selected from halo, methyl, O-methyl, S-methyl or N(CH₃)₂, morpholino, or 3,4 dioxymethylene.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

In the compounds described above, bivalent groups disclosed as possible values for variables can have either orientation, provided that such orientation results in a stable molecule. Preferably, however, the left hand side of a bivalent group (e.g., —NR₁′—C(O)—) is attached to a bivalent arylene or heteroarylene group (e.g., R¹⁹) and the right hand side of a bivalent group is attached to a monovalent aryl group (e.g., R³¹).
[symbol] modulating compounds of the invention having hydroxyl substituents, unless otherwise indicated, also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

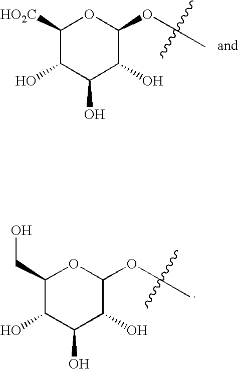

These groups are generally cleavable to —OH by hydrolysis or by metabolic (e.g., enzymatic) cleavage.

In certain embodiments, the compounds of the invention exclude one or more of the species disclosed in Tables 4-6. In certain such embodiments, the compounds of the invention exclude compound 7.
[symbol] -modulating compounds of the invention advantageously modulate the level and/or activity of a protein,

[symbol]

Separately or in addition to the above properties, certain [symbol] -modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A ring (e.g., 5- to 7-membered ring) or cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuryl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}); —NRCONH₂, —NRCCONR^{a}H, —NRCCON(R^{a}R^{b}), —C(═NH)—NH₂, —C(═NH)—NHR^{a}, —C(═NH)—N(R^{a}R^{b}), —C(═NR^{c})—NH₂, —C(═NRC)—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

A hydrogen-bond donating group is a functional group having a partially positively-charged hydrogen atom (e.g., —OH, —NH₂, —SH) or a group (e.g., an ester) that metabolizes into a group capable of donating a hydrogen bond.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R^{a}—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C6-C14) aryl, phenyl, naphthyl, (C7-C20) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)ₙ—C(O)—R^{b}, —(CH₂)ₙ—O—(CH₂)ₙ—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or R^{b} moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula: —(CH₂)ₙ—R¹⁰⁰—N(R¹⁰¹)(R¹⁰¹), wherein:

n is selected from 0, 1 or 2;
R¹⁰⁰ is selected from a bond, —C(O)—, or —O(CH₂)ₙ; and
each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;
c.

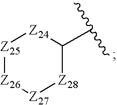

d.

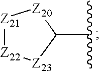

e.

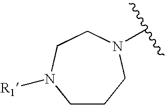

or
f. both R¹⁰¹ moieties are taken together with the nitrogen atom to which they are bound to form a ring of the structure

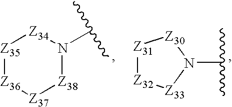

or

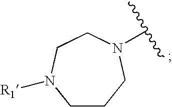

or
g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′;
   wherein:

each Z is independently selected from —O—, —S—, —NR₁′—, or —C(R⁵⁰)(R⁵⁰)—,

wherein:
at least three of Z₂₀, Z₂₁, Z₂₂, and Z₂₃ are —C(R⁵⁰)(R⁵⁰)—;
at least three of Z₂₄, Z₂₅, Z₂₆, Z₂₇, and Z₂₈ are —C(R⁵⁰)(R⁵⁰)—;
at least four of Z₃₀, Z₃₁, Z₃₂, and Z₃₃ are —C(R⁵⁰)(R⁵⁰)—; and
at least four of Z₃₄, Z₃₅, Z₃₆, Z₃₇, and Z₃ s are —C(R⁵⁰)(R⁵⁰)—;

each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;

each R⁵⁰ is independently selected from R₁′, halo, CN, OH, O—(C₁-C₄ straight or branched alkyl), N(R₁′)(R₁′), ═CR₁′, SR₁′, ═NR₁′, ═NOR₁′, or ═O;

any two suitable non-cyclic R⁵⁰ are optionally bound to one another directly or via a C₁ to C₂ alkylene, alkenylene or alkanediylidene bridge to produce a bicyclic fused or spiro ring, and any

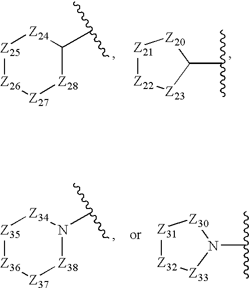

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another preferred embodiment, the solubilizing group is a moiety of the formula: —(CH₂), —O—R¹⁰¹, wherein n and R¹⁰¹ are as defined above.

In another preferred embodiment, the solubilizing group is a moiety of the formula; —(CH₂)ₙ—C(O)—R₁′, wherein n and R₁′ are as defined above.

In a more preferred embodiment, a solubilizing group is selected from —(CH₂)ₙ—R¹⁰², wherein n is 0, 1 or 2; and R¹⁰² is selected from

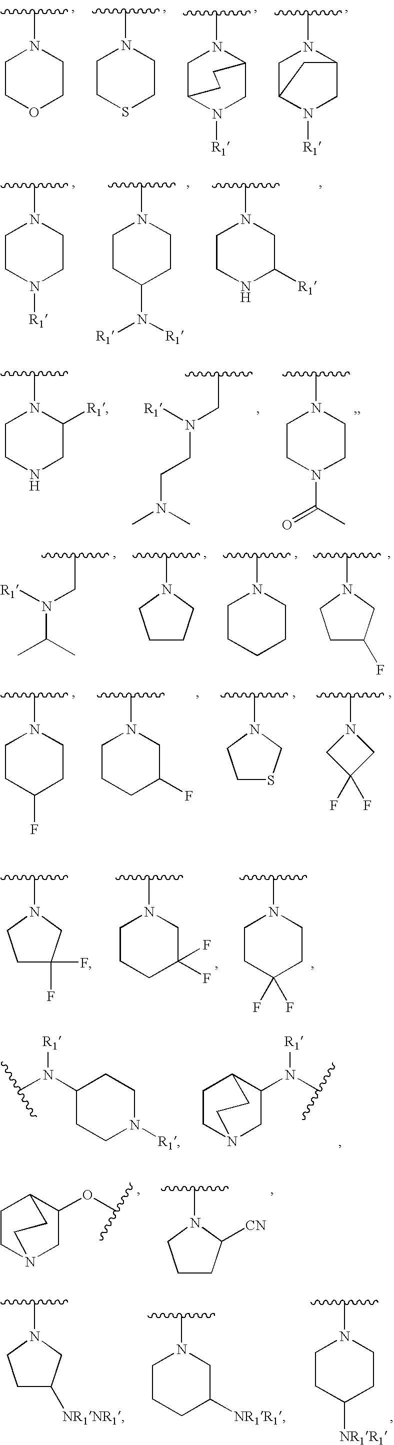

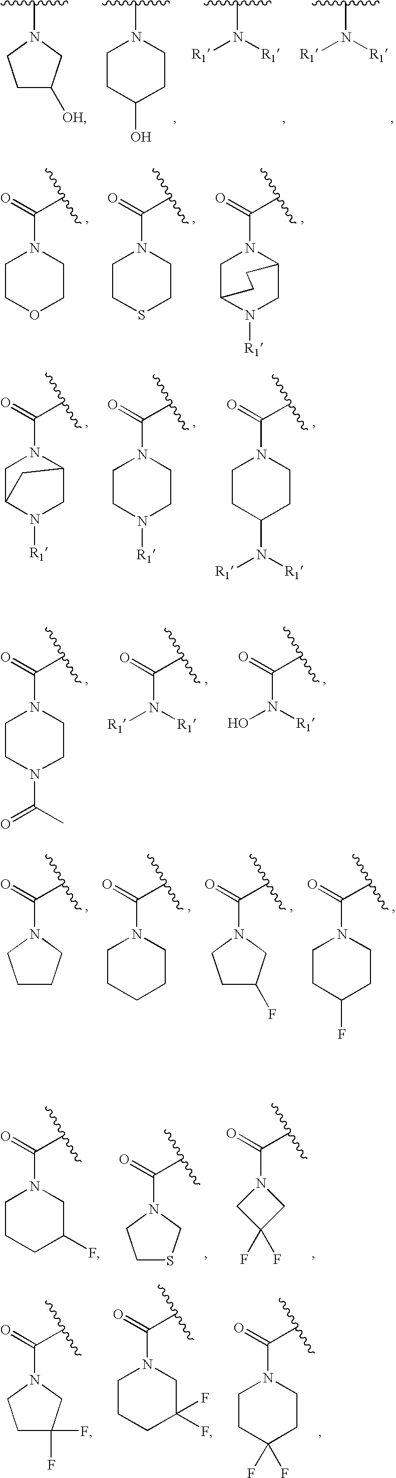

In another preferred embodiment, the solubilizing group is a moiety of the formula: —(CH₂)—O—R¹⁰¹, wherein n and R¹⁰¹ are as defined above.

In another preferred embodiment, the solubilizing group is a moiety of the formula: —(CH₂)—C(O)—R₁′, wherein n and R₁′ are as defined above.

In a more preferred embodiment, a solubilizing group is selected from —(CH₂)—R¹⁰², wherein n is 0, 1 or 2; and R₁₀₂ is selected from

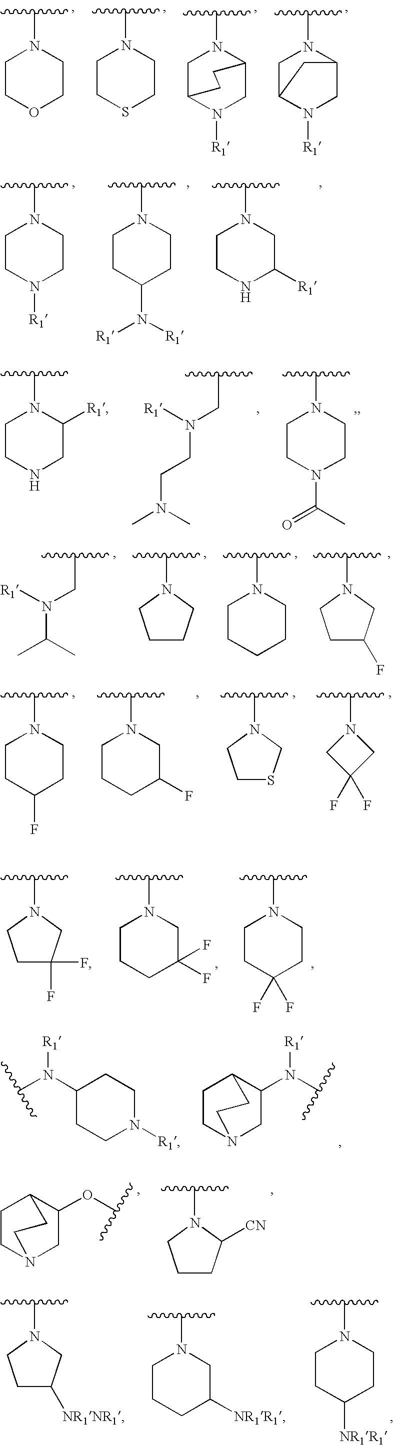

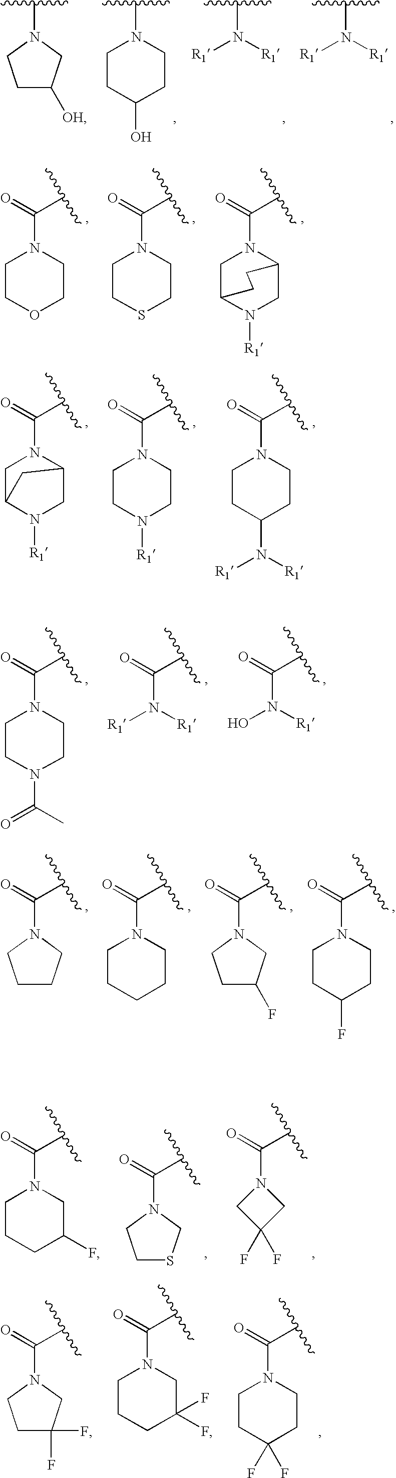

TABLE 4

| COM- | | | ED₅₀ | ED₅₀ | FOLD |
|---|---|---|---|---|---|
| POUND | | | FP | MS | ACT. |
| NO | [M + H]+ | STRUCTURE | ASSAY | ASSAY | MS |
| 1 | | 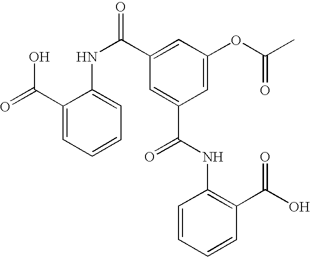 | A | NT | |
| 2 | | 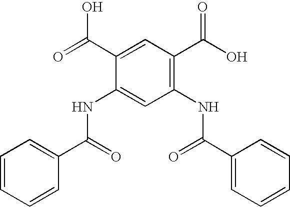 | D | NT | |
| 3 | | 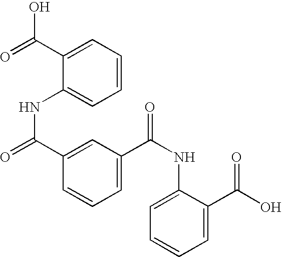 | B | NT | |
| 4 | | 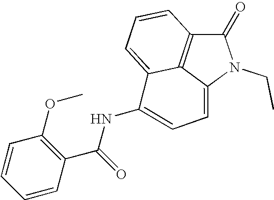 | N/A | NT | |
| 5 | | 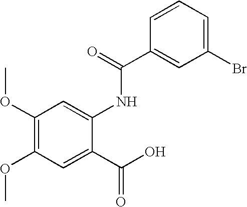 | B | NT | |
| 6 | | 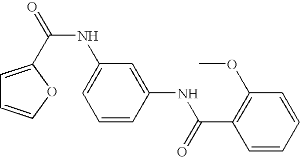 | D | NT | |
| 7 | 346 | 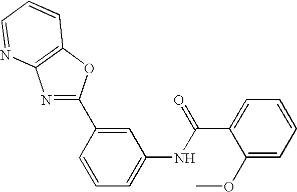 | A | NT | |
| 8 | | 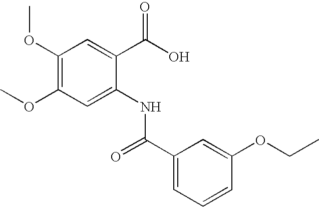 | B | NT | |
| 9 | | 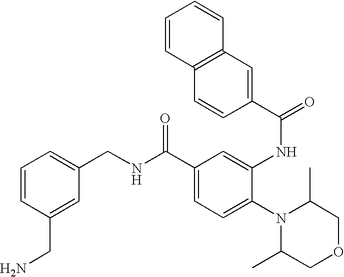 | C | NT | |
| 10 | | 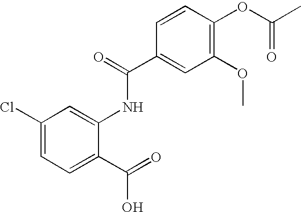 | D | NT | |
| 19 | 409.6 | 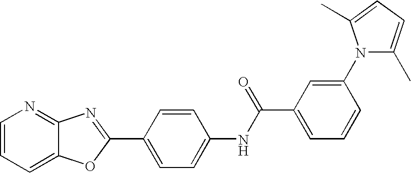 | D | D | C |
| 20 | 401.3 | 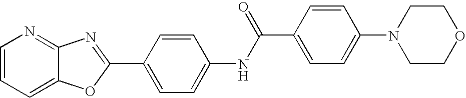 | D | D | C |
| 21 | 399.1 | 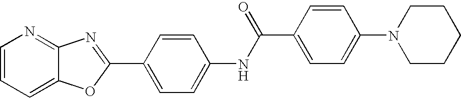 | D | D | C |
| 22 | 414.0 | 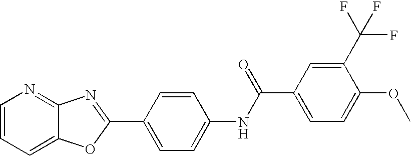 | D | D | C |
| 24 | 359.4 | 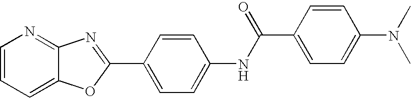 | D | D | C |
| 27 | 376.1 | 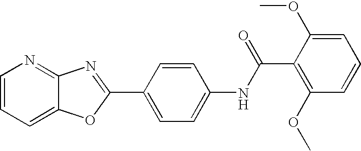 | D | D | C |
| 29 | 385.1 | 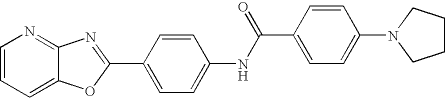 | D | D | C |
| 31 | 360.1 | 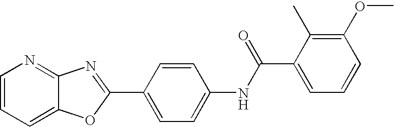 | D | D | C |
| 32 | 400.0 | 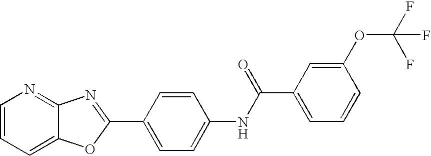 | D | D | C |
| 33 | 376.1 | 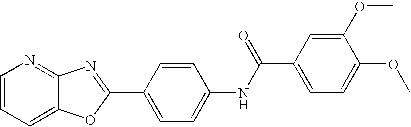 | D | D | C |
| 34 | 406.3 | 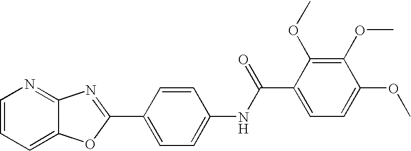 | D | D | C |
| 35 | 346.5 | 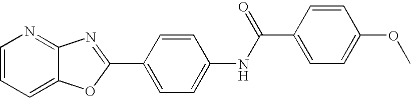 | D | D | C |
| 36 | 376.7 | 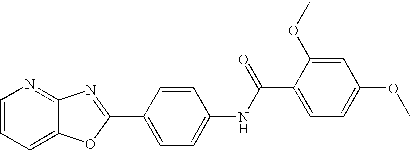 | D | D | C |
| 37 | 316.4 | 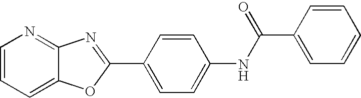 | D | D | C |
| 38 | 401.0 | 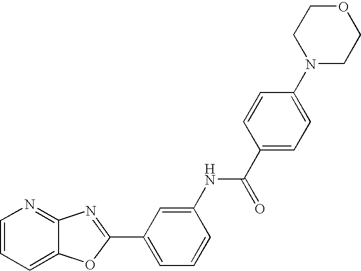 | D | D | C |
| 39 | 399.1 | 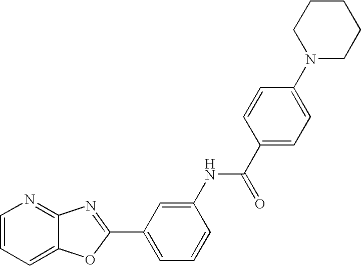 | D | D | C |
| 40 | 414.2 | 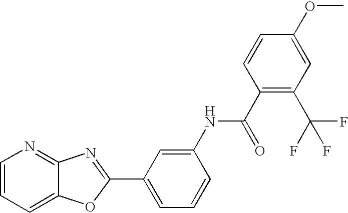 | D | D | C |
| 41 | 414.2 | 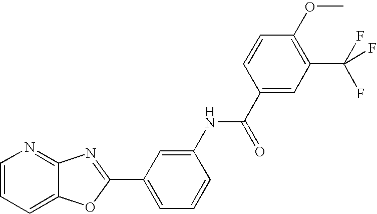 | D | D | C |
| 42 | 359.1 | 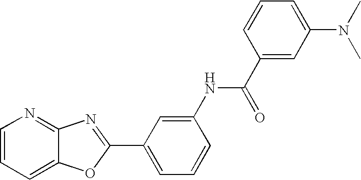 | A | A | B |
| 43 | 359.1 | 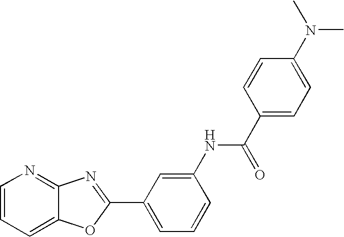 | A | A | B |
| 45 | 341.0 | 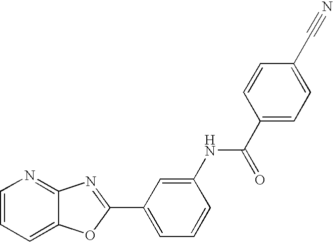 | D | D | C |
| 46 | 376.1 | 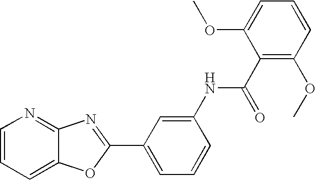 | D | D | C |
| 48 | 406.3 | 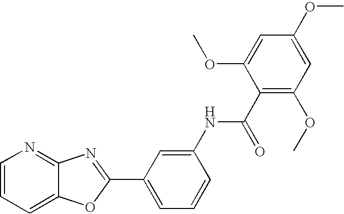 | D | D | C |
| 49 | 360.1 | 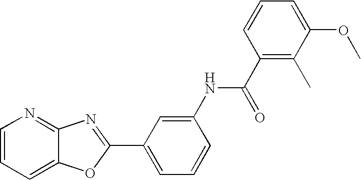 | D | A | B |
| 50 | 400.0 | 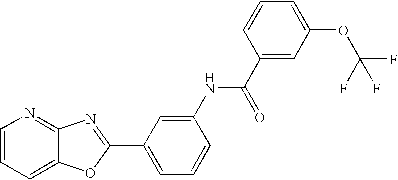 | NT | D | |
| 51 | 360.1 | 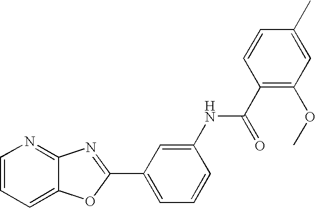 | A | A | B |
| 52 | 376.1 | 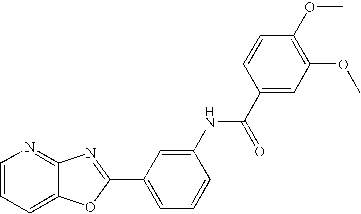 | A | A | B |
| 53 | 406.1 | 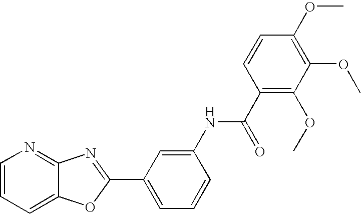 | D | D | C |
| 54 | 346.4 | 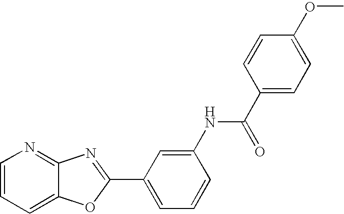 | NT | D | |
| 55 | 376.1 | 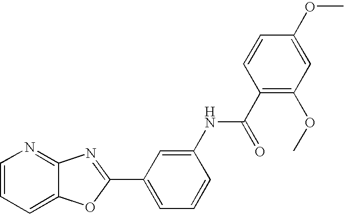 | A | A | B |
| 56 | 316.0 | 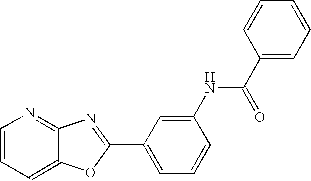 | D | A | B |
| 57 | 407.1 | 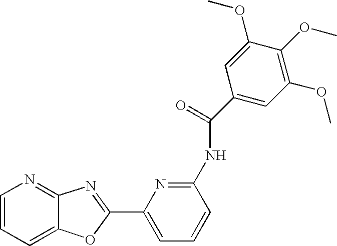 | | A | |
| 58 | 377.1 | 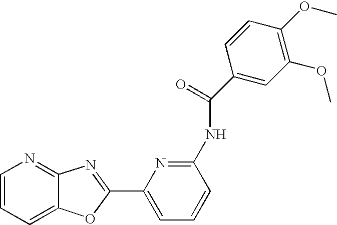 | | NA | |
| 59 | 318.1 | 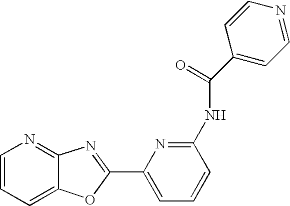 | | NA | |
| 60 | 413.1 | 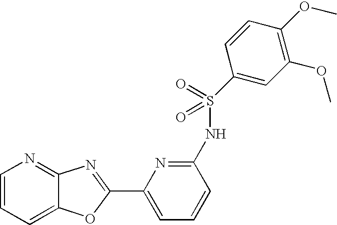 | | A | |
| 61 | 413.1 | 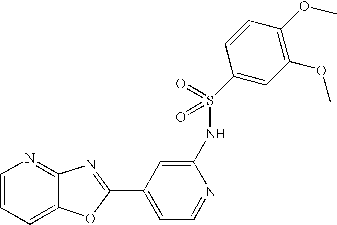 | | NA | |
| 62 | 349.0 | 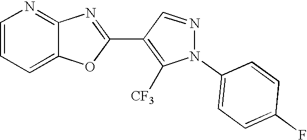 | | NA | |
| 63 | 377.1 | 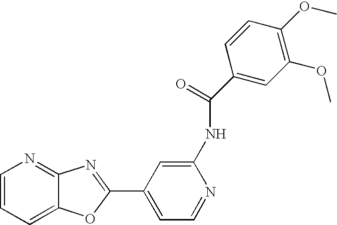 | | A | |
| 64 | 360.1 | 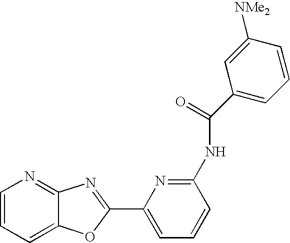 | | A | |
| 65 | 383.0 | 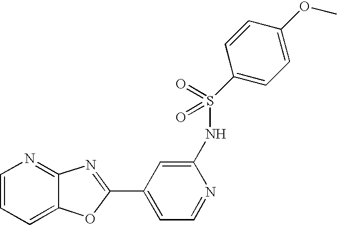 | | NA | |
| 66 | 407 | 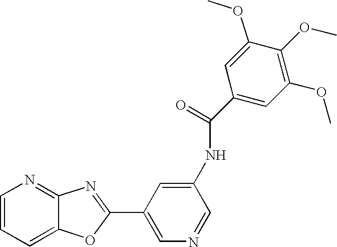 | | A | |
| 67 | 377 | 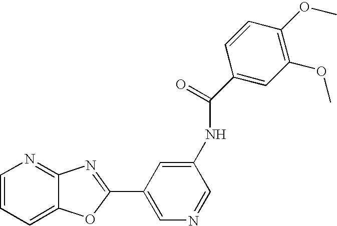 | | A | |
| 68 | 360 | 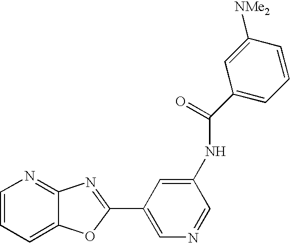 | | A | |
| 69 | 377.1 | 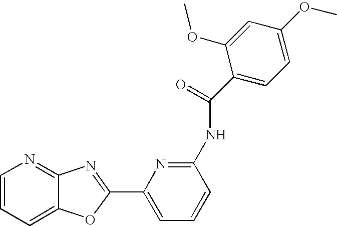 | | A | B |
| 70 | 360.1 | 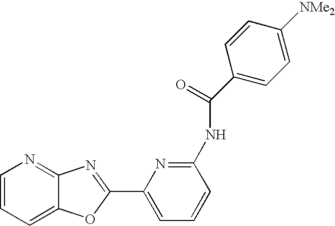 | | A | B |
| 71 | 376.1 | 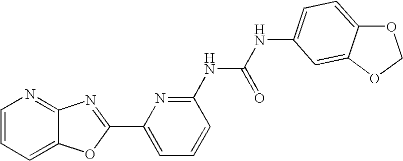 | | A | B |
| 72 | 422.1 | 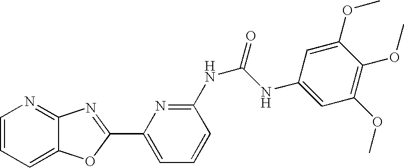 | | NT | |
| 73 | 377 | 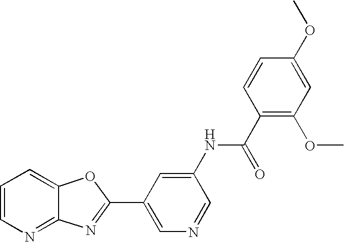 | | NT | |
| 74 | 412 | 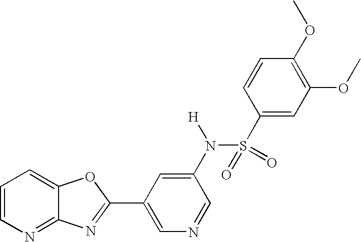 | | D | C |
| 75 | 407.1 | 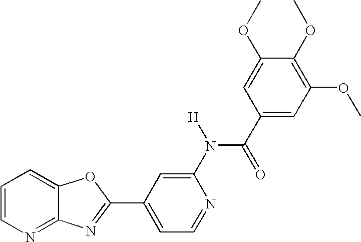 | | A | B |
| 76 | 360.1 | 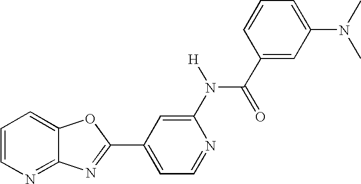 | | A | B |
| 77 | 376.1 | 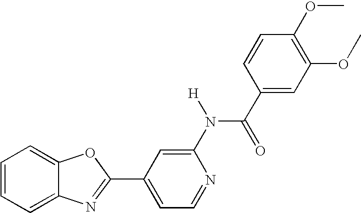 | | A | B |
| 78 | 445.1 | 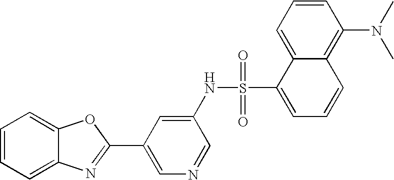 | | | |
| 79 | 338 | 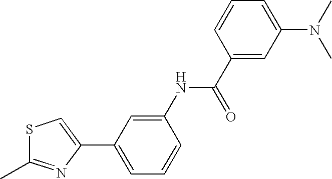 | | D | C |
| 80 | 355 | 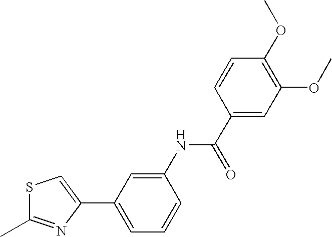 | | A | B |
| 81 | 354.5 | 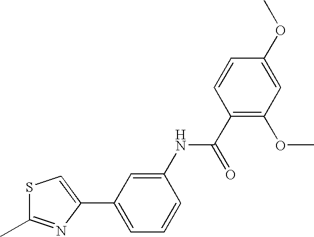 | | A | B |
| 82 | 402 | 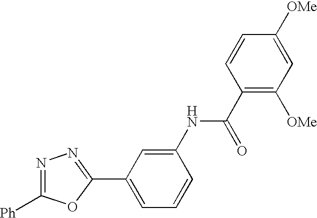 | | C | |
| 83 | 355 | 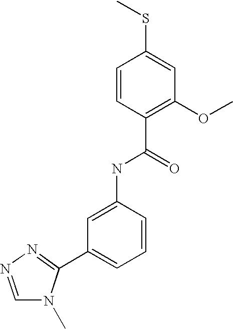 | | A | B |
| 84 | 417 | 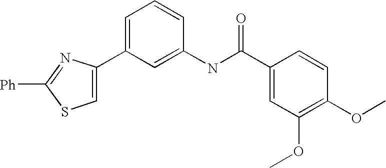 | | A | B |
| 85 | 335.1 | 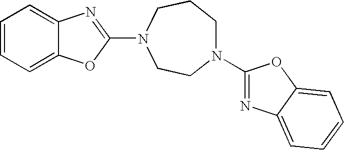 | | D | C |
| 86 | 375.1 | 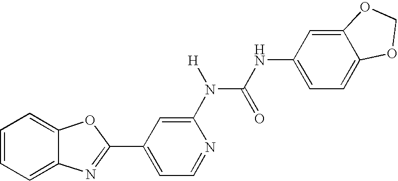 | | D | C |
| 87 | 375.1 | 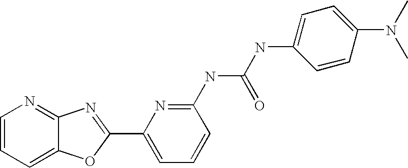 | | D | C |
| 88 | 382.1 | 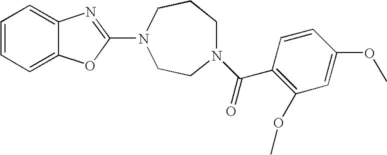 | | D | C |
| 89 | 365.1 | 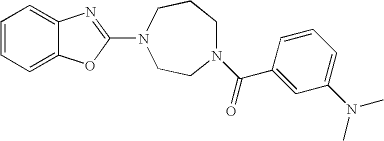 | | D | C |
| 90 | 381.1 | 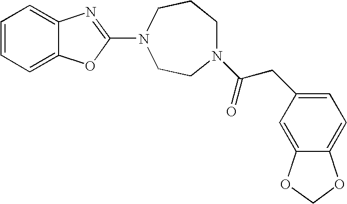 | | | |
| 91 | 412.1 | 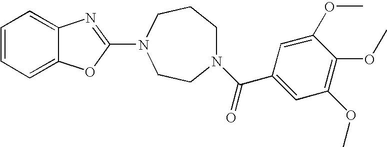 | | D | C |
| 92 | 308.1 | 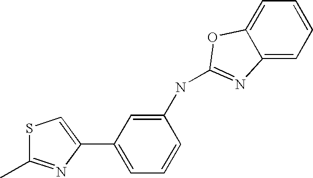 | | A | B |
| 93 | 329.1 | 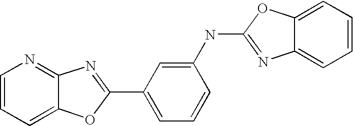 | | A | B |
| 94 | 434.1 | 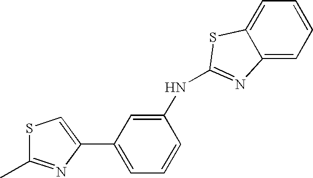 | | A | B |
| 95 | 345.1 | 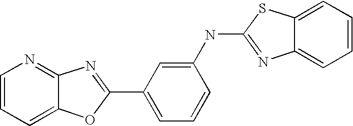 | | A′ | B |
| 96 | 376.1 | 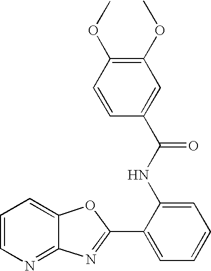 | | A′ | B |
| 97 | 359.1 | 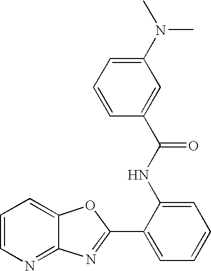 | | A′ | B |
| 98 | 408.1 | 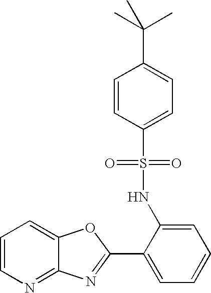 | | D | C |
| 99 | 391 | 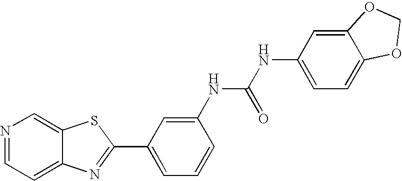 | | A′ | A |
| 100 | 376 | 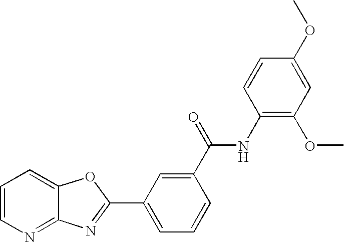 | | A | B |
| 101 | 376 | 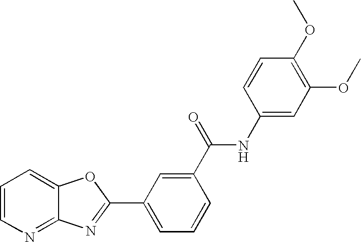 | | A | A |
| 102 | 406 | 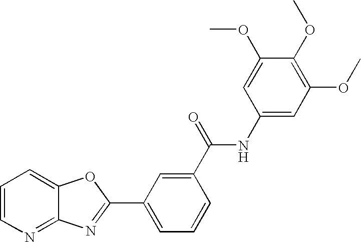 | | A | A |
| 103 | 374 | 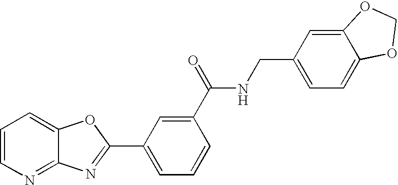 | | D | C |
| 104 | 346.1 | 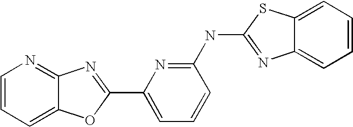 | | A′ | B |
| 105 | 330.1 | 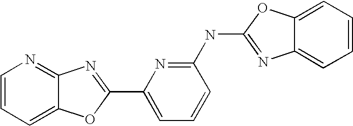 | | A′ | B |
| 106 | 406.1 | 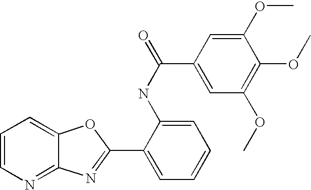 | | A′ | B |
| 107 | 488 | 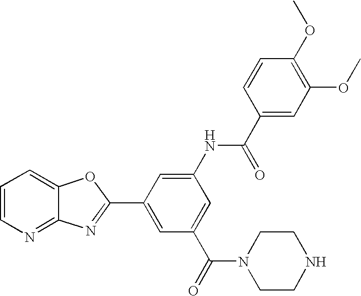 | | A | B |
| 108 | 324 | 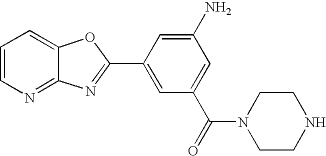 | | NA | |
| 109 | 401 | 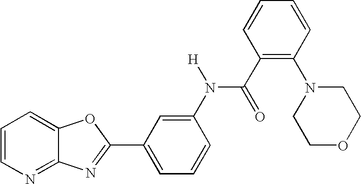 | | A | B |
| 110 | 381 | 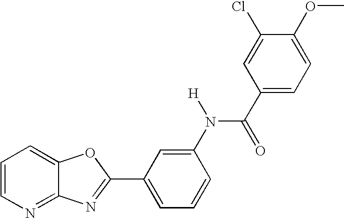 | | C | |
| 111 | 359 | 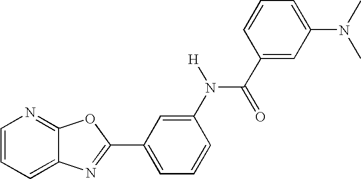 | | A | B |
| 112 | 376 | 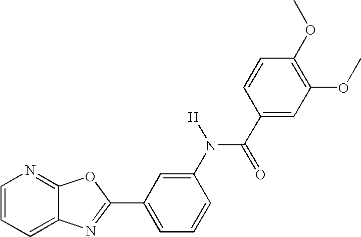 | | A | B |
| 113 | 375 | 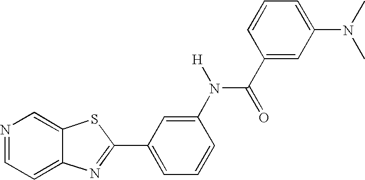 | | A | B |
| 114 | 392 | 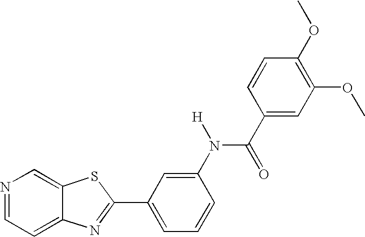 | | A′ | A |
| 115 | 422 | 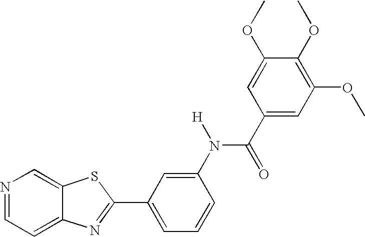 | | A | A |
| 116 | 386 | 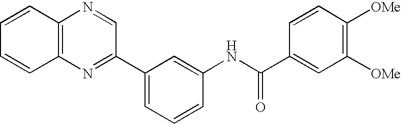 | | C | |
| 117 | 388 | 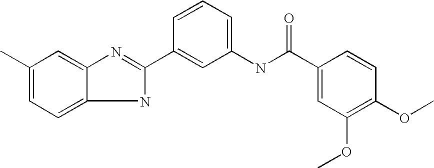 | | A | A |
| 118 | 410 | 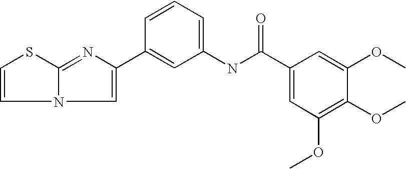 | | A | A |
| 119 | 375 | 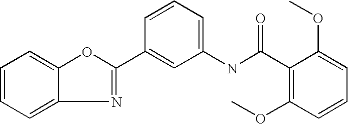 | | A | B |
| 120 | 391 | 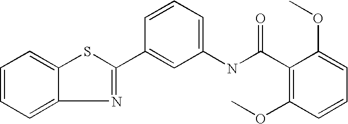 | | NT | |
| 121 | 414 | 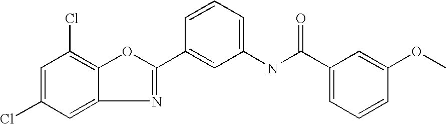 | | D | C |
| 122 | 417 | 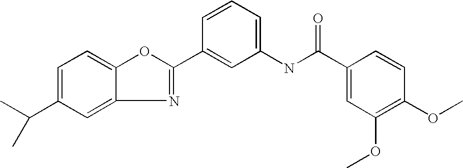 | | C | |
| 123 | 474 | 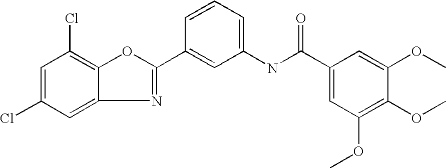 | | NT | |
| 124 | 391 | 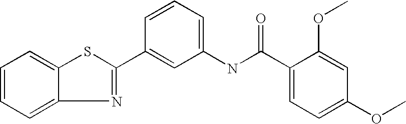 | | A | B |
| 125 | 433 | 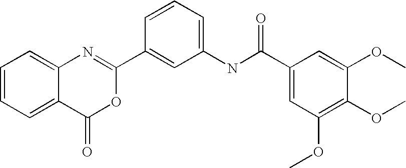 | | B | B |
| 126 | 374 | 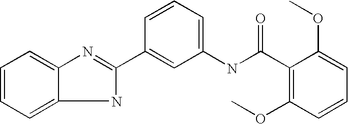 | | A | B |
| 127 | 355 | 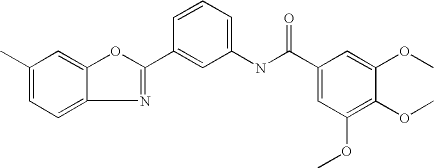 | | A | A |
| 128 | 388 | 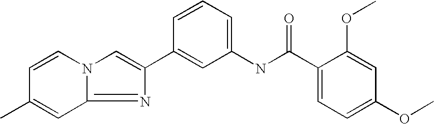 | | A | B |
| 129 | 418 | 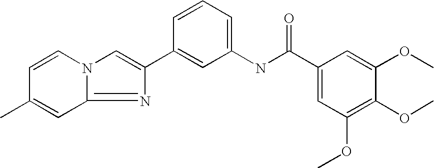 | | A′ | A |
| 130 | 358 | 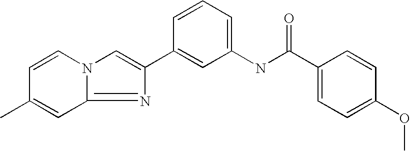 | | A | B |
| 131 | 418 | 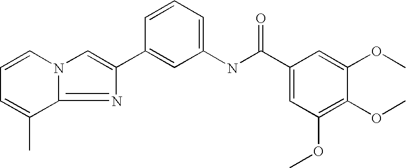 | | A | A |
| 132 | 350 | 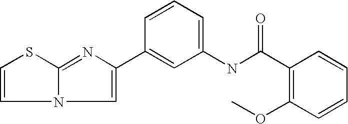 | | A | A |
| 133 | 480 | 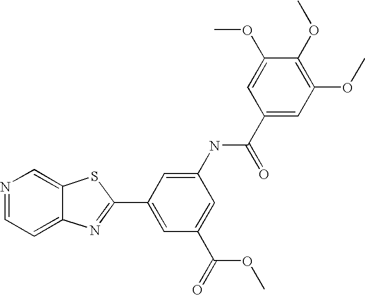 | | A′ | B |
| 134 | 466 | 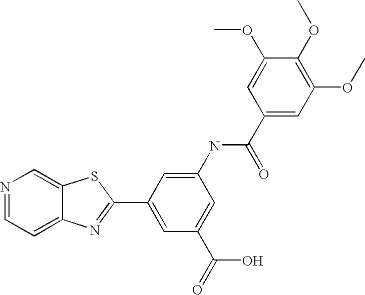 | | A′ | B |
| 135 | 452 | 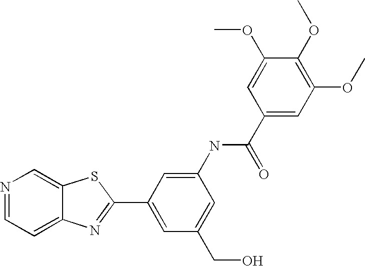 | | A′ | B |
| 136 | 434 | 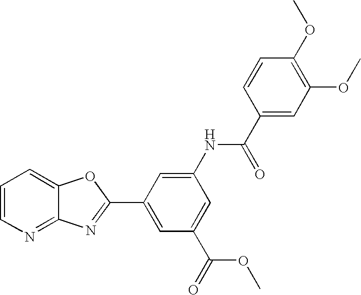 | | D | C |
| 137 | 420 | 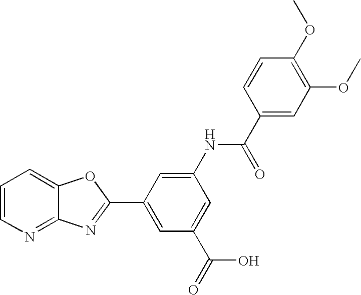 | | D | |
| 138 | 471 | 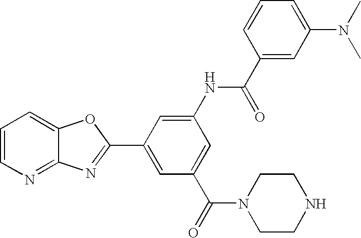 | | A | B |
| 139 | 488 | 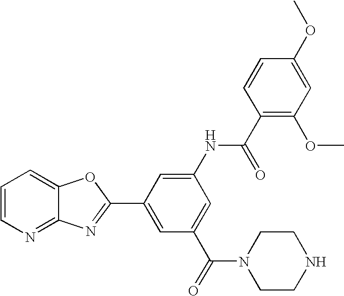 | | A | B |
| 141 | 374.1 | 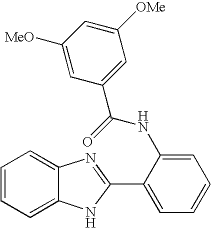 | | A′ | B |
| 142 | 374.1 | 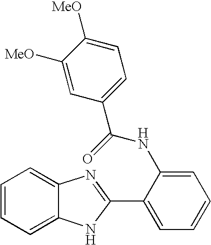 | | A′ | A |
| 143 | 410.1 | 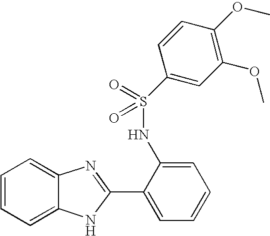 | | D | |
| 144 | 427.1 | 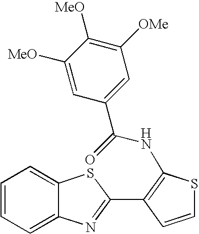 | | D | C |
| 145 | 397.1 | 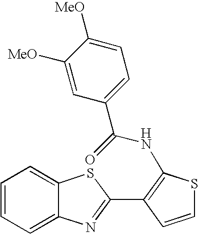 | | A′ | B |
| 146 | 397.1 | 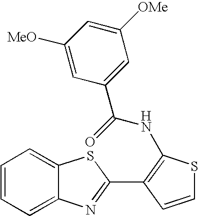 | | A′ | B |
| 147 | 392.1 | 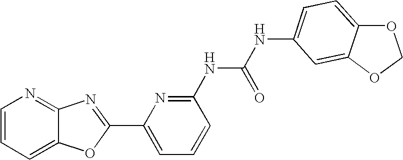 | | D | |
| 148 | 405.2 | 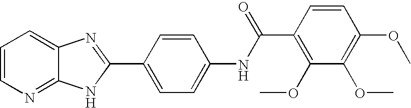 | | D | |
| 149 | 359.0 | 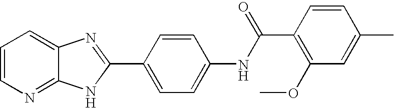 | | A′ | B |
| 150 | 375.0 | 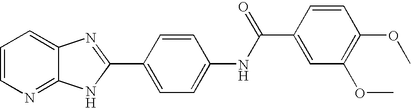 | | A′ | B |
| 151 | 375.0 | 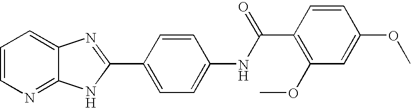 | | D | C |
| 152 | 392.1 | 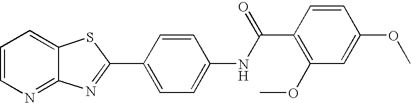 | | D | C |
| 153 | 490 | 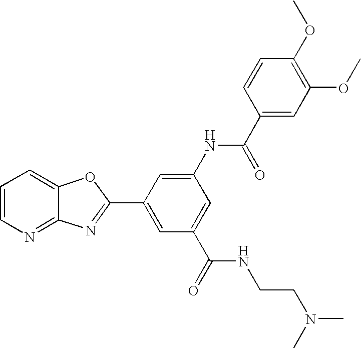 | | A | B |
| 154 | 473 | 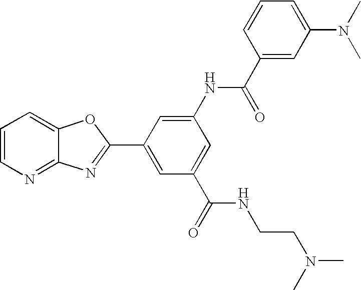 | | C | A |
| 155 | 490 | 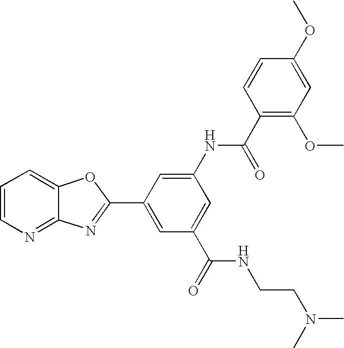 | | A | B |
| 156 | 433 | 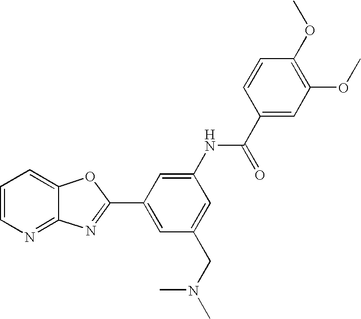 | | A | B |
| 157 | 416 | 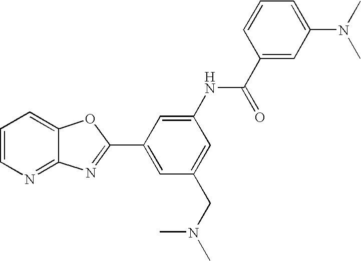 | | D | B |
| 158 | 433 | 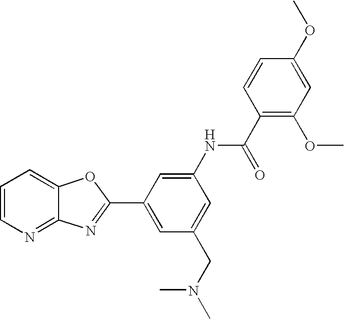 | | A | B |
| 159 | 474 | 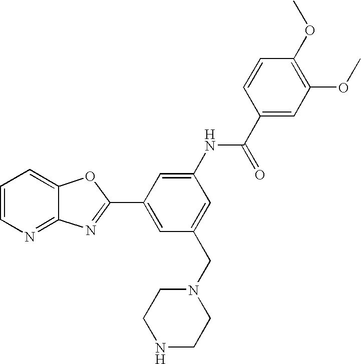 | | A | B |
| 160 | 457 | 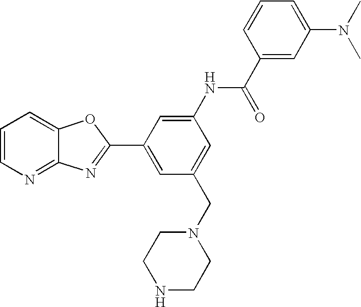 | | B | A |
| 161 | 474 | 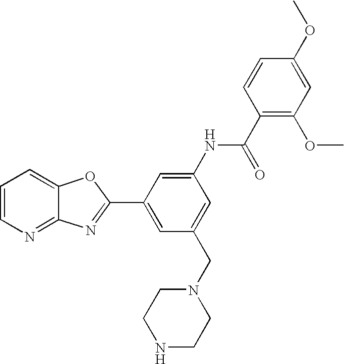 | | A | A |
| 162 | 392.1 | 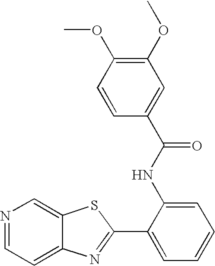 | | A′ | B |
| 163 | 422.1 | 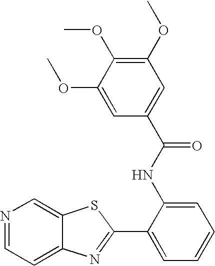 | | A′ | B |
| 164 | 488 | 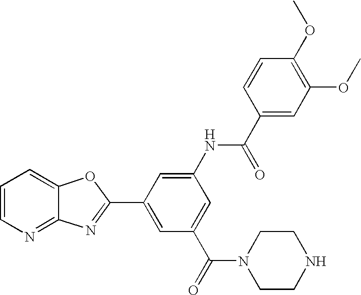 | | A | B |
| 165 | 416 | 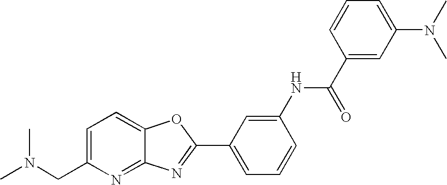 | | D | A |
| 166 | 457 | 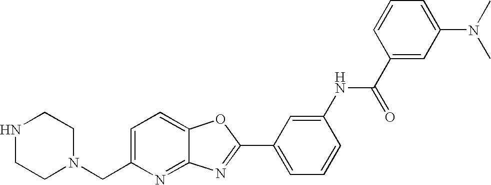 | | A | B |
| 167 | 373 | 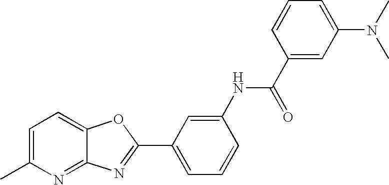 | | A | B |
| 168 | 388.1 | 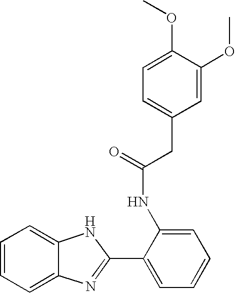 | | NA | C |
| 169 | 343.1 | 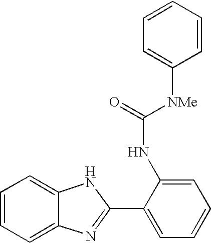 | | A′ | B |
| 174 | 479 | 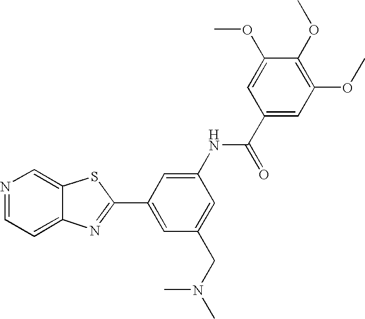 | | B | A |
| 175 | 323.1 | 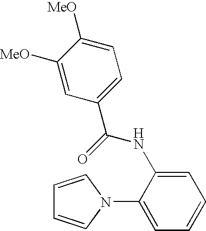 | | B | B |
| 176 | 354.1 | 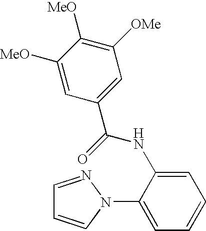 | | B | B |
| 177 | 324.1 | 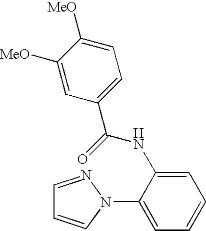 | | D | B |
| 178 | 437 | 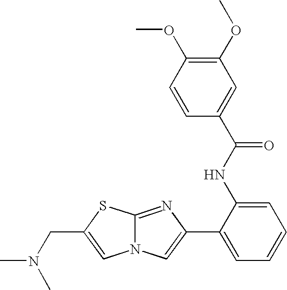 | | A | A |
| 179 | 467 | 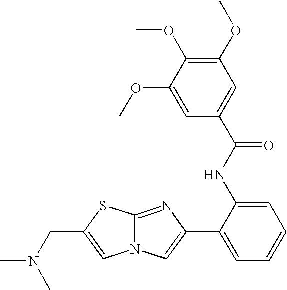 | | A′ | A |
| 180 | 358.0 | 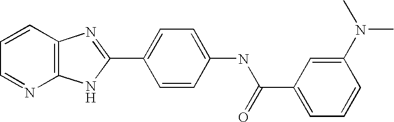 | | A | B |
| 181 | 405.0 | 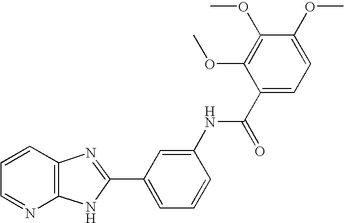 | | A′ | A |
| 182 | 359.0 | 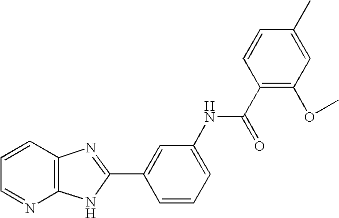 | | A′ | A |
| 183 | 358.0 | 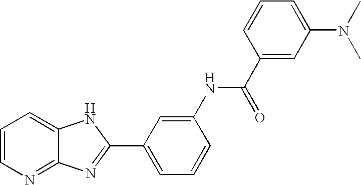 | | A | A |
| 184 | 375.0 | 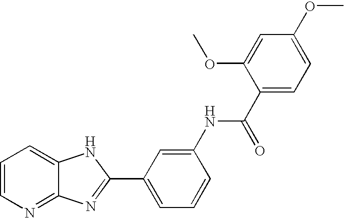 | | A′ | B |
| 185 | 374.9 | 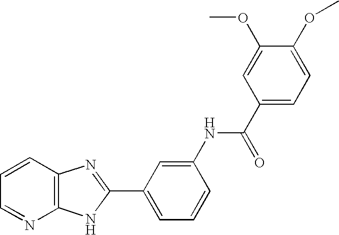 | | A′ | A |
| 186 | 405.3 | 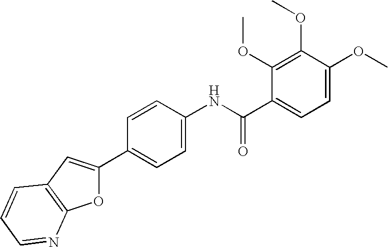 | | NA | C |
| 187 | 358.9 | 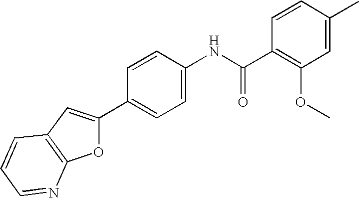 | | A | B |
| 188 | 358.9 | 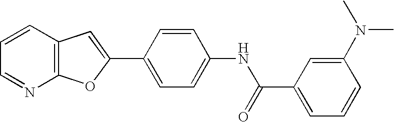 | | NA | C |
| 189 | 375.2 | 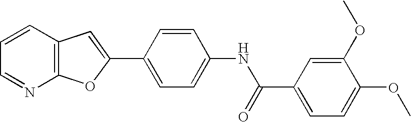 | | NA | C |
| 190 | 375.3 | 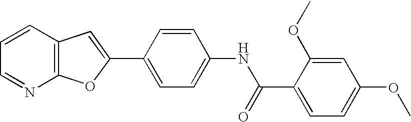 | | A′ | B |
| 191 | 375.0 | 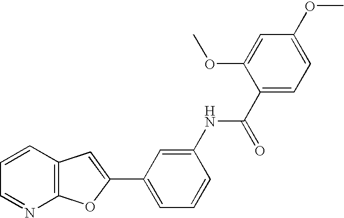 | | A′ | B |
| 192 | 375.0 | 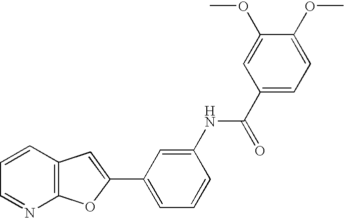 | | A | B |
| 193 | 358.0 | 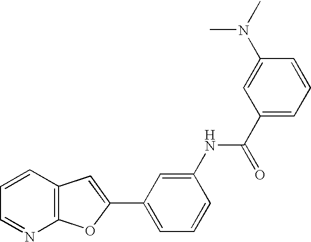 | | NA | C |
| 194 | 422.3 | 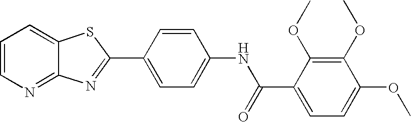 | | NA | C |
| 195 | 375.9 | 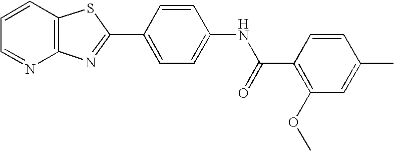 | | NA | C |
| 196 | 374.9 | 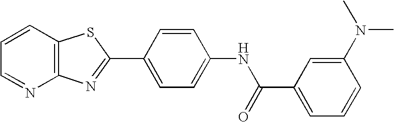 | | A | B |
| 197 | 391.1 | 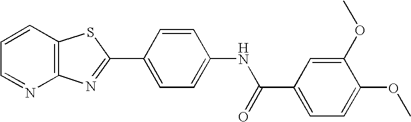 | | A′ | B |
| 198 | 422.4 | 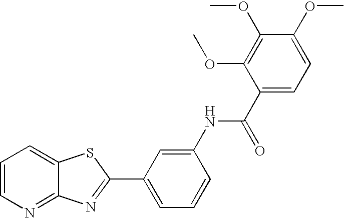 | | A | B |
| 199 | 375.9 | 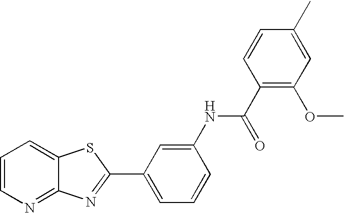 | | A′ | B |
| 200 | 375.4 | 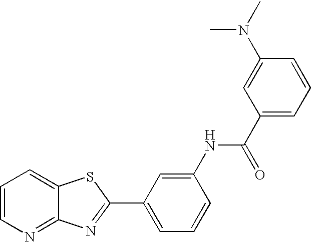 | | A′ | B |
| 201 | 391.9 | 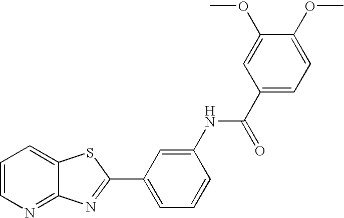 | | A | B |
| 202 | 392.4 | 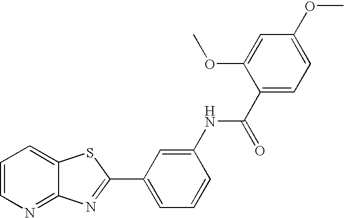 | | A′ | B |
| 203 | 380 | 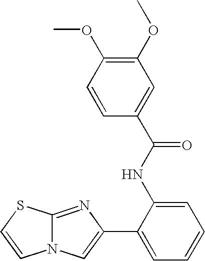 | | A′ | B |
| 204 | 410 | 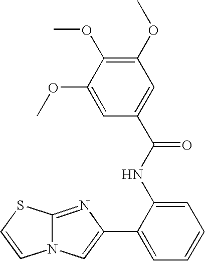 | | A′ | A |
| 205 | 437 | 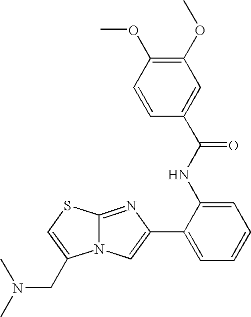 | | A | A |
| 206 | 467 | 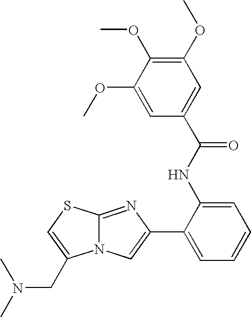 | | A | A |
| 207 | 478 | 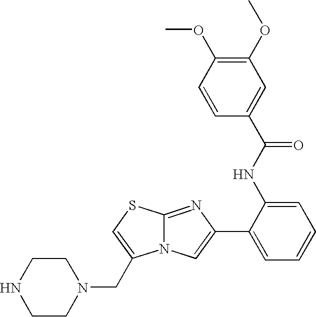 | | A | A |
| 208 | 508 | 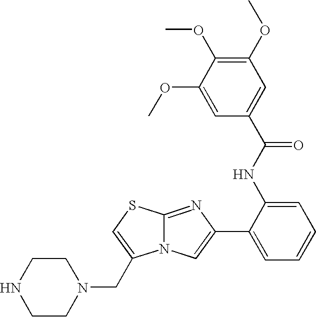 | | A | A |
| 209 | 479 | 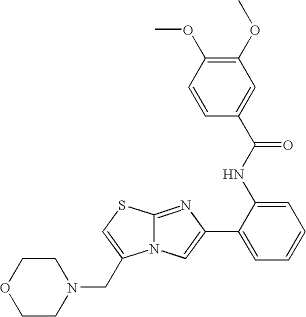 | | A | A |
| 210 | 509 | 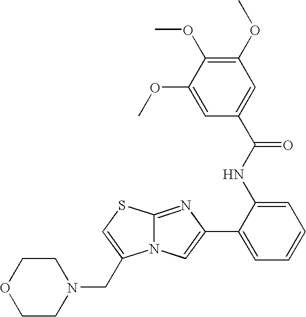 | | A | B |
| 211 | | 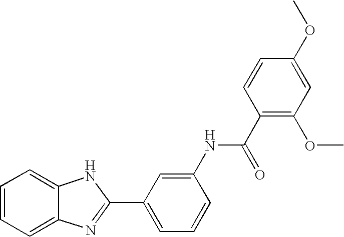 | | A′ | B |
| 212 | 362 | 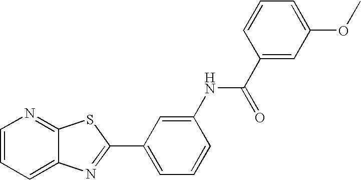 | | A | B |
| 213 | 392 | 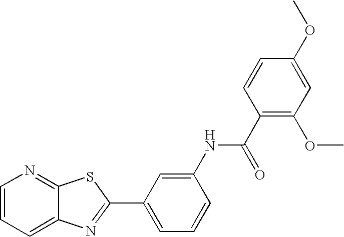 | | A′ | B |
| 214 | 392 | 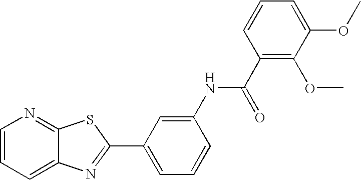 | | A | B |
| 215 | 392 | 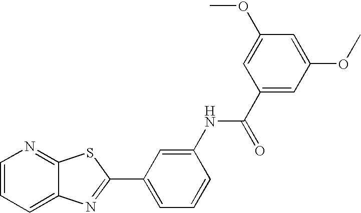 | | A′ | B |
| 216 | 362 | 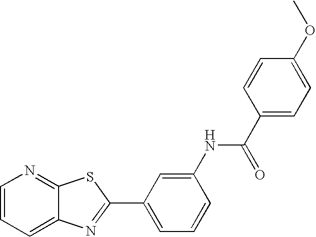 | | A′ | B |
| 217 | 422 | 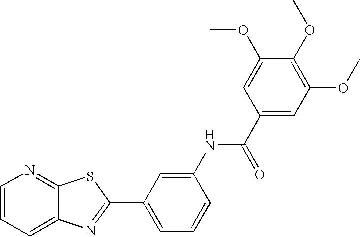 | | A′ | A |
| 218 | 405 | 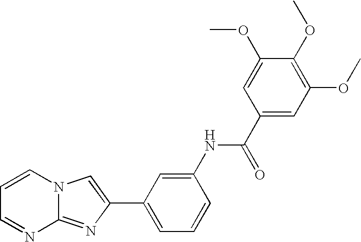 | | A′ | A |
| 219 | 419 | 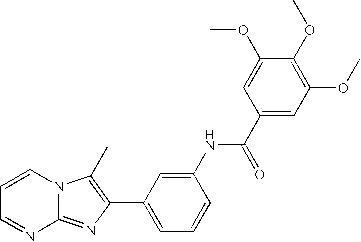 | | A | A |
| 220 | 423 | 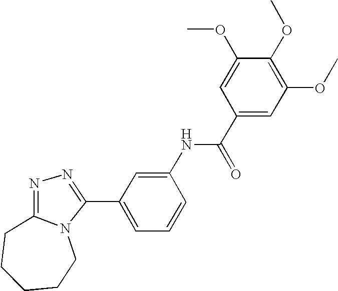 | | NA | C |
| 221 | 321.4 | 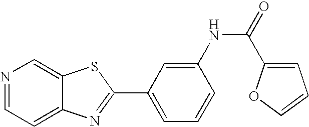 | | A | B |
| 222 | 366.8 | 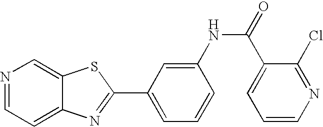 | | A | B |
| 223 | 337.4 | 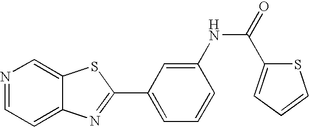 | | A′ | B |
| 225 | 332.4 | 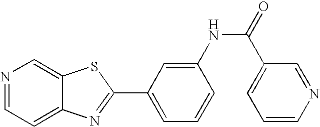 | | NA | C |
| 226 | 412.5 | 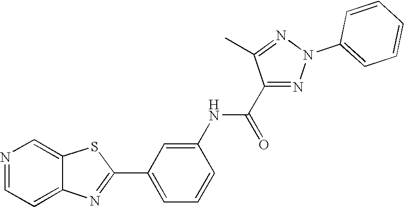 | | | |
| 227 | 333.4 | 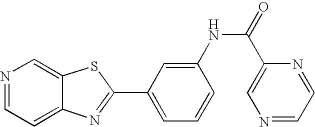 | | NA | C |
| 228 | 391.5 | 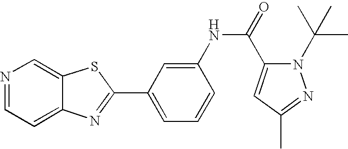 | | A | A |
| 229 | 418.5 | 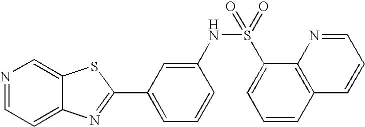 | | NA | C |
| 230 | 459.5 | 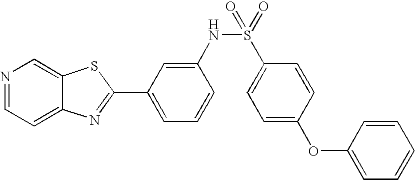 | | NA | C |
| 231 | 425.5 | 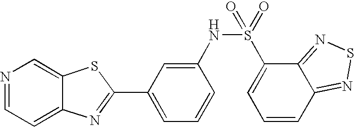 | | NA | C |
| 232 | 423.5 | 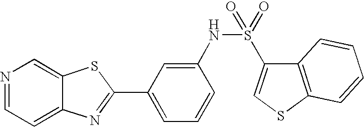 | | NA | C |
| 233 | 449.5 | 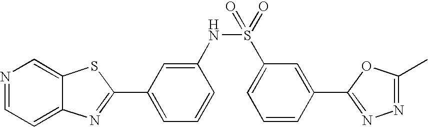 | | NA | C |
| 235 | 436.3 | 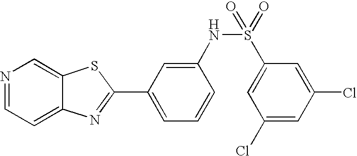 | | NA | C |
| 236 | 423.5 | 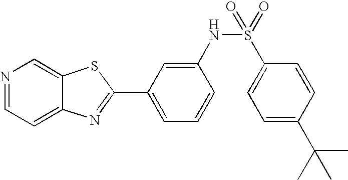 | | NA | C |
| 237 | 450.5 | 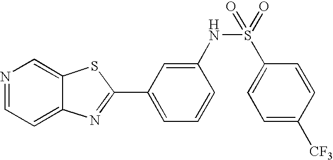 | | NA | C |
| 238 | 480.5 | 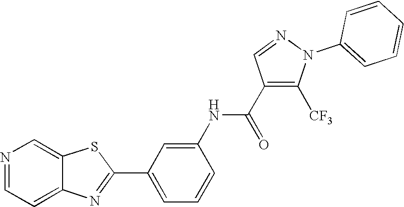 | | A′ | B |
| 239 | 466.5 | 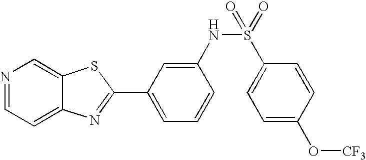 | | A | B |
| 240 | 392.4 | 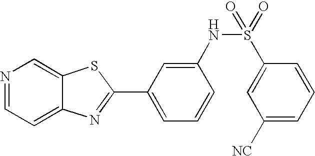 | | NA | C |
| 241 | 397.2 | 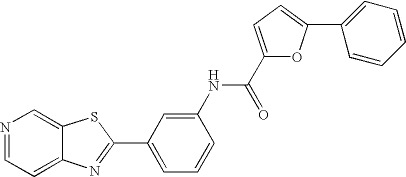 | | A′ | B |
| 244 | 332.4 | 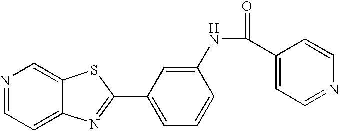 | | NA | C |
| 245 | 423.5 | 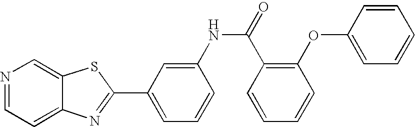 | | A′ | B |
| 246 | 391.5 | 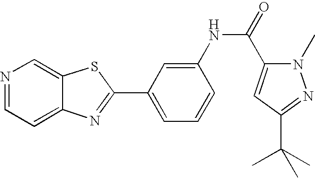 | | A′ | B |
| 247 | 413.5 | 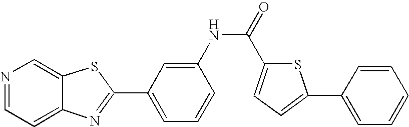 | | NA | C |
| 248 | 467.4 | 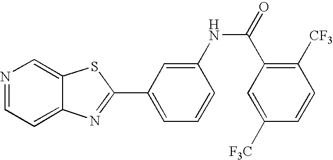 | | NA | C |
| 249 | 395.9 | 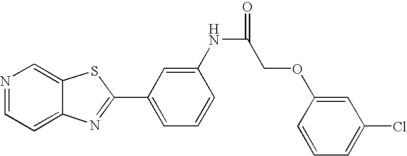 | | NA | C |
| 250 | 385.5 | 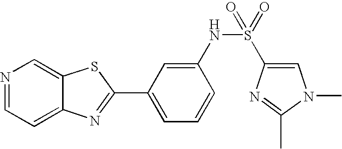 | | NA | C |
| 251 | 425.5 | 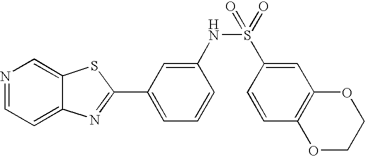 | | NA | C |
| 252 | 453.5 | 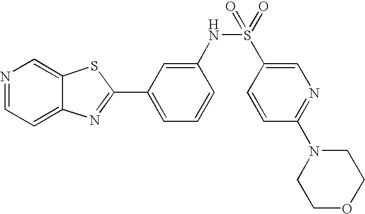 | | NA | C |
| 253 | 373.1 | 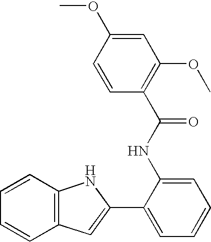 | | NA | C |
| 254 | 373 | 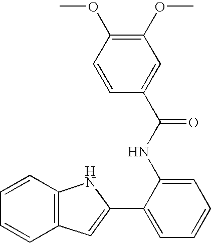 | | A′ | B |
| 255 | 403 | 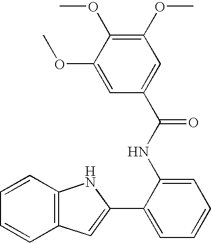 | | A′ | B |
| 256 | 356 | 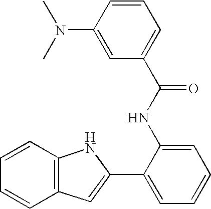 | | NA | C |
| 257 | 413.1 | 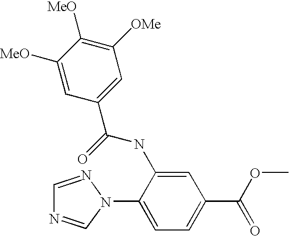 | | NA | C |
| 258 | 383.1 | 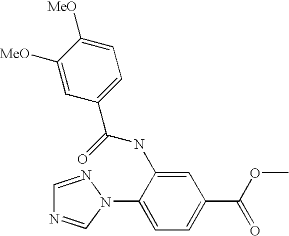 | | A | B |
| 259 | 405.1 | 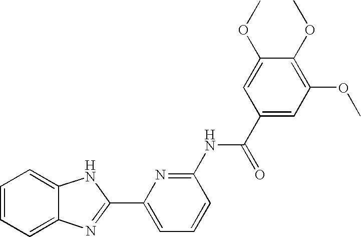 | | A′ | A |
| 260 | 375.1 | 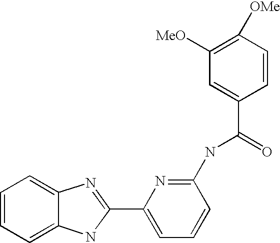 | | A′ | A |
| 261 | 375.1 | 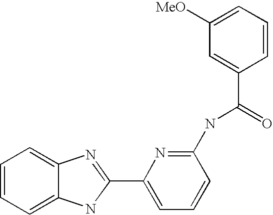 | | A′ | A |
| 262 | 374.1 | 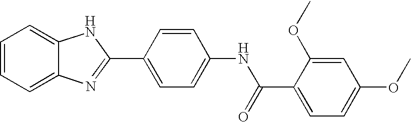 | | A′ | B |
| 263 | 404 | 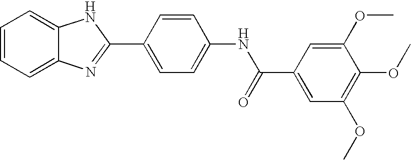 | | A | B |
| 264 | 357 | 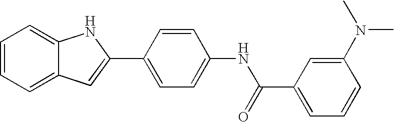 | | A′ | B |
| 265 | 374.1 | 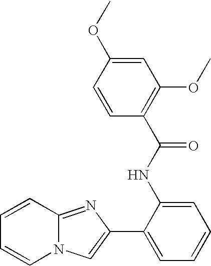 | | A | B |
| 266 | 374 | 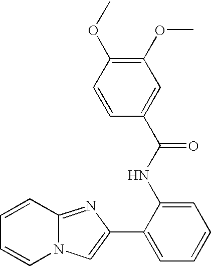 | | A′ | A |
| 267 | 404 | 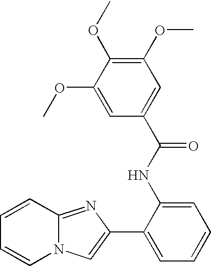 | | A′ | B |
| 268 | 357 | 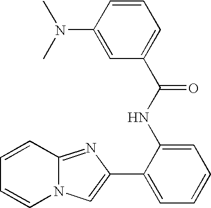 | | A′ | B |
| 270 | 478 | 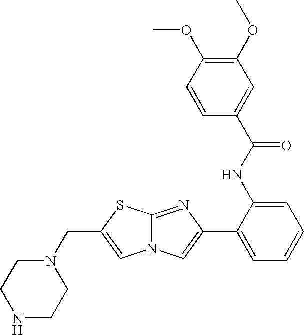 | | A | A |
| 271 | 508 | 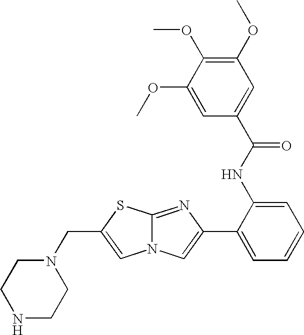 | | A′ | A |
| 272 | 392 | 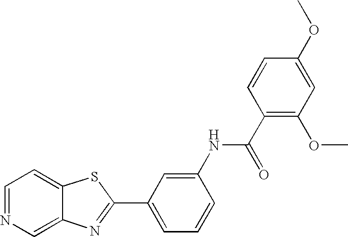 | | A′ | B |
| 273 | 422 | 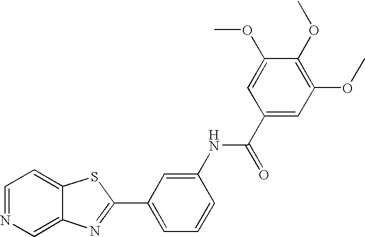 | | A | A |
| 276 | 375.1 | 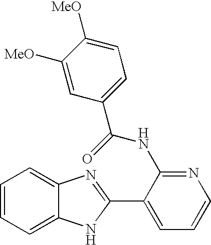 | | A′ | B |
| 280 | 381.5 | 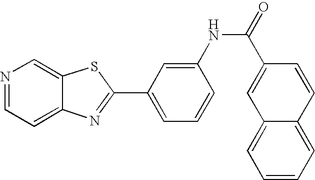 | | | |
| 282 | 386.5 | 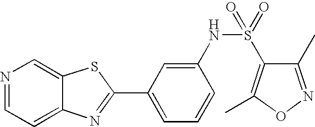 | | NA | C |
| 283 | 451.6 | 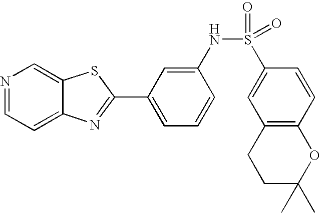 | | NA | C |
| 284 | 439.5 | 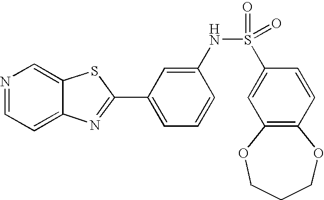 | | NA | C |
| 285 | 440.5 | 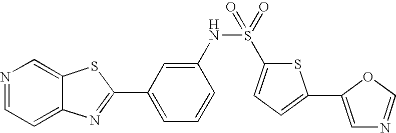 | | NA | C |
| 286 | 390.1 | 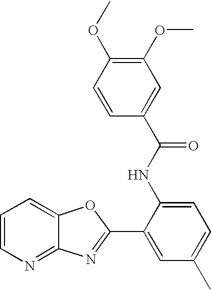 | | NA | C |
| 287 | 457.6 | 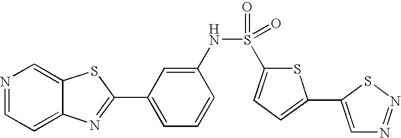 | | | |
| 288 | 424.5 | 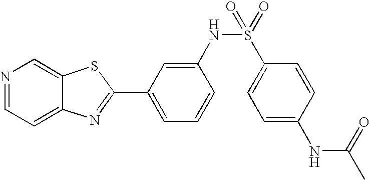 | | A | B |
| 289 | 427.5 | 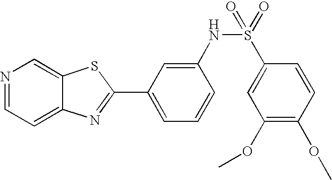 | | A | B |
| 290 | 445.5 | 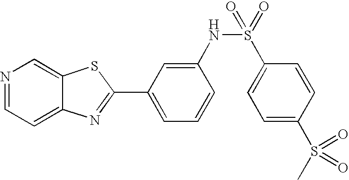 | | NA | C |
| 292 | 420.1 | 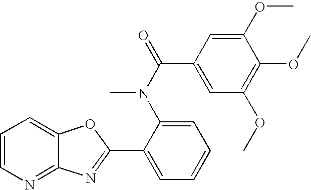 | | D | |
| 293 | 404.1 | 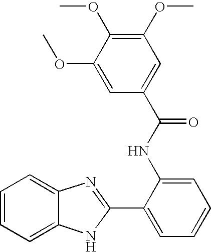 | | A′ | A |
| 294 | 374 | 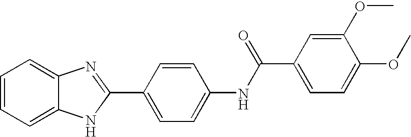 | | A′ | B |
| 295 | 252.1 | 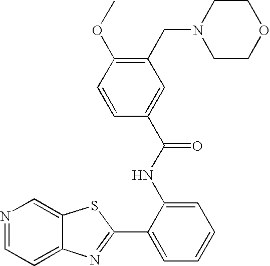 | | A′ | B |
| 296 | 376 | 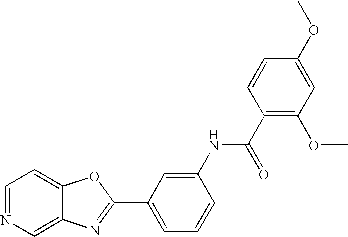 | | A | B |
| 297 | 376 | 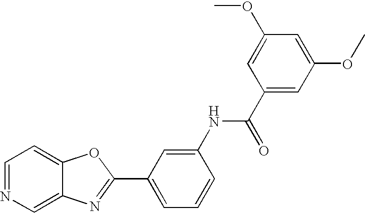 | | A | B |
| 298 | 406 | 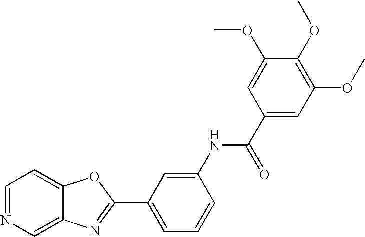 | | A | B |
| 299 | 359 | 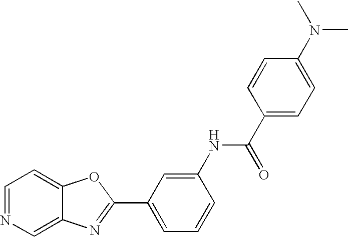 | | A | B |
| 303 | 437.5 | 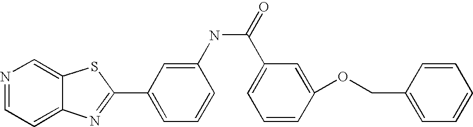 | | NA | |
| 304 | 336.4 | 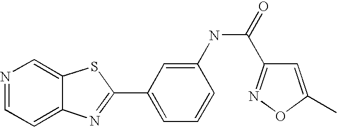 | | A′ | B |
| 305 | 414.5 | 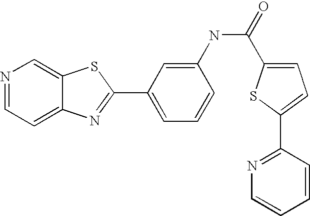 | | NA | |
| 306 | 424.5 | 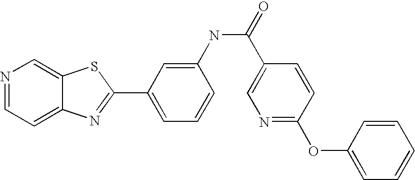 | | A | B |
| 307 | 382.4 | 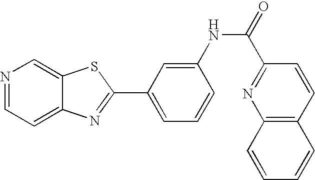 | | A′ | A |
| 308 | 400.3 | 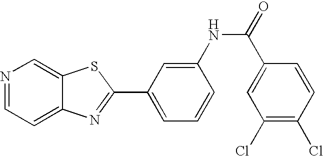 | | NA | |
| 309 | 367.4 | 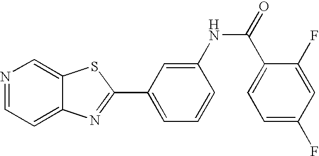 | | NA | |
| 310 | 387.5 | 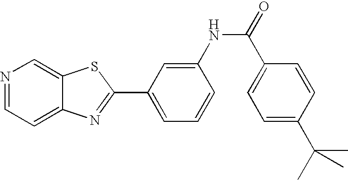 | | NA | |
| 311 | 359 | 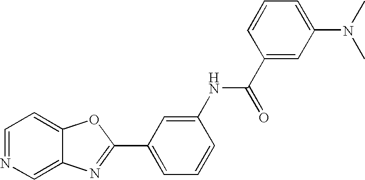 | | A | B |
| 313 | 418.1 | 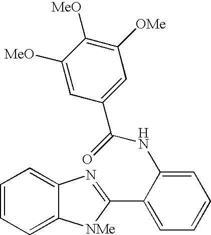 | | A | B |
| 314 | 388.1 | 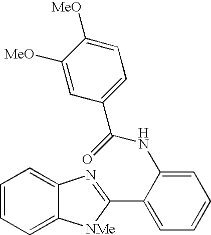 | | A | B |
| 315 | 388.1 | 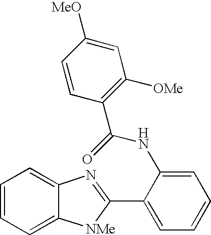 | | NA | |
| 317 | 392 | 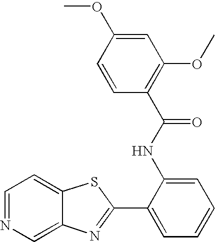 | | A′ | B |
| 318 | 392 | 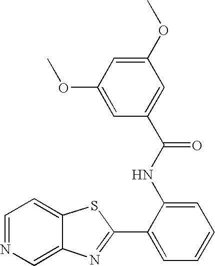 | | A | B |
| 319 | 422 | 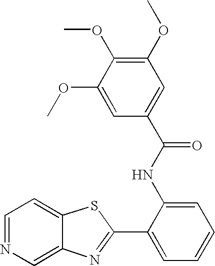 | | A′ | B |
| 320 | 375 | 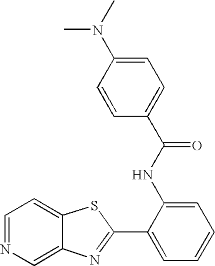 | | A′ | B |
| 321 | 375 | 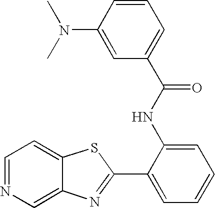 | | A′ | B |
| 322 | 392 | 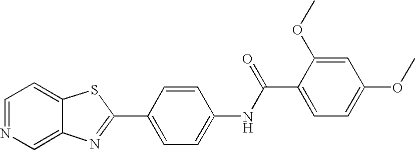 | | NA | |
| 323 | 392 | 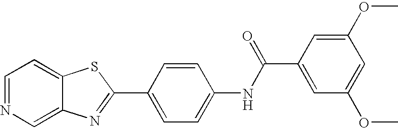 | | A | B |
| 324 | 422 | 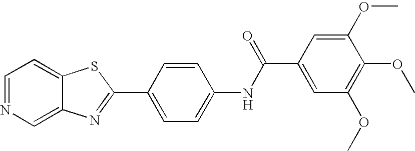 | | NA | |
| 325 | 375 | 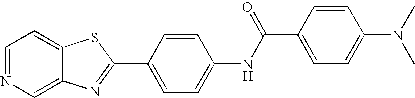 | | NA | |
| 326 | 478 | 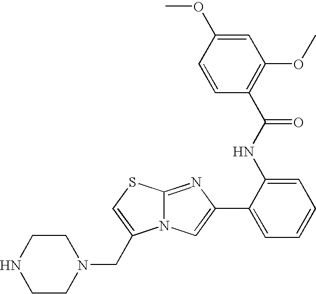 | | A | B |
| 327 | 461 | 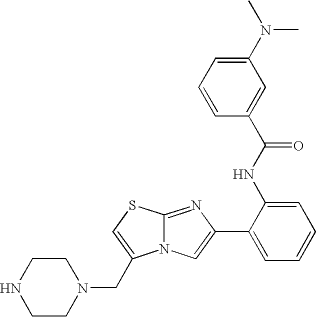 | | A | A |
| 328 | 418 | 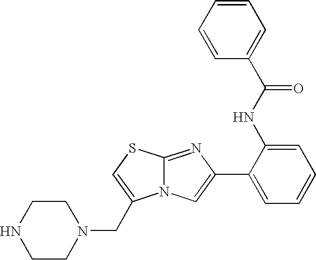 | | B | A |
| 329 | 462 | 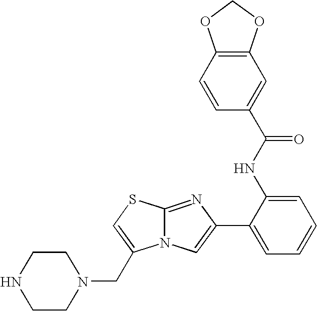 | | A | A |
| 330 | 443 | 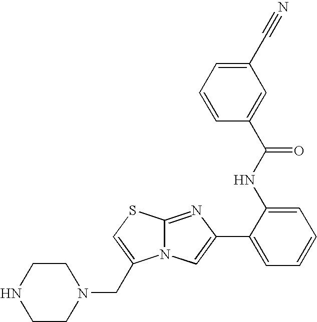 | | A | A |
| 331 | 335 | 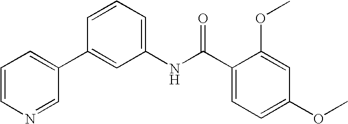 | | A | B |
| 332 | 335.1 | 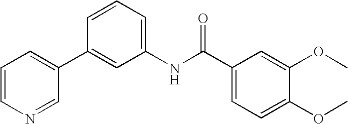 | | A | B |
| 333 | 365 | 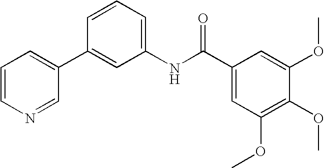 | | A | B |
| 334 | 340.1 | 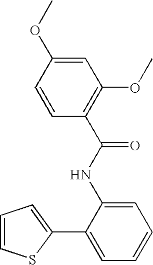 | | A | B |
| 335 | 340 | 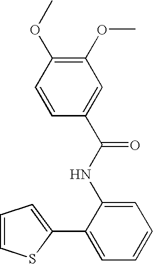 | | A | B |
| 336 10 | 370 | 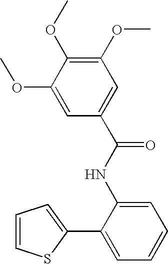 | | A | B |
| 337 | 443 | 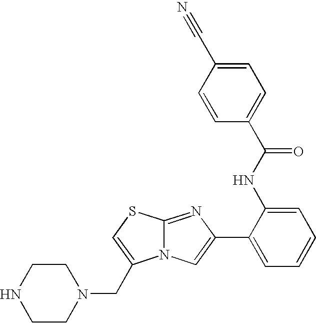 | | NA | |
| 338 | 458 | 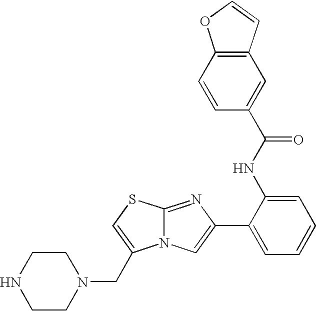 | | A | A |
| 339 | 376 | 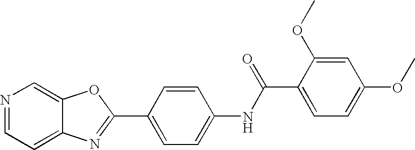 | | NA | |
| 340 | 376 | 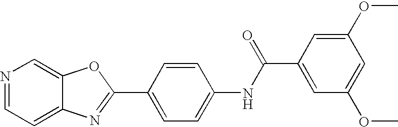 | | NA | |
| 341 | 406 | 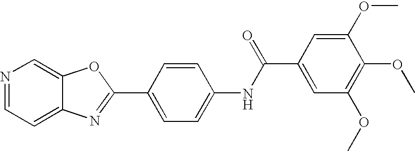 | | A′ | B |
| 342 | 359 | 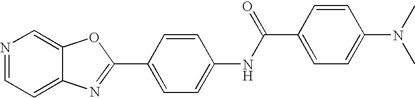 | | NA | |
| 343 | 406 | 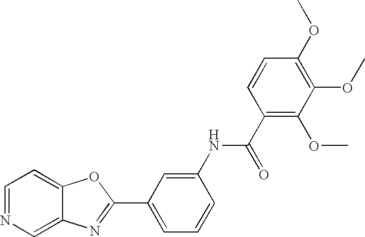 | | A | B |
| 344 | 375 | 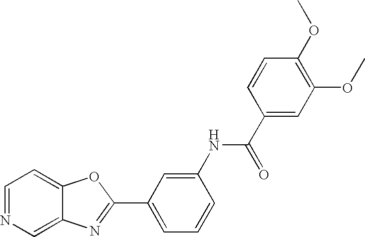 | | A′ | B |
| 345 | 376 | 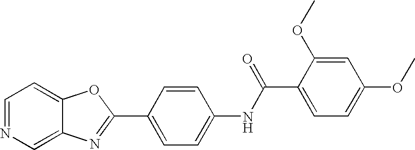 | | NA | |
| 346 | 376 | 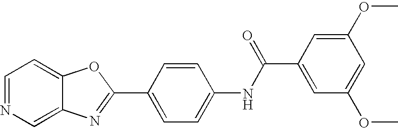 | | NA | |
| 347 | 406 | 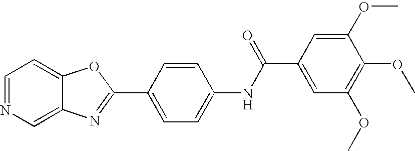 | | A′ | B |
| 348 | 359 | 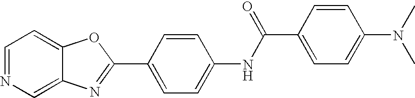 | | NA | |
| 349 | 375 | 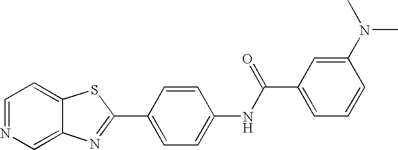 | | NA | |
| 350 | 431.1 | 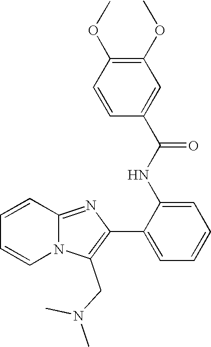 | | B | B |
| 351 | 461 | 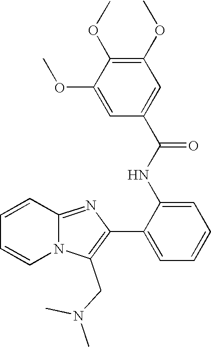 | | A | B |
| 359 | 472.1 | 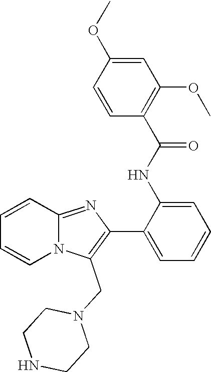 | | NA | |
| 362 | 502 | 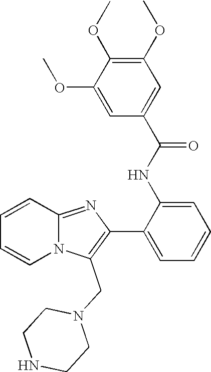 | | B | B |
| 364 | 472 | 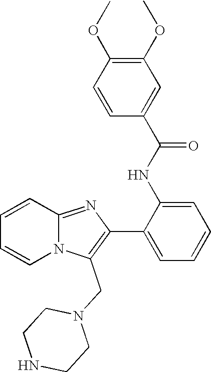 | | D | A |
| 367 | 359.1 | 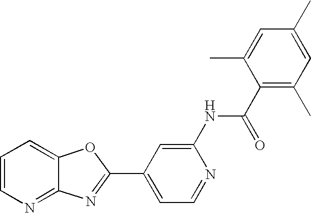 | | NA | |
| 369 | 350.8 | 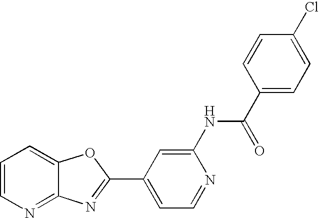 | | NA | |
| 370 | 437.0 | 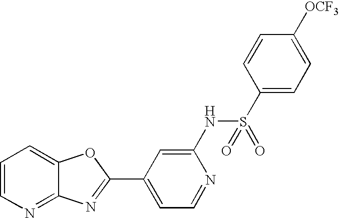 | | NA | |
| 371 | 381.1 | 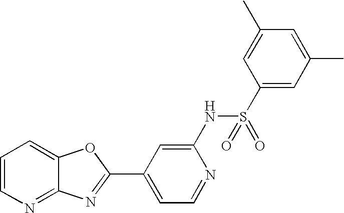 | | NA | |
| 372 | 431.1 | 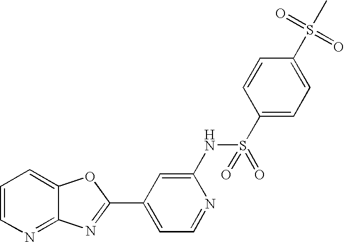 | | NA | |
| 373 | 445.0 | 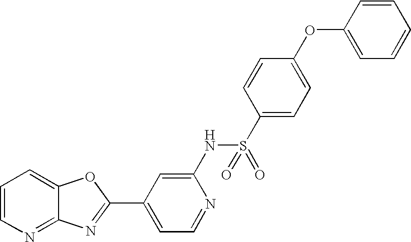 | | NA | |
| 374 | 421.1 | 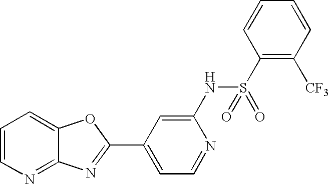 | | NA | |
| 375 | 358.2 | 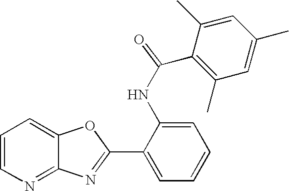 | | NA | |
| 376 | 350.0 | 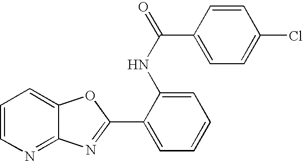 | | NA | |
| 377 | | 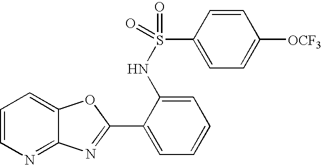 | | NA | |
| 378 | 412.1 | 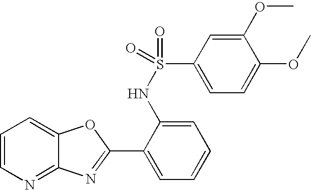 | | NA | |
| 379 | 380.0 | 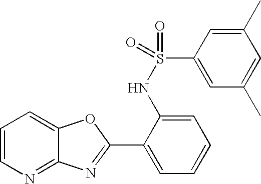 | | NA | |
| 380 | 429.9 | 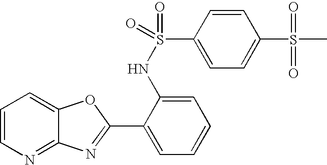 | | NA | |
| 381 | 444.1 | 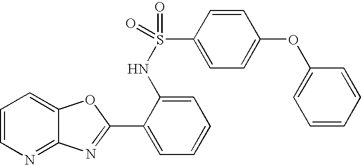 | | NA | |
| 382 | 420.0 | 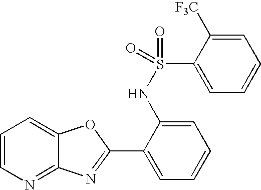 | | NA | |
| 383 | 515.7 | 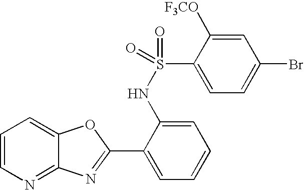 | | NA | |
| 384 | 487.8 | 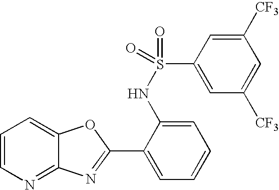 | | NA | |
| 385 | 397.2 | 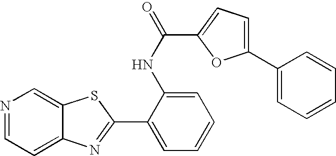 | | NA | |
| 387 | 366.8 | 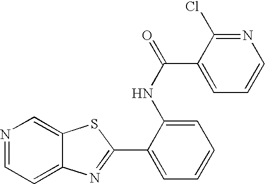 | | NA | |
| 390 | 412.5 | 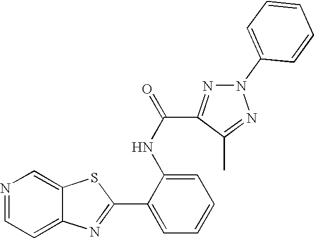 | | A | B |
| 391 | 333.4 | 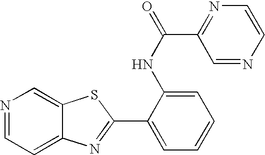 | | A | B |
| 392 | 424.5 | 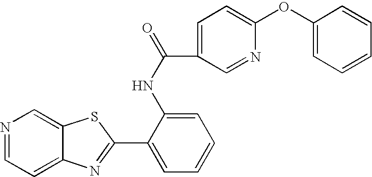 | | A | B |
| 393 | 400.3 | 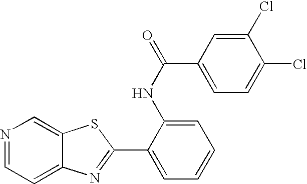 | | NA | |
| 394 | 457.6 | 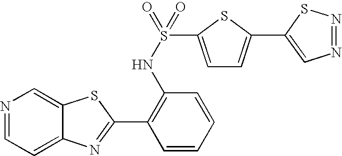 | | NA | |
| 396 | 389.5 | 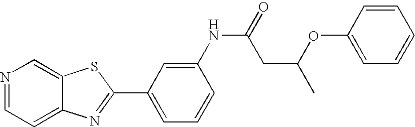 | | A | B |
| 398 | 460.1 | 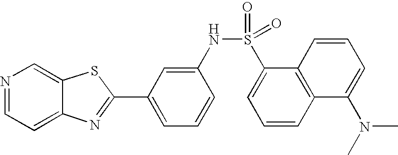 | | A | B |
| 399 | 424.5 | 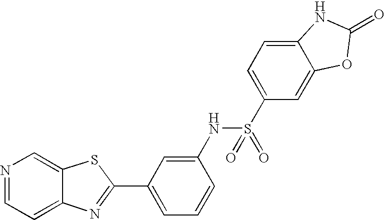 | | A | B |
| 400 | 392 | 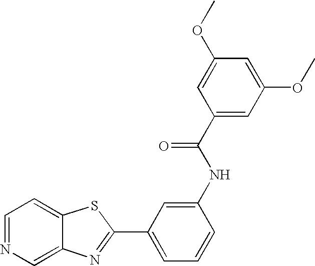 | | NA | |
| 401 | 422 | 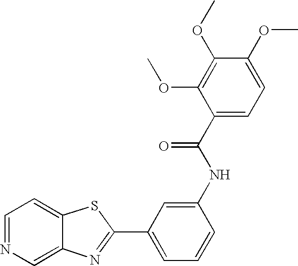 | | A | B |
| 402 | 375 | 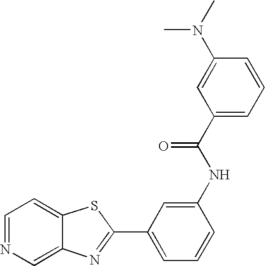 | | A′ | B |
| 403 | 375 | 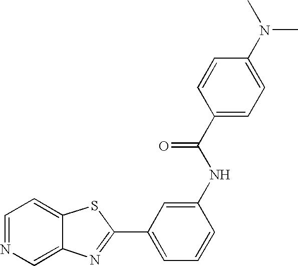 | | A′ | B |
| 404 | 376 | 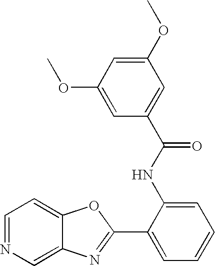 | | A′ | B |
| 405 | 406 | 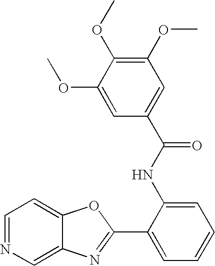 | | A′ | B |
| 406 | 359 | 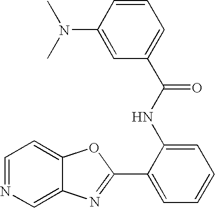 | | A′ | B |
| 407 | 359 | 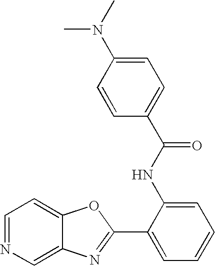 | | A′ | B |
| 408 | 359 | 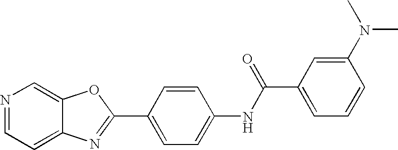 | | B | B |
| 409 | 422 | 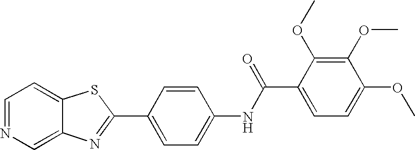 | | NA | |
| 410 | 359 | 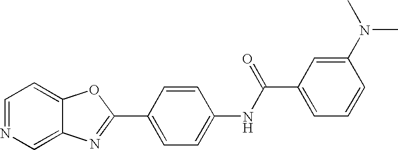 | | NA | |
| 411 | 422 | 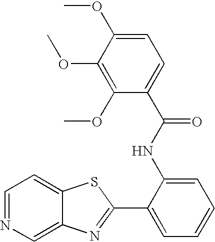 | | A | B |
| 412 | 406 | 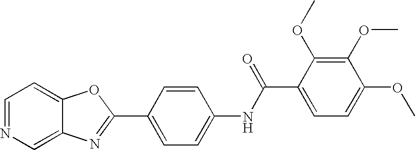 | | NA | |
| 413 | 385.1 | 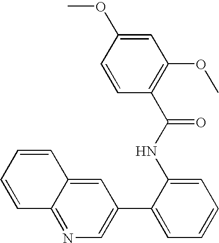 | | B | B |
| 414 | 385 | 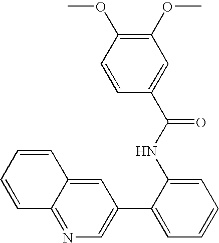 | | A | B |
| 415 | 415 | 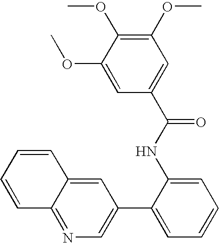 | | B | B |
| 416 | 368 | 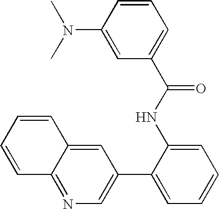 | | NA | |
| 419 | 406 | 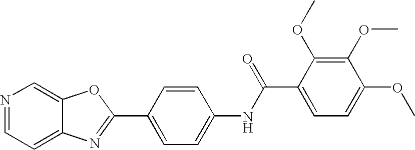 | | NA | |
| 420 | 376 | 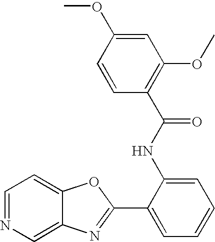 | | NA | |
| 421 | 406 | 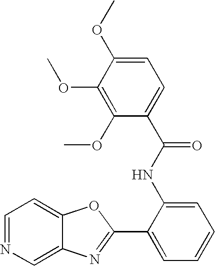 | | A | B |
| 422 | 382 | 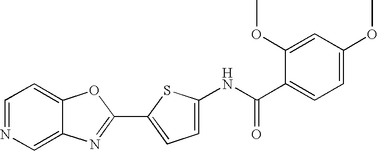 | | A | B |
| 423 | 382 | 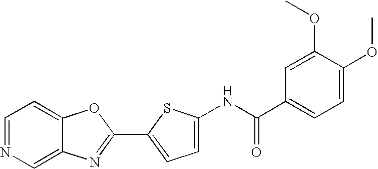 | | A′ | B |
| 424 | 382 | 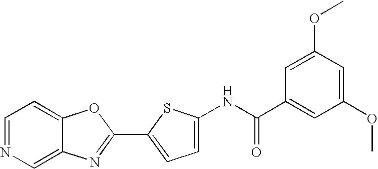 | | NA | |
| 425 | 412 | 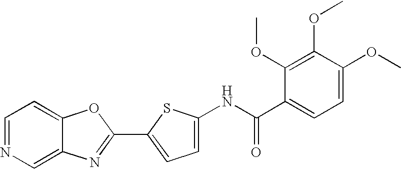 | | NA | |
| 426 | 412 | 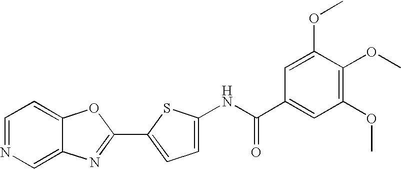 | | A′ | B |
| 427 | 365 | 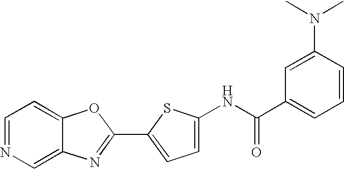 | | A′ | B |
| 428 | 365 | 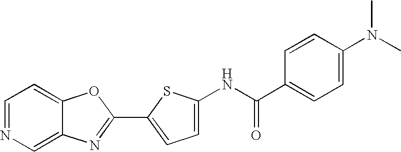 | | NA | |
| 429 | 376 | 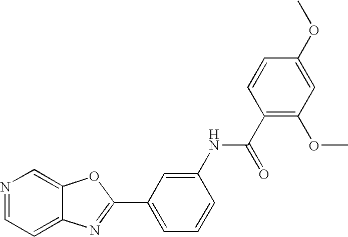 | | A′ | B |
| 430 | 406 | 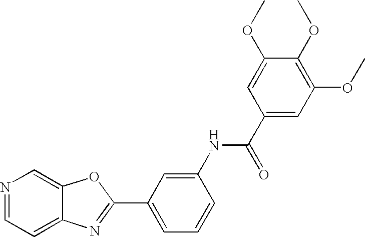 | | A | A |
| 431 | 359 | 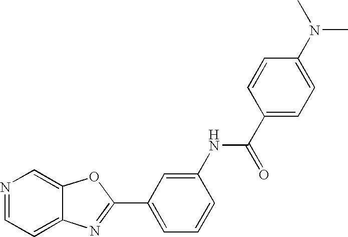 | | A | B |
| 436 | 445.1 | 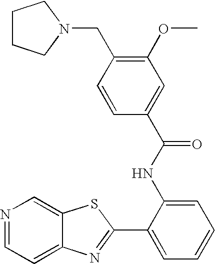 | | A | A |
| 437 | 375.1 | 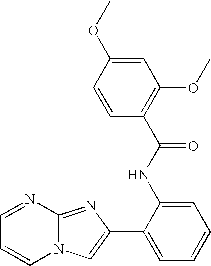 | | A | B |
| 438 | 375 | 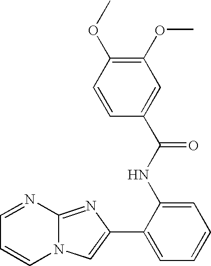 | | A′ | A |
| 439 | 405 | 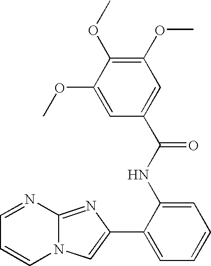 | | A′ | B |
| 440 | 468 | 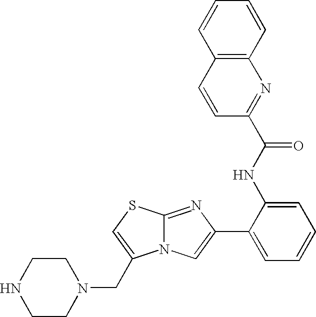 | | A′ | A |
| 441 | 470 | 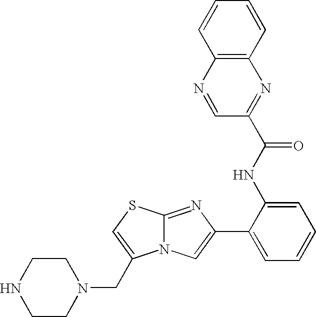 | | A′ | A |
| 442 | 472 | 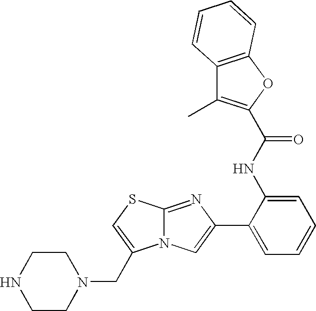 | | A′ | A |
| 443 | 436 | 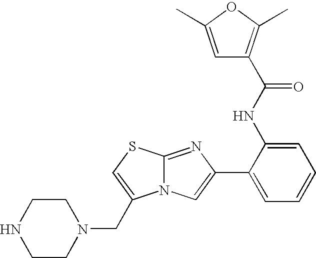 | | A | A |
| 444 | 464 | 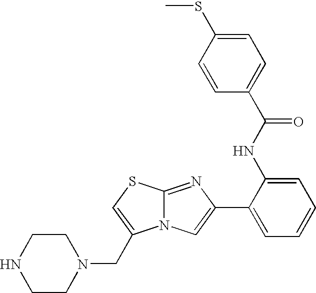 | | A | A |
| 445 | 432 | 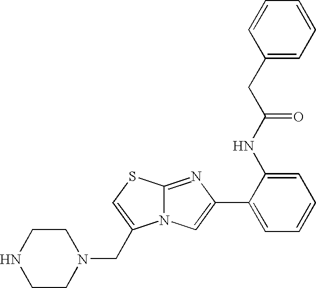 | | A | B |
| 446 | 424 | 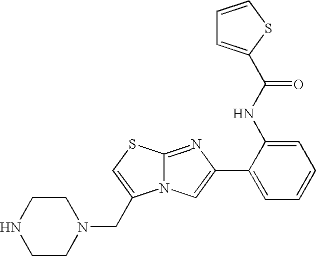 | | A | B |
| 447 | 484 | 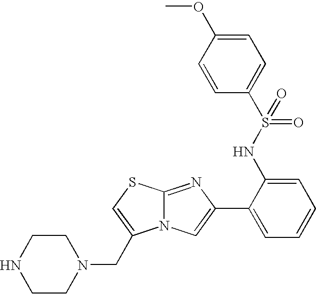 | | NA | |
| 448 | 510 | 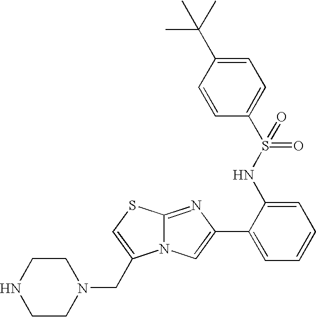 | | NA | |
| 449 | 376 | 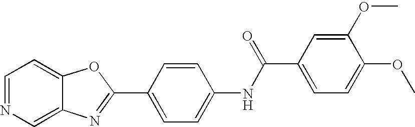 | | NA | |
| 450 | 392 | 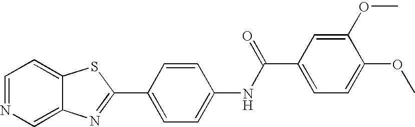 | | NA | |
| 451 | 376 | 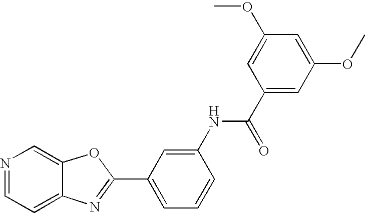 | | A | B |
| 452 | 406 | 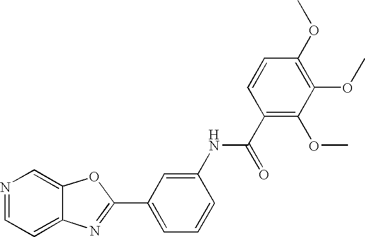 | | A | B |
| 453 | 359 | 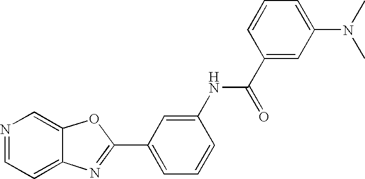 | | A | B |
| 454 | 376 | 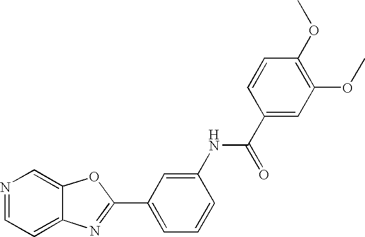 | | A | B |
| 455 | 376 | 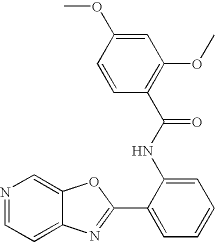 | | A′ | B |
| 456 | 376 | 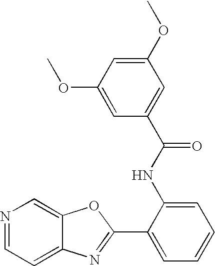 | | A′ | B |
| 457 | 406 | 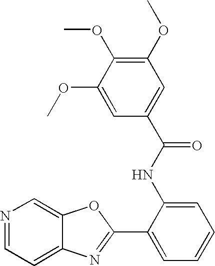 | | A | B |
| 458 | 359 | 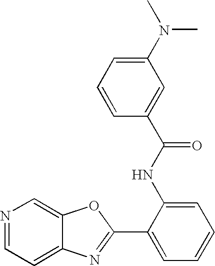 | | A′ | B |
| 459 | 359 | 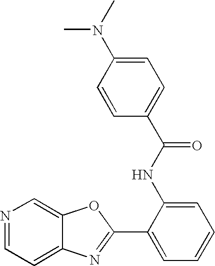 | | A′ | B |
| 460 | 359.4 | 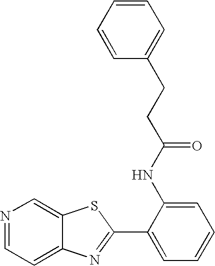 | | A | B |
| 461 | 367.4 | 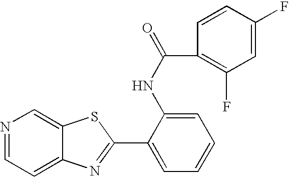 | | NA | |
| 462 | 391.5 | 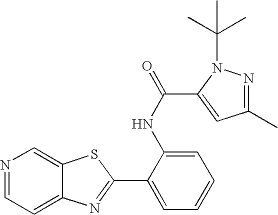 | | A′ | B |
| 463 | 375.4 | 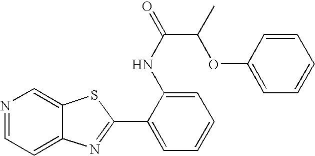 | | A | B |
| 465 | 395.9 | 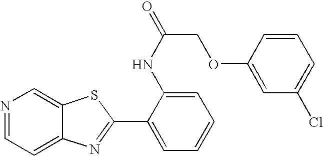 | | NA | |
| 466 | 445.5 | 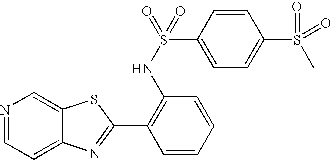 | | NA | |
| 467 | 427.2 | 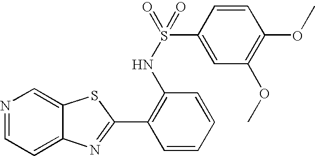 | | NA | |
| 468 | 345.0 | 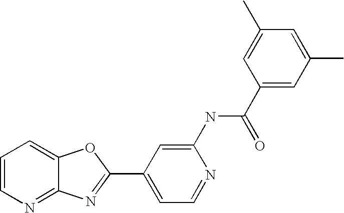 | | A | B |
| 469 | 435.4 | 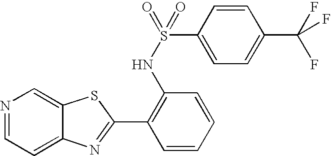 | | NA | |
| 470 | 365.0 | 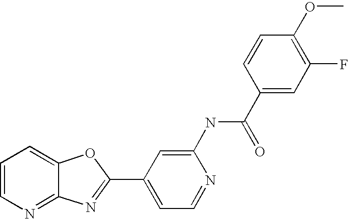 | | NA | |
| 471 | 488.9 | 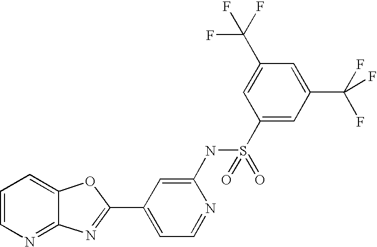 | | NA | |
| 472 | 437.5 | 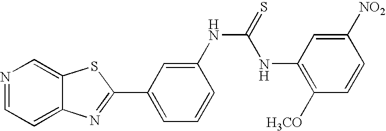 | | NA | |
| 473 | 420.5 | 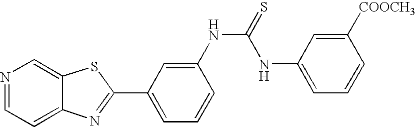 | | A′ | B |
| 474 | | 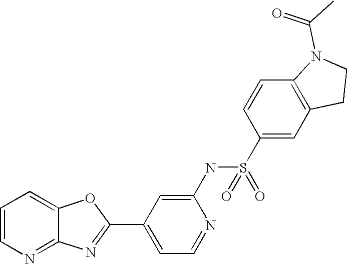 | | NA | |
| 475 | 408.6 | 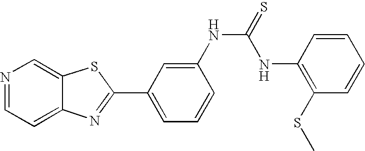 | | NA | |
| 476 | 410.4 | 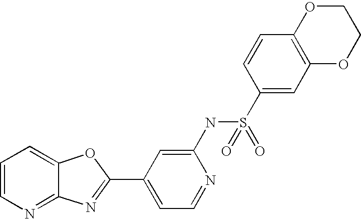 | | NA | |
| 477 | 435.9 | 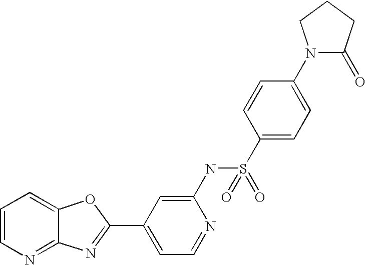 | | NA | |
| 478 | 404.8 | 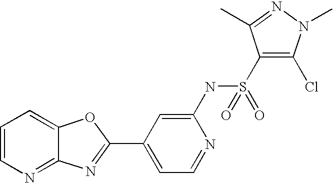 | | NA | |
| 479 | 420.9 | 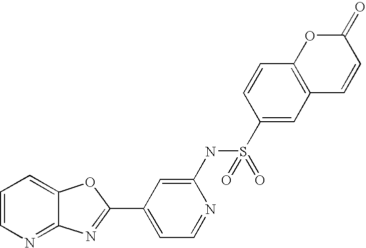 | | NA | |
| 480 | 428.5 | 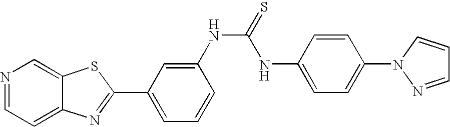 | | A | B |
| 481 | 344.1 | 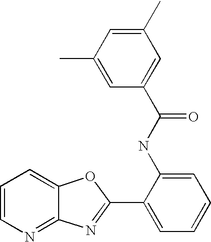 | | A′ | B |
| 483 | 364.1 | 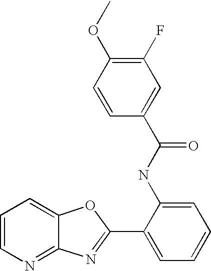 | | NA | |
| 484 | 448.6 | 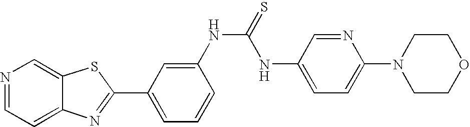 | | A | B |
| 485 | 363.5 | 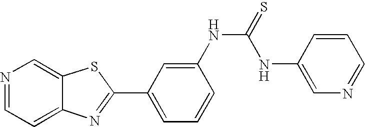 | | A | A |
| 486 | 385.9 | 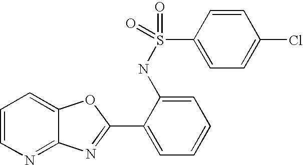 | | NA | |
| 487 | 396.1 | 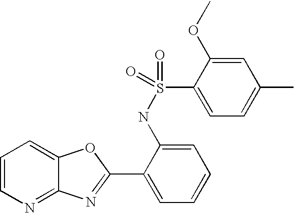 | | NA | |
| 488 | 435.1 | 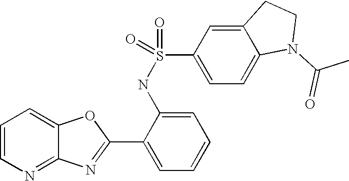 | | NA | |
| 489 | 410.1 | 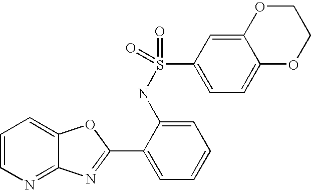 | | NA | |
| 490 | 434.9 | 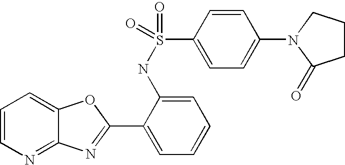 | | NA | |
| 491 | 420.5 | 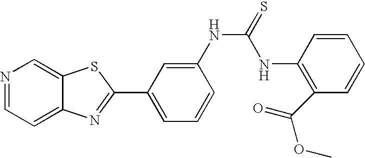 | | NA | |
| 492 | 404.0 | 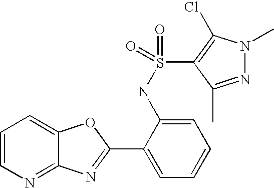 | | NA | |
| 493 | | 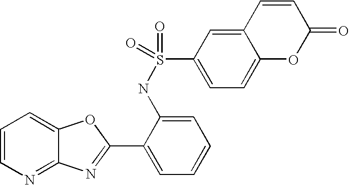 | | NA | |
| 494 | 422.9 | 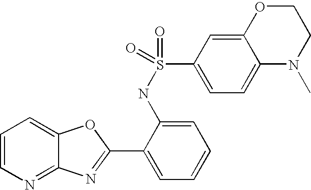 | | NA | |
| 495 | 366.0 | 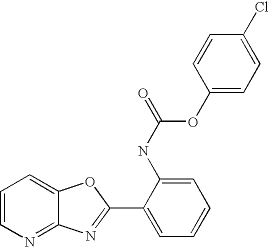 | | NA | |
| 496 | 349.9 | 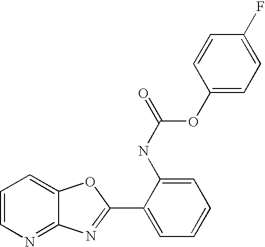 | | NA | |
| 497 | 346.0 | 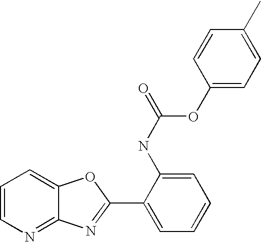 | | NA | |
| 498 | 406.5 | 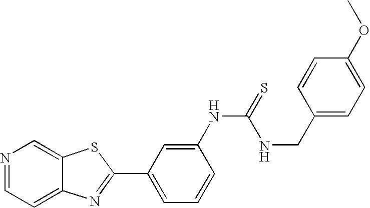 | | A | B |
| 499 | 362.1 | 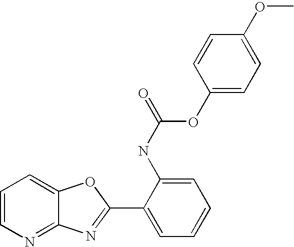 | | NA | |
| 500 | | 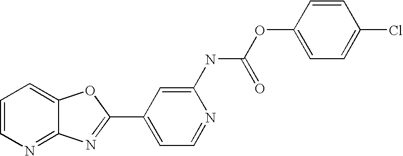 | | NA | |
| 501 | 347.1 | 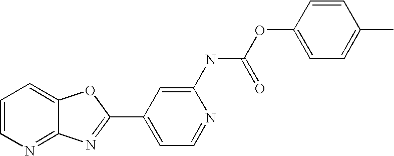 | | NA | |
| 502 | 363.1 | 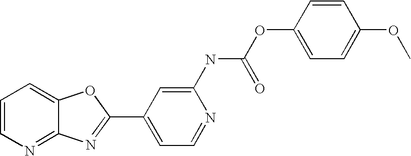 | | NA | |
| 503 | 443.1 | 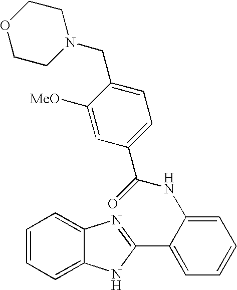 | | A′ | A |
| 504 | 358 | 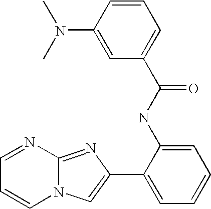 | | A | B |
| 505 | 359 | 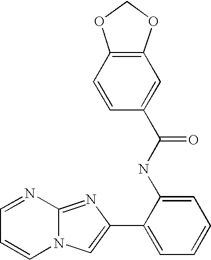 | | NA | |
| 506 | 429.1 | 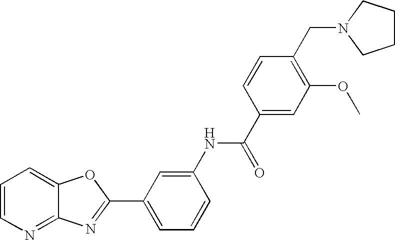 | | A | A |
| 507 | 388.1 | 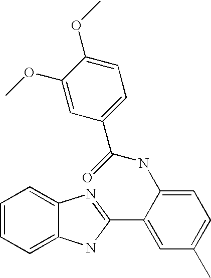 | | A′ | A |
| 508 | 366.1 | 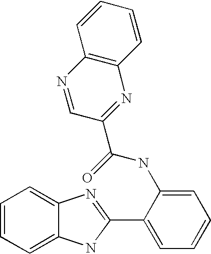 | | A′ | A |
| 510 | 457 | 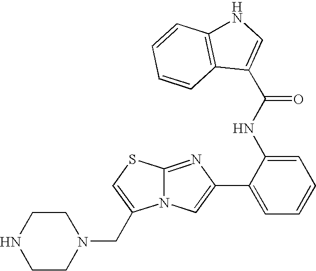 | | A′ | A |
| 511 | 460 | 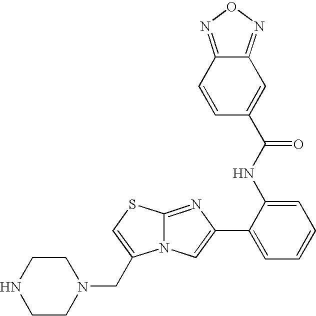 | | NA | |
| 512 | 484 | 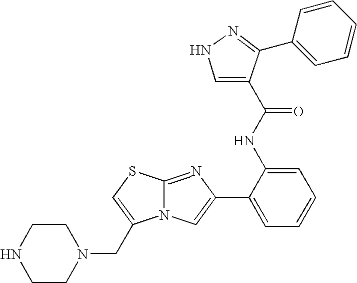 | | A′ | A |
| 513 | 470 | 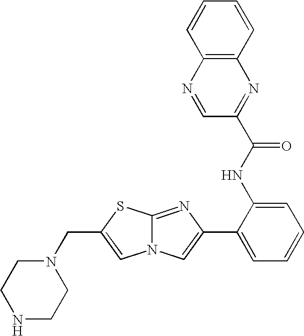 | | A′ | A |
| 514 | 466 | 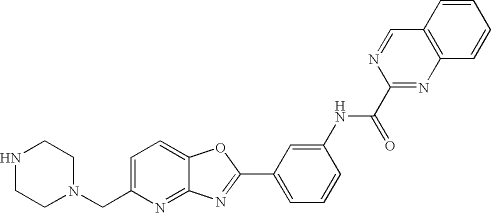 | | A | B |
| 515 | 398 | 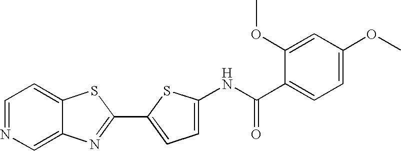 | | A′ | B |
| 516 | 398 | 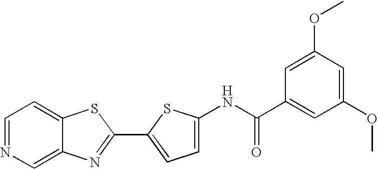 | | NA | |
| 517 | 428 | 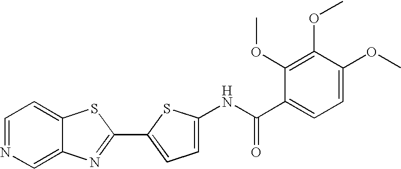 | | NA | |
| 518 | 381 | 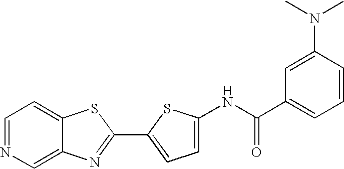 | | A′ | B |
| 519 | 381 | 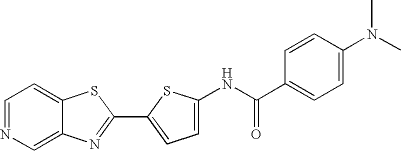 | | A′ | B |
| 520 | 428 | 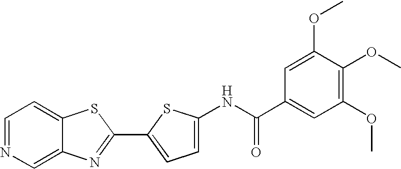 | | A′ | B |
| 521 | 375.0 | 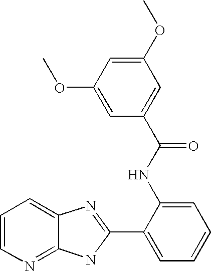 | | A′ | B |
| 522 | 405.0 | 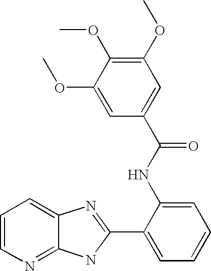 | | A | A |
| 523 | 359.0 | 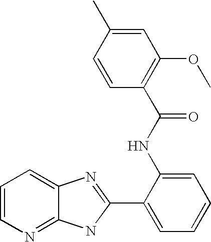 | | A | B |
| 524 | 358.0 | 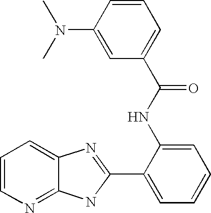 | | A′ | B |
| 525 | 375.0 | 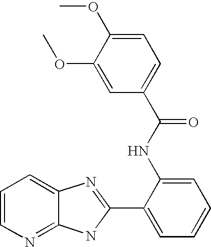 | | A′ | B |
| 526 | 400.0 | 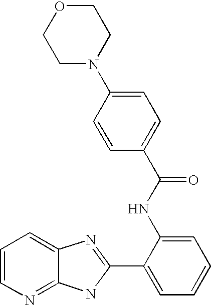 | | A′ | A |
| 527 | 398.0 | 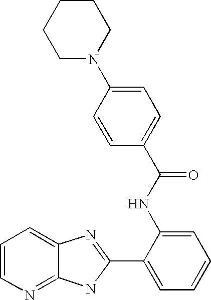 | | A′ | B |
| 528 | 412.9 | 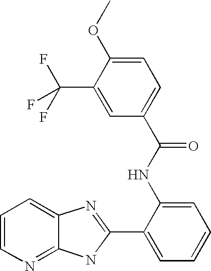 | | A′ | B |
| 529 | 399.1 | 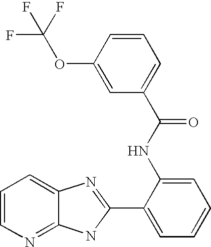 | | A′ | B |
| 530 | 359.0 | 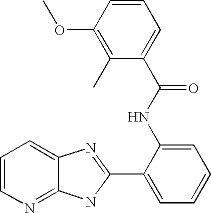 | | A′ | B |
| 531 | 345.0 | 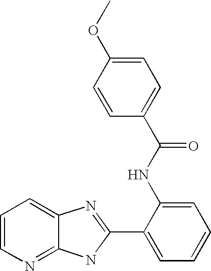 | | A′ | B |
| 532 | 345.0 | 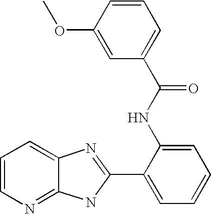 | | A′ | B |
| 533 | 315.0 | 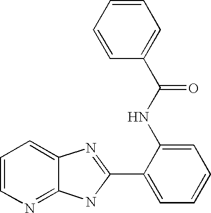 | | A′ | B |
| 534 | 358.0 | 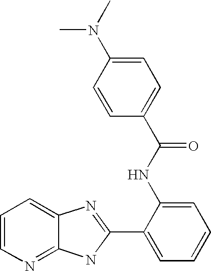 | | A′ | B |
| 535 | 428.1 | 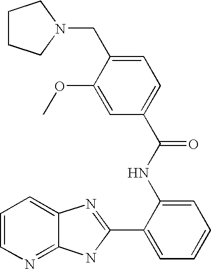 | | A | A |
| 536 | 442.1 | 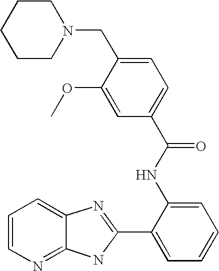 | | A | A |
| 537 | 444.0 | 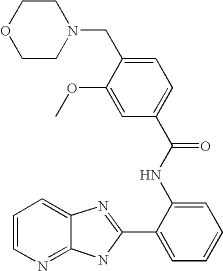 | | A′ | A |
| 538 | 443.1 | 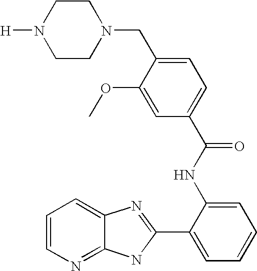 | | A | A |
| 539 | 457.1 | 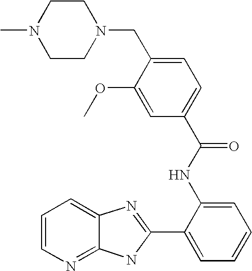 | | A′ | A |
| 540 | 402.1 | 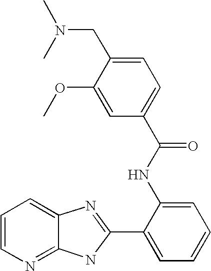 | | A | A |
| 541 | 443.1 | 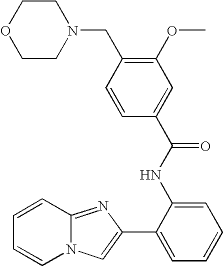 | | A | B |
| 542 | 444 | 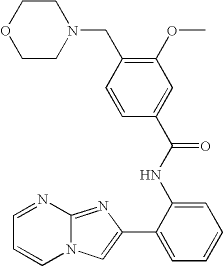 | | A | A |
| 543 | 420 | 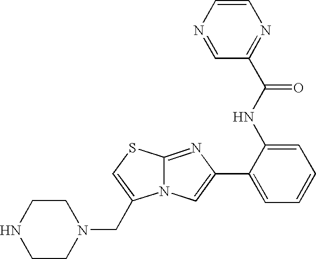 | | A′ | A |
| 544 | 474 | 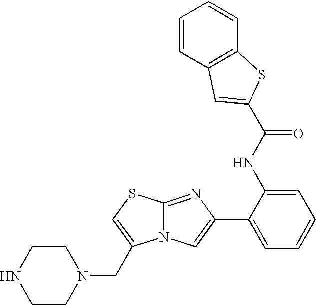 | | A | A |
| 545 | 378.1 | 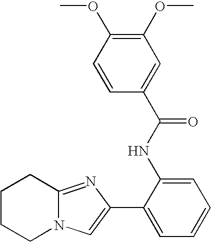 | | A | B |
| 546 | 408 | 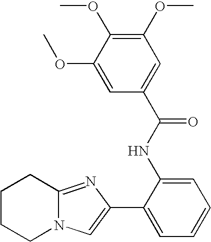 | | A | B |
| 547 | 369 | 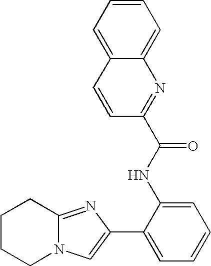 | | A′ | B |
| 548 | 370 | 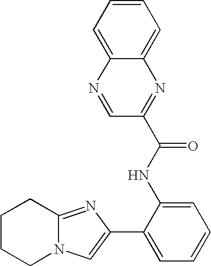 | | A′ | B |
| 556 | 365.1 | 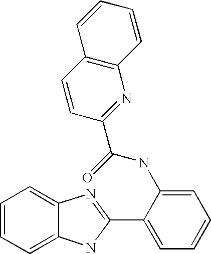 | | A′ | A |
| 557 | 542.1 | 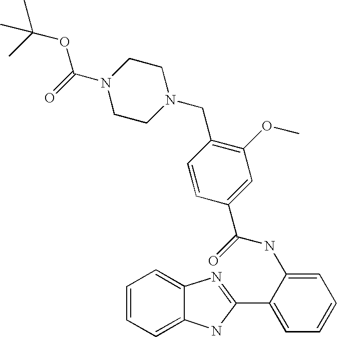 | | NA | |
| 558 | 442.1 | 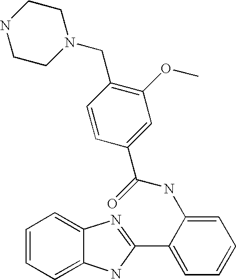 | | A′ | A |
| 559 | 508 | 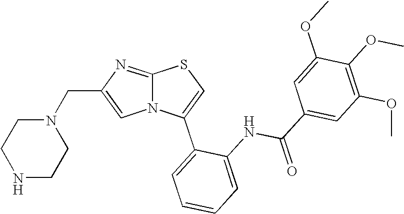 | | NA | |
| 560 | 470 | 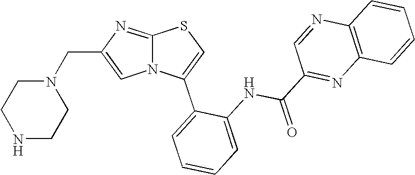 | | NA | |
| 561 | 382 | 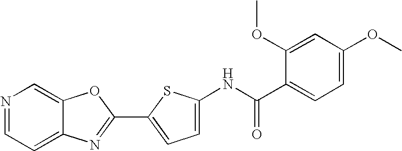 | | A′ | B |
| 562 | 382 | 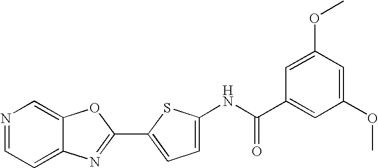 | | A′ | B |
| 563 | 412 | 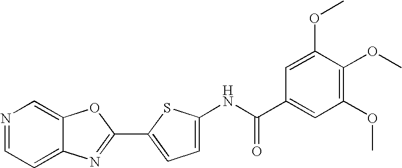 | | A | B |
| 565 | 400.8 | 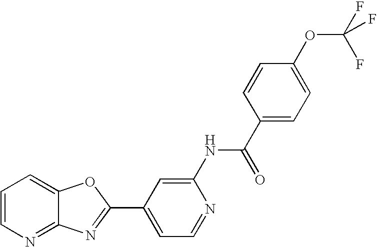 | | NA | |
| 566 | 409.9 | 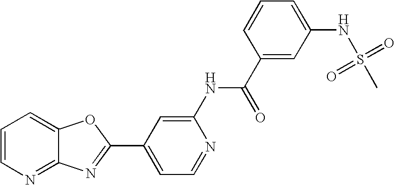 | | A | B |
| 567 | 374.9 | 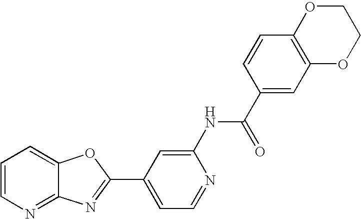 | | NA | |
| 568 | 367.0 | 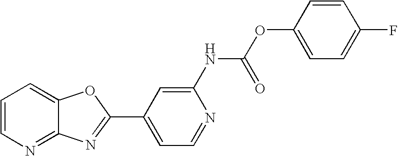 | | NA | |
| 569 | 374.9 | 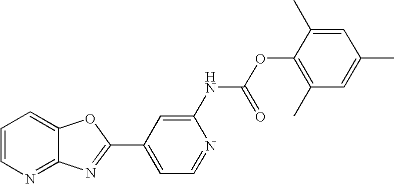 | | NA | |
| 570 | 361.0 | 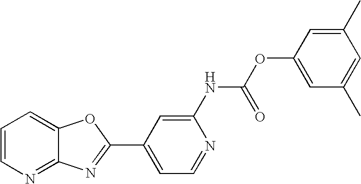 | | NA | |
| 571 | 444.0 | 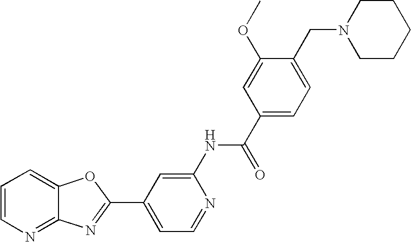 | | A | B |
| 572 | 429.9 | 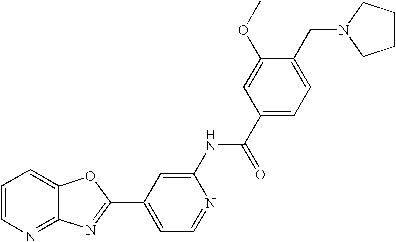 | | B | B |
| 573 | 431.8 | 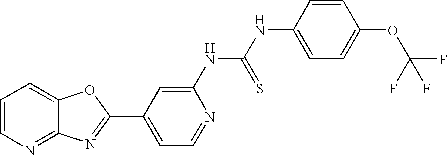 | | NA | |
| 574 | 376.2 | 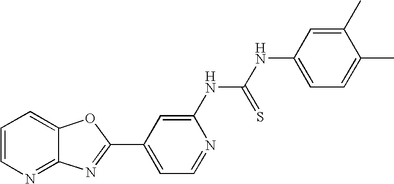 | | NA | |
| 575 | 439.9 | 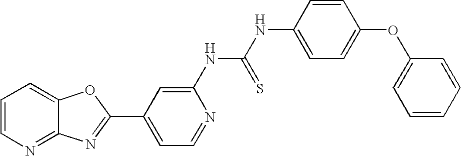 | | NA | |
| 576 | 376.1 | 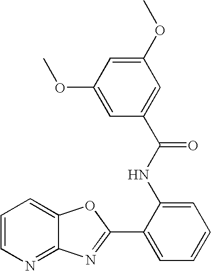 | | NA | |
| 577 | 400.1 | 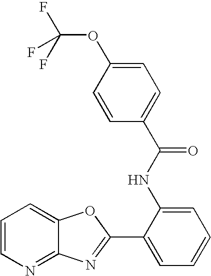 | | NA | |
| 578 | 368.1 | 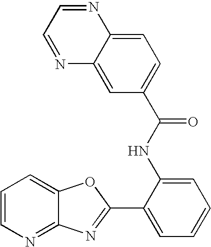 | | NA | |
| 579 | 401.1 | 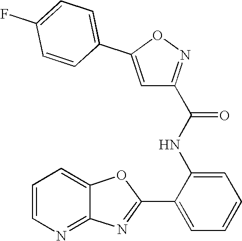 | | NA | |
| 580 | 374.0 | 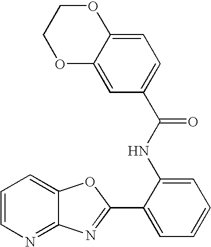 | | NA | |
| 581 | 334.0 | 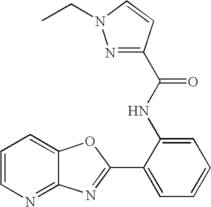 | | A | B |
| 582 | 400.0 | 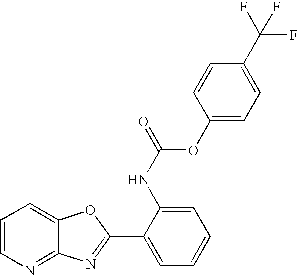 | | NA | |
| 583 | 374.1 | 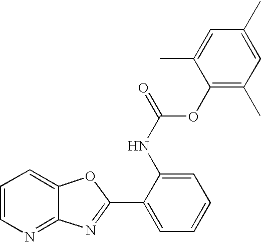 | | NA | |
| 584 | 360.0 | 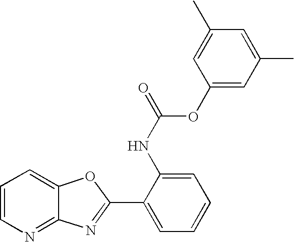 | | NA | |
| 585 | 443.1 | 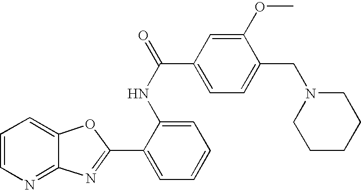 | | A | B |
| 587 | 427.1 | 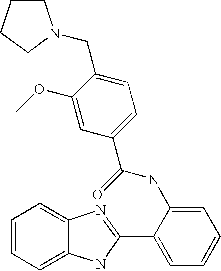 | | A′ | B |
| 588 | 520 | 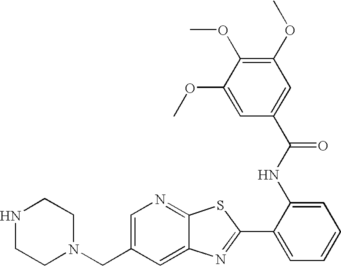 | | A | A |
| 589 | 490 | 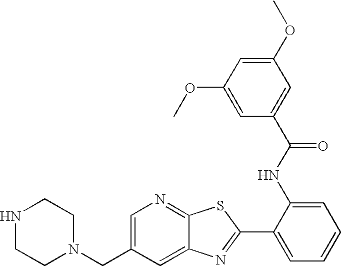 | | A′ | A |
| 590 | 474 | 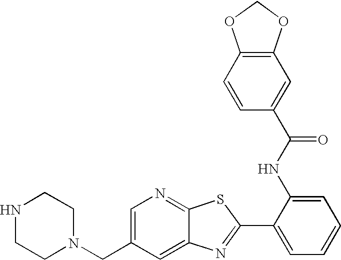 | | A | B |
| 591 | 458 | 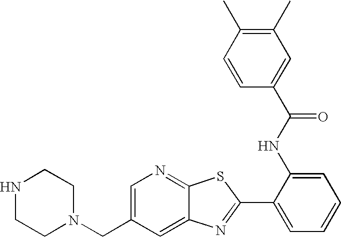 | | A′ | A |
| 592 | 455 | 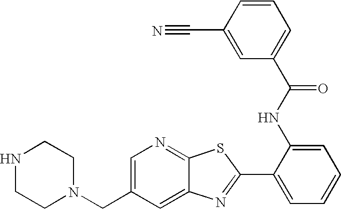 | | A′ | B |
| 593 | 473 | 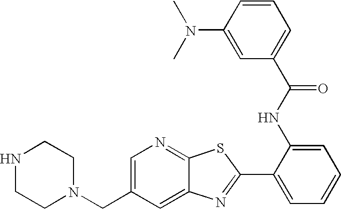 | | A′ | A |
| 594 | 466 | 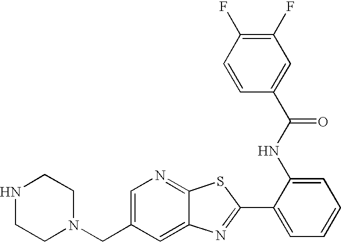 | | A | B |
| 596 | 467.4 | 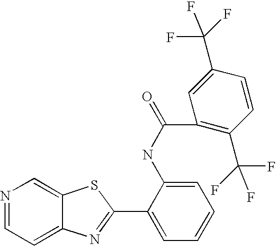 | | A′ | B |
| 597 | 377.2 | 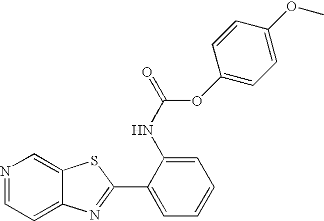 | | A′ | B |
| 598 | 422.3 | 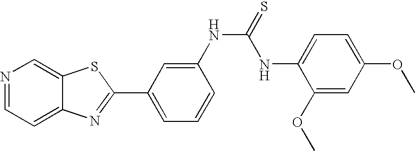 | | A′ | B |
| 600 | 422.5 | 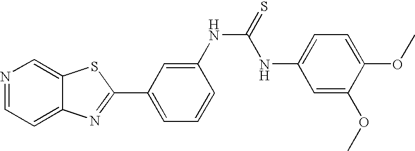 | | D | B |
| 601 | 405.5 | 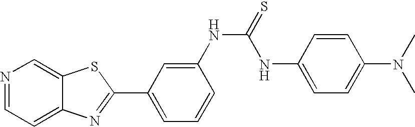 | | NA | |
| 604 | 360.4 | 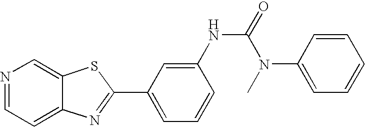 | | NA | |
| 605 | 377.4 | 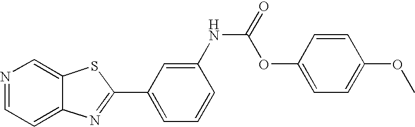 | | NA | |
| 607 | 415.4 | 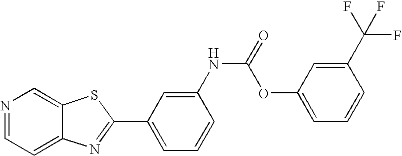 | | NA | |
| 608 | 332.4 | 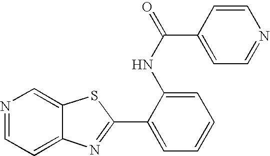 | | NA | |
| 609 | 439.3 | 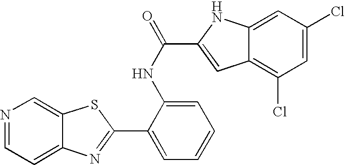 | | NA | |
| 610 | 418.6 | 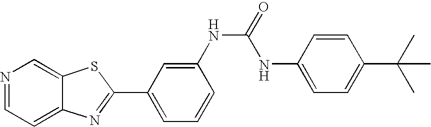 | | NA | |
| 611 | 465.6 | 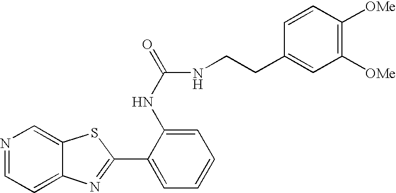 | | A′ | B |
| 612 | 402.5 | 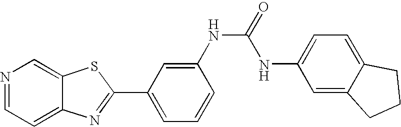 | | NA | |
| 614 | 428.5 | 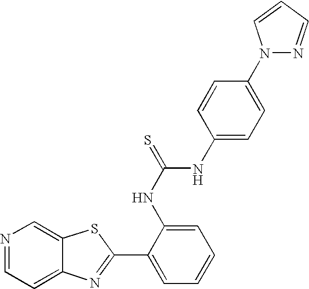 | | NA | |
| 615 | 408.6 | 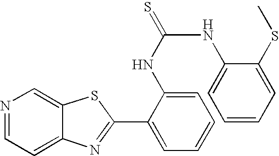 | | NA | |
| 616 | 437.5 | 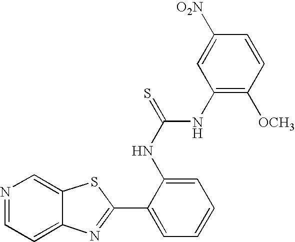 | | | |
| 617 | 471.1 | 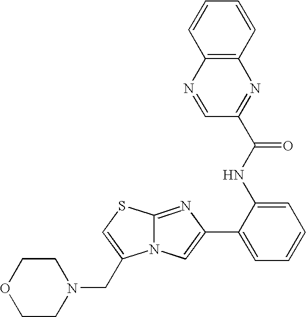 | | A′ | A |
| 618 | 469.1 | 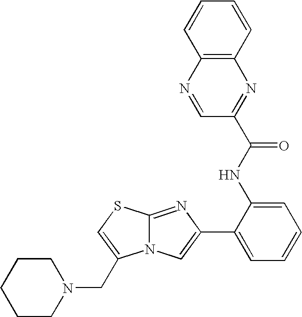 | | A′ | B |
| 619 | 455 | 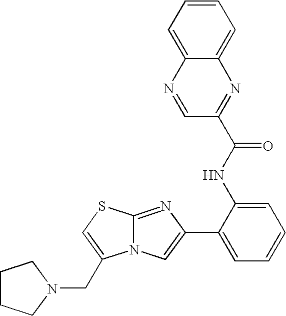 | | A′ | B |
| 620 | 493.1 | 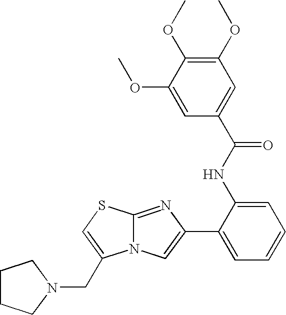 | | A′ | B |
| 621 | 472 | 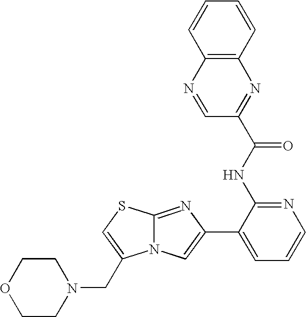 | | A′ | A |
| 622 | 482 | 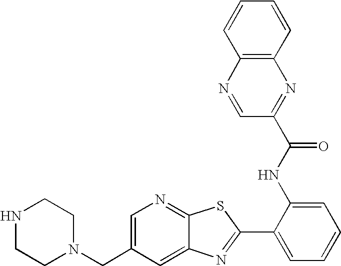 | | A′ | A |
| 623 | 437 | 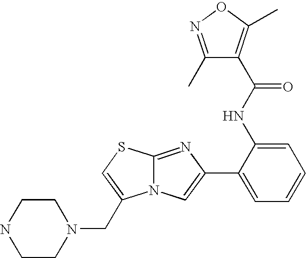 | | NA | C |
| 624 | 458 | 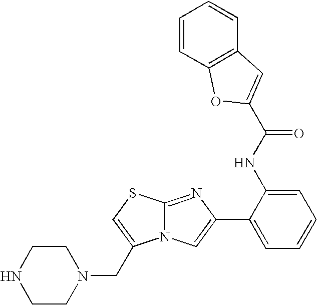 | | A′ | A |
| 625 | 496 | 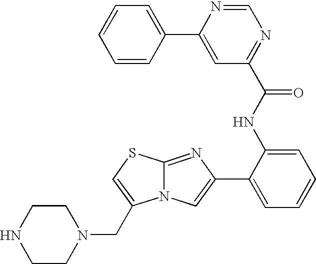 | | A′ | A |
| 628 | 574.1 | 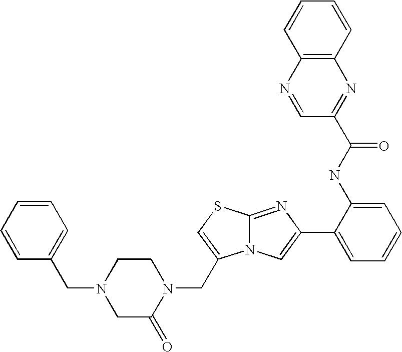 | | A′ | B |
| 629 | 429.0 | 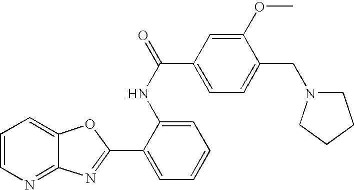 | | A | A |
| 630 | 403.1 | 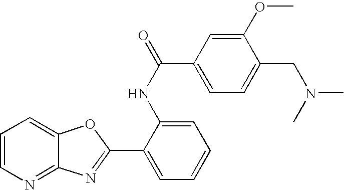 | | A | A |
| 631 | 445.0 | 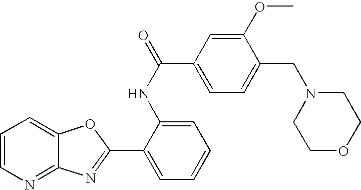 | | A′ | B |
| 632 | 458.1 | 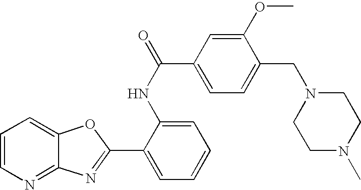 | | A | A |
| 633 | 423.3 | 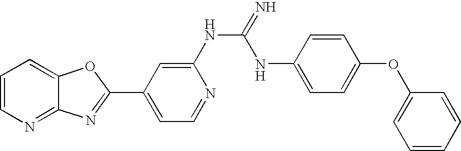 | | NA | C |
| 634 | 364.9 | 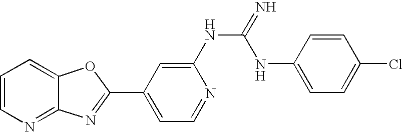 | | D | B |
| 635 | 421.1 | 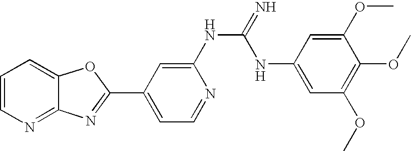 | | D | A |
| 636 | 359.9 | 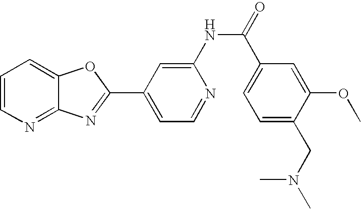 | | B | B |
| 637 | 459.3 | 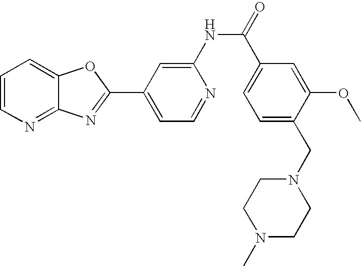 | | D | B |
| 638 | 445.2 | 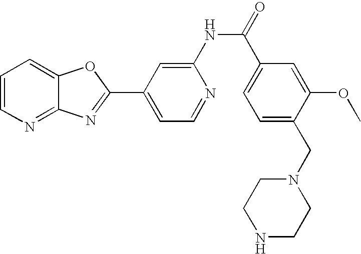 | | D | B |
| 639 | 378.9 | 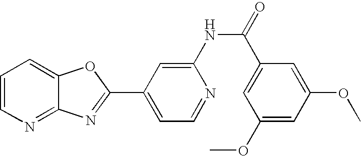 | | A | B |
| 640 | 368.9 | 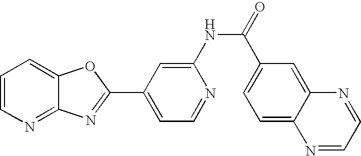 | | NA | C |
| 641 | 334.9 | 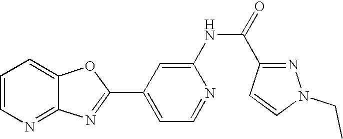 | | D | B |
| 642 | 446.2 | 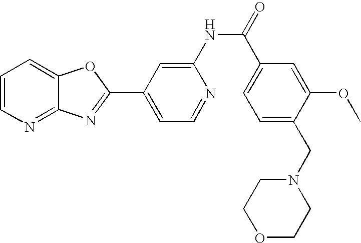 | | D | B |
| 643 | 535.1 | 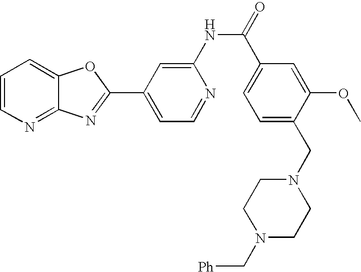 | | A | B |
| 644 | 434 | 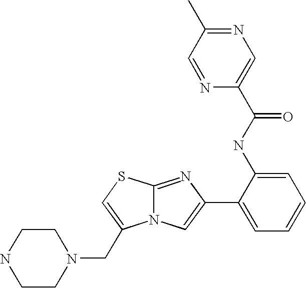 | | A | A |
| 645 | 469 | 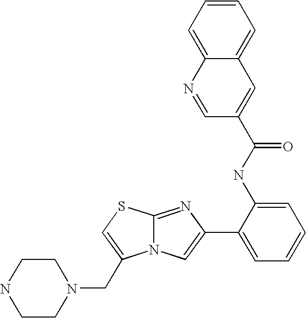 | | A′ | A |
| 646 | 481 | 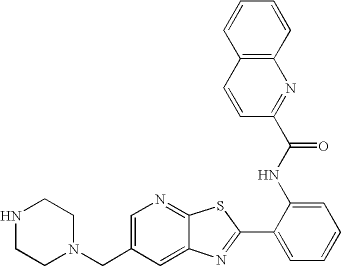 | | A | A |
| 647 | 473.1 | 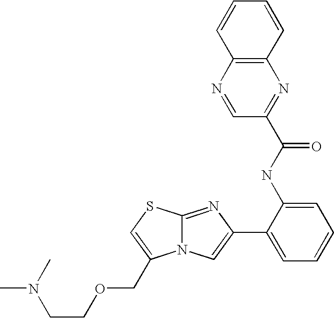 | | A′ | A |
| 648 | 402.1 | 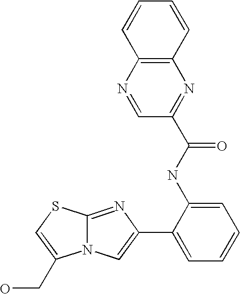 | | A′ | B |
| 649 | 512.2 | 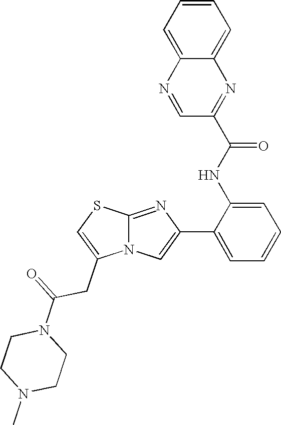 | | A′ | A |
| 650 | 570.2 | 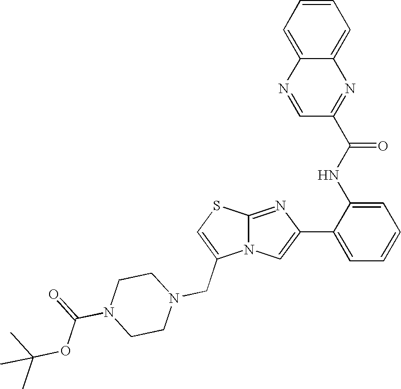 | | NA | |
| 651 | 512.2 | 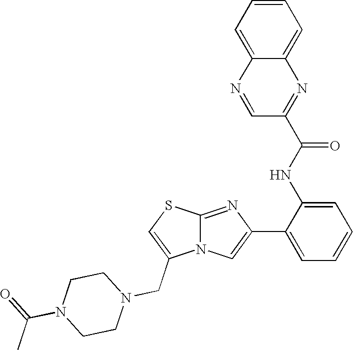 | | A′ | A |
| 655 | 393.0 | 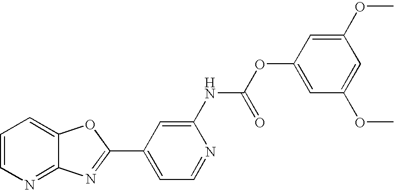 | | NA | C |
| 656 | 393.2 | 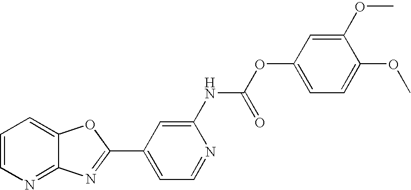 | | NA | C |
| 657 | 423.1 | 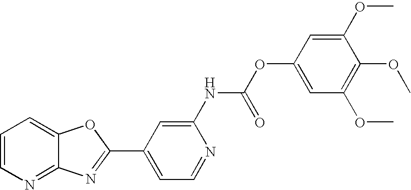 | | NA | C |
| 658 | 382.1 | 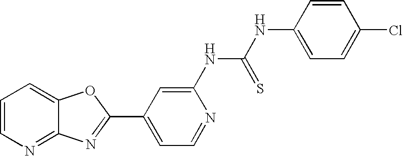 | | NA | C |
| 659 | 509.2 | 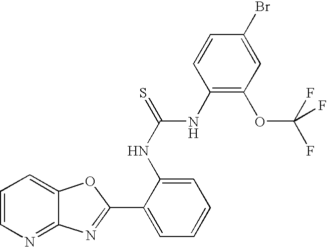 | | NA | C |
| 660 | 408.1 | 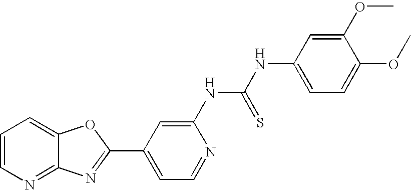 | | A | B |
| 661 | 408.2 | 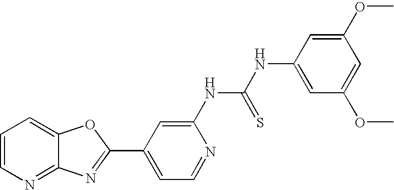 | | A′ | B |
| 662 | 378.2 | 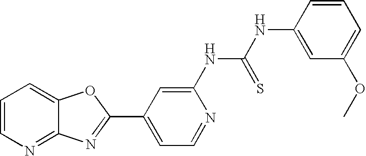 | | NA | C |
| 663 | 438.0 | 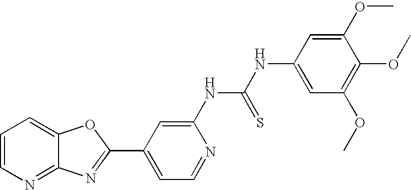 | | A | B |
| 664 | 477.8 | 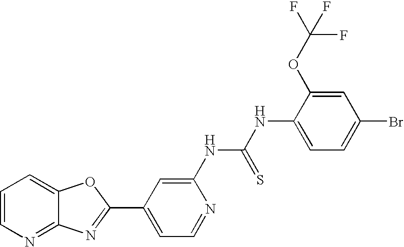 | | NA | C |
| 665 | 406.1 | 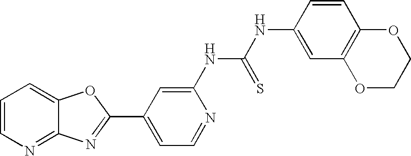 | | NA | C |
| 666 | 391.0 | 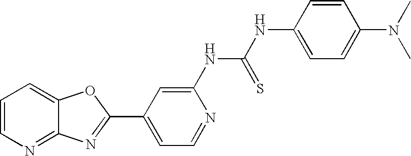 | | NA | C |
| 667 | 448.0 | 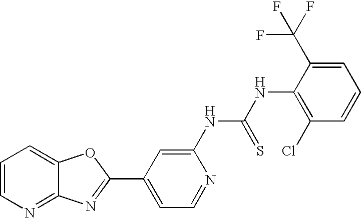 | | A | A |
| 668 | 410.0 | 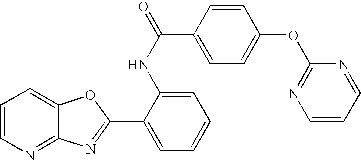 | | A | B |
| 669 | 392.0 | 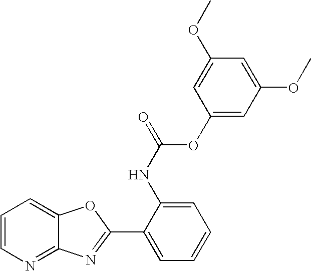 | | NA | C |
| 670 | 392.0 | 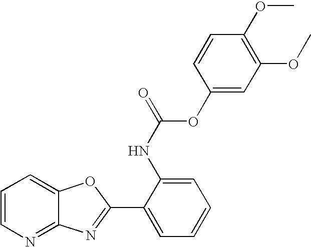 | | A | B |
| 671 | 422.0 | 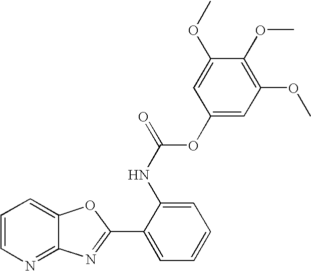 | | NA | C |
| 672 | 415.1 | 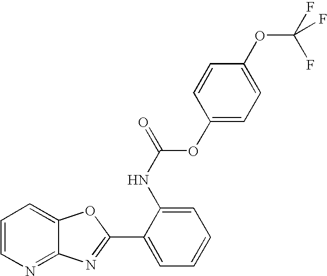 | | NA | C |
| 673 | | 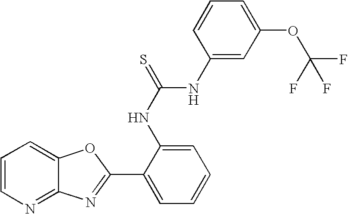 | | NA | C |
| 674 | | 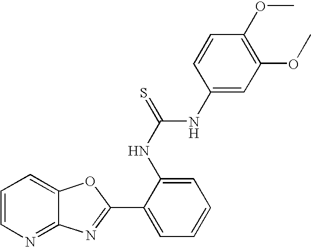 | | NA | C |
| 675 | 407.1 | 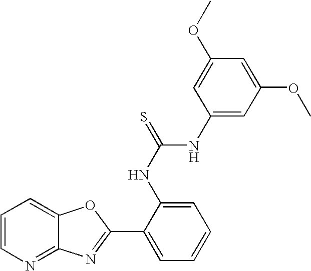 | | NA | C |
| 676 | | 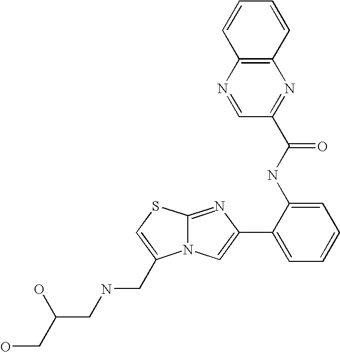 | | A′ | A |
| 677 | | 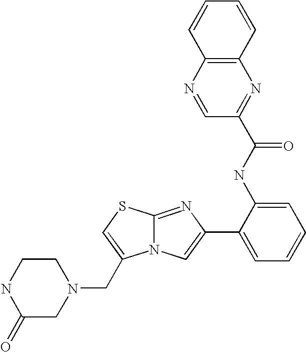 | | A′ | A |
| 678 | | 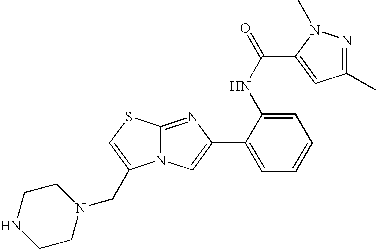 | | A | B |
| 679 | | 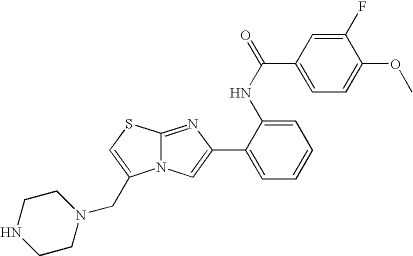 | | A | B |
| 680 | 484.2 | 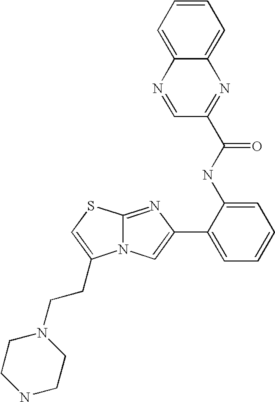 | | A′ | A |
| 681 | 522 | 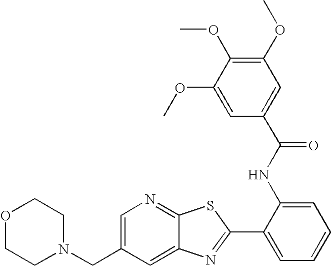 | | A | A |
| 682 | 492 | 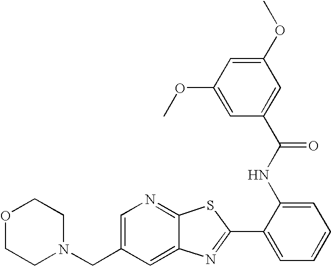 | | A′ | A |
| 683 | 475 | 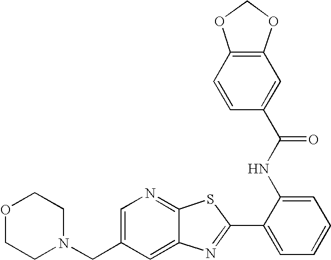 | | A | B |
| 684 | 460 | 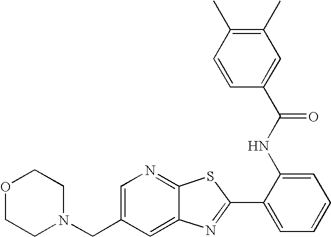 | | A | B |
| 685 | 456 | 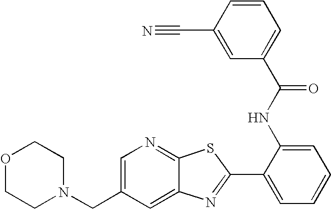 | | A′ | B |
| 686 | 475 | 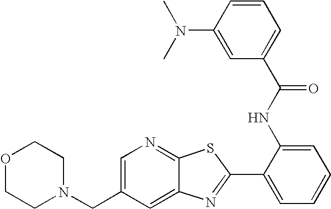 | | A′ | A |
| 687 | 467 | 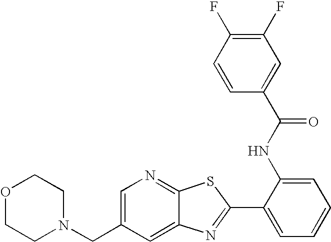 | | NA | C |
| 688 | 483 | 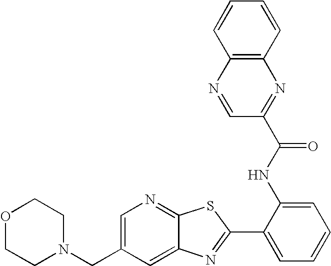 | | NA | C |
| 689 | 479 | 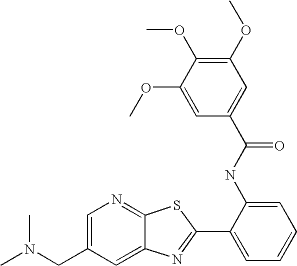 | | A | A |
| 690 | 483 | 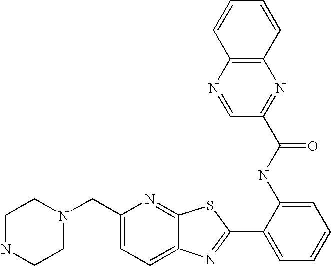 | | A′ | A |
| 692 | 469 | 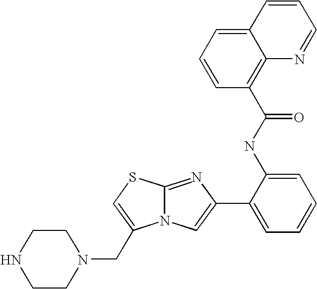 | | C | B |
| 695 | 454 | 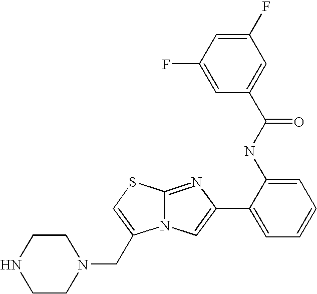 | | A′ | A |
| 697 | 596 | 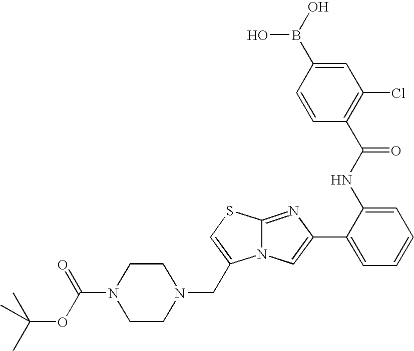 | | NA | C |
| 698 | 502 | 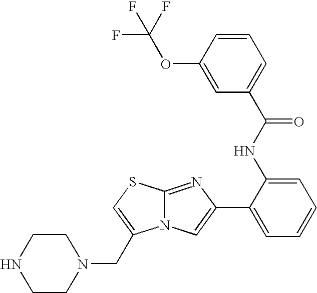 | | A′ | A |
| 699 | | 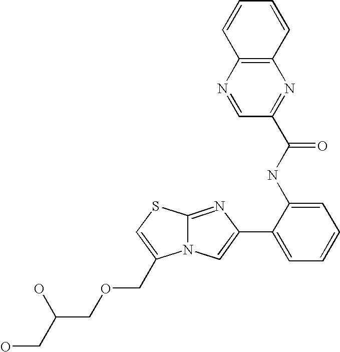 | | A | A |
| 700 | 512.2 | 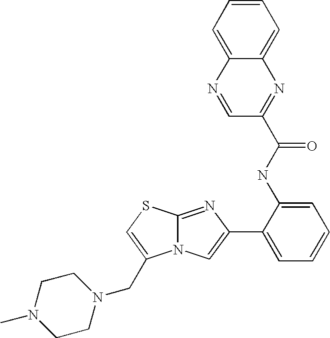 | | A′ | A |
| 701 | 477 | 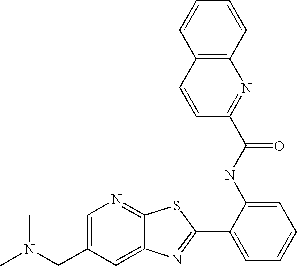 | | A′ | A |
| 702 | 534 | 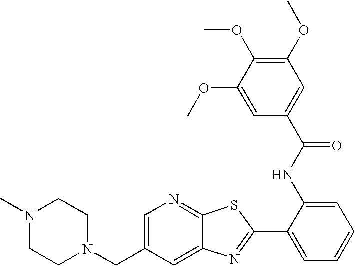 | | A | A |
| 703 | 468 | 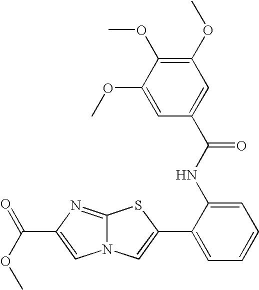 | | NA | C |
| 704 | 454 | 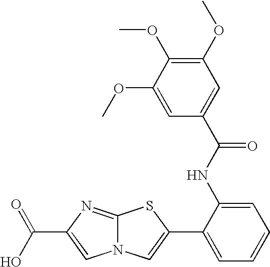 | | NA | C |
| 705 | 387 | 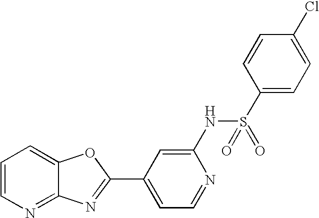 | | | |
| 706 | 445.1 | 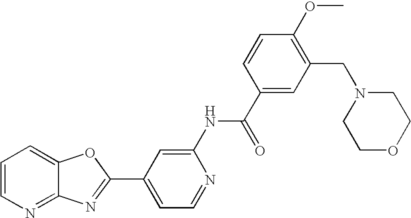 | | | |
| 707 | 386.1 | 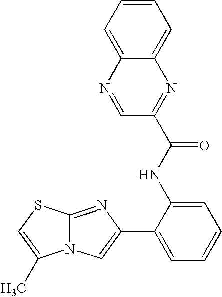 | | A′ | A |
| 708 | 494.2 | 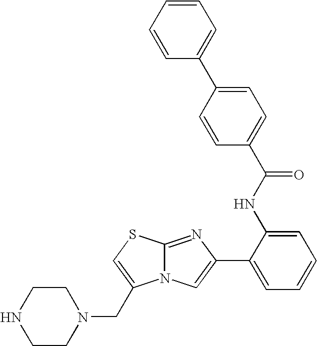 | | A′ | A |
| 709 | 494.1 | 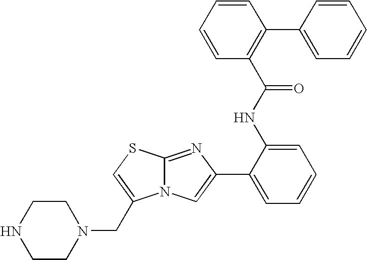 | | NA | C |
| 710 | 494.1 | 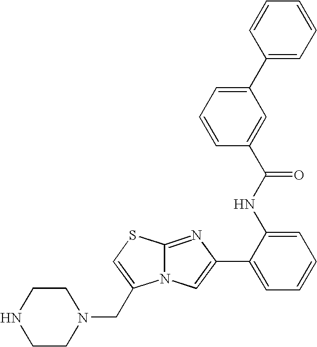 | | A′ | A |
| 711 | | 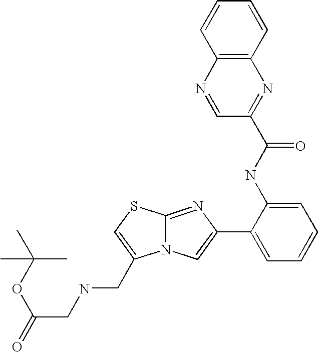 | | A | B |
| 714 | | 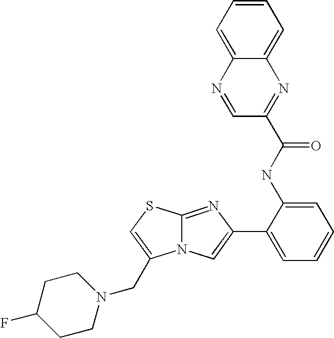 | | A | B |
| 715 | | 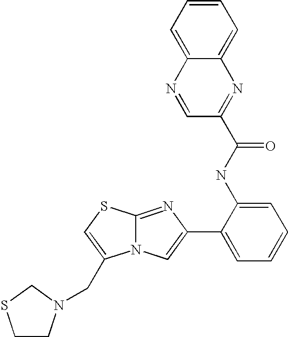 | | A′ | B |
| 716 | | 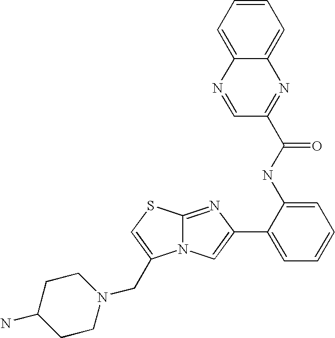 | | A′ | A |
| 717 | | 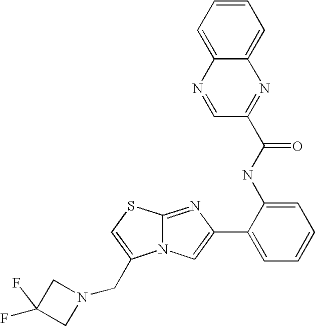 | | A | B |
| 718 | 444.1 | 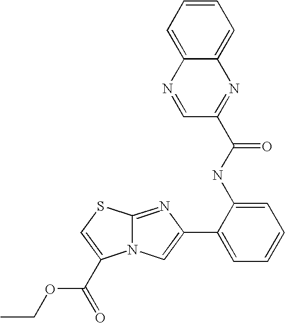 | | NA | C |
| 719 | 416 | 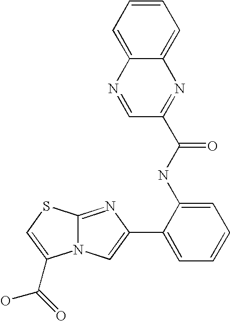 | | A | A |
| 720 | | 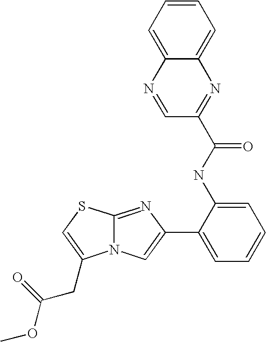 | | NA | C |
| 721 | | 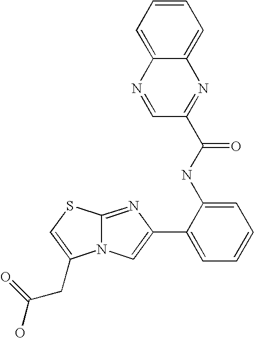 | | A | A |
| 722 | 449 | 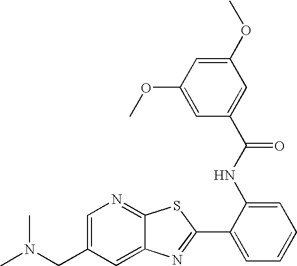 | | A′ | A |
| 723 | 490 | 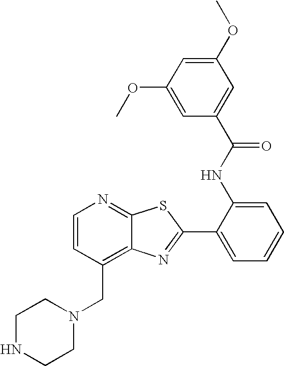 | | A | A |
| 724 | 482 | 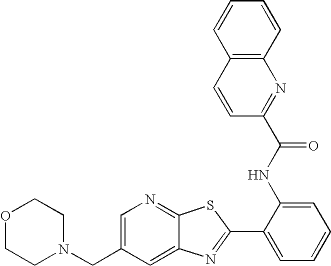 | | A′ | A |
| 725 | 505 | 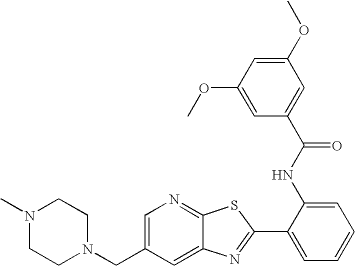 | | A′ | A |
| 726 | 491 | 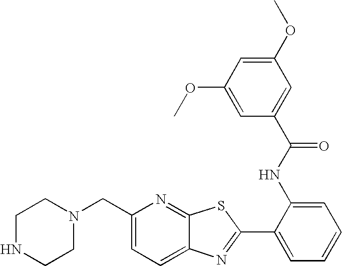 | | A′ | A |
| 727 | 458 | 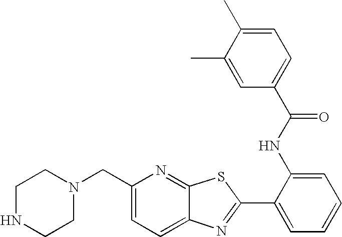 | | A′ | A |
| 728 | 466 | 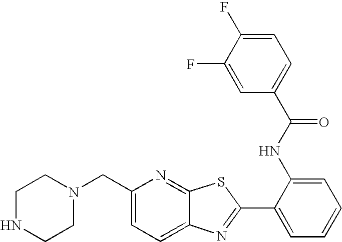 | | A | A |
| 729 | 481 | 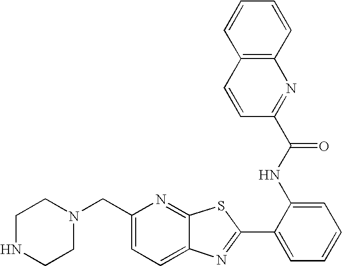 | | A′ | A |
| 730 | 488 | 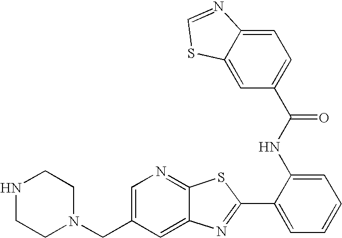 | | A | A |
| 731 | 498 | 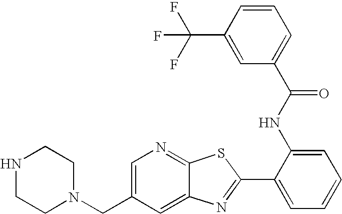 | | A | A |
| 732 | 497 | 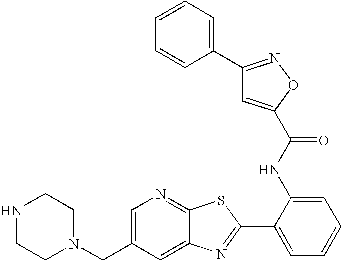 | | A′ | B |
| 733 | 484.2 | 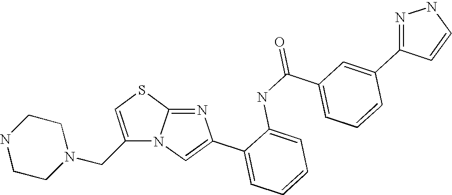 | | A′ | A |
| 735 | 514.2 | 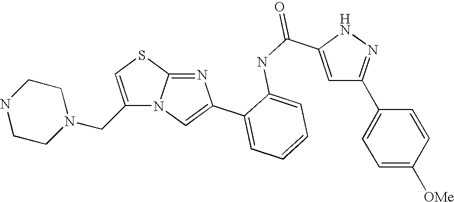 | | A′ | A |
| 736 | 514.2 | 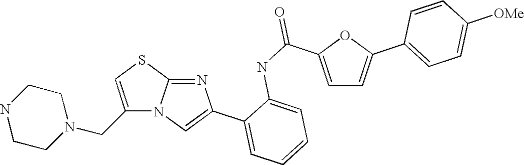 | | A′ | A |
| 737 | 500.1 | 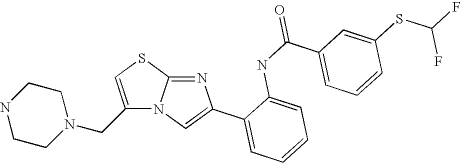 | | A′ | A |
| 738 | 448.1 | 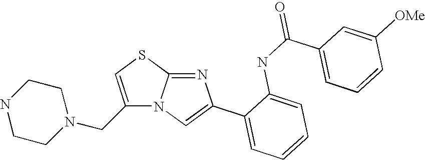 | | A | A |
| 739 | 424.1 | 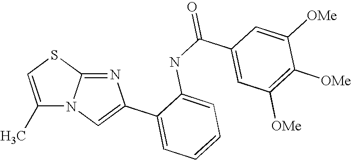 | | A′ | A |
| 740 | 377.1 | 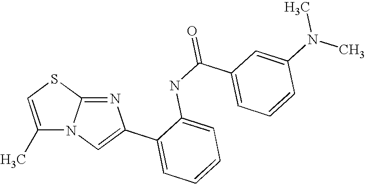 | | A′ | B |
| 741 | 498.1 | 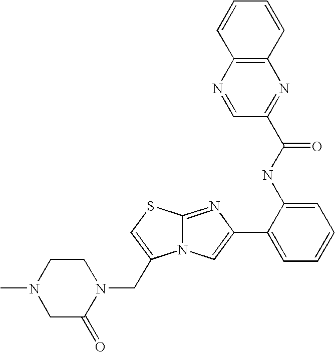 | | A′ | A |
| 742 | 487.1 | 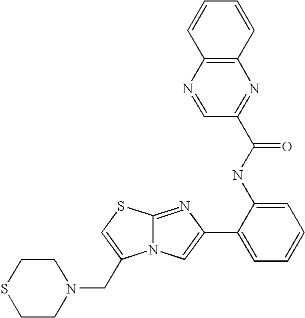 | | A′ | B |
| 743 | 466.1 | 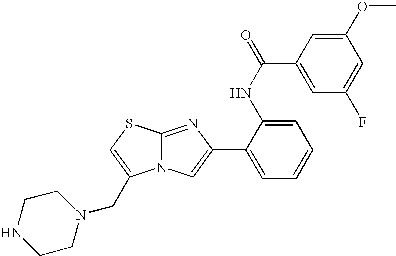 | | A | A |
| 744 | 437.1 | 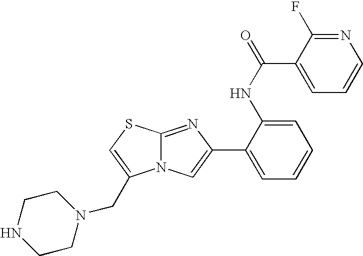 | | B | A |
| 745 | 452.1 | 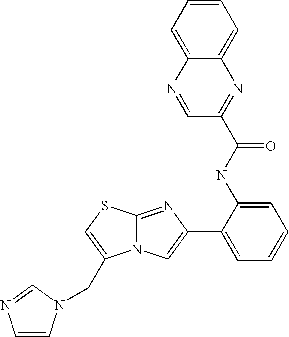 | | A′ | A |

TABLE 5

| COM- | | | | |
|---|---|---|---|---|
| POUND | | | IC₅₀ FP | IC₅₀ MS |
| NO | [M + H]+ | STRUCTURE | ASSAY | ASSAY |
| 13 | | 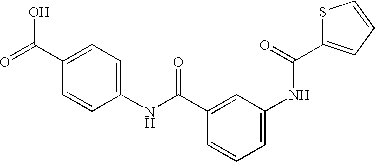 | A | |
| 14 | | 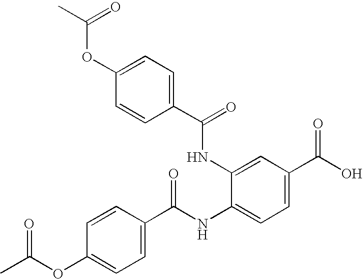 | B | |
| 15 | | 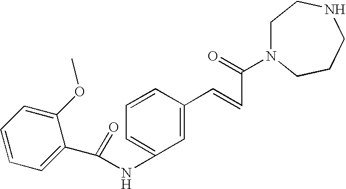 | C | |
| 23 | 359.4 | 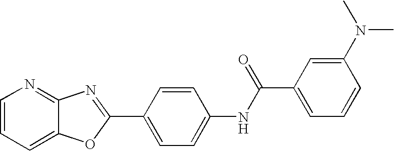 | D | |
| 25 | 341.3 | 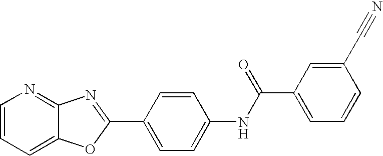 | B | |
| 26 | 341.4 | 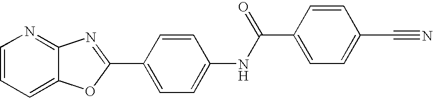 | B | |
| 28 | 401.1 | 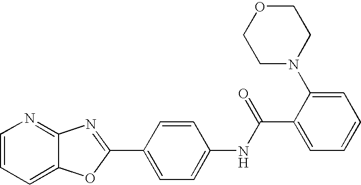 | D | |
| 30 | 380.0 | 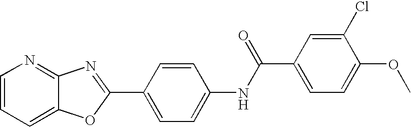 | D | |
| 44 | 341.0 | 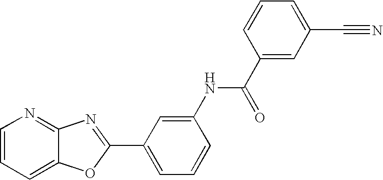 | D | |

TABLE 6

| COMPOUND NO | STRUCTURE | ED₅₀ |
|---|---|---|
| 11 | 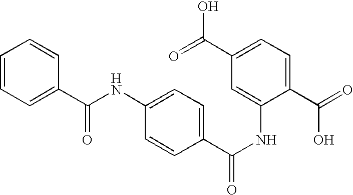 | N/A |
| 12 | 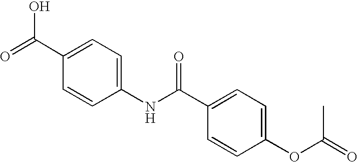 | N/A |

TABLE 8

| COMPOUND | | ED₅₀ ATP | IC₅₀ ATP |
|---|---|---|---|
| NO | STRUCTURE | ASSAY | ASSAY |
| 52 | 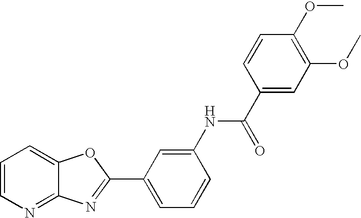 | A | |
| 42 | 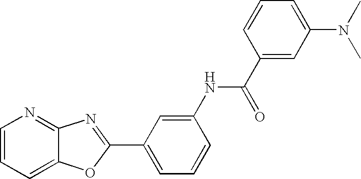 | A | |
| 49 | 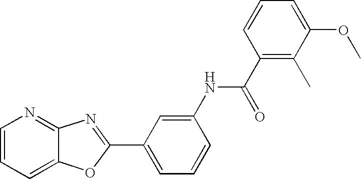 | A | |
| 115 | 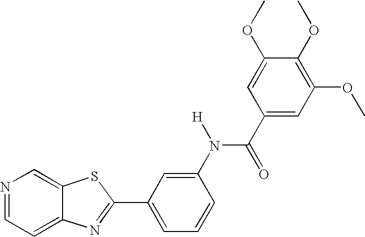 | D | |
| 79 | 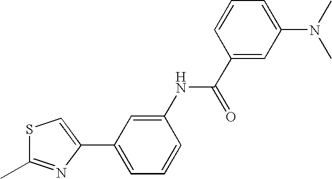 | A | |
| 117 | 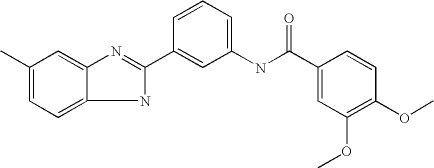 | | B |
| 120 | 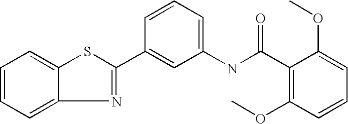 | A | |
| 121 | 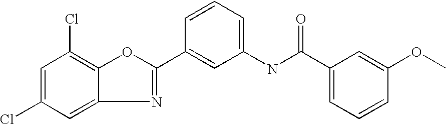 | NA | |
| 123 | 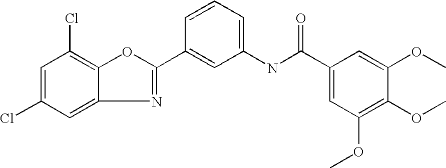 | D | |
| 85 | 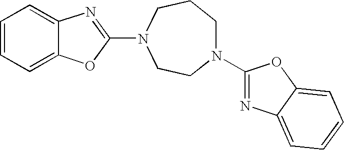 | NA | |
| 86 | 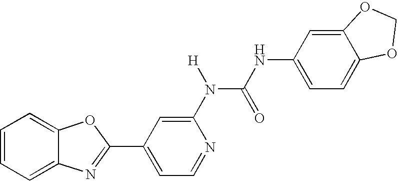 | A | |
| 87 | 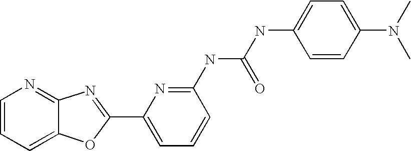 | NA | |
| 88 | 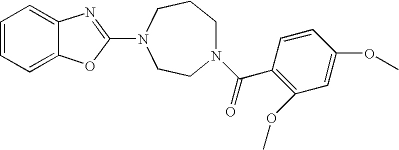 | NA | |
| 89 | 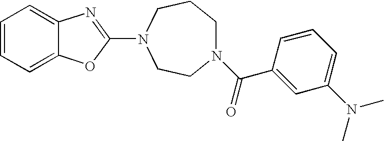 | | A |
| 90 | 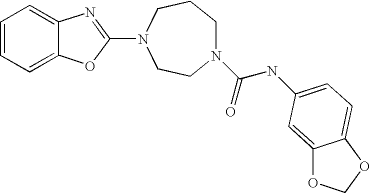 | D | |
| 91 | 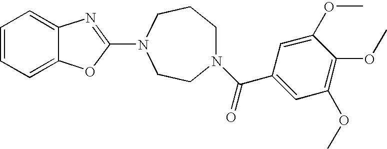 | | A |
| 92 | 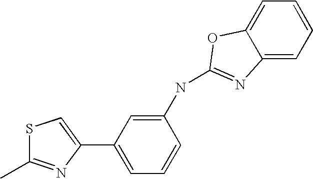 | | B |
| 93 | 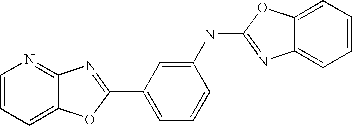 | D | |
| 94 | 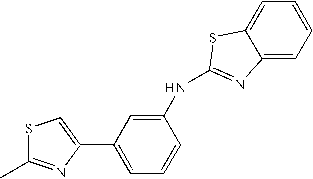 | D | |
| 95 | 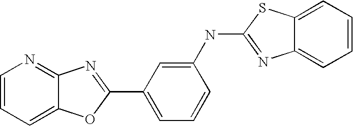 | NA | |
| 97 | 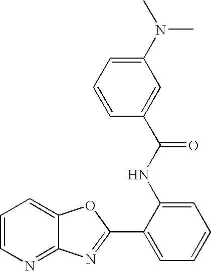 | A | |
| 98 | 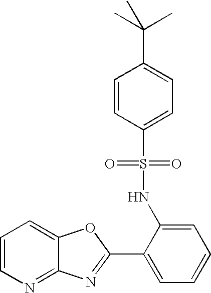 | NA | |
| 99 | 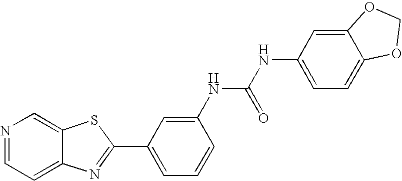 | A | |
| 100 | 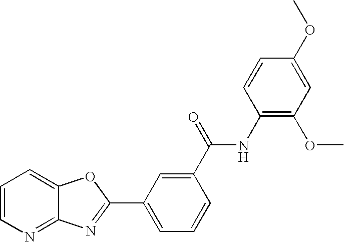 | A | |
| 101 | 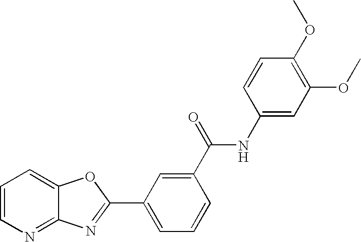 | C | |
| 102 | 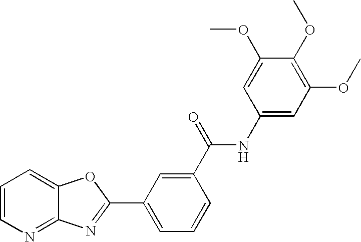 | A | |
| 103 | 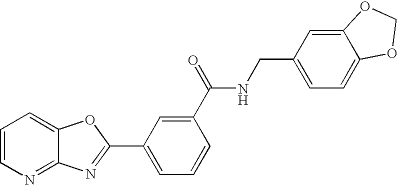 | NA | |
| 104 | 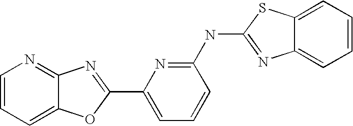 | A | |
| 105 | 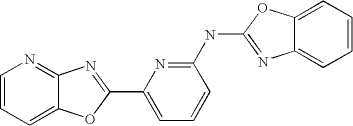 | A | |
| 133 | 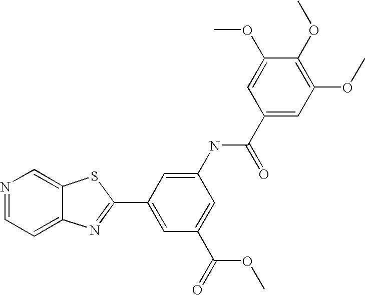 | NA | |
| 134 | 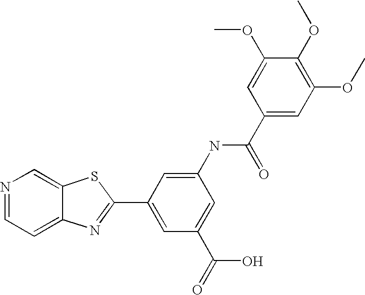 | NA | |

### 2. Modulators

In one embodiment, [symbol] -modulating compounds of the invention are represented by Structural Formula (I):

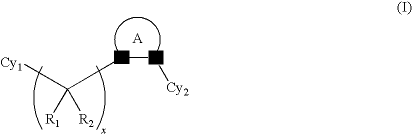

or a salt thereof, where:

Ring A is an optionally substituted, aliphatic, carbocyclic or heterocyclic ring;

R₁ and R₂ are independently halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂, or R₁ and R₂ taken together are ═O, ═S or ═N;

R₄ and R₅ are independently —H, a substituted or unsubstituted alkyl group, a substituted or unsubstituted aryl group or a substituted or unsubstituted non-aromatic heterocyclic group;

Cy₁ and Cy₂ are independently cyclic groups, one or both of which are optionally fused to Ring A, typically none or one is/are fused;

Y and Z are independently CH, N, O or S, provided that O and S are not adjacent to another O or S;

n is 1 or 2; and

x is 0 or 1.

In certain embodiments, Ring A is a heterocyclic ring, preferably further substituted and/or aliphatic (e.g., non-aromatic). In a particular embodiment, Ring A is a further substituted, aliphatic, heterocyclic ring. Ring A can be 5- to 8-membered, but is preferably 5- or 6-membered, more preferably 5-membered.

In certain embodiments, R₁ and R₂ taken together are ═O.

In certain embodiments, Y and/or Z are each CH. In particular embodiments, Y and Z are each CH, such as when R₁ and R₂ taken together are ═O.

In certain embodiments, Cy₁ and Cy₂ are each substituted or unsubstituted aryl groups, such as substituted or unsubstituted phenyl groups. In particular embodiments, Cy₁ and Cy₂ are each substituted or unsubstituted aryl groups when Y and Z are each CH and/or R₁ and R₂ taken together are ═O.

The stereochemistry of compounds represented by Structural Formula (I) is preferably as depicted in Structural Formula (II):

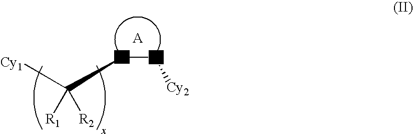

In another embodiment, [symbol] -modulating compounds of the invention are represented by Structural Formula (III):

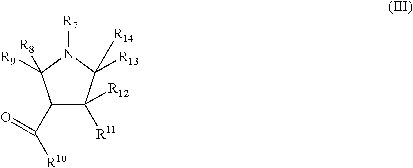

or a salt thereof, where:

R₄ and R₅ are independently —H, a substituted or unsubstituted alkyl group, a substituted or unsubstituted aryl group or a substituted or unsubstituted non-aromatic heterocyclic group;

R₇ is —H, a substituted or unsubstituted alkyl group, a substituted or unsubstituted aryl group, a substituted or unsubstituted non-aromatic heterocyclic group, a substituted or unsubstituted acyl group or a substituted or unsubstituted aminocarbonyl group, or R₇ and R₁₃ and R₁₄ taken together with the atoms to which they are attached are a substituted or unsubstituted heterocyclic ring;

R₈ is —H;

R₉ is a substituted or unsubstituted aryl group;

R₁₀ is a substituted or unsubstituted aryl group;

R₁₁ is —H;

R₁₂ is a substituted or unsubstituted aryl group;

R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and

n is 1 or 2.

In certain embodiments, R₁₃ is —CO₂R₄, such as —CO₂H.

In certain embodiments, R₁₂ is a substituted or unsubstituted alkyl group, such as an unsubstituted alkyl group. In particular embodiments, R₁₂ is a substituted or unsubstituted alkyl group and R₁₃ is —CO₂R₄.

In certain embodiments, R₁₀ and R₁₂ are independently substituted or unsubstituted phenyl or thienyl groups. Typically, R₁₀ is an unsubstituted phenyl group. Typically, R₁₂ is a nitrophenyl, methoxyphenyl, chlorophenyl or thienyl group.

In particular embodiments, R₁₀ and R₁₂ are independently substituted or unsubstituted phenyl groups and R₁₃ is —CO₂R₄.

In certain embodiments, R₉ is an aryl group other than a pyrazolyl group, for example, a substituted or unsubstituted phenyl or thienyl group, such as an alkoxyphenyl (e.g., methoxyphenyl) group. In particular embodiments, R₉ is a substituted or unsubstituted phenyl or thienyl group when R₁₀ and R₁₂ are independently substituted or unsubstituted phenyl groups and/or R₁₃ is —CO₂R₄.

In certain embodiments, R₉ is a substituted or unsubstituted pyrazolyl group.

In certain embodiments, —C(O)R₁₀ is located trans to —R₁₂.

In another embodiment, [symbol] -modulating compounds of the invention are represented by Structural Formula (IV):

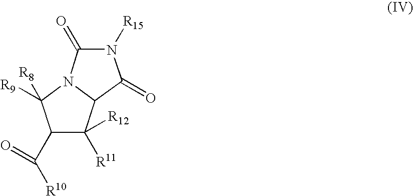

or a salt thereof, where:

R₄ and R₅ are independently —H, a substituted or unsubstituted alkyl group, a substituted or unsubstituted aryl group or a substituted or unsubstituted non-aromatic heterocyclic group;

R₈ is —H;

R₉ is a substituted or unsubstituted aryl group (e.g., 4-methoxy-3-nitrophenyl, nitrophenyl);

R₁₀ is a substituted or unsubstituted aryl or substituted or unsubstituted alkyl group (e.g., thienyl, phenyl, methyl);

R₁₁ is —H;

TABLE 3

| COMPOUND NO | STRUCTURE | ED₅₀ |
|---|---|---|
| 1 | 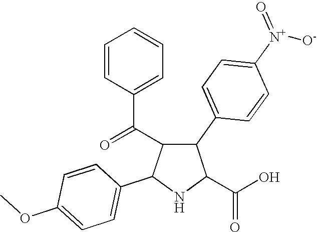 | A |
| 2 | 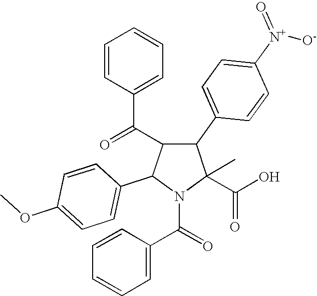 | B |
| 3 | 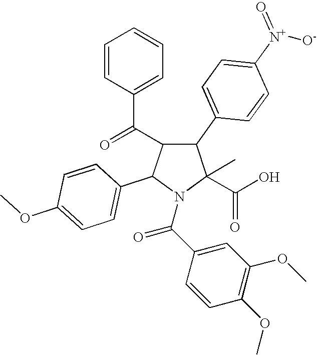 | A |
| 4 | 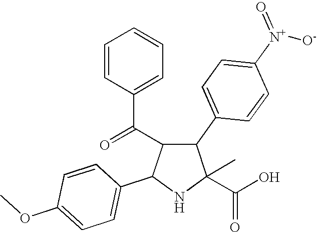 | A |
| 5 | 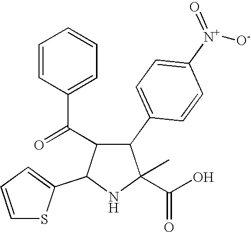 | B |
| 6 | 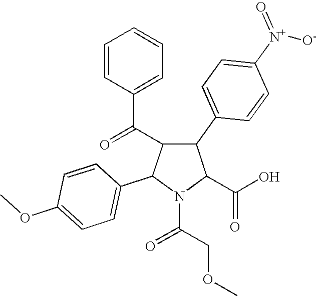 | B |

TABLE 5

| COMPOUND NO | STRUCTURE | ED₅₀ |
|---|---|---|
| 1 | 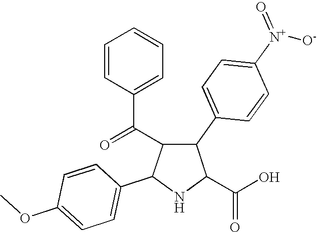 | A |
| 2 | 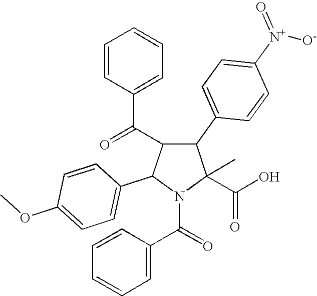 | D |
| 3 | 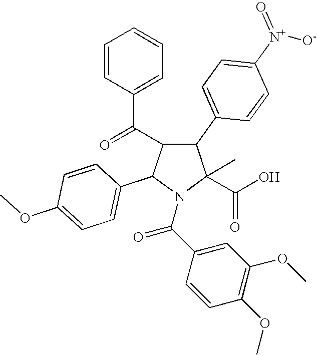 | D |
| 4 | 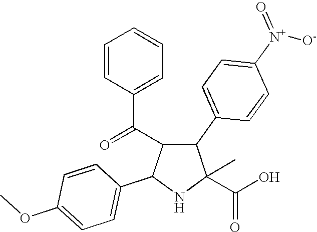 | A |
| 5 | 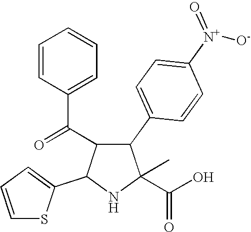 | D |
| 6 | 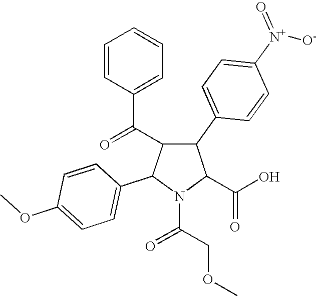 | A |
| 7 | 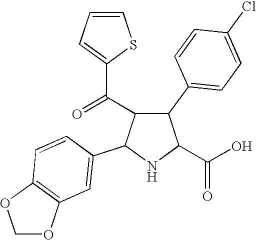 | D |
| 8 | 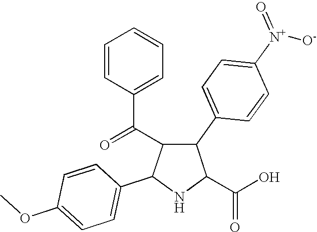 | B |
| 9 | 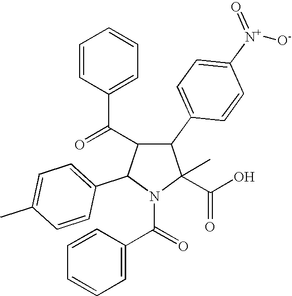 | C |
| 10 | 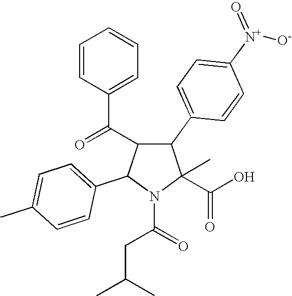 | C |
| 11 | 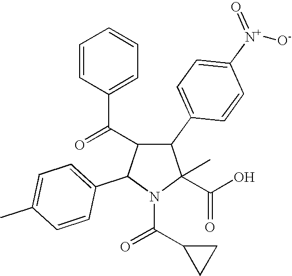 | C |
| 12 | 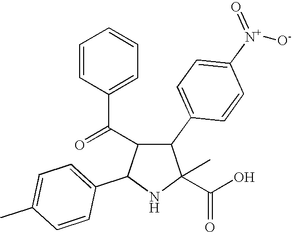 | B |
| 13 | 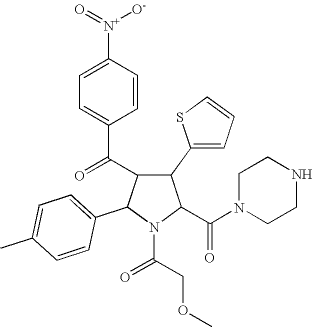 | D |
| 14 | 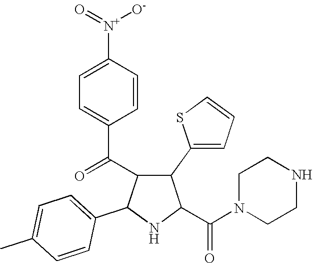 | B |
| 15 | 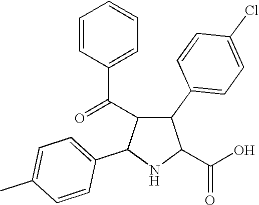 | C |
| 16 | 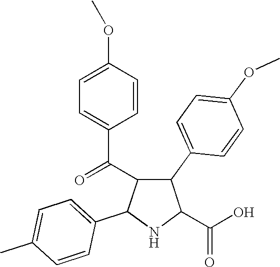 | C |
| 17 | 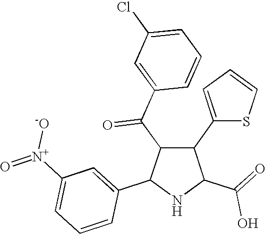 | D |

In one embodiment, [symbol] -modulating compounds of the invention are represented by Structural Formula (I):

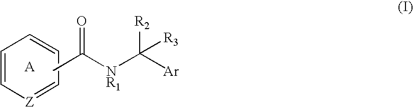

or a salt thereof, where:

Ring A is optionally substituted with one or more substituents in addition to the substituent indicated in Structural Formula (I):

Ar is a substituted or unsubstituted aryl group;

R₁ is —H, a substituted or unsubstituted alkyl group, a substituted or unsubstituted alkenyl group, a substituted or unsubstituted aryl group or a substituted or unsubstituted non-aromatic heterocyclic group;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₄ and R₅ are independently —H, a substituted or unsubstituted alkyl group, a substituted or unsubstituted aryl group or a substituted or unsubstituted non-aromatic heterocyclic group;

Z is CH or N; and

n is 1 or 2.

In certain embodiments, R₂ is a substituted alkyl group, such as a carboxyalkyl group (e.g., carboxymethyl).

In certain embodiments, Z is CH. In particular embodiments, Z is CH and R₂ is a substituted alkyl group.

In certain embodiments, R₁ and R₃ are each —H. In particular embodiments, R₁ and R₃ are each —H when Z is CH and/or R₂ is a substituted alkyl group.

In another embodiment, [symbol] -modulating compounds of the invention are represented by Structural Formula (II):

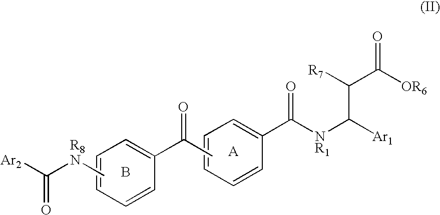

or a salt thereof, where:

Rings A and B are optionally substituted with one or more substituents in addition to the substituents indicated in Structural Formula (II):

Ar₁ and Ar₂ are independently a substituted or unsubstituted aryl group;

R₁, R₆ and R₈ are independently —H, a substituted or unsubstituted alkyl group, a substituted or unsubstituted aryl group or a substituted or unsubstituted non-aromatic heterocyclic group;

R₄ and R₅ are independently —H, a substituted or unsubstituted alkyl group, a substituted or unsubstituted aryl group or a substituted or unsubstituted non-aromatic heterocyclic group;

R₇ is selected from the group consisting of —H, a substituted or unsubstituted alkyl group, a substituted or unsubstituted aryl group, a substituted or unsubstituted non-aromatic heterocyclic group, halogen; —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and

n is 1 or 2.

In certain embodiments, Ar₁ is a substituted or unsubstituted phenyl or pyridyl group.

In certain embodiments, Ar₂ is a substituted or unsubstituted phenyl or thienyl group. In particular embodiments, Ar₁ is a substituted or unsubstituted phenyl or pyridyl group and Ar₂ is a substituted or unsubstituted phenyl or thienyl group.

In certain embodiments, one or more of R₁, R₆ and R₈ is —H. Preferably, R₁, R₆ and R₈ are each —H. In particular embodiments, R₁, R₆ and R₈ are each —H, Ar₁ is a substituted or unsubstituted phenyl or pyridyl group and/or Ar₂ is a substituted or unsubstituted phenyl or thienyl group. Preferred compounds of the invention are chosen such that R₁, R₆ and R₈ are each —H, Ar₁ is a substituted or unsubstituted phenyl or pyridyl group and Ar₂ is a substituted or unsubstituted phenyl or thienyl group.

Rings A and B typically have zero or one substituents in addition to those indicated in Structural Formula (II). Suitable substituents include halogens and lower alkyl groups.

One group of compounds of the invention encompassed by Structural Formula (II) are represented by Structural Formula (III)

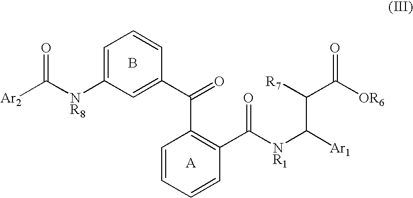

In yet another embodiment, [symbol] -modulating compounds of the invention are represented by Formula (IVa):

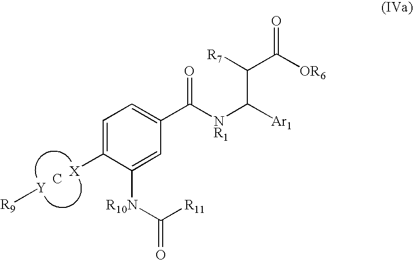

or a salt thereof, wherein:

Ring C is a substituted or unsubstituted 5-, 6- or 7-membered ring;

Ar₁ is a substituted or unsubstituted aryl group.

R₁, R₆ and R₁₀ are independently —H, a substituted or unsubstituted alkyl group, a substituted or unsubstituted aryl group or a substituted or unsubstituted non-aromatic heterocyclic group;

R₄ and R₅ are independently —H, a substituted or unsubstituted alkyl group, a substituted or unsubstituted aryl group or a substituted or unsubstituted non-aromatic heterocyclic group;

R₇ is selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₉ is —H, a substituted or unsubstituted acyl group, a substituted or unsubstituted aminocarbonyl group, a substituted or unsubstituted alkyl group, a substituted or unsubstituted aryl group or a substituted or unsubstituted non-aromatic heterocyclic group;

R₁₁ is —H, a substituted or unsubstituted alkyl group, a substituted or unsubstituted aryl group or a substituted or unsubstituted non-aromatic heterocyclic group;

X and Y are independently CH or N; and

n is 1 or 2.

Typically, at least one of X and Y is N, more typically, X and Y are each N.

Ring C is typically a 6- or 7-membered ring, particularly when X and Y are each N.

In certain embodiments, R₉ is a substituted or unsubstituted acyl group or —C(O)NR₁₂R₁₃. R₁₂ and R₁₃ are independently —H, a substituted or unsubstituted alkyl group, a substituted or unsubstituted aryl group or a substituted or unsubstituted non-aromatic heterocyclic group, or R₁₂ and R₁₃ taken together with the atom to which they are attached from a heterocyclic group. Typically, R₉ is an aryl-substituted acyl group or —C(O)NR₁₂R₁₃, wherein R₁₂ and R₁₃ taken together with the atom to which they are attached from a heterocyclic group.

In certain embodiments of the invention, R₁₁ is a substituted alkyl group, such as an aryloxyalkyl group (e.g., phenoxyalkyl, for example, phenoxymethyl), or a substituted aryl group. In particular embodiments, R₉ is a substituted or unsubstituted acyl group or —C(O)NR₁₂R₁₃ and R₁₁ is a substituted alkyl or aryl group.

In certain embodiments, Ar₁ is a substituted or unsubstituted phenyl, thienyl or pyridyl group. In particular embodiments, Ar₁ is a substituted or unsubstituted phenyl, thienyl or pyridyl group when R₉ is a substituted or unsubstituted acyl group or —C(O)NR₁₂R₁₃, and/or R₁₁ is a substituted alkyl or aryl group.

In certain embodiments, one or more of R₁, R₆, R₇ and R₁₀ is —H. Preferably, R₁, R₆, R₇ and R₁₀ are each —H. In particular embodiments, one or more of R₁, R₆, R₇ and R₁₀ is —H when Ar₁ is a substituted or unsubstituted phenyl, thienyl or pyridyl group, R₉ is a substituted or unsubstituted acyl group or —C(O)NR₁₂R₁₃, and/or R₁₁ is a substituted alkyl or aryl group.

A particular group of compounds encompassed by Structural Formula (IVa) are represented by Structural Formula (IV):

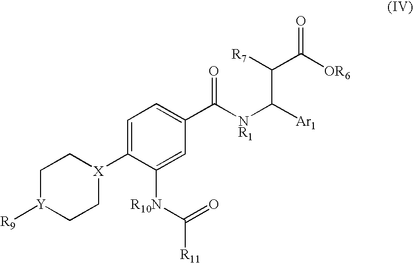

or a salt thereof, where:

Ar₁ is a substituted or unsubstituted aryl group;

R₁, R₆ and R₁₀ are independently —H, a substituted or unsubstituted alkyl group, a substituted or unsubstituted aryl group or a substituted or unsubstituted non-aromatic heterocyclic group;

R₄ and R₅ are independently —H, a substituted or unsubstituted alkyl group, a substituted or unsubstituted aryl group or a substituted or unsubstituted non-aromatic heterocyclic group;

R₇ is selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₉ is —H, a substituted or unsubstituted acyl group, a substituted or unsubstituted aminocarbonyl group, a substituted or unsubstituted alkyl group, a substituted or unsubstituted aryl group or a substituted or unsubstituted non-aromatic heterocyclic group;

R₁₁ is —H, a substituted or unsubstituted alkyl group, a substituted or unsubstituted aryl group or a substituted or unsubstituted non-aromatic heterocyclic group; X and Y are independently CH or N; and

n is 1 or 2.

In certain embodiments, R₉ is a substituted or unsubstituted acyl group or —C(O)NR₁₂R₁₃. R₁₂ and R₁₃ are independently —H, a substituted or unsubstituted alkyl group a substituted or unsubstituted aryl group or a substituted or unsubstituted non-aromatic heterocyclic group, or R₁₂ and R₁₃ taken together with the atom to which they are attached from a heterocyclic group. Typically, R₉ is an aryl-substituted acyl group or —C(O)NR₁₂R₁₃, wherein R₁₂ and R₁₃ taken together with the atom to which they are attached from a heterocyclic group. Particular examples of R₉ are

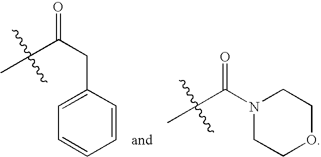

In certain embodiments of the invention, R₁₁ is a substituted alkyl group, such as an aryloxyalkyl group (e.g., phenoxyalkyl, for example, phenoxymethyl). In particular embodiments, R₉ is a substituted or unsubstituted acyl group or —C(O)NR₁₂R₁₃ and R₁₁ is a substituted alkyl group.

In certain embodiments, Ar₁ is a substituted or unsubstituted phenyl or pyridyl group. In particular embodiments, Ar₁ is a substituted or unsubstituted phenyl or pyridyl group when R₉ is a substituted or unsubstituted acyl group or —C(O)NR₁₂R₁₃, and/or R₁₁ is a substituted alkyl group.

In certain embodiments, one or more of R₁, R₆, R₇ and R₁₀ is —H. Preferably, R₁, R₆, R₇ and R₁₀ are each —H. In particular embodiments, one or more of R₁, R₆, R₇ and R₁₀ is —H when Ar₁ is a substituted or unsubstituted phenyl or pyridyl group, R₉ is a substituted or unsubstituted acyl group or —C(O)NR₁₂R₁₃, and/or R₁₁ is a substituted alkyl group.

In certain embodiments, at least one of X and Y is N. Preferably, both X and Y are N. In particular embodiments, at least one of X and Y is N when one or more of R₁, R₆, R₇ and R₁₀ is —H, Ar₁ is a substituted or unsubstituted phenyl or pyridyl group, R₉ is a substituted or unsubstituted acyl group or —C(O)NR₁₂R₁₃, and/or R₁₁ is a substituted alkyl group. Preferred compounds are selected such that at least one of X and Y is N, one or more of R₁, R₆, R₇ and R₁₀ is —H, Ar₁ is a substituted or unsubstituted phenyl or pyridyl group, R₉ is a substituted or unsubstituted acyl group or —C(O)NR₁₂R₁₃, and R₁₁ is a substituted alkyl group.

In certain embodiments, the compounds of the invention exclude those species disclosed in the Tables 3 and 4.

Novel compounds of the invention can also be used in the methods described above.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.
[symbol] modulating compounds of the invention having hydroxyl substituents, unless otherwise indicated, also include the related secondary metabolites, particularly sulfate, phosphate, acyl (e.g., acetyl, fatty acid acyl) and sugar (e.g., glucurondate, glucose) derivatives. In other words, substituent groups —OH also include —OSO3− M+, where M+ is a suitable cation (preferably H+, NH4+ or an alkali metal ion such as Na+ or K+) and sugars such as

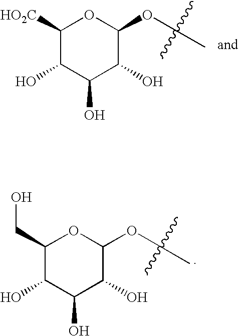

These groups are generally cleavable to —OH by hydrolysis or by metabolic (e.g., enzymatic) cleavage.
[symbol] modulating compounds of the invention advantageously [symbol] modulate the level and/or activity of a protein.

Separately or in addition to the above properties, certain [symbol] modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8.

Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A 5- to 7-membered ring includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOR^{a}, —COOR^{a}, —OCO₂R^{a}, C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(—NR^{c})—N(R^{a}R^{b}), —NR^{d}—C(═NH)—NH₂, —C(═NR^{c})—NH₂, —NH—C(═NR^{c})—NHR^{a}, —NH—C(═NR^{c})—N(R^{a}R^{b}), —NR^{d}H—C(═NH)—NH₂, —NHR^{a}, —NR^{d}—C(═NR^{c})—N((R^{a}R^{b}), —NHNH₂, NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}—R^{d} are each independently an aliphatic, substituted aliphatic, benzyl, substituted benzyl.

TABLE 3

| COMPOUND NO | STRUCTURE | ED₅₀ |
|---|---|---|
| 1 | 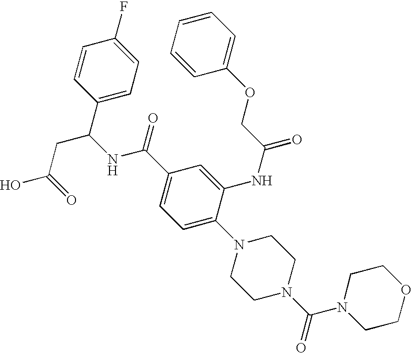 | A |
| 2 | 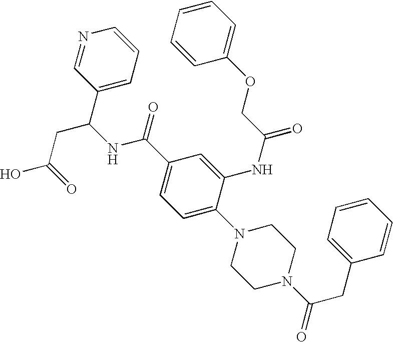 | B |
| 3 | 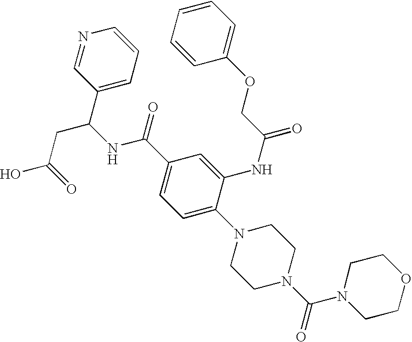 | B |
| 4 | 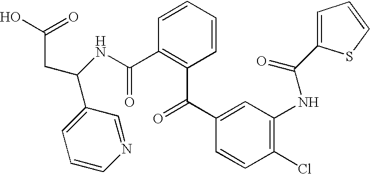 | A |

TABLE 4

| COMPOUND NO | STRUCTURE | ED₅₀ |
|---|---|---|
| 2 | 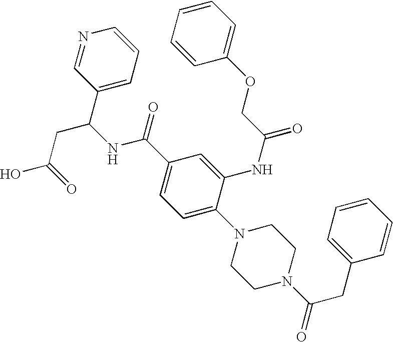 | B |
| 4 | 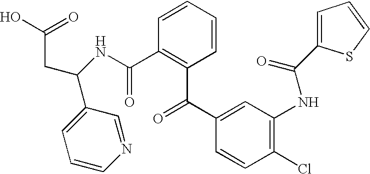 | B |
| 5 | 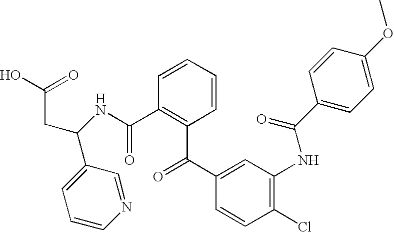 | A |
| 6 | 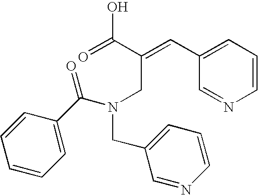 | C |
| 7 | 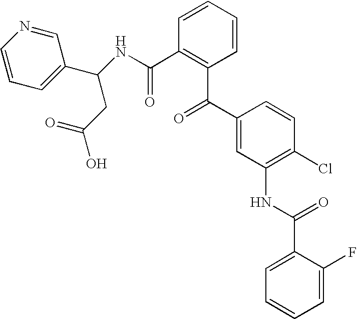 | A |
| 8 | 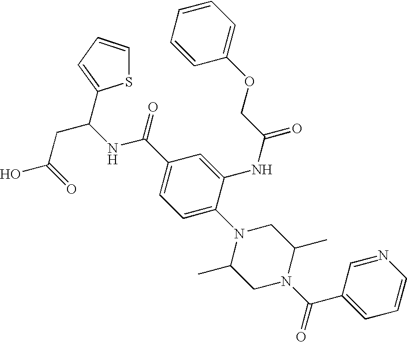 | C |
| 9 | 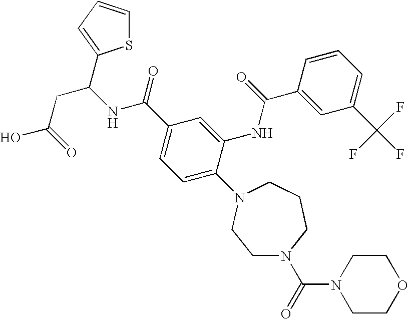 | B |
| 10 | 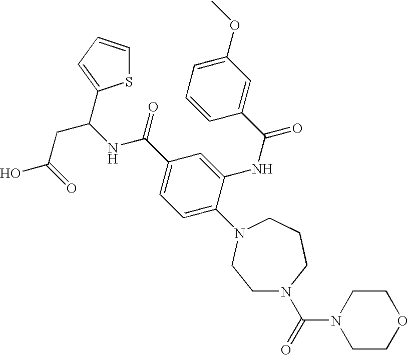 | D |
| 11 | 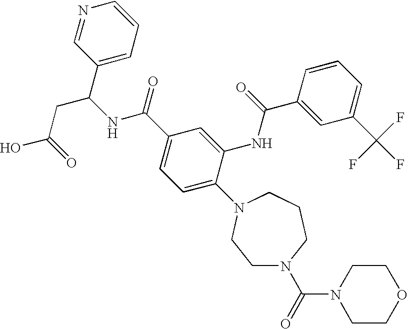 | B |

### 2. Modulators

In one embodiment, [symbol] compounds of the invention are represented by Structural Formula (I):

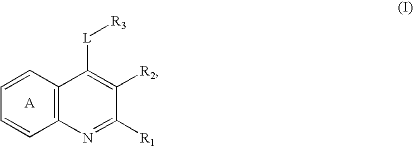

or a salt thereof, where, as valence permits:

Ring A is optionally substituted;

R₁ and R₂ are independently selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂, or R₁ and R₂ taken together with the atoms to which they are attached form an optionally substituted ring,

L is selected from —CH═CH—C(O)—, —CH₂—N(R₄)—C(O)—, —C(O)—CH₂—, —C(O)NR₄—, —C(O)—N(R₄)—C(O)—, —C(O)—N(R₄)—N(R₅)—, —C(O)—N(R₄)—N(R₅)—C(O)—, —CH₂—N(R₄)—N(R₅)—, —N(R₄)—S(O)₂, —S(O)₂—N(R₄)—, —N(R₄)—N(R₅)—C(O)—, —N(R₄)—N(R₅)—CH₂, —N(R₄)—N(R₅)— or

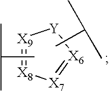

R₃, R₄ and R₅ are, independently for each occurrence, —H, a substituted or unsubstituted alkyl group, a substituted or unsubstituted aryl group or a substituted or unsubstituted non-aromatic heterocyclic group;

Y is selected from O, S, or NR₄;

each of X₆, X₇, X₈ and X₉ is independently selected from CR₇, C, or N, wherein at least two of X₆, X₇, X₈ or X₉ are not N;

each R₇ is independently selected from H or (C₁-C₃)-straight or branched alkyl; and

n is 1 or 2.

In certain embodiments, L is —NR₄—, —C(O)O—, —C(O)NR₄—, —NR₄C(O)—, —NR₄—NR₅—C(O)—, —C(O)—NR₄—NR₅— or —CHR₄═CHR₅—. In certain such embodiments, L is —C(O)NR₄—, —NR₄C(O)—, —NR₂—NR₅—C(O)—, —C(O)—NR₄—NR₅— or —CHR₄═CHR₅—.

In certain embodiments, R₄ or R₅ when it appears in L is selected from H and (C₁-C₃)-straight or branched alkyl. In certain embodiments, R₄ and R₅ when they appear in L are H.

In certain embodiments, R₂ is selected from —H and —OH. In certain embodiments, R₂ is —H.

In certain embodiments, R₃ is a substituted or unsubstituted non-aromatic heterocyclic group or a substituted or unsubstituted aryl group, such as a substituted or unsubstituted heteroaryl group.

In certain embodiments, R₃ is an alkyl group substituted with a substituted or unsubstituted non-aromatic heterocyclic group or an alkyl group substituted with a substituted or unsubstituted aryl group.

In certain embodiments, R₁ and R₂ taken together with the atoms to which they are attached form an optionally substituted ring. In particular embodiments, the optionally substituted ring is aromatic, such as a 6-membered aromatic ring.

In certain embodiments, R₁ is a substituted or unsubstituted aryl group, such as a substituted or unsubstituted heteroaryl group.

In certain embodiments, R₁ is a substituted or unsubstituted alkyl group, such as a methyl or ethyl group.

In certain embodiments, Ring A is unsubstituted. An exemplary embodiment is where Ring A is unsubstituted and R₁ is a substituted or unsubstituted aryl group.

In certain embodiments, Ring A is substituted, such as with a substituted or unsubstituted alkyl group. An exemplary embodiment is where Ring A is substituted and R₁ is a substituted or unsubstituted alkyl group.

In another embodiment, [symbol] -modulating compounds of the invention are represented by Structural Formula (V):

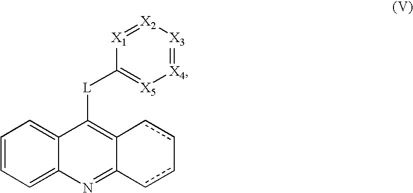

wherein, as valence permits:

each of X₁, X₂, X₃, X₄ and X₅ is independently selected from N or CR₆, wherein no more than two of X₁, X₂, X₃, X₄ or X₅ are N;

each R₆ is independently selected from H, —OCH₃, —CH₃, or —CF₃;

L is selected from —CH═CH—C(O)—, —CH₂—N(R₄)—C(O)—, —C(O)—CH₂—, —C(O)—N(R₄)—, —C(O)—N(R₄)—CH₂—, —C(O)—N(R₄)—CH₂—CH, —C(O)—N(R₄)—C(O)—, —C(O)—N(R₄)—N(R₅)—, —CH₂—N—N(R—N(R₅)—, —N(R₄)—S(O)₂—, —S(O)₂—N(R₄)—, —N(R₄)—N(R₅)—C(O)—, —C(O)N(R₄)—N(R₅)—C(O)—, —N(R₄)—N(R₅)—CH₂, —N(R₄)—N(R₅)—,

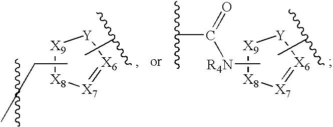

each of R₄ and R₅ is independently selected from H or CH₃;

Y is selected from O, S, or NR₄;

each of X₆, X₇, X₈ and X₉ is independently selected from CR₇, C, or N, wherein at least two of X₆, X₇, X₈ or X₉ are not N;

each R₇ is independently selected from H or (C₁-C₃)-straight or branched alkyl; and

the hashed bonds are either simultaneously present or simultaneously absent

In certain embodiments, when the hashed bonds are simultaneously present, L is —N(R₄)—N(R₅)—C(O)—, and each of X₂, X₃, and X₄ are —OCH₃, then R₄ is hydrogen.

In certain embodiments, when the hashed bonds are simultaneously absent and L is —N(R₄—N(R₅)—C(O)—, both X₁ and X₅ are CR₆—.

In certain embodiments, L is selected from —C(O)—N(R₄)—N(R₅)—, —CH₂—N(R₄)—N(R₅)—, —N(R₄)—N(R₅)—C(O—, —N(R₄)—N(R₅)—,

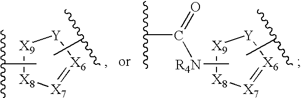

particularly —NH—NH—C(O)—, —NH—NH —, —N(CH₃)—NH—C(O)—, —CH₂—NH—NH—, —C(O)—NH—NH—,

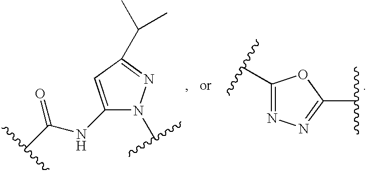

In certain embodiments, such as when L has one of the values described above, no more than one of X₁, X₂, X₃, X₄ and X₅ is N, for example, exactly one of X₁, X₂, X₃, X₄ and X₅ is N. In certain such embodiments, each of X₁, X₂, X₃, X₄ and X₅ is selected from N or CH. In other such embodiments, each of X₁, X₂, X₃, X₄ and X₅ is CR₆, such as where each R₆ is hydrogen. In a particular embodiment, X₁ and X₅ are CH and each of X₂, X₃, and X₄ is C—OCH₃.

In another embodiment, [symbol] -modulating compounds of the invention are represented by Structural Formula (II):

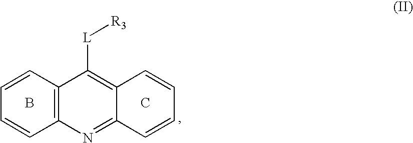

or a salt thereof, wherein:

Rings B and C are independently optionally substituted;

L is —NR₄R₅, —C(O)O—, —C(O)NR₄—, —NR₄C(O)—, —NR₄—NR₅—C(O)—, —C(O)—NR₄—NR₅— or —CHR₄═CHR₅—; and

R₃, R₄ and R₅ are independently —H, a substituted or unsubstituted alkyl group, a substituted or unsubstituted aryl group or a substituted or unsubstituted non-aromatic heterocyclic group.

In certain embodiments, L is —C(O)—NR₄—NR₅—.

In certain embodiments, R₃ is a substituted or unsubstituted aryl group. In particular embodiments, L is —C(O)—NR₄—NR₅— and R₃ is a substituted or unsubstituted aryl group. Particular R₃ groups are substituted or unsubstituted phenyl or pyridyl groups, such as a pyridyl or an alkoxy-substituted phenyl group (e.g., a trialkoxy-substituted phenyl group such as 3,4,5-trimethoxyphenyl).

In certain embodiments, Ring B and/or Ring C is unsubstituted. Preferably, both Rings B and C are unsubstituted, such as when L is —C(O)—NR₄—NR₅— and/or R₃ is a substituted or unsubstituted aryl group.

In certain embodiments, R₄ and/or R₅ are —H. Preferably, both R₄ and R₅ are —H. In particular embodiments, Rings B and C are unsubstituted when L is —C(O)—NR₄—NR₅— and/or R₃ is a substituted or unsubstituted aryl group.

In certain embodiments, R₂ is selected from —H and —OH. In certain embodiments, R₂ is —H.

In yet another embodiment, [symbol] -modulating compounds of the invention are represented by Structural Formula (III):

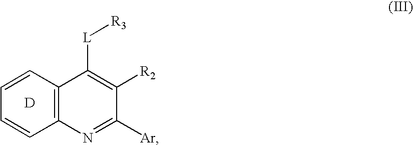

or a salt thereof, wherein, as valence permits:

Ring D is optionally substituted;

Ar is a substituted or unsubstituted aryl group;

R₂ is selected from —H, a substituted or unsubstituted alkyl group, a substituted or unsubstituted aryl group, a substituted or unsubstituted non-aromatic heterocyclic group, halogen, —OR₄—CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

L is selected from —C(O)O—, —C(O)—, —C(O)N(R₄)—, —C(O)—N(R₄)—C(O)—, —C(O)—N(R₄)—N(R₅)—, —C(O)—N(R₄)—N(R₅)—C(O)—, —C(O)—N(R₄)—S(O)₂—, —N(R₄)C(O)—, —N(R₄)—S(O)₂—, —N(R₄)—S(O)₂—N(R₅)—, —N(R₄)*R₅)—, —N(R₄)—N(R₅)—C(O)—, —N(R₄)—C(O)—N(R₅)—, —N(R₄)—C(O)—N(R₅)—S(O)₂, —N(R₄)—C(S)—N(R₅)—, —N(R₄)—C(O)—CH₂—N(R₅)—, —N(R₄)—C(O)—CH═C(CH₃)—, —N(R₄)—C(═N—CN)—N(R₅)—, —N(R₄)—C(═NH)—N(R₅)—, —N(R₄)—, —N(R₄)—CH₂—C(O)—N(R₅)—, —CH₂—, —CH₂—N(R₄)—C(O)—, —CH₂—C(O)—N(R₄)—, —CH(R₄)═CH(R₅)—, —CH═CH—C(O)—, N—N N/70 —N(R₄)—N(R₅)—, CH₂—N(R₄)—N(R₅)—, —S(O)—N(R₄)—,

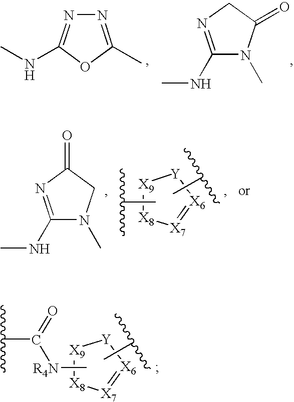

such as —NR₄R₅, —C(O)—, —C(O)NR₄—, —NR₄C(O)—, —NR₄—NR₅—C(O)—, —C(O)—NR₄—NR₅— or —CHR₄═CHR₅—;

each of R₃, R₄ and R₅ is independently selected from —H, a substituted or unsubstituted alkyl group, a substituted or unsubstituted aryl group or a substituted or unsubstituted non-aromatic heterocyclic group;

Y is selected from O, S, or NR₄;

each of X₆, X₇, X₈ and X₉ is independently selected from CR₇, C, or N, wherein at least two of X₆, X₇, X₈ or X₉ are not N;

each R₇ is independently selected from H or (C₁-C₃)-straight or branched alkyl, and

n is 1 or 2.

In certain embodiments, R₄ or R₅ when it appears in L is selected from H and (C₁-C₃)-straight or branched alkyl. In certain embodiments, R₄ and R₅ when they appear in L are H.

In certain embodiments, R₂ is selected from H and OH. In certain embodiments, R₂ is H.

In certain embodiments, R₃ is a substituted or unsubstituted non-aromatic heterocyclic group or a substituted or unsubstituted aryl group, such as a substituted or unsubstituted heteroaryl group.

In certain embodiments, R₃ is selected from —H, Cyc or (C₁-C₂) alkylene-Cyc, wherein when R₃ is —H, L is —C(O)O—; Cyc is selected from a substituted aryl group, an unsubstituted aryl group, a substituted non-aromatic heterocyclic group or an unsubstituted non-aromatic heterocyclic group; and each of R₄ and R₅ is independently selected from —H or —CH₃. In certain such embodiments, L and R₃ taken together form a moiety selected from C(O)—OH, C(O)—N(R₄)-Cyc, C(O)—N(R₄)—(CH₂)-Cyc, N(R₄)—N(R₅)—C(O)-Cyc, N(R₄)—N(R₅)-Cyc, CH₂—N(R₄)—N(R₅)-Cyc, C(O)—N(R₄)—N(R₅)-Cyc, or

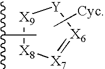

In particular embodiments, L and R₃ taken together form a moiety selected from —C(O)—OH, —C(O)—NH—(CH₂)ₙ-Cyc, —C(O)—NH-Cyc, —NH—NH—C(O)-Cyc, —NH—NH-Cyc, —N(CH₃)—NH—C(O)-Cyc, —CH₂—NH—NH—-Cyc, —C(O)—NH—NH-Cyc, or

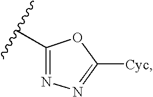

preferably —C(O)—OH, —C(O)—NH—(CH₂)ₙ-Cyc, —C(O)—NH-Cyc, or NH—NH—C(O)-Cyc, such as —C(O)—NH—(CH₂)ₙ-Cyc where Cyc is unsubstituted. Typically, Cyc is selected from pyridyl or morpholino. In other particular embodiments, L and R₅ taken together form —NH—NH—C(O)-Cyc and Cyc is phenyl.

In particular embodiments, when L and R₃ are taken together to form C(O)—N(R₄)-Cyc, and Cyc is phenyl, said phenyl is monosubstituted with morpholino.

In particular embodiments, when L and R₃ are taken together to form N(R₄)—N(R₅)—C(O)-Cyc and Cyc is a substituted phenyl, said substituted phenyl is not 3,4,5 trimethoxyphenyl or 4-N,N dimethylaminophenyl.

In particular embodiments, when L and R₃ are taken together to form C(O)—N(R₄)—(CH₂)₂-Cyc, Cyc is not piperidinyl or piperazinyl.

In particular embodiments, when L and R₃ are taken together to form C(O)—N(R₄)—(CH₂)₂-Cyc and Cyc is morpholino, Ar is not furanyl.

In certain embodiments, Ar is unsubstituted. In certain such embodiments, Ar is selected from phenyl, pyridyl, thienyl, or furanyl.

In certain embodiments, ring D is unsubstituted or monosubstituted, particularly when Ar is selected from phenyl, pyridyl, thienyl, or furanyl. When ring D is monosubstituted, the substituent is typically at the 6-position of the ring system. Typical substituents for ring D include a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂. Preferred substituents include methyl and halo.

In certain embodiments, L is —C(O)NR₄—. In certain such embodiments, R₃ is a substituted or unsubstituted heteroaryl group having at least one ring nitrogen atom or a substituted or unsubstituted non-aromatic heterocyclic group having at least one nitrogen atom and or a C₁₋₂ alkylene (e.g., unsubstituted alkylene) group substituted by substituted or unsubstituted heteroaryl group having at least one ring nitrogen atom or a substituted or unsubstituted non-aromatic heterocyclic group having at least one nitrogen atom.

In certain embodiments, L is —NR₄R₅—.

In certain embodiments, R₃ is a substituted alkyl group or a cyclic group. When R₃ is a substituted alkyl group, it is preferably substituted with a cyclic group. When R₃ is a cyclic group, it is preferably an aryl group (e.g., phenyl) or a non-aromatic heterocyclic group (e.g., morpholino). In a particular embodiment, L is —C(O)NR₄— and R₃ is a substituted alkyl group or a cyclic group. When R₃ is a cyclic group or an alkyl group substituted with a cyclic group, the cyclic group is typically a phenyl or pyridyl group that is unsubstituted or substituted only at one or both of the positions adjacent to where R₃ attaches to L.

In other certain embodiments, R₃ is a cyclic group substituted at least one position that is not adjacent to the atom by which R₃ attaches to L. For example, if R₃ is a phenyl or pyridyl group, at least one substituent is meta or para to the atom where R₃ attaches to L.

In certain embodiments, R₁ is a substituted or unsubstituted heteroaryl group, such as a thienyl or furanyl group. In particular embodiments, R₁ is a substituted or unsubstituted heteroaryl group, such as a thienyl or furanyl group, when L is —C(O)NR₄— and/or R₃ is a substituted alkyl group or a cyclic group. For example, R₁, R₃ and L can have these values when R₃ is a cyclic group or an alkyl group substituted with a cyclic group, the cyclic group is typically a phenyl or pyridyl group that is unsubstituted or substituted only at one or both of the positions adjacent to where R₃ attaches to L.

In certain embodiments, Ring D is unsubstituted or is substituted with a halogen (e.g., Cl, Br, F, I) or an unsubstituted alkyl group (e.g., methyl, ethyl, propyl). In particular embodiments, Ring D is unsubstituted or is substituted with a halogen or an unsubstituted alkyl group when R₁ is a substituted or unsubstituted heteroaryl group, L is —C(O)NR₄— and/or R₃ is a substituted alkyl group or a cyclic group. In other particular embodiments, Ring D is substituted with a halogen or an unsubstituted alkyl group when R₁ is a substituted or unsubstituted heteroaryl group or a substituted or unsubstituted alkyl group, L is —C(O)NR₄— or —NR₄R₅— and/or R₃ is a substituted alkyl group or a cyclic group.

One group of compounds encompassed by Structural Formula (III) are represented by the formula:

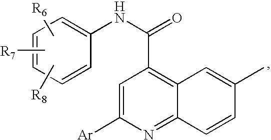

wherein:

Ar is selected from phenyl

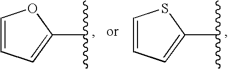

such as

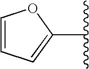

or

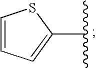

and

each of R₆, R₇, and R₅ is independently selected from —H, —CF₃, —C1-C3 straight or branched alkyl, —O—(C1-C3 straight or branched alkyl), —O—CF₃, —N(C1-C3 straight or branched alkyl)₂, halo, morpholino, —(C1-C3 straight or branched alkyl)-morpholino, piperazinyl, —(C1-C3 straight or branched alkyl)-piperazinyl, -piperazinyl, —NH—S(O)₂—(C1-C3 straight or branched alkyl), or —NH—S(O)₂-phenyl, wherein said phenyl, piperazinyl or morpholino is optionally substituted with methyl. In —N(C1-C3 straight or branched alkyl)₂, the two alkyl groups may be the same or different.

In certain embodiments, the compound is not

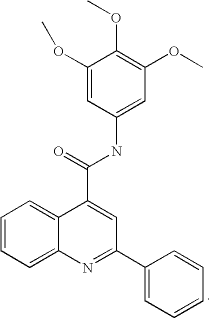

In certain embodiments, at least one of R₆, R₇, or R₈ is not —H. In certain such embodiments, zero or one of R₆, R₇, or R₈ is morpholino, —(C1-C3 straight or branched alkyl)-morpholino, piperazinyl, —(C1-C3 straight or branched alkyl)-piperazinyl, -piperazinyl, or —NH—S(O)₂-phenyl, wherein said phenyl, piperazinyl or morpholino is optionally substituted with methyl. In particular embodiments, R₆ is morpholino, —(C1-C3 straight or branched alkyl)-morpholino, piperazinyl, —(C1-C3 straight or branched alkyl)-piperazinyl, -piperazinyl, or —NH—S(O)₂-phenyl, wherein said phenyl, piperazinyl or morpholino is optionally substituted with methyl, and R₇ and R₈ are hydrogen. In other particular embodiments, each of R₆, R₇, and R₈ is independently selected from —H, —CF₃, -methyl, —O-methyl, —O—CF₃, —N(CH₃)₂, fluoro, morpholino, —CH₂—CH₂-morpholino, piperazinyl-CH₃, —NH—S(O)₂—CH₃, or —NH—S(O)₂-phenyl-CH₃.

In certain embodiments, L is selected from —C(O)O—, —C(O)—, —C(O)—N(R₄)—C(O)—, —C(O)—N(R₄)—N(R₅)—, —C(O)—N(R₄)—S(O)₂—, —N(R₄)C(O)—, —N(R₄)—S(O)₂—, —N(R₄)—S(O)₂—N(R₅), —N(R₄)(R₅)—, —N(R₄)—N(R₅)—C(O)—, —N(R₄)—C(O)—N(R₅)—, —N(R₄)—C(O)—N(R₅)—S(O)₂, —N(R₄)—C(S)—N(R₅)—, —N(R₄)—C(O)—CH₂—N(R₅)—, N(R₄)—C(O)—CH═C(CH₃)—, —N(R₄)—C(═N—CN)—N(R₅)—, —N(R₄)—C(═—NH)—N(R₅)—, —N(R₄)—, N(R₄)—CH₂—C(O)—N(R₅)—, —CH₂—, —CH₂—N(R₄)—C(O)—, CH₂—C(O)—N(R₄)—, —CH(R₄)═CH(R₅)—, —CH═CH—C(O)—, —N(R₄)—N(R₅)—, —CH₂—N(R₄)—N(R₅)—, —S(O)₂—N(R₄)—,

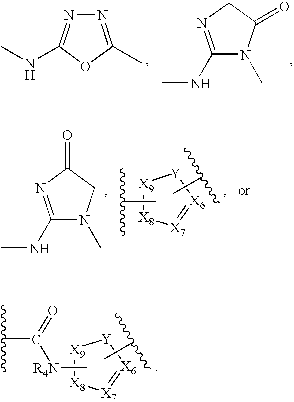

A particular group of compounds of the invention encompassed by Structural Formula (III) are represented by Structural Formula (IV):

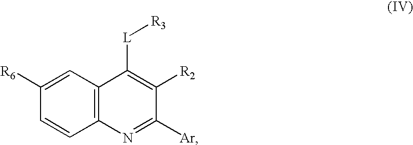

or a salt thereof, where R₆ is selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, SR₄, —OSO₃H, —S(O)R₄, —S(O)ₙOR₄, —S(O)ₙNR₄R₅, —NR₄R₅, —NR₆C(O)OR₅, —NR₄C(O)R₅ and —NO₂. Preferred values of R₆ are a halogen or an unsubstituted alkyl group.

Suitable values of Ar, L, R₂ and R₃ are as described above.

In certain embodiments, the compounds of the invention exclude Compounds 1-11, as shown in the Examples below.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.
[symbol] modulating compounds of the invention having hydroxyl substituents, unless otherwise indicated, also include the related secondary metabolites, such as phosphate, sulfate, acyl (e.g., acetyl, fatty acid acyl) and sugar (e.g., glucuronate, glucose) derivatives. In other words, substituent groups —OH also include —OSO₃⁻M⁺, where M⁺ is a suitable cation (preferably H⁺, NH₄⁺ or an alkali metal ion such as Na⁺ or K⁺) and sugars such as

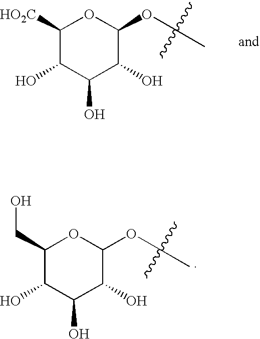

These groups are generally cleavable to —OH by hydrolysis or by metabolic (e.g., enzymatic) cleavage.

[symbol] -modulating compounds of the invention advantageously modulate the level and/or activity of a protein.

Separately or in addition to the above properties, certain sirtuin-modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity.

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8.

Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}—C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}—CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c}), —N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}C(═NR^{c})—NH₂, —NR^{d}.

TABLE 4

| COMPOUND NO | STRUCTURE | ED₅₀ |
|---|---|---|
| 1 | 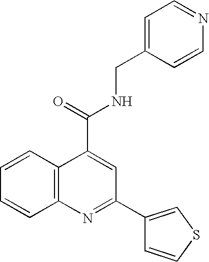 | A |
| 2 | 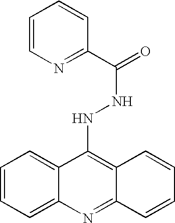 | B |
| 3 | 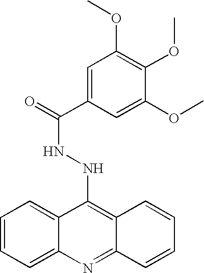 | A |
| 4 | 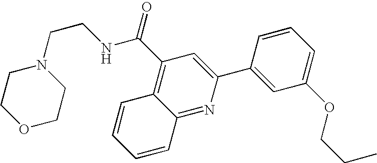 | D |
| 5 | 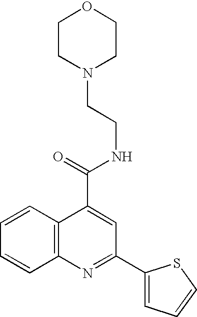 | C |
| 6 | 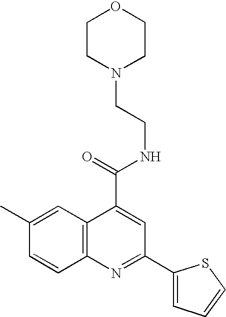 | A |
| 7 | 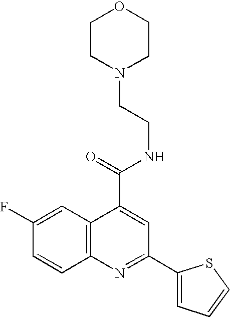 | C |
| 8 | 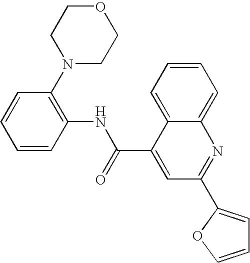 | A |
| 9 | 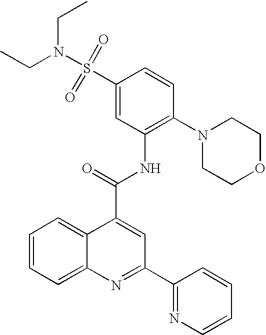 | D |
| 10 | 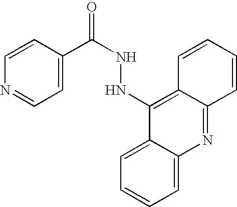 | A |
| 11 | 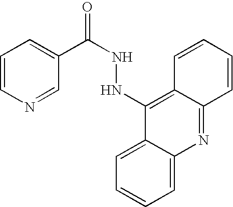 | A |
| 15 | 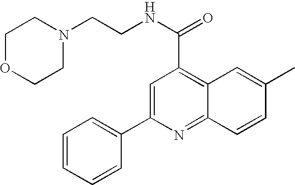 | B |
| 16 | 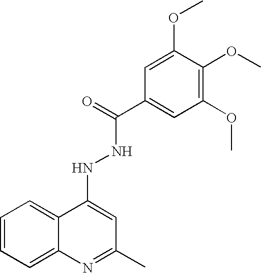 | D |
| 17 | 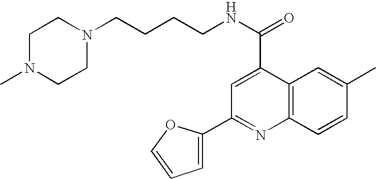 | D |
| 18 | 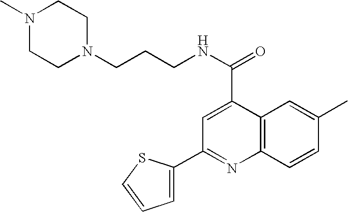 | D |
| 19 | 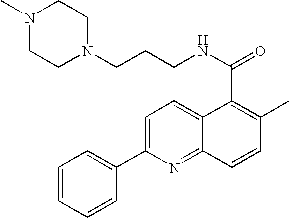 | D |
| 20 | 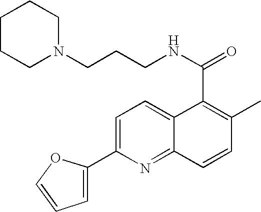 | D |
| 21 | 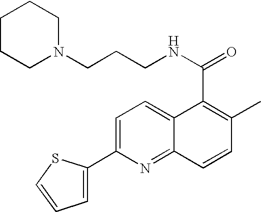 | D |
| 22 | 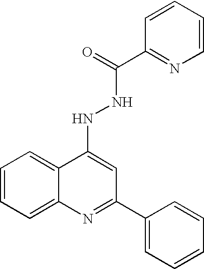 | A |
| 23 | 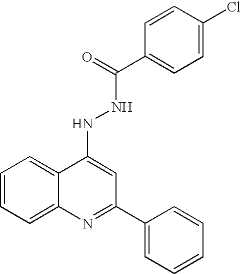 | A |
| 24 | 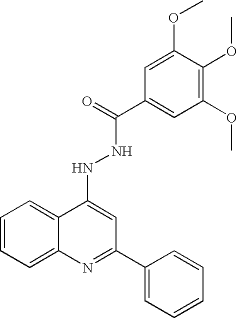 | D |
| 25 | 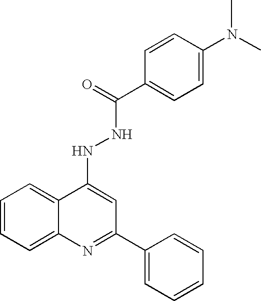 | D |
| 26 | 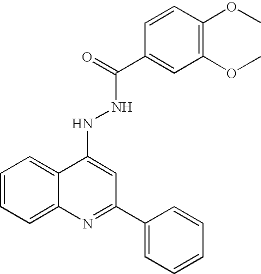 | A |
| 27 | 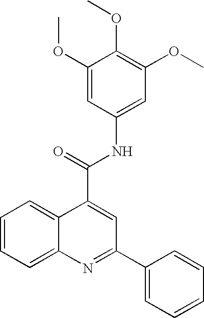 | D |
| 28 | 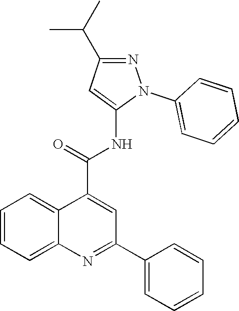 | D |
| 29 | 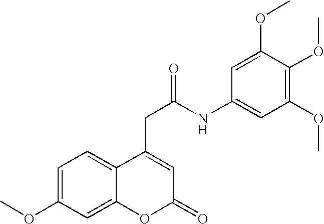 | D |
| 30 | 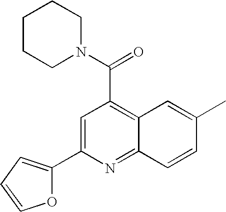 | D |
| 31 | 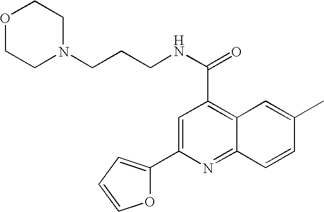 | D |
| 32 | 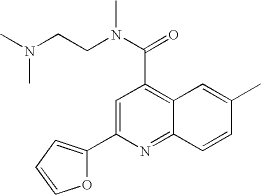 | D |
| 33 | 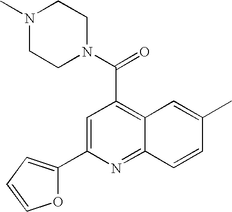 | D |
| 34 | 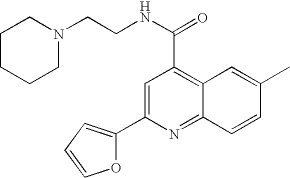 | D |
| 35 | 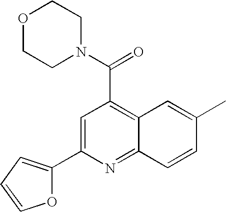 | D |
| 36 | 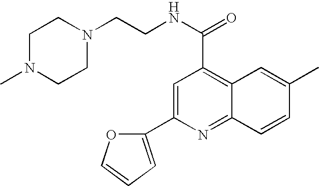 | |
| 37 | 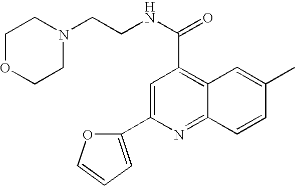 | |
| 38 | 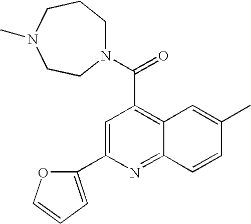 | |
| 39 | 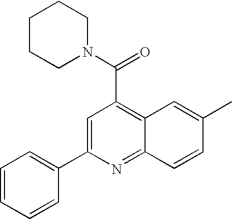 | |
| 40 | 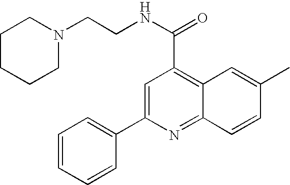 | |
| 41 | 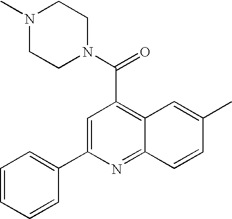 | |
| 42 | 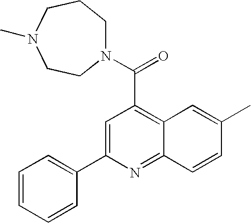 | |
| 43 | 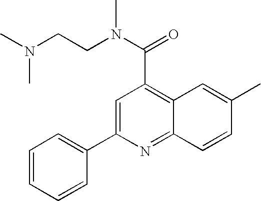 | |
| 44 | 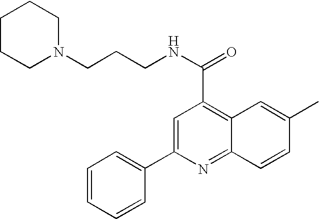 | D |
| 45 | 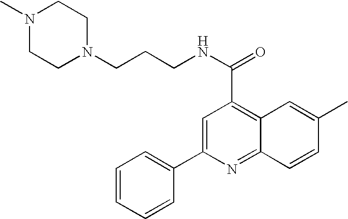 | D |
| 46 | 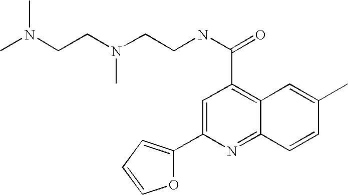 | D |
| 47 | 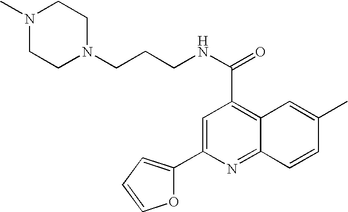 | D |
| 48 | 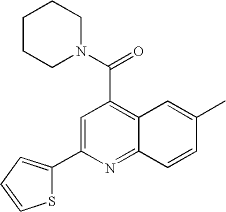 | D |
| 49 | 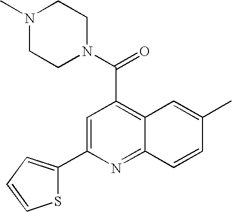 | D |
| 50 | 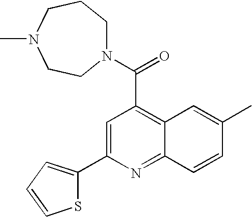 | D |
| 51 | 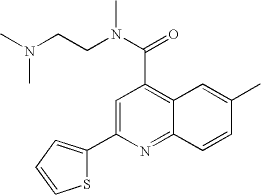 | D |
| 52 | 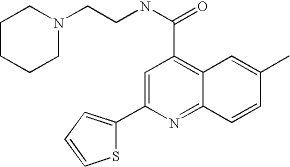 | D |
| 53 | 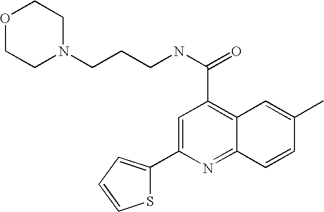 | D |
| 54 | 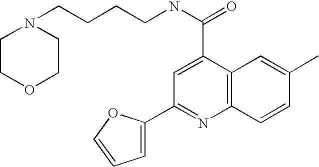 | |
| 55 | 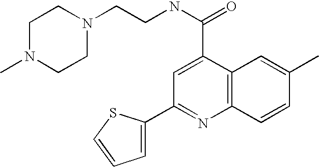 | |
| 56 | 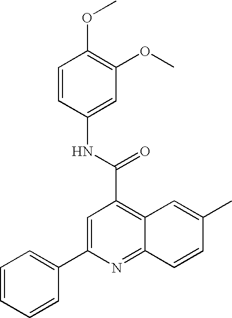 | |
| 57 | 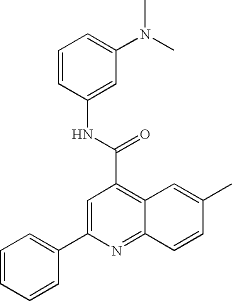 | |
| 58 | 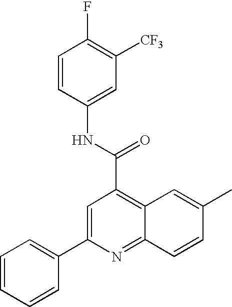 | |
| 59 | 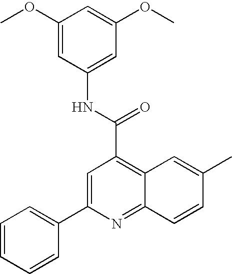 | |
| 60 | 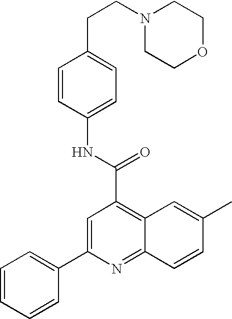 | |
| 61 | 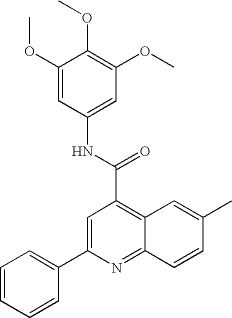 | |
| 62 | 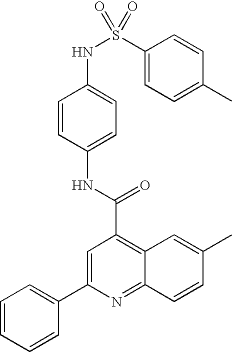 | |
| 63 | 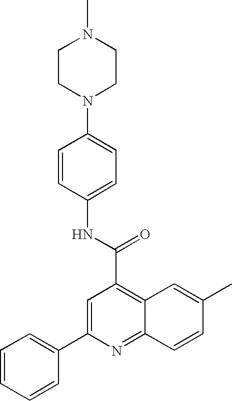 | |
| 64 | 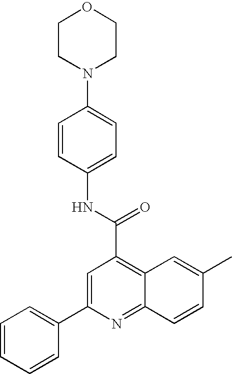 | |
| 65 | 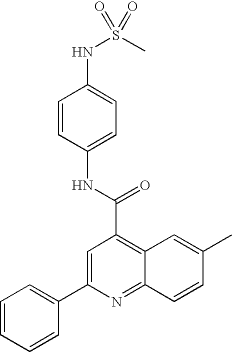 | |
| 66 | 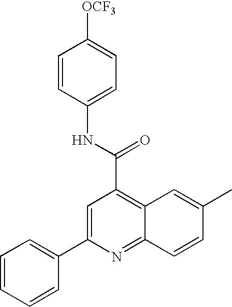 | |
| 67 | 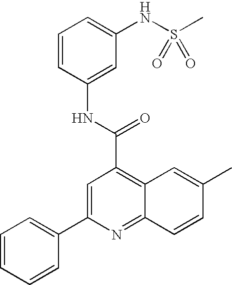 | |
| 68 | 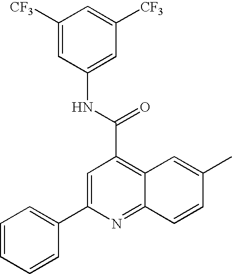 | |
| 69 | 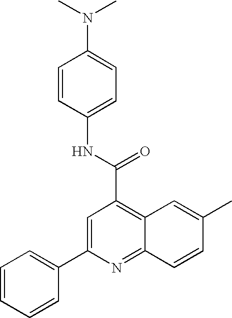 | |
| 70 | 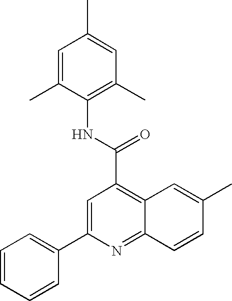 | |
| 71 | 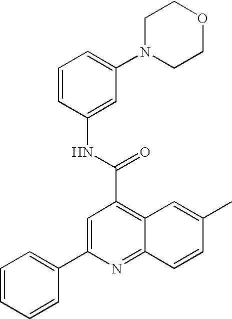 | |
| 72 | 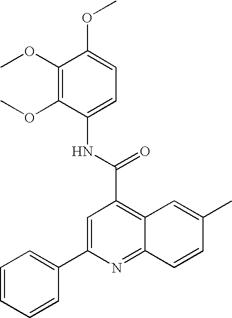 | |
| 73 | 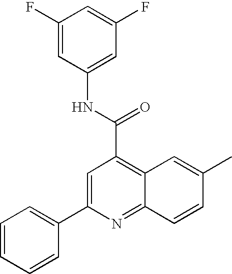 | |
| 74 | 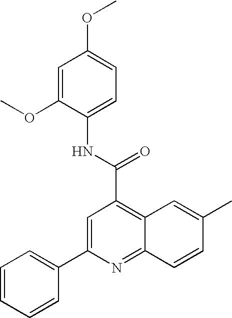 | |
| 75 | 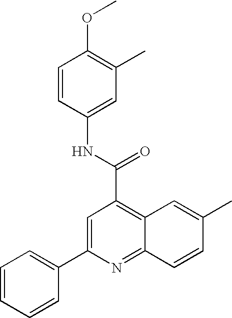 | |
| 76 | 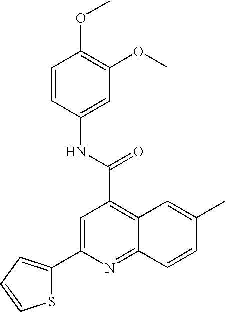 | |
| 77 | 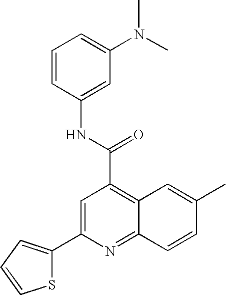 | |
| 78 | 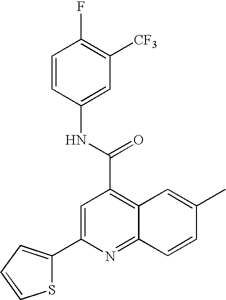 | |
| 79 | 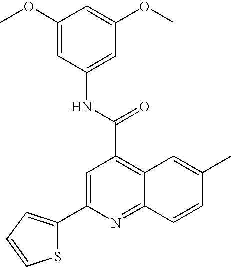 | |
| 80 | 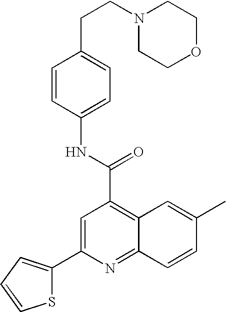 | |
| 81 | 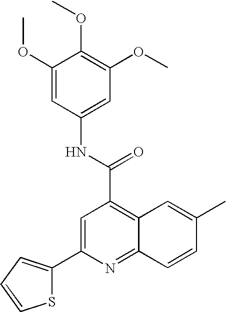 | |
| 82 | 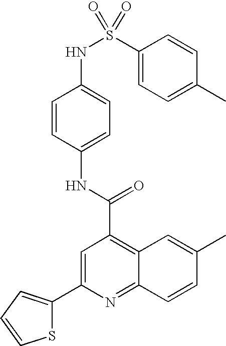 | |
| 83 | 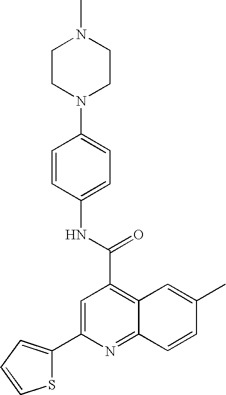 | |
| 84 | 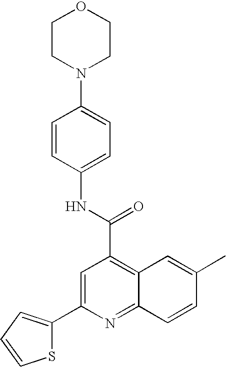 | |
| 85 | 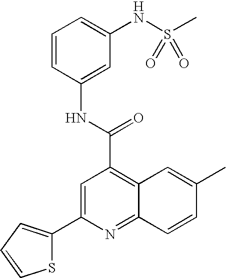 | |
| 86 | 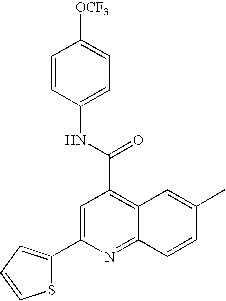 | |
| 87 | 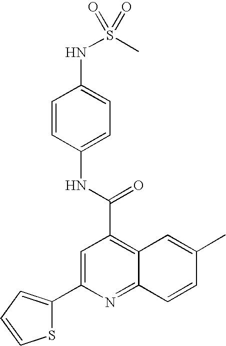 | |
| 88 | 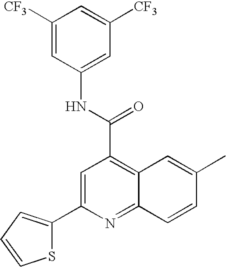 | |
| 89 | 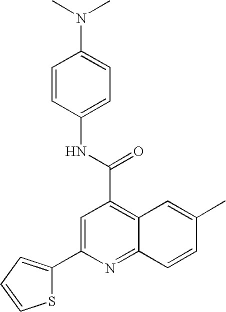 | |
| 90 | 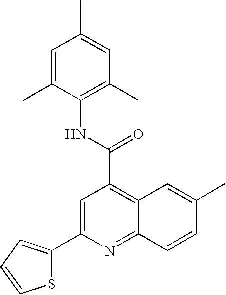 | |
| 91 | 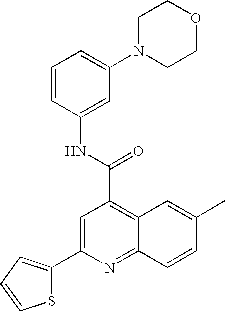 | |
| 92 | 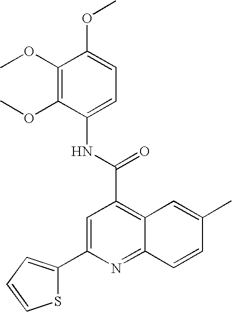 | |
| 93 | 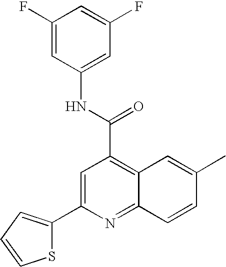 | |
| 94 | 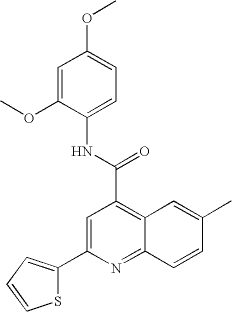 | |
| 95 | 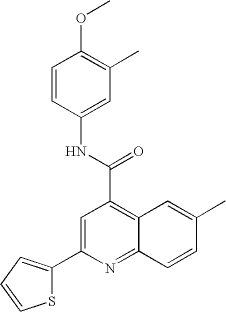 | |
| 96 | 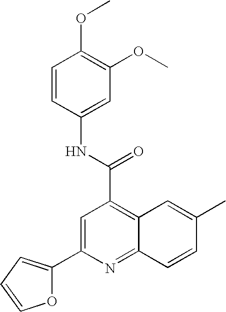 | |
| 97 | 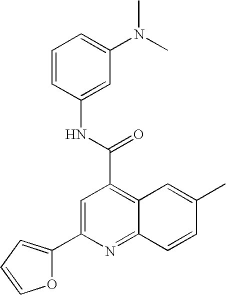 | |
| 98 | 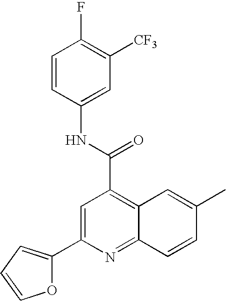 | |
| 99 | 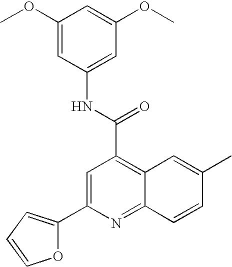 | |
| 100 | 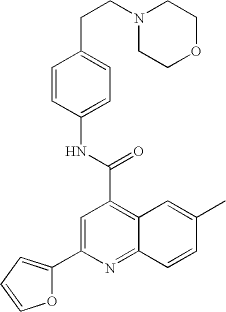 | |
| 101 | 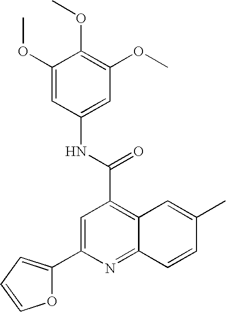 | |
| 102 | 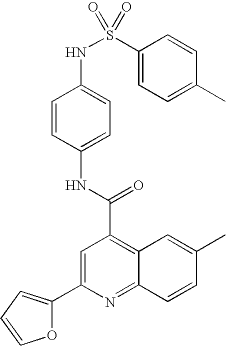 | |
| 103 | 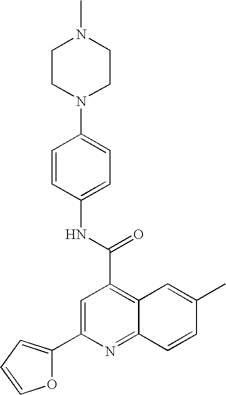 | |
| 104 | 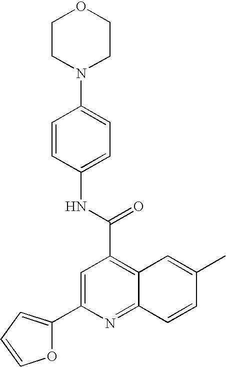 | |
| 105 | 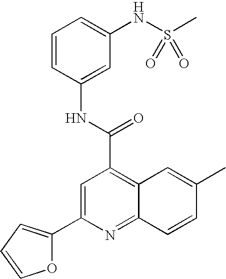 | |
| 106 | 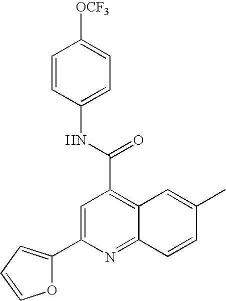 | |
| 107 | 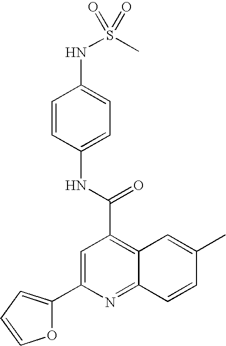 | |
| 108 | 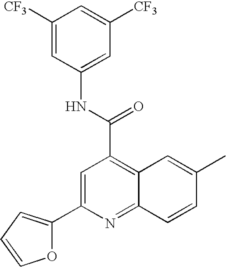 | |
| 109 | 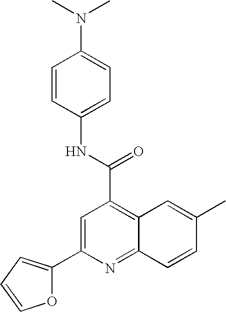 | |
| 110 | 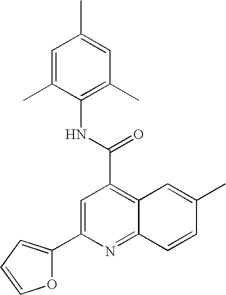 | |
| 111 | 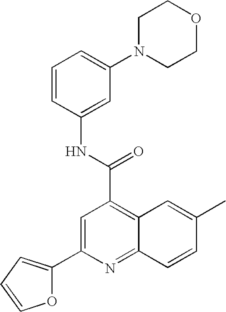 | |
| 112 | 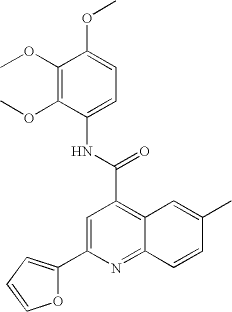 | |
| 113 | 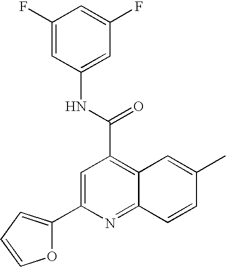 | |
| 114 | 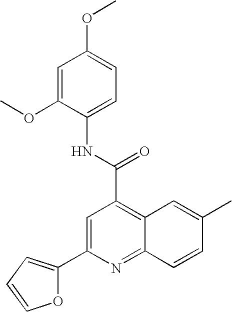 | |
| 115 | 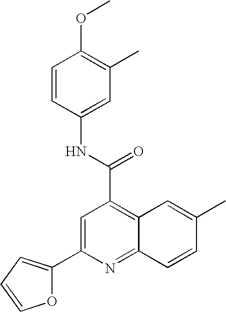 | |
| 116 | 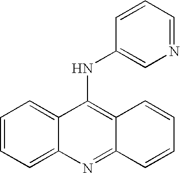 | NT |
| 117 | 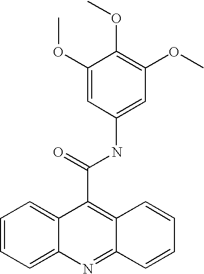 | NT |
| 118 | 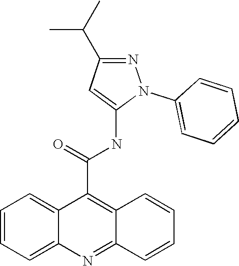 | A |
| 119 | 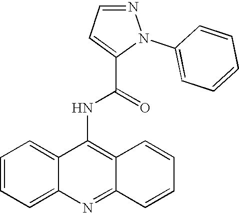 | NT |
| 120 | 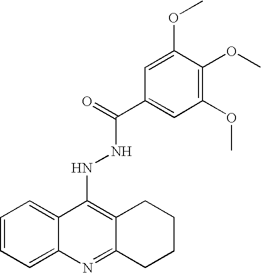 | A |
| 121 | 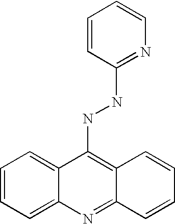 | A |
| 122 | 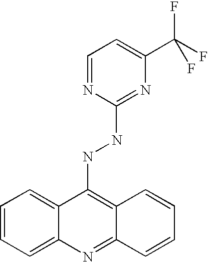 | C |
| 123 | 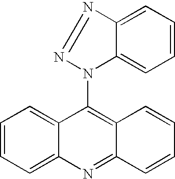 | NT |
| 124 | 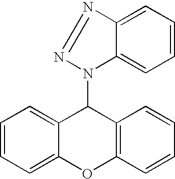 | D |
| 125 | 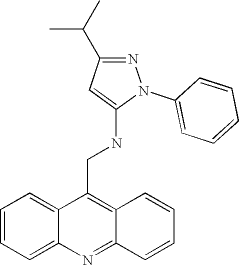 | D |
| 126 | 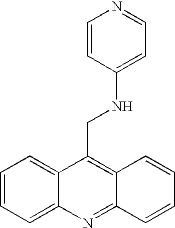 | NT |
| 127 | 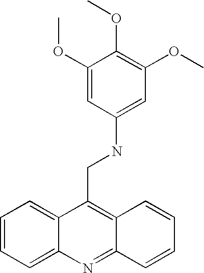 | D |
| 128 | 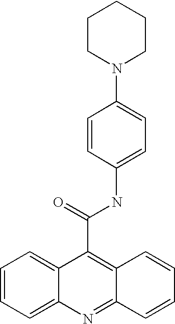 | D |
| 129 | 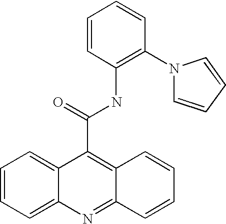 | D |
| 130 | 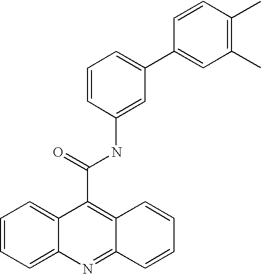 | D |
| 131 | 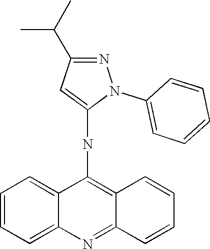 | D |
| 132 | 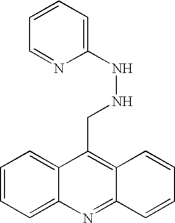 | A |
| 133 | 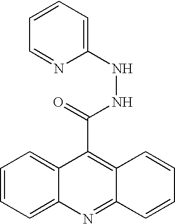 | A |
| 134 | 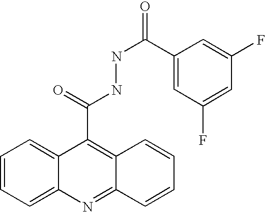 | D |
| 135 | 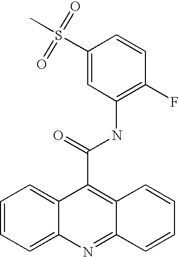 | D |
| 136 | 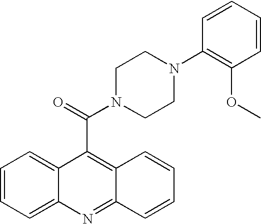 | D |
| 137 | 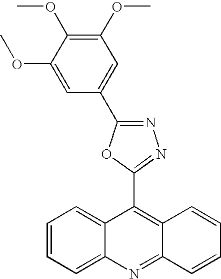 | A |
| 138 | 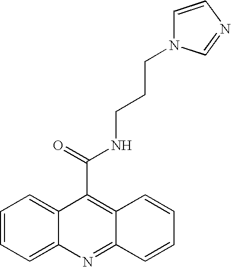 | NT |
| 139 | 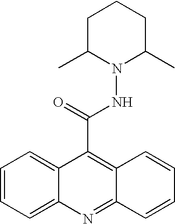 | NT |
| 140 | 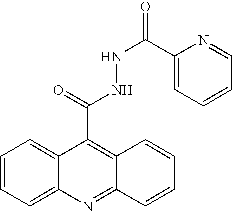 | NT |
| 141 | 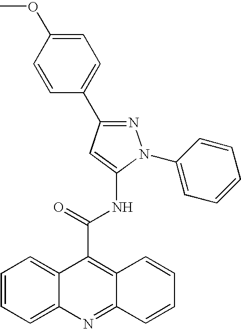 | NT |
| 142 | 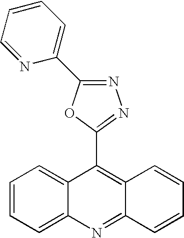 | NT |
| 143 | 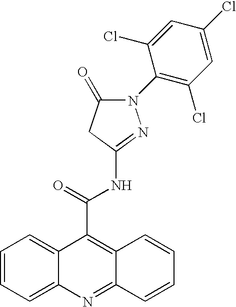 | NT |
| 144 | 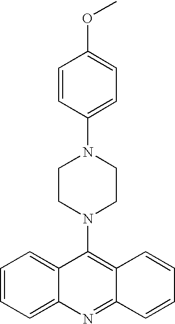 | NT |
| 145 | 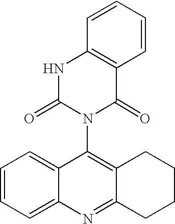 | NT |
| 146 | 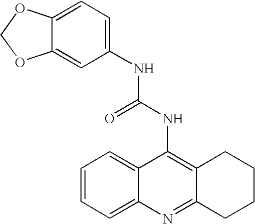 | NT |
| 147 | 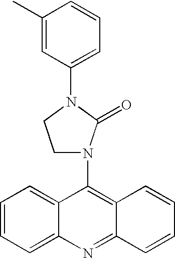 | NT |
| 148 | 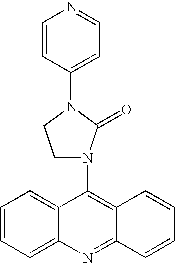 | NT |
| 149 | 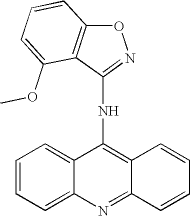 | NT |
| 150 | 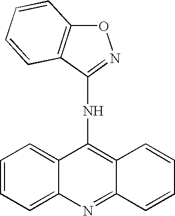 | NT |
| 151 | 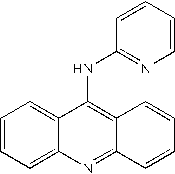 | NT |
| 152 | 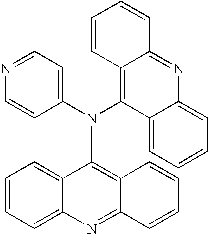 | NT |
| 153 | 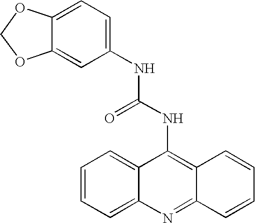 | D |
| 154 | 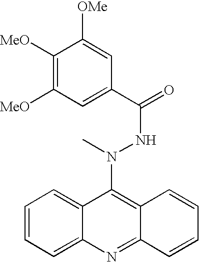 | D |
| 155 | 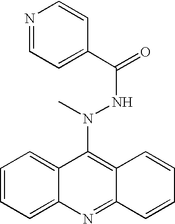 | A |
| 156 | 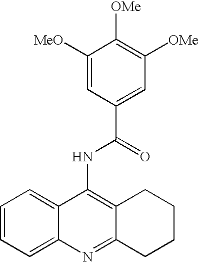 | NT |
| 157 | 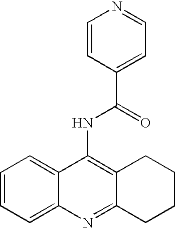 | D |
| 158 | 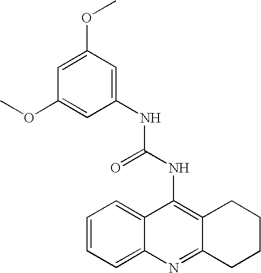 | NT |
| 159 | 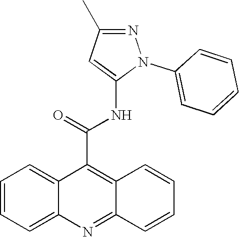 | NT |
| 160 | 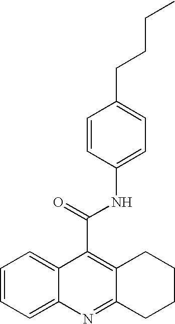 | D |

TABLE 5

| COMPOUND NO | STRUCTURE | IC₅₀ |
|---|---|---|
| 161 | 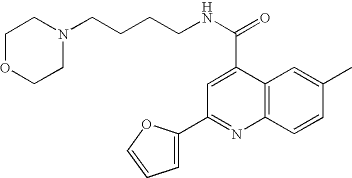 | D |
| 162 | 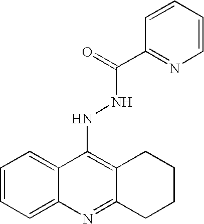 | A |
| 163 | 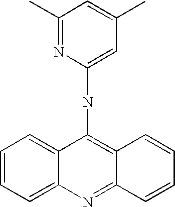 | D |
| 164 | 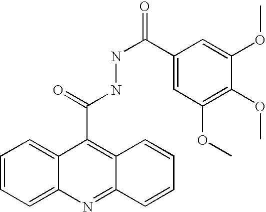 | A |
| 165 | 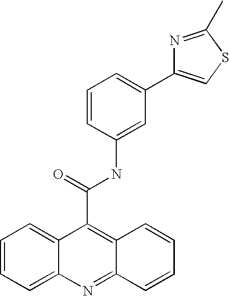 | D |
| 166 | 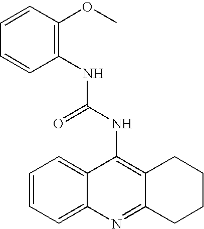 | D |
| 167 | 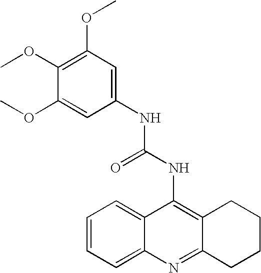 | C |
| 168 | 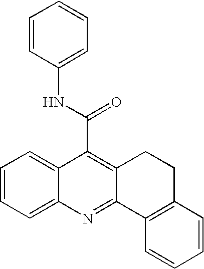 | D |
| 169 | 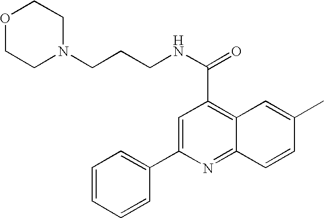 | D |
| 170 | 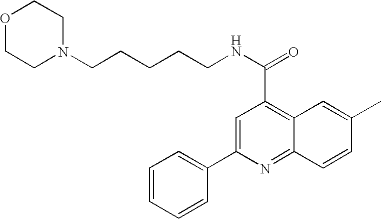 | D |

TABLE 6

| COMPOUND NO | STRUCTURE | ED₅₀ |
|---|---|---|
| 1 | 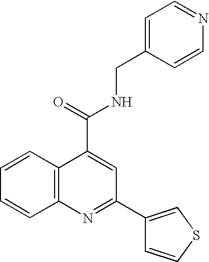 | B |
| 8 | 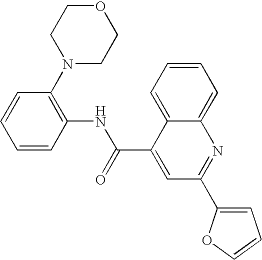 | A |
| 11 | 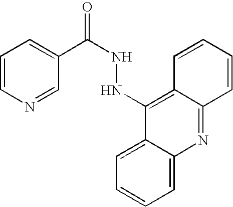 | B |
| 12 | 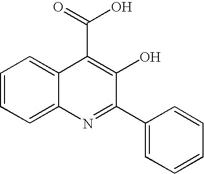 | B |

TABLE 7

| COMPOUND NO | STRUCTURE | IC₅₀ |
|---|---|---|
| 13 | 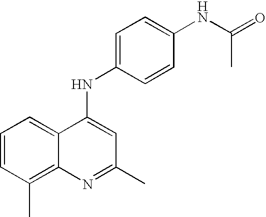 | C |
| 14 | 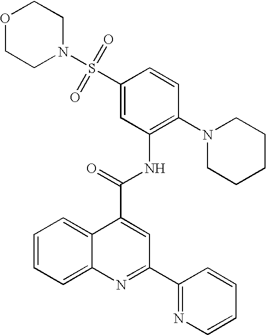 | N/A |

Modulators

In one aspect, the invention provides novel [symbol] modulating compounds for treating and/or preventing a wide variety of diseases and disorders including, for example, diseases or disorders related to aging or stress, diabetes, obesity, neurodegenerative diseases, ocular diseases and disorders, cardiovascular disease, blood clotting disorders, inflammation, cancer, and/or flushing, etc. [symbol] -modulating compounds that increase the level and/or activity of a sirtuin protein may also be used for treating a disease or disorder in a subject that would benefit from increased mitochondrial activity, for enhancing muscle performance, for increasing muscle ATP levels, or for treating or preventing muscle tissue damage associated with hypoxia or ischemia. Other compounds disclosed herein may be suitable for use in a pharmaceutical composition and/or one or more methods disclosed herein.

In one embodiment, [symbol] -modulating compounds of the invention are represented by Structural Formula (I):

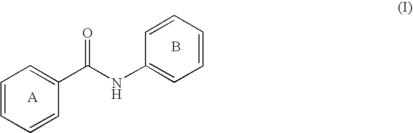

or a salt thereof, where:

Ring A is optionally substituted; and

Ring B is substituted with at least one carboxy or polycyclic aryl group.

In another embodiment, [symbol] -modulating compounds of the invention are represented by Structural Formula (II):

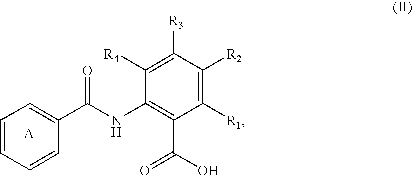

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙORₛ, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

In certain embodiments, R₁, R₂, R₃ and R₄ are independently selected from the group consisting of —H, —OR₅ and —SR₅, particularly —H and —OR₅ (e.g., —H, —OH, —OCH₃).

Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

In yet another aspect, the invention provides novel [symbol] -modulating compounds of Formula (III):

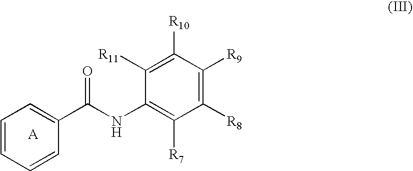

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)NR₅R₆, —NR₅R₆, —NR₅C(O)OR₆, —NR₅C(O)R₆ and —NO₂;

R₈ is a polycyclic aryl group; and

n is 1 or 2.

In certain embodiments, one or more of R₇, R₉, R₁₀ and R₁₁ are —H. In particular embodiments, R₇, R₉, R₁₀ and R₁₁ are each —H.

In certain embodiments, R₅ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle.

In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of said additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuranyl, indolyl, quinoxalinyl, benzothiazolyl, benzooxazolyl, benzimidazolyl, quinolinyl, isoquinolinyl or isoindolyl. In certain such embodiments, R₈ is selected from thiazolopyridyl, imidazothiazolyl, benzooxazinonyl, or imidazopyridyl.

Particular examples of R₈, where

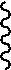

indicates attachment to the remainder of Structural Formula (III), include:

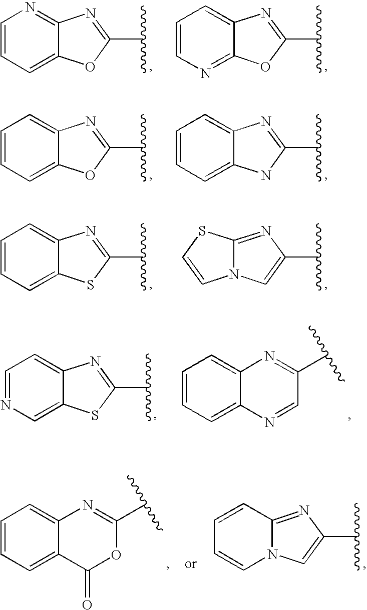

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

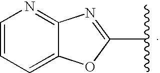

In certain embodiments, R₈ is

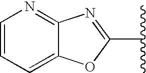

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

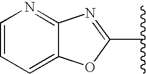

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

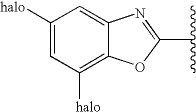

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

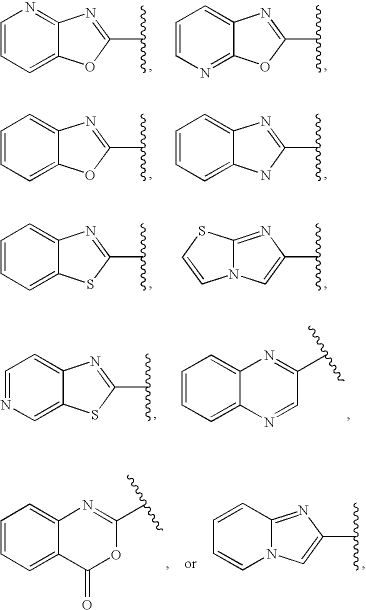

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

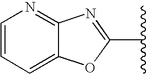

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

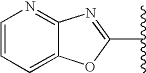

and Ring A is 3,4 dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ or R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

In certain embodiments, the compounds of the invention exclude Compounds I-18 and/or Compounds 116-132.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.
[symbol] -modulating compounds of the invention having hydroxyl substituents, unless otherwise indicated, also include the related secondary metabolites, particularly sulfate, phosphate, acyl (e.g., acetyl, fatty acid acyl) and sugar (e.g., glucuronate, glucose) derivatives. In other words, substituent groups —OH also include —OSO₃⁻ M₊, where M⁺ is a suitable cation (preferably H⁺, NH₄⁺ or an alkali metal ion such as Na⁺ or K⁺) and sugars such as

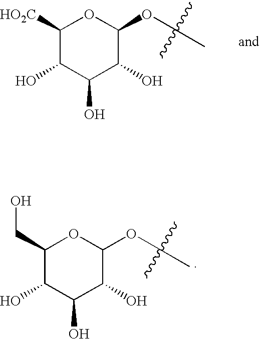

These groups are generally cleavable to —OH by hydrolysis or by metabolic (e.g., enzymatic) cleavage.

[symbol] modulating compounds of the invention, advantageously [symbol] modulate the level and/or activity of a protein.

Separately or in addition to the above properties, certain [symbol] modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8.

Examples of straight chained and branched alkyl groups include methyl, ethyl, n-, propyl, iso-propyl, n-butyl, sec-butyl, tert-butyl, pentyl, hexyl, pentyl and octyl. A C1-C4 straight chained or branched alkyl group is also referred to as a “lower alkyl” group.

TABLE 4

| | | | | FOLD |
|---|---|---|---|---|
| COMPOUND | | ED₅₀ | ED₅₀ | ACTIVATION |
| NO | STRUCTURE | FP | MS | MS |
| 1 | 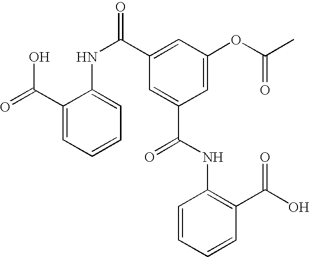 | A | | |
| 2 | 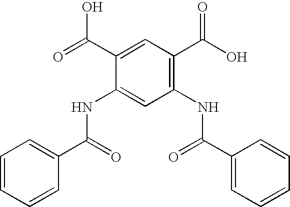 | D | | |
| 3 | 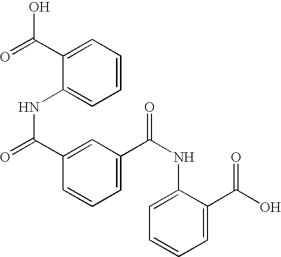 | B | | |
| 4 | 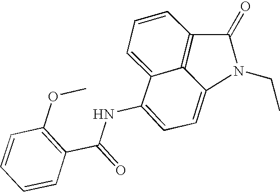 | N/A | | |
| 5 | 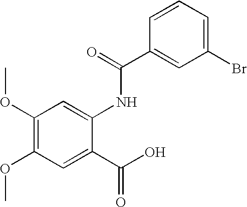 | B | | |
| 6 | 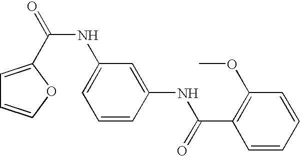 | D | | |
| 7 | 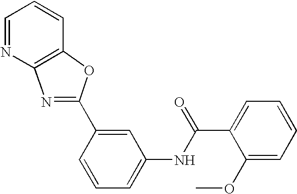 | A | | |
| 8 | 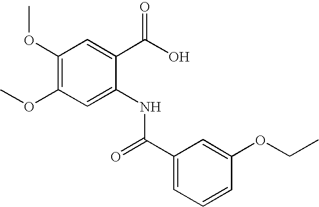 | B | | |
| 9 | 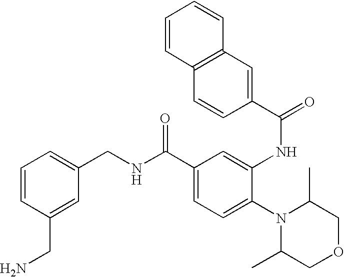 | C | | |
| 10 | 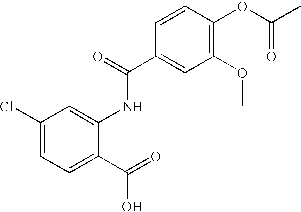 | D | | |
| 52 | 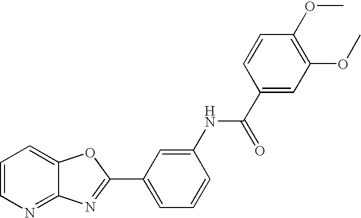 | A | A | B |
| 54 | 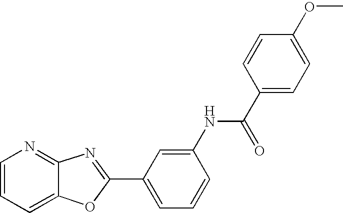 | NT | D | |
| 40 | 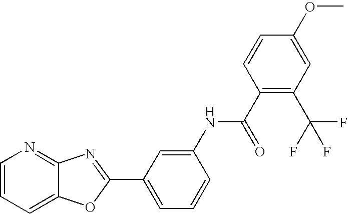 | D | D | C |
| 41 | 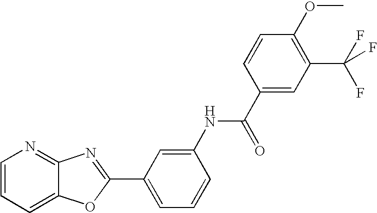 | D | D | C |
| 48 | 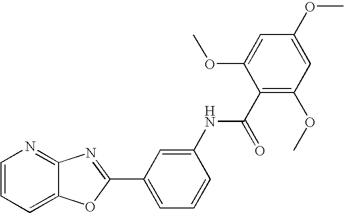 | D | D | C |
| 42 | 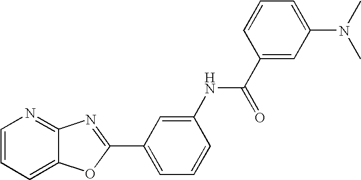 | A | A | B |
| 49 | 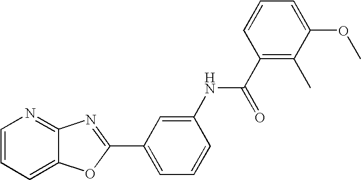 | D | A | B |
| 56 | 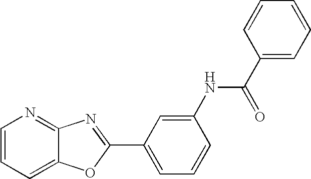 | D | A | B |
| 43 | 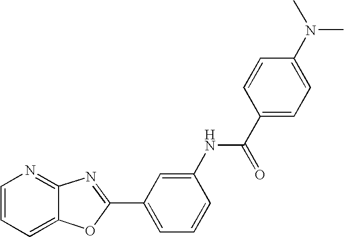 | A | A | B |
| 50 | 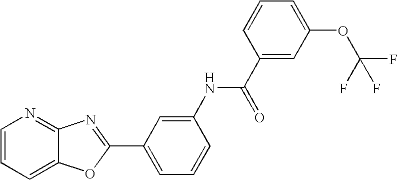 | NT | D | |
| 55 | 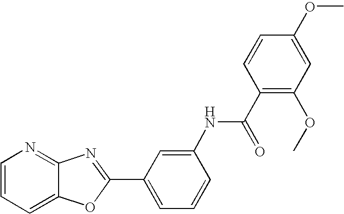 | A | A | B |
| 51 | 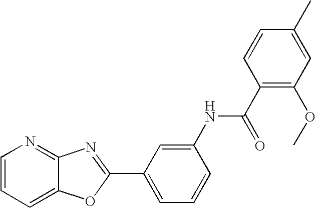 | A | A | B |
| 38 | 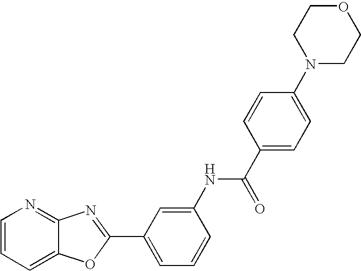 | D | D | C |
| 45 | 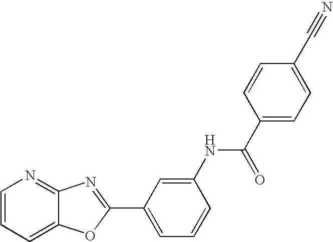 | D | D | C |
| 39 | 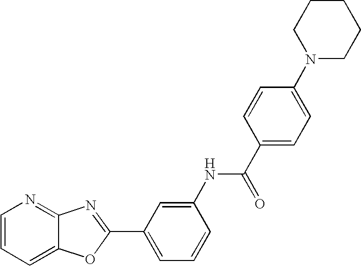 | D | D | C |
| 46 | 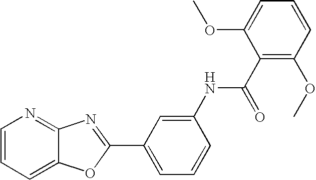 | D | D | C |
| 53 | 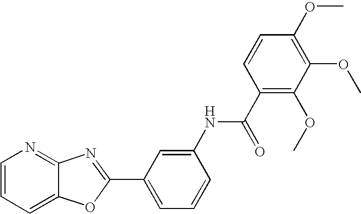 | D | D | C |
| 109 | 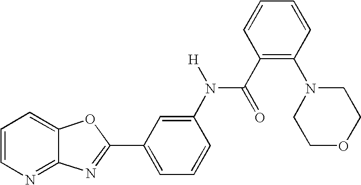 | | A | B |
| 110 | 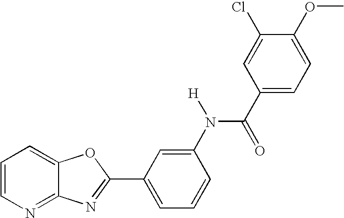 | | C | |
| 111 | 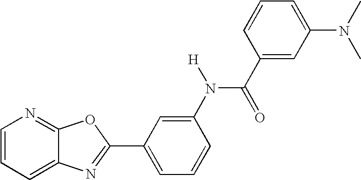 | | A | B |
| 112 | 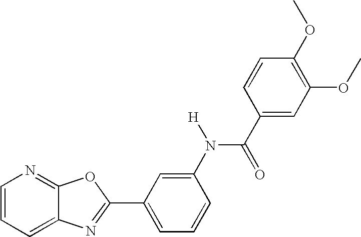 | | A | B |
| 113 | 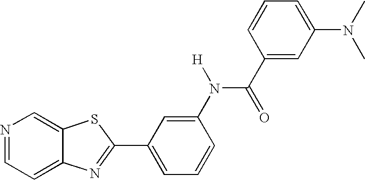 | | A | B |
| 114 | 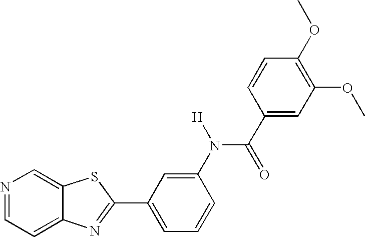 | | A | A |
| 115 | 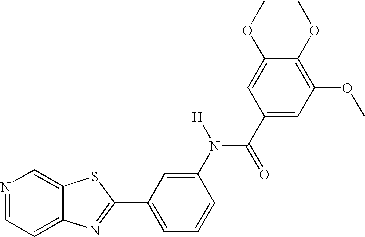 | | A | A |
| 116 | 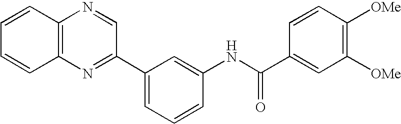 | | C | |
| 117 | 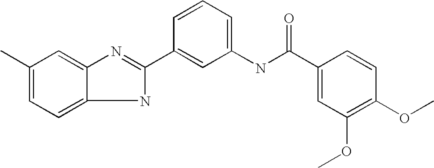 | | A | A |
| 118 | 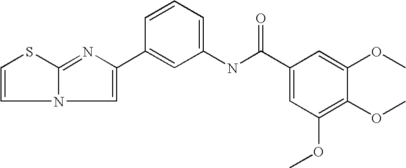 | | A | A |
| 119 | 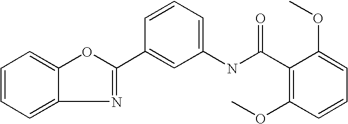 | | A | B |
| 120 | 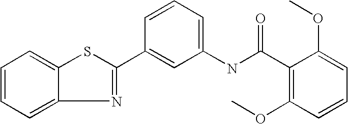 | | NT | |
| 121 | 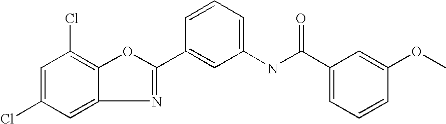 | | D | C |
| 122 | 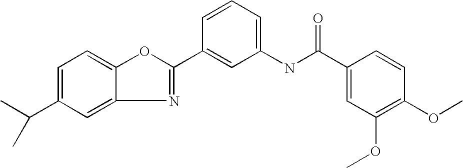 | | C | |
| 123 | 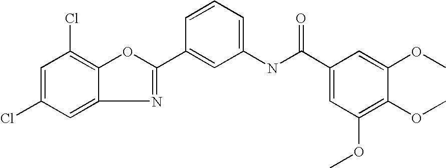 | | NT | |
| 124 | 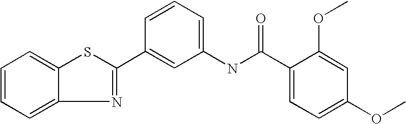 | | A | B |
| 125 | 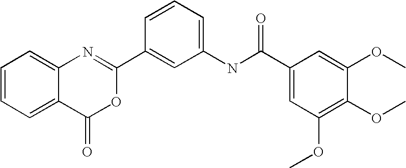 | | B | B |
| 126 | 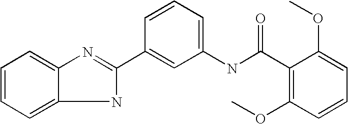 | | A | B |
| 127 | 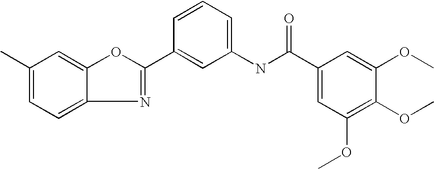 | | A | A |
| 128 | 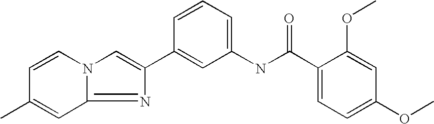 | | A | B |
| 129 | 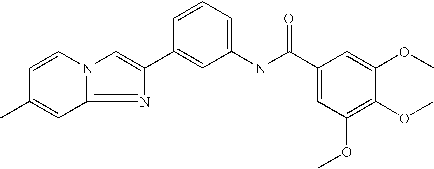 | | A | A |
| 130 | 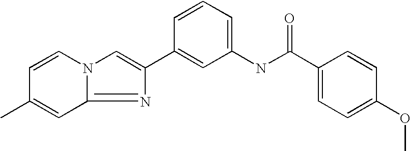 | | A | B |
| 131 | 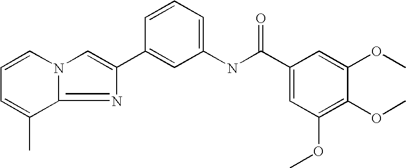 | | A | A |
| 132 | 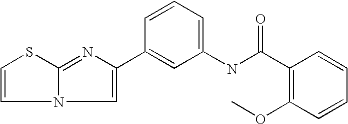 | | A | A |
| 133 | 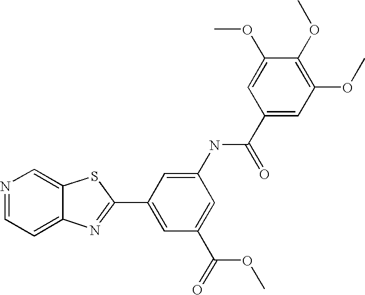 | | A | B |
| 134 | 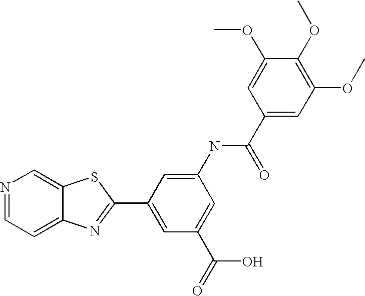 | | A | B |
| 135 | 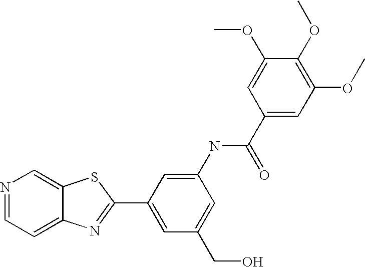 | | A | B |
| 136 | 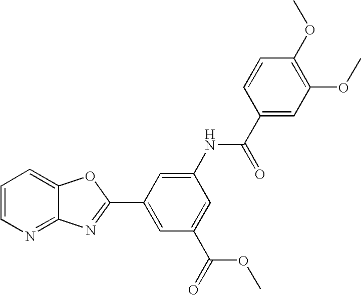 | | | |
| 137 | 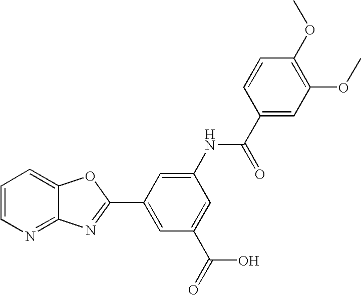 | | D | |
| 164 | 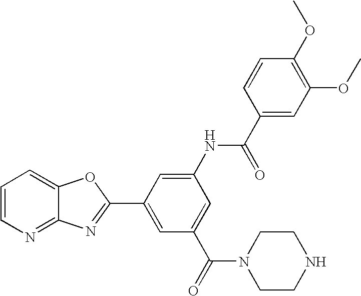 | | A | B |
| 138 | 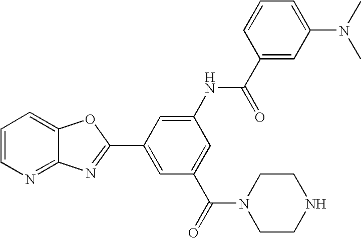 | | A | B |
| 139 | 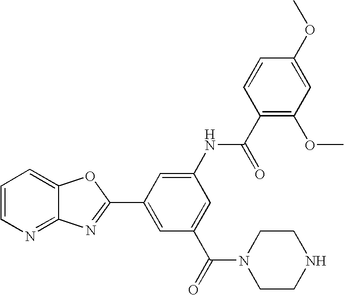 | | A | B |
| 153 | 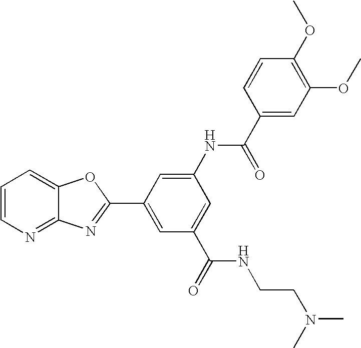 | | | |
| 154 | 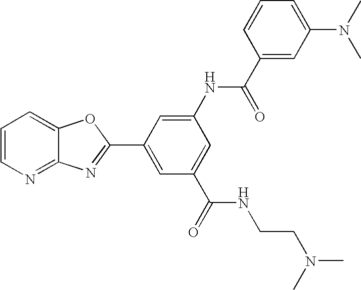 | | | |
| 155 | 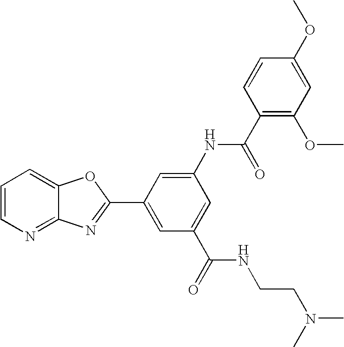 | | | |

TABLE 5

| COM- | | |
|---|---|---|
| POUND | | |
| NO | STRUCTURE | IC₅₀ |
| 13 | 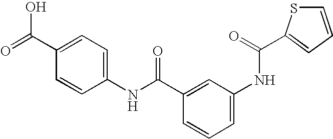 | A |

TABLE 6

| COMPOUND NO | STRUCTURE | ED₅₀ |
|---|---|---|
| 11 | 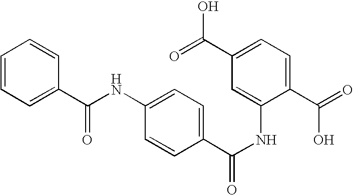 | N/A |
| 12 | 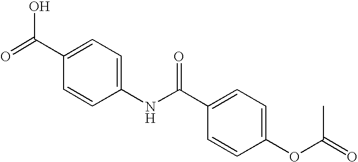 | N/A |

In one embodiment, [symbol] modulating compound represented by Structural Formula (I):

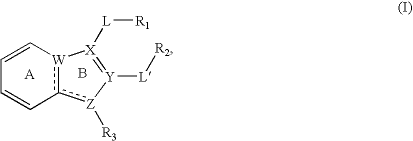

or a salt thereof where, as valence permits:

Ring A is optionally substituted;

L is absent, substituted or unsubstituted phenylene, substituted or unsubstituted —O-phenylene, substituted or unsubstituted thienylene, substituted or unsubstituted pyrazolylene, substituted or unsubstituted benzothiazolylene, —NR₄—, —C(O)O—, —C(O)NR₄—, —NR₄C(O)—, —NR₄—C(O)—NR₅—, —S—, —CHR₆═CHR₇— or —CHR₆—C(O)—;

L′ is absent, substituted or unsubstituted phenylene, substituted or unsubstituted —O-phenylene, substituted or unsubstituted thienylene, substituted or unsubstituted pyrazolylene, substituted or unsubstituted benzothiazolylene, substituted or unsubstituted indenedionylene, —C(O)O—, —C(O)NR₄—, —NR₄C(O)—, —NR₄—C(O)—NR₅—, —S—, —CHR₆═CHR₇— or —CHR₈—C(O)—, provided that at least one of L and L′ is substituted or unsubstituted phenylene, substituted or unsubstituted —O-phenylene, substituted or unsubstituted thienylene, substituted or unsubstituted pyrazolylene, substituted or unsubstituted benzothiazolylene, substituted or unsubstituted indenedionylene, —NR₄—, —C(O)O—, —C(O)NR₄—, —NR₄C(O)—, —NR₄—C(O)—NR₅—, —S—, —CHR₆═CHR₇— or —CHR₈—C(O)—;

R₁ is absent, —H, —NR₄R₅, —N₄C(O)R₅, —OR₅, naphthyl or a heterocyclic group, provided that L and R₁ are not both absent unless X is N;

R₂ is —H, unsubstituted alkyl, —NR₄R₅, —NR_{4l C(O)R}₅, —OR₅, substituted or unsubstituted phenyl, naphthyl or a heterocyclic group;

R₃ is —H, —NR₄R₅, —N₄C(O)R₅, —OR₅ or a substituted or unsubstituted heterocyclic group, or R₂ and R₃, taken together with the atoms to which they are attached, form an optionally substituted heterocyclic group, or R₃ is absent when Z is O or S;

R₄ and R₅ are independently —H, a substituted or unsubstituted alkyl group, a substituted or unsubstituted aryl group or a substituted or unsubstituted non-aromatic heterocyclic group;

R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)R₄, —S(O)OR₄, —S(O)NR₄R₅, —NR₄R₅, —NR₄C(O)OR₅, —NR₄C(O)R₅ and —NO₂;

W is C or N;

X is C or N;

Y is C or N;

Z is C, N, O or S, provided that at least two of W, X, Y and Z are C; and

n is 1 or 2.

In a second embodiment, [symbol] modulating compounds of the invention are represented by Structural Formula (Ia):

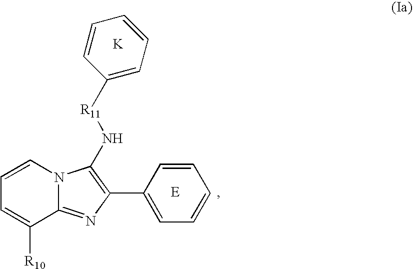

wherein:

R₁₀ is selected from —H, —C(O)—N(R₄₀)(R₅₀), —S(O)₂N(R₄₀)(R₅₀), or —CH₂—N(R₄₀)R₅₀); wherein each of R₄₀ and R₅₀ is independently selected from —H, —C₁-C₃ straight or branched alkyl, —(C₁-C₃ straight or branched alkyl)-N(CH₃)₂, —(C₁-C₃ straight or branched alkyl)-heterocyclyl, —(C₁-C₃ straight or branched alkyl)-alkylheterocyclyl, or wherein R₄₀ and R₅₀ taken together with the N atom to which they are bound form a 5-6 membered heterocyclic ring that is optionally substituted with —(C₁-C₃ straight or branched alkyl), and wherein at least one of R₄₀ or R₅₀ is not H;

R₁₁ is selected from —C₁-C₃ straight or branched alkylene or —C(O)—; and

each of ring K and ring E is independently substituted with up to three substituents independently selected from halo, —CF₃, —O—(C₁-C₃ straight or branched alkyl), —S—(C₁-C₃ straight or branched alkyl), —N(R₄₀)(R₅₀), —S(O)₂—N(R₄₀)(R₅₀), heterocyclyl, (C₁-C₃ straight or branched alkyl)-heterocyclyl, —O—(C₁-C₃ straight or branched alkyl)-heterocyclyl, —S—(C₁-C₃ straight or branched alkyl)-heterocyclyl, or is optionally fused to a 5-6 membered heterocyclyl or heteroaryl, wherein any heterocyclcyl or heteroaryl is optionally substituted with —C₁-C₃ straight or branched alkyl.

In certain embodiments, one of R₄₀ or R₅₀ is H.

In certain embodiments, ring K is substituted with up to 3 substituents independently selected from methyl, —O-methyl, —N(CH₃)₂, or —CF₃, but is unsubstituted in the positions ortho to the attachment to the rest of the molecule.

In certain embodiments, such as where R₄₀ and/or R₅₀ have the values indicated above and/or ring K has the substitution pattern described above, ring E is substituted with up to 2 substituents independently selected from methyl, —O-methyl, —S(O)₂—N(CH₃)₂, —O-methyl-morpholino, —O-ethyl-morpholino, fluoro, —CF₃, piperidyl, methylpiperidyl, pyrrolidyl, or methylpyrrolidyl.

In certain embodiments, R₁₀ is selected from —H, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl. —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2.

In particular embodiments, ring K is substituted with up to 3 substituents independently selected from methyl, O-methyl, N(CH₃)₂, CF₃, but is unsubstituted in the positions ortho to the attachment to the rest of the molecule; ring E is substituted with up to 2 substituents independently selected from methyl, O-methyl, —S(O)₂—N(CH₃)₂, —O-methyl-morpholino, —O-ethyl-morpholino, fluoro, —CF₃, methylpiperidyl, or pyrrolidyl; and R₁₀ is selected from —H, —CH₂-piperazinyl, —C(O)—NH—(CH₂)₂-piperazinyl, —C(O)—NH—(CH₂)₂-methylpiperazinyl, —C(O)—NH—(CH₂)₂-pyrrolidyl, or —C(O)—NH—(CH₂)₂—N(CH₃)₂.

In a further embodiment, [symbol] -modulating compounds of the invention are represented by Formula (Ib)):

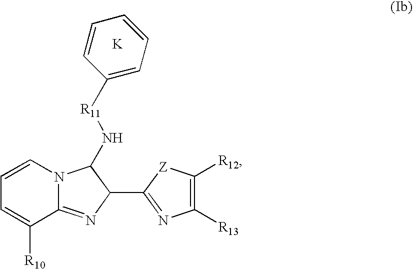

wherein:

Z is selected from O or S;

R₁₀ is selected from —H, —C(O)—N(R₄₀)(R₅₀), —S(O)₂N(R₄₀)(R₅₀), or —CH₂—N(R₄₀)(R₅₀); wherein each of R₄₀ and R₅₀ is independently selected from —H, —C₁-C₃ straight or branched alkyl, —(C₁-C₃ straight or branched alkyl)-N(CH₃)₂, —(C₁-C₃ straight or branched alkyl)-heterocyclyl, —(C₁-C₃ straight or branched alkyl)-alkylheterocyclyl, or wherein R₄₀ and R₅₀ taken together with the N atom to which they are bound form a 5-6 membered heterocyclic ring that is optionally substituted with —(C₁-C₃ straight or branched alkyl), and wherein at least one of R₄₀ or R₅₀ is not IL

R₁₁ is selected from —C₁-C₃ straight or branched alkylene or —C(O)—;

each of R₁₂ and R₁₃ is independently selected from —H or —(C₁-C₃ straight or branched alkyl), or R₁₂ and R₁₃ are taken together to form a benzene ring that is substituted with up to two substituents independently selected from —(C₁-C₃ straight or branched alkyl), —CF₃ or halo; and

ring K is substituted with up to three substituents independently selected from halo, —CF₃, —O—(C₁-C₃ straight or branched alkyl), —S—(C₁-C₃ straight or branched alkyl), —N(R₄₀)(R₅₀), —S(O)₂—N(R₄₀)(R₅₀), heterocyclyl, (C₁-C₃ straight or branched alkyl)-heterocyclyl, —O—(C₁-C₃ straight or branched alkyl)-heterocyclyl, —S—(C₁-C₃ straight or branched alkyl)-heterocyclyl, or is optionally fused to a 5-6 membered heterocyclyl or heteroaryl, wherein any heterocyclcyl or heteroaryl is optionally substituted with —C₁-C₃ straight or branched alkyl.

In certain embodiments, R₁₀ is —H.

In certain embodiments, ring K is substituted with up to 3 substituents independently selected from methyl, O-methyl, N(CH₃)₂, CF₃, and wherein ring K is unsubstituted in the positions ortho to the attachment to the rest of the molecule.

In certain embodiments, such as when R₁₀ is —H and/or ring K has the substitution pattern described above, each of R₁₂ and R₁₃ is independently selected from —H, methyl, —O-methyl, S(O)₂—N(CH₃)₂, —O-methyl-morpholino, —O-ethyl-morpholino, fluoro, —CF₃, piperidyl, methylpiperidyl, pyrrolidyl, or methylpyrrolidyl.

In a preferred embodiment, each of R₁₂ and R₁₃ is methyl.

In another embodiment, [symbol] -modulating compounds of the invention are represented by Formula (II):

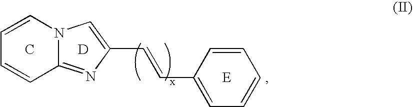

or a salt thereof, where:

Rings C, D and E are optionally substituted; and

x is 0 or 1.

In certain embodiments x is 0.

In certain embodiments (e.g., where x is 0), Ring C is substituted with a group that is capable of providing a trans configuration (e.g., an amide group, an optionally 2-substituted 1-alkenyl group).

One group of compounds of the invention encompassed by Structural Formula (II) is represented by Structural Formula (III):

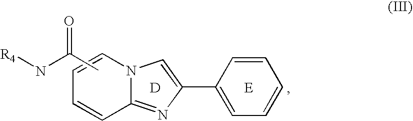

or a salt thereof, where:

Rings D and E are optionally substituted; and

R₄ is —H, a substituted or unsubstituted alkyl group, a substituted or unsubstituted aryl group or a substituted or unsubstituted non-aromatic heterocyclic group.

In certain embodiments, Ring E is substituted with an acylamino, heterocyclylcarbonylamino, lower alkyl or substituted or unsubstituted alkoxy group.

In certain embodiments, Ring D is substituted with an amino group. In particular embodiments, Ring E is substituted with an acylamino, heterocyclylcarbonylamino, lower alkyl, or substituted or unsubstituted alkoxy group, and Ring D is substituted with an amino group.

In certain embodiments, R₄ is a substituted alkyl group.

A group of compounds of the invention encompassed by Structural Formula (III) is represented by Structural Formula (IV):

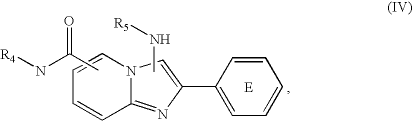

or a salt thereof where:

Ring E is optionally substituted; and

R₄ and R₅ are independently —H, a substituted or unsubstituted alkyl group, a substituted or unsubstituted aryl group or a substituted or unsubstituted non-aromatic heterocyclic group.

In certain embodiments, Ring E is substituted with an acylamino, heterocyclylcarbonylamino, lower alkyl or substituted or unsubstituted alkoxy group.

In certain embodiments, R₄ is a substituted alkyl group. In particular embodiments, Ring E is substituted with an acylamino, heterocyclylcarbonylamino, lower alkyl or substituted or unsubstituted alkoxy group and R₄ is a substituted alkyl group.

In certain embodiments, R₅ is a substituted or unsubstituted alkyl group, such as an aralkyl or a cycloalkyl group (e.g., benzyl, cyclohexyl). In particular embodiments, R₅ is a substituted or unsubstituted alkyl group when Ring E is substituted with an acylamino, heterocyclylcarbonylamino, lower alkyl or substituted or unsubstituted alkoxy group and/or R₄ is a substituted alkyl group.

One group of compounds of the invention encompassed by Structural Formula (IV) is represented by Structural Formula (IVa):

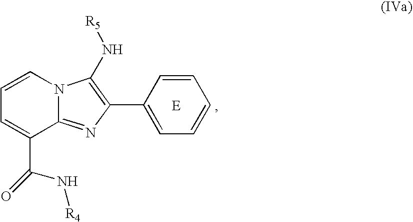

or a salt thereof.

Another group of compounds of the invention encompassed by Structural Formula (IV) is represented by Structural Formula (IVb):

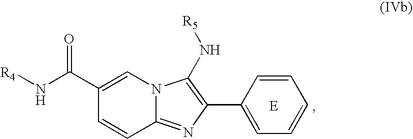

or a salt thereof.

Yet another group of compounds of the invention encompassed by Structural Formula (IV) is represented by Structural Formula (V):

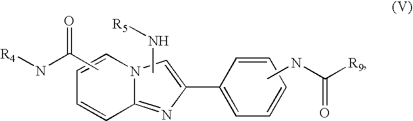

or a salt thereof, where:

R₄, R₅ and R₉ are independently —H, a substituted or unsubstituted alkyl group, a substituted or unsubstituted aryl group or a substituted or unsubstituted non-aromatic heterocyclic group.

In certain embodiments, R₄ is a substituted alkyl group.

In certain embodiments, R₅ is a substituted or unsubstituted alkyl group, such as an aralkyl or a cycloalkyl group (e.g., benzyl, cyclohexyl). In particular embodiments, R₅ is a substituted or unsubstituted alkyl group and R₄ is a substituted alkyl group.

In certain embodiments, R₉ is a C₁₋₄ alkyl group (e.g., methyl, cyclopropyl), a substituted or unsubstituted aryl group (e.g., substituted or unsubstituted phenyl) or a substituted or unsubstituted non-aromatic heterocyclic group (e.g., furanyl, morpholino). In particular embodiments, R₉ is a C₁₋₄ alkyl group, a substituted or unsubstituted aryl group or a substituted or unsubstituted non-aromatic heterocyclic group when R₅ is a substituted or unsubstituted alkyl group and/or R₄ is a substituted alkyl group

A group of compounds of the invention encompassed by Structural Formula (V) are represented by Structural Formula (VI):

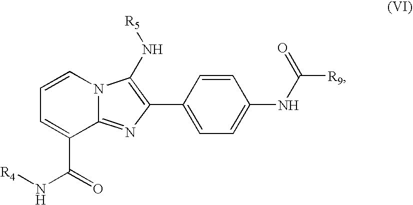

or a salt thereof, where:

R₄, R₅ and R₉ are independently —H, a substituted or unsubstituted alkyl group, a substituted or unsubstituted aryl group or a substituted or unsubstituted non-aromatic heterocyclic group.

In certain embodiments, R₄ is a substituted alkyl group.

In certain embodiments, R₅ is a substituted or unsubstituted alkyl group, such as an aralkyl or a cycloalkyl group (e.g., benzyl, cyclohexyl). In particular embodiments, R₅ is a substituted or unsubstituted alkyl group and R₄ is a substituted alkyl group.

In certain embodiments, R₉ is a C₁₋₄ alkyl group (e.g., methyl, cyclopropyl), a substituted or unsubstituted aryl group (e.g., substituted or unsubstituted phenyl) or a substituted or unsubstituted non-aromatic heterocyclic group (e.g., furanyl, morpholino). In particular embodiments, R₉ is a C₁₋₄ alkyl group, a substituted or unsubstituted aryl group or a substituted or unsubstituted non-aromatic heterocyclic group when R₅ is a substituted or unsubstituted alkyl group and/or R₄ is a substituted alkyl group

In yet another embodiment, [symbol] modulating compounds of the invention are represented by Formula (VII):

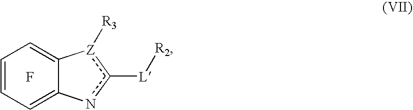

or a salt thereof, where:

Ring F is optionally substituted;

L′ is substituted or unsubstituted phenylene, substituted or unsubstituted thienylene, substituted or unsubstituted indonedionylene, —C(O)O—, —NR₄C(O)—, —S—, —CHR₆═CHR₇— or —CHR₈—C(O)—;

R₂ is —H, unsubstituted alkyl, —NR₄R₅, —NR₄C(O)R₅, —OR₅, substituted or unsubstituted phenyl or a heterocyclic group;

R₃ is —H, —NR₄R₅, —N₄C(O)R₅, —OR₅ or a heterocyclic group, or R₂ and R₃, taken together with the atoms to which they are attached, form an optionally substituted heterocyclic group, or R₃ is absent when Z is O or S;

R₄ and R₅ are independently —H, a substituted or unsubstituted alkyl group, a substituted or unsubstituted aryl group or a substituted or unsubstituted non-aromatic heterocyclic group;

R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

Z is C, N, O or S; and

n is 1 or 2.

In Structural Formula (VII), the dashed bond indicated as [symbol] represents a single or double bond. For the compounds represented by Structural Formula (VII), both dashed bonds cannot be double bonds, but preferably one of the dashed bonds is a double bond and the other is a single bond.

One group of compounds encompassed by Structural Formula (VII) are represented by Structural Formula (VIII):

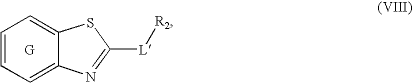

or a salt thereof, where:

Ring G is optionally substituted;

L′ is substituted or unsubstituted phenylene, substituted or unsubstituted —O-phenylene, substituted or unsubstituted thienylene, substituted or unsubstituted indonedionylene, —NR₄C(O)—, —C(O)O—, —S—, —CHR₆═CHR₇ or —CHR₈—C(O)—;

R₂ is —H, unsubstituted alkyl, —NR₄R₅, —NR₄C(O)R₅, —OR₅, substituted or unsubstituted phenyl or a heterocyclic group;

R₄ and R₅ are independently —H, a substituted or unsubstituted alkyl group, a substituted or unsubstituted aryl group or a substituted or unsubstituted non-aromatic heterocyclic group;

R₆, R₇ and R₅ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and

n is 1 or 2.

In certain embodiments, L′ is substituted or unsubstituted —O-phenylene, substituted or unsubstituted thienylene or —CHR₈—C(O)—.

In certain embodiments, R₂ is —NR₄C(O)R₅ or a heterocyclic group, such as —NR₄C(O)-substituted alkyl. In a particular embodiment, R₂ is —NR₄C(O)R₅ or a heterocyclic group and L′ is substituted or unsubstituted —O-phenylene, substituted or unsubstituted thienylene or —CHR₈—C(O)—.

In certain embodiments, Ring G is unsubstituted. In a particular embodiment, Ring G is unsubstituted when R₂ is —NR₄C(O)R₅ or a heterocyclic group and/or L′ is substituted or unsubstituted —O-phenylene, substituted or unsubstituted thienylene or —CHR₈—C(O)—.

In particular embodiments, L′ is substituted or unsubstituted —O-phenylene or —CHR₈—C(O)—. In such embodiments, R₂ is preferably a heterocyclic group.

In particular embodiments, L′ is a substituted or unsubstituted thienylene. In such embodiments, R₂ is —NR₄C(O)R₅.

Another group of compounds of the invention encompassed by Structural Formula (VII) is represented by Structural Formula (IX):

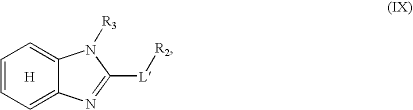

or a salt thereof, wherein:

Ring H is optionally substituted;

L′ is substituted or unsubstituted phenylene, —S— or —CHR₆═CHR₇—;

R₂ is —NR₄R₅, —NR₄C(O)R₅ or a heterocyclic group;

R₃ is —H, or R₂ and R₃, taken together with the atoms to which they are attached, form an optionally substituted heterocyclic group;

R₄ and R₅ are independently —H, a substituted or unsubstituted alkyl group, a substituted or unsubstituted aryl group or a substituted or unsubstituted non-aromatic heterocyclic group;

R₆ and R₇ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and

n is 1 or 2.

In certain embodiments, L′ is —S—. In particular embodiments, L′ is —S— and R₂ and R₃, taken together with the atoms to which they are attached, form an optionally substituted heterocyclic group.

In certain embodiments, L′ is a substituted or unsubstituted phenylene. In particular embodiments, L′ is a substituted or unsubstituted phenylene and R₂ is —NR₄C(O)R₅.

In certain embodiments, L′ is —CHR₆═CHR₇—, such as —CH═CH₂— or —C(CN)═CH₂—. In particular embodiments, L′ is —CHR₆═CHR₇— and R₂ is —NR₄R₅ or a substituted or unsubstituted aryl group.

A further group of compounds of the invention encompassed by Structural Formula (VII) is represented by Structural Formula (X):

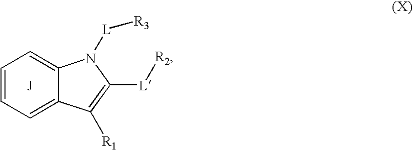

or a salt thereof; where:

Ring J is optionally substituted;

L is substituted or unsubstituted phenylene, substituted or unsubstituted —O-phenylene, substituted or unsubstituted thienylene, substituted or unsubstituted pyrazolylene, substituted or unsubstituted benzothiazolylene, —C(O)O—, —C(O)NR₄—, —NR₄C(O)—, —NR₄—C(O)—NR₅—, —S—, —CHR₄═CHR₇— or —CHR₆—C(O)—;

L′ is substituted or unsubstituted phenylene, substituted or unsubstituted —O-phenylene, substituted or unsubstituted thienylene, substituted or unsubstituted pyrazolylene, substituted or unsubstituted benzothiazolylene, —C(O)O—, —C(O)NR₄—, —NR₄C(O)—, —NR₄—C(O)—NR₅—, —S—, —CHR₆═CHR₇— or —CHR₈—C(O)—;

R₁ is —H, —NR₄R₅, —N₄C(O)R₅, —OR₅, naphthyl or a heterocyclic group;

R₂ is —H, unsubstituted alkyl, —NR₄R₅, —NR₄C(O)R₅, —OR₅, naphthyl or a heterocyclic group;

R₃ is —H, —NR₄R₅, —N₄C(O)R₅, —OR₅ or a substituted or unsubstituted heterocyclic group;

R₄ and R₅ are independently —H, a substituted or unsubstituted alkyl group, a substituted or unsubstituted aryl group or a substituted or unsubstituted non-aromatic heterocyclic group;

R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₒNR₄R₅, —NR₄R₅, —NR₄C(O)OR₅, —NR₄C(O)R₅ and —NO₂; and

n is 1 or 2.

In certain embodiments, L is —NR₄C(O)—.

In certain embodiments, R₃ is a substituted or unsubstituted aryl group. In particular embodiments, R₃ is a substituted or unsubstituted aryl group and L is —NR₄C(O)—.

In certain embodiments, L′ is —C(O)O—. In particular embodiments, L′ is —C(O)O— when R₃ is a substituted or unsubstituted aryl group and/or L is —NR₄C(O)—.

In certain embodiments, R₂ is an unsubstituted alkyl group. In particular embodiments, R₂ is an unsubstituted alkyl group when L′ is —C(O)O—, R₃ is a substituted or unsubstituted aryl group and/or L is —NR₄C(O)—.

In certain embodiments, Ring G is substituted, such as with one or more (e.g., two) alkoxy (e.g., methoxy, ethoxy) groups, for example, in the positions ortho to the bridgehead carbon atoms. In particular embodiments, Ring G is substituted when R₂ is an unsubstituted alkyl group, L′ is —C(O)O—, R₃ is a substituted or unsubstituted aryl group and/or L is —NR₄C(O)—.

TABLE 3

| COMPOUND NO | STRUCTURE | ED₅₀ |
|---|---|---|
| 1 | 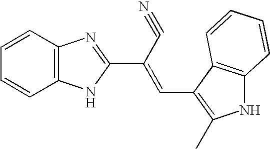 | C |
| 2 | 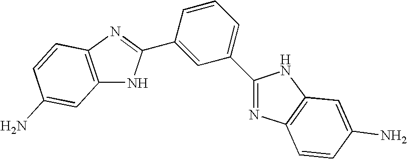 | B |
| 3 | 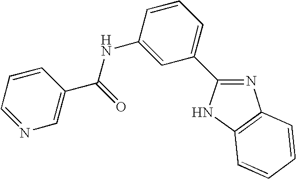 | D |
| 4 | 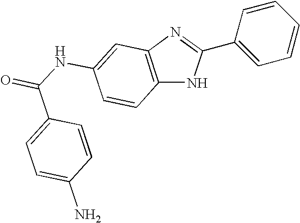 | B |
| 5 | 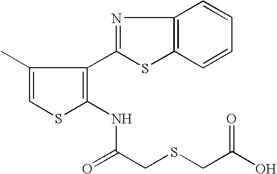 | A |
| 6 | 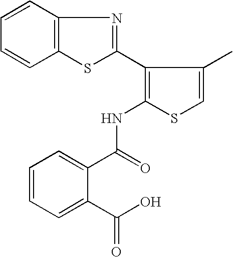 | A |
| 7 | 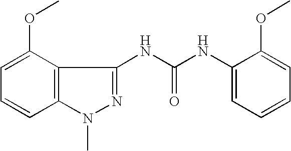 | C |
| 8 | 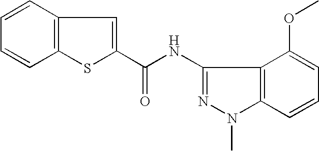 | D |
| 9 | 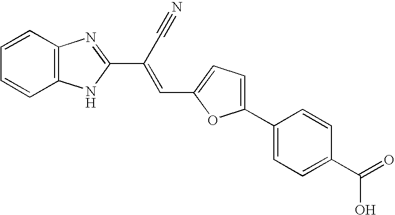 | A |
| 10 | 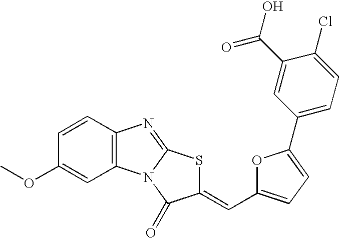 | A |
| 11 | 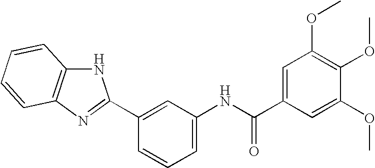 | B |
| 12 | 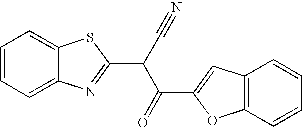 | B |
| 13 | 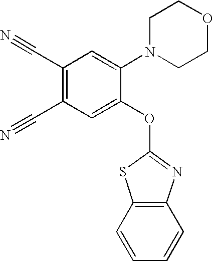 | C |
| 14 | 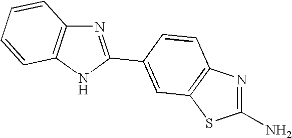 | C |
| 15 | 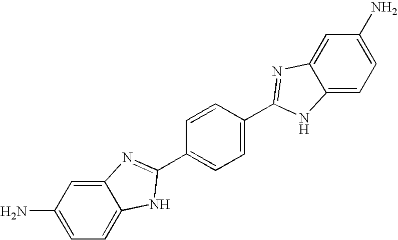 | B |
| 16 | 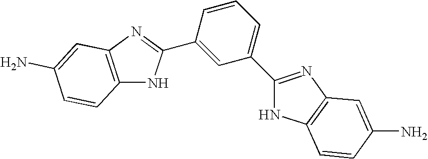 | B |
| 17 | 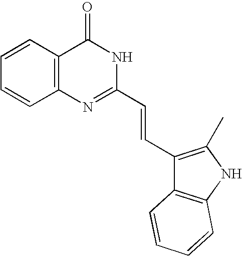 | D |
| 18 | 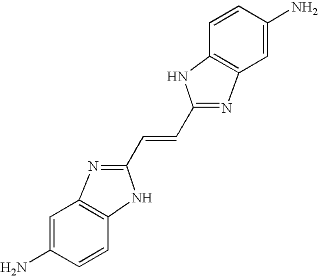 | B |
| 19 | 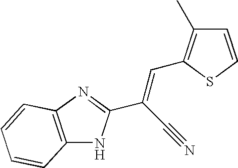 | C |
| 20 | 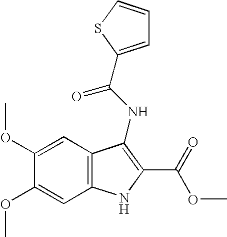 | C |
| 21 | 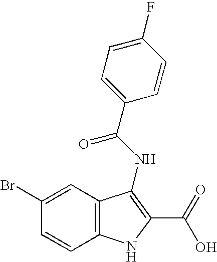 | B |
| 22 | 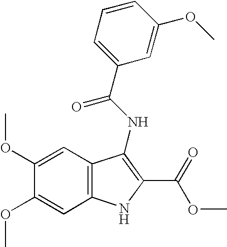 | D |
| 23 | 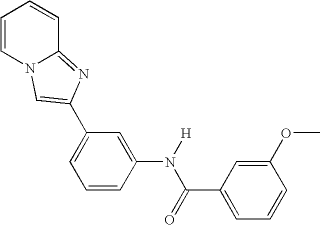 | D |
| 24 | 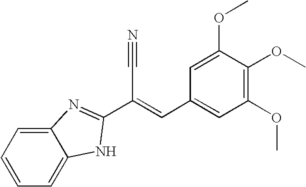 | C |
| 25 | 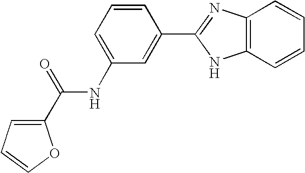 | B |
| 26 | 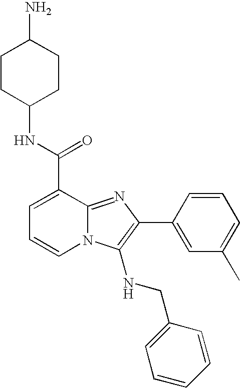 | C |
| 27 | 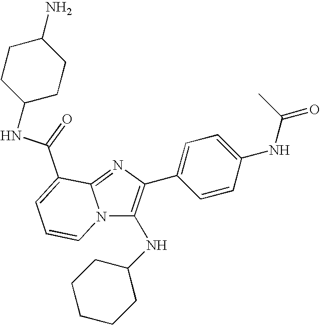 | B |
| 28 | 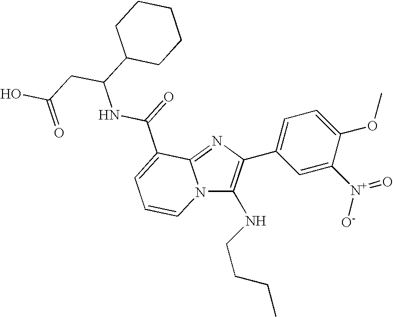 | C |
| 29 | 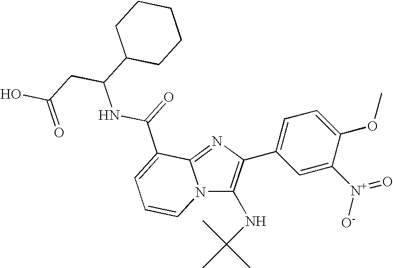 | D |
| 30 | 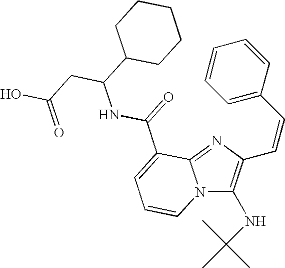 | B |
| 31 | 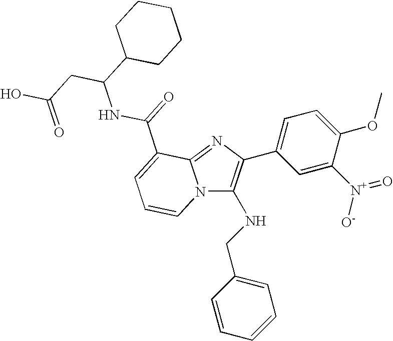 | D |
| 32 | 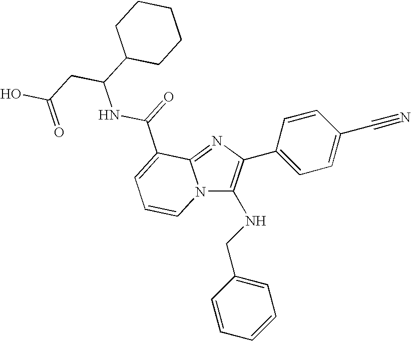 | B |
| 33 | 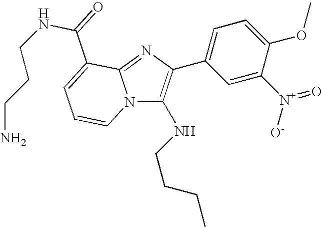 | C |
| 34 | 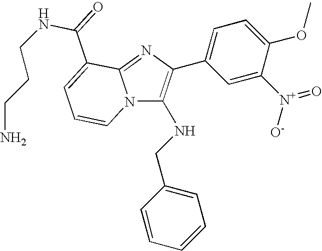 | C |
| 35 | 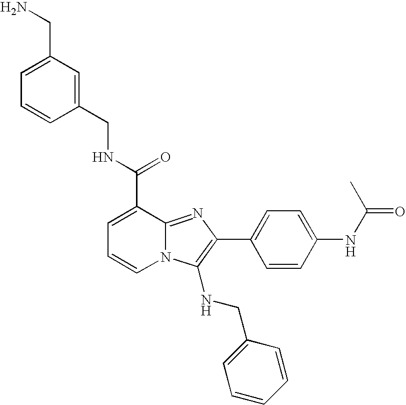 | B |
| 36 | 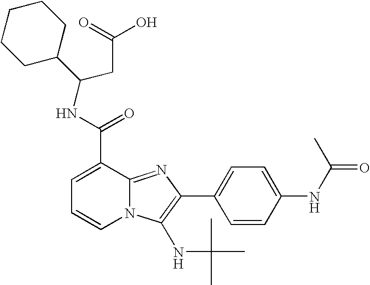 | C |
| 37 | 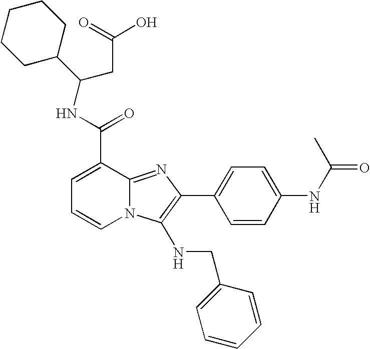 | B |
| 38 | 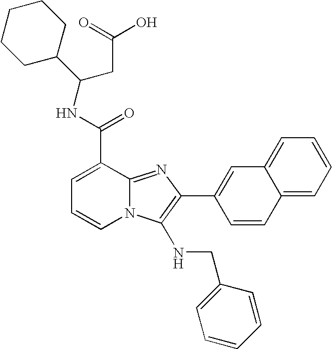 | D |
| 39 | 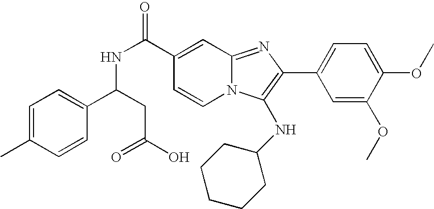 | C |
| 40 | 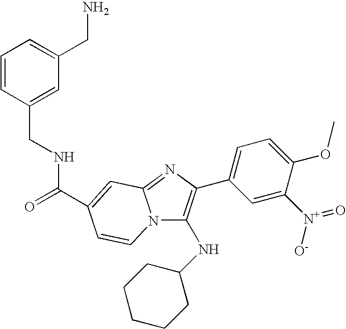 | C |
| 41 | 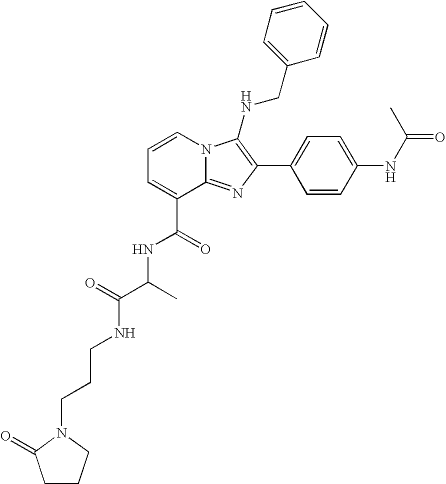 | C |
| 42 | 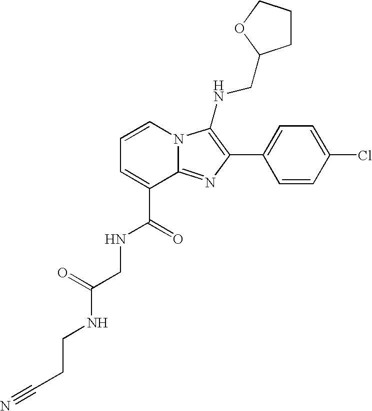 | B |
| 43 | 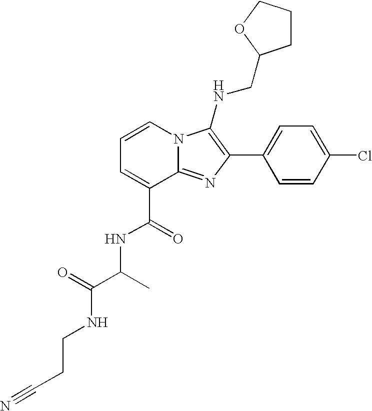 | A |
| 44 | 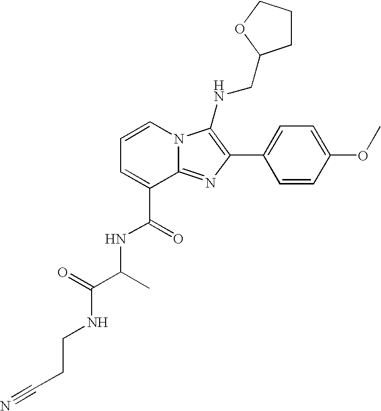 | C |
| 45 | 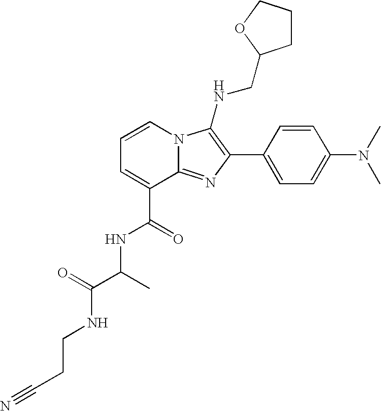 | B |
| 46 | 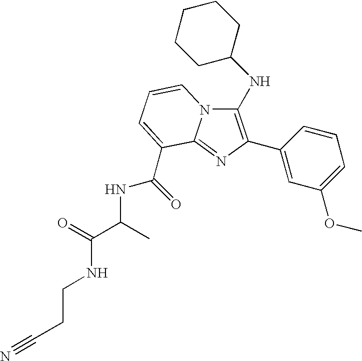 | B |
| 47 | 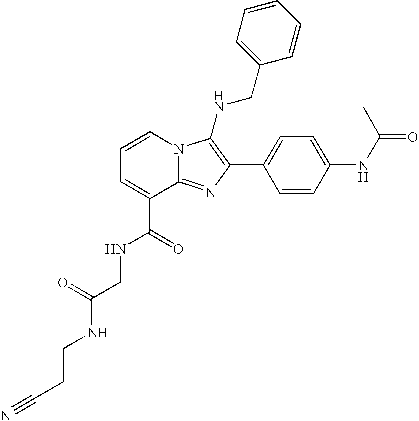 | B |
| 48 | 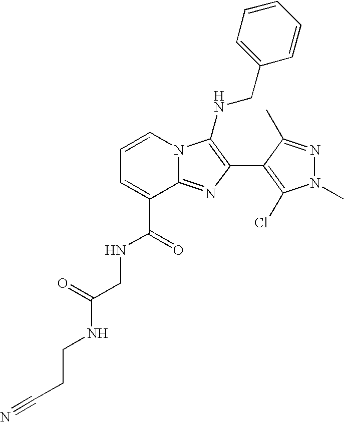 | C |
| 49 | 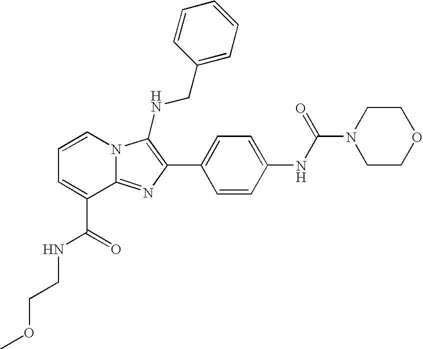 | B |
| 50 | 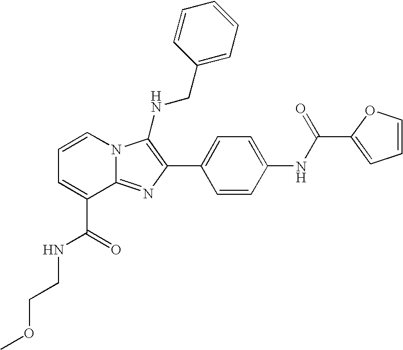 | D |
| 51 | 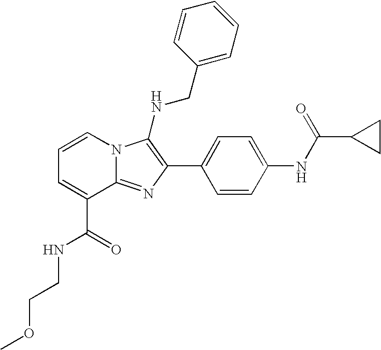 | B |
| 52 | 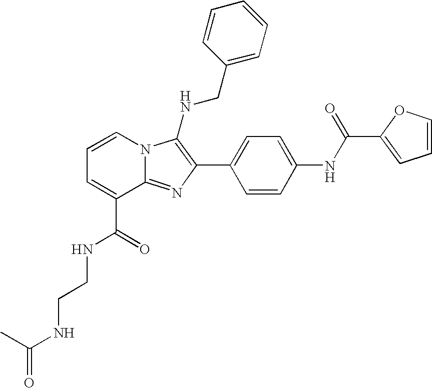 | C |
| 53 | 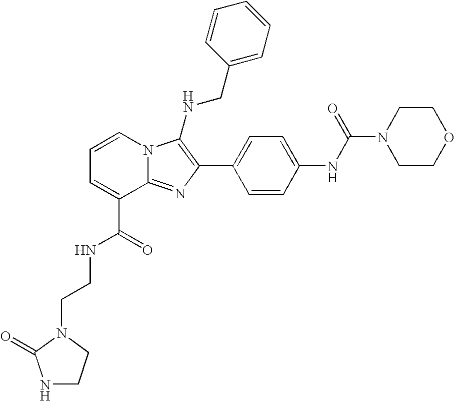 | C |
| 54 | 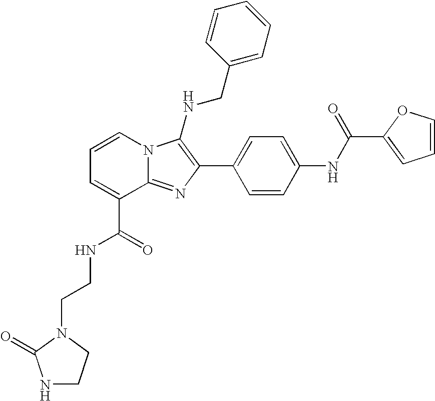 | D |
| 55 | 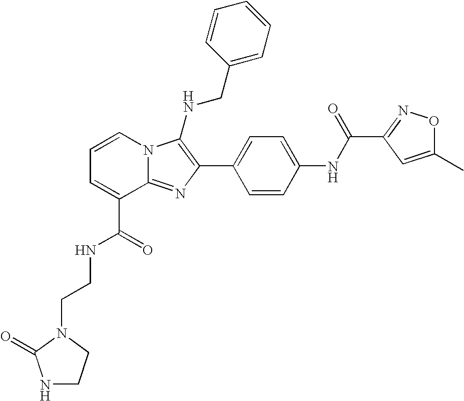 | D |
| 56 | 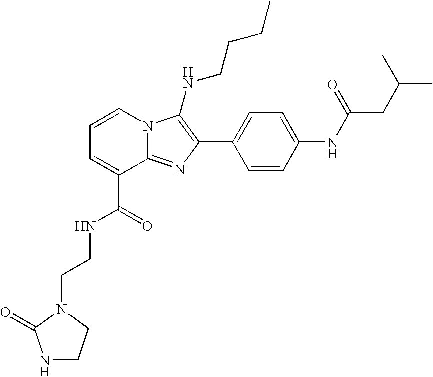 | D |
| 57 | 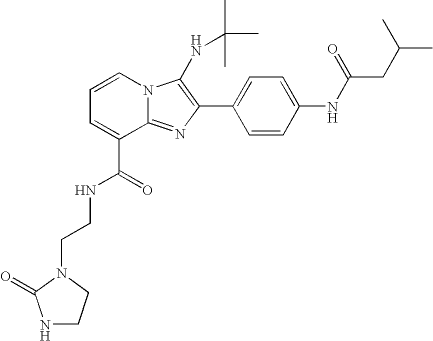 | D |
| 58 | 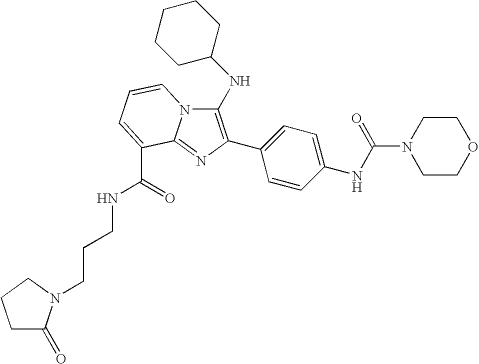 | A |
| 59 | 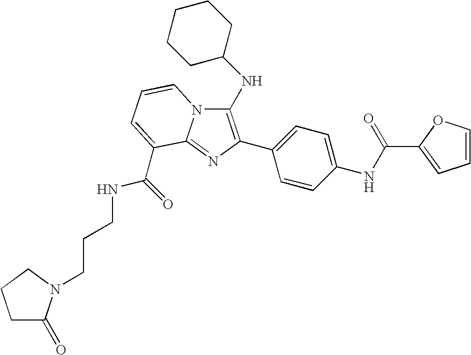 | C |
| 60 | 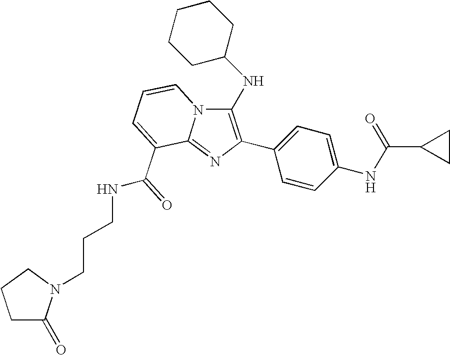 | B |
| 61 | 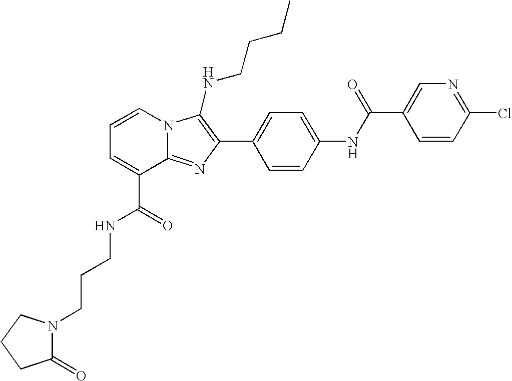 | D |
| 62 | 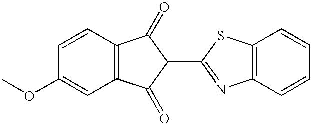 | A |
| 63 | 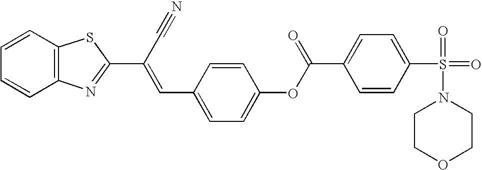 | N/A |
| 64 | 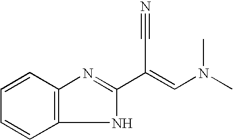 | B |
| 65 | 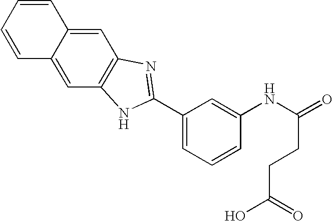 | D |
| 66 | 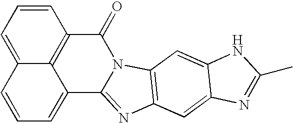 | C |
| 91 | 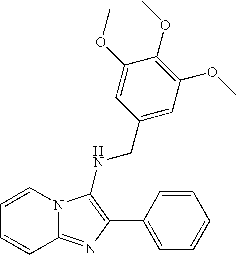 | |
| 92 | 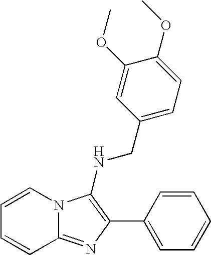 | |
| 93 | 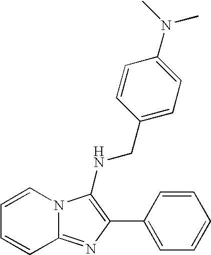 | |
| 94 | 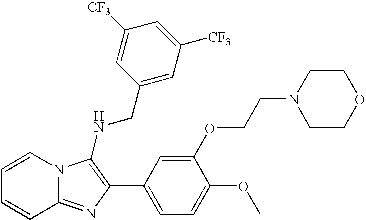 | |
| 95 | 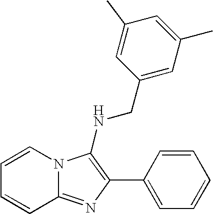 | |
| 96 | 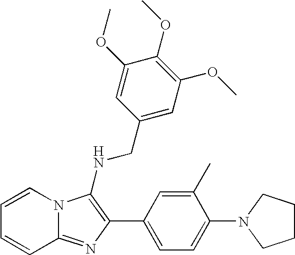 | |
| 97 | 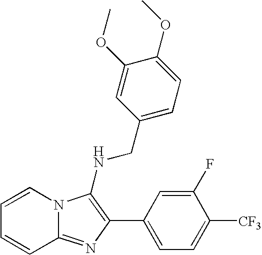 | |
| 98 | 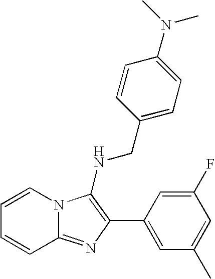 | |
| 99 | 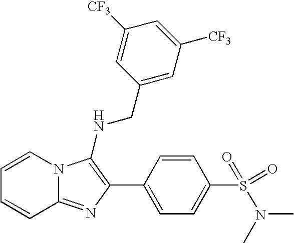 | |
| 100 | 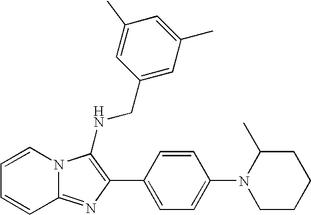 | |
| 101 | 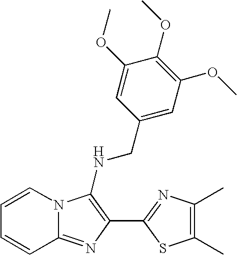 | |
| 102 | 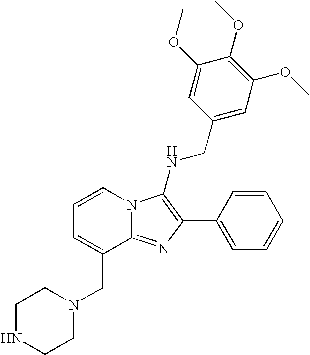 | |
| 103 | 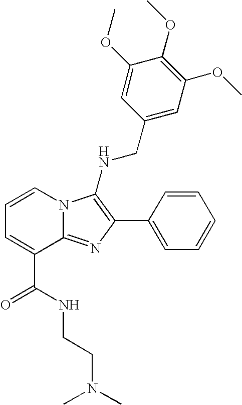 | |
| 104 | 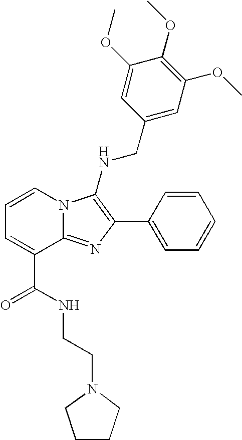 | |
| 105 | 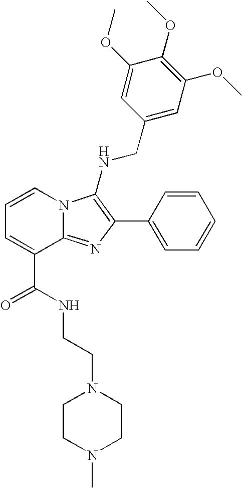 | |
| 106 | 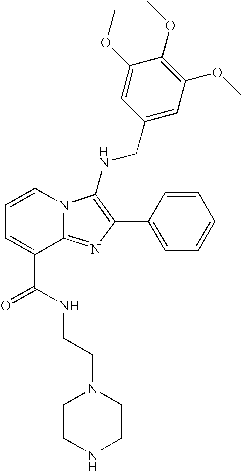 | |
| 107 | 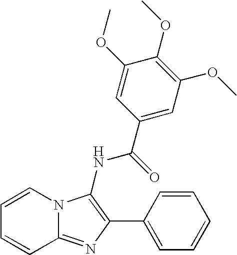 | |
| 108 | 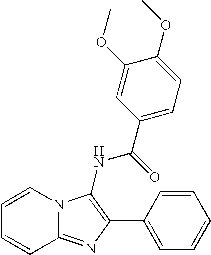 | |
| 109 | 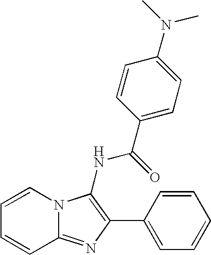 | |
| 110 | 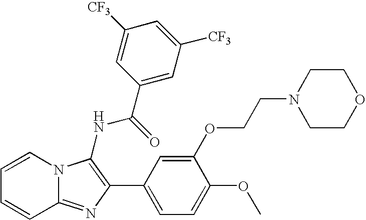 | |

TABLE 4

| COMPOUND | | |
|---|---|---|
| NO | STRUCTURE | IC₅₀ |
| 84 | 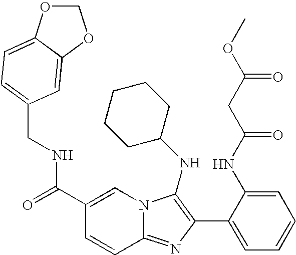 | A |

TABLE 5

| COMPOUND NO | STRUCTURE | ED₅₀ |
|---|---|---|
| 5 | 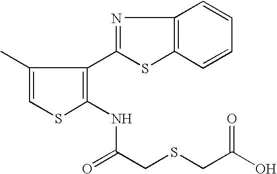 | C |
| 6 | 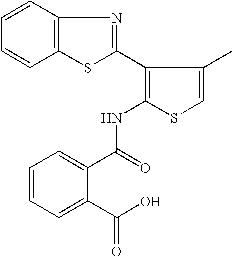 | N/A |
| 14 | 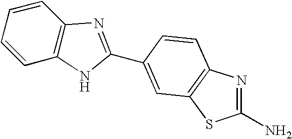 | C |
| 29 | 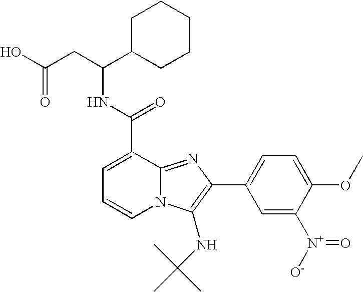 | D |
| 30 | 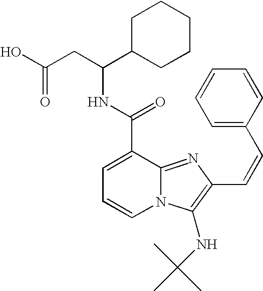 | N/A |
| 31 | 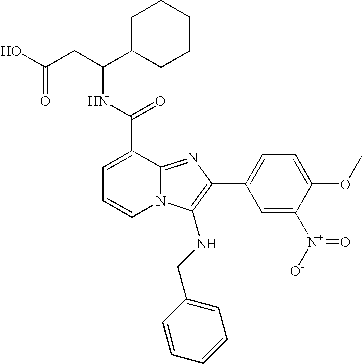 | N/A |
| 32 | 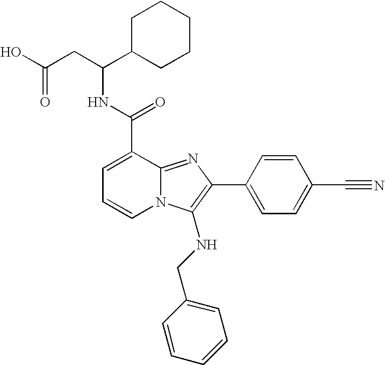 | A |
| 37 | 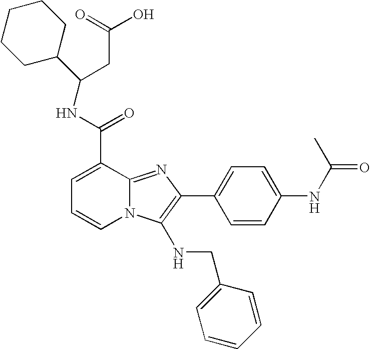 | C |
| 43 | 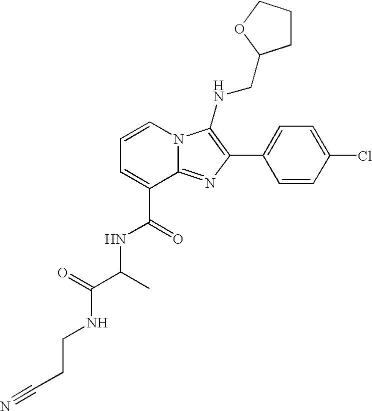 | B |
| 45 | 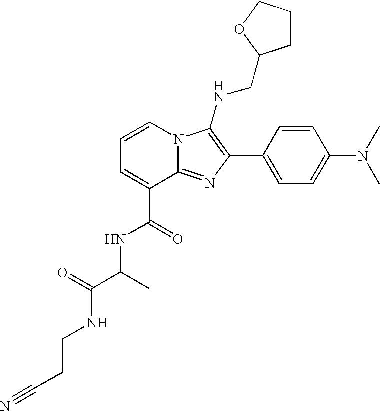 | D |
| 67 | 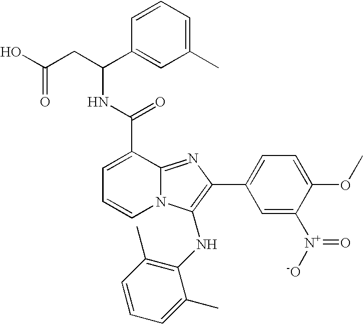 | D |
| 68 | 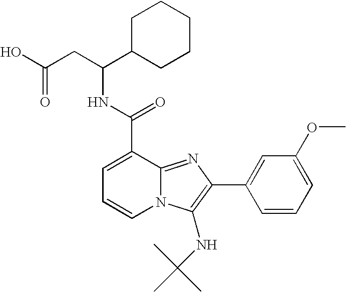 | N/A |
| 69 | 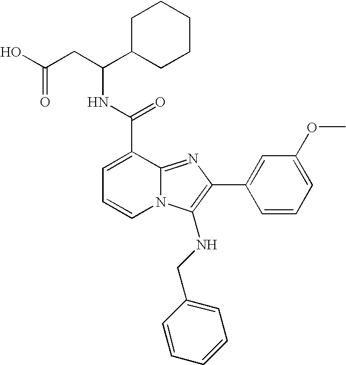 | B |
| 70 | 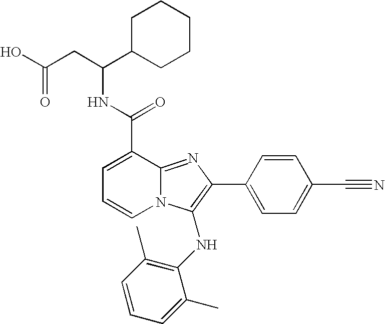 | D |
| 71 | 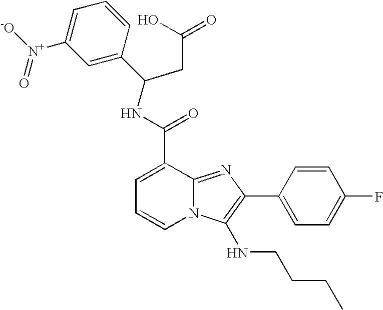 | B |
| 72 | 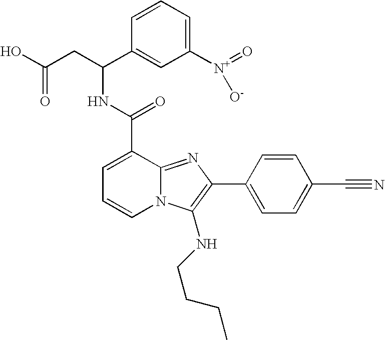 | B |
| 73 | 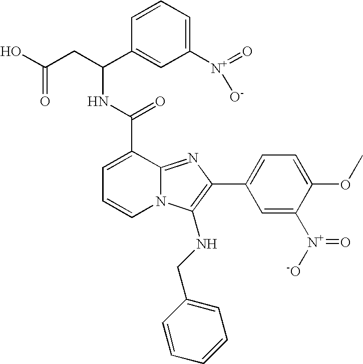 | B |
| 74 | 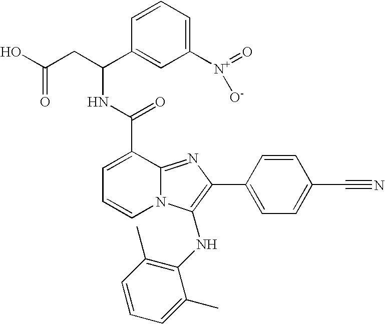 | D |
| 75 | 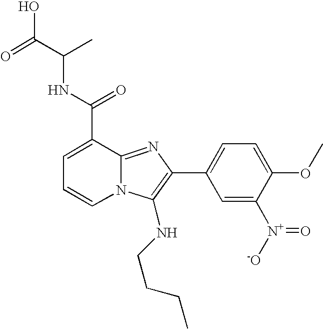 | B |
| 76 | 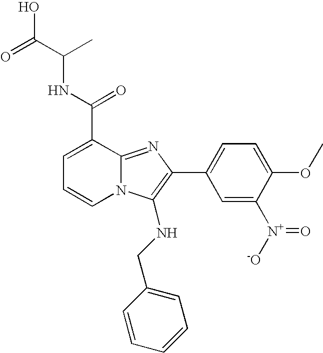 | A |
| 77 | 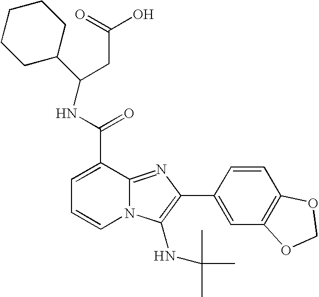 | N/A |
| 78 | 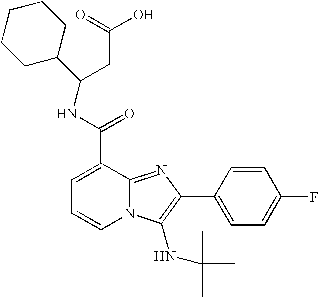 | C |
| 79 | 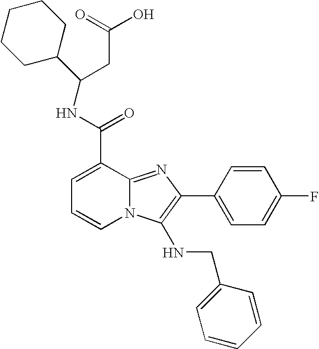 | B |
| 80 | 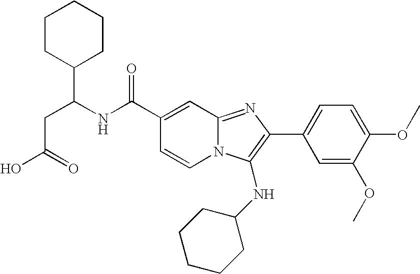 | B |
| 81 | 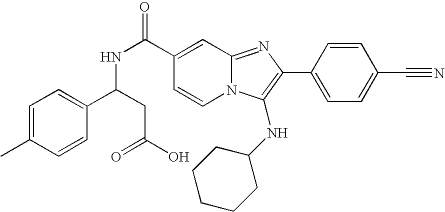 | B |
| 82 | 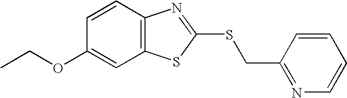 | N/A |
| 83 | 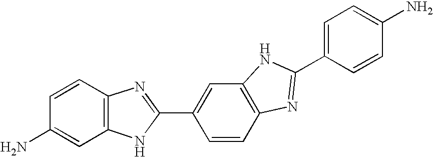 | D |

TABLE 6

| COMPOUND NO | STRUCTURE | IC₅₀ |
|---|---|---|
| 85 | 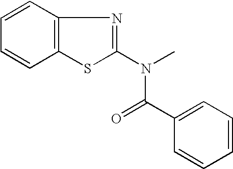 | N/A |
| 86 | 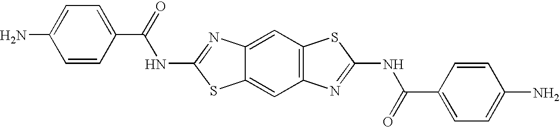 | B |
| 87 | 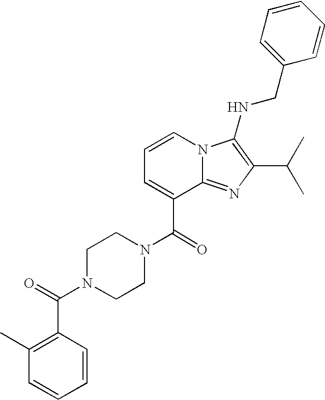 | C |
| 88 | 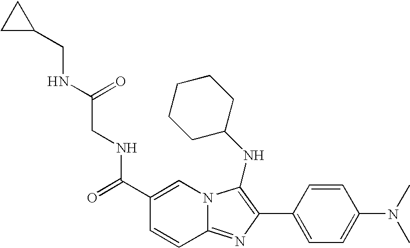 | A |
| 89 | 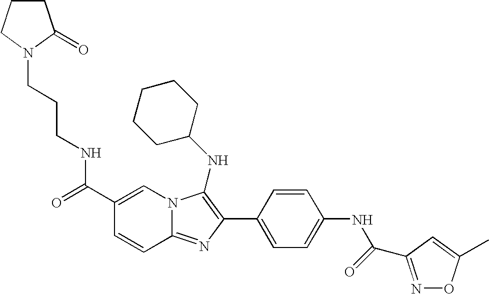 | C |
| 90 | 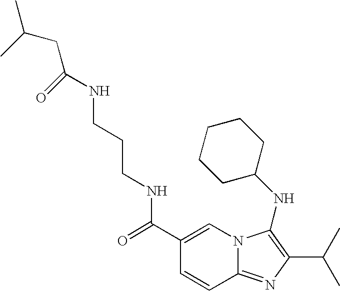 | N/A |

### 2. Modulators

In one embodiment, [symbol] -modulating compounds of the invention are represented by Structural Formula (I):

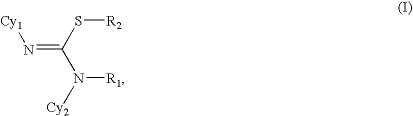

or a salt thereof, where:

Cy₁ and Cy₂ are independently a substituted or unsubstituted cyclic group; and

R₁ and R₂ are independently —H, a substituted or unsubstituted alkyl group, a substituted or unsubstituted aryl group or a substituted or unsubstituted non-aromatic heterocyclic group, or R₁ and R₂ taken together with the atoms to which they are attached from an optionally substituted heterocycle.

In certain embodiments, Cy₁ and Cy₂ are independently a substituted or unsubstituted 5- or 6-membered cyclic group, particularly a substituted or unsubstituted 6-membered cyclic group, such as a substituted or unsubstituted phenyl, pyridyl or cyclohexyl group.

In certain embodiments, R₁ and R₂ taken together with the atoms to which they are attached from an optionally substituted heterocycle, typically a 5- or 6-membered heterocycle. Such heterocycles can be saturated or unsaturated (e.g., aromatic), but are typically saturated (i.e., have no multiple bonds). In particular embodiments, R₁ and R₂ taken together with the atoms to which they are attached from an optionally substituted heterocycle and Cy₁ and Cy₂ are independently a substituted or unsubstituted 5- or 6-membered cyclic group.

In another embodiment, [symbol] -modulating compounds of the invention are represented by Structural Formula (II):

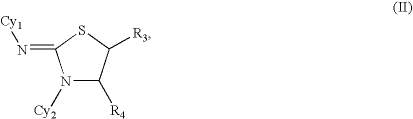

or a salt thereof, where:

Cy₁ and Cy₂ are independently a substituted or unsubstituted cyclic group;

R₃ and R₄ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NRC(O)R′, —NO₂, ═O and ═S; and

R and R′ are independently —H, a substituted or unsubstituted alkyl group, a substituted or unsubstituted aryl group or a substituted or unsubstituted non-aromatic heterocyclic group.

In certain embodiments, R₃ is a substituted alkyl group, such as a carboxyalkyl group (e.g., carboxymethyl, carboxyethyl).

In certain embodiments, R₄ is ═O. In particular embodiments, R₄ is ═O and R₃ is a substituted alkyl group.

In certain embodiments, Cy₁ and Cy₂ are independently a substituted or unsubstituted 5- or 6-membered cyclic group, particularly a substituted or unsubstituted 6-membered cyclic group, such as a substituted or unsubstituted phenyl, pyridyl or cyclohexyl group. In particular embodiments, Cy₁ and Cy₂ are independently a substituted or unsubstituted 5- or 6-membered cyclic group when R₄ is ═O and/or R₃ is a substituted alkyl group. Preferred compounds are selected such that Cy₁ and Cy₂ are independently a substituted or unsubstituted 5- or 6-membered cyclic group, R₄ is ═O and R₃ is a substituted alkyl group.

In certain embodiments, the compounds of the invention exclude those species disclosed in the Table 2.
[symbol] -modulating compounds of the invention having hydroxyl substituents, unless otherwise indicated, also include the related secondary metabolites, particularly sulfate, phosphate, acyl (e.g., acetyl, fatty acid acyl) and sugar (e.g., glucurondate, glucose) derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

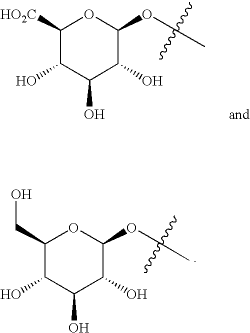

These groups are generally cleavable to —OH by hydrolysis or by metabolic (e.g., enzymatic) cleavage.
[symbol] compounds of the invention advantageously modulate the level and/or activity of a protein, particularly the deacetylase activity of the protein.

Separately or in addition to the above properties, certain [symbol] modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

[symbol]

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A 5- to 7-membered ring includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, ═O, ═S, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(—NH)—NH₂, —NH—C(═NH)—NHR^{a}, NH—C(═NH)—N(R^{a}R^{b}), NH—C(═NR^{c})—NH₂, —NH—C(═NR^{c})—NHR^{a}, —NH—C(═NR^{c})—N(R^{a}R^{b}), —N^{d}H—C(═NH)—NH₂, —NR^{d}—C(═NH)—NHR^{a}, —NR^{d}C(═NH)—N(R^{a}R^{b}), —NR^{d}—C(═NR^{c})—NH₂, —NR^{d}—C(═NR^{c})—NHR^{a}, —N^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂.

TABLE 2

| COMPOUND NO | STRUCTURE | ED₅₀ |
|---|---|---|
| 1 | 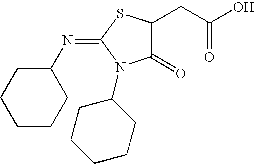 | A |
| 2 | 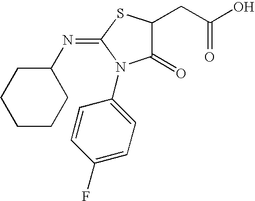 | A |

### 2. Modulators

In one embodiment, [symbol] modulating compounds of the invention are represented by Structural Formula (Ia):

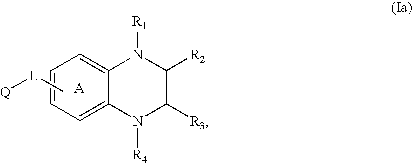

or a salt thereof, wherein:

Ring A optionally has one or more substituents in addition to -L-Q;

L is —C(O)NR—, —NRC(O)—, —NR—NR′—C(O)—, —C(O)—NR—NR′— or —CHR═CHR′—;

Q is a substituted or unsubstituted alkyl group or a substituted or unsubstituted cyclic group, or Q and R taken together with the atoms to which they are attached form a heterocyclic group;

R₁ is —H, a substituted or unsubstituted acyl group, a substituted or unsubstituted alkyl group, a substituted or unsubstituted aryl group or a substituted or unsubstituted non-aromatic heterocyclic group, or R₁ and R₂ taken together with the atoms to which they are attached form an optionally substituted fused ring;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NRC(O)R′, —NO₂, ═O and ═S;

R₄ is —H, a substituted or unsubstituted alkyl group, a substituted or unsubstituted alkenyl group, a substituted or unsubstituted aryl group or a substituted or unsubstituted non-aromatic heterocyclic group;

R and R′ are independently —H, a substituted or unsubstituted alkyl group, a substituted or unsubstituted aryl group or a substituted or unsubstituted non-aromatic heterocyclic group; and

n is 1 or 2.

In certain embodiments, R₁ and R₂ taken together with the atoms to which they are attached form an optionally substituted fused ring, typically a 5- or 6-membered ring. One group of compounds having these values of R₁ and R₂ is represented by Structural Formula (IV):

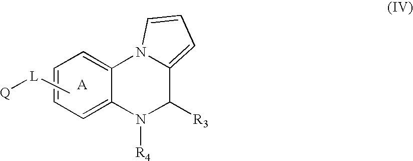

Compounds represented by Structural Formula (IV) generally have L as an amide group, either where the carbonyl group (—C(O)NR—) or the nitrogen atom (—NRC(O)—) attaches to Ring A. One group of compounds having such an amide linkage is represented by Structural Formula (V):

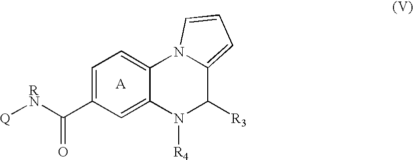

In certain embodiments, Q and R taken together with the atoms to which they are attached form a heterocyclic group, typically a non-aromatic heterocyclic group. Such groups advantageously include two nitrogen atoms. An exemplary group of compounds having such a heterocyclic group are represented by Structural Formula (VI):

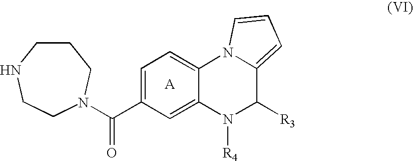

For compounds represented by Structural Formula (VI), R₄ is typically a substituted alkyl group, such as an aralkyl group. Separately or in addition, R₃ is typically ═O. For compounds with these values of R₃ and/or R₄, R₂ is typically —H or a substituted or unsubstituted alkyl group (e.g., methyl, ethyl, n-propyl, isopropyl).

In other embodiments involving compounds represented by Structural Formula (V), Q is a substituted or unsubstituted alkyl group, particularly an aminoalkyl group. R₂, R₃ and R₄ generally have the values indicated in the prior paragraph.

In certain embodiments, compounds represented by Structural Formula (Ia) are selected such that Q is a substituted or unsubstituted alkyl group; R₁ is —H, a substituted or unsubstituted acyl group, a substituted or unsubstituted alkyl group, a substituted or unsubstituted aryl group or a substituted or unsubstituted non-aromatic heterocyclic group; R₄ is —H, a substituted or unsubstituted alkyl group, a substituted or unsubstituted aryl group or a substituted or unsubstituted non-aromatic heterocyclic group.

Typically, L in such compounds is —NRC(O)—.

In certain embodiments, R₁ is a substituted acyl group, such as an amino-substituted acyl group (e.g., an aralkylamino-substituted group or an aryloxyalkylamino-substituted acyl group). In particular embodiments, R₁ is a substituted acyl group when L is —NRC(O)— and/or Q is a substituted or unsubstituted alkyl group.

In certain embodiments, R₄ is a substituted alkyl group, such as an aralkyl group (e.g., pyridylalkyl, for example, pyridylethyl). In particular embodiments, R₄ is a substituted alkyl group when R₁ is a substituted acyl group, L is —NRC(O)— and/or Q is a substituted or unsubstituted alkyl group.

In certain embodiments, R₃ is ═O. In particular embodiments, R₃ is ═O when R₄ is a substituted alkyl group, R₁ is a substituted acyl group, L is —NRC(O)— and/or Q is a substituted or unsubstituted alkyl group.

In certain embodiments, R₂ is —H. In particular embodiments, R₂ is —H when R₃ is ═O, R₄ is a substituted alkyl group, R₁ is a substituted acyl group, L is —NRC(O)— and/or Q is a substituted or unsubstituted alkyl group. Preferred compounds are selected such that R₂ is —H, R₃ is ═O, R₄ is a substituted alkyl group, R₁ is a substituted acyl group, L is —NRC(O)— and/or Q is a substituted or unsubstituted alkyl group.

In certain embodiments, [symbol] modulating compounds of the invention are represented by Structural Formula (I):

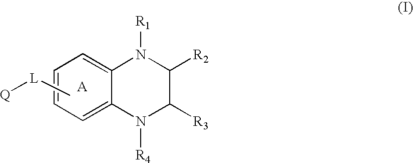

or a salt thereof, wherein:

Ring A optionally has one or more substituents in addition to -L-Cy₁;

L is —C(O)NR—, —NRC(O)—, —NR—NR′—C(O)—, —C(O)—NR—NR′— or —CHR═CHR′—;

Q is a substituted or unsubstituted alkyl group (e.g., an aminoalkyl group) or a substituted or unsubstituted cyclic group;

R₁ is —H, a substituted or unsubstituted acyl group, a substituted or unsubstituted alkyl group, a substituted or unsubstituted aryl group or a substituted or unsubstituted non-aromatic heterocyclic group, or R₁ and R₂ taken together with the atoms to which they are attached form an optionally substituted fused ring;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)NRR′, —NRR′, —NRC(O)OR′, —NRC(O)R′, —NO₂, ═O and ═S;

R₄ is —H, a substituted or unsubstituted alkyl group, a substituted or unsubstituted aryl group or a substituted or unsubstituted non-aromatic heterocyclic group;

R and R′ are independently —H, a substituted or unsubstituted alkyl group, a substituted or unsubstituted aryl group or a substituted or unsubstituted non-aromatic heterocyclic group; and

n is 1 or 2.

In another embodiment, the invention provides [symbol] modulating compounds of Structural Formula (II):

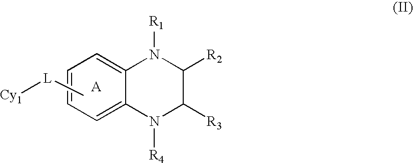

or a salt thereof, wherein:

Ring A optionally has one or more substituents in addition to -L-Cy₁;

Cy₁ is a substituted or unsubstituted cyclic group;

L is —C(O)NR—, —NRC(O)—, —NR—NR′—C(O)—, —C(O)—NR—NR′— or —CHR═CHR′—;

R₁ is —H, a substituted or unsubstituted acyl group, a substituted or unsubstituted alkyl group, a substituted or unsubstituted aryl group or a substituted or unsubstituted non-aromatic heterocyclic group;

R₂ and R₃ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NRC(O)R′, —NO₂, ═O and ═S;

R₄ is —H, a substituted or unsubstituted alkyl group, a substituted or unsubstituted aryl group or a substituted or unsubstituted non-aromatic heterocyclic group;

R and R′ are independently —H, a substituted or unsubstituted alkyl group, a substituted or unsubstituted aryl group or a substituted or unsubstituted non-aromatic heterocyclic group; and

n is 1 or 2.

In certain embodiments, Cy₁ is a substituted or unsubstituted aryl group such as a substituted or unsubstituted pyridyl, phenyl or isoxazolyl group.

In certain embodiments, L is —NRC(O)—. In particular embodiments, L is —NRC(O)— and Cy₁ is a substituted or unsubstituted aryl group.

In certain embodiments, R₁ is a substituted acyl group, such as an amino-substituted acyl group (e.g., an aralkylamino-substituted group or an aryloxyalkylamino-substituted acyl group). In particular embodiments, R₁ is a substituted acyl group when L is —NRC(O)— and/or Cy₁ is a substituted or unsubstituted aryl group.

In certain embodiments, R₄ is a substituted alkyl group, such as an aralkyl group (e.g., pyridylalkyl, for example, pyridylethyl). In particular embodiments, R₄ is a substituted alkyl group when R₁ is a substituted acyl group, L is —NRC(O)— and/or Cy₁ is a substituted or unsubstituted aryl group.

In certain embodiments, R₃ is ═O. In particular embodiments, R₃ is ═O when R₄ is a substituted alkyl group, R₁ is a substituted acyl group, L is —NRC(O)— and/or Cy₁ is a substituted or unsubstituted aryl group.

In certain embodiments, R₂ is —H. In particular embodiments, R₂ is —H when R₃ is ═O, R₄ is a substituted alkyl group, R₁ is a substituted acyl group, L is —NRC(O)— and/or Cy₁ is a substituted or unsubstituted aryl group. Preferred compounds are selected such that R₂ is —H, R₃ is ═O, R₄ is a substituted alkyl group, R₁ is a substituted acyl group, L is —NRC(O)— and/or Cy₁ is a substituted or unsubstituted aryl group.

In yet another embodiment, [symbol] -modulating compounds of the invention are represented by Structural Formula (III):

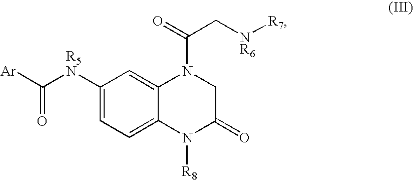

or a salt thereof wherein:

Ar is a substituted or unsubstituted aryl group;

R₅, R₆, R₇ and R₈ are independently —H, a substituted or unsubstituted alkyl group, a substituted or unsubstituted aryl group or a substituted or unsubstituted non-aromatic heterocyclic group.

In certain embodiments, R₇ is a substituted alkyl group, such as an aralkyl or aryloxyalkyl group (e.g., pyridylalkyl, for example, pyridylethyl; phenylalkyl, for example, phenylethyl; phenoxyalkyl, for example, phenoxyethyl).

In certain embodiments, R₈ is a substituted alkyl group, such as an aralkyl group (e.g., pyridylalkyl, for example, pyridylethyl). In a particular embodiment, R₈ is a substituted alkyl group and R₇ is a substituted alkyl group.

In certain embodiments, R₅ and/or R₆ are —H. Preferably, both R₅ and R₆ are —H. In particular embodiments, R₅ and/or R₆ are —H when R₈ is a substituted alkyl group and/or R₇ is a substituted alkyl group.

In certain embodiments, Ar is a substituted or unsubstituted phenyl, pyridyl or isoxazolyl group.

In certain embodiments, the compounds of the invention exclude those species disclosed in Tables 3 and 4.

Novel compounds of the invention can also be used in the methods described above.
[symbol] modulating compounds of the invention having hydroxyl substituents, unless otherwise indicated, also include the related secondary metabolites, particularly sulfate, acyl (e.g., acetyl, fatty acid acyl) and sugar (e.g., glucurondate, glucose) derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

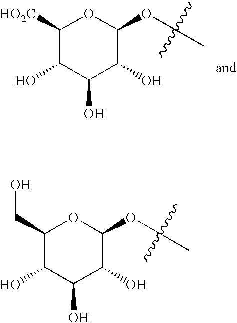

These groups are generally cleavable to —OH by hydrolysis or by metabolic (e.g., enzymatic) cleavage.
[symbol] modulating compounds of the invention advantageously [symbol] modulate the level and/or activity of a protein.

Separately or in addition to the above properties, certain [symbol] modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity, [symbol] ). An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

An acyl group has a carbonyl group attached to a carbon atom in an aliphatic or aromatic group and is attached to another moiety by the carbonyl group.

A cyclic group includes carbocyclic and heterocyclic groups. Such groups can be saturated or unsaturated, including aromatic. Heterocyclic groups typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, ═O, ═S, halogen (—Br, —Cl,

| COMPOUND NO | STRUCTURE | ED₅₀ |
|---|---|---|
| 1 | 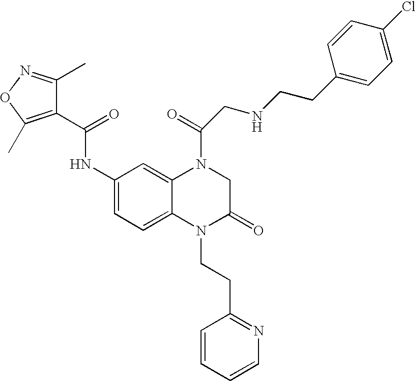 | B |
| 2 | 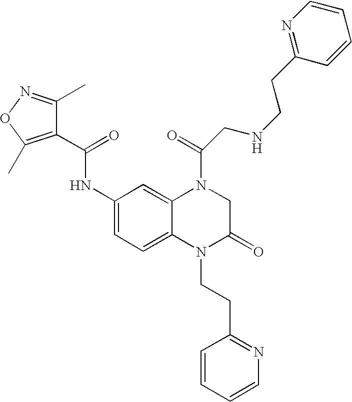 | B |
| 3 | 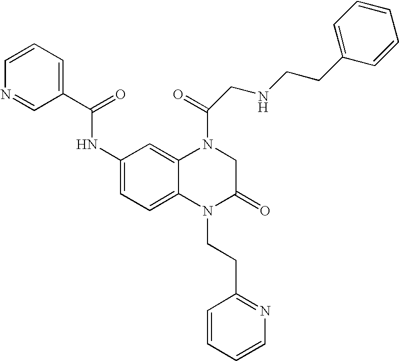 | B |
| 4 | 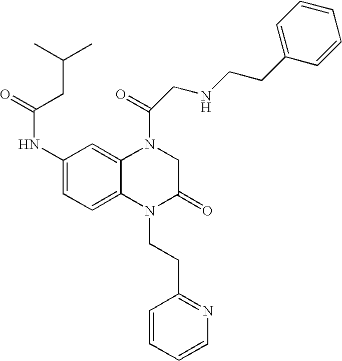 | B |
| 5 | 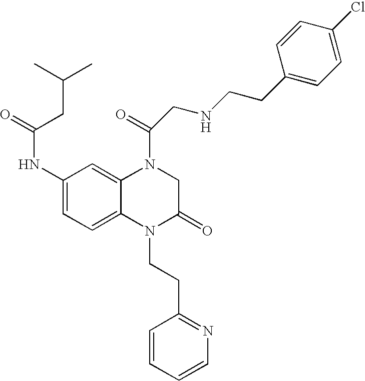 | C |
| 6 | 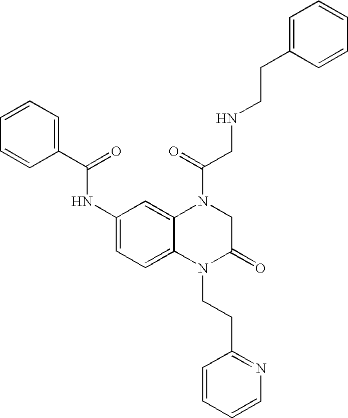 | B |
| 7 | 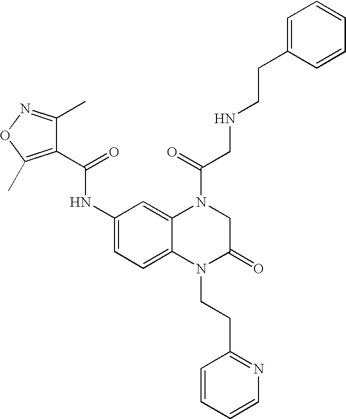 | B |
| 8 | 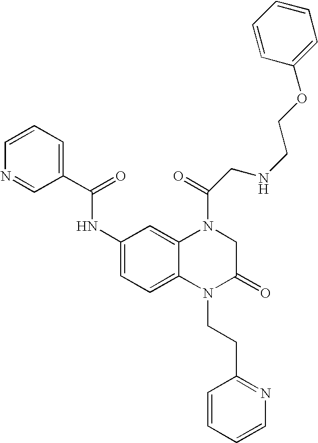 | B |
| 9 | 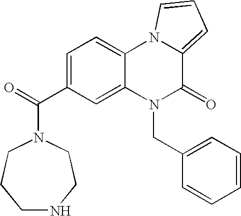 | A |
| 10 | 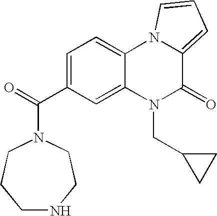 | D |
| 11 | 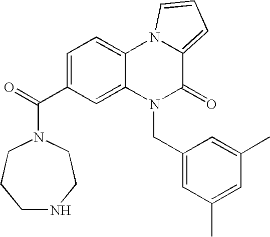 | D |
| 12 | 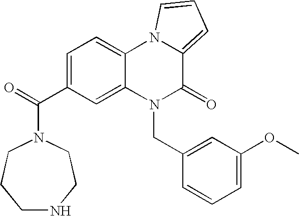 | A |
| 13 | 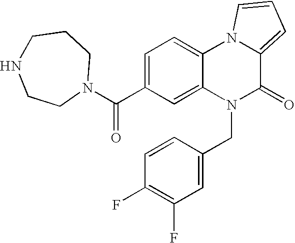 | A |
| 14 | 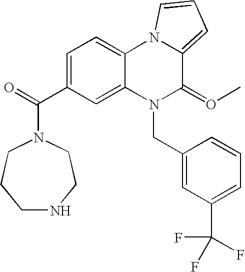 | A |
| 15 | 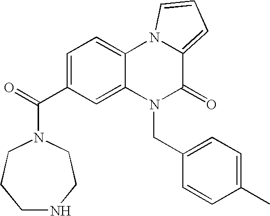 | A |
| 16 | 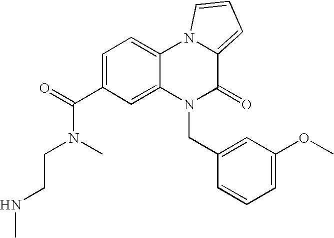 | A |
| 17 | 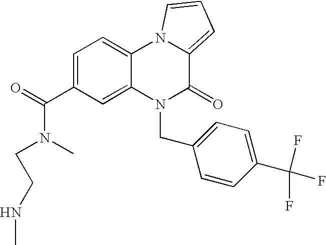 | A |
| 18 | 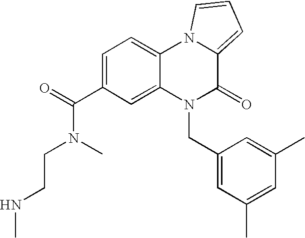 | A |
| 19 | 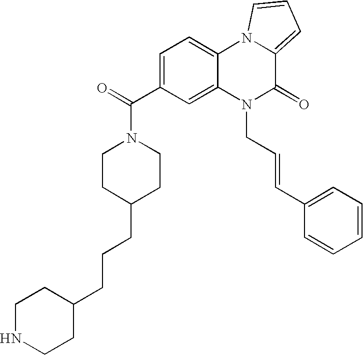 | B |
| 20 | 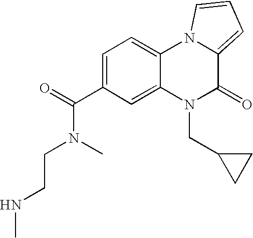 | A |
| 21 | 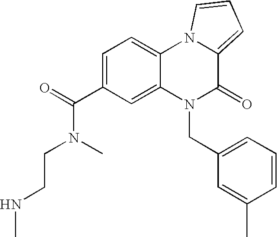 | B |
| 22 | 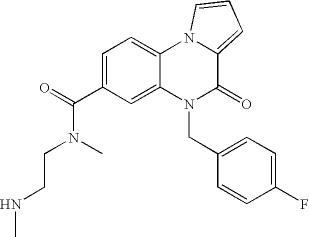 | A |

TABLE 4

| COMPOUND NO | STRUCTURE | ED₅₀ |
|---|---|---|
| 9 | 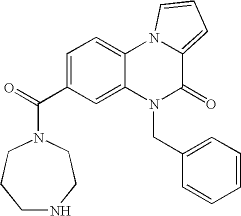 | B |
| 10 | 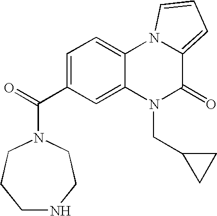 | D |
| 11 | 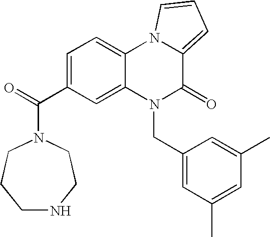 | A |
| 12 | 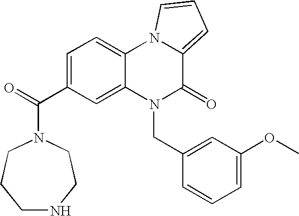 | A |
| 13 | 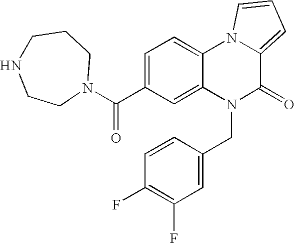 | D |
| 14 | 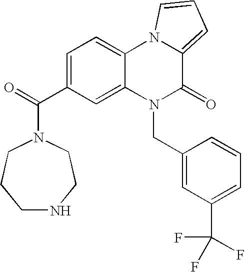 | A |
| 15 | 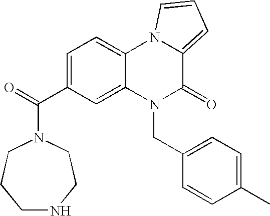 | D |
| 17 | 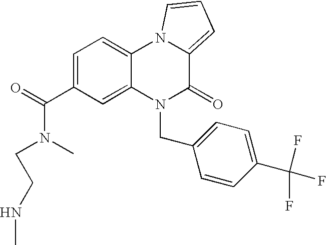 | D |
| 23 | 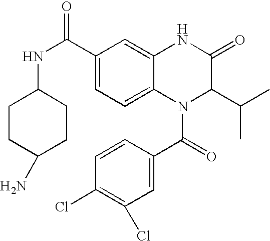 | B |

### 2. Modulators

In one embodiment, [symbol] -modulating compounds of the invention are represented by Structural Formula (Ia):

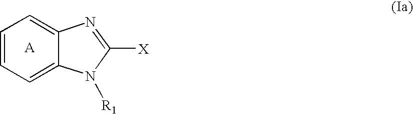

or a salt thereof, wherein:

Ring A is optionally substituted;

R₁ is —H, a substituted or unsubstituted alkyl group, a substituted or unsubstituted alkenyl group, a substituted or unsubstituted aryl group or a substituted or unsubstituted heterocyclic group;

X is —(CHR₂)ₘ—NHCO—R₃, —(CHR₂)ᵣ—NHCONR₄R₅, —(CH₂)ᵣ—NR₁SO₂—R₃, —SR₃ or -arylene-R₂;

each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₃ is a substituted or unsubstituted alkyl group, a substituted or unsubstituted aryl group or a substituted or unsubstituted heterocyclic group;

R₄ and R₅ are independently —H, a substituted or unsubstituted alkyl group, a substituted or unsubstituted aryl group or a substituted or unsubstituted non-aromatic heterocyclic group;

m is an integer from 2 to 12;

n is 1 or 2; and

r is an integer from 0 to 12.

Typically, Ring A is directly or indirectly substituted with a carboxy group. In an exemplary embodiment, such compounds are represented by Structural Formula (IV):

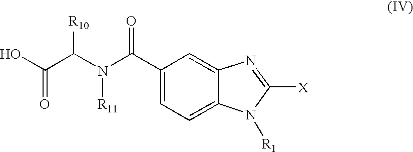

where R₁₀ is selected from the group consisting of —H, a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂, or R₁₀ and R₁₁ taken together with the atoms to which they are attached form a non-aromatic heterocyclic ring; and R₁₁ is —H, a substituted or unsubstituted alkyl group, a substituted or unsubstituted aryl group or a substituted or unsubstituted heterocyclic group.

In certain embodiments, R₁₀ is a substituted or unsubstituted alkyl group (e.g., methyl).

In certain embodiments, R₁₁ is —H. Typically, when R₁₁ is —H, R₁₀ is a substituted or unsubstituted alkyl group (e.g., methyl).

In certain embodiments, R₁₀ and R₁₁ taken together with the atoms to which they are attached form a non-aromatic heterocyclic ring. Examples of such compounds are represented by Structural Formula (V):

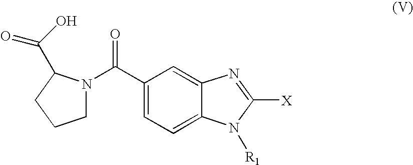

In certain embodiments, X in compounds represented by Structural Formulas (Ia), (IV) and (V) is —(CHR₂)ᵣ—NHCONR₄R₅. Typically, R₂ in such compounds is a substituted or unsubstituted alkyl group (e.g., methyl, ethyl, propyl, butyl, pentyl). Separately and in combination with these values of R₂, r is typically 1. Separately or in combination with these values of R₂ and r, R₄ is typically —H and R₅ is typically a substituted or unsubstituted alkyl (e.g., isopropyl) or alkenyl group.

In certain embodiments, X in compounds represented by Structural Formulas (Ia), (IV) and (V) is —SR₃ and R₃ is a substituted or unsubstituted alkyl group. Typically, R₃ in such compounds is a carboxy-substituted alkyl group (e.g., carboxymethyl), particularly when Ring A is unsubstituted.

In certain embodiments, X in compounds represented by Structural Formulas (Ia), (IV) and (V) is —(CH₂)ᵣ—NR₁SO₂—R₃ and R₃ is a substituted or unsubstituted aryl group.

In certain embodiments, X in compounds represented by Structural Formulas (Ia), (IV) and (V) is -arylene-R₂, wherein the arylene is phenylene (e.g., unsubstituted phenylene). R₂ in such compounds is typically a substituted or unsubstituted C₁-C₆ alkyl group or a halogen.

In another embodiment, compounds of the invention are represented by Structural Formula (I):

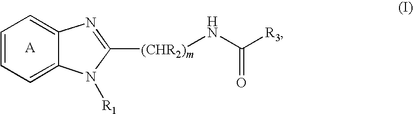

or a salt thereof where:

Ring A is optionally substituted;

R₁ is —H, a substituted or unsubstituted alkyl group, a substituted or unsubstituted alkenyl group, a substituted or unsubstituted aryl group or a substituted or unsubstituted heterocyclic group;

each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₃ is a substituted or unsubstituted alkyl group, a substituted or unsubstituted aryl group or a substituted or unsubstituted heterocyclic group;

R₄ and R₅ are independently —H, a substituted or unsubstituted alkyl group, a substituted or unsubstituted aryl group or a substituted or unsubstituted heterocyclic group;

m is an integer from 2 to 12; and

n is 1 or 2.

In certain embodiments, m is 2.

In certain embodiments, R₃ is a substituted or unsubstituted alkyl group, such as a carboxyalkyl group (e.g., carboxyethyl, 2,2-dimethylpropyl). In particular embodiments, R₃ is a substituted or unsubstituted alkyl group and m is 2.

In certain embodiments, R₁ is a substituted or unsubstituted alkyl group or a substituted or unsubstituted alkenyl group, such as ethyl, n-propyl, cyclopropylmethyl, 2-propenyl, benzyl and methoxyethyl. In particular embodiments, R₁ is a substituted or unsubstituted alkyl group or a substituted or unsubstituted alkenyl group when R₃ is a substituted or unsubstituted alkyl group and/or m is 2.

In certain embodiments, R₂ is —H or a substituted or unsubstituted aryl group, such as —H or a pyridyl group. In particular embodiments, R₂ is —H or a substituted or unsubstituted aryl group when R₁ is a substituted or unsubstituted alkyl group or a substituted or unsubstituted alkenyl group, R₃ is a substituted or unsubstituted alkyl group and/or m is 2. Preferred compounds are chosen such that R₂ is —H or a substituted or unsubstituted aryl group, R₁ is a substituted or unsubstituted alkyl group or a substituted or unsubstituted alkenyl group, R₃ is a substituted or unsubstituted alkyl group and m is 2.

In certain embodiments, R₃ is a substituted or unsubstituted alkyl group, such as a carboxyalkyl group (e.g., carboxyethyl, 2,2-dimethylpropyl). In particular embodiments, R₃ is a substituted or unsubstituted alkyl group and m is 2.

In certain embodiments, R₁ is a substituted or unsubstituted alkyl group or a substituted or unsubstituted alkenyl group, such as ethyl, n-propyl, cyclopropylmethyl, 2-propenyl, benzyl and methoxyethyl. In particular embodiments, R₁ is a substituted or unsubstituted alkyl group or a substituted or unsubstituted alkenyl group when R₃ is a substituted or unsubstituted alkyl group and/or m is 2.

In certain embodiments, R₂ is —H or a substituted or unsubstituted aryl group, such as —H or a pyridyl group. In particular embodiments, R₂ is —H or a substituted or unsubstituted aryl group when R₁ is a substituted or unsubstituted alkyl group or a substituted or unsubstituted alkenyl group, R₃ is a substituted or unsubstituted alkyl group and/or m is 2. Preferred compounds are chosen such that R₂ is —H or a substituted or unsubstituted aryl group, R₁ is a substituted or unsubstituted alkyl group or a substituted or unsubstituted alkenyl group, R₃ is a substituted or unsubstituted alkyl group and m is 2.

In another embodiment, [symbol] -modulating compounds of the invention are represented by Structural Formula (II):

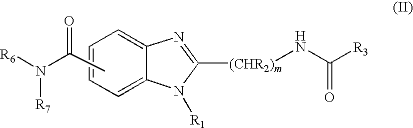

or a salt thereof, where:

R₁ is —H, a substituted or unsubstituted alkyl group, a substituted or unsubstituted aryl group or a substituted or unsubstituted heterocyclic group;

each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;

R₃ is a substituted or unsubstituted alkyl group, a substituted or unsubstituted aryl group or a substituted or unsubstituted heterocyclic group;

R₄ and R₅ are independently —H, a substituted or unsubstituted alkyl group, a substituted or unsubstituted aryl group or a substituted or unsubstituted heterocyclic group;

R₆ and R₇ are independently —H, a substituted or unsubstituted alkyl group, a substituted or unsubstituted aryl group or a substituted or unsubstituted heterocyclic group, or R₆ and R₇ taken together with the nitrogen atom to which they are attached form a heterocyclic ring;

m is an integer from 2 to 12; and

n is 1 or 2.

In certain embodiments, R₆ is a substituted alkyl group, such as a carboxyalkyl group (e.g., 1-carboxyethyl).

In certain embodiments, R₇ is —H. In particular embodiments, R₇ is —H and is a substituted alkyl group.

In certain embodiments, R₆ and R₇ taken together with the nitrogen atom to which they are attached form a heterocyclic ring. Suitable heterocyclic rings include substituted or unsubstituted piperazine and pyrrolidine, such as piperazine or 2-carboxypyrrolidine.

In certain embodiments, m is 2. In particular embodiments, m is 2 and R₆ and R₇ have the values indicated above.

In certain embodiments, R₃ is a substituted or unsubstituted alkyl group, such as a carboxyalkyl group (e.g., carboxyethyl, 2,2-dimethylpropyl). In particular embodiments, R₃ is a substituted or unsubstituted alkyl group when m is 2 and/or R₆ and R₇ have the values indicated above.

In certain embodiments, R₁ is a substituted or unsubstituted alkyl group or a substituted or unsubstituted alkenyl group, such as ethyl, n-propyl, cyclopropylmethyl, 2-propenyl, 2-propynyl, benzyl and methoxyethyl. In particular embodiments, R₁ is a substituted or unsubstituted alkyl group or a substituted or unsubstituted alkenyl group when R₃ is a substituted or unsubstituted alkyl group, R₆ and R₇ have the values indicated above and/or m is 2.

In certain embodiments, R₂ is —H or a substituted or unsubstituted aryl group, such as —H or a pyridyl group. In particular embodiments, R₂ is —H or a substituted or unsubstituted aryl group when R₁ is a substituted or unsubstituted alkyl group or a substituted or unsubstituted alkenyl group, R₃ is a substituted or unsubstituted alkyl group, R₆ and R₇ have the values indicated above and/or m is 2. Preferred compounds are chosen such that R₂ is —H or a substituted or unsubstituted aryl group, R₁ is a substituted or unsubstituted alkyl group or a substituted or unsubstituted alkenyl group, R₃ is a substituted or unsubstituted alkyl group, R₆ and R₇ have the values indicated above and m is 2.

One group of compounds of the invention encompassed by Structural Formula (II) is represented by Structural Formula (III):

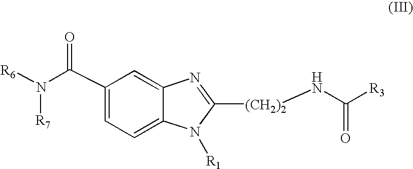

In certain embodiments, the compounds of the invention exclude those species disclosed in the Tables 3 and 4.

Novel compounds of the invention can also be used in the methods described above.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.
[symbol] modulating compounds of the invention having hydroxyl substituents, unless otherwise indicated, also include the related secondary metabolites, particularly sulfate, phosphate, acyl (e.g., acetyl, fatty acid acyl) and sugar (e.g., glucurondate, glucose) derivatives. In other words, substituent groups —OH also include —OSO3− M+, where M+ is a suitable cation (preferably H+, NH4+ or an alkali metal ion such as Na+ or K+) and sugars such as

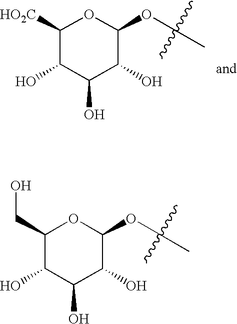

These groups are generally cleavable to —OH by hydrolysis or by metabolic (e.g., enzymatic) cleavage.
[symbol] modulating compounds of the invention advantageously modulate the level and/or activity of a protein.

Separately or in addition to the above properties, certain [symbol] modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight-chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A 5- to 7-membered ring includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent Examples of suitable substituents include —OH, halogen (−Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}); —NHCOR^{a}, —NRCOR^{a}, —NHCONH₂, —NHCONR^{a}H,

TABLE 3

| COMPOUND NO | STRUCTURE | ED₅₀ |
|---|---|---|
| 1 | 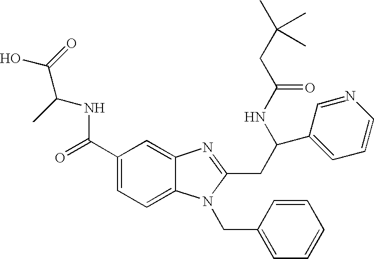 | B |
| 2 | 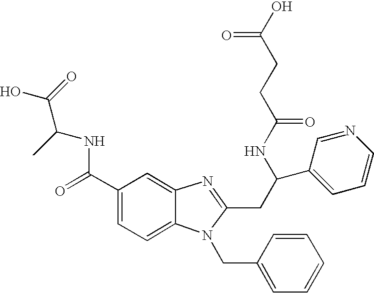 | B |
| 3 | 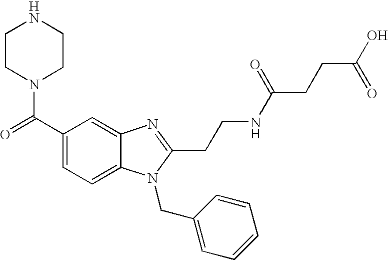 | A |
| 4 | 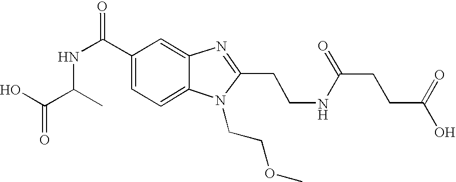 | B |
| 5 | 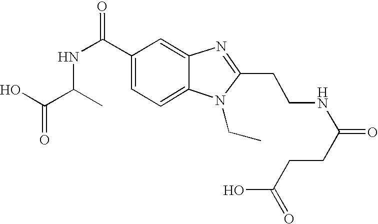 | B |
| 6 | 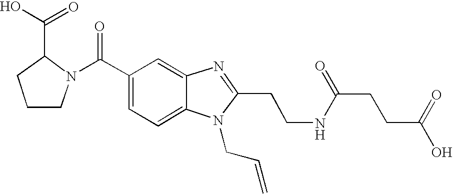 | A |
| 7 | 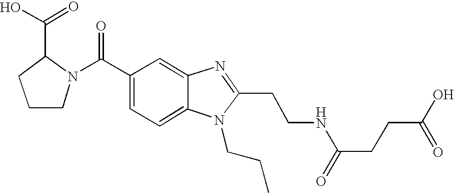 | A |
| 8 | 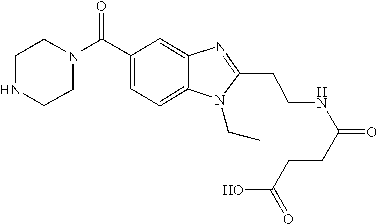 | C |
| 9 | 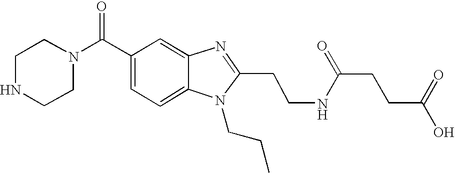 | B |
| 10 | 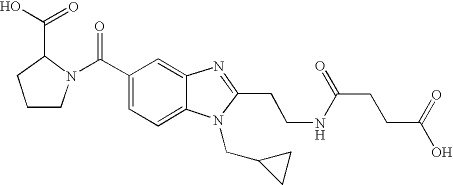 | A |
| 11 | 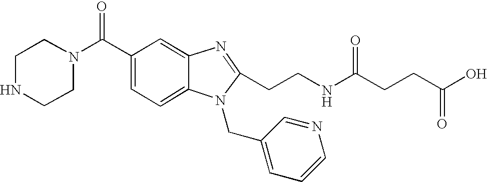 | A |
| 12 | 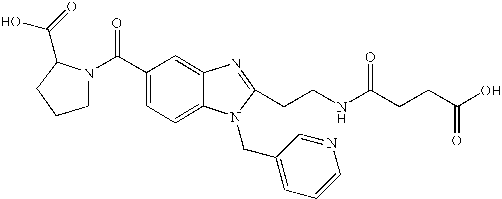 | A |
| 13 | 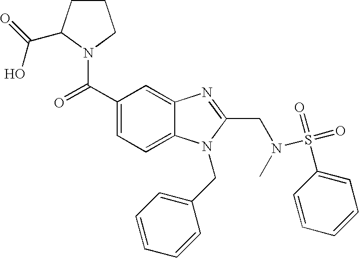 | B |
| 14 | 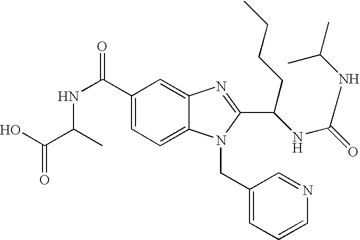 | B |

TABLE 4

| COMPOUND NO | STRUCTURE | ED₅₀ |
|---|---|---|
| 10 | 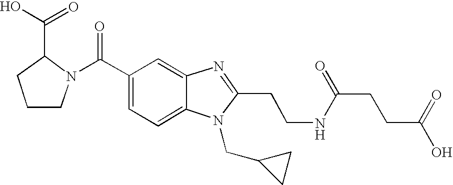 | D |
| 12 | 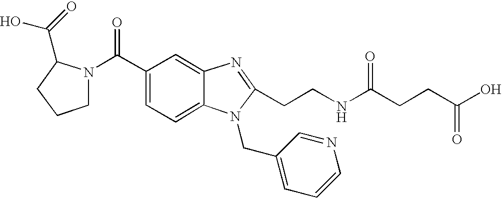 | D |
| 17 | 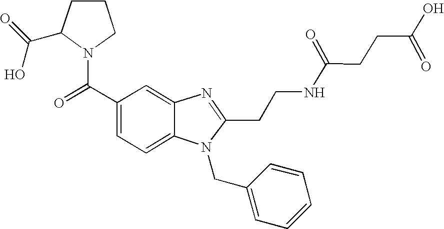 | A |
| 18 | 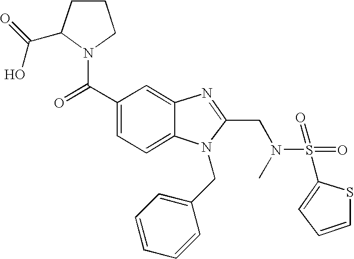 | C |
| 19 | 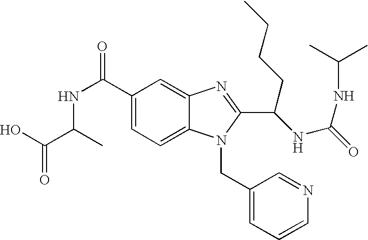 | A |

In one aspect, the invention provides novel [symbol] compounds for treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc.

In one embodiment, [symbol] compounds of the invention are represented by Structural Formula (Ia):

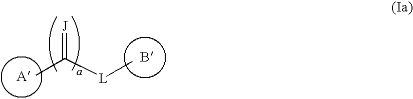

or a salt thereof, where:

Ring A′ is a 5- to 7-membered ring optionally fused to a second 5- to 7-membered ring, which is optionally substituted with one to three functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl;

Ring B′ is a 5- to 7-membered ring optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl;

J is O or S;

L is —C═C— or —NH—(CH₂)ₖ—;

R and R′ are independently —H, a substituted or unsubstituted alkyl group, a substituted or unsubstituted alkenyl group, a substituted or unsubstituted non-aromatic heterocyclic group or a substituted or unsubstituted aryl group;

a is 0 or 1;

k is an integer from 1 to 4; and

n is 1 or 2,

provided that Ring A′ and Ring B′ are not both phenyl, provided that at least one of Ring A′ and Ring B′ is substituted with at least one hydrogen bond donating group, and provided that the compound is not 4-((E)-2-(pyridin-4-yl)vinyl)phenol.

Preferably, one or both of Ring A′ and Ring B′ are aromatic, more preferably, both are aromatic. Suitable aromatic groups include, but are not limited to, pyridyl, phenyl, thienyl, furanyl, indolyl, pyrrolyl, imidazolyl, oxazolyl and thiazolyl. Particularly suitable aromatic groups are phenyl and pyridyl.

For one class of compounds encompassed by Structural Formula (I), a is 0. When a is 0, L is typically —CH═CH—.

For another class of compounds encompassed by Structural Formula (I), a is 1. When a is 1, J is typically 0. When a is 1 and J is 0, k is typically 1.

Typically, the hydrogen bond donating group is —OR, —OCOR, —OSO₃H, —COOH, —SH or —NHR. Preferably, the hydrogen bond donating group is —OR, —OCOR, or —OSO₃H. When the hydrogen bond donating group is —OR, the group is preferably hydrolyzable or metabolically cleavable to —OH (e.g., R is a sugar).

In another embodiment [symbol] compounds of the invention are represented by Structural Formula (I):

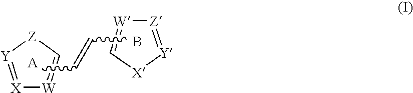

or a salt thereof, where:

W is CH or N;

X is CH or N;

Y is CH or N;

Z is S, O or NH;

W′ is CH or N;

X′ is CH or N;

Y′ is CH or N;

Z′ is S, O or NH;

R and R′ are independently —H, a substituted or unsubstituted alkyl group, a substituted or unsubstituted alkenyl group, a substituted or unsubstituted non-aromatic heterocyclic group or a substituted or unsubstituted aryl group;

n is 1 or 2;

Ring A is substituted with at least one hydrogen bond donating group and is optionally substituted with one to three functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl; and

Ring B is optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.

One group of [symbol] compounds encompassed by Structural Formula (I) is represented by Structural Formula (II):

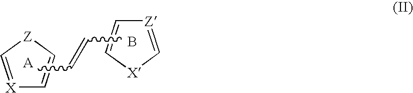

or a salt thereof.

Particular compounds represented by Structural Formula (II) include those where X is CH and Z is NH, O or S, or X is N and Z is S; and X′ is CH and Z′ is NH, O or S, or X is N and Z is S.

One group of [symbol] compounds encompassed by Structural Formula (II) is represented by Structural Formula (III):

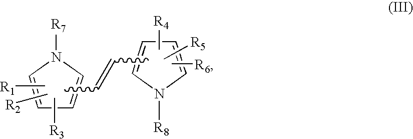

or a salt thereof; where:

R₁ is —OR, —OSO₃H, —SH, —NHR or —COOR;

R₂, R₃, R₄, R₅ and R₆ are independently —H, halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl or substituted or unsubstituted aryl; and

R₇ and R₈ are independently —H, a substituted or unsubstituted alkyl group, a substituted or unsubstituted alkenyl group or a substituted or unsubstituted aryl group.

A particular group of [symbol] compounds encompassed by Structural Formula (III) is represented by Structural Formula (IV):

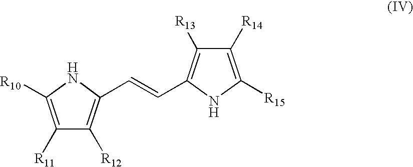

where:

R₁₀, R₁₁ and R₁₂ are independently —H, halogen, —OR, —CN, —CO₂R, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl or substituted or unsubstituted aryl, provided that at least one of R₁₀, R₁₁ and R₁₂ is —OH, —NHR, —SH or —COOR;

R₁₃, R₁₄ and R₁₅ are independently —H, halogen, —OR, —CN, —CO₂R, —OCO₂R, —C(O)NRR′, —OC(O)NRR′, —C(O)R, —COR, —SR, S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl or substituted or unsubstituted aryl.

In a preferred embodiment, at least one of R₁₀, R₁₁ and R₁₂ is —OR, —OCOR, or —OSO₃H, such as where two of these variables are —OR, —OCOR, or —OSO₃H. When the hydrogen bond donating group is —OR, the group is preferably hydrolyzable or metabolically cleavable to —OH (e.g., R is a sugar).

In another embodiment, at least one of R₁₀, R₁₁ and R₁₂ is a dihalomethyl group, such as a dihalomethyl (e.g., difluoromethyl, dichloromethyl) group.

When R₁₀, R₁₁ and R₁₂ have the values described above, in certain embodiments at least one of R₁₃, R₁₄ and R₁₅ is —OR, —OCOR, —OSO₃H, —NHR, —SH or —COOR, preferably —OR, —OCOR, or —OSO₃H. When the hydrogen bond donating group is —OR, the group is preferably hydrolyzable or metabolically cleavable to —OH (e.g., R is a sugar).

In another embodiment, [symbol] compounds of the invention are represented by Structural Formula (V):

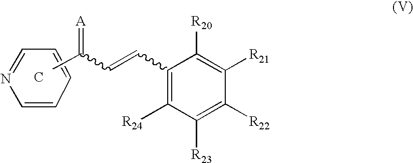

or a salt thereof, where:

A is O, NH or S;

R₂₀, R₂₁, R₂₂, R₂₃ and R₂₄ are independently —H, halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic or substituted or unsubstituted aryl;

R and R′ are independently —H, a substituted or unsubstituted alkyl group, a substituted or unsubstituted alkenyl group, a substituted or unsubstituted non-aromatic heterocyclic group or a substituted or unsubstituted aryl group;

n is 1 or 2; and

Ring C is optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.

One group of [symbol] compounds encompassed by Structural Formula (V) is represented by Structural Formula (VI):

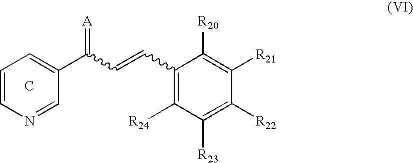

One group of [symbol] compounds encompassed by Structural Formula (VI) is represented by Structural Formula (VII):

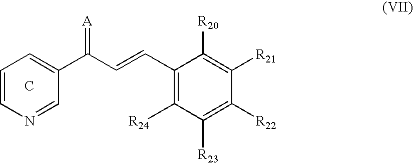

Typically, Ring C is unsubstituted. When Ring C is unsubstituted, A is typically O.

When Ring C and A have the values described above, one or two of R₂₀, R₂₁, R₂₂, R₂₃ and R₂₄ can be —OR, —OCOR, —OSO₃H, —NHR, —SH or —COOR, preferably —OR, —OCOR, or —OSO₃H, and the remainder of R₂₀, R₂₁, R₂₂, R₂₃ and R₂₄ can be —H. In another embodiment, one or two of R₂₀, R₂₁, R₂₂, R₂₃ and R₂₄ is a dihalomethyl group, preferably a difluoromethyl group. When the hydrogen bond donating group is —OR, the group is preferably hydrolyzable or metabolically cleavable to —OH (e.g., R is a sugar).

In one embodiment where compounds having the values of A, R₂, R₂₁, R₂₂, R₂₃ and R₂₄ described above and where Ring C is substituted or unsubstituted, one or two of R₂₁, R₂₂ and R₂₃ are —OR, —OCOR, or —OSO₃H. When the hydrogen bond donating group is —OR, the group is preferably hydrolyzable or metabolically cleavable to —OH (e.g., R is a sugar).

In a further embodiment, [symbol] compounds of the invention are represented by Structural Formula (VIII)

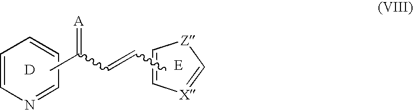

or a salt thereof, where:

A is O, NH or S;

X″ is CH or N;

Z″ is NH, O or S;

R and R′ are independently —H, a substituted or unsubstituted alkyl group, a substituted or unsubstituted alkenyl group, a substituted or unsubstituted non-aromatic heterocyclic group or a substituted or unsubstituted aryl group;

n is 1 or 2;

Ring D is optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl; and

Ring E is optionally substituted with one to three functional groups selected from the group consisting of halogen, —OR, —CN, —CO₂R, —OCO₂R, —OCOR, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.

Typically, A is O. When A is O, X″ can be CH.

One group of [symbol] compounds encompassed by Structural Formula (VIII) is represented by Structural Formula (IX):

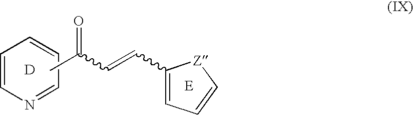

One group of [symbol] compounds encompassed by Structural Formula (IX) is represented by Structural Formula (X):

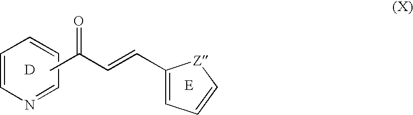

A particular group of [symbol] compounds encompassed by Structural Formula (X) is represented by Structural Formula (XI):

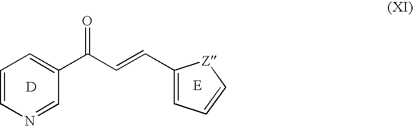

Typically, Z″ for compounds of Structural Formulae (VIII) (XI) is NH. When Z″ is NH, Ring D is preferably unsubstituted and Ring E is optionally substituted with one or two —OR, —OCOR, —OSO₃H, —NHR, —SH or —COOR groups, preferably one or two —OR, —OCOR, or —OSO₃H groups. When the hydrogen bond donating group is —OR, the group is preferably hydrolyzable or metabolically cleavable to —OH (e.g., R is a sugar).

In another group of compounds of the invention, Z″ and Ring D are as described above, and Ring E is substituted with one or two dihalomethyl groups. Preferably, the dihalomethyl group is a difluoromethyl group.

Further [symbol] compounds of the invention are represented by Structural Formula (XII):

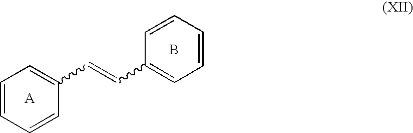

or a salt thereof where:

Ring A is substituted with at least one dihalomethyl group and at least one group capable of donating hydrogen bonds; and

Ring B is optionally substituted.

When Ring B is substituted, one or more of the substituents are preferably a group capable of donating hydrogen bonds.

For both Ring A and Ring B, typical hydrogen bond donating groups are —OR, —OCOR, —OSO₃H, —NHR, —SH and —COOR (where R is as defined above), preferably —OR, —OCOR, or —OSO₃H. When the hydrogen bond donating group is —OR, the group is preferably hydrolyzable or metabolically cleavable to —OH (e.g., R is a sugar).

Suitable dihalomethyl groups include dichloromethyl, dibromomethyl and difluoromethyl, preferably difluoromethyl.

A particular [symbol] compound encompassed by Structural Formula (XII) is represented by Structural Formula (XIII):

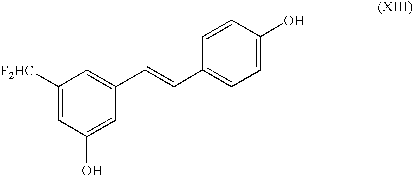

In one embodiment, [symbol] compounds of the invention are represented by Structural Formula (XIV):

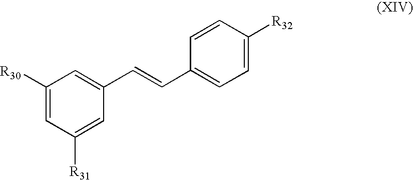

or a salt thereof, where:

R₃₀ is —OR_{z}, —OCH₃, —Cl, —OC₆H₅ or —CH₃;

R₃₁ is —H, —OR_{z}, —OCH₃, —F or —CH₃;

R₃₂ is —OR_{z}, —OCHF₂, —OCHCl₂, —OCHBr₂ or —OCH₃; and

R_{z} is —SO₃H, an acyl group (e.g., acetyl or the acyl group of a fatty acid) or a sugar, provided that R₃₂ is —OCHF₂, —OCHCl₂, —OCHBr₂ or —OCH₃ when R₃₀ and R₃₁ are both —OH.

Particular [symbol] compounds encompassed by Structural Formula (XIV) are represented by the following structural formulae:

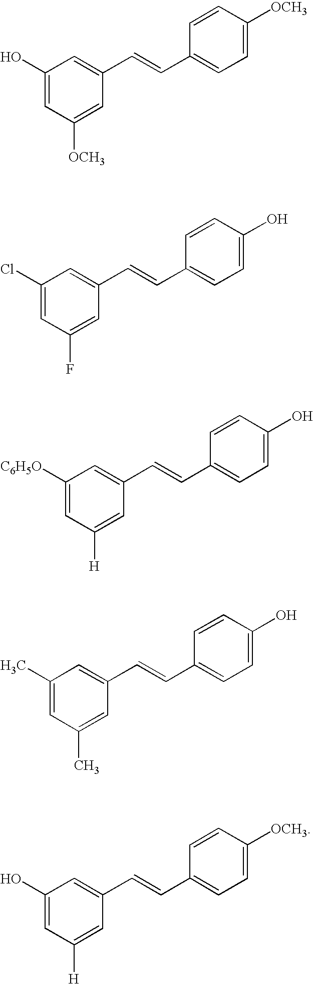

The hydroxyl groups of these compounds can be replaced with —OSO₃H or —OR_{z}, where R_{z} is an acyl group (e.g., acetyl or the acyl group of a fatty acid) or a naturally or non-naturally occurring sugar.

Additional [symbol] compounds encompassed by Structural Formula (XIV) are represented by the following formulae:

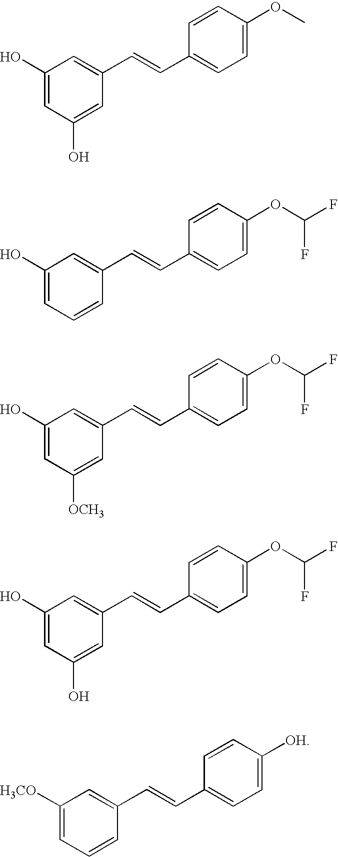

The hydroxyl groups of these compounds can be replaced with —OSO₃H or —OR_{z}, where R_{z} is an acyl group (e.g., acetyl or the acyl group of a fatty acid) or a naturally or non-naturally occurring sugar.

In another embodiment, [symbol] compounds of the invention are represented by Structural Formula (XV):

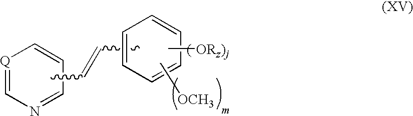

or a salt thereof, where:

j is 1 or 2;

m is 0 or 1;

Q is CH or N; and

R_{z}′ is —H, —SO₃H, acyl or a sugar, provided that the compound is not 4-((E)-2-(pyridin-4-yl)vinyl)phenol.

One group of [symbol] compounds encompassed by Structural Formula (XV) is represented by Structural Formula (XVI):

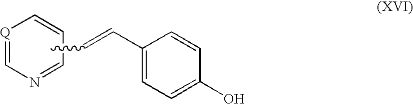

or a salt thereof, where Q is CH or N, provided that Q is N when the nitrogen atom is para to the double bond. The hydroxyl groups of these compounds can be replaced with —OSO₃H or —OR_{z}, where R_{z} is an acyl group (e.g., acetyl or the acyl group of a fatty acid) or a naturally or non-naturally occurring sugar.

Particular [symbol] compounds encompassed by Structural Formula (XVI) are represented by the following structural formulae:

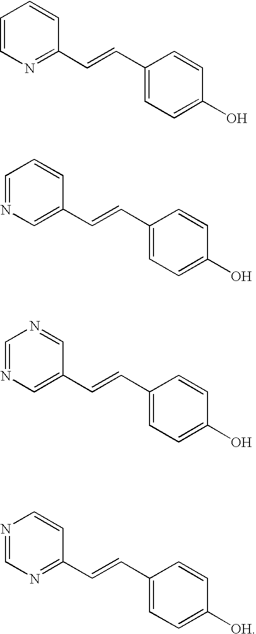

The hydroxyl groups or these compounds can be replaced with —OSO₃H or —OR_{z}, where R_{z} is an acyl group (e.g., acetyl or the acyl group of a fatty acid) or a naturally or non-naturally occurring sugar.

Another group of [symbol] compounds encompassed by Structural Formula (XV) is represented by Structural Formula (XVII):

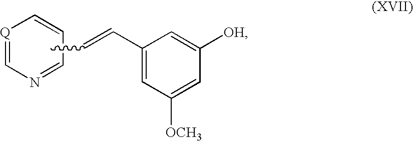

or a salt thereof, where Q is Cl or N. The hydroxyl groups of these compounds can be replaced with —OSO₃H or —OR_{z}, where R_{z} is an acyl group (e.g., acetyl or the acyl group of a fatty acid) or a naturally or non-naturally occurring sugar.

Particular [symbol] compounds encompassed by Structural Formula (XVII) are represented by the following structural formulae:

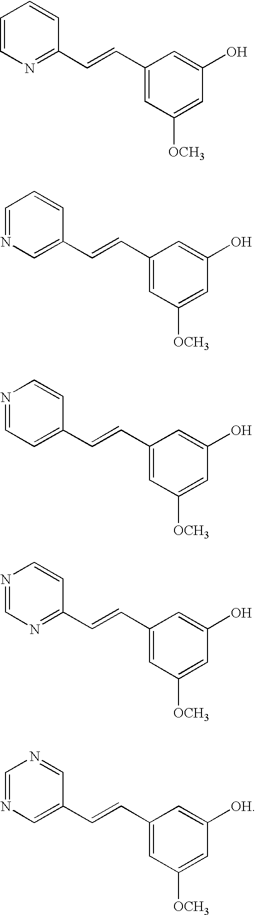

The hydroxyl groups of these compounds can be replaced with —OSO₃H or —OR_{z}, where R_{z} is an acyl group (e.g., acetyl or the acyl group of a fatty acid) or a naturally or non-naturally occurring sugar.

Other particular [symbol] compounds encompassed by Structural Formula (XV) are represented by the following:

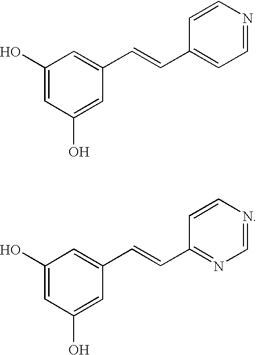

The hydroxyl groups of these compounds can be replaced with —OSO₃H or —OR_{z}, where R_{z} is an acyl group (e.g., acetyl or the acyl group of a fatty acid) or a naturally or non-naturally occurring sugar.

In yet another embodiment, [symbol] compounds of the invention are represented by Structural Formula (XVIII):

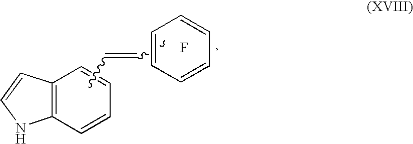

or a salt thereof; where:

Ring F is substituted with at least one hydrogen bond donating group and the compound is optionally substituted with one or more groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl;

R and R′ are independently —H, a substituted or unsubstituted alkyl group, a substituted or unsubstituted alkenyl group, a substituted or unsubstituted non-aromatic heterocyclic group or a substituted or unsubstituted aryl group; and

n is 1 or 2.

The hydrogen bonding donating group on Ring F is typically —OR, —OSO₃H, —SH, —NHR or —COOR, preferably —OR or —OSO₃H. When the hydrogen bond donating group is —OR, the group is preferably hydrolyzable or metabolically cleavable to —OH (e.g., R is a sugar or an acyl group).

Groups of [symbol] compounds encompassed by Structural Formula (XVIII) are represented by the following structural formulae:

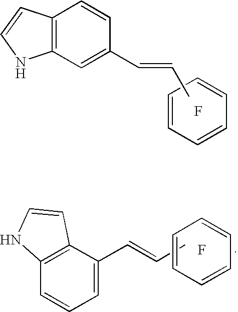

[symbol] compounds of the invention having hydroxyl substituents, unless otherwise indicated, also include the related secondary metabolites, particularly sulfate, acyl (e.g., acetyl, fatty acid acyl) and sugar (e.g., glucurondate, glucose) derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

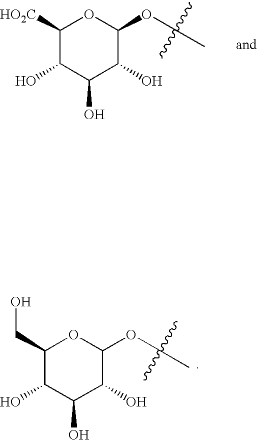

These groups are generally cleavable to —OH by hydrolysis or by metabolic (e.g., enzymatic) cleavage.

Separately or in addition to the above properties, certain sirtuin-activating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity,

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A 5- to 7-membered ring includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (−Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, (═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}, —NH—C(═NR^{c})—NH₂, —NH—C(═NR^{c})—NHR^{a}, —NH—C(—NR^{c})—N(R^{a}R^{b}, —NR^{d}H—C(═NH)—NH₂, —NR^{d}—C(═NH)—NHR^{a}, NR^{d}—C(═NH)—N(R^{a}R^{b}), NR^{d}—C(═NR^{c})—NH₂, —NR^{d}—C(═NR^{c})—NHR^{a}, —NR^{d}—C(═NR^{c})—N(R^{a}R^{b}), —NHNH₂—NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

In one embodiment, compounds for use in the methods described herein are represented by Structural Formula (I) or (II):

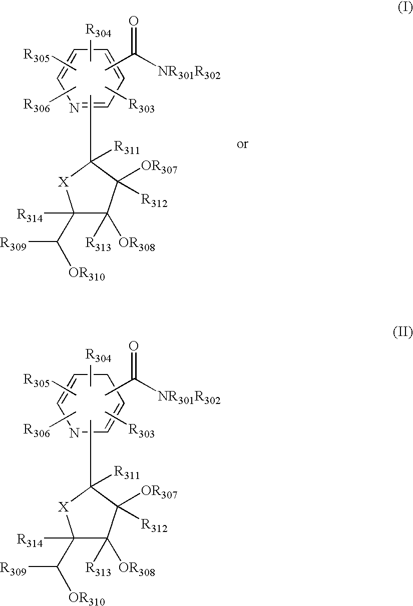

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

A group of suitable compounds encompassed by Structural Formulas (I) and (II) is represented by Structural Formulas (III) and (IV):

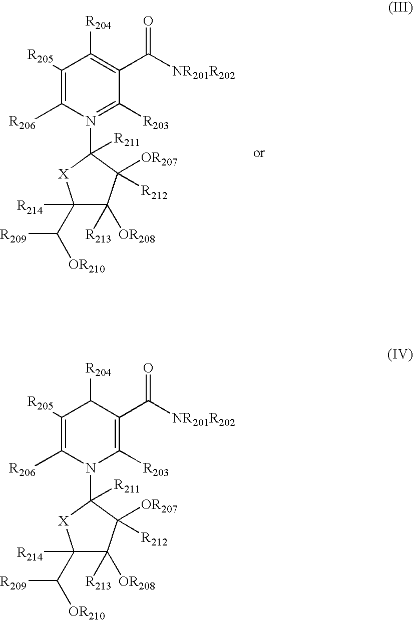

or a pharmaceutically acceptable salt thereof, where:

R₂₀₁ and R₂₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₂₀₁ and R₂₀₂ taken together with the atom to which they are attached form a substituted or unsubstituted non-aromatic heterocyclic group;

R₂₀₃, R₂₀₄, R₂₀₂ and R₂₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR,

Typically, for compounds represented by Structural Formulas (III) and (IV), R₂₀₃-R₂₀₆ are —H. In addition, R₂₀₉ and R₂₁₁-R₂₁₄ are typically —H. Particular compounds represented by Structural Formulas (III) and (IV) are selected such that R₂₀₃-R₂₀₀, R₂₀₉ and R₂₁₁-R₂₁₄ are all —H. For these compounds, R₂₀₄, R₂₀₇, R₂₀₈ and R₂₁₀ have the values described above.

R₂₀₁ and R₂₀₂ are typically —H or a substituted or unsubstituted alkyl group, more typically —H. In compounds having these values of R₂₀₁ and R₂₀₂, R₂₀₃-R₂₀₆, R₂₀₉ and R₂₁₁-R₂₁₄ typically have the values described above.

In certain methods of the invention, at least one of R₂₀₁-R₂₁₄ is not —H when X is O.

In certain methods of the invention, R₂₀₆ is not —H or —NH₂ when R₂₀₁-R₂₀₅ and R₂₀₇-R₂₁₄ are each —H.

In certain methods of the invention, the method does not include the reduction of a prodrug to an active agent by a NAD(P)H quinone reductase.

In certain methods of the invention, the subject being treated is not in need of an increase in nitric oxide bioactivity.

In certain methods of the invention, the method does not include the reduction of a prodrug to an active agent by a NAD(P)H quinone reductase and the subject being treated is not in need of an increase in nitric oxide bioactivity.

One embodiment of pharmaceutical compositions of the invention includes a pharmaceutically acceptable carrier or diluent and a compound represented by Structural Formula (V) or (VI):

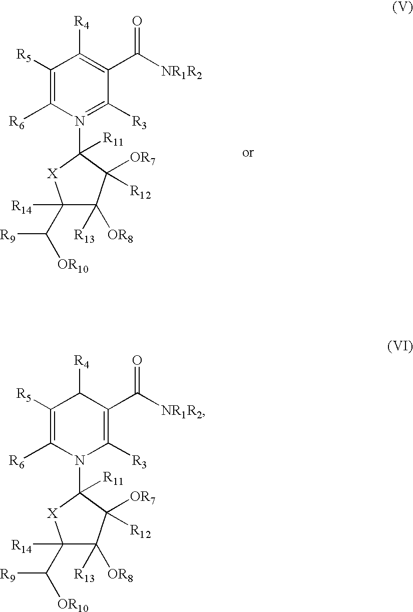

### —OSO₃H, —S(O)ₙR, —S(O)ₙOR, —S(O)ₙNRR′, —NRR′, —NRC(O)OR′, —NO₂ and —NRC(O)R′;

R₂₀₇, R₂₀₈ and R₂₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₂₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₂₁₁, R₂₁₂, R₂₁₃ and R₂₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2.

In a particular group of compounds represented by Structural Formula (II) or (IV), at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR: Typically, at least one of R₂₀₇, R₂₀₈ and R₂₁₀ is —C(O)R or —C(O)OR. More typically, at least one of R₂₀₇, R₂₀₈ and R₂₁₀ is —C(O)R. In such compounds, R is preferably a substituted or unsubstituted alkyl, particularly an unsubstituted alkyl group such as methyl or ethyl.

In another particular group of compounds represented by Structural Formula (III) or (IV), R₂₀₄ is a halogen (e.g., fluorine, bromine, chlorine) or hydrogen (including a deuterium and/or tritium isotope). Suitable compounds include those where at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR and R₂₀₄ is a halogen or hydrogen.

or a pharmaceutically acceptable salt thereof, wherein:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together with the atom to which they are attached form-a substituted or unsubstituted non-aromatic heterocyclic group, provided that when one of R₁ and R₂ is —H, the other is not an alkyl group substituted by —C(O CH₂ CH₃;

R₃, R₄ and R₅ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OC₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₆ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRC(O)OR′, —NO₂ and —NRC(O)R′;

R₇, R₈ and R₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2,

provided that R₁-R₁₄ are not each —H and that R₁-R₉ and R₁₁-R₁₄ are not each —H when R₁₀ is —C(O)C₆H₅.

In certain embodiments, R₁ is —H.

In certain embodiments, R₇, R₈ and R₁₀ are independently —H, —C(O)R or —C(O)OR, typically —H or —C(O)R such as —H or —C(O)CH₃. In particular embodiments, R₁ is —H and R₇, R₈ and R₁₀ are independently —H, —C(O)R or —C(O)OR.

In certain embodiments, R₉ is —H. In particular embodiments, R₉ is —H when R₁ is —H and/or R₇, R₈ and R₁₀ are independently —H, —C(O)R or —C(O)OR.

In certain embodiments, R₂ is —H. In particular embodiments, R₂ is —H when R₉ is —H, R₁ is —H and/or R₇, R₈ and R₁₀ are independently —H, —C(O)R or —C(O)OR. Typically, R₂ is —H when R₉ is —H, R₁ is —H and R₇, R₈ and R₁₀ are independently —H, —C(O)R or —C(O)OR.

In certain embodiments, R₄ is —H or a halogen, such as deuterium or fluorine.

In one embodiment, the invention is pharmaceutical composition comprising a pharmaceutically acceptable carrier or diluent and a compound represented by Structural Formula (VII) or (VIII):

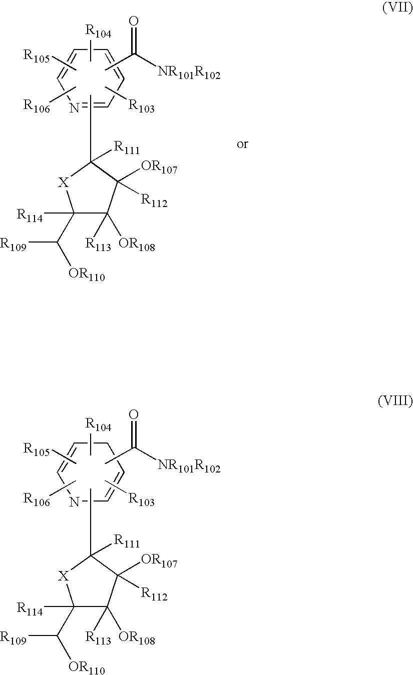

or a pharmaceutically acceptable salt thereof, wherein:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₁₀ and R₁₀₂ taken together with the atom to which they are attached form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NR, —C(S)R, —C(S)OR′ or —C(O)SR;

R₁₀₉ is selected from the group consisting of —H, a substituted or unsubstittited alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, the invention is a pharmaceutical composition comprising a pharmaceutically acceptable carrier or diluent and a compound represented by Structural Formula (IX) or (X):

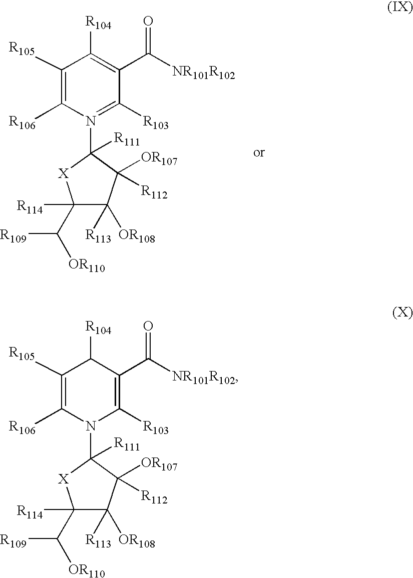

or a pharmaceutically acceptable salt thereof; where:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₁₀ and R₁₀₂ taken together with the atom to which they are attached form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCOR, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

For compounds represented by Structural Formulas (VI)-(X), typically at least one of R₁₀₇ and R₁₀₈ is —C(O)R, such as —C(O)CH₃. In particular embodiments, R₁₀₇, R₁₀₈ and R₁₁₀ are independently —H or —C(O)R (e.g., —C(O)CH₃).

In certain embodiments, such as when R₁₀₇, R₁₀₈ and R₁₁₀ have the values described above, R₁₀₁ and R₁₀₂ are each —H.

In certain embodiments, R₁₀₉ is —H.

In certain embodiments, R₁₀₃-R₁₀₆ are each —H.

In certain embodiments, R₁₁₁-R₁₁₄ are each —H.

In particular embodiments, R₁₀₇, R₁₀₈ and R₁₁₀ have the values described above and R₁₀₁-R₁₀₆, R₁₀₉ and R₁₁₁-R₁₁₄ are each —H.

In certain embodiments, R₁₀₄ is —H or a halogen, typically deuterium or fluorine. The remaining values are as described above.

Compounds included in pharmaceutical compositions of the invention can also be used in the methods described above.

For compounds represented by Structural Formula (XI) or (XII):

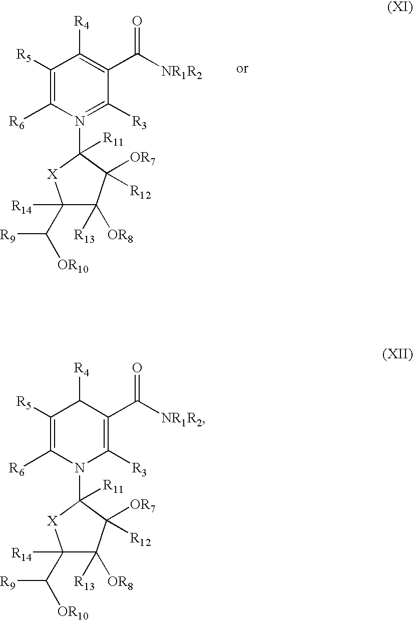

R₄ in certain embodiments is —H (e.g., deuterium, tritium) or a halogen (e.g., fluorine, bromine, chlorine).

In embodiments of the invention where R₁-R₆ can each be —H, they typically are each —H. In embodiments of the invention where one of R₁-R₆ is not —H, typically the remaining values are each —H and the non-H value is a substituted or unsubstituted alkyl group or a halogen (R₁ and R₂ are typically a substituted or unsubstituted alkyl group).

In certain embodiments, R₁₁-R₁₄ are each —H. When R₁₁-R₁₄ are each —H, R₁-R₆ typically have the values described above.

In certain embodiments, R₉ is —H. When R₉ is —H, typically R₁₁-R₁₄ are each —H and R₁-R₆ have the values described above.

Novel compounds of the invention can also be used in the methods described above.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.
[symbol] modulating compounds also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻ M⁺, where M⁺ is a suitable cation (preferably H⁺, NH₄⁺ or an alkali metal ion such as Na⁺ or K⁺) and sugars such as

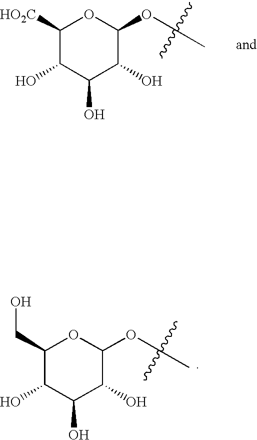

These groups are generally cleavable to —OH by hydrolysis or by metabolic (e.g., enzymatic) cleavage.

In certain embodiments, compounds having Structural Formula A are excluded from the compounds, pharmaceutical compositions and/or methods of the invention:

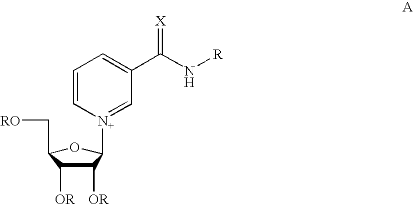

wherein

R represents independently for each occurrence H, acetyl, benzoyl, acyl, phosphate, sulfate, (alkyoxy)methyl, triarylmethyl, (trialkyl)silyl, (dialkyl)(aryl)silyl, (alkyl)(diaryl)silyl, or (triaryl)silyl; and

X represents O or S.

Compounds that Modulate HAT

In one embodiment, [symbol] compounds of the invention are represented by Structural Formula I:

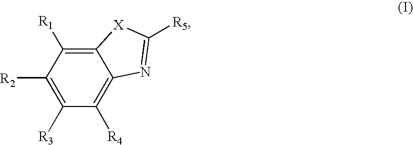

wherein:

one of R₁, R₂, R₃ or R₄ is selected from a 5- to 6-membered heteroaryl comprising 1 to 3 heteroatoms independently selected from N, O or S, wherein said heteroaryl is optionally benzofused or optionally fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

the others of R₁, R₂, R₃ and R₄ are independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and —R₅ is an optionally substituted aryl or an optionally substituted heteroaryl.

In certain embodiments, when X is NR₆, R₆ is hydrogen, and R₂ or R₃ is 1,2,4-oxadiazole-3-yl, pyridin-4-yl, or furan-2-yl, then R₅ is not phenyl substituted with an acylamino group.

In certain embodiments, when X is NR₆, R₆ is H or alkoxymethyl, and R₂ or R₃ is substituted or unsubstituted 1H-imidazo[4,5-b]pyridin-2-yl, substituted or unsubstituted 1H-benzimidazol-2-yl, or substituted or unsubstituted oxazolo[5,4-b]pyridin-2-yl, then R₅ is not phenyl monosubstituted with hydroxy or methoxy.

In certain embodiments, when X is NR₆, R₆ is H or alkoxymethyl, and R₂ or R₃ is benzo[b]thienyl, then R₅ is not 2H-indazol-3-yl.

In certain embodiments, when X is NR₆, R₆ is H or alkoxymethyl and R₂ or R₃ is 1H-imidazo[2,1-b]thiazol-3-yl, then R₅ is not unsubstituted pyridin-4-yl.

In certain embodiments, when X is NR₆ and R₂ or R₃ is 1,2,4-oxadiazol-3-yl, then R₅ is not 2-chloro-5-nitrophenyl.

In certain embodiments, when X is NR₆ and R₂ or R₃ is thien-3-yl or furan-2-yl, then R₅ is not unsubstituted 2H-indazol-3-yl.

In certain embodiments, when X is NR₆ and R₂ or R₃ is imidazol-4-yl or thiazolyl, then R₅ is not unsubstituted thiazol-4-yl.

In certain embodiments, when X is NR₆ and R₂ or R₃ is pyridazin-3-yl, then R₅ is not 4-methylphenyl or 4-methoxyphenyl.

In certain embodiments, when X is NR₆ and R₂ or R₃ is pyridinyl, then R₅ is not unsubstituted phenyl, 4-fluorophenyl, 4-methylphenyl, 4-(1,1-dimethylethyl)phenyl, or 4-trifluoromethylphenyl.

In certain embodiments, when X is S and R₂ or R₃ is substituted or unsubstituted benzothiazol-2-yl, then R₅ is not unsubstituted phenyl, 2-methyl substituted phenyl, 4-nitrophenyl, 4-amino substituted phenyl, 3- or 4-methylaminophenyl, 4-thiophenyl, 4-hydroxyphenyl, 4-(sulfonatomethyl)aminophenyl, 2-methyl substituted benzthiazol-6-yl, 2,3-dimethylbenzthiazol-6-yl, 4-amino-3-sulfonatophenyl, 4-amino-3-iodophenyl, 4-(4-acetamidophenylsulfamoyl)phenyl, or 4-(4-dimethylaminophenyl)iminophenyl.

In certain embodiments, when X is S and R₂ is substituted or unsubstituted pyrimidinyl, then R₅ is not phenyl substituted with an octyloxy-, propoxypropoxy-, glycine- or hexanoic acid-containing substituent and optionally one or more additional substituents.

In certain embodiments, when X is O and R₄ is substituted or unsubstituted benzoxazol-2-yl, then R₅ is not phenyl, 2-hydroxyphenyl, 2-methoxyphenyl, 2-phenylmethoxyphenyl, 2-hydroxy-3-phenylmethylphenyl, or 2-hydroxy-6-methylphenyl.

In certain embodiments, when X is O and R₄ is substituted or unsubstituted 1H-benzimidazol-2-yl, then R₅ is not 2-hydroxyphenyl.

In certain embodiments, when X is O and one of R₂ or R₃ is substituted or unsubstituted 1H-benzimidazol-2-yl, substituted or unsubstituted oxazolo[5,4-b]pyridin-2-yl or substituted or unsubstituted 1H-imidazo[4,5-b]pyridin-2-yl, then R₅ is not 4-hydroxyphenyl, 4-methoxyphenyl, 4-N,N-bis-(2-chloroethyl)aminophenyl, or naphthalen-4-yl.

In certain embodiments, when X is O and R₃ is 1-methyl-1H-pyrrol-2-yl, then R₅ is not 3-chloro-4-(5-methoxyfuran-2-yl)ethenylphenyl or 3-chloro-4-(5-methoxythien-2-yl)ethenylphenyl.

In certain embodiments, when X is O and R₂ is benzo[b]thien-2-yl, then R₅ is not 2-methoxy-5-aminophenyl, 3-amino-4-methoxyphenyl, 2-propylamino-5-aminophenyl, 2-methoxy-5-nitrophenyl, 4-methoxy-3-nitrophenyl, or 5-nitro-2-propylaminophenyl.

In certain embodiments, when X is O and one of R₂ or R₃ is benzofuran-2-yl, then R₅ is not 2-methoxy-5-aminophenyl, 2-propylamino-5-aminophenyl, 2-fluoro-5-nitrophenyl, 2-methoxy-5-nitrophenyl, 4-methoxy-3-nitrophenyl, or 5-nitro-2-propylaminophenyl.

In certain embodiments, when X is O and one of R₂ or R₃ is substituted or unsubstituted benzoxazol-2-yl, then R₅ is not 4-hydroxyphenyl, 4-styrylphenyl, 4-tert-butylphenyl, or 5-phenylthien-2-yl.

In certain embodiments, when X is O and R₁ is substituted or unsubstituted benzoxazol-2-yl, then R₅ is not 2-methoxyphenyl.

In certain embodiments, when X is O and one of R₂, R₃ or R₄ is furan-2-yl, then R₅ is not substituted or unsubstituted phenyl, benzo[b]thien-2-yl, benzoxazol-2-yl, naphthalen-2-yl, benzofuran-2-yl, quinolin-6-yl, or 5-methylthien-2-yl.

In certain embodiments, when X is O and R₃ is pyridin-3-yl, then R₅ is not 3-methoxyphenyl.

In certain embodiments, when X is O and one of R₂ or R₃ is substituted or unsubstituted pyrimidin-5-yl, then R₅ is not phenyl.

In certain embodiments, when X is O and R₃ is thiazol-2-yl, then R₅ is not thiazol-4-yl.

In certain embodiments, when X is O and one of R₂ or R₃ is substituted or unsubstituted thien-2-yl, then R₅ is not 3-amino-4-methoxyphenyl, 2-hydroxyphenyl, 3-[5-carboxylic acid-2,3-dihydro-1,3-dioxo-1H-isoindol-1-yl]-4-methoxyphenyl or 3-nitro-4-methoxyphenyl.

In another embodiment, [symbol] compounds of the invention are represented by Structural Formula II:

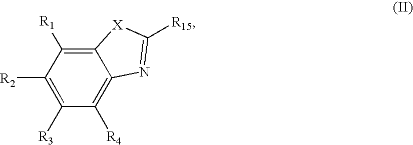

wherein:

one of R₂ or R₃ is selected from a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₁, R₄, and the other of R₂ or R₃ are each independently selected from hydrogen, a solubilizing group, or a C₁-C₄ straight or branched alkyl, wherein at least two of R₁, R₂, R₃ and R₄ are hydrogen;

X is selected from O, S or NR₆, wherein R₆ is selected from hydrogen, a solubilizing group, or a C₁-C₄ straight or branched alkyl; and

R₁₅ is an optionally substituted heteroaryl or an optionally substituted naphthalenyl.

In certain embodiments, when X is NR₆, R₆ is hydrogen, and R₂ or R₃ is 1,2,4-oxadiazole-3-yl, pyridin-4-yl, or furan-2-yl, then R₅ is not phenyl substituted with an acylamino group.

In certain embodiments, when X is NR₆ and R₂ or R₃ is benzo[b]thienyl, then R₅ is not 2H-indazol-3-yl.

In certain embodiments, when X is NR₆ and R₂ or R₃ is 1H-imidazo[2,1-b]thiazol-3-yl, then R₅ is not unsubstituted pyridin-4-yl.

In certain embodiments, when X is NR₆ and R₂ or R₃ is thien-3-yl or furan-2-yl, then R₅ is not unsubstituted 2H-indazol-3-yl.

In certain embodiments, when X is NR₆ and R₂ or R₃ is imidazol-4-yl or thiazolyl, then R₅ is not unsubstituted thiazol-4-yl.

In certain embodiments, when X is S and R₂ is benzothiazol-2-yl, then R₅ is not 2-methyl substituted benzthiazol-6-yl.

In certain embodiments, when X is O and one of R₂ or R₃ is benzoxazol-2-yl, then R₅ is not 5-phenylthien-2-yl.

In certain embodiments, when X is O and R₃ is thiazol-2-yl, then R₅ is not thiazol-4-yl.

In certain embodiments, when X is O and one of R₂ or R₃ is furan-2-yl, then R₅ is not unsubstituted benzo[b]thien-2-yl, unsubstituted benzoxazol-2-yl, or unsubstituted naphthalen-2-yl.

In another embodiment, [symbol] compounds of the invention are represented by Structural Formula III:

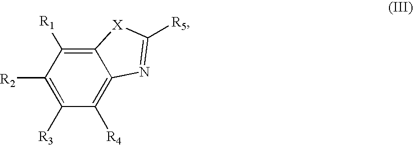

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, when X is O and R₄ is benzoxazol-2-yl, then R₅ is not unsubstituted phenyl or, 2-hydroxy, 2-methoxy or 2-phenylmethoxy substituted phenyl.

In certain embodiments, when X is O and R₄ is benzimidazole-2-yl, then R₅ is not 2-hydroxyphenyl.

In certain embodiments, when X is O and R₁ is benzoxazol-2-yl, then R₅ is not 2-methoxyphenyl.

In certain embodiments, one of R₁-R₄ is selected from:

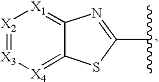

or

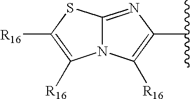

wherein:

X₁, X₂, X₃, and X₄ are each independently selected from CR₁₆ or N; and

two or three occurrences of R₁₆ are H and the other(s) is a substituent selected from a solubilizing group, or a C₁-C₄ straight or branched alkyl.

In certain embodiments, R₅ is selected from phenyl, 1H-pyrrolyl, pyridinyl, quinolinyl, quinoxalinyl, thienyl, or furanyl, wherein R₅ is optionally substituted.

In certain embodiments, R₅ is selected from 3,4-dimethoxyphenyl, 4-dimethylaminophenyl, 4-methoxyphenyl, 4-morpholinophenyl, or 3,4-dioxymethylenephenyl.

In another embodiment, [symbol] compounds of the invention are represented by Structural Formula IIIa:

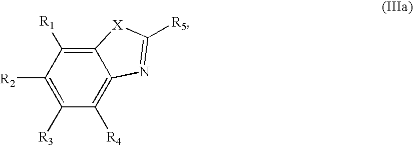

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, one of R₁-R₄ is selected from:

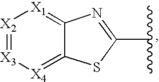

or

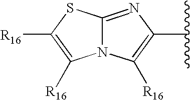

wherein:

X₁, X₂, X₃, and X₄ are each independently selected from CR₁₆ or N; and

two or three occurrences of R₆ are H and the other(s) is a substituent selected from a solubilizing group, or a C₁-C₄ straight or branched alkyl.

In certain embodiments, R₅ is selected from phenyl, 1H-pyrrolyl, pyridinyl, quinolinyl, quinoxalinyl, thienyl, or furanyl, wherein R₅ is optionally substituted.

In certain embodiments, R₅ is selected from 3,4-dimethoxyphenyl, 4-dimethylaminophenyl, 4-methoxyphenyl, 4-morpholinophenyl, or 3,4-dioxymethylenephenyl.

In another embodiment, [symbol] compounds of the invention are represented by Structural Formula IIIb:

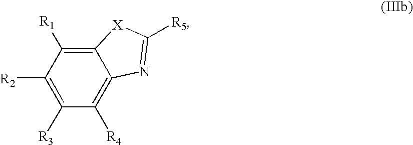

wherein:

one of R₁ or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein when said heteroaryl is benzofused, said heteroaryl comprises a S atom, and wherein said heteroaryl is optionally substituted on a single carbon ring atom with a substituent selected from a solubilizing group, or a C₁-C₄ straight or branched alkyl;

R₂, R₃ and the other of R₁ or R₄ are independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, one of R₁-R₄ is selected from:

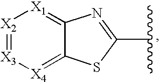

or

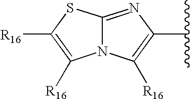

wherein:

X₁, X₂, X₃, and X₄ are each independently selected from CR₁₆ or N; and

two or three occurrences of R₁₆ are H and the other(s) is a substituent selected from a solubilizing group, or a C₁-C₄ straight or branched alkyl.

In certain embodiments, R₅ is selected from phenyl, 1H-pyrrolyl, pyridinyl, quinolinyl, quinoxalinyl, thienyl, or furanyl, wherein R₅ is optionally substituted.

In certain embodiments, R₅ is selected from 3,4-dimethoxyphenyl, 4-dimethylaminophenyl, 4-methoxyphenyl, 4-morpholinophenyl, or 3,4-dioxymethylenephenyl.

In another embodiment [symbol] compounds of the invention are represented by Structural Formula IV:

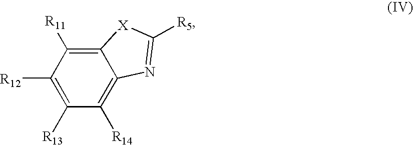

wherein:

one of R₁₁-R₁₄ is an 8- to 10-membered bicyclic heteroaryl comprising at least one heteroatom selected from N, O or S in each ring, wherein at least one of said heteroatoms in the bicyclic heteroaryl is an S atom, wherein said heteroaryl is bound to the rest of the compound via a carbon ring atom, and wherein said heteroaryl is optionally substituted on a single carbon ring atom with a substituent selected from a solubilizing group, or a C₁-C₄ straight or branched alkyl;

and the remainder of R₁₁-R₁₄ are each independently selected from hydrogen, a solubilizing group, or a C₁-C₄ straight or branched alkyl, wherein at least two of R₁₁, R₁₂, R₁₃ or R₁₄ are hydrogen;

X is selected from O, S or NR₆, wherein R₆ is selected from hydrogen, a solubilizing group, or a C₁-C₄ straight or branched alkyl; and

R₅ is an optionally substituted aryl or heteroaryl.

In certain embodiments, when R₅ is 4-pyridyl, R₅ is a substituted 4-pyridyl. In certain such embodiments, the 4-pyridyl may be substituted with halogen, hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl. In certain embodiments, the 4-pyridyl may be substituted with hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl.

In certain embodiments, X is NR₆.

In another embodiment, [symbol] compounds of the invention are represented by Structural Formula IVa:

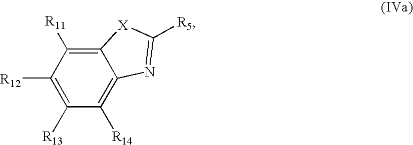

wherein:

one o R₁₂ or R₁₃ is an 8- to 10-membered bicyclic heteroaryl comprising at least one heteroatom selected from N, O or S in each ring, wherein at least one of said heteroatoms in the bicyclic heteroaryl is an S atom, wherein said heteroaryl is bound to the rest of the compound via a carbon ring atom, and wherein said heteroaryl is optionally substituted on a single carbon ring atom with a substituent selected from a solubilizing group, or a C₁-C₄ straight or branched alkyl;

R₁₁, R₁₄ and the other of R₁₂ or R₁₃ are each independently selected from hydrogen, a solubilizing group, or a C₁-C₄ straight or branched alkyl, wherein at least two of R₁₁, R₁₂, R₁₃ or R₁₄ are hydrogen;

X is selected from O, S or NR₆, wherein R₆ is selected from hydrogen, a solubilizing group, or a C₁-C₄ straight or branched alkyl; and

R₅ is an optionally substituted phenyl.

In certain embodiments, X is NR₆.

In another embodiment, [symbol] compounds of the invention are represented by Structural Formula V:

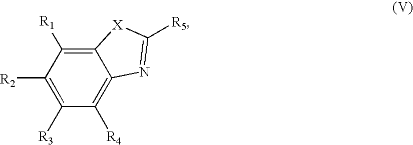

wherein:

one of R₂, or R₃ or R₄ is selected from

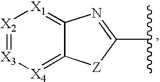

or

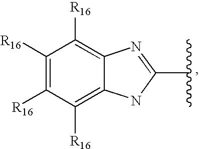

wherein:
one of X₁, X₂, X₃ or X₄ is N and the others are each CR₁₆;
Z is selected from NH, or O; and
each R₁₆ is independently selected from hydrogen, a solubilizing group, or a C₁-C₄ straight or branched alkyl, wherein no more than one R₁₆ is selected from a solubilizing group, or a C₁-C₄ straight or branched alkyl;

R₁, R₄ and the other of R₂ and R₃ are each independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted phenyl.

In certain embodiments, when X is NR₆ and R₂ or R₃ is 1H-imidazo[4,5-b]pyridin-2-yl, 1H-benzimidazol-2-yl, or oxazolo[5,4-b]pyridin-2-yl, and R₅ is phenyl substituted with hydroxy or methoxy, then there is at least one additional substituent. In certain such embodiments, the at least one additional substituent is selected from halogen, hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl. In certain embodiments, the at least one additional substituent is selected from hydroxyl, carbonyl (such as carboxyl, alkoxycarbonyl, formyl, or acyl), thiocarbonyl (such as thioester, thioacetate, or thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aryl, heteroaryl, cycloalkyl, or C₂-C₁₀ alkyl.

In certain embodiments, when X is O and R₄ is 1H-benzimidazole-2-yl, then R₅ is not 2-hydroxyphenyl.

In certain embodiments, when X is O and one of R₂ or R₃ is 1H-benzimidazol-2-yl, oxazolo[5,4-b]pyridin-2-yl or 1H-imidazo[4,5-b]pyridin-2-yl, then R₅ is not 4-hydroxyphenyl, 4-methoxyphenyl, or 4-N,N-bis(2-chloroethyl)aminophenyl.

In another embodiment, [symbol] compounds of the invention are represented by Structural Formula VI:

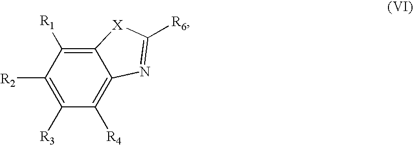

wherein:

one of R₂ or R₃ is selected from

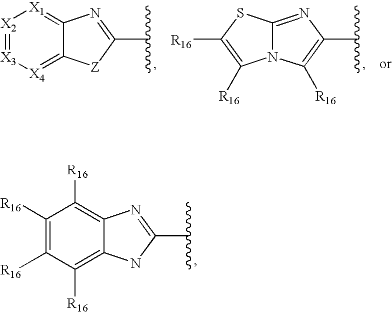

wherein:
one of X₁, X₂, X₃ or X₄ is N and the others are each CR₁₆;
Z is selected from NH, S or O; and
each R₁₆ is independently selected from hydrogen, a solubilizing group, or a C₁-C₄ straight or branched alkyl, wherein no more than one R₁₆ is selected from a solubilizing group, or a C₁-C₄ straight or branched alkyl;

R₁, R₄, and the other of R₂ and R₃ are each independently selected from hydrogen, a solubilizing group, or a C₁-C₄ straight or branched alkyl, wherein at least two of R₁, R₂, R₃ or R₄ are hydrogen;

X is S; and

R₅ is an optionally substituted aryl or an optionally substituted heteroaryl.

In certain embodiments, X is NR₆.

In certain embodiments, R₅ is selected from phenyl, 1H-pyrrolyl, pyridinyl, quinolinyl, quinoxalinyl, thienyl, or furanyl; and wherein R₅ is optionally substituted.

In certain embodiments, R₅ is selected from 5-methylfuran-2-yl, unsubstituted pyridin-4-yl, or unsubstituted thien-2-yl.

In certain embodiments, R₅ is selected from 1-methyl-1H-pyrrol-2-yl, 3,4-dimethoxyphenyl, 3,5-dimethoxyphenyl, 4-methoxyphenyl, 2,4,6-trimethylphenyl, 3-hydroxyphenyl, 2-fluoro-5-methylphenyl, unsubstituted phenyl, 4-dimethylaminophenyl, 3,4-dioxymethylenephenyl, 4-carboxyphenyl, unsubstituted pyridin-3-yl, unsubstituted pyridin-4-yl, unsubstituted quinolinyl, unsubstituted quinoxalinyl, or unsubstituted thien-2-yl.

In another embodiment, [symbol] compounds of the invention are represented by Structural Formula VIa:

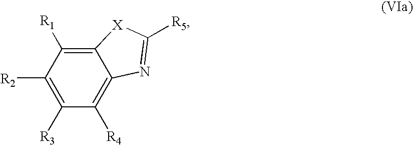

wherein:

one of R₁-R₄ is selected from

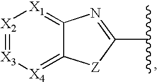

or

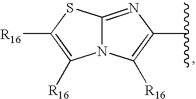

wherein:
one of X₁, X₂, X₃ or X₄ is N and the others are each CR₁₆;
Z is S; and
each R₁₆ is independently selected from hydrogen, a solubilizing group, or a C₁-C₄ straight or branched alkyl, wherein no more than one R₁₆ is selected from a solubilizing group, or a C₁-C₄ straight or branched alkyl;

the remainder of R₁-R₄ are each independently selected from hydrogen, a solubilizing group, or a C₁-C₄ straight or branched alkyl, wherein at least two of R₁, R₂, R₃ or R₄ are hydrogen;

X is selected from O, S or NR₆, wherein R₆ is selected from hydrogen, a solubilizing group, or a C₁-C₄ straight or branched alkyl; and

R₅ is an optionally substituted aryl or heteroaryl.

In certain embodiments, X is NR₆.

In certain embodiments, R₅ is selected from phenyl, 1H-pyrrolyl, pyridinyl, quinolinyl, quinoxalinyl, thienyl, or furanyl; and wherein R₅ is optionally substituted.

In certain embodiments, R₅ is selected from 5-methylfuran-2-yl, unsubstituted pyridin-4-yl, or unsubstituted thien-2-yl.

In certain embodiments, R₅ is selected from 1-methyl-1H-pyrrol-2-yl, 3,4-dimethoxyphenyl, 3,5-dimethoxyphenyl, 4-methoxyphenyl, 2,4,6-trimethylphenyl, 3-hydroxyphenyl, 2-fluoro-5-methylphenyl, unsubstituted phenyl, 4-dimethylaminophenyl, 3,4-dioxymethylenephenyl, 4-carboxyphenyl, unsubstituted pyridin-3-yl, unsubstituted pyridin-4-yl, unsubstituted quinolinyl, unsubstituted quinoxalinyl, or unsubstituted thien-2-yl.

In another embodiment, [symbol] compounds of the invention are represented by Structural Formula VII:

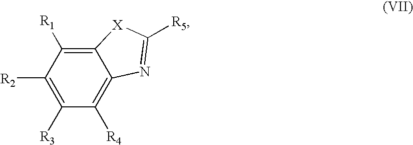

wherein:

one of R₁, or R₄ is a 5- to 6-membered heteroaryl comprising 1 to 3 heteroatoms independently selected from N, O or S, wherein said heteroaryl is optionally benzofused or fused to a second 5- to 6-membered heteroaryl comprising 1 to 3 heteroatoms independently selected from N, O or S and is bound to the rest of the compound via a carbon ring atom, wherein said heteroaryl is optionally substituted on a single carbon ring atom with a substituent selected from a solubilizing group, or a C₁-C₄ straight or branched alkyl;

R₂, R₃ and the other of R₁, or R₄ are independently selected from hydrogen, a solubilizing group, or a C₁-C₄ straight or branched alkyl, wherein at least two of R₁, R₂, R₃ or R₄ are hydrogen;

X is selected from S or NR₆, wherein R₆ is selected from hydrogen, a solubilizing group, or a C₁-C₄ straight or branched alkyl; and

R₅ is an optionally substituted aryl or an optionally substituted heteroaryl.

In certain embodiments, X is NR₆.

In certain embodiments one of R₁ or R₄ is selected from:

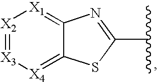

or

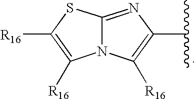

In certain embodiments, R₅ is selected from phenyl, 1H-pyrrolyl, pyridinyl, quinolinyl, quinoxalinyl, thienyl, or furanyl; and wherein R₅ is optionally substituted.

In certain embodiments of formulae I-VII above, the solubilizing group is selected from

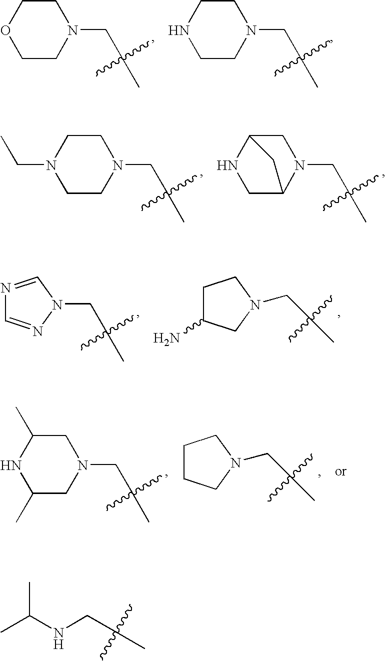

In certain embodiments of formulae I-VII above, R₅ or R₁₅ is selected from

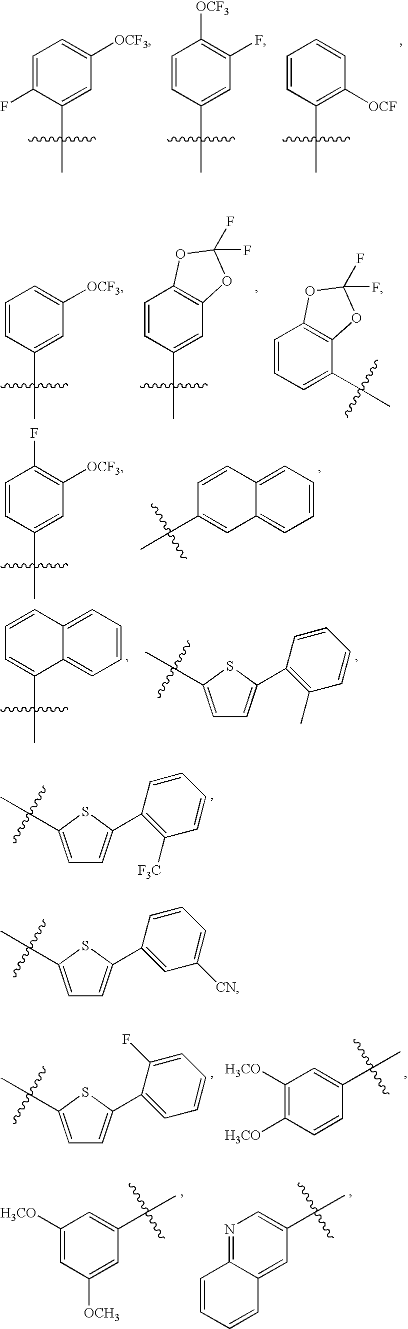

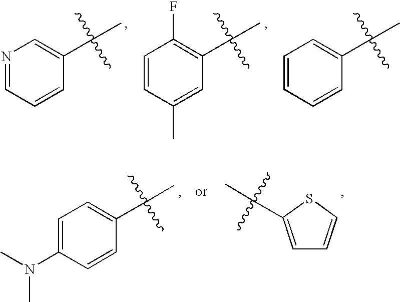

wherein the selection is consistent with the corresponding definition of R₅ or R₁₅ as recited above.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

| COMPOUND NO | [M + H]+ | STRUCTURE | | |
|---|---|---|---|---|
| 1 | 389.1 | 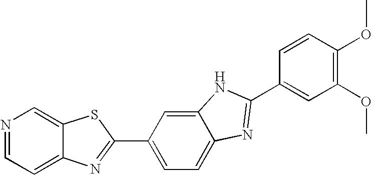 | A | B |
| 2 | 389.1 | 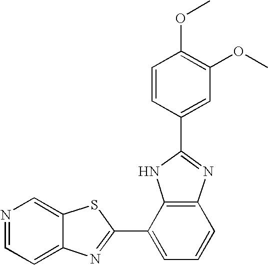 | A′ | A |
| 3 | 389.1 | 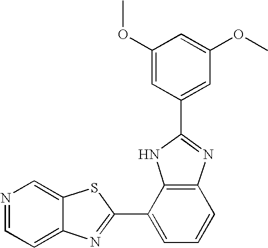 | A′ | B |
| 4 | 359.0 | 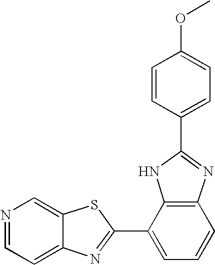 | A | A |
| 5 | 371.2 | 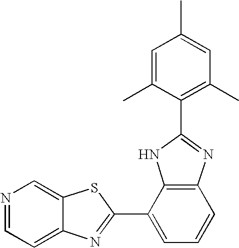 | A′ | B |
| 6 | 330.2 | 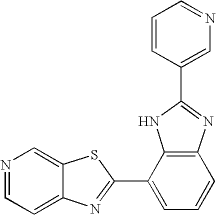 | A′ | B |
| 7 | 345.1 | 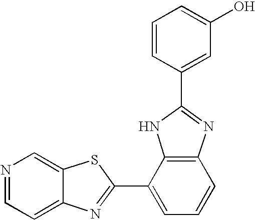 | A | A |
| 8 | 380.2 | 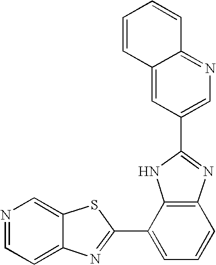 | A′ | A |
| 9 | 363.0 | 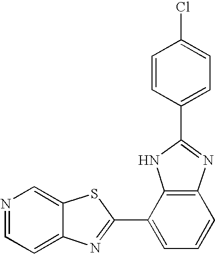 | NA | |
| 10 | 361.2 | 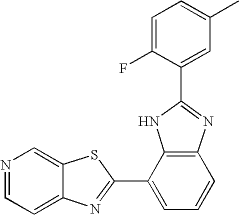 | A′ | B |
| 11 | 332.4 | 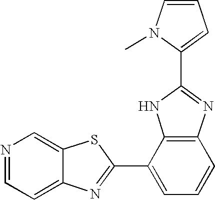 | A | A |
| 12 | 329.2 | 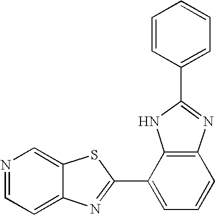 | A′ | A |
| 13 | 372.3 | 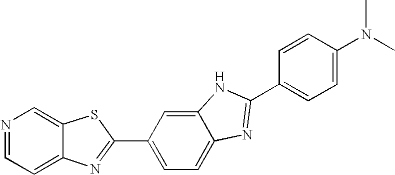 | A′ | B |
| 19 | 330.2 | 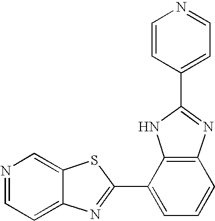 | A | B |
| 20 | 335.2 | 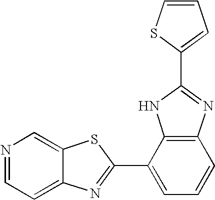 | A′ | A |
| 21 | 395.2 | 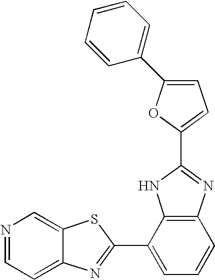 | NA | |
| 22 | 372.9 | 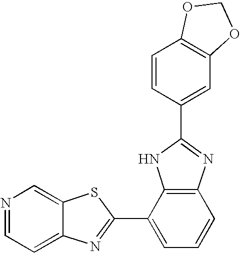 | A | B |
| 23 | 373.2 | 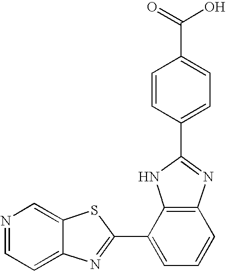 | A | B |
| 24 | 395.1 | 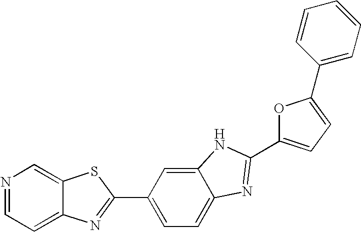 | NA | |
| 25 | 414.3 | 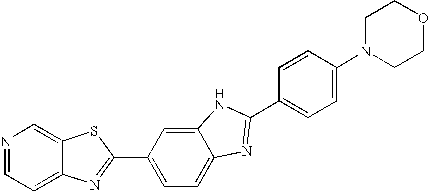 | A′ | B |
| 26 | 333.1 | 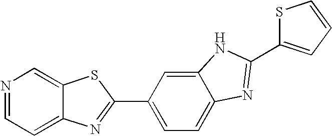 | A′ | B |
| 27 | 372.9 | 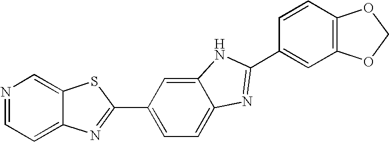 | A′ | B |
| 28 | 361.0 | 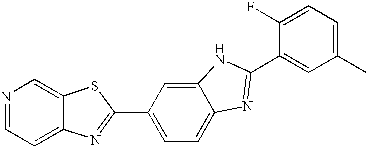 | | |
| 29 | 330.0 | 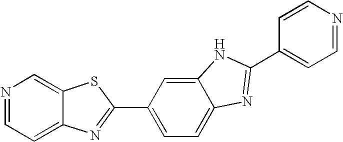 | A′ | B |
| 30 | 405.3 | 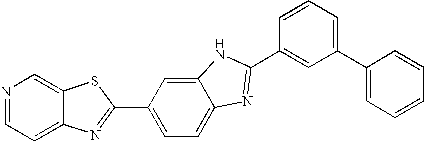 | NA | |
| 31 | 333.0 | 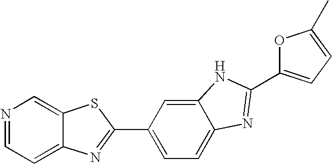 | A′ | A |
| 32 | | 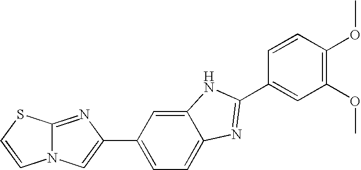 | | |
| 33 | | 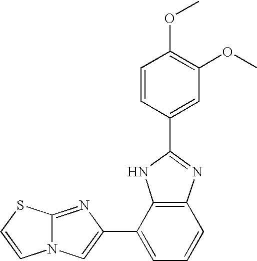 | | |
| 34 | | 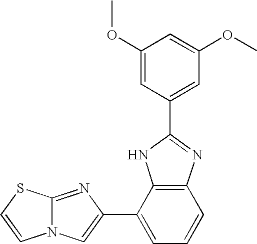 | | |
| 35 | | 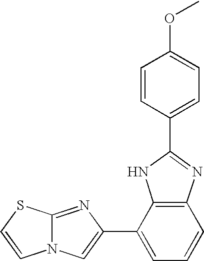 | | |
| 36 | | 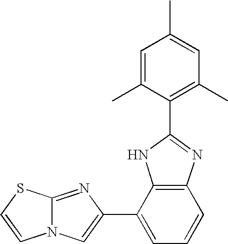 | | |
| 37 | | 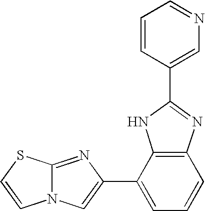 | | |
| 38 | | 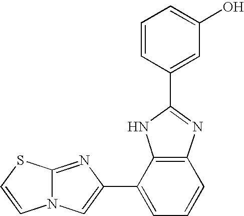 | | |
| 39 | | 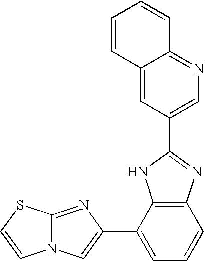 | | |
| 40 | | 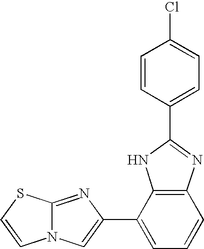 | | |
| 41 | | 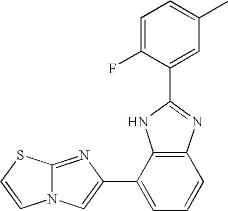 | | |
| 42 | | 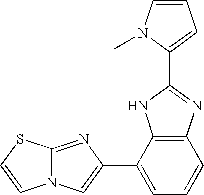 | | |
| 43 | | 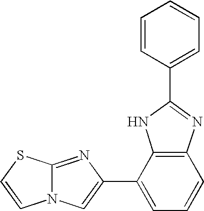 | | |
| 44 | | 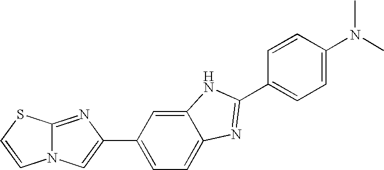 | | |
| 45 | | 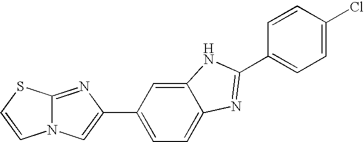 | | |
| 46 | | 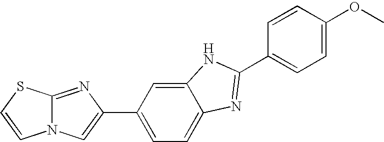 | | |
| 47 | | 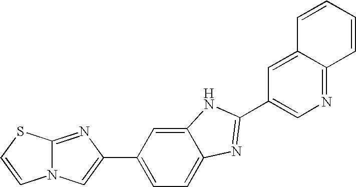 | | |
| 48 | | 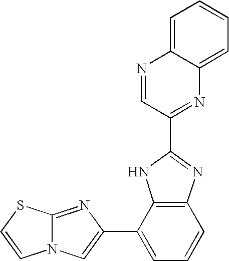 | | |
| 49 | | 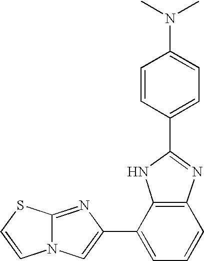 | | |
| 50 | | 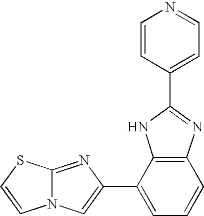 | | |
| 51 | | 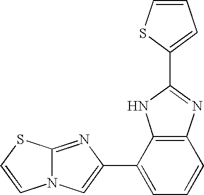 | | |
| 52 | | 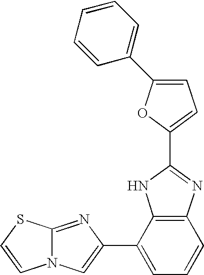 | | |
| 53 | | 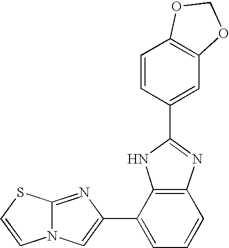 | | |
| 54 | | 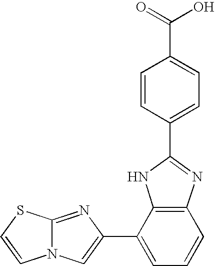 | | |
| 55 | | 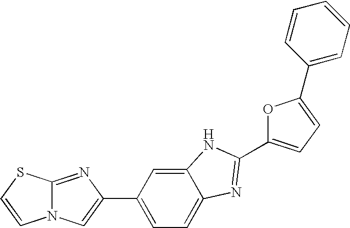 | | |
| 56 | | 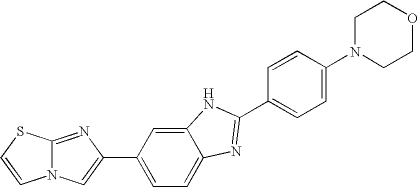 | | |
| 57 | | 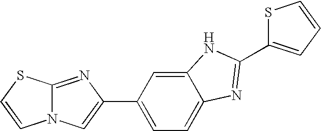 | | |
| 58 | | 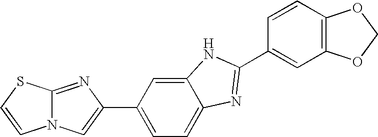 | | |
| 59 | | 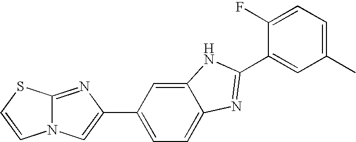 | | |
| 60 | | 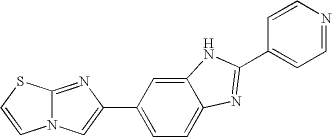 | | |
| 61 | | 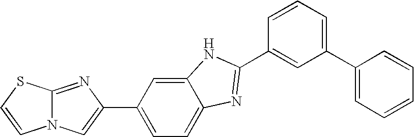 | | |
| 62 | | 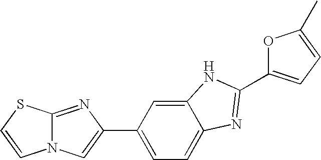 | | |

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C₁-C4 straight chained or branched alkyl group is also referred to as a “lower alkyl” group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group. Typically, a straight chained or branched alkenyl group has from 2 to about 20 carbon atoms, preferably from 2 to about 10, and a cyclic alkenyl group has from 3 to about 10 carbon atoms, preferably from 3 to about 8.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group. Typically, a straight chained or branched alkynyl group has from 2 to about 20 carbon atoms, preferably from 2 to about 10, and a cyclic alkynyl group has from 3 to about 10 carbon atoms, preferably from 3 to about 8.

A ring (e.g., 5- to 7-membered ring) or cyclic group includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

Aromatic (aryl) groups include carbocyclic aromatic groups such as phenyl, naphthyl, and antliracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuryl, tetraliyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (−Br, —Cl, —I and —F), —OR<a>, —O—COR<a>, —COR<a>, —C(O)R<3>, —CN, —NO<2>, —COOH, —COOR<a>, —OCO2R<3>, —C(O)NR<a>R<b>, —OC(O)NR<a>R<b>, —SO3H, —NH2, —NHR<a>, —N(R<a>R<b>), —COOR<3>, —CHO, —CONH2, —CONHR<3>, —CON(R<a>R<b>), —NHCOR<a>, —NRCOR<3>, —NHCONH2, —NHCONR<3>H, —NHC0N(R<a>R<b>), —NR<0>CONH2, —NR<0>CONR<3>H, —NR<c>CON(R<a>R<b>), —C(═NH)—NH2, —C(═NH)—NHR<3>, —C(═NH)—N(R<3>R<b>), —C(═NR[deg.])-NH2, —C(═NR<c>)—NHR<a>, —C(═NR<C>)—N(R<3>R<b>), —NH—C(═NH)—NH2, —NH—C(═NH)—NHR<3>, —NH—C(═NH)—N(R<a>R<b>), —NH—C(═NR[deg.])-NH2, —NH—C(═NR[deg.])-NHR<a>, —NH—C(═NR[deg.])-N(R<a>R<b>), —NR<d>H—C(═NH)—NH2, —NR<d>—C(═NH)—NHR<3>, —NR<d>—C(═NH)—N(R<a>R<b>), —NR<d>—C(═NR[deg.])-NH2, —NR<d>—C(═NR[deg.])-NHR<3>, —NR<d>—C(═NR[deg.])-N(R<3>R<b>), —NHNH2, —NHNHR<3>, —NHR<a>R<b>, —SO2NH2, —SO2NHR2, —SO2NR<a>R<b>, —CH═CHR<3>, —CH═CR<a>R<b>, —CR[deg.]=CR<a>R<b>, CR[deg.]=CHR<3>, —CR[deg.]=CR<3>R<b>, —CCR<3>, —SH, —SOkR<a>(k is 0, 1 or 2), —S(O)ₖOR<a>(k is 0, 1 or 2) and —NH—C(═NH)—NH2. R<3>—R<d> are each independently an aliphatic, substituted aliphatic, benzyl, substituted benzyl, aromatic or substituted aromatic group, preferably an alkyl, benzylic or aryl group. In addition, —NR<a>R<b>, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

A hydrogen-bond donating group is a functional group having a partially positively-charged hydrogen atom (e.g., —OH, —NH₂, —SH) or a group (e.g., an ester) that metabolizes into a group capable of donating a hydrogen bond.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH2)y—R<a>, N—(CH2)y—C(O)R<a>, N—(CH2)y—C(O)OR<a>, N—(CH2)y—S(O)2R<a>-, N—(CH2)y —S(O)2OR<a>, N—(CH2)y—C(O)NR<a>R<a>, etc., wherein R<a> is selected from hydrogen, lower alkyl, lower cycloalkyl, (C6-C14) aryl, phenyl, naphthyl, (C7-C20) arylalkyl and benzyl, wherein R<a> is optionally substituted; and y is an integer ranging from 0 to 6), optionally substituted heterocyclic groups (e.g., —(CH2)n—R<b>, —(CH2)n—C(0)-R<b>, —(CH2)n—O—(CH2)n—R<b>, wherein R<b> is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from O to 2). It should be understood that substituents present on R<a> or R<b> need not improve or increase water solubility over their unsubstituted counterparts to be within the scope of this definition. All that is required is that such substituents do not significantly reverse the improvement in water-solubility afforded by the unsubstituted R<a> or R<b> moiety.

In one embodiment, the solubilizing group increases the water-solubility of the corresponding compound lacking the solubilizing group at least 5-fold, preferably at least 10-fold, more preferably at least 20-fold and most preferably at least 50-fold. Exemplary soulbilizing groups include those disclosed in WO2007/019346, the contents of which is incorporated by reference herein.

Double bonds indicated in a structure as: ̂̂ are intended to include both the (E)- and (Z)-configuration. Preferably, double bonds are in the (E)-configuration.

A sugar is an aldehyde or ketone derivative of a straight-chain polyhydroxy alcohol, which contains at least three carbon atoms. A sugar can exist as a linear molecule or, preferably, as a cyclic molecule (e.g., in the pyranose or furanose form). Preferably, a sugar is a monosaccharide such as glucose or glucuronic acid. In embodiments of the invention where, for example, prolonged residence of a compound derivatized with a sugar is desired, the sugar is preferably a non-naturally occurring sugar. For example, one or more hydroxyl groups are substituted with another group, such as a halogen (e.g., chlorine). The stereochemical configuration at one or more carbon atoms can also be altered, as compared to a naturally occurring sugar. One example of a suitable non-naturally occurring sugar is sucralose.

A fatty acid is a carboxylic acid having a long-chained hydrocarbon moiety. Typically, a fatty acid has an even number of carbon atoms ranging from 12 to 24, often from 14 to 20. Fatty acids can be saturated or unsaturated and substituted or unsubstituted, but are typically unsubstituted. Fatty acids can be naturally or non-naturally occurring. In embodiments of the invention where, for example, prolonged residence time of a compound having a fatty acid moiety is desired, the fatty acid is preferably non-naturally occurring. The acyl group of a fatty acid consists of the hydrocarbon moiety and the carbonyl moiety of the carboxylic acid functionality, but excludes the —OH moiety associated with the carboxylic acid functionality.

Also included in the present invention are salts, particularly pharmaceutically acceptable salts, of the sirtuin-modulating compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterfoil (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monoliydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, niethoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Exemplary Uses

In certain aspects, the invention provides methods for modulating the level and/or activity of a HAT and methods of use thereof.

In certain embodiments, the invention provides methods for using HAT-modulating compounds wherein the HAT-modulating compounds inhibit a HAT, e.g., decrease the level and/or activity of a HAT. HAT-modulating compounds that decrease the level and/or activity of a HAT may be useful for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. The methods comprise administering to a subject in need thereof a pharmaceutically effective amount of a HAT-modulating compound, e.g., a HAT-inhibiting compound.

In other embodiments, the invention provides methods for using HAT-modulating compounds wherein the HAT-modulating compounds increase HAT activity, e.g., increase the level and/or activity of a HAT. HAT-modulating compounds that increase the level and/or activity of a HAT may be useful for a variety of therapeutic application including, for example, increasing cellular sensitivity to stress (including increasing radiosensitivity and/or chemosensitivity), increasing the amount and/or rate of apoptosis, treatment of cancer (optionally in combination another chemotherapeutic agent), stimulation of appetite, and/or stimulation of weight gain, etc. The methods comprise administering to a subject in need thereof a pharmaceutically effective amount of a HAT-modulating compound, e.g., a HAT-activating compound.

In certain embodiments, the HAT-modulating compounds described herein may be taken alone or in combination with other compounds. In one embodiment, a mixture of two or more HAT-modulating compounds may be administered to a subject in need thereof. In another embodiment, a HAT-modulating compound that decreases the level and/or activity of a HAT may be administered with one or more of the following compounds: resveratrol, butein, fisetin, piceatannol, or quercetin. In an exemplary embodiment, a HAT-modulating compound that decreases the level and/or activity of a HAT may be administered in combination with nicotinic acid. In another embodiment, a HAT-modulating compound that increases the level and/or activity of a HAT may be administered with one or more of the following compounds: nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4<T>, 5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (S′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone), sirtinol; and splitomicin (see e.g., Howitz et al. (2003) Nature 425:191; Grozinger et al. (2001) J. Biol. Chem. 276:38837; Dedalov et al. (2001) PAULS'98:15113; and Hirao et al. (2003) J. Biol. Chem 278:52773).

In yet another embodiment, one or more HAT-modulating compounds may be administered with one or more therapeutic agents for the treatment or prevention of various diseases, including, for example, cancer, diabetes, neurodegenerative diseases, cardiovascular disease, blood clotting; inflammation, flushing, obesity, ageing, stress, etc. In various embodiments, combination therapies comprising a HAT-modulating compound may refer to (1) pharmaceutical compositions that comprise one or more HAT-modulating compounds in combination with one or more therapeutic agents (e.g., one or more therapeutic agents described herein); and (2) co-administration of one or more HAT-modulating compounds with one or more therapeutic agents wherein the HAT-modulating compound and therapeutic agent have not been formulated in the same compositions (but may be present within the same kit or package, such as a blister pack or other multi-chamber package; connected, separately sealed containers (e.g., foil pouches) that can be separated by the user; or a kit where the HAT modulating compound(s) and other therapeutic agent(s) are in separate vessels). When using separate formulations, the HAT-modulating compound may be administered at the same, intermittent, staggered, prior to, subsequent to, or combinations thereof, with the administration of another therapeutic agent.

### Aging/Stress

In one embodiment, the invention provides a method extending the lifespan of a cell, extending the proliferative capacity of a cell, slowing ageing of a cell, promoting the survival of a cell, delaying cellular senescence in a cell, mimicking the effects of calorie restriction, increasing the resistance of a cell to stress, or preventing apoptosis of a cell, by contacting the cell with a HAT-modulating compound of the invention that decreases the level and/or activity of a HAT. In an exemplary embodiment, the methods comprise contacting the cell with a HAT-inhibiting compound. The methods described herein may be used to increase the amount of time that cells, particularly primary cells (i.e., cells obtained from an organism, e.g., a human), may be kept alive in a cell culture. Embryonic stem (ES) cells and pluripotent cells, and cells differentiated therefrom, may also be treated with a HAT-modulating compound that decreases the level and/or activity of a HAT protein to keep the cells, or progeny thereof, in culture for longer periods of time. Such cells can also be used for transplantation into a subject, e.g., after ex vivo modification.

In one embodiment, cells that are intended to be preserved for long periods of time may be treated with a HAT-modulating compound that decreases the level and/or activity of a HAT. The cells may be in suspension (e.g., blood cells, serum, biological growth media, etc. or in tissues or organs. For example, blood collected from an individual for purposes of transfusion may be treated with a HAT-modulating compound that decreases the level and/or activity of a HAT to preserve the blood cells for longer periods of time. Additionally, blood to be used for forensic purposes may also be preserved using a HAT-modulating compound that decreases the level and/or activity of a HAT. Other cells that may be treated to extend their lifespan or protect against apoptosis include cells for consumption, e.g., cells from non-human mammals (such as meat) or plant cells (such as vegetables). HAT-modulating compounds that decrease the level and/or activity of a HAT may also be applied during developmental and growth phases in mammals, plants, insects or microorganisms, in order to, e.g., alter, retard or accelerate the developmental and/or growth process.

In another embodiment, HAT-modulating compounds that decrease the level and/or activity of a HAT protein may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the HAT-modulating compound prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a HAT-modulating compound or may have a subset of cells/tissue treated locally with a HAT-modulating compound that decreases the level and/or activity of a HAT. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc. In yet other embodiments, cells may be treated with a HAT-modulating compound that decreases the level and/or activity of a HAT in vivo, e.g., to increase their lifespan or prevent apoptosis. For example, skin can be protected from aging (e.g., developing wrinkles, loss of elasticity, etc.) by treating skin or epithelial cells with a HAT-modulating compound that decreases the level and/or activity of a HAT. In an exemplary embodiment, skin is contacted with a pharmaceutical or cosmetic composition comprising a HAT-modulating compound that decreases the level and/or activity of a HAT. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, HAT-modulating compounds that decrease the level and/or activity of a HAT maybe used for the treatment of wounds and/or burns to promote healing, including, for example, first-, second- or third-degree burns and/or a thermal, chemical or electrical burns. The formulations may be administered topically, to the skin or mucosal tissue, as an ointment, lotion, cream; microemulsion, gel, solution or the like, as further described herein, within the context of a dosing regimen effective to bring about the desired result.

Topical formulations comprising one or more HAT-modulating compounds that decrease the level and/or activity of a HAT may also be used as preventive, e.g., chemopreventive, compositions. When used in a chemopreventive method, susceptible skin is treated prior to any visible condition in a particular individual.

HAT-modulating compounds may be delivered locally or systemically to a subject. In one embodiment, a HAT-modulating compound is delivered locally to a tissue or organ of a subject by injection, topical formulation, etc.

In another embodiment, a HAT-modulating compound that decreases the level and/or activity of a HAT may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a HAT-modulating compound that decreases the level and/or activity of a HAT may be administered to a subject in order to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan.

HAT-modulating compounds that decrease the level and/or activity of a HAT may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. HAT-modulating compounds that decrease the level and/or activity of a HAT can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease; retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

HAT-modulating compounds that decrease the level and/or activity of a HAT can also be administered to a subject suffering from an acute disease, e.g., damage to an organ or tissue, e.g., a subject suffering from stroke or myocardial infarction or a subject suffering from a spinal cord injury. HAT-modulating compounds that decrease the level and/or activity of a HAT may also be used to repair an alcoholic's liver.

Cardiovascular Disease

In another embodiment, the invention provides a method for treating and/or preventing a cardiovascular disease by administering to a subject in need thereof a HAT-modulating compound that decreases the level and/or activity of a HAT.

Cardiovascular diseases that can be treated or prevented using the HAT-modulating compounds that decrease the level and/or activity of a HAT include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The HAT-modulating compounds that decrease the level and/or activity of a HAT may also be used for increasing HDL levels in plasma of an individual.

Yet other disorders that may be treated with HAT-modulating compounds that decrease the level and/or activity of a HAT include restenosis, e.g., following coronary intervention, and disorders relating to an abnormal level of high density and low density cholesterol.

In one embodiment, a HAT-modulating compound that decreases the level and/or activity of a HAT may be administered as part of a combination therapeutic with another cardiovascular agent including, for example, an antiarrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

In one embodiment, a HAT-modulating compound that decreases the level and/or activity of a HAT may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocamide, procainamide, encamide, flecanide, tocamide, phenyloin, propafenone, quinidine, disopyramide, and flecamide. Type II antiarrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltaizem, digitalis, adenosine, nickel chloride, and magnesium ions.

In another embodiment, a HAT-modulating compound that decreases the level and/or activity of a HAT may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; anti-arrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorthalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetanide, triamterene, amiloride and spironolactone and salts of such 5 compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex (APSAC, Eminase, Beecham Laboratories), or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this invention within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin 12), an endothelin receptor blocking drug (such as bosentan), diltiazem, nicorandil, and nitroglycerin. Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na<4>VCa<2+> channel inhibitory drugs, and K<+> channel opening drugs. Examples of the brain metabolic stimulants include amantadine, tiapride, and gamma-aminobutyric acid. Examples of the anticoagulant include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene. Examples of the thrombolytic drug include urokinase, tissue-type plasminogen activators (such as alteplase, tisol[alpha]nase, nateplase, pamiteplase, monteplase, and rateplase), and nasaruplase.

Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, d[upsilon]tiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), [beta]-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), [alpha]-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine.

Examples of the antianginal drug include nitrate drugs (such as amyl nitrite, nitroglycerin, and isosorbide), [beta]-adrenaline receptor blocking drags (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calcium channel blocking drags (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenyl amine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline) trimetazidine, dipyridamole, etafenone, dilazep, trapidil, nicorandil, enoxaparin, and aspirin.

Examples of the diuretic include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K<+> sparing diuretics (spironolactone, triamterene, andpotassiumcanrenoate), osmotic diuretics (such as isosorbide, D-mannitol, and glycerin), nonthiazide diuretics (such as meticrane, tripamide, chlorthalidone, and mefruside), and acetazolamide. Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as aminone, olprinone, and milrinone), denopamine, ubidecarenone, pimobendan, levosimendan, aminoethylsulfonic acid, vesnarinone, carperitide, and colforsin daropate. Examples of the antiarrhythmic drug include ajmaline, pirmenol, procainamide, cibenzoline, disopyramide, quinidine, aprindine, mexiletine, lidocaine, phenyloin, pilsicamide, propafenone, flecamide, atenolol, acebutolol, sotalol, propranolol, metoprolol, pindolol, amiodarone, nifekalant, diltiazem, bepridil, and verapamil. Examples of the antihyperlipidemic drag include atorvastatin, simvastatin, pravastatin sodium, fluvastatin sodium, clinofibrate, clof[iota]brate, simfibrate, fenof[iota]brate, bezafibrate, colestimide, and colestyramine. Examples of the immunosuppressant include azathioprine, mizoribine, cyclosporine, tacrolimus, gusperimus, and methotrexate.

### Cell Death/Cancer

HAT-modulating compounds that decrease the level and/or activity of a HAT may be administered to subjects who have recently received or are likely to receive a dose of radiation or toxin. In one embodiment, the dose of radiation: or toxin is received as part of a work-related or medical procedure, e.g., working in a nuclear power plant, flying an airplane, an X-ray, CAT scan, or the administration of a radioactive dye for medical imaging; in such an embodiment, the compound is administered as a prophylactic measure. In another embodiment, the radiation or toxin exposure is received unintentionally, e.g., as a result of an industrial accident, habitation in a location of natural radiation, terrorist act, or act of war involving radioactive or toxic material. In such a case, the compound is preferably administered as soon as possible after the exposure to inhibit apoptosis and the subsequent development of acute radiation syndrome.

HAT-modulating compounds may also be used for treating and/or preventing cancer. In certain embodiments, HAT-modulating compounds that decrease the level and/or activity of a HAT may be used for treating and/or preventing cancer. A decrease in the level and/or activity of a HAT may be useful for treating and/or preventing the incidence of age-related disorders, such as, for example, cancer. In other embodiments, HAT-modulating compounds that decrease the level and/or activity of a HAT may be used for treating or preventing cancer. Exemplary cancers that may be treated using a HAT-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Benign cell growth can also be treated, e.g., warts. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-I associated malignant and benign disorders can also be treated by administration of HAT-modulating compound. Alternatively, cells can be obtained from a subject, treated ex vivo to remove certain undesirable cells, e.g., cancer cells, and administered back to the same or a different subject.

Chemotherapeutic agents that may be coadministered with modulating compounds described herein as having anti-cancer activity (e.g., compounds that induce apoptosis, compounds that reduce lifespan or compounds that render cells sensitive to stress) include: aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disrupters such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP 16)); antibiotics such as dactinomycin (actinomycin D), daunorubicin, doxorabicin (adriamycin), idarubicin, antliracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin); procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorabicin (adriamycin), amsacrine, camptothecin, daunorabicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These chemotherapeutic agents may be used by themselves with a HAT-modulating compound described herein as inducing cell death or reducing lifespan or increasing sensitivity to stress and/or in combination with other chemotherapeutics agents.

In addition to conventional chemotherapeutics, the HAT-modulating compounds described herein as capable of inducing cell death or reducing lifespan can also be used with antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

Combination therapies comprising HAT-modulating compounds and a conventional chemotherapeutic agent may be advantageous over combination therapies known in the art because the combination allows the conventional chemotherapeutic agent to exert greater effect at lower dosage. In a preferred embodiment, the effective dose (ED₅₀) for a chemotherapeutic agent, or combination of conventional chemotherapeutic agents, when used in combination with a HAT-modulating compound is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a HAT-modulating compound described herein can be at least 2 fold greater than the TI for conventional chemotherapeutic regimen alone, and even more preferably at 5 fold, 10 fold or even 25 fold greater.

Neuronal Diseases/Disorders

In certain aspects, HAT-modulating compounds that decrease the level and/or activity of a HAT can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which s [alpha]B [kappa]30 B+-[identical to] may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. HAT-modulating compounds that decrease the level and/or activity of a decreases can be used to treat these disorders and others as described below. AD is a chronic, incurable, and unstoppable CNS disorder that occurs gradually, resulting in memory loss, unusual behavior, personality changes, and a decline in thinking abilities. These losses are related to the death of specific types of brain cells and the breakdown of connections and their supporting network (e.g. glial cells) between them. AD has been described as childhood development in reverse. In most people with AD, symptoms appear after the age 60. The earliest symptoms include loss of recent memory, faulty judgment, and changes in personality. Later in the disease, those with AD may forget how to do simple tasks like washing their hands. Eventually people with AD lose all reasoning abilities and become dependent on other people for their everyday care. Finally, the disease becomes so debilitating that patients are bedridden and typically develop coexisting illnesses.

PD is a chronic, incurable, and unstoppable CNS disorder that occurs gradually and results in uncontrolled body movements, rigidity, tremor, and dyskinesia. These motor system problems are related to the death of brain cells in an area of the brain that produces dopamine, a chemical that helps control muscle activity. In most people with PD, symptoms appear after age 50. The initial symptoms of PD are a pronounced tremor affecting the extremities, notably in the hands or lips. Subsequent characteristic symptoms of PD are stiffness or slowness of movement, a shuffling walk, stooped posture, and impaired balance. There are wide ranging secondary symptoms such as memory loss, dementia, depression, emotional changes, swallowing difficulties, abnormal speech, sexual dysfunction, and bladder and bowel problems. These symptoms will begin to interfere with routine activities, such as holding a fork or reading a newspaper. Finally, people with PD become so profoundly disabled that they are bedridden. ALS (motor neuron disease) is a chronic, incurable, and unstoppable CNS disorder that attacks the motor neurons, components of the CNS that connect the brain to the skeletal muscles. In ALS, the motor neurons deteriorate and eventually die, and though a person's brain normally remains fully functioning and alert, the command to move never reaches the muscles. Most people who get ALS are between 40 and 70 years old. The first motor neurons that weaken are those controlling the arms or legs. Those with ALS may have trouble walking, they may drop things, fall, slur their speech, and laugh or cry uncontrollably. Eventually the muscles in the limbs begin to atrophy from disuse. This muscle weakness will become debilitating and a person will need a wheel chair or become unable to function out of bed. The causes of these neurological diseases have remained largely unknown.

They are conventionally defined as distinct diseases, yet clearly show extraordinary similarities in basic processes and commonly demonstrate overlapping symptoms far greater than would be expected by chance alone. Current disease definitions fail to properly deal with the issue of overlap and a new classification of the neurodegenerative disorders has been called for.

HD is another neurodegenerative disease resulting from genetically programmed degeneration of neurons in certain areas of the brain. This degeneration causes uncontrolled movements, loss of intellectual faculties, and emotional disturbance. HD is a familial disease, passed from parent to child through a dominant mutation in the wild-type gene. Some early symptoms of HD are mood swings, depression, irritability or trouble driving, learning new things, remembering a fact, or making a decision. As the disease progresses, concentration on intellectual tasks becomes increasingly difficult and the patient may have difficulty feeding himself or herself and swallowing. Tay-Sachs disease and Sandhoff disease are glycolipid storage diseases caused by the lack of lysosomal [beta]-hexosaminidase (Gravel et al., in The Metabolic Basis of Inherited Disease, eds. Scriver et al., McGraw-Hill, New York, pp. 2839-2879, 1995). In both disorders, GM2 ganglioside and related glycolipidssubstrates for [beta]-hexosaminidase accumulate in the nervous system and trigger acute neurodegeneration.

In the most severe forms, the onset of symptoms begins in early infancy. A precipitous neurodegenerative course then ensues, with affected infants exhibiting motor dysfunction, seizure, visual loss, and deafness. Death usually occurs by 2-5 years of 5 age. Neuronal loss through an apoptotic mechanism has been demonstrated (Huang et al., Hum. MoI. Genet. 6: 1879-1885, 1997).

It is well-known that apoptosis plays a role in AIDS pathogenesis in the immune system. However, HIV-I also induces neurological disease. Shi et al. (J. Clin. Invest. 98: 1979-1990, 1996) examined apoptosis induced by HIV-I infection of the CNS in an in vitro model and in brain tissue from AIDS patients, and found that HIV-I infection of primary brain cultures induced apoptosis in neurons and astrocytes in vitro. Apoptosis of neurons and astrocytes was also detected in brain tissue from 10/11 AIDS patients, including 5/5 patients with HIV-I dementia and 4/5 nondemented patients.

There are four main peripheral neuropathies associated with HIV, namely sensory neuropathy, AIDP/CIPD, drug-induced neuropathy and CMV-related.

The most common type of neuropathy associated with AIDS is distal symmetrical polyneuropathy (DSPN). This syndrome is a result of nerve degeneration and is characterized by numbness and a sensation of pins and needles. DSPN causes few serious abnormalities and mostly results in numbness or tingling of the feet and slowed reflexes at the ankles. It generally occurs with more severe immunosuppression and is steadily progressive. Treatment with tricyclic antidepressants relieves symptoms but does not affect the underlying nerve damage.

A less frequent, but more severe type of neuropathy is known as acute or chronic inflammatory demyelinating polyneuropathy (AIDP/CIDP). In AIDP/CIDP there is damage to the fatty membrane covering the nerve impulses. This kind of neuropathy involves inflammation and resembles the muscle deterioration often identified with long-term use of AZT. It can be the first manifestation of HIV infection, where the patient may not complain of pain, but fails to respond to standard reflex tests. This kind of neuropathy may be associated with seroconversion, in which case it can sometimes resolve spontaneously. It can serve as a sign of HIV infection and indicate that it might be time to consider antiviral therapy. AIDP/CIDP may be auto-immune in origin.

Drug-induced, or toxic, neuropathies can be very painful. Antiviral drugs commonly cause peripheral neuropathy, as do other drugs e.g. vincristine, dilantin (an anti-seizure medication), high-dose vitamins, isoniazid, and folic acid antagonists. Peripheral neuropathy is often used in clinical trials for antivirals as a dose-limiting side effect, which means that more drugs should not be administered. Additionally, the use of such drugs can exacerbate otherwise minor neuropathies. Usually, these drug-induced neuropathies are reversible with the discontinuation of the drug.

CMV causes several neurological syndromes in AIDS, including encephalitis, myelitis, and polyradiculopathy.

Neuronal loss is also a salient feature of prion diseases, such as Creutzfeldt-Jakob disease in human, BSE in cattle (mad cow disease), Scrapie Disease in sheep andmgoats, and feline spongiform encephalopathy (FSE) in cats. HAT-modulating compounds that decrease the level and/or activity of a HAT may be useful for treating or preventing neuronal loss due to these prior diseases.

In another embodiment, a HAT-modulating compound that decreases the level and/or activity of a HAT may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy. The most distal portions of axons are usually the first to degenerate, and axonal atrophy advances slowly towards the nerve's cell body. If the noxious stimulus is removed, regeneration is possible, though prognosis decreases depending on the duration and severity of the stimulus. Those with distal axonopathies usually present with symmetrical glove-stocking sensori-motor disturbances. Deep tendon reflexes and autonomic nervous system (ANS) functions are also lost or diminished in affected areas.

Diabetic neuropathies are neuropathic disorders that are associated with diabetes mellitus. These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy. Clinical manifestations of diabetic neuropathy include, for example, sensorimotor polyneuropathy such as numbness, sensory loss, dysesthesia and nighttime pain; autonomic neuropathy such as delayed gastric emptying or gastroparesis; and cranial neuropathy such as oculomotor (3rd) neuropathies or Mononeuropathies of the thoracic or lumbar spinal nerves. Peripheral neuropathy is the medical term for damage to nerves of the peripheral nervous system, which may be caused either by diseases of the nerve or from the side-effects of systemic illness. Peripheral neuropathies vary in their presentation and origin, and may affect the nerve or the neuromuscular junction. Major causes of peripheral neuropathy include seizures, nutritional deficiencies, and HIV, though diabetes is the most likely cause. Mechanical pressure from staying in one position for too long, a tumor, intraneural hemorrhage, exposing the body to extreme conditions such as radiation, cold temperatures, or toxic substances can also cause peripheral neuropathy.

In an exemplary embodiment, a HAT-modulating compound that decreases the level and/or activity of a HAT may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

MS is a chronic, often disabling disease of the central nervous system. Various and converging lines of evidence point to the possibility that the disease is caused by a disturbance in the immune function, although the cause of this disturbance has not been established. This disturbance permits cells of the immune system to “attack” myelin, the fat containing insulating sheath that surrounds the nerve axons located in the central nervous system (“CNS”). When myelin is damaged, electrical pulses cannot travel quickly or normally along nerve fiber pathways in the brain and spinal cord. This results in disruption of normal electrical conductivity within the axons, fatigue and disturbances of vision, strength, coordination, balance, sensation, and bladder and bowel function.

As such, MS is now a common and well-known neurological disorder that is characterized by episodic patches of inflammation and demyelination which can occur anywhere in the CNS. However, almost always without any involvement of the peripheral nerves associated therewith. Demyelination produces a situation analogous to that resulting from cracks or tears in an insulator surrounding an electrical cord. That is, when the insulating sheath is disrupted, the circuit is “short circuited” and the electrical apparatus associated therewith will function intermittently or nor at all. Such loss of myelin surrounding nerve fibers results in short circuits in nerves traversing the brain and the spinal cord that thereby result in symptoms of MS. It is further found that such demyelination occurs in patches, as opposed to along the entire CNS. In addition, such demyelination may be intermittent. Therefore, such plaques are disseminated in both time and space.

It is believed that the pathogenesis involves a local disruption of the blood brain barrier which causes a localized immune and inflammatory response, with consequent damage to myelin and hence to neurons. Clinically, MS exists in both sexes and can occur at any age. However, its most common presentation is in the relatively young adult, often with a single focal lesion such as a damage of the optic nerve, an area of anesthesia (loss of sensation), or paraesthesia (localize loss of feeling), or muscular weakness. In addition, vertigo, double vision, localized pain, incontinence, and pain in the arms and legs may occur upon flexing of the neck, as well as a large variety of less common symptoms.

An initial attack of MS is often transient, and it may be weeks, months, or years before a further attack occurs. Some individuals may enjoy a stable, relatively event free condition for a great number of years, while other less fortunate ones may experience a continual downhill course ending in complete paralysis. There is, most commonly, a series of remission and relapses, in which each relapse leaves a patient somewhat worse than before. Relapses may be triggered by stressful events, viral infections or toxins. Therein, elevated body temperature, i.e., a fever, will make the condition worse, or as a reduction of temperature by, for example, a cold bath, may make the condition better. In yet another embodiment, a HAT-modulating compound that decreases the level and/or activity of a HAT may be used to treat trauma to the nerves, including, trauma due to disease, injury (including surgical intervention), or environmental trauma (e.g., neurotoxins, alcoholism, etc.).

HAT-modulating compounds that decrease the level and/or activity of a HAT may also be useful to prevent, treat, and alleviate symptoms of various PNS disorders, such as the ones described below. The PNS is composed of the nerves that lead to or branch off from the spinal cord and CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

Peripheral neuropathy is a widespread disorder, and there are many underlying causes. Some of these causes are common, such as diabetes, and others are extremely rare, such as acrylamide poisoning and certain inherited disorders. The most common worldwide cause of peripheral neuropathy is leprosy. Leprosy is caused by the bacterium Mycobacterium leprae, which attacks the peripheral nerves of affected people.

Leprosy is extremely rare in the United States, where diabetes is the most commonly known cause of peripheral neuropathy. It has been estimated that more than 17 million people in the United States and Europe have diabetes-related polyneuropathy. Many neuropathies are idiopathic; no known cause can be found. The most common of the inherited peripheral neuropathies in the United States is Charcot-Marie-Tooth disease, which affects approximately 125,000 persons.

Another of the better known peripheral neuropathies is Guillain-Barre syndrome, which arises from complications associated with viral illnesses, such as cytomegalovirus, Epstein-Barr vims, and human immunodeficiency virus (HIV), or bacterial infection, including Campylobacter jejuni and Lyme disease. The worldwide incidence rate is approximately 1.7 cases per 100,000 people annually. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the PNS and the CNS, as well as other body tissues.

Other PNS diseases treatable with HAT-modulating compounds that decrease the level and/or activity of a HAT include: Brachial Plexus Neuropathies (diseases of the cervical and first thoracic roots, nerve trunks, cords, and peripheral nerve components of the brachial plexus. Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp. 1351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy (see Adams et al., Principles of Neurology, 6th ed, pl 325); mononeuropathies (disease or trauma involving a single peripheral nerve in isolation, or out of proportion to evidence of diffuse peripheral nerve dysfunction). Mononeuritis multiplex refers to a condition characterized by multiple isolated nerve injuries. Mononeuropathies may result from a wide variety of causes, including ischemia; traumatic injury; compression; connective tissue diseases; cumulative trauma disorders; and other conditions; Neuralgia (intense or aching pain that occurs along the course or distribution of a peripheral or cranial nerve); Peripheral Nervous System Neoplasms (neoplasms which arise from peripheral nerve tissue). This includes neurofibromas; Schwannomas; granular cell tumors; and malignant peripheral nerve sheath tumors (see DeVita Jr et al., Cancer: Principles and Practice of Oncology, 5th ed, ppl 750-1); and Nerve Compression Syndromes (mechanical compression of nerves or nerve roots from internal or external causes). These may result in a conduction block to nerve impulses, due to, for example, myelin sheath dysfunction, or axonal loss. The nerve and nerve sheath injuries may be caused by ischemia; inflammation; or a direct mechanical effect; Neuritis (a general term indicating inflammation of a peripheral or cranial nerve). Clinical manifestation may include pain; paresthesias; paresis; or hyperesthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

In another embodiment, a HAT inhibiting compound may be used to treat or prevent chemotherapeutic induced neuropathy. The HAT modulating compounds” may be administered prior to administration of the chemotherapeutic agent, concurrently with administration of the chemotherapeutic drug, and/or after initiation of administration of the chemotherapeutic drug. If the HAT inhibiting compound is administered after the initiation of administration of the chemotherapeutic drug, it is desirable that the HAT inhibiting compound be administered prior to, or at the first signs, of chemotherapeutic induced neuropathy.

Chemotherapy drugs can damage any part of the nervous system. Encephalopathy and myelopathy are fortunately very rare. Damage to peripheral nerves is much more common and can be a side effect of treatment experienced by people with cancers, such as lymphoma. Most of the neuropathy affects sensory rather than motor nerves. Thus, the common symptoms are tingling, numbness or a loss of balance. The longest nerves in the body seem to be most sensitive hence the fact that most patients will report numbness or pins and needles in their hands and feet.

The chemotherapy drugs which are most commonly associated with neuropathy, are the Vinca alkaloids (anti-cancer drugs originally derived from a member of the periwinkle—the Vinca plant genus) and a platinum-containing drug called Cisplatin. The Vinca alkaloids include the drugs vinblastine, vincristine and vindesine. Many combination chemotherapy treatments for lymphoma for example CHOP and CVP contain vincristine, which is the drug known to cause this problem most frequently. Indeed, it is the risk of neuropathy that limits the dose of vincristine that can be administered. Studies that have been performed have shown that most patients will lose some reflexes in their legs as a result of treatment with vincristine and many will experience some degree of tingling (paresthesia) in their fingers and toes. The neuropathy does not usually manifest itself right at the start of the treatment but generally comes on over a period of a few weeks. It is not essential to stop the drug at the first onset of symptoms, but if the neuropathy progresses this may be necessary. It is very important that patients should report such symptoms to their doctors, as the nerve damage is largely reversible if the drug is discontinued. Most doctors will often reduce the dose of vincristine or switch to another form of Vinca alkaloid such as vinblastine or vindesine if the symptoms are mild. Occasionally, the nerves supplying the bowel are affected causing abdominal pain and constipation.

In another embodiment, a HAT inhibiting compound may be used to treat or prevent a polyglutamine disease. Huntington's Disease (HD) and Spinocerebellar type 1 (SCA1) are just two examples of a class of genetic diseases caused by dynamic mutations involving the expansion of triplet sequence repeats. In reference to this common mechanism, these disorders are called trinucleotide repeat diseases. At least 14 such diseases are known to affect human beings. Nine of them, including SCA1 and Huntington's disease, have CAG as the repeated sequence. Since CAG codes for an amino acid called glutamine, these nine trinucleotide repeat disorders are collectively known as polyglutamine diseases.

Although the genes involved in different polyglutamine diseases have little in common, the disorders they cause follow a strikingly similar course. Each disease is characterized by a progressive degeneration of a distinct group of nerve cells. The major symptoms of these diseases are similar, although not identical, and usually affect people in midlife. Given the similarities in symptoms, the polyglutamine diseases are hypothesized to progress via common cellular mechanisms. In recent years, scientists have made great strides in unraveling what the mechanisms are.

Above a certain threshold, the greater the number of glutamine repeats in a protein, the earlier the onset of disease and the more severe the symptoms. This suggests that abnormally long glutamine tracts render their host protein toxic to nerve cells.

To test this hypothesis, scientists have generated genetically engineered mice expressing proteins with long polyglutamine tracts. Regardless of whether the mice express full-length proteins or only those portions of the proteins containing the polyglutamine tracts, they develop symptoms of polyglutamine diseases. This suggests that a long polyglutamine tract by itself is damaging to cells and does not have to be part of a functional protein to cause its damage.

For example, it is thought that the symptoms of SCA1 are not directly caused by the loss of normal ataxin-1 function but rather by the interaction between ataxin-1 and another protein called LANP. LANP is needed for nerve cells to communicate with one another and thus for their survival. When the mutant ataxin-1 protein accumulates inside nerve cells, it “traps” the LANP protein, interfering with its normal function.

Many transcription factors have also been found in neuronal inclusions in different diseases. It is possible that these transcription factors interact with the polyglutamine-containing proteins and then become trapped in the neuronal inclusions. This in turn might keep the transcription factors from turning genes on and off as needed by the cell. Another observation is hypoacetylation of histones in affected cells. This has led to the hypothesis that Class I/II Histone Deacetylase (HDAC I/II) inhibitors, which are known to increase histone acetylation, may be a novel therapy for polyglutamine diseases (U.S. patent application Ser. No. 10/476,627; “Method of treating neurodegenerative, psychiatric, and other disorders with deacetylase inhibitors”).

In yet another embodiment, the invention provides a method for treating or preventing neuropathy related to ischemic injuries or diseases, such as, for example, coronary heart disease (including congestive heart failure and myocardial infarctions), stroke, emphysema, hemorrhagic shock, peripheral vascular disease (upper and lower extremities) and transplant related injuries.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. By way of example, the normal amount of perfusion to brain gray matter in humans is about 60 to 70 mL/100 g of brain tissue/min. Death of central nervous system cells typically occurs when the flow of blood falls below approximately 8-10 mL/100 g of brain tissue/min, while at slightly higher levels (i.e. 20-35 mL/100 g of brain tissue/min) the tissue remains alive but not able to function. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a HAT inhibiting compound to a subject to treat a central nervous system ischemic condition. A number of central nervous system ischemic conditions may be treated by the HAT inhibiting compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. Generally speaking, brain stem strokes strike the brain stem, which control involuntary life-support functions such as breathing, blood pressure, and heartbeat. In another alternative of this embodiment, the stroke is a cerebellar stroke. Typically, cerebellar strokes impact the cerebellum area of the brain, which controls balance and coordination. In still another embodiment, the stroke is an embolic stroke. In general terms, embolic strokes may impact any region of the brain and typically result from the blockage of an artery by a vaso-occlusion. In yet another alternative, the stroke may be a hemorrhagic stroke. Like ischemic strokes, hemorrhagic stroke may impact any region of the brain, and typically result from a ruptured blood vessel characterized by a hemorrhage (bleeding) within or surrounding the brain. In a further embodiment, the stroke is a thrombotic stroke. Typically, thrombotic strokes result from the blockage of a blood vessel by accumulated deposits.

In another embodiment, the ischemic condition may result from a disorder that occurs in a part of the subject's body outside of the central nervous system, but yet still causes a reduction in blood flow to the central nervous system. These disorders may include, but are not limited to a peripheral vascular disorder, a venous thrombosis, a pulmonary embolus, arrhythmia (e.g. atrial fibrillation), a myocardial infarction, a transient ischemic attack, unstable angina, or sickle cell anemia. Moreover, the central nervous system ischemic condition may occur as result of the subject undergoing a surgical procedure. By way of example, the subject may be undergoing heart surgery, lung surgery, spinal surgery, brain surgery, vascular surgery, abdominal surgery, or organ transplantation surgery. The organ transplantation surgery may include heart, lung, pancreas, kidney or liver transplantation surgery. Moreover, the central nervous system ischemic condition may occur as a result of a trauma or injury to a part of the subject's body outside the central nervous system. By way of example, the trauma or injury may cause a degree of bleeding that significantly reduces the total volume of blood in the subject's body. Because of this reduced total volume, the amount of blood flow to the central nervous system is concomitantly reduced. By way of further example, the trauma or injury may also result in the formation of a vaso-occlusion that restricts blood flow to the central nervous system.

It is contemplated that the HAT inhibiting compounds may be employed to treat the central nervous system ischemic condition irrespective of the cause of the condition. In one embodiment, the ischemic condition results from a vaso-occlusion. The vaso-occlusion may be any type of occlusion, but is typically a cerebral thrombosis or an embolism. In a further embodiment, the ischemic condition may result from a hemorrhage. The hemorrhage may be any type of hemorrhage, but is generally a cerebral hemorrhage or a subararachnoid hemorrhage. In still another embodiment, the ischemic condition may result from the narrowing of a vessel. Generally speaking, the vessel may narrow as a result of a vasoconstriction such as occurs during vasospasms, or due to arteriosclerosis. In yet another embodiment, the ischemic condition results from an injury to the brain or spinal cord.

In yet another aspect, a HAT inhibiting compound may be administered to reduce infarct size of the ischemic core following a central nervous system ischemic condition. Moreover, a HAT inhibiting compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include one or more HAT inhibitors and one or more anti-neurodegeneration agents. For example, one or more HAT inhibiting compounds can be combined with an effective amount of one or more of: L-DOPA; a dopamine agonist; an adenosine A2A receptor antagonist; a COMT inhibitor; a MAO inhibitor, an N-NOS inhibitor, a sodium channel antagonist; a selective N-methyl D-aspartate (NMDA) receptor antagonist; an AMPA/kainate receptor antagonist; a calcium channel antagonist; a GABA-A receptor agonist; an acetyl-choline esterase inhibitor; a matrix metalloprotease inhibitor; a PARP inhibitor, an inhibitor of p38 MAP kinase or c-jun-N-terminal kinases; TPA; NDA antagonists; beta-interferons; growth factors; glutamate inhibitors; and/or as part of a cell therapy.

Exemplary N-NOS inhibitors include 4-(6-amino-pyridin-2-yl)-3-methoxyphenol 6-[4-(2-dimethylamino-ethoxy)-2-methoxy-phenyl]-pyridin-2-yl-amine, 6-[4-(2-dimethylamino-ethoxy)-2,3-dimet-hyl-phenyl]-pyridm-2-yl-amine, 6-[4-(2-pyrrolidinyl-ethoxy)-2,3-dimethyl-p-henyl]-pyridin-2-yl-amine, 6-[4-(4-(n-methyl)piperidinyloxy)-2,3-dimethyl-p-henyl]-pyridin-2-yl-amine, 6-[4-(2-dimethylaiuino-ethoxy)-3-methoxy-phenyl]-pyridin-2-yl-amine, 6-[4-(2-pyrrolidinyl-ethoxy)-3-methoxy-phenyl]-pyridin-2-yl-amine, 6-{4-[2-(6,7-dimethoxy-3,4-dihydro-1h-isoquinolin-2-yl)-ethoxy]-3-methoxy-phenyl}-pyridin-2-yl-amine, 6-{3-methoxy-4-[2-(4-phenethyl-piper-azin-1-yl)-ethoxy]-phenyl}-pyridin-2-yl-amine, 6-{3-methoxy-4-[2-(4-methyl-piperazin-1-yl)-ethoxy]-phenyl}-pyridin-2-yl-amine, 6-{4-[2-(4-dimethylamin-o-piperidin-1-yl)-ethoxy]-3-methoxy-phenyl}-pyridin-2-yl-amine, 6-[4-(2-dimethylamino-ethoxy)-3-ethoxy-phenyl]-pyridin-2-yl-amine, 6-[4-(2-pyrrolidinyl-ethoxy)-3-ethoxy-phenyl]-pyridin-2-yl-amine, 6-[4-(2-dimethylamino-ethoxy)-2-isopropyl-phenyl]-pyridin-2-yl-amine, 4-(6-amino-pyridin-yl)-3-cyclopropyl-phenol 6-[2-cyclopropyl-4-(2-dimethyl-lamino-ethoxy)-phenyl]-pyridin-2-yl-amine, 6-[2-cyclopropyl-4-(2-pyrrolidin-1-yl-ethoxy)-phenyl]-pyridin-2-yl-amine, 3-[3-(6-amino-IK iL.;; !./LU a o B/<′> 3 Q B JLB pyridin-2-yl)-4-cycl-opropyl-phenoxy]-pyrrolidine-1-carboxylic acid tert-butyl ester 6-[2-cyclopropyl-4-(1-methyl-pyrrolidin-3-yl-oxy)-phenyl]-pyridin-2-yl-amine, 4-(6-amino-pyridin-2-yl)-3-cyclobutyl-phenol 6-[2-cyclobutyl-4-(2-dime-thylamino-ethoxy)-phenyl]-pyridin-2-yl-amine, 6-[2-cyclobutyl-4-(2-pyrrolid-in-1-yl-ethoxy)-5 phenyl]-pyridin-2-yl-amine, 6-[2-cyclobutyl-4-(1-methyl-pyr-rolidin-3-yl-oxy)-phenyl]-pyridin-2-yl-amine, 4-(6-amino-pyridin-2-yl)-3-cy-clopentyl-phenol 6-[2-cyclopentyl-4-(2-dimethylamino-ethoxy)-phenyl]-pyrid-in-2-yl-amine, 6-[2-cyclopentyl-4-(2-pyrrolidin-lyl-ethoxy)-phenyl]-pyridin-2-yl-amine, 3-[4-(6-amino-pyridin-2-yl)-3-methoxy-phenoxy]-pyrrolidine-1-ca-rboxylic acid tert butyl ester 6-[4-(1-methyl-pyrrolidin-3-yl-oxy)-2-metho-xy-phenyl]-pyridin-2-yl-amine, 4-[4-(6-amino-pyridin-2-yl)-3-methoxy-phenoxy-]-piperidine-1-carboxylic acid tert butyl ester 6-[2-methoxy-4-(1-methyl-piperidin-4-yl-oxy)-phenyl]-pyridin-2-yl-amine, 6-[4-(allyloxy)-2-methoxy-ph-enyl]-pyridin-2-yl-amine, 4-(6-amino-pyridin-2-yl)-3-methoxy-6-allyl-phenol 12 and 4-(6-amino-pyridin-2-yl)-3-methoxy-2-allyl-phenol 13 4-(6-amino-pyridin-2-yl)-3-methoxy-6-propyl-phenol 6-[4-(2-dimethylamino-ethoxy)-2-methoxy-5-propyl-phenyl]-pyridin-yl-amine, 6-[2-isopropyl-4-([rho]yrrolidin-3-yl-oxy)-phenyl]-pyridin-2-yl-amine, 6-[2-isopropyl-4-(piperidin-3-yl-oxy)-phenyl]-pyridin-2-yl-amine, 6-[2-isopropyl-4-(1-methyl-azetidin-3-yl-oxy)-phenyl]-pyridin-2-yl-amine, 6-[2-isopropyl-4-(1-methyl-piperidin-4-yl-oxy)-phenyl]-pyridin-2-yl-amine, 6-[2-isopropyl-4-(1-methyl-pyrrolidin-3-yl-oxy)iphenyl]-pyridin-2-yl-amm-e 6-[2-isopropyl-4-(1-methyl-pyrrolidm-3-yl-oxy)-phenyl]-pyridin-2-yl-amine, 6-[2-isopropyl-4-(2-methyl-2-aza-bicyclo[2.2.1]hept-5-yl-oxy)-phenyl]-p-yridin-2-yl-amine, 6-[4-(2-dimethylamino-ethoxy)-2-methoxy-phenyl]-pyridin-2-yl-amine, 6-{4-[2-(benzyl-methyl-amino)-ethoxy]-2-methoxy-phenyl}-pyridin-2-yl-amine, 6-[2-methoxy-4-(2-pyrrolidin-1-yl-ethoxy)-phenyl]-pyridin-2-yl-amine, 2-(6-amino-pyridin-2-yl)-5-(2-dimethylamino-ethoxy)-phenol 2-[4-(6-amino-pyridin-2-yl)-3-methoxy-phenoxy]-acetamide 6-[4-(2-amino-ethoxy)-2-methoxy-phenyl]-pyridin-2-yl-amine, 6-{4-[2-(3,4-dihydro-1 h-isoquinolin-2-yl)-ethoxy]-2-methoxy-phenyl}-pyrid-in-2-yl-amine, 2-[4-(6-amino-pyridin-2-yl)-3-methoxy-phenoxy]-ethanol 6-{2-methoxy-4-[2-(2,2,6,6-tetramethyl-O piperidin-1-yl)-ethoxy]-phenyl}-py-ridin-2-yl-amine, 6-{4-[2-(2,5-dimethyl-pyrrolidin-1-yl)-ethoxy]-2-methoxy-phenyl}-pyridin-2-yl-amine, 6-{4-[2-(2,5-dimethyl-pyrrolidin-1-yl)-ethoxy]-2-methoxy-phenyl}-pyridin-2-yl-amine, 2-[4-(6-amino-pyridin-2-yl)-3-methoxy-phenoxy]-1-(2,2,6,6-tetramethyl-piperidin-1-yl)-ethanone 6-[2-methoxy-4-(1-methyl-pyrrolidin-2-yl-methoxy)-phenyl]-pyridin-2-yl-arnme, 6-[4-(2-dimethylatnino-ethoxy)-2-propoxy-phenyl]-pyridin-2-yl-amine, 6-{4-[2-(benzyl-methyl-amino)-ethoxy]-2-propoxy-phenyl}-pyridin-2-yl-amin-e 6-[4-(2-ethoxy-ethoxy)-2-methoxy-phenyl]-pyridin-2-yl-amine, 6-[4-(2-dimethylamino-ethoxy)-2-isopropoxy-phenyl]-pyridin-2-yl-amine, 6-[4-(2-ethoxy-ethoxy)-2-isopropoxy-phenyl]-5 pyridin-2-yl-amine, 6-[2-methoxy-4-(3-methyl-butoxy)-phenyl]-pyridin-2-yl-amine, 6-[4-(2-dimethylamino-ethoxy)-2Z-ethoxy-phenyl]-pyi-idin-2-yl-amine, 6-{4-[2-(benzyl-methyl-amino)-ethoxy]-2-ethoxy-phenyl}-pyridin-2-yl-amine, 6-[2-ethoxy-4-(3-methyl-butoxy)-plienyl]-pyridin-2-yl-amine, 1-(6-amino-3-aza-bicyclo[3.1.0]hex-3-yl)-2-[4-(6-amino-[rho]yridin-2-yl)-3-et-lioxy-phenoxy]-ethanone 6-[2-ethoxy-4-(2-pyrrolidine 1-yl-ethoxy)-phenyl]-py-ridin-2-yl-amine, 3-{2-[4-(6-amino-pyridin-2-yl)-3-ethoxy-phenoxy]-ethyl}-3-aza-bicyclo[3.1.0]hex-6-yl-amine, 1-(6-amino-3-aza-bicyclo[3.1.0]hex-3-yl)-2-[4-(6-amino-pyridin-2-yl)-3-methoxy-phenoxy]-ethanone 3-{2-[4-(6-amino-pyridin-2-yl)-3-methoxy-phenoxy]-ethyl)}-3-aza-bicyclo[3.-1.0]hex-6-yl-amine, 6-[2-isopropoxy-4-(2-pyrrolidin-1-yl-ethoxy)-phenyl]-py-ridin-2-yl-amine, 6-{4-[2-(benzyl-methyl-amino)-ethoxy]-2-isopropoxy-ph′enyl-}-pyridin-2-yl-amine, 6-[4-(2-dimethylamino-ethoxy)-2-methoxy-5-propyl-phen-yl]-pyridin-2-yl-amine, 6-[5-.allyl-4-(2-dimethylamino-ethoxy)-2-methoxy-phe-nyl]-pyridin-2-yl-ammne, 6-[5-allyl-2-methoxy-4-(2-pyrrolidin-1-yl-ethoxy)-phenyl]-pyridin-2-yl-amine, 6-[3-allyl-4-(2-dimethylamino-ethoxy)-2-methoxy-phenyl]-pyridin-2-yl-amine, 6-[2-methoxy-4-(pyrrolidin-3-yl-oxy)-phenyl]-p-yridin-2-yl-ammne, 6-[2-methoxy-4-(1-methyl-pyrrolidin-3-yl-oxy)-phenyl]-py-ridin-2-yl-amine, 6-[2-ethoxy-4-([rho]yrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(piperidin-4-yl-oxy)-phenyl]-pyridin-2-yl-amine, 6-[2-methoxy-4-(2,2,6,6-tetramethyl-piperidin-4-yl-oxy)-phenyl]-pyridin-2-yl-amine, 6-[2-isopropoxy-4-(pyrrolidin-3-yl-oxy)-phenyl]-pyridin-2-yl-amine, 3-[4-(6-amino-pyridin-2-yl)-3-methoxy-phenoxy]-azetidine-1-carboxylic acid tert-butyl ester 6-[4-(azetidin-3-yl-oxy)-2-methoxy-[rho]henyl]-pyridin-2-yl-amine, 6-[2-methoxy-4-(1-methyl-azetidin-3-yl-oxy)-phenyl]-pyridin-2-y-l-amine, 6-[2-isopropoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(pyrrolidin-3-yl-oxy)-phenyl]-pyridin-2-yl-amine, 6-[2-methoxy-4-(1-methyl-pyrrolidin-3-yl-oxy)-phenyl]-[rho]yridin-2-yl-amine, 6-[2-methoxy-4-(1-methyl-pyrrolidin-3-yl-oxy)-phenyl]-pyridm-2-yl-amine, 6-[2-methoxy-4-(2-methyl-2-aza-bicyclo[2.2.1]hept-5-yl-oxy)-phenyl]-[rho]yrid-in-2-yl-amine, 6-[2-methoxy-4-(1-methyl-piperidin-4-yl-oxy)-phenyl]-pyridin-2-yl-amine, 6-[4-(1-ethyl-piperidin-4-yl-oxy)-2-methoxy-phenyl]-pyridin-2-yl-amine, 6-[5-allyl-2-methoxy-4-(1-methyl-pyrrolidin-3-yl-oxy)-phenyl]-pyr-idin-2-yl-amine, 6-[4-(2-dimethylamino-ethoxy)-2,6-dimethyl-plienyl]-pyridin-2-yl-amine, 6-[2,6-dimethyl-4-(3-piperidin-1-yl-propoxy)-phenyl]-[rho]yridin-2-yl-amine, 6-[2,6-dimethyl-4-(2-py[pi]Olidin-1-yl-ethoxy)-phenyl]-5 pyridin-2-y-l-amine, 6-{2,6-dimethyl-4-[3-(4-methyl-piperazin-1-yl)-propoxy]-phenyl}-py-ridin-2-yl-araine, 6-[2,6-dimethyl-4-(2-moipholin-4-yl-ethoxy)-phenyl]-pyrid-in-2-yl-amine, 6-{4-[2-(benzyl-methyl-amino)-ethoxy]-2,6-dimethyl-phenyl}-p-yridin-2-yl-amine, 2-[4-(6-amino-pyridin-2-yl)-3,5-dimethyl-phenoxy]-acetam-ide 6-[4-(2-amino-ethoxy)-2,6-dimethyl-phenyl]-pyridin-2-yl-amine, 6-[2-isopropyl-4-(2-pyrrolidin-1-yl-ethoxy)-phenyl]-pyridin-2-yl-amine, 2-(2,5-dimethyl-pyrrolidin-1-yl)-6-[2-isopropyl-4-(2-pyrrolidin-1-yl-etho-xy)-phenyl]-pyridine 6-{4-[2-(3,5-dimethyl-piperidin-1-yl)-ethoxy]-2-isopr-opyl-phenyl}-pyridin-2-yl-amine, 6-[4-(2-dimethylamino-ethoxy)-2-isopropyl-phenyl]-pyridin-2-yl-amine, 6-[2-tert-butyl-4-(2-dimethylamino-ethoxy)-phen-yl]-pyridin-2-yl-amine, 6-[2-tert-butyl-4-(2-pyrrolidin-1-yl-ethoxy)-phenyl-]-pyridin-2-yl-amine, 6-[4-(2-pyrrolidinyl-ethoxy)-2,5-dimethyl-phenyl]-pyr-idin-2-yl-amine, 6-[4-(2-dimethylamino-ethoxy)-2,5-dimethyl-phenyl]-pyridin-2-yl-amine, 6-[4-(2-(4-phenethylpiperazin-1-yl)-ethoxy)-2,5-dimethyl-pheny-l]-pyridin-2-yl-amine, 6-[2-cyclopropyl-4-(2-dimethylamino-1-methyl-ethoxy)-phenyl]-pyridin-2-yl-amine, 6-[cyclobutyl-4-(2-dimethylamino-1-methyl-etho-xy)-phenyl]-pyridin-2-yl-ainine, 6-[4-(allyloxy)-2-cyclobutyl-phenyl]-pyridi-n-2-ylamine, 2-allyl-4-(6-amino-pyridin-2-yl)-3-cyclobutyl-phenol and 2-allyl-4-(6-amino-pyridin-2-yl)-5-cyclobutyl-phenol 4-(6-amino-pyridin-2-yl)-5-cyclobutyl-2-propyl-phenol 4-(6-amino-pyridin-2-yl)-3-cyclobutyl-2-propyl-phenol 6-[2-cyclobutyl-4-(2-dimethylamino-1-methyl-ethoxy)-5-piOpyl-phenyl]-pyri-din-2-yl-amine, 6-[2-cyclobutyl-4-(2-dimethylamino-1-methyl-ethoxy)-3-piOpy-1-phenyl]-pyridin-2-yl-amine, 6-[2-cyclobutyl-4-(2-dimethylamino-ethoxy)-5-propyl-phenyl]-pyridin-2-yl-amine, 6-[2-cyclobutyl-4-(2-dimethylamino-ethox-y)-3-propyl-phenyl]-pyridin-2-yl-amine, 6-[2-cyclobutyl-4-(1-methyl-pyrroli-din-3-yl-oxy)-5-propyl-phenyl]-pyridin-2-yl-amine, 6-[cyclobutyl-4-(1-methyl-1-pyr[tau]olidin-3-yl-oxy)-3-propyl-phenyl]-pyridin-2-yl-amine, 2-(4-benzyloxy-5-hydroxy-2-methoxy-phenyl)-6-(2,5-dimethyl-pyrrol-1-yl)-p-yridin 6-[4-(2-dimethylamino-ethoxy)-5-ethoxy-2-methoxy-phenyl]-pyridin-2-yl-amine, 6-[5-ethyl-2-methoxy-4-(1-methyl-piperidin-4-yl-oxy)-phenyl]-pyr-idin-2-yl-amine, 6-[5-ethyl-2-methoxy-4-(piperidin-4-yl-oxy)-phenyl]-pyridi-n-2-yl-amine, 6-[2,5-dimethoxy-4-(1-methyl-pyrrolidin-3-yl-oxy)-phenyl]-pyr-idin-2-yl-amine, 6-[4-(2-dimethylamino-ethoxy)-5-ethyl-2-methoxy-phenyl]-py-ridin-2-yl-amine.

Exemplary NMDA receptor antagonist include (+)-(1S,2S)-1-(4-hydroxy-phenyl)-2-(4-hydroxy-4-phenylpiperidino)-1-pro-panol, (1S,2S)-1-(4-hydroxy-3-methoxyphenyl)-2-(4-hydiOxy-4-phenylpiperi-dino)-1-propanolJ (3R,4S)-3-(4-(4-fluorophenyl)-4-hydiOxypiperidin-1-yl-)-chroman-4,7-diol, (1R*,2R*)-1-(4-hydroxy-3-methylphenyl)-2-(4-(4-fluoro-phenyl)-4-hydiOxypiperidin-1-yl)-propan-1-ol-mesylate or a pharmaceutically acceptable acid addition salt thereof.

Exemplary dopamine agonist include ropininole; L-dopa decarboxylase inhibitors such as carbidopa or benserazide, bromocriptine, dihydroergocryptine, etisulergine, AF-14, alaptide, pergolide, piribedil; dopamine D1 receptor agonists such as A-68939, A-77636, dihydrexine, and SKF-38393; dopamine D2 receptor agonists such as carbergoline, lisuride, N-0434, naxagolide, PD-118440, pramipexole, quinpirole and ropinirole; dopamine/[beta]-adrenegeric receptor agonists such as DPDMS and dopexamine; dopamine/5-HT uptake inhibitor/5-HT-1 A agonists such as roxindole; dopamine/opiate receptor agonists such as NIH-10494; [alpha]2-adrenergic antagonist/dopamine agonists such as terguride; [alpha]2-adrenergic antagonist/dopamine D2 agonists such as ergolines and talipexole; dopamine uptake inhibitors such as GBR-12909, GBR-13069, GYKI-52895, and NS-2141; monoamine oxidase-B inhibitors such as selegiline, N-(2-butyl)-N-methylpropargylamine, N-methyl-N-(2-pentyl)propargylamine, AGN-1133, ergot derivatives, lazabemide, LU-53439, MD-280040 and mofegiline; and COMT inhibitors such as CGP-28014.

Exemplary acetyl cholinesterase inhibitors include donepizil, 1-(2-methyl-IH-benzimida-zol-5-yl)-3-[1-(phenylmethyl)-4-piperidinyl]-1-propanone; 1-(2-phenyl-1H-benzimidazol-5-yl)-3-[1-(phenylmethyl)-4-piperidinyl]-1-pr-opanone; 1-(1-ethyl-2-methyl-1H-benzimidazol-5-yl)-3-[1-(phenylmethyl)-4-piperidinyl]-1-propanone; 1-(2-methyl-6-benzothiazolyl)-3-[1-(phenylmethyl)-4-piperidinyl]-1-propanone; 1-(2-methyl-6-benzothiazolyl)-3-[1-[(2-methyl-4-thiazolyl)methyl]-4-piperidinyl]-1-propanone; 1-(5-methyl-benzo[b]thie-n-2-yl)-3-[1-(phenylmethyl)-4-piperidinyl]-1-propanone; 1-(6-methyl-benzo[b]thien-2-yl)-3-[1-(phenylmethyl)-4-piperidinyl]-1-prop-anone; 1-(3,5-dimethyl-benzo[b]thien-2-yl)-3-[1-(phenylmethyl)-4-piperidin-yl]-1-propanone; 1-(benzo[b]thien-2-yl)-3-[1-(phenylmethyl)-4-piperidinyl]-1-propanone; 1-(benzofuran-2-yl)-3-[1-(phenylmethyl)-4-piperidinyl]-1-pro-panone; 1-(1-P IC T/U S O 6./3 O S .1 S phenylsulfonyl-6-methyl-indol-2-yl)-3-[1-(phenylmethyl)-4-pip-eridinyl]-1-propanone; 1-(6-methyl-indol-2-yl)-3-[1-(phenylmethyl)-4-piper-idinyl]-1-propanone; 1-(1-phenylsulfonyl-5-amino-indol-2-yl)-3-[1-(phenylm-ethyl)-4-piperidinyl]-1-propanone; 1-(5-amino-indol-2-yl)-3-[1-(phenylmet-hyl)-4-piperidinyl]-1-propanone; and 1-(5-5 acetylamino-indol-2-yl)-3-[1-(ph-enylmethyl)-4-piperidinyl]-1-propanone. 1-(6-quinolyl)-3-[1-(phenylmetliyl)-4-piperidinyl]-1-propanone; 1-(5-indolyl)-3-[1-(phenylmethyl)-4-piperidiny-l]-1-propanone; 1-(5-benzthienyl)-3-[1-(phenylmethyl)-4-piperidinyl]-1-pro-panone; 1-(6-quinazolyl)-3-[1-(phenylmethyl)-4-[rho]iperidinyl]-1-propanone; 1-(6-benzoxazolyl)-3-[1-(phenylmethyl)-4-piperidinyl]-1-propanone; 1-(5-benzofuryl)-3-[1-(phenylmethyl)-4-piperidinyl]-1-propanone; 1-(5-methyl-benzimidazol-2-yl)-3-[1-([rho]henylmethyl)-4-[rho]piperidinyl]-1-piOpa-none; 1-(6-methyl-benzimidazol-2-yl)-3-[1-(phenylmethyl)-4-piperidinyl]-1-propanone; 1-(5-chloro-benzo[b]thien-2-yl)-3-[1-(phenylmethyl)-4-piperidin-yl]-1-propanone; 1-(5-azaindol-2-yl)-3-[1-(phenylmethyl)-4-piperidinyl]-1-p-ropanone; 1-(6-azabenzo[b]thien-2-yl)-3-[1-(phenylmethyl)-4-piperidinyl]-1-propanone; 1-(1H-2-oxo-pyrrolo[2′,3′,5,6]benzo[b]thieno-2-yl)-3-[1-(phenylmethyl)-4-piperidinyl]-1-propanone; 1-(6-methyl-benzothiazol-2-yl)-3-[1-(phenylmethyl)-4-piperidinyl]-1-propanone; 1-(6-methoxy-indol-2-yl)-3-[1-(phenylmethyl)-4-piperidinyl]-1-propanone; 1-(6-methoxy-benzo[b]thien-2-yl)-3-[1-(phenylmethyl)-4-piperidinyl]-1-pro-panone; 1-(6-acetylamino-benzo[b]thien-2-yl)-3-[1-(phenylmethyl)-4-piperid-inyl]-1-pro[rho]anone; 1-(5-acetylamino-benzo[b]thien-2-yl)-3-[1-(phenylmethyl-)-4-piperidinyl]-1-propanone; 6-hydroxy-3-[2-[1-(phenylmethyl)-4-piperidin-yl]ethyl]-1,2-benzisoxazole; 5-methyl-3-[2-[1-(phenylmethyl)-4-piperidinyl-]ethyl]-1,2-benzisoxazole; 6-methoxy-3[2-[1(phenylmethyl)-4-piperidinyl]et-hyl]-1,2-benzisoxazole; 6-acetamide-3-[2-[1-(phenylmethyl)-4-piperidinyl]-ethyl]-1,2-benzisoxazole; 6-amino-3-[2-[1-(phenymethyl)-4-piperidinyl]ethyl-1]-1,2-benzisoxazole; 6-(4-morpholinyl)˜3-[2-[1-(phenylmethyl)-4-piperidin-yl]ethyl]-1,2-benzisoxazole; 5,7-dihydro-3-[2-[1-(phenylmethyl)-4-piperidi-nyl]ethyl]-6H-pyrrolo[4,5-f]-1,2-benzisoxazol-6-one; 3-[2-[1-(phenylmethyl)-4-piperidinyl]ethyl]-1,2-benzisothiazole; 3-[2-[1-(phenylmethyl)-4-piperidinyl]ethenyl]-1,2-benzisoxazole; 6-phenylamino-3-[2-[1-(phenylmethyl)-4-piperidinyl]ethyl]-1,2,-benzisoxaz-ole; 6-(2-thiazoly)-3-[2-[1-(phenylmethyl)-4-piperidinyl]ethyl]-1,2-benzis-oxazole; 6-(2-oxazolyl)-3-[2-[1-(phenylmethyl)-4-piperidinyl]ethyl]-1,2-be-nzisoxazole; 6-pyrrolidinyl-3-[2-[1-(phenylmethyl)-4-piperidinyl]ethyl]-1,-2-benzisoxazole; 5,7-dihydro-5,5-dimethyl-3-[2-[(phenylmethyl)-4-piperid-inyl]ethyl]-6H-pyrrolo[4,5-f]-1,2-benzisoxazole-6-one; 6,8-dihydro-3-[2-[1-(phenylmethyl)-4-piperidinyl]ethyl]-7H-pyrrolo[5,4-g]-1,2-benzisoxazole-7-one; 3-[2-[1-(phenylmethyl)-4-piperidinyl]ethyl]-5,6,-8-trihydro-7H-isoxazolo[4,5-g]-quinolin-7-one; 1-benzyl-4-((5,6-dimethoxy-1-indanon)-2-5 yl)methylpiperidine, 1-benzyl-4-((5,6-dimethoxy-1-indanon)-2-ylidenyl)methylpiperidine, 1-benzyl-4-((5-methoxy-1-indanon)-2-yl)methylp-piperidine, 1-benzyl-4-((5,6-diethoxy-1-indanon)-2-yl)methylpiperidine, 1-benzyl-4-((5,6-methnylenedioxy-1-indanon)-2-yl)methylpiperidine, 1-(m-nitrobenzyl)-4-((5,6-dimethoxy-1-indanon)-2-yl)methylpiperidine, 1-cyclohexymethyl-4-((5,6-dimethoxy-1-indanon)-2-yl)methylpiperidine, 1-(m-florobenzyl)-4-((5,6-dimethoxy-1-indanon)-2-yl)methylpiperidine, 1-benzyl-4-((5,6-dimethoxy-1-indanon)-2-yl)propylpiperidine, and 1-benzyl-4-((5-isopropoxy-6-methoxy-1-indanon)-2-yl)methylpiperidine.

Exemplary calcium channel antagonists include diltiazem; omega-conotoxin GVIA, methoxyverapamil, amlodipine, felodipine, lacidipine, and mibefradil.

Exemplary GABA-A receptor modulators include clomethiazole; IDDB; gaboxadol (4,5,6,7-tetrahydroisoxazolo[5,4-c]pyridin-3-ol); ganaxolone (3[alpha]-hydroxy-3[beta]-methyl-5[alpha]-pregnan-20-one); fengabine (2-[(butylimino)-(2-chlorophenyl)methyl]-4-chlorophenol); 2-(4-methoxyphenyl)-2,5,6,7,8,9-hexahydro-pyrazolo[4,3-c]cinnolin-3-one; 7-cyclobutyl-6-(2-methyl-2H-1,2,4-triazol-3-ylmethoxy)-3-phenyl-1,2,4-triazolo[4,3-b]pyridazine; (3-fluoro-4-methylphenyl)-N-({-1-[(2-methylphenyl)methyl]-benzimidazol-2-yl}methyl)-N-pentylcarboxamide; and 3-(aminomethyl)-5-methylhexanoic acid.

Exemplary potassium channel openers include diazoxide, flupirtine, pinacidil, levcromakalim, rilmakalim, chromakalim, PCO-400 and SKP-450 (2-[2″(T<1>, 3″-dioxolone)-2-methyl]-4-(2′-oxo-r-pyrrolidinyl)-6-nitro-2H-1-benzopyra-n).

Exemplary AMPA/kainate receptor antagonists include 6-cyano-7-nitroquinoxalin-2,3-di-one (CNQX); 6-nitro-7-sulphamoylbenzo[f]quinoxaline-2,3-dione (NBQX); 6,7-dinitroquinoxaline-2,3-dione (DNQX); 1-(4-aminophenyl)-4-methyl-7,8-m-ethylenedioxy-5H-2,3-benzodiazepine hydrochloride; and 2,3-dihydroxy-6-nitro-7-sulfamoylbenzo-[f]quinoxaline.

Exemplary sodium channel antagonists include ajmaline, procainamide, flecamide and riluzole.

Exemplary matrix-metalloprotease inhibitors include 4-[4-(4-fluorophenoxy)benzenesulfon-ylamino]tetrahydropyran-4-carboxylic acid hydroxyamide; 5-Methyl-5-(4-(4′-fluoiOplienoxy)-phenoxy)-[rho]yrimidine-2,4,6-trione; 5-n-Butyl-5-(4-(4′-fluorophenoxy)-[rho]henoxy)-pyrimidine-2,4,6-trione and prinomistat. 5 PoIy(ADP ribose) polymerase (PARP) is an abundant nuclear enzyme which is activated by DNA strand single breaks to synthesize poly (ADP ribose) from NAD. Under normal conditions, PARP is involved in base excision repair caused by oxidative stress via the activation and recruitment of DNA repair enzymes in the nucleus. Thus, PARP plays a role in cell necrosis and DNA repair. PARP also participates in regulating cytokine expression that mediates inflammation. Under conditions where DNA damage is excessive (such as by acute excessive exposure to a pathological insult), PARP is over-activated, resulting in cell-based energetic failure characterized by NAD depletion and leading to ATP consumption, cellular necrosis, tissue injury, and organ damage/failure. PARP is thought to contribute to neurodegeneration by depleting nicotinamide adenine dinucleotide (NAD+) which then reduces adenosine triphosphate (ATP; Cosi and Marien, Ann. N.Y. Acad. ScL, 890:227, 1999) contributing to cell death which can be prevented by PARP inhibitors. Exemplory PARP inhibitors can be found in Southan and Szabo, Current Medicinal Chemistry, 10:321, 2003.

Exemplary inhibitors of p38 MAP kinase and c-jun-N-terminal kinases include pyridyl imidazoles, such as PD 169316, isomeric PD 169316, SB 203580, SB 202190, SB 220026, and RWJ 67657. Others are described in U.S. Pat. No. 6,288,089, and incorporated by reference herein.

In an exemplary embodiment, a combination therapy for treating or preventing MS comprises a therapeutically effective amount of one or more HAT modulating compounds that decrease the level and/or activity of a HAT and one or more of Avonex© (interferon beta-Ia), Tysabri® (natalizumab), or Fumaderm (BG-12/Oral Fumarate).

In another embodiment, a combination therapy for treating or preventing diabetic neuropathy or conditions associated therewith comprises a therapeutically effective amount of one or more HAT-modulating compounds that decreases the level and/or activity of a HAT and one or more of tricyclic antidepressants (TCAs) (including, for example, imipramine, amytriptyline, desipramine and nortriptyline), serotonin reuptake inhibitors (SSRIs) (including, for example, fluoxetine, paroxetine, sertralene, and citalopram) and antiepileptic drags (AEDs) (including, for example, gabapentin, carbamazepine, and topimirate)

In another embodiment, the invention provides a method for treating or preventing a polyglutamine disease using a combination comprising at least one HAT inhibiting compound and at least one HDAC III inhibitor. Examples of HDAC I/II inhibitors include hydroxamic acids, cyclic peptides, benzamides, short-chain fatty acids, and depudecin.

Examples of hydroxamic acids and hydroxamic acid derivatives, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), valproic acid and pyroxamide. TSA was isolated as an antifungi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme 15 is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature: a polar hydroxamic terminal linked through a hydrophobic methylene spacer (e.g. 6 carbon at length) to another polar site which is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and depsipeptide. Trapoxin A is a cyclic tetrapeptide that contains a 2-amino-8-oxo-9,10-epoxy-decanoyl (AOE) moiety. Kijima et al. (1993) J. Biol. Chem. 268:22429-22435. Apicidin is a fungal metabolite that exhibits potent, broad-spectrum antiprotozoal activity and inhibits HDAC activity at nanomolar concentrations. Darkin-Rattray et al. (1996) Proc. Natl. Acad. Sci. USA. 93; 13143-13147. Depsipeptide is isolated from Chromobacterium violaceum, and has been shown to inhibit HDAC activity at micromolar concentrations.

Examples of benzamides include but are not limited to MS-27-275. Saito et al. (1990) Proc. Natl. Acad. Sci. USA. 96:4592-4597.

Examples of short-chain fatty acids include but are not limited to butyrates (e.g., butyric acid, arginine butyrate and phenylbutyrate (PB)). Newmark et al. (1994) Cancer Lett. 78:1-5; and Carducci et al. (1997) Anticancer Res. 17:3972-3973. In addition, depudecin which has been shown to inhibit HDAC at micromolar concentrations (Kwon et al. (1998) Proc. Natl. Acad. Sci. 5 USA. 95:3356-3361) also falls within the scope of histone deacetylase inhibitor as described herein.

Blood Coagulation Disorders

In other aspects, HAT-modulating compounds that decrease the level and/or activity of a HAT can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. After initiation of clotting, blood coagulation proceeds through the sequential activation of certain plasma proenzymes to their enzyme forms (see, for example, Coleman, R. W. et al. (eds.) Hemostasis and Thrombosis, Second Edition, (1987)). These plasma glycoproteins, including Factor XII, Factor XI, Factor IX, Factor X, Factor VII, and prothrombin, are zymogens of serine proteases. Most of these blood clotting enzymes are effective on a physiological scale only when assembled in complexes on membrane surfaces with protein cofactors such as Factor VIII and Factor V. Other blood factors modulate and localize clot formation, or dissolve blood clots. Activated protein C is a specific enzyme that inactivates procoagulant components. Calcium ions are involved in many of the component reactions. Blood coagulation follows either the intrinsic pathway, where all of the protein components are present in blood, or the extrinsic pathway, where the cell-membrane protein tissue factor plays a critical role. Clot formation occurs when fibrinogen is cleaved by thrombin to form fibrin. Blood clots are composed of activated platelets and fibrin.

Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place. It involves a cascade of complicated and regulated biochemical reactions between circulating blood-proteins (coagulation factors), blood cells (in particular platelets), and elements of an injured vessel wall. Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering a HAT-modulating compound that decreases the level and/or activity of a HAT. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. Examples of thrombotic disorders include, but are not limited to, thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, e.g., unstable angina, disseminated intravascular coagulation, thrombotic thrombocytopenic purpura, cancer metastasis, sickle cell disease, glomerular nephritis, and drug induced thrombocytopenia (including, for example, heparin induced thrombocytopenia). In addition, HAT-modulating compounds that decrease the level and/or activity of a HAT may be administered to prevent thrombotic events or to prevent re-occlusion during or after therapeutic clot lysis or procedures such as angioplasty or surgery.

In another embodiment, a combination drug regimen may include drugs or compounds for the treatment or prevention of blood coagulation disorders or secondary conditions associated with these conditions. Thus, a combination drug regimen may include one or more HAT-modulating compounds that decrease the level and/or activity of a HAT and one or more anti-coagulation or anti-thrombosis agents.

For example, one or more HAT-modulating compounds can be combined with an effective amount of one or more of: aspirin, heparin, and oral Warfarin that inhibits Vit K-dependent factors, low molecular weight heparins that inhibit factors X and II, thrombin inhibitors, inhibitors of platelet GP IIbIIa receptors, inhibitors of tissue factor (TF), inhibitors of human von Willebrand factor, inhibitors of one or more factors involved in hemostasis (in particular in the coagulation cascade). In addition, HAT-modulating compounds that decrease the level and/or activity of a HAT can be combined with thrombolytic agents, such as t-PA, streptokinase, reptilase, TNK-t-PA, and staphylokinase.

Weight Control

In another aspect, HAT-modulating compounds that decrease the level and/or activity of a HAT may be used for treating or preventing weight gain or obesity in a subject. For example, HAT-modulating compounds that decrease the level and/or activity of a HAT may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, HAT-modulating compounds that decrease the level and/or activity of a HAT may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Stunkard A J, Wadden T A. (Editors) Obesity: theory and therapy, Second Edition. New York: Raven Press, 1993. Finally, patients with AIDS can develop lipodystrophy or insulin resistance in response to combination therapies for AIDS.

In another embodiment, HAT-modulating compounds that decrease the level and/or activity of a HAT may be used for inhibiting adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used for treating or preventing obesity.

In other embodiments, HAT-modulating compounds that decrease the level and/or activity of a HAT may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

In other embodiments, a HAT-modulating compound that increases the level and/or activity of a HAT may be used to stimulate appetite and/or weight gain. A method may comprise administering to a subject, such as a subject in need thereof, a pharmaceutically effective amount of a HAT-modulating agent that increases the level and/or activity of a HAT. A subject in need of such a treatment may be a subject who has cachexia or may be likely to develop cachexia. A combination of agents may also be administered. Methods for stimulating fat accumulation in cells may be used in vitro, to establish cell models of weight gain, which maybe used, e.g., for identifying other drugs that prevent weight gain. Also provided are methods for modulating adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used to modulate obesity. A method for stimulating adipogenesis may comprise contacting a cell with a HAT-modulating agent that increases the level and/or activity of a HAT.

In another embodiment, the invention provides methods of decreasing fat or lipid metabolism in a subject by administering a HAT-modulating compound that increases the level and/or activity of a HAT. The method includes administering to a subject an amount of a HAT-modulating compound, e.g., in an amount effective to decrease mobilization of fat to the blood from WAT cells and/or to decrease fat burning by BAT cells.

Methods for promoting appetite and/or weight gain may include, for example, prior identifying a subject as being in need of decreased fat or lipid metabolism, e.g., by weighing the subject, determining the BMI of the subject, or evaluating fat content of the subject or HAT activity in cells of the subject. The administering can include one or more dosages, e.g., delivered in boluses or continuously. Monitoring can include evaluating a hormone or a metabolite. Exemplary hormones include leptin, adiponectin, resistin, and insulin. Exemplary metabolites include triglyercides, cholesterol, and fatty acids.

In one embodiment, a HAT-modulating compound that increases the level and/or activity of a HAT may be used to modulate (e.g., increase) the amount of subcutaneous fat in a tissue, e.g., in facial tissue or in other surface-associated tissue of the neck, hand, leg, or lips. The HAT-modulating compound may be used to increase the rigidity, water retention, or support properties of the tissue. For example, the HAT-modulating compound can be applied topically, e.g., in association with another agent, e.g., for surface-associated tissue treatment. The HAT-modulating compound may also be injected subcutaneously, e.g., within the region where an alteration in subcutaneous fat is desired.

A method for modulating weight may further comprise monitoring the weight of the subject and/or the level of modulation of HATs, for example, in adipose tissue.

In an exemplary embodiment, HAT-modulating compounds that decrease the level and/or activity of a HAT may be administered as a combination therapy for treating or preventing weight gain or obesity. For example, one or more HAT-modulating compounds that decrease the level and/or activity of a HAT may be administered in combination with one or more anti-obesity agents. Exemplary anti-obesity agents include, for example, phenylpropanolamine, ephedrine, pseudoephedrine, phentermine, a cholecystokinin-A agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (leptin), a leptin analog, a leptin receptor agonist, a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin and ciliary neurotrophic factors such as Axokine.

In another embodiment, HAT-modulating compounds that decrease the level and/or activity of a HAT may be administered to reduce drug-induced weight gain. For example, a HAT-modulating compound that decreases the level and/or activity of a HAT may be administered as a combination therapy with medications that may stimulate appetite or cause weight gain, in particular, weight gain due to factors other than water retention. Examples of medications that may cause weight gain, include for example, diabetes treatments, including, for example, sulfonylureas (such as glipizide and glyburide), thiazolidinediones (such as pioglitazone and rosiglitazone), meglitinides, nateglinide, repaglinide, sulphonylurea medicines, and insulin; anti-depressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy, lithium carbonate; valproic acid; carbamazepine; chlorpromazine; thiothixene; beta blockers (such as propranolo); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, HAT-modulating compounds that decrease the level and/or activity of a HAT may be administered as part of a smoking cessation program to prevent weight gain or reduce weight already gained.

### Metabolic Disorders/Diabetes

In another aspect, HAT-modulating compounds that decrease the level and/or activity of a HAT may be used for treating or preventing a metabolic disorder, such as insulin-resistance, a pre-diabetic state, type II diabetes, and/or complications thereof. Administration of a HAT-modulating compounds that decreases the level and/or activity of a HAT may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, HAT-modulating compounds that decrease the level and/or activity of a HAT may be administered as a combination therapy for treating or preventing a metabolic disorder. For example, one or more HAT-modulating compounds that decrease the level and/or activity of a HAT may be administered in combination with one or more anti-diabetic agents. Exemplary antidiabetic agents include, for example, an aldose reductase inhibitor, a glycogen phosphorylase inhibitor, a sorbitol dehydrogenase inhibitor, a protein tyrosine phosphatase IB inhibitor, a dipeptidyl protease inhibitor, insulin (including orally bioavailable insulin preparations), an insulin mimetic, metformin, acarbose, a peroxisome proliferator-activated receptor-[gamma](PPAR-[gamma]) ligand such as troglitazone, rosaglitazone, pioglitazone or GW-1929, a sulfonylurea, glipazide, glyburide, or chlorpropamide wherein the amounts of the first and second compounds result in a therapeutic effect. Other anti-diabetic agents include a glucosidase inhibitor, a glucagon-like peptide-1 (GLP-I), insulin, a PPAR [alpha]/[gamma]dual agonist, a meglitimide and an [alpha]P2 inhibitor. In an exemplary embodiment, an anti-diabetic agent may be a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrolidine) or MK-04301 from Merck (see e.g., Hughes et al., Biochemistry 38: 11597-603 (1999)).

### Inflammatory Diseases

In other aspects, HAT-modulating compounds that decrease the level and/or activity of a HAT can be used to treat or prevent a disease or disorder associated with inflammation. HAT-modulating compounds that decrease the level and/or activity of a HAT may be administered prior to the onset of, at, or after the initiation of inflammation. When used prophylactically, the compounds are preferably provided in advance of any inflammatory response or symptom. Administration of the compounds may prevent or attenuate inflammatory responses or symptoms. Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, HAT-modulating compounds that decrease the level and/or activity of a HAT may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, HAT-modulating compounds that decrease the level and/or activity of a HAT may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more HAT-modulating compounds that decrease the level and/or activity of a HAT may be taken alone or in combination with other compounds useful for treating or preventing inflammation. Exemplary anti-inflammatory agents include, for example, steroids (e.g., Cortisol, cortisone, fludrocortisone, prednisone, 6[alpha]-methylprednisone, triamcinolone, betamethasone or dexamethasone), nonsteroidal antiinflammatory drugs (NSAIDS (e.g., aspirin, acetaminophen, tolmetin, ibuprofen, mefenamic acid, piroxicam, nabumetone, rofecoxib, celecoxib, etodolac or nimesulide). In another embodiment, the other therapeutic agent is an antibiotic (e.g., vancomycin, penicillin, amoxicillin, ampicillin, cefotaxime, ceftriaxone, cefixime, rifampinmetronidazole, doxycycline or streptomycin). In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine). In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, [alpha]-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, caiprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide, cyclazocine, deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocoitolone, fluoresone, fluorometholone, fluperolone, flupirtine, fluprednidene, fluprednisolone, fluproquazone, flurandrenolide, flurbiprofen, fluticasone, formocortal, fosfosal, gentisic acid, glafenine, glucametacin, glycol salicylate, guaiazulene, halcinonide, halobetasol, halometasone, haloprednone, heroin, hydrocodone, hydro cortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate, myrophine, nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac.

In an exemplary embodiment, a HAT-modulating compound that decreases the level and/or activity of a HAT may be administered with a selective COX-2 inhibitor for treating or preventing inflammation. Exemplary selective COX-2 inhibitors include, for example, deracoxib, parecoxib, celecoxib, valdecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(triflu-oromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl)-1H-pyrazol-1-yl]benzenesulfonamide, tert-butyl 1 benzyl-4-[(4-oxopiperidin-1-yl}sulfonyl]piperidine-4-carboxylate, 4-[5-(phenyl)-3-(trifluoromethyl)-1H-pyrazol-1-yl]benzenesulfonamide, salts and prodrugs thereof.

Flushing

In another aspect, HAT-modulating compounds that decrease the level and/or activity of a HAT may be used for reducing the incidence or severity of flushing and/or hot flashes which are symptoms of a disorder. For instance, the subject method includes the use of HAT-modulating compounds that decrease the level and/or activity of a HAT, alone or in combination with other agents, for reducing incidence or severity of flushing and/or hot flashes in cancer patients. In other embodiments, the method provides for the use of HAT-modulating compounds that decrease the level and/or activity of a HAT to reduce the incidence or severity of flushing and/or hot flashes in menopausal and post-menopausal woman.

In another aspect, HAT-modulating compounds that decrease the level and/or activity of a HAT may be used as a therapy for reducing the incidence or severity of flushing and/or hot flashes which are side-effects of another drug therapy, e.g., drug-induced flushing. In certain embodiments, a method for treating and/or preventing drug-induced flushing-comprises administering to a patient in need thereof a formulation comprising at least one flushing inducing compound and at least one HAT-modulating compound that decreases the level and/or activity of a HAT. In other embodiments, a method for treating drug induced flushing comprises separately administering one or more compounds that induce flushing and one or more HAT-modulating compounds, e.g., wherein the HAT-modulating compound and flushing inducing agent have not been formulated in the same compositions. When using separate formulations, the HAT-modulating compound maybe administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, HAT-modulating compounds that decrease the level and/or activity of a HAT may be used to reduce flushing side effects of a vasodilator or an antilipemic agent (including anticholesteremic agents and lipotropic agents). In an exemplary embodiment, a HAT-modulating compound that decreases the level and/or activity of a HAT may be used to reduce flushing associated with the administration of niacin.

Nicotinic acid, 3-pyridinecarboxylic acid or niacin, is an antilipidemic agent that is marketed under, for example, the trade names Nicolar®, SloNiacin®, Nicobid® 5 and Time Release Niacin®. Nicotinic acid has been used for many years in the treatment of lipidemic disorders such as hyperlipidemia, hypercholesterolemia and atherosclerosis. This compound has long been known to exhibit the beneficial effects of reducing total cholesterol, low density lipoproteins or “LDL cholesterol,” triglycerides and apolipoprotein a (Lp(a)) in the human body, while increasing desirable high density lipoproteins or “HDL cholesterol”.

Typical doses range from about 1 gram to about 3 grams daily. Nicotinic acid is normally administered two to four times per day after meals, depending upon the dosage form selected. Nicotinic acid is currently commercially available in two dosage forms. One dosage form is an immediate or rapid release tablet which should be administered three or four times per day. Immediate release (“IR”) nicotinic acid formulations generally release nearly all of their nicotinic acid within about 30 to 60 minutes following ingestion. The other dosage form is a sustained release form which is suitable for administration two to four times per day. In contrast to IR formulations, sustained release (“SR”) nicotinic acid formulations are designed to release significant quantities of drug for absorption into the blood stream over specific timed intervals in order to maintain therapeutic levels of nicotinic acid over an extended period such as 12 or 24 hours after ingestion.

As used herein, the term “nicotinic acid” is meant to encompass nicotinic acid or a compound other than nicotinic acid itself which the body metabolizes into nicotinic acid, thus producing essentially the same effect as nicotinic acid. Exemplary compounds that produce an effect similar to that of nicotinic acid include, for example, nicotinyl alcohol tartrate, d-glucitol hexanicotinate, aluminum nicotinate, niceritrol and d,l-alpha-tocopheryl nicotinate. Each such compound will be collectively referred to herein as “nicotinic acid.”

In another embodiment, the invention provides a method for treating and/or preventing hyperlipidemia with reduced flushing side effects. The method comprises the steps of administering to a subject in need thereof a therapeutically effective amount of nicotinic acid and a HAT-modulating compound that decreases the level and/or activity of a HAT in an amount sufficient to reduce flushing. In an exemplary embodiment, the nicotinic acid and/or HAT-modulating compound may be administered nocturnally.

In another representative embodiment, the method involves the use of HAT-modulating compounds that decrease the level and/or activity of a HAT to reduce flushing side effects of raloxifene. Raloxifene acts like estrogen in certain places in the body, but is not a hormone. It helps prevent osteoporosis in women who have reached menopause. Osteoporosis causes bones to gradually grow thin, fragile, and more likely to break. Evista slows down the loss of bone mass that occurs with menopause, lowering the risk of spine fractures due to osteoporosis. A common side effect of raloxifene is hot flashes (sweating and flushing). This can be uncomfortable for women who already have hot flashes due to menopause.

In another representative embodiment, the method involves the use of HAT-modulating compounds that decrease the level and/or activity of a HAT to reduce flushing side effects of antidepressants or anti-psychotic agent. For instance, HAT-modulating compounds that decrease the level and/or activity of a HAT can be used in conjunction (administered separately or together) with a serotonin reuptake inhibitor, a 5HT2 receptor antagonist, an anticonvulsant, a norepinephrine reuptake inhibitor, an [alpha]-adrenoreceptor antagonist, an NK-3 antagonist, an NK-I receptor antagonist, a PDE4 inhibitor, an Neuropeptide Y5 Receptor Antagonists, a D4 receptor antagonist, a 5HT IA receptor antagonist, a 5HT ID receptor antagonist, a CRF antagonist, a monoamine oxidase inhibitor, or a sedative-hypnotic drag.

In certain embodiments, HAT-modulating compounds that decrease the level and/or activity of a HAT may be used as part of a treatment with a serotonin reuptake inhibitor (SRI) to reduce flushing. In certain preferred embodiments, the SRI is a selective serotonin reuptake inhibitor (SSRI), such as a fluoxetinoid (fluoxetine, norfluoxetine) or a nefazodonoid (nefazodone, hydroxynefazodone, oxonefazodone). Other exemplary SSRFs include duloxetine, venlafaxine, milnacipran, citalopram, fluvoxamine, paroxetine and sertraline. The HAT-modulating compound that decreases the level and/or activity of a HAT can also be used as part of a treatment with sedative-hypnotic drag, such as selected from the group consisting of a benzodiazepine (such as alprazolam, chlordiazepoxide, clonazepam, chlorazepate, clobazam, diazepam, halazepam, lorazepam, oxazepam and prazepam), Zolpidem, and barbiturates. In still other embodiments, a HAT-modulating compound that decreases BOS—the level and/or activity of a HAT may be used as part of a treatment with a 5-HT1A receptor partial agonist, such as selected from the group consisting of buspirone, flesinoxan, gepirone and ipsapirone. HAT-modulating compounds that decrease the level and/or activity of a HAT can also used as part of a treatment with a norepinephrine reuptake inhibitor, such as selected from tertiary amine tricyclics and secondary amine tricyclics. Exemplary tertiary amine tricyclic include amitriptyline, clomipramine, doxepin, imipramine and trimipramine. Exemplary secondary amine tricyclic include amoxapine, desipramine, maprotiline, nortriptyline and protriptyline. In certain embodiments, HAT-modulating compounds that decrease the level and/or activity of a HAT may be used as part of a treatment with a monoamine oxidase inhibitor, such as selected from the group consisting of isocarboxazid, phenelzine, tranylcypromine, selegiline and moclobemide.

In still another representative embodiment, HAT-modulating compounds that decrease the level and/or activity of a HAT may be used to reduce flushing side effects of chemotherapeutic agents, such as cyclophosphamide, tamoxifen.

In another embodiment, HAT-modulating compounds that decrease the level and/or activity of a HAT may be used to reduce flushing side effects of calcium channel blockers, such as amlodipine.

In another embodiment, HAT-modulating compounds that decrease the level and/or activity of a HAT may be used to reduce flushing side effects of antibiotics. For example, HAT-modulating compounds that decrease the level and/or activity of a HAT can be used in combination with levofloxacin. Levofloxacin is used to treat infections of the sinuses, skin, lungs, ears, airways, bones, and joints caused by susceptible bacteria. Levofloxacin also is frequently used to treat urinary infections, including those resistant to other antibiotics, as well as prostatitis. Levofloxacin is effective in treating infectious diarrheas caused by E. coli, Campylobacter jejuni, and shigella bacteria. Levofloxacin also can be used to treat various obstetric infections, including mastitis. Ocular Disorders One aspect of the present invention is a method for inhibiting, reducing or otherwise treating vision impairment by administering to a patient a therapeutic dosage of HAT modulator selected from a compound disclosed herein, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

Glaucoma describes a group of disorders which are associated with a visual field defect, cupping of the optic disc, and optic nerve damage. These are commonly referred to as glaucomatous optic neuropathies. Most glaucomas are usually, but not always, associated with a rise in intraocular pressure. Exemplary forms of glaucoma include Glaucoma and Penetrating Keratoplasty, Acute Angle Closure, Chronic Angle Closure, Chronic Open Angle, Angle Recession, Aphakic and Pseudophakic, Drug-Induced, Hyphema, Intraocular Tumors, Juvenile, Lens-Particle, Low Tension, Malignant, Neovascular, Phacolytic, Phacomorphic, Pigmentary, Plateau Iris, Primary Congenital, Primary Open Angle, Pseudoexfoliation, Secondary Congenital, Adult Suspect, Unilateral, Uveitic, Ocular Hypertension, Ocular Hypotony, Posner-Schlossman Syndrome and Scleral Expansion Procedure in Ocular Hypertension & Primary Open-angle Glaucoma.

Intraocular pressure can also be increased by various surgical procedures, such as phacoemulsification (i.e., cataract surgery) and implanation of structures such as an artificial lens. In addition, spinal surgeries in particular, or any surgery in which the patient is prone for an extended period of time can lead to increased interoccular pressure.

Optic neuritis (ON) is inflammation of the optic nerve and causes acute loss of vision. It is highly associated with multiple sclerosis (MS) as 15-25% of MS patients initially present with ON, and 50-75% of ON patients are diagnosed with MS. ON is also associated with infection (e.g., viral infection, meningitis, syphilis), inflammation (e.g., from a vaccine), infiltration and ischemia.

Another condition leading to optic nerve damage is anterior ischemic optic neuropathy (AION). There are two types of AION. Arteritic AION is due to giant cell arteritis (vasculitis) and leads to acute vision loss. Non-arteritic AION encompasses all cases of ischemic optic neuropathy other than those due to giant cell arteritis. The pathophysiology of AION is unclear although it appears to incorporate both inflammatory and ischemic mechanisms.

Other damage to the optic nerve is typically associated with demyleination, inflammation, ischemia, toxins, or trauma to the optic nerve. Exemplary conditions where the optic nerve is damaged include Demyelinating Optic Neuropathy (Optic Neuritis, Retrobulbar Optic Neuritis), Optic Nerve Sheath Meningioma, Adult Optic Neuritis, Childhood Optic Neuritis, Anterior Ischemic Optic Neuropathy, Posterior Ischemic Optic Neuropathy, Compressive Optic Neuropathy, Papilledema, Pseudopapilledema and Toxic/Nutritional Optic Neuropathy.

Other neurological conditions associated with vision loss, albeit not directly associated with damage to the optic nerve, include Amblyopia, Bells Palsy, Chronic Progressive External Opthalmoplegia, Multiple Sclerosis, Pseudotumor Cerebri and Trigeminal Neuralgia.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disruption of the macula (e.g., exudative or non-exudative macular degeneration).

Exemplary retinal diseases include Exudative Age Related Macular Degeneration, Nonexudative Age Related. Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis; Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis. One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a HAT modulator disclosed herein.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a HAT modulator disclosed herein. Ocular surgeries include cataract, iridotomy and lens replacements. Another aspect of the invention is the treatment, including inhibition and prophylactic treatment, of age related ocular diseases include cataracts, dry eye, retinal damage and the like, by administering to the subject in need of such treatment a therapeutic dosage of a HAT modulator disclosed herein.

The formation of cataracts is associated with several biochemical changes in the lens of the eye, such as decreased levels of antioxidants ascorbic acid and glutathione, increased lipid, amino acid and protein oxidation, increased sodium and calcium, loss of amino acids and decreased lens metabolism. The lens, which lacks blood vessels, is suspended in extracellular fluids in the anterior part of the eye. Nutrients, such as ascorbic acid, glutathione, vitamin E, selenium, bioflavonoids and carotenoids are required to maintain the transparency of the lens. Low levels of selenium results in an increase of free radical-inducing hydrogen peroxide, which is neutralized by the selenium-dependent antioxidant enzyme glutathione peroxidase. Lens-protective glutathione peroxidase is also dependent on the amino acids methionine, cysteine, glycine and glutamic acid.

Cataracts can also develop due to an inability to properly metabolize galactose found in dairy products that contain lactose, a disaccharide composed of the monosaccharide galactose and glucose. Cataracts can be prevented, delayed, slowed and possibly even reversed if detected early and metabolically corrected. Retinal damage is attributed, inter alia, to free radical initiated reactions in glaucoma, diabetic retinopathy and age-related macular degeneration (AMD). The eye is a part of the central nervous system and has limited regenerative capability. The retina is composed of numerous nerve cells which contain the highest concentration of polyunsaturated fatty acids (PFA) and subject to oxidation. Free radicals are generated by UV light entering the eye and mitochondria in the rods and cones, which generate the energy necessary to transform light into visual impulses. Free radicals cause peroxidation of the PFA by hydroxyl or superoxide radicals which in turn propagate additional free radicals. The free radicals cause temporary or permanent damage to retinal tissue. Glaucoma is usually viewed as a disorder that causes an elevated intraocular pressure (IOP) that results in permanent damage to the retinal nerve fibers, but a sixth of all glaucoma cases do not develop an elevated IOP. This disorder is now perceived as one of reduced vascular perfusion and an increase in neurotoxic factors. Recent studies have implicated elevated levels of glutamate, nitric oxide and peroxynitirite in the eye as the causes of the death of retinal ganglion cells. Neuroprotective agents may be the future of glaucoma care. For example, nitric oxide synthase inhibitors block the formation of peroxynitrite from nitric oxide and superoxide. In a recent study, animals treated with aminoguanidine, a nitric oxide synthase inhibitor, had a reduction in the loss of retinal ganglion cells. It was concluded that nitric oxide in the eye caused cytotoxicity in many tissues and neurotoxicity in the central nervous system.

Diabetic retinopathy occurs when the underlying blood vessels develop microvascular abnormalities consisting primarily of microaneurysms and intraretinal hemorrhages. Oxidative metabolites are directly involved with the pathogenesis of diabetic retinopathy and free radicals augment the generation of growth factors that lead to enhanced proliferative activity. Nitric oxide produced by endothelial cells of the vessels may also cause smooth muscle cells to relax and result in vasodilation of segments of the vessel. Ischemia and hypoxia of the retina occur after thickening of the arterial basement membrane, endothelial proliferation and loss of pericytes. The inadequate oxygenation causes capillary obliteration or nonperfusion, arteriolar-venular shunts, sluggish blood flow and an impaired ability of RBCs to release oxygen. Lipid peroxidation of the retinal tissues also occurs as a result of free radical damage.

The macula is responsible for our acute central vision and composed of light-sensing cells (cones) while the underlying retinal pigment epithelium (RPE) and choroid nourish and help remove waste materials. The RPE nourishes the cones with the vitamin A substrate for the photosensitive pigments and digests the cones shed outer tips. RPE is exposed to high levels of UV radiation, and secretes factors that inhibit angiogenesis. The choroid contains a dense vascular network that provides nutrients and removes the waste materials. In AMD, the shed cone tips become indigestible by the RPE, where the cells swell and die after collecting too much undigested material. Collections of undigested waste material, called drusen, form under the RPE. Photoxic damage also causes the accumulation of lipofuscin in RPE cells. The intracellular lipofuscin and accumulation of drusen in Bruch's membrane interferes with the transport of oxygen and nutrients to the retinal tissues, and ultimately leads to RPE and photoreceptor dysfunction. In exudative AMD, blood vessels grow from the choriocapillaris through defects in Bruch's membrane and may grow under the RPE, detaching it from the choroid, and leaking fluid or bleeding.

Macular pigment, one of the protective factors that prevent sunlight from damaging the retina, is formed by the accumulation of nutritionally derived carotenoids, such as lutein, the fatty yellow pigment that serves as a delivery vehicle for other important nutrients and zeaxanthin. Antioxidants such as vitamins C and E, beta-carotene and lutein, as well as zinc, selenium and copper, are all found in the healthy macula. In addition to providing nourishment, these antioxidants protect against free radical damage that initiates macular degeneration.

Another aspect of the invention is the prevention or treatment of damage to the eye caused by stress, chemical insult or radiation, by administering to the subject in need of such treatment a therapeutic dosage of a HAT modulator disclosed herein. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include one or more HAT inhibitors and one or more therapeutic agents for the treatment of an ocular disorder. For example, one or, more HAT-inhibiting compounds can be combined with an effective amount of one or more of: an agent that reduces intraocular pressure, an agent for treating glaucoma, an agent for treating optic neuritis, an agent for treating CMV Retinopathy, an agent for treating multiple sclerosis, and/or an antibiotic, etc.

In one embodiment, a HAT modulator can be administered in conjunction with a therapy for reducing intraocular pressure. One group of therapies involves blocking aqueous production. For example, topical beta-adrenergic antagonists (timolol and betaxolol) decrease aqueous production. Topical timolol causes IOP to fall in 30 minutes with peak effects in 1-2 hours. A reasonable regimen is Timoptic 0.5%, one drop every 30 minutes for 2 doses. The carbonic anhydrase inhibitor, acetazolamide, also decreases aqueous production and should be given in conjunction with topical beta-antagonists. An initial dose of 500 mg is administered followed by 250 mg every 6 hours. This medication may be given orally, intramuscularly, or intravenously. In addition, alpha 2-agonists (e.g., Apraclonidine) act by decreasing aqueous production. Their effects are additive to topically administered beta-blockers. They have been approved for use in controlling an acute rise in pressure following anterior chamber laser procedures, but has been reported effective in treating acute closed-angle glaucoma. A reasonable regimen is 1 drop every 30 minutes for 2 doses.

A second group of therapies for reducing intraocular pressure involve reducing vitreous volume. Hyperosmotic agents can be used to treat an acute attack. These agents draw water out of the globe by making the blood hyperosmolar. Oral glycerol in a dose of 1 mL/kg in a cold 50% solution (mixed with lemon juice to make it more palatable) often is used. Glycerol is converted to glucose in the liver; persons with diabetes may need additional insulin if they become hyperglycemic after receiving glycerol. Oral isosorbide is a metabolically inert alcohol that also can be used as an osmotic agent for patients with acute angle-closure glaucoma. Usual dose is 100 g taken p.o. (220 cc of a 45% solution). This inert alcohol should not be confused with isosorbide dinitrate, a nitrate-based cardiac medication used for angina and for congestive heart failure. Intravenous mannitol in a dose of 1.0-1.5 mg/kg also is effective and is well tolerated in patients with nausea and vomiting. These hyperosmotic agents should be used with caution in any patient with a history of congestive heart failure.

A third group of therapies involve facilitating aqueous outflow from the eye. Miotic agents pull the iris from the iridocorneal angle and may help to relieve the obstruction of the trabecular meshwork by the peripheral iris. Pilocarpine 2% (blue eyes)-4% (brown eyes) can be administered every 15 minutes for the first 1-2 hours. More frequent administration or higher doses may precipitate a systemic cholinergic crisis. NSAIDS are sometimes used to reduce inflammation. Exemplary therapeutic agents for reducing intraocular pressure include ALPHAGAN® P (Allergan) (brimonidine tartrate ophthalmic solution), AZOPT® (Alcon) (brinzolamide ophthalmic suspension), BETAGAN® (Allergan) (levobunolol hydrochloride ophthalmic solution, USP), BETIMOL® (Vistakon) (timolol ophthalmic solution), BETOPTIC S® (Alcon) (betaxolol HCl), BRIMONIDINE TARTRATE (Bausch & Lomb), CARTEOLOL HYDROCHLORIDE (Bausch & Lomb), COSOPT® (Merck) (dorzolamide hydrochloride-timolol maleate ophthalmic solution), LUMIGAN® (Allergan) (bimatoprost ophthalmic solution), OPTIPRANOLOL® (Bausch & Lomb) (metipranolol ophthalmic solution), TIMOLOL GFS (Falcon) (timolol maleate ophthalmic gel forming solution), TIMOPTIC® (Merck) (timolol maleate ophthalmic solution), TRA V AT AN® (Alcon) (travoprost ophthalmic solution), TRUS OPT® (Merck) (dorzolamide hydrochloride ophthalmic solution) and XALATAN® (Pharmacia & Upjohn) (latanoprost ophthalmic solution).

In one embodiment, a HAT modulator can be administered in conjunction with a therapy for treating and/or preventing glaucoma. An example of a glaucoma drug is DARANIDE® Tablets (Merck) (Dichlorphenamide).

In one embodiment, a HAT modulator can be administered in conjunction with a therapy for treating and/or preventing optic neuritis. Examples of drugs for optic neuritis include DECADRON® Phosphate Injection (Merck) (Dexamethasone Sodium Phosphate), DEPO-MEDROL® (Pharmacia & Upjohn) (methylprednisolone acetate), HYDROCORTONE® Tablets (Merck) (Hydrocortisone), ORAPRED® (Biomarin) (prednisolone sodium phosphate oral solution) and PEDIAPRED® (Celltech) (prednisolone sodium phosphate, USP).

In one embodiment, a HAT modulator can be administered in conjunction with a therapy for treating and/or preventing CMV Retinopathy. Treatments for CMV retinopathy include CYTO VENE® (ganciclovir capsules) and VALCYTE® (Roche Laboratories) (valganciclovir hydrochloride tablets).

In one embodiment, a HAT modulator can be administered in conjunction with a therapy for treating and/or preventing multiple sclerosis. Examples of such drugs include DANTRIUM® (Procter & Gamble Pharmaceuticals) (dantrolene sodium), NOVANTRONE® (Serono) (mitoxantrone), AVONEX® (Biogen Idee) (Interferon beta-1a), BETASERON® (Berlex) (Interferon beta-1b), COPAXONE® (Teva Neuroscience) (glatiramer acetate injection) and REB IF® (Pfizer) (interferon beta-Ia). In addition, macrolide and/or mycophenolic acid, which has multiple activities, can be co-administered with a HAT modulator. Macrolide antibiotics include tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide.

Mitochondrial-Associated Diseases and Disorders

In certain embodiments, the invention provides methods for treating diseases or disorders that would benefit from increased mitochondrial activity. The methods involve administering to a subject in need thereof a therapeutically-effective amount of a HAT inhibiting compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analysis are summarized in Cohen and Gold, Cleveland Clinic Journal of Medicine, 68: 625-642 (2001). One method for diagnosing a mitochondrial dysfunction is the Thor-Byrne-ier scale (see e.g., Cohen and Gold, supra; Collin S. et al., Eur Neurol. 36: 260-267 (1996)). Other methods for determining mitochondrial number and function include, for example, enzymatic assays (e.g., a mitochondrial enzyme or an ATP biosynthesis factor such as an ETC enzyme or a Krebs cycle enzyme), determination or mitochondrial mass, mitochondrial volume, and/or mitochondrial number, quantification of mitochondrial DNA, monitoring intracellular calcium homeostasis and/or cellular responses to perturbations of this homeostasis, evaluation of response to an apoptogenic stimulus, determination of free radical production. Such methods are known in the art and are described, for example, in U.S. Patent Publication No. 2002/0049176 and references cited therein.

Mitochondria are critical for the survival and proper function of almost all types of eukaryotic cells. Mitochondria in virtually any cell type can have congenital or acquired defects that affect their function. Thus, the clinically significant signs and symptoms of mitochondrial defects affecting respiratory chain function are heterogeneous and variable depending on the distribution of defective mitochondria among cells and the severity of their deficits, and upon physiological demands upon the affected cells. Nondividing tissues with high energy requirements, e.g. nervous tissue, skeletal muscle and cardiac muscle are particularly susceptible to mitochondrial respiratory chain dysfunction, but any organ system can be affected. Diseases and disorders associated with mitochondrial dysfunction include diseases and disorders in which deficits in mitochondrial respiratory chain activity contribute to the development of pathophysiology of such diseases or disorders in a mammal. This includes 1) congenital genetic deficiencies in activity of one or more components of the mitochondrial respiratory chain; and 2) acquired deficiencies in the activity of one or more components of the mitochondrial respiratory chain, wherein such deficiencies are caused by a) oxidative damage during aging; b) elevated intracellular calcium; c) exposure of affected cells to nitric oxide; d) hypoxia or ischemia; e) microtubule-associated deficits in axonal transport of mitochondria, or f) expression of mitochondrial uncoupling proteins. Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis. Exemplary diseases or disorders that would benefit from increased mitochondrial activity include, for example, AD (Alzheimer's Disease), ADPD (Alzheimer's Disease and Parkinsons's Disease), AMDF (Ataxia, Myoclonus and Deafness), auto-immune disease, cancer, CIPO (Chronic Intestinal Pseudoobstruction with myopathy and Opthalmoplegia), congenital muscular dystrophy, CPEO (Chronic Progressive External Opthalmoplegia), DEAF (Maternally inherited DEAFness or aminoglycoside-induced DEAFness). (Dementia and Chorea), diabetes mellitus (Type I or Type II), DIDMOAD (Diabetes Insipidus, Diabetes Mellitus, Optic Atrophy, Deafness), DMDF (Diabetes Mellitus and Deafness), dystonia, Exercise Intolerance, ESOC (Epilepsy, Strokes, Optic atrophy, and Cognitive decline), FBSN (Familial Bilateral Striatal Necrosis), FICP (Fatal Infantile 5 Cardiomyopathy Plus, a MELAS-associated cardiomyopathy), GER (Gastrointestinal Reflux), HD (Huntington's Disease), KSS (Kearns Sayre Syndrome), “later-onset” myopathy, LDYT (Leber's hereditary optic neuropathy and DYsTonia). Leigh's Syndrome, LHON (Leber Hereditary Optic Neuropathy), LIMM (Lethal Infantile Mitochondrial Myopathy), MDM (Myopathy and Diabetes Mellitus), MELAS (Mitochondrial Encephalomyopathy, Lactic Acidosis, and Stroke-like episodes), MEPR (Myoclonic Epilepsy and Psychomotor Regression), MERME (MERRF/MELAS overlap disease), MERRF (Myoclonic Epilepsy and Ragged Red Muscle Fibers), MHCM (Maternally Inherited Hypertrophic CardioMyopathy), MICM (Maternally Inherited Cardiomyopathy), MILS (Maternally Inherited Leigh Syndrome), Mitochondrial Encephalo cardiomyopathy, Mitochondrial Encephalomyopathy, MM (Mitochondrial Myopathy), MMC (Maternal Myopathy and Cardiomyopathy), MNGIE (Myopathy and external opthalmoplegia, Neuropathy, Gastro-Intestinal, Encephalopathy), Multisystem Mitochondrial Disorder (myopathy, encephalopathy, blindness, hearing loss, peripheral neuropathy), NARP (Neurogenic muscle weakness, Ataxia, and Retinitis Pigmentosa; alternate phenotype at this locus is reported as Leigh Disease), PD (Parkinson's Disease), Pearson's Syndrome, PEM (Progressive Encephalopathy), PEO (Progressive External Opthalmoplegia), PME (Progressive Myoclonus Epilepsy), PMPS (Pearson Marrow-Pancreas Syndrome), psoriasis, RTT (Rett Syndrome), schizophrenia, SIDS (Sudden Infant Death Syndrome), SNHL (Sensorineural Hearing Loss), Varied Familial Presentation (clinical manifestations range from spastic paraparesis to multisystem progressive disorder & fatal cardiomyopathy to truncal ataxia, dysarthria, severe hearing loss, mental regression, ptosis, opthalmoparesis, distal cyclones, and diabetes mellitus), or Wolfram syndrome. Other diseases and disorders that would benefit from increased mitochondrial activity include, for example, Friedreich's ataxia and other ataxias, amyotrophic lateral sclerosis (ALS) and other motor neuron diseases, macular degeneration, epilepsy, Alpers syndrome, Multiple mitochondrial DNA deletion syndrome, MtDNA depletion syndrome, Complex I deficiency, Complex II (SDH) deficiency, Complex III deficiency, Cytochrome c oxidase (COX, Complex IV) deficiency, Complex deficiency, Adenine Nucleotide Translocator (ANT) deficiency, Pyruvate dehydrogenase (PDH) deficiency, Ethylmalonic aciduria with lactic acidemia, 3-Methyl glutaconic aciduria with lactic acidemia, Refractory epilepsy with declines during infection, Asperger syndrome with declines during infection, Autism with declines during infection, Attention deficit hyperactivity disorder (ADHD), Cerebral palsy with declines during infection, Dyslexia with declines during infection, materially inherited thrombocytopenia and leukemia syndrome, MARIAHS syndrome (Mitochondrial ataxia, recurrent infections, aphasia, hypouricemia/hypomyelination, seizures, and dicarboxylic aciduria), ND6 dystonia, Cyclic vomiting syndrome with declines during infection, 3-Hydroxy isobutryic aciduria with lactic acidemia, Diabetes mellitus with lactic acidemia, Uridine responsive neurologic syndrome (URNS), Dilated cardiomyopathy, Splenic Lymphoma, and Renal Tubular Acidosis/Diabetes/Ataxis syndrome.

In other embodiments, the invention provides methods for treating a subject suffering from mitochondrial disorders arising from, but not limited to, post-traumatic head injury and cerebral edema, stroke (invention methods useful for preventing or preventing reperfusion injury), Lewy body dementia, hepatorenal syndrome, acute liver failure, NASH (non-alcoholic steatohepatitis), Anti-metastasis/prodifferentiation therapy of cancer, idiopathic congestive heart failure, atrial fibrilation (non-valvular), Wolff-Parkinson-White Syndrome, idiopathic heart block, prevention of reperfusion injury in acute myocardial infarctions, familial migraines, irritable bowel syndrome, secondary prevention of non-Q wave myocardial infarctions, Premenstrual syndrome, Prevention of renal failure in hepatorenal syndrome, anti-phospholipid antibody syndrome, eclampsia/pre-eclampsia, oopause infertility, ischemic heart disease/angina, and Shy-Drager and unclassified dysautonomia syndromes.

In still another embodiment, there are provided methods for the treatment of mitochondrial disorders associated with pharmacological drug-related side effects. Types of pharmaceutical agents that are associated with mitochondrial disorders include reverse transcriptase inhibitors, protease inhibitors, inhibitors of DHOD, and the like. Examples of reverse transcriptase inhibitors include, for example, Azidothymidine (AZT), Stavudine (D4T), Zalcitabine (ddC), Didanosine (DDI), Fluoroiodoarauracil (FIAU), Lamivudine (3TC), Abacavir and the like. Examples of protease inhibitors include, for example, Ritonavir, Indinavir, Saquinavir, Nelfinavir and the like. Examples of inhibitors of dihydroorotate dehydrogenase (DHOD) include, for example, Leflunomide, Brequinar, and the like.

Reverse transcriptase inhibitors not only inhibit reverse transcriptase but also polymerase gamma which is required for mitochondrial function. Inhibition of polymerase gamma activity (e.g., with a reverse transcriptase inhibitor) therefore leads to mitochondrial dysfunction and/or a reduced mitochondrial mass which manifests itself in patients as hyperlactatemia. This type of condition may benefit from an increase in the number of mitochondria and/or an improvement in mitochondrial function, e.g., by administration of a HAT inhibiting compound.

Common symptoms of mitochondrial diseases include cardiomyopathy, muscle weakness and atrophy, developmental delays (involving motor, language, cognitive or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

In certain embodiments, the invention provides methods for treating a disease or disorder that would benefit from increased mitochondrial activity that involves administering to a subject in need thereof one or more HAT inhibiting compounds in combination with another therapeutic agent such as, for example, an agent useful for treating mitochondrial dysfunction (such as antioxidants, vitamins, or respiratory chain cofactors), an agent useful for reducing a symptom associated with a disease or disorder involving mitochondrial dysfunction (such as, an anti-seizure agent, an agent useful for alleviating neuropathic pain, an agent for treating cardiac dysfunction), a cardiovascular agent (as described further below), a chemotherapeutic agent (as described further below), or an anti-neurodegeneration agent (as described further below). In an exemplary embodiment, the invention provides methods for treating a disease or disorder that would benefit from increased mitochondrial activity that involves administering to a subject in need thereof one or more HAT inhibiting compounds in combination with one or more of the following: coenzyme Qi0, L-carnitine, thiamine, riboflavin, niacinamide, folate, vitamin E, selenium, lipoic acid, or prednisone. Compositions comprising such combinations are also provided herein.

In exemplary embodiments, the invention provides methods for treating diseases or disorders that would benefit from increased mitochondrial activity by administering to a subject a therapeutically effective amount of a HAT inhibiting compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migraine, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation.

A gene defect underlying Friedreich's Ataxia (FA), the most common hereditary ataxia, was recently identified and is designated “frataxin”. In FA, after a period of normal development, deficits in coordination develop which progress to paralysis and death, typically between the ages of 30 and 40. The tissues affected most severely are the spinal cord, peripheral nerves, myocardium, and pancreas. Patients typically lose motor control and are confined to wheel chairs, and are commonly afflicted with heart failure and diabetes. The genetic basis for FA involves GAA trinucleotide repeats in an intron region of the gene encoding frataxin. The presence of these repeats results in reduced transcription and expression of the gene. Frataxin is involved in regulation of mitochondrial iron content. When cellular frataxin content is subnormal, excess iron accumulates in mitochondria, promoting oxidative damage and consequent mitochondrial degeneration and dysfunction. When intermediate numbers of GAA repeats are present in the frataxin gene intron, the severe clinical phenotype of ataxia may not develop. However, these intermediate-length trinucleotide extensions are found in 25 to 30% of patients with non-insulin dependent diabetes mellirus, compared to about 5% of the nondiabetic population. In certain embodiments, HAT inhibiting compounds may be used for treating patients with disorders related to deficiencies or defects in frataxin, including Friedreich's Ataxia, myocardial dysfunction, diabetes mellitus and complications of diabetes like peripheral neuropathy.

Muscular dystrophy refers to a family of diseases involving deterioration of neuromuscular structure and function, often resulting in atrophy of skeletal muscle and myocardial dysfunction. In the case of Duchenne muscular dystrophy, mutations or deficits in a specific protein, dystrophin, are implicated in its etiology. Mice with their dystrophin genes inactivated display some characteristics of muscular dystrophy, and have an approximately 50% deficit in mitochondrial respiratory chain activity. A final common pathway for neuromuscular degeneration in most cases is calcium-mediated impairment of mitochondrial function. In certain embodiments, HAT inhibiting compounds may be used for reducing the rate of decline in muscular functional capacities and for improving muscular functional status in patients with muscular dystrophy.

Multiple sclerosis (MS) is a neuromuscular disease characterized by focal inflammatory and autoimmune degeneration of cerebral white matter. Periodic exacerbations or attacks are significantly correlated with upper respiratory tract and other infections, both bacterial and viral, indicating that mitochondrial dysfunction plays a role in MS. Depression of neuronal mitochondrial respiratory chain activity caused by Nitric Oxide (produced by astrocytes and other cells involved in inflammation) is implicated as a molecular mechanism contributing to MS. In certain embodiments, HAT inhibiting compounds maybe used for treatment of patients with multiple sclerosis, both prophylactically and during episodes of disease exacerbation. Epilepsy is often present in patients with mitochondrial cytopathies, involving a range of seizure severity and frequency, e.g. absence, tonic, atonic, myoclonic, and status epilepticus, occurring in isolated episodes or many times daily. In certain embodiments, HAT inhibiting compounds may be used for treating patients with seizures secondary to mitochondrial dysfunction, including reducing frequency and severity of seizure activity.

Metabolic studies on patients with recurrent migraine headaches indicate that deficits in mitochondrial activity are commonly associated with this disorder, manifesting as impaired-oxidative phosphorylation and excess lactate production. Such deficits are not necessarily due to genetic defects in mitochondrial DNA. Migraineurs are hypersensitive to nitric oxide, an endogenous inhibitor of Cytochrome c Oxidase. In addition, patients with mitochondrial cytopathies, e.g. MELAS, often have recurrent migraines. In certain embodiments, HAT inhibiting compounds may be used for treating patients with recurrent migraine headaches, including headaches refractory to ergot compounds or serotonin receptor antagonists. Delays in neurological or neuropsychological development are often found in children with mitochondrial diseases. Development and remodeling of neural connections requires intensive biosynthetic activity, particularly involving synthesis of neuronal membranes and myelin, both of which require pyrimidine nucleotides as cofactors. Uridine nucleotides are involved inactivation and transfer of sugars to glycolipids and glycoproteins. Cytidine nucleotides are derived from uridine nucleotides, and are crucial for synthesis of major membrane phospholipid constituents like phosphatidylcholine, which receives its choline moiety from cytidine diphosphocholine. In the case of mitochondrial dysfunction (due to either mitochondrial DNA defects or any of the acquired or conditional deficits like exicitoxic or nitric oxide-mediated mitochondrial dysfunction) or other conditions resulting in impaired pyrimidine synthesis, cell proliferation and axonal extension is impaired at crucial stages in development of neuronal interconnections and circuits, resulting in delayed or arrested development of neuropsychological functions like language, motor, social, executive function, and cognitive skills. In autism for example, magnetic resonance spectroscopy measurements of cerebral phosphate compounds indicates that there is global undersynthesis of membranes and membrane precursors indicated by reduced levels of uridine diphospho-sugars, and cytidine nucleotide derivatives involved in membrane synthesis. Disorders characterized by developmental delay include Rett's Syndrome, pervasive developmental delay (or PDD-NOS “pervasive developmental delay not otherwise specified” to distinguish it from specific subcategories like autism), autism, Asperger's Syndrome, and Attention Deficit/Hyperactivity Disorder (ADHD), which is becoming recognized as a delay or lag in development of neural circuitry underlying executive functions. In certain embodiments, HAT inhibiting compounds may be useful for treating treating patients with neurodevelopmental delays (e.g., involving motor, language, executive function, and cognitive skills), or other delays or arrests of neurological and neuropsychological development in the nervous system and somatic development in non-neural tissues like muscle and endocrine glands. The two most significant severe neurodegenerative diseases associated with aging, Alzheimer's Disease (AD) and Parkinson's Disease (PD), both involve mitochondrial dysfunction in their pathogenesis. Complex I deficiencies in particular are frequently found not only in the nigrostriatal neurons that degenerate in Parkinson's disease, but also in peripheral tissues and cells like muscle and platelets of Parkinson's Disease patients. In Alzheimer's Disease, mitochondrial respiratory chain activity is often depressed, especially Complex IV (Cytochrome c Oxidase). Moreover, mitochondrial respiratory function altogether is depressed as a consequence of aging, further amplifying the deleterious sequelae of additional molecular lesions affecting respiratory chain function. Other factors in addition to primary mitochondrial dysfunction underlie neurodegeneration in AD, PD, and related disorders. Excitotoxic stimulation and nitric oxide are implicated in both diseases, factors which both exacerbate mitochondrial respiratory chain deficits and whose deleterious actions are exaggerated on a background of respiratory chain dysfunction. Huntington's Disease also involves mitochondrial dysfunction in affected brain regions, with cooperative interactions of excitotoxic stimulation and mitochondrial dysfunction contributing to neuronal degeneration. In certain embodiments, HAT inhibiting compounds may be useful for treating and attenuating progression of age-related neurodegenerative diseases including AD and PD. One of the major genetic defects in patients with Amyotrophic Lateral Sclerosis (ALS or Lou Gehrig's Disease) is mutation or deficiency in Copper-Zinc Superoxide Dismutase (SOD 1), an antioxidant enzyme. Mitochondria both produce and are primary targets for reactive oxygen species. Inefficient transfer of electrons to oxygen in mitochondria is the most significant physiological source of free radicals in mammalian systems. Deficiencies in antioxidants or antioxidant enzymes can result in or exacerbate mitochondrial degeneration. Mice transgenic for mutated SOD1 develop symptoms and pathology similar to those in human ALS. The development of the disease in these animals has been shown to involve oxidative destruction of mitochondria followed by functional decline of motor neurons and onset of clinical symptoms. Skeletal muscle from ALS patients has low mitochondrial Complex I activity. In certain embodiments, HAT inhibiting compounds may be useful for treating ALS, for reversing or slowing the progression of clinical symptoms.

Oxygen deficiency results in both direct inhibition of mitochondrial respiratory chain activity by depriving cells of a terminal electron acceptor for Cytochrome c reoxidation at Complex IV, and indirectly, especially in the nervous system, via secondary post-anoxic excitotoxicity and nitric oxide formation. In conditions like cerebral anoxia, angina or sickle cell anemia crises, tissues are relatively hypoxic. In such cases, compounds that increase mitochondrial activity provide protection of affected tissues from deleterious effects of hypoxia, attenuate secondary delayed cell death, and accelerate recovery from hypoxic tissue stress and injury. In certain embodiments, HAT inhibiting compounds may be useful for preventing delayed cell death (apoptosis in regions like the hippocampus or cortex occurring about 2 to 5 days after an episode of cerebral ischemia) after ischemic or hypoxic insult to the brain.

Acidosis due to renal dysfunction is often observed in patients with mitochondrial disease, whether the underlying respiratory chain dysfunction is congenital or induced by ischemia or cytotoxic agents like cisplatin. Renal tubular acidosis often requires administration of exogenous sodium bicarbonate to maintain blood and tissue pH. In certain embodiments, HAT inhibiting compounds may be useful for treating renal tubular acidosis and other forms of renal dysfunction caused by mitochondrial respiratory chain deficits.

During normal aging, there is a progressive decline in mitochondrial respiratory chain function. Beginning about age 40, there is an exponential rise in accumulation of mitochondrial DNA defects in humans, and a concurrent decline in nuclear-regulated elements of mitochondrial respiratory activity. Many mitochondrial DNA lesions have a selection advantage during mitochondrial turnover, especially in postmitotic cells. The proposed mechanism is that mitochondria with a defective respiratory chain produce less oxidative damage to themselves than do mitochondria with intact functional respiratory chains (mitochondrial respiration is the primary source of free radicals in the body). Therefore, normally-functioning mitochondria accumulate oxidative damage to membrane lipids more rapidly than do defective mitochondria, and are therefore “tagged” for degradation by lysosomes. Since mitochondria within cells have a half life of about 10 days, a selection advantage can result in rapid replacement of functional mitochondria with those with diminished respiratory activity, especially in slowly dividing cells. The net result is that once a mutation in a gene for a mitochondrial protein that reduces oxidative damage to mitochondria occurs, such defective mitochondria will rapidly populate the cell, diminishing or eliminating its respiratory capabilities. The accumulation of such cells results in aging or degenerative disease at the organismal level. This is consistent with the progressive mosaic appearance of cells with defective electron transport activity in muscle, with cells almost devoid of Cytochrome c Oxidase (COX) activity interspersed randomly amidst cells with normal activity, and a higher incidence of COX-negative cells in biopsies from older subjects. The organism, during aging, or in a variety of mitochondrial diseases, is thus faced with a situation in which irreplaceable postmitotic cells (e.g. neurons, skeletal and cardiac muscle) must be preserved and their function maintained to a significant degree, in the face of an inexorable progressive decline in mitochondrial respiratory chain function. Neurons with dysfunctional mitochondria become progressively more sensitive to insults like excitotoxic injury. Mitochondrial failure contributes to most degenerative diseases (especially neurodegeneration) that accompany aging. Congenital mitochondrial diseases often involve early-onset neurodegeneration similar in fundamental mechanism to disorders that occur during aging of people born with no[pi]nal mitochondria. In certain embodiments, HAT inhibiting compounds may be useful for treating or attenuating cognitive decline and other degenerative consequences of aging.

Mitochondrial DNA damage is more extensive and persists longer than nuclear DNA damage in cells subjected to oxidative stress or cancer chemotherapy agents like cisplatin due to both greater vulnerability and less efficient repair of mitochondrial DNA. Although mitochondrial DNA may be more sensitive to damage than nuclear DNA, it is relatively resistant, in some situations, to mutagenesis by chemical carcinogens. This is because mitochondria respond to some types of mitochondrial DNA damage by destroying their defective genomes rather than attempting to repair them. This results in global mitochondrial dysfunction for a period after cytotoxic chemotherapy. Clinical use of chemotherapy agents like cisplatin, mitomycin, and Cytoxan is often accompanied by debilitating “chemotherapy fatigue”, prolonged periods of weakness and exercise intolerance which may persist even after recovery from hematologic and gastrointestinal toxicities of such agents. In certain embodiments, HAT inhibiting compounds may be useful for treatment and prevention of side effects of cancer chemotherapy related to mitochondrial dysfunction.

A crucial function of the ovary is to maintain integrity of the mitochondrial genome in oocytes, since mitochondria passed onto a fetus are all derived from those present in oocytes at the time of conception. Deletions in mitochondrial DNA become detectable around the age of menopause, and are also associated with abnormal menstrual cycles. Since cells cannot directly detect and respond to defects in mitochondrial DNA, but can only detect secondary effects that affect the cytoplasm, like impaired respiration, redox status, or deficits in pyrimidine synthesis, such products of mitochondrial function participate as a signal for oocyte selection and follicular atresia, ultimately triggering menopause when maintenance of mitochondrial genomic fidelity and functional activity can no longer be guaranteed. This is analogous to apoptosis in cells with DNA damage, which undergo an active process of cellular suicide when genomic fidelity can no longer be achieved by repair processes. Women with mitochondrial cytopathies affecting the gonads often undergo premature menopause or display primary cycling abnormalities. Cytotoxic cancer chemotherapy often induces premature menopause, with a consequent increased risk of osteoporosis. Chemotherapy-induced amenorrhea is generally due to primary ovarian failure. The incidence of chemotherapy-induced amenorrhea increases as a function of age in premenopausal women receiving chemotherapy, pointing toward mitochondrial involvement. Inhibitors of mitochondrial respiration or protein synthesis inhibit hormone-induced ovulation, and furthermore inhibit production of ovarian steroid hormones in response to pituitary gonadotropins. Women with Down's syndrome typically undergo menopause prematurely, and also are subject to early onset of Alzheimer-like dementia. Low activity of cytochrome oxidase is consistently found in tissues of Down's patients and in late-onset Alzheimer's Disease. Appropriate support of mitochondrial function or compensation for mitochondrial dysfunction therefore is useful for protecting against age-related or chemotherapy-induced menopause or irregularities of menstrual cycling or ovulation. In certain embodiments, HAT inhibiting compounds maybe useful for treating and preventing amenorrhea, irregular ovulation, menopause, or secondary consequences of menopause.

In certain embodiments, HAT inhibiting compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external opthalmoplegia, the Kearns-Sayre syndrome (with opthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal). Muscle biopsy specimens stained with modified Gomori's trichrome stain show ragged red fibers due to excessive accumulation of mitochondria. Biochemical defects in substrate transport and utilization, the Krebs cycle, oxidative phosphorylation, or the respiratory chain are detectable. Numerous mitochondrial DNA point mutations and deletions have been described, transmitted in a maternal, nonmendelian inheritance pattern. Mutations in nuclear-encoded mitochondrial enzymes occur. In certain embodiments, HAT inhibiting compounds may be useful for treating patients suffering from toxic damage to mitochondria, such as, toxic damage due to, calcium accumulation, excitotoxicity, nitric oxide exposure, drug induced toxic damage, or hypoxia.

A fundamental mechanism of cell injury, especially in excitable tissues, involves excessive calcium entry into cells, as a result of either leakage through the plasma membrane or defects in intracellular calcium handling mechanisms.

Mitochondria are major sites of calcium sequestration, and preferentially utilize energy from the respiratory chain for taking up calcium rather than for ATP synthesis, which results in a downward spiral of mitochondrial failure, since calcium uptake into mitochondria results in diminished capabilities for energy transduction. Excessive stimulation of neurons with excitatory amino acids is a common mechanism of cell death or injury in the central nervous system. Activation of glutamate receptors, especially of the subtype designated NMDA receptors, results in mitochondrial dysfunction, in part through elevation of intracellular calcium during excitotoxic stimulation. Conversely, deficits in mitochondrial respiration and oxidative phosphorylation sensitizes cells to excitotoxic stimuli, resulting in cell death or injury during exposure to levels of excitotoxic neurotransmitters or toxins that would be innocuous to normal cells.

Nitric oxide (about 1 micromolar) inhibits cytochrome oxidase (Complex IV) and thereby inhibits mitochondrial respiration; moreover, prolonged exposure to nitric oxide (NO) irreversibly reduces Complex I activity. Physiological or pathophysiological concentrations of NO thereby inhibit pyrimidine biosynthesis. Nitric oxide is implicated in a variety of neurodegenerative disorders including inflammatory and autoimmune diseases of the central nervous system, and is involved in mediation of excitotoxic and post-hypoxic damage to neurons. Oxygen is the terminal electron acceptor in the respiratory chain. Oxygen deficiency impairs electron transport chain activity, resulting in diminished pyrimidine synthesis as well as diminished ATP synthesis via oxidative phosphorylation. Human cells proliferate and retain viability under virtually anaerobic conditions if provided with uridine and pyruvate (or a similarly effective agent for oxidizing NADH to optimize glycolytic ATP production).

In certain embodiments, HAT inhibiting compounds may be useful for treating diseases or disorders associated with mitochondrial deregulation. Transcription of mitochondrial DNA encoding respiratory chain components requires nuclear factors. In neuronal axons, mitochondria must shuttle back and forth to the nucleus in order to maintain respiratory chain activity. If axonal transport is impaired by hypoxia or by drugs like taxol which affect microtubule stability, mitochondria distant from the nucleus undergo loss of cytochrome oxidase activity. Accordingly, treatment with a HAT inhibiting compound may be useful for promoting nuclear-mitochondrial interactions. Mitochondria are the primary source of free radicals and reactive oxygen species, due to spillover from the mitochondrial respiratory chain, especially when defects in one or more respiratory chain components impairs orderly transfer of electrons from metabolic intermediates to molecular oxygen. To reduce oxidative damage, cells can compensate by expressing mitochondrial uncoupling proteins (UCP), of which several have been identified. UCP-2 is transcribed in response to oxidative damage, inflammatory cytokines, or excess lipid loads, e.g. fatty liver and steatohepatitis. UCPs reduce spillover of reactive oxygen species from mitochondria by discharging proton gradients across the mitochondrial inner membrane, in effect wasting energy produced by metabolism and rendering cells vulnerable to energy stress as a trade-off for reduced oxidative injury. Muscle Performance

In other embodiments, the invention provides methods for enhancing muscle performance by administering a therapeutically effective amount of a HAT inhibiting compound. For example, HAT inhibiting compounds may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a HAT inhibiting compound that increase mitochondrial activity, increase mitochondrial biogenesis, and/or increase mitochondrial mass.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. An athlete may be hard training, that is, sports activities intensely more than three days a week or for competition. An athlete may also be a fitness enthusiast who seeks to improve general health and well-being, improve energy levels, who works out for about 1-2 hours about 3 times a week.

Enhanced sports performance in manifested by the ability to overcome muscle fatigue, ability to maintain activity for longer periods of time, and have a more effective workout. In the arena of athlete muscle performance, it is desirable to create conditions that permit competition or training at higher levels of resistance for a prolonged period of time. However, acute and intense anaerobic use of skeletal muscles often results in impaired athletic performance, with losses in force and work output, and increased onset of muscle fatigue, soreness, and dysfunction. It is now recognized that even a single exhaustive exercise session, or for that matter any acute trauma to the body such as muscle injury, resistance or exhaustive muscle exercise, or elective surgery, is characterized by perturbed metabolism that affects muscle performance in both short and long term phases. Both muscle metabolic/enzymatic activity and gene expression are affected. For example, disruption of skeletal muscle nitrogen metabolism as well as depletion of sources of metabolic energy occur during extensive muscle activity.

Amino acids, including branched-chain amino acids, are released from muscles followed by their deamination to elevate serum ammonia and local oxidation as muscle fuel sources, which augments metabolic acidosis. In addition, there is a decline in catalytic efficiency of muscle contraction events, as well as an alteration of enzymatic activities of nitrogen and energy metabolism. Further, protein catabolism is initiated where rate of protein synthesis is decreased coupled with an increase in the degradation of non-contractible protein. These metabolic processes are also accompanied by free radical generation which further damages muscle cells.

Recovery from fatigue during acute and extended exercise requires reversal of metabolic and non-metabolic fatiguing factors. Known factors that participate in human muscle fatigue, such as lactate, ammonia, hydrogen ion, etc., provide an incomplete and unsatisfactory explanation of the fatigue/recovery process, and it is likely that additional unknown agents participate (Baker et al., J. Appl. Physiol. 74:2294-2300, 1993; Bazzarre et al., J. Am. Coll. Nutr. 11:505-511, 1992; Dohm et al., Fed. Proc. 44:348-352, 1985; Edwards In: Biochemistry of Exercise, Proceedings of the Fifth & International Symposium on the Biochemistry of Exercise (Kutrgen, Vogel, Poormans, eds.), 1983; MacDougall et al., Acta Physiol. Scand. 146:403-404, 1992; Walser et al., Kidney Int. 32:123-128, 1987). Several studies have also analyzed the effects of nutritional supplements and herbal supplements in enhancing muscle performance. Aside from muscle performance during endurance exercise, free radicals and oxidative stress parameters are affected in pathophysiological states. A substantial body of data now suggests that oxidative stress contributes to muscle wasting or atrophy in pathophysiological states (reviewed in Clarkson, P. M. Antioxidants and physical performance. Grit. Rev. Food Sci. Nutr. 35: 31-41; 1995; Powers, S. K.; Lennon, S. L. Analysis of cellular responses to free radicals: Focus on exercise and skeletal muscle. Proc. Nutr. Soc. 58: 1025-1033; 1999). For example, with respect to muscular disorders where both muscle endurance and function are compensated, the role of nitric oxide (NO), has been implicated. In muscular dystrophies, especially those due to defects in proteins that make up the dystrophin-glycoprotein complex (DGC), the enzyme that synthesizes NO, nitric oxide synthase (NOS), has been associated. Recent studies of dystrophies related to DGC defects suggest that one mechanism of cellular injury is functional ischemia related to alterations in cellular NOS and disruption of a normal protective action of NO. This protective action is the prevention of local ischemia during contraction-induced increases in sympathetic vasoconstriction. Rando (Micros Res Tech 55(4):223-35, 2001), has shown that oxidative injury precedes pathologic changes and that muscle cells with defects in the DGC have an increased susceptibility to oxidant challenges. Excessive lipid peroxidation due to free radicals has also been shown to be a factor in myopathic diseases such as McArdle's disease (Russo et al., Med Hypotheses. 39(2): 147-51, 1992). Furthermore, mitochondrial dysfunction is a well-known correlate of age-related muscle wasting (sarcopenia) and free radical damage has been suggested, though poorly investigated, as a contributing factor (reviewed in Navarro, A.; Lopez-Cepero, J. M.; Sanchez del Pino, M. L. Front. Biosci. 6: D26-44; 2001). Other indications include acute sarcopenia, for example muscle atrophy and/or cachexia associated with burns, bed rest, limb immobilization, or major thoracic, abdominal, and/or orthopedic surgery. It is contemplated that the methods of the present invention will also be effective in the treatment of muscle related pathological conditions.

In certain embodiments, the invention provides novel dietary compositions comprising HAT modulators, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

Other Uses

HAT-modulating compounds that decrease the level and/or activity of a HAT may be used for treating or preventing viral infections (such as infections by influenza, herpes or papilloma virus) or as antifungal agents. In certain embodiments, HAT-modulating compounds that decrease the level and/or activity of a HAT may be administered as part of a combination drug therapy with another therapeutic agent for the treatment of viral diseases, including, for example, acyclovir, ganciclovir and zidovudine, hi another embodiment, HAT-modulating compounds that decrease the level and/or activity of a HAT may be administered as part of a combination drug therapy with another anti-fungal agent including, for example, topical anti-fungals such as ciclopirox, clotrimazole, econazole, miconazole, nystatin, oxiconazole, terconazole, and tomaftate, or systemic anti-fungal such as fluconazole (Diflucan), itraconazole (Sporanox), ketoconazole (Nizoral), and miconazole (Monistat I. V.).

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., —bacterial cells. For example, modulating compounds may be administered to farm animals to improve their ability to withstand farming conditions longer.

HAT-modulating compounds that decrease the level and/or activity of a HAT may also be used to increase lifespan, stress resistance, and resistance to apoptosis in plants. In one embodiment, a compound is applied to plants, e.g., on a periodic basis, or to fungi. In another embodiment, plants are genetically modified to produce a compound. In another embodiment, plants and fruits are treated with a compound prior to picking and shipping to increase resistance to damage during shipping. Plant seeds may also be contacted with compounds described herein, e.g., to preserve them.

In other embodiments, HAT-modulating compounds that decrease the level and/or activity of a HAT may be used for modulating lifespan in yeast cells. Situations in which it may be desirable to extend the lifespan of yeast cells include any process in which yeast is used, e.g., the making of beer, yogurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

HAT-modulating compounds that decrease the level and/or activity of a HAT may also be used to increase lifespan, stress resistance and resistance to apoptosis in insects. In this embodiment, compounds would be applied to useful insects, e.g., bees and other insects that are involved in pollination of plants. In a specific embodiment, a compound would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of HAT-modulating compounds that decrease the level and/or activity of a HAT may also be used as a pesticide by interfering with the regulation of silenced genes and the regulation of apoptosis during development. In this embodiment, a compound may be applied to plants using a method known in the art that ensures the compound is bio-available to insect larvae, and not to plants. HAT-modulating compounds that increase the level and/or activity of a sirtuin protein can be applied to affect the reproduction of organisms such as insects, animals and microorganisms.

### Pharmaceutical Compositions

The HAT-modulating compounds described herein may be formulated in a conventional manner using one or more physiologically acceptable carriers or excipients. For example, sirtuin-modulating compounds and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a HAT-modulating compound may be administered locally, at the site where the target cells are present, i.e., in a specific tissue, organ, or fluid (e.g., blood, cerebrospinal fluid, etc.).

HAT-modulating compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd-oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), HAT-modulating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

HAT-modulating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

HAT-modulating compounds may also be formulated in rectal compositions such as suppositories or retention enemas, e.g., containing conventional suppository bases such as cocoa butter or other glycerides.

In addition to the formulations described previously, HAT-modulating compounds may also be formulated as a depot preparation. Such long acting formulations may be administered by implantation (for example subcutaneously or intramuscularly) or by intramuscular injection. Thus, for example, HAT-modulating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches. In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

One possibility to achieve sustained release kinetics is embedding or encapsulating the active compound into nanoparticles. Nanoparticles can be administrated as powder, as a powder mixture with added excipients or as suspensions. Colloidal suspensions of nanoparticles can easily be administrated through a cannula with small diameter.

Nanoparticles are particles with a diameter from about 5 nm to up to about 1000 nm. The term “nanoparticles” as it is used hereinafter refers to particles formed by a polymeric matrix in which the active compound is dispersed, also known as “nanospheres”, and also refers to nanoparticles which are composed of a core containing the active compound which is surrounded by a polymeric membrane, also known as “nanocapsules”. In certain embodiments, nanoparticles are preferred having a 5 diameter from about 50 nm to about 500 nm, in particular from about 100 nm to about 200 nm.

Nanoparticles can be prepared by in situ polymerization of dispersed monomers or by using preformed polymers. Since polymers prepared in situ are often not biodegradable and/or contain toxicological serious byproducts, nanoparticles from preformed polymers are preferred. Nanoparticles from preformed polymers can be prepared by different techniques, e.g., by emulsion evaporation, solvent displacement, salting-out, mechanical grinding, microprecipitation, and by emulsification diffusion. With the methods described above, nanoparticles can be formed with various types of polymers. For use in the method of the present invention, nanoparticles made from biocompatible polymers are preferred. The term “biocompatible” refers to material that after introduction into a biological environment has no serious effects to the biological environment. From biocompatible polymers those polymers are especially preferred which are also biodegradable. The term “biodegradable” refers to material that after introduction into a biological environment is enzymatically or chemically degraded into smaller molecules, which can be eliminated subsequently.

Examples are polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen and albumin.

Suitable surface modifiers can preferably be selected from known organic and inorganic pharmaceutical excipients. Such excipients include various polymers, low molecular weight oligomers, natural products and surfactants. Preferred surface modifiers include nonionic and ionic surfactants. Representative examples of surface modifiers include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986. Further description on preparing nanoparticles can be found, for example, in U.S. Pat. No. 6,264,922, the contents of which are incorporated herein by reference. Liposomes are a further drug delivery system which is easily injectable.

Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent; for example, a coating of hydrophilic polymer chains such as polyethylene glycol (PEG) to extend the blood circulation lifetime of the liposomes. Such surface-modified liposomes are commonly referred to as “long circulating” or “sterically stabilized” liposomes.

One surface modification to a liposome is the attachment of PEG chains, typically having a molecular weight from about 1000 daltons (Da) to about 5000 Da, and to about 5 mole percent (%) of the lipids making up the liposomes (see, for example, Stealth Liposomes, CRC Press, Lasic, D. and Martin, F., eds., Boca Raton, Fla., (1995)), and the cited references therein. The pharmacokinetics exhibited by such liposomes are characterized by a dose-independent reduction in uptake of liposomes by 5 the liver and spleen via the mononuclear phagocyte system (MPS), and significantly prolonged blood circulation time, as compared to non-surface-modif[iota]ed liposomes, which tend to be rapidly removed from the blood and accumulated in the liver and spleen.

In certain embodiments, the complex is shielded to increase the circulatory half-life of the complex or shielded to increase the resistance of nucleic acid to degradation, for example degradation by nucleases.

As used herein, the term “shielding”, and its cognates such as “shielded”, refers to the ability of “shielding moieties” to reduce the non-specific interaction of the complexes described herein with serum complement or with other species present in serum in vitro or in vivo. Shielding moieties may decrease the complex interaction with or binding to these species through one or more mechanisms, including, for example, non-specific steric or non-specific electronic interactions. Examples of such interactions include non-specific electrostatic interactions, charge interactions, Van der Waals interactions, steric-hindrance and the like. For a moiety to act as a shielding moiety, the mechanism or mechanisms by which it may reduce interaction with, association with or binding to the serum complement or other species does not have to be identified. One can determine whether a moiety can act as a shielding moiety by determining whether or to what extent a complex binds serum species.

It should be noted that “shielding moieties” can be multifunctional. For example, a shielding moiety may also function as, for example, a targeting factor. A shielding moiety may also be referred to as multifunctional with respect to the mechanism(s) by which it shields the complex. While not wishing to be limited by proposed mechanism or theory, examples of such a multifunctional shielding moiety are pH sensitive endosomal membrane-disruptive synthetic polymers, such as PPAA or PEAA. Certain poly(alkyl acrylic acids) have been shown to disrupt endosomal membranes while leaving the outer cell surface membrane intact (Stayton et al. (2000) J. Contrail. Release 65:203-220; Murthy et al. (1999) J. Contrail. Release 61:137-143; WO 99/34831), thereby increasing cellular bioavailability and functioning as a targeting factor. However, PPAA reduces binding of serum complement to complexes in which it is incorporated, thus functioning as a shielding moiety.

Another way to produce a formulation, particularly a solution, of a HAT modulator is through the use of cyclodextrin. By cyclodextrin is meant [alpha]-, [beta]-, or [gamma]-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule. The cyclodextrin of the compositions according to the invention may be [alpha]-, [beta]-, or [gamma]-cyclodextrin. [alpha]-cyclodextrin contains six glucopyranose units; [beta]-cyclodextrin contains seven glucopyranose units; and [gamma]-cyclodextrin contains eight glucopyranose units. The molecule is believed to form a truncated cone having a core opening of 4.7-5.3 angstroms, 6.0-6.5 angstroms, and 7.5-8.3 angstroms in [alpha]-, [beta]-, or [gamma]-cyclodextrin respectively. The composition according to the invention may comprise a mixture of two or more of the [alpha]-, [beta]-, or [gamma]-cyclodextrins. Typically, however, the composition according to the invention will comprise only one of the [alpha]-, [beta]-, or [gamma]-cyclodextrins.

Most preferred cyclodextrins in the compositions according to the invention are amorphous cyclodextrin compounds. By amorphous cyclodextrin is meant non-crystalline mixtures of cyclodextrins wherein the mixture is prepared from [alpha]-, [beta]-, or [gamma]-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl 30 derivatives of [beta]-cyclodextrin, carboxyamidomethyl-[beta]-cyclodextrin, carboxymethyl-[beta]-cyclodextrin, hydroxypropyl-[beta]-cyclodextrin and diethylamino-[beta]-cyclodextrin. One example of resveratrol dissolved in the presence of a cyclodextrin is provided in Marier et al., J. Pharmacol. Exp. Therap. 302:369-373 (2002), the contents of which are incorporated herein by reference, where a 6 mg/mL solution of resveratrol was prepared using 0.9% saline containing 20% hydroxylpropyl-[beta]-cyclodextrin.

As mentioned above, the compositions of matter of the invention comprise an aqueous preparation of preferably substituted amorphous cyclodextrin and one or more HAT modulators. The relative amounts of HAT modulators and cyclodextrin will vary depending upon the relative amount of each of the HAT modulators and the effect of the cyclodextrin on the compound. In general, the ratio of the weight of compound of the HAT modulators to the weight of cyclodextrin compound will be in a range between 1:1 and 1:100. A weight to weight ratio in a range of 1:5 to 1:50 and more preferably in a range of 1:10 to 1:20 of the compound selected from HAT modulators to cyclodextrin are believed to be the most effective for increased circulating availability of the HAT modulator.

Importantly, if the aqueous solution comprising the HAT modulators and a cyclodextrin is to be administered parenterally, especially via the intravenous route, a cyclodextrin will be substantially free of pyrogenic contaminants. Various forms of cyclodextrin, such as forms of amorphous cyclodextrin, maybe purchased from a number of vendors including Sigma-Aldrich, Inc. (St. Louis, Mo., USA). A method for the production of hydroxypropyl-[beta]-cyclodextrin is disclosed in Pitha et al., U.S. Pat. No. 4,727,064 which is incorporated herein by reference. Additional description of the use of cyclodextrin for solubilizing compounds can be found in US 2005/0026849, the contents of which are incorporated herein by reference:

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast melt solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms. See e.g., patents and publications by Cima Labs such as U.S. Pat. Nos. 5,607,697, 5,503,846, 5,223,264, 5,401,513, 5,219,574, and 5 5,178,878, WO 98/46215, WO 98/14179; patents to Fuisz Technologies, now part of Bio Vail, such as U.S. Pat. Nos. 5,871,781, 5,869,098, 5,866,163, 5,851,553, 5,622,719, 5,567,439, and 5,587,172; U.S. Pat. No. 5,464,632 to Prographarm; patents to R. P. Scherer such as U.S. Pat. Nos. 4,642,903, 5,188,825, 5,631,023 and 5,827,541; patents to Yamanouchi-Shaklee such as U.S. Pat. Nos. 5,576,014 and 5,446,464; patents to 10 Janssen such as U.S. Pat. Nos. 5,807,576, 5,635,210, 5,595,761, 5,587,180 and 5,776,491; U.S. Pat. Nos. 5,639,475 and 5,709,886 to Eurand America, Inc.; U.S. Pat. Nos. 5,807,578 and 5,807,577 to L.A.B. Pharmaceutical Research; patents to Schering Corporation such as U.S. Pat. Nos. 5,112,616 and 5,073,374; U.S. Pat. No. 4,616,047 to Laboratoire L. LaFon; U.S. Pat. No. 5,501,861 to Takeda Chemicals Inc., Ltd.; and U.S. Pat. No. 6,316,029 to Elan.

In one example of fast melt tablet preparation, granules for fast melt tablets made by either the spray drying or pre-compacting processes are mixed with excipients and compressed into tablets using conventional tablet making machinery. The granules can be combined with a variety of carriers including low density, high moldability saccharides, low moldability saccharides, polyol combinations, and then directly compressed into a tablet that exhibits an improved dissolution and disintegration profile.

The tablets according to the present invention typically have a hardness of about 2 to about 6 Strong-Cobb units (scu). Tablets within this hardness range disintegrate or dissolve rapidly when chewed. Additionally, the tablets rapidly disintegrate in water. On average, a typical 1.1 to 1.5 gram tablet disintegrates in 1-3 minutes without stirring. This rapid disintegration facilitates delivery of the active material.

The granules used to make the tablets can be, for example, mixtures of low density alkali earth metal salts or carbohydrates. For example, a mixture of alkali earth metal salts includes a combination of calcium carbonate and magnesium hydroxide. Similarly, a fast melt tablet can be prepared according to the methods of the present invention that incorporates the use of A) spray dried extra light calcium carbonate/maltodextrin, B) magnesium hydroxide and C) a eutectic polyol combination including Sorbitol Instant, xylitol and mannitol. These materials have been combined to produce a low density tablet that dissolves very readily and promotes the fast disintegration of the active ingredient. Additionally, the pre-compacted and spray dried granules can be combined in the same tablet.

For fast melt tablet preparation, a HAT modulator useful in the present invention can be in a form such as solid, particulate, granular, crystalline, oily or solution. The HAT modulator for use in the present invention may be a spray dried product or an adsorbate that has been pre-compacted to a harder granular form that reduces the medicament taste. A pharmaceutical active ingredient for use in the present invention may be spray dried with a carrier that prevents the active ingredient from being easily extracted from the tablet when chewed.

In addition to being directly added to the tablets of the present invention, the medicament drug itself can be processed by the pre-compaction process to achieve an increased density prior to being incorporated into the formulation.

The pre-compaction process used in the present invention can be used to deliver poorly soluble pharmaceutical materials so as to improve the release of such pharmaceutical materials over traditional dosage forms. This could allow for the use of lower dosage levels to deliver equivalent bioavailable levels of drug and thereby lower toxicity levels of both currently marketed drug and new chemical entities. Poorly soluble pharmaceutical-materials can be used in the form of nanoparticles, which are nanometer-sized particles.

In addition to the active ingredient and the granules prepared from low density alkali earth metal salts and/or water soluble carbohydrates, the fast melt tablets can be formulated using conventional carriers or excipients and well established pharmaceutical techniques. Conventional carriers or excipients include, but are not limited to, diluents, binders, adhesives (i.e., cellulose derivatives and acrylic derivatives), lubricants (i.e., magnesium or calcium stearate, vegetable oils, polyethylene glycols, talc, sodium lauryl sulphate, polyoxy ethylene monostearate), disintegrants, colorants, flavorings, preservatives, sweeteners and miscellaneous materials such as buffers and adsorbents. Additional description of the preparation of fast melt tablets can be found, for example, in U.S. Pat. No. 5,939,091, the contents of which are incorporated herein by reference.

Pharmaceutical compositions (including cosmetic preparations) may comprise from about 0.00001 to 100% such as from 0.001 to 10% or from 0.1% to 5% by weight of one or more HAT-modulating compounds described herein. In one embodiment, a HAT-modulating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected earner not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

HAT-modulating compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's (supra) ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

HAT-modulating compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor® from Beiersdorf, Inc. (Norwalk, Conn.). HAT-modulating compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

HAT-modulating compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily-selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

HAT-modulating compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., antioxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C2-C6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol®) and diethylene glycol monoethyl ether oleate (available commercially as Softcutol®); polyethylene castor oil derivatives such as polyoxy castor oil, polyoxy hydro genated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol®); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsiflers and co-emulsifiers described with respect to microemulsion formulations. Emollients include, for example, propylene glycol, glycerol, isopropyl myristate, polypropylene glycol-2 (PP G-2) myristyl ether propionate, and the like. Other active agents may also be included in formulations, e.g., other antiinflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises a modulating compound-containing microemulsion as described above, but may contain alternative pharmaceutically acceptable carriers, vehicles, additives, etc. particularly suited to oral or parenteral drug administration. Alternatively, a modulating compound-containing microemulsion may be administered orally or parenterally substantially as described above, without modification.

Phospholipids complexes, e.g., resveratrol-phospholipid complexes, and their preparation are described in U.S. Patent Application Publication No. 2004/116386. Methods for stabilizing active components using polyol/polymer microcapsules, and their preparation are described in US20040108608. Processes for dissolving lipophilic compounds in aqueous solution with amphiphilic block copolymers are described in WO 04/035013.

Conditions of the eye can be treated or prevented by, e.g., systemic, topical, intraocular injection of a HAT-modulating compound, or by insertion of a sustained release device that releases a sirtuin-modulating compound. A HAT-modulating compound that increases or decreases the level and/or activity of a HAT may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye.

HAT-modulating compounds described herein may be stored in oxygen free environment according to methods in the art. For example, resveratrol or analog thereof can be prepared in an airtight capsule for oral administration, such as Capsugel from Pfizer, Inc.

Cells, e.g., treated ex vivo with a HAT-modulating compound, can be administered according to methods for administering a graft to a subject, which may be accompanied, e.g., by administration of an immunosuppressant drug, e.g., cyclosporin A. For general principles in medicinal formulation, the reader is referred to Cell Therapy. Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of HAT-modulating compounds can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. HAT-modulating compounds that exhibit large therapeutic indexes are preferred. While HAT-modulating compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the EDso with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the ICso (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Kits

Also provided herein are kits, e.g., kits for therapeutic purposes or kits for modulating the lifespan of cells or modulating apoptosis. A kit may comprise one or more HAT-modulating compounds, e.g., in premeasured doses. A kit may optionally comprise devices for contacting cells with the compounds and instructions for use. Devices include syringes, stents and other devices for introducing a HAT-modulating compound into a subject (e.g., the blood vessel of a subject) or applying it to the skin of a subject.

Another type of kit contemplated by the invention are kits for identifying HAT-modulating compounds. Such kits contain (1) a HAT or HAT-containing material and (2) a HAT-modulating compound of the invention, which are in separate vessels. Such kits can be used, for example, to perform a competition-type assay to test other compounds (typically provided by the user) for HAT-modulating activity. In certain embodiments, these kits further comprise means for determining HAT activity.

In yet another embodiment, the invention provides a composition of matter comprising a HAT modulator of this invention and another therapeutic agent [the same ones used in combination therapies and combination compositions] in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen.

The agent and the HAT modulator are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a HAT modulator of this invention; and b) another therapeutic agent such as those described elsewhere in the specification.

The practice, of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2<nd> Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. 5 Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, VoIs. 154 and 155 (Wu et al. eds.), Immunochemical Methods In Cell And Molecular Biology (Mayer and Walker, eds., Academic Press, London, 1987); Handbook Of Experimental Immunology, Volumes HV (D. M. Weir and C. C. Blackwell, eds., 1986); Manipulating the Mouse Embryo, (Cold Spring Harbor Laboratory Press, Cold Spring Harbor, N.Y., 1986).

What is claimed is:

1. A method of selecting or treating a patient for a condition selected from the group consisting of cancer, diabetes, and obesity, the method comprising:
selecting a patient on the basis of that patient being in need of the inhibition of a HAT for the treatment of the condition; and
selecting, and preferably administering to the subject, a compound of described herein.

2. The method of claim 1, further comprising administering to the subject a compound described herein.

3. A method of evaluating a compound described herein the method comprising:
providing a compound described herein;
providing a value related to the ability of the compound to inhibit a HAT; and
optionally, comparing the value with a reference value,
thereby evaluating the compound.

4. The method of claim 3, further comprising assaying the compound to determining a value related to the ability of the compound to inhibit a HAT.
